(19) United States
(12) Patent Application Publication
(10) Pub. No.: US 2025/0034559 A1
(43) Pub. Date: Jan. 30, 2025

(54) COMPOSITIONS AND METHODS FOR THE TREATMENT OF TAU-RELATED DISORDERS

(71) Applicant: Voyager Therapeutics, Inc., Lexington, MA (US)

(72) Inventors: Mathieu Emmanuel NONNENMACHER, Boston, MA (US); Matthew Alan CHILD, Ashland, MA (US); Jinzhao HOU, Lexington, MA (US); Jiangyu LI, Watertown, MA (US); Shaoyong LI, Hopkinton, MA (US); Tyler Christopher MOYER, Boston, MA (US); Wei WANG, Arlington, MA (US); Brett FOFFMAN, Braintree, MA (US); Elisabeth KNOLL, Melrose, MA (US); Yanqun SHU, Winchester, MA (US); Nilesh Navalkishor PANDE, North Grafton, MA (US); Jeffrey Scott THOMPSON, Stoneham, MA (US)

(21) Appl. No.: 18/711,059

(22) PCT Filed: Nov. 17, 2022
(86) PCT No.: PCT/US2022/080040

§ 371 Date: May 16, 2024

Related U.S. Application Data
(60) Provisional application No. 63/280,485, filed on Nov. 17, 2021.

(51) Int. Cl. C12N 15/11; A61K 31/7088; A61K 45/06; A61P 25/28; C07K 16/18; C12N 5/071

(52) U.S. Cl. CPC C12N 15/11; A61K 31/7088; A61K 45/06; A61P 25/28; C07K 16/18; C12N 5/0602; C12N 2510/02; C12N 2750/14142; C12N 2750/14171

(57) ABSTRACT

The disclosure provides compositions and methods for the preparation, manufacture and use of an adeno-associated virus (AAV) particle for the vectorized delivery of an antibody molecule that binds to tau.

### RELATED APPLICATIONS

This application claims priority to U.S. Provisional Application No. 63/280,485 filed on Nov. 17, 2021; the entire contents of which are hereby incorporated by reference in their entirety.

### FIELD OF THE DISCLOSURE

Described herein are compositions and methods relating to AAV particles for delivery of an anti-tau antibody molecule for use in the treatment of tau-related disorders (e.g., tauopathies).

### BACKGROUND

Tauopathies are a group of neurodegenerative diseases characterized by the dysfunction and/or aggregation of the microtubule associated protein tau. Tau is normally a very soluble protein known to associate with microtubules based on the extent of its phosphorylation. Tau is considered a critical component of intracellular trafficking processes, particularly in neuronal cells, given their unique and extended structure. Hyperphosphorylation of tau depresses its binding to microtubules and microtubule assembly activity. Further, hyperphosphorylation of tau renders it prone to misfolding and aggregation. In tauopathies, the tau becomes hyperphosphorylated, misfolds and aggregates as neurofibrillary tangles (NFT) of paired helical filaments (PHF), twisted ribbons or straight filaments. These NFT are largely considered indicative of impending neuronal cell death and thought to contribute to widespread neuronal cell loss, leading to a variety of behavioral and cognitive deficits.

Several approaches have been proposed for therapeutically interfering with progression of tau pathology and preventing the subsequent molecular and cellular consequences. Given that NFT are composed of hyperphosphorylated, misfolded and aggregated forms of tau, interference at each of these stages provides targets that can be pursued. Introducing agents that limit phosphorylation, block misfolding or prevent aggregation are promising strategies. It has also been suggested that introduction of anti-tau antibodies can prevent the trans-neuronal spread of tau pathology.

There remains a need for anti-tau antibodies for use in tauopathy treatment, diagnostics, and other applications. The present disclosure addresses this need with related compounds and methods described herein.

### SUMMARY

The present disclosure addresses the need in the art for improved methods for delivery of antibodies and antibody-based therapeutics targeting tau by providing novel AAV particles having viral genomes engineered to encode anti-tau antibody molecules and antibody-based compositions and methods of using the same for the treatment, prevention, diagnosis, and research of diseases, disorders and/or conditions associated with tau pathology.

In one aspect, provided herein is an isolated, e.g., recombinant, AAV particle comprising an AAV capsid variant and a nucleic acid comprising a transgene encoding an antibody molecule which binds to tau (e.g., human tau), wherein the AAV capsid variant (i) is enriched 5- to 400-fold in the brain (e.g., a brain region of an NHP) compared to SEQ ID NO: 138; (ii) transduces a brain region (e.g., dentate nucleus, cerebellar cortex, cerebral cortex, brain stem, hippocampus, thalamus and putamen), wherein, e.g., the level of transduction is at least 5- to 10,000-fold greater as compared to a reference sequence of SEQ ID NO: 138; (iii) delivers an increased level of a payload to a brain region (e.g., frontal cortex, sensory cortex, motor cortex, putamen, thalamus, cerebellar cortex, dentate nucleus, caudate, and/or hippocampus), wherein, e.g., the level of the payload is increased by at least 500- to 10,00-fold; (iv) delivers an increased level of a payload to a spinal cord region (e.g., cervical, thoracic, and/or lumbar region), wherein, e.g., the level of the payload is increased by at least 10- to 900-fold, as compared to a reference sequence of SEQ ID NO: 138; and/or (v) delivers an increased level of viral genomes to a brain region (frontal cortex, sensory cortex, motor cortex, putamen, thalamus, cerebellar cortex, dentate nucleus, caudate, and/or hippocampus), wherein, e.g., the level of viral genomes is increased by at least 5- to 50-fold as compared to a reference sequence of SEQ ID NO: 138. In some embodiments, the level of payload is measured using qRT-PCR or RT-ddPCR.

In another aspect, provided herein is an isolated, e.g., recombinant, AAV particle comprising an AAV capsid variant and a nucleic acid comprising a transgene encoding an antibody molecule which binds to tau, wherein the AAV capsid variant comprises (a) the amino acid sequence of any of SEQ ID NO: 3648-3659 or a sequence provided in Table 20, (b) an amino acid sequence comprising at least 5, 6, 7, 8, or 9 consecutive amino acids from the amino acid sequence of any of SEQ ID NO: 3648-3659 or a sequence provided in Table 20, (c) an amino acid sequence comprising at least one, two, or three but no more than four modifications, e.g., substitutions, (e.g., conservative substitutions), insertions, or deletions, relative to the amino acid sequence of any of SEQ ID NOs: 3648-3659 or a sequence provided in Table 20, or (d) an amino acid sequence comprising at least one, two, or three but no more than four different amino acids relative to the amino acid sequence of any of SEQ ID NOs: 3648-3659 or a sequence provided in Table 20. In some embodiments, the capsid variant comprises the amino acid sequence of SEQ ID NO: 138, or an amino acid sequence with at least 95% sequence identity thereto.

In another aspect, provided herein is an isolated, e.g., recombinant, AAV particle comprising an AAV capsid variant and a nucleic acid comprising a transgene encoding an antibody molecule which binds to tau (e.g., human tau), wherein the AAV capsid variant comprises: (i) the amino acid sequence of PLNGAVHLY (SEQ ID NO: 3648); (ii) an amino acid sequence comprising at least 5, 6, 7, 8, or 9 consecutive amino acids from the amino acid sequence of PLNGAVHLY (SEQ ID NO: 3648); (iii) an amino acid sequence comprising at least one, two, or three but no more than four modifications, e.g., substitutions (e.g., conservative substitutions), insertions, or deletions, relative to the amino acid sequence of PLNGAVHLY (SEQ ID NO: 3648); or (iv) an amino acid sequence comprising at least one, two, or three but no more than four different amino acids relative to the amino acid sequence of PLNGAVHLY (SEQ ID NO: 3648). In some embodiments, the capsid variant comprises the amino acid sequence of SEQ ID NO: 138, or an amino acid sequence with at least 95% sequence identity thereto.

In yet another aspect, provided herein is an isolated, e.g., recombinant, AAV particle comprising an AAV capsid variant and a nucleic acid comprising a transgene encoding an antibody molecule which binds to tau (e.g., human tau), wherein the AAV capsid variant comprises an amino sequence comprising the following formula: [N1]−[N2], wherein: (i) [N1] comprises X1, X2, X3, X4, and X5, wherein: (a) position X1 is: P, Q, A, H, K, L, R, S, or T; (b) position X2 is: L, I, V, H, or R; (c) position X3 is: N, D, I, K, or Y; (d) position X4 is: G, A, C, R, or S; and (e) position X5 is: A, S, T, G, C, D, N, Q, V, or Y; and (ii) [N2] comprises the amino acid sequence of VHLY, VHIY, VHVY, or VHHY; and/or an amino acid modification, e.g., a conservative substitution, of any of the aforesaid amino acids in (i) and/or (ii).

In yet another aspect, provided herein is an isolated, e.g., recombinant, AAV particle comprising an AAV capsid variant and a nucleic acid comprising a transgene encoding an antibody molecule which binds to tau (e.g., human tau), wherein the AAV capsid variant comprises one, two, three, four, or all of: (i) an [N1], wherein [N1] is: SLNGA, QLNGA, ALNGA, PLNGS, PVNGA, PLNGA, PLNGG, PLNGT, PLDGA, QLNGS, PLNGN, SLDGA, HLNGA, ALNGT, PINGA, ALDGA, PLNCA, PLNGQ, PLDSA, RLDGA, QLNGN, PLNGY, PLDSS, PLNGC, PLYGA, TLNGA, PVDGA, PLKGA, PLNGD, KLDGA, PHNGA, PLNGV, PLNAA, QLNGY, PLDGS, LLNGA, PLNRA, PLIGA, PRNGA, or ALNGS; (ii) an [N2] wherein [N2] is: VHLY, VHVY, VPLY, VNLY, VHRY, VHIY, VHHY, FHLY, LHLY, DHLY, VQLY, IHLY, VDLY, AHLY, VLLY, GHLY, VRLY, or VYLY; (iii) an [N3] wherein [N3] is: AQAQ, SQAQ, AQPQ, AQSQ, AKAQ, AHAQ, AQAP, DQAQ, APAQ, AQAK, AQAH, AQEQ, ALAQ, ARAQ, or TQAQ; (iv) an [N4] wherein [N4] is: TGW, TGL, TGS, TGG, TAW, TGR, TAS, LSS, TSS, SSL, SSS, TLS, TVS, VSS, TSP, VSP, TMS, LSP, VAS, TAL, TTS, TLP, VLP, RGW, LSG, LAS, SSP, LLP, STS, TSA, TTP, SAL, LGS, VTP, VSA, IGW, TGF, LTP, TLA, LSA, TVG, TAP, TMP, TSL, VQS, SSM, SLP, VSQ, RSS, TST, VMS, TTA, TQP, LST, LAP, TVA, RLS, TGY, TSG, TAG, VMP, TSQ, TMA, VGS, TSW, TGV, TGT, TLG, LMP, VQP, TGM, SMS, SQL, IGS, RSV, TAA, STP, LSQ, TAQ, TGP, ASP, VSG, SAP, TLQ, LQP, TAT, TGQ, ATS, IGG, VAA, TSM, TVW, TAM, TGA, VAT, QSP, TQA, VQA, RSP, LAT, VAQ, LAA, RST, RTL, LGT, LMS, LGP, RTS, SQP, VLG, SVS, TMQ, SAV, LAG, SGP, TNS, RLT, TTQ, SAA, TSV, RLG, RAS, STQ, CSP, SAG, ALP, VTS, ISP, SVG, LTS, TTT, RSG, TQL, LNP, TVQ, IAS, LAQ, LSR, LSN, TTG, TSN, SMA, TKS, SVA, TQQ, VQQ, RLP, SAM, TAV, TQW, SSR, TQT, VNS, RSA, LMG, RQS, LVG, VTA, RTT, SMG, VMA, TKP, SAQ, NSP, ATP, VAG, RGS, VKP, RMS, NLP, NAL, RTP, RQL, VQG, VTG, VST, NAS, RVE, ATG, AMS, RNS, VMQ, SMQ, LQQ, TMG, LGQ, TSH, AAP, RSQ, TYS, ITP, VAK, TQM, TKA, SQQ, ISG, VSR, RTA, RML, SQM, VAN, CTP, ISS, AGP, TAK, RTG, LHP, TMT, AQP, QAP, RQP, LKS, NTT, TSK, RYS, KSS, NTP, VGG, IAA, LMA, MAP, VHP, VLS, LAN, ATQ, TNA, TAN, VSN, AAA, AVG, LTA, SAN, RAG, RQG, TLR, LSH, SAF, RAA, IQP, ILG, VNG, SVQ, LSK, TNG, RTQ, TMN, RGG, TTR, VRP, VKA, LAR, NQP, TMK, TYA, TQK, TTK, IAG, TQN, LAH, NTQ, RQQ, RAQ, TKQ, TQH, TNQ, LMQ, VNA, VQT, TQR, VGK, VKQ, IQS, LQR, TMM, VGN, RIG, SAK, RIA, VQN, NVQ, RIP, NAQ, NMQ, TPS, LTN, VTK, PGW, LPP, SPP, TPA, TGC, VPP, TPT, TPW, TPP, RPP, TPQ, TPR, TPG, VPA, VPQ, RPG, KGW, TRW, TAR, IPP, RSL, LVP, KGS, VAP, KGG, KAW, PGS, TRL, or AGW; and/or (v) an [N5] wherein [N5] is: VQN, VKN, VQT, VQK, DQN, VQH, GQN, VQI, VHN, FQN, LQN, VLN, VRN, VQS, VQY, AQN, VEN, VQD, VPN, IQN, VKK, DKN, VKT, VQP, EQN, GQT, FQK, GHN, or VPH; and/or wherein the AAV capsid variant comprises an amino acid modification, e.g., a conservative substitution, of any of the aforesaid amino acids in (i)-(v).

In yet another aspect, provided herein is an isolated, e.g., recombinant, AAV particle comprising an AAV capsid variant and a nucleic acid comprising a transgene encoding an antibody molecule which binds to tau (e.g., human tau), wherein the AAV capsid variant comprises one, two, three, four, or all of: (i) an [N1], wherein [N1] is: SLNGA, QLNGA, ALNGA, PLNGS, PVNGA, PLNGA, PLNGG, PLNGT, PLDGA, QLNGS, PLNGN, SLDGA, HLNGA, ALNGT, PINGA, ALDGA, PLNCA, PLNGQ, PLDSA, RLDGA, QLNGN, PLNGY, or PLDSS; (ii) an [N2] wherein [N2] is: VHLY or VHVY; (iii) an [N3] wherein [N3] is: AQAQ, SQAQ, AQPQ, or AQSQ; (iv) an [N4] wherein [N4] is: TGW, TGL, TGS, TGG, TAW, TGR, TAS, LSS, TSS, SSL, SSS, TLS, TVS, VSS, TSP, VSP, TMS, LSP, VAS, TAL, TTS, TLP, VLP, RGW, LSG, LAS, SSP, LLP, STS, TSA, TTP, SAL, LGS, VTP, VSA, IGW, TGF, LTP, TLA, LSA, TVG, TAP, TMP, TSL, VQS, SSM, SLP, VSQ, RSS, TST, VMS, TTA, TQP, LST, LAP, TVA, RLS, TGY, TSG, TAG, VMP, TSQ, TMA, VGS, TSW, TGV, TGT, TLG, LMP, VQP, TGM, SMS, SQL, IGS, RSV, TAA, STP, LSQ, TAQ, TGP, ASP, VSG, SAP, TLQ, LQP, TAT, TGQ, ATS, IGG, VAA, TSM, TVW, TAM, TGA, VAT, QSP, TQA, VQA, RSP, LAT, VAQ, LAA, RST, RTL, LGT, LMS, LGP, RTS, SQP, VLG, SVS, TMQ, SAV, LAG, SGP, TNS, RLT, TTQ, SAA, TSV, RLG, RAS, STQ, CSP, SAG, ALP, VTS, ISP, SVG, LTS, TTT, RSG, TQL, LNP, TVQ, IAS, LAQ, LSR, LSN, TTG, TSN, SMA, TKS, SVA, TQQ, VQQ, RLP, SAM, TAV, TQW, SSR, TQT, VNS, RSA, LMG, RQS, LVG, VTA, RTT, SMG, VMA, TKP, SAQ, NSP, ATP, VAG, RGS, VKP, RMS, NLP, NAL, RTP, RQL, VQG, VTG, VST, NAS, RVE, ATG, AMS, RNS, VMQ, SMQ, LQQ, TMG, LGQ, TSH, AAP, RSQ, TYS, ITP, VAK, TQM, TKA, SQQ, ISG, VSR, RTA, RML, SQM, VAN, CTP, ISS, AGP, TAK, RTG, LHP, TMT, AQP, QAP, RQP, LKS, NTT, TSK, RYS, KSS, NTP, VGG, IAA, LMA, MAP, VHP, VLS, LAN, ATQ, TNA, TAN, VSN, AAA, AVG, LTA, SAN, RAG, RQG, TLR, LSH, SAF, RAA, IQP, ILG, VNG, SVQ, LSK, TNG, RTQ, TMN, RGG, TTR, VRP, VKA, LAR, NQP, TMK, TYA, TQK, TTK, IAG, TQN, LAH, NTQ, RQQ, RAQ, TKQ, TQH, TNQ, LMQ, VNA, VQT, TQR, VGK, VKQ, IQS, LQR, TMM, VGN, RIG, SAK, RIA, VQN, NVQ, RIP, NAQ, NMQ, TPS, LTN, VTK, PGW, LPP, SPP, TPA, or TGC; and/or (v) an [N5] wherein [N5] is independently chosen from VQN, VKN, VQT, VQK, DQN, VQH, GQN, VQI, VHN, FQN, LQN, VLN, VRN, VQS, VQY, AQN, VEN, VQD; and/or wherein the AAV capsid variant comprises an amino acid modification, e.g., a conservative substitution, of any of the aforesaid amino acids in (i)-(v).

In yet another aspect, provided herein is an isolated, e.g., recombinant, AAV particle comprising an AAV capsid variant and a nucleic acid comprising a transgene encoding an antibody molecule which binds to tau (e.g., human tau), wherein the AAV capsid variant comprises [A][B], wherein [A] comprises the amino acid sequence of PLNGA, and [B] comprises X1, X2, X3, X4, wherein: (i) X1 is: V, I, L, A, F, D, or G; (ii) X2 is: H, N, Q, P, D, L, R, or Y; (iii) X3 is: L, H, I, R, or V; and (iv) X4 is Y; and/or wherein the AAV capsid variant comprises an amino acid modification, e.g., a conservative substitution, of any of the aforesaid amino acids in (i)-(iv). In some embodiments, the AAV capsid variant further comprises one, two, or all of an amino acid other than T at position 593 (e.g., a V, S, L, R, I, A, N, C, Q, M, P, or K), an amino acid other than G at position 594 (e.g., T, M, A, K, S, Q, V, I, R, N, P, L, H, or Y), and/or an amino acid other than W at position 595 (e.g., K, Q, S, P, C, A, G, N, T, R, V, M, H, L, E, F, or Y), relative to a reference sequence numbered according to the amino acid sequence of SEQ ID NO: 138.

In yet another embodiment, provided herein is an isolated, e.g., recombinant, AAV particle comprising an AAV capsid variant and a nucleic acid comprising a transgene encoding an antibody molecule which binds to tau (e.g., human tau), wherein the AAV capsid variant comprises PLNGAVHLY (SEQ ID NO: 3648) and optionally wherein the AAV capsid variant further comprises one, two, or all of an amino acid other than T at position 593 (e.g., A, L, R, V, C, I, K, M, N, P, Q, S), an amino acid other than G at position 594 (e.g., M, S, A, Q, V, T, L, P, H, K, N, I, Y, or R), and/or an amino acid other than W at position 595 (e.g., S, P, T, A, G, L, Q, H, N, R, K, V, E, F, M, C, or Y), relative to a reference sequence numbered according to the amino acid sequence of SEQ ID NO: 138.

In yet another embodiment, provided herein is an isolated, e.g., recombinant, AAV particle comprising an AAV capsid variant and a nucleic acid comprising a transgene encoding an antibody molecule which binds to tau (e.g., human tau), wherein the AAV capsid variant comprises an amino sequence comprising the amino acid sequence of PLNGAVHLY(SEQ ID NO: 3648); and which further comprises one, two, three, or all of: (i) the amino acid at position 593, relative to a reference sequence numbered according to the amino acid sequence of SEQ ID NO: 138, is: T, A, L, R, V, C, I, K, M, N, P, Q, or S; (ii) the amino acid at position 594, relative to a reference sequence numbered according to the amino acid sequence of SEQ ID NO: 138, is: G, M, S, A, Q, V, T, L, P, H, K, N, I, Y, or R; and/or (iii) the amino acid at position 595, relative to a reference sequence numbered according to the amino acid sequence of SEQ ID NO: 138, is: W, S, P, T, A, G, L, Q, H, N, R, K, V, E, F, M, C, or Y. In some embodiments, the amino acids at positions 593-595, relative to a reference sequence numbered according to the amino acid sequence of SEQ ID NO: 138, does not comprise the amino acid sequence of TGW.

In yet another aspect, provided herein is an isolated, e.g., recombinant, AAV particle comprising an AAV capsid variant and a nucleic acid comprising a transgene encoding an antibody molecule which binds to tau (e.g., human tau), wherein the AAV capsid variant comprises X1-X2-X3-X4-X5-X6-X7-X8-X9-X10-X11-X12-X13-X14-X15-X16-X17-X18-X19, wherein: (i) X1 is: P, A, D, E, F, G, H, K, L, N, Q, R, S, T, or V; (ii) X2 is: L, D, E, F, H, I, M, N, P, Q, R, S, or V; (iii) X3 is: N, A, D, E, G, H, I, K, Q, S, T, V, or Y; (iv) X4 is: G, A, C, D, E, P, Q, R, S, T, V, or W; (v) X5 is: A, C, D, E, F, G, H, I, K, N, P, Q, R, S, T, V, W, or Y; (vi) X6 is: V, A, C, D, E, F, G, H, I, K, L, M, N, Q, R, S, T, or Y; (vii) X7 is: H, A, D, E, G, I, K, L, M, N, P, Q, R, S, T, V, or Y; (viii) X8 is: L, A, D, E, F, G, H, I, K, M, N, P, Q, R, S, T, V, or Y; (ix) X9 is: Y, A, C, D, E, F, G, H, I, K, L, M, N, Q, R, S, T, V, or W; (x) X10 is: A, C, D, E, F, G, H, I, K, L, N, P, Q, R, S, T, V, or; Y; (xi) X11 is: Q, A, D, E, H, K, L, P, R, or T; (xii) X12 is: A, D, E, G, H, L, N, P, Q, R, S, T, or V; (xiii) X13 is: Q, E, H, K, L, P, R, or T; (xiv) X14 is: T, A, C, D, E, F, G, H, I, K, L, M, N, P, Q, R, S, V, W, or Y; (xv) X15 is: G, A, C, D, E, F, H, I, K, L, M, N, P, Q, R, S, T, V, W, or Y; (xvi) X16 is: W, A, C, D, E, F, G, H, I, K, L, M, N, P, Q, R, S, T, V, or Y; (xvii) X17 is: V, A, D, E, F, G, H, I, or L; (xviii) X18 is: Q, E, H, K, L, P, or R; and/or (xix) X19 is: N, D, H, I, K, P, S, T, or Y.

In yet another aspect, provided herein is an isolated, e.g., recombinant, AAV particle comprising an AAV capsid variant and a nucleic acid comprising a transgene encoding an antibody molecule which binds to tau (e.g., human tau), wherein the AAV capsid variant comprises: (a) the amino acid sequence of any one of SEQ ID NOs: 139-1138; (b) an amino acid sequence comprising at least 3, 4, 5, 6, 7, 8, 9, 10, 11, 12, 13, 14, 15, 16, 17, or 18 consecutive amino acids from any one of SEQ ID NOs: 139-1138; (c) an amino acid sequence comprising at least one, two, or three but no more than four different amino acids, relative to the amino acid sequence of any one of SEQ ID NOs: 139-1138; or (d) an amino sequence comprising at least one, two, or three but no more than four modifications, e.g., substitutions (e.g., conservative substitutions), insertions, or deletions, relative to the amino acid sequence of any one of SEQ ID NOs: 139-1138. In some embodiments, the AAV capsid variant does not comprise the amino acid sequence of TGW at positions 593-595, relative to a reference sequence numbered according to the amino acid sequence of SEQ ID NO: 138.

In some embodiments, the AAV capsid variant does not comprise: (i) the amino acid sequence of TLAVPFK (SEQ ID NO: 1262) present immediately subsequent to position 588, numbered according to SEQ ID NO: 138; (ii) an amino acid sequence present immediately subsequent to position 586 to 599, e.g., 586 to 594, 587 to 595, 588 to 596, 589 to 597, 590 to 598 numbered relative to SEQ ID NO: 138, having at least 5 consecutive amino acids corresponding to positions 586 to 594 numbered relative to SEQ ID NO: 138, of any the amino acid sequences provided in Table 1 of WO2020223276, the contents of which are hereby incorporated by reference in their entirety; or (iii) an amino acid sequence present immediately subsequent to position 586 to 599, e.g., 586 to 594, 587 to 595, 588 to 596, 589 to 597, 590 to 598 numbered relative to SEQ ID NO: 138, having at least 5 consecutive amino acids corresponding to positions 586 to 594 numbered relative to SEQ ID NO: 138, of any SEQ ID NOs: 1, 12, 13, or 138.

In some embodiments, the anti-tau antibody molecule binds to the N-terminal region, mid-domain region, C-terminal region, microtubule binding domain, or the proline-rich domain of a tau protein (e.g., a human tau protein comprising the amino acid sequence of SEQ ID NO: 9200). In some embodiments, the anti-tau antibody molecule binds to (a) all or a portion of (e.g., one or more residues within) amino acid residues (1) 9-18, (2) 15-25, (3) 25-30, and/or (4) 15-30, numbered according to SEQ ID NO: 9200; (b) all or a portion of (e.g., one or more residues within) amino acid residues (1) 125-131, (2) 204-222, (3) 234-246, (4) 234-259, and/or (5) 235-246, numbered according to SEQ ID NO: 9200; (c) an epitope which includes one or more of pT181, pS199, pS202, pT205, pT212, pS214, pT217, pT231, pS234, pS235, pS258, pS259, pS396, pS404, and/or pT217, numbered according to SEQ ID NO: 9200; (d) all or a portion of (one or more residues within) amino acid residues (1) 387-408 and/or (2) 409-436, numbered according to SEQ ID NO: 9200; (e) all or a portion of (e.g., one or more residues within) amino acid residues (1) 32-49, (2) 55-76, (3) 57-72, (4) 159-194, (5) 175-191, (6) 185-200, (7) 219-247, (8) 223-238, (9) 381-426, (10) 383-400, (11) 409-436, and/or (12) 413-430, numbered according to SEQ ID NO: 9200; (f) a conformational epitope which includes all or a portion of (e.g., one or more residues within) amino acid residues (1) 55-76, (2) 159-194, (3) 219-247, and/or (4) 381-426, numbered according to SEQ ID NO: 9200.

In some embodiments, the anti-tau antibody molecule comprises: (i) a heavy chain variable region comprising one, two, or three HCDR sequences of any one of the anti-tau antibody molecules described herein (e.g., an antibody molecule listed in Tables 7-16); and/or (ii) a light chain variable region comprising one, two, or three LCDR sequences of any one of the anti-tau antibody molecules described herein (e.g., an antibody molecule listed in Tables 7-16).

In some embodiments, the anti-tau antibody molecule comprises: (i) a heavy chain variable region (VH) comprising the amino acid sequence of the VH of any one of the anti-tau antibody molecules described herein (e.g., the VH sequence of an antibody molecule listed in Tables 7-16), or an amino acid sequence having at least 80% (e.g., 85, 90, 95, 96, 97, 98, or 99%) sequence identity to the VH sequence of any one of the anti-tau antibody molecules described herein (e.g., the VH sequence of an antibody molecule listed in Tables 7-16), or an amino acid sequence having at least one, two or three modifications, but not more than 30, 20 or 10 modifications, to the amino acid sequence of the VH of any of the anti-tau antibody molecules described herein (e.g., the VH sequence of an antibody molecule listed in Tables 7-16); and/or (ii) a light chain variable region (VL) comprising the amino acid sequence of the VL of any one of the anti-tau antibody molecules described herein (e.g., the VL sequence of an antibody molecule listed in Tables 7-16), or an amino acid sequence having at least 80% (e.g., 85, 90, 95, 96, 97, 98, or 99%) sequence identity to the VL sequence of any one of the anti-tau antibody molecules described herein (e.g., the VL sequence of an antibody molecule listed in Tables 7-16), or an amino acid sequence having at least one, two or three modifications, but not more than 30, 20 or 10 modifications, to the amino acid sequence of the VL of any of the anti-tau antibody molecules described herein (e.g., the VL sequence of an antibody molecule listed in Tables 7-16).

In some embodiments, the anti-tau antibody molecule comprises (a) the HCDR1, HCDR2, and/or HCDR3 sequences, and/or LCDR1, LCDR2, and/or LCDR3 sequences, (b) the VH and/or VL sequences, or (c) the heavy chain and/or light chain sequences of IPN002, AT8, PT3, UCB, PT76, PHF1, C10.2, V0004, V0009, V0022, V0023, V0024, and V0052 as described, e.g., in WO2020223276 and WO2021/211753. In some embodiments, the anti-tau antibody molecule has at most one, two, or three substitutions (e.g., conservative amino acid substitutions) in each CDR relative to the HCDR1-3 and LCDR1-3 sequences of IPN002, AT8, PT3, UCB, PT76, PHF1, C10.2, V0004, V0009, V0022, V0023, V0024, or V0052. In some embodiments, the anti-tau antibody molecule comprises a VH sequence having at least 80% (e.g., 85, 90, 95, 96, 97, 98, or 99%) sequence identity to the VH sequence of any one of IPN002, AT8, PT3, UCB, PT76, PHF1, C10.2, V0004, V0009, V0022, V0023, V0024, or V0052, and/or a VL sequence having at least 80% (e.g., 85, 90, 95, 96, 97, 98, or 99%) sequence identity to the VL sequence of any one of IPN002, AT8, PT3, UCB, PT76, PHF1, C10.2, V0004, V0009, V0022, V0023, V0024, or V0052. In some embodiments, the anti-tau antibody molecule comprises a VH sequence having at least one, two, or three modifications, but not more than 30, 20, or 10 modifications, relative to the VH sequence of any one of IPN002, AT8, PT3, UCB, PT76, PHF1, C10.2, V0004, V0009, V0022, V0023, V0024, and V0052, and/or a VL sequence having at least one, two, or three modifications, but not more than 30, 20, or 10 modifications, relative to the VL sequence of any one of IPN002, AT8, PT3, UCB, PT76, PHF1, C10.2, V0004, V0009, V0022, V0023, V0024, and V0052.

In some embodiments, the anti-tau antibodies have a heavy chain constant region and/or light chain constant region listed in Table 17, or a sequence having at least 80% (e.g., 85, 90, 95, 96, 97, 98, or 99%) sequence identity to the heavy chain and/or light chain constant region sequences in Table 17, or an amino acid sequence having at least one, two or three modifications, but not more than 30, 20 or 10 modifications, to the amino acid sequence of the heavy chain and/or light chain constant region sequences in Table 17.

In some embodiments, the anti-tau antibody molecule competes for binding to tau with an antibody described herein (e.g., an antibody molecule listed in Tables 7-16). In some embodiments, the anti-tau antibody molecule binds to the same epitope, substantially the same epitope as, an epitope that overlaps with, or an epitope that substantially overlaps with an antibody described herein (e.g., an antibody molecule listed in Tables 7-16).

In some embodiments, the anti-tau antibody molecule comprises one or more of (a) an Fc region or functional variant thereof, e.g., an Fc region with altered effector function (e.g., reduced affinity for Fc receptor, reduced ADCC, reduced CDC), (b) a signal sequence, (c) a linker (e.g., a linker listed in Table 19).

In some embodiments, the anti-tau antibody molecule is a multispecific antibody molecule comprising at least two antigen-binding domains (e.g., a bispecific antibody molecule).

In some embodiments, the AAV particle comprises a viral genome comprising one or more of the following: a promoter operably linked to the nucleic acid sequence encoding the antibody molecule, a poly A sequence, an inverted terminal repeat (ITR) sequence (positioned 5′ and/or 3′ relative to the encoded antibody molecule), an enhancer, a Kozak sequence (e.g., GCCGCCACCATG (SEQ ID NO: 9021) or GAGGAGCCACC (SEQ ID NO: 9022)), an intron region, and/or an exon region described herein. In some embodiments, the viral genome comprises a nucleotide sequence encoding a miR binding site described herein.

In some embodiments, the viral genome is single stranded or self-complementary. In some embodiments, the viral genome further comprises a nucleotide sequence encoding a Rep protein described herein.

Also provided herein are cells (e.g., a host cell) comprising an AAV particle described herein. In some embodiments, the cell is a mammalian cell (e.g., a cell of a brain region, such as a neuron, astrocyte, or glial cell) or an insect cell.

In yet another aspect, provided herein is a method of making an AAV particle described herein, comprising (a) providing a host cell comprising a viral genome; and (b) incubating the host cell under conditions suitable to enclose the viral genome in an AAV capsid variant, e.g., an AAV capsid variant described herein, thereby making the AAV particle.

In yet another aspect, provided herein is a pharmaceutical composition comprising an AAV particle described herein, and a carrier (e.g., a pharmaceutically acceptable excipient).

In yet another aspect, provided herein is a method of delivering a payload to a cell or tissue (e.g., CNS cell or CNS tissue) comprising administering an effective amount of a pharmaceutical composition described herein or an AAV particle described herein. In some embodiments, the cell or tissue is within a subject.

In yet another aspect, provided herein is a method of treating a subject having or diagnosed with having a neurological disorder (e.g., a neurodegenerative disorder), tau-related disease, or tauopathy, comprising administering to the subject an effective amount of the pharmaceutical composition described herein, or an AAV particle described herein. In some embodiments, the AAV particles, compositions, and methods may, for example, be used for treating a tau-related disorder (e.g., disease associated with expression of tau, neurological (e.g., neurodegenerative) disorders, and/or tauopathies), such as Alzheimer's disease (AD), frontotemporal dementia and parkinsonism linked to chromosome 17 (FTDP-17), frontotemporal lobar degeneration (FTLD), frontotemporal dementia (FTD), chronic traumatic encephalopathy (CTE), progressive Supranuclear Palsy (PSP), Down's syndrome, Pick's disease, corticobasal degeneration (CBD), corticobasal syndrome, amyotrophic lateral sclerosis (ALS), prion diseases, Creutzfeldt-Jakob disease (CJD), multiple system atrophy, tangle-only dementia, and progressive subcortical gliosis.

In some embodiments, the AAV particle is administered to the subject intravenously, via intra-cisterna magna injection (ICM), intracerebrally, intrathecally, intracerebroventricularly, via intraparenchymal administration, or intramuscularly, or via focused ultrasound (FUS), e.g., coupled with the intravenous administration of microbubbles (FUS-MB), or MRI-guided FUS coupled with intravenous administration.

In some embodiments, administration of the AAV particle or pharmaceutical composition described herein results in a decreased presence, level, and/or activity of a tau mRNA, tau protein, or combination thereof. In other embodiments, administration of the AAV particle or pharmaceutical composition described herein results in a increased presence, level, and/or activity of a tau mRNA, tau protein, or combination thereof.

Those skilled in the art will recognize or be able to ascertain using no more than routine experimentation, many equivalents to the specific embodiments of the invention described herein. Such equivalents are intended to be encompassed by the following enumerated embodiments.

### Enumerated Embodiments

1. An isolated, e.g., recombinant, AAV particle comprising an AAV capsid variant and a nucleic acid comprising a transgene encoding an antibody molecule which binds to tau (e.g., human tau), wherein the AAV capsid variant:
   (i) is enriched at least about 5, 6, 7, 8, 9, 10, 20, 30, 40, 50, 100, 200, 300, or 400-fold, in the brain, e.g., the brain of a non-human primate (NHP) compared to a reference sequence of SEQ ID NO: 138 (e.g., as provided in Table 1), e.g., when measured by an assay as described in Example 1;
   (ii) transduces a brain region, e.g., a brain region of an NHP, e.g., selected from dentate nucleus, cerebellar cortex, cerebral cortex, brain stem, hippocampus, thalamus and putamen, wherein the level of transduction is at least 5, 10, 50, 100, 200, 500, 1,000, 2,000, 5,000, or 10,000-fold greater as compared to a reference sequence of SEQ ID NO: 138, e.g., when measured by an assay, e.g., an immunohistochemistry assay, a qRT-PCR, or a RT-ddPCR assay, e.g., as described in Example 2;
   (iii) delivers an increased level of a payload to a brain region, e.g., a brain region of an NHP, optionally wherein the level of the payload is increased by at least 500, 1,000, 2,000, 5,000, or 10,000-fold, as compared to a reference sequence of SEQ ID NO: 138, e.g., when measured by an assay, e.g., a qRT-PCR or a RT-ddPCR assay (e.g., as described in Example 2), optionally wherein the brain region comprises a frontal cortex, sensory cortex, motor cortex, putamen, thalamus, cerebellar cortex, dentate nucleus, caudate, and/or hippocampus;
   (iv) delivers an increased level of a payload to a spinal cord region, e.g., a spinal cord region of an NHP, optionally wherein the level of the payload is increased by at least 10, 20, 50, 100, 200, 300, 400, 500, 600, 700, 800 or 900-fold, as compared to a reference sequence of SEQ ID NO: 138, e.g., when measured by an assay, e.g., a qRT-PCR assay (e.g., as described in Example 2), optionally wherein the spinal cord region comprises a cervical, thoracic, and/or lumbar region; and/or
   (v) delivers an increased level of viral genomes to a brain region, e.g., a brain region of an NHP, optionally wherein the level of viral genomes is increased by at least 5, 10, 20, 30, 40 or 50-fold, as compared to a reference sequence of SEQ ID NO: 138, e.g., when measured by an assay, e.g., a qRT-PCR or a RT-ddPCR assay (e.g., as described in Example 2), optionally wherein the brain region comprises a frontal cortex, sensory cortex, motor cortex, putamen, thalamus, cerebellar cortex, dentate nucleus, caudate, and/or hippocampus.
2. An isolated, e.g., recombinant, AAV particle comprising an AAV capsid variant and a nucleic acid comprising a transgene encoding an antibody molecule which binds to tau, wherein the AAV capsid variant comprises:
   (a) the amino acid sequence of any of SEQ ID NO: 3648-3659 or a sequence provided in Table 20;
   (b) an amino acid sequence comprising at least 5, 6, 7, 8, or 9 consecutive amino acids from the amino acid sequence of any of SEQ ID NO: 3648-3659 or a sequence provided in Table 20;
   (c) an amino acid sequence comprising at least one, two, or three but no more than four modifications, e.g., substitutions, (e.g., conservative substitutions), insertions, or deletions, relative to the amino acid sequence of any of 3648-3659 or a sequence provided in Table 20; or
   (d) an amino acid sequence comprising at least one, two, or three but no more than four different amino acids relative to the amino acid sequence of any of 3648-3659 or a sequence provided in Table 20;
   optionally wherein the capsid variant comprises the amino acid sequence of SEQ ID NO: 138, or an amino acid sequence with at least 95% sequence identity thereto.
3. The isolated AAV particle of any one of embodiments 1 or 2, wherein the AAV capsid variant comprises:
   (a) the amino acid sequence of PLNGAVHLY (SEQ ID NO: 3648), or at least 5, 6, 7, or 8 consecutive amino acids from the amino acid sequence of PLNGAVHLY (SEQ ID NO: 3648);
   (b) the amino acid sequence of IVMNSLK (SEQ ID NO: 3651), or at least 4, 5, or 6 consecutive amino acids from the amino acid sequence of IVMNSLK (SEQ ID NO: 3651); or
   (c) the amino acid sequence of YSTDVRM (SEQ ID NO: 3650), YSTDERM (SEQ ID NO: 3657), or YSTDERK (SEQ ID NO: 3658), or at least 4, 5, or 6 consecutive amino acids from the amino acid sequence of YSTDVRM (SEQ ID NO: 3650), YSTDERM (SEQ ID NO: 3657), or YSTDERK (SEQ ID NO: 3658).
4. The isolated AAV particle of embodiment 2 or 3, wherein:
   (i) the 5 consecutive amino acids comprise PLNGA (SEQ ID NO: 3679);
   (ii) the 6 consecutive amino acids comprise PLNGAV (SEQ ID NO: 3680);
   (iii) the 7 consecutive amino acids comprise PLNGAVH (SEQ ID NO: 3681);
   (iv) the 8 consecutive amino acids comprise PLNGAVHL (SEQ ID NO: 3682); and/or
   (v) the 9 consecutive amino acids comprise PLNGAVHLY (SEQ ID NO: 3648);
   optionally wherein the amino acid sequence of (i), (ii), (iii), (iv), or (v) is present immediately subsequent to position 586, relative to a reference sequence numbered according to the amino acid sequence of SEQ ID NO: 138.
5. The isolated AAV particle of any one of embodiments 2-4, wherein:
   (i) the 5 consecutive amino acids comprise IVMNS (SEQ ID NO: 3694);
   (ii) the 6 consecutive amino acids comprise IVMNSL (SEQ ID NO: 3695); and/or
   (iii) the 7 consecutive amino acids comprise IVMNSLK (SEQ ID NO: 3651), optionally wherein the amino acid sequence of (i), (ii), or (iii) is present immediately subsequent to position 588, relative to a reference sequence numbered according to the amino acid sequence of SEQ ID NO: 138.
6. The isolated AAV particle of any one of embodiments 1-5, wherein the AAV capsid variant comprises:
   (i) the amino acid sequence of PLNGAVHLY (SEQ ID NO: 3648), wherein the amino acid sequence of PLNGAVHLY (SEQ ID NO: 3648) is present immediately subsequent to position 586, relative to a reference sequence numbered according to the amino acid sequence of SEQ ID NO: 138;
   (ii) the amino acid sequence of GGTLAVVSL (SEQ ID NO: 3654), wherein the amino acid sequence of GGTLAVVSL (SEQ ID NO: 3654) is present immediately subsequent to position 586, relative to a reference sequence numbered according to the amino acid sequence of SEQ ID NO: 138;
   (iii) the amino acid sequence of IVMNSLK (SEQ ID NO: 3651), wherein the amino acid sequence of IVMNSLK (SEQ ID NO: 3651) is present immediately subsequent to position 588, relative to a reference sequence numbered according to the amino acid sequence of SEQ ID NO: 138; or
   (iv) the amino acid sequence of any of SEQ ID NOs: 3649, 3650, 3652, 3653, or 3655-3659, wherein the amino acid sequence of any of the aforesaid sequences is present immediately subsequent to position 589, relative to a reference sequence numbered according to the amino acid sequence of SEQ ID NO: 138.
7. The isolated AAV particle of any one of embodiments 1-6, wherein:
   (i) the AAV capsid variant comprises an amino acid sequence encoded by the nucleotide sequence of any one of SEQ ID NOs: 3660-3671, or a nucleotide sequence having at least 80%, 85%, 90%, 92%, 95%, 96%, 97%, 98%, or 99% sequence identity thereto;
   (ii) the AAV capsid variant comprises an amino acid sequence encoded by a nucleotide sequence comprising at least one, two, three, four, five, six, or seven modifications, e.g., substitutions (e.g., conservative substitutions), but no more than ten modifications of the nucleotide sequences of any of SEQ ID NOs: 3660-3671;
   (iii) the AAV capsid variant comprises an amino acid sequence encoded by a nucleotide sequence comprising at least one, two, three, four, five, six, or seven but no more than ten different nucleotides relative to the nucleotide sequences of any of SEQ ID NOs: 3660-3671;
   (iv) the nucleotide sequence encoding the AAV capsid variant comprises the nucleotide sequence of any one of SEQ ID NOs: 3660-3671, or a nucleotide sequence having at least 80%, 85%, 90%, 92%, 95%, 96%, 97%, 98%, or 99% sequence identity thereto; and/or
   (v) the nucleotide sequence encoding the AAV capsid variant comprises a nucleotide sequence comprising at least one, two, three, four, five, six, or seven modifications, e.g., substitutions (e.g., conservative substitutions), but no more than ten modifications of the nucleotide sequence of any of SEQ ID NOs: 3660-3671.
8. The isolated AAV particle of any one of embodiments 1-5, wherein the AAV capsid variant comprises:
   (i) the AAV capsid variant comprises the amino acid sequence of any one of SEQ ID NOs: 3636-3647, or an amino acid sequence with at least 90% (e.g., at least 95, 96, 97, 98, or 99%) sequence identity thereto; or
   (ii) the nucleotide sequence encoding the AAV capsid variant comprises the nucleotide sequence of any one of SEQ ID NOs: 3623-3635, or a nucleotide sequence with at least 90% (e.g., at least 95, 96, 97, 98, or 99%) sequence identity thereto.
9. An isolated, e.g., recombinant, AAV particle comprising an AAV capsid variant and a nucleic acid comprising a transgene encoding an antibody molecule which binds to tau (e.g., human tau), wherein the AAV capsid variant comprises:
   (i) the amino acid sequence of PLNGAVHLY (SEQ ID NO: 3648);
   (ii) an amino acid sequence comprising at least 5, 6, 7, 8, or 9 consecutive amino acids from the amino acid sequence of PLNGAVHLY (SEQ ID NO: 3648);
   (iii) an amino acid sequence comprising at least one, two, or three but no more than four modifications, e.g., substitutions (e.g., conservative substitutions), insertions, or deletions, relative to the amino acid sequence of PLNGAVHLY (SEQ ID NO: 3648); or
   (iv) an amino acid sequence comprising at least one, two, or three but no more than four different amino acids relative to the amino acid sequence of PLNGAVHLY (SEQ ID NO: 3648);
   optionally wherein the capsid variant comprises the amino acid sequence of SEQ ID NO: 138, or an amino acid sequence with at least 95% sequence identity thereto.
10. The isolated AAV particle of embodiment 9, wherein the amino acid sequence of (i), (ii), or (iii) is present in loop VIII, relative to a reference sequence of SEQ ID NO: 138.
11. The isolated AAV particle of any one of embodiments 9 or 10, wherein the amino acid sequence is present immediately subsequent to position 586, relative to a reference sequence numbered according to the amino acid sequence of SEQ ID NO: 138.
12. The isolated AAV particle of any one of embodiments 1-11, wherein the AAV capsid variant:
   (i) is enriched at least about 300 or 400-fold compared, in the brain compared to a reference sequence of SEQ ID NO: 138, e.g., when measured by an assay as described in Example 1;
   (ii) transduces a brain region, e.g., selected from dentate nucleus, cerebellar cortex, cerebral cortex, brain stem, hippocampus, thalamus and putamen, wherein the level of transduction is at least 500, 1,000, 2,000, 5,000, or 10,000-fold greater as compared to a reference sequence of SEQ ID NO: 138, e.g., when measured by an assay, e.g., an immunohistochemistry assay, a qRT-PCR, or a RT-ddPCR assay, e.g., as described in Example 2;
   (iii) delivers an increased level of a payload to a brain region, optionally wherein the level of the payload is increased by at least 500, 1,000, 2,000, 5,000, or 10,000-fold, as compared to a reference sequence of SEQ ID NO: 138, e.g., when measured by an assay, e.g., a qRT-PCR or a RT-ddPCR assay (e.g., as described in Example 2), optionally wherein the brain region comprises a frontal cortex, sensory cortex, motor cortex, putamen, thalamus, cerebellar cortex, dentate nucleus, caudate, and/or hippocampus;
   (iv) delivers an increased level of a payload to a spinal cord region, optionally wherein the level of the payload is increased by at least 50, 100, 200, 300, 400, 500, 600, 700, 800 or 900-fold, as compared to a reference sequence of SEQ ID NO: 138, e.g., when measured by an assay, e.g., a qRT-PCR assay (e.g., as described in Example 2), optionally wherein the spinal cord region comprises a cervical, thoracic, and/or lumbar region; and/or
   (v) delivers an increased level of viral genomes to a brain region, optionally wherein the level of viral genomes is increased by at least 5, 10, 20, 30, 40 or 50-fold, as compared to a reference sequence of SEQ ID NO: 138, e.g., when measured by an assay, e.g., a qRT-PCR or a RT-ddPCR assay (e.g., as described in Example 2), optionally wherein the brain region comprises a frontal cortex, sensory cortex, motor cortex, putamen, thalamus, cerebellar cortex, dentate nucleus, caudate, and/or hippocampus.
13. The isolated AAV particle of any one of embodiments 1-8 or 10, wherein:
   (i) the AAV capsid variant has an increased tropism for a muscle cell or tissue, e.g., a heart tissue, relative to the tropism of a reference sequence comprising the amino acid sequence of SEQ ID NO: 138; and/or
   (ii) the AAV capsid variant delivers an increased level of a payload to a muscle region, optionally wherein the level of the payload is increased by at least 10, 15, 20, 30, or 40-fold, as compared to a reference sequence of SEQ ID NO: 138, e.g., when measured by an assay, e.g., an IHC assay or a RT-ddPCR assay (e.g., as described in Example 2), optionally wherein the muscle region comprises a heart muscle (e.g., a heart atrium muscle region or a heart ventricle muscle region), quadriceps muscle, and/or a diaphragm muscle region.
14. The isolated AAV particle of any one of embodiments 1-13, wherein the AAV capsid variant comprises:
   (i) a VP1 protein, a VP2 protein, a VP3 protein, or a combination thereof;
   (ii) the amino acid sequence corresponding to positions 138-743, e.g., a VP2, of any one of SEQ ID NOs: 3636-3647, or a sequence with at least 90% (e.g., at least about 95, 96, 97, 98, or 99%) sequence identity thereto;
   (iii) the amino acid sequence corresponding to positions 203-743, e.g., a VP3, of any one of SEQ ID NOs: 3636-3647, or a sequence with at least 90% (e.g., at least about 95, 96, 97, 98, or 99%) sequence identity thereto;
   (iv) the amino acid sequence of any one of SEQ ID NOs: 3636-3647, or an amino acid sequence with at least 90% (e.g., at least about 95, 96, 97, 98, or 99%) sequence identity thereto;
   (v) an amino acid sequence having at least one, two or three modifications, but not more than 30, 20 or 10 modifications, e.g., substitutions (e.g., conservative substitutions), of the amino acid sequence of any one of SEQ ID NOs: 3636-3647;
   (vi) an amino acid sequence having at least one, two or three, but no more than 30, 20 or 10 different amino acids relative to the amino acid sequence of any one of SEQ ID NOs: 3636-3647; and/or
   (vii) an amino acid sequence encoded by the nucleotide sequence of any one of SEQ ID NOs: 3623-3635, or a nucleotide sequence with at least 90% (e.g., at least about 95, 96, 97, 98, or 99%) sequence identity thereto.
15. An isolated, e.g., recombinant, AAV particle comprising an AAV capsid variant and a nucleic acid comprising a transgene encoding an antibody molecule which binds to tau (e.g., human tau), wherein the AAV capsid variant comprises an amino sequence comprising the following formula: [N1]−[N2], wherein:
   (i) [N1] comprises X1, X2, X3, X4, and X5, wherein:
      (a) position X1 is: P, Q, A, H, K, L, R, S, or T;
      (b) position X2 is: L, I, V, H, or R;
      (c) position X3 is: N, D, I, K, or Y;
      (d) position X4 is: G, A, C, R, or S; and
      (e) position X5 is: A, S, T, G, C, D, N, Q, V, or Y; and
   (ii) [N2] comprises the amino acid sequence of VHLY (SEQ ID NO: 4680), VHIY (SEQ ID NO: 4681), VHVY (SEQ ID NO: 4682), or VHHY (SEQ ID NO: 4683); and/or
   an amino acid modification, e.g., a conservative substitution, of any of the aforesaid amino acids in (i) and/or (ii);
   optionally wherein the AAV capsid variant further comprises:
   (i) one, two, or all of an amino acid other than T at position 593 (e.g., V, L, R, S, A, C, I, K, M, N, P, or Q), an amino acid other than G at position 594 (e.g., S, A, T, M, V, Q, L, H, I, K, N, P, R, or Y), and/or an amino acid other than W at position 595 (e.g., S, P, G, A, Q, L, M, K, C, E, F, H, R, T, V, or Y), relative to a reference sequence numbered according to the amino acid sequence of SEQ ID NO: 138; and/or
   (ii) one, two, or all of an amino acid other than V at position 596 (e.g., D, F, G, L, A, E, or I), an amino acid other than Q at position 597 (e.g., P, K, R, H, E, or L), and/or an amino acid other than N at position 598 (e.g., T, K, H, D, Y, S, I, or P), relative to a reference sequence numbered according to the amino acid sequence of SEQ ID NO: 138.
16. An isolated, e.g., recombinant, AAV particle comprising an AAV capsid variant and a nucleic acid comprising a transgene encoding an antibody molecule which binds to tau (e.g., human tau), wherein the AAV capsid variant comprises one, two, three, four, or all of:
   (i) an [N1], wherein [N1] is or comprises: PLNGA (SEQ ID NO: 3679), SLNGA (SEQ ID NO: 4684), QLNGA (SEQ ID NO: 4685), ALNGA (SEQ ID NO: 4686), PLNGS (SEQ ID NO: 4687), PVNGA (SEQ ID NO: 4688), PLNGG (SEQ ID NO: 4689), PLNGT (SEQ ID NO: 4690), PLDGA (SEQ ID NO: 4691), QLNGS (SEQ ID NO: 4692), PLNGN (SEQ ID NO: 4693), SLDGA (SEQ ID NO: 4694), HLNGA (SEQ ID NO: 4695), ALNGT (SEQ ID NO: 4696), PINGA (SEQ ID NO: 4697), ALDGA (SEQ ID NO: 4698), PLNCA (SEQ ID NO: 4699), PLNGQ (SEQ ID NO: 4700), PLDSA (SEQ ID NO: 4701), RLDGA (SEQ ID NO: 4702), QLNGN (SEQ ID NO: 4703), PLNGY (SEQ ID NO: 4704), PLDSS (SEQ ID NO: 4705), PLNGC (SEQ ID NO: 4706), PLYGA (SEQ ID NO: 4707), TLNGA (SEQ ID NO: 4708), PVDGA (SEQ ID NO: 4709), PLKGA (SEQ ID NO: 4710), PLNGD (SEQ ID NO: 4711), KLDGA (SEQ ID NO: 4712), PHNGA (SEQ ID NO: 4713), PLNGV (SEQ ID NO: 4714), PLNAA (SEQ ID NO: 4715), QLNGY (SEQ ID NO: 4716), PLDGS (SEQ ID NO: 4717), LLNGA (SEQ ID NO: 4718), PLNRA (SEQ ID NO: 4719), PLIGA (SEQ ID NO: 4720), PRNGA (SEQ ID NO: 4721), or ALNGS (SEQ ID NO: 4722);
   (ii) an [N2] wherein [N2] is or comprises: VHLY (SEQ ID NO: 4680), VHVY (SEQ ID NO: 4682), VPLY (SEQ ID NO: 4723), VNLY (SEQ ID NO: 4724), VHRY (SEQ ID NO: 4725), VHIY (SEQ ID NO: 4681), VHHY (SEQ ID NO: 4683), FHLY (SEQ ID NO: 4726), LHLY (SEQ ID NO: 4727), DHLY (SEQ ID NO: 4728), VQLY (SEQ ID NO: 4729), IHLY (SEQ ID NO: 4730), VDLY (SEQ ID NO: 4731), AHLY (SEQ ID NO: 4732), VLLY (SEQ ID NO: 4733), GHLY (SEQ ID NO: 4734), VRLY (SEQ ID NO: 4735), or VYLY (SEQ ID NO: 4736);
   (iii) an [N3] wherein [N3] is or comprises: AQAQ (SEQ ID NO: 4737), SQAQ (SEQ ID NO: 4738), AQPQ (SEQ ID NO: 4739), AQSQ (SEQ ID NO: 4740), AKAQ (SEQ ID NO: 4741), AHAQ (SEQ ID NO: 4742), AQAP (SEQ ID NO: 4743), DQAQ (SEQ ID NO: 4744), APAQ (SEQ ID NO: 4745), AQAK (SEQ ID NO: 4746), AQAH (SEQ ID NO: 4747), AQEQ (SEQ ID NO: 4748), ALAQ (SEQ ID NO: 4749), ARAQ (SEQ ID NO: 4750), or TQAQ (SEQ ID NO: 4751);
   (iv) an [N4] wherein [N4] is or comprises: TGW, TGL, TGS, TGG, TAW, TGR, TAS, LSS, TSS, SSL, SSS, TLS, TVS, VSS, TSP, VSP, TMS, LSP, VAS, TAL, TTS, TLP, VLP, RGW, LSG, LAS, SSP, LLP, STS, TSA, TTP, SAL, LGS, VTP, VSA, IGW, TGF, LTP, TLA, LSA, TVG, TAP, TMP, TSL, VQS, SSM, SLP, VSQ, RSS, TST, VMS, TTA, TQP, LST, LAP, TVA, RLS, TGY, TSG, TAG, VMP, TSQ, TMA, VGS, TSW, TGV, TGT, TLG, LMP, VQP, TGM, SMS, SQL, IGS, RSV, TAA, STP, LSQ, TAQ, TGP, ASP, VSG, SAP, TLQ, LQP, TAT, TGQ, ATS, IGG, VAA, TSM, TVW, TAM, TGA, VAT, QSP, TQA, VQA, RSP, LAT, VAQ, LAA, RST, RTL, LGT, LMS, LGP, RTS, SQP, VLG, SVS, TMQ, SAV, LAG, SGP, TNS, RLT, TTQ, SAA, TSV, RLG, RAS, STQ, CSP, SAG, ALP, VTS, ISP, SVG, LTS, TTT, RSG, TQL, LNP, TVQ, IAS, LAQ, LSR, LSN, TTG, TSN, SMA, TKS, SVA, TQQ, VQQ, RLP, SAM, TAV, TQW, SSR, TQT, VNS, RSA, LMG, RQS, LVG, VTA, RTT, SMG, VMA, TKP, SAQ, NSP, ATP, VAG, RGS, VKP, RMS, NLP, NAL, RTP, RQL, VQG, VTG, VST, NAS, RVE, ATG, AMS, RNS, VMQ, SMQ, LQQ, TMG, LGQ, TSH, AAP, RSQ, TYS, ITP, VAK, TQM, TKA, SQQ, ISG, VSR, RTA, RML, SQM, VAN, CTP, ISS, AGP, TAK, RTG, LHP, TMT, AQP, QAP, RQP, LKS, NTT, TSK, RYS, KSS, NTP, VGG, IAA, LMA, MAP, VHP, VLS, LAN, ATQ, TNA, TAN, VSN, AAA, AVG, LTA, SAN, RAG, RQG, TLR, LSH, SAF, RAA, IQP, ILG, VNG, SVQ, LSK, TNG, RTQ, TMN, RGG, TTR, VRP, VKA, LAR, NQP, TMK, TYA, TQK, TTK, IAG, TQN, LAH, NTQ, RQQ, RAQ, TKQ, TQH, TNQ, LMQ, VNA, VQT, TQR, VGK, VKQ, IQS, LQR, TMM, VGN, RIG, SAK, RIA, VQN, NVQ, RIP, NAQ, NMQ, TPS, LTN, VTK, PGW, LPP, SPP, TPA, TGC, VPP, TPT, TPW, TPP, RPP, TPQ, TPR, TPG, VPA, VPQ, RPG, KGW, TRW, TAR, IPP, RSL, LVP, KGS, VAP, KGG, KAW, PGS, TRL, or AGW; and/or
   (v) an [N5] wherein [N5] is or comprises: VQN, VKN, VQT, VQK, DQN, VQH, GQN, VQI, VHN, FQN, LQN, VLN, VRN, VQS, VQY, AQN, VEN, VQD, VPN, IQN, VKK, DKN, VKT, VQP, EQN, GQT, FQK, GHN, or VPH; and/or
   wherein the AAV capsid variant comprises an amino acid modification, e.g., a conservative substitution, of any of the aforesaid amino acids in (i)-(v).
17. The isolated AAV particle of embodiment 15, wherein:
   (a) position X1 is: P, Q, A, S, or T;
   (b) position X2 is L or I;
   (c) position X3 is N or D;
   (d) position X4 is G or S; and/or
   (e) position X5 is: A, S, G, T, or N.
18. The isolated AAV particle of embodiment 15 or 17, wherein [N1] comprises AL, PI, PL, QL, SL, TL, LN, LD, IN, NG, DG, DS, GA, SA, SS, GG, GN, GS, or GT.
19. The isolated AAV particle of any one of embodiments 15, 17, or 18, wherein [N1] comprises ALD, ALN, PIN, PLD, PLN, QLN, SLD, SLN, TLN, LNG, LDG, ING, LDS, NGA, DGA, DSA, DSS, NGG, NGN, NGS, NGT.
20. The isolated AAV particle of any one of embodiments 15 or 17-19, wherein [N1] comprises ALDG (SEQ ID NO: 4762), ALNG (SEQ ID NO: 4758), PING (SEQ ID NO: 4763), PLDG (SEQ ID NO: 4764), PLDS (SEQ ID NO: 4765), PLNG (SEQ ID NO: 3678), QLNG (SEQ ID NO: 4766), SLDG (SEQ ID NO: 4767), SLNG (SEQ ID NO: 4756), or TLNG (SEQ ID NO: 4754).
21. The isolated AAV particle of any one of embodiments 15-20, wherein [N1] is or comprises PLNGA (SEQ ID NO: 3679), SLNGA (SEQ ID NO: 4684), QLNGA (SEQ ID NO: 4685), ALNGA (SEQ ID NO: 4686), PLNGS (SEQ ID NO: 4687), PVNGA (SEQ ID NO: 4688), PLNGG (SEQ ID NO: 4689), PLNGT (SEQ ID NO: 4690), PLDGA (SEQ ID NO: 4691), QLNGS (SEQ ID NO: 4692), PLNGN (SEQ ID NO: 4693), SLDGA (SEQ ID NO: 4694), HLNGA (SEQ ID NO: 4695), ALNGT (SEQ ID NO: 4696), PINGA (SEQ ID NO: 4697), ALDGA (SEQ ID NO: 4698), PLNCA (SEQ ID NO: 4699), PLNGQ (SEQ ID NO: 4700), PLDSA (SEQ ID NO: 4701), RLDGA (SEQ ID NO: 4702), QLNGN (SEQ ID NO: 4703), PLNGY (SEQ ID NO: 4704), PLDSS (SEQ ID NO: 4705), PLNGC (SEQ ID NO: 4706), PLYGA (SEQ ID NO: 4707), TLNGA (SEQ ID NO: 4708), PVDGA (SEQ ID NO: 4709), PLKGA (SEQ ID NO: 4710), PLNGD (SEQ ID NO: 4711), KLDGA (SEQ ID NO: 4712), PHNGA (SEQ ID NO: 4713), PLNGV (SEQ ID NO: 4714), PLNAA (SEQ ID NO: 4715), QLNGY (SEQ ID NO: 4716), PLDGS (SEQ ID NO: 4717), LLNGA (SEQ ID NO: 4718), PLNRA (SEQ ID NO: 4719), PLIGA (SEQ ID NO: 4720), PRNGA (SEQ ID NO: 4721), or ALNGS (SEQ ID NO: 4722).
22. The isolated AAV particle of embodiment 15-21, wherein [N1] is or comprises PLNGA (SEQ ID NO: 3679), ALDGA (SEQ ID NO: 4698), ALNGA (SEQ ID NO: 4686), PINGA (SEQ ID NO: 4697), PLDGA (SEQ ID NO: 4691), PLDSA (SEQ ID NO: 4701), PLDSS (SEQ ID NO: 4705), PLNGG (SEQ ID NO: 4689), PLNGN (SEQ ID NO: 4693), PLNGS (SEQ ID NO: 4687), PLNGT (SEQ ID NO: 4690), QLNGA (SEQ ID NO: 4685), SLDGA (SEQ ID NO: 4694), SLNGA (SEQ ID NO: 4684), or TLNGA (SEQ ID NO: 4708).
23. The isolated AAV particle of embodiment 15-22, wherein [N1] is or comprises PLNGA (SEQ ID NO: 3679).
24. The isolated AAV particle of any one of embodiments 15 or 17-18, or 20-23, wherein [N1]-[N2] comprises:

(i)
(SEQ ID NO: 4768)
LDGAVHLY,
(SEQ ID NO: 4769)
LNGAVHLY,
(SEQ ID NO: 4770)
INGAVHLY,
(SEQ ID NO: 4771)
LDSAVHLY,
(SEQ ID NO: 4772)
LDSSVHLY,
(SEQ ID NO: 4773)
LNGGVHLY,
(SEQ ID NO: 4774)
LNGNVHLY,
(SEQ ID NO: 4775)
LNGSVHLY,
(SEQ ID NO: 4776)
LNGTVHLY,
(SEQ ID NO: 4777)
LNGAVHIY,
(SEQ ID NO: 4778)
LDGAVHVY,
or
(SEQ ID NO: 4779)
LNGAVHHY;

(ii) an amino acid sequence comprising any portion of an amino acid sequence in (i), e.g., any 2, 4, 5, 6, or 7 amino acids, e.g., consecutive amino acids, thereof;
(iii) an amino acid sequence comprising at least one, two, or three but no more than four modifications, e.g., substitutions (e.g., conservative substitutions), insertions, or deletions, relative to any of the amino acid sequences in (i); or
(iv) an amino acid sequence comprising at least one, two, or three but no more than four different amino acids, relative to any one of the amino acid sequences in (i).
25. The isolated AAV particle of any one of embodiments 15-24, wherein [N1]-[N2] is or comprises:

(i)
(SEQ ID NO: 3648)
PLNGAVHLY,
(SEQ ID NO: 4780)
ALDGAVHLY,
(SEQ ID NO: 4781)
ALNGAVHLY,
(SEQ ID NO: 4782)
PINGAVHLY,
(SEQ ID NO: 4783)
PLDGAVHLY,
(SEQ ID NO: 4784)
PLDSAVHLY,
(SEQ ID NO: 4785)
PLDSSVHLY,
(SEQ ID NO: 4786)
PLNGGVHLY,
(SEQ ID NO: 4787)
PLNGNVHLY,
(SEQ ID NO: 4788)
PLNGSVHLY,
(SEQ ID NO: 4789)
PLNGTVHLY,
(SEQ ID NO: 4790)
QLNGAVHLY,
(SEQ ID NO: 4791)
SLDGAVHLY,
(SEQ ID NO: 4792)
SLNGAVHLY,
(SEQ ID NO: 4793)
TLNGAVHLY,
(SEQ ID NO: 4794)
PLNGAVHIY,
(SEQ ID NO: 4795)
PLDGAVHVY,
or
(SEQ ID NO: 4796)
PLNGAVHHY;

(ii) an amino acid sequence comprising any portion of an amino acid sequence in (i), e.g., any 2, 3, 4, 5, 6, 7, or 8 amino acids, e.g., consecutive amino acids, thereof;
(iii) an amino acid sequence comprising at least one, two, or three but no more than four modifications, e.g., substitutions (e.g., conservative substitutions), insertions, or deletions, relative to any of the amino acid sequences in (i); or
(iv) an amino acid sequence comprising at least one, two, or three but no more than four different amino acids, relative to any one of the amino acid sequences in (i).
26. The isolated AAV particle of any one of embodiments 15-25, wherein [N1]-[N2] is or comprises PLNGAVHLY (SEQ ID NO: 3648).
27. The isolated AAV particle of any one of embodiments 15-26, wherein the AAV capsid variant further comprises one, two, three, or all of an amino acid other than A at position 589 (e.g., D, S, or T), an amino acid other than Q at position 590 (e.g., K, H, L, P, or R), an amino acid other than A at position 591 (e.g., P, E, or R), and/or an amino acid other than Q at position 592 (e.g., H, K, or P).
28. The isolated AAV particle of any one of embodiments 15-27, wherein the AAV capsid variant further comprises one, two, three, or all of an amino acid other than A at position 596 (e.g., D, S, or T), an amino acid other than Q at position 597 (e.g., K, H, L, P, or R), an amino acid other than A at position 598 (e.g., P, E, or R), and/or an amino acid other than Q at position 599 (e.g., H, K, or P), numbered according to the amino acid sequence of SEQ ID NO: 5, 8, or 3636.
29. The isolated AAV particle of any one of embodiments 15-28, wherein the AAV capsid variant further comprises
   (i) A at position 589, Q at position 590, A at position 591, and/or Q at position 592, numbered according to the amino acid sequence of SEQ ID NO: 138; or
   (ii) A at position 596, Q at position 597, A at position 598, and/or Q at position 599, numbered according to the amino acid sequence of SEQ ID NO: 5, 8, or 3636.
30. The isolated AAV particle of any one of embodiments 15 or 17-29, wherein the AAV capsid variant further comprises [N3], wherein [N3] comprises X6, X7, X8, and X9, wherein:
   (a) position X6 is: A, D, S, or T;
   (b) position X7 is: Q, K, H, L, P, or R;
   (c) position X8 is: A, P, E, or R; and
   (d) position X9 is: Q, H, K, or P; and/or an amino acid modification, e.g., a conservative substitution, of any of the aforesaid amino acids in (a)-(d).
31. The isolated AAV particle of embodiment 30, wherein:
   (a) position X6 is: A, D, S;
   (b) position X7 is Q or K;
   (c) position X8 is A or P; and/or
   (d) position X9 is Q.
32. The isolated AAV particle of embodiment 30 or 31, wherein [N3] comprises AQ, SQ, AK, DQ, PQ, QA, QP, or KA.
33. The isolated AAV particle of any one of embodiments 30-32, wherein [N3] comprises AQA, AQP, SQA, AKA, DQA, QAQ, QPQ, or KAQ.
34. The isolated AAV particle of any one of embodiments 30-33, wherein [N3] is or comprises AQAQ (SEQ ID NO: 4737), SQAQ (SEQ ID NO: 4738), AQPQ (SEQ ID NO: 4739), AQSQ (SEQ ID NO: 4740), AKAQ (SEQ ID NO: 4741), AHAQ (SEQ ID NO: 4742), AQAP (SEQ ID NO: 4743), DQAQ (SEQ ID NO: 4744), APAQ (SEQ ID NO: 4745), AQAK (SEQ ID NO: 4746), AQAH (SEQ ID NO: 4747), AQEQ (SEQ ID NO: 4748), ALAQ (SEQ ID NO: 4749), ARAQ (SEQ ID NO: 4750), or TQAQ (SEQ ID NO: 4751).
35. The isolated AAV particle of any one of embodiments 16 or 30-34, wherein [N3] is or comprises AQAQ (SEQ ID NO: 4737), AQPQ (SEQ ID NO: 4739), SQAQ (SEQ ID NO: 4738), AKAQ (SEQ ID NO: 4741), or DQAQ (SEQ ID NO: 4744).
36. The isolated AAV particle of any one of embodiments 16 or 30-35, wherein [N3] is or comprises AQAQ (SEQ ID NO: 4737).
37. The isolated AAV particle of any one of embodiments 16 or 30-36, wherein [N2]-[N3] comprises:

(i)
(SEQ ID NO: 4797)
VHLYAQAQ,
(SEQ ID NO: 4798)
VHLYAQPQ,
(SEQ ID NO: 4799)
VHLYSQAQ,
(SEQ ID NO: 4800)
VHLYAKAQ,
(SEQ ID NO: 4801)
VHLYDQAQ,
(SEQ ID NO: 4802)
VHIYAQAQ,
(SEQ ID NO: 4803)
VHVYAQAQ,
(SEQ ID NO: 4804)
VHHYAQAQ;

(ii) an amino acid sequence comprising any portion of an amino acid sequence in (i), e.g., any 2, 3, 4, 5, 6, or 7 amino acids, e.g., consecutive amino acids, thereof;
(iii) an amino acid sequence comprising at least one, two, or three but no more than four modifications, e.g., substitutions (e.g., conservative substitutions), insertions, or deletions, relative to any of the amino acid sequences in (i); or
(iv) an amino acid sequence comprising at least one, two, or three but no more than four different amino acids, relative to any one of the amino acid sequences in (i).
38. The isolated AAV particle of any one of embodiments 16 or 30-37, wherein [N2]-[N3] is or comprises VHLYAQAQ (SEQ ID NO: 4797).
39. The isolated AAV particle of any one of embodiments 30-38, wherein [N1]-[N2]-[N3] comprises:

(i)
(SEQ ID NO: 4805)
ALDGAVHLYAQ,
(SEQ ID NO: 4806)
ALNGAVHLYAQ,
(SEQ ID NO: 4807)
PINGAVHLYAQ,
(SEQ ID NO: 4808)
PLDGAVHLYAQ,
(SEQ ID NO: 4809)
PLDGAVHLYSQ,
(SEQ ID NO: 4810)
PLDSAVHLYAQ,
(SEQ ID NO: 4811)
PLDSSVHLYAQ,
(SEQ ID NO: 4812)
PLNGAVHLYAK,
(SEQ ID NO: 4813)
PLNGAVHLYAQ,
(SEQ ID NO: 4814)
PLNGAVHLYDQ,
(SEQ ID NO: 4815)
PLNGAVHLYSQ,
(SEQ ID NO: 4816)
PLNGGVHLYAQ,
(SEQ ID NO: 4817)
PLNGNVHLYAQ,
(SEQ ID NO: 4818)
PLNGSVHLYAQ,
(SEQ ID NO: 4819)
PLNGTVHLYAQ,
(SEQ ID NO: 4820)
QLNGAVHLYAQ,
(SEQ ID NO: 4821)
SLDGAVHLYAQ,
(SEQ ID NO: 4822)
SLNGAVHLYAQ,
(SEQ ID NO: 4823)
TLNGAVHLYAQ,
(SEQ ID NO: 4824)
PLNGAVHIYAQ,
(SEQ ID NO: 4825)
PLDGAVHVYAQ,
or
(SEQ ID NO: 4826)
PLNGAVHHYAQ;

(ii) an amino acid sequence comprising any portion of an amino acid sequence in (i), e.g., any 2, 3, 4, 5, 6, 7, 8, 9, or 10 amino acids, e.g., consecutive amino acids, thereof;
(iii) an amino acid sequence comprising at least one, two, or three but no more than four modifications, e.g., substitutions (e.g., conservative substitutions), insertions, or deletions, relative to any of the amino acid sequences in (i); or
(iv) an amino acid sequence comprising at least one, two, or three but no more than four different amino acids, relative to any one of the amino acid sequences in (i).
40. The isolated AAV particle of any one of embodiments 16 or 30-39, wherein [N1]-[N2]-[N3] is or comprises:

(i)
(SEQ ID NO: 4836)
PLNGAVHLYAQAQ,
(SEQ ID NO: 4827)
ALDGAVHLYAQAQ,
(SEQ ID NO: 4828)
ALNGAVHLYAQAQ,
(SEQ ID NO: 4829)
PINGAVHLYAQAQ,
(SEQ ID NO: 4830)
PLDGAVHLYAQAQ,
(SEQ ID NO: 4831)
PLDGAVHLYAQPQ,
(SEQ ID NO: 4832)
PLDGAVHLYSQAQ,
(SEQ ID NO: 4833)
PLDSAVHLYAQAQ,
(SEQ ID NO: 4834)
PLDSSVHLYAQAQ,
(SEQ ID NO: 4835)
PLNGAVHLYAKAQ,
(SEQ ID NO: 4837)
PLNGAVHLYAQPQ,
(SEQ ID NO: 4838)
PLNGAVHLYDQAQ,
(SEQ ID NO: 4839)
PLNGAVHLYSQAQ,
(SEQ ID NO: 4840)
PLNGGVHLYAQAQ,
(SEQ ID NO: 4841)
PLNGNVHLYAQAQ,
(SEQ ID NO: 4842)
PLNGSVHLYAQAQ,
(SEQ ID NO: 4843)
PLNGTVHLYAQAQ,
(SEQ ID NO: 4844)
QLNGAVHLYAQAQ,
(SEQ ID NO: 4845)
SLDGAVHLYAQAQ,
(SEQ ID NO: 4846)
SLNGAVHLYAQAQ,
(SEQ ID NO: 4847)
TLNGAVHLYAQAQ,
(SEQ ID NO: 4848)
PLNGAVHIYAQAQ,
(SEQ ID NO: 4849)
PLDGAVHVYAQAQ,
or
(SEQ ID NO: 4850)
PLNGAVHHYAQAQ;

(ii) an amino acid sequence comprising any portion of an amino acid sequence in (i), e.g., any 2, 3, 4, 5, 6, 7, 8, 9, 10, 11, or 12 amino acids, e.g., consecutive amino acids, thereof;
(iii) an amino acid sequence comprising at least one, two, or three but no more than four modifications, e.g., substitutions (e.g., conservative substitutions), insertions, or deletions, relative to any of the amino acid sequences in (i); or
(iv) an amino acid sequence comprising at least one, two, or three but no more than four different amino acids, relative to any one of the amino acid sequences in (i).
41. The isolated AAV particle of any one of embodiments 16 or 30-40, wherein [N1]-[N2]-[N3] is or comprises PLNGAVHLYAQAQ (SEQ ID NO: 4836).
42. The isolated AAV particle of any one of embodiments 15-41, wherein the AAV capsid variant further comprises one, two, or all of an amino acid other than T at position 593 (e.g., V, L, R, S, A, C, I, K, M, N, P, or Q), an amino acid other than G at position 594 (e.g., S, A, T, M, V, Q, L, H, I, K, N, P, R, or Y), and/or an amino acid other than W at position 595 (e.g., S, P, G, A, Q, L, M, K, C, E, F, H, R, T, V, or Y), relative to a reference sequence numbered according to the amino acid sequence of SEQ ID NO: 138.
43. The isolated AAV particle of any one of embodiments 15-42, wherein the AAV capsid variant further comprises one, two, or all of an amino acid other than T at position 600 (e.g., V, L, R, S, A, C, I, K, M, N, P, or Q), an amino acid other than G at position 601 (e.g., S, A, T, M, V, Q, L, H, I, K, N, P, R, or Y), and/or an amino acid other than W at position 602 (e.g., S, P, G, A, Q, L, M, K, C, E, F, H, R, T, V, or Y), relative to a reference sequence numbered according to the amino acid sequence of SEQ ID NO: 5, 8, or 3636.
44. The isolated AAV particle of any one of embodiments 15-43, wherein the AAV capsid variant further comprises one, two, or all of:
   (i) the amino acid V, L, R, S, A, C, I, K, M, N, P, or Q (e.g., L) at position 593 numbered according to SEQ ID NO: 138 or at position 600 numbered according to SEQ ID NO: 5, 8, or 3636);
   (ii) the amino acid S, A, T, M, V, Q, L, H, I, K, N, P, R, or Y (e.g., S) at position 594 numbered according to SEQ ID NO: 138, or at position 601 numbered according to SEQ ID NO: 5, 8, or 3636; and/or
   (iii) the amino acid S, P, G, A, Q, L, M, K, C, E, F, H, R, T, V, or Y (e.g. P) at position 595 numbered according to SEQ ID NO: 138 or at position 602 numbered according to SEQ ID NO: 5, 8, or 3636).
45. The isolated AAV particle of any one of embodiments 15-44, wherein the AAV capsid variant further comprises:
   (i) the amino acid L at position 593 numbered according to SEQ ID NO: 138 or at position 600 numbered according to SEQ ID NO: 5, 8, or 3636;
   (ii) the amino acid S at position 594 numbered according to SEQ ID NO: 138, or at position 601 numbered according to SEQ ID NO: 5, 8, or 3636; and
   (iii) the amino acid P at position 595 numbered according to SEQ ID NO: 138 or at position 602 numbered according to SEQ ID NO: 5, 8, or 3636.
46. The isolated AAV particle of any one of embodiments 15-41, wherein the AAV capsid variant further comprises:
   (i) T at position 593, G at position 594, and/or W at position 595, numbered according to SEQ ID NO: 138;
   (ii) T at position 600, G at position 601, and/or W at position 602, numbered according to SEQ ID NO: 5, 8, or 3636.
47. The isolated AAV particle of any one of embodiments 15 or 17-46, wherein the AAV capsid variant further comprises [N4], wherein [N4] comprises X10, X11, and X12, wherein:
   (a) position X10 is: T, V, L, R, S, A, C, I, K, M, N, P, or Q;
   (b) position X11 is: G, S, A, T, M, V, Q, L, H, I, K, N, P, R, or Y; and
   (c) position X12 is: W, S, P, G, A, Q, L, M, K, C, E, F, H, R, T, V, or Y; and/or an amino acid modification, e.g., a conservative substitution, of any of the aforesaid amino acids in (a)-(c).
48. The isolated AAV particle of embodiment 47, wherein:
   (a) position X10 is: T, V, L, A, R, C, S, I, M, N, P, Q;
   (b) position X11 is: G, A, S, T, M, Q, V; and/or
   (c) position X12 is: P, S, W, G, A, Q, T, K, N, R, L, M, H, V, C, or E.
49. The isolated AAV particle of any one of embodiments 47 or 48, wherein [N4] comprises LS, TG, LA, LT, SA, SS, TL, TT, TS, TA, TV, VS, AA, AG, AS, AT, CS, CT, IA, IG, IL, IQ, IS, IT, LG, LH, LK, LM, LN, LQ, MA, NA, NM, NS, NT, NV, QA, RA, RG, RI, RL, RM, RN, RQ, RS, RT, RV, SG, SM, ST, SV, TK, TM, TN, TP, TQ, TR, VA, VG, VH, VK, VL, VM, VN, VQ, VR, VT, PG, LV, SP, GW, AP, GR, AL, AW, GG, GS, GP, QP, QS, AH, AN, AQ, AR, GQ, HP, KS, MG, MP, MQ, MS, NP, QQ, QR, SH, SK, SQ, SR, IP, VE, AK, AM, AV, GA, GC, GT, KA, KP, KQ, LP, MK, MN, MT, NQ, PP, QH, QK, QM, QN, QT, RW, SL, VW, GK, GN, NG, RP, SN, GL, or VP.
50. The isolated AAV particle of any one of embodiments 16 or 47-49, wherein [N4] is or comprises TGW, TGL, TGS, TGG, TAW, TGR, TAS, LSS, TSS, SSL, SSS, TLS, TVS, VSS, TSP, VSP, TMS, LSP, VAS, TAL, TTS, TLP, VLP, RGW, LSG, LAS, SSP, LLP, STS, TSA, TTP, SAL, LGS, VTP, VSA, IGW, TGF, LTP, TLA, LSA, TVG, TAP, TMP, TSL, VQS, SSM, SLP, VSQ, RSS, TST, VMS, TTA, TQP, LST, LAP, TVA, RLS, TGY, TSG, TAG, VMP, TSQ, TMA, VGS, TSW, TGV, TGT, TLG, LMP, VQP, TGM, SMS, SQL, IGS, RSV, TAA, STP, LSQ, TAQ, TGP, ASP, VSG, SAP, TLQ, LQP, TAT, TGQ, ATS, IGG, VAA, TSM, TVW, TAM, TGA, VAT, QSP, TQA, VQA, RSP, LAT, VAQ, LAA, RST, RTL, LGT, LMS, LGP, RTS, SQP, VLG, SVS, TMQ, SAV, LAG, SGP, TNS, RLT, TTQ, SAA, TSV, RLG, RAS, STQ, CSP, SAG, ALP, VTS, ISP, SVG, LTS, TTT, RSG, TQL, LNP, TVQ, IAS, LAQ, LSR, LSN, TTG, TSN, SMA, TKS, SVA, TQQ, VQQ, RLP, SAM, TAV, TQW, SSR, TQT, VNS, RSA, LMG, RQS, LVG, VTA, RTT, SMG, VMA, TKP, SAQ, NSP, ATP, VAG, RGS, VKP, RMS, NLP, NAL, RTP, RQL, VQG, VTG, VST, NAS, RVE, ATG, AMS, RNS, VMQ, SMQ, LQQ, TMG, LGQ, TSH, AAP, RSQ, TYS, ITP, VAK, TQM, TKA, SQQ, ISG, VSR, RTA, RML, SQM, VAN, CTP, ISS, AGP, TAK, RTG, LHP, TMT, AQP, QAP, RQP, LKS, NTT, TSK, RYS, KSS, NTP, VGG, IAA, LMA, MAP, VHP, VLS, LAN, ATQ, TNA, TAN, VSN, AAA, AVG, LTA, SAN, RAG, RQG, TLR, LSH, SAF, RAA, IQP, ILG, VNG, SVQ, LSK, TNG, RTQ, TMN, RGG, TTR, VRP, VKA, LAR, NQP, TMK, TYA, TQK, TTK, IAG, TQN, LAH, NTQ, RQQ, RAQ, TKQ, TQH, TNQ, LMQ, VNA, VQT, TQR, VGK, VKQ, IQS, LQR, TMM, VGN, RIG, SAK, RIA, VQN, NVQ, RIP, NAQ, NMQ, TPS, LTN, VTK, PGW, LPP, SPP, TPA, TGC, VPP, TPT, TPW, TPP, RPP, TPQ, TPR, TPG, VPA, VPQ, RPG, KGW, TRW, TAR, IPP, RSL, LVP, KGS, VAP, KGG, KAW, PGS, TRL, or AGW.
51. The isolated AAV particle of any one of embodiments 16 or 47-50, wherein [N4] is or comprises LSP, TGW, LAA, LTP, SAP, SSP, TGR, TLA, TTS, TSP, TAL, TAW, TGG, TGS, TVS, VSP, VSS, AAP AGP, ASP, ATP, CSP, CTP, IAA, IAG, IAS, IGG, IGS, ILG, IQP, IQS, ISG, ISP, ISS, ITP, LAG, LAH, LAN, LAP, LAQ, LAR, LAS, LAT, LGP, LGQ, LGS, LHP, LKS, LMA, LMG, LMP, LMQ, LMS, LNP, LQP, LQQ, LQR, LSH, LSK, LSQ, LSR, LST, LTA, LTN, LTS, MAP, NAQ, NAS, NMQ, NSP, NTP, NVQ, QAP, RAA, RAQ, RAS, RGG, RGS, RIA, RIG, RIP, RLG, RLS, RMS, RNS, RQP, RSA, RSG, RSP, RSQ, RSS, RST, RTA, RTG, RTL, RTS, RTT, RVE, SAA, SAK, SAM, SAQ, SGP, SMA, SMG, SMQ, SMS, STP, SVA, SVG, TAA, TAG, TAK, TAM, TAN, TAP, TAQ, TAS, TAT, TAV, TGA, TGC, TGP, TGT, TKA, TKP, TKQ, TKS, TLP, TLQ, TMA, TMG, TMK, TMN, TMP, TMQ, TMS, TMT, TNA, TNQ, TNS, TPP, TQH, TQK, TQM, TQN, TQP, TQQ, TQT, TRW, TSA, TSG, TSH, TSK, TSL, TSM, TSQ, TSS, TST, TSV, TTA, TTG, TTK, TTP, TTQ, TTT, TVA, TVG, TVQ, TVW, VAA, VAG, VAK, VAN, VAQ, VAS, VAT, VGG, VGK, VGN, VGS, VHP, VKA, VKP, VKQ, VLP, VLS, VMA, VMQ, VMS, VNA, VNG, VNS, VQA, VQN, VQP, VQQ, VQS, VQT, VRP, VSA, VSG, VSN, VSQ, VSR, VST, VTA, VTG, VTK, VTP, VTS, TGL, PGW, LSG, LSS, or LVP.
52. The isolated AAV particle of any one of embodiments 16 or 47-50, wherein [N4] is or comprises TGW.
53. The isolated AAV particle of any one of embodiments 16 or 47-50, wherein [N4] is or comprises LSP.
54. The isolated AAV particle of any one of embodiments 15-53, wherein the AAV capsid variant further comprises one, two, or all of an amino acid other than V at position 596 (e.g., D, F, G, L, A, E, or I), an amino acid other than Q at position 597 (e.g., K, R, H, E, L, or P), and/or an amino acid other than N at position 598 (e.g., T, K, H, D, Y, S, I, or P), relative to a reference sequence numbered according to the amino acid sequence of SEQ ID NO: 138.
55. The isolated AAV particle of any one of embodiments 15-54, wherein the AAV capsid variant further comprises one, two, or all of an amino acid other than V at position 603 (e.g., D, F, G, L, A, E, or I), an amino acid other than Q at position 604 (e.g., K, R, H, E, L, or P), and/or an amino acid other than N at position 605 (e.g., T, K, H, D, Y, S, I, or P), relative to a reference sequence numbered according to the amino acid sequence of SEQ ID NO: 5, 8, or 3636.
56. The isolated AAV particle of any one of embodiments 15-55, wherein the AAV capsid variant further comprises one, two, or all of:
   (i) the amino acid V, D, F, G, L, A, E, or I at position 596 numbered according to SEQ ID NO: 138 or at position 603 numbered according to SEQ ID NO: 5, 8, or 3636;
   (ii) the amino acid K, R, H, E, L, or P at position 597 numbered according to SEQ ID NO: 138 or at position 604 numbered according to SEQ ID NO: 5, 8, or 3636; and/or
   (iii) the amino acid N, T, K, H, D, Y, S, I, or P at position 598 numbered according to the amino acid sequence of SEQ ID NO: 138 or at position 605 numbered according to SEQ ID NO: 5, 8, or 3636.
57. The isolated AAV particle of any one of embodiments 15-56, wherein the AAV capsid variant further comprises one, two, or all of:
   (i) the amino acid V at position 596 numbered according to SEQ ID NO: 138 or at position 603 numbered according to SEQ ID NO: 5, 8, or 3636;
   (ii) the amino acid K, P, E or H at position 597 numbered according to SEQ ID NO: 138 or at position 604 numbered according to SEQ ID NO: 5, 8, or 3636; and
   (iii) the amino acid N at position 598 numbered according to the amino acid sequence of SEQ ID NO: 138 or at position 605 numbered according to SEQ ID NO: 5, 8, or 3636.
58. The isolated AAV particle of any one of embodiments 15 or 17-57, wherein the AAV capsid variant further comprises [N5], wherein [N5] comprises X13, X14, and X15, wherein:
   (a) position X13 is: V, D, F, G, L, A, E, or I;
   (b) position X14 is: Q, K, R, H, E, L, or P; and
   (c) position X15 is: N, T, K, H, D, Y, S, I, or P; and/or an amino acid modification, e.g., a conservative substitution, of any of the aforesaid amino acids in (a)-(c).
59. The isolated AAV particle of embodiment 58, wherein:
   (a) position X13 is: V, D, A, F, E, G, or L;
   (b) position X14 is: Q, K, R, L, or P; and/or
   (c) position X15 is: N, T, K, H, D, I, K, S, or P.
60. The isolated AAV particle of embodiment 58 or 59, wherein position X14 is P.
61. The isolated AAV particle of embodiment 58 or 59, wherein position X14 is K.
62. The isolated AAV particle of embodiment 58 or 59, wherein position X14 is E or H.
63. The isolated AAV particle of embodiment 58 or 59, wherein position X14 is Q.
64. The isolated AAV particle of any one of embodiment 58-63, wherein [N5] comprises VQ, AQ, DQ, FQ, VL, LQ, EQ, GQ, VP, VR, VK, QN, QS, QT, QK, QH, LN, QI, PN, QD, QP, RN, or KN.
65. The isolated AAV particle of any one of embodiments 16 or 58-64, wherein [N5] is or comprises VQN, VKN, VQT, VQK, DQN, VQH, GQN, VQI, VHN, FQN, LQN, VLN, VRN, VQS, VQY, AQN, VEN, VQD, VPN, IQN, VKK, DKN, VKT, VQP, EQN, GQT, FQK, GHN, or VPH.
66. The isolated AAV particle of any one of embodiments 16 or 58-65, wherein [N5] is or comprises VQN, AQN, VQS, DQN, VQT, VQK, VQH, FQN, VLN, LQN, VQI, EQN, GQT, VPN, VQD, VQP, VRN, or VKN.
67. The isolated AAV particle of any one of embodiments 16 or 58-66, wherein [N5] is or comprises VKN, VPN, VEN, or VHN.
68. The isolated AAV particle of any one of embodiments 16 or 58-66, wherein [N5] is or comprises VQN.
69. The isolated AAV particle of any one of embodiments 16 or 58-68, wherein [N4]-[N5] is or comprises:

(i)
(SEQ ID NO: 4851)
TGWVQN,
(SEQ ID NO: 4852)
LAAVQN,
(SEQ ID NO: 4853)
LTPVQN,
(SEQ ID NO: 4854)
SAPVQN,
(SEQ ID NO: 4855)
SSPVQN,
(SEQ ID NO: 4856)
TGRVQN,
(SEQ ID NO: 4857)
TGWAQN,
(SEQ ID NO: 4858)
TGWVQS,
(SEQ ID NO: 4859)
TLAVQN,
(SEQ ID NO: 4860)
TTSVQN,
(SEQ ID NO: 4861)
TSPVQN,
(SEQ ID NO: 4862)
TALVQN,
(SEQ ID NO: 4863)
TAWVQN,
(SEQ ID NO: 4864)
TGGVQN,
(SEQ ID NO: 4865)
TGSVQN,
(SEQ ID NO: 4866)
TGWDQN,
(SEQ ID NO: 4867)
TVSVQN,
(SEQ ID NO: 4868)
VSPVQN,
(SEQ ID NO: 4869)
VSSVQN,
(SEQ ID NO: 4870)
AAPVQN,
(SEQ ID NO: 4871)
AGPVQN,
(SEQ ID NO: 4872)
ASPVQN,
(SEQ ID NO: 4873)
ATPVQN,
(SEQ ID NO: 4874)
CSPVQN,
(SEQ ID NO: 4875)
CTPVQN,
(SEQ ID NO: 4876)
IAAVQN,
(SEQ ID NO: 4877)
IAGVQN,
(SEQ ID NO: 4878)
IASVQN,
(SEQ ID NO: 4879)
IGGVQN,
(SEQ ID NO: 4880)
IGSVQN,
(SEQ ID NO: 4881)
ILGVQN,
(SEQ ID NO: 4882)
IQPVQN,
(SEQ ID NO: 4883)
IQSVQN,
(SEQ ID NO: 4884)
ISGVQN,
(SEQ ID NO: 4885)
ISPVQN,
(SEQ ID NO: 4886)
ISSVQN,
(SEQ ID NO: 4887)
ITPVQN,
(SEQ ID NO: 4888)
LAGVQN,
(SEQ ID NO: 4889)
LAHVQN,
(SEQ ID NO: 4890)
LANVQN,
(SEQ ID NO: 4891)
LAPVQN,
(SEQ ID NO: 4892)
LAPVQT,
(SEQ ID NO: 4893)
LAQVQN,
(SEQ ID NO: 4894)
LARVQN,
(SEQ ID NO: 4895)
LASVQN,
(SEQ ID NO: 4896)
LATVQN,
(SEQ ID NO: 4897)
LGPVQN,
(SEQ ID NO: 4898)
LGQVQN,
(SEQ ID NO: 4899)
LGSVQN,
(SEQ ID NO: 4900)
LHPVQN,
(SEQ ID NO: 4901)
LKSVQN,
(SEQ ID NO: 4902)
LMAVQN,
(SEQ ID NO: 4903)
LMGVQN,
(SEQ ID NO: 4904)
LMPVQN,
(SEQ ID NO: 4905)
LMQVQN,
(SEQ ID NO: 4906)
LMSVQN,
(SEQ ID NO: 4907)
LNPVQN,
(SEQ ID NO: 4908)
LQPVQN,
(SEQ ID NO: 4909)
LQQVQN,
(SEQ ID NO: 4910)
LQRVQN,
(SEQ ID NO: 4911)
LSHVQN,
(SEQ ID NO: 4912)
LSKVQN,
(SEQ ID NO: 4913)
LSPVQK,
(SEQ ID NO: 4914)
LSPVQN,
(SEQ ID NO: 4915)
LSQVQN,
(SEQ ID NO: 4916)
LSRVQN,
(SEQ ID NO: 4917)
LSTVQN,
(SEQ ID NO: 4918)
LTAVQN,
(SEQ ID NO: 4919)
LTNVQN,
(SEQ ID NO: 4920)
LTSVQN,
(SEQ ID NO: 4921)
MAPVQN,
(SEQ ID NO: 4922)
NAQVQN,
(SEQ ID NO: 4923)
NASVQN,
(SEQ ID NO: 4924)
NMQVQN,
(SEQ ID NO: 4925)
NSPVQN,
(SEQ ID NO: 4926)
NTPVQN,
(SEQ ID NO: 4927)
NVQVQN,
(SEQ ID NO: 4928)
QAPVQN,
(SEQ ID NO: 4929)
RAAVQN,
(SEQ ID NO: 4930)
RAQVQN,
(SEQ ID NO: 4931)
RASVQN,
(SEQ ID NO: 4932)
RGGVQN,
(SEQ ID NO: 4933)
RGSVQN,
(SEQ ID NO: 4934)
RIAVQN,
(SEQ ID NO: 4935)
RIGVQN,
(SEQ ID NO: 4936)
RIPVQN,
(SEQ ID NO: 4937)
RLGVQN,
(SEQ ID NO: 4938)
RLSVQN,
(SEQ ID NO: 4939)
RMSVQN,
(SEQ ID NO: 4940)
RNSVQN,
(SEQ ID NO: 4941)
RQPVQN,
(SEQ ID NO: 4942)
RSAVQN,
(SEQ ID NO: 4943)
RSGVQN,
(SEQ ID NO: 4944)
RSPVQN,
(SEQ ID NO: 4945)
RSQVQN,
(SEQ ID NO: 4946)
RSSVQN,
(SEQ ID NO: 4947)
RSTVQN,
(SEQ ID NO: 4948)
RTAVQN,
(SEQ ID NO: 4949)
RTGVQN,
(SEQ ID NO: 4950)
RTLVQN,
(SEQ ID NO: 4951)
RTSVQN,
(SEQ ID NO: 4952)
RTTVQN,
(SEQ ID NO: 4953)
RVEVQN,
(SEQ ID NO: 4954)
SAAVQN,
(SEQ ID NO: 4955)
SAKVQN,
(SEQ ID NO: 4956)
SAMVQN,
(SEQ ID NO: 4957)
SAQVQN,
(SEQ ID NO: 4958)
SGPVQN,
(SEQ ID NO: 4959)
SMAVQN,
(SEQ ID NO: 4960)
SMGVQN,
(SEQ ID NO: 4961)
SMQVQN,
(SEQ ID NO: 4962)
SMSVQN,
(SEQ ID NO: 4963)
STPVQN,
(SEQ ID NO: 4964)
SVAVQN,
(SEQ ID NO: 4965)
SVGVQN,
(SEQ ID NO: 4966)
TAAVQN,
(SEQ ID NO: 4967)
TAGVQN,
(SEQ ID NO: 4968)
TAKVQN,
(SEQ ID NO: 4969)
TAMVQN,
(SEQ ID NO: 4970)
TANVQN,
(SEQ ID NO: 4971)
TAPVQN,
(SEQ ID NO: 4972)
TAPVQT,
(SEQ ID NO: 4973)
TAQVQN,
(SEQ ID NO: 4974)
TASVQN,
(SEQ ID NO: 4975)
TASVQT,
(SEQ ID NO: 4976)
TATVQN,
(SEQ ID NO: 4977)
TAVVQN,
(SEQ ID NO: 4978)
TAWDON,
(SEQ ID NO: 4979)
TAWVQH,
(SEQ ID NO: 4980)
TAWVQT,
(SEQ ID NO: 4981)
TGAVQN,
(SEQ ID NO: 4982)
TGCFQN,
(SEQ ID NO: 4983)
TGGAQN,
(SEQ ID NO: 4984)
TGGFQN,
(SEQ ID NO: 4985)
TGGVLN,
(SEQ ID NO: 4986)
TGGVQH,
(SEQ ID NO: 4987)
TGGVQK,
(SEQ ID NO: 4988)
TGGVQT,
(SEQ ID NO: 4989)
TGPVQN,
(SEQ ID NO: 4990)
TGSAQN,
(SEQ ID NO: 4991)
TGSLON,
(SEQ ID NO: 4992)
TGSVQH,
(SEQ ID NO: 4993)
TGSVQI,
(SEQ ID NO: 4994)
TGSVQS,
(SEQ ID NO: 4995)
TGSVQT,
(SEQ ID NO: 4996)
TGTVQN,
(SEQ ID NO: 4997)
TGWEQN,
(SEQ ID NO: 4998)
TGWFQN,
(SEQ ID NO: 4999)
TGWGQT,
(SEQ ID NO: 5000)
TGWVPN,
(SEQ ID NO: 5001)
TGWVQD,
(SEQ ID NO: 5002)
TGWVQP,
(SEQ ID NO: 5003)
TGWVQT,
(SEQ ID NO: 5004)
TGWVRN,
(SEQ ID NO: 5005)
TKAVQN,
(SEQ ID NO: 5006)
TKPVQN,
(SEQ ID NO: 5007)
TKQVQN,
(SEQ ID NO: 5008)
TKSVQN,
(SEQ ID NO: 5009)
TLPVQN,
(SEQ ID NO: 5010)
TLQVQN,
(SEQ ID NO: 5011)
TMAVQN,
(SEQ ID NO: 5012)
TMGVQN,
(SEQ ID NO: 5013)
TMKVQN,
(SEQ ID NO: 5014)
TMNVQN,
(SEQ ID NO: 5015)
TMPVQN,
(SEQ ID NO: 5016)
TMQVQN,
(SEQ ID NO: 5017)
TMSVKN,
(SEQ ID NO: 5018)
TMSVQN,
(SEQ ID NO: 5019)
TMSVQT,
(SEQ ID NO: 5020)
TMTVQN,
(SEQ ID NO: 5021)
TNAVQN,
(SEQ ID NO: 5022)
TNQVQN,
(SEQ ID NO: 5023)
TNSVQN,
(SEQ ID NO: 5024)
TPPVQN,
(SEQ ID NO: 5025)
TQHVQN,
(SEQ ID NO: 5026)
TQKVQN,
(SEQ ID NO: 5027)
TQMVQN,
(SEQ ID NO: 5028)
TQNVQN,
(SEQ ID NO: 5029)
TQPVQN,
(SEQ ID NO: 5030)
TQQVQN,
(SEQ ID NO: 5031)
TQTVQN,
(SEQ ID NO: 5032)
TRWDQN,
(SEQ ID NO: 5033)
TSAVQN,
(SEQ ID NO: 5034)
TSGVQN,
(SEQ ID NO: 5035)
TSHVQN,
(SEQ ID NO: 5036)
TSKVQN,
(SEQ ID NO: 5037)
TSLVQN,
(SEQ ID NO: 5038)
TSMVQN,
(SEQ ID NO: 5039)
TSPDQN,
(SEQ ID NO: 5040)
TSQVQN,
(SEQ ID NO: 5041)
TSSVQN,
(SEQ ID NO: 5042)
TSSVQT,
(SEQ ID NO: 5043)
TSTVQN,
(SEQ ID NO: 5044)
TSVVQN,
(SEQ ID NO: 5045)
TTAVQN,
(SEQ ID NO: 5046)
TTGVQN,
(SEQ ID NO: 5047)
TTKVQN,
(SEQ ID NO: 5048)
TTPVQN,
(SEQ ID NO: 5049)
TTPVQT,
(SEQ ID NO: 5050)
TTQVQN,
(SEQ ID NO: 5051)
TTTVQN,
(SEQ ID NO: 5052)
TVAVQN,
(SEQ ID NO: 5053)
TVAVQT,
(SEQ ID NO: 5054)
TVGVQN,
(SEQ ID NO: 5055)
TVQVQN,
(SEQ ID NO: 5056)
TVSVKN,
(SEQ ID NO: 5057)
TVWVQK,
(SEQ ID NO: 5058)
VAAVQN,
(SEQ ID NO: 5059)
VAGVQN,
(SEQ ID NO: 5060)
VAKVQN,
(SEQ ID NO: 5061)
VANVQN,
(SEQ ID NO: 5062)
VAQVQN,
(SEQ ID NO: 5063)
VASVQN,
(SEQ ID NO: 5064)
VATVQN,
(SEQ ID NO: 5065)
VGGVQN,
(SEQ ID NO: 5066)
VGKVQN,
(SEQ ID NO: 5067)
VGNVQN,
(SEQ ID NO: 5068)
VGSVQN,
(SEQ ID NO: 5069)
VHPVQN,
(SEQ ID NO: 5070)
VKAVQN,
(SEQ ID NO: 5071)
VKPVQN,
(SEQ ID NO: 5072)
VKQVQN,
(SEQ ID NO: 5073)
VLPVQN,
(SEQ ID NO: 5074)
VLSVQN,
(SEQ ID NO: 5075)
VMAVQN,
(SEQ ID NO: 5076)
VMQVQN,
(SEQ ID NO: 5077)
VMSVQN,
(SEQ ID NO: 5078)
VNAVQN,
(SEQ ID NO: 5079)
VNGVQN,
(SEQ ID NO: 5080)
VNSVQN,
(SEQ ID NO: 5081)
VQAVQN,
(SEQ ID NO: 5082)
VQNVQN,
(SEQ ID NO: 5083)
VQPVQN,
(SEQ ID NO: 5084)
VQQVQN,
(SEQ ID NO: 5085)
VQSVQN,
(SEQ ID NO: 5086)
VQTVQN,
(SEQ ID NO: 5087)
VRPVQN,
(SEQ ID NO: 5088)
VSAVQN,
(SEQ ID NO: 5089)
VSGVQN,
(SEQ ID NO: 5090)
VSNVQN,
(SEQ ID NO: 5091)
VSPVQT,
(SEQ ID NO: 5092)
VSQVQN,
(SEQ ID NO: 5093)
VSRVQN,
(SEQ ID NO: 5094)
VSSVQK,
(SEQ ID NO: 5095)
VSSVQT,
(SEQ ID NO: 5096)
VSTVQN,
(SEQ ID NO: 5097)
VTAVQN,
(SEQ ID NO: 5098)
VTGVQN,
(SEQ ID NO: 5099)
VTKVQN,
(SEQ ID NO: 5100)
VTPVQN,
(SEQ ID NO: 5101)
VTSVQN,
(SEQ ID NO: 5102)
TGLVQN,
(SEQ ID NO: 5103)
TGWVKN,
(SEQ ID NO: 5104)
PGWVQN,
(SEQ ID NO: 5105)
TGWVQH,
(SEQ ID NO: 5106)
LSGVQN,
(SEQ ID NO: 5107)
LSSVQN,or
(SEQ ID NO: 5108)
LVPVQN;

(ii) an amino acid sequence comprising any portion of an amino acid sequence in (i), e.g., any 2, 3, 4, or 5 amino acids, e.g., consecutive amino acids, thereof;
(iii) an amino acid sequence comprising at least one, two, or three but no more than four modifications, e.g., substitutions (e.g., conservative substitutions), insertions, or deletions, relative to any of the amino acid sequences in (i); or
(iv) an amino acid sequence comprising at least one, two, or three but no more than four different amino acids, relative to any one of the amino acid sequences in (i).
70. The isolated AAV particle of any one of embodiments 16 or 58-69, wherein [N4]-[N5] is or comprises TGWVQN (SEQ ID NO: 4851), LSPVKN (SEQ ID NO: 5109), or TGWVPN (SEQ ID NO: 5000).
71. The isolated AAV particle of embodiment 16-70, wherein:
   (i) [N1] is or comprises: PLNGA (SEQ ID NO: 3679), QLNGA (SEQ ID NO: 4685), PLDGA (SEQ ID NO: 4691), PLDSS (SEQ ID NO: 4705), ALNGA (SEQ ID NO: 4686), PLNGS (SEQ ID NO: 4687), PLNGG (SEQ ID NO: 4689), SLNGA (SEQ ID NO: 4684), PLNGN (SEQ ID NO: 4693), PLNGT (SEQ ID NO: 4690), ALDGA (SEQ ID NO: 4698), PLDSA (SEQ ID NO: 4701), SLDGA (SEQ ID NO: 4694), TLNGA (SEQ ID NO: 4708), or PINGA (SEQ ID NO: 4697);
   (ii) [N2] is or comprises: VHLY (SEQ ID NO: 4680), VHHY (SEQ ID NO: 4683), VHVY (SEQ ID NO: 4682), or VHIY (SEQ ID NO: 4681);
   (iii) [N3] is or comprises: AQAQ (SEQ ID NO: 4737), AQPQ (SEQ ID NO: 4739), AKAQ (SEQ ID NO: 4741), DQAQ (SEQ ID NO: 4744), or SQAQ (SEQ ID NO: 4738);
   (iv) [N4] is or comprises: LSP, TGW, TMS, TTK, TGS, TTS, TSP, TMK, VAQ, TGG, TAW, VKQ, SAP, LSK, LAP, LAQ, VAS, TAK, SAK, TGC, TQK, TGR, TVA, SSP, TTQ, TAQ, RIA, RAS, TTP, LAS, LTP, STP, VSQ, TMQ, TSK, VSP, TVQ, VTA, RQP, ISG, VRP, LGP, TNQ, VQQ, VAN, AAP, RST, TMA, IQP, IAS, TVS, RGS, NSP, LQP, VTG, VMQ, SMA, VGK, IQS, CSP, LQR, TPP, VTK, AGP, LAR, TTT, TLQ, VAK, RAA, TVG, LNP, LSQ, TKP, TNA, LAT, VTP, VQA, CTP, TAG, TSQ, TMN, TST, VKP, ASP, VAA, LKS, IAA, TAA, TKA, VSN, TAP, LMP, LHP, RAQ, LTN, RTT, TSV, TLA, RMS, VGN, LMQ, TAT, VHP, ISS, TRW, TMT, RSS, PGW, RTG, VAT, VTS, VSS, TSS, TNS, VKA, SGP, TGP, TAM, TQP, TQQ, VSR, VLP, LGS, VSA, VLS, TQH, QAP, NAQ, ATP, VQP, TTA, LAA, RSG, LMA, TMP, LAN, VST, SAQ, NTP, TGL, TLP, TAV, RLG, RTL, TQM, ITP, TVW, RSA, TAS, TMG, VQS, ISP, VGG, TAL, LAG, RTA, RSP, LAH, TSL, RLS, LMG, SMQ, TQT, VGS, VSG, VMA, IGG, IAG, LSH, VQT, RNS, TKQ, LGQ, NMQ, NVQ, RGG, VMS, TTG, LSR, MAP, ILG, TGT, TSH, RIG, SAM, TSM, SMG, SMS, TSG, TGA, VNS, VAG, IGS, VNG, LSS, LTA, VQN, TKS, SVG, NAS, TSA, TAN, LTS, RSQ, RIP, LVP, RVE, SVA, LSG, LQQ, LST, SAA, RTS, TQN, VNA, or LMS; and/or
   (v) [N5] is or comprises: VQN, DQN, VQH, FQN, VQD, VQS, VQT, VRN, AQN, VQP, VPN, VKN, VQK, EQN, VQI, LQN, GQT, or VLN.
72. The isolated AAV particle of embodiment 16, wherein:
   (i) [N1] is or comprises: SLNGA (SEQ ID NO: 4684), QLNGA (SEQ ID NO: 4685), ALNGA (SEQ ID NO: 4686), PLNGS (SEQ ID NO: 4687), PVNGA (SEQ ID NO: 4688), PLNGA (SEQ ID NO: 3679), PLNGG (SEQ ID NO: 4689), PLNGT (SEQ ID NO: 4690), PLDGA (SEQ ID NO: 4691), QLNGS (SEQ ID NO: 4692), PLNGN (SEQ ID NO: 4693), SLDGA (SEQ ID NO: 4694), HLNGA (SEQ ID NO: 4695), ALNGT (SEQ ID NO: 4696), PINGA (SEQ ID NO: 4697), ALDGA (SEQ ID NO: 4698), PLNCA (SEQ ID NO: 4699), PLNGQ (SEQ ID NO: 4700), PLDSA (SEQ ID NO: 4701), RLDGA (SEQ ID NO: 4702), QLNGN (SEQ ID NO: 4703), PLNGY (SEQ ID NO: 4704), or PLDSS (SEQ ID NO: 4705);
   (ii) [N2] is or comprises: VHLY (SEQ ID NO: 4680) or VHVY (SEQ ID NO: 4682);
   (iii) [N3] is or comprises: AQAQ (SEQ ID NO: 4737), SQAQ (SEQ ID NO: 4738), AQPQ (SEQ ID NO: 4739), or AQSQ (SEQ ID NO: 4740);
   (iv) [N4] is or comprises: TGW, TGL, TGS, TGG, TAW, TGR, TAS, LSS, TSS, SSL, SSS, TLS, TVS, VSS, TSP, VSP, TMS, LSP, VAS, TAL, TTS, TLP, VLP, RGW, LSG, LAS, SSP, LLP, STS, TSA, TTP, SAL, LGS, VTP, VSA, IGW, TGF, LTP, TLA, LSA, TVG, TAP, TMP, TSL, VQS, SSM, SLP, VSQ, RSS, TST, VMS, TTA, TQP, LST, LAP, TVA, RLS, TGY, TSG, TAG, VMP, TSQ, TMA, VGS, TSW, TGV, TGT, TLG, LMP, VQP, TGM, SMS, SQL, IGS, RSV, TAA, STP, LSQ, TAQ, TGP, ASP, VSG, SAP, TLQ, LQP, TAT, TGQ, ATS, IGG, VAA, TSM, TVW, TAM, TGA, VAT, QSP, TQA, VQA, RSP, LAT, VAQ, LAA, RST, RTL, LGT, LMS, LGP, RTS, SQP, VLG, SVS, TMQ, SAV, LAG, SGP, TNS, RLT, TTQ, SAA, TSV, RLG, RAS, STQ, CSP, SAG, ALP, VTS, ISP, SVG, LTS, TTT, RSG, TQL, LNP, TVQ, IAS, LAQ, LSR, LSN, TTG, TSN, SMA, TKS, SVA, TQQ, VQQ, RLP, SAM, TAV, TQW, SSR, TQT, VNS, RSA, LMG, RQS, LVG, VTA, RTT, SMG, VMA, TKP, SAQ, NSP, ATP, VAG, RGS, VKP, RMS, NLP, NAL, RTP, RQL, VQG, VTG, VST, NAS, RVE, ATG, AMS, RNS, VMQ, SMQ, LQQ, TMG, LGQ, TSH, AAP, RSQ, TYS, ITP, VAK, TQM, TKA, SQQ, ISG, VSR, RTA, RML, SQM, VAN, CTP, ISS, AGP, TAK, RTG, LHP, TMT, AQP, QAP, RQP, LKS, NTT, TSK, RYS, KSS, NTP, VGG, IAA, LMA, MAP, VHP, VLS, LAN, ATQ, TNA, TAN, VSN, AAA, AVG, LTA, SAN, RAG, RQG, TLR, LSH, SAF, RAA, IQP, ILG, VNG, SVQ, LSK, TNG, RTQ, TMN, RGG, TTR, VRP, VKA, LAR, NQP, TMK, TYA, TQK, TTK, IAG, TQN, LAH, NTQ, RQQ, RAQ, TKQ, TQH, TNQ, LMQ, VNA, VQT, TQR, VGK, VKQ, IQS, LQR, TMM, VGN, RIG, SAK, RIA, VQN, NVQ, RIP, NAQ, NMQ, TPS, LTN, VTK, PGW, LPP, SPP, TPA, or TGC; and/or
   (v) [N5] is or comprises: VQN, VKN, VQT, VQK, DQN, VQH, GQN, VQI, VHN, FQN, LQN, VLN, VRN, VQS, VQY, AQN, VEN, VQD.
73. The isolated AAV particle of any one of embodiments 15, 16, 27-30, 42-47, or 54-58, which comprises:
   (i) the amino acid sequence of any of SEQ ID NOs: 139-1138;
   (ii) an amino acid sequence comprising any portion of an amino acid sequence in (i), e.g., any 2, 3, 4, 5, 6, 7, 8, 9, 10, 11, 12, 13, 14, 15, 16, 17, or 18 amino acids, e.g., consecutive amino acids, thereof;
   (iii) an amino acid sequence comprising at least one, two, or three but no more than four modifications, e.g., substitutions (e.g., conservative substitutions), insertions, or deletions, relative to any of the amino acid sequences in (i); or
   (iv) an amino acid sequence comprising at least one, two, or three but no more than four different amino acids, relative to any one of the amino acid sequences in (i).
74. The isolated AAV particle of any one of embodiments 15-71 or 73, which comprises:
   (i) the amino acid sequence of any of SEQ ID NOs: 139-476;
   (ii) an amino acid sequence comprising any portion of an amino acid sequence in (i), e.g., any 2, 3, 4, 5, 6, 7, 8, 9, 10, 11, 12, 13, 14, 15, 16, 17, or 18 amino acids, e.g., consecutive amino acids, thereof;
   (iii) an amino acid sequence comprising at least one, two, or three but no more than four modifications, e.g., substitutions (e.g., conservative substitutions), insertions, or deletions, relative to any of the amino acid sequences in (i); or
   (iv) an amino acid sequence comprising at least one, two, or three but no more than four different amino acids, relative to any one of the amino acid sequences in (i).
75. An isolated, e.g., recombinant, AAV particle comprising an AAV capsid variant and a nucleic acid comprising a transgene encoding an antibody molecule which binds to tau (e.g., human tau), wherein the AAV capsid variant comprises one, two, three, four, or all of:
   (i) an [N1], wherein [N1] is or comprises: PLNGA (SEQ ID NO: 3679), SLNGA (SEQ ID NO: 4684), QLNGA (SEQ ID NO: 4685), ALNGA (SEQ ID NO: 4686), PLNGS (SEQ ID NO: 4687), PVNGA (SEQ ID NO: 4688), PLNGG (SEQ ID NO: 4689), PLNGT (SEQ ID NO: 4690), PLDGA (SEQ ID NO: 4691), QLNGS (SEQ ID NO: 4692), PLNGN (SEQ ID NO: 4693), SLDGA (SEQ ID NO: 4694), HLNGA (SEQ ID NO: 4695), ALNGT (SEQ ID NO: 4696), PINGA (SEQ ID NO: 4697), ALDGA (SEQ ID NO: 4698), PLNCA (SEQ ID NO: 4699), PLNGQ (SEQ ID NO: 4700), PLDSA (SEQ ID NO: 4701), RLDGA (SEQ ID NO: 4702), QLNGN (SEQ ID NO: 4703), PLNGY (SEQ ID NO: 4704), or PLDSS (SEQ ID NO: 4705);
   (ii) an [N2] wherein [N2] is or comprises: VHLY (SEQ ID NO: 4680) or VHVY (SEQ ID NO: 4682);
   (iii) an [N3] wherein [N3] is or comprises: AQAQ (SEQ ID NO: 4737), SQAQ (SEQ ID NO: 4738), AQPQ (SEQ ID NO: 4739), or AQSQ (SEQ ID NO: 4740);
   (iv) an [N4] wherein [N4] is or comprises: TGW, TGL, TGS, TGG, TAW, TGR, TAS, LSS, TSS, SSL, SSS, TLS, TVS, VSS, TSP, VSP, TMS, LSP, VAS, TAL, TTS, TLP, VLP, RGW, LSG, LAS, SSP, LLP, STS, TSA, TTP, SAL, LGS, VTP, VSA, IGW, TGF, LTP, TLA, LSA, TVG, TAP, TMP, TSL, VQS, SSM, SLP, VSQ, RSS, TST, VMS, TTA, TQP, LST, LAP, TVA, RLS, TGY, TSG, TAG, VMP, TSQ, TMA, VGS, TSW, TGV, TGT, TLG, LMP, VQP, TGM, SMS, SQL, IGS, RSV, TAA, STP, LSQ, TAQ, TGP, ASP, VSG, SAP, TLQ, LQP, TAT, TGQ, ATS, IGG, VAA, TSM, TVW, TAM, TGA, VAT, QSP, TQA, VQA, RSP, LAT, VAQ, LAA, RST, RTL, LGT, LMS, LGP, RTS, SQP, VLG, SVS, TMQ, SAV, LAG, SGP, TNS, RLT, TTQ, SAA, TSV, RLG, RAS, STQ, CSP, SAG, ALP, VTS, ISP, SVG, LTS, TTT, RSG, TQL, LNP, TVQ, IAS, LAQ, LSR, LSN, TTG, TSN, SMA, TKS, SVA, TQQ, VQQ, RLP, SAM, TAV, TQW, SSR, TQT, VNS, RSA, LMG, RQS, LVG, VTA, RTT, SMG, VMA, TKP, SAQ, NSP, ATP, VAG, RGS, VKP, RMS, NLP, NAL, RTP, RQL, VQG, VTG, VST, NAS, RVE, ATG, AMS, RNS, VMQ, SMQ, LQQ, TMG, LGQ, TSH, AAP, RSQ, TYS, ITP, VAK, TQM, TKA, SQQ, ISG, VSR, RTA, RML, SQM, VAN, CTP, ISS, AGP, TAK, RTG, LHP, TMT, AQP, QAP, RQP, LKS, NTT, TSK, RYS, KSS, NTP, VGG, IAA, LMA, MAP, VHP, VLS, LAN, ATQ, TNA, TAN, VSN, AAA, AVG, LTA, SAN, RAG, RQG, TLR, LSH, SAF, RAA, IQP, ILG, VNG, SVQ, LSK, TNG, RTQ, TMN, RGG, TTR, VRP, VKA, LAR, NQP, TMK, TYA, TQK, TTK, IAG, TQN, LAH, NTQ, RQQ, RAQ, TKQ, TQH, TNQ, LMQ, VNA, VQT, TQR, VGK, VKQ, IQS, LQR, TMM, VGN, RIG, SAK, RIA, VQN, NVQ, RIP, NAQ, NMQ, TPS, LTN, VTK, PGW, LPP, SPP, TPA, or TGC; and/or
   (v) an [N5] wherein [N5] is or comprises: VQN, VKN, VQT, VQK, DQN, VQH, GQN, VQI, VHN, FQN, LQN, VLN, VRN, VQS, VQY, AQN, VEN, VQD; and/or wherein the AAV capsid variant comprises an amino acid modification, e.g., a conservative substitution, of any of the aforesaid amino acids in (i)-(v).
76. The isolated AAV particle of embodiment 72 or 75, wherein the AAV capsid variant comprises:
   (i) the amino acid sequence of any of SEQ ID NOs: 140, 142-144, 148-150, 154-158, 160, 161, 163, 165, 166, 168, 170, 171, 173-175, 177-179, 181, 182, 184-197, 199-214, 218-222, 224, 225, 227-241, 243-253, 255-262, 265, 267, 268, 270, 271, 273, 274, 276, 277, 279, 282, 284-286, 288-296, 300-310, 312, 315, 317, 318, 320-323, 326, 327, 331, 332, 334, 336, 337, 339, 340, 341, 343, 344, 346, 349, 351, 352, 356-363, 365-367, 369, 370, 372-376, 378-381, 383-389, 392, 393, 395, 397-400, 404, 407, 408, 411, 412, 415, 417, 420-430, 432, 433, 435-438, 441, 442, 446-448, 451-453, 456, 458, 460, 461, 465, 467-469, 471-473, 475, 476, 478, 480, 482, 485, 488, 490, 492, 493, 495, 498, 500-503, 505, 507, 509, 510, 517, 522-526, 528, 535-538, 540, 543-545, 547, 551, 552, 557, 559, 561, 564, 568, 570, 572-574, 577, 585-588, 592-594, 596, 601, 602, 605, 612, 616, 619, 622, 624, 627, 628, 635, 640, 641, 646, 658, 660, 665, 666, 675, 678, 680, 683, 684, 689, 693, 695, 707, 711, 718, 719, 724, 727, 735, 740, 748, 751, 755, 758, 759, 765, 766, 768, 778, 783, 787, 791, 797, 801, 804, 817, 821, 832, 841, 852, 856, 861, 862, 864, 894, 906, 911, 913, 924, 929, 945, 959, 961, 970, 975, 980, 983, 988, 992, 1009, 1015, 1019, 1027, 1032, 1036, 1038, 1047, 1051, 1061, 1077, 1081, 1095, or 1113;
   (ii) an amino acid sequence comprising any portion of an amino acid sequence in (i), e.g., any 2, 3, 4, 5, 6, 7, 8, 9, 10, 11, 12, 13, 14, 15, 16, 17, or 18 amino acids, e.g., consecutive amino acids, thereof;
   (iii) an amino acid sequence comprising at least one, two, or three but no more than four modifications, e.g., substitutions (e.g., conservative substitutions), insertions, or deletions, relative to any of the amino acid sequences in (i); or
   (iv) an amino acid sequence comprising at least one, two, or three but no more than four different amino acids, relative to any one of the amino acid sequences in (i).
77. The isolated AAV particle of any one of embodiments 16 or 58-75, wherein [N1]-[N2]-[N3]-[N4]-[N5] is or comprises the amino acid sequence of PLNGAVHLYAQAQTGWVPN (SEQ ID NO: 314).
78. The isolated AAV particle of any one of embodiments 16 or 58-75, wherein [N1]-[N2]-[N3]-[N4]-[N5] is or comprises the amino acid sequence of PLNGAVHLYAQAQLSPVKN (SEQ ID NO: 566).
79. The isolated AAV particle of any one of embodiments 16 or 58-76, wherein [N1]-[N2]-[N3]-[N4]-[N5] is or comprises the amino acid sequence of PLNGAVHLYAQAQTGWVQN (SEQ ID NO: 476).
80. The isolated AAV particle of any one of embodiments 16 or 58-75, wherein [N1]-[N2]-[N3]-[N4]-[N5] is or comprises the amino acid sequence of:
   (i) the amino acid sequence of any of SEQ ID NOs: 1200-1203, 40-136, 314, 325, 491, 499, 529, 558, 566, 576, 603, 610, 625, 631, 648, 649, 700, 703, 720, 755, 763, 765, 771, 791, 804, 816, 818, 819, 828, 859, 864, 871, 885, 946, 960, 966, 978, 979, 1016, 1033, 1032, 1037, 1058, 1081, 1100, 1122, or 1204-1223;
   (ii) an amino acid sequence comprising any portion of an amino acid sequence in (i), e.g., any 2, 3, 4, 5, 6, 7, 8, 9, 10, 11, 12, 13, 14, 15, 16, 17, or 18 amino acids, e.g., consecutive amino acids, thereof;
   (iii) an amino acid sequence comprising at least one, two, or three but no more than four modifications, e.g., substitutions (e.g., conservative substitutions), insertions, or deletions, relative to any of the amino acid sequences in (i); or
   (iv) an amino acid sequence comprising at least one, two, or three but no more than four different amino acids, relative to any one of the amino acid sequences in (i).
81. The isolated AAV particle of any one of embodiments 15-80, wherein [N1]-[N2] is present in loop VIII.
82. The isolated AAV particle of any one of embodiments 16 or 58-81, wherein [N3], [N4], and/or [N5] is present in loop VIII.
83. The isolated AAV particle of any one of embodiments 15-82, wherein [N1]-[N2]-[N3]-[N4]-[N5] is present in loop VIII.
84. The isolated AAV particle of any one of embodiments 15-83, wherein the AAV capsid variant comprises an amino acid other than A at position 587 and/or an amino acid other than Q at position 588, numbered according to SEQ ID NO: 138.
85. The isolated AAV particle of any one of embodiments 15-84, which comprises:
   (i) the amino acid P, Q, A, H, K, L, R, S, or T (e.g., P, Q, A, S, or T) at position 587, numbered according to SEQ ID NO: 138 or 3636; and/or
   (ii) the amino acid L, I, V, H, or R (e.g., L or I) at position 588, numbered according to SEQ ID NO: 5, 8, 138 or 3636.
86. The isolated AAV particle of any one of embodiments 15-85, wherein [N1] is present immediately subsequent to position 586, relative to a reference sequence numbered according to the amino acid sequence of SEQ ID NO: 5, 8, 138, or 3636.
87. The isolated AAV particle of any one of embodiments 15-86, wherein [N1] is present immediately subsequent to position 586, and replaces positions 587 and 588 (e.g., A587 and Q588), numbered according to the amino acid sequence of SEQ ID NO: 138.
88. The isolated AAV particle of any one of embodiments 15-87, wherein [N1] replaces positions 587 and 588 (e.g., A587 and Q588), numbered according to the amino acid sequence of SEQ ID NO: 138.
89. The isolated AAV particle of any one of embodiments 15-88, wherein [N1] corresponds to positions 587-591 of SEQ ID NO: 5, 8, or 3636.
90. The isolated AAV particle of any one of embodiments 16 or 58-89, wherein [N1]-[N2]-[N3]-[N4]-[N5] is present immediately subsequent to position 586, numbered according to the amino acid sequence of SEQ ID NO: 5, 8, 138, or 3636.
91. The isolated AAV particle of any one of embodiments 15-90, wherein [N2] is present immediately subsequent to [N1].
92. The isolated AAV particle of any one of embodiments 15-91, wherein [N2] is present immediately subsequent to [N1], wherein [N1] is present immediately subsequent to position 586, relative to a reference sequence numbered according to the amino acid sequence of SEQ ID NO: 5, 8, 138, or 3636.
93. The isolated AAV particle of any one of embodiments 15-92, wherein [N2] is present immediately subsequent to [N1], wherein [N1] is present immediately subsequent to position 586, relative to a reference sequence numbered according to the amino acid sequence of SEQ ID NO: 5, 8, 138, or 3636.
94. The isolated AAV particle of any one of embodiments 15-93, wherein [N2] is present immediately subsequent to [N1], wherein [N1] is present immediately subsequent to position 586 and replaces positions 587 and 588 (e.g., A587 and Q588), numbered according to SEQ ID NO: 138.
95. The isolated AAV particle of any one of embodiments 15-94, wherein [N2] corresponds to positions 592-595 of SEQ ID NO: 5, 8, or 3636.
96. The isolated AAV particle of any one of embodiments 15-95, wherein [N1]-[N2] replaces positions 587 and 588 (e.g., A587 and Q588), numbered according to SEQ ID NO: 138.
97. The isolated AAV particle of any one of embodiments 15-96, wherein [N1]-[N2] is present immediately subsequent to position 586, numbered according to SEQ ID NO: 138.
98. The isolated AAV particle of any one of embodiments 15-97, wherein [N1]-[N2] is present immediately subsequent to position 586 and replaces positions 587 and 588 (e.g., A587 and Q588), numbered according to SEQ ID NO: 138.
99. The isolated AAV particle of any one of embodiments 15-98, wherein [N1]-[N2] corresponds to positions 587-595 of SEQ ID NO: 5, 8, or 3636.
100. The isolated AAV particle of any one of embodiments 16 or 35-99, wherein [N3] is present immediately subsequent to position 588, relative to a reference sequence numbered according to the amino acid sequence of SEQ ID NO: 138.
101. The isolated AAV particle of any one of embodiments 16 or 35-100, wherein [N3] replaces positions 589-592 (e.g., A589, Q590, A591, Q592), numbered according to the amino acid sequence of SEQ ID NO: 138.
102. The isolated AAV particle of any one of embodiments 16 or 35-101, wherein [N3] is present immediately subsequent to position 588, and replaces positions 589-592 (e.g., A589, Q590, A591, Q592), numbered according to the amino acid sequence of SEQ ID NO: 138.
103. The isolated AAV particle of any one of embodiments 16 or 35-102, wherein [N3] corresponds to positions 596-599 of SEQ ID NO: 5, 8, or 3636.
104. The isolated AAV particle of any one of embodiments 16 or 35-103, wherein [N1]-[N2]-[N3] is present immediately subsequent to position 586, numbered according to SEQ ID NO: 138.
105. The isolated AAV particle of any one of embodiments 16 or 35-104, wherein [N1]-[N2]-[N3] replaces positions 587-592 (e.g., A587, Q588, A589, Q590, A591, Q592), numbered according to SEQ ID NO: 138.
106. The isolated AAV particle of any one of embodiments 16 or 35-105, wherein [N1]-[N2]-[N3] is present immediately subsequent to position 586 and replaces positions 587-592 (e.g., A587, Q588, A589, Q590, A591, Q592), numbered according to SEQ ID NO: 138.
107. The isolated AAV particle of any one of embodiments 16 or 35-106, wherein [N1]-[N2]-[N3] corresponds to positions 587-599 of SEQ ID NO: 5, 8, or 3636.
108. The isolated AAV particle of any one of embodiments 16 or 47-107, wherein [N4] is present immediately subsequent to position 592, relative to a reference sequence numbered according to the amino acid sequence of SEQ ID NO: 138.
109. The isolated AAV particle of any one of embodiments 16 or 47-108, wherein [N4] replaces positions 593-595 (e.g., T593, G594, W595), numbered according to the amino acid sequence of SEQ ID NO: 138.
110. The isolated AAV particle of any one of embodiments 16 or 47-109, wherein [N4] is present immediately subsequent to position 592, and replaces positions 593-595 (e.g., T593, G594, W595), numbered according to the amino acid sequence of SEQ ID NO: 138.
111. The isolated AAV particle of any one of embodiments 16 or 47-110, wherein [N4] corresponds to positions 600-602 of SEQ ID NO: 5, 8, or 3636.
112. The isolated AAV particle of any one of embodiments 16 or 47-111, wherein [N3]-[N4] is present immediately subsequent to position 588, numbered according to SEQ ID NO: 138.
113. The isolated AAV particle of any one of embodiments 16 or 47-112, wherein [N3]-[N4] replaces positions 589-595 (e.g., A589, Q590, A591, Q592, T593, G594, W595), numbered according to the amino acid sequence of SEQ ID NO: 138.
114. The isolated AAV particle of any one of embodiments 16 or 47-113, wherein [N3]-[N4] is present immediately subsequent to 588, and replaces positions 589-595 (e.g., A589, Q590, A591, Q592, T593, G594, W595), numbered according to the amino acid sequence of SEQ ID NO: 138.
115. The isolated AAV particle of any one of embodiments 16 or 47-114, wherein [N3]-[N4] corresponds to positions 596-602 of SEQ ID NO: 5, 8, or 3636.
116. The isolated AAV particle of any one of embodiments 16 or 47-115, wherein [N1]-[N2]-[N3]-[N4] is present immediately subsequent to position 586, numbered according to SEQ ID NO: 138.
117. The isolated AAV particle of any one of embodiments 16 or 47-116, wherein [N1]-[N2]-[N3]-[N4] replaces positions 587-595 (e.g., A587, Q588, A589, Q590, A591, Q592, T593, G594, W595), numbered according to SEQ ID NO: 138.
118. The isolated AAV particle of any one of embodiments 16 or 47-117, wherein [N1]-[N2]-[N3]-[N4] is present immediately subsequent to position 586 and replaces positions 587-595 (e.g., A587, Q588, A589, Q590, A591, Q592, T593, G594, W595), numbered according to SEQ ID NO: 138.
119. The isolated AAV particle of any one of embodiments 16 or 47-118, wherein [N1]-[N2]-[N3]-[N4] corresponds to positions 587-602 of SEQ ID NO: 5, 8, or 3636.
120. The isolated AAV particle of any one of embodiments 16 or 58-119, wherein [N5] is present immediately subsequent to position 595, relative to a reference sequence numbered according to the amino acid sequence of SEQ ID NO: 138.
121. The isolated AAV particle of any one of embodiments 16 or 58-120, wherein [N5] replaces positions 596-598 (e.g., V596, Q597, N598), numbered according to the amino acid sequence of SEQ ID NO: 138.
122. The isolated AAV particle of any one of embodiments 16 or 63-121, wherein [N5] is present immediately subsequent to position 595, and replaces positions 596-598 (e.g., V596, Q597, N598), numbered according to the amino acid sequence of SEQ ID NO: 138.
123. The isolated AAV particle of any one of embodiments 16 or 63-122, wherein [N5] corresponds to positions 603-605 of SEQ ID NO: 5, 8, or 3636.
124. The isolated AAV particle of any one of embodiments 16 or 63-123, wherein [N4]-[N5] corresponds to positions 600-605 of SEQ ID NO: 5, 8, or 3636.
125. The isolated AAV particle of any one of embodiments 16 or 63-124, wherein [N1]-[N2]-[N3]-[N4]-[N5] replaces positions 587-598 (e.g., A587, Q588, A589, Q590, A591, Q592, T593, G594, W595, V596, Q597, N598), numbered according to SEQ ID NO: 138.
126. The isolated AAV particle of any one of embodiments 16 or 63-125, wherein [N1]-[N2]-[N3]-[N4]-[N5] is present immediately subsequent to position 586 and replaces positions 587-598 (e.g., A587, Q588, A589, Q590, A591, Q592, T593, G594, W595, V596, Q597, N598), numbered according to SEQ ID NO: 138.
127. The isolated AAV particle of any one of embodiments 16 or 63-126, wherein [N1]-[N2]-[N3]-[N4]-[N5] corresponds to positions 587-605 of SEQ ID NO: 5, 8, or 3636.
128. The isolated AAV particle of any one of embodiments 15-127, wherein the AAV capsid variant comprises from N-terminus to C-terminus, [N1]-[N2].
129. The isolated AAV particle of any one of embodiments 16 or 30-128, wherein the AAV capsid variant comprises from N-terminus to C-terminus, [N1]-[N2]-[N3].
130. The isolated AAV particle of any one of embodiments 16 or 47-129, wherein the AAV capsid variant comprises from N-terminus to C-terminus, [N1]-[N2]-[N3]-[N4].
131. The isolated AAV particle of any one of embodiments 16 or 58-130, wherein the AAV capsid variant comprises from N-terminus to C-terminus, [N1]-[N2]-[N3]-[N4]-[N5].
132. An isolated, e.g., recombinant, AAV particle comprising an AAV capsid variant and a nucleic acid comprising a transgene encoding an antibody molecule which binds to tau (e.g., human tau), wherein the AAV capsid variant comprises [A][B], wherein [A] comprises the amino acid sequence of PLNGA (SEQ ID NO: 3679), and [B] comprises X1, X2, X3, X4, wherein:
   (i) X1 is: V, I, L, A, F, D, or G;
   (ii) X2 is: H, N, Q, P, D, L, R, or Y;
   (iii) X3 is: L, H, I, R, or V; and
   (iv) X4 is Y; and/or
   wherein the AAV capsid variant comprises an amino acid modification, e.g., a conservative substitution, of any of the aforesaid amino acids in (i)-(iv);
   optionally wherein the AAV capsid variant further comprises:
   (i) one, two, or all of an amino acid other than T at position 593 (e.g., a V, S, L, R, I, A, N, C, Q, M, P, or K), an amino acid other than G at position 594 (e.g., T, M, A, K, S, Q, V, I, R, N, P, L, H, or Y), and/or an amino acid other than W at position 595 (e.g., K, Q, S, P, C, A, G, N, T, R, V, M, H, L, E, F, or Y), relative to a reference sequence numbered according to the amino acid sequence of SEQ ID NO: 138; and/or
   (ii) one, two, or all of an amino acid other than V at position 596 (e.g., a D, F, A, E, L, G, or I), an amino acid other than Q at position 597 (e.g., P, K, R, L, H, or E), and/or an amino acid other than N at position 598 (e.g., H, S, T, P, K, I, D, or Y), relative to a reference sequence numbered according to the amino acid sequence of SEQ ID NO: 138.
133. The isolated AAV particle of embodiment 132, wherein:
   (i) X1 is V;
   (ii) X2 is H;
   (iii) X3 is L, H, or I; and
   (iv) X4 is Y.
134. The isolated AAV particle of embodiment 132 or 133, wherein [B] comprises:
   (i) VH, VN, VQ, IH, LH, VP, VD, AH, FH, DH, VL, GH, VR, VY, LY, HY, IY, RY, HL, HH, HI, NL, QL, PL, DL, HR, LL, RL, HV, or YL; or
   (ii) VH, LY, HY, IY, HL, HH, or HI.
135. The isolated AAV particle of any one of embodiments 132-134, wherein [B] comprises:
   (i) VHL, VHH, VHI, VNL, VQL, IHL, LHL, VPL, VDL, AHL, VHR, FHL, DHL, VLL, GHL, VRL, VHV, VYL, HLY, HHY, HIY, NLY, QLY, PLY, DLY, HRY, LLY, RLY, HVY, YLY;
   (ii) VHL, VHH, VHI, HLY, HHY, or HIY.
136. The isolated AAV particle of any one of embodiments 132-135, wherein [B] is:

(i)
(SEQ ID NO: 4680)
VHLY,
(SEQ ID NO: 4683)
VHHY,
(SEQ ID NO: 4681)
VHIY,
(SEQ ID NO: 4724)
VNLY,
(SEQ ID NO: 4729)
VQLY,
(SEQ ID NO: 4730)
IHLY,
(SEQ ID NO: 4727)
LHLY,
(SEQ ID NO: 4723)
VPLY,
(SEQ ID NO: 4731)
VDLY,
(SEQ ID NO: 4732)
AHLY,
(SEQ ID NO: 4725)
VHRY,
(SEQ ID NO: 4726)
FHLY,
(SEQ ID NO: 4728)
DHLY,
(SEQ ID NO: 4733)
VLLY,
(SEQ ID NO: 4734)
GHLY,
(SEQ ID NO: 4735)
VRLY,
(SEQ ID NO: 4682)
VHVY,
or
(SEQ ID NO: 4736)
VYLY;
or
(ii)
(SEQ ID NO: 4680)
VHLY,
(SEQ ID NO: 4683)
VHHY,
or
(SEQ ID NO: 4681)
VHIY.

137. The isolated AAV particle of any one of embodiments 132-136, wherein [B] is or comprises VHLY (SEQ ID NO: 4680).
138. The isolated AAV particle of any one of embodiments 132-137, wherein [A][B] comprises:

(i)
(SEQ ID NO: 3681)
PLNGAVH,
(SEQ ID NO: 5110)
PLNGAVN,
(SEQ ID NO: 5111)
PLNGAVQ,
(SEQ ID NO: 5112)
PLNGAIH,
(SEQ ID NO: 5113)
PLNGALH,
(SEQ ID NO: 5114)
PLNGAVP,
(SEQ ID NO: 5115)
PLNGAVD,
(SEQ ID NO: 5116)
PLNGAAH,
(SEQ ID NO: 5117)
PLNGAFH,
(SEQ ID NO: 5118)
PLNGADH,
(SEQ ID NO: 5119)
PLNGAVL,
(SEQ ID NO: 5120)
PLNGAGH,
(SEQ ID NO: 5121)
PLNGAVR,
or
(SEQ ID NO: 5122)
PLNGAVY;
or
(ii)
(SEQ ID NO: 3681)
PLNGAVH;

(iii) amino acid sequence comprising any portion of an amino acid sequence in (i) or (ii), e.g., any 2, 3, 4, 5, or 6 amino acids, e.g., consecutive amino acids, thereof;
(iv) an amino acid sequence comprising at least one, two, or three but no more than four modifications, e.g., substitutions (e.g., conservative substitutions), insertions, or deletions, relative to any of the amino acid sequences in (i) or (ii); or
(v) an amino acid sequence comprising at least one, two, or three but no more than four different amino acids, relative to any one of the amino acid sequences in (i) or (ii).
139. The isolated AAV particle of any one of embodiments 132-138, wherein [A][B] is or comprises:

(i)
(SEQ ID NO: 3648)
PLNGAVHLY,
(SEQ ID NO: 4796)
PLNGAVHHY,
(SEQ ID NO: 4794)
PLNGAVHIY,
(SEQ ID NO: 5123)
PLNGAVNLY,
(SEQ ID NO: 5124)
PLNGAVQLY,
(SEQ ID NO: 5125)
PLNGAIHLY,
(SEQ ID NO: 5126)
PLNGALHLY,
(SEQ ID NO: 5127)
PLNGAVPLY,
(SEQ ID NO: 5128)
PLNGAVDLY,
(SEQ ID NO: 5129)
PLNGAAHLY,
(SEQ ID NO: 5130)
PLNGAVHRY,
(SEQ ID NO: 5131)
PLNGAFHLY,
(SEQ ID NO: 5132)
PLNGADHLY,
(SEQ ID NO: 5133)
PLNGAVLLY,
(SEQ ID NO: 5134)
PLNGAGHLY,
(SEQ ID NO: 5135)
PLNGAVRLY,
(SEQ ID NO: 5136)
PLNGAVHVY,
or
(SEQ ID NO: 5137)
PLNGAVYLY;
(ii)
(SEQ ID NO: 3648)
PLNGAVHLY,
(SEQ ID NO: 4796)
PLNGAVHHY,
or
(SEQ ID NO: 4794)
PLNGAVHIY;

(iii) amino acid sequence comprising any portion of an amino acid sequence in (i) or (ii), e.g., any 2, 3, 4, 5, 6, 7, or 8 amino acids, e.g., consecutive amino acids, thereof;
(iv) an amino acid sequence comprising at least one, two, or three but no more than four modifications, e.g., substitutions (e.g., conservative substitutions), insertions, or deletions, relative to any of the amino acid sequences in (i) or (ii); or
(v) an amino acid sequence comprising at least one, two, or three but no more than four different amino acids, relative to any one of the amino acid sequences in (i) or (ii).
140. The isolated AAV particle of any one of embodiments 132-139, wherein [A][B] is or comprises PLNGAVHLY (SEQ ID NO: 3648).
141. The isolated AAV particle of any one of embodiments 132-140, wherein the AAV capsid variant further comprises one, two, three, or all of an amino acid other than A at position 589 (e.g., D, S, or T), an amino acid other than Q at position 590 (e.g., K, H, L, P, or R), an amino acid other than A at position 591 (e.g., P or E), and/or an amino acid other than Q at position 592 (e.g., H, K, or P).
142. The isolated AAV particle of any one of embodiments 132-141, which further comprises one, two, three, or all of an amino acid other than A at position 596 (e.g., D, S, or T), an amino acid other than Q at position 597 (e.g., K, H, L, P, or R), an amino acid other than A at position 598 (e.g., P or E), and/or an amino acid other than Q at position 599 (e.g., H, K, or P), relative to a reference sequence numbered according to the amino acid sequence of SEQ ID NO: 5, 8, or 3636.
143. The isolated AAV particle of any one of embodiments 132-142, which further comprises:
   (i) A at position 589, Q at position 590, A at position 591, and/or Q at position 592, numbered according to the amino acid sequence of SEQ ID NO: 138; or
   (ii) A at position 596, Q at position 597, A at position 598, and/or Q at position 599, numbered according to the amino acid sequence of SEQ ID NO: 5, 8, or 3636.
144. The isolated AAV particle of any one of embodiments 132-143, wherein the AAV capsid variant further comprises [C], wherein [C] comprises X4, X5, X6, and X7, wherein:
   (a) position X4 is: A, D, S, or T;
   (b) position X5 is: Q, K, H, L, P, or R;
   (c) position X6 is: A, P, or E; and
   (d) position X7 is: Q, H, K, or P; and/or an amino acid modification, e.g., a conservative substitution, of any of the aforesaid amino acids in (a)-(d).
145. The isolated AAV particle of embodiment 144, wherein:
   (a) position X4 is: A, D, or S;
   (b) position X5 is Q or K;
   (c) position X6 is A or P; and/or
   (d) position X7 is Q.
146. The isolated AAV particle of embodiment 144 or 145, wherein [C] comprises:
   (i) AQ, AK, DQ, SQ, AH, AL, AP, AR, TQ, PQ, EQ, QA, QP, KA, HA, QE, LA, PA, or RA; or
   (ii) AQ, AK, DQ, SQ, PQ, QA, QP, or KA.
147. The isolated AAV particle of any one of embodiments 144-146, wherein [C] comprises:
   (i) AQA, AQP, AKA, DQA, SQA, AHA, AQE, ALA, APA, ARA, TQA, QAQ, QPQ, KAQ, HAQ, QEQ, QAK, LAQ, PAQ, RAQ, QAH, or QAP; or
   (ii) AQA, AQP, AKA, DQA, SQA, QAQ, QPQ, or KAQ.
148. The isolated AAV article of an one of embodiments 144-147, wherein [C] is or comprises:

(i)
(SEQ ID NO: 4737)
AQAQ,
(SEQ ID NO: 4739)
AQPQ,
(SEQ ID NO: 4741)
AKAQ,
(SEQ ID NO: 4744)
DQAQ,
(SEQ ID NO: 4738)
SQAQ,
(SEQ ID NO: 4742)
AHAQ,
(SEQ ID NO: 4748)
AQEQ,
(SEQ ID NO: 4746)
AQAK,
(SEQ ID NO: 4749)
ALAQ,
(SEQ ID NO: 4745)
APAQ,
(SEQ ID NO: 4750)
ARAQ,
(SEQ ID NO: 4747)
AQAH,
(SEQ ID NO: 4743)
AQAP,
or
(SEQ ID NO: 4751)
TQAQ;
or
(ii)
(SEQ ID NO: 4737)
AQAQ,
(SEQ ID NO: 4739)
AQPQ,
(SEQ ID NO: 4741)
AKAQ,
(SEQ ID NO: 4744)
DQAQ,
or
(SEQ ID NO: 4738)
SQAQ.

149. The isolated AAV particle of any one of embodiments 144-148, wherein [C] is or comprises AQAQ (SEQ ID NO: 4737).
150. The isolated AAV particle of any one of embodiments 144-149, wherein [B][C] is or comprises:

(i)
(SEQ ID NO: 4797)
VHLYAQAQ,
(SEQ ID NO: 4804)
VHHYAQAQ,
(SEQ ID NO: 4798)
VHLYAQPQ,
(SEQ ID NO: 4800)
VHLYAKAQ,
(SEQ ID NO: 4801)
VHLYDQAQ,
(SEQ ID NO: 4799)
VHLYSQAQ,
(SEQ ID NO: 4802)
VHIYAQAQ,
(SEQ ID NO: 5138)
VHLYAHAQ,
(SEQ ID NO: 5139)
VNLYAQAQ,
(SEQ ID NO: 5140)
VQLYAQAQ,
(SEQ ID NO: 5141)
VHLYAQEQ,
(SEQ ID NO: 5142)
IHLYAQAQ,
(SEQ ID NO: 5143)
LHLYAQAQ,
(SEQ ID NO: 5144)
VPLYAQAQ,
(SEQ ID NO: 5145)
VHLYAQAK,
(SEQ ID NO: 5146)
VDLYAQAQ,
(SEQ ID NO: 5147)
AHLYAQAQ,
(SEQ ID NO: 5148)
VHRYAQAQ,
(SEQ ID NO: 5149)
FHLYAQAQ,
(SEQ ID NO: 5150)
VHLYALAQ,
(SEQ ID NO: 5151)
DHLYAQAQ,
(SEQ ID NO: 5152)
VHLYAPAQ,
(SEQ ID NO: 5153)
VHLYARAQ,
(SEQ ID NO: 5154)
VHLYAQAH,
(SEQ ID NO: 5155)
VLLYAQAQ,
(SEQ ID NO: 5156)
VHLYAQAP,
(SEQ ID NO: 5157)
GHLYAQAQ,
(SEQ ID NO: 5158)
VRLYAQAQ,
(SEQ ID NO: 4803)
VHVYAQAQ,
(SEQ ID NO: 5159)
VYLYAQAQ,
or
(SEQ ID NO: 5160)
VHLYTQAQ;
(ii)
(SEQ ID NO: 4797)
VHLYAQAQ,
(SEQ ID NO: 4804)
VHHYAQAQ,
(SEQ ID NO: 4798)
VHLYAQPQ,
(SEQ ID NO: 4800)
VHLYAKAQ,
(SEQ ID NO: 4801)
VHLYDQAQ,
(SEQ ID NO: 4799)
VHLYSQAQ,
or
(SEQ ID NO: 4802)
VHIYAQAQ;

(iii) amino acid sequence comprising any portion of an amino acid sequence in (i) or (ii), e.g., any 2, 3, 4, 5, 6, or 7 amino acids, e.g., consecutive amino acids, thereof;
(iv) an amino acid sequence comprising at least one, two, or three but no more than four modifications, e.g., substitutions (e.g., conservative substitutions), insertions, or deletions, relative to any of the amino acid sequences in (i) or (ii); or
(v) an amino acid sequence comprising at least one, two, or three but no more than four different amino acids, relative to any one of the amino acid sequences in (i) or (ii).
151. The isolated AAV particle of any one of embodiments 144-150, wherein [B][C] is or comprises VHLYAQAQ (SEQ ID NO: 4797).
152. The isolated AAV particle of any one of embodiments 144-151, wherein [A][B][C] comprises:

(i)
(SEQ ID NO: 4813)
PLNGAVHLYAQ,
(SEQ ID NO: 4826)
PLNGAVHHYAQ,
(SEQ ID NO: 4812)
PLNGAVHLYAK,
(SEQ ID NO: 4814)
PLNGAVHLYDQ,
(SEQ ID NO: 4815)
PLNGAVHLYSQ,
(SEQ ID NO: 4824)
PLNGAVHIYAQ,
(SEQ ID NO: 5161)
PLNGAVHLYAH,
(SEQ ID NO: 5162)
PLNGAVNLYAQ,
(SEQ ID NO: 5163)
PLNGAVQLYAQ,
(SEQ ID NO: 5164)
PLNGAIHLYAQ,
(SEQ ID NO: 5165)
PLNGALHLYAQ,
(SEQ ID NO: 5166)
PLNGAVPLYAQ,
(SEQ ID NO: 5167)
PLNGAVDLYAQ,
(SEQ ID NO: 5168)
PLNGAAHLYAQ,
(SEQ ID NO: 5169)
PLNGAVHRYAQ,
(SEQ ID NO: 5170)
PLNGAFHLYAQ,
(SEQ ID NO: 5171)
PLNGAVHLYAL,
(SEQ ID NO: 5172)
PLNGADHLYAQ,
(SEQ ID NO: 5173)
PLNGAVHLYAP,
(SEQ ID NO: 5174)
PLNGAVHLYAR,
(SEQ ID NO: 5175)
PLNGAVLLYAQ,
(SEQ ID NO: 5176)
PLNGAGHLYAQ,
(SEQ ID NO: 5177)
PLNGAVRLYAQ,
(SEQ ID NO: 5178)
PLNGAVHVYAQ,
(SEQ ID NO: 5179)
PLNGAVYLYAQ,
(SEQ ID NO: 5180)
PLNGAVHLYTQ;
(ii)
(SEQ ID NO: 4813)
PLNGAVHLYAQ,
(SEQ ID NO: 4826)
PLNGAVHHYAQ,
(SEQ ID NO: 4812)
PLNGAVHLYAK,
(SEQ ID NO: 4814)
PLNGAVHLYDQ,
(SEQ ID NO: 4815)
PLNGAVHLYSQ,
(SEQ ID NO: 4824)
PLNGAVHIYAQ;

(iii) amino acid sequence comprising any portion of an amino acid sequence in (i) or (ii), e.g., any 2, 3, 4, 5, 6, 7, 8, 9, or 10 amino acids, e.g., consecutive amino acids, thereof;
(iv) an amino acid sequence comprising at least one, two, or three but no more than four modifications, e.g., substitutions (e.g., conservative substitutions), insertions, or deletions, relative to any of the amino acid sequences in (i) or (ii); or
(v) an amino acid sequence comprising at least one, two, or three but no more than four different amino acids, relative to any one of the amino acid sequences in (i) or (ii).
153. The isolated AAV particle of any one of embodiments 144-152, wherein [A][B][C] is or comprises:

(i)
(SEQ ID NO: 4836)
PLNGAVHLYAQAQ,
(SEQ ID NO: 4850)
PLNGAVHHYAQAQ,
(SEQ ID NO: 4837)
PLNGAVHLYAQPQ,
(SEQ ID NO: 4835)
PLNGAVHLYAKAQ,
(SEQ ID NO: 4838)
PLNGAVHLYDQAQ,
(SEQ ID NO: 4839)
PLNGAVHLYSQAQ,
(SEQ ID NO: 4848)
PLNGAVHIYAQAQ,
(SEQ ID NO: 5181)
PLNGAVHLYAHAQ,
(SEQ ID NO: 5182)
PLNGAVNLYAQAQ,
(SEQ ID NO: 5183)
PLNGAVQLYAQAQ,
(SEQ ID NO: 5184)
PLNGAVHLYAQEQ,
(SEQ ID NO: 5185)
PLNGAIHLYAQAQ,
(SEQ ID NO: 5186)
PLNGALHLYAQAQ,
(SEQ ID NO: 5187)
PLNGAVPLYAQAQ,
(SEQ ID NO: 5188)
PLNGAVHLYAQAK,
(SEQ ID NO: 5189)
PLNGAVDLYAQAQ,
(SEQ ID NO: 5190)
PLNGAAHLYAQAQ,
(SEQ ID NO: 5191)
PLNGAVHRYAQAQ,
(SEQ ID NO: 5192)
PLNGAFHLYAQAQ,
(SEQ ID NO: 5193)
PLNGAVHLYALAQ,
(SEQ ID NO: 5194)
PLNGADHLYAQAQ,
(SEQ ID NO: 5195)
PLNGAVHLYAPAQ,
(SEQ ID NO: 5196)
PLNGAVHLYARAQ,
(SEQ ID NO: 5197)
PLNGAVHLYAQAH,
(SEQ ID NO: 5198)
PLNGAVLLYAQAQ,
(SEQ ID NO: 5199)
PLNGAVHLYAQAP,
(SEQ ID NO: 5200)
PLNGAGHLYAQAQ,
(SEQ ID NO: 5201)
PLNGAVRLYAQAQ,
(SEQ ID NO: 5202)
PLNGAVHVYAQAQ,
(SEQ ID NO: 5203)
PLNGAVYLYAQAQ,
(SEQ ID NO: 5204)
PLNGAVHLYTQAQ;
(ii)
(SEQ ID NO: 4836)
PLNGAVHLYAQAQ,
(SEQ ID NO: 4850)
PLNGAVHHYAQAQ,
(SEQ ID NO: 4837)
PLNGAVHLYAQPQ,
(SEQ ID NO: 4835)
PLNGAVHLYAKAQ,
(SEQ ID NO: 4838)
PLNGAVHLYDQAQ,
(SEQ ID NO: 4839)
PLNGAVHLYSQAQ,
(SEQ ID NO: 4848)
PLNGAVHIYAQAQ;

(iii) amino acid sequence comprising any portion of an amino acid sequence in (i) or (ii), e.g., any 2, 3, 4, 5, 6, 7, 8, 9, 10, 11, or 12 amino acids, e.g., consecutive amino acids, thereof;
(iv) an amino acid sequence comprising at least one, two, or three but no more than four modifications, e.g., substitutions (e.g., conservative substitutions), insertions, or deletions, relative to any of the amino acid sequences in (i) or (ii); or
(v) an amino acid sequence comprising at least one, two, or three but no more than four different amino acids, relative to any one of the amino acid sequences in (i) or (ii).
154. The isolated AAV particle of any one of embodiments 144-153, wherein [A][B][C] is or comprises PLNGAVHLYAQAQ (SEQ ID NO: 4836).
155. The isolated AAV particle of any one of embodiment 132-154, wherein the AAV capsid variant further comprises one, two, or all of an amino acid other than T at position 593 (e.g., a V, S, L, R, I, A, N, C, Q, M, P, or K), an amino acid other than G at position 594 (e.g., T, M, A, K, S, Q, V, I, R, N, P, L, H, or Y), and/or an amino acid other than W at position 595 (e.g., K, Q, S, P, C, A, G, N, T, R, V, M, H, L, E, F, or Y), relative to a reference sequence numbered according to the amino acid sequence of SEQ ID NO: 138
156. The isolated AAV particle of any one of embodiment 132-155, which further comprises one, two, or all of an amino acid other than T at position 600 (e.g., a V, S, L, R, I, A, N, C, Q, M, P, or K), an amino acid other than G at position 601 (e.g., T, M, A, K, S, Q, V, I, R, N, P, L, H, or Y), and/or an amino acid other than W at position 602 (e.g., K, Q, S, P, C, A, G, N, T, R, V, M, H, L, E, F, or Y), relative to a reference sequence numbered according to the amino acid sequence of SEQ ID NO: 5, 8, 3636.
157. The isolated AAV particle of any one of embodiment 132-156, which further comprises one, two, three or all of:
   (i) the amino acid V, S, L, R, I, A, N, C, Q, M, P, or K (e.g., L) at position 593 numbered according to SEQ ID NO: 138 or at position 600 numbered according to SEQ ID NO: 5, 8, or 3636);
   (ii) the amino acid T, M, A, K, S, Q, V, I, R, N, P, L, H, or Y (e.g., S) at position 594 numbered according to SEQ ID NO: 138, or at position 601 numbered according to SEQ ID NO: 5, 8, or 3636; and/or
   (iii) the amino acid K, Q, S, P, C, A, G, N, T, R, V, M, H, L, E, F, or Y (e.g. P) at position 595 numbered according to SEQ ID NO: 138 or at position 602 numbered according to SEQ ID NO: 5, 8, or 3636).
158. The isolated AAV particle of any one of embodiment 132-157, which further comprises:
   (i) the amino acid L at position 593 numbered according to SEQ ID NO: 138 or at position 600 numbered according to SEQ ID NO: 5, 8, or 3636);
   (ii) the amino acid S at position 594 numbered according to SEQ ID NO: 138, or at position 601 numbered according to SEQ ID NO: 5, 8, or 3636; and
   (iii) the amino acid P at position 595 numbered according to SEQ ID NO: 138 or at position 602 numbered according to SEQ ID NO: 5, 8, or 3636).
159. The isolated AAV particle of any one of embodiment 132-158, which further comprises:
   (i) T at position 593, G at position 594, and/or W at position 595, numbered according to SEQ ID NO: 138;
   (ii) T at position 600, G at position 601, and/or W at position 602, numbered according to SEQ ID NO: 5, 8, or 3636.
160. The isolated AAV particle of any one of embodiments 132-159, wherein the AAV capsid variant further comprises [D], wherein [D] comprises X8, X9, and X10, wherein:
   (a) position X8 is: T, V, S, L, R, I, A, N, C, Q, M, P, or K;
   (b) position X9 is: T, M, A, G, K, S, Q, V, I, R, N, P, L, H, or Y; and
   (c) position X10 is: K, Q, W, S, P, C, A, G, N, T, R, V, M, H, L, E, F, or Y; and/or an amino acid modification, e.g., a conservative substitution, of any of the aforesaid amino acids in (a)-(c).
161. The isolated AAV particle of embodiment 160, wherein:
   (a) position X8 is: T, V, S, L, R, I, A, N, C, Q, or M;
   (b) position X9 is: T, M, A, G, K, S, Q, V, I, R, N, P, L, or H; and/or
   (c) position X10 is: K, Q, W, S, P, C, A, G, N, T, R, V, M, H, L, or E.
162. The isolated AAV particle of embodiment 160 or 161, wherein [D] comprises:
   (i) TT, TM, VA, TA, TG, VK, SA, LS, LA, TQ, TV, RI, RA, LT, ST, TS, VS, VT, RQ, IS, VR, LG, TN, VQ, AA, RS, IQ, IA, RG, NS, LQ, VM, SM, VG, CS, TP, SS, AG, TL, LN, TK, CT, AS, LK, LM, LH, RT, RM, VH, TR, SG, VL, QA, NA, AT, NT, RL, IT, IG, RN, NM, NV, MA, IL, VN, SV, RV, PG, QS, RY, SQ, NQ, LL, LP, AQ, TY, NL, SP, LV, KG, VP, AV, KS, AM, SL, AL, RP, IP, MK, AW, GS, KQ, AP, SK, AK, GC, QK, MQ, QP, GP, QQ, AN, GK, QR, PP, AR, GG, MS, NP, KP, MN, KA, SN, MP, HP, GN, RW, MT, SR, GW, QH, GL, QM, VW, MG, AH, QT, GR, SH, GQ, GT, GA, NG, QN, VE, MM, QL, QG, YS, GM, LR, AF, PQ, SW, QW, YA, ML, GF, PA, PS, PT, GY, GV, PW, PR; or
   (ii) TT, TM, VA, TA, TG, VK, SA, LS, LA, TQ, TV, RI, RA, LT, ST, TS, VS, VT, RQ, IS, VR, LG, TN, VQ, AA, RS, IQ, IA, RG, NS, LQ, VM, SM, VG, CS, TP, SS, AG, TL, LN, TK, CT, AS, LK, LM, LH, RT, RM, VH, TR, SG, VL, QA, NA, AT, NT, RL, IT, IG, RN, NM, NV, MA, IL, VN, SV, RV, MK, AQ, AW, GS, KQ, AP, SK, AK, GC, QK, SP, MQ, SQ, QP, RP, GP, NQ, QQ, AN, GK, QS, QR, PP, AR, GG, MS, NP, KP, MN, KS, KA, SN, MP, HP, GN, RW, MT, AM, SR, GW, QH, GL, AV, QM, VW, MG, AL, AH, SL, QT, GR, SH, LP, GQ, GT, GA, NG, QN, IP, or VE.
163. The isolated AAV particle of any one of embodiments 160-162, wherein [D] is or comprises:
   (i) TTK, TMK, VAQ, TAW, TGS, VKQ, SAP, LSK, LAP, LAQ, TAK, SAK, TGC, TQK, TVA, LSP, TTQ, TAQ, RIA, RAS, TTP, LTP, STP, TSP, TMQ, TSK, VSQ, VSP, TVQ, VTA, RQP, ISG, VRP, LGP, TNQ, VQQ, VAN, AAP, RST, TMA, IQP, IAS, TVS, RGS, NSP, LQP, VTG, VMQ, SMA, VGK, IQS, CSP, LQR, TPP, VTK, SSP, AGP, LAR, TTT, TGG, TLQ, TMS, VAK, RAA, TVG, LNP, LSQ, TKP, TNA, LAT, VTP, VQA, TTS, CTP, TAG, TSQ, TMN, TST, VKP, ASP, VAA, LKS, IAA, TAA, TKA, VSN, TAP, LMP, LHP, RAQ, LTN, RTT, TSV, RMS, VGN, LMQ, TAT, VHP, ISS, VAS, TRW, TMT, RSS, RTG, VAT, VTS, VSS, TNS, VKA, SGP, TGP, TAM, TQP, TQQ, VSR, TGW, VSA, VLS, TQH, LAS, QAP, NAQ, ATP, VQP, TTA, LAA, RSG, LMA, TMP, LAN, VST, SAQ, NTP, TGL, TAV, RLG, RTL, TQM, ITP, TVW, RSA, TAS, TMG, VQS, ISP, VGG, TAL, LAG, RTA, RSP, TLA, LAH, TSL, RLS, LMG, SMQ, TQT, VGS, VSG, VMA, IGG, IAG, TGR, LSH, VQT, RNS, TLP, TKQ, LGQ, NMQ, NVQ, RGG, VMS, TTG, LSR, MAP, ILG, TGT, TSS, TSH, RIG, SAM, TSM, SMG, SMS, TSG, TGA, VNS, VAG, IGS, LGS, VNG, LTA, VQN, TKS, SVG, NAS, TSA, TAN, LTS, RSQ, RIP, RVE, VLP, SVA, LQQ, LST, SAA, RTS, TQN, VNA, LMS, TMM, RSV, TQL, RTP, RQQ, VQG, PGW, STQ, QSP, RYS, TQR, SAG, RQS, SQP, STS, VLG, NQP, LGT, RAG, TGM, LSN, RLP, RQG, RLT, TLR, SAF, SVQ, LLP, RTQ, LPP, AQP, TPQ, TSW, NTT, TTR, TQW, NTQ, TYA, TLS, NLP, ATS, ATQ, LSS, TQA, VMP, NAL, RML, RQL, TLG, TGF, SAL, SQL, LSA, TGQ, TNG, AAA, SAV, LSG, SSR, SPP, LVG, TPA, KGW, VPP, ATG, SAN, SQQ, SSM, AVG, VAP, TPS, RGW, SSL, TYS, TPT, IGW, KSS, TGY, RSL, SVS, TSN, SQM, VPA, AMS, TPG, TGV, VPQ, SLP, ALP, TPW, TPR, SSS, RPP, IPP, AGW, or RPG; or
   (ii) TTK, TMK, VAQ, TAW, TGS, VKQ, SAP, LSK, LAP, LAQ, TAK, SAK, TGC, TQK, TVA, LSP, TTQ, TAQ, RIA, RAS, TTP, LTP, STP, TSP, TMQ, TSK, VSQ, VSP, TVQ, VTA, RQP, ISG, VRP, LGP, TNQ, VQQ, VAN, AAP, RST, TMA, IQP, IAS, TVS, RGS, NSP, LQP, VTG, VMQ, SMA, VGK, IQS, CSP, LQR, TPP, VTK, SSP, AGP, LAR, TTT, TGG, TLQ, TMS, VAK, RAA, TVG, LNP, LSQ, TKP, TNA, LAT, VTP, VQA, TTS, CTP, TAG, TSQ, TMN, TST, VKP, ASP, VAA, LKS, IAA, TAA, TKA, VSN, TAP, LMP, LHP, RAQ, LTN, RTT, TSV, RMS, VGN, LMQ, TAT, VHP, ISS, VAS, TRW, TMT, RSS, RTG, VAT, VTS, VSS, TNS, VKA, SGP, TGP, TAM, TQP, TQQ, VSR, TGW, VSA, VLS, TQH, LAS, QAP, NAQ, ATP, VQP, TTA, LAA, RSG, LMA, TMP, LAN, VST, SAQ, NTP, TGL, TAV, RLG, RTL, TQM, ITP, TVW, RSA, TAS, TMG, VQS, ISP, VGG, TAL, LAG, RTA, RSP, TLA, LAH, TSL, RLS, LMG, SMQ, TQT, VGS, VSG, VMA, IGG, IAG, TGR, LSH, VQT, RNS, TLP, TKQ, LGQ, NMQ, NVQ, RGG, VMS, TTG, LSR, MAP, ILG, TGT, TSS, TSH, RIG, SAM, TSM, SMG, SMS, TSG, TGA, VNS, VAG, IGS, LGS, VNG, LTA, VQN, TKS, SVG, NAS, TSA, TAN, LTS, RSQ, RIP, RVE, VLP, SVA, LQQ, LST, SAA, RTS, TQN, VNA, or LMS.
164. The isolated AAV particle of any one of embodiments 160-163, wherein [D] is or comprises TGW.
165. The isolated AAV particle of any one of embodiments 160-163, wherein [D] is or comprises LSP.
166. The isolated AAV particle of any one of embodiments 132-164, wherein the AAV capsid variant further comprises one, two, or all of an amino acid other than V at position 596 (e.g., D, F, A, E, L, G, or I), an amino acid other than Q at position 597 (e.g., R, P, K, L, H, or E), and/or an amino acid other than N at position 598 (e.g., H, S, T, P, K, I, D, or Y), relative to a reference sequence numbered according to the amino acid sequence of SEQ ID NO: 138.
167. The isolated AAV particle of any one of embodiments 132-166, which further comprises one, two, or all of an amino acid other than V at position 603 (e.g., D, F, A, E, L, G, or I), an amino acid other than Q at position 604 (e.g., R, P, K, L, H, or E), and/or an amino acid other than N at position 605 (e.g., H, S, T, P, K, I, D, or Y), relative to a reference sequence numbered according to the amino acid sequence of SEQ ID NO: 5, 8, or 3636.
168. The isolated AAV particle of any one of embodiments 132-167, which further comprises one, two, or all of:
   (i) the amino acid D, F, A, E, L, G, or I at position 596, numbered according to SEQ ID NO: 138, or at position 603 numbered according to SEQ ID NO: 5, 8, or 3636;
   (ii) the amino acid R, P, K, L, H, or E at position 597, numbered according to SEQ ID NO: 138, or at position 604 numbered according to SEQ ID NO: 5, 8, or 3636; and/or
   (iii) the amino acid H, S, T, P, K, I, D, or Y at position 598, numbered according to SEQ ID NO: 138, or at position 605 numbered according to SEQ ID NO: 5, 8, or 3636.
169. The isolated AAV particle of any one of embodiments 132-168, which further comprises P, K, E, or H at position 597, numbered according to SEQ ID NO: 138.
170. The isolated AAV particle of any one of embodiments 132-169, which further comprises one, two, or all of:
   (i) the amino acid V at position 596 numbered according to SEQ ID NO: 138 or at position 603 numbered according to SEQ ID NO: 5, 8, or 3636;
   (ii) the amino acid K, P, E or H at position 597 numbered according to SEQ ID NO: 138 or at position 604 numbered according to SEQ ID NO: 5, 8, or 3636; and
   (iii) the amino acid N at position 598 numbered according to the amino acid sequence of SEQ ID NO: 138 or at position 605 numbered according to SEQ ID NO: 5, 8, or 3636.
171. The isolated AAV particle of any one of embodiments 132-170, wherein the AAV capsid variant further comprises [E], wherein [E] comprises X11, X12, and X13, wherein:
   (a) position X11 is: V, D, F, A, E, L, G, or I;
   (b) position X12 is: Q, R, P, K, L, H, or E; and
   (c) position X13 is: N, H, S, T, P, K, I, D, or Y; and/or an amino acid modification, e.g., a conservative substitution, of any of the aforesaid amino acids in (a)-(c).
172. The isolated AAV particle of embodiment 171, wherein:
   (a) position X11 is: V, D, F, A, E, L, or G;
   (b) position X12 is: Q, R, P, K, or L; and/or
   (c) position X13 is: N, H, S, T, P, K, I, or D.
173. The isolated AAV particle of embodiment 171 or 172, wherein:
   (a) position X11 is V;
   (b) position X12: Q, R, P, K, or L; and/or
   (c) position X13 is: N.
174. The isolated AAV particle of any one of embodiments 171-173, wherein position X12 is P.
175. The isolated AAV particle of any one of embodiments 171-174, wherein position X12 is K.
176. The isolated AAV particle of any one of embodiments 171-175, wherein position X12 is E or H.
177. The isolated AAV particle of any one of embodiments 171-176, wherein [E] comprises:
   (i) VQ, DQ, FQ, VR, VP, VK, AQ, EQ, LQ, GQ, VL, VH, VE, DK, GH, IQ, QN, QH, QS, QT, QP, RN, PN, KN, QK, QI, LN, QD, HN, KT, KK, EN, QY, or PH; or
   (ii) VQ, DQ, FQ, VR, VP, VK, AQ, EQ, LQ, GQ, VL, QN, QH, QS, QT, QP, RN, PN, KN, QK, QI, LN, or QD.
178. The isolated AAV particle of any one of embodiments 171-177, wherein [E] is or comprises:
   (i) VQN, DQN, VQH, FQN, VQS, VQT, VQP, VRN, VPN, VKN, AQN, VQK, EQN, VQI, LQN, GQT, VLN, VQD, VHN, GQN, VKT, VKK, FQK, VEN, VQY, DKN, GHN, IQN, or VPH; or
   (ii) VQN, DQN, VQH, FQN, VQS, VQT, VQP, VRN, VPN, VKN, AQN, VQK, EQN, VQI, LQN, GQT, VLN, or VQD.
179. The isolated AAV particle of any one of embodiments 171-178, wherein [E] is or comprises VQN, VPN, or VKN.
180. The isolated AAV particle of any one of embodiments 171-178, wherein [E] is or comprises VEN, VHN, VKN, or VPN.
181. The isolated AAV particle of any one of embodiments 171-180, wherein [D][E] is or comprises:

(i)
(SEQ ID NO: 5047)
TTKVQN,
(SEQ ID NO: 5013)
TMKVQN,
(SEQ ID NO: 5062)
VAQVQN,
(SEQ ID NO: 4978)
TAWDQN,
(SEQ ID NO: 4992)
TGSVQH,
(SEQ ID NO: 5072)
VKQVQN,
(SEQ ID NO: 4854)
SAPVQN,
(SEQ ID NO: 4912)
LSKVQN,
(SEQ ID NO: 4891)
LAPVQN,
(SEQ ID NO: 4893)
LAQVQN,
(SEQ ID NO: 4968)
TAKVQN,
(SEQ ID NO: 4955)
SAKVQN,
(SEQ ID NO: 4982)
TGCFQN,
(SEQ ID NO: 5026)
TQKVQN,
(SEQ ID NO: 5052)
TVAVQN,
(SEQ ID NO: 4914)
LSPVQN,
(SEQ ID NO: 5050)
TTQVQN,
(SEQ ID NO: 4973)
TAQVQN,
(SEQ ID NO: 4934)
RIAVQN,
(SEQ ID NO: 4931)
RASVQN,
(SEQ ID NO: 5048)
TTPVQN,
(SEQ ID NO: 4853)
LTPVQN,
(SEQ ID NO: 4963)
STPVQN,
(SEQ ID NO: 4861)
TSPVQN,
(SEQ ID NO: 5016)
TMQVQN,
(SEQ ID NO: 5036)
TSKVQN,
(SEQ ID NO: 5092)
VSQVQN,
(SEQ ID NO: 4868)
VSPVQN,
(SEQ ID NO: 5055)
TVQVQN,
(SEQ ID NO: 5097)
VTAVQN,
(SEQ ID NO: 4941)
RQPVQN,
(SEQ ID NO: 4884)
ISGVQN,
(SEQ ID NO: 5087)
VRPVQN,
(SEQ ID NO: 4897)
LGPVQN,
(SEQ ID NO: 5022)
TNQVQN,
(SEQ ID NO: 5084)
VQQVQN,
(SEQ ID NO: 5061)
VANVQN,
(SEQ ID NO: 4870)
AAPVQN,
(SEQ ID NO: 4947)
RSTVQN,
(SEQ ID NO: 5011)
TMAVQN,
(SEQ ID NO: 4882)
IQPVQN,
(SEQ ID NO: 4878)
IASVQN,
(SEQ ID NO: 4867)
TVSVQN,
(SEQ ID NO: 4933)
RGSVQN,
(SEQ ID NO: 4925)
NSPVQN,
(SEQ ID NO: 4908)
LQPVQN,
(SEQ ID NO: 5098)
VTGVQN,
(SEQ ID NO: 5076)
VMQVQN,
(SEQ ID NO: 4959)
SMAVQN,
(SEQ ID NO: 5066)
VGKVQN,
(SEQ ID NO: 4883)
IQSVQN,
(SEQ ID NO: 4874)
CSPVQN,
(SEQ ID NO: 4910)
LQRVQN,
(SEQ ID NO: 4979)
TAWVQH,
(SEQ ID NO: 5024)
TPPVQN,
(SEQ ID NO: 5099)
VTKVQN,
(SEQ ID NO: 4855)
SSPVQN,
(SEQ ID NO: 4871)
AGPVQN,
(SEQ ID NO: 4894)
LARVQN,
(SEQ ID NO: 5051)
TTTVQN,
(SEQ ID NO: 4984)
TGGFQN,
(SEQ ID NO: 5010)
TLQVQN,
(SEQ ID NO: 5018)
TMSVQN,
(SEQ ID NO: 5060)
VAKVQN,
(SEQ ID NO: 4929)
RAAVQN,
(SEQ ID NO: 5054)
TVGVQN,
(SEQ ID NO: 4907)
LNPVQN,
(SEQ ID NO: 4915)
LSQVQN,
(SEQ ID NO: 5006)
TKPVQN,
(SEQ ID NO: 5021)
TNAVQN,
(SEQ ID NO: 4896)
LATVQN,
(SEQ ID NO: 5100)
VTPVQN,
(SEQ ID NO: 5081)
VQAVQN,
(SEQ ID NO: 4860)
TTSVQN,
(SEQ ID NO: 4875)
CTPVQN,
(SEQ ID NO: 4967)
TAGVQN,
(SEQ ID NO: 5040)
TSQVQN,
(SEQ ID NO: 5014)
TMNVQN,
(SEQ ID NO: 5043)
TSTVQN,
(SEQ ID NO: 5071)
VKPVQN,
(SEQ ID NO: 4872)
ASPVQN,
(SEQ ID NO: 5058)
VAAVQN,
(SEQ ID NO: 4901)
LKSVQN,
(SEQ ID NO: 4876)
IAAVQN,
(SEQ ID NO: 4966)
TAAVQN,
(SEQ ID NO: 5005)
TKAVQN,
(SEQ ID NO: 4994)
TGSVQS,
(SEQ ID NO: 5090)
VSNVQN,
(SEQ ID NO: 4971)
TAPVQN,
(SEQ ID NO: 4904)
LMPVQN,
(SEQ ID NO: 4900)
LHPVQN,
(SEQ ID NO: 4930)
RAQVQN,
(SEQ ID NO: 4919)
LTNVQN,
(SEQ ID NO: 4952)
RTTVQN,
(SEQ ID NO: 5044)
TSVVQN,
(SEQ ID NO: 4939)
RMSVQN,
(SEQ ID NO: 5067)
VGNVQN,
(SEQ ID NO: 4905)
LMQVQN,
(SEQ ID NO: 4976)
TATVQN,
(SEQ ID NO: 5069)
VHPVQN,
(SEQ ID NO: 5091)
VSPVQT,
(SEQ ID NO: 4886)
ISSVQN,
(SEQ ID NO: 5063)
VASVQN,
(SEQ ID NO: 5032)
TRWDQN,
(SEQ ID NO: 5020)
TMTVQN,
(SEQ ID NO: 4946)
RSSVQN,
(SEQ ID NO: 4863)
TAWVQN,
(SEQ ID NO: 4949)
RTGVQN,
(SEQ ID NO: 5064)
VATVQN,
(SEQ ID NO: 5101)
VTSVQN,
(SEQ ID NO: 4869)
VSSVQN,
(SEQ ID NO: 5023)
TNSVQN,
(SEQ ID NO: 5070)
VKAVQN,
(SEQ ID NO: 4958)
SGPVQN,
(SEQ ID NO: 4989)
TGPVQN,
(SEQ ID NO: 4969)
TAMVQN,
(SEQ ID NO: 5029)
TQPVQN,
(SEQ ID NO: 5030)
TQQVQN,
(SEQ ID NO: 5093)
VSRVQN,
(SEQ ID NO: 5002)
TGWVQP,
(SEQ ID NO: 5088)
VSAVQN,
(SEQ ID NO: 5074)
VLSVQN,
(SEQ ID NO: 5025)
TQHVQN,
(SEQ ID NO: 4895)
LASVQN,
(SEQ ID NO: 4928)
QAPVQN,
(SEQ ID NO: 4922)
NAQVQN,
(SEQ ID NO: 4873)
ATPVQN,
(SEQ ID NO: 5083)
VQPVQN,
(SEQ ID NO: 5045)
TTAVQN,
(SEQ ID NO: 5004)
TGWVRN,
(SEQ ID NO: 4852)
LAAVQN,
(SEQ ID NO: 5039)
TSPDQN,
(SEQ ID NO: 4943)
RSGVQN,
(SEQ ID NO: 4988)
TGGVQT,
(SEQ ID NO: 5000)
TGWVPN,
(SEQ ID NO: 5049)
TTPVQT,
(SEQ ID NO: 4902)
LMAVQN,
(SEQ ID NO: 5015)
TMPVQN,
(SEQ ID NO: 4890)
LANVQN,
(SEQ ID NO: 5096)
VSTVQN,
(SEQ ID NO: 4957)
SAQVQN,
(SEQ ID NO: 4926)
NTPVQN,
(SEQ ID NO: 5095)
VSSVQT,
(SEQ ID NO: 5056)
TVSVKN,
(SEQ ID NO: 5102)
TGLVQN,
(SEQ ID NO: 4865)
TGSVQN,
(SEQ ID NO: 4983)
TGGAQN,
(SEQ ID NO: 4977)
TAVVQN,
(SEQ ID NO: 4937)
RLGVQN,
(SEQ ID NO: 4950)
RTLVQN,
(SEQ ID NO: 5027)
TQMVQN,
(SEQ ID NO: 4887)
ITPVQN,
(SEQ ID NO: 5057)
TVWVQK,
(SEQ ID NO: 4942)
RSAVQN,
(SEQ ID NO: 4974)
TASVQN,
(SEQ ID NO: 5012)
TMGVQN,
(SEQ ID NO: 4986)
TGGVQH,
(SEQ ID NO: 5085)
VQSVQN,
(SEQ ID NO: 4864)
TGGVQN,
(SEQ ID NO: 4885)
ISPVQN,
(SEQ ID NO: 5103)
TGWVKN,
(SEQ ID NO: 4990)
TGSAQN,
(SEQ ID NO: 4857)
TGWAQN,
(SEQ ID NO: 5019)
TMSVQT,
(SEQ ID NO: 5065)
VGGVQN,
(SEQ ID NO: 4892)
LAPVQT,
(SEQ ID NO: 4862)
TALVQN,
(SEQ ID NO: 4888)
LAGVQN,
(SEQ ID NO: 4948)
RTAVQN,
(SEQ ID NO: 4944)
RSPVQN,
(SEQ ID NO: 4859)
TLAVQN,
(SEQ ID NO: 4889)
LAHVQN,
(SEQ ID NO: 5037)
TSLVQN,
(SEQ ID NO: 4938)
RLSVQN,
(SEQ ID NO: 4903)
LMGVQN,
(SEQ ID NO: 4961)
SMQVQN,
(SEQ ID NO: 5031)
TQTVQN,
(SEQ ID NO: 4997)
TGWEQN,
(SEQ ID NO: 5068)
VGSVQN,
(SEQ ID NO: 5089)
VSGVQN,
(SEQ ID NO: 5075)
VMAVQN,
(SEQ ID NO: 4879)
IGGVQN,
(SEQ ID NO: 4877)
IAGVQN,
(SEQ ID NO: 4856)
TGRVQN,
(SEQ ID NO: 4911)
LSHVQN,
(SEQ ID NO: 5086)
VQTVQN,
(SEQ ID NO: 4866)
TGWDQN,
(SEQ ID NO: 4940)
RNSVQN,
(SEQ ID NO: 5009)
TLPVQN,
(SEQ ID NO: 5007)
TKQVQN,
(SEQ ID NO: 4898)
LGQVQN,
(SEQ ID NO: 4924)
NMQVQN,
(SEQ ID NO: 4927)
NVQVQN,
(SEQ ID NO: 4993)
TGSVQI,
(SEQ ID NO: 4932)
RGGVQN,
(SEQ ID NO: 5077)
VMSVQN,
(SEQ ID NO: 5046)
TTGVQN,
(SEQ ID NO: 4913)
LSPVQK,
(SEQ ID NO: 4916)
LSRVQN,
(SEQ ID NO: 5094)
VSSVQK,
(SEQ ID NO: 4972)
TAPVQT,
(SEQ ID NO: 4921)
MAPVQN,
(SEQ ID NO: 4881)
ILGVQN,
(SEQ ID NO: 4975)
TASVQT,
(SEQ ID NO: 4991)
TGSLON,
(SEQ ID NO: 4996)
TGTVQN,
(SEQ ID NO: 4995)
TGSVQT,
(SEQ ID NO: 5042)
TSSVQT,
(SEQ ID NO: 5035)
TSHVQN,
(SEQ ID NO: 4935)
RIGVQN,
(SEQ ID NO: 4999)
TGWGQT,
(SEQ ID NO: 4956)
SAMVQN,
(SEQ ID NO: 5038)
TSMVQN,
(SEQ ID NO: 4960)
SMGVQN,
(SEQ ID NO: 4962)
SMSVQN,
(SEQ ID NO: 5041)
TSSVQN,
(SEQ ID NO: 5034)
TSGVQN,
(SEQ ID NO: 4981)
TGAVQN,
(SEQ ID NO: 5080)
VNSVQN,
(SEQ ID NO: 5059)
VAGVQN,
(SEQ ID NO: 4880)
IGSVQN,
(SEQ ID NO: 4899)
LGSVQN,
(SEQ ID NO: 5079)
VNGVQN,
(SEQ ID NO: 4918)
LTAVQN,
(SEQ ID NO: 5082)
VQNVQN,
(SEQ ID NO: 5008)
TKSVQN,
(SEQ ID NO: 4965)
SVGVQN,
(SEQ ID NO: 4980)
TAWVQT,
(SEQ ID NO: 4923)
NASVQN,
(SEQ ID NO: 5033)
TSAVQN,
(SEQ ID NO: 5017)
TMSVKN,
(SEQ ID NO: 4970)
TANVQN,
(SEQ ID NO: 4998)
TGWFQN,
(SEQ ID NO: 4985)
TGGVLN,
(SEQ ID NO: 4920)
LTSVQN,
(SEQ ID NO: 5003)
TGWVQT,
(SEQ ID NO: 4945)
RSQVQN,
(SEQ ID NO: 4936)
RIPVQN,
(SEQ ID NO: 5001)
TGWVQD,
(SEQ ID NO: 4953)
RVEVQN,
(SEQ ID NO: 5073)
VLPVQN,
(SEQ ID NO: 4987)
TGGVQK,
(SEQ ID NO: 4964)
SVAVQN,
(SEQ ID NO: 4909)
LQQVQN,
(SEQ ID NO: 4917)
LSTVQN,
(SEQ ID NO: 4954)
SAAVQN,
(SEQ ID NO: 4951)
RTSVQN,
(SEQ ID NO: 5028)
TQNVQN,
(SEQ ID NO: 5078)
VNAVQN,
(SEQ ID NO: 5053)
TVAVQT,
(SEQ ID NO: 4906)
LMSVQN,
(SEQ ID NO: 4851)
TGWVQN,
(SEQ ID NO: 5105)
TGWVQH,
(SEQ ID NO: 4858)
TGWVQS,
(SEQ ID NO: 5205)
TMMVQN,
(SEQ ID NO: 5206)
TGGVQS,
(SEQ ID NO: 5207)
TGSFQN,
(SEQ ID NO: 5208)
RSVVQN,
(SEQ ID NO: 5209)
TGSVQK,
(SEQ ID NO: 5210)
TQLVQN,
(SEQ ID NO: 5211)
TGGVHN,
(SEQ ID NO: 5212)
RTPVQN,
(SEQ ID NO: 5213)
RQQVQN,
(SEQ ID NO: 5214)
TGSVRN,
(SEQ ID NO: 5215)
VQGVQN,
(SEQ ID NO: 5216)
PGWVQT,
(SEQ ID NO: 5217)
STQVQN,
(SEQ ID NO: 5218)
QSPVQN,
(SEQ ID NO: 5219)
RYSVQN,
(SEQ ID NO: 5220)
TQRVQN,
(SEQ ID NO: 5221)
SAGVQN,
(SEQ ID NO: 5222)
SAPVQT,
(SEQ ID NO: 5223)
RQSVQN,
(SEQ ID NO: 5224)
SQPVQN,
(SEQ ID NO: 5225)
VASVKN,
(SEQ ID NO: 5226)
TAWVRN,
(SEQ ID NO: 5227)
TGGGQN,
(SEQ ID NO: 5228)
STSVQN,
(SEQ ID NO: 5229)
VLGVQN,
(SEQ ID NO: 5230)
NQPVQN,
(SEQ ID NO: 5231)
LGTVQN,
(SEQ ID NO: 5232)
RAGVQN,
(SEQ ID NO: 5233)
TGGVKN,
(SEQ ID NO: 5234)
TAWLQN,
(SEQ ID NO: 5235)
TRWVQK,
(SEQ ID NO: 5236)
LAPVKN,
(SEQ ID NO: 5237)
TGSVQD,
(SEQ ID NO: 5238)
TGMVQN,
(SEQ ID NO: 5239)
LSNVQN,
(SEQ ID NO: 5240)
RLPVQN,
(SEQ ID NO: 5241)
RQGVQN,
(SEQ ID NO: 5242)
STPVQT,
(SEQ ID NO: 5243)
TTPVKN,
(SEQ ID NO: 5244)
RLTVQN,
(SEQ ID NO: 5245)
TLRVQN,
(SEQ ID NO: 5246)
SAFVQN,
(SEQ ID NO: 5247)
SVQVQN,
(SEQ ID NO: 5248)
LLPVQN,
(SEQ ID NO: 5249)
RTQVQN,
(SEQ ID NO: 5250)
TGSDQN,
(SEQ ID NO: 5251)
VASDQN,
(SEQ ID NO: 5252)
VSPVKN,
(SEQ ID NO: 5253)
LPPVQN,
(SEQ ID NO: 5254)
SSPVQT,
(SEQ ID NO: 5255)
AQPVQN,
(SEQ ID NO: 5256)
TPQVQN,
(SEQ ID NO: 5257)
TSWVQN,
(SEQ ID NO: 5258)
TGGDQN,
(SEQ ID NO: 5109)
LSPVKN,
(SEQ ID NO: 5259)
SSPVKN,
(SEQ ID NO: 5260)
NTTVQN,
(SEQ ID NO: 5261)
TTRVQN,
(SEQ ID NO: 5262)
TQWVQN,
(SEQ ID NO: 5263)
TGSVHN,
(SEQ ID NO: 5264)
TGGLQN,
(SEQ ID NO: 5265)
TAWVQK,
(SEQ ID NO: 5266)
TGRVQT,
(SEQ ID NO: 5267)
NTQVQN,
(SEQ ID NO: 5268)
TGWLQN,
(SEQ ID NO: 5269)
TYAVQN,
(SEQ ID NO: 5270)
TLSVQN,
(SEQ ID NO: 5271)
NLPVQN,
(SEQ ID NO: 5272)
TSSDQN,
(SEQ ID NO: 5273)
ATSVQN,
(SEQ ID NO: 5274)
TAWFQN,
(SEQ ID NO: 5275)
ATQVQN,
(SEQ ID NO: 5276)
VSSVKN,
(SEQ ID NO: 5107)
LSSVQN,
(SEQ ID NO: 5277)
TGSGQN,
(SEQ ID NO: 5278)
LQPVQT,
(SEQ ID NO: 5279)
VSAVKN,
(SEQ ID NO: 5280)
TQAVQN,
(SEQ ID NO: 5281)
TGWVQK,
(SEQ ID NO: 5282)
VMPVQN,
(SEQ ID NO: 5283)
TVSVQK,
(SEQ ID NO: 5284)
TAWAQN,
(SEQ ID NO: 5285)
NALVQN,
(SEQ ID NO: 5286)
RMLVQN,
(SEQ ID NO: 5287)
TVAVKN,
(SEQ ID NO: 5288)
RQLVQN,
(SEQ ID NO: 5289)
TLGVQN,
(SEQ ID NO: 5290)
LGPVQT,
(SEQ ID NO: 5291)
TGSVKN,
(SEQ ID NO: 5292)
TMSDQN,
(SEQ ID NO: 5293)
LASVKN,
(SEQ ID NO: 5294)
TGFVQN,
(SEQ ID NO: 5295)
SALVQN,
(SEQ ID NO: 5296)
TGWVKT,
(SEQ ID NO: 5297)
SQLVQN,
(SEQ ID NO: 5298)
TGWGQN,
(SEQ ID NO: 5299)
LSAVQN,
(SEQ ID NO: 5300)
TMQVQT,
(SEQ ID NO: 5301)
TGQVQN,
(SEQ ID NO: 5302)
TSPVKN,
(SEQ ID NO: 5303)
LSQVQT,
(SEQ ID NO: 5304)
TGSVLN,
(SEQ ID NO: 5305)
TNGVQN,
(SEQ ID NO: 5306)
TGWVKK,
(SEQ ID NO: 5307)
AAAVQN,
(SEQ ID NO: 5308)
SAVVQN,
(SEQ ID NO: 5309)
PGWVQH,
(SEQ ID NO: 5310)
TASDQN,
(SEQ ID NO: 5106)
LSGVQN,
(SEQ ID NO: 5311)
SSRVQN,
(SEQ ID NO: 5312)
SPPVQN,
(SEQ ID NO: 5313)
VQPVQT,
(SEQ ID NO: 5314)
TSSVKN,
(SEQ ID NO: 5315)
LSPLQN,
(SEQ ID NO: 5316)
VSQVQK,
(SEQ ID NO: 5317)
LVGVQN,
(SEQ ID NO: 5318)
TLSVKN,
(SEQ ID NO: 5319)
TGWFQK,
(SEQ ID NO: 5320)
TPAVQN,
(SEQ ID NO: 5321)
TVGVKN,
(SEQ ID NO: 5322)
KGWDQN,
(SEQ ID NO: 5323)
TAWVLN,
(SEQ ID NO: 5324)
VPPVQN,
(SEQ ID NO: 5325)
ATGVQN,
(SEQ ID NO: 5326)
TGGVQI,
(SEQ ID NO: 5327)
TGWVLN,
(SEQ ID NO: 5328)
TAWGQN,
(SEQ ID NO: 5329)
TGWVHN,
(SEQ ID NO: 5330)
LGSVQT,
(SEQ ID NO: 5331)
SANVQN,
(SEQ ID NO: 5332)
TGGVQD,
(SEQ ID NO: 5333)
TMAVKN,
(SEQ ID NO: 5334)
TASVKN,
(SEQ ID NO: 5335)
SSPVQK,
(SEQ ID NO: 5336)
TGTVQT,
(SEQ ID NO: 5337)
TGWVQI,
(SEQ ID NO: 5338)
TVWVKN,
(SEQ ID NO: 5339)
SQQVQN,
(SEQ ID NO: 5340)
VGSVQT,
(SEQ ID NO: 5341)
SSMVQN,
(SEQ ID NO: 5342)
TSPVQK,
(SEQ ID NO: 5343)
AVGVQN,
(SEQ ID NO: 5344)
VAPVQN,
(SEQ ID NO: 5345)
TLPVQK,
(SEQ ID NO: 5346)
TGRVQH,
(SEQ ID NO: 5347)
TPSVQN,
(SEQ ID NO: 5348)
TGWVEN,
(SEQ ID NO: 5349)
RGWVQN,
(SEQ ID NO: 5350)
TGSVEN,
(SEQ ID NO: 5351)
SSLVQN,
(SEQ ID NO: 5352)
TAWVKN,
(SEQ ID NO: 5353)
TYSVQN,
(SEQ ID NO: 5354)
LAAVQT,
(SEQ ID NO: 5355)
TALVKN,
(SEQ ID NO: 5356)
TGWVQY,
(SEQ ID NO: 5357)
TLPVQT,
(SEQ ID NO: 5358)
TGLVQH,
(SEQ ID NO: 5359)
TPTVQN,
(SEQ ID NO: 5360)
TASVQK,
(SEQ ID NO: 5361)
TSPVQI,
(SEQ ID NO: 5362)
IGWVQN,
(SEQ ID NO: 5363)
TGWDKN,
(SEQ ID NO: 5364)
KSSVQN,
(SEQ ID NO: 5365)
TGYVQN,
(SEQ ID NO: 5366)
RGWVQT,
(SEQ ID NO: 5367)
RSLVQN,
(SEQ ID NO: 5368)
TGGVEN,
(SEQ ID NO: 5369)
TGCVRN,
(SEQ ID NO: 5370)
LSPVQS,
(SEQ ID NO: 5371)
TGPVQT,
(SEQ ID NO: 5372)
TVGVQK,
(SEQ ID NO: 5373)
TASGQN,
(SEQ ID NO: 5374)
SVSVQN,
(SEQ ID NO: 5375)
SGPVQT,
(SEQ ID NO: 5376)
VMSVKN,
(SEQ ID NO: 5377)
LGSVQK,
(SEQ ID NO: 5378)
TGLVLN,
(SEQ ID NO: 5379)
TSNVQN,
(SEQ ID NO: 5380)
TGWGHN,
(SEQ ID NO: 5381)
SQMVQN,
(SEQ ID NO: 5382)
TVSVHN,
(SEQ ID NO: 5383)
LSSVQT,
(SEQ ID NO: 5384)
TASVRN,
(SEQ ID NO: 5385)
VPAVQN,
(SEQ ID NO: 5386)
TGRVQK,
(SEQ ID NO: 5387)
AMSVQN,
(SEQ ID NO: 5388)
TAWVHN,
(SEQ ID NO: 5389)
TGLVRN,
(SEQ ID NO: 5390)
RTLVQT,
(SEQ ID NO: 5391)
TGSIQN,
(SEQ ID NO: 5392)
LSSVKN,
(SEQ ID NO: 5393)
TLQVQK,
(SEQ ID NO: 5394)
VGSVKN,
(SEQ ID NO: 5395)
LAPLQN,
(SEQ ID NO: 5396)
TPGVQN,
(SEQ ID NO: 5397)
LSAVQT,
(SEQ ID NO: 5398)
TGVVQN,
(SEQ ID NO: 5399)
VPQVQN,
(SEQ ID NO: 5400)
TGCVQK,
(SEQ ID NO: 5401)
TRWVQT,
(SEQ ID NO: 5402)
TGLDON,
(SEQ ID NO: 5403)
VSSVHN,
(SEQ ID NO: 5404)
KGWVQT,
(SEQ ID NO: 5405)
SLPVQN,
(SEQ ID NO: 5406)
TTSVHN,
(SEQ ID NO: 5407)
TVWVQN,
(SEQ ID NO: 5408)
TAQLQN,
(SEQ ID NO: 5409)
TRWVKN,
(SEQ ID NO: 5410)
TAWIQN,
(SEQ ID NO: 5411)
LSQVKN,
(SEQ ID NO: 5412)
TSTVKN,
(SEQ ID NO: 5413)
ALPVQN,
(SEQ ID NO: 5414)
TSMVQT,
(SEQ ID NO: 5415)
TSSVQH,
(SEQ ID NO: 5416)
TAMVKN,
(SEQ ID NO: 5417)
TPWVQN,
(SEQ ID NO: 5418)
TPRVQN,
(SEQ ID NO: 5419)
SSSVQN,
(SEQ ID NO: 5420)
RPPVQN,
(SEQ ID NO: 5421)
LAGVKN,
(SEQ ID NO: 5422)
TSPAQN,
(SEQ ID NO: 5423)
RSPVQT,
(SEQ ID NO: 5424)
TGWVPH,
(SEQ ID NO: 5425)
PGWGQN,
(SEQ ID NO: 5426)
IPPVQN,
(SEQ ID NO: 5427)
TGRVKN,
(SEQ ID NO: 5428)
TGRLQN,
(SEQ ID NO: 5429)
LSSVQH,
(SEQ ID NO: 5430)
AGWVQT,
(SEQ ID NO: 5431)
TGLVQS,
(SEQ ID NO: 5432)
TGCVQI,
(SEQ ID NO: 5433)
RPGVQN,
(SEQ ID NO: 5434)
TAAVQH,
(SEQ ID NO: 5435)
TGCDQN,
(SEQ ID NO: 5436)
TGRVRN,
(SEQ ID NO: 5437)
TGRDQN;
(ii)
(SEQ ID NO: 5047)
TTKVQN,
(SEQ ID NO: 5013)
TMKVQN,
(SEQ ID NO: 5062)
VAQVQN,
(SEQ ID NO: 4978)
TAWDQN,
(SEQ ID NO: 4992)
TGSVQH,
(SEQ ID NO: 5072)
VKQVQN,
(SEQ ID NO: 4854)
SAPVQN,
(SEQ ID NO: 4912)
LSKVQN,
(SEQ ID NO: 4891)
LAPVQN,
(SEQ ID NO: 4893)
LAQVQN,
(SEQ ID NO: 4968)
TAKVQN,
(SEQ ID NO: 4955)
SAKVQN,
(SEQ ID NO: 4982)
TGCFQN,
(SEQ ID NO: 5026)
TQKVQN,
(SEQ ID NO: 5052)
TVAVQN,
(SEQ ID NO: 4914)
LSPVQN,
(SEQ ID NO: 5050)
TTQVQN,
(SEQ ID NO: 4973)
TAQVQN,
(SEQ ID NO: 4934)
RIAVQN,
(SEQ ID NO: 4931)
RASVQN,
(SEQ ID NO: 5048)
TTPVQN,
(SEQ ID NO: 4853)
LTPVQN,
(SEQ ID NO: 4963)
STPVQN,
(SEQ ID NO: 4861)
TSPVQN,
(SEQ ID NO: 5016)
TMQVQN,
(SEQ ID NO: 5036)
TSKVQN,
(SEQ ID NO: 5092)
VSQVQN,
(SEQ ID NO: 4868)
VSPVQN,
(SEQ ID NO: 5055)
TVQVQN,
(SEQ ID NO: 5097)
VTAVQN,
(SEQ ID NO: 4941)
RQPVQN,
(SEQ ID NO: 4884)
ISGVQN,
(SEQ ID NO: 5087)
VRPVQN,
(SEQ ID NO: 4897)
LGPVQN,
(SEQ ID NO: 5022)
TNQVQN,
(SEQ ID NO: 5084)
VQQVQN,
(SEQ ID NO: 5061)
VANVQN,
(SEQ ID NO: 4870)
AAPVQN,
(SEQ ID NO: 4947)
RSTVQN,
(SEQ ID NO: 5011)
TMAVQN,
(SEQ ID NO: 4882)
IQPVQN,
(SEQ ID NO: 4878)
IASVQN,
(SEQ ID NO: 4867)
TVSVQN,
(SEQ ID NO: 4933)
RGSVQN,
(SEQ ID NO: 4925)
NSPVQN,
(SEQ ID NO: 4908)
LQPVQN,
(SEQ ID NO: 5098)
VTGVQN,
(SEQ ID NO: 5076)
VMQVQN,
(SEQ ID NO: 4959)
SMAVQN,
(SEQ ID NO: 5066)
VGKVQN,
(SEQ ID NO: 4883)
IQSVQN,
(SEQ ID NO: 4874)
CSPVQN,
(SEQ ID NO: 4910)
LQRVQN,
(SEQ ID NO: 4979)
TAWVQH,
(SEQ ID NO: 5024)
TPPVQN,
(SEQ ID NO: 5099)
VTKVQN,
(SEQ ID NO: 4855)
SSPVQN,
(SEQ ID NO: 4871)
AGPVQN,
(SEQ ID NO: 4894)
LARVQN,
(SEQ ID NO: 5051)
TTTVQN,
(SEQ ID NO: 4984)
TGGFQN,
(SEQ ID NO: 5010)
TLQVQN,
(SEQ ID NO: 5018)
TMSVQN,
(SEQ ID NO: 5060)
VAKVQN,
(SEQ ID NO: 4929)
RAAVQN,
(SEQ ID NO: 5054)
TVGVQN,
(SEQ ID NO: 4907)
LNPVQN,
(SEQ ID NO: 4915)
LSQVQN,
(SEQ ID NO: 5006)
TKPVQN,
(SEQ ID NO: 5021)
TNAVQN,
(SEQ ID NO: 4896)
LATVQN,
(SEQ ID NO: 5100)
VTPVQN,
(SEQ ID NO: 5081)
VQAVQN,
(SEQ ID NO: 4860)
TTSVQN,
(SEQ ID NO: 4875)
CTPVQN,
(SEQ ID NO: 4967)
TAGVQN,
(SEQ ID NO: 5040)
TSQVQN,
(SEQ ID NO: 5014)
TMNVQN,
(SEQ ID NO: 5043)
TSTVQN,
(SEQ ID NO: 5071)
VKPVQN,
(SEQ ID NO: 4872)
ASPVQN,
(SEQ ID NO: 5058)
VAAVQN,
(SEQ ID NO: 4901)
LKSVQN,
(SEQ ID NO: 4876)
IAAVQN,
(SEQ ID NO: 4966)
TAAVQN,
(SEQ ID NO: 5005)
TKAVQN,
(SEQ ID NO: 4994)
TGSVQS,
(SEQ ID NO: 5090)
VSNVQN,
(SEQ ID NO: 4971)
TAPVQN,
(SEQ ID NO: 4904)
LMPVQN,
(SEQ ID NO: 4900)
LHPVQN,
(SEQ ID NO: 4930)
RAQVQN,
(SEQ ID NO: 4919)
LTNVQN,
(SEQ ID NO: 4952)
RTTVQN,
(SEQ ID NO: 5044)
TSVVQN,
(SEQ ID NO: 4939)
RMSVQN,
(SEQ ID NO: 5067)
VGNVQN,
(SEQ ID NO: 4905)
LMQVQN,
(SEQ ID NO: 4976)
TATVQN,
(SEQ ID NO: 5069)
VHPVQN,
(SEQ ID NO: 5091)
VSPVQT,
(SEQ ID NO: 4886)
ISSVQN,
(SEQ ID NO: 5063)
VASVQN,
(SEQ ID NO: 5032)
TRWDQN,
(SEQ ID NO: 5020)
TMTVQN,
(SEQ ID NO: 4946)
RSSVQN,
(SEQ ID NO: 4863)
TAWVQN,
(SEQ ID NO: 4949)
RTGVQN,
(SEQ ID NO: 5064)
VATVQN,
(SEQ ID NO: 5101)
VTSVQN,
(SEQ ID NO: 4869)
VSSVQN,
(SEQ ID NO: 5023)
TNSVQN,
(SEQ ID NO: 5070)
VKAVQN,
(SEQ ID NO: 4958)
SGPVQN,
(SEQ ID NO: 4989)
TGPVQN,
(SEQ ID NO: 4969)
TAMVQN,
(SEQ ID NO: 5029)
TQPVQN,
(SEQ ID NO: 5030)
TQQVQN,
(SEQ ID NO: 5093)
VSRVQN,
(SEQ ID NO: 5002)
TGWVQP,
(SEQ ID NO: 5088)
VSAVQN,
(SEQ ID NO: 5074)
VLSVQN,
(SEQ ID NO: 5025)
TQHVQN,
(SEQ ID NO: 4895)
LASVQN,
(SEQ ID NO: 4928)
QAPVQN,
(SEQ ID NO: 4922)
NAQVQN,
(SEQ ID NO: 4873)
ATPVQN,
(SEQ ID NO: 5083)
VQPVQN,
(SEQ ID NO: 5045)
TTAVQN,
(SEQ ID NO: 5004)
TGWVRN,
(SEQ ID NO: 4852)
LAAVQN,
(SEQ ID NO: 5039)
TSPDQN,
(SEQ ID NO: 4943)
RSGVQN,
(SEQ ID NO: 4988)
TGGVQT,
(SEQ ID NO: 5000)
TGWVPN,
(SEQ ID NO: 5049)
TTPVQT,
(SEQ ID NO: 4902)
LMAVQN,
(SEQ ID NO: 5015)
TMPVQN,
(SEQ ID NO: 4890)
LANVQN,
(SEQ ID NO: 5096)
VSTVQN,
(SEQ ID NO: 4957)
SAQVQN,
(SEQ ID NO: 4926)
NTPVQN,
(SEQ ID NO: 5095)
VSSVQT,
(SEQ ID NO: 5056)
TVSVKN,
(SEQ ID NO: 5102)
TGLVQN,
(SEQ ID NO: 4865)
TGSVQN,
(SEQ ID NO: 4983)
TGGAQN,
(SEQ ID NO: 4977)
TAVVQN,
(SEQ ID NO: 4937)
RLGVQN,
(SEQ ID NO: 4950)
RTLVQN,
(SEQ ID NO: 5027)
TQMVQN,
(SEQ ID NO: 4887)
ITPVQN,
(SEQ ID NO: 5057)
TVWVQK,
(SEQ ID NO: 4942)
RSAVQN,
(SEQ ID NO: 4974)
TASVQN,
(SEQ ID NO: 5012)
TMGVQN,
(SEQ ID NO: 4986)
TGGVQH,
(SEQ ID NO: 5085)
VQSVQN,
(SEQ ID NO: 4864)
TGGVQN,
(SEQ ID NO: 4885)
ISPVQN,
(SEQ ID NO: 5103)
TGWVKN,
(SEQ ID NO: 4990)
TGSAQN,
(SEQ ID NO: 4857)
TGWAQN,
(SEQ ID NO: 5019)
TMSVQT,
(SEQ ID NO: 5065)
VGGVQN,
(SEQ ID NO: 4892)
LAPVQT,
(SEQ ID NO: 4862)
TALVQN,
(SEQ ID NO: 4888)
LAGVQN,
(SEQ ID NO: 4948)
RTAVQN,
(SEQ ID NO: 4944)
RSPVQN,
(SEQ ID NO: 4859)
TLAVQN,
(SEQ ID NO: 4889)
LAHVQN,
(SEQ ID NO: 5037)
TSLVQN,
(SEQ ID NO: 4938)
RLSVQN,
(SEQ ID NO: 4903)
LMGVQN,
(SEQ ID NO: 4961)
SMQVQN,
(SEQ ID NO: 5031)
TQTVQN,
(SEQ ID NO: 4997)
TGWEQN,
(SEQ ID NO: 5068)
VGSVQN,
(SEQ ID NO: 5089)
VSGVQN,
(SEQ ID NO: 5075)
VMAVQN,
(SEQ ID NO: 4879)
IGGVQN,
(SEQ ID NO: 4877)
IAGVQN,
(SEQ ID NO: 4856)
TGRVQN,
(SEQ ID NO: 4911)
LSHVQN,
(SEQ ID NO: 5086)
VQTVQN,
(SEQ ID NO: 4866)
TGWDQN,
(SEQ ID NO: 4940)
RNSVQN,
(SEQ ID NO: 5009)
TLPVQN,
(SEQ ID NO: 5007)
TKQVQN,
(SEQ ID NO: 4898)
LGQVQN,
(SEQ ID NO: 4924)
NMQVQN,
(SEQ ID NO: 4927)
NVQVQN,
(SEQ ID NO: 4993)
TGSVQI,
(SEQ ID NO: 4932)
RGGVQN,
(SEQ ID NO: 5077)
VMSVQN,
(SEQ ID NO: 5046)
TTGVQN,
(SEQ ID NO: 4913)
LSPVQK,
(SEQ ID NO: 4916)
LSRVQN,
(SEQ ID NO: 5094)
VSSVQK,
(SEQ ID NO: 4972)
TAPVQT,
(SEQ ID NO: 4921)
MAPVQN,
(SEQ ID NO: 4881)
ILGVQN,
(SEQ ID NO: 4975)
TASVQT,
(SEQ ID NO: 4991)
TGSLON,
(SEQ ID NO: 4996)
TGTVQN,
(SEQ ID NO: 4995)
TGSVQT,
(SEQ ID NO: 5042)
TSSVQT,
(SEQ ID NO: 5035)
TSHVQN,
(SEQ ID NO: 4935)
RIGVQN,
(SEQ ID NO: 4999)
TGWGQT,
(SEQ ID NO: 4956)
SAMVQN,
(SEQ ID NO: 5038)
TSMVQN,
(SEQ ID NO: 4960)
SMGVQN,
(SEQ ID NO: 4962)
SMSVQN,
(SEQ ID NO: 5041)
TSSVQN,
(SEQ ID NO: 5034)
TSGVQN,
(SEQ ID NO: 4981)
TGAVQN,
(SEQ ID NO: 5080)
VNSVQN,
(SEQ ID NO: 5059)
VAGVQN,
(SEQ ID NO: 4880)
IGSVQN,
(SEQ ID NO: 4899)
LGSVQN,
(SEQ ID NO: 5079)
VNGVQN,
(SEQ ID NO: 4918)
LTAVQN,
(SEQ ID NO: 5082)
VQNVQN,
(SEQ ID NO: 5008)
TKSVQN,
(SEQ ID NO: 4965)
SVGVQN,
(SEQ ID NO: 4980)
TAWVQT,
(SEQ ID NO: 4923)
NASVQN,
(SEQ ID NO: 5033)
TSAVQN,
(SEQ ID NO: 5017)
TMSVKN,
(SEQ ID NO: 4970)
TANVQN,
(SEQ ID NO: 4998)
TGWFQN,
(SEQ ID NO: 4985)
TGGVLN,
(SEQ ID NO: 4920)
LTSVQN,
(SEQ ID NO: 5003)
TGWVQT,
(SEQ ID NO: 4945)
RSQVQN,
(SEQ ID NO: 4936)
RIPVQN,
(SEQ ID NO: 5001)
TGWVQD,
(SEQ ID NO: 4953)
RVEVQN,
(SEQ ID NO: 5073)
VLPVQN,
(SEQ ID NO: 4987)
TGGVQK,
(SEQ ID NO: 4964)
SVAVQN,
(SEQ ID NO: 4909)
LQQVQN,
(SEQ ID NO: 4917)
LSTVQN,
(SEQ ID NO: 4954)
SAAVQN,
(SEQ ID NO: 4951)
RTSVQN,
(SEQ ID NO: 5028)
TQNVQN,
(SEQ ID NO: 5078)
VNAVQN,
(SEQ ID NO: 5053)
TVAVQT,
(SEQ ID NO: 4906)
LMSVQN,
(SEQ ID NO: 4851)
TGWVQN;

(iii) amino acid sequence comprising any portion of an amino acid sequence in (i) or (ii), e.g., any 2, 3, 4, or 5 amino acids, e.g., consecutive amino acids, thereof;
(iv) an amino acid sequence comprising at least one, two, or three but no more than four modifications, e.g., substitutions (e.g., conservative substitutions), insertions, or deletions, relative to any of the amino acid sequences in (i) or (ii); or
(v) an amino acid sequence comprising at least one, two, or three but no more than four different amino acids, relative to any one of the amino acid sequences in (i) or (ii).
182. The isolated AAV particle of any one of embodiments 171-181, wherein [D][E] is or comprises TGWVQN (SEQ ID NO: 4851), TGWVPN (SEQ ID NO: 5000), or LSPVKN (SEQ ID NO: 5109).
183. The isolated AAV particle of any one of embodiments 171-182, wherein:
   (i) [B] is or comprises: VHLY (SEQ ID NO: 4680), VHHY (SEQ ID NO: 4683), VHIY (SEQ ID NO: 4681), VNLY (SEQ ID NO: 4724), VQLY (SEQ ID NO: 4729), IHLY (SEQ ID NO: 4730), LHLY (SEQ ID NO: 4727), VPLY (SEQ ID NO: 4723), VDLY (SEQ ID NO: 4731), AHLY (SEQ ID NO: 4732), VHRY (SEQ ID NO: 4725), FHLY (SEQ ID NO: 4726), DHLY (SEQ ID NO: 4728), VLLY (SEQ ID NO: 4733), GHLY (SEQ ID NO: 4734), VRLY (SEQ ID NO: 4735), VHVY (SEQ ID NO: 4682), or VYLY (SEQ ID NO: 4736);
   (ii) [C] is or comprises: AQAQ (SEQ ID NO: 4737), AQPQ (SEQ ID NO: 4739), AKAQ (SEQ ID NO: 4741), DQAQ (SEQ ID NO: 4744), SQAQ (SEQ ID NO: 4738), AHAQ (SEQ ID NO: 4742), AQEQ (SEQ ID NO: 4748), AQAK (SEQ ID NO: 4746), ALAQ (SEQ ID NO: 4749), APAQ (SEQ ID NO: 4745), ARAQ (SEQ ID NO: 4750), AQAH (SEQ ID NO: 4747), AQAP (SEQ ID NO: 4743), or TQAQ (SEQ ID NO: 4751);
   (iii) [D] is or comprises: TTK, TMK, VAQ, TAW, TGS, VKQ, SAP, LSK, LAP, LAQ, TAK, SAK, TGC, TQK, TVA, LSP, TTQ, TAQ, RIA, RAS, TTP, LTP, STP, TSP, TMQ, TSK, VSQ, VSP, TVQ, VTA, RQP, ISG, VRP, LGP, TNQ, VQQ, VAN, AAP, RST, TMA, IQP, IAS, TVS, RGS, NSP, LQP, VTG, VMQ, SMA, VGK, IQS, CSP, LQR, TPP, VTK, SSP, AGP, LAR, TTT, TGG, TLQ, TMS, VAK, RAA, TVG, LNP, LSQ, TKP, TNA, LAT, VTP, VQA, TTS, CTP, TAG, TSQ, TMN, TST, VKP, ASP, VAA, LKS, IAA, TAA, TKA, VSN, TAP, LMP, LHP, RAQ, LTN, RTT, TSV, RMS, VGN, LMQ, TAT, VHP, ISS, VAS, TRW, TMT, RSS, RTG, VAT, VTS, VSS, TNS, VKA, SGP, TGP, TAM, TQP, TQQ, VSR, TGW, VSA, VLS, TQH, LAS, QAP, NAQ, ATP, VQP, TTA, LAA, RSG, LMA, TMP, LAN, VST, SAQ, NTP, TGL, TAV, RLG, RTL, TQM, ITP, TVW, RSA, TAS, TMG, VQS, ISP, VGG, TAL, LAG, RTA, RSP, TLA, LAH, TSL, RLS, LMG, SMQ, TQT, VGS, VSG, VMA, IGG, IAG, TGR, LSH, VQT, RNS, TLP, TKQ, LGQ, NMQ, NVQ, RGG, VMS, TTG, LSR, MAP, ILG, TGT, TSS, TSH, RIG, SAM, TSM, SMG, SMS, TSG, TGA, VNS, VAG, IGS, LGS, VNG, LTA, VQN, TKS, SVG, NAS, TSA, TAN, LTS, RSQ, RIP, RVE, VLP, SVA, LQQ, LST, SAA, RTS, TQN, VNA, LMS, TMM, RSV, TQL, RTP, RQQ, VQG, PGW, STQ, QSP, RYS, TQR, SAG, RQS, SQP, STS, VLG, NQP, LGT, RAG, TGM, LSN, RLP, RQG, RLT, TLR, SAF, SVQ, LLP, RTQ, LPP, AQP, TPQ, TSW, NTT, TTR, TQW, NTQ, TYA, TLS, NLP, ATS, ATQ, LSS, TQA, VMP, NAL, RML, RQL, TLG, TGF, SAL, SQL, LSA, TGQ, TNG, AAA, SAV, LSG, SSR, SPP, LVG, TPA, KGW, VPP, ATG, SAN, SQQ, SSM, AVG, VAP, TPS, RGW, SSL, TYS, TPT, IGW, KSS, TGY, RSL, SVS, TSN, SQM, VPA, AMS, TPG, TGV, VPQ, SLP, ALP, TPW, TPR, SSS, RPP, IPP, AGW, or RPG; and/or
   (iv) [E] is or comprises: VQN, DQN, VQH, FQN, VQS, VQT, VQP, VRN, VPN, VKN, AQN, VQK, EQN, VQI, LQN, GQT, VLN, VQD, VHN, GQN, VKT, VKK, FQK, VEN, VQY, DKN, GHN, IQN, or VPH.
184. The isolated AAV particle of any one of embodiments 171-183, wherein:
   (i) [B] is or comprises: VHLY (SEQ ID NO: 4680), VHHY (SEQ ID NO: 4683), or VHIY (SEQ ID NO: 4681);
   (ii) [C] is or comprises: AQAQ (SEQ ID NO: 4737), AQPQ (SEQ ID NO: 4739), AKAQ (SEQ ID NO: 4741), DQAQ (SEQ ID NO: 4744), or SQAQ (SEQ ID NO: 4738);
   (iii) [D] is or comprises: TTK, TMK, VAQ, TAW, TGS, VKQ, SAP, LSK, LAP, LAQ, TAK, SAK, TGC, TQK, TVA, LSP, TTQ, TAQ, RIA, RAS, TTP, LTP, STP, TSP, TMQ, TSK, VSQ, VSP, TVQ, VTA, RQP, ISG, VRP, LGP, TNQ, VQQ, VAN, AAP, RST, TMA, IQP, IAS, TVS, RGS, NSP, LQP, VTG, VMQ, SMA, VGK, IQS, CSP, LQR, TPP, VTK, SSP, AGP, LAR, TTT, TGG, TLQ, TMS, VAK, RAA, TVG, LNP, LSQ, TKP, TNA, LAT, VTP, VQA, TTS, CTP, TAG, TSQ, TMN, TST, VKP, ASP, VAA, LKS, IAA, TAA, TKA, VSN, TAP, LMP, LHP, RAQ, LTN, RTT, TSV, RMS, VGN, LMQ, TAT, VHP, ISS, VAS, TRW, TMT, RSS, RTG, VAT, VTS, VSS, TNS, VKA, SGP, TGP, TAM, TQP, TQQ, VSR, TGW, VSA, VLS, TQH, LAS, QAP, NAQ, ATP, VQP, TTA, LAA, RSG, LMA, TMP, LAN, VST, SAQ, NTP, TGL, TAV, RLG, RTL, TQM, ITP, TVW, RSA, TAS, TMG, VQS, ISP, VGG, TAL, LAG, RTA, RSP, TLA, LAH, TSL, RLS, LMG, SMQ, TQT, VGS, VSG, VMA, IGG, IAG, TGR, LSH, VQT, RNS, TLP, TKQ, LGQ, NMQ, NVQ, RGG, VMS, TTG, LSR, MAP, ILG, TGT, TSS, TSH, RIG, SAM, TSM, SMG, SMS, TSG, TGA, VNS, VAG, IGS, LGS, VNG, LTA, VQN, TKS, SVG, NAS, TSA, TAN, LTS, RSQ, RIP, RVE, VLP, SVA, LQQ, LST, SAA, RTS, TQN, VNA, or LMS; and/or
   (iv) [E] is or comprises: VQN, DQN, VQH, FQN, VQS, VQT, VQP, VRN, VPN, VKN, AQN, VQK, EQN, VQI, LQN, GQT, VLN, or VQD.
185. The isolated AAV particle of any one of embodiments 171-184, wherein [A][B][C][D][E] comprises:
   (i) the amino acid sequence of any of SEQ ID NOs: 143, 148, 149, 151, 153, 154-158, 160-163, 166, 168, 170, 171, 173-175, 177-179, 181, 182, 184-188, 191-197, 199-210, 212-215, 217-225, 227-231, 233, 234, 236-240, 243-262, 265, 267, 268, 270-277, 279, 282, 284-286, 288-293, 295, 296, 298, 300-314, 316-327, 329, 331, 332, 334, 336, 337-344, 346-350, 352-354, 356-365, 367, 369, 371-380, 382-385, 387, 392-394, 396, 397, 399-401, 404-411, 413-415, 417, 419-429, 432, 433, 435-437, 438, 440-442, 444-447, 450-454, 456, 458-461, 464, 465, 467-469, 471-484, 487-495, 497, 498, 500-503, 505, 507-512, 514-517, 522-525, 528-539, 542-545, 547, 551-555, 558-561, 563-568, 570, 573, 574, 576, 579, 581, 582, 584, 586, 587, 591-596, 598, 601, 604, 605, 606, 607, 610, 612, 614-619, 624-629, 631-636, 640, 641, 645, 646, 649, 650, 656, 658, 661, 663, 664, 666, 668, 669, 670, 672, 673, 674, 675, 677, 679, 683, 684, 686, 688, 689, 691, 693, 695, 696, 697, 699, 700, 701, 702, 704-706, 709-714, 720, 722, 725-731, 733, 736, 740, 745, 749-752, 754, 755, 757, 758, 760-765, 767, 768, 770, 771, 773, 778-780, 783-788, 792-794, 797-799, 801, 802, 804-806, 812, 814, 815, 817, 818, 820, 821, 824, 828, 831, 832, 834-837, 839, 840-845, 847, 848, 850-855, 857-859, 861, 862, 865, 866, 869-872, 874-876, 882-884, 887, 889-895, 897, 899, 901, 903-905, 907, 908, 910, 911, 913, 915, 919, 920, 923, 924, 926, 927, 929, 931-933, 935, 937, 939-949, 952-955, 957, 958, 960, 962, 964, 965, 967, 971, 973, 974, 976, 977, 981, 985-989, 992, 994, 997-1000, 1002, 1004, 1006-1008, 1010, 1013, 1015, 1017, 1018, 1020, 1021, 1023-1025, 1027, 1029-1031, 1033-1035, 1037-1040, 1043, 1046, 1049, 1052, 1053, 1056, 1057, 1059, 1062, 1064, 1065, 1067, 1068, 1070, 1073, 1075, 1077-1080, 1083-1087, 1089, 1090, 1093, 1094, 1097, 1100, 1101, 1103, 1105-1107, 1110-1112, 1114-1117, 1119, 1121, 1125, 1126, 1129, 1132, 1133, 1135;
   (ii) an amino acid sequence comprising any portion of an amino acid sequence in (i), e.g., any 2, 3, 4, 5, 6, 7, 8, 9, 10, 11, 12, 13, 14, 15, 16, 17, or 18 amino acids, e.g., consecutive amino acids, thereof;
   (iii) an amino acid sequence comprising at least one, two, or three but no more than four modifications, e.g., substitutions (e.g., conservative substitutions), insertions, or deletions, relative to any of the amino acid sequences in (i); or
   (iv) an amino acid sequence comprising at least one, two, or three but no more than four different amino acids, relative to any one of the amino acid sequences in (i).
186. The isolated AAV particle of any one of embodiments 171-185, wherein [A][B][C][D][E] comprises:
   (i) the amino acid sequence of any of SEQ ID NOs: 139, 143, 148, 149, 151, 153-158, 160-163, 166, 168, 170, 171, 173-175, 177-179, 181, 182, 184-188, 191-197, 199, 200, 201-210, 212-215, 217-225, 227-231, 233, 234, 236-240, 243-262, 265, 267, 268, 270-277, 279, 282, 284-286, 288-293, 295, 296, 298, 300-314, 316-327, 329, 331, 332, 334, 336, 337-344, 346-350, 352-354, 356-365, 367, 369, 371-380, 382-385, 387, 392-394, 396, 397, 399-401, 404, 405, 406-411, 413-415, 417, 419-429, 432, 433, 435-438, 440-442, 444-447, 450-454, 456, 458-461, 464, 465, 467-469, or 471-476;
   (ii) an amino acid sequence comprising any portion of an amino acid sequence in (i), e.g., any 2, 3, 4, 5, 6, 7, 8, 9, 10, 11, 12, 13, 14, 15, 16, 17, or 18 amino acids, e.g., consecutive amino acids, thereof;
   (iii) an amino acid sequence comprising at least one, two, or three but no more than four modifications, e.g., substitutions (e.g., conservative substitutions), insertions, or deletions, relative to any of the amino acid sequences in (i); or
   (iv) an amino acid sequence comprising at least one, two, or three but no more than four different amino acids, relative to any one of the amino acid sequences in (i).
187. The isolated AAV particle of any one of embodiments 171-186, wherein [A][B][C][D][E] comprises the amino acid sequence of PLNGAVHLYAQAQTGWVPN (SEQ ID NO: 314).
188. The isolated AAV particle of any one of embodiments 171-186, wherein [A][B][C][D][E] comprises the amino acid sequence of PLNGAVHLYAQAQLSPVKN (SEQ ID NO: 566).
189. The isolated AAV particle of any one of embodiments 171-186, wherein [A][B][C][D][E] comprises the amino acid sequence the amino acid sequence of PLNGAVHLYAQAQTGWVQN (SEQ ID NO: 476).
190. The isolated AAV particle of any one of embodiments 132-189, wherein [A][B] is present in loop VIII.
191. The isolated AAV particle of any one of embodiments 171-190, wherein [C], [D], and/or [E] is present in loop VIII.
192. The isolated AAV particle of any one of embodiments 171-191, wherein [A][B][C][D][E] is present in loop VIII.
193. The isolated AAV particle of any one of embodiments 171-192, which comprises an amino acid other than A at position 587 and/or an amino acid other than Q at position 588, numbered according to SEQ ID NO: 138.
194. The isolated AAV particle of any one of embodiments 171-193, which comprises:
   (i) the amino acid P at position 587, numbered according to SEQ ID NO: 5, 8, 138, or 3636; and/or
   (ii) the amino acid L at position 588, numbered according to SEQ ID NO: 5, 8, 138, or 3636.
195. The isolated AAV particle of any one of embodiments 132-194, wherein [A] is present immediately subsequent to position 586, relative to a reference sequence numbered according to the amino acid sequence of SEQ ID NO: 5, 8, 138, or 3636.
196. The isolated AAV particle of any one of embodiments 132-195, wherein [A] is present immediately subsequent to position 586, and replaces positions 587 and 588 (e.g., A587 and Q588), numbered according to SEQ ID NO: 138.
197. The isolated AAV particle of any one of embodiments 132-196, wherein [A] replaces positions 587 and 588 (e.g., A587 and Q588), numbered according to SEQ ID NO: 138.
198. The isolated AAV particle of any one of embodiments 132-197, wherein [A] corresponds to positions 587-591 of SEQ ID NO: 5, 8, or 3636.
199. The isolated AAV particle of any one of embodiments 132-198, wherein [B] is present immediately subsequent to [A].
200. The isolated AAV particle of any one of embodiments 132-199, wherein [B] is present immediately subsequent to [A], wherein [A] is present immediately subsequent to position 586, relative to a reference sequence numbered according to the amino acid sequence of SEQ ID NO: 5, 8, 138, or 3636.
201. The isolated AAV particle of any one of embodiments 132-200, wherein [B] is present immediately subsequent to [A], wherein [A] is present immediately subsequent to position 586 and replaces positions 587 and 588 (e.g., A587 and Q588), numbered according to SEQ ID NO: 138.
202. The isolated AAV particle of any one of embodiments 132-201, wherein [B] corresponds to positions 592 to 595 of SEQ ID NO: 5, 8, or 3636.
203. The isolated AAV particle of any one of embodiments 132-202, wherein [A][B] replaces positions 587 and 588 (e.g., A587 and Q588), numbered according to SEQ ID NO: 138.
204. The isolated AAV particle of any one of embodiments 132-203, wherein [A][B] is present immediately subsequent to position 586, numbered according to SEQ ID NO: 138.
205. The isolated AAV particle of any one of embodiments 132-204, wherein [A][B] is present immediately subsequent to position 586 and replaces positions 587 and 588 (e.g., A587 and Q588), numbered according to SEQ ID NO: 138.
206. The isolated AAV particle of any one of embodiments 132-205, wherein [A][B] corresponds to positions 587-595 of SEQ ID NO: 5, 8, or 3636.
207. The isolated AAV particle of any one of embodiments 144-206, wherein [C] is present immediately subsequent to position 588, relative to a reference sequence numbered according to the amino acid sequence of SEQ ID NO: 138.
208. The isolated AAV particle of any one of embodiments 144-207, wherein [C] replaces positions 589-592 (e.g., A589, Q590, A591, Q592), numbered according to the amino acid sequence of SEQ ID NO: 138.
209. The isolated AAV particle of any one of embodiments 144-208, wherein [C] is present immediately subsequent to position 588, and replaces positions 589-592 (e.g., A589, Q590, A591, Q592), numbered according to the amino acid sequence of SEQ ID NO: 138.
210. The isolated AAV particle of any one of embodiments 144-209, wherein [C] corresponds to positions 596-599 of SEQ ID NO: 5, 8, or 3636.
211. The isolated AAV particle of any one of embodiments 144-210, wherein [A][B][C] is present immediately subsequent to position 586, numbered according to SEQ ID NO: 138.
212. The isolated AAV particle of any one of embodiments 144-211, wherein [A][B][C] replaces positions 587-592 (e.g., A587, Q588, A589, Q590, A591, Q592), numbered according to SEQ ID NO: 138.
213. The isolated AAV particle of any one of embodiments 144-212, wherein [A][B][C] is present immediately subsequent to position 586 and replaces positions 587-592 (e.g., A587, Q588, A589, Q590, A591, Q592), numbered according to SEQ ID NO: 138.
214. The isolated AAV particle of any one of embodiments 144-213, wherein [A][B][C] corresponds to positions 587-599 of SEQ ID NO: 5, 8, or 3636.
215. The isolated AAV particle of any one of embodiments 160-214, wherein [D] is present immediately subsequent to position 592, relative to a reference sequence numbered according to the amino acid sequence of SEQ ID NO: 138.
216. The isolated AAV particle of any one of embodiments 160-215, wherein [D] replaces positions 593-595 (e.g., T593, G594, W595), numbered according to the amino acid sequence of SEQ ID NO: 138.
217. The isolated AAV particle of any one of embodiments 160-216, wherein [D] is present immediately subsequent to position 592, and replaces positions 593-595 (e.g., T593, G594, W595), numbered according to the amino acid sequence of SEQ ID NO: 138.
218. The isolated AAV particle of any one of embodiments 160-217, wherein [D] corresponds to positions 600-602 of SEQ ID NO: 5, 8, or 3636.
219. The isolated AAV particle of any one of embodiments 160-218, wherein [C][D] is present immediately subsequent to position 588, numbered according to SEQ ID NO: 138.
220. The isolated AAV particle of any one of embodiments 160-219, wherein [C][D] replaces positions 589-595 (e.g., A589, Q590, A591, Q592, T593, G594, W595), numbered according to the amino acid sequence of SEQ ID NO: 138.
221. The isolated AAV particle of any one of embodiments 160-220, wherein [C][D] is present immediately subsequent to 588, and replaces positions 589-595 (e.g., A589, Q590, A591, Q592, T593, G594, W595), numbered according to the amino acid sequence of SEQ ID NO: 138.
222. The isolated AAV particle of any one of embodiments 160-221, wherein [C][D] corresponds to positions 596-602 of SEQ ID NO: 5, 8, or 3636.
223. The isolated AAV particle of any one of embodiments 160-222, wherein [A][B][C][D] is present immediately subsequent to position 586, numbered according to SEQ ID NO: 5, 8, 138, or 3636.
224. The isolated AAV particle of any one of embodiments 160-223, wherein [A][B][C][D] replaces positions 587-595 (e.g., A587, Q588, A589, Q590, A591, Q592, T593, G594, W595), numbered according to SEQ ID NO: 138.
225. The isolated AAV particle of any one of embodiments 160-224, wherein [A][B][C][D] is present immediately subsequent to position 586 and replaces positions 587-595 (e.g., A587, Q588, A589, Q590, A591, Q592, T593, G594, W595), numbered according to SEQ ID NO: 138.
226. The isolated AAV particle of any one of embodiments 160-225, wherein [A][B][C][D] corresponds to positions 587-602 of SEQ ID NO: 5, 8, or 3636.
227. The isolated AAV particle of any one of embodiments 171-226, wherein [E] is present immediately subsequent to position 595, relative to a reference sequence numbered according to the amino acid sequence of SEQ ID NO: 138.
228. The isolated AAV particle of any one of embodiments 171-227, wherein [E] replaces positions 596-598 (e.g., V596, Q597, N598), numbered according to the amino acid sequence of SEQ ID NO: 138.
229. The isolated AAV particle of any one of embodiments 171-228, wherein [E] is present immediately subsequent to position 595, and replaces positions 596-598 (e.g., V596, Q597, N598), numbered according to the amino acid sequence of SEQ ID NO: 138.
230. The isolated AAV particle of any one of embodiments 171-229, wherein [E] corresponds to positions 603 to 605 of SEQ ID NO: 5, 8, or 3636.
231. The isolated AAV particle of any one of embodiments 171-230, wherein [A][B][C][D][E] is present immediately subsequent to position 586, relative to a reference sequence numbered according to the amino acid sequence of SEQ ID NO: 5, 8, 138, or 3636.
232. The isolated AAV particle of any one of embodiments 171-231, wherein [A][B][C][D][E] replaces positions 587-598 (e.g., A587, Q588, A589, Q590, A591, Q592, T593, G594, W595, V596, Q597, N598), numbered according to SEQ ID NO: 138.
233. The isolated AAV particle of any one of embodiments 171-232, wherein [A][B][C][D][E] is present immediately subsequent to position 586 and replaces positions 587-598 (e.g., A587, Q588, A589, Q590, A591, Q592, T593, G594, W595, V596, Q597, N598), numbered according to SEQ ID NO: 138.
234. The isolated AAV particle of any one of embodiments 171-233, wherein [A][B][C][D][E] corresponds to positions 587-605 of SEQ ID NO: 5, 8, or 3636.
235. The isolated AAV particle of any one of embodiments 132-234, wherein the AAV capsid variant comprises from N-terminus to C-terminus, [A][B].
236. The isolated AAV particle of any one of embodiments 144-235, wherein the AAV capsid variant comprises from N-terminus to C-terminus, [A][B][C].
237. The isolated AAV particle of any one of embodiments 160-236, wherein the AAV capsid variant comprises from N-terminus to C-terminus, [A][B][C][D].
238. The isolated AAV particle of any one of embodiments 171-237, wherein the AAV capsid variant comprises from N-terminus to C-terminus, [A][B][C][D][E].
239. An isolated, e.g., recombinant, AAV particle comprising an AAV capsid variant and a nucleic acid comprising a transgene encoding an antibody molecule which binds to tau (e.g., human tau), wherein the AAV capsid variant comprises PLNGAVHLY (SEQ ID NO: 3648) and optionally wherein the AAV capsid variant further comprises one, two, or all of an amino acid other than T at position 593 (e.g., A, L, R, V, C, I, K, M, N, P, Q, S), an amino acid other than G at position 594 (e.g., M, S, A, Q, V, T, L, P, H, K, N, I, Y, or R), and/or an amino acid other than W at position 595 (e.g., S, P, T, A, G, L, Q, H, N, R, K, V, E, F, M, C, or Y), relative to a reference sequence numbered according to the amino acid sequence of SEQ ID NO: 138.
240. The isolated AAV particle of embodiment 239, wherein:
   (i) the amino acid T, A, L, R, V, C, I, K, M, N, P, Q, or S is present at position 593, numbered according to the amino acid sequence of SEQ ID NO: 138;
   (ii) the amino acid G, M, S, A, Q, V, T, L, P, H, K, N, I, Y, or R is present at position 594, numbered according to the amino acid sequence of SEQ ID NO: 138; and/or
   (iii) the amino acid W, S, P, T, A, G, L, Q, H, N, R, K, V, E, F, M, C, or Y is present at position 595, numbered according to the amino acid sequence of SEQ ID NO: 138.
241. An isolated, e.g., recombinant, AAV particle comprising an AAV capsid variant and a nucleic acid comprising a transgene encoding an antibody molecule which binds to tau (e.g., human tau), wherein the AAV capsid variant comprises an amino sequence comprising the amino acid sequence of PLNGAVHLY (SEQ ID NO: 3648); and which further comprises one, two, three, or all of:
   (i) the amino acid T, A, L, R, V, C, I, K, M, N, P, Q, or S at position 593 numbered according to SEQ ID NO: 138 or at position 600 numbered according to SEQ ID NO: 5, 8, or 3636;
   (ii) the amino acid G, M, S, A, Q, V, T, L, P, H, K, N, I, Y, or R at position 594 numbered according to SEQ ID NO: 138 or at position 601 numbered according to SEQ ID NO: 5, 8, or 3636; and/or
   (iii) the amino acid W, S, P, T, A, G, L, Q, H, N, R, K, V, E, F, M, C, or Y at position 595 numbered according to SEQ ID NO: 138 or at position 602 numbered according to SEQ ID NO: 5, 8, or 3636;
   optionally, provided that the amino acids at positions 593-595, relative to a reference sequence numbered according to the amino acid sequence of SEQ ID NO: 138, does not comprise the amino acid sequence of TGW.
242. The isolated AAV particle of any one of embodiments 239-241, wherein the AAV capsid variant comprises:
   (i) the amino acid L at position 593 numbered according to SEQ ID NO: 138 or at position 600 numbered according to SEQ ID NO: 5, 8, or 3636;
   (ii) the amino acid S at position 594 numbered according to SEQ ID NO: 138 or at position 601 numbered according to SEQ ID NO: 5, 8, or 3636; and/or
   (iii) the amino acid P at position 595 numbered according to SEQ ID NO: 138 or at position 602 numbered according to SEQ ID NO: 5, 8, or 3636.
243. The isolated AAV particle of any one of embodiments 239-242, wherein the to the AAV capsid variant comprises the amino acid sequence of SEQ ID NO: 138, comprise the amino acid sequence of TMS, ASP, LGS, LSS, RST, TAA, TAG, TAL, TAS, TGT, TMA, TQP, TSA, TSP, TST, TVA, TVS, VMS, VSP, VSS, VTP, TGP, VAS, AAA, AAP, AGP, AGW, ALP, AMS, AQP, ATG, ATP, ATQ, ATS, AVG, CSP, CTP, IAA, IAG, IAS, IGG, IGS, IGW, ILG, IPP, IQP, IQS, ISG, ISP, ISS, ITP, KGW, KSS, LAA, LAG, LAH, LAN, LAP, LAQ, LAR, LAS, LAT, LGP, LGQ, LGT, LHP, LKS, LLP, LMA, LMG, LMP, LMQ, LMS, LNP, LPP, LQP, LQQ, LQR, LSA, LSG, LSH, LSK, LSN, LSP, LSQ, LSR, LST, LTA, LTN, LTP, LTS, LVG, MAP, NAL, NAQ, NAS, NLP, NMQ, NQP, NSP, NTP, NTQ, NTT, NVQ, PGW, QAP, QSP, RAA, RAG, RAQ, RAS, RGG, RGS, RGW, RIA, RIG, RIP, RLG, RLP, RLS, RLT, RML, RMS, RNS, RPG, RPP, RQG, RQL, RQP, RQQ, RQS, RSA, RSG, RSP, RSQ, RSS, RSV, RTA, RTG, RTL, RTP, RTQ, RTS, RTT, RVE, RYS, SAA, SAF, SAG, SAK, SAL, SAM, SAN, SAP, SAQ, SAV, SGP, SLP, SMA, SMG, SMQ, SMS, SPP, SQL, SQM, SQP, SQQ, SSL, SSM, SSP, SSR, SSS, STP, STQ, STS, SVA, SVG, SVQ, SVS, TAK, TAM, TAN, TAP, TAQ, TAT, TAV, TAW, TGA, TGC, TGF, TGG, TGL, TGM, TGQ, TGR, TGS, TGV, TGY, TKA, TKP, TKQ, TKS, TLA, TLG, TLP, TLQ, TLR, TLS, TMG, TMK, TMM, TMN, TMP, TMQ, TMT, TNA, TNG, TNQ, TNS, TPA, TPG, TPP, TPQ, TPR, TPS, TPT, TPW, TQA, TQH, TQK, TQL, TQM, TQN, TQQ, TQR, TQT, TQW, TRW, TSG, TSH, TSK, TSL, TSM, TSN, TSQ, TSS, TSV, TSW, TTA, TTG, TTK, TTP, TTQ, TTR, TTS, TTT, TVG, TVQ, TVW, TYA, TYS, VAA, VAG, VAK, VAN, VAQ, VAT, VGG, VGK, VGN, VGS, VHP, VKA, VKP, VKQ, VLG, VLP, VLS, VMA, VMP, VMQ, VNA, VNG, VNS, VPA, VPP, VPQ, VQA, VQG, VQN, VQP, VQQ, VQS, VQT, VRP, VSA, VSG, VSN, VSQ, VSR, VST, VTA, VTG, VTK, VTS, or VAP at positions 593-595 numbered according to SEQ ID NO: 138 or at positions 600-602 numbered according to SEQ ID NO: 5, 8, or 3636.
244. The isolated AAV particle of any one of embodiments 239-243, wherein the AAV capsid variant comprises the amino acid sequence LSP at positions 593-595 numbered according to SEQ ID NO: 138 or at positions 600-602 numbered according to SEQ ID NO: 5, 8, or 3636.
245. The isolated AAV particle of any one of embodiments 239-244, wherein the AAV capsid variant further comprises one, two, three, or all of an amino acid other than A at position 589 (e.g., D, S, or T), an amino acid other than Q at position 590 (e.g., K, H, L, P, or R), an amino acid other than A at position 591 (e.g., P or E), and/or an amino acid other than Q at position 592 (e.g., H, K, or P).
246. The isolated AAV particle of embodiment 245, wherein:
   (i) the amino acid A, D, S, or T at position 589, numbered according to SEQ ID NO: 138;
   (ii) the amino acid Q, K, H, L, P, or R at position 590, numbered according to SEQ ID NO: 138;
   (iii) the amino acid A, E, or P at position 591, numbered according to SEQ ID NO: 138; and/or
   (iv) the amino acid Q, H, K, or P at position 592, numbered according to SEQ ID NO: 138.
247. The isolated AAV particle of embodiment 245 or 246, wherein the AAV capsid variant comprises the amino acid sequence of:
   (i) AHAQ (SEQ ID NO: 4742), AKAQ (SEQ ID NO: 4741), ALAQ (SEQ ID NO: 4749), APAQ (SEQ ID NO: 4745), AQAH (SEQ ID NO: 4747), AQAK (SEQ ID NO: 4746), AQAP (SEQ ID NO: 4743), AQAQ (SEQ ID NO: 4737), AQEQ (SEQ ID NO: 4748), AQPQ (SEQ ID NO: 4739), ARAQ (SEQ ID NO: 4750), DQAQ (SEQ ID NO: 4744), SQAQ (SEQ ID NO: 4738), or TQAQ (SEQ ID NO: 4751) at positions 589-592 numbered according to SEQ ID NO: 138 or at positions 596-599 numbered according to SEQ ID NO: 5, 8, or 3636; or
   (ii) AKAQ (SEQ ID NO: 4741), AQAQ (SEQ ID NO: 4737), AQPQ (SEQ ID NO: 4739), DQAQ (SEQ ID NO: 4744), or SQAQ (SEQ ID NO: 4738) at positions 589-592 numbered according to SEQ ID NO: 138 or at positions 596-599 numbered according to SEQ ID NO: 5, 8, or 3636.
248. The isolated AAV particle of any one of embodiments 239-247, wherein the AAV capsid variant further comprises one, two, or all of an amino acid other than V at position 596 (e.g., G, F, D, L, A, I, or E), an amino acid other than Q at position 597 (e.g., K, R, H, E, L, or P), and/or an amino acid other than N at position 598 (e.g., H, K, T, I, S, D, P, or Y), relative to a reference sequence numbered according to the amino acid sequence of SEQ ID NO: 138.
249. The isolated AAV particle of any one of embodiments 248, wherein:
   (i) the amino acid V, G, F, D, L, A, I, or E at position 596 numbered according to SEQ ID NO: 138 or at position 603 numbered according to SEQ ID NO: 5, 8, or 3636;
   (ii) the amino acid Q, K, R, H, E, L, or P at position 597, numbered according to SEQ ID NO: 138 or at position 604 numbered according to SEQ ID NO: 5, 8, or 3636; and/or
   (iii) the amino acid N, H, K, T, I, S, D, P, or Y at position 598 numbered according to SEQ ID NO: 138 or at position 605 numbered according to SEQ ID NO: 5, 8, or 3636.
250. An isolated, e.g., recombinant, AAV particle comprising an AAV capsid variant and a nucleic acid comprising a transgene encoding a transgene encoding an antibody molecule which binds to tau, wherein the AAV capsid variant comprises PLNGAVHLY (SEQ ID NO: 3648), and optionally wherein the AAV capsid variant further comprises one, two, or all of an amino acid other than V at position 596 (e.g., G, F, D, L, A, I, or E), an amino acid other than Q at position 597 (e.g., K, R, H, E, L, or P), and/or an amino acid other than N at position 598 (e.g., H, K, T, I, S, D, P, or Y), numbered according to SEQ ID NO: 138.
251. An isolated, e.g., recombinant, AAV particle comprising an AAV capsid variant and a nucleic acid comprising a transgene encoding a transgene encoding an antibody molecule which binds to tau, wherein the AAV capsid variant comprises PLNGAVHLY (SEQ ID NO: 3648), and further comprising one, two, or all of:
   (i) V, G, F, D, L, A, I, or E at position 596 numbered according to SEQ ID NO: 138 or at position 603 numbered according to SEQ ID NO: 5, 8, or 3636;
   (ii) Q, K, R, H, E, L, or P at position 597 numbered according to SEQ ID NO: 138 or at position 604 numbered according to SEQ ID NO: 5, 8, or 3636; and/or
   (iii) N, H, K, T, I, S, D, P, or Y at position 598 numbered according to SEQ ID NO: 138 or at position 605 numbered according to SEQ ID NO: 5, 8, or 3636;
   optionally, provided that the amino acids at positions 596-598 numbered according to the amino acid sequence of SEQ ID NO: 138 or positions 603-605 of SEQ ID NO: 5, 8, or 3636, does not comprise the amino acid sequence of VQN.
252. The isolated AAV particle of any one of embodiments 239-251, which comprises the amino acid P at position 597 numbered according to SEQ ID NO: 138 or at position 604 numbered according to SEQ ID NO: 5, 8, or 3636.
253. The isolated AAV particle of any one of embodiments 239-252, which comprises the amino acid K at position 597 numbered according to SEQ ID NO: 138 or at position 604 numbered according to SEQ ID NO: 5, 8, or 3636.
254. The isolated AAV particle of any one of embodiments 248-253, wherein the AAV capsid variant comprises the amino acid sequence of:
   (i) GQN, VQH, VQK, VQT, VQN, FQN, VKN, VQI, DQN, LQN, VQS, VRN, AQN, IQN, VHN, VLN, VEN, VQD, DKN, EQN, FQK, GHN, GQT, VKK, VKT, VPH, VPN, VQP, or VQY at positions 596-598 numbered according to SEQ ID NO: 138 or positions 603-605 numbered according to SEQ ID NO: 5, 8, or 3636; or
   (ii) VQN, VQT, VQK, DQN, VQH, FQN, AQN, VLN, LQN, VQI, VQS, EQN, GQT, VPN, VQD, VQP, VRN, or VKN at positions 596-598 numbered according to SEQ ID NO: 138 or positions 603-605 numbered according to SEQ ID NO: 5, 8, or 3636.
255. The isolated AAV particle of any one of embodiments 248-254, which comprises the amino acid sequence of VKN, VPN, or VQN at positions 596-598 numbered according to SEQ ID NO: 138 or positions 603-605 numbered according to SEQ ID NO: 5, 8, or 3636.
256. The isolated AAV particle of any one of embodiments 248-255, which comprises the amino acid sequence of VEN or VHN at positions 596-598 numbered according to SEQ ID NO: 138 or positions 603-605 numbered according to SEQ ID NO: 5, 8, or 363.
257. The isolated AAV particle of any one of embodiments 239-256, wherein the AAV capsid variant comprises:
   (i) the amino acid sequence of any of SEQ ID NOs: 143, 148, 149, 151, 153-158, 160-163, 166, 168, 170, 171, 173-175, 177-179, 181, 182, 184-188,191-197, 199-210,212-215, 217-225, 227-231, 233, 234, 236-240, 243-262, 265, 267, 268, 270-277, 279, 282, 284, 285, 286, 288-293, 295, 296, 298, 300-314, 316-318, 320-327, 329, 331, 332, 334, 336-344, 346-350, 352-354, 356-367, 369, 371-380, 382-385, 387, 392-394, 396, 397, 399-401, 404-411, 413-415, 417, 419-429, 432, 433, 435-437, 438, 440-442, 444-447, 450-453, 456, 458-461, 464, 465, 467-469, 471-478, 480-483, 487-495, 497, 498, 500-503, 505, 507-512, 515-517, 522-525, 528-532, 534-539, 542-545, 547, 551-554, 558-561, 563-568, 570, 573, 574, 576, 579, 581, 582, 584, 586, 587, 592-596, 598, 601, 604-607, 610, 612, 614-619, 624-629, 631, 633-636, 640, 641, 645, 646, 649, 650, 658, 663, 664, 666, 668, 669, 672, 673, 675, 679, 683, 684, 686, 688, 689, 691, 693, 695, 697, 699, 700, 704, 705, 709-712, 720, 722, 726-731, 733, 736, 740, 745, 749, 750-752, 754, 755, 757, 758, 760-765, 767, 768, 771, 778, 780, 783-787, 792, 794, 797, 799-802, 804, 817, 818, 821, 824, 828, 831, 832, 834-837, 840-845, 847, 848, 851-853, 855, 858, 861, 862, 865, 869, 870-872, 874, 876, 882, 883, 887, 889, 890, 892-895, 897, 901, 903, 904, 905, 907, 910, 911, 913, 915, 919, 920, 923, 924, 926, 927, 929, 931-933, 935, 937, 940, 941, 943, 945-949, 953, 955, 957, 958, 960, 962, 964, 965, 971, 973, 974, 977, 986, 988, 989, 992, 994, 997, 998, 1000, 1004, 1007, 1013, 1015, 1017, 1018, 1020, 1025, 1027, 1029, 1030, 1031, 1033-1035, 1037-1039, 1043, 1046, 1049, 1052, 1056, 1057, 1059, 1062, 1065, 1067, 1068, 1070, 1073, 1075, 1077-1079, 1083-1087, 1089, 1090, 1094, 1100, 1101, 1103, 1106, 1107, 1110, 1111, 1112, 1114, 1115, 1117, 1119, 1125, 1126, 1129, 1132, or 1133;
   (ii) an amino acid sequence comprising any portion of an amino acid sequence in (i), e.g., any 2, 3, 4, 5, 6, 7, 8, 9, 10, 11, 12, 13, 14, 15, 16, 17, or 18 amino acids, e.g., consecutive amino acids, thereof;
   (iii) an amino acid sequence comprising at least one, two, or three but no more than four modifications, e.g., substitutions (e.g., conservative substitutions), insertions, or deletions, relative to any of the amino acid sequences in (i); or
   (iv) an amino acid sequence comprising at least one, two, or three but no more than four different amino acids, relative to any one of the amino acid sequences in (i).
258. The isolated AAV particle of any one of embodiments 239-257, wherein the AAV capsid variant comprises:
   (i) the amino acid sequence of any of SEQ ID NOs: 143, 148-151, 153-158, 160-163, 166, 168, 170, 171, 173-175, 177-179, 181, 182, 184-188, 191-197, 199-210, 212-215, 217-225, 227-231, 233, 234, 236-240, 243-262, 265, 267, 268, 270-277, 279, 282, 284-286, 288-293, 295, 296, 298, 300-314, 316-318, 320-327, 329, 331, 332, 334, 336, 337-339, 340-344, 346-350, 352-354, 356-365, 367, 369, 371-380, 382-385, 387, 392-394, 396, 397, 399-401, 404-411, 413-415, 417, 419, 420-429, 432, 433, 435-438, 440-442, 444-447, 450-453, 456, 458, 459, 460, 461, 464, 465, 467-469, or 471-476;
   (ii) an amino acid sequence comprising any portion of an amino acid sequence in (i), e.g., any 2, 3, 4, 5, 6, 7, 8, 9, 10, 11, 12, 13, 14, 15, 16, 17, or 18 amino acids, e.g., consecutive amino acids, thereof;
   (iii) an amino acid sequence comprising at least one, two, or three but no more than four modifications, e.g., substitutions (e.g., conservative substitutions), insertions, or deletions, relative to any of the amino acid sequences in (i); or
   (iv) an amino acid sequence comprising at least one, two, or three but no more than four different amino acids, relative to any one of the amino acid sequences in (i).
259. The isolated AAV particle of any one of embodiments 239-258, wherein the amino acid sequence of PLNGAVHLY (SEQ ID NO: 3648) is present in loop VIII.
260. The isolated AAV particle of any one of embodiments 239-259, wherein the amino acid sequence of PLNGAVHLY (SEQ ID NO: 3648) is present immediately subsequent to position 586, relative to a reference sequence numbered according to the amino acid sequence of SEQ ID NO: 138.
261. The isolated AAV particle of any one of embodiments 239-260, wherein the amino acid sequence of PLNGAVHLY (SEQ ID NO: 3648) replaces positions 587 and 588 (e.g., A587 and Q588), numbered according to SEQ ID NO: 138.
262. The isolated AAV particle of any one of embodiments 239-261, wherein the amino acid sequence of PLNGAVHLY (SEQ ID NO: 3648) is present immediately subsequent to position 586 and replaces positions 587 and 588 (e.g., A587 and Q588), numbered according to SEQ ID NO: 138.
263. The isolated AAV particle of any one of embodiments 239-262, wherein the amino acid sequence of PLNGAVHLY (SEQ ID NO: 3648) corresponds to positions 587-595 of SEQ ID NO: 5, 8, or 3636.
264. An isolated, e.g., recombinant, AAV particle comprising an AAV capsid variant and a nucleic acid comprising a transgene encoding an antibody molecule which binds to tau (e.g., human tau), wherein the AAV capsid variant comprises X1-X2-X3-X4-X5-X6-X7-X8-X9-X10-X11-X12-X13-X14-X15-X16-X17-X18-X19, wherein:
   (i) X1 is: P, A, D, E, F, G, H, K, L, N, Q, R, S, T, or V;
   (ii) X2 is: L, D, E, F, H, I, M, N, P, Q, R, S, or V;
   (iii) X3 is: N, A, D, E, G, H, I, K, Q, S, T, V, or Y;
   (iv) X4 is: G, A, C, D, E, P, Q, R, S, T, V, or W;
   (v) X5 is: A, C, D, E, F, G, H, I, K, N, P, Q, R, S, T, V, W, or Y;
   (vi) X6 is: V, A, C, D, E, F, G, H, I, K, L, M, N, Q, R, S, T, or Y;
   (vii) X7 is: H, A, D, E, G, I, K, L, M, N, P, Q, R, S, T, V, or Y;
   (viii) X8 is: L, A, D, E, F, G, H, I, K, M, N, P, Q, R, S, T, V, or Y;
   (ix) X9 is: Y, A, C, D, E, F, G, H, I, K, L, M, N, Q, R, S, T, V, or W;
   (x) X10 is: A, C, D, E, F, G, H, I, K, L, N, P, Q, R, S, T, V, or; Y;
   (xi) X11 is: Q, A, D, E, H, K, L, P, R, or T;
   (xii) X12 is: A, D, E, G, H, L, N, P, Q, R, S, T, or V;
   (xiii) X13 is: Q, E, H, K, L, P, R, or T;
   (xiv) X14 is: T, A, C, D, E, F, G, H, I, K, L, M, N, P, Q, R, S, V, W, or Y;
   (xv) X15 is: G, A, C, D, E, F, H, I, K, L, M, N, P, Q, R, S, T, V, W, or Y;
   (xvi) X16 is: W, A, C, D, E, F, G, H, I, K, L, M, N, P, Q, R, S, T, V, or Y;
   (xvii) X17 is: V, A, D, E, F, G, H, I, or L;
   (xviii) X18 is: Q, E, H, K, L, P, or R; and/or
   (xix) X19 is: N, D, H, I, K, P, S, T, or Y.
265. The isolated AAV particle of embodiment 264, wherein:
   (i) X1 is: P, Q, A, S, T, R, H, L, or K;
   (ii) X2 is: L, I, V, H, or R;
   (iii) X3 is: N, D, K, Y, or I;
   (iv) X4 is: G, S, R, C, or A;
   (v) X5 is: A, S, G, N, T, D, Y, Q, V, or C;
   (vi) X6 is: V, I, L, A, F, D, or G;
   (vii) X7 is: H, N, Q, P, D, L, R, or Y;
   (viii) X8 is: L, H, V, I, or R;
   (ix) X9 is Y;
   (x) X10 is: A, D, S, or T;
   (xi) X11 is: Q, K, H, L, P, or R;
   (xii) X12 is: A, P, E, or S;
   (xiii) X13 is: Q, K, H, or P;
   (xiv) X14 is: L, T, V, S, R, I, A, N, C, P, Q, M, or K;
   (xv) X15 is: S, G, M, T, A, K, Q, V, I, R, N, P, L, H, Y;
   (xvi) X16 is: P, W, S, K, Q, G, C, R, A, N, T, V, M, H, L, E, F, or Y;
   (xvii) X17 is: V, D, F, A, E, L, G, or I;
   (xviii) X18 is: Q, R, P, K, L, H, or E; and/or
   (xix) X19 is: N, H, D, S, T, P, K, I, or Y.
266. The isolated AAV particle of embodiment 264 or 265, wherein:
   (i) X1 is: P, A, S, Q, or T;
   (ii) X2 is L or I;
   (iii) X3 is N or D;
   (iv) X4 is G or S;
   (v) X5 is: A, S, G, N, or T;
   (vi) X6 is V;
   (vii) X7 is H;
   (viii) X8 is: L, H, V, or I
   (ix) X9 is Y;
   (x) X10 is: A, D, or S;
   (xi) X11 is Q or K;
   (xii) X12 is A or P;
   (xiii) X13 is Q;
   (xiv) X14 is: L, T, V, S, R, I, A, N, C, P, Q, or M;
   (xv) X15 is: S, G, M, T, A, K, Q, V, I, R, N, P, L, or H;
   (xvi) X16 is: P, W, S, K, Q, G, C, R, A, N, T, V, M, H, L, or E;
   (xvii) X17 is: V, D, F, A, E, L, or G;
   (xviii) X18 is: Q, R, P, K, or L; and/or
   (xix) X19 is: N, H, D, S, T, P, K, or I.
267. The isolated AAV particle of any one of embodiments 264-266, wherein X1-X2-X3-X4-X5-X6-X7-X8-X9-X10-X11-X12-X13-X14-X15-X16-X17-X18-X19 is present immediately subsequent to position 586, numbered according to SEQ ID NO: 5, 8, 138, or 3636.
268. The isolated AAV particle of any one of embodiments 264-267, wherein X1-X2-X3-X4-X5-X6-X7-X8-X9-X10-X11-X12-X13-X14-X15-X16-X17-X18-X19 replaces positions 587-598 (e.g., A587, Q588, A589, Q590, A591, Q592, T593, G594, W595, V596, Q597, N598), numbered according to SEQ ID NO: 138.
269. The isolated AAV particle of any one of embodiments 264-268, wherein X1-X2-X3-X4-X5-X6-X7-X8-X9-X10-X11-X12-X13-X14-X15-X16-X17-X18-X19 is present immediately subsequent to position 586 and replaces positions 587-598 (e.g., A587, Q588, A589, Q590, A591, Q592, T593, G594, W595, V596, Q597, N598), numbered according to SEQ ID NO: 138.
270. The isolated AAV particle of any one of embodiments 264-269, wherein X1-X2-X3-X4-X5-X6-X7-X8-X9-X10-X11-X12-X13-X14-X15-X16-X17-X18-X19 corresponds to positions 587-605 of SEQ ID NO: 5, 8, or 3636.
271. An isolated, e.g., recombinant, AAV particle comprising an AAV capsid variant and a nucleic acid comprising a transgene encoding an antibody molecule which binds to tau (e.g., human tau), wherein the AAV capsid variant comprises:
   (a) the amino acid sequence of any one of SEQ ID NOs: 139-1138;
   (b) an amino acid sequence comprising at least 3, 4, 5, 6, 7, 8, 9, 10, 11, 12, 13, 14, 15, 16, 17, or 18 consecutive amino acids from any one of SEQ ID NOs: 139-1138; or
   (c) an amino acid sequence comprising at least one, two, or three but no more than four different amino acids, relative to the amino acid sequence of any one of SEQ ID NOs: 139-1138;
   (d) an amino sequence comprising at least one, two, or three but no more than four modifications, e.g., substitutions (e.g., conservative substitutions), insertions, or deletions, relative to the amino acid sequence of any one of SEQ ID NOs: 139-1138;
   optionally wherein the AAV capsid variant does not comprise the amino acid sequence of TGW at positions 593-595, relative to a reference sequence numbered according to the amino acid sequence of SEQ ID NO: 138.
272. An isolated, e.g., recombinant, AAV particle comprising an AAV capsid variant and a nucleic acid comprising a transgene encoding an antibody molecule which binds to tau (e.g., human tau), wherein the AAV capsid variant comprises:
   (a) the amino acid sequence of any one of SEQ ID NOs: 139-476;
   (b) an amino acid sequence comprising at least 3, 4, 5, 6, 7, 8, 9, 10, 11, 12, 13, 14, 15, 16, 17, or 18 consecutive amino acids from any one of SEQ ID NOs: 139-476; or
   (c) an amino acid sequence comprising at least one, two, or three but no more than four different amino acids, relative to the amino acid sequence of any one of SEQ ID NOs: 139-476;
   (d) an amino sequence comprising at least one, two, or three but no more than four modifications, e.g., substitutions (e.g., conservative substitutions), insertions, or deletions, relative to the amino acid sequence of any one of SEQ ID NOs: 139-476;
   optionally wherein the AAV capsid variant does not comprise the amino acid sequence of TGW at positions 593-595, relative to a reference sequence numbered according to the amino acid sequence of SEQ ID NO: 138.
273. An isolated, e.g., recombinant, AAV particle comprising an AAV capsid variant and a nucleic acid comprising a transgene encoding an antibody molecule which binds to tau (e.g., human tau), wherein the AAV capsid variant comprises:
   (a) the amino acid sequence of any one of SEQ ID NOs: 140, 142-144, 148-150, 154-158, 160, 161, 163, 165, 166, 168, 170, 171, 173-175, 177-179, 181, 182, 184-197, 199-214, 218-222, 224, 225, 227-241, 243-253, 255-262, 265, 267, 268, 270, 271, 273, 274, 276, 277, 279, 282, 284-286, 288-296, 300-310, 312, 315, 317, 318, 320-323, 326, 327, 331, 332, 334, 336, 337, 339, 340, 341, 343, 344, 346, 349, 351, 352, 356-363, 365-367, 369, 370, 372-376, 378-381, 383-389, 392, 393, 395, 397-400, 404, 407, 408, 411, 412, 415, 417, 420-430, 432, 433, 435-438, 441, 442, 446-448, 451-453, 456, 458, 460, 461, 465, 467-469, 471-473, 475, 476, 478, 480, 482, 485, 488, 490, 492, 493, 495, 498, 500-503, 505, 507, 509, 510, 517, 522-526, 528, 535-538, 540, 543-545, 547, 551, 552, 557, 559, 561, 564, 568, 570, 572-574, 577, 585-588, 592-594, 596, 601, 602, 605, 612, 616, 619, 622, 624, 627, 628, 635, 640, 641, 646, 658, 660, 665, 666, 675, 678, 680, 683, 684, 689, 693, 695, 707, 711, 718, 719, 724, 727, 735, 740, 748, 751, 755, 758, 759, 765, 766, 768, 778, 783, 787, 791, 797, 801, 804, 817, 821, 832, 841, 852, 856, 861, 862, 864, 894, 906, 911, 913, 924, 929, 945, 959, 961, 970, 975, 980, 983, 988, 992, 1009, 1015, 1019, 1027, 1032, 1036, 1038, 1047, 1051, 1061, 1077, 1081, 1095, or 1113;
   (b) an amino acid sequence comprising at least 3, 4, 5, 6, 7, 8, 9, 10, 11, 12, 13, 14, 15, 16, 17, or 18 consecutive amino acids from any one of the amino acid sequences in (i); or
   (c) an amino acid sequence comprising at least one, two, or three but no more than four different amino acids, relative to the amino acid sequence of any one of the amino acid sequences in (i);
   (d) an amino sequence comprising at least one, two, or three but no more than four modifications, e.g., substitutions (e.g., conservative substitutions), insertions, or deletions, relative to the amino acid sequence of any one of the amino acid sequences in (i);
   optionally wherein the AAV capsid variant does not comprise the amino acid sequence of TGW at positions 593-595, relative to a reference sequence numbered according to the amino acid sequence of SEQ ID NO: 138.
274. The isolated AAV particle of embodiment 271, wherein the AAV capsid variant comprises an amino acid sequence comprising at least 4, 5, 6, 7, 8, 9, 10, 11, 12, 13, 14, 15, 16, 17, or 18 consecutive amino acids from any one of SEQ ID NOs: 139-1138.
275. The AAV capsid variant of any one of embodiments 271, 272, or 274, wherein the AAV capsid variant comprises an amino acid sequence comprising at least 4, 5, 6, 7, 8, 9, 10, 11, 12, 13, 14, 15, 16, 17, or 18 consecutive amino acids from SEQ ID NO: 314.
276. The AAV capsid variant of any one of embodiments 271 or 274, wherein the AAV capsid variant comprises an amino acid sequence comprising at least 4, 5, 6, 7, 8, 9, 10, 11, 12, 13, 14, 15, 16, 17, or 18 consecutive amino acids from SEQ ID NO: 566.
277. The isolated AAV particle of embodiment 271 or 272, comprising an amino acid sequence comprising at least 4, 5, 6, 7, 8, 9, 10, 11, 12, 13, 14, 15, 16, 17, or 18 consecutive amino acids from any one of SEQ ID NOs: 139-476.
278. The isolated AAV particle of embodiment 271 or 273, wherein the AAV capsid variant comprises an amino acid sequence comprising at least 4, 5, 6, 7, 8, 9, 10, 11, 12, 13, 14, 15, 16, 17, or 18 consecutive amino acids from any one of SEQ ID NOs: 140, 142-144, 148-150, 154-158, 160, 161, 163, 165, 166, 168, 170, 171, 173-175, 177-179, 181, 182, 184-197, 199-214, 218-222, 224, 225, 227-241, 243-253, 255-262, 265, 267, 268, 270, 271, 273, 274, 276, 277, 279, 282, 284-286, 288-296, 300-310, 312, 315, 317, 318, 320-323, 326, 327, 331, 332, 334, 336, 337, 339, 340, 341, 343, 344, 346, 349, 351, 352, 356-363, 365-367, 369, 370, 372-376, 378-381, 383-389, 392, 393, 395, 397-400, 404, 407, 408, 411, 412, 415, 417, 420-430, 432, 433, 435-438, 441, 442, 446-448, 451-453, 456, 458, 460, 461, 465, 467-469, 471-473, 475, 476, 478, 480, 482, 485, 488, 490, 492, 493, 495, 498, 500-503, 505, 507, 509, 510, 517, 522-526, 528, 535-538, 540, 543-545, 547, 551, 552, 557, 559, 561, 564, 568, 570, 572-574, 577, 585-588, 592-594, 596, 601, 602, 605, 612, 616, 619, 622, 624, 627, 628, 635, 640, 641, 646, 658, 660, 665, 666, 675, 678, 680, 683, 684, 689, 693, 695, 707, 711, 718, 719, 724, 727, 735, 740, 748, 751, 755, 758, 759, 765, 766, 768, 778, 783, 787, 791, 797, 801, 804, 817, 821, 832, 841, 852, 856, 861, 862, 864, 894, 906, 911, 913, 924, 929, 945, 959, 961, 970, 975, 980, 983, 988, 992, 1009, 1015, 1019, 1027, 1032, 1036, 1038, 1047, 1051, 1061, 1077, 1081, 1095, 1113.
279. The isolated AAV particle of embodiment 271, wherein the AAV capsid variant comprises an amino acid sequence comprising at least one, two, or three but no more than four different amino acids relative to the amino acid sequence of any one of SEQ ID NOs: 139-1138.
280. The isolated AAV particle of embodiment 271 or 272, wherein the AAV capsid variant comprises an amino acid sequence comprising at least one, two, or three but no more than four different amino acids relative to the amino acid sequence of any one of SEQ ID NOs: 139-476.
281. The isolated AAV particle of embodiment 271 or 273, wherein the AAV capsid variant comprises an amino acid sequence comprising at least one, two, or three but no more than four different amino acids relative to the amino acid sequence of any one of SEQ ID NOs: 140, 142-144, 148-150, 154-158, 160, 161, 163, 165, 166, 168, 170, 171, 173-175, 177-179, 181, 182, 184-197, 199-214, 218-222, 224, 225, 227-241, 243-253, 255-262, 265, 267, 268, 270, 271, 273, 274, 276, 277, 279, 282, 284-286, 288-296, 300-310, 312, 315, 317, 318, 320-323, 326, 327, 331, 332, 334, 336, 337, 339, 340, 341, 343, 344, 346, 349, 351, 352, 356-363, 365-367, 369, 370, 372-376, 378-381, 383-389, 392, 393, 395, 397-400, 404, 407, 408, 411, 412, 415, 417, 420-430, 432, 433, 435-438, 441, 442, 446-448, 451-453, 456, 458, 460, 461, 465, 467-469, 471-473, 475, 476, 478, 480, 482, 485, 488, 490, 492, 493, 495, 498, 500-503, 505, 507, 509, 510, 517, 522-526, 528, 535-538, 540, 543-545, 547, 551, 552, 557, 559, 561, 564, 568, 570, 572-574, 577, 585-588, 592-594, 596, 601, 602, 605, 612, 616, 619, 622, 624, 627, 628, 635, 640, 641, 646, 658, 660, 665, 666, 675, 678, 680, 683, 684, 689, 693, 695, 707, 711, 718, 719, 724, 727, 735, 740, 748, 751, 755, 758, 759, 765, 766, 768, 778, 783, 787, 791, 797, 801, 804, 817, 821, 832, 841, 852, 856, 861, 862, 864, 894, 906, 911, 913, 924, 929, 945, 959, 961, 970, 975, 980, 983, 988, 992, 1009, 1015, 1019, 1027, 1032, 1036, 1038, 1047, 1051, 1061, 1077, 1081, 1095, or 1113.
282. The isolated AAV particle of embodiment 271, wherein the AAV capsid variant comprises an amino acid sequence comprising at least one, two, or three but no more than four modifications, e.g., substitutions (e.g., conservative substitutions), insertions, or deletions, relative to the amino acid sequence of SEQ ID NOs: 139-1138.
283. The isolated AAV particle of embodiment 271 or 272, wherein the AAV capsid variant comprises an amino acid sequence comprising at least one, two, or three but no more than four modifications, e.g., substitutions (e.g., conservative substitutions), insertions, or deletions, relative to the amino acid sequence of SEQ ID NOs: 139-476.
284. The AAV capsid variant of any one of embodiments 271, 272, 274, 275, or 283, wherein the AAV capsid variant comprises:
   (i) an amino acid sequence comprising at least one, two, or three but no more than four different amino acids relative to the amino acid sequence of SEQ ID NO: 314; or
   (ii) an amino acid sequence comprising at least one, two, or three but no more than four modifications, e.g., substitutions (e.g., conservative substitutions), insertions, or deletions, relative to SEQ ID NO: 314.
285. The AAV capsid variant of any one of embodiments 271, 274, 276, or 283, wherein the AAV capsid variant comprises:
   (i) an amino acid sequence comprising at least one, two, or three but no more than four different amino acids relative to the amino acid sequence of SEQ ID NO: 566; or
   (ii) an amino acid sequence comprising at least one, two, or three but no more than four modifications, e.g., substitutions (e.g., conservative substitutions), insertions, or deletions, relative to SEQ ID NO: 566.
286. The isolated AAV particle of embodiment 271 or 273, wherein the AAV capsid variant comprises an amino acid sequence comprising at least one, two, or three but no more than four modifications, e.g., substitutions (e.g., conservative substitutions), insertions, or deletions, relative to any of SEQ ID NOs: 140, 142-144, 148-150, 154-158, 160, 161, 163, 165, 166, 168, 170, 171, 173-175, 177-179, 181, 182, 184-197, 199-214, 218-222, 224, 225, 227-241, 243-253, 255-262, 265, 267, 268, 270, 271, 273, 274, 276, 277, 279, 282, 284-286, 288-296, 300-310, 312, 315, 317, 318, 320-323, 326, 327, 331, 332, 334, 336, 337, 339, 340, 341, 343, 344, 346, 349, 351, 352, 356-363, 365-367, 369, 370, 372-376, 378-381, 383-389, 392, 393, 395, 397-400, 404, 407, 408, 411, 412, 415, 417, 420-430, 432, 433, 435-438, 441, 442, 446-448, 451-453, 456, 458, 460, 461, 465, 467-469, 471-473, 475, 476, 478, 480, 482, 485, 488, 490, 492, 493, 495, 498, 500-503, 505, 507, 509, 510, 517, 522-526, 528, 535-538, 540, 543-545, 547, 551, 552, 557, 559, 561, 564, 568, 570, 572-574, 577, 585-588, 592-594, 596, 601, 602, 605, 612, 616, 619, 622, 624, 627, 628, 635, 640, 641, 646, 658, 660, 665, 666, 675, 678, 680, 683, 684, 689, 693, 695, 707, 711, 718, 719, 724, 727, 735, 740, 748, 751, 755, 758, 759, 765, 766, 768, 778, 783, 787, 791, 797, 801, 804, 817, 821, 832, 841, 852, 856, 861, 862, 864, 894, 906, 911, 913, 924, 929, 945, 959, 961, 970, 975, 980, 983, 988, 992, 1009, 1015, 1019, 1027, 1032, 1036, 1038, 1047, 1051, 1061, 1077, 1081, 1095, or 1113.
287. The isolated AAV particle of any one of embodiments 271-286, wherein 4, 5, 6, 7, 8, or 9 consecutive amino acids is not PLNG (SEQ ID NO: 3678), PLNGA (SEQ ID NO: 3679), PLNGAV (SEQ ID NO: 3680), PLNGAVHL (SEQ ID NO: 3682), and/or PLNGAVHLY (SEQ ID NO: 3648).
288. The isolated AAV particle of any one of embodiments 271, 274, 279, or 282, wherein the AAV capsid variant comprises the amino acid sequence of any one of SEQ ID NOs: 139-1138.
289. The isolated AAV particle of any one of embodiments 271, 274, 275, 279, or 282, wherein the AAV capsid variant comprises the amino acid sequence of SEQ ID NO: 314.
290. The isolated AAV particle of any one of embodiments 271, 274, 276, 279, or 282, wherein the AAV capsid variant comprises the amino acid sequence of SEQ ID NO: 566.
291. The isolated AAV particle of any one of embodiments 271, 272, 275, 277, 279, 280, 282, or 283, wherein the AAV capsid variant comprises the amino acid sequence of any one of SEQ ID NOs: 139-476.
292. The isolated AAV particle of any one of embodiments 271, 272, 275, 278, 279, 281, 282, or 284, wherein the AAV capsid variant comprises the amino acid sequence of any one of SEQ ID NOs: 140, 142-144, 148-150, 154-158, 160, 161, 163, 165, 166, 168, 170, 171, 173-175, 177-179, 181, 182, 184-197, 199-214, 218-222, 224, 225, 227-241, 243-253, 255-262, 265, 267, 268, 270, 271, 273, 274, 276, 277, 279, 282, 284-286, 288-296, 300-310, 312, 315, 317, 318, 320-323, 326, 327, 331, 332, 334, 336, 337, 339, 340, 341, 343, 344, 346, 349, 351, 352, 356-363, 365-367, 369, 370, 372-376, 378-381, 383-389, 392, 393, 395, 397-400, 404, 407, 408, 411, 412, 415, 417, 420-430, 432, 433, 435-438, 441, 442, 446-448, 451-453, 456, 458, 460, 461, 465, 467-469, 471-473, 475, 476, 478, 480, 482, 485, 488, 490, 492, 493, 495, 498, 500-503, 505, 507, 509, 510, 517, 522-526, 528, 535-538, 540, 543-545, 547, 551, 552, 557, 559, 561, 564, 568, 570, 572-574, 577, 585-588, 592-594, 596, 601, 602, 605, 612, 616, 619, 622, 624, 627, 628, 635, 640, 641, 646, 658, 660, 665, 666, 675, 678, 680, 683, 684, 689, 693, 695, 707, 711, 718, 719, 724, 727, 735, 740, 748, 751, 755, 758, 759, 765, 766, 768, 778, 783, 787, 791, 797, 801, 804, 817, 821, 832, 841, 852, 856, 861, 862, 864, 894, 906, 911, 913, 924, 929, 945, 959, 961, 970, 975, 980, 983, 988, 992, 1009, 1015, 1019, 1027, 1032, 1036, 1038, 1047, 1051, 1061, 1077, 1081, 1095, or 1113.
293. The isolated AAV particle of any one of embodiments 1-292, wherein the AAV capsid variant comprises an amino acid sequence comprising at least 5, 6, 7, 8, or 9 consecutive amino acids from the amino acid sequence of PLNGAVHLY (SEQ ID NO: 3648), wherein:
   (i) the 5 consecutive amino acids comprise PLNGA (SEQ ID NO: 3679);
   (ii) the 6 consecutive amino acids comprise PLNGAV (SEQ ID NO: 3680);
   (iii) the 7 consecutive amino acids comprise PLNGAVH (SEQ ID NO: 3681);
   (iv) the 8 consecutive amino acids comprise PLNGAVHL (SEQ ID NO: 3682); or
   (v) the 9 consecutive amino acids comprise PLNGAVHLY (SEQ ID NO: 3648),
   wherein the AAV capsid variant comprises: (a) a VP1 protein comprising the amino acid sequence of SEQ ID NO: 138 or SEQ ID NO: 5, 8, or 3636; (b) a VP2 protein comprising the amino acid sequence of positions 138-736 of SEQ ID NO: 138 or positions 138-743 of SEQ ID NO: 5, 8, or 3636; (c) a VP3 protein comprising the amino acid sequence of positions 203-736 of SEQ ID NO: 138 or positions 203-743 of SEQ ID NO: 5, 8, or 3636; or (d) an amino acid sequence with at least 90% (e.g., at least about 95, 96, 97, 98, or 99%) sequence identity to any of the amino acid sequences in (a)-(c).
294. The isolated AAV particle of any one of embodiments 1-293, wherein the AAV capsid variant comprises an amino acid sequence comprising at least 5, 6, 7, 8, or 9 consecutive amino acids from the amino acid sequence of PLNGAVHLY (SEQ ID NO: 3648), wherein:
   (i) the 5 consecutive amino acids comprise PLNGA (SEQ ID NO: 3679);
   (ii) the 6 consecutive amino acids comprise PLNGAV (SEQ ID NO: 3680);
   (iii) the 7 consecutive amino acids comprise PLNGAVH (SEQ ID NO: 3681);
   (iv) the 8 consecutive amino acids comprise PLNGAVHL (SEQ ID NO: 3682); or
   (v) the 9 consecutive amino acids comprise PLNGAVHLY (SEQ ID NO: 3648),
   wherein the AAV capsid variant comprises the amino acid sequence of any one of SEQ ID NOs: 5, 8, or 3636 or an amino acid sequence with at least 90% (e.g., at least about 95, 96, 97, 98, or 99%) sequence identity to any one of SEQ ID NOs: 5, 8, or 3636.
295. The isolated AAV particle of any one of embodiments 1-294, wherein the AAV capsid variant comprises one or two, but no more than three different amino acids (e.g., substitutions, e.g., conservative substitutions) relative to the amino acid sequence of PLNGAVHLY (SEQ ID NO: 3648), wherein the AAV capsid variant comprises:
   (a) a VP1 protein comprising the amino acid sequence of SEQ ID NO: 5, 8, 138, or 3636;
   (b) a VP2 protein comprising the amino acid sequence of positions 138-736 of SEQ ID NO: 138 or positions 138-743 of SEQ ID NO: 5, 8, or 3636;
   (c) a VP3 protein comprising the amino acid sequence of positions 203-736 of SEQ ID NO: 138 or positions 203-743 of SEQ ID NO: 5, 8, or 3636; or
   (d) an amino acid sequence with at least 90% (e.g., at least about 95, 96, 97, 98, or 99%) sequence identity to any of the amino acid sequences in (a)-(c).
296. The isolated AAV particle of any one of embodiments 1-295, wherein the AAV capsid variant comprises one or two, but no more than three different amino acids (e.g., substitutions, e.g., conservative substitutions) relative to the amino acid sequence of PLNGAVHLY (SEQ ID NO: 3648), wherein the AAV capsid variant comprises the amino acid sequence of any one of SEQ ID NOs: 5, 8, 138, or 3636 or an amino acid sequence with at least 90% (e.g., at least about 95, 96, 97, 98, or 99%) sequence identity to any one of SEQ ID NOs: 5, 8, 138, or 3636.
297. The isolated AAV particle of any one of embodiments 293-296, wherein the amino acid sequence is present immediately subsequent to position 586, numbered according to any one of SEQ ID NO: 5, 8, 138, or 3636, optionally wherein the amino acid replaces positions 587 and 588 (e.g., A587 and Q588), numbered according to SEQ ID NO: 138.
298. The isolated AAV particle of any one of embodiments 293-297, wherein the amino acid sequence corresponds to positions 587-595 of SEQ ID NO: 5, 8, or 3636.
299. The isolated AAV particle of any one of embodiments 1-298, wherein the AAV capsid variant comprises one or two, but no more than three different amino acids (e.g., substitutions, e.g. conservative substitutions) relative to the amino acid sequence of PLNGAVHLYAQAQLSPVKN (SEQ ID NO: 566), wherein the AAV capsid variant comprises:
   (a) a VP1 protein comprising the amino acid sequence of SEQ ID NO: 5, 8, 138, or 3636;
   (b) a VP2 protein comprising the amino acid sequence of positions 138-736 of SEQ ID NO: 138 or positions 138-743 of SEQ ID NO: 5, 8, or 3636;
   (c) a VP3 protein comprising the amino acid sequence of positions 203-736 of SEQ ID NO: 138 or positions 203-743 of SEQ ID NO: 5, 8, or 3636; or
   (d) an amino acid sequence with at least 90% (e.g., at least about 95, 96, 97, 98, or 99%) sequence identity to any of the amino acid sequences in (a)-(c).
300. The isolated AAV particle of any one of embodiments 1-299, wherein the AAV capsid variant comprises one or two, but no more than three different amino acids (e.g., substitutions, e.g., conservative substitutions) relative to the amino acid sequence of PLNGAVHLYAQAQLSPVKN (SEQ ID NO: 566), wherein the AAV capsid variant comprises the amino acid sequence of any one of SEQ ID NOs: 5, 8, 138, or 3636 or an amino acid sequence with at least 90% (e.g., at least about 95, 96, 97, 98, or 99%) sequence identity to any one of SEQ ID NOs: 5, 8, 138, or 3636.
301. The isolated AAV particle of any one of embodiments 1-298, wherein the AAV capsid variant comprises one or two, but no more than three different amino acids (e.g., substitutions, e.g., conservative substitutions) relative to the amino acid sequence of PLNGAVHLYAQAQTGWVPN (SEQ ID NO: 314), wherein the AAV capsid variant comprises:
   (a) a VP1 protein comprising the amino acid sequence of SEQ ID NO: 5, 8, 138, or 3636;
   (b) a VP2 protein comprising the amino acid sequence of positions 138-736 of SEQ ID NO: 138 or positions 138-743 of SEQ ID NO: 5, 8, or 3636;
   (c) a VP3 protein comprising the amino acid sequence of positions 203-736 of SEQ ID NO: 138 or positions 203-743 of SEQ ID NO: 5, 8, or 3636; or
   (d) an amino acid sequence with at least 90% (e.g., at least about 95, 96, 97, 98, or 99%) sequence identity to any of the amino acid sequences in (a)-(c).
302. The isolated AAV particle of any one of embodiments 1-298 or 301, wherein the AAV capsid variant comprises one or two, but no more than three different amino acids (e.g., substitutions, e.g., conservative substitutions) relative to the amino acid sequence of PLNGAVHLYAQAQTGWVPN (SEQ ID NO: 314), wherein the AAV capsid variant comprises the amino acid sequence of any one of SEQ ID NOs: 5, 8, 138, or 3636 or an amino acid sequence with at least 90% (e.g., at least about 95, 96, 97, 98, or 99%) sequence identity to any one of SEQ ID NOs: 5, 8, 138, or 3636.
303. The isolated AAV particle of any one of embodiments 299-302, wherein the amino acid sequence is present immediately subsequent to position 586, numbered according to any one of SEQ ID NO: 5, 8, 138, or 3636, optionally wherein the amino acid replaces positions 587-598, numbered according to SEQ ID NO: 138.
304. The isolated AAV particle of any one of embodiments 299-303, wherein the amino acid sequence corresponds to positions 587-605 of SEQ ID NO: 5 or 8.
305. The isolated AAV particle of any one of the preceding embodiments, wherein the amino acid sequence is present in loop VIII.
306. The isolated AAV particle of any one of embodiments 3-5 or 271-305, wherein the amino acid sequence is present immediately subsequent to position 586, 587, 588, 589, 590, 591, 592, 593, 594, or 595, relative to a reference sequence numbered according to the amino acid sequence of SEQ ID NO: 138.
307. The isolated AAV particle of any one of embodiments 271-306, wherein the amino acid sequence is present immediately subsequent to position 586, relative to a reference sequence numbered according to the amino acid sequence of SEQ ID NO: 138.
308. The isolated AAV particle of any one of embodiments 271-307, wherein the amino acid sequence replaces positions 587 and 588 (e.g., A587 and Q588), numbered according to SEQ ID NO: 138.
309. The isolated AAV particle of any one of embodiments 271-306, wherein the amino acid sequence is present immediately subsequent to position 588, relative to a reference sequence numbered according to the amino acid sequence of SEQ ID NO: 138.
310. The isolated AAV particle of any one of embodiments 271-306, wherein the amino acid sequence is present immediately subsequent to position 592, relative to a reference sequence numbered according to the amino acid sequence of SEQ ID NO: 138.
311. The isolated AAV particle of any one of embodiments 271-306, wherein the amino acid sequence is present immediately subsequent to position 595, relative to a reference sequence numbered according to the amino acid sequence of SEQ ID NO: 138.
312. The isolated AAV particle of any one of embodiments 271-311, wherein the AAV capsid variant further comprises:
   (i) one, two, three, or all of an amino acid other than A at position 589, an amino acid other than Q at position 590, an amino acid other than A at position 591, and/or an amino acid other than Q at position 592, relative to a reference sequence numbered according to the amino acid sequence of SEQ ID NO: 138;
   (ii) one, two, or all of an amino acid other than T at position 593, an amino acid other than G at position 594, and/or an amino acid other than W at position 595, relative to a reference sequence numbered according to the amino acid sequence of SEQ ID NO: 138
   (iii) one, two, or all of an amino acid other than V at position 596, an amino acid other than Q at position 597, and/or an amino acid other than N at position 598, relative to a reference sequence numbered according to the amino acid sequence of SEQ ID NO: 138.
313. The isolated AAV particle of any one of the preceding embodiments, wherein the AAV capsid variant further comprises an amino acid other than A at position 587 and an amino acid other than Q at position 588, numbered according to the amino acid sequence of SEQ ID NO: 138.
314. The isolated AAV particle of any one of embodiments 271-313, wherein the AAV capsid variant comprises the amino acid P at position 587 the amino acid L at position 588, and the amino acid sequence NGAVHLY (SEQ ID NO: 3689), which is present immediately subsequent to position 588, corresponding to or numbered according to SEQ ID NO: 5, 8, 138, or 3636.
315. An isolated, e.g., recombinant, AAV particle comprising an AAV capsid variant and a nucleic acid comprising a transgene encoding a transgene encoding an antibody molecule which binds to tau, wherein the AAV capsid variant comprises the amino acid P at position 587 the amino acid L at position 588, and the amino acid sequence NGAVHLY (SEQ ID NO: 3689) present immediately subsequent to position 588, corresponding to or numbered according to SEQ ID NO: 5, 8, 138, or 3636.
316. The isolated AAV particle of any one of embodiments 271-315, wherein the AAV capsid variant further comprises:
   (i) one, two, or all of an amino acid other than T at position 593, an amino acid other than G at position 594, and/or an amino acid other than W at position 595, numbered according to the amino acid sequence of SEQ ID NO: 138; or
   (ii) one, two, or all of an amino acid other than T at position 600, an amino acid other than G at position 601, and/or an amino acid other than W at position 602, numbered according to the amino acid sequence of SEQ ID NO: 5, 8, or 3636.
317. The isolated AAV particle of any one of embodiments 271-316, wherein the AAV capsid variant further comprises:
   (i) an amino acid other than T at position 593, an amino acid other than G at position 594, and an amino acid other than W at position 595, numbered according to the amino acid sequence of SEQ ID NO: 138; or
   (ii) an amino acid other than T at position 600, an amino acid other than G at position 601, and an amino acid other than W at position 602, numbered according to the amino acid sequence of SEQ ID NO: 5, 8, 3636.
318. The isolated AAV particle of any one of embodiments 271-317, wherein the AAV capsid variant further comprises:
   (i) one, two, or all of the amino acid L at position 593, the amino acid S at position 594, and/or the amino acid P at position 595, numbered according to the amino acid sequence of SEQ ID NO: 138;
   (ii) one, two, or all of the amino acid L at position 600, the amino acid S at position 601, and/or the amino acid P at position 602, numbered according to the amino acid sequence of SEQ ID NO: 5, 8, or 3636.
319. The isolated AAV particle of any one of embodiments 271-318, wherein the AAV capsid variant further comprises:
   (i) the amino acid L at position 593, the amino acid S at position 594, and the amino acid P at position 595, numbered according to the amino acid sequence of SEQ ID NO: 138; or
   (ii) the amino acid L at position 600, the amino acid S at position 601, and the amino acid P at position 602, numbered according to the amino acid sequence of SEQ ID NO: 5, 8, or 3636.
320. The isolated AAV particle of any one of embodiments 271-319, wherein the AAV capsid variant further comprises:
   (i) one, two, or all of an amino acid other than V at position 596, an amino acid other than Q at position 597, and/or an amino acid other than N at position 598, numbered according to the amino acid sequence of SEQ ID NO: 138;
   (ii) one, two, or all of an amino acid other than V at position 603, an amino acid other than Q at position 604, and/or an amino acid other than N at position 605, numbered according to the amino acid sequence of SEQ ID NO: 5, 8, 3636.
321. The isolated AAV particle of any one of embodiments 271-320, wherein the AAV capsid variant further comprises an amino acid other than Q at position 597 numbered according to the amino acid sequence of SEQ ID NO: 138 or at position 604 numbered according to SEQ ID NO: 5, 8, or 3636.
322. The isolated AAV particle of any one of embodiments 271-321, wherein the AAV capsid variant further comprises the amino acid P at position 597 numbered according to the amino acid sequence of SEQ ID NO: 138 or at position 604 numbered according to SEQ ID NO: 5, 8, or 3636.
323. The isolated AAV particle of any one of embodiments 271-321, wherein the AAV capsid variant further comprises the amino acid K at position 597 numbered according to the amino acid sequence of SEQ ID NO: 138 or at position 604 numbered according to SEQ ID NO: 5, 8, or 3636.
324. The isolated AAV particle of any one of embodiments 271-321, wherein the AAV capsid variant further comprises the amino acid E or H at position 597 numbered according to the amino acid sequence of SEQ ID NO: 138 or at position 604 numbered according to SEQ ID NO: 5, 8, or 3636.
325. The isolated AAV particle of any one of embodiments 271-321 or 323, wherein the AAV capsid variant further comprises the amino acid L at position 593, the amino acid S at position 594, the amino acid P at position 595, and the amino acid K at position 597, numbered according to the amino acid sequence of SEQ ID NO: 138.
326. The isolated AAV particle of any one of embodiments 271-321, 323, or 325, wherein the AAV capsid variant further comprises the amino acid L at position 600, the amino acid S at position 601, the amino acid P at position 602, and the amino acid K at position 604, corresponding to or numbered according to the amino acid sequence of SEQ ID NO: 8 or 3636.
327. The isolated AAV particle of any one of embodiments 271-321, 323, 325, or 326, wherein the AAV capsid variant comprises:
   (i) the amino acid sequence of PLNGAVHLY (SEQ ID NO: 3648), optionally where the amino acid sequence is present immediately subsequent to position 586 and replaces positions 587 and 588 (e.g., A587 and Q 588), numbered according to SEQ ID NO: 138; and
   (ii) the amino acid L at position 593, the amino acid S at position 594, the amino acid P at position 595, and the amino acid K at position 597, numbered according to the amino acid sequence of SEQ ID NO: 138.
328. The isolated AAV particle of any one of embodiments 271-321, 323, or 325-327, wherein the AAV capsid variant comprises:
   (i) the amino acid P at position 587, the amino acid L at position 588, and the amino acid sequence NGAVHLY (SEQ ID NO: 3689), which is present immediately subsequent to position 588, numbered according to SEQ ID NO: 138; and
   (ii) the amino acid L at position 593, the amino acid S at position 594, the amino acid P at position 595, and the amino acid K at position 597, numbered according to the amino acid sequence of SEQ ID NO: 138.
329. The isolated AAV particle of any one of embodiments 271-321, 323, or 325-328, wherein the AAV capsid variant comprises:
   (i) the amino acid P at position 587, the amino acid L at position 588, and the amino acid sequence NGAVHLY (SEQ ID NO: 3689), which is present immediately subsequent to position 588, corresponding to or numbered according to SEQ ID NO: 8 or 3636; and
   (ii) the amino acid L at position 600, the amino acid S at position 601, the amino acid P at position 602, and the amino acid K at position 604, corresponding to or numbered according to the amino acid sequence of SEQ ID NO: 8 or 3636.
330. The isolated AAV particle of any one of embodiments 271-322, wherein the AAV capsid variant further comprises the amino acid P at position 597, numbered according to the amino acid sequence of SEQ ID NO: 138.
331. The isolated AAV particle of any one of embodiments 271-322 or 330, wherein the AAV capsid variant further comprises the amino acid P at position 604, corresponding to or numbered according to the amino acid sequence of SEQ ID NO: 5 or 3636.
332. The isolated AAV particle of any one of embodiments 271-322, 330, or 331, wherein the AAV capsid variant comprises:
   (i) the amino acid sequence of PLNGAVHLY (SEQ ID NO: 3648), optionally where the amino acid sequence is present immediately subsequent to position 586 and replaces positions 587 and 588 (e.g., A587 and Q 588), numbered according to SEQ ID NO: 138; and
   (ii) the amino acid P at position 597, numbered according to the amino acid sequence of SEQ ID NO: 138.
333. The isolated AAV particle of any one of embodiments 271-322 or 330-332, wherein the AAV capsid variant comprises:
   (i) the amino acid P at position 587, the amino acid L at position 588, and the amino acid sequence NGAVHLY (SEQ ID NO: 3689), which is present immediately subsequent to position 588, numbered according to SEQ ID NO: 138; and
   (ii) the amino acid P at position 597, numbered according to the amino acid sequence of SEQ ID NO: 138.
334. The isolated AAV particle of any one of embodiments 271-322 or 330-333, wherein the AAV capsid variant comprises:
   (i) the amino acid P at position 587, the amino acid L at position 588, and the amino acid sequence NGAVHLY (SEQ ID NO: 3689), which is present immediately subsequent to position 588, corresponding to or numbered according to SEQ ID NO: 5 or 3636; and
   (ii) the amino acid P at position 604, corresponding to or numbered according to the amino acid sequence of SEQ ID NO: 5 or 3636.
335. An isolated, e.g., recombinant, AAV particle comprising an AAV capsid variant and a nucleic acid comprising a transgene encoding a transgene encoding an antibody molecule which binds to tau, wherein the AAV capsid variant comprises:
   (i) the amino acid sequence of PLNGAVHLY (SEQ ID NO: 3648), optionally where the amino acid sequence is present immediately subsequent to position 586 and replaces positions 587 and 588 (e.g., A587 and Q 588), numbered according to SEQ ID NO: 138; and
   (ii) the amino acid L at position 593, the amino acid S at position 594, the amino acid P at position 595, and the amino acid K at position 597, numbered according to the amino acid sequence of SEQ ID NO: 138.
336. An isolated, e.g., recombinant, AAV particle comprising an AAV capsid variant and a nucleic acid comprising a transgene encoding a transgene encoding an antibody molecule which binds to tau, wherein the AAV capsid variant comprises:
   (i) the amino acid P at position 587, the amino acid L at position 588, and the amino acid sequence NGAVHLY (SEQ ID NO: 3689), which is present immediately subsequent to position 588, numbered according to SEQ ID NO: 138; and
   (ii) the amino acid L at position 593, the amino acid S at position 594, the amino acid P at position 595, and the amino acid K at position 597, numbered according to the amino acid sequence of SEQ ID NO: 138.
337. An isolated, e.g., recombinant, AAV particle comprising an AAV capsid variant and a nucleic acid comprising a transgene encoding a transgene encoding an antibody molecule which binds to tau, wherein the AAV capsid variant comprises:
   (i) the amino acid P at position 587, the amino acid L at position 588, and the amino acid sequence NGAVHLY (SEQ ID NO: 3689), which is present immediately subsequent to position 588, corresponding to or numbered according to SEQ ID NO: 8 or 3636; and
   (ii) the amino acid L at position 600, the amino acid S at position 601, the amino acid P at position 602, and the amino acid K at position 604, corresponding to or numbered according to the amino acid sequence of SEQ ID NO: 8 or 3636.
338. An isolated, e.g., recombinant, AAV particle comprising an AAV capsid variant and a nucleic acid comprising a transgene encoding a transgene encoding an antibody molecule which binds to tau, wherein the AAV capsid variant comprises:
   (i) the amino acid sequence of PLNGAVHLY (SEQ ID NO: 3648), optionally where the amino acid sequence is present immediately subsequent to position 586 and replaces positions 587 and 588 (e.g., A587 and Q 588), numbered according to SEQ ID NO: 138; and
   (ii) the amino acid P at position 597, numbered according to the amino acid sequence of SEQ ID NO: 138.
339. An isolated, e.g., recombinant, AAV particle comprising an AAV capsid variant and a nucleic acid comprising a transgene encoding a transgene encoding an antibody molecule which binds to tau, wherein the AAV capsid variant comprises:
   (i) the amino acid P at position 587, the amino acid L at position 588, and the amino acid sequence NGAVHLY (SEQ ID NO: 3689), which is present immediately subsequent to position 588, numbered according to SEQ ID NO: 138; and
   (ii) the amino acid P at position 597, numbered according to the amino acid sequence of SEQ ID NO: 138.
340. An isolated, e.g., recombinant, AAV particle comprising an AAV capsid variant and a nucleic acid comprising a transgene encoding a transgene encoding an antibody molecule which binds to tau, wherein the AAV capsid variant comprises:
   (i) the amino acid P at position 587, the amino acid L at position 588, and the amino acid sequence NGAVHLY (SEQ ID NO: 3689), which is present immediately subsequent to position 588, corresponding to or numbered according to SEQ ID NO: 5 or 3636; and
   (ii) the amino acid P at position 604, corresponding to or numbered according to the amino acid sequence of SEQ ID NO: 5 or 3636.
341. The isolated AAV particle of any one of the preceding embodiments, wherein the AAV capsid variant further comprises:
   (i) a modification, e.g., an insertion, substitution (e.g., conservative substitution), and/or deletion, in loop I, II, IV, and/or VI; and/or
   (ii) a substitution at position K449, e.g., a K449R substitution, numbered according to SEQ ID NO: 138.
342. The isolated AAV particle of any one of the preceding embodiments, wherein the AAV capsid variant comprises an amino acid sequence comprising at least one, two or three modifications, e.g., substitutions (e.g., conservative substitutions), but not more than 30, 20 or 10 modifications, e.g., substitutions (e.g., conservative substitutions), relative to the amino acid sequence of SEQ ID NO: 138.
343. The isolated AAV particle of any one of the preceding embodiments, wherein the AAV capsid variant comprises an amino acid sequence comprising at least one, two or three, but no more than 30, 20 or 10 different amino acids relative to the amino acid sequence of SEQ ID NO: 138.
344. The isolated AAV particle of any one of the preceding embodiments, wherein the AAV capsid variant comprises the amino acid sequence of SEQ ID NO: 138, or an amino acid sequence with at least 80% (e.g., at least about 85, 90, 95, 96, 97, 98, or 99%) sequence identity thereto.
345. The isolated AAV particle of any one of the preceding embodiments, wherein the AAV capsid variant comprises the amino acid sequence of SEQ ID NO: 138.
346. The isolated AAV particle of any one of the preceding embodiments, wherein the AAV capsid variant comprises an amino acid sequence encoded by the nucleotide sequence of SEQ ID NO: 137, or a sequence with at least 80% (e.g., at least about 85, 90, 95, 96, 97, 98, or 99%) sequence identity thereto.
347. The isolated AAV particle of any one of the preceding embodiments, wherein the nucleotide sequence encoding the capsid variant comprises the nucleotide sequence of SEQ ID NO: 137, or a sequence with at least 80% (e.g., at least about 85, 90, 95, 96, 97, 98, or 99%) sequence identity thereto.
348. The isolated AAV particle of any one of the preceding embodiments, wherein the AAV capsid variant comprises a VP1 protein, a VP2 protein, a VP3 protein, or a combination thereof.
349. The isolated AAV particle of any one of embodiments 1-348, wherein the AAV capsid variant comprises the amino acid sequence corresponding to positions 138-743, e.g., a VP2, of SEQ ID NO: 5, 8, or 3636, or a sequence with at least 80% (e.g., at least about 85, 90, 95, 96, 97, 98, or 99%) sequence identity thereto.
350. The isolated AAV particle of any one of embodiments 1-349, wherein the AAV capsid variant comprises the amino acid sequence corresponding to positions 203-743, e.g., a VP3, of SEQ ID NO: 5, 8, or 3636, or a sequence with at least 80% (e.g., at least about 85, 90, 95, 96, 97, 98, or 99%) sequence identity thereto.
351. The isolated AAV particle of any one of embodiments 1-350, wherein the AAV capsid variant comprises the amino acid sequence corresponding to positions 138-736, e.g., a VP2, of SEQ ID NO: 138, or a sequence with at least 80% (e.g., at least about 85, 90, 95, 96, 97, 98, or 99%) sequence identity thereto.
352. The isolated AAV particle of any one of embodiments 1-351, wherein the AAV capsid variant comprises the amino acid sequence corresponding to positions 203-736, e.g., a VP3, of SEQ ID NO: 138, or a sequence with at least 80% (e.g., at least about 85, 90, 95, 96, 97, 98, or 99%) sequence identity thereto.
353. An isolated, e.g., recombinant, AAV particle comprising an AAV capsid variant and a nucleic acid comprising a transgene encoding a transgene encoding an antibody molecule which binds to tau, wherein the AAV capsid variant comprises the amino acid sequence of SEQ ID NO: 5.
354. The isolated AAV particle of embodiment 353, wherein the nucleotide sequence encoding the AAV capsid variant comprises the nucleotide sequence of SEQ ID NO: 4.
355. An isolated, e.g., recombinant, AAV particle comprising an AAV capsid variant and a nucleic acid comprising a transgene encoding a transgene encoding an antibody molecule which binds to tau, wherein the AAV capsid variant comprises the amino acid sequence of SEQ ID NO: 8.
356. The isolated AAV particle of embodiment 355, wherein the nucleotide sequence encoding the AAV capsid variant comprises the nucleotide sequence of SEQ ID NO: 7.
357. An isolated, e.g., recombinant, AAV particle comprising an AAV capsid variant and a nucleic acid comprising a transgene encoding a transgene encoding an antibody molecule which binds to tau, wherein the AAV capsid variant comprises the amino acid sequence of SEQ ID NO: 3636.
358. The isolated AAV particle of embodiment 357, wherein the nucleotide sequence encoding the AAV capsid variant comprises the nucleotide sequence of SEQ ID NO: 3623.
359. The isolated AAV particle of any one of the preceding embodiments, wherein the AAV capsid variant does not comprise:
   (i) the amino acid sequence of TLAVPFK (SEQ ID NO: 1262) present immediately subsequent to position 588, numbered according to SEQ ID NO: 138;
   (ii) an amino acid sequence present immediately subsequent to position 586 to 599, e.g., 586 to 594, 587 to 595, 588 to 596, 589 to 597, 590 to 598 numbered relative to SEQ ID NO: 138, having at least 5 consecutive amino acids corresponding to positions 586 to 594 numbered relative to SEQ ID NO: 138, of any the amino acid sequences provided in Table 1 of WO2020223276, the contents of which are hereby incorporated by reference in their entirety; or
   (iii) an amino acid sequence present immediately subsequent to position 586 to 599, e.g., 586 to 594, 587 to 595, 588 to 596, 589 to 597, 590 to 598 numbered relative to SEQ ID NO: 138, having at least 5 consecutive amino acids corresponding to positions 586 to 594 numbered relative to SEQ ID NO: 138, of any SEQ ID NOs: 1, 12, 13, or 138.
360. The isolated AAV particle of any one of the preceding embodiments, wherein the AAV capsid variant has an increased tropism for a CNS cell or tissue, e.g., a brain cell, brain tissue, spinal cord cell, or spinal cord tissue, relative to the tropism of a reference sequence comprising the amino acid sequence of SEQ ID NO: 138.
361. The isolated AAV particle of any one of embodiments 15-360, wherein the AAV capsid variant has an increased tropism for a CNS cell or tissue, e.g., a brain cell, brain tissue, spinal cord cell, or spinal cord tissue, relative to the tropism of a reference sequence comprising the amino acid sequence of SEQ ID NO: 3636.
362. The isolated AAV particle of any one of embodiments 15-361, wherein the AAV capsid variant is enriched at least about 1.1, 1.2, 1.3, 1.4, 1.5, 1.6, 1.7, 1.8, 1.9, 2, 2.5, 3, 3.5, 4, 4.5, 5, 5.5, or 6-fold, in the brain compared to a reference sequence of SEQ ID NO: 3636, e.g., when measured by an assay as described in Example 1.
363. The isolated AAV particle of any one of embodiments 72, 75, 76, 271, 278, 281, 286, 287, 292, or 305-362, wherein the AAV capsid variant results in greater than 1, 2, 5, 10, 20, 30, 40, 50, or 100 reads per sample, e.g., when analyzed by an NGS sequencing assay, e.g., as described in Example 1.
364. The isolated AAV particle of any one of embodiments 15-363, wherein the AAV capsid variant is enriched in the brain of at least two to three species, e.g., a non-human primate and rodent (e.g., mouse), e.g., as compared to a reference sequence of SEQ ID NO: 138.
365. The isolated AAV particle of embodiment 364, wherein the at least two to three species are Macaca fascicularis, Chlorocebus sabaeus, Callithrix jacchus, and/or mouse (e.g., BALB/c mice).
366. The isolated AAV particle of any one of the preceding embodiments, wherein the AAV capsid variant further comprises a modification, e.g., substitution (e.g., conservative substitution), insertion, or deletion, that results in one, two, three or all of: (1) reduced tropism in the liver; (2) de-targeted expression in the liver; (3) reduced activity in the liver; and/or (4) reduced binding to galactose.
367. The AAV capsid variant of any one of the preceding embodiments, wherein the AAV capsid variant further comprises:
   (i) a modification e.g., substitution (e.g., conservative substitution), insertion, or deletion, at position N470 (e.g., N470A), D271 (e.g., D271A), N272 (e.g., N297A), Y446 (e.g., Y446A), N498 (e.g., N498Y or N498I), W503 (e.g., W530R or W530A), L620 (e.g., L620F), or a combination thereof, relative to a reference sequence numbered according to SEQ ID NO: 138; or
   (ii) one, two, three, four, five or all of an amino acid other than N at position 470 (e.g., A), an amino acid other than D at position 271 (e.g., A), an amino acid other than N at position 272 (e.g., A), an amino acid other than Y at position 446 (e.g., A), and amino acid other than N at position 498 (e.g., Y or I), and amino acid other than W at position 503 (e.g., R or A), and amino acid other than L at position 620 (e.g., F), relative to a reference sequence numbered according to SEQ ID NO: 138.
368. The isolated AAV particle of any one of the preceding embodiments, wherein the antibody molecule binds to the N-terminal region or an epitope within the N-terminal region of a tau protein (e.g., human tau).
369. The isolated AAV particle of embodiment 368, wherein the antibody molecule binds to all or a portion of (e.g., one or more residues within) amino acid residues (a) 9-18, (b) 15-25, (c) 25-30, and/or (d) 15-30 of a tau protein, numbered according to SEQ ID NO: 9200.
370. The isolated AAV particle of any one of embodiments 1-367, wherein the antibody molecule binds to the mid-domain region or an epitope within the mid-domain region of a tau protein (e.g., human tau).
371. The isolated AAV particle of embodiment 370, wherein the antibody molecule binds to all or a portion of (e.g., one or more residues within) amino acid residues (a) 125-131, (b) 204-222, (c) 234-246, (d) 234-259, and/or (e) 235-246, numbered according to SEQ ID NO: 9200.
372. The isolated AAV particle of embodiment 371, wherein the antibody molecule binds to an epitope which includes one or more of pT181, pS199, pS202, pT205, pT212, pS214, pT217, pT231, pS234, pS235, pS258, pS259, pS396, pS404, and pS409, numbered according to SEQ ID NO: 9200.
373. The isolated AAV particle of embodiment 372, wherein the antibody molecule binds to an epitope which includes (a) pS202 and pT205, (b) pT212 and pS214, (c) pT212 and pT217, (c) pT217 and pT231, (d) pT231 and pT234 (or pT235), (e) pS234 and pS259 (or pS258), and (f) pS396 and pS404, numbered according to SEQ ID NO: 9200.
374. The isolated AAV particle of any one of embodiments 1-367, wherein the antibody molecule binds to the C-terminal region or an epitope within the C-terminal region of a tau protein (e.g., human tau).
375. The isolated AAV particle of embodiment 374, wherein the antibody molecule binds to all or a portion of (e.g., one or more residues within) amino acid residues (a) 387-408 and/or (b) 409-436, numbered according to SEQ ID NO: 9200.
376. The isolated AAV particle of embodiment 374, wherein the antibody molecule binds to an epitope which includes pS409, numbered according to SEQ ID NO: 9200.
377. The isolated AAV particle of any one of embodiments 1-367, wherein the antibody molecule binds to the microtubule-binding domain or an epitope within the microtubule-binding domain of a tau protein (e.g., human tau).
378. The isolated AAV particle of any one of embodiments 1-367, wherein the antibody molecule binds to the proline rich domain or an epitope within the proline rich domain of a tau protein (e.g., human tau).
379. The isolated AAV particle of any one of embodiments 1-367, wherein the antibody molecule binds to a region that overlaps with the N-terminal domain and the mid-domain of a tau protein (e.g., human tau).
380. The isolated AAV particle of any one of embodiments 1-367, wherein the antibody molecule binds to a region that overlaps with the mid-domain and the microtubule-binding domain of a tau protein (e.g., human tau).
381. The isolated AAV particle of any one of embodiments 1-367, wherein the antibody molecule binds to a region that overlaps with the mid-domain and the C-terminal domain of a tau protein (e.g., human tau).
382. The isolated AAV particle of any one of embodiments 1-367, wherein the antibody molecule binds to all or a portion of (e.g., one or more residues within) amino acid residues (a) 32-49, (b) 55-76, (c) 57-72, (d) 159-194, (e) 175-191, (f) 185-200, (g) 219-247, (h) 223-238, (i) 381-426, (j) 383-400, (k) 409-436, and/or (1) 413-430, numbered according to SEQ ID NO: 9200.
383. The isolated AAV particle of embodiment 382, wherein the antibody molecule binds to all or a portion of (e.g., one or more residues within) amino acid residues (a) 55-76, (b) 159-194, (c) 219-247, and/or (d) 381-426, numbered according to SEQ ID NO: 9200.
384. The isolated AAV particle of embodiment 382, wherein the antibody molecule binds to all or a portion of (e.g., one or more residues within) amino acid residues (a) 57-72, (b) 175-191, (c) 223-238, and/or (d) 383-400, numbered according to SEQ ID NO: 9200.
385. The isolated AAV particle of embodiment 382 and 384, wherein the antibody molecule binds to all or a portion of (e.g., one or more residues within) amino acid residues 223-238, numbered according to SEQ ID NO: 9200.
386. The isolated AAV particle of embodiment 382, wherein the antibody molecule binds a conformational epitope which includes all or a portion of (e.g., one or more residues within) amino acid residues (a) 55-76, (b) 159-194, (c) 219-247, and/or (d) 381-426, numbered according to SEQ ID NO: 9200.
387. The isolated AAV particle of any one of embodiments 1-386, wherein the antibody molecule comprises:
   (i) a heavy chain variable region comprising the HCDR1, HCDR2, and HCDR3 sequences of any one of the anti-tau antibody molecules described herein (e.g., an antibody molecule listed in Tables 7-16); and/or
   (ii) a light chain variable region comprising the LCDR1, LCDR2, and LCDR3 sequences of any one of the anti-tau antibody molecules described herein (e.g., an antibody molecule listed in Tables 7-16).
388. The isolated AAV particle of any one of embodiments 1-387, wherein the antibody molecule comprises a heavy chain variable region comprising HCDR1, HCDR2, and HCDR3, and a light chain variable region comprising LCDR1, LCDR2, and LCDR3, of any one of the anti-tau antibody molecules described herein (e.g., an antibody molecule listed in Tables 7-16).
389. The isolated AAV particle of any one of embodiments 1-388, wherein the antibody molecule comprises:
   (i) a heavy chain variable region (VH) comprising the amino acid sequence of the VH of any one of the anti-tau antibody molecules described herein (e.g., the VH sequence of an antibody molecule listed in Tables 7-16), or an amino acid sequence having at least 80% (e.g., 85, 90, 95, 96, 97, 98, or 99%) sequence identity to the VH sequence of any one of the anti-tau antibody molecules described herein (e.g., the VH sequence of an antibody molecule listed in Tables 7-16), or an amino acid sequence having at least one, two or three modifications, but not more than 30, 20 or 10 modifications, to the amino acid sequence of the VH of any of the anti-tau antibody molecules described herein (e.g., the VH sequence of an antibody molecule listed in Tables 7-16); and/or
   (ii) a light chain variable region (VL) comprising the amino acid sequence of the VL of any one of the anti-tau antibody molecules described herein (e.g., the VL sequence of an antibody molecule listed in Tables 7-16), or an amino acid sequence having at least 80% (e.g., 85, 90, 95, 96, 97, 98, or 99%) sequence identity to the VL sequence of any one of the anti-tau antibody molecules described herein (e.g., the VL sequence of an antibody molecule listed in Tables 7-16), or an amino acid sequence having at least one, two or three modifications, but not more than 30, 20 or 10 modifications, to the amino acid sequence of the VL of any of the anti-tau antibody molecules described herein (e.g., the VL sequence of an antibody molecule listed in Tables 7-16).
390. The isolated AAV particle of any one of embodiments 1-389, wherein the antibody molecule comprises the VH region and the VL region of any one of the anti-tau antibody molecule described herein (e.g., the VH and VL region sequences of an antibody molecule listed in Tables 7-16).
391. The isolated AAV particle of any one of embodiments 1-390, wherein the antibody molecule comprises the HCDR1, HCDR2, and/or HCDR3 sequences, and/or LCDR1, LCDR2, and/or LCDR3 sequences of V0022, V0004, V0009, V0023, V0024, V0052, IPN002, AT8, PT3, UCB, PT76, PHF1, or C10.2.
392. The isolated AAV particle of any one of embodiments 1-391, wherein the antibody molecule comprises:
   (i) an HCDR1, an HCDR2, and an HCDR3 comprising the amino acid sequence of SEQ ID NOs: 1631, 1551, and 1552, respectively; and/or an LCDR1, an LCDR2, and an LCDR3 comprising the amino acid sequence of SEQ ID NOs: 1632, 1633, and 1566, respectively;
   (ii) an HCDR1, an HCDR2, and an HCDR3 comprising the amino acid sequence of SEQ ID NOs: 1634, 1635, and 1552, respectively; and/or an LCDR1, an LCDR2, and an LCDR3 comprising the amino acid sequence of SEQ ID NOs: 1636, 1633, and 1566, respectively; or
   (iii) an HDR1, an HCDR2, and an HCDR3 comprising the amino acid sequence of SEQ ID NOs: 1637, 1638, and 1557, respectively; and/or an LCDR1, an LCDR2, and an LCDR3 comprising the amino acid sequence of SEQ ID NOs: 1639, 1569, and 1566, respectively.
393. The isolated AAV particle of any one of embodiments 1-392, wherein the antibody molecule comprises:
   (i) an HCDR1, an HCDR2, and an HCDR3 comprising the amino acid sequence of SEQ ID NOs: 1550, 1551, and 1552, respectively; and/or an LCDR1, an LCDR2, and an LCDR3 comprising the amino acid sequence of SEQ ID NOs: 1564, 1565, and 1566, respectively;
   (ii) an HCDR1, an HCDR2, and an HCDR3 comprising the amino acid sequence of SEQ ID NO: 1553, 1554, and 1552, respectively; and/or an LCDR1, an LCDR2, and an LCDR3 comprising the amino acid sequence of SEQ ID NO: 1567, 1565, and 1566, respectively;
   (iii) an HCDR1, an HCDR2, and an HCDR3 comprising the amino acid sequence of SEQ ID NOs: 1555, 1556, and 1557, respectively; and/or an LCDR1, an LCDR2, and an LCDR3 comprising the amino acid sequence of SEQ ID NOs: 1568, 1569, and 1566, respectively,
   (iv) the HCDR1, HCDR2, and/or HCDR3 sequences, and/or LCDR1, LCDR2, and/or LCDR3 sequences of any one of (i)-(iii), with at most one, two, or three substitutions (e.g., conservative amino acid substitutions) in each CDR.
394. The isolated AAV particle of any one of embodiments 1-392, wherein the antibody molecule comprises:
   (i) an HCDR1, an HCDR2, and an HCDR3 comprising the amino acid sequence of SEQ ID NOs: 1575, 1551, and 1552, respectively; and/or an LCDR1, an LCDR2, and an LCDR3 comprising the amino acid sequence of SEQ ID NOs: 1585, 1565, and 1566, respectively;
   (ii) an HCDR1, an HCDR2, and an HCDR3 comprising the amino acid sequence of SEQ ID NOs: 1576, 1577, and 1552, respectively; and/or an LCDR1, an LCDR2, and an LCDR3 comprising the amino acid sequence of SEQ ID NOs: 1585, 1565, and 1566, respectively;
   (iii) an HCDR1, an HCDR2, and an HCDR3 comprising the amino acid sequence of SEQ ID NOs: 1578, 1579, and 1557, respectively; and/or an LCDR1, an LCDR2, and an LCDR3 comprising the amino acid sequence of SEQ ID NOs: 1586, 1569, and 1566, respectively;
   (iv) the HCDR1, HCDR2, and/or HCDR3 sequences, and/or LCDR1, LCDR2, and/or LCDR3 sequences of any one of (i)-(iii), with at most one, two, or three substitutions (e.g., conservative amino acid substitutions) in each CDR.
395. The isolated AAV particle of any one of embodiments 1-392, wherein the antibody molecule comprises:
   (i) an HCDR1, an HCDR2, and an HCDR3 comprising the amino acid sequence of SEQ ID NOs: 1593, 1551, and 1552, respectively; and/or an LCDR1, an LCDR2, and an LCDR3 comprising the amino acid sequence of SEQ ID NOs: 1601, 1602, and 1566, respectively;
   (ii) an HCDR1, an HCDR2, and an HCDR3 comprising the amino acid sequence of SEQ ID NO: 1594, 1595, and 1552, respectively; and/or an LCDR1, an LCDR2, and an LCDR3 comprising the amino acid sequence of SEQ ID NO: 1601, 1602, and 1566, respectively;
   (iii) an HCDR1, an HCDR2, and an HCDR3 comprising the amino acid sequence of SEQ ID NOs: 1596, 1556, and 1557, respectively; and/or an LCDR1, an LCDR2, and an LCDR3 comprising the amino acid sequence of SEQ ID NOs: 1603, 1569, and 1566, respectively;
   (iv) the HCDR1, HCDR2, and/or HCDR3 sequences, and/or LCDR1, LCDR2, and/or LCDR3 sequences of any one of (i)-(iii), with at most one, two, or three substitutions (e.g., conservative amino acid substitutions) in each CDR.
396. The isolated AAV particle of any one of embodiments 1-391, wherein the antibody molecule comprises:
   (i) an HCDR1, an HCDR2, and an HCDR3 comprising the amino acid sequence of SEQ ID NOs: 1608, 1609, and 1610, respectively; and/or an LCDR1, an LCDR2, and an LCDR3 comprising the amino acid sequence of SEQ ID NOs: 1621, 1622, and 1623, respectively;
   (ii) an HCDR1, an HCDR2, and an HCDR3 comprising the amino acid sequence of SEQ ID NO: 1611, 1612, and 1610, respectively; and/or an LCDR1, an LCDR2, and an LCDR3 comprising the amino acid sequence of SEQ ID NO: 1621, 1622, and 1623, respectively;
   (iii) an HCDR1, an HCDR2, and an HCDR3 comprising the amino acid sequence of SEQ ID NOs: 1613, 1614, and 1615, respectively; and/or an LCDR1, an LCDR2, and an LCDR3 comprising the amino acid sequence of SEQ ID NOs: 1624, 1625, and 1623, respectively;
   (iv) the HCDR1, HCDR2, and/or HCDR3 sequences, and/or LCDR1, LCDR2, and/or LCDR3 sequences of any one of (i)-(iii), with at most one, two, or three substitutions (e.g., conservative amino acid substitutions) in each CDR.
397. The isolated AAV particle of any one of embodiments 1-391, wherein the antibody molecule comprises:
   (i) an HCDR1, an HCDR2, and an HCDR3 comprising the amino acid sequence of SEQ ID NOs: 1525, 1526, and 1527, respectively; and/or an LCDR1, an LCDR2, and an LCDR3 comprising the amino acid sequence of SEQ ID NOs: 1539, 1540, and 1541, respectively;
   (ii) an HCDR1, an HCDR2, and an HCDR3 comprising the amino acid sequence of SEQ ID NO: 1528, 1529, and 1527, respectively; and/or an LCDR1, an LCDR2, and an LCDR3 comprising the amino acid sequence of SEQ ID NO: 1539, 1540, and 1541, respectively;
   (iii) an HCDR1, an HCDR2, and an HCDR3 comprising the amino acid sequence of SEQ ID NOs: 1530, 1531, and 1532, respectively; and/or an LCDR1, an LCDR2, and an LCDR3 comprising the amino acid sequence of SEQ ID NOs: 1542, 1543, and 1541, respectively; or
   (iv) the HCDR1, HCDR2, and/or HCDR3 sequences, and/or LCDR1, LCDR2, and/or LCDR3 sequences of any one of (i)-(iii), with at most one, two, or three substitutions (e.g., conservative amino acid substitutions) in each CDR.
398. The isolated AAV particle of any one of embodiments 1-391, wherein the antibody molecule comprises:
   (i) an HCDR1, an HCDR2, and an HCDR3 comprising the amino acid sequence of SEQ ID NOs: 1500, 1501, and 1502, respectively; and/or an LCDR1, an LCDR2, and an LCDR3 comprising the amino acid sequence of SEQ ID NOs: 1514, 1515, and 1516, respectively;
   (ii) an HCDR1, an HCDR2, and an HCDR3 comprising the amino acid sequence of SEQ ID NO: 1503, 1504, and 1502, respectively; and/or an LCDR1, an LCDR2, and an LCDR3 comprising the amino acid sequence of SEQ ID NO: 1514, 1515, and 1516, respectively;
   (iii) an HCDR1, an HCDR2, and an HCDR3 comprising the amino acid sequence of SEQ ID NOs: 1505, 1506, and 1507, respectively; and/or an LCDR1, an LCDR2, and an LCDR3 comprising the amino acid sequence of SEQ ID NOs: 1517, 1518, and 1516, respectively; or
   (iv) the HCDR1, HCDR2, and/or HCDR3 sequences, and/or LCDR1, LCDR2, and/or LCDR3 sequences of any one of (i)-(iii), with at most one, two, or three substitutions (e.g., conservative amino acid substitutions) in each CDR.
399. The isolated AAV particle of any one of embodiments 1-393, wherein the antibody molecule comprises:
   (a) the VH and/or VL sequences of SEQ ID NOs: 1562 and 1573, respectively;
   (b) the VH and/or VL sequences of SEQ ID NOs: 1512 and 1523, respectively;
   (c) the VH and/or VL sequences of SEQ ID NOs: 1537 and 1548, respectively;
   (d) the VH and/or VL sequences of SEQ ID NOs: 1583 and 1591, respectively;
   (e) the VH and/or VL sequences of SEQ ID NOs: 1599 and 1606, respectively;
   (f) the VH and/or VL sequences of SEQ ID NOs: 1619 and 1629, respectively;
   (g) the VH and/or VL sequences of SEQ ID NOs: 1822 and 1941, respectively;
   (h) the VH and/or VL sequences of SEQ ID NOs: 1849 and 1970, respectively;
   (i) the VH and/or VL sequences of SEQ ID NOs: 1844 and 1964, respectively;
   (j) the VH and/or VL sequences of SEQ ID NOs: 1803 and 1919, respectively;
   (k) a VH sequence having at least 80% (e.g., 85, 90, 95, 96, 97, 98, or 99%) sequence identity to the VH sequence of any one of (a)-(j), and/or a VL sequence having at least 80% (e.g., 85, 90, 95, 96, 97, 98, or 99%) sequence identity to the VL sequence of any one of (a)-(j); or
   (l) a VH sequence having at least one, two, or three modifications, but not more than 30, 20, or 10 modifications, relative to the VH sequence of any one of (a)-(j), and/or a VL sequence having at least one, two, or three modifications, but not more than 30, 20, or 10 modifications, relative to the VL sequence of any one of (a)-(j).
400. The isolated AAV particle of embodiment 399, wherein the antibody molecule comprises a VH sequence having at least 80% (e.g., 85, 90, 95, 96, 97, 98, or 99%) sequence identity to the VH sequence of SEQ ID NO: 1562, and/or a VL sequence having at least 80% (e.g., 85, 90, 95, 96, 97, 98, or 99%) sequence identity to the VL sequence of SEQ ID NO: 1573, respectively.
401. The isolated AAV particle of embodiment 399, wherein the antibody molecule comprises a VH sequence having at least one, two, or three modifications, but not more than 30, 20, or 10 modifications, relative to the VH sequence of SEQ ID NO: 1562, and/or a VL sequence having at least one, two, or three modifications, but not more than 30, 20, or 10 modifications, relative to the VL sequence of SEQ ID NO: 1573.
402. The isolated AAV particle of embodiment 399, wherein the antibody molecule comprises the VH and VL sequences of SEQ ID NOs: 1562 and 1573, respectively.
403. The isolated AAV particle of embodiment 402, wherein the antibody molecule comprises a VH sequence encoded by a nucleotide sequence having at least 80% (e.g., 85, 90, 95, 96, 97, 98, or 99%) sequence identity to any one of SEQ ID NOs: 1563, 1513, 1538, 1584, 1600, or 1620 and/or a VL sequence encoded by a nucleotide sequence having at least 80% (e.g., 85, 90, 95, 96, 97, 98, or 99%) sequence identity to any one of SEQ ID NOs: 1574, 1524, 1549, 1592, 1607, or 1630.
404. The isolated AAV particle of embodiment 403, wherein the antibody molecule comprises VH and VL sequences encoded by nucleotide sequences set forth in (a) SEQ ID NOs: 1563 and 1574, respectively, (b) SEQ ID NOs: 1513 and 1524, respectively, (c) SEQ ID NOs: 1538 and 1549, respectively, (d) SEQ ID NOs: 1584 and 1592, respectively, (e) SEQ ID NOs: 1600 and 1607, respectively, (f) SEQ ID NOs: 1620 and 1630, respectively, or (g) a nucleotide sequence having at least 80% (e.g., 85, 90, 95, 96, 97, 98, or 99%) sequence identity to any one of (a)-(f).
405. The isolated AAV particle of embodiment 403 or 404, wherein the antibody molecule comprises a VH sequence encoded by the nucleotide sequence of SEQ ID NO: 1563 and a VL sequence encoded by the nucleotide sequence of SEQ ID NO: 1574.
406. The isolated AAV particle of any one of embodiments 1-391, wherein the antibody molecule comprises:
   (a) the HCDR1, HCDR2, and/or HCDR3 sequences of SEQ ID NOs: 1777, 1778, and 1779, respectively, and/or the LCDR1, LCDR2, and/or LCDR3 sequences of SEQ ID NOs: 1889, 1890, and 1891, respectively;
   (b) the HCDR1, HCDR2, and/or HCDR3 sequences of SEQ ID NOs: 1790, 1792, and 1794, respectively, and/or the LCDR1, LCDR2, and/or LCDR3 sequences of SEQ ID NOs: 1908, 1910, and 1912, respectively;
   (c) the HCDR1, HCDR2, and/or HCDR3 sequences of SEQ ID NOs: 1761, 1762, and 1763, respectively, and/or the LCDR1, LCDR2, and/or LCDR3 sequences of SEQ ID NOs: 1880, 1881, and 1882, respectively; or
   (d) the HCDR1, HCDR2, and/or HCDR3 sequences, and/or LCDR1, LCDR2, and/or LCDR3 sequences of any one of (a)-(c), with at most one, two, or three substitutions (e.g., conservative amino acid substitutions) in each CDR.
407. The isolated AAV particle of any one of embodiments 1-367, wherein the antibody molecule binds to all or a portion of amino acid residues of tau selected from: (a) 183-212, (b) 187-218, (c) 33-82, 159-182, 197-226, and 229-246; (d) 217-242, (e) 35-76 and 187-218, (f) 5-34, (g) 187-218, (h) 33-82, 159-188, and 191-230, (i) 35-62, 107-124, and 203-220, (j) 35-82, 159-188, and 197-224, and (k) 53-78, 329-348, or 381-408, wherein human tau is numbered according to SEQ ID NO: 9200.
408. The isolated AAV particle of embodiment 407, wherein one or more of the serines, threonines, and/or tyrosines in the stretch of amino acids selected from (a)-(k) are phosphorylated.
409. The isolated AAV particle of embodiment 407 or 408, wherein all of the serines, threonines, and/or tyrosines in the stretch of amino acids selected from (a)-(k) are phosphorylated.
410. The isolated AAV particle of any one of embodiments 1-376 or 407-409, wherein the antibody molecule binds to all or a portion of amino acids 195-215 of tau, e.g., with a dissociation constant (KD) of about 1 pM to about 50 pM, or about 1-25 pM, e.g., as assessed by bio-layer interferometry.
411. The isolated AAV particle of any one of embodiments 1-376 or 407-409, wherein the antibody molecule binds to all or a portion of amino acids 191-214 of tau phosphorylated at S199, e.g., with a dissociation constant (KD) of about 0.1 nM to about 10 nM, or about 0.5-5 nM, e.g., as assessed by bio-layer interferometry.
412. The isolated AAV particle of any one of embodiments 1-376 or 407-409, wherein the antibody molecule binds to all or a portion of amino acids 217-234 of tau phosphorylated at T217, T220, and T231, e.g., with a dissociation constant (KD) of about 0.1 nM to about 10 nM, or about 0.1-5 nM, e.g., as assessed by bio-layer interferometry.
413. The isolated AAV particle of any one of embodiments 1-376 or 407-409, wherein the antibody molecule binds to all or a portion of amino acids 225-240 of tau phosphorylated at T231, e.g., with a dissociation constant (KD) of about 0.1 nM to about 25 nM, or about 0.1-15 nM, e.g., as assessed by bio-layer interferometry.
414. The isolated AAV particle of any one of embodiments 1-376 or 407-409, wherein the antibody molecule binds to human tau phosphorylated at amino acid residue 5404, or a peptide comprising or consisting of the amino acid sequence DHGAEIVYKSPVVSGDT(pS)PRHLSNVSSTG (SEQ ID NO: 2143), wherein p(S) corresponds to a phosphorylated serine residue, optionally wherein the antibody molecule comprises a heavy chain variable region (VH) comprising HCDR1, HCDR2, and HCDR3 sequences comprising SEQ ID NOs: 2430, 2431, and 2432, respectively, and a light chain variable region (VL) comprising LCDR1, LCDR2, and LCDR3 sequences comprising SEQ ID NOs: 2435, 2436, and 2437, respectively.
415. The isolated AAV particle of any one of embodiments 1-376 or 407-409, wherein the antibody molecule binds to:
   (a) human tau phosphorylated at amino acid residue S199, but not at amino acid residues S202 and T205,
   (b) human tau phosphorylated at amino acid residue S202, but not at amino acid residues S199 and T205,
   (c) human tau phosphorylated at amino acid residue T205, but not at amino acid residues S199 and S202,
   (d) human tau phosphorylated at a combination of amino acid residues S199 and T205, but not at amino acid residue S202 (e.g., wherein binding tau phosphorylated at a combination of S199 and T205 is at least 3-times stronger (e.g., at least 4-time stronger) than background (e.g., non-specific) level of binding, e.g., binding by hIgG1 isotype control),
   (e) human tau phosphorylated at a combination of amino acid residues S202 and T205, but not at amino acid residue S199, but not human tau phosphorylated at a combination of residues S199 and S202, but not T205,
   (f) human tau phosphorylated at a combination of amino acid residues (i) S202 and T205, but not S119, and (ii) S199 and T205, but not 5202, e.g., at least 2 times (e.g., at least 3 times, at least 4 times, at least 5 times, 2-6 times, 2-5 times, 2-4 times, 2-3 times, 3-5 times or 4-5 times) more strongly than background (e.g., non-specific) level of binding, e.g., binding by hIgG1 isotype control).
   (g) human tau phosphorylated at a combination of amino acid residues (i) S199 and S202, but not T205, (ii) S202 and T205, but not S199, (iii) S199 and T205, but not S202, and (iv) S199, S202, and T205 (e.g., wherein binding to phosphorylated tau is at least 1.6-times stronger (e.g., at least 1.7 times, at least 1.8 times, at least 1.9 times, at least 2 times, at least 3 times, 1.6-3 times, 1.6-2 times stronger) than background (e.g., non-specific) level of binding, e.g., binding by hIgG1 isotype control),
   (h) a peptide comprising or consisting of the amino acid sequence

(SEQ ID NO: 2146)
SGDRSGYS(pS)PGSPGTPGSRSRTPS,

(i) a peptide comprising or consisting of the amino acid sequence

(SEQ ID NO: 2147)
SGDRSGYSSPG(pS)PGTPGSRSRTPS,

(j) a peptide comprising or consisting of the amino acid sequence

(SEQ ID NO: 2148)
SGDRSGYSSPGSPG(pT)PGSRSRTPS,

(k) a peptide comprising or consisting of the amino acid sequence SGDRSGYS(pS)PGSPG(pT)PGSRSRTPS (SEQ ID NO: 2152) (e.g., wherein binding to the peptide is at least 3 times stronger (e.g., at least 4 times stronger) than background (e.g., non-specific) level of binding, e.g., binding by hIgG1 isotype control),
(l) a peptide comprising or consisting of the amino acid sequence SGDRSGYSSPG(pS)PG(pT)PGSRSRTPS (SEQ ID NO: 2151), but not a peptide comprising or consisting of the amino acid sequence SGDRSGYS(pS)PG(pS)PGTPGSRSRTPS (SEQ ID NO: 2150),
(m) peptides comprising or consisting of the amino acid sequences SGDRSGYSSPG(pS)PG(pT)PGSRSRTPS (SEQ ID NO: 2151) and SGDRSGYS(pS)PGSPG(pT)PGSRSRTPS (SEQ ID NO: 2152), wherein binding to the latter peptide is at least 2 times (e.g., at least 3 times, at least 4 times, at least 5 times, 2-6 times, 2-5 times, 2-4 times, 2-3 times, 3-5 times or 4-5 times) more stronger than background (e.g., non-specific) level of binding, e.g., binding by hIgG1 isotype control), or
(n) peptides comprising or consisting of the amino acid sequences SGDRSGYS(pS)PG(pS)PGTPGSRSRTPS (SEQ ID NO: 2150), SGDRSGYSSPG(pS)PG(pT)PGSRSRTPS (SEQ ID NO: 2151), SGDRSGYS(pS)PGSPG(pT)PGSRSRTPS (SEQ ID NO: 2152), and SGDRSGYS(pS)PG(pS)PG(pT)PGSRSRTPS (SEQ ID NO: 2149) (e.g., wherein binding to the peptides is at least 1.6 times stronger (e.g., at least 1.7 times, at least 1.8 times, at least 1.9 times, at least 2 times, at least 3 times, 1.6-4 times, 1.6-3 times stronger) than background (e.g., non-specific) level of binding, e.g., binding by hIgG1 isotype control),
wherein p(S) and p(T) correspond to a phosphorylated serine and phosphorylated threonine, respectively,
optionally wherein binding is assessed, e.g., using ELISA, and optionally wherein human tau has the sequence set forth in SEQ ID NO: 9200, and
optionally wherein the antibody molecule comprises:
(i) a heavy chain variable region (VH) comprising HCDR1, HCDR2, and HCDR3 sequences comprising SEQ ID NOs: 2200, 2201, and 2202, respectively, and a light chain variable region (VL) comprising LCDR1, LCDR2, and LCDR3 sequences comprising SEQ ID NOs: 2207, 2208, and 2209, respectively;
(ii) a heavy chain variable region (VH) comprising HCDR1, HCDR2, and HCDR3 sequences comprising SEQ ID NOs: 2218, 2219, and 2220, respectively, and a light chain variable region (VL) comprising LCDR1, LCDR2, and LCDR3 sequences comprising SEQ ID NOs: 2207, 2208, and 2224, respectively;
(iii) a heavy chain variable region (VH) comprising HCDR1, HCDR2, and HCDR3 sequences comprising SEQ ID NOs: 2248, 2219, and 2249, respectively, and a light chain variable region (VL) comprising LCDR1, LCDR2, and LCDR3 sequences comprising SEQ ID NOs: 2207, 2208, and 2224, respectively;
(iv) a heavy chain variable region (VH) comprising HCDR1, HCDR2, and HCDR3 sequences comprising SEQ ID NOs: 2273, 2274, and 2275, respectively, and a light chain variable region (VL) comprising LCDR1, LCDR2, and LCDR3 sequences comprising SEQ ID NOs: 2207, 2208, and 2277, respectively; or
(v) a heavy chain variable region (VH) comprising HCDR1, HCDR2, and HCDR3 sequences comprising SEQ ID NOs: 2371, 2372, and 2373, respectively, and a light chain variable region (VL) comprising LCDR1, LCDR2, and LCDR3 sequences comprising SEQ ID NOs: 2207, 2208, and 2224, respectively.
416. The isolated AAV particle of any one of embodiments 1-376 or 407-409, wherein the antibody molecule binds to:
   (a) human tau phosphorylated at amino acid residue S199, but not at amino acid residues S202 and T205, optionally wherein the antibody comprises a heavy chain variable region (VH) comprising HCDR1, HCDR2, and HCDR3 sequences comprising SEQ ID NOs: 2200, 2201, and 2202, respectively, and a light chain variable region (VL) comprising LCDR1, LCDR2, and LCDR3 sequences comprising SEQ ID NOs: 2207, 2208, and 2209, respectively;
   (b) human tau phosphorylated at amino acid residue S202, but not at amino acid residues S199 and T205, optionally wherein the antibody comprises (i) a heavy chain variable region (VH) comprising HCDR1, HCDR2, and HCDR3 sequences comprising SEQ ID NOs: 2218, 2219, and 2220, respectively, and a light chain variable region (VL) comprising LCDR1, LCDR2, and LCDR3 sequences comprising SEQ ID NOs: 2207, 2208, and 2224, respectively; (ii) a heavy chain variable region (VH) comprising HCDR1, HCDR2, and HCDR3 sequences comprising SEQ ID NOs: 2248, 2219, and 2249, respectively, and a light chain variable region (VL) comprising LCDR1, LCDR2, and LCDR3 sequences comprising SEQ ID NOs: 2207, 2208, and 2224, respectively; or (iii) a heavy chain variable region (VH) comprising HCDR1, HCDR2, and HCDR3 sequences comprising SEQ ID NOs: 2371, 2372, and 2373, respectively, and a light chain variable region (VL) comprising LCDR1, LCDR2, and LCDR3 sequences comprising SEQ ID NOs: 2207, 2208, and 2224, respectively;
   (c) human tau phosphorylated at amino acid residue T205, but not at amino acid residues S199 and S202, optionally wherein the antibody comprises (i) a heavy chain variable region (VH) comprising HCDR1, HCDR2, and HCDR3 sequences comprising SEQ ID NOs: 2218, 2219, and 2220, respectively, and a light chain variable region (VL) comprising LCDR1, LCDR2, and LCDR3 sequences comprising SEQ ID NOs: 2207, 2208, and 2224, respectively; (ii) a heavy chain variable region (VH) comprising HCDR1, HCDR2, and HCDR3 sequences comprising SEQ ID NOs: 2248, 2219, and 2249, respectively, and a light chain variable region (VL) comprising LCDR1, LCDR2, and LCDR3 sequences comprising SEQ ID NOs: 2207, 2208, and 2224, respectively; or (iii) a heavy chain variable region (VH) comprising HCDR1, HCDR2, and HCDR3 sequences comprising SEQ ID NOs: 2371, 2372, and 2373, respectively, and a light chain variable region (VL) comprising LCDR1, LCDR2, and LCDR3 sequences comprising SEQ ID NOs: 2207, 2208, and 2224, respectively;
   (d) human tau phosphorylated at a combination of amino acid residues S199 and T205, but not at amino acid residue S202 (e.g., wherein binding tau phosphorylated at a combination of S199 and T205 is at least 3-times stronger (e.g., at least 4-time stronger) than background (e.g., non-specific) level of binding, e.g., binding by hIgG1 isotype control), optionally wherein the antibody comprises (i) a heavy chain variable region (VH) comprising HCDR1, HCDR2, and HCDR3 sequences comprising SEQ ID NOs: 2218, 2219, and 2220, respectively, and a light chain variable region (VL) comprising LCDR1, LCDR2, and LCDR3 sequences comprising SEQ ID NOs: 2207, 2208, and 2224, respectively; (ii) a heavy chain variable region (VH) comprising HCDR1, HCDR2, and HCDR3 sequences comprising SEQ ID NOs: 2248, 2219, and 2249, respectively, and a light chain variable region (VL) comprising LCDR1, LCDR2, and LCDR3 sequences comprising SEQ ID NOs: 2207, 2208, and 2224, respectively; (iii) a heavy chain variable region (VH) comprising HCDR1, HCDR2, and HCDR3 sequences comprising SEQ ID NOs: 2200, 2201, and 2202, respectively, and a light chain variable region (VL) comprising LCDR1, LCDR2, and LCDR3 sequences comprising SEQ ID NOs: 2207, 2208, and 2209, respectively; or (iv) a heavy chain variable region (VH) comprising HCDR1, HCDR2, and HCDR3 sequences comprising SEQ ID NOs: 2371, 2372, and 2373, respectively, and a light chain variable region (VL) comprising LCDR1, LCDR2, and LCDR3 sequences comprising SEQ ID NOs: 2207, 2208, and 2224, respectively;
   (e) human tau phosphorylated at a combination of amino acid residues S202 and T205, but not at amino acid residue S199, but not human tau phosphorylated at a combination of residues S199 and S202, but not T205, optionally wherein the antibody comprises a heavy chain variable region (VH) comprising HCDR1, HCDR2, and HCDR3 sequences comprising SEQ ID NOs: 2273, 2274, and 2275, respectively, and a light chain variable region (VL) comprising LCDR1, LCDR2, and LCDR3 sequences comprising SEQ ID NOs: 2207, 2208, and 2277, respectively;
   (f) human tau phosphorylated at a combination of amino acid residues (i) S202 and T205, but not S119, and (ii) S199 and T205, but not S202, e.g., at least 2 times (e.g., at least 3 times, at least 4 times, at least 5 times, 2-6 times, 2-5 times, 2-4 times, 2-3 times, 3-5 times or 4-5 times) more strongly than background (non-specific) level of binding, e.g., binding by hIgG1 isotype control), optionally wherein the antibody comprises (i) a heavy chain variable region (VH) comprising HCDR1, HCDR2, and HCDR3 sequences comprising SEQ ID NOs: 2218, 2219, and 2220, respectively, and a light chain variable region (VL) comprising LCDR1, LCDR2, and LCDR3 sequences comprising SEQ ID NOs: 2207, 2208, and 2224, respectively; (ii) a heavy chain variable region (VH) comprising HCDR1, HCDR2, and HCDR3 sequences comprising SEQ ID NOs: 2248, 2219, and 2249, respectively, and a light chain variable region (VL) comprising LCDR1, LCDR2, and LCDR3 sequences comprising SEQ ID NOs: 2207, 2208, and 2224, respectively; (iii) a heavy chain variable region (VH) comprising HCDR1, HCDR2, and HCDR3 sequences comprising SEQ ID NOs: 2200, 2201, and 2202, respectively, and a light chain variable region (VL) comprising LCDR1, LCDR2, and LCDR3 sequences comprising SEQ ID NOs: 2207, 2208, and 2209, respectively; or (iv) a heavy chain variable region (VH) comprising HCDR1, HCDR2, and HCDR3 sequences comprising SEQ ID NOs: 2371, 2372, and 2373, respectively, and a light chain variable region (VL) comprising LCDR1, LCDR2, and LCDR3 sequences comprising SEQ ID NOs: 2207, 2208, and 2224, respectively;
   (g) human tau phosphorylated at a combination of amino acid residues (i) S199 and S202, but not T205, (ii) S202 and T205, but not S199, (iii) S199 and T205, but not S202, and (iv) S199, S202, and T205 (e.g., wherein binding to phosphorylated tau is at least 1.6-times stronger (e.g., at least 1.7 times, at least 1.8 times, at least 1.9 times, at least 2 times, at least 3 times, 1.6-3 times, 1.6-2 times stronger) than background (non-specific) level of binding, e.g., binding by hIgG1 isotype control), optionally wherein the antibody comprises (i) a heavy chain variable region (VH) comprising HCDR1, HCDR2, and HCDR3 sequences comprising SEQ ID NOs: 2218, 2219, and 2220, respectively, and a light chain variable region (VL) comprising LCDR1, LCDR2, and LCDR3 sequences comprising SEQ ID NOs: 2207, 2208, and 2224, respectively; (ii) a heavy chain variable region (VH) comprising HCDR1, HCDR2, and HCDR3 sequences comprising SEQ ID NOs: 2248, 2219, and 2249, respectively, and a light chain variable region (VL) comprising LCDR1, LCDR2, and LCDR3 sequences comprising SEQ ID NOs: 2207, 2208, and 2224, respectively; (iii) a heavy chain variable region (VH) comprising HCDR1, HCDR2, and HCDR3 sequences comprising SEQ ID NOs: 2200, 2201, and 2202, respectively, and a light chain variable region (VL) comprising LCDR1, LCDR2, and LCDR3 sequences comprising SEQ ID NOs: 2207, 2208, and 2209, respectively; or (iv) a heavy chain variable region (VH) comprising HCDR1, HCDR2, and HCDR3 sequences comprising SEQ ID NOs: 2371, 2372, and 2373, respectively, and a light chain variable region (VL) comprising LCDR1, LCDR2, and LCDR3 sequences comprising SEQ ID NOs: 2207, 2208, and 2224, respectively;
   (h) a peptide comprising or consisting of the amino acid sequence SGDRSGYS(pS)PGSPGTPGSRSRTPS (SEQ ID NO: 2146), optionally wherein the antibody comprises a heavy chain variable region (VH) comprising HCDR1, HCDR2, and HCDR3 sequences comprising SEQ ID NOs: 2200, 2201, and 2202, respectively, and a light chain variable region (VL) comprising LCDR1, LCDR2, and LCDR3 sequences comprising SEQ ID NOs: 2207, 2208, and 2209, respectively
   (i) a peptide comprising or consisting of the amino acid sequence SGDRSGYSSPG(pS)PGTPGSRSRTPS (SEQ ID NO: 2147), optionally wherein the antibody comprises (i) a heavy chain variable region (VH) comprising HCDR1, HCDR2, and HCDR3 sequences comprising SEQ ID NOs: 2218, 2219, and 2220, respectively, and a light chain variable region (VL) comprising LCDR1, LCDR2, and LCDR3 sequences comprising SEQ ID NOs: 2207, 2208, and 2224, respectively; (ii) a heavy chain variable region (VH) comprising HCDR1, HCDR2, and HCDR3 sequences comprising SEQ ID NOs: 2248, 2219, and 2249, respectively, and a light chain variable region (VL) comprising LCDR1, LCDR2, and LCDR3 sequences comprising SEQ ID NOs: 2207, 2208, and 2224, respectively; or (iii) a heavy chain variable region (VH) comprising HCDR1, HCDR2, and HCDR3 sequences comprising SEQ ID NOs: 2371, 2372, and 2373, respectively, and a light chain variable region (VL) comprising LCDR1, LCDR2, and LCDR3 sequences comprising SEQ ID NOs: 2207, 2208, and 2224, respectively;
   (j) a peptide comprising or consisting of the amino acid sequence SGDRSGYSSPGSPG(pT)PGSRSRTPS (SEQ ID NO: 2148), optionally wherein the antibody comprises (i) a heavy chain variable region (VH) comprising HCDR1, HCDR2, and HCDR3 sequences comprising SEQ ID NOs: 2218, 2219, and 2220, respectively, and a light chain variable region (VL) comprising LCDR1, LCDR2, and LCDR3 sequences comprising SEQ ID NOs: 2207, 2208, and 2224, respectively; (ii) a heavy chain variable region (VH) comprising HCDR1, HCDR2, and HCDR3 sequences comprising SEQ ID NOs: 2248, 2219, and 2249, respectively, and a light chain variable region (VL) comprising LCDR1, LCDR2, and LCDR3 sequences comprising SEQ ID NOs: 2207, 2208, and 2224, respectively; or (iii) a heavy chain variable region (VH) comprising HCDR1, HCDR2, and HCDR3 sequences comprising SEQ ID NOs: 2371, 2372, and 2373, respectively, and a light chain variable region (VL) comprising LCDR1, LCDR2, and LCDR3 sequences comprising SEQ ID NOs: 2207, 2208, and 2224, respectively;
   (k) a peptide comprising or consisting of the amino acid sequence SGDRSGYS(pS)PGSPG(pT)PGSRSRTPS (SEQ ID NO: 2152) (e.g., wherein binding to the peptide is at least 3 times stronger (e.g., at least 4 times stronger) than background (non-specific) level of binding, e.g., binding by hIgG1 isotype control), optionally wherein the antibody comprises (i) a heavy chain variable region (VH) comprising HCDR1, HCDR2, and HCDR3 sequences comprising SEQ ID NOs: 2218, 2219, and 2220, respectively, and a light chain variable region (VL) comprising LCDR1, LCDR2, and LCDR3 sequences comprising SEQ ID NOs: 2207, 2208, and 2224, respectively; (ii) a heavy chain variable region (VH) comprising HCDR1, HCDR2, and HCDR3 sequences comprising SEQ ID NOs: 2248, 2219, and 2249, respectively, and a light chain variable region (VL) comprising LCDR1, LCDR2, and LCDR3 sequences comprising SEQ ID NOs: 2207, 2208, and 2224, respectively; (iii) a heavy chain variable region (VH) comprising HCDR1, HCDR2, and HCDR3 sequences comprising SEQ ID NOs: 2200, 2201, and 2202, respectively, and a light chain variable region (VL) comprising LCDR1, LCDR2, and LCDR3 sequences comprising SEQ ID NOs: 2207, 2208, and 2209, respectively; or (iv) a heavy chain variable region (VH) comprising HCDR1, HCDR2, and HCDR3 sequences comprising SEQ ID NOs: 2371, 2372, and 2373, respectively, and a light chain variable region (VL) comprising LCDR1, LCDR2, and LCDR3 sequences comprising SEQ ID NOs: 2207, 2208, and 2224, respectively;
   (l) a peptide comprising or consisting of the amino acid sequence SGDRSGYSSPG(pS)PG(pT)PGSRSRTPS (SEQ ID NO: 2151), but not a peptide comprising or consisting of the amino acid sequence SGDRSGYS(pS)PG(pS)PGTPGSRSRTPS (SEQ ID NO: 2150), optionally wherein the antibody comprises a heavy chain variable region (VH) comprising HCDR1, HCDR2, and HCDR3 sequences comprising SEQ ID NOs: 2273, 2274, and 2275, respectively, and a light chain variable region (VL) comprising LCDR1, LCDR2, and LCDR3 sequences comprising SEQ ID NOs: 2207, 2208, and 2277, respectively
   (m) peptides comprising or consisting of the amino acid sequences SGDRSGYSSPG(pS)PG(pT)PGSRSRTPS (SEQ ID NO: 2151) and SGDRSGYS(pS)PGSPG(pT)PGSRSRTPS (SEQ ID NO: 2152), e.g., wherein binding to the latter peptide is at least 2 times (e.g., at least 3 times, at least 4 times, at least 5 times, 2-6 times, 2-5 times, 2-4 times, 2-3 times, 3-5 times or 4-5 times) more stronger than background (non-specific) level of binding, e.g., binding by hIgG1 isotype control), optionally wherein the antibody comprises (i) a heavy chain variable region (VH) comprising HCDR1, HCDR2, and HCDR3 sequences comprising SEQ ID NOs: 2218, 2219, and 2220, respectively, and a light chain variable region (VL) comprising LCDR1, LCDR2, and LCDR3 sequences comprising SEQ ID NOs: 2207, 2208, and 2224, respectively; (ii) a heavy chain variable region (VH) comprising HCDR1, HCDR2, and HCDR3 sequences comprising SEQ ID NOs: 2248, 2219, and 2249, respectively, and a light chain variable region (VL) comprising LCDR1, LCDR2, and LCDR3 sequences comprising SEQ ID NOs: 2207, 2208, and 2224, respectively; (iii) a heavy chain variable region (VH) comprising HCDR1, HCDR2, and HCDR3 sequences comprising SEQ ID NOs: 2200, 2201, and 2202, respectively, and a light chain variable region (VL) comprising LCDR1, LCDR2, and LCDR3 sequences comprising SEQ ID NOs: 2207, 2208, and 2209, respectively; or (iv) a heavy chain variable region (VH) comprising HCDR1, HCDR2, and HCDR3 sequences comprising SEQ ID NOs: 2371, 2372, and 2373, respectively, and a light chain variable region (VL) comprising LCDR1, LCDR2, and LCDR3 sequences comprising SEQ ID NOs: 2207, 2208, and 2224, respectively; or
   (n) peptides comprising or consisting of the amino acid sequences SGDRSGYS(pS)PG(pS)PGTPGSRSRTPS (SEQ ID NO: 2150), SGDRSGYSSPG(pS)PG(pT)PGSRSRTPS (SEQ ID NO: 2151), SGDRSGYS(pS)PGSPG(pT)PGSRSRTPS (SEQ ID NO: 2152), and SGDRSGYS(pS)PG(pS)PG(pT)PGSRSRTPS (SEQ ID NO: 2149) (e.g., wherein binding to the peptides is at least 1.6 times stronger (e.g., at least 1.7 times, at least 1.8 times, at least 1.9 times, at least 2 times, at least 3 times, 1.6-4 times, 1.6-3 times stronger) than background (non-specific) level of binding, e.g., binding by hIgG1 isotype control), optionally wherein the antibody comprises (i) a heavy chain variable region (VH) comprising HCDR1, HCDR2, and HCDR3 sequences comprising SEQ ID NOs: 2218, 2219, and 2220, respectively, and a light chain variable region (VL) comprising LCDR1, LCDR2, and LCDR3 sequences comprising SEQ ID NOs: 2207, 2208, and 2224, respectively; (ii) a heavy chain variable region (VH) comprising HCDR1, HCDR2, and HCDR3 sequences comprising SEQ ID NOs: 2248, 2219, and 2249, respectively, and a light chain variable region (VL) comprising LCDR1, LCDR2, and LCDR3 sequences comprising SEQ ID NOs: 2207, 2208, and 2224, respectively; (iii) a heavy chain variable region (VH) comprising HCDR1, HCDR2, and HCDR3 sequences comprising SEQ ID NOs: 2200, 2201, and 2202, respectively, and a light chain variable region (VL) comprising LCDR1, LCDR2, and LCDR3 sequences comprising SEQ ID NOs: 2207, 2208, and 2209, respectively; or (iv) a heavy chain variable region (VH) comprising HCDR1, HCDR2, and HCDR3 sequences comprising SEQ ID NOs: 2371, 2372, and 2373, respectively, and a light chain variable region (VL) comprising LCDR1, LCDR2, and LCDR3 sequences comprising SEQ ID NOs: 2207, 2208, and 2224, respectively;
   wherein p(S) and p(T) correspond to a phosphorylated serine and phosphorylated threonine, respectively,
   optionally wherein binding is assessed, e.g., using ELISA, and optionally wherein human tau has the sequence set forth in SEQ ID NO: 9200.
417. The isolated AAV particle of any one of embodiments 1-376 or 407-409, wherein the antibody molecule binds to:
   (a) tau phosphorylated at T217, but not at T212 or T214, or
   (b) peptides comprising or consisting of the sequences GTPGSRSRTPSLP(pT)PPTRE (SEQ ID NO: 2155) and GTPGSRSRTP(pS)LP(pT)PPTRE (SEQ ID NO: 2158), but not peptides comprising or consisting of the sequences GTPGSRSR(pT)PSLPTPPTRE (SEQ ID NO: 2153), GTPGSRSRTP(pS)LPTPPTRE (SEQ ID NO: 2154), and GTPGSRSR(pT)P(pS)LPTPPTRE (SEQ ID NO: 2156),
   wherein p(S) and p(T) correspond to a phosphorylated serine and phosphorylated threonine, respectively,
   optionally wherein binding of the antibody to tau or the peptide is at least 1.5 times stronger (e.g., at least 1.6 times, at least 1.7 times, at least 1.8 times, at least 1.9 times, at least 2 times, at least 3 times, at least 4 times, at least 5 times, at least 6 times, 1.5-4 times, 1.5-3, 4-6 times stronger) than background (non-specific) level of binding, e.g., binding by hIgG1 isotype control),
   optionally wherein binding of the antibody to tau or the peptide is assessed, e.g., using one point ELISA as described, e.g., in Example 8, and optionally wherein human tau has the sequence set forth in SEQ ID NO: 9200, and
   optionally wherein the antibody molecule comprises:
   (i) a heavy chain variable region (VH) comprising HCDR1, HCDR2, and HCDR3 sequences comprising SEQ ID NOs: 2232, 2233, and 2234, respectively, and a light chain variable region (VL) comprising LCDR1, LCDR2, and LCDR3 sequences comprising SEQ ID NOs: 2238, 2239, and 2240, respectively;
   (ii) a heavy chain variable region (VH) comprising HCDR1, HCDR2, and HCDR3 sequences comprising SEQ ID NOs: 2284, 2392, and 2393, respectively, and a light chain variable region (VL) comprising LCDR1, LCDR2, and LCDR3 sequences comprising SEQ ID NOs: 2404, 2405, and 2406, respectively; or
   (iii) a heavy chain variable region (VH) comprising HCDR1, HCDR2, and HCDR3 sequences comprising SEQ ID NOs: 2445, 2446, and 2447, respectively, and a light chain variable region (VL) comprising LCDR1, LCDR2, and LCDR3 sequences comprising SEQ ID NOs: 2452, 2421, and 2453, respectively.
418. The isolated AAV particle of any one of embodiments 1-376 or 407-417, wherein the antibody molecule comprises a heavy chain variable region (VH) comprising one, two, or three of a heavy chain complementary determining region 1 (HCDR1), a heavy chain complementary determining region 2 (HCDR2), and a heavy chain complementary determining region 3 (HCDR3), and/or a light chain variable region (VL) comprising one, two, or three of a light chain complementary determining region 1 (LCDR1), a light chain complementary determining region 2 (LCDR2), and a light chain complementary determining region 3 (LCDR3), e.g., CDR sequences according to the Chothia numbering system, wherein:
   (i) the HCDR1, HCDR2, HCDR3, LCDR1, LCDR2, and LCDR3 comprise the amino acid sequences of SEQ ID NOs: 2200, 2201, 2202, 2207, 2208, and 2209, respectively;
   (ii) the HCDR1, HCDR2, HCDR3, LCDR1, LCDR2, and LCDR3 comprise the amino acid sequences of SEQ ID NOs: 2232, 2233, 2234, 2238, 2239, and 2240, respectively;
   (iii) the HCDR1, HCDR2, HCDR3, LCDR1, LCDR2, and LCDR3 comprise the amino acid sequences of SEQ ID NOs: 2248, 2219, 2249, 2207, 2208, and 2224, respectively;
   (iv) the HCDR1, HCDR2, HCDR3, LCDR1, LCDR2, and LCDR3 comprise the amino acid sequences of SEQ ID NOs: 2218, 2219, 2220, 2207, 2208, and 2224, respectively;
   (v) the HCDR1, HCDR2, HCDR3, LCDR1, LCDR2, and LCDR3 comprise the amino acid sequences of SEQ ID NOs: 2200, 2257, 2258, 2262, 2263, and 2264, respectively;
   (vi) the HCDR1, HCDR2, HCDR3, LCDR1, LCDR2, and LCDR3 comprise the amino acid sequences of SEQ ID NOs: 2415, 2416, 2417, 2420, 2421, and 2422, respectively;
   (vii) the HCDR1, HCDR2, HCDR3, LCDR1, LCDR2, and LCDR3 comprise the amino acid sequences of SEQ ID NOs: 2273, 2274, 2275, 2207, 2208, and 2277, respectively;
   (viii) the HCDR1, HCDR2, HCDR3, LCDR1, LCDR2, and LCDR3 comprise the amino acid sequences of SEQ ID NOs: 2284, 2285, 2286, 2289, 2290, and 2291, respectively;
   (ix) the HCDR1, HCDR2, HCDR3, LCDR1, LCDR2, and LCDR3 comprise the amino acid sequences of SEQ ID NOs: 2284, 2299, 2300, 2302, 2303, and 2304, respectively;
   (x) the HCDR1, HCDR2, HCDR3, LCDR1, LCDR2, and LCDR3 comprise the amino acid sequences of SEQ ID NOs: 2313, 2314, 2315, 2319, 2320, and 2321, respectively;
   (xi) the HCDR1, HCDR2, HCDR3, LCDR1, LCDR2, and LCDR3 comprise the amino acid sequences of SEQ ID NOs: 2330, 2331, 2332, 2262, 2336, and 2337, respectively;
   (xii) the HCDR1, HCDR2, HCDR3, LCDR1, LCDR2, and LCDR3 comprise the amino acid sequences of SEQ ID NOs: 2345, 2346, 2347, 2351, 2352, and 2353, respectively;
   (xiii) the HCDR1, HCDR2, HCDR3, LCDR1, LCDR2, and LCDR3 comprise the amino acid sequences of SEQ ID NOs: 2345, 2346, 2347, 2361, 2362, and 2363, respectively;
   (xiv) the HCDR1, HCDR2, HCDR3, LCDR1, LCDR2, and LCDR3 comprise the amino acid sequences of SEQ ID NOs: 2371, 2372, 2373, 2207, 2208, and 2224, respectively;
   (xv) the HCDR1, HCDR2, HCDR3, LCDR1, LCDR2, and LCDR3 comprise the amino acid sequences of SEQ ID NOs: 2381, 2382, 2383, 2262, 2263, and 2385, respectively;
   (xvi) the HCDR1, HCDR2, HCDR3, LCDR1, LCDR2, and LCDR3 comprise the amino acid sequences of SEQ ID NOs: 2284, 2392, 2393, 2396, 2239, and 2397, respectively;
   (xvii) the HCDR1, HCDR2, HCDR3, LCDR1, LCDR2, and LCDR3 comprise the amino acid sequences of SEQ ID NOs: 2284, 2392, 2393, 2404, 2405, and 2406, respectively;
   (xviii) the HCDR1, HCDR2, HCDR3, LCDR1, LCDR2, and LCDR3 comprise the amino acid sequences of SEQ ID NOs: 2430, 2431, 2432, 2435, 2436, and 2437, respectively;
   (xix) the HCDR1, HCDR2, HCDR3, LCDR1, LCDR2, and LCDR3 comprise the amino acid sequences of SEQ ID NOs: 2445, 2446, 2447, 2452, 2421, and 2453, respectively;
   (xx) the HCDR1, HCDR2, HCDR3, LCDR1, LCDR2, and LCDR3 comprise the amino acid sequences of SEQ ID NOs: 2459, 2460, 2461, 2465, 2466, and 2467, respectively;
   (xxi) the HCDR1, HCDR2, HCDR3, LCDR1, LCDR2, and LCDR3 comprise the amino acid sequences of any of the HCDR and LCDR sequences provided in Tables 14-16; or
   (xxii) a variant, e.g., functional variant, of the antibodies of any one of (i)-(xxi), wherein any one, two, three, four, five or all of the HCDR1, HCDR2, HCDR3, LCDR1, LCDR2, and/or LCDR3 comprises one, two, or at most three substitutions (e.g., conservative substitutions); or wherein any one, two, three, four, five or all of the HCDR1, HCDR2, HCDR3, LCDR1, LCDR2, and/or LCDR3 comprises one, two, or at most three different amino acids relative to any of the sequences in (i)-(xxi).
419. The isolated AAV particle of embodiment 418, wherein the antibody molecule comprises the HCDR1, HCDR2, HCDR3, LCDR1, LCDR2, and LCDR3 sequences of any one of (i)-(xxii).
420. The isolated AAV particle of embodiment 1-376 or 407-419, wherein the antibody molecule comprises:
   (a) the HCDR1, HCDR2, HCDR3, LCDR1, LCDR2, and LCDR3 comprise the amino acid sequences of SEQ ID NOs: 2248, 2219, 2249, 2207, 2208, and 2224, respectively;
   (b) the HCDR1, HCDR2, HCDR3, LCDR1, LCDR2, and LCDR3 comprise the amino acid sequences of SEQ ID NOs: 2200, 2201, 2202, 2207, 2208, and 2209, respectively;
   (c) the HCDR1, HCDR2, HCDR3, LCDR1, LCDR2, and LCDR3 comprise the amino acid sequences of SEQ ID NOs: 2232, 2233, 2234, 2238, 2239, and 2240, respectively;
   (d) the HCDR1, HCDR2, HCDR3, LCDR1, LCDR2, and LCDR3 comprise the amino acid sequences of SEQ ID NOs: 2218, 2219, 2220, 2207, 2208, and 2224, respectively;
   (e) the HCDR1, HCDR2, HCDR3, LCDR1, LCDR2, and LCDR3 comprise the amino acid sequences of SEQ ID NOs: 2415, 2416, 2417, 2420, 2421, and 2422, respectively; or
   (f) the HCDR1, HCDR2, HCDR3, LCDR1, LCDR2, and LCDR3 comprise the amino acid sequences of SEQ ID NOs: 2200, 2257, 2258, 2262, 2263, and 2264, respectively.
421. The isolated AAV particle of any one of embodiments 1-376 or 407-420, wherein the antibody molecule comprises a heavy chain CDR1, CDR2, and CDR3, and/or a light chain CDR1, CDR2, and CDR3 of an antibody molecule comprising a heavy chain variable region (VH) and light chain variable region (VL) comprising: (i) SEQ ID NOs: 2214 and 2215, respectively; (ii) SEQ ID NOs: 2244 and 2245, respectively; (iii) SEQ ID NOs: 2253 and 2254, respectively; (iv) SEQ ID NOs: 2228 and 2229, respectively; (v) SEQ ID NOs: 2269 and 2270, respectively; (vi) SEQ ID NOs: 2426 and 2427, respectively; (vii) SEQ ID NOs: 2280 and 2281, respectively; (viii) SEQ ID NOs: 2295 and 2296, respectively; (ix) SEQ ID NOs: 2309 and 2310, respectively; (x) SEQ ID NOs: 2326 and 2327, respectively; (xi) SEQ ID NOs: 2341 and 2342, respectively; (xii) SEQ ID NOs: 2357 and 2358, respectively; (xiii) SEQ ID NOs: 2367 and 2368, respectively; (xiv) SEQ ID NOs: 2377 and 2378, respectively; (xv) SEQ ID NOs: 2388 and 2389, respectively; (xvi) SEQ ID NOs: 2400 and 2401, respectively; (xvii) SEQ ID NOs: 2411 and 2412, respectively; (xviii) SEQ ID NOs: 2441 and 2442, respectively; (xix) SEQ ID NOs: 2455 and 2456, respectively; or (xx) SEQ ID NOs: 2470 and 2471, respectively.
422. The isolated AAV particle of embodiment 421, wherein the CDR sequences are based on the Kabat numbering system, Chothia numbering system, or IMGT numbering system.
423. The isolated AAV particle of any one of embodiments 1-376 or 407-422, wherein the antibody molecule comprises a VH comprising:
   (i) the amino acid sequence of any VH provided in Table 14 or 15, or an amino acid sequence having at least 70%, 75%, 80%, 85%, 90%, 92%, 95%, 96%, 97%, 98%, or 99% sequence identity thereto;
   (ii) an amino acid sequence comprising at least one, two or three modifications, but not more than 30, 20 or 10 modifications relative to the amino acid sequence of any VH provided in Table 14 or 15;
   (iii) an amino acid sequence comprising at least one, two or three, but not more than 30, 20 or 10 different amino acids relative to any one of the amino acid sequences of any VH sequences provided in Table 14 or 15; or
   (iv) an amino acid sequence encoded by a nucleotide sequence of any VH provided in Table 14 or 15, or a nucleotide sequence having at least 70%, 75%, 80%, 85%, 90%, 92%, 95%, 96%, 97%, 98%, or 99% sequence identity thereto.
424. The isolated AAV particle of any one of embodiments 1-376 or 407-423, wherein the antibody molecule comprises a VH comprising:
   (i) the amino acid sequence of any of SEQ ID NOs: 2253, 2214, 2244, 2228, 2426, and 2269, or an amino acid sequence having at least 70%, 75%, 80%, 85%, 90%, 92%, 95%, 96%, 97%, 98%, or 99% sequence identity thereto;
   (ii) an amino acid sequence comprising at least one, two or three modifications, but not more than 30, 20 or 10 modifications of the amino acid sequence of any of SEQ ID NOs: 2253, 2214, 2244, 2228, 2426, and 2269;
   (iii) an amino acid sequence comprising at least one, two or three, but not more than 30, 20 or 10 different amino acids relative to any one of the amino acid sequences of any of SEQ ID NOs: 2253, 2214, 2244, 2228, 2426, and 2269; or
   (iv) an amino acid sequence encoded by a nucleotide sequence of any of SEQ ID NOs: 2255, 2216, 2246, 2230, 2428, and 2271, or a nucleotide sequence having at least 70%, 75%, 80%, 85%, 90%, 92%, 95%, 96%, 97%, 98%, or 99% sequence identity thereto.
425. The isolated AAV particle of any one of embodiments 1-376 or 407-424, wherein the antibody molecule comprises a VL comprising:
   (i) the amino acid sequence of any VL provided in Table 14 or 15, or an amino acid sequence having at least 70%, 75%, 80%, 85%, 90%, 92%, 95%, 96%, 97%, 98%, or 99% sequence identity thereto;
   (ii) an amino acid sequence comprising at least one, two or three modifications, but not more than 30, 20 or 10 modifications relative to the amino acid sequence of any VL provided in Table 14 or 15;
   (iii) an amino acid sequence comprising at least one, two or three, but not more than 30, 20 or 10 different amino acids relative to any one of the amino acid sequences of any VL sequences provided in Table 14 or 15; or
   (iv) an amino acid sequence encoded by a nucleotide sequence of any VL provided in Table 14 or 15, or a nucleotide sequence having at least 70%, 75%, 80%, 85%, 90%, 92%, 95%, 96%, 97%, 98%, or 99% sequence identity thereto.
426. The isolated AAV particle of any one of embodiments 1-376 or 407-425, wherein the antibody molecule comprises a VL comprising:
   (i) the amino acid sequence of any of SEQ ID NOs: 2254, 2215, 2245, 2229, 2427, and 2270, or an amino acid sequence having at least 70%, 75%, 80%, 85%, 90%, 92%, 95%, 96%, 97%, 98%, or 99% sequence identity thereto;
   (ii) an amino acid sequence comprising at least one, two or three modifications, but not more than 30, 20 or 10 modifications of the amino acid sequence of any of SEQ ID NOs: 2254, 2215, 2245, 2229, 2427, and 2270;
   (iii) an amino acid sequence comprising at least one, two or three, but not more than 30, 20 or 10 different amino acids relative to any one of the amino acid sequences of any of SEQ ID NOs: 2254, 2215, 2245, 2229, 2427, and 2270; or
   (iv) an amino acid sequence encoded by a nucleotide sequence of any of SEQ ID NOs: 2256, 2217, 2247, 2231, 2429, and 2272, or a nucleotide sequence having at least 70%, 75%, 80%, 85%, 90%, 92%, 95%, 96%, 97%, 98%, or 99% sequence identity thereto.
427. The isolated AAV particle of any one of embodiments 1-376 or 407-426, wherein the antibody molecule comprises:
(i) a VH comprising:
   (a) the amino acid sequence of any VH provided in Table 14 or 15, or an amino acid sequence having at least 70%, 75%, 80%, 85%, 90%, 92%, 95%, 96%, 97%, 98%, or 99% sequence identity thereto;
   (b) an amino acid sequence comprising at least one, two or three modifications, but not more than 30, 20 or 10 modifications relative to the amino acid sequence of any VH provided in Table 14 or 15;
   (c) an amino acid sequence comprising at least one, two or three, but not more than 30, 20 or 10 different amino acids relative to any one of the amino acid sequences of any VH sequences provided in Table 14 or 15; or
   (d) an amino acid sequence encoded by a nucleotide sequence of any VH provided in Table 14 or 15, or a nucleotide sequence having at least 70%, 75%, 80%, 85%, 90%, 92%, 95%, 96%, 97%, 98%, or 99% sequence identity thereto; and
(ii) a VL comprising:
   (a) the amino acid sequence of any VL provided in Table 14 or 15, or an amino acid sequence having at least 70%, 75%, 80%, 85%, 90%, 92%, 95%, 96%, 97%, 98%, or 99% sequence identity thereto;
   (b) an amino acid sequence comprising at least one, two or three modifications, but not more than 30, 20 or 10 modifications relative to the amino acid sequence of any VL provided in Table 14 or 15;
   (c) an amino acid sequence comprising at least one, two or three, but not more than 30, 20 or 10 different amino acids relative to any one of the amino acid sequences of any VL sequences provided in Table 14 or 15; or
   (d) an amino acid sequence encoded by a nucleotide sequence of any VL provided in Table 14 or 15, or a nucleotide sequence having at least 70%, 75%, 80%, 85%, 90%, 92%, 95%, 96%, 97%, 98%, or 99% sequence identity thereto.
428. The isolated AAV particle of any one of embodiments 1-376 or 407-427, wherein the antibody molecule comprises the amino acid sequence of any VH of an antibody provided in Table 14 or 15, and the amino acid sequence of the VL of the antibody provided in Table 14 or 15.
429. The isolated AAV particle of any one of embodiments 1-376 or 407-428, wherein the antibody molecule comprises:
   (i) a VH comprising the amino acid sequence of SEQ ID NO: 2253; an amino acid sequence having at least 70%, 75%, 80%, 85%, 90%, 92%, 95%, 96%, 97%, 98%, or 99% sequence identity thereto; an amino acid sequence comprising at least one, two or three, but not more than 30, 20 or 10 different amino acids relative to SEQ ID NO: 2253; or an amino acid sequence comprising at least one, two or three, but not more than 30, 20 or 10 modifications, e.g., substitutions (e.g., conservative substitutions) relative to SEQ ID NO: 2253; and
   (ii) a VL comprising the amino acid sequence of SEQ ID NO: 2254, or an amino acid sequence having at least 70%, 75%, 80%, 85%, 90%, 92%, 95%, 96%, 97%, 98%, or 99% sequence identity thereto; an amino acid sequence comprising at least one, two or three, but not more than 30, 20 or 10 different amino acids relative to SEQ ID NO: 2254; or an amino acid sequence comprising at least one, two or three, but not more than 30, 20 or 10 modifications, e.g., substitutions (e.g., conservative substitutions) relative to SEQ ID NO: 2254.
430. The isolated AAV particle of any one of embodiments 1-376 or 407-428, wherein the antibody molecule comprises:
   (i) a VH comprising the amino acid sequence of SEQ ID NO: 2214; an amino acid sequence having at least 70%, 75%, 80%, 85%, 90%, 92%, 95%, 96%, 97%, 98%, or 99% sequence identity thereto; an amino acid sequence comprising at least one, two or three, but not more than 30, 20 or 10 different amino acids relative to SEQ ID NO: 2214; or an amino acid sequence comprising at least one, two or three, but not more than 30, 20 or 10 modifications, e.g., substitutions (e.g., conservative substitutions) relative to SEQ ID NO: 2214; and
   (ii) a VL comprising the amino acid sequence of SEQ ID NO: 2215, or an amino acid sequence having at least 70%, 75%, 80%, 85%, 90%, 92%, 95%, 96%, 97%, 98%, or 99% sequence identity thereto; an amino acid sequence comprising at least one, two or three, but not more than 30, 20 or 10 different amino acids relative to SEQ ID NO: 2215; or an amino acid sequence comprising at least one, two or three, but not more than 30, 20 or 10 modifications, e.g., substitutions (e.g., conservative substitutions) relative to SEQ ID NO: 2215.
431. The isolated AAV particle of any one of embodiments 1-376 or 407-428, wherein the antibody molecule comprises:
   (i) a VH comprising the amino acid sequence of SEQ ID NO: 2244; an amino acid sequence having at least 70%, 75%, 80%, 85%, 90%, 92%, 95%, 96%, 97%, 98%, or 99% sequence identity thereto; an amino acid sequence comprising at least one, two or three, but not more than 30, 20 or 10 different amino acids relative to SEQ ID NO: 2244; or an amino acid sequence comprising at least one, two or three, but not more than 30, 20 or 10 modifications, e.g., substitutions (e.g., conservative substitutions) relative to SEQ ID NO: 2244; and
   (ii) a VL comprising the amino acid sequence of SEQ ID NO: 2245, or an amino acid sequence having at least 70%, 75%, 80%, 85%, 90%, 92%, 95%, 96%, 97%, 98%, or 99% sequence identity thereto; an amino acid sequence comprising at least one, two or three, but not more than 30, 20 or 10 different amino acids relative to SEQ ID NO: 2245; or an amino acid sequence comprising at least one, two or three, but not more than 30, 20 or 10 modifications, e.g., substitutions (e.g., conservative substitutions) relative to SEQ ID NO: 2245.
432. The isolated AAV particle of any one of embodiments 1-376 or 407-428, wherein the antibody molecule comprises:
   (i) a VH comprising the amino acid sequence of SEQ ID NO: 2228; an amino acid sequence having at least 70%, 75%, 80%, 85%, 90%, 92%, 95%, 96%, 97%, 98%, or 99% sequence identity thereto; an amino acid sequence comprising at least one, two or three, but not more than 30, 20 or 10 different amino acids relative to SEQ ID NO: 2228; or an amino acid sequence comprising at least one, two or three, but not more than 30, 20 or 10 modifications, e.g., substitutions (e.g., conservative substitutions) relative to SEQ ID NO: 2228; and
   (ii) a VL comprising the amino acid sequence of SEQ ID NO: 2229, or an amino acid sequence having at least 70%, 75%, 80%, 85%, 90%, 92%, 95%, 96%, 97%, 98%, or 99% sequence identity thereto; an amino acid sequence comprising at least one, two or three, but not more than 30, 20 or 10 different amino acids relative to SEQ ID NO: 2229; or an amino acid sequence comprising at least one, two or three, but not more than 30, 20 or 10 modifications, e.g., substitutions (e.g., conservative substitutions) relative to SEQ ID NO: 2229.
433. The isolated AAV particle of any one of embodiments 1-376 or 407-428, wherein the antibody molecule comprises:
   (i) a VH comprising the amino acid sequence of SEQ ID NO: 2269; an amino acid sequence having at least 70%, 75%, 80%, 85%, 90%, 92%, 95%, 96%, 97%, 98%, or 99% sequence identity thereto; an amino acid sequence comprising at least one, two or three, but not more than 30, 20 or 10 different amino acids relative to SEQ ID NO: 2269; or an amino acid sequence comprising at least one, two or three, but not more than 30, 20 or 10 modifications, e.g., substitutions (e.g., conservative substitutions) relative to SEQ ID NO: 2269; and
   (ii) a VL comprising the amino acid sequence of SEQ ID NO: 2270, or an amino acid sequence having at least 70%, 75%, 80%, 85%, 90%, 92%, 95%, 96%, 97%, 98%, or 99% sequence identity thereto; an amino acid sequence comprising at least one, two or three, but not more than 30, 20 or 10 different amino acids relative to SEQ ID NO: 2270; or an amino acid sequence comprising at least one, two or three, but not more than 30, 20 or 10 modifications, e.g., substitutions (e.g., conservative substitutions) relative to SEQ ID NO: 2270.
434. The isolated AAV particle of any one of embodiments 1-376 or 407-428, wherein the antibody molecule comprises:
   (i) a VH comprising the amino acid sequence of SEQ ID NO: 2426; an amino acid sequence having at least 70%, 75%, 80%, 85%, 90%, 92%, 95%, 96%, 97%, 98%, or 99% sequence identity thereto; an amino acid sequence comprising at least one, two or three, but not more than 30, 20 or 10 different amino acids relative to SEQ ID NO: 2426; or an amino acid sequence comprising at least one, two or three, but not more than 30, 20 or 10 modifications, e.g., substitutions (e.g., conservative substitutions) relative to SEQ ID NO: 2426; and
   (ii) a VL comprising the amino acid sequence of SEQ ID NO: 2427, or an amino acid sequence having at least 70%, 75%, 80%, 85%, 90%, 92%, 95%, 96%, 97%, 98%, or 99% sequence identity thereto; an amino acid sequence comprising at least one, two or three, but not more than 30, 20 or 10 different amino acids relative to SEQ ID NO: 2427; or an amino acid sequence comprising at least one, two or three, but not more than 30, 20 or 10 modifications, e.g., substitutions (e.g., conservative substitutions) relative to SEQ ID NO: 2427.
435. The isolated AAV particle of any one of embodiments 1-376 or 407-434, wherein the antibody molecule comprises a heavy chain variable region (VH) and/or a light chain variable region (VL) comprising: (i) SEQ ID NOs: 2214 and 2215, respectively; (ii) SEQ ID NOs: 2244 and 2245, respectively; (iii) SEQ ID NOs: 2253 and 2254, respectively; (iv) SEQ ID NOs: 2228 and 2229, respectively; (v) SEQ ID NOs: 2269 and 2270, respectively; (vi) SEQ ID NOs: 2426 and 2427, respectively; (vii) SEQ ID NOs: 2280 and 2281, respectively; (viii) SEQ ID NOs: 2295 and 2296, respectively; (ix) SEQ ID NOs: 2309 and 2310, respectively; (x) SEQ ID NOs: 2326 and 2327, respectively; (xi) SEQ ID NOs: 2341 and 2342, respectively; (xii) SEQ ID NOs: 2357 and 2358, respectively; (xiii) SEQ ID NOs: 2367 and 2368, respectively; (xiv) SEQ ID NOs: 2377 and 2378, respectively; (xv) SEQ ID NOs: 2388 and 2389, respectively; (xvi) SEQ ID NOs: 2400 and 2401, respectively; (xvii) SEQ ID NOs: 2411 and 2412, respectively; (xviii) SEQ ID NOs: 2441 and 2442, respectively; (xix) SEQ ID NOs: 2455 and 2456, respectively; (xx) SEQ ID NOs: 2470 and 2471, respectively, (xxi) a variant, e.g., functional variant, of the antibodies of any one of (i)-(xx), wherein the VH and/or VL has an amino acid sequence having at least 70%, 75%, 80%, 85%, 90%, 92%, 95%, 96%, 97%, 98%, or 99% sequence identity thereto; or (xxii) a variant, e.g., functional variant, of the antibodies of any one of (i)-(xx), wherein the VH and/or VL comprises at least one, two, or three modifications, but not more than 30, 20, or 10 modifications (e.g., amino acid substitutions, e.g., conservative substitutions) or wherein the VH and/or VL comprises at least one, two, or three, but not more than 30, 20, or 10 different amino acids.
436. The isolated AAV particle of embodiment 435, wherein the antibody molecule comprises the VH and VL sequences of any one of (i)-(xxii).
437. The isolated AAV particle of any one of embodiments 1-376 or 407-436, wherein the antibody molecule comprises VH and VL sequences comprising the amino acid sequences of:
   (a) SEQ ID NOs: 2253 and 2254, respectively,
   (b) SEQ ID NOs: 2214 and 2215, respectively,
   (c) SEQ ID NOs: 2244 and 2245, respectively,
   (d) SEQ ID NOs: 2228 and 2229, respectively,
   (e) SEQ ID NOs: 2426 and 2427, respectively, or
   (f) SEQ ID NOs: 2269 and 2270, respectively.
438. The isolated AAV particle of embodiment 1-376 or 407-437, wherein the antibody molecule comprises a VH sequence encoded by a nucleotide sequence having at least 80% (e.g., 85, 90, 95, 96, 97, 98, or 99%) sequence identity to any one of SEQ ID NOs: 2255, 2216, 2246, 2230, 2428, 2271, 2282, 2297, 2311, 2328, 2343, 2359, 2369, 2379, 2390, 2402, 2413, 2443, 2457, or 2472 and/or a VL sequence encoded by a nucleotide sequence having at least 80% (e.g., 85, 90, 95, 96, 97, 98, or 99%) sequence identity to any one of SEQ ID NOs: 2256, 2217, 2247, 2231, 2429, 2272, 2283, 2298, 2312, 2329, 2344, 2360, 2370, 2380, 2391, 2403, 2414, 2444, 2458, or 2473.
439. The isolated AAV particle of embodiment 438, wherein the antibody molecule comprises VH and VL sequences encoded by nucleotide sequences set forth in (a) SEQ ID NOs: 2255 and 2256, respectively, (b) SEQ ID NOs: 2216 and 2217, respectively, (c) SEQ ID NOs: 2246 and 2247, respectively, (d) SEQ ID NOs: 2230 and 2231, respectively, (e) SEQ ID NOs: 2428 and 2429, respectively, (f) SEQ ID NOs: 2271 and 2272, respectively, (g) SEQ ID NOs: 2282 and 2283, respectively, (h) SEQ ID NOs: 2297 and 2298, respectively, (i) SEQ ID NOs: 2311 and 2312, respectively, (j) SEQ ID NOs: 2328 and 2329, respectively, (k) SEQ ID NOs: 2343 and 2344, respectively, (1) SEQ ID NOs: 2359 and 2360, respectively, (m) SEQ ID NOs: 2369 and 2370, respectively, (n) SEQ ID NOs: 2379 and 2380, respectively, (o) SEQ ID NOs: 2390 and 2391, respectively, (p) SEQ ID NOs: 2402 and 2403, respectively, (q) SEQ ID NOs: 2413 and 2414, respectively, (r) SEQ ID NOs: 2443 and 2444, respectively, (s) SEQ ID NOs: 2457 and 2458, respectively, (t) SEQ ID NOs: 2472 and 2473, respectively, or (u) a nucleotide sequence having at least 80% (e.g., 85, 90, 95, 96, 97, 98, or 99%) sequence identity to any one of (a)-(t).
440. The isolated AAV particle of any one of embodiments 1-439, wherein the antibody molecule comprises:
   (i) a heavy chain constant region comprising an amino acid sequence of any of the heavy chain constant region sequences in Table 17, or a sequence having at least 80% (e.g., 85, 90, 95, 96, 97, 98, or 99%) sequence identity to the heavy chain constant region sequences in Table 17, or an amino acid sequence having at least one, two or three modifications, but not more than 30, 20 or 10 modifications, to the amino acid sequence of the heavy chain constant region sequences in Table 17; and/or
   (ii) a light chain constant region (CL) comprising an amino acid sequence of any of the CL sequences in Table 17, or a sequence having at least 80% (e.g., 85, 90, 95, 96, 97, 98, or 99%) sequence identity to any of the CL sequences in Table 17, or an amino acid sequence having at least one, two or three modifications, but not more than 30, 20 or 10 modifications, to the amino acid sequence of the light chain constant region sequences in Table 17.
441. The isolated AAV particle of any one of embodiments 1-440, wherein the antibody molecule comprises:
   (i) a heavy chain (HC) comprising the amino acid sequence of the HC of any one of the anti-tau antibody molecules described herein (e.g., the HC of any one of the anti-tau antibody molecules listed in Tables 7-16), or a sequence having at least 80% (e.g., 85, 90, 95, 96, 97, 98, or 99%) sequence identity to the HC sequence of any of the anti-tau antibody molecules described herein (e.g., the HC of any one of the anti-tau antibody molecules listed in Tables 7-16), or an amino acid sequence having at least one, two or three modifications, but not more than 30, 20 or 10 modifications, to the amino acid sequence of the HC sequence of any of the anti-tau antibody molecules described herein (e.g., the HC of any one of the anti-tau antibody molecules listed in Tables 7-16); and/or
   (ii) a light chain (LC) comprising the amino acid sequence of the LC of any of any one of the anti-tau antibody molecules described herein (e.g., the LC of any one of the anti-tau antibody molecules listed in Tables 7-16), or a sequence having at least 80% (e.g., 85, 90, 95, 96, 97, 98, or 99%) sequence identity to the LC sequence of any of the anti-tau antibody molecules described herein (e.g., the LC of any one of the anti-tau antibody molecules listed in Tables 7-16), or an amino acid sequence having at least one, two or three modifications, but not more than 30, 20 or 10 modifications, to the amino acid sequence of the LC sequence of any of the anti-tau antibody molecules described herein (e.g., the LC of any one of the anti-tau antibody molecules listed in Tables 7-16).
442. The isolated AAV particle of any one of embodiments 1-441, wherein the antibody molecule competes for binding to tau with an antibody as defined in any one of embodiments 167-198.
443. The isolated AAV particle of any one of embodiments 1-442, wherein the antibody molecule binds to the same epitope, substantially the same epitope as, an epitope that overlaps with, or an epitope that substantially overlaps with an antibody as defined in any one of embodiments 167-198.
444. The isolated AAV particle of any one of embodiments 1-443, wherein the antibody molecule is a full length antibody, a bispecific antibody, a Fab, a F(ab′)2, a Fv, a single chain Fv fragment (scFv), single domain antibody, or a camelid antibody.
445. The isolated AAV particle of any one of embodiments 1-444, wherein the antibody molecule comprises a heavy chain constant region selected from human IgG1, human IgG2, human IgG3, human IgG4, murine IgG1, murine IgG2a, murine IgG2b, murine IgG2c, and murine IgG3; and/or a light chain constant region selected from the light chain constant regions of kappa or lambda.
446. The isolated AAV particle of any one of embodiments 1-445, wherein the antibody molecule comprises an Fc region or functional variant thereof.
447. The isolated AAV particle of any one of embodiments 1-446, wherein the antibody molecule comprises an Fc region which has reduced affinity, e.g., ablated, affinity for an Fc receptor, e.g., as compared to a reference, wherein the reference is a wild-type Fc receptor.
448. The isolated AAV particle of any one of embodiments 1-447, wherein the antibody molecule comprises an Fc region which comprises a mutation at one, two, or all of positions I253 (e.g., I235A), H310 (e.g., H310A or H310Q), and/or H435 (e.g., H435A or H435Q), numbered according to the EU index as in Kabat.
449. The isolated AAV particle of any one of embodiments 1-448, wherein the antibody molecule comprises an Fc region which has reduced effector function (e.g., reduced ADCC), compared to a reference wherein the reference is a wild-type Fc receptor.
450. The isolated AAV particle of any one of embodiments 1-449, wherein the antibody molecule comprises an Fc region which comprises a mutation at one, two, three, four, or all of positions L235 (e.g., L235V), F243 (e.g., F243L), R292 (e.g., R292P), Y300 (e.g., Y300L), and P396 (e.g., P396L), numbered according to the EU index as in Kabat.
451. The isolated AAV particle of any one of embodiments 1-450, wherein the antibody molecule comprises a signal sequence.
452. The isolated AAV particle of embodiment 451, wherein, in the antibody molecule,
   (i) the signal sequence is located 5′ relative to the VH and/or the heavy chain; and/or
   (ii) the signal sequence is located 5′ relative to the VL and/or the light chain.
453. The isolated AAV particle of any one of embodiments 1-367, wherein the antibody molecule is selected from E2814, semorinemab (RG6100, R07105705, MTAU9937A), gosuranemab (BIIB092, BMS-986168, IPN007/IPN002), tilavonemab (ABBV-8E12, C2N-8E12, HJ8.5), zagotenemab (LY3303560, MC-1 IgG1), posdinemab (JNJ-63733657, B296, PT3), bepranemab (UCB0107), BIIB076 (N1-105.6C5 huIgG1λ), RG7345 (R06926496), UCB0107 (D IgG4), NPT088, PNT001, Lu AF87908, PRX005, or APNmAb005.
454. The isolated AAV particle of any one of embodiments 1-453, wherein, in the antibody molecule,
   (i) the VH and VL are connected directly, e.g., without a linker;
   (ii) the VH and VL are connected via a linker (e.g., a linker listed in Table 19);
   (iii) the heavy chain and light chain are connected directly, e.g., without a linker; or
   (iv) the heavy chain and light chain are connected via a linker (e.g., a linker listed in Table 19).
455. The isolated AAV particle of embodiment 454, wherein:
   (i) the linker comprises an encoded furin cleavage site;
   (ii) the linker comprises an encoded T2A linker; and/or
   (iii) the linker comprises a glycine-serine linker, e.g., a G4S linker or a (G4S)3 linker.
456. The isolated AAV particle of any one of embodiments 1-455, wherein the antibody molecule comprises a second antigen-binding region having a different binding specificity than the antigen-binding region that binds to tau.
457. The isolated AAV particle of any one of embodiments 1-456, wherein the antibody molecule is a multispecific antibody molecule comprising at least a first antigen-binding domain and a second antigen-binding domain, e.g., a bispecific antibody molecule.
458. The isolated AAV particle of embodiment 1-457, wherein the first and/or second antigen binding domain of the antibody molecule comprise an IgG antibody, single-chain Fv (scFv), a scFv fragment, a Fab, an F(ab′)₂, a single-chain Fab (scFabs), a single-chain antibody, a diabody, a bispecific antibody, an antibody variable domain, a VHH, a single domain antibody, a camelid antibody, an intrabody, and/or a nanobody.
459. The isolated AAV particle of any one of the preceding embodiments, which comprises a viral genome comprising a promoter operably linked to the nucleic acid sequence encoding the antibody molecule, wherein the promoter comprises a tissue specific promoter or a ubiquitous promoter.
460. The isolated AAV particle of embodiment 459, wherein the promoter is chosen from human elongation factor 1α-subunit (EF1α), cytomegalovirus (CMV) immediate-early enhancer and/or promoter, chicken β-actin (CBA) and its derivative CAG, β glucuronidase (GUSB), or ubiquitin C (UBC), neuron-specific enolase (NSE), platelet-derived growth factor (PDGF), platelet-derived growth factor B-chain (PDGF-β), intercellular adhesion molecule 2 (ICAM-2), synapsin (Syn), methyl-CpG binding protein 2 (MeCP2), Ca2+/calmodulin-dependent protein kinase II (CaMKII), metabotropic glutamate receptor 2 (mGluR2), neurofilament light (NFL) or heavy (NFH), β-globin minigene nβ2, preproenkephalin (PPE), enkephalin (Enk) and excitatory amino acid transporter 2 (EAAT2), glial fibrillary acidic protein (GFAP), myelin basic protein (MBP), a cardiovascular promoter (e.g., αMHC, cTnT, and CMV-MLC2k), a liver promoter (e.g., hAAT, TBG), a skeletal muscle promoter (e.g., desmin, MCK, C512) or a fragment, e.g., a truncation, or a functional variant thereof.
461. The isolated AAV particle of embodiment 460, wherein the promoter is an EF-1a promoter variant, e.g., a truncated EF-1a promoter.
462. The isolated AAV particle of any one of embodiments 459-461, wherein the promoter comprises the nucleotide sequence of any one of SEQ ID NOs: 9000, 9001, 9003, 9004, 9008, 9009, 9011-9020, GCATG, CGTGAG, GT, GCTCCGGT, or GTAAG, a nucleotide sequence comprising at least one, two, or three but no more than four modifications, e.g., substitutions (e.g., conservative substitutions), insertions, or deletions, relative to the nucleotide sequence of SEQ ID NOs: 9000, 9001, 9003, 9004, 9008, 9009, 9011-9020, GCATG, CGTGAG, GT, GCTCCGGT, or GTAAG, or a nucleotide sequence with at least 80% (e.g., 85%, 90%, 95%, 96%, 97%, 98%, or 99%) sequence identity to any one of SEQ ID NOs: 9000, 9001, 9003, 9004, 9008, 9009, 9011-9020, GCATG, CGTGAG, GT, GCTCCGGT, or GTAAG.
463. The isolated AAV particle of any one of embodiments 459-462, wherein the viral genome further comprises a polyA signal sequence.
464. The isolated AAV particle of any one of embodiments 459-463, wherein the viral genome further comprises an inverted terminal repeat (ITR) sequence.
465. The isolated AAV particle of any one of embodiments 459-464, wherein the viral genome comprises an ITR sequence positioned 5′ relative to the encoded antibody molecule.
466. The isolated AAV particle of any one of embodiments 459-465, wherein the viral genome comprises an ITR sequence positioned 3′ relative to the encoded antibody molecule.
467. The isolated AAV particle of any one of embodiments 459-466, wherein the viral genome comprises an ITR sequence positioned 5′ relative to the encoded antibody molecule and an ITR sequence positioned 3′ relative to the encoded antibody molecule.
468. The isolated AAV particle of any one of embodiments 459-467, wherein the viral genome further comprises an enhancer, a Kozak sequence (e.g., GCCGCCACCATG (SEQ ID NO: 9021) or GAGGAGCCACC (SEQ ID NO: 9022), an intron region, and/or an exon region.
469. The isolated AAV particle of any one of embodiments 459-468, wherein the viral genome comprises:
   (a) one or more inverted terminal repeat (ITR) sequences, optionally wherein the ITR sequence is positioned 5′ relative to the transgene encoding the antibody molecule and/or the ITR sequence is positioned 3′ relative to the transgene encoding the antibody molecule, optionally wherein the ITR comprises the nucleotide sequence of SEQ ID NO: 2026 and/or 2039,
   (b) an enhancer, optionally wherein the enhancer comprises the nucleotide sequence of SEQ ID NO: 2027,
   (d) an intron region, optionally wherein the intron region comprises the nucleotide sequence of SEQ ID NO: 2029,
   (e) a nucleotide sequence encoding one or more signal sequences, optionally wherein the signal sequence comprises the nucleotide sequence of SEQ ID NO: 2030 and/or 2035,
   (f) a nucleotide sequence encoding a heavy chain variable region (VH) of the antibody molecule, optionally wherein the VH is encoded by the nucleotide sequence of SEQ ID NO: 2031,
   (g) a nucleotide sequence encoding an IgG1 heavy chain constant region, optionally wherein the IgG1 heavy chain constant region is encoded by the nucleotide sequence of SEQ ID NO: 2032,
   (h) a furin cleavage site, optionally wherein the furin cleavage site comprises the nucleotide sequence of SEQ ID NO: 2033,
   (i) a linker, optionally wherein the linker is a T2A linker, optionally wherein the T2A linker comprises the nucleotide sequence of SEQ ID NO: 2034;
   (j) a nucleotide sequence encoding a light chain variable region (VL), optionally wherein the VL is encoded by the nucleotide sequence of SEQ ID NO: 2036;
   (k) a nucleotide sequence encoding a IgG kappa light chain constant region, optionally wherein the IgG kappa light chain constant region is encoded by the nucleotide sequence of SEQ ID NO: 2037; and/or
   (l) a polyadenylation (polyA) signal region, optionally wherein the T2A linker comprises the nucleotide sequence of SEQ ID NO: 2038.
470. The isolated AAV particle of any one of embodiments 459-469, wherein the viral genome comprises in 5′ to 3′ order:
   (i) a 5′ adeno-associated (AAV) ITR, optionally wherein the 5′ AAV ITR comprises the nucleotide sequence of SEQ ID NO: 2026, or a nucleotide sequence at least 95% identical thereto;
   (ii) a CMVie enhancer, optionally wherein the CMVie enhancer comprises the nucleotide sequence of SEQ ID NO: 2027, or a nucleotide sequence at least 95% identical thereto;
   (iii) a CBA promoter or functional variant thereof, optionally wherein the CBA promoter or functional variant thereof comprises the nucleotide sequence of SEQ ID NO: 2028, or a nucleotide sequence at least 95% identical thereto;
   (iv) an intron, optionally wherein the intron comprises the nucleotide sequence of SEQ ID NO: 2029, or a nucleotide sequence at least 95% identical thereto;
   (v) a nucleotide sequence encoding a signal sequence, optionally wherein the nucleotide sequence encoding the signal sequence comprises the nucleotide sequence of SEQ ID NO: 2030, or a nucleotide sequence at least 95% identical thereto;
   (vi) a transgene encoding a heavy chain variable region (VH) of the antibody molecule, wherein the nucleotide sequence encoding the VH comprises the nucleotide sequence of SEQ ID NO: 2031 or a nucleotide sequence at least 80% (e.g., at least 85, 90, 95, 96, 97, 98, or 99%) identical thereto;
   (vii) a nucleotide sequence encoding an IgG1 heavy chain constant region, optionally wherein the nucleotide sequence encoding the IgG1 heavy chain constant region comprises the nucleotide sequence of SEQ ID NO: 2032, or a nucleotide sequence at least 80% (e.g., at least 85, 90, 95, 96, 97, 98, or 99%) identical thereto;
   (viii) a furin cleavage site, optionally wherein the furin cleavage site comprises the nucleotide sequence of SEQ ID NO: 2033, or a nucleotide sequence at least 95% identical thereto;
   (ix) a T2A linker, optionally wherein the T2A linker comprises the nucleotide sequence of SEQ ID NO: 2034, or a nucleotide sequence at least 95% identical thereto;
   (x) a nucleotide sequence encoding a signal sequence, optionally wherein the nucleotide sequence encoding the signal sequence comprises the nucleotide sequence of SEQ ID NO: 2035, or a nucleotide sequence at least 95% identical thereto;
   (xi) a transgene encoding a light chain variable region (VL) of the antibody molecule, wherein the nucleotide sequence encoding the VL comprises the nucleotide sequence of SEQ ID NO: 2036 or a nucleotide sequence at least 80% (e.g., at least 85, 90, 95, 96, 97, 98, or 99%) identical thereto;
   (xii) a nucleotide sequence encoding an IgG kappa light chain constant region, optionally wherein the nucleotide sequence encoding the IgG kappa light chain constant region comprises the nucleotide sequence of SEQ ID NO: 2037, or a nucleotide sequence at least 80% (e.g., at least 85, 90, 95, 96, 97, 98, or 99%) identical thereto;
   (xiii) a polyA signal region, optionally wherein the polyA signal region comprises the nucleotide sequence of SEQ ID NO: 2038, or a nucleotide sequence at least 95% identical thereto; and
   (xiv) a 3′ AAV ITR, optionally wherein the 3′ AAV ITR comprises the nucleotide sequence of SEQ ID NO: 2039, or a nucleotide sequence at least 95% identical thereto.
471. The isolated AAV particle of any one of embodiments 459-470, wherein the viral genome comprises the nucleotide sequence of SEQ ID NO: 2025, or a nucleotide sequence at least 80% (e.g., at least 85, 90, 95, 96, 97, 98, or 99%) identical thereto.
472. The isolated AAV particle of any one of embodiments 459-471, wherein the viral genome further comprises a nucleotide sequence encoding a miR binding site, e.g., a miR binding site that modulates, e.g., reduces, expression of the antibody molecule encoded by the viral genome in a cell or tissue where the corresponding miRNA is expressed.
473. The isolated AAV particle of embodiment 472, wherein the encoded miRNA binding site is complementary, e.g., fully complementary or partially complementary, to a miRNA expressed in a cell or tissue of the DRG, liver, heart, hematopoietic, or a combination thereof.
474. The isolated AAV particle of embodiment 472 or 473, wherein the encoded miR binding site modulates, e.g., reduces, expression of the encoded antibody molecule in a cell or tissue of the DRG, liver, heart, hematopoietic lineage, or a combination thereof.
475. The isolated AAV particle of any one of embodiments 472-474, wherein the viral genome comprises at least 1-5 copies of the encoded miR binding site, e.g., at least 1, 2, 3, 4, or 5 copies.
476. The isolated AAV particle of any one of embodiments 472-475, wherein the viral genome comprises at least 3 copies of an encoded miR binding sites, optionally wherein all three copies comprise the same miR binding site, or at least one, two, three, or all of the copies comprise a different miR binding site.
477. The isolated AAV particle of embodiment 476, wherein the at least 3 copies of the encoded miR binding sites are continuous (e.g., not separated by a spacer), or are separated by a spacer, optionally wherein the spacer comprises the nucleotide sequence GATAGTTA, or a nucleotide sequence having at least one, two, or three modifications, e.g., substitutions (e.g., conservative substitutions), but no more than four modifications, e.g., substitutions (e.g., conservative substitutions), relative to the nucleotide sequence GATAGTTA.
478. The isolated AAV particle of any one of embodiments 472-477, wherein the viral genome comprises at least 4 copies of an encoded miR binding site, optionally wherein all four copies comprise the same miR binding site, or at least one, two, three, or all of the copies comprise a different miR binding site.
479. The isolated AAV particle of embodiment 478, wherein the at least 4 copies of the encoded miR binding sites are continuous (e.g., not separated by a spacer), or are separated by a spacer, optionally wherein the spacer comprises the nucleotide sequence GATAGTTA, or a nucleotide sequence having at least one, two, or three modifications, e.g., substitutions (e.g., conservative substitutions), but no more than four modifications, e.g., substitutions (e.g., conservative substitutions), relative to the nucleotide sequence GATAGTTA.
480. The isolated AAV particle of any one of embodiments 472-479, wherein the encoded miR binding site comprises a miR122 binding site, a miR183 binding site, a miR-1 binding site, a miR-142-3p, or a combination thereof, optionally wherein:
   (i) the encoded miR122 binding site comprises the nucleotide sequence of SEQ ID NO: 3672, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, e.g., substitutions (e.g., conservative substitutions), but no more than ten modifications, e.g., substitutions (e.g., conservative substitutions), relative to SEQ ID NO: 3672;
   (ii) the encoded miR183 binding site comprises the nucleotide sequence of SEQ ID NO: 3675, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, e.g., substitutions (e.g., conservative substitutions), but no more than ten modifications, e.g., substitutions (e.g., conservative substitutions), relative to SEQ ID NO: 3675;
   (iii) the encoded miR-1 binding site comprises the nucleotide sequence of SEQ ID NO: 4679, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, e.g., substitutions (e.g., conservative substitutions), but no more than ten modifications, e.g., substitutions (e.g., conservative substitutions), relative to SEQ ID NO: 4679; and/or
   (iv) the encoded miR-142-3p binding site comprises the nucleotide sequence of SEQ ID NO: 3674, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, e.g., substitutions (e.g., conservative substitutions), but no more than ten modifications, e.g., substitutions (e.g., conservative substitutions), relative to SEQ ID NO: 3674.
481. The isolated AAV particle of any one of embodiments 472-480, wherein the viral genome comprises an encoded miR122 binding site.
482. The isolated AAV particle of any one of embodiments 472-481, wherein the viral genome comprises at least 1-5 copies, e.g., 1, 2, or 3 copies of a miR122 binding site, optionally wherein each copy is continuous (e.g., not separated by a spacer), or each copy is separated by a spacer, optionally wherein the spacer comprises the nucleotide sequence GATAGTTA, or a nucleotide sequence having at least one, two, or three modifications, e.g., substitutions (e.g., conservative substitutions), but no more than four modifications, e.g., substitutions (e.g., conservative substitutions), relative to the nucleotide sequence GATAGTTA.
483. The isolated AAV particle of any one of embodiments 480-482, wherein the encoded miR122 binding site comprises the nucleotide sequence of SEQ ID NO: 3672, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, e.g., substitutions (e.g., conservative substitutions), but no more than ten modifications, e.g., substitutions (e.g., conservative substitutions), relative to SEQ ID NO: 3672.
484. The isolated AAV particle of any one of embodiments 472-483, wherein the viral genome comprises:
(A) (i) a first encoded miR122 binding site comprising the nucleotide sequence of SEQ ID NO: 3672, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, e.g., substitutions (e.g., conservative substitutions), but no more than ten modifications, e.g., substitutions (e.g., conservative substitutions), relative to SEQ ID NO: 3672;
   (ii) a first spacer comprising the nucleotide sequence GATAGTTA, or a nucleotide sequence having at least one, two, or three modifications, e.g., substitutions (e.g., conservative substitutions), but no more than four modifications, e.g., substitutions (e.g., conservative substitutions), relative to the nucleotide sequence GATAGTTA; and
   (iii) a second encoded miR122 binding site comprising the nucleotide sequence of SEQ ID NO: 3672, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, e.g., substitutions (e.g., conservative substitutions), but no more than ten modifications, e.g., substitutions (e.g., conservative substitutions), relative to SEQ ID NO: 3672; or
(B) (i) a first encoded miR122 binding site comprising the nucleotide sequence of SEQ ID NO: 3672, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, e.g., substitutions (e.g., conservative substitutions), but no more than ten modifications, e.g., substitutions (e.g., conservative substitutions), relative to SEQ ID NO: 3672;
   (ii) a first spacer comprising the nucleotide sequence GATAGTTA, or a nucleotide sequence having at least one, two, or three modifications, e.g., substitutions (e.g., conservative substitutions), but no more than four modifications, e.g., substitutions (e.g., conservative substitutions), relative to the nucleotide sequence GATAGTTA;
   (iii) a second encoded miR122 binding site comprising the nucleotide sequence of SEQ ID NO: 3672, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, e.g., substitutions (e.g., conservative substitutions), but no more than ten modifications, e.g., substitutions (e.g., conservative substitutions), relative to SEQ ID NO: 3672;
   (iv) a second spacer comprising the nucleotide sequence GATAGTTA, or a nucleotide sequence having at least one, two, or three modifications, e.g., substitutions (e.g., conservative substitutions), but no more than four modifications, e.g., substitutions (e.g., conservative substitutions), relative to the nucleotide sequence GATAGTTA; and
   (v) a third encoded miR122 binding site comprising the nucleotide sequence of SEQ ID NO: 3672, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, e.g., substitutions (e.g., conservative substitutions), but no more than ten modifications, e.g., substitutions (e.g., conservative substitutions), relative to SEQ ID NO: 3672.
485. The isolated AAV particle of any one of embodiments 472-484, wherein the viral genome comprises an encoded miR183 binding site.
486. The isolated AAV particle of any one of embodiments 472-485, wherein the viral genome comprises at least 1-5 copies, e.g., 1, 2, or 3 copies of a miR183 binding site, optionally wherein each copy is continuous (e.g., not separated by a spacer), or each copy is separated by a spacer, optionally wherein the spacer comprises the nucleotide sequence GATAGTTA, or a nucleotide sequence having at least one, two, or three modifications, e.g., substitutions (e.g., conservative substitutions), but no more than four modifications, e.g., substitutions (e.g., conservative substitutions), relative to the nucleotide sequence GATAGTTA.
487. The isolated AAV particle of embodiment 486, wherein the encoded miR183 binding site comprises the nucleotide sequence of SEQ ID NO: 3675, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, e.g., substitutions (e.g., conservative substitutions), but no more than ten modifications, e.g., substitutions (e.g., conservative substitutions), relative to SEQ ID NO: 3675.
488. The isolated AAV particle of any one of embodiments 472-487, wherein the viral genome comprises:
(A) (i) a first encoded miR183 binding site comprising the nucleotide sequence of SEQ ID NO: 3675, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, e.g., substitutions (e.g., conservative substitutions), but no more than ten modifications, e.g., substitutions (e.g., conservative substitutions), relative to SEQ ID NO: 3675;
   (ii) a first spacer comprising the nucleotide sequence GATAGTTA, or a nucleotide sequence having at least one, two, or three modifications, e.g., substitutions (e.g., conservative substitutions), but no more than four modifications, e.g., substitutions (e.g., conservative substitutions), relative to the nucleotide sequence GATAGTTA; and
   (iii) a second encoded miR183 binding site comprising the nucleotide sequence of SEQ ID NO: 3675, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, e.g., substitutions (e.g., conservative substitutions), but no more than ten modifications, e.g., substitutions (e.g., conservative substitutions), relative to SEQ ID NO: 3675; or
(B) (i) a first encoded miR183 binding site comprising the nucleotide sequence of SEQ ID NO: 3675, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, e.g., substitutions (e.g., conservative substitutions), but no more than ten modifications, e.g., substitutions (e.g., conservative substitutions), relative to SEQ ID NO: 3675;
   (ii) a first spacer comprising the nucleotide sequence GATAGTTA, or a nucleotide sequence having at least one, two, or three modifications, e.g., substitutions (e.g., conservative substitutions), but no more than four modifications, e.g., substitutions (e.g., conservative substitutions), relative to the nucleotide sequence GATAGTTA;
   (iii) a second encoded miR183 binding site comprising the nucleotide sequence of SEQ ID NO: 3675, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, e.g., substitutions (e.g., conservative substitutions), but no more than ten modifications, e.g., substitutions (e.g., conservative substitutions), relative to SEQ ID NO: 3675;
   (iv) a second spacer comprising the nucleotide sequence GATAGTTA, or a nucleotide sequence having at least one, two, or three modifications, e.g., substitutions (e.g., conservative substitutions), but no more than four modifications, e.g., substitutions (e.g., conservative substitutions), relative to the nucleotide sequence GATAGTTA; and
   (v) a third encoded miR183 binding site comprising the nucleotide sequence of SEQ ID NO: 3675, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, e.g., substitutions (e.g., conservative substitutions), but no more than ten modifications, e.g., substitutions (e.g., conservative substitutions), relative to SEQ ID NO: 3675.
489. The isolated AAV particle of any one of embodiments 472-488, wherein the viral genome comprises an encoded miR122 binding site and a miR-1 binding site.
490. The isolated AAV particle of any one of embodiments 472-489, wherein the viral genome is single stranded or self-complementary.
491. The isolated AAV particle of any one of embodiments 472-490, wherein the viral genome further comprises a nucleotide sequence encoding a Rep protein, e.g., a non-structural protein, wherein the Rep protein comprises a Rep78 protein, a Rep68, Rep52 protein, and/or a Rep40 protein.
492. The isolated AAV particle of embodiment 491, wherein the Rep78 protein, the Rep68 protein, the Rep52 protein, and/or the Rep40 protein are encoded by at least one Rep gene.
493. A cell, e.g., a host cell, comprising the AAV particle of any one of the preceding embodiments.
494. The cell of embodiment 493, wherein the cell is a mammalian cell or an insect cell.
495. The cell of embodiment 493 or 494, wherein the cell is a cell of a brain region or a spinal cord region, optionally a cell of the frontal cortex, sensory cortex, motor cortex, caudate, dentate nucleus, cerebellar cortex, cerebral cortex, brain stem, hippocampus, thalamus, putamen, cervical spinal cord region, thoracic spinal cord region, and/or lumbar spinal cord region.
496. The cell of any one of embodiments 493-495, wherein the cell is a neuron, a sensory neuron, a motor neuron, an astrocyte, or a glial cell.
497. A method of making the AAV particle of any one of embodiments 1-492, comprising
   (i) providing a host cell comprising a viral genome; and
   (ii) incubating the host cell under conditions suitable to enclose the viral genome in an AAV capsid variant, e.g., an AAV capsid variant described herein;
   thereby making the AAV particle.
498. The method of embodiment 497, further comprising, prior to step (i), introducing a first nucleic acid molecule comprising the viral genome into the host cell.
499. The method of embodiment 497 or 498, wherein the host cell comprises a second nucleic acid encoding the capsid variant.
500. The method of embodiment 499, wherein the second nucleic acid molecule is introduced into the host cell prior to, concurrently with, or after the first nucleic acid molecule.
501. A pharmaceutical composition comprising the AAV particle of any one of embodiments 1-492, and a pharmaceutically acceptable excipient.
502. A method of delivering a payload to a cell or tissue (e.g., a CNS cell or a CNS tissue), comprising administering an effective amount of the pharmaceutical composition of embodiment 501, or the AAV particle of any one of embodiments 1-492.
503. The method of embodiment 502, wherein the cell is a cell a cell of a brain region or a spinal cord region, optionally a cell of the frontal cortex, sensory cortex, motor cortex, caudate, dentate nucleus, cerebellar cortex, cerebral cortex, brain stem, hippocampus, thalamus, putamen, cervical spinal cord region, thoracic spinal cord region, and/or lumbar spinal cord region.
504. The method of embodiment 502 or 503, wherein the cell is a neuron, a sensory neuron, a motor neuron, or an astrocyte.
505. The method of any one of embodiments 502-504, wherein the cell or tissue is within a subject.
506. The method of embodiment 505, wherein the subject has, has been diagnosed with having, or is at risk of having a neurological, e.g., a neurodegenerative disorder.
507. The method of embodiment 505 or 506, wherein the subject has, has been diagnosed with having, or is at risk of having a disease associated with tau expression or activity.
508. The method of any one of embodiments 505-507, wherein the subject has, has been diagnosed with having, or is at risk of having a tau-related disease (e.g., a tauopathy).
509. The method of any one of embodiments 505-508, wherein the subject has, has been diagnosed with having, or is at risk of having Alzheimer's disease (AD), frontotemporal dementia and parkinsonism linked to chromosome 17 (FTDP-17), frontotemporal lobar degeneration (FTLD), frontotemporal dementia (FTD), chronic traumatic encephalopathy (CTE), progressive Supranuclear Palsy (PSP), Down's syndrome, Pick's disease, corticobasal degeneration (CBD), corticobasal syndrome, amyotrophic lateral sclerosis (ALS), prion diseases, Creutzfeldt-Jakob disease (CJD), multiple system atrophy, tangle-only dementia, or progressive subcortical gliosis.
510. A method of treating a subject having or diagnosed with having a neurological disorder, e.g., a neurodegenerative disorder, comprising administering to the subject an effective amount of the pharmaceutical composition of embodiment 501, or the AAV particle of any one of embodiments 1-492.
511. A method of treating a subject having or diagnosed with having a disease related to expression of tau expression, comprising administering to the subject an effective amount of the pharmaceutical composition of embodiment 501, or the AAV particle of any one of embodiments 1-492.
512. A method of treating a subject having or diagnosed with a tauopathy, comprising administering to the subject an effective amount of the pharmaceutical composition of embodiment 501, or the AAV particle of any one of embodiments 1-492.
513. The method of any one of embodiments 506-512, wherein the neurological disorder, neurodegenerative disorder, disease associated with tau, or tauopathy is Alzheimer's Disease, frontotemporal dementia and parkinsonism linked to chromosome 17 (FTDP-17), frontotemporal lobar degeneration (FTLD), frontotemporal dementia (FTD), chronic traumatic encephalopathy (CTE), progressive Supranuclear Palsy (PSP), Down's syndrome, Pick's disease, corticobasal degeneration (CBD), corticobasal syndrome, amyotrophic lateral sclerosis (ALS), prion diseases, Creutzfeldt-Jakob disease (CJD), multiple system atrophy, tangle-only dementia, or progressive subcortical gliosis.
514. The method of any one of embodiments 510-513, where treating comprises prevention of progression of the disease or disorder in the subject.
515. The method of any one of embodiments 505-514, wherein the subject is a human.
516. The method of any one of embodiments 505-515, wherein the AAV particle is administered to the subject intravenously, via intra-cisterna magna injection (ICM), intracerebrally, intrathecally, intracerebroventricularly, via intraparenchymal administration, or intramuscularly.
517. The method of any one of embodiments 505-516, wherein the AAV particle is administered to the subject via focused ultrasound (FUS), e.g., coupled with the intravenous administration of microbubbles (FUS-MB), or MRI-guided FUS coupled with intravenous administration.
518. The method of any one of embodiments 505-517, wherein the AAV particle is administered to the subject intravenously.
519. The method of any one of embodiments 502-518, wherein administration of the AAV particle results in a decreased presence, level, and/or activity of tau mRNA, tau protein, or combination thereof.
520. The method of any one of embodiments 502-519, wherein administration of the AAV particle results in an increased presence, level, and/or activity of tau mRNA, tau protein, or a combination thereof.
521. The pharmaceutical composition of embodiment 501, or the AAV particle of any one of embodiments 1-492, for use in a method of delivering a payload to a cell or tissue.
522. The pharmaceutical composition of embodiment 501, or the AAV particle of any one of embodiments 1-492, for use in a method of treating a neurological disorder, a neurodegenerative disorder, a disease associated with tau expression or activity, or a tauopathy.
523. The pharmaceutical composition of embodiment 501, or the AAV particle of any one of embodiments 1-492, for use in the manufacture of a medicament.
524. Use of the pharmaceutical composition of embodiment 501, or the AAV particle of any one of embodiments 1-492, in the manufacture of a medicament.
525. Use of the pharmaceutical composition of embodiment 501, or the AAV particle of any one of embodiments 1-492, in the manufacture of a medicament for treating a neurological disorder, a neurodegenerative disorder, a disease associated with tau expression or activity, or a tauopathy.

### BRIEF DESCRIPTION OF THE DRAWINGS

FIG. 1A is a graph showing the percentage of transduced cells (% HA positive cells) in various brain regions of NHPs following intravenous administration of a dose of 6.7e12 VG/kg of AAV particles comprising the TTD-001 capsid variant. FIG. 1B is a graph showing the percentage of transduced cells (% HA positive cells) in various brain regions of NHPs following intravenous administration of a dose of 2e13 VG/kg of AAV particles comprising the TTD-001 capsid variant. FIG. 1C is a graph showing the percentage of neuronal transduction (% HA cells among SMI311+cells) in the thalamus, dentate nucleus, and spinal cord of the NHPs following intravenous administration of a dose of 2e13 VG/kg of AAV particles comprising the TTD-001 capsid variant.

### DETAILED DESCRIPTION

### Overview

Described herein, inter alia, are compositions comprising isolated, e.g., recombinant, viral particles, e.g., AAV particles, for delivering functional anti-tau antibodies, and methods of making and using the same. Adeno-associated viruses (AAV) are small non-enveloped icosahedral capsid viruses of the Parvoviridae family characterized by a single stranded DNA viral genome. Parvoviridae family viruses consist of two subfamilies: Parvovirinae, which infect vertebrates, and Densovirinae, which infect invertebrates. The Parvoviridae family includes the Dependovirus genus which includes AAV, capable of replication in vertebrate hosts including, but not limited to, human, primate, bovine, canine, equine, and ovine species.

The parvoviruses and other members of the Parvoviridae family are generally described in Kenneth I. Berns, “Parvoviridae: The Viruses and Their Replication,” Chapter 69 in Fields Virology (3d Ed. 1996), the contents of which are incorporated by reference in their entirety.

AAV have proven to be useful as a biological tool due to their relatively simple structure, their ability to infect a wide range of cells (including quiescent and dividing cells) without integration into the host genome and without replicating, and their relatively benign immunogenic profile. The genome of the virus may be manipulated to contain a minimum of components for the assembly of a functional recombinant virus, or viral particle, which is loaded with or engineered to target a particular tissue and express or deliver a desired payload. The genome of the virus may be modified to contain a minimum of components for the assembly of a functional recombinant virus, or viral particle, which is loaded with or engineered to express or deliver a desired nucleic acid construct or payload, e.g., a transgene, polypeptide-encoding polynucleotide, e.g., an anti-tau antibody molecule, which may be delivered to a target cell, tissue, or organism. In some embodiments, the target cell is a CNS cell. In some embodiments, the target tissue is a CNS tissue. The target CNS tissue may be brain tissue. In some embodiments, the brain target comprises caudate, putamen, thalamus, superior colliculus, cortex, and corpus collosum.

Gene therapy presents an alternative approach tauopathies and related disorders. AAVs are commonly used in gene therapy approaches as a result of a number of advantageous features. Without wishing to be bound by theory, it is believed in some embodiments, that expression vectors, e.g., an adeno-associated viral vector (AAVs) or AAV particle, e.g., an AAV particle described herein, can be used to administer and/or deliver an anti-tau antibody molecule in order to achieve sustained, high concentrations, allowing for longer lasting efficacy, fewer dose treatments, broad biodistribution, and/or more consistent levels of the antibody, relative to a non-AAV therapy.

As demonstrated in the Examples herein below, certain AAV capsid variants described herein show multiple advantages over wild-type AAV9, including (i) increased penetrance through the blood brain barrier following intravenous administration, (ii) wider distribution throughout the multiple brain regions, e.g., frontal cortex, sensory cortex, motor cortex, putamen, thalamus, cerebellar cortex, dentate nucleus, caudate, and/or hippocampus, and/or (iii) elevated payload expression in multiple brain regions. Without wishing to be being bound by theory, it is believed that these advantages may be due, in part, to the dissemination of the AAV capsid variants through the brain vasculature. In some embodiments, the AAV capsids described herein enhance the delivery of a payload, e.g., an anti-tau antibody molecule described herein, to multiple regions of the brain including for example, the frontal cortex, sensory cortex, motor cortex, putamen, thalamus, cerebellar cortex, dentate nucleus, caudate, and/or hippocampus. In some embodiments, enhance the expression of a payload, e.g., an anti-tau antibody described herein, to multiple cell types in the CNS, e.g., neurons, oligodendrocytes, and/or glial cells. Without wishing to be bound by theory, an AAV particle comprising an AAV capsid polypeptide, e.g., an AAV capsid variant described herein, for the vectorized delivery of an anti-tau antibody described here will result in increased penetrance through the blood brain barrier, e.g., following intravenous administration, and/or increased biodistribution of the anti-tau antibody in the central nervous system, e.g., the brain and the spinal cord.

### I. Compositions

According to the present disclosure, compositions for delivering functional anti-tau antibody molecules by adeno-associated virus particles (AAVs) are provided. In some embodiments, an AAV particle, e.g., an AAV particle as described herein, or plurality of particles, may be provided, e.g., delivered, via any of several routes of administration, to a cell, tissue, organ, or organism, in vivo, ex vivo, or in vitro.

In some embodiments, AAV particles, nucleic acids, e.g., nucleic acid molecules encoding an antibody molecule, and/or payloads, e.g., an antibody molecule, and methods of using and making the same are described in WO2017189963, the contents of which are herein incorporated by reference in their entirety.

In some embodiments, the nucleic acid sequences, genetic elements, AAV vectors, and polypeptides disclosed herein may be engineered to contain modular elements and/or sequence motifs assembled to enable expression of an antibody molecule, e.g., an antibody molecule described herein. In some embodiments, the genetic element comprises a nucleotide sequence encoding a transgene encoding an antibody molecule (e.g., an antibody molecule described herein). In some embodiments, the nucleic acid sequence encodes an antibody molecule comprising one or more of the CDRs (e.g., heavy chain and/or light chain CDRs) of an antibody molecule, a variable heavy (VH) chain region and/or variable light (VL) chain region, a heavy and/or light chain constant region, a heavy and/or light chain, or a combination thereof. In some embodiments, the nucleic acid sequence encoding the antibody molecule may also encode a linker, e.g., such that the VH/heavy chain and the VL/light chain of the encoded antibody molecule are connected via a linker. In some embodiments, the order of expression, structural position, or concatemer count (e.g., the VH, VL, heavy chain, light chain, and/or linker) may be different within or among genetic element sequences. In some embodiments, the identity, position, and number of linkers expressed by a genetic element described herein may vary. In some embodiments, the genetic element may further comprise an internal repeat (ITR) sequence, promoter region, an intron region, an exon region, a Kozak sequence, an enhancer, a polyadenylation sequence, or combination thereof.

In some embodiments, the present disclosure provides methods for delivering an antibody molecule (e.g., an anti-tau antibody described herein) and/or a nucleic acid sequence encoding an antibody molecule (e.g., an anti-tau antibody described herein) comprised within the genetic element comprised within a recombinant, AAV particle (e.g., an AAV particle described herein) to a cell, tissue, organ, or subject.

### Adeno-Associated Viral (AAV) Particles

AAV have a genome of about 5,000 nucleotides in length which contains two open reading frames encoding the proteins responsible for replication (Rep) and the structural protein of the capsid (Cap). The open reading frames are flanked by two Inverted Terminal Repeat (ITR) sequences, which serve as the origin of replication of the viral genome. The wild-type AAV viral genome comprises nucleotide sequences for two open reading frames, one for the four non-structural Rep proteins (Rep78, Rep68, Rep52, Rep40, encoded by Rep genes) and one for the three capsid, or structural, proteins (VP1, VP2, VP3, encoded by capsid genes or Cap genes). The Rep proteins are important for replication and packaging, while the capsid proteins are assembled to create the protein shell of the AAV, or AAV capsid. Alternative splicing and alternate initiation codons and promoters result in the generation of four different Rep proteins from a single open reading frame and the generation of three capsid proteins from a single open reading frame. Though it varies by AAV serotype, as a non-limiting example, for AAV9/hu.14 (SEQ ID NO: 123 of U.S. Pat. No. 7,906,111, the contents of which are herein incorporated by reference in their entirety) VP1 refers to amino acids 1-736, VP2 refers to amino acids 138-736, and VP3 refers to amino acids 203-736. As another non-limiting example, VP1 refers to amino acids 1-743 numbered according to SEQ ID NO: 1, VP2 refers to amino acids 138-743 numbered according to SEQ ID NO: 1, and VP3 refers to amino acids 203-743 numbered according to SEQ ID NO: 1. In other words, VP1 is the full-length capsid sequence, while VP2 and VP3 are shorter components of the whole. As a result, changes in the sequence in the VP3 region, are also changes to VP1 and VP2, however, the percent difference as compared to the parent sequence will be greatest for VP3 since it is the shortest sequence of the three. Though described here in relation to the amino acid sequence, the nucleic acid sequence encoding these proteins can be similarly described. Together, the three capsid proteins assemble to create the AAV capsid protein. While not wishing to be bound by theory, the AAV capsid protein typically comprises a molar ratio of 1:1:10 of VP1:VP2:VP3. As used herein, an “AAV serotype” is defined primarily by the AAV capsid. In some instances, the ITRs are also specifically described by the AAV serotype (e.g., AAV2/9).

The AAV particle typically requires a co-helper (e.g., adenovirus) to undergo productive infection in cells. In the absence of such helper functions, the AAV virions essentially enter host cells but do not integrate into the cells' genome.

AAV particles have been investigated for delivery of gene therapeutics because of several unique features. Non-limiting examples of the features include (i) the ability to infect both dividing and non-dividing cells; (ii) a broad host range for infectivity, including human cells; (iii) wild-type AAV has not been associated with any disease and has not been shown to replicate in infected cells; (iv) the lack of cell-mediated immune response against the vector, and (v) the non-integrative nature in a host chromosome thereby reducing potential for long-term genetic alterations. Moreover, infection with AAV vectors has minimal influence on changing the pattern of cellular gene expression (Stilwell and Samulski et al., Biotechniques, 2003, 34, 148, the contents of which are herein incorporated by reference in their entirety).

Typically, AAV particles for anti-tau antibody molecule delivery may comprise recombinant viral genomes which are replication defective as they lack sequences encoding functional Rep and Cap proteins within the viral genome. In some cases, the defective AAV genomes may lack most or all coding sequences and essentially only contain one or two AAV ITR sequences and a payload sequence. In certain embodiments, the viral genome encodes an anti-tau antibody molecule.

In some embodiments, the AAV particles of the present disclosure may be introduced into mammalian cells.

AAV particles may be modified to enhance the efficiency of delivery. Such modified AAV particles of the present disclosure can be packaged efficiently and can be used to successfully infect the target cells at high frequency and with minimal toxicity.

In other embodiments, AAV particles of the present disclosure may be used to deliver an anti-tau antibody molecule to the central nervous system (see, e.g., U.S. Pat. No. 6,180,613; the contents of which are herein incorporated by reference in their entirety) or to specific tissues of the CNS.

It is understood that the compositions described herein may have additional conservative or non-essential amino acid substitutions, which do not have a substantial effect on their functions.

### AAV Capsids and Variants Thereof

In some embodiments, an AAV particle, e.g., an AAV particle for the vectorized delivery of protein described herein (e.g., an anti-tau antibody molecule), may comprise an AAV capsid polypeptide, e.g., an AAV capsid variant.

In some embodiments, the AAV capsid polypeptide, e.g., AAV capsid variant, allows for blood brain barrier penetration following intravenous administration. In some embodiments, the AAV capsid, e.g., AAV capsid variant, allows for blood brain barrier penetration following focused ultrasound (FUS), e.g., coupled with the intravenous administration of microbubbles (FUS-MB), or MRI-guided FUS coupled with intravenous administration. In some embodiments the AAV capsid, e.g., AAV capsid variant allows for increased distribution to a brain region. In some embodiments, the brain region comprises a frontal cortex, sensory cortex, motor cortex, caudate, dentate nucleus, cerebellar cortex, cerebral cortex, brain stem, hippocampus, thalamus, putamen, or a combination thereof. In some embodiments, the AAV capsid, e.g., AAV capsid variant allows for preferential transduction in a brain region relative to the transduction in the dorsal root ganglia (DRG).

In some embodiments the AAV capsid polypeptide, e.g., AAV capsid variant allows for increased distribution to a spinal cord region. In some embodiments, the spinal region comprises a cervical spinal cord region, thoracic spinal cord region, and/or lumbar spinal cord region.

In some embodiments, the AAV capsid polypeptide, e.g., an AAV capsid variant comprises a VOY101 capsid polypeptide, an AAVPHP.B (PHP.B) capsid polypeptide, a AAVPHP.N (PHP.N) capsid polypeptide, an AAV1 capsid polypeptide, an AAV2 capsid polypeptide, an AAVS capsid polypeptide, an AAV9 capsid polypeptide, an AAV9 K449R capsid polypeptide, an AAVrh10 capsid polypeptide, or a functional variant thereof. In some embodiments, the AAV capsid polypeptide, e.g., AAV capsid variant, comprises an amino acid sequence of any of the AAV capsid polypeptides in Table 1, or an amino acid sequence substantially identical (e.g., having at least 70%, 75%, 80%, 85%, 90%, 92%, 95%, 97%, 98%, or 99% sequence identity) thereto. In some embodiments, the nucleotide sequence encoding the AAV capsid polypeptide comprises any one of the nucleotide sequences in Table 1, or a nucleotide sequence substantially identical (e.g., having at least 70%, 75%, 80%, 85%, 90%, 92%, 95%, 97%, 98%, or 99% sequence identity) thereto.

In some embodiments, the AAV capsid variant, described herein does not comprise an amino acid sequence present immediately subsequent to position 586, 588, or 589 numbered relative to SEQ ID NO: 138, having at least 5 consecutive amino acids corresponding to positions 586 to 599, e.g., 586 to 594, 587 to 595, 588 to 596, 589 to 597, 590 to 598, numbered relative to the amino acid sequences in Table 1 of WO2020223276, the contents of which are hereby incorporated by reference in their entirety.

In any of the embodiments described herein, a position comprising 5 consecutive amino acids corresponding to positions 586 to 599, e.g., 586 to 594, 587 to 595, 588 to 596, 589 to 597, 590 to 598 numbered relative to SEQ ID NO: 138 can be identified by providing an alignment of a reference sequence and a query sequence, wherein the reference sequence is SEQ ID NO: 138, and identifying the residues corresponding to the positions in the query sequence that correspond to positions 586 to 599, e.g., 586 to 594, 587 to 595, 588 to 596, 589 to 597, 590 to 598 in the reference sequence.

In some embodiments, the AAV capsid polypeptide, e.g., AAV capsid variant, described herein does not comprise an amino acid sequence present immediately subsequent to position 586, 588, or 589 numbered relative to SEQ ID NO: 138, at least 5 consecutive amino acids corresponding to positions 586 to 599, e.g., 586 to 594, 587 to 595, 588 to 596, 589 to 597, 590 to 598, numbered relative to SEQ ID NO: 138. In some embodiments, the AAV capsid polypeptide, e.g., AAV capsid variant, described herein, does not comprise an amino acid sequence present immediately subsequent to position 586, 588, or 589 numbered relative to SEQ ID NO: 138, having at least 5 consecutive amino acids corresponding to positions 586 to 599, e.g., 586 to 594, 587 to 595, 588 to 596, 589 to 597, 590 to 598, numbered relative to SEQ ID NO: 12. In some embodiments, the AAV capsid polypeptide, e.g., AAV capsid variant, described herein, does not comprise an amino acid sequence present immediately subsequent to position 586, 588, or 589 numbered relative to SEQ ID NO: 138, having at least than 5 consecutive amino acids corresponding to positions 586 to 599, e.g., 586 to 594, 587 to 595, 588 to 596, 589 to 597, 590 to 598, numbered relative to SEQ ID NO: 13. In some embodiments, the AAV capsid polypeptide, e.g., AAV capsid variant, or the parent AAV capsid may be, at a position other than 5 consecutive amino acids corresponding to positions 586 to 599, e.g., 586 to 594, 587 to 595, 588 to 596, 589 to 597, 590 to 598, numbered relative to SEQ ID NO: 1.

TABLE 1
Exemplary full length capsid sequences

| | SEQ | |
|---|---|---|
| | ID | |
| Description | NO: | Sequence Information |
| VOY101 | 1 | MAADGYLPDWLEDNLSEGIREWWALKPGAPQPKANQQHQDNARGLVLPGYKYLG |
| | | PGNGLDKGEPVNAADAAALEHDKAYDQQLKAGDNPYLKYNHADAEFQERLKEDT |
| | | SFGGNLGRAVFQAKKRLLEPLGLVEEAAKTAPGKKRPVEQSPQEPDSSAGIGKS |
| | | GAQPAKKRLNFGQTGDTESVPDPQPIGEPPAAPSGVGSLTMASGGGAPVADNNE |
| | | GADGVGSSSGNWHCDSQWLGDRVITTSTRIWALPTYNNHLYKQISNSTSGGSSN |
| | | DNAYFGYSTPWGYFDFNRFHCHFSPRDWQRLINNNWGFRPKRLNFKLFNIQVKE |
| | | VTDNNGVKTIANNLTSTVQVFTDSDYQLPYVLGSAHEGCLPPFPADVFMIPQYG |
| | | YLTLNDGSQAVGRSSFYCLEYFPSQMLRTGNNFQFSYEFENVPFHSSYAHSQSL |
| | | DRLMNPLIDQYLYYLSKIINGSGQNQQTLKFSVAGPSNMAVQGRNYIPGPSYRQ |
| | | QRVSTTVTQNNNSEFAWPGASSWALNGRNSLMNPGPAMASHKEGEDRFFPLSGS |
| | | LIFGKQGTGRDNVDADKVMITNEEEIKTTNPVATESYGQVATNHQSDGTLAVPF |
| | | KAQAQTGWVQNQGILPGMVWQDRDVYLQGPIWAKIPHTDGNFHPSPLMGGFGMK |
| | | HPPPQILIKNTPVPADPPTAFNKDKLNSFITQYSTGQVSVEIEWELQKENSKRW |
| | | NPEIQYTSNYYKSNNVEFAVNTEGVYSEPRPIGTRYLTRNL |
| AAV9/hu.1 | 11 | MAADGYLPDWLEDNLSEGIREWWALKPGAPQPKANQQHQDNARGLVLPGYKYLG |
| 4 K449R | | PGNGLDKGEPVNAADAAALEHDKAYDQQLKAGDNPYLKYNHADAEFQERLKEDT |
| | | SFGGNLGRAVFQAKKRLLEPLGLVEEAAKTAPGKKRPVEQSPQEPDSSAGIGKS |
| | | GAQPAKKRLNFGQTGDTESVPDPQPIGEPPAAPSGVGSLTMASGGGAPVADNNE |
| | | GADGVGSSSGNWHCDSQWLGDRVITTSTRIWALPTYNNHLYKQISNSTSGGSSN |
| | | DNAYFGYSTPWGYFDFNRFHCHFSPRDWQRLINNNWGFRPKRLNFKLFNIQVKE |
| | | VTDNNGVKTIANNLTSTVQVFTDSDYQLPYVLGSAHEGCLPPFPADVEMIPQYG |
| | | YLTLNDGSQAVGRSSFYCLEYFPSQMLRTGNNFQFSYEFENVPFHSSYAHSQSL |
| | | DRLMNPLIDQYLYYLSRTINGSGQNQQTLKFSVAGPSNMAVQGRNYIPGPSYRQ |
| | | QRVSTTVTQNNNSEFAWPGASSWALNGRNSLMNPGPAMASHKEGEDRFFPLSGS |
| | | LIFGKQGTGRDNVDADKVMITNEEEIKTTNPVATESYGQVATNHQSAQAQAQTG |
| | | WVQNQGILPGMVWQDRDVYLQGPIWAKIPHTDGNFHPSPLMGGFGMKHPPPQIL |
| | | IKNTPVPADPPTAFNKDKLNSFITQYSTGQVSVEIEWELQKENSKRWNPEIQYT |
| | | SNYYKSNNVEFAVNTEGVYSEPRPIGTRYLTRNL |
| AAV9/hu.1 | 138 | MAADGYLPDWLEDNLSEGIREWWALKPGAPQPKANQQHQDNARGLVLPGYKYLG |
| 4 WT | | PGNGLDKGEPVNAADAAALEHDKAYDQQLKAGDNPYLKYNHADAEFQERLKEDT |
| (amino | | SFGGNLGRAVFQAKKRLLEPLGLVEEAAKTAPGKKRPVEQSPQEPDSSAGIGKS |
| acid) | | GAQPAKKRLNFGQTGDTESVPDPQPIGEPPAAPSGVGSLIMASGGGAPVADNNE |
| | | GADGVGSSSGNWHCDSQWLGDRVITTSTRTWALPTYNNHLYKQISNSTSGGSSN |
| | | DNAYFGYSTPWGYFDFNRFHCHFSPRDWQRLINNNWGFRPKRLNFKLFNIQVKE |
| | | VTDNNGVKTIANNLTSTVQVFTDSDYQLPYVLGSAHEGCLPPFPADVFMIPQYG |
| | | YLTLNDGSQAVGRSSFYCLEYFPSQMLRTGNNFQFSYEFENVPFHSSYAHSQSL |
| | | DRLMNPLIDQYLYYLSKTINGSGQNQQTLKFSVAGPSNMAVQGRNYIPGPSYRQ |
| | | QRVSTTVTQNNNSEFAWPGASSWALNGRNSLMNPGPAMASHKEGEDRFFPLSGS |
| | | LIFGKQGTGRDNVDADKVMITNEEEIKTINPVATESYGQVATNHQSAQAQAQTG |
| | | WVQNQGILPGMVWQDRDVYLQGPIWAKIPHTDGNFHPSPLMGGFGMKHPPPQIL |
| | | IKNTPVPADPPTAFNKDKLNSFITQYSTGQVSVEIEWELQKENSKRWNPEIQYT |
| | | SNYYKSNNVEFAVNTEGVYSEPRPIGTRYLTRNL |
| | | ATGGCTGCCGATGGTTATCTTCCAGATTGGCTCGAGGACAACCTTAGTGAAGGA |
| AAV9/hu.1 | 137 | ATTCGCGAGTGGTGGGCTTTGAAACCTGGAGCCCCTCAACCCAAGGCAAATCAA |
| 4 WT | | CAACATCAAGACAACGCTCGAGGTCTTGTGCTTCCGGGTTACAAATACCTTGGA |
| (DNA) | | CCCGGCAACGGACTCGACAAGGGGGAGCCGGTCAACGCAGCAGACGCGGCGGCC |
| | | CTCGAGCACGACAAGGCCTACGACCAGCAGCTCAAGGCCGGAGACAACCCGTAC |
| | | CTCAAGTACAACCACGCCGACGCCGAGTTCCAGGAGCGGCTCAAAGAAGATACG |
| | | TCTTTTGGGGGCAACCTCGGGCGAGCAGTCTTCCAGGCCAAAAAGAGGCTTCTT |
| | | GAACCTCTTGGTCTGGTTGAGGAAGCGGCTAAGACGGCTCCTGGAAAGAAGAGG |
| | | CCTGTAGAGCAGTCTCCTCAGGAACCGGACTCCTCCGCGGGTATTGGCAAATCG |
| | | GGTGCACAGCCCGCTAAAAAGAGACTCAATTTCGGTCAGACTGGCGACACAGAG |
| | | TCAGTCCCAGACCCTCAACCAATCGGAGAACCTCCCGCAGCCCCCTCAGGTGTG |
| | | GGATCTCTTACAATGGCTTCAGGTGGTGGCGCACCAGTGGCAGACAATAACGAA |
| | | GGTGCCGATGGAGTGGGTAGTTCCTCGGGAAATTGGCATTGCGATTCCCAATGG |
| | | CTGGGGGACAGAGTCATCACCACCAGCACCCGAACCTGGGCCCTGCCCACCTAC |
| | | AACAATCACCTCTACAAGCAAATCTCCAACAGCACATCTGGAGGATCTTCAAAT |
| | | GACAACGCCTACTTCGGCTACAGCACCCCCTGGGGGTATTTTGACTTCAACAGA |
| | | TTCCACTGCCACTTCTCACCACGTGACTGGCAGCGACTCATCAACAACAACTGG |
| | | GGATTCCGGCCTAAGCGACTCAACTTCAAGCTCTTCAACATTCAGGTCAAAGAG |
| | | GTTACGGACAACAATGGAGTCAAGACCATCGCCAATAACCTTACCAGCACGGTC |
| | | CAGGTCTTCACGGACTCAGACTATCAGCTCCCGTACGTGCTCGGGTCGGCTCAC |
| | | GAGGGCTGCCTCCCGCCGTTCCCAGCGGACGTTTTCATGATTCCTCAGTACGGG |
| | | TATCTGACGCTTAATGATGGAAGCCAGGCCGTGGGTCGTTCGTCCTTTTACTGC |
| | | CTGGAATATTTCCCGTCGCAAATGCTAAGAACGGGTAACAACTTCCAGTTCAGC |
| | | TACGAGTTTGAGAACGTACCTTTCCATAGCAGCTACGCTCACAGCCAAAGCCTG |
| | | GACCGACTAATGAATCCACTCATCGACCAATACTTGTACTATCTCTCAAAGACT |
| | | ATTAACGGTTCTGGACAGAATCAACAAACGCTAAAATTCAGTGTGGCCGGACCC |
| | | AGCAACATGGCTGTCCAGGGAAGAAACTACATACCTGGACCCAGCTACCGACAA |
| | | CAACGTGTCTCAACCACTGTGACTCAAAACAACAACAGCGAATTTGCTTGGCCT |
| | | GGAGCTTCTTCTTGGGCTCTCAATGGACGTAATAGCTTGATGAATCCTGGACCT |
| | | GCTATGGCCAGCCACAAAGAAGGAGAGGACCGTTTCTTTCCTTTGTCTGGATCT |
| | | TTAATTTTTGGCAAACAAGGAACTGGAAGAGACAACGTGGATGCGGACAAAGTC |
| | | ATGATAACCAACGAAGAAGAAATTAAAACTACTAACCCGGTAGCAACGGAGTCC |
| | | TATGGACAAGTGGCCACAAACCACCAGAGTGCCCAAGCACAGGCGCAGACCGGC |
| | | TGGGTTCAAAACCAAGGAATACTTCCGGGTATGGTTTGGCAGGACAGAGATGTG |
| | | TACCTGCAAGGACCCATTTGGGCCAAAATTCCTCACACGGACGGCAACTTTCAC |
| | | CCTTCTCCGCTGATGGGAGGGTTTGGAATGAAGCACCCGCCTCCTCAGATCCTC |
| | | ATCAAAAACACACCTGTACCTGCGGATCCTCCAACGGCCTTCAACAAGGACAAG |
| | | CTGAACTCTTTCATCACCCAGTATTCTACTGGCCAAGTCAGCGTGGAGATCGAG |
| | | TGGGAGCTGCAGAAGGAAAACAGCAAGCGCTGGAACCCGGAGATCCAGTACACT |
| | | TCCAACTATTACAAGTCTAATAATGTTGAATTTGCTGTTAATACTGAAGGTGTA |
| | | TATAGTGAACCCCGCCCCATTGGCACCAGATACCTGACTCGTAATCTGTAA |
| PHP.B | 12 | MAADGYLPDWLEDNLSEGIREWWALKPGAPQPKANQQHQDNARGLVLPGYKYLG |
| (amino | | PGNGLDKGEPVNAADAAALEHDKAYDQQLKAGDNPYLKYNHADAEFQERLKEDT |
| acid) | | SFGGNLGRAVFQAKKRLLEPLGLVEEAAKTAPGKKRPVEQSPQEPDSSAGIGKS |
| | | GAQPAKKRLNFGQTGDTESVPDPQPIGEPPAAPSGVGSLTMASGGGAPVADNNE |
| | | GADGVGSSSGNWHCDSQWLGDRVITTSTRTWALPTYNNHLYKQISNSTSGGSSN |
| | | DNAYFGYSTPWGYFDFNRFHCHFSPRDWQRLINNNWGFRPKRLNFKLFNIQVKE |
| | | VTDNNGVKTIANNLTSTVQVFTDSDYQLPYVLGSAHEGCLPPFPADVFMIPQYG |
| | | YLTLNDGSQAVGRSSFYCLEYFPSQMLRTGNNFQFSYEFENVPFHSSYAHSQSL |
| | | DRLMNPLIDQYLYYLSRTINGSGQNQQTLKFSVAGPSNMAVQGRNYIPGPSYRQ |
| | | QRVSTTVTQNNNSEFAWPGASSWALNGRNSLMNPGPAMASHKEGEDRFFPLSGS |
| | | LIFGKQGTGRDNVDADKVMITNEEEIKTTNPVATESYGQVATNHQSAQTLAVPF |
| | | KAQAQTGWVQNQGILPGMVWQDRDVYLQGPIWAKIPHTDGNFHPSPLMGGFGMK |
| | | HPPPQILIKNTPVPADPPTAFNKDKLNSFITQYSTGQVSVEIEWELQKENSKRW |
| | | NPEIQYTSNYYKSNNVEFAVNTEGVYSEPRPIGTRYLTRNL |
| PHP.N | 13 | MAADGYLPDWLEDNLSEGIREWWALKPGAPQPKANQQHQDNARGLVLPGYKYLG |
| (amino | | PGNGLDKGEPVNAADAAALEHDKAYDQQLKAGDNPYLKYNHADAEFQERLKEDT |
| acid) | | SFGGNLGRAVFQAKKRLLEPLGLVEEAAKTAPGKKRPVEQSPQEPDSSAGIGKS |
| | | GAQPAKKRLNFGQTGDTESVPDPQPIGEPPAAPSGVGSLTMASGGGAPVADNNE |
| | | GADGVGSSSGNWHCDSQWLGDRVITTSTRIWALPTYNNHLYKQISNSTSGGSSN |
| | | DNAYFGYSTPWGYFDFNRFHCHFSPRDWQRLINNNWGFRPKRLNFKLFNIQVKE |
| | | VTDNNGVKTIANNLTSTVQVFTDSDYQLPYVLGSAHEGCLPPFPADVFMIPQYG |
| | | YLTLNDGSQAVGRSSFYCLEYFPSQMLRTGNNFQFSYEFENVPFHSSYAHSQSL |
| | | DRLMNPLIDQYLYYLSRTINGSGQNQQTLKFSVAGPSNMAVQGRNYIPGPSYRQ |
| | | QRVSTTVTQNNNSEFAWPGASSWALNGRNSLMNPGPAMASHKEGEDRFFPLSGS |
| | | LIFGKQGTGRDNVDADKVMITNEEEIKTTNPVATESYGQVATNHQSDGTLAVPF |
| | | KAQAQTGWVQNQGILPGMVWQDRDVYLQGPIWAKIPHTDGNFHPSPLMGGFGMK |
| | | HPPPQILIKNTPVPADPPTAFNKDKLNSFITQYSTGQVSVEIEWELQKENSKRW |
| | | NPEIQYTSNYYKSNNVEFAVNTEGVYSEPRPIGTRYLTRNL |
| AAVrh10 | 14 | MAADGYLPDWLEDNLSEGIREWWDLKPGAPKPKANQQKQDDGRGLVLPGYKYLG |
| (amino | | PFNGLDKGEPVNAADAAALEHDKAYDQQLKAGDNPYLRYNHADAEFQERLQEDT |
| acid) | | SFGGNLGRAVFQAKKRVLEPLGLVEEGAKTAPGKKRPVEPSPQRSPDSSTGIGK |
| | | KGQQPAKKRLNFGQTGDSESVPDPQPIGEPPAGPSGLGSGTMAAGGGAPMADNN |
| | | EGADGVGSSSGNWHCDSTWLGDRVITTSTRTWALPTYNNHLYKQISNGTSGGST |
| | | NDNTYFGYSTPWGYFDFNRFHCHFSPRDWQRLINNNWGFRPKRLNFKLFNIQVK |
| | | EVTQNEGTKTIANNLISTIQVFTDSEYQLPYVLGSAHQGCLPPFPADVFMIPQY |
| | | GYLTLNNGSQAVGRSSFYCLEYFPSQMLRTGNNFEFSYQFEDVPFHSSYAHSQS |
| | | LDRLMNPLIDQYLYYLSRTQSTGGTAGTQQLLFSQAGPNNMSAQAKNWLPGPCY |
| | | RQQRVSTTLSQNNNSNFAWTGATKYHLNGRDSLVNPGVAMATHKDDEERFFPSS |
| | | GVLMFGKQGAGKDNVDYSSVMLTSEEEIKTTNPVATEQYGVVADNLQQQNAAPI |
| | | VGAVNSQGALPGMVWQNRDVYLQGPIWAKIPHTDGNFHPSPLMGGFGLKHPPPQ |
| | | ILIKNTPVPADPPTTFSQAKLASFITQYSTGQVSVEIEWELQKENSKRWNPEIQ |
| | | YTSNYYKSTNVDFAVNTDGTYSEPRPIGTRYLTRNL |
| AAVrh10 | 15 | ATGGCTGCCGATGGTTATCTTCCAGATTGGCTCGAGGACAACCTCTCTGAGGGC |
| (DNA) | | ATTCGCGAGTGGTGGGACTTGAAACCTGGAGCCCCGAAACCCAAAGCCAACCAG |
| | | CAAAAGCAGGACGACGGCCGGGGTCTGGTGCTTCCTGGCTACAAGTACCTCGGA |
| | | CCCTTCAACGGACTCGACAAGGGGGAGCCCGTCAACGCGGCGGACGCAGCGGCC |
| | | CTCGAGCACGACAAGGCCTACGACCAGCAGCTCAAAGCGGGTGACAATCCGTAC |
| | | CTGCGGTATAACCACGCCGACGCCGAGTTTCAGGAGCGTCTGCAAGAAGATACG |
| | | TCTTTTGGGGGCAACCTCGGGCGAGCAGTCTTCCAGGCCAAGAAGCGGGTTCTC |
| | | GAACCTCTCGGTCTGGTTGAGGAAGGCGCTAAGACGGCTCCTGGAAAGAAGAGA |
| | | CCGGTAGAGCCATCACCCCAGCGTTCTCCAGACTCCTCTACGGGCATCGGCAAG |
| | | AAAGGCCAGCAGCCCGCGAAAAAGAGACTCAACTTTGGGCAGACTGGCGACTCA |
| | | GAGTCAGTGCCCGACCCTCAACCAATCGGAGAACCCCCCGCAGGCCCCTCTGGT |
| | | CTGGGATCTGGTACAATGGCTGCAGGCGGTGGCGCTCCAATGGCAGACAATAAC |
| | | GAAGGCGCCGACGGAGTGGGTAGTTCCTCAGGAAATTGGCATTGCGATTCCACA |
| | | TGGCTGGGCGACAGAGTCATCACCACCAGCACCCGAACCTGGGCCCTCCCCACC |
| | | TACAACAACCACCTCTACAAGCAAATCTCCAACGGGACTTCGGGAGGAAGCACC |
| | | AACGACAACACCTACTTCGGCTACAGCACCCCCTGGGGGTATTTTGACTTTAAC |
| | | AGATTCCACTGCCACTTCTCACCACGTGACTGGCAGCGACTCATCAACAACAAC |
| | | TGGGGATTCCGGCCCAAGAGACTCAACTTCAAGCTCTTCAACATCCAGGTCAAG |
| | | GAGGTCACGCAGAATGAAGGCACCAAGACCATCGCCAATAACCTTACCAGCACG |
| | | ATTCAGGTCTTTACGGACTCGGAATACCAGCTCCCGTACGTCCTCGGCTCTGCG |
| | | CACCAGGGCTGCCTGCCTCCGTTCCCGGCGGACGTCTTCATGATTCCTCAGTAC |
| | | GGGTACCTGACTCTGAACAATGGCAGTCAGGCCGTGGGCCGTTCCTCCTTCTAC |
| | | TGCCTGGAGTACTTTCCTTCTCAAATGCTGAGAACGGGCAACAACTTTGAGTTC |
| | | AGCTACCAGTTTGAGGACGTGCCTTTTCACAGCAGCTACGCGCACAGCCAAAGC |
| | | CTGGACCGGCTGATGAACCCCCTCATCGACCAGTACCTGTACTACCTGTCTCGG |
| | | ACTCAGTCCACGGGAGGTACCGCAGGAACTCAGCAGTTGCTATTTTCTCAGGCC |
| | | GGGCCTAATAACATGTCGGCTCAGGCCAAAAACTGGCTACCCGGGCCCTGCTAC |
| | | CGGCAGCAACGCGTCTCCACGACACTGTCGCAAAATAACAACAGCAACTTTGCC |
| | | TGGACCGGTGCCACCAAGTATCATCTGAATGGCAGAGACTCTCTGGTAAATCCC |
| | | GGTGTCGCTATGGCAACCCACAAGGACGACGAAGAGCGATTTTTTCCGTCCAGC |
| | | GGAGTCTTAATGTTTGGGAAACAGGGAGCTGGAAAAGACAACGTGGACTATAGC |
| | | AGCGTTATGCTAACCAGTGAGGAAGAAATTAAAACCACCAACCCAGTGGCCACA |
| | | GAACAGTACGGCGTGGTGGCCGATAACCTGCAACAGCAAAACGCCGCTCCTATT |
| | | GTAGGGGCCGTCAACAGTCAAGGAGCCTTACCTGGCATGGTCTGGCAGAACCGG |
| | | GACGTGTACCTGCAGGGTCCTATCTGGGCCAAGATTCCTCACACGGACGGAAAC |
| | | TTTCATCCCTCGCCGCTGATGGGAGGCTTTGGACTGAAACACCCGCCTCCTCAG |
| | | ATCCTGATTAAGAATACACCTGTTCCCGCGGATCCTCCAACTACCTTCAGTCAA |
| | | GCTAAGCTGGCGTCGTTCATCACGCAGTACAGCACCGGACAGGTCAGCGTGGAA |
| | | ATTGAATGGGAGCTGCAGAAAGAAAACAGCAAACGCTGGAACCCAGAGATTCAA |
| | | TACACTTCCAACTACTACAAATCTACAAATGTGGACTTTGCTGTTAACACAGAT |
| | | GGCACTTATTCTGAGCCTCGCCCCATCGGCACCCGTTACCTCACCCGTAATCTG |
| | | TAA |
| AAV2 | 16 | MAADGYLPDWLEDTLSEGIRQWWKLKPGPPPPKPAERHKDDSRGLVLPGYKYLG |
| (amino | | PFNGLDKGEPVNEADAAALEHDKAYDRQLDSGDNPYLKYNHADAEFQERLKEDT |
| acid) | | SFGGNLGRAVFQAKKRVLEPLGLVEEPVKTAPGKKRPVEHSPVEPDSSSGTGKA |
| | | GQQPARKRLNFGQTGDADSVPDPQPLGQPPAAPSGLGINTMATGSGAPMADNNE |
| | | GADGVGNSSGNWHCDSTWMGDRVITTSTRTWALPTYNNHLYKQISSQSGASNDN |
| | | HYFGYSTPWGYFDFNRFHCHFSPRDWQRLINNNWGFRPKRLNFKLFNIQVKEVT |
| | | QNDGITTIANNLTSTVQVFTDSEYQLPYVLGSAHQGCLPPFPADVFMVPQYGYL |
| | | TLNNGSQAVGRSSFYCLEYFPSQMLRTGNNFTFSYTFEDVPFHSSYAHSQSLDR |
| | | LMNPLIDQYLYYLSRINTPSGITTQSRLQFSQAGASDIRDQSRNWLPGPCYRQQ |
| | | RVSKTSADNNNSEYSWTGATKYHLNGRDSLVNPGPAMASHKDDEEKFFPQSGVL |
| | | IFGKQGSEKTNVDIEKVMITDEEEIRTTNPVATEQYGSVSTNLQRGNRQAATAD |
| | | VNTQGVLPGMVWQDRDVYLQGPIWAKIPHTDGHFHPSPLMGGFGLKHPPPQILI |
| | | KNTPVPANPSTTFSAAKFASFITQYSTGQVSVEIEWELQKENSKRWNPEIQYTS |
| | | NYNKSVNVDFTVDINGVYSEPRPIGTRYLTRNL |
| AAV2 | 17 | ATGGCTGCCGATGGTTATCTTCCAGATTGGCTCGAGGACACTCTCTCTGAAGGA |
| (DNA) | | ATAAGACAGTGGTGGAAGCTCAAACCTGGCCCACCACCACCAAAGCCCGCAGAG |
| | | CGGCATAAGGACGACAGCAGGGGTCTTGTGCTTCCTGGGTACAAGTACCTCGGA |
| | | CCCTTCAACGGACTCGACAAGGGAGAGCCGGTCAACGAGGCAGACGCCGCGGCC |
| | | CTCGAGCACGACAAAGCCTACGACCGGCAGCTCGACAGCGGAGACAACCCGTAC |
| | | CTCAAGTACAACCACGCCGACGCGGAGTTTCAGGAGCGCCTTAAAGAAGATACG |
| | | TCTTTTGGGGGCAACCTCGGACGAGCAGTCTTCCAGGCGAAAAAGAGGGTTCTT |
| | | GAACCTCTGGGCCTGGTTGAGGAACCTGTTAAGACGGCTCCGGGAAAAAAGAGG |
| | | CCGGTAGAGCACTCTCCTGTGGAGCCAGACTCCTCCTCGGGAACCGGAAAGGCG |
| | | GGCCAGCAGCCTGCAAGAAAAAGATTGAATTTTGGTCAGACTGGAGACGCAGAC |
| | | TCAGTACCTGACCCCCAGCCTCTCGGACAGCCACCAGCAGCCCCCTCTGGTCTG |
| | | GGAACTAATACGATGGCTACAGGCAGTGGCGCACCAATGGCAGACAATAACGAG |
| | | GGCGCCGACGGAGTGGGTAATTCCTCGGGAAATTGGCATTGCGATTCCACATGG |
| | | ATGGGCGACAGAGTCATCACCACCAGCACCCGAACCTGGGCCCTGCCCACCTAC |
| | | AACAACCACCTCTACAAACAAATTTCCAGCCAATCAGGAGCCTCGAACGACAAT |
| | | CACTACTTTGGCTACAGCACCCCTTGGGGGTATTTTGACTTCAACAGATTCCAC |
| | | TGCCACTTTTCACCACGTGACTGGCAAAGACTCATCAACAACAACTGGGGATTC |
| | | CGACCCAAGAGACTCAACTTCAAGCTCTTTAACATTCAAGTCAAAGAGGTCACG |
| | | CAGAATGACGGTACGACGACGATTGCCAATAACCTTACCAGCACGGTTCAGGTG |
| | | TTTACTGACTCGGAGTACCAGCTCCCGTACGTCCTCGGCTCGGCGCATCAAGGA |
| | | TGCCTCCCGCCGTTCCCAGCAGACGTCTTCATGGTGCCACAGTATGGATACCTC |
| | | ACCCTGAACAACGGGAGTCAGGCAGTAGGACGCTCTTCATTTTACTGCCTGGAG |
| | | TACTTTCCTTCTCAGATGCTGCGTACCGGAAACAACTTTACCTTCAGCTACACT |
| | | TTTGAGGACGTTCCTTTCCACAGCAGCTACGCTCACAGCCAGAGTCTGGACCGT |
| | | CTCATGAATCCTCTCATCGACCAGTACCTGTATTACTTGAGCAGAACAAACACT |
| | | CCAAGTGGAACCACCACGCAGTCAAGGCTTCAGTTTTCTCAGGCCGGAGCGAGT |
| | | GACATTCGGGACCAGTCTAGGAACTGGCTTCCTGGACCCTGTTACCGCCAGCAG |
| | | CGAGTATCAAAGACATCTGCGGATAACAACAACAGTGAATACTCGTGGACTGGA |
| | | GCTACCAAGTACCACCTCAATGGCAGAGACTCTCTGGTGAATCCGGGCCCGGCC |
| | | ATGGCAAGCCACAAGGACGATGAAGAAAAGTTTTTTCCTCAGAGCGGGGTTCTC |
| | | ATCTTTGGGAAGCAAGGCTCAGAGAAAACAAATGTGGACATTGAAAAGGTCATG |
| | | ATTACAGACGAAGAGGAAATCAGGACAACCAATCCCGTGGCTACGGAGCAGTAT |
| | | GGTTCTGTATCTACCAACCTCCAGAGAGGCAACAGACAAGCAGCTACCGCAGAT |
| | | GTCAACACACAAGGCGTTCTTCCAGGCATGGTCTGGCAGGACAGAGATGTGTAC |
| | | CTTCAGGGGCCCATCTGGGCAAAGATTCCACACACGGACGGACATTTTCACCCC |
| | | TCTCCCCTCATGGGTGGATTCGGACTTAAACACCCTCCTCCACAGATTCTCATC |
| | | AAGAACACCCCGGTACCTGCGAATCCTTCGACCACCTTCAGTGCGGCAAAGTTT |
| | | GCTTCCTTCATCACACAGTACTCCACGGGACAGGTCAGCGTGGAGATCGAGTGG |
| | | GAGCTGCAGAAGGAAAACAGCAAACGCTGGAATCCCGAAATTCAGTACACTTCC |
| | | AACTACAACAAGTCTGTTAATGTGGACTTTACTGTGGACACTAATGGCGTGTAT |
| | | TCAGAGCCTCGCCCCATTGGCACCAGATACCTGACTCGTAATCTGTAA |
| AAV1 | 18 | MAADGYLPDWLEDNLSEGIREWWDLKPGAPKPKANQQKQDDGRGLVLPGYKYLG |
| (amino | | PFNGLDKGEPVNAADAAALEHDKAYDQQLKAGDNPYLRYNHADAEFQERLQEDT |
| acid) | | SFGGNLGRAVFQAKKRVLEPLGLVEEGAKTAPGKKRPVEQSPQEPDSSSGIGKT |
| | | GQQPAKKRLNFGQTGDSESVPDPQPLGEPPATPAAVGPTTMASGGGAPMADNNE |
| | | GADGVGNASGNWHCDSTWLGDRVITTSTRIWALPTYNNHLYKQISSASTGASND |
| | | NHYFGYSTPWGYFDFNRFHCHFSPRDWQRLINNNWGFRPKRLNFKLFNIQVKEV |
| | | TTNDGVTTIANNLTSTVQVFSDSEYQLPYVLGSAHQGCLPPFPADVEMIPQYGY |
| | | LTLNNGSQAVGRSSFYCLEYFPSQMLRTGNNFTFSYTFEEVPFHSSYAHSQSLD |
| | | RLMNPLIDQYLYYLNRTQNQSGSAQNKDLLFSRGSPAGMSVQPKNWLPGPCYRQ |
| | | QRVSKTKTDNNNSNFTWTGASKYNLNGRESIINPGTAMASHKDDEDKFFPMSGV |
| | | MIFGKESAGASNTALDNVMITDEEEIKATNPVATERFGTVAVNFQSSSTDPATG |
| | | DVHAMGALPGMVWQDRDVYLQGPIWAKIPHTDGHFHPSPLMGGFGLKNPPPQIL |
| | | IKNTPVPANPPAEFSATKFASFITQYSTGQVSVEIEWELQKENSKRWNPEVQYT |
| | | SNYAKSANVDFTVDNNGLYTEPRPIGTRYLTRPL |
| AAV1 | 19 | atggctgccgatggttatcttccagattggctcgaggacaacctctctgagggc |
| (DNA) | | attcgcgagtggtgggacttgaaacctggagccccgaagcccaaagccaaccag |
| | | caaaagcaggacgacggccggggtctggtgcttcctggctacaagtacctcgga |
| | | cccttcaacggactcgacaagggggagcccgtcaacgcggcggacgcagcggcc |
| | | ctcgagcacgacaaggcctacgaccagcagctcaaagcgggtgacaatccgtac |
| | | ctgcggtataaccacgccgacgccgagtttcaggagcgtctgcaagaagatacg |
| | | tcttttgggggcaacctcgggcgagcagtcttccaggccaagaagcgggttctc |
| | | gaacctctcggtctggttgaggaaggcgctaagacggctcctggaaagaaacgt |
| | | ccggtagagcagtcgccacaagagccagactcctcctcgggcatcggcaagaca |
| | | ggccagcagcccgctaaaaagagactcaattttggtcagactggcgactcagag |
| | | tcagtccccgatccacaacctctcggagaacctccagcaacccccgctgctgtg |
| | | ggacctactacaatggcttcaggcggtggcgcaccaatggcagacaataacgaa |
| | | ggcgccgacggagtgggtaatgcctcaggaaattggcattgcgattccacatgg |
| | | ctgggcgacagagtcatcaccaccagcacccgcacctgggccttgcccacctac |
| | | aataaccacctctacaagcaaatctccagtgcttcaacgggggccagcaacgac |
| | | aaccactacttcggctacagcaccccctgggggtattttgatttcaacagattc |
| | | cactgccacttttcaccacgtgactggcagcgactcatcaacaacaattgggga |
| | | ttccggcccaagagactcaacttcaaactcttcaacatccaagtcaaggaggtc |
| | | acgacgaatgatggcgtcacaaccatcgctaataaccttaccagcacggttcaa |
| | | gtcttctcggactcggagtaccagcttccgtacgtcctcggctctgcgcaccag |
| | | ggctgcctccctccgttcccggcggacgtgttcatgattccgcaatacggctac |
| | | ctgacgctcaacaatggcagccaagccgtgggacgttcatccttttactgcctg |
| | | gaatatttcccttctcagatgctgagaacgggcaacaactttaccttcagctac |
| | | acctttgaggaagtgcctttccacagcagctacgcgcacagccagagcctggac |
| | | cggctgatgaatcctctcatcgaccaatacctgtattacctgaacagaactcaa |
| | | aatcagtccggaagtgcccaaaacaaggacttgctgtttagccgtgggtctcca |
| | | gctggcatgtctgttcagcccaaaaactggctacctggaccctgttatcggcag |
| | | cagcgcgtttctaaaacaaaaacagacaacaacaacagcaattttacctggact |
| | | ggtgcttcaaaatataacctcaatgggcgtgaatccatcatcaaccctggcact |
| | | gctatggcctcacacaaagacgacgaagacaagttctttcccatgagcggtgtc |
| | | atgatttttggaaaagagagcgccggagcttcaaacactgcattggacaatgtc |
| | | atgattacagacgaagaggaaattaaagccactaaccctgtggccaccgaaaga |
| | | tttgggaccgtggcagtcaatttccagagcagcagcacagaccctgcgaccgga |
| | | gatgtgcatgctatgggagcattacctggcatggtgtggcaagatagagacgtg |
| | | tacctgcagggtcccatttgggccaaaattcctcacacagatggacactttcac |
| | | ccgtctcctcttatgggcggctttggactcaagaacccgcctcctcagatcctc |
| | | atcaaaaacacgcctgttcctgcgaatcctccggcggagttttcagctacaaag |
| | | tttgcttcattcatcacccaatactccacaggacaagtgagtgtggaaattgaa |
| | | tgggagctgcagaaagaaaacagcaagcgctggaatcccgaagtgcagtacaca |
| | | tccaattatgcaaaatctgccaacgttgattttactgtggacaacaatggactt |
| | | tatactgagcctcgccccattggcacccgttaccttacccgtcccctgtaa |
| AAV5 | 20 | MSFVDHPPDWLEEVGEGLREFLGLEAGPPKPKPNQQHQDQARGLVLPGYNYLGP |
| (amino | | GNGLDRGEPVNRADEVAREHDISYNEQLEAGDNPYLKYNHADAEFQEKLADDTS |
| acid) | | FGGNLGKAVFQAKKRVLEPFGLVEEGAKTAPTGKRIDDHFPKRKKARTEEDSKP |
| | | STSSDAEAGPSGSQQLQIPAQPASSLGADTMSAGGGGPLGDNNQGADGVGNASG |
| | | DWHCDSTWMGDRVVTKSTRTWVLPSYNNHQYREIKSGSVDGSNANAYFGYSTPW |
| | | GYFDFNRFHSHWSPRDWQRLINNYWGFRPRSLRVKIFNIQVKEVTVQDSTTTIA |
| | | NNLTSTVQVFTDDDYQLPYVVGNGTEGCLPAFPPQVFTLPQYGYATLNRDNTEN |
| | | PTERSSFFCLEYFPSKMLRTGNNFEFTYNFEEVPFHSSFAPSQNLFKLANPLVD |
| | | QYLYRFVSTNNTGGVQFNKNLAGRYANTYKNWFPGPMGRTQGWNLGSGVNRASV |
| | | SAFAITNRMELEGASYQVPPQPNGMINNLQGSNTYALENTMIFNSQPANPGTTA |
| | | TYLEGNMLITSESETQPVNRVAYNVGGQMATNNQSSTTAPATGTYNLQEIVPGS |
| | | VWMERDVYLQGPIWAKIPETGAHFHPSPAMGGFGLKHPPPMMLIKNTPVPGNIT |
| | | SFSDVPVSSFITQYSTGQVTVEMEWELKKENSKRWNPEIQYTNNYNDPQFVDFA |
| | | PDSTGEYRTTRPIGTRYLTRPL |
| AAV5 | 21 | atgtcttttgttgatcaccctccagattggttggaagaagttggtgaaggtctt |
| (DNA) | | cgcgagtttttgggccttgaagcgggcccaccgaaaccaaaacccaatcagcag |
| | | catcaagatcaagcccgtggtcttgtgctgcctggttataactatctcggaccc |
| | | ggaaacggtctcgatcgaggagagcctgtcaacagggcagacgaggtcgcgcga |
| | | gagcacgacatctcgtacaacgagcagcttgaggcgggagacaacccctacctc |
| | | aagtacaaccacgcggacgccgagtttcaggagaagctcgccgacgacacatcc |
| | | ttcgggggaaacctcggaaaggcagtctttcaggccaagaaaagggttctcgaa |
| | | ccttttggcctggttgaagagggtgctaagacggcccctaccggaaagcggata |
| | | gacgaccactttccaaaaagaaagaaggcccggaccgaagaggactccaagcct |
| | | tccacctcgtcagacgccgaagctggacccagcggatcccagcagctgcaaatc |
| | | ccagcccaaccagcctcaagtttgggagctgatacaatgtctgcgggaggtggc |
| | | ggcccattgggcgacaataaccaaggtgccgatggagtgggcaatgcctcggga |
| | | gattggcattgcgattccacgtggatgggggacagagtcgtcaccaagtccacc |
| | | cgaacctgggtgctgcccagctacaacaaccaccagtaccgagagatcaaaagc |
| | | ggctccgtcgacggaagcaacgccaacgcctactttggatacagcaccccctgg |
| | | gggtactttgactttaaccgcttccacagccactggagcccccgagactggcaa |
| | | agactcatcaacaactactggggcttcagaccccggtccctcagagtcaaaatc |
| | | ttcaacattcaagtcaaagaggtcacggtgcaggactccaccaccaccatcgcc |
| | | aacaacctcacctccaccgtccaagtgtttacggacgacgactaccagctgccc |
| | | tacgtcgtcggcaacgggaccgagggatgcctgccggccttccctccgcaggtc |
| | | tttacgctgccgcagtacggttacgcgacgctgaaccgcgacaacacagaaaat |
| | | cccaccgagaggagcagcttcttctgcctagagtactttcccagcaagatgctg |
| | | agaacgggcaacaactttgagtttacctacaactttgaggaggtgcccttccac |
| | | tccagcttcgctcccagtcagaacctcttcaagctggccaacccgctggtggac |
| | | cagtacttgtaccgcttcgtgagcacaaataacactggcggagtccagttcaac |
| | | aagaacctggccgggagatacgccaacacctacaaaaactggttcccggggccc |
| | | atgggccgaacccagggctggaacctgggctccggggtcaaccgcgccagtgtc |
| | | agcgccttcgccacgaccaataggatggagctcgagggcgcgagttaccaggtg |
| | | cccccgcagccgaacggcatgaccaacaacctccagggcagcaacacctatgcc |
| | | ctggagaacactatgatcttcaacagccagccggcgaacccgggcaccaccgcc |
| | | acgtacctcgagggcaacatgctcatcaccagcgagagcgagacgcagccggtg |
| | | aaccgcgtggcgtacaacgtcggcgggcagatggccaccaacaaccagagctcc |
| | | accactgcccccgcgaccggcacgtacaacctccaggaaatcgtgcccggcagc |
| | | gtgtggatggagagggacgtgtacctccaaggacccatctgggccaagatccca |
| | | gagacgggggcgcactttcacccctctccggccatgggcggattcggactcaaa |
| | | cacccaccgcccatgatgctcatcaagaacacgcctgtgcccggaaatatcacc |
| | | agcttctcggacgtgcccgtcagcagcttcatcacccagtacagcaccgggcag |
| | | gtcaccgtggagatggagtgggagctcaagaaggaaaactccaagaggtggaac |
| | | ccagagatccagtacacaaacaactacaacgacccccagtttgtggactttgcc |
| | | ccggacagcaccggggaatacagaaccaccagacctatcggaacccgatacctt |
| | | acccgacccctttaa |

In some embodiments, the AAV capsid polypeptide, e.g., the AAV capsid variant, comprises the amino acid sequence of any of SEQ ID NOs: 1, 11, 138, 12, 13, 14, 16, 18, 20, or an amino acid sequence substantially identical (e.g., having at least 70%, 75%, 80%, 85%, 90%, 92%, 95%, 97%, 98%, or 99% sequence identity) thereto. In some embodiments the AAV capsid polypeptide, e.g., the AAV capsid variant, comprises an amino acid sequence comprising at least one, two, or three modifications, e.g., substitutions (e.g., conservative substitutions), insertions, or deletions, but no more than 30, 20, or 10 modifications, e.g., substitutions (e.g., conservative substitutions), insertions, or deletions, relative to the amino acid sequence of any of SEQ ID NOs: 1, 11, 138, 12, 13, 14, 16, 18, 20. In some embodiments, the AAV capsid polypeptide, e.g., the AAV capsid variant, comprises an amino acid sequence encoded by the nucleotide sequence of any of SEQ ID NOs: 137, 15, 17, 19, 21, or a nucleotide sequence substantially identical (e.g., having at least 70%, 75%, 80%, 85%, 90%, 92%, 95%, 97%, 98%, or 99% sequence identity) thereto.

In some embodiments, the nucleotide sequence encoding the AAV capsid polypeptide, e.g., the AAV capsid variant, comprises the nucleotide sequence of any of SEQ ID NOs: 137, 15, 17, 19, or 21, or a nucleotide sequence substantially identical (e.g., having at least 70%, 75%, 80%, 85%, 90%, 92%, 95%, 97%, 98%, or 99% sequence identity) thereto. In some embodiments, the nucleotide sequence encoding the AAV capsid polypeptide, e.g., the AAV capsid variant, comprises a nucleotide sequence comprising at least one, two, or three modifications, e.g., substitutions (e.g., conservative substitutions), insertions, or deletions, but no more than 30, 20, or 10 modifications, e.g., substitutions (e.g., conservative substitutions), insertions, or deletions, relative to the nucleotide sequence of any of SEQ ID NOs: 137, 15, 17, 19, or 21.

In some embodiments, the AAV capsid polypeptide, e.g., the AAV capsid variant, comprises the amino acid sequence of SEQ ID NO: 138 or an amino acid sequence substantially identical (e.g., having at least 70%, 75%, 80%, 85%, 90%, 92%, 95%, 97%, 98%, or 99% sequence identity) thereto. In some embodiments the AAV capsid polypeptide, e.g., the AAV capsid variant, comprises an amino acid sequence comprising at least one, two, or three modifications, e.g., substitutions (e.g., conservative substitutions), but no more than 30, 20, or 10 modifications, e.g., substitutions (e.g., conservative substitutions), relative to the amino acid sequence of SEQ ID NO: 138. In some embodiments, the AAV capsid polypeptide, e.g., the AAV capsid variant, comprises an amino acid sequence encoded by the nucleotide sequence of SEQ ID NO: 137 or a nucleotide sequence substantially identical (e.g., having at least 70%, 75%, 80%, 85%, 90%, 92%, 95%, 97%, 98%, or 99% sequence identity) thereto. In some embodiments, the nucleotide sequence encoding the AAV capsid polypeptide, e.g., the AAV capsid variant, comprises the nucleotide sequence of SEQ ID NO: 137 or a nucleotide sequence substantially identical (e.g., having at least 70%, 75%, 80%, 85%, 90%, 92%, 95%, 97%, 98%, or 99% sequence identity) thereto. In some embodiments, the AAV capsid polypeptide, e.g., the AAV capsid variant, comprises substitution at position K449, e.g., a K449R substitution, numbered according to SEQ ID NO: 138.

In some embodiments, the AAV capsid polypeptide, e.g., the AAV capsid variant, comprises a peptide comprising the amino acid sequence of TLAVPFK (SEQ ID NO: 1262). In some embodiments, the peptide is present immediately subsequent to position 588, relative to a reference sequence numbered according to SEQ ID NO: 138. In some embodiments, the capsid polypeptide comprises the amino acid substitutions of A587D and Q588G, numbered according to SEQ ID NO: 138.

In some embodiments, the AAV capsid polypeptide, e.g., the AAV capsid variant, comprises the amino acid substitution of K449R, numbered according to SEQ ID NO: 138; and a peptide comprising the amino acid sequence of TLAVPFK, wherein the peptide is present immediately subsequent to position 588, relative to a reference sequence numbered according to SEQ ID NO: 138.

In some embodiments, the AAV capsid polypeptide, e.g., the AAV capsid variant, comprises the amino acid substitution of K449R, numbered according to SEQ ID NO: 138; an peptide comprising the amino acid sequence of TLAVPFK (SEQ ID NO: 1262), wherein the insert is present immediately subsequent to position 588, relative to a reference sequence numbered according to SEQ ID NO: 138; and the amino acid substitutions of A587D and Q588G, numbered according to SEQ ID NO: 138.

In some embodiments, the AAV capsid polypeptide, e.g., the AAV capsid variant, comprises a peptide comprising the amino acid sequence of TLAVPFK (SEQ ID NO: 1262), wherein the insert is present immediately subsequent to position 588, relative to a reference sequence numbered according to SEQ ID NO: 138; and the amino acid substitutions of A587D and Q588G, numbered according to SEQ ID NO: 138.

In some embodiments, the AAV capsid polypeptide, e.g., the AAV capsid variant, comprises the amino acid sequence of SEQ ID NO: 11 or an amino acid sequence substantially identical (e.g., having at least 70%, 75%, 80%, 85%, 90%, 92%, 95%, 97%, 98%, or 99% sequence identity) thereto. In some embodiments the AAV capsid polypeptide, e.g., the AAV capsid variant, comprises an amino acid sequence comprising at least one, two, or three modifications, e.g., substitutions (e.g., conservative substitutions), but no more than 30, 20, or 10 modifications, e.g., substitutions (conservative substitutions), relative to the amino acid sequence of SEQ ID NO: 11, optionally wherein position 449 is not R.

In some embodiments, the capsid polypeptide, comprises the amino acid sequence of SEQ ID NO: 1 or an amino acid sequence substantially identical (e.g., having at least 70%, 75%, 80%, 85%, 90%, 92%, 95%, 97%, 98%, or 99% sequence identity) thereto. In some embodiments the AAV capsid polypeptide, e.g., the AAV capsid variant, comprises an amino acid sequence comprising at least one, two, or three modifications, e.g., substitutions (e.g., conservative substitutions), but no more than 30, 20, or 10 modifications, e.g., substitutions (e.g., conservative substitutions), relative to the amino acid sequence of SEQ ID NO: 1.

In some embodiments, an AAV capsid variant disclosed herein comprises a modification in loop VIII of AAV9, e.g., at positions between 580-599, e.g., at positions 587, 588, 589, and/or 590, numbered relative to SEQ ID NO: 5, 8, 138 or 3636-3647. In some embodiments, loop (e.g., loop VIII) is used interchangeably herein with the term variable region (e.g., variable region VIII), or VR (e.g., VR-VIII). In some embodiments loop VIII comprises positions 580-599 (e.g., amino acids VATNHQSAQAQAQTGWVQNQ (SEQ ID NO: 1195)), numbered according to SEQ ID NO: 138. In some embodiments, loop VIII comprises positions 582-593 (e.g., amino acids TNHQSAQAQAQT (SEQ ID NO: 1196)), numbered according to SEQ ID NO: 138. In some embodiments loop VIII comprises positions 587-593 (e.g., amino acids AQAQAQT (SEQ ID NO: 1197)), numbered according to SEQ ID NO: 138. In some embodiments loop VIII comprises positions 587-590 (e.g., amino acids AQAQ), numbered according to SEQ ID NO: 138. In some embodiments, loop VIII or variable region VIII (VR-VIII) is as described in DiMattia et al. “Structural Insights into the Unique Properties of the Adeno-Associated Virus Serotype 9,” Journal of Virology, 12(86):6947-6958 (the contents of which are hereby incorporated by reference in their entirety), e.g., comprising positions 581-593 (e.g., ATNHQSAQAQAQT (SEQ ID NO: 1198)), numbered according to SEQ ID NO: 138.

In some embodiments, an AAV particle described herein comprises an AAV capsid polypeptide, e.g., an AAV capsid variant. In some embodiments, the AAV capsid polypeptide, e.g., the AAV capsid variant, comprises a peptide sequence as described in Table 2, e.g., any one of peptides 1, 2, 3, 4, 5, 6, 7, 8, 9, 10, 11, or 12. In some embodiments, the peptide may increase distribution of an AAV particle to a cell, region, or tissue of the CNS. The cell of the CNS may be, but is not limited to, neurons (e.g., excitatory, inhibitory, motor, sensory, autonomic, sympathetic, parasympathetic, Purkinje, Betz, etc.), glial cells (e.g., microglia, astrocytes, oligodendrocytes) and/or supporting cells of the brain such as immune cells (e.g., T cells). The tissue of the CNS may be, but is not limited to, the cortex (e.g., frontal, parietal, occipital, temporal), thalamus, hypothalamus, striatum, putamen, caudate nucleus, hippocampus, entorhinal cortex, basal ganglia, or deep cerebellar nuclei. In some embodiments, the peptide may increase biodistribution of an AAV particle to a cell, region, or tissue of the PNS. The cell or tissue of the PNS may be, but is not limited to, a dorsal root ganglion (DRG). In some embodiments, the peptide may increase biodistribution of an AAV particle to the CNS (e.g., the cortex) after intravenous administration. In some embodiments, the peptide may increase biodistribution of an AAV particle to the CNS (e.g., the cortex) following focused ultrasound (FUS), e.g., coupled with the intravenous administration of microbubbles (FUS-MB), or MRI-guided FUS coupled with intravenous administration. In some embodiments, the peptide may increase biodistribution of an AAV particle to the PNS (e.g., DRG) after intravenous administration. In some embodiments, the peptide may increase biodistribution of an AAV particle to the PNS (e.g., DRG) following focused ultrasound (FUS), e.g., coupled with the intravenous administration of microbubbles (FUS-MB), or MRI-guided FUS coupled with intravenous administration. In some embodiments, the peptide may increase biodistribution of an AAV particle to a cell, region, or tissue of a muscle. In some embodiments, the muscle is a heart muscle. In some embodiments, the peptide may direct an AAV particle to a muscle cell, region, or tissue after intravenous administration.

TABLE 2
Exemplary Peptide Sequences

| | SEQ | Amino | SEQ | |
|---|---|---|---|---|
| Pep- | ID | Acid | ID | Nucleotide |
| tide | NO: | Sequence | NO: | Sequence |
| 1 | 3648 | PLNGAVH | 3660 | CCGCTTAATGGTGCCGTCCAT |
| | | LY | | CTTTAT |
| 2 | 3649 | RDSPKGW | 3661 | CGTGATTCTCCGAAGGGTTGG |
| | | | | CA |
| 3 | 3650 | YSTDVRM | 3662 | TATTCTACGGATGTGAGGATG |
| | | | | CA |
| 4 | 3651 | IVMNSLK | 3663 | ATTGTTATGAATTCGTTGAAG |
| | | | | GC |
| 5 | 3652 | RESPRGL | 3664 | CGGGAGAGTCCTCGTGGGCTG |
| | | | | CA |
| 6 | 3653 | SENDTRA | 3665 | AGTTTTAATGATACTAGGGCT |
| | | | | CA |
| 7 | 3654 | GGTLAVV | 3666 | GGTGGTACGTTGGCCGTCGTG |
| | | SL | | TCGCTT |
| 8 | 3655 | YGLPKGP | 3667 | TATGGGTTGCCGAAGGGTCCT |
| 9 | 3656 | STGTLRL | 3668 | TCGACTGGGACGCTTCGGCTT |
| 10 | 3657 | YSTDERM | 3669 | TATTCGACGGATGAGAGGATG |
| 11 | 3658 | YSTDERK | 3670 | TATTCGACGGATGAGAGGAAG |
| 12 | 3659 | YVSSVKM | 3671 | TATGTTTCGTCTGTTAAGATG |
| 13 | 314 | PLNGAVH | 6 | CCGCTTAATGGTGCCGTCCAT |
| | | LYAQAQT | | CTTTATGCTCAGGCGCAGACC |
| | | GWVPN | | GGCTGGGTTCCGAAC |
| 14 | 566 | PLNGAVH | 9 | CCGCTGAATGGTGCGGTGCAT |
| | | LYAQAQL | | CTGTATGCGCAGGCGCAGCTG |
| | | SPVKN | | TCTCCGGTGAAGAAT |

In some embodiments, the AAV capsid polypeptide, e.g., the AAV capsid variant, comprises at least 3, 4, 5, 6, 7, 8, or 9 consecutive amino acids from the amino acid sequence of any of SEQ ID NO: 3648-3659. In some embodiments, the amino acid sequence is present in loop VIII. In some embodiments, the amino acid sequence is present immediately subsequent to position 586, 588, or 589, relative to a reference sequence numbered according to the amino acid sequence of SEQ ID NO: 138.

In some embodiments, the AAV capsid polypeptide, e.g., the AAV capsid variant, comprises an amino acid sequence comprising at least one, two, or three but no more than four modifications, e.g., substitutions, (e.g., conservative substitutions), insertions, or deletions, relative to any of the amino acid sequence of Table 20. In some embodiments, the AAV capsid polypeptide, e.g., the AAV capsid variant, comprises an amino acid sequence comprising at least one, two, or three but no more than four different amino acids, relative to any of the amino acid sequence of Table 20. In some embodiments, the AAV capsid variant comprises an amino acid sequence comprising at least one, two, or three but no more than four modifications, e.g., substitutions, (e.g., conservative substitutions), insertions, or deletions, relative to the amino acid sequence of any of SEQ ID NO: 3648-3659. In some embodiments, the AAV capsid variant comprises an amino acid sequence comprising at least one, two, or three but no more than four different amino acids relative to the amino acid sequence of any of SEQ ID NO: 3648-3659. In some embodiments, the amino acid sequence is present in loop VIII. In some embodiments, the amino acid sequence is present immediately subsequent to position 586, 588, or 589, relative to a reference sequence numbered according to the amino acid sequence of SEQ ID NO: 138.

In some embodiments, the AAV capsid polypeptide, e.g., the AAV capsid variant, comprises at least 3, 4, 5, 6, 7, 8, or 9 consecutive amino acids from the amino acid sequence of any of SEQ ID NO: 3648-3659. In some embodiments, the 3 consecutive amino acids comprise PLN. In some embodiments, the 4 consecutive amino acids comprise PLNG (SEQ ID NO: 3678). In some embodiments, the 5 consecutive amino acids comprise PLNGA (SEQ ID NO: 3679). In some embodiments, the 6 consecutive amino acids comprise PLNGAV (SEQ ID NO: 3680). In some embodiments, the 7 consecutive amino acids comprise PLNGAVH (SEQ ID NO: 3681). In some embodiments, the 8 consecutive amino acids comprise PLNGAVHL (SEQ ID NO: 3682). In some embodiments, the 9 consecutive amino acids comprise PLNGAVHLY (SEQ ID NO: 3648).

In some embodiments, the four consecutive amino acids comprise NGAV (SEQ ID NO: 3683). In some embodiments, the four consecutive amino acids comprise GAVH (SEQ ID NO: 3684). In some embodiments, the five consecutive amino acids comprise NGAVH (SEQ ID NO: 3685). In some embodiments, the five consecutive amino acids comprise GAVHL (SEQ ID NO: 3686). In some embodiments, the five consecutive amino acids comprise AVHLY (SEQ ID NO: 3687). In some embodiments, the six consecutive amino acids comprise NGAVHL (SEQ ID NO: 3688). In some embodiments, the seven consecutive amino acids comprise NGAVHLY (SEQ ID NO: 3689).

In some embodiments, the 3 consecutive amino acids comprise YST. In some embodiments, the 4 consecutive amino acids comprise YSTD (SEQ ID NO: 3690). In some embodiments, the 5 consecutive amino acids comprise YSTDE (SEQ ID NO: 3691). In some embodiments, the 5 consecutive amino acids comprise YSTDV (SEQ ID NO: 3700). In some embodiments, the 6 consecutive amino acids comprise YSTDER (SEQ ID NO: 3692). In some embodiments, the 6 consecutive amino acids comprise YSTDVR (SEQ ID NO: 3701). In some embodiments, the 7 consecutive amino acids comprise YSTDERM (SEQ ID NO: 3657). In some embodiments, the 7 consecutive amino acids comprise YSTDERK (SEQ ID NO: 3658). In some embodiments, the 7 consecutive amino acids comprise YSTDVRM (SEQ ID NO: 3650).

In some embodiments, the 3 consecutive amino acids comprise IVM. In some embodiments, the 4 consecutive amino acids comprise IVMN (SEQ ID NO: 3693). In some embodiments, the 5 consecutive amino acids comprise IVMNS (SEQ ID NO: 3694). In some embodiments, the 6 consecutive amino acids comprise IVMNSL (SEQ ID NO: 3695). In some embodiments, the 7 consecutive amino acids comprise IVMNSLK (SEQ ID NO: 3651).

In some embodiments, the AAV capsid polypeptide, e.g., the AAV capsid variant, comprises an amino acid sequence comprising at least one, two, or three but no more than four modifications, e.g., substitutions (e.g., conservative substitutions), relative to the amino acid sequence of any of SEQ ID NO: 3648-3659. In some embodiments, the modification is a conservative substitution.

In some embodiments, the AAV capsid polypeptide, e.g., the AAV capsid variant, comprises the amino acid sequence of PLNGAVHLY (SEQ ID NO: 3648), an amino acid sequence comprising at least one, two, or three but no more than four modifications, e.g., substitutions (e.g., conservative substitutions), insertions, or deletions, relative to the amino acid sequence of PLNGAVHLY (SEQ ID NO: 3648), or an amino acid sequence comprising at least one, two, or three but no more than four different amino acids relative to the amino acid sequence of PLNGAVHLY (SEQ ID NO: 3648), optionally wherein position 7 is H.

In some embodiments, the AAV capsid polypeptide, e.g., the AAV capsid variant, comprises the amino acid sequence of RDSPKGW (SEQ ID NO: 3649), an amino acid sequence comprising at least one, two, or three modifications, e.g., substitutions (e.g., conservative substitutions), insertions, or deletions, but no more than four modifications, e.g., substitutions (e.g., conservative substitutions), insertions, or deletions, relative to the amino acid sequence of RDSPKGW (SEQ ID NO: 3649), or an amino acid sequence comprising at least one, two, or three but no more than four different amino acids relative to the amino acid sequence of RDSPKGW (SEQ ID NO: 3649).

In some embodiments, the AAV capsid polypeptide, e.g., the AAV capsid variant, comprises the amino acid sequence of IVMNSLK (SEQ ID NO: 3651), an amino acid sequence comprising at least one, two, or three modifications, e.g., substitutions (e.g., conservative substitutions), insertions, or deletions, but no more than four modifications, e.g., substitutions (e.g., conservative substitutions), insertions, or deletions, relative to the amino acid sequence of IVMNSLK (SEQ ID NO: 3651), or an amino acid sequence comprising at least one, two, or three but no more than four different amino acids relative to the amino acid sequence of IVMNSLK (SEQ ID NO: 3651).

In some embodiments, the AAV capsid polypeptide, e.g., the AAV capsid variant, comprises the amino acid sequence of YSTDVRM (SEQ ID NO: 3650), an amino acid sequence comprising at least one, two, or three modifications, e.g., substitutions (e.g., conservative substitutions), insertions, or deletions, but no more than four modifications, e.g., substitutions (e.g., conservative substitutions), insertions, or deletions, relative to the amino acid sequence of YSTDVRM (SEQ ID NO: 3650), or an amino acid sequence comprising at least one, two, or three but no more than four different amino acids relative to the amino acid sequence of YSTDVRM (SEQ ID NO: 3650).

In some embodiments, the AAV capsid polypeptide, e.g., the AAV capsid variant, comprises the amino acid sequence of RESPRGL (SEQ ID NO: 3652), a sequence comprising at least one, two, or three modifications, e.g., substitutions (e.g., conservative substitutions), insertions, or deletions, but no more than four modifications, e.g., substitutions (e.g., conservative substitutions), insertions, or deletions, relative to the amino acid sequence of RESPRGL (SEQ ID NO: 3652), or an amino acid sequence comprising at least one, two, or three but no more than four different amino acids relative to the amino acid sequence of RESPRGL (SEQ ID NO: 3652).

In some embodiments, the AAV capsid variant comprises the amino acid sequence of any of SEQ ID NO: 3648-3659. In some embodiments, the amino acid sequence is present in loop VIII of an AAV capsid variant described herein. In some embodiments, the AAV capsid variant comprises the amino acid sequence of SEQ ID NO: 3648. In some embodiments, the AAV capsid variant comprises the amino acid sequence of SEQ ID NO: 3649. In some embodiments, the AAV capsid variant comprises the amino acid sequence of SEQ ID NO: 3651. In some embodiments, the amino acid sequence is present immediately subsequent to position 586, relative to a reference sequence numbered according to the amino acid sequence of SEQ ID NO: 138. In some embodiments, the amino acid sequence is present immediately subsequent to position 588, relative to a reference sequence numbered according to the amino acid sequence of SEQ ID NO: 138. In some embodiments, the amino acid sequence is present immediately subsequent to position 589, relative to a reference sequence numbered according to the amino acid sequence of SEQ ID NO: 138.

In some embodiments, the AAV capsid polypeptide, e.g., the AAV capsid variant (e.g., an AAV capsid variant described herein), comprises an amino acid sequence encoded by the nucleotide sequence of any one of SEQ ID NOs: 3660-3671, or a nucleotide sequence substantially identical (e.g., having at least 70%, 75%, 80%, 85%, 90%, 92%, 95%, 97%, 98%, or 99% sequence identity) thereto. In some embodiments, the AAV capsid, e.g., an AAV capsid variant described herein, comprises an amino acid sequence encoded by a nucleotide sequence comprising at least one, two, three, four, five, six, or seven modifications, e.g., substitutions (e.g., conservative substitutions), insertions, or deletions, but no more than ten modifications, e.g., substitutions (e.g., conservative substitutions), insertions, or deletions, relative to the nucleotide sequences of any of SEQ ID NOs: 3660-3671. In some embodiments, the AAV capsid polypeptide, e.g., the AAV capsid variant (e.g., an AAV capsid variant described herein), comprises an amino acid sequence encoded by a nucleotide sequence comprising at least one, two, three, four, five, six, or seven, but no more than ten different nucleotides relative to the nucleotide sequence of any of SEQ ID NOs: 3660-3671.

In some embodiments, the AAV capsid polypeptide, e.g., the AAV capsid variant (e.g., an AAV capsid variant described herein), comprises an amino acid sequence encoded by the nucleotide sequence of any one of SEQ ID NOs: 3660-3671, or a nucleotide sequence substantially identical (e.g., having at least 70%, 75%, 80%, 85%, 90%, 92%, 95%, 97%, 98%, or 99% sequence identity) thereto. In some embodiments, the AAV capsid, e.g., an AAV capsid variant described herein, comprises an amino acid sequence encoded by a nucleotide sequence comprising at least one, two, three, four, five, six, or seven modifications, e.g., substitutions (e.g., conservative substitutions), insertions, or deletions, but no more than ten modifications, e.g., substitutions (e.g., conservative substitutions), insertions, or deletions, relative to the nucleotide sequences of any of SEQ ID NOs: 3660-3671. In some embodiments, the AAV capsid polypeptide, e.g., the AAV capsid variant (e.g., an AAV capsid variant described herein), comprises an amino acid sequence encoded by a nucleotide sequence comprising at least one, two, three, four, five, six, or seven, but no more than ten different nucleotides relative to the nucleotide sequence of any of SEQ ID NOs: 3660-3671.

In some embodiments, the nucleotide sequence encoding the AAV capsid polypeptide, e.g., the AAV capsid variant (e.g., an AAV capsid variant described herein), comprises the nucleotide sequence of any one of SEQ ID NOs: 3660-3671, or a nucleotide sequence substantially identical (e.g., having at least 70%, 75%, 80%, 85%, 90%, 92%, 95%, 97%, 98%, or 99% sequence identity) thereto. In some embodiments, nucleic acid sequence encoding the AAV capsid variant, e.g., an AAV capsid variant described herein, comprises a nucleotide sequence comprising at least one, two, three, four, five, six, or seven modifications, e.g., substitutions (e.g., conservative substitutions), insertions, or deletions, but no more than ten modifications, e.g., substitutions (e.g., conservative substitutions), insertions, or deletions, of the nucleotide sequences relative to any of SEQ ID NOs: 3660-3671. In some embodiments, the nucleotide sequence encoding the AAV capsid polypeptide, e.g., the AAV capsid variant (e.g., an AAV capsid variant described herein), comprises a nucleotide sequence comprising at least one, two, three, four, five, six, or seven, but no more than ten different nucleotides relative to the nucleotide sequence of any of SEQ ID NOs: 3660-3671.

In some embodiments, the nucleotide sequence encoding the AAV capsid polypeptide, e.g., the AAV capsid variant (e.g., an AAV capsid variant described herein), comprises the nucleotide sequence of SEQ ID NO: 3660, or a nucleotide sequence substantially identical (e.g., having at least 70%, 75%, 80%, 85%, 90%, 92%, 95%, 97%, 98%, or 99% sequence identity) thereto. In some embodiments, the nucleic acid sequence encoding the AAV capsid variant comprises a nucleotide sequence comprising at least one, two, three, four, five, six, or seven modifications, e.g., substitutions (e.g., conservative substitutions), insertions, or deletions, but no more than ten modifications, e.g., substitutions (e.g., conservative substitutions), insertions, or deletions, relative to the nucleotide sequences of SEQ ID NO: 3660. In some embodiments, the nucleotide sequence encoding the AAV capsid polypeptide, e.g., the AAV capsid variant (e.g., an AAV capsid variant described herein), comprises a nucleotide sequence comprising at least one, two, three, four, five, six, or seven, but no more than ten different nucleotides relative to the nucleotide sequence of SEQ ID NO: 3660.

In some embodiments, the nucleotide sequence encoding the AAV capsid polypeptide, e.g., the AAV capsid variant (e.g., an AAV capsid variant described herein), comprises the nucleotide sequence of SEQ ID NO: 3663, or a nucleotide sequence substantially identical (e.g., having at least 70%, 75%, 80%, 85%, 90%, 92%, 95%, 97%, 98%, or 99% sequence identity) thereto. In some embodiments, the nucleic acid sequence encoding the AAV capsid variant comprises a nucleotide sequence comprising at least one, two, three, four, five, six, or seven modifications, e.g., substitutions (e.g., conservative substitutions), insertions, or deletions, but no more than ten modifications, e.g., substitutions (e.g., conservative substitutions), insertions, or deletions, of the nucleotide sequences relative to SEQ ID NO: 3663. In some embodiments, the nucleotide sequence encoding the AAV capsid polypeptide, e.g., the AAV capsid variant (e.g., an AAV capsid variant described herein), comprises a nucleotide sequence comprising at least one, two, three, four, five, six, or seven, but no more than ten different nucleotides relative to the nucleotide sequence of SEQ ID NO: 3663.

In some embodiments, the AAV capsid polypeptide, e.g., the AAV capsid variant, comprises an amino acid residue other than “A” at position 587 and/or an amino acid residue other than “Q” at position 588, numbered according to SEQ ID NO: 138. In some embodiments, the AAV capsid polypeptide, e.g., the AAV capsid variant, comprises one, two, three, or all of an amino acid other than A at position 589, an amino acid other than Q at position 590, an amino acid other than A at position 591, and/or an amino acid other than Q at position 592, relative to a reference sequence numbered according to the amino acid sequence of SEQ ID NO: 138. In some embodiments, the AAV capsid polypeptide, e.g., the AAV capsid variant, comprises one, two, or all of an amino acid other than T at position 593, an amino acid other than G at position 594, and/or an amino acid other than W at position 595, relative to a reference sequence numbered according to the amino acid sequence of SEQ ID NO: 138. In some embodiments, the AAV capsid polypeptide, e.g., the AAV capsid variant, comprises one, two, or all of an amino acid other than V at position 596, an amino acid other than Q at position 597, and/or an amino acid other than N at position 598, relative to a reference sequence numbered according to the amino acid sequence of SEQ ID NO: 138.

In some embodiments, the AAV capsid polypeptide, e.g., the AAV capsid variant, comprises the amino acid sequence of PLNGAVHLY (SEQ ID NO: 3648) wherein the amino acid sequence of PLNGAVHLY (SEQ ID NO: 3648) is present immediately subsequent to position 586, relative to a reference sequence numbered according to the amino acid sequence of SEQ ID NO: 138.

In some embodiments, the polypeptide, e.g., the AAV capsid variant, comprises the amino acid sequence of GGTLAVVSL (SEQ ID NO: 3654), wherein the amino acid sequence of GGTLAVVSL (SEQ ID NO: 3654) is present immediately subsequent to position 586, relative to a reference sequence numbered according to the amino acid sequence of SEQ ID NO: 138.

In some embodiments, the AAV capsid polypeptide, e.g., the AAV capsid variant, comprises the amino acid sequence of IVMNSLK (SEQ ID NO: 3651), wherein the amino acid sequence of IVMNSLK (SEQ ID NO: 3651) is present immediately subsequent to position 588, relative to a reference sequence numbered according to the amino acid sequence of SEQ ID NO: 138.

In some embodiments, the AAV capsid polypeptide, e.g., the AAV capsid variant, comprises the amino acid sequence of any of SEQ ID NOs: 3649, 3650, 3652, 3653, or 3655-3659, wherein the amino acid sequence of any of the aforesaid sequences is present immediately subsequent to position 589, relative to a reference sequence numbered according to the amino acid sequence of SEQ ID NO: 138.

In some embodiments, the AAV capsid polypeptide, e.g., the AAV capsid variant, further comprises a substitution at position K449, e.g., a K449R substitution, numbered according to SEQ ID NO: 138. In some embodiments, the AAV capsid variant further comprises a modification, e.g., an insertion, substitution, and/or deletion in loop I, II, IV, and/or VI.

In some embodiments, the AAV capsid polypeptide, e.g., the AAV capsid variant, comprises one, two, three, or all of an amino acid other than A at position 589, an amino acid other than Q at position 590, an amino acid other than A at position 591, and/or an amino acid other than Q at position 592, relative to a reference sequence numbered according to the amino acid sequence of SEQ ID NO: 138. In some embodiments, the AAV capsid polypeptide, e.g., the AAV capsid variant, comprises one, two, or all of an amino acid other than T at position 593, an amino acid other than G at position 594, and/or an amino acid other than W at position 595, relative to a reference sequence numbered according to the amino acid sequence of SEQ ID NO: 138. In some embodiments, the AAV capsid polypeptide, e.g., the AAV capsid variant, comprises one, two, or all of an amino acid other than V at position 596, an amino acid other than Q at position 597, and/or an amino acid other than N at position 598, relative to a reference sequence numbered according to the amino acid sequence of SEQ ID NO: 138. In some embodiments, the AAV capsid variant further comprises the amino acid sequence of SEQ ID NO: 138, or an amino acid sequence with at least 80% (e.g., at least about 85, 90, 95, 96, 97, 98, or 99%) sequence identity thereto. In some embodiments, the AAV capsid variant further comprises an amino acid sequence encoded by the nucleotide sequence of SEQ ID NO: 137, or a sequence with at least 80% (e.g., at least about 85, 90, 95, 96, 97, 98, or 99%) sequence identity thereto.

In some embodiments, an AAV capsid polypeptide, e.g., the AAV capsid variant, comprises immediately subsequent to position 586, 588, or 589, numbered relative to SEQ ID NO: 138 or corresponding to equivalent positions in any other AAV serotype (e.g., AAV1, AAV2, AAV3, AAV3b, AAV4, AAV5, AAV6, AAV7, AAV8, AAVrh8, AAVrh10, AAVrh32.33, AAVrh74, SEQ ID NO: 1, SEQ ID NO: 11, PHP.N, PHP.B, or an AAV serotype as provided in Table 6 of WO 2021/230987 (the contents of which are hereby incorporated by reference in their entirety)), at least 3, 4, 5, 6, 7, 8, 9, 10, 11, 12, 13, 14, 15, 16, 17, 18, or 19 consecutive amino acids of any of amino acid sequence provided in Tables 2, 20, 23, or 24. In some embodiments, the at least 3, 4, 5, 6, 7, 8, 9, 10, 11, 12, 13, 14, 15, 16, 17, 18, or 19 consecutive amino acids of any of amino acid sequence provided in Tables 2, 20, 23, or 24 replaces at least one, two, three, four, five, six, seven, eight, nine, ten, elven, or all of positions A587, Q588, A589, Q590, A591, Q592, T593, G594, W595, V596, Q597, and/or N598, numbered according to SEQ ID NO: 138 or corresponding to equivalent positions in any other AAV serotype (e.g., AAV1, AAV2, AAV3, AAV3b, AAV4, AAV5, AAV6, AAV7, AAV8, AAVrh8, AAVrh10, AAVrh32.33, AAVrh74, SEQ ID NO: 1, SEQ ID NO: 11, PHP.N, PHP.B, or an AAV serotype as provided in Table 6 of WO 2021/230987 (the contents of which are hereby incorporated by reference in their entirety). In some embodiments, the at least 3, 4, 5, 6, 7, 8, 9, 10, 11, 12, 13, 14, 15, 16, 17, 18, or 19 consecutive amino acids of any of amino acid sequence provided in Tables 2, 20, 23, or 24 replaces positions A587, Q588, or both positions A587 and Q588, numbered according to SEQ ID NO: 138 or corresponding to equivalent positions in any other AAV serotype (e.g., AAV1, AAV2, AAV3, AAV3b, AAV4, AAV5, AAV6, AAV7, AAV8, AAVrh8, AAVrh10, AAVrh32.33, AAVrh74, SEQ ID NO: 1, SEQ ID NO: 11, PHP.N, PHP.B, or an AAV serotype as provided in Table 6 of WO 2021/230987 (the contents of which are hereby incorporated by reference in their entirety). In some embodiments, the AAV capsid variant comprises an amino acid other than the wild-type, e.g., native, amino acid, at one, two, three, four, five, six, seven, eight, nine, ten, eleven or all of positions A587, Q588, A589, Q590, A591, Q592, T593, G594, W595, V596, Q597, and/or N598, numbered according to SEQ ID NO: 138 or corresponding to equivalent positions in any other AAV serotype (e.g., AAV1, AAV2, AAV3, AAV3b, AAV4, AAV5, AAV6, AAV7, AAV8, AAVrh8, AAVrh10, AAVrh32.33, AAVrh74, SEQ ID NO: 1, SEQ ID NO: 11, PHP.N, PHP.B, or an AAV serotype as provided in Table 6 of WO 2021/230987 (the contents of which are hereby incorporated by reference in their entirety). In some embodiments, the AAV capsid variant comprises an amino acid other than the wild-type, e.g., native, amino acid, at position A587, Q588, or both positions A587 and Q588, numbered according to SEQ ID NO: 138 or corresponding to equivalent positions in any other AAV serotype (e.g., AAV1, AAV2, AAV3, AAV3b, AAV4, AAV5, AAV6, AAV7, AAV8, AAVrh8, AAVrh10, AAVrh32.33, AAVrh74, SEQ ID NO: 1, SEQ ID NO: 11, PHP.N, PHP.B, or an AAV serotype as provided in Table 6 of WO 2021/230987 (the contents of which are hereby incorporated by reference in their entirety)). In some embodiments, the AAV capsid variant comprises a modification, e.g., substitution, at one, two, three, four, five, six, seven, eight, nine, ten eleven or all of positions A587, Q588, A589, Q590, A591, Q592, T593, G594, W595, V596, Q597, and/or N598, numbered according to SEQ ID NO: 138 or corresponding to equivalent positions in any other AAV serotype (e.g., AAV1, AAV2, AAV3, AAV3b, AAV4, AAV5, AAV6, AAV7, AAV8, AAVrh8, AAVrh10, AAVrh32.33, AAVrh74, SEQ ID NO: 1, SEQ ID NO: 11, PHP.N, PHP.B, or an AAV serotype as provided in Table 6 of WO 2021/230987 (the contents of which are hereby incorporated by reference in their entirety). In some embodiments, the AAV capsid variant comprises a modification, e.g., substitution, at position A587, Q588, or both positions A587 and Q588, numbered according to SEQ ID NO: 138 or corresponding to equivalent positions in any other AAV serotype (e.g., AAV1, AAV2, AAV3, AAV3b, AAV4, AAV5, AAV6, AAV7, AAV8, AAVrh8, AAVrh10, AAVrh32.33, AAVrh74, SEQ ID NO: 1, SEQ ID NO: 11, PHP.N, PHP.B, or an AAV serotype as provided in Table 6 of WO 2021/230987 (the contents of which are hereby incorporated by reference in their entirety).

In some embodiments, an AAV capsid polypeptide, e.g., an AAV capsid variant, of the present disclosure comprises an amino acid sequence as described herein, e.g. an amino acid sequence of an AAV capsid variant chosen from TTD-001, TTD-002, TTD-003, TTD-004, TTD-005, TTD-006, TTD-007, TTD-008, TTD-009, TTD-010, TTD-011, or TTD-012, e.g., as described in Tables 3 and 4.

In some embodiments, an AAV capsid polypeptide, e.g. the AAV capsid variant, comprises a VP1, VP2, and/or VP3 protein comprising an amino acid sequence described herein, e.g. an amino acid sequence of an AAV capsid variant chosen from TTD-001, TTD-002, TTD-003, TTD-004, TTD-005, TTD-006, TTD-007, TTD-008, TTD-009, TTD-010, TTD-011, or TTD-012, e.g., as described in Tables 3 and 4.

In some embodiments, an AAV capsid polypeptide, e.g., the AAV capsid variant, comprises an amino acid sequence encoded by a nucleotide sequence as described herein, e.g. a nucleotide sequence of an AAV capsid variant chosen from TTD-001, TTD-002, TTD-003, TTD-004, TTD-005, TTD-006, TTD-007, TTD-008, TTD-009, TTD-010, TTD-011, or TTD-012, e.g., as described in Tables 3 and 5.

In some embodiments, a polynucleotide encoding an AAV capsid polypeptide, e.g., an AAV capsid variant, of the present disclosure comprises a nucleotide sequence described herein, e.g. a nucleotide sequence of an AAV capsid variant chosen from TTD-001, TTD-002, TTD-003, TTD-004, TTD-005, TTD-006, TTD-007, TTD-008, TTD-009, TTD-010, TTD-011, or TTD-012, e.g., as described in Tables 3 and 5.

In some embodiments, insertion of a nucleic acid sequence, targeting nucleic acid sequence, or a peptide into a parent AAV sequence generates the non-limiting exemplary full length capsid sequences, e.g., an AAV capsid polypeptide, e.g., an AAV capsid variant, as described in Tables 3-5.

TABLE 3
Exemplary full length capsid sequences (VP1 with insert)

| | VP1 DNA | VP1 PRT | Peptide PRT | Peptide DNA |
|---|---|---|---|---|
| Serotype | SEQ ID NO: | SEQ ID NO: | SEQ ID NO: | SEQ ID NO: |
| TTD-001 | 3623 | 3636 | 3648 | 3660 |
| TTD-002 | 3624 or 3625 | 3637 | 3649 | 3661 |
| TTD-003 | 3626 | 3638 | 3650 | 3662 |
| TTD-004 | 3627 | 3639 | 3651 | 3663 |
| TTD-005 | 3628 | 3640 | 3652 | 3664 |
| TTD-006 | 3629 | 3641 | 3653 | 3665 |
| TTD-007 | 3630 | 3642 | 3654 | 3666 |
| TTD-008 | 3631 | 3643 | 3655 | 3667 |
| TTD-009 | 3632 | 3644 | 3656 | 3668 |
| TTD-010 | 3633 | 3645 | 3657 | 3669 |
| TTD-011 | 3634 | 3646 | 3658 | 3670 |
| TTD-012 | 3635 | 3647 | 3659 | 3671 |
| TTD-013 | 4 | 5 | 314 | 6 |
| TTD-014 | 7 | 8 | 566 | 9 |

TABLE 4
Exemplary full length capsid amino acid sequences

| | SEQ | |
|---|---|---|
| Name and | ID | |
| Annotation | NO: | Amino Acid Sequence |
| TTD-001 | 3636 | MAADGYLPDWLEDNLSEGIREWWALKPGAPQPKANQQHQDNARGLVLP |
| 9mer peptide | | GYKYLGPGNGLDKGEPVNAADAAALEHDKAYDQQLKAGDNPYLKYNHA |
| underlined, starts | | DAEFQERLKEDISFGGNLGRAVFQAKKRLLEPLGLVEEAAKTAPGKKR |
| at position 587 | | PVEQSPQEPDSSAGIGKSGAQPAKKRLNFGQTGDTESVPDPQPIGEPP |
| (immediately | | AAPSGVGSLTMASGGGAPVADNNEGADGVGSSSGNWHCDSQWLGDRVI |
| subsequent to | | ITSTRTWALPTYNNHLYKQISNSTSGGSSNDNAYFGYSTPWGYFDENR |
| position 586); | | FHCHFSPRDWQRLINNNWGFRPKRLNFKLFNIQVKEVTDNNGVKTIAN |
| 743 aa | | NLTSTVQVFTDSDYQLPYVLGSAHEGCLPPFPADVFMIPQYGYLTLND |
| | | GSQAVGRSSFYCLEYFPSQMLRIGNNFQFSYEFENVPFHSSYAHSQSL |
| | | DRLMNPLIDQYLYYLSKTINGSGQNQQTLKFSVAGPSNMAVQGRNYIP |
| | | GPSYRQQRVSTTVTQNNNSEFAWPGASSWALNGRNSLMNPGPAMASHK |
| | | EGEDRFFPLSGSLIFGKQGTGRDNVDADKVMITNEEEIKTINPVATES |
| | | YGQVATNHQSPLNGAVHLYAQAQTGWVQNQGILPGMVWQDRDVYLQGP |
| | | IWAKIPHIDGNFHPSPLMGGFGMKHPPPQILIKNTPVPADPPTAFNKD |
| | | KLNSFITQYSTGQVSVEIEWELQKENSKRWNPEIQYTSNYYKSNNVEF |
| | | AVNTEGVYSEPRPIGTRYLTRNL |
| TTD-002 | 3637 | MAADGYLPDWLEDNLSEGIREWWALKPGAPQPKANQQHQDNARGLVLP |
| 7mer peptide | | GYKYLGPGNGLDKGEPVNAADAAALEHDKAYDQQLKAGDNPYLKYNHA |
| underlined, starts | | DAEFQERLKEDTSFGGNLGRAVFQAKKRLLEPLGLVEEAAKTAPGKKR |
| at position 590 | | PVEQSPQEPDSSAGIGKSGAQPAKKRLNFGQTGDTESVPDPQPIGEPP |
| (immediately | | AAPSGVGSLTMASGGGAPVADNNEGADGVGSSSGNWHCDSQWLGDRVI |
| subsequent to | | TTSTRTWALPTYNNHLYKQISNSTSGGSSNDNAYFGYSTPWGYFDENR |
| position 589); | | FHCHFSPRDWQRLINNNWGFRPKRLNFKLFNIQVKEVTDNNGVKTIAN |
| 743 aa | | NLTSTVQVFTDSDYQLPYVLGSAHEGCLPPFPADVFMIPQYGYLILND |
| | | GSQAVGRSSFYCLEYFPSQMLRTGNNFQFSYEFENVPFHSSYAHSQSL |
| | | DRLMNPLIDQYLYYLSKTINGSGQNQQTLKFSVAGPSNMAVQGRNYIP |
| | | GPSYRQQRVSTTVTQNNNSEFAWPGASSWALNGRNSLMNPGPAMASHK |
| | | EGEDRFFPLSGSLIFGKQGTGRDNVDADKVMITNEEEIKTTNPVATES |
| | | YGQVATNHQSAQARDSPKGWQAQTGWVQNQGILPGMVWQDRDVYLQGP |
| | | IWAKIPHTDGNFHPSPLMGGFGMKHPPPQILIKNTPVPADPPTAFNKD |
| | | KLNSFITQYSTGQVSVEIEWELQKENSKRWNPEIQYTSNYYKSNNVEF |
| | | AVNTEGVYSEPRPIGTRYLTRNL |
| TTD-003 | 3638 | MAADGYLPDWLEDNLSEGIREWWALKPGAPQPKANQQHQDNARGLVLP |
| 7mer peptide | | GYKYLGPGNGLDKGEPVNAADAAALEHDKAYDQQLKAGDNPYLKYNHA |
| underlined, starts | | DAEFQERLKEDTSFGGNLGRAVFQAKKRLLEPLGLVEEAAKTAPGKKR |
| at position 590 | | PVEQSPQEPDSSAGIGKSGAQPAKKRLNFGQTGDTESVPDPQPIGEPP |
| (immediately | | AAPSGVGSLTMASGGGAPVADNNEGADGVGSSSGNWHCDSQWLGDRVI |
| subsequent to | | TTSTRTWALPTYNNHLYKQISNSTSGGSSNDNAYFGYSTPWGYFDFNR |
| position 589); | | FHCHFSPRDWQRLINNNWGFRPKRLNFKLFNIQVKEVTDNNGVKTIAN |
| 743 aa | | NLTSTVQVFTDSDYQLPYVLGSAHEGCLPPFPADVFMIPQYGYLTLND |
| | | GSQAVGRSSFYCLEYFPSQMLRTGNNFQFSYEFENVPFHSSYAHSQSL |
| | | DRLMNPLIDQYLYYLSKTINGSGQNQQTLKFSVAGPSNMAVQGRNYIP |
| | | GPSYRQQRVSTTVTQNNNSEFAWPGASSWALNGRNSLMNPGPAMASHK |
| | | EGEDRFFPLSGSLIFGKQGTGRDNVDADKVMITNEEEIKTINPVATES |
| | | YGQVATNHQSAQAYSTDVRMQAQTGWVQNQGILPGMVWQDRDVYLQGP |
| | | IWAKIPHTDGNFHPSPLMGGFGMKHPPPQILIKNTPVPADPPTAFNKD |
| | | KLNSFITQYSTGQVSVEIEWELQKENSKRWNPEIQYTSNYYKSNNVEF |
| | | AVNTEGVYSEPRPIGTRYLIRNL |
| TTD-004 | 3639 | MAADGYLPDWLEDNLSEGIREWWALKPGAPQPKANQQHQDNARGLVLP |
| 7mer peptide | | GYKYLGPGNGLDKGEPVNAADAAALEHDKAYDQQLKAGDNPYLKYNHA |
| underlined, starts | | DAEFQERLKEDTSFGGNLGRAVFQAKKRLLEPLGLVEEAAKTAPGKKR |
| at position 589 | | PVEQSPQEPDSSAGIGKSGAQPAKKRLNFGQTGDTESVPDPQPIGEPP |
| (immediately | | AAPSGVGSLTMASGGGAPVADNNEGADGVGSSSGNWHCDSQWLGDRVI |
| subsequent to | | TTSTRTWALPTYNNHLYKQISNSTSGGSSNDNAYFGYSTPWGYFDFNR |
| position 588); | | FHCHFSPRDWQRLINNNWGFRPKRLNFKLFNIQVKEVIDNNGVKTIAN |
| 743 aa | | NLTSTVQVFTDSDYQLPYVLGSAHEGCLPPFPADVFMIPQYGYLTLND |
| | | GSQAVGRSSFYCLEYFPSQMLRIGNNFQFSYEFENVPFHSSYAHSQSL |
| | | DRLMNPLIDQYLYYLSKTINGSGQNQQTLKFSVAGPSNMAVQGRNYIP |
| | | GPSYRQQRVSTTVTQNNNSEFAWPGASSWALNGRNSLMNPGPAMASHK |
| | | EGEDRFFPLSGSLIFGKQGTGRDNVDADKVMITNEEEIKTINPVATES |
| | | YGQVATNHQSAQIVMNSLKAQAQTGWVQNQGILPGMVWQDRDVYLQGP |
| | | IWAKIPHTDGNFHPSPLMGGFGMKHPPPQILIKNTPVPADPPTAFNKD |
| | | KLNSFITQYSTGQVSVEIEWELQKENSKRWNPEIQYTSNYYKSNNVEF |
| | | AVNTEGVYSEPRPIGTRYLTRNL |
| TTD-005 | 3640 | MAADGYLPDWLEDNLSEGIREWWALKPGAPQPKANQQHQDNARGLVLP |
| 7mer peptide | | GYKYLGPGNGLDKGEPVNAADAAALEHDKAYDQQLKAGDNPYLKYNHA |
| underlined, starts | | DAEFQERLKEDTSFGGNLGRAVFQAKKRLLEPLGLVEEAAKTAPGKKR |
| at position 590 | | PVEQSPQEPDSSAGIGKSGAQPAKKRLNFGQTGDTESVPDPQPIGEPP |
| (immediately | | AAPSGVGSLTMASGGGAPVADNNEGADGVGSSSGNWHCDSQWLGDRVI |
| subsequent to | | TTSTRTWALPTYNNHLYKQISNSTSGGSSNDNAYFGYSTPWGYFDFNR |
| position 589); | | FHCHFSPRDWQRLINNNWGFRPKRLNFKLFNIQVKEVTDNNGVKTIAN |
| 743 aa | | NLTSTVQVFTDSDYQLPYVLGSAHEGCLPPFPADVFMIPQYGYLTLND |
| | | GSQAVGRSSFYCLEYFPSQMLRTGNNFQFSYEFENVPFHSSYAHSQSL |
| | | DRLMNPLIDQYLYYLSKTINGSGQNQQTLKFSVAGPSNMAVQGRNYIP |
| | | GPSYRQQRVSTTVTQNNNSEFAWPGASSWALNGRNSLMNPGPAMASHK |
| | | EGEDRFFPLSGSLIFGKQGTGRDNVDADKVMITNEEEIKTTNPVATES |
| | | YGQVATNHQSAQARESPRGLQAQTGWVQNQGILPGMVWQDRDVYLQGP |
| | | IWAKIPHTDGNFHPSPLMGGFGMKHPPPQILIKNTPVPADPPTAFNKD |
| | | KLNSFITQYSTGQVSVEIEWELQKENSKRWNPEIQYTSNYYKSNNVEF |
| | | AVNTEGVYSEPRPIGTRYLTRNL |
| TTD-006 | 3641 | MAADGYLPDWLEDNLSEGIREWWALKPGAPQPKANQQHQDNARGLVLP |
| 7mer peptide | | GYKYLGPGNGLDKGEPVNAADAAALEHDKAYDQQLKAGDNPYLKYNHA |
| underlined, starts | | DAEFQERLKEDTSFGGNLGRAVFQAKKRLLEPLGLVEEAAKTAPGKKR |
| at position 590 | | PVEQSPQEPDSSAGIGKSGAQPAKKRLNFGQTGDTESVPDPQPIGEPP |
| (immediately | | AAPSGVGSLIMASGGGAPVADNNEGADGVGSSSGNWHCDSQWLGDRVI |
| subsequent to | | TTSTRTWALPTYNNHLYKQISNSTSGGSSNDNAYFGYSTPWGYFDFNR |
| 589); 743 aa | | FHCHFSPRDWQRLINNNWGFRPKRLNFKLFNIQVKEVTDNNGVKTIAN |
| | | NLTSTVQVFTDSDYQLPYVLGSAHEGCLPPFPADVFMIPQYGYLTLND |
| | | GSQAVGRSSFYCLEYFPSQMLRTGNNFQFSYEFENVPFHSSYAHSQSL |
| | | DRLMNPLIDQYLYYLSKTINGSGQNQQTLKFSVAGPSNMAVQGRNYIP |
| | | GPSYRQQRVSTTVTQNNNSEFAWPGASSWALNGRNSLMNPGPAMASHK |
| | | EGEDRFFPLSGSLIFGKQGTGRDNVDADKVMITNEEEIKTTNPVATES |
| | | YGQVATNHQSAQASFNDTRAQAQTGWVQNQGILPGMVWQDRDVYLQGP |
| | | IWAKIPHTDGNFHPSPLMGGFGMKHPPPQILIKNTPVPADPPTAFNKD |
| | | KLNSFITQYSTGQVSVEIEWELQKENSKRWNPEIQYTSNYYKSNNVEF |
| | | AVNTEGVYSEPRPIGTRYLTRNL |
| TTD-007 | 3642 | MAADGYLPDWLEDNLSEGIREWWALKPGAPQPKANQQHQDNARGLVLP |
| 9mer peptide | | GYKYLGPGNGLDKGEPVNAADAAALEHDKAYDQQLKAGDNPYLKYNHA |
| underlined, starts | | DAEFQERLKEDTSFGGNLGRAVFQAKKRLLEPLGLVEEAAKTAPGKKR |
| at position 587 | | PVEQSPQEPDSSAGIGKSGAQPAKKRLNFGQTGDTESVPDPQPIGEPP |
| (immediately | | AAPSGVGSLTMASGGGAPVADNNEGADGVGSSSGNWHCDSQWLGDRVI |
| subsequent to | | TTSTRTWALPTYNNHLYKQISNSTSGGSSNDNAYFGYSTPWGYFDENR |
| position 586); | | FHCHFSPRDWQRLINNNWGFRPKRLNFKLFNIQVKEVTDNNGVKTIAN |
| 743 aa | | NLTSTVQVFTDSDYQLPYVLGSAHEGCLPPFPADVFMIPQYGYLILND |
| | | GSQAVGRSSFYCLEYFPSQMLRTGNNFQFSYEFENVPFHSSYAHSQSL |
| | | DRLMNPLIDQYLYYLSKTINGSGQNQQTLKFSVAGPSNMAVQGRNYIP |
| | | GPSYRQQRVSTTVTQNNNSEFAWPGASSWALNGRNSLMNPGPAMASHK |
| | | EGEDRFFPLSGSLIFGKQGTGRDNVDADKVMITNEEEIKTTNPVATES |
| | | YGQVATNHQSGGTLAVVSLAQAQTGWVQNQGILPGMVWQDRDVYLQGP |
| | | IWAKIPHTDGNFHPSPLMGGFGMKHPPPQILIKNTPVPADPPTAFNKD |
| | | KLNSFITQYSTGQVSVEIEWELQKENSKRWNPEIQYTSNYYKSNNVEF |
| | | AVNTEGVYSEPRPIGTRYLIRNL |
| TTD-008 | 3643 | MAADGYLPDWLEDNLSEGIREWWALKPGAPQPKANQQHQDNARGLVLP |
| 7mer peptide | | GYKYLGPGNGLDKGEPVNAADAAALEHDKAYDQQLKAGDNPYLKYNHA |
| underlined, starts | | DAEFQERLKEDTSFGGNLGRAVFQAKKRLLEPLGLVEEAAKTAPGKKR |
| at position 590 | | PVEQSPQEPDSSAGIGKSGAQPAKKRLNFGQTGDTESVPDPQPIGEPP |
| (immediately | | AAPSGVGSLTMASGGGAPVADNNEGADGVGSSSGNWHCDSQWLGDRVI |
| subsequent to | | TTSTRTWALPTYNNHLYKQISNSTSGGSSNDNAYFGYSTPWGYFDFNR |
| 589); 743 aa | | FHCHFSPRDWQRLINNNWGFRPKRLNFKLFNIQVKEVTDNNGVKTIAN |
| | | NLTSTVQVFTDSDYQLPYVLGSAHEGCLPPFPADVFMIPQYGYLTLND |
| | | GSQAVGRSSFYCLEYFPSQMLRTGNNFQFSYEFENVPFHSSYAHSQSL |
| | | DRLMNPLIDQYLYYLSKTINGSGQNQQTLKFSVAGPSNMAVQGRNYIP |
| | | GPSYRQQRVSTTVTQNNNSEFAWPGASSWALNGRNSLMNPGPAMASHK |
| | | EGEDRFFPLSGSLIFGKQGTGRDNVDADKVMITNEEEIKTTNPVATES |
| | | YGQVATNHQSAQAYGLPKGPQAQTGWVQNQGILPGMVWQDRDVYLQGP |
| | | IWAKIPHTDGNFHPSPLMGGFGMKHPPPQILIKNTPVPADPPTAFNKD |
| | | KLNSFITQYSTGQVSVEIEWELQKENSKRWNPEIQYTSNYYKSNNVEF |
| | | AVNTEGVYSEPRPIGTRYLTRNL |
| TTD-009 | 3644 | MAADGYLPDWLEDNLSEGIREWWALKPGAPQPKANQQHQDNARGLVLP |
| 7mer peptide | | GYKYLGPGNGLDKGEPVNAADAAALEHDKAYDQQLKAGDNPYLKYNHA |
| underlined, starts | | DAEFQERLKEDTSFGGNLGRAVFQAKKRLLEPLGLVEEAAKTAPGKKR |
| at position 590 | | PVEQSPQEPDSSAGIGKSGAQPAKKRLNFGQTGDTESVPDPQPIGEPP |
| (immediately | | AAPSGVGSLTMASGGGAPVADNNEGADGVGSSSGNWHCDSQWLGDRVI |
| subsequent to | | TTSTRTWALPTYNNHLYKQISNSTSGGSSNDNAYFGYSTPWGYFDFNR |
| 589); 743 aa | | FHCHFSPRDWQRLINNNWGFRPKRLNFKLFNIQVKEVTDNNGVKTIAN |
| | | NLTSTVQVFTDSDYQLPYVLGSAHEGCLPPFPADVFMIPQYGYLTLND |
| | | GSQAVGRSSFYCLEYFPSQMLRTGNNFQFSYEFENVPFHSSYAHSQSL |
| | | DRLMNPLIDQYLYYLSKTINGSGQNQQTLKFSVAGPSNMAVQGRNYIP |
| | | GPSYRQQRVSTTVTQNNNSEFAWPGASSWALNGRNSLMNPGPAMASHK |
| | | EGEDRFFPLSGSLIFGKQGTGRDNVDADKVMITNEEEIKITNPVATES |
| | | YGQVATNHQSAQASTGTLRLQAQTGWVQNQGILPGMVWQDRDVYLQGP |
| | | IWAKIPHTDGNFHPSPLMGGFGMKHPPPQILIKNTPVPADPPTAFNKD |
| | | KLNSFITQYSTGQVSVEIEWELQKENSKRWNPEIQYTSNYYKSNNVEF |
| | | AVNTEGVYSEPRPIGTRYLIRNL |
| TTD-010 | 3645 | MAADGYLPDWLEDNLSEGIREWWALKPGAPQPKANQQHQDNARGLVLP |
| 7mer peptide | | GYKYLGPGNGLDKGEPVNAADAAALEHDKAYDQQLKAGDNPYLKYNHA |
| underlined, starts | | DAEFQERLKEDTSFGGNLGRAVFQAKKRLLEPLGLVEEAAKTAPGKKR |
| at position 590 | | PVEQSPQEPDSSAGIGKSGAQPAKKRLNFGQTGDTESVPDPQPIGEPP |
| (immediately | | AAPSGVGSLTMASGGGAPVADNNEGADGVGSSSGNWHCDSQWLGDRVI |
| subsequent to | | TTSTRTWALPTYNNHLYKQISNSTSGGSSNDNAYFGYSTPWGYFDENR |
| 589); 743 aa | | FHCHFSPRDWQRLINNNWGFRPKRLNFKLFNIQVKEVTDNNGVKTIAN |
| | | NLTSTVQVFTDSDYQLPYVLGSAHEGCLPPFPADVFMIPQYGYLTLND |
| | | GSQAVGRSSFYCLEYFPSQMLRTGNNFQFSYEFENVPFHSSYAHSQSL |
| | | DRLMNPLIDQYLYYLSKTINGSGQNQQTLKFSVAGPSNMAVQGRNYIP |
| | | GPSYRQQRVSTTVTQNNNSEFAWPGASSWALNGRNSLMNPGPAMASHK |
| | | EGEDRFFPLSGSLIFGKQGTGRDNVDADKVMITNEEEIKTTNPVATES |
| | | YGQVATNHQSAQAYSTDERMQAQTGWVQNQGILPGMVWQDRDVYLQGP |
| | | IWAKIPHTDGNFHPSPLMGGFGMKHPPPQILIKNTPVPADPPTAFNKD |
| | | KLNSFITQYSTGQVSVEIEWELQKENSKRWNPEIQYTSNYYKSNNVEF |
| | | AVNTEGVYSEPRPIGTRYLTRNL |
| TTD-011 | 3646 | MAADGYLPDWLEDNLSEGIREWWALKPGAPQPKANQQHQDNARGLVLP |
| 7mer peptide | | GYKYLGPGNGLDKGEPVNAADAAALEHDKAYDQQLKAGDNPYLKYNHA |
| underlined, starts | | DAEFQERLKEDTSFGGNLGRAVFQAKKRLLEPLGLVEEAAKTAPGKKR |
| at position 590 | | PVEQSPQEPDSSAGIGKSGAQPAKKRLNFGQTGDTESVPDPQPIGEPP |
| (immediately | | AAPSGVGSLTMASGGGAPVADNNEGADGVGSSSGNWHCDSQWLGDRVI |
| subsequent to | | TTSTRTWALPTYNNHLYKQISNSTSGGSSNDNAYFGYSTPWGYFDFNR |
| 589); 743 aa | | FHCHFSPRDWQRLINNNWGFRPKRLNFKLFNIQVKEVTDNNGVKTIAN |
| | | NLTSTVQVFTDSDYQLPYVLGSAHEGCLPPFPADVFMIPQYGYLTLND |
| | | GSQAVGRSSFYCLEYFPSQMLRTGNNFQFSYEFENVPFHSSYAHSQSL |
| | | DRLMNPLIDQYLYYLSKTINGSGQNQQTLKFSVAGPSNMAVQGRNYIP |
| | | GPSYRQQRVSTTVTQNNNSEFAWPGASSWALNGRNSLMNPGPAMASHK |
| | | EGEDRFFPLSGSLIFGKQGTGRDNVDADKVMITNEEEIKTTNPVATES |
| | | YGQVATNHQSAQAYSTDERKQAQTGWVQNQGILPGMVWQDRDVYLQGP |
| | | IWAKIPHTDGNFHPSPLMGGFGMKHPPPQILIKNTPVPADPPTAFNKD |
| | | KLNSFITQYSTGQVSVEIEWELQKENSKRWNPEIQYTSNYYKSNNVEF |
| | | AVNTEGVYSEPRPIGTRYLTRNL |
| TTD-012 | 3647 | MAADGYLPDWLEDNLSEGIREWWALKPGAPQPKANQQHQDNARGLVLP |
| 7mer peptide | | GYKYLGPGNGLDKGEPVNAADAAALEHDKAYDQQLKAGDNPYLKYNHA |
| underlined, starts | | DAEFQERLKEDTSFGGNLGRAVFQAKKRLLEPLGLVEEAAKTAPGKKR |
| at position 590 | | PVEQSPQEPDSSAGIGKSGAQPAKKRLNFGQTGDTESVPDPQPIGEPP |
| (immediately | | AAPSGVGSLTMASGGGAPVADNNEGADGVGSSSGNWHCDSQWLGDRVI |
| subsequent to | | TTSTRTWALPTYNNHLYKQISNSTSGGSSNDNAYFGYSTPWGYFDFNR |
| 589); 743 aa | | FHCHFSPRDWQRLINNNWGFRPKRLNFKLFNIQVKEVTDNNGVKTIAN |
| | | NLTSTVQVFTDSDYQLPYVLGSAHEGCLPPFPADVFMIPQYGYLTLND |
| | | GSQAVGRSSFYCLEYFPSQMLRTGNNFQFSYEFENVPFHSSYAHSQSL |
| | | DRLMNPLIDQYLYYLSKTINGSGQNQQTLKFSVAGPSNMAVQGRNYIP |
| | | GPSYRQQRVSTTVTQNNNSEFAWPGASSWALNGRNSLMNPGPAMASHK |
| | | EGEDRFFPLSGSLIFGKQGTGRDNVDADKVMITNEEEIKTTNPVATES |
| | | YGQVATNHQSAQAYVSSVKMQAQTGWVQNQGILPGMVWQDRDVYLQGP |
| | | IWAKIPHTDGNFHPSPLMGGFGMKHPPPQILIKNTPVPADPPTAFNKD |
| | | KLNSFITQYSTGQVSVEIEWELQKENSKRWNPEIQYTSNYYKSNNVEF |
| | | AVNTEGVYSEPRPIGTRYLTRNL |
| TTD-013 | 5 | MAADGYLPDWLEDNLSEGIREWWALKPGAPQPKANQQHQDNARGLVLP |
| 9mer peptide | | GYKYLGPGNGLDKGEPVNAADAAALEHDKAYDQQLKAGDNPYLKYNHA |
| underlined, starts at | | DAEFQERLKEDTSFGGNLGRAVFQAKKRLLEPLGLVEEAAKTAPGKKR |
| position 587 | | PVEQSPQEPDSSAGIGKSGAQPAKKRLNFGQTGDTESVPDPQPIGEPP |
| (immediately | | AAPSGVGSLTMASGGGAPVADNNEGADGVGSSSGNWHCDSQWLGDRVI |
| subsequent to | | TTSTRTWALPTYNNHLYKQISNSTSGGSSNDNAYFGYSTPWGYFDFNR |
| position 586); | | FHCHFSPRDWQRLINNNWGFRPKRLNFKLFNIQVKEVTDNNGVKTIAN |
| modification at | | NLTSTVQVFTDSDYQLPYVLGSAHEGCLPPFPADVFMIPQYGYLTLND |
| position 604; | | GSQAVGRSSFYCLEYFPSQMLRTGNNFQFSYEFENVPFHSSYAHSQSL |
| 743 aa | | DRLMNPLIDQYLYYLSKTINGSGQNQQTLKFSVAGPSNMAVQGRNYIP |
| | | GPSYRQQRVSTTVTQNNNSEFAWPGASSWALNGRNSLMNPGPAMASHK |
| | | EGEDRFFPLSGSLIFGKQGTGRDNVDADKVMITNEEEIKTINPVATES |
| | | YGQVATNHQSPLNGAVHLYAQAQTGWVPNQGILPGMVWQDRDVYLQGP |
| | | IWAKIPHTDGNFHPSPLMGGFGMKHPPPQILIKNTPVPADPPTAFNKD |
| | | KLNSFITQYSTGQVSVEIEWELQKENSKRWNPEIQYTSNYYKSNNVEF |
| | | AVNTEGVYSEPRPIGTRYLTRNL |
| TTD-014 | 8 | MAADGYLPDWLEDNLSEGIREWWALKPGAPQPKANQQHQDNARGLVLP |
| 9mer peptide | | GYKYLGPGNGLDKGEPVNAADAAALEHDKAYDQQLKAGDNPYLKYNHA |
| underlined, starts at | | DAEFQERLKEDTSFGGNLGRAVFQAKKRLLEPLGLVEEAAKTAPGKKR |
| position 587 | | PVEQSPQEPDSSAGIGKSGAQPAKKRLNFGQTGDTESVPDPQPIGEPP |
| (immediately | | AAPSGVGSLTMASGGGAPVADNNEGADGVGSSSGNWHCDSQWLGDRVI |
| subsequent to | | TTSTRTWALPTYNNHLYKQISNSTSGGSSNDNAYFGYSTPWGYFDFNR |
| position 586); | | FHCHFSPRDWQRLINNNWGFRPKRLNFKLFNIQVKEVIDNNGVKTIAN |
| modifications at | | NLTSTVQVFTDSDYQLPYVLGSAHEGCLPPFPADVFMIPQYGYLTLND |
| positions 600, 601, | | GSQAVGRSSFYCLEYFPSQMLRTGNNFQFSYEFENVPFHSSYAHSQSL |
| 602, and 604 | | DRLMNPLIDQYLYYLSKTINGSGQNQQTLKFSVAGPSNMAVQGRNYIP |
| 743 aa | | GPSYRQQRVSTTVTQNNNSEFAWPGASSWALNGRNSLMNPGPAMASHK |
| | | EGEDRFFPLSGSLIFGKQGTGRDNVDADKVMITNEEEIKTTNPVATES |
| | | YGQVATNHQSPLNGAVHLYAQAQLSPVKNQGILPGMVWQDRDVYLQGP |
| | | IWAKIPHTDGNFHPSPLMGGFGMKHPPPQILIKNTPVPADPPTAFNKD |
| | | KLNSFITQYSTGQVSVEIEWELQKENSKRWNPEIQYTSNYYKSNNVEF |
| | | AVNTEGVYSEPRPIGTRYLTRNL |

TABLE 5
Exemplary full length capsid nucleic acid sequences

| | SEQ | |
|---|---|---|
| Name and | ID | |
| Annotation | NO: | NT Sequence |
| TTD-001 | 3623 | atggctgccgatggttatcttccagattggctcgaggacaaccttagtgaaggaa |
| 9mer | | ttcgcgagtggtgggctttgaaacctggagcccctcaacccaaggcaaatcaaca |
| peptide | | acatcaagacaacgctcgaggtcttgtgcttccgggttacaaataccttggaccc |
| underlined | | ggcaacggactcgacaagggggagccggtcaacgcagcagacgcggcggccctcg |
| | | agcacgacaaggcctacgaccagcagctcaaggccggagacaacccgtacctcaa |
| | | gtacaaccacgccgacgccgagttccaggagcggctcaaagaagatacgtctttt |
| | | gggggcaacctcgggcgagcagtcttccaggccaaaaagaggcttcttgaacctc |
| | | ttggtctggttgaggaagcggctaagacggctcctggaaagaagaggcctgtaga |
| | | gcagtctcctcaggaaccggactcctccgcgggtattggcaaatcgggtgcacag |
| | | cccgctaaaaagagactcaatttcggtcagactggcgacacagagtcagtcccag |
| | | accctcaaccaatcggagaacctcccgcagccccctcaggtgtgggatctcttac |
| | | aatggcttcaggtggtggcgcaccagtggcagacaataacgaaggtgccgatgga |
| | | gtgggtagttcctcgggaaattggcattgcgattcccaatggctgggggacagag |
| | | tcatcaccaccagcacccgaacctgggccctgcccacctacaacaatcacctcta |
| | | caagcaaatctccaacagcacatctggaggatcttcaaatgacaacgcctacttc |
| | | ggctacagcaccccctgggggtattttgacttcaacagattccactgccacttct |
| | | caccacgtgactggcagcgactcatcaacaacaactggggattccggcctaagcg |
| | | actcaacttcaagctcttcaacattcaggtcaaagaggttacggacaacaatgga |
| | | gtcaagaccatcgccaataaccttaccagcacggtccaggtcttcacggactcag |
| | | actatcagctcccgtacgtgctcgggtcggctcacgagggctgcctcccgccgtt |
| | | cccagcggacgttttcatgattcctcagtacgggtatctgacgcttaatgatgga |
| | | agccaggccgtgggtcgttcgtccttttactgcctggaatatttcccgtcgcaaa |
| | | tgctaagaacgggtaacaacttccagttcagctacgagtttgagaacgtaccttt |
| | | ccatagcagctacgctcacagccaaagcctggaccgactaatgaatccactcatc |
| | | gaccaatacttgtactatctctcaaagactattaacggttctggacagaatcaac |
| | | aaacgctaaaattcagtgtggccggacccagcaacatggctgtccagggaagaaa |
| | | ctacatacctggacccagctaccgacaacaacgtgtctcaaccactgtgactcaa |
| | | aacaacaacagcgaatttgcttggcctggagcttcttcttgggctctcaatggac |
| | | gtaatagcttgatgaatcctggacctgctatggccagccacaaagaaggagagga |
| | | ccgtttctttcctttgtctggatctttaatttttggcaaacaaggaactggaaga |
| | | gacaacgtggatgcggacaaagtcatgataaccaacgaagaagaaattaaaacta |
| | | ctaacccggtagcaacggagtcctatggacaagtggccacaaaccaccagagtcc |
| | | gcttaatggtgccgtccatctttatgctcaggcgcagaccggctgggttcaaaac |
| | | caaggaatacttccgggtatggtttggcaggacagagatgtgtacctgcaaggac |
| | | ccatttgggccaaaattcctcacacggacggcaactttcacccttctccgctgat |
| | | gggagggtttggaatgaagcacccgcctcctcagatcctcatcaaaaacacacct |
| | | gtacctgcCgatcctccaacggccttcaacaaggacaagctgaactctttcatca |
| | | cccagtattctactggccaagtcagcgtggagatcgagtgggagctgcagaagga |
| | | aaacagcaagcgGtggaacccggagatccagtacacttccaactattacaagtct |
| | | aataatgttgaatttgctgttaatactgaaggtgtatatagtgaaccccgcccca |
| | | ttggcaccagatacctgactcgtaatctgtaa |
| TTD-002 | 3624 | atggctgccgatggttatcttccagattggctcgaggacaaccttagtgaaggaa |
| 7mer | | ttcgcgagtggtgggctttgaaacctggagcccctcaacccaaggcaaatcaaca |
| peptide | | acatcaagacaacgctcgaggtcttgtgcttccgggttacaaataccttggaccc |
| underlined | | ggcaacggactcgacaagggggagccggtcaacgcagcagacgcggcggccctcg |
| | | agcacgacaaggcctacgaccagcagctcaaggccggagacaacccgtacctcaa |
| | | gtacaaccacgccgacgccgagttccaggagcggctcaaagaagatacgtctttt |
| | | gggggcaacctcgggcgagcagtcttccaggccaaaaagaggcttcttgaacctc |
| | | ttggtctggttgaggaagcggctaagacggctcctggaaagaagaggcctgtaga |
| | | gcagtctcctcaggaaccggactcctccgcgggtattggcaaatcgggtgcacag |
| | | cccgctaaaaagagactcaatttcggtcagactggcgacacagagtcagtcccag |
| | | accctcaaccaatcggagaacctcccgcagccccctcaggtgtgggatctcttac |
| | | aatggcttcaggtggtggcgcaccagtggcagacaataacgaaggtgccgatgga |
| | | gtgggtagttcctcgggaaattggcattgcgattcccaatggctgggggacagag |
| | | tcatcaccaccagcacccgaacctgggccctgcccacctacaacaatcacctcta |
| | | caagcaaatctccaacagcacatctggaggatcttcaaatgacaacgcctacttc |
| | | ggctacagcaccccctgggggtattttgacttcaacagattccactgccacttct |
| | | caccacgtgactggcagcgactcatcaacaacaactggggattccggcctaagcg |
| | | actcaacttcaagctcttcaacattcaggtcaaagaggttacggacaacaatgga |
| | | gtcaagaccatcgccaataaccttaccagcacggtccaggtcttcacggactcag |
| | | actatcagctcccgtacgtgctcgggtcggctcacgagggctgcctcccgccgtt |
| | | cccagcggacgttttcatgattcctcagtacgggtatctgacgcttaatgatgga |
| | | agccaggccgtgggtcgttcgtccttttactgcctggaatatttcccgtcgcaaa |
| | | tgctaagaacgggtaacaacttccagttcagctacgagtttgagaacgtaccttt |
| | | ccatagcagctacgctcacagccaaagcctggaccgactaatgaatccactcatc |
| | | gaccaatacttgtactatctctcaaagactattaacggttctggacagaatcaac |
| | | aaacgctaaaattcagtgtggccggacccagcaacatggctgtccagggaagaaa |
| | | ctacatacctggacccagctaccgacaacaacgtgtctcaaccactgtgactcaa |
| | | aacaacaacagcgaatttgcttggcctggagcttcttcttgggctctcaatggac |
| | | gtaatagcttgatgaatcctggacctgctatggccagccacaaagaaggagagga |
| | | ccgtttctttcctttgtctggatctttaatttttggcaaacaaggaactggaaga |
| | | gacaacgtggatgcggacaaagtcatgataaccaacgaagaagaaattaaaacta |
| | | ctaacccggtagcaacggagtcctatggacaagtggccacaaaccaccagagtgc |
| | | acaggctcgtgattctccgaagggttggcaggcgcagaccggctgggttcaaaac |
| | | caaggaatacttccgggtatggtttggcaggacagagatgtgtacctgcaaggac |
| | | ccatttgggccaaaattcctcacacggacggcaactttcacccttctccgctgat |
| | | gggagggtttggaatgaagcacccgcctcctcagatcctcatcaaaaacacacct |
| | | gtacctgcCgatcctccaacggccttcaacaaggacaagctgaactctttcatca |
| | | cccagtattctactggccaagtcagcgtggagatcgagtgggagctgcagaagga |
| | | aaacagcaagcgGtggaacccggagatccagtacacttccaactattacaagtct |
| | | aataatgttgaatttgctgttaatactgaaggtgtatatagtgaaccccgcccca |
| | | ttggcaccagatacctgactcgtaatctgtaa |
| TTD-003 | 3625 | atggctgccgatggttatcttccagattggctcgaggacaaccttagtgaaggaa |
| 7mer | | ttcgcgagtggtgggctttgaaacctggagcccctcaacccaaggcaaatcaaca |
| peptide | | acatcaagacaacgctcgaggtcttgtgcttccgggttacaaataccttggaccc |
| underlined | | ggcaacggactcgacaagggggagccggtcaacgcagcagacgcggcggccctcg |
| | | agcacgacaaggcctacgaccagcagctcaaggccggagacaacccgtacctcaa |
| | | gtacaaccacgccgacgccgagttccaggagcggctcaaagaagatacgtctttt |
| | | gggggcaacctcgggcgagcagtcttccaggccaaaaagaggcttcttgaacctc |
| | | ttggtctggttgaggaagcggctaagacggctcctggaaagaagaggcctgtaga |
| | | gcagtctcctcaggaaccggactcctccgcgggtattggcaaatcgggtgcacag |
| | | cccgctaaaaagagactcaatttcggtcagactggcgacacagagtcagtcccag |
| | | accctcaaccaatcggagaacctcccgcagccccctcaggtgtgggatctcttac |
| | | aatggcttcaggtggtggcgcaccagtggcagacaataacgaaggtgccgatgga |
| | | gtgggtagttcctcgggaaattggcattgcgattcccaatggctgggggacagag |
| | | tcatcaccaccagcacccgaacctgggccctgcccacctacaacaatcacctcta |
| | | caagcaaatctccaacagcacatctggaggatcttcaaatgacaacgcctacttc |
| | | ggctacagcaccccctgggggtattttgacttcaacagattccactgccacttct |
| | | caccacgtgactggcagcgactcatcaacaacaactggggattccggcctaagcg |
| | | actcaacttcaagctcttcaacattcaggtcaaagaggttacggacaacaatgga |
| | | gtcaagaccatcgccaataaccttaccagcacggtccaggtcttcacggactcag |
| | | actatcagctcccgtacgtgctcgggtcggctcacgagggctgcctcccgccgtt |
| | | cccagcggacgttttcatgattcctcagtacgggtatctgacgcttaatgatgga |
| | | agccaggccgtgggtcgttcgtccttttactgcctggaatatttcccgtcgcaaa |
| | | tgctaagaacgggtaacaacttccagttcagctacgagtttgagaacgtaccttt |
| | | ccatagcagctacgctcacagccaaagcctggaccgactaatgaatccactcatc |
| | | gaccaatacttgtactatctctcaaagactattaacggttctggacagaatcaac |
| | | aaacgctaaaattcagtgtggccggacccagcaacatggctgtccagggaagaaa |
| | | ctacatacctggacccagctaccgacaacaacgtgtctcaaccactgtgactcaa |
| | | aacaacaacagcgaatttgcttggcctggagcttcttcttgggctctcaatggac |
| | | gtaatagcttgatgaatcctggacctgctatggccagccacaaagaaggagagga |
| | | ccgtttctttcctttgtctggatctttaatttttggcaaacaaggaactggaaga |
| | | gacaacgtggatgcggacaaagtcatgataaccaacgaagaagaaattaaaacta |
| | | ctaacccggtagcaacggagtcctatggacaagtggccacaaaccaccagagtgc |
| | | acaggctcgtgattctccgaagggttggcaggcgcagaccggctgggttcaaaac |
| | | caaggaatacttccgggtatggtttggcaggacagagatgtgtacctgcaaggac |
| | | ccatttgggccaaaattcctcacacggacggcaactttcacccttctccgctgat |
| | | gggagggtttggaatgaagcacccgcctcctcagatcctcatcaaaaacacacct |
| | | gtacctgcggatcctccaacggccttcaacaaggacaagctgaactctttcatca |
| | | cccagtattctactggccaagtcagcgtggagatcgagtgggagctgcagaagga |
| | | aaacagcaagcgctggaacccggagatccagtacacttccaactattacaagtct |
| | | aataatgttgaatttgctgttaatactgaaggtgtatatagtgaaccccgcccca |
| | | ttggcaccagatacctgactcgtaatctgtaa |
| | 3626 | atggctgccgatggttatcttccagattggctcgaggacaaccttagtgaaggaa |
| | | ttcgcgagtggtgggctttgaaacctggagcccctcaacccaaggcaaatcaaca |
| | | acatcaagacaacgctcgaggtcttgtgcttccgggttacaaataccttggaccc |
| | | ggcaacggactcgacaagggggagccggtcaacgcagcagacgcggcggccctcg |
| | | agcacgacaaggcctacgaccagcagctcaaggccggagacaacccgtacctcaa |
| | | gtacaaccacgccgacgccgagttccaggagcggctcaaagaagatacgtctttt |
| | | gggggcaacctcgggcgagcagtcttccaggccaaaaagaggcttcttgaacctc |
| | | ttggtctggttgaggaagcggctaagacggctcctggaaagaagaggcctgtaga |
| | | gcagtctcctcaggaaccggactcctccgcgggtattggcaaatcgggtgcacag |
| | | cccgctaaaaagagactcaatttcggtcagactggcgacacagagtcagtcccag |
| | | accctcaaccaatcggagaacctcccgcagccccctcaggtgtgggatctcttac |
| | | aatggcttcaggtggtggcgcaccagtggcagacaataacgaaggtgccgatgga |
| | | gtgggtagttcctcgggaaattggcattgcgattcccaatggctgggggacagag |
| | | tcatcaccaccagcacccgaacctgggccctgcccacctacaacaatcacctcta |
| | | caagcaaatctccaacagcacatctggaggatcttcaaatgacaacgcctacttc |
| | | ggctacagcaccccctgggggtattttgacttcaacagattccactgccacttct |
| | | caccacgtgactggcagcgactcatcaacaacaactggggattccggcctaagcg |
| | | actcaacttcaagctcttcaacattcaggtcaaagaggttacggacaacaatgga |
| | | gtcaagaccatcgccaataaccttaccagcacggtccaggtcttcacggactcag |
| | | actatcagctcccgtacgtgctcgggtcggctcacgagggctgcctcccgccgtt |
| | | cccagcggacgttttcatgattcctcagtacgggtatctgacgcttaatgatgga |
| | | agccaggccgtgggtcgttcgtccttttactgcctggaatatttcccgtcgcaaa |
| | | tgctaagaacgggtaacaacttccagttcagctacgagtttgagaacgtaccttt |
| | | ccatagcagctacgctcacagccaaagcctggaccgactaatgaatccactcatc |
| | | gaccaatacttgtactatctctcaaagactattaacggttctggacagaatcaac |
| | | aaacgctaaaattcagtgtggccggacccagcaacatggctgtccagggaagaaa |
| | | ctacatacctggacccagctaccgacaacaacgtgtctcaaccactgtgactcaa |
| | | aacaacaacagcgaatttgcttggcctggagcttcttcttgggctctcaatggac |
| | | gtaatagcttgatgaatcctggacctgctatggccagccacaaagaaggagagga |
| | | ccgtttctttcctttgtctggatctttaatttttggcaaacaaggaactggaaga |
| | | gacaacgtggatgcggacaaagtcatgataaccaacgaagaagaaattaaaacta |
| | | ctaacccggtagcaacggagtcctatggacaagtggccacaaaccaccagagtgc |
| | | acaggcttattctacggatgtgaggatgcaggcgcagaccggctgggttcaaaac |
| | | caaggaatacttccgggtatggtttggcaggacagagatgtgtacctgcaaggac |
| | | ccatttgggccaaaattcctcacacggacggcaactttcacccttctccgctgat |
| | | gggagggtttggaatgaagcacccgcctcctcagatcctcatcaaaaacacacct |
| | | gtacctgcCgatcctccaacggccttcaacaaggacaagctgaactctttcatca |
| | | cccagtattctactggccaagtcagcgtggagatcgagtgggagctgcagaagga |
| | | aaacagcaagcgGtggaacccggagatccagtacacttccaactattacaagtct |
| | | aataatgttgaatttgctgttaatactgaaggtgtatatagtgaaccccgcccca |
| | | ttggcaccagatacctgactcgtaatctgtaa |
| TTD-004 | 3627 | atggctgccgatggttatcttccagattggctcgaggacaaccttagtgaaggaa |
| 7mer | | ttcgcgagtggtgggctttgaaacctggagcccctcaacccaaggcaaatcaaca |
| peptide | | acatcaagacaacgctcgaggtcttgtgcttccgggttacaaataccttggaccc |
| underlined | | ggcaacggactcgacaagggggagccggtcaacgcagcagacgcggcggccctcg |
| | | agcacgacaaggcctacgaccagcagctcaaggccggagacaacccgtacctcaa |
| | | gtacaaccacgccgacgccgagttccaggagcggctcaaagaagatacgtctttt |
| | | gggggcaacctcgggcgagcagtcttccaggccaaaaagaggcttcttgaacctc |
| | | ttggtctggttgaggaagcggctaagacggctcctggaaagaagaggcctgtaga |
| | | gcagtctcctcaggaaccggactcctccgcgggtattggcaaatcgggtgcacag |
| | | cccgctaaaaagagactcaatttcggtcagactggcgacacagagtcagtcccag |
| | | accctcaaccaatcggagaacctcccgcagccccctcaggtgtgggatctcttac |
| | | aatggcttcaggtggtggcgcaccagtggcagacaataacgaaggtgccgatgga |
| | | gtgggtagttcctcgggaaattggcattgcgattcccaatggctgggggacagag |
| | | tcatcaccaccagcacccgaacctgggccctgcccacctacaacaatcacctcta |
| | | caagcaaatctccaacagcacatctggaggatcttcaaatgacaacgcctacttc |
| | | ggctacagcaccccctgggggtattttgacttcaacagattccactgccacttct |
| | | caccacgtgactggcagcgactcatcaacaacaactggggattccggcctaagcg |
| | | actcaacttcaagctcttcaacattcaggtcaaagaggttacggacaacaatgga |
| | | gtcaagaccatcgccaataaccttaccagcacggtccaggtcttcacggactcag |
| | | actatcagctcccgtacgtgctcgggtcggctcacgagggctgcctcccgccgtt |
| | | cccagcggacgttttcatgattcctcagtacgggtatctgacgcttaatgatgga |
| | | agccaggccgtgggtcgttcgtccttttactgcctggaatatttcccgtcgcaaa |
| | | tgctaagaacgggtaacaacttccagttcagctacgagtttgagaacgtaccttt |
| | | ccatagcagctacgctcacagccaaagcctggaccgactaatgaatccactcatc |
| | | gaccaatacttgtactatctctcaaagactattaacggttctggacagaatcaac |
| | | aaacgctaaaattcagtgtggccggacccagcaacatggctgtccagggaagaaa |
| | | ctacatacctggacccagctaccgacaacaacgtgtctcaaccactgtgactcaa |
| | | aacaacaacagcgaatttgcttggcctggagcttcttcttgggctctcaatggac |
| | | gtaatagcttgatgaatcctggacctgctatggccagccacaaagaaggagagga |
| | | ccgtttctttcctttgtctggatctttaatttttggcaaacaaggaactggaaga |
| | | gacaacgtggatgcggacaaagtcatgataaccaacgaagaagaaattaaaacta |
| | | ctaacccggtagcaacggagtcctatggacaagtggccacaaaccaccagagtgc |
| | | acagattgttatgaattcgttgaaggctcaggcgcagaccggctgggttcaaaac |
| | | caaggaatacttccgggtatggtttggcaggacagagatgtgtacctgcaaggac |
| | | ccatttgggccaaaattcctcacacggacggcaactttcacccttctccgctgat |
| | | gggagggtttggaatgaagcacccgcctcctcagatcctcatcaaaaacacacct |
| | | gtacctgcCgatcctccaacggccttcaacaaggacaagctgaactctttcatca |
| | | cccagtattctactggccaagtcagcgtggagatcgagtgggagctgcagaagga |
| | | aaacagcaagcgGtggaacccggagatccagtacacttccaactattacaagtct |
| | | aataatgttgaatttgctgttaatactgaaggtgtatatagtgaaccccgcccca |
| | | ttggcaccagatacctgactcgtaatctgtaa |
| TTD-005 | 3628 | atggctgccgatggttatcttccagattggctcgaggacaaccttagtgaaggaa |
| 7mer | | ttcgcgagtggtgggctttgaaacctggagcccctcaacccaaggcaaatcaaca |
| peptide | | acatcaagacaacgctcgaggtcttgtgcttccgggttacaaataccttggaccc |
| underlined | | ggcaacggactcgacaagggggagccggtcaacgcagcagacgcggcggccctcg |
| | | agcacgacaaggcctacgaccagcagctcaaggccggagacaacccgtacctcaa |
| | | gtacaaccacgccgacgccgagttccaggagcggctcaaagaagatacgtctttt |
| | | gggggcaacctcgggcgagcagtcttccaggccaaaaagaggcttcttgaacctc |
| | | ttggtctggttgaggaagcggctaagacggctcctggaaagaagaggcctgtaga |
| | | gcagtctcctcaggaaccggactcctccgcgggtattggcaaatcgggtgcacag |
| | | cccgctaaaaagagactcaatttcggtcagactggcgacacagagtcagtcccag |
| | | accctcaaccaatcggagaacctcccgcagccccctcaggtgtgggatctcttac |
| | | aatggcttcaggtggtggcgcaccagtggcagacaataacgaaggtgccgatgga |
| | | gtgggtagttcctcgggaaattggcattgcgattcccaatggctgggggacagag |
| | | tcatcaccaccagcacccgaacctgggccctgcccacctacaacaatcacctcta |
| | | caagcaaatctccaacagcacatctggaggatcttcaaatgacaacgcctacttc |
| | | ggctacagcaccccctgggggtattttgacttcaacagattccactgccacttct |
| | | caccacgtgactggcagcgactcatcaacaacaactggggattccggcctaagcg |
| | | actcaacttcaagctcttcaacattcaggtcaaagaggttacggacaacaatgga |
| | | gtcaagaccatcgccaataaccttaccagcacggtccaggtcttcacggactcag |
| | | actatcagctcccgtacgtgctcgggtcggctcacgagggctgcctcccgccgtt |
| | | cccagcggacgttttcatgattcctcagtacgggtatctgacgcttaatgatgga |
| | | agccaggccgtgggtcgttcgtccttttactgcctggaatatttcccgtcgcaaa |
| | | tgctaagaacgggtaacaacttccagttcagctacgagtttgagaacgtaccttt |
| | | ccatagcagctacgctcacagccaaagcctggaccgactaatgaatccactcatc |
| | | gaccaatacttgtactatctctcaaagactattaacggttctggacagaatcaac |
| | | aaacgctaaaattcagtgtggccggacccagcaacatggctgtccagggaagaaa |
| | | ctacatacctggacccagctaccgacaacaacgtgtctcaaccactgtgactcaa |
| | | aacaacaacagcgaatttgcttggcctggagcttcttcttgggctctcaatggac |
| | | gtaatagcttgatgaatcctggacctgctatggccagccacaaagaaggagagga |
| | | ccgtttctttcctttgtctggatctttaatttttggcaaacaaggaactggaaga |
| | | gacaacgtggatgcggacaaagtcatgataaccaacgaagaagaaattaaaacta |
| | | ctaacccggtagcaacggagtcctatggacaagtggccacaaaccaccagagtgc |
| | | acaggctcgggagagtcctcgtgggctgcaggcgcagaccggctgggttcaaaac |
| | | caaggaatacttccgggtatggtttggcaggacagagatgtgtacctgcaaggac |
| | | ccatttgggccaaaattcctcacacggacggcaactttcacccttctccgctgat |
| | | gggagggtttggaatgaagcacccgcctcctcagatcctcatcaaaaacacacct |
| | | gtacctgcCgatcctccaacggccttcaacaaggacaagctgaactctttcatca |
| | | cccagtattctactggccaagtcagcgtggagatcgagtgggagctgcagaagga |
| | | aaacagcaagcgGtggaacccggagatccagtacacttccaactattacaagtct |
| | | aataatgttgaatttgctgttaatactgaaggtgtatatagtgaaccccgcccca |
| | | ttggcaccagatacctgactcgtaatctgtaa |
| TTD-006 | 3629 | atggctgccgatggttatcttccagattggctcgaggacaaccttagtgaaggaa |
| 7mer | | ttcgcgagtggtgggctttgaaacctggagcccctcaacccaaggcaaatcaaca |
| peptide | | acatcaagacaacgctcgaggtcttgtgcttccgggttacaaataccttggaccc |
| underlined | | ggcaacggactcgacaagggggagccggtcaacgcagcagacgcggcggccctcg |
| | | agcacgacaaggcctacgaccagcagctcaaggccggagacaacccgtacctcaa |
| | | gtacaaccacgccgacgccgagttccaggagcggctcaaagaagatacgtctttt |
| | | gggggcaacctcgggcgagcagtcttccaggccaaaaagaggcttcttgaacctc |
| | | ttggtctggttgaggaagcggctaagacggctcctggaaagaagaggcctgtaga |
| | | gcagtctcctcaggaaccggactcctccgcgggtattggcaaatcgggtgcacag |
| | | cccgctaaaaagagactcaatttcggtcagactggcgacacagagtcagtcccag |
| | | accctcaaccaatcggagaacctcccgcagccccctcaggtgtgggatctcttac |
| | | aatggcttcaggtggtggcgcaccagtggcagacaataacgaaggtgccgatgga |
| | | gtgggtagttcctcgggaaattggcattgcgattcccaatggctgggggacagag |
| | | tcatcaccaccagcacccgaacctgggccctgcccacctacaacaatcacctcta |
| | | caagcaaatctccaacagcacatctggaggatcttcaaatgacaacgcctacttc |
| | | ggctacagcaccccctgggggtattttgacttcaacagattccactgccacttct |
| | | caccacgtgactggcagcgactcatcaacaacaactggggattccggcctaagcg |
| | | actcaacttcaagctcttcaacattcaggtcaaagaggttacggacaacaatgga |
| | | gtcaagaccatcgccaataaccttaccagcacggtccaggtcttcacggactcag |
| | | actatcagctcccgtacgtgctcgggtcggctcacgagggctgcctcccgccgtt |
| | | cccagcggacgttttcatgattcctcagtacgggtatctgacgcttaatgatgga |
| | | agccaggccgtgggtcgttcgtccttttactgcctggaatatttcccgtcgcaaa |
| | | tgctaagaacgggtaacaacttccagttcagctacgagtttgagaacgtaccttt |
| | | ccatagcagctacgctcacagccaaagcctggaccgactaatgaatccactcatc |
| | | gaccaatacttgtactatctctcaaagactattaacggttctggacagaatcaac |
| | | aaacgctaaaattcagtgtggccggacccagcaacatggctgtccagggaagaaa |
| | | ctacatacctggacccagctaccgacaacaacgtgtctcaaccactgtgactcaa |
| | | aacaacaacagcgaatttgcttggcctggagcttcttcttgggctctcaatggac |
| | | gtaatagcttgatgaatcctggacctgctatggccagccacaaagaaggagagga |
| | | ccgtttctttcctttgtctggatctttaatttttggcaaacaaggaactggaaga |
| | | gacaacgtggatgcggacaaagtcatgataaccaacgaagaagaaattaaaacta |
| | | ctaacccggtagcaacggagtcctatggacaagtggccacaaaccaccagagtgc |
| | | acaggctagttttaatgatactagggctcaggcgcagaccggctgggttcaaaac |
| | | caaggaatacttccgggtatggtttggcaggacagagatgtgtacctgcaaggac |
| | | ccatttgggccaaaattcctcacacggacggcaactttcacccttctccgctgat |
| | | gggagggtttggaatgaagcacccgcctcctcagatcctcatcaaaaacacacct |
| | | gtacctgcCgatcctccaacggccttcaacaaggacaagctgaactctttcatca |
| | | cccagtattctactggccaagtcagcgtggagatcgagtgggagctgcagaagga |
| | | aaacagcaagcgGtggaacccggagatccagtacacttccaactattacaagtct |
| | | aataatgttgaatttgctgttaatactgaaggtgtatatagtgaaccccgcccca |
| | | ttggcaccagatacctgactcgtaatctgtaa |
| TTD-007 | 3630 | atggctgccgatggttatcttccagattggctcgaggacaaccttagtgaaggaa |
| 9mer | | ttcgcgagtggtgggctttgaaacctggagcccctcaacccaaggcaaatcaaca |
| peptide | | acatcaagacaacgctcgaggtcttgtgcttccgggttacaaataccttggaccc |
| underlined | | ggcaacggactcgacaagggggagccggtcaacgcagcagacgcggcggccctcg |
| | | agcacgacaaggcctacgaccagcagctcaaggccggagacaacccgtacctcaa |
| | | gtacaaccacgccgacgccgagttccaggagcggctcaaagaagatacgtctttt |
| | | gggggcaacctcgggcgagcagtcttccaggccaaaaagaggcttcttgaacctc |
| | | ttggtctggttgaggaagcggctaagacggctcctggaaagaagaggcctgtaga |
| | | gcagtctcctcaggaaccggactcctccgcgggtattggcaaatcgggtgcacag |
| | | cccgctaaaaagagactcaatttcggtcagactggcgacacagagtcagtcccag |
| | | accctcaaccaatcggagaacctcccgcagccccctcaggtgtgggatctcttac |
| | | aatggcttcaggtggtggcgcaccagtggcagacaataacgaaggtgccgatgga |
| | | gtgggtagttcctcgggaaattggcattgcgattcccaatggctgggggacagag |
| | | tcatcaccaccagcacccgaacctgggccctgcccacctacaacaatcacctcta |
| | | caagcaaatctccaacagcacatctggaggatcttcaaatgacaacgcctacttc |
| | | ggctacagcaccccctgggggtattttgacttcaacagattccactgccacttct |
| | | caccacgtgactggcagcgactcatcaacaacaactggggattccggcctaagcg |
| | | actcaacttcaagctcttcaacattcaggtcaaagaggttacggacaacaatgga |
| | | gtcaagaccatcgccaataaccttaccagcacggtccaggtcttcacggactcag |
| | | actatcagctcccgtacgtgctcgggtcggctcacgagggctgcctcccgccgtt |
| | | cccagcggacgttttcatgattcctcagtacgggtatctgacgcttaatgatgga |
| | | agccaggccgtgggtcgttcgtccttttactgcctggaatatttcccgtcgcaaa |
| | | tgctaagaacgggtaacaacttccagttcagctacgagtttgagaacgtaccttt |
| | | ccatagcagctacgctcacagccaaagcctggaccgactaatgaatccactcatc |
| | | gaccaatacttgtactatctctcaaagactattaacggttctggacagaatcaac |
| | | aaacgctaaaattcagtgtggccggacccagcaacatggctgtccagggaagaaa |
| | | ctacatacctggacccagctaccgacaacaacgtgtctcaaccactgtgactcaa |
| | | aacaacaacagcgaatttgcttggcctggagcttcttcttgggctctcaatggac |
| | | gtaatagcttgatgaatcctggacctgctatggccagccacaaagaaggagagga |
| | | ccgtttctttcctttgtctggatctttaatttttggcaaacaaggaactggaaga |
| | | gacaacgtggatgcggacaaagtcatgataaccaacgaagaagaaattaaaacta |
| | | ctaacccggtagcaacggagtcctatggacaagtggccacaaaccaccagagtgg |
| | | tggtacgttggccgtcgtgtcgcttgctcaggcgcagaccggctgggttcaaaac |
| | | caaggaatacttccgggtatggtttggcaggacagagatgtgtacctgcaaggac |
| | | ccatttgggccaaaattcctcacacggacggcaactttcacccttctccgctgat |
| | | gggagggtttggaatgaagcacccgcctcctcagatcctcatcaaaaacacacct |
| | | gtacctgcCgatcctccaacggccttcaacaaggacaagctgaactctttcatca |
| | | cccagtattctactggccaagtcagcgtggagatcgagtgggagctgcagaagga |
| | | aaacagcaagcgGtggaacccggagatccagtacacttccaactattacaagtct |
| | | aataatgttgaatttgctgttaatactgaaggtgtatatagtgaaccccgcccca |
| | | ttggcaccagatacctgactcgtaatctgtaa |
| TTD-008 | 3631 | atggctgccgatggttatcttccagattggctcgaggacaaccttagtgaaggaa |
| 7mer | | ttcgcgagtggtgggctttgaaacctggagcccctcaacccaaggcaaatcaaca |
| peptide | | acatcaagacaacgctcgaggtcttgtgcttccgggttacaaataccttggaccc |
| underlined | | ggcaacggactcgacaagggggagccggtcaacgcagcagacgcggcggccctcg |
| | | agcacgacaaggcctacgaccagcagctcaaggccggagacaacccgtacctcaa |
| | | gtacaaccacgccgacgccgagttccaggagcggctcaaagaagatacgtctttt |
| | | gggggcaacctcgggcgagcagtcttccaggccaaaaagaggcttcttgaacctc |
| | | ttggtctggttgaggaagcggctaagacggctcctggaaagaagaggcctgtaga |
| | | gcagtctcctcaggaaccggactcctccgcgggtattggcaaatcgggtgcacag |
| | | cccgctaaaaagagactcaatttcggtcagactggcgacacagagtcagtcccag |
| | | accctcaaccaatcggagaacctcccgcagccccctcaggtgtgggatctcttac |
| | | aatggcttcaggtggtggcgcaccagtggcagacaataacgaaggtgccgatgga |
| | | gtgggtagttcctcgggaaattggcattgcgattcccaatggctgggggacagag |
| | | tcatcaccaccagcacccgaacctgggccctgcccacctacaacaatcacctcta |
| | | caagcaaatctccaacagcacatctggaggatcttcaaatgacaacgcctacttc |
| | | ggctacagcaccccctgggggtattttgacttcaacagattccactgccacttct |
| | | caccacgtgactggcagcgactcatcaacaacaactggggattccggcctaagcg |
| | | actcaacttcaagctcttcaacattcaggtcaaagaggttacggacaacaatgga |
| | | gtcaagaccatcgccaataaccttaccagcacggtccaggtcttcacggactcag |
| | | actatcagctcccgtacgtgctcgggtcggctcacgagggctgcctcccgccgtt |
| | | cccagcggacgttttcatgattcctcagtacgggtatctgacgcttaatgatgga |
| | | agccaggccgtgggtcgttcgtccttttactgcctggaatatttcccgtcgcaaa |
| | | tgctaagaacgggtaacaacttccagttcagctacgagtttgagaacgtaccttt |
| | | ccatagcagctacgctcacagccaaagcctggaccgactaatgaatccactcatc |
| | | gaccaatacttgtactatctctcaaagactattaacggttctggacagaatcaac |
| | | aaacgctaaaattcagtgtggccggacccagcaacatggctgtccagggaagaaa |
| | | ctacatacctggacccagctaccgacaacaacgtgtctcaaccactgtgactcaa |
| | | aacaacaacagcgaatttgcttggcctggagcttcttcttgggctctcaatggac |
| | | gtaatagcttgatgaatcctggacctgctatggccagccacaaagaaggagagga |
| | | ccgtttctttcctttgtctggatctttaatttttggcaaacaaggaactggaaga |
| | | gacaacgtggatgcggacaaagtcatgataaccaacgaagaagaaattaaaacta |
| | | ctaacccggtagcaacggagtcctatggacaagtggccacaaaccaccagagtgc |
| | | acaggcttatgggttgccgaagggtcctcaggcgcagaccggctgggttcaaaac |
| | | caaggaatacttccgggtatggtttggcaggacagagatgtgtacctgcaaggac |
| | | ccatttgggccaaaattcctcacacggacggcaactttcacccttctccgctgat |
| | | gggagggtttggaatgaagcacccgcctcctcagatcctcatcaaaaacacacct |
| | | gtacctgcCgatcctccaacggccttcaacaaggacaagctgaactctttcatca |
| | | cccagtattctactggccaagtcagcgtggagatcgagtgggagctgcagaagga |
| | | aaacagcaagcgGtggaacccggagatccagtacacttccaactattacaagtct |
| | | aataatgttgaatttgctgttaatactgaaggtgtatatagtgaaccccgcccca |
| | | ttggcaccagatacctgactcgtaatctgtaa |
| TTD-009 | 3632 | atggctgccgatggttatcttccagattggctcgaggacaaccttagtgaaggaa |
| 7mer | | ttcgcgagtggtgggctttgaaacctggagcccctcaacccaaggcaaatcaaca |
| peptide | | acatcaagacaacgctcgaggtcttgtgcttccgggttacaaataccttggaccc |
| underlined | | ggcaacggactcgacaagggggagccggtcaacgcagcagacgcggcggccctcg |
| | | agcacgacaaggcctacgaccagcagctcaaggccggagacaacccgtacctcaa |
| | | gtacaaccacgccgacgccgagttccaggagcggctcaaagaagatacgtctttt |
| | | gggggcaacctcgggcgagcagtcttccaggccaaaaagaggcttcttgaacctc |
| | | ttggtctggttgaggaagcggctaagacggctcctggaaagaagaggcctgtaga |
| | | gcagtctcctcaggaaccggactcctccgcgggtattggcaaatcgggtgcacag |
| | | cccgctaaaaagagactcaatttcggtcagactggcgacacagagtcagtcccag |
| | | accctcaaccaatcggagaacctcccgcagccccctcaggtgtgggatctcttac |
| | | aatggcttcaggtggtggcgcaccagtggcagacaataacgaaggtgccgatgga |
| | | gtgggtagttcctcgggaaattggcattgcgattcccaatggctgggggacagag |
| | | tcatcaccaccagcacccgaacctgggccctgcccacctacaacaatcacctcta |
| | | caagcaaatctccaacagcacatctggaggatcttcaaatgacaacgcctacttc |
| | | ggctacagcaccccctgggggtattttgacttcaacagattccactgccacttct |
| | | caccacgtgactggcagcgactcatcaacaacaactggggattccggcctaagcg |
| | | actcaacttcaagctcttcaacattcaggtcaaagaggttacggacaacaatgga |
| | | gtcaagaccatcgccaataaccttaccagcacggtccaggtcttcacggactcag |
| | | actatcagctcccgtacgtgctcgggtcggctcacgagggctgcctcccgccgtt |
| | | cccagcggacgttttcatgattcctcagtacgggtatctgacgcttaatgatgga |
| | | agccaggccgtgggtcgttcgtccttttactgcctggaatatttcccgtcgcaaa |
| | | tgctaagaacgggtaacaacttccagttcagctacgagtttgagaacgtaccttt |
| | | ccatagcagctacgctcacagccaaagcctggaccgactaatgaatccactcatc |
| | | gaccaatacttgtactatctctcaaagactattaacggttctggacagaatcaac |
| | | aaacgctaaaattcagtgtggccggacccagcaacatggctgtccagggaagaaa |
| | | ctacatacctggacccagctaccgacaacaacgtgtctcaaccactgtgactcaa |
| | | aacaacaacagcgaatttgcttggcctggagcttcttcttgggctctcaatggac |
| | | gtaatagcttgatgaatcctggacctgctatggccagccacaaagaaggagagga |
| | | ccgtttctttcctttgtctggatctttaatttttggcaaacaaggaactggaaga |
| | | gacaacgtggatgcggacaaagtcatgataaccaacgaagaagaaattaaaacta |
| | | ctaacccggtagcaacggagtcctatggacaagtggccacaaaccaccagagtgc |
| | | acaggcttcgactgggacgcttcggcttcaggcgcagaccggctgggttcaaaac |
| | | caaggaatacttccgggtatggtttggcaggacagagatgtgtacctgcaaggac |
| | | ccatttgggccaaaattcctcacacggacggcaactttcacccttctccgctgat |
| | | gggagggtttggaatgaagcacccgcctcctcagatcctcatcaaaaacacacct |
| | | gtacctgcCgatcctccaacggccttcaacaaggacaagctgaactctttcatca |
| | | cccagtattctactggccaagtcagcgtggagatcgagtgggagctgcagaagga |
| | | aaacagcaagcgGtggaacccggagatccagtacacttccaactattacaagtct |
| | | aataatgttgaatttgctgttaatactgaaggtgtatatagtgaaccccgcccca |
| | | ttggcaccagatacctgactcgtaatctgtaa |
| TTD-010 | 3633 | atggctgccgatggttatcttccagattggctcgaggacaaccttagtgaaggaa |
| 7mer | | ttcgcgagtggtgggctttgaaacctggagcccctcaacccaaggcaaatcaaca |
| peptide | | acatcaagacaacgctcgaggtcttgtgcttccgggttacaaataccttggaccc |
| underlined | | ggcaacggactcgacaagggggagccggtcaacgcagcagacgcggcggccctcg |
| | | agcacgacaaggcctacgaccagcagctcaaggccggagacaacccgtacctcaa |
| | | gtacaaccacgccgacgccgagttccaggagcggctcaaagaagatacgtctttt |
| | | gggggcaacctcgggcgagcagtcttccaggccaaaaagaggcttcttgaacctc |
| | | ttggtctggttgaggaagcggctaagacggctcctggaaagaagaggcctgtaga |
| | | gcagtctcctcaggaaccggactcctccgcgggtattggcaaatcgggtgcacag |
| | | cccgctaaaaagagactcaatttcggtcagactggcgacacagagtcagtcccag |
| | | accctcaaccaatcggagaacctcccgcagccccctcaggtgtgggatctcttac |
| | | aatggcttcaggtggtggcgcaccagtggcagacaataacgaaggtgccgatgga |
| | | gtgggtagttcctcgggaaattggcattgcgattcccaatggctgggggacagag |
| | | tcatcaccaccagcacccgaacctgggccctgcccacctacaacaatcacctcta |
| | | caagcaaatctccaacagcacatctggaggatcttcaaatgacaacgcctacttc |
| | | ggctacagcaccccctgggggtattttgacttcaacagattccactgccacttct |
| | | caccacgtgactggcagcgactcatcaacaacaactggggattccggcctaagcg |
| | | actcaacttcaagctcttcaacattcaggtcaaagaggttacggacaacaatgga |
| | | gtcaagaccatcgccaataaccttaccagcacggtccaggtcttcacggactcag |
| | | actatcagctcccgtacgtgctcgggtcggctcacgagggctgcctcccgccgtt |
| | | cccagcggacgttttcatgattcctcagtacgggtatctgacgcttaatgatgga |
| | | agccaggccgtgggtcgttcgtccttttactgcctggaatatttcccgtcgcaaa |
| | | tgctaagaacgggtaacaacttccagttcagctacgagtttgagaacgtaccttt |
| | | ccatagcagctacgctcacagccaaagcctggaccgactaatgaatccactcatc |
| | | gaccaatacttgtactatctctcaaagactattaacggttctggacagaatcaac |
| | | aaacgctaaaattcagtgtggccggacccagcaacatggctgtccagggaagaaa |
| | | ctacatacctggacccagctaccgacaacaacgtgtctcaaccactgtgactcaa |
| | | aacaacaacagcgaatttgcttggcctggagcttcttcttgggctctcaatggac |
| | | gtaatagcttgatgaatcctggacctgctatggccagccacaaagaaggagagga |
| | | ccgtttctttcctttgtctggatctttaatttttggcaaacaaggaactggaaga |
| | | gacaacgtggatgcggacaaagtcatgataaccaacgaagaagaaattaaaacta |
| | | ctaacccggtagcaacggagtcctatggacaagtggccacaaaccaccagagtgc |
| | | gcaggcgtattcgacggatgagaggatgcaggcgcagaccggctgggttcaaaac |
| | | caaggaatacttccgggtatggtttggcaggacagagatgtgtacctgcaaggac |
| | | ccatttgggccaaaattcctcacacggacggcaactttcacccttctccgctgat |
| | | gggagggtttggaatgaagcacccgcctcctcagatcctcatcaaaaacacacct |
| | | gtacctgcCgatcctccaacggccttcaacaaggacaagctgaactctttcatca |
| | | cccagtattctactggccaagtcagcgtggagatcgagtgggagctgcagaagga |
| | | aaacagcaagcgGtggaacccggagatccagtacacttccaactattacaagtct |
| | | aataatgttgaatttgctgttaatactgaaggtgtatatagtgaaccccgcccca |
| | | ttggcaccagatacctgactcgtaatctgtaa |
| TTD-011 | 3634 | atggctgccgatggttatcttccagattggctcgaggacaaccttagtgaaggaa |
| 7mer | | ttcgcgagtggtgggctttgaaacctggagcccctcaacccaaggcaaatcaaca |
| peptide | | acatcaagacaacgctcgaggtcttgtgcttccgggttacaaataccttggaccc |
| underlined | | ggcaacggactcgacaagggggagccggtcaacgcagcagacgcggcggccctcg |
| | | agcacgacaaggcctacgaccagcagctcaaggccggagacaacccgtacctcaa |
| | | gtacaaccacgccgacgccgagttccaggagcggctcaaagaagatacgtctttt |
| | | gggggcaacctcgggcgagcagtcttccaggccaaaaagaggcttcttgaacctc |
| | | ttggtctggttgaggaagcggctaagacggctcctggaaagaagaggcctgtaga |
| | | gcagtctcctcaggaaccggactcctccgcgggtattggcaaatcgggtgcacag |
| | | cccgctaaaaagagactcaatttcggtcagactggcgacacagagtcagtcccag |
| | | accctcaaccaatcggagaacctcccgcagccccctcaggtgtgggatctcttac |
| | | aatggcttcaggtggtggcgcaccagtggcagacaataacgaaggtgccgatgga |
| | | gtgggtagttcctcgggaaattggcattgcgattcccaatggctgggggacagag |
| | | tcatcaccaccagcacccgaacctgggccctgcccacctacaacaatcacctcta |
| | | caagcaaatctccaacagcacatctggaggatcttcaaatgacaacgcctacttc |
| | | ggctacagcaccccctgggggtattttgacttcaacagattccactgccacttct |
| | | caccacgtgactggcagcgactcatcaacaacaactggggattccggcctaagcg |
| | | actcaacttcaagctcttcaacattcaggtcaaagaggttacggacaacaatgga |
| | | gtcaagaccatcgccaataaccttaccagcacggtccaggtcttcacggactcag |
| | | actatcagctcccgtacgtgctcgggtcggctcacgagggctgcctcccgccgtt |
| | | cccagcggacgttttcatgattcctcagtacgggtatctgacgcttaatgatgga |
| | | agccaggccgtgggtcgttcgtccttttactgcctggaatatttcccgtcgcaaa |
| | | tgctaagaacgggtaacaacttccagttcagctacgagtttgagaacgtaccttt |
| | | ccatagcagctacgctcacagccaaagcctggaccgactaatgaatccactcatc |
| | | gaccaatacttgtactatctctcaaagactattaacggttctggacagaatcaac |
| | | aaacgctaaaattcagtgtggccggacccagcaacatggctgtccagggaagaaa |
| | | ctacatacctggacccagctaccgacaacaacgtgtctcaaccactgtgactcaa |
| | | aacaacaacagcgaatttgcttggcctggagcttcttcttgggctctcaatggac |
| | | gtaatagcttgatgaatcctggacctgctatggccagccacaaagaaggagagga |
| | | ccgtttctttcctttgtctggatctttaatttttggcaaacaaggaactggaaga |
| | | gacaacgtggatgcggacaaagtcatgataaccaacgaagaagaaattaaaacta |
| | | ctaacccggtagcaacggagtcctatggacaagtggccacaaaccaccagagtgc |
| | | gcaggcgtattcgacggatgagaggaagcaggcgcagaccggctgggttcaaaac |
| | | caaggaatacttccgggtatggtttggcaggacagagatgtgtacctgcaaggac |
| | | ccatttgggccaaaattcctcacacggacggcaactttcacccttctccgctgat |
| | | gggagggtttggaatgaagcacccgcctcctcagatcctcatcaaaaacacacct |
| | | gtacctgcCgatcctccaacggccttcaacaaggacaagctgaactctttcatca |
| | | cccagtattctactggccaagtcagcgtggagatcgagtgggagctgcagaagga |
| | | aaacagcaagcgGtggaacccggagatccagtacacttccaactattacaagtct |
| | | aataatgttgaatttgctgttaatactgaaggtgtatatagtgaaccccgcccca |
| | | ttggcaccagatacctgactcgtaatctgtaa |
| TTD-012 | 3635 | atggctgccgatggttatcttccagattggctcgaggacaaccttagtgaaggaa |
| 7mer | | ttcgcgagtggtgggctttgaaacctggagcccctcaacccaaggcaaatcaaca |
| peptide | | acatcaagacaacgctcgaggtcttgtgcttccgggttacaaataccttggaccc |
| underlined | | ggcaacggactcgacaagggggagccggtcaacgcagcagacgcggcggccctcg |
| | | agcacgacaaggcctacgaccagcagctcaaggccggagacaacccgtacctcaa |
| | | gtacaaccacgccgacgccgagttccaggagcggctcaaagaagatacgtctttt |
| | | gggggcaacctcgggcgagcagtcttccaggccaaaaagaggcttcttgaacctc |
| | | ttggtctggttgaggaagcggctaagacggctcctggaaagaagaggcctgtaga |
| | | gcagtctcctcaggaaccggactcctccgcgggtattggcaaatcgggtgcacag |
| | | cccgctaaaaagagactcaatttcggtcagactggcgacacagagtcagtcccag |
| | | accctcaaccaatcggagaacctcccgcagccccctcaggtgtgggatctcttac |
| | | aatggcttcaggtggtggcgcaccagtggcagacaataacgaaggtgccgatgga |
| | | gtgggtagttcctcgggaaattggcattgcgattcccaatggctgggggacagag |
| | | tcatcaccaccagcacccgaacctgggccctgcccacctacaacaatcacctcta |
| | | caagcaaatctccaacagcacatctggaggatcttcaaatgacaacgcctacttc |
| | | ggctacagcaccccctgggggtattttgacttcaacagattccactgccacttct |
| | | caccacgtgactggcagcgactcatcaacaacaactggggattccggcctaagcg |
| | | actcaacttcaagctcttcaacattcaggtcaaagaggttacggacaacaatgga |
| | | gtcaagaccatcgccaataaccttaccagcacggtccaggtcttcacggactcag |
| | | actatcagctcccgtacgtgctcgggtcggctcacgagggctgcctcccgccgtt |
| | | cccagcggacgttttcatgattcctcagtacgggtatctgacgcttaatgatgga |
| | | agccaggccgtgggtcgttcgtccttttactgcctggaatatttcccgtcgcaaa |
| | | tgctaagaacgggtaacaacttccagttcagctacgagtttgagaacgtaccttt |
| | | ccatagcagctacgctcacagccaaagcctggaccgactaatgaatccactcatc |
| | | gaccaatacttgtactatctctcaaagactattaacggttctggacagaatcaac |
| | | aaacgctaaaattcagtgtggccggacccagcaacatggctgtccagggaagaaa |
| | | ctacatacctggacccagctaccgacaacaacgtgtctcaaccactgtgactcaa |
| | | aacaacaacagcgaatttgcttggcctggagcttcttcttgggctctcaatggac |
| | | gtaatagcttgatgaatcctggacctgctatggccagccacaaagaaggagagga |
| | | ccgtttctttcctttgtctggatctttaatttttggcaaacaaggaactggaaga |
| | | gacaacgtggatgcggacaaagtcatgataaccaacgaagaagaaattaaaacta |
| | | ctaacccggtagcaacggagtcctatggacaagtggccacaaaccaccagagtgc |
| | | acaggcttatgtttcgtctgttaagatgcaggcgcagaccggctgggttcaaaac |
| | | caaggaatacttccgggtatggtttggcaggacagagatgtgtacctgcaaggac |
| | | ccatttgggccaaaattcctcacacggacggcaactttcacccttctccgctgat |
| | | gggagggtttggaatgaagcacccgcctcctcagatcctcatcaaaaacacacct |
| | | gtacctgcCgatcctccaacggccttcaacaaggacaagctgaactctttcatca |
| | | cccagtattctactggccaagtcagcgtggagatcgagtgggagctgcagaagga |
| | | aaacagcaagcgGtggaacccggagatccagtacacttccaactattacaagtct |
| | | aataatgttgaatttgctgttaatactgaaggtgtatatagtgaaccccgcccca |
| | | ttggcaccagatacctgactcgtaatctgtaa |
| TTD-013 | 4 | ATGGCTGCCGATGGTTATCTTCCAGATTGGCTCGAGGACAACCTTAGTGAAGGAATTCGC |
| | | GAGTGGTGGGCTTTGAAACCTGGAGCCCCTCAACCCAAGGCAAATCAACAACATCAAGAC |
| | | AACGCTCGAGGTCTTGTGCTTCCGGGTTACAAATACCTTGGACCCGGCAACGGACTCGAC |
| | | AAGGGGGAGCCGGTCAACGCAGCAGACGCGGCGGCCCTCGAGCACGACAAGGCCTACGAC |
| | | CAGCAGCTCAAGGCCGGAGACAACCCGTACCTCAAGTACAACCACGCCGACGCCGAGTTC |
| | | CAGGAGCGGCTCAAAGAAGATACGTCTTTTGGGGGCAACCTCGGGCGAGCAGTCTTCCAG |
| | | GCCAAAAAGAGGCTTCTTGAACCTCTTGGTCTGGTTGAGGAAGCGGCTAAGACGGCTCCT |
| | | GGAAAGAAGAGGCCTGTAGAGCAGTCTCCTCAGGAACCGGACTCCTCCGCGGGTATTGGC |
| | | AAATCGGGTGCACAGCCCGCTAAAAAGAGACTCAATTTCGGTCAGACTGGCGACACAGAG |
| | | TCAGTCCCAGACCCTCAACCAATCGGAGAACCTCCCGCAGCCCCCTCAGGTGTGGGATCT |
| | | CTTACAATGGCTTCAGGTGGTGGCGCACCAGTGGCAGACAATAACGAAGGTGCCGATGGA |
| | | GTGGGTAGTTCCTCGGGAAATTGGCATTGCGATTCCCAATGGCTGGGGGACAGAGTCATC |
| | | ACCACCAGCACCCGAACCTGGGCCCTGCCCACCTACAACAATCACCTCTACAAGCAAATC |
| | | TCCAACAGCACATCTGGAGGATCTTCAAATGACAACGCCTACTTCGGCTACAGCACCCCC |
| | | TGGGGGTATTTTGACTTCAACAGATTCCACTGCCACTTCTCACCACGTGACTGGCAGCGA |
| | | CTCATCAACAACAACTGGGGATTCCGGCCTAAGCGACTCAACTTCAAGCTCTTCAACATT |
| | | CAGGTCAAAGAGGTTACGGACAACAATGGAGTCAAGACCATCGCCAATAACCTTACCAGC |
| | | ACGGTCCAGGTCTTCACGGACTCAGACTATCAGCTCCCGTACGTGCTCGGGTCGGCTCAC |
| | | GAGGGCTGCCTCCCGCCGTTCCCAGCGGACGTTTTCATGATTCCTCAGTACGGGTATCTG |
| | | ACGCTTAATGATGGAAGCCAGGCCGTGGGTCGTTCGTCCTTTTACTGCCTGGAATATTTC |
| | | CCGTCGCAAATGCTAAGAACGGGTAACAACTTCCAGTTCAGCTACGAGTTTGAGAACGTA |
| | | CCTTTCCATAGCAGCTACGCTCACAGCCAAAGCCTGGACCGACTAATGAATCCACTCATC |
| | | GACCAATACTTGTACTATCTCTCAAAGACTATTAACGGTTCTGGACAGAATCAACAAACG |
| | | CTAAAATTCAGTGTGGCCGGACCCAGCAACATGGCTGTCCAGGGAAGAAACTACATACCT |
| | | GGACCCAGCTACCGACAACAACGTGTCTCAACCACTGTGACTCAAAACAACAACAGCGAA |
| | | TTTGCTTGGCCTGGAGCTTCTTCTTGGGCTCTCAATGGACGTAATAGCTTGATGAATCCT |
| | | GGACCTGCTATGGCCAGCCACAAAGAAGGAGAGGACCGTTTCTTTCCTTTGTCTGGATCT |
| | | TTAATTTTTGGCAAACAAGGAACTGGAAGAGACAACGTGGATGCGGACAAAGTCATGATA |
| | | ACCAACGAAGAAGAAATTAAAACTACTAACCCGGTAGCAACGGAGTCCTATGGACAAGTG |
| | | GCCACAAACCACCAGAGTccgcttaatggtgccgtccatctttatGCTCAGGCGCAGACC |
| | | GGCTGGGTTccgAACCAAGGAATACTTCCGGGTATGGTTTGGCAGGACAGAGATGTGTAC |
| | | CTGCAAGGACCCATTTGGGCCAAAATTCCTCACACGGACGGCAACTTTCACCCTTCTCCG |
| | | CTGATGGGAGGGTTTGGAATGAAGCACCCGCCTCCTCAGATCCTCATCAAAAACACACCT |
| | | GTACCTGCCGATCCTCCAACGGCCTTCAACAAGGACAAGCTGAACTCTTTCATCACCCAG |
| | | TATTCTACTGGCCAAGTCAGCGTGGAGATCGAGTGGGAGCTGCAGAAGGAAAACAGCAAG |
| | | CGGTGGAACCCGGAGATCCAGTACACTTCCAACTATTACAAGTCTAATAATGTTGAATTT |
| | | GCTGTTAATACTGAAGGTGTATATAGTGAACCCCGCCCCATTGGCACCAGATACCTGACT |
| | | CGTAATCTGTAA |
| TTD-014 | 7 | ATGGCTGCCGATGGTTATCTTCCAGATTGGCTCGAGGACAACCTTAGTGAAGGAATTCGC |
| | | GAGTGGTGGGCTTTGAAACCTGGAGCCCCTCAACCCAAGGCAAATCAACAACATCAAGAC |
| | | AACGCTCGAGGTCTTGTGCTTCCGGGTTACAAATACCTTGGACCCGGCAACGGACTCGAC |
| | | AAGGGGGAGCCGGTCAACGCAGCAGACGCGGCGGCCCTCGAGCACGACAAGGCCTACGAC |
| | | CAGCAGCTCAAGGCCGGAGACAACCCGTACCTCAAGTACAACCACGCCGACGCCGAGTTC |
| | | CAGGAGCGGCTCAAAGAAGATACGTCTTTTGGGGGCAACCTCGGGCGAGCAGTCTTCCAG |
| | | GCCAAAAAGAGGCTTCTTGAACCTCTTGGTCTGGTTGAGGAAGCGGCTAAGACGGCTCCT |
| | | GGAAAGAAGAGGCCTGTAGAGCAGTCTCCTCAGGAACCGGACTCCTCCGCGGGTATTGGC |
| | | AAATCGGGTGCACAGCCCGCTAAAAAGAGACTCAATTTCGGTCAGACTGGCGACACAGAG |
| | | TCAGTCCCAGACCCTCAACCAATCGGAGAACCTCCCGCAGCCCCCTCAGGTGTGGGATCT |
| | | CTTACAATGGCTTCAGGTGGTGGCGCACCAGTGGCAGACAATAACGAAGGTGCCGATGGA |
| | | GTGGGTAGTTCCTCGGGAAATTGGCATTGCGATTCCCAATGGCTGGGGGACAGAGTCATC |
| | | ACCACCAGCACCCGAACCTGGGCCCTGCCCACCTACAACAATCACCTCTACAAGCAAATC |
| | | TCCAACAGCACATCTGGAGGATCTTCAAATGACAACGCCTACTTCGGCTACAGCACCCCC |
| | | TGGGGGTATTTTGACTTCAACAGATTCCACTGCCACTTCTCACCACGTGACTGGCAGCGA |
| | | CTCATCAACAACAACTGGGGATTCCGGCCTAAGCGACTCAACTTCAAGCTCTTCAACATT |
| | | CAGGTCAAAGAGGTTACGGACAACAATGGAGTCAAGACCATCGCCAATAACCTTACCAGC |
| | | ACGGTCCAGGTCTTCACGGACTCAGACTATCAGCTCCCGTACGTGCTCGGGTCGGCTCAC |
| | | GAGGGCTGCCTCCCGCCGTTCCCAGCGGACGTTTTCATGATTCCTCAGTACGGGTATCTG |
| | | ACGCTTAATGATGGAAGCCAGGCCGTGGGTCGTTCGTCCTTTTACTGCCTGGAATATTTC |
| | | CCGTCGCAAATGCTAAGAACGGGTAACAACTTCCAGTTCAGCTACGAGTTTGAGAACGTA |
| | | CCTTTCCATAGCAGCTACGCTCACAGCCAAAGCCTGGACCGACTAATGAATCCACTCATC |
| | | GACCAATACTTGTACTATCTCTCAAAGACTATTAACGGTTCTGGACAGAATCAACAAACG |
| | | CTAAAATTCAGTGTGGCCGGACCCAGCAACATGGCTGTCCAGGGAAGAAACTACATACCT |
| | | GGACCCAGCTACCGACAACAACGTGTCTCAACCACTGTGACTCAAAACAACAACAGCGAA |
| | | TTTGCTTGGCCTGGAGCTTCTTCTTGGGCTCTCAATGGACGTAATAGCTTGATGAATCCT |
| | | GGACCTGCTATGGCCAGCCACAAAGAAGGAGAGGACCGTTTCTTTCCTTTGTCTGGATCT |
| | | TTAATTTTTGGCAAACAAGGAACTGGAAGAGACAACGTGGATGCGGACAAAGTCATGATA |
| | | ACCAACGAAGAAGAAATTAAAACTACTAACCCGGTAGCAACGGAGTCCTATGGACAgGTc |
| | | GCtACGAATCATCAGTCTCCGCTGAATGGTGCGGTGCATCTGTATGCGCAGGCGCAGCTG |
| | | TCTCCGGTGAAGAATcaaggaatacttccgggtatgGTTTGGCAGGACAGAGATGTGTAC |
| | | CTGCAAGGACCCATTTGGGCCAAAATTCCTCACACGGACGGCAACTTTCACCCTTCTCCG |
| | | CTGATGGGAGGGTTTGGAATGAAGCACCCGCCTCCTCAGATCCTCATCAAAAACACACCT |
| | | GTACCTGCCGATCCTCCAACGGCCTTCAACAAGGACAAGCTGAACTCTTTCATCACCCAG |
| | | TATTCTACTGGCCAAGTCAGCGTGGAGATCGAGTGGGAGCTGCAGAAGGAAAACAGCAAG |
| | | CGGTGGAACCCGGAGATCCAGTACACTTCCAACTATTACAAGTCTAATAATGTTGAATTT |
| | | GCTGTTAATACTGAAGGTGTATATAGTGAACCCCGCCCCATTGGCACCAGATACCTGACT |
| | | CGTAATCTGTAA |

In some embodiments, an AAV capsid polypeptide, e.g., an AAV capsid variant, described herein comprises the amino acid sequence of any one of SEQ ID NOs: 3636-3647, or an amino acid sequence with at least 80% (e.g., at least about 85, 90, 95, 96, 97, 98, or 99%) sequence identity thereto. In some embodiments, an AAV capsid variant described herein comprises the amino acid sequence of SEQ ID NO: 3636, or an amino acid sequence with at least 80% (e.g., at least about 85, 90, 95, 96, 97, 98, or 99%) sequence identity thereto. In some embodiments, an AAV capsid variant described herein comprises the amino acid sequence of SEQ ID NO: 3639, or an amino acid sequence with at least 80% (e.g., at least about 85, 90, 95, 96, 97, 98, or 99%) sequence identity thereto.

In some embodiments, the polynucleotide encoding an AAV capsid polypeptide, e.g., AAV capsid variant, described herein comprises the nucleotide sequence of any one of SEQ ID NOs: 3623-3635, or a nucleotide sequence with at least 80% (e.g., at least about 85, 90, 95, 96, 97, 98, or 99%) sequence identity thereto. In some embodiments, the polynucleotide encoding an AAV capsid variant described herein comprises the nucleotide sequence of SEQ ID NO: 3623, or a nucleotide sequence with at least 80% (e.g., at least about 85, 90, 95, 96, 97, 98, or 99%) sequence identity thereto. In some embodiments, the polynucleotide encoding an AAV capsid variant described herein comprises the nucleotide sequence of SEQ ID NO: 3627, or a nucleotide sequence with at least 80% (e.g., at least about 85, 90, 95, 96, 97, 98, or 99%) sequence identity thereto. In some embodiments, the nucleic acid sequence encoding an AAV capsid polypeptide, e.g., an AAV capsid variant, described herein is codon optimized.

In some embodiments, the AAV capsid polypeptide, e.g., the AAV capsid variant, comprises a VP2 protein comprising the amino acid sequence corresponding to positions 138-743, of any one of SEQ ID NOs: 3636-3647, or a sequence with at least 80% (e.g., at least about 85, 90, 95, 96, 97, 98, or 99%) sequence identity thereto. In some embodiments, the AAV capsid comprises a VP3 protein comprising the amino acid sequence corresponding to positions 203-743, of any one of SEQ ID NOs: 3636-3647, or a sequence with at least 80% (e.g., at least about 85, 90, 95, 96, 97, 98, or 99%) sequence identity thereto.

In some embodiments, the AAV capsid polypeptide, e.g., the AAV capsid variant, e.g., an AAV capsid variant described herein, comprises the amino acid sequence of any one of SEQ ID NOs: 3636-3647, or an amino acid sequence with at least 80% (e.g., at least about 85, 90, 95, 96, 97, 98, or 99%) sequence identity thereto. In some embodiments, the AAV capsid polypeptide, e.g., the AAV capsid variant, e.g., an AAV capsid variant described herein, comprises an amino acid sequence comprising at least one, two, or three modifications, substitutions (e.g., conservative substitutions), insertions, or deletions, but not more than 30, 20 or 10 modifications, substitutions (e.g., conservative substitutions), insertions, or deletions relative to the amino acid sequence of any one of SEQ ID NOs: 3636-3647. In some embodiments, the AAV capsid polypeptide, e.g., the AAV capsid variant, e.g., an AAV capsid variant described herein, comprises an amino acid sequence having at least one, two, or three but no more than 30, 20, or 10 different amino acids relative to the amino acid sequence of any one of SEQ ID NOs: 3636-3647.

In some embodiments, the AAV capsid polypeptide, e.g., the AAV capsid variant, e.g., an AAV capsid variant described herein, comprises the amino acid sequence of SEQ ID NO: 3636, or an amino acid sequence with at least 80% (e.g., at least about 85, 90, 95, 96, 97, 98, or 99%) sequence identity thereto. In some embodiments, the AAV capsid polypeptide, e.g., the AAV capsid variant, e.g., an AAV capsid variant described herein, comprises an amino acid sequence comprising at least one, two, or three modifications, substitutions (e.g., conservative substitutions), insertions, or deletions, but not more than 30, 20 or 10 modifications, substitutions (e.g., conservative substitutions), insertions, or deletions relative to the amino acid sequence of SEQ ID NO: 3636. In some embodiments, the AAV capsid polypeptide, e.g., the AAV capsid variant, e.g., an AAV capsid variant described herein, comprises an amino acid sequence having at least one, two, or three but no more than 30, 20, or 10 different amino acids relative to the amino acid sequence of SEQ ID NO: 3636.

In some embodiments, an AAV capsid polypeptide, e.g., an AAV capsid variant, described herein has an increased tropism for a CNS cell or tissue, e.g., a brain cell, brain tissue, spinal cord cell, or spinal cord tissue, relative to the tropism of a reference sequence comprising the amino acid sequence of SEQ ID NO: 138.

In some embodiments, an AAV capsid polypeptide, e.g., an AAV capsid variant, described herein transduces a brain region, e.g., selected from dentate nucleus, cerebellar cortex, cerebral cortex, brain stem, hippocampus, thalamus and putamen. In some embodiments, the level of transduction of said brain region is at least 5, 10, 50, 100, 200, 500, 1,000, 2,000, 5,000, or 10,000-fold greater as compared to a reference sequence of SEQ ID NO: 138.

In some embodiments, an AAV capsid polypeptide, e.g., an AAV capsid variant, described herein is enriched at least about 5, 6, 7, 8, 9, or 10-fold, in the brain compared to a reference sequence of SEQ ID NO: 138. In some embodiments, an AAV capsid variant described herein is enriched at least about 20, 30, 40, or 50-fold in the brain compared to a reference sequence of SEQ ID NO: 138. In some embodiments, an AAV capsid variant described herein is enriched at least about 100, 200, 300, or 400-fold in the brain compared to a reference sequence of SEQ ID NO: 138.

In some embodiments, an AAV capsid polypeptide, e.g., an AAV capsid variant, described herein delivers an increased level of viral genomes to a brain region. In some embodiments, the level of viral genomes is increased by at least 5, 10, 20, 30, 40 or 50-fold, as compared to a reference sequence of SEQ ID NO: 138. In some embodiments, the brain region comprises a frontal cortex, sensory cortex, motor cortex, putamen, thalamus, cerebellar cortex, dentate nucleus, caudate, and/or hippocampus.

In some embodiments, an AAV capsid polypeptide, e.g., an AAV capsid variant, described herein delivers an increased level of a payload to a brain region. In some embodiments, the level of the payload is increased by at least 5, 10, 50, 100, 200, 500, 1,000, 2,000, 5,000, or 10,000-fold, as compared to a reference sequence of SEQ ID NO: 138. In some embodiments, the brain region comprises a frontal cortex, sensory cortex, motor cortex, putamen, thalamus, cerebellar cortex, dentate nucleus, caudate, and/or hippocampus.

In some embodiments, an AAV capsid polypeptide, e.g., an AAV capsid variant, described herein delivers an increased level of a payload to a spinal cord region. In some embodiments, the level of the payload is increased by at least 10, 20, 50, 100, 200, 300, 400, 500, 600, 700, 800 or 900-fold, as compared to a reference sequence of SEQ ID NO: 138. In some embodiments, the spinal cord region comprises a cervical, thoracic, and/or lumbar region.

In some embodiments, an AAV capsid polypeptide, e.g., an AAV capsid variant, described herein shows preferential transduction in a brain region relative to the transduction in the dorsal root ganglia (DRG).

In some embodiments, an AAV capsid polypeptide, e.g., an AAV capsid variant, described herein has an increased tropism for a muscle cell or tissue, e.g., a heart cell or tissue, relative to the tropism of a reference sequence comprising the amino acid sequence of SEQ ID NO: 138. In some embodiments, the AAV capsid variant delivers an increased level of a payload to a muscle region. In some embodiments, the payload is increased by at least 10, 15, 20, 30, or 40-fold, as compared to a reference sequence of SEQ ID NO: 138. In some embodiments, the muscle region comprises a heart muscle, quadriceps muscle, and/or a diaphragm muscle region. In some embodiments, the muscle region comprises a heart muscle region, e.g., a heart atrium muscle region or a heart ventricle muscle region.

In some embodiments, an AAV capsid polypeptide, e.g., an AAV capsid variant described herein results in greater than 1, 2, 5, 10, 20, 30, 40, 50, or 100 reads per sample, e.g., when analyzed by an NGS sequencing assay.

In some embodiments, an AAV capsid polypeptide, e.g., an AAV capsid variant, of the present disclosure has decreased tropism for the liver. In some embodiments, an AAV capsid variant comprises a modification, e.g., substitution (e.g., conservative substitution), insertion, or deletion, that results in reduced tropism (e.g., de-targeting) and/or activity in the liver. In some embodiments, the reduced tropism in the liver is compared to an otherwise similar capsid that does not comprise the modification, e.g., a wild-type capsid polypeptide. In some embodiments, an AAV capsid variant described comprises a modification, e.g., substitution (e.g., conservative substitution), insertion, or deletion, that results in one or more of the following properties: (1) reduced tropism in the liver; (2) de-targeted expression in the liver; (3) reduced activity in the liver; and/or (4) reduced binding to galactose. In some embodiments, the reduction in any one, or all of properties (1)-(3) is compared to an otherwise similar AAV capsid variant that does not comprise the modification. Exemplary modifications are provided in WO 2018/119330; Pulicherla et al. (2011) Mol. Ther. 19(6): 1070-1078; Adachi et al. (2014) Nature Communications 5(3075), DOI: 10.1038/ncomms4075; and Bell et al. (2012) J. Virol. 86(13): 7326-33; the contents of which are hereby incorporated by reference in their entirety. In some embodiments, the AAV capsid variant comprises a modification e.g., substitution (e.g., conservative substitution), insertion, or deletion, at position N470 (e.g., N470A), D271 (e.g., D271A), N272 (e.g., N297A), Y446 (e.g., Y446A), N498 (e.g., N498Y or N498I), W503 (e.g., W530R or W530A), L620 (e.g., L620F), or a combination thereof, relative to a reference sequence numbered according to SEQ ID NO: 138. In some embodiments, the AAV capsid variant comprises one, two, three, four, five or all of an amino acid other than N at position 470 (e.g., A), an amino acid other than D at position 271 (e.g., A), an amino acid other than N at position 272 (e.g., A), an amino acid other than Y at position 446 (e.g., A), and amino acid other than N at position 498/(e.g., Y or I), and amino acid other than W at position 503 (e.g., R or A), and amino acid other than L at position 620 (e.g., F), relative to a reference sequence numbered according to SEQ ID NO: 138. In some embodiments, the AAV capsid variant comprises a modification e.g., substitution (e.g., conservative substitution), insertion, or deletion, at position N470 (e.g., N470A), D271 (e.g., D271A), N272 (e.g., N297A), Y446 (e.g., Y446A), and W503 (e.g., W530R or W530A), relative to a reference sequence numbered according to SEQ ID NO: 138. In some embodiments, the AAV capsid variant comprises a modification e.g., substitution (e.g., conservative substitution), insertion, or deletion, at N498 (e.g., N498Y) and L620 (e.g., L620F).

In some embodiments, an AAV capsid variant comprised herein comprises a modification as described in Adachi et al. (2014) Nature Communications 5(3075), DOI: 10.1038/ncomms4075, the contents of which are hereby incorporated by reference in its entirety. Exemplary modifications that alter or do not alter tissue transduction in at least the brain, liver, heart, lung, and/or kidney can be found in Supplementary Data 2 showing the AAV Barcode-Seq data obtained with AAV9-AA-VBCLib of Adachi et al. (supra), the contents of which are hereby incorporated by reference in its entirety.

In some embodiments, an, AAV capsid polypeptide, e.g., an AAV capsid variant, of the present disclosure is isolated, e.g., recombinant. In some embodiments, a polynucleotide encoding an AAV capsid polypeptide, e.g., an AAV capsid variant, of the present disclosure is isolated, e.g., recombinant.

In some embodiments, the AAV capsid polypeptide, e.g., the AAV capsid variant, e.g., an AAV capsid variant described herein, comprises the amino acid sequence of any one of SEQ ID NOs: 3636-3647, or an amino acid sequence with at least 80% (e.g., at least about 85, 90, 95, 96, 97, 98, or 99%) sequence identity thereto. In some embodiments, the AAV capsid polypeptide, e.g., the AAV capsid variant, e.g., an AAV capsid variant described herein, comprises an amino acid sequence comprising at least one, two, or three modifications, substitutions (e.g., conservative substitutions), insertions, or deletions, but not more than 30, 20 or 10 modifications, substitutions (e.g., conservative substitutions), insertions, or deletions relative to the amino acid sequence of any one of SEQ ID NOs: 3636-3647. In some embodiments, the AAV capsid polypeptide, e.g., the AAV capsid variant, e.g., an AAV capsid variant described herein, comprises an amino acid sequence having at least one, two, or three but no more than 30, 20, or 10 different amino acids relative to the amino acid sequence of any one of SEQ ID NOs: 3636-3647.

In some embodiments, the AAV capsid polypeptide, e.g., the AAV capsid variant, e.g., an AAV capsid variant described herein, comprises the amino acid sequence of SEQ ID NO: 3636, or an amino acid sequence with at least 80% (e.g., at least about 85, 90, 95, 96, 97, 98, or 99%) sequence identity thereto. In some embodiments, the AAV capsid polypeptide, e.g., the AAV capsid variant, e.g., an AAV capsid variant described herein, comprises an amino acid sequence comprising at least one, two, or three modifications, substitutions (e.g., conservative substitutions), insertions, or deletions, but not more than 30, 20 or 10 modifications, substitutions (e.g., conservative substitutions), insertions, or deletions relative to the amino acid sequence of SEQ ID NO: 3636. In some embodiments, the AAV capsid polypeptide, e.g., the AAV capsid variant, e.g., an AAV capsid variant described herein, comprises an amino acid sequence having at least one, two, or three but no more than 30, 20, or 10 different amino acids relative to the amino acid sequence of SEQ ID NO: 3636.

In some embodiments, an, AAV capsid polypeptide, e.g., an AAV capsid variant, of the present disclosure is isolated, e.g., recombinant. In some embodiments, a polynucleotide encoding an AAV capsid polypeptide, e.g., an AAV capsid variant, of the present disclosure is isolated, e.g., recombinant.

The present disclosure refers to structural capsid proteins (including VP1, VP2 and VP3) which are encoded by capsid (Cap) genes. These capsid proteins form an outer protein structural shell (i.e. capsid) of a viral vector such as AAV. VP capsid proteins synthesized from Cap polynucleotides generally include a methionine as the first amino acid in the peptide sequence (Met1), which is associated with the start codon (AUG or ATG) in the corresponding Cap nucleotide sequence. However, it is common for a first-methionine (Met1) residue or generally any first amino acid (AA1) to be cleaved off after or during polypeptide synthesis by protein processing enzymes such as Met-aminopeptidases. This “Met/AA-clipping” process often correlates with a corresponding acetylation of the second amino acid in the polypeptide sequence (e.g., alanine, valine, serine, threonine, etc.). Met-clipping commonly occurs with VP1 and VP3 capsid proteins but can also occur with VP2 capsid proteins.

Where the Met/AA-clipping is incomplete, a mixture of one or more (one, two or three) VP capsid proteins comprising the viral capsid may be produced, some of which may include a Met1/AA1 amino acid (Met+/AA+) and some of which may lack a Met1/AA1 amino acid as a result of Met/AA-clipping (Met−/AA−). For further discussion regarding Met/AA-clipping in capsid proteins, see Jin, et al. Direct Liquid Chromatography/Mass Spectrometry Analysis for Complete Characterization of Recombinant Adeno-Associated Virus Capsid Proteins. Hum Gene Ther Methods. 2017 Oct. 28(5):255-267; Hwang, et al. N-Terminal Acetylation of Cellular Proteins Creates Specific Degradation Signals. Science. 2010 Feb. 19. 327(5968): 973-977; the contents of which are each incorporated herein by reference in their entirety.

According to the present disclosure, references to capsid proteins is not limited to either clipped (Met−/AA−) or unclipped (Met+/AA+) and may, in context, refer to independent capsid proteins, viral capsids comprised of a mixture of capsid proteins, and/or polynucleotide sequences (or fragments thereof) which encode, describe, produce or result in capsid proteins of the present disclosure. A direct reference to a “capsid protein” or “capsid polypeptide” (such as VP1, VP2 or VP2) may also comprise VP capsid proteins which include a Met1/AA1 amino acid (Met+/AA+) as well as corresponding VP capsid proteins which lack the Met1/AA1 amino acid as a result of Met/AA-clipping (Met−/AA−).

Further according to the present disclosure, a reference to a specific “SEQ ID NO:” (whether a protein or nucleic acid) which comprises or encodes, respectively, one or more capsid proteins which include a Met1/AA1 amino acid (Met+/AA+) should be understood to teach the VP capsid proteins which lack the Met1/AA1 amino acid as upon review of the sequence, it is readily apparent any sequence which merely lacks the first listed amino acid (whether or not Met1/AA1).

As a non-limiting example, reference to a VP1 polypeptide sequence which is 736 amino acids in length and which includes a “Met1” amino acid (Met+) encoded by the AUG/ATG start codon may also be understood to teach a VP1 polypeptide sequence which is 735 amino acids in length and which does not include the “Met1” amino acid (Met−) of the 736 amino acid Met+ sequence. As a second non-limiting example, reference to a VP1 polypeptide sequence which is 736 amino acids in length and which includes an “AA1” amino acid (AA1+) encoded by any NNN initiator codon may also be understood to teach a VP1 polypeptide sequence which is 735 amino acids in length and which does not include the “AA1” amino acid (AA1−) of the 736 amino acid AA1+ sequence.

References to viral capsids formed from VP capsid proteins (such as reference to specific AAV capsid serotypes), can incorporate VP capsid proteins which include a Met1/AA1 amino acid (Met+/AA1+), corresponding VP capsid proteins which lack the Met1/AA1 amino acid as a result of Met/AA1-clipping (Met−/AA1−), and combinations thereof (Met+/AA1+ and Met−/AA1−).

As a non-limiting example, an AAV capsid serotype can include VP1 (Met+/AA1+), VP1 (Met−/AA1−), or a combination of VP1 (Met+/AA1+) and VP1 (Met−/AA1−). An AAV capsid serotype can also include VP3 (Met+/AA1+), VP3 (Met−/AA1−), or a combination of VP3 (Met+/AA1+) and VP3 (Met−/AA1−); and can also include similar optional combinations of VP2 (Met+/AA1) and VP2 (Met−/AA1−).

Also provided herein are polynucleotide sequences encoding any of the AAV capsid variants described above and AAV particles, vectors, and cells comprising the same.

### Viral Genome of the AAV Particle

In some embodiments, an AAV particle as described herein comprising an AAV capsid polypeptide, e.g., AAV capsid variant, described herein, may be used for the delivery of a viral genome to a tissue (e.g., CNS, DRG, and/or muscle). In some embodiments, an AAV particle comprising an AAV capsid polypeptide, e.g., an AAV capsid variant, described herein can be used for delivery of a viral genome to a tissue or cell, e.g., CNS, DRG, or muscle cell or tissue. In some embodiments, an AAV particle of the present disclosure is a recombinant AAV particle. In some embodiments, an AAV particle of the present disclosure is an isolated AAV particle.

The viral genome may encode any payload, such as but not limited to a polypeptide (e.g., a therapeutic polypeptide), an antibody, an enzyme, an RNAi agent and/or components of a gene editing system. In one embodiment, the AAV particles described herein are used to deliver a payload to cells of the CNS, after intravenous delivery. In another embodiment, the AAV particles described herein are used to deliver a payload to cells of the DRG, after intravenous delivery. In some embodiments, the AAV particles described herein are used to deliver a payload to cells of a muscle, e.g., a heart muscle, after intravenous delivery.

In some embodiments, a viral genome of an AAV particle comprising an AAV capsid polypeptide, e.g., an AAV capsid variant, as described herein, comprises a nucleic acid comprising a transgene encoding a payload. In some embodiments, the viral genome comprises an inverted terminal repeat (ITR) sequence. In some embodiments, the viral genome comprises two ITR sequences, e.g., one at the 5′ end of the viral genome (e.g., 5′ relative to the encoded payload) and one at the 3′ end of the viral genome (e.g., 3′ relative to the encoded payload). In some embodiments, a viral genome of the AAV particles described herein (e.g., comprising an AAV capsid variant described herein) may comprise a regulatory element (e.g., promoter), untranslated regions (UTR), a miR binding site a polyadenylation sequence (polyA), a filler or stuffer sequence, an intron, and/or a linker sequence, e.g., for enhancing transgene expression.

In some embodiments, the viral genome components are selected and/or engineered for expression of a payload in a target tissue (e.g., a CNS tissue, a muscle tissue (e.g., heart), or DRG).

### Viral Genome Component: Inverted Terminal Repeats (ITRs)

In some embodiments, the AAV particle comprising an AAV capsid polypeptide, e.g., an AAV capsid variant, described herein comprises a viral genome comprising an ITR and a transgene encoding a payload. In some embodiment, the viral genome has two ITRs. In some embodiments, the two ITRs flank the nucleotide sequence encoding the payload at the 5′ and 3′ ends. In some embodiments, the ITRs function as origins of replication comprising recognition sites for replication. In some embodiments, the ITRs comprise sequence regions which can be complementary and symmetrically arranged. In some embodiments, the ITRs incorporated into viral genomes as described herein may be comprised of naturally occurring polynucleotide sequences or recombinantly derived polynucleotide sequences.

In some embodiments, the ITR may be of the same serotype (e.g., a serotype listed in Table 1) as the capsid polypeptide, e.g., capsid variant, selected from any of the known serotypes, or a variant thereof. In some embodiments, the ITR may be of a different serotype than the capsid. In one embodiment, the viral genome comprises two ITR sequence regions, wherein the ITRs are of the same serotype as one another. In another embodiment, the viral genome comprises two ITR sequence regions, wherein the ITRs are of different serotypes. Non-limiting examples include zero, one or both of the ITRs having the same serotype as the capsid. In one embodiment both ITRs of the viral genome of the AAV particle are AAV2 ITRs.

### Viral Genome Component: Promoters

In one embodiment, the payload region of the viral genome comprises at least one element to enhance the payload target specificity and expression (See e.g., Powell et al. Viral Expression Cassette Elements to Enhance Transgene Target Specificity and Expression in Gene Therapy, 2015; the contents of which are herein incorporated by reference in their entirety). Non-limiting examples of elements to enhance payload target specificity and expression include promoters, endogenous miRNAs, post-transcriptional regulatory elements (PREs), polyadenylation (PolyA) signal sequences and upstream enhancers (USEs), CMV enhancers and introns.

In some embodiments, an AAV particle comprising an AAV capsid variant described herein comprises a viral genome comprising a nucleic acid comprising a transgene encoding a payload, wherein the transgene is operably linked to a promoter. In some embodiments, the promoter is a species' specific promoter, an inducible promoter, tissue-specific, or cell cycle-specific (Parr et al., Nat. Med. 3:1145-9 (1997); the contents of which are herein incorporated by reference in their entirety).

In some embodiments the promoter may be naturally occurring or non-naturally occurring. Non-limiting examples of promoters include those from viruses, plants, mammals, or humans. In some embodiments, the promoters may be those from human cells or systems. In some embodiments, the promoter may be truncated or mutated, e.g., a promoter variant.

In some embodiments, the promoter is a ubiquitous promoter, e.g., capable of expression in multiple tissues. In some embodiments the promoter is a human elongation factor 1α-subunit (EF1α) promoter, the cytomegalovirus (CMV) immediate-early enhancer and/or promoter, the chicken β-actin (CBA) promoter and its derivative CAG, β glucuronidase (GUSB) promoter, or ubiquitin C (UBC) promoter. In some embodiments, the promoter is a cell or tissue specific promoter, e.g., capable of expression in tissues or cells of the central or peripheral nervous systems, regions within (e.g., frontal cortex), and/or sub-sets of cells therein (e.g., excitatory neurons). In some embodiments, the promoter is a cell-type specific promoter capable of expression a payload in excitatory neurons (e.g., glutamatergic), inhibitory neurons (e.g., GABA-ergic), neurons of the sympathetic or parasympathetic nervous system, sensory neurons, neurons of the dorsal root ganglia, motor neurons, or supportive cells of the nervous systems such as microglia, astrocytes, oligodendrocytes, and/or Schwann cells.

In some embodiments, the promoter is a liver promoter (e.g., hAAT, TBG), skeletal muscle specific promoter (e.g., desmin, MCK, C512), B cell promoter, monocyte promoter, leukocyte promoter, macrophage promoter, pancreatic acinar cell promoter, endothelial cell promoter, lung tissue promoter, and/or cardiac or cardiovascular promoter (e.g., αMHC, cTnT, and CMV-MLC2k).

In some embodiments, the promoter is a tissue-specific promoter for payload expression in a cell or tissue of the central nervous system. In some embodiments, the promoter is synapsin (Syn) promoter, glutamate vesicular transporter (VGLUT) promoter, vesicular GABA transporter (VGAT) promoter, parvalbumin (PV) promoter, sodium channel Naᵥ 1.8 promoter, tyrosine hydroxylase (TH) promoter, choline acetyltransferase (ChaT) promoter, methyl-CpG binding protein 2 (MeCP2) promoter, Ca²⁺/calmodulin-dependent protein kinase II (CaMKII) promoter, metabotropic glutamate receptor 2 (mGluR2) promoter, neurofilament light (NFL) or heavy (NFH) promoter, neuron-specific enolase (NSE) promoter, β-globin minigene nβ2 promoter, preproenkephalin (PPE) promoter, enkephalin (Enk) promoter, and excitatory amino acid transporter 2 (EAAT2) promoter. In some embodiments, the promoter is a cell-type specific promoter capable of expression in an astrocyte, e.g., a glial fibrillary acidic protein (GFAP) promoter and a EAAT2 promoter. In some embodiments, the promoter is a cell-type specific promoter capable of expression in an oligodendrocyte, e.g., a myelin basic protein (MBP) promoter.

In some embodiments, the promoter is a GFAP promoter. In some embodiments, the promoter is a synapsin (syn or syn1) promoter, or a fragment thereof.

In some embodiments, the promoter comprises an insulin promoter or a fragment thereof.

In some embodiments, the promoter of the viral genome described herein (e.g., comprised within an AAV particle comprising an AAV capsid variant described herein) comprises an EF-1α promoter or variant thereof, e.g., as provided in Table 6. In some embodiments, the EF-1α promoter comprises the nucleotide sequence of any one of SEQ ID NOs: 9000, 9001, 9003, 9004, 9008, 9009, 9011-9020, GCATG, CGTGAG, GT, GCTCCGGT, or GTAAG or any of the sequences provided in Table 6, a nucleotide sequence comprising at least one, two, or three but no more than four modifications, e.g., substitutions, relative to the nucleotide sequence of SEQ ID NOs: 9000-9020, or a nucleotide sequence with at least 70% (e.g., 80, 85%, 90%, 95%, 96%, 97%, 98%, or 99%) sequence identity to any one of SEQ ID NOs: 9000, 9001, 9003, 9004, 9008, 9009, 9011-9020, GCATG, CGTGAG, GT, GCTCCGGT, or GTAAG or any of the sequences provided in Table 6.

TABLE 6
Exemplary Promoter Variants

| | | SEQ ID |
|---|---|---|
| Description | Sequences | NO: |
| EF1a Promoter | CGTGAGGCTCCGGTGCCCGTCAGTGGGCAGAGCGCACATCGCCCACAGTCCCCG | 9000 |
| (intron | AGAAGTTGGGGGGAGGGGTCGGCAATTGAACCGGTGCCTAGAGAAGGTGGCGCG | |
| underlined) | GGGTAAACTGGGAAAGTGATGTCGTGTACTGGCTCCGCCTTTTTCCCGAGGGTG | |
| | GGGGAGAACCGTATATAAGTGCAGTAGTCGCCGTGAACGTTCTTTTTCGCAACG | |
| | GGTTTGCCGCCAGAACACAGGTAAGTGCCGTGTGTGGTTCCCGCGGGCCTGGCC | |
| | TCTTTACGGGTTATGGCCCTTGCGTGCCTTGAATTACTTCCACCTGGCTGCAGT | |
| | ACGTGATTCTTGATCCCGAGCTTCGGGTTGGAAGTGCGGGGGAGAGTTCGAGGC | |
| | CTTGCGCTTAAGGAGCCCCTTCGCCTCGTGCTTGAGTTGAGGCCTGGCCTGGGC | |
| | GCTGGGGCCGCCGCGTGCGAATCTGGTGGCACCTTCGCGCCTGTCTCGCTGCTT | |
| | TCGATAAGTCTCTAGCCATTTAAAATTTTTGATGACCTGCTGCGACGCTTTTTT | |
| | TCTGGCAAGATAGTCTTGTAAATGCGGGCCAAGATCTGCACACTGGTATTTCGG | |
| | TTTTTGGGGCCGCGGGCGGCGACGGGGCCCGTGCGTCCCAGCGCACATGTTCGG | |
| | CGAGGCGGGGCCTGCGAGCGCGGCCACCGAGAATCGGACGGGGGTAGTCTCAAG | |
| | CTGGCCGGCCTGCTCTGGTGCCTGGCCTCGCGCCGCCGTGTATCGCCCCGCCCT | |
| | GGGCGGCAAGGCTGGCCCGGTCGGCACCAGTTGCGTGAGCGGAAAGATGGCCGC | |
| | TTCCCGGCCCTGCTGCAGGGAGCTCAAAATGGAGGACGCGGCGCTCGGGAGAGC | |
| | GGGCGGGTGAGTCACCCACACAAAGGAAAAGGGCCTTTCCGTCCTCAGCCGTCG | |
| | CTTCATGTGACTCCACGGAGTACCGGGCGCCGTCCAGGCACCTCGATTAGTTCT | |
| | CGAGCTTTTGGAGTACGTCGTCTTTAGGTTGGGGGGAGGGGTTTTATGCGATGG | |
| | AGTTTCCCCACACTGAGTGGGTGGAGACTGAAGTTAGGCCAGCTTGGCACTTGA | |
| | TGTAATTCTCCTTGGAATTTGCCCTTTTTGAGTTTGGATCTTGGTTCATTCTCA | |
| | AGCCTCAGACAGTGGTTCAAAGTTTTTTTCTTCCATTTCAGGTGTCGTGA | |
| miniEF1a | GCCCGTCAGTGGGCAGAGCGCACATCGCCCACAGTCCCCGAGAAGTTGGGGGGA | 9001 |
| | GGGGTCGGCAATTGAACCGGTGCCTAGAGAAGGTGGCGCGGGGTAAACTGGGAA | |
| | AGTGATGTCGTGTACTGGCTCCGCCTTTTTCCCGAGGGTGGGGGAGAACCGTAT | |
| | ATAAGTGCAGTAGTCGCCGTGAACGTTCTTTTTCGCAACGGGTTTGCCGCCAGA | |
| | ACACGCGTAAG | |
| Promoter | GCATG | |
| Variant 1 | | |
| Promoter | GGTGGAGAAGAGCATG | 9003 |
| Variant 2 | | |
| Promoter | GTCATCACTGAGGTGGAGAAGAGCATG | 9004 |
| Variant 3 | | |
| Promoter | CGTGAG | |
| Variant 4 | | |
| Promoter | GT | |
| Variant 5 | | |
| Promoter | GCTCCGGT | |
| Variant | | |
| 6 | | |
| Promoter | GCCCGTCAGTGGGCAGAGCGCACATCGCCCACAGTCCCCGAGAAGTTGGGGGGA | 9008 |
| Variant 19 | GGGGTCGGCAATTGAACCGGTGCCTAGAGAAGGTGGCGCGGGGTAAACTGGGAA | |
| | AGTGATGTCGTGTACTGGCTCCGCCTTTTTCCCGAGGGTGGGGGAGAACCGTAT | |
| | ATAAGTGCAGTAGTCGCCGTGAACGTTCTTTTTCGCAACGGGTTTGCCGCCAGA | |
| | ACACAG | |
| Promoter | GCCCGTCAGTGGGCAGAGCGCACATCGCCCACAGTCCCCGAGAAGTTGGGGGGA | 9009 |
| Variant | GGGGTCGGCAATTGAACCGGTGCCTAGAGAAGGTGGCGCGGGGTAAACTGGGAA | |
| 20 | AGTGATGTCGTGTACTGGCTCCGCCTTTTTCCCGAGGGTGGGGGAGAACCGTAT | |
| | ATAAGTGCAGTAGTCGCCGTGAACGTTCTTTTTCGCAACGGGTTTGCCGCCAGA | |
| | ACACGC | |
| Promoter | GTAAG | |
| Variant 7 | | |
| Promoter | GTGCCCGTCAGTGGGCAGAGCGCACATCGCCCACAGTCCCCGAGAAGTTGGGGG | 9011 |
| Variant 8 | GAGGGGTCGGCAATTGAACCGGTGCCTAGAGAAGGTGGCGCGGGGTAAACTGGG | |
| | AAAGTGATGTCGTGTACTGGCTCCGCCTTTTTCCCGAGGGTGGGGGAGAACCGT | |
| | ATATAAGTGCAGTAGTCGCCGTGAACGTTCTTTTTCGCAACGGGTTTGCCGCCA | |
| | GAACACGCGTAAG | |
| Promoter | GCTCCGGTGCCCGTCAGTGGGCAGAGCGCACATCGCCCACAGTCCCCGAGAAGT | 9012 |
| Variant 9 | TGGGGGGAGGGGTCGGCAATTGAACCGGTGCCTAGAGAAGGTGGCGCGGGGTAA | |
| | ACTGGGAAAGTGATGTCGTGTACTGGCTCCGCCTTTTTCCCGAGGGGGGGGAG | |
| | AACCGTATATAAGTGCAGTAGTCGCCGTGAACGTTCTTTTTCGCAACGGGTTTG | |
| | CCGCCAGAACACGCGTAAG | |
| Promoter | CGTGAGGCTCCGGTGCCCGTCAGTGGGCAGAGCGCACATCGCCCACAGTCCCCG | 9013 |
| Variant 10 | AGAAGTTGGGGGGAGGGGTCGGCAATTGAACCGGTGCCTAGAGAAGGTGGCGCG | |
| | GGGTAAACTGGGAAAGTGATGTCGTGTACTGGCTCCGCCTTTTTCCCGAGGGTG | |
| | GGGGAGAACCGTATATAAGTGCAGTAGTCGCCGTGAACGTTCTTTTTCGCAACG | |
| | GGTTTGCCGCCAGAACACGCGTAAG | |
| Promoter | CGTGAGGCTCCGGTGCCCGTCAGTGGGCAGAGCGCACATCGCCCACAGTCCCCG | 9014 |
| Variant 11 | AGAAGTTGGGGGGAGGGGTCGGCAATTGAACCGGTGCCTAGAGAAGGTGGCGCG | |
| | GGGTAAACTGGGAAAGTGATGTCGTGTACTGGCTCCGCCTTTTTCCCGAGGGTG | |
| | GGGGAGAACCGTATATAAGTGCAGTAGTCGCCGTGAACGTTCTTTTTCGCAACG | |
| | GGTTTGCCGCCAGAACACAG | |
| Promoter | GCATGCGTGAGGCTCCGGTGCCCGTCAGTGGGCAGAGCGCACATCGCCCACAGT | 9015 |
| Variant 12 | CCCCGAGAAGTTGGGGGGAGGGGTCGGCAATTGAACCGGTGCCTAGAGAAGGTG | |
| | GCGCGGGGTAAACTGGGAAAGTGATGTCGTGTACTGGCTCCGCCTTTTTCCCGA | |
| | GGGTGGGGGAGAACCGTATATAAGTGCAGTAGTCGCCGTGAACGTTCTTTTTCG | |
| | CAACGGGTTTGCCGCCAGAACACGCGTAAG | |
| Promoter | GCATGCGTGAGGCTCCGGTGCCCGTCAGTGGGCAGAGCGCACATCGCCCACAGT | 9016 |
| Variant 13 | CCCCGAGAAGTTGGGGGGAGGGGTCGGCAATTGAACCGGTGCCTAGAGAAGGTG | |
| | GCGCGGGGTAAACTGGGAAAGTGATGTCGTGTACTGGCTCCGCCTTTTTCCCGA | |
| | GGGTGGGGGAGAACCGTATATAAGTGCAGTAGTCGCCGTGAACGTTCTTTTTCG | |
| | CAACGGGTTTGCCGCCAGAACACAG | |
| Promoter | GGTGGAGAAGAGCATGCGTGAGGCTCCGGTGCCCGTCAGTGGGCAGAGCGCACA | 9017 |
| Variant 14 | TCGCCCACAGTCCCCGAGAAGITGGGGGGAGGGGTCGGCAATTGAACCGGTGCC | |
| | TAGAGAAGGTGGCGCGGGGTAAACTGGGAAAGTGATGTCGTGTACTGGCTCCGC | |
| | CTTTTTCCCGAGGGGGGGGAGAACCGTATATAAGTGCAGTAGTCGCCGTGAAC | |
| | GTTCTTTTTCGCAACGGGTTTGCCGCCAGAACACGCGTAAG | |
| Promoter | GGTGGAGAAGAGCATGCGTGAGGCTCCGGTGCCCGTCAGTGGGCAGAGCGCACA | 9018 |
| Variant 15 | TCGCCCACAGTCCCCGAGAAGITGGGGGGAGGGGTCGGCAATTGAACCGGTGCC | |
| | TAGAGAAGGTGGCGCGGGGTAAACTGGGAAAGTGATGTCGTGTACTGGCTCCGC | |
| | CTTTTTCCCGAGGGTGGGGGAGAACCGTATATAAGTGCAGTAGTCGCCGTGAAC | |
| | GTTCTTTTTCGCAACGGGTTTGCCGCCAGAACACAG | |
| Promoter | GTCATCACTGAGGIGGAGAAGAGCATGCGTGAGGCTCCGGTGCCCGTCAGTGGG | 9019 |
| Variant 16 | CAGAGCGCACATCGCCCACAGTCCCCGAGAAGTTGGGGGGAGGGGTCGGCAATT | |
| | GAACCGGTGCCTAGAGAAGGTGGCGCGGGGTAAACTGGGAAAGTGATGTCGTGT | |
| | ACTGGCTCCGCCTTTTTCCCGAGGGTGGGGGAGAACCGTATATAAGTGCAGTAG | |
| | TCGCCGTGAACGTTCTTTTTCGCAACGGGTTTGCCGCCAGAACACGCGTAAG | |
| Promoter | GTCATCACTGAGGTGGAGAAGAGCATGCGTGAGGCTCCGGTGCCCGTCAGTGGG | 9020 |
| Variant 18 | CAGAGCGCACATCGCCCACAGTCCCCGAGAAGTTGGGGGGAGGGGTCGGCAATT | |
| | GAACCGGTGCCTAGAGAAGGTGGCGCGGGGTAAACTGGGAAAGTGATGTCGTGT | |
| | ACTGGCTCCGCCTTTTTCCCGAGGGGGGGGAGAACCGTATATAAGTGCAGTAG | |
| | TCGCCGTGAACGTTCTTTTTCGCAACGGGTTTGCCGCCAGAACACAG | |

### Viral Genome Component: Untranslated Regions (UTRs)

In some embodiments, wild type untranslated regions (UTRs) of a gene are transcribed but not translated. Generally, the 5′ UTR starts at the transcription start site and ends at the start codon and the 3′ UTR starts immediately following the stop codon and continues until the termination signal for transcription.

Features typically found in abundantly expressed genes of specific target organs (e.g., CNS tissue, muscle, or DRG) may be engineered into UTRs to enhance stability and protein production. As a non-limiting example, a 5′ UTR from mRNA normally expressed in the brain (e.g., huntingtin) may be used in the viral genomes of the AAV particles described herein to enhance expression in neuronal cells or other cells of the central nervous system.

While not wishing to be bound by theory, wild-type 5′ untranslated regions (UTRs) include features which play roles in translation initiation. Kozak sequences, which are commonly known to be involved in the process by which the ribosome initiates translation of many genes, are usually included in 5′ UTRs. Kozak sequences have the consensus CCR(A/G)CCAUGG, where R is a purine (adenine or guanine) three bases upstream of the start codon (ATG), which is followed by another ‘G’.

In one embodiment, the 5′UTR in the viral genome includes a Kozak sequence.

In one embodiment, the 5′UTR in the viral genome does not include a Kozak sequence.

While not wishing to be bound by theory, wild-type 3′ UTRs are known to have stretches of Adenosines and Uridines embedded therein. These AU rich signatures are particularly prevalent in genes with high rates of turnover. Based on their sequence features and functional properties, the AU rich elements (AREs) can be separated into three classes (Chen et al, 1995, the contents of which are herein incorporated by reference in its entirety): Class I AREs, such as, but not limited to, c-Myc and MyoD, contain several dispersed copies of an AUUUA motif within U-rich regions. Class II AREs, such as, but not limited to, GM-CSF and TNF-a, possess two or more overlapping UUAUUUA(U/A)(U/A) nonamers. Class III ARES, such as, but not limited to, c-Jun and Myogenin, are less well defined. These U rich regions do not contain an AUUUA motif. Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into the 3′ UTR of nucleic acid molecules will lead to HuR binding and thus, stabilization of the message in vivo.

Introduction, removal or modification of 3′ UTR AU rich elements (AREs) can be used to modulate the stability of a polynucleotide. When engineering specific polynucleotides, e.g., payload regions of viral genomes, one or more copies of an ARE can be introduced to make polynucleotides less stable and thereby curtail translation and decrease production of the resultant protein. Likewise, AREs can be identified and removed or mutated to increase the intracellular stability and thus increase translation and production of the resultant protein.

In one embodiment, the 3′ UTR of the viral genome may include an oligo(dT) sequence for templated addition of a poly-A tail.

In one embodiment, the viral genome may include at least one miRNA seed, binding site or full sequence. microRNAs (or miRNA or miR) are 19-25 nucleotide noncoding RNAs that bind to the sites of nucleic acid targets and down-regulate gene expression either by reducing nucleic acid molecule stability or by inhibiting translation. In some embodiments, a microRNA sequence comprises a seed region, e.g., a sequence in the region of positions 2-8 of the mature microRNA, which has Watson-Crick sequence fully or partially complementarity to the miRNA target sequence of the nucleic acid.

In one embodiment, the viral genome may be engineered to include, alter or remove at least one miRNA binding site, full sequence or seed region.

Any UTR from any gene known in the art may be incorporated into the viral genome of the AAV particle. These UTRs, or portions thereof, may be placed in the same orientation as in the gene from which they were selected, or they may be altered in orientation or location. In one embodiment, the UTR used in the viral genome of the AAV particle may be inverted, shortened, lengthened, made with one or more other 5′ UTRs or 3′ UTRs known in the art. As used herein, the term “altered” as it relates to a UTR, means that the UTR has been changed in some way in relation to a reference sequence. For example, a 3′ or 5′ UTR may be altered relative to a wild type or native UTR by the change in orientation or location as taught above or may be altered by the inclusion of additional nucleotides, deletion of nucleotides, swapping or transposition of nucleotides.

In one embodiment, the viral genome of the AAV particle comprises at least one artificial UTR which is not a variant of a wild type UTR.

In one embodiment, the viral genome of the AAV particle comprises UTRs which have been selected from a family of transcripts whose proteins share a common function, structure, feature or property.

### Viral Genome Component: Polyadenylation Sequence

The viral genome of the AAV particle described herein (e.g., an AAV particle comprising an AAV capsid polypeptide, e.g., an AAV capsid variant, described herein) may comprise a polyadenylation sequence. In some embodiments, the viral genome of the AAV particle (e.g., an AAV particle comprising an AAV capsid polypeptide, e.g., an AAV capsid variant, described herein) comprises a polyadenylation sequence between the 3′ end of the nucleotide sequence encoding the payload and the 5′ end of the 3′ITR.

### Viral Genome Component: Introns

In some embodiments, the viral genome of the AAV particle as described herein (e.g., an AAV particle comprising an AAV capsid polypeptide, e.g., an AAV capsid variant), comprises an element to enhance the payload target specificity and expression (See e.g., Powell et al. Viral Expression Cassette Elements to Enhance Transgene Target Specificity and Expression in Gene Therapy, Discov. Med, 2015, 19(102): 49-57; the contents of which are herein incorporated by reference in their entirety) such as an intron. Non-limiting examples of introns include, MVM (67-97 bps), F.IX truncated intron 1 (300 bps), β-globin SD/immunoglobulin heavy chain splice acceptor (250 bps), adenovirus splice donor/immunoglobin splice acceptor (500 bps), SV40 late splice donor/splice acceptor (19S/16S) (180 bps) and hybrid adenovirus splice donor/IgG splice acceptor (230 bps).

### Viral Genome Component: Stuffer Sequences

In some embodiments, the viral genome of an AAV particle described herein (e.g., an AAV particle comprising an AAV capsid polypeptide, e.g., an AAV capsid variant), comprises an element to improve packaging efficiency and expression, such as a stuffer or filler sequence. Non-limiting examples of stuffer sequences include albumin and/or alpha-1 antitrypsin. Any known viral, mammalian, or plant sequence may be manipulated for use as a stuffer sequence.

In one embodiment, the stuffer or filler sequence may be from about 100-3500 nucleotides in length. The stuffer sequence may have a length of about 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900 or 3000 nucleotides.

Viral Genome Component: miRNA

In some embodiments, the viral genome comprises a sequence encoding a miRNA to reduce the expression of the payload in a tissue or cell, e.g., the DRG (dorsal root ganglion), or neurons of other ganglia, such as those of the sympathetic or parasympathetic nervous system. In some embodiments, a miRNA, e.g., a miR183, a miR182, and/or miR96, may be encoded in the viral genome to modulate, e.g., reduce the expression, of the viral genome in a DRG neuron. As another non-limiting example, a miR-122 miRNA may be encoded in the viral genome to modulate, e.g., reduce, the expression of the viral genome in the liver. In some embodiments, a miRNA, e.g., a miR-142-3p, may be encoded in the viral genome to modulate, e.g., reduce, the expression, of the viral genome in a cell or tissue of the hematopoietic lineage, including for example immune cells (e.g., antigen presenting cells or APC, including dendritic cells (DCs), macrophages, and B-lymphocytes). In some embodiments, a miRNA, e.g., a miR-1, may be encoded in the viral genome to modulate, e.g., reduce, the expression, of the viral genome in a cell or tissue of the heart.

### Viral Genome Component: miR Binding Site

Tissue- or cell-specific expression of the AAV viral particles disclosed herein can be enhanced by introducing tissue- or cell-specific regulatory sequences, e.g., promoters, enhancers, microRNA binding sites, e.g., a detargeting site. Without wishing to be bound by theory, it is believed that an encoded miR binding site can modulate, e.g., prevent, suppress, or otherwise inhibit, the expression of a gene of interest on the viral genome disclosed herein, based on the expression of the corresponding endogenous microRNA (miRNA) or a corresponding controlled exogenous miRNA in a tissue or cell, e.g., a non-targeting cell or tissue. In some embodiments, a miR binding site modulates, e.g., reduces, expression of the payload encoded by a viral genome of an AAV particle described herein in a cell or tissue where the corresponding mRNA is expressed.

In some embodiments, the viral genome of an AAV particle described herein comprises a nucleotide sequence encoding a microRNA binding site, e.g., a detargeting site. In some embodiments, the viral genome of an AAV particle described herein comprises a nucleotide sequence encoding a miR binding site, a microRNA binding site series (miR BSs), or a reverse complement thereof.

In some embodiments, the nucleotide sequence encoding the miR binding site series or the miR binding site is located in the 3′-UTR region of the viral genome (e.g., 3′ relative to the nucleotide sequence encoding a payload), e.g., before the polyA sequence, 5′-UTR region of the viral genome (e.g., 5′ relative to the nucleotide sequence encoding a payload), or both.

In some embodiments, the encoded miR binding site series comprise at least 1-5 copies, e.g., at least 1-3, 2-4, 3-5, 1, 2, 3, 4, 5 or more copies of a miR binding site (miR BS). In some embodiments, all copies are identical, e.g., comprise the same miR binding site. In some embodiments, the miR binding sites within the encoded miR binding site series are continuous and not separated by a spacer. In some embodiments, the miR binding sites within an encoded miR binding site series are separated by a spacer, e.g., a non-coding sequence. In some embodiments, the spacer is about 1 to 6 nucleotides or about 5 to 10 nucleotides, e.g., about 7-8 nucleotides, nucleotides in length. In some embodiments, the spacer coding sequence or reverse complement thereof comprises one or more of (i) GGAT; (ii) CACGTG; (iii) GCATGC, or a repeat of one or more of (i)-(iii). In some embodiments, the spacer comprises the nucleotide sequence of GATAGTTA, or a nucleotide sequence having at least one, two, or three modifications, e.g., substitutions (e.g., conservative substitutions), insertions, or deletions, but no more than four modifications, e.g., substitutions (e.g., conservative substitutions), insertions, or deletions, of GATAGTTA.

In some embodiments, the encoded miR binding site series comprise at least 1-5 copies, e.g., at least 1-3, 2-4, 3-5, 1, 2, 3, 4, 5 or more copies of a miR binding site (miR BS). In some embodiments, at least 1, 2, 3, 4, 5, or all of the copies are different, e.g., comprise a different miR binding site. In some embodiments, the miR binding sites within the encoded miR binding site series are continuous and not separated by a spacer. In some embodiments, the miR binding sites within an encoded miR binding site series are separated by a spacer, e.g., a non-coding sequence. In some embodiments, the spacer is about 1 to 6 nucleotides or about 5 to 10 nucleotides, e.g., about 7-8 nucleotides, in length. In some embodiments, the spacer comprises one or more of (i) GGAT; (ii) CACGTG; (iii) GCATGC, or a repeat of one or more of (i)-(iii). In some embodiments, the spacer comprises the nucleotide sequence of GATAGTTA, or a nucleotide sequence having at least one, two, or three modifications, e.g., substitutions (e.g., conservative substitutions), insertions, or deletions, but no more than four modifications, e.g., substitutions (e.g., conservative substitutions), insertions, or deletions, of GATAGTTA.

In some embodiments, the encoded miR binding site is substantially identical (e.g., at least 70%, 75%, 80%, 85%, 90%, 95%, 99% or 100% identical), to the miR in the host cell. In some embodiments, the encoded miR binding site comprises at least 1, 2, 3, 4, or 5 mismatches or no more than 6, 7, 8, 9, or 10 mismatches to a miR in the host cell. In some embodiments, the mismatched nucleotides are contiguous. In some embodiments, the mismatched nucleotides are non-contiguous. In some embodiments, the mismatched nucleotides occur outside the seed region-binding sequence of the miR binding site, such as at one or both ends of the miR binding site. In some embodiments, the miR binding site is 100% identical to the miR in the host cell.

In some embodiments, the nucleotide sequence encoding the miR binding site is substantially complimentary (e.g., at least 70%, 75%, 80%, 85%, 90%, 95%, 99% or 100% complimentary), to the miR in the host cell. In some embodiments, to complementary sequence of the nucleotide sequence encoding the miR binding site comprises at least 1, 2, 3, 4, or 5 mismatches or no more than 6, 7, 8, 9, or 10 mismatches to a miR in the host cell. In some embodiments, the mismatched nucleotides are contiguous. In some embodiments, the mismatched nucleotides are non-contiguous. In some embodiments, the mismatched nucleotides occur outside the seed region-binding sequence of the miR binding site, such as at one or both ends of the miR binding site. In some embodiments, the encoded miR binding site is 100% complimentary to the miR in the host cell.

In some embodiments, an encoded miR binding site or sequence region is at least about 10 to about 125 nucleotides in length, e.g., at least about 10 to 50 nucleotides, 10 to 100 nucleotides, 50 to 100 nucleotides, 50 to 125 nucleotides, or 100 to 125 nucleotides in length. In some embodiments, an encoded miR binding site or sequence region is at least about 7 to about 28 nucleotides in length, e.g., at least about 8-28 nucleotides, 7-28 nucleotides, 8-18 nucleotides, 12-28 nucleotides, 20-26 nucleotides, 22 nucleotides, 24 nucleotides, or 26 nucleotides in length, and optionally comprises at least one consecutive region (e.g., 7 or 8 nucleotides) complementary (e.g., fully or partially complementary) to the seed sequence of a miRNA (e.g., a miR122, a miR142, a miR183, or a miR1).

In some embodiments, the encoded miR binding site is complementary (e.g., fully or partially complementary) to a miR expressed in liver or hepatocytes, such as miR122. In some embodiments, the encoded miR binding site or encoded miR binding site series comprises a miR122 binding site sequence. In some embodiments, the encoded miR122 binding site comprises the nucleotide sequence of ACAAACACCATTGTCACACTCCA (SEQ ID NO: 3672), or a nucleotide sequence having at least 50%, 55%, 60%, 65%, 70%, 75%, 80%, 85%, 90%, at least 95%, at least 99%, or 100% sequence identity, or having at least one, two, three, four, five, six, or seven modifications, e.g., substitutions (e.g., conservative substitutions), insertions, or deletions, but no more than ten modifications, e.g., substitutions (e.g., conservative substitutions), insertions, or deletions, to SEQ ID NO: 3672, e.g., wherein the modification can result in a mismatch between the encoded miR binding site and the corresponding miRNA. In some embodiments, the viral genome comprises at least 2, 3, 4, or 5 copies of the encoded miR122 binding site, e.g., an encoded miR122 binding site series, optionally wherein the encoded miR122 binding site series comprises the nucleotide sequence of: ACAAACACCATTGTCACACTCCACACAAACACCATTGTCACACTCCACACAAACACCATTGT CACACTCCA (SEQ ID NO: 3673), or a nucleotide sequence having at least 50%, 55%, 60%, 65%, 70%, 75%, 80%, 85%, 90%, at least 95%, at least 99%, or 100% sequence identity, or having at least one, two, three, four, five, six, or seven modifications, e.g., substitutions (e.g., conservative substitutions), insertions, or deletions, but no more than ten modifications, e.g., substitutions (e.g., conservative substitutions), insertions, or deletions, to SEQ ID NO: 3673, e.g., wherein the modification can result in a mismatch between the encoded miR binding site and the corresponding miRNA. In some embodiments, at least two of the encoded miR122 binding sites are connected directly, e.g., without a spacer. In other embodiments, at least two of the encoded miR122 binding sites are separated by a spacer, e.g., 1, 2, 3, 4, 5, 6, 7, 8, 9, or 10 nucleotides in length, which is located between two or more consecutive encoded miR122 binding site sequences. In embodiments, the spacer is about 1 to 6 nucleotides or about 5 to 10 nucleotides, e.g., about 7-8, in length. In some embodiments, the spacer coding sequence or reverse complement thereof comprises one or more of (i) GGAT; (ii) CACGTG; (iii) GCATGC, or a repeat of one or more of (i)-(iii). In some embodiments, an encoded miR binding site series comprises at least 3-5 copies (e.g., 4 copies) of a miR122 binding site, with or without a spacer, wherein the spacer is about 1 to 6 nucleotides or about 5 to 10 nucleotides, e.g., about 7-8 nucleotides or about 8 nucleotides, in length. In some embodiments, the spacer comprises the nucleotide sequence of GATAGTTA, or a nucleotide sequence having at least one, two, or three modifications, e.g., substitutions (e.g., conservative substitutions), insertions, or deletions, but no more than four modifications, e.g., substitutions (e.g., conservative substitutions), insertions, or deletions, of GATAGTTA.

In some embodiments, the encoded miR binding site is complementary (e.g., fully or partially complementary) to a miR expressed in the heart. In embodiments, the encoded miR binding site or encoded miR binding site series comprises a miR-1 binding site. In some embodiments, the encoded miR-1 binding site comprises the nucleotide sequence of ATACATACTTCTTTACATTCCA (SEQ ID NO: 4679), a nucleotide sequence having at least 50%, 55%, 60%, 65%, 70%, 75%, 80%, 85%, 90%, at least 95%, at least 99%, or 100% sequence identity, or having at least one, two, three, four, five, six, or seven modifications e.g., substitutions (e.g., conservative substitutions), insertions, or deletions, but no more than ten modifications, e.g., substitutions (e.g., conservative substitutions), insertions, or deletions, to SEQ ID NO: 4679, e.g., wherein the modification can result in a mismatch between the encoded miR binding site and the corresponding miRNA. In some embodiments, the viral genome comprises at least 2, 3, 4, or 5 copies of the encoded miR-1 binding site, e.g., an encoded miR-1 binding site series. In some embodiments, the at least 2, 3, 4, or 5 copies (e.g., 2 or 3 copies) of the encoded miR-1 binding site are continuous (e.g., not separated by a spacer) or separated by a spacer. In some embodiments, the spacer is about 1 to 6 nucleotides or about 5 to 10 nucleotides, e.g., about 7-8 nucleotides or about 8 nucleotides, in length. In some embodiments, the spacer sequence comprises one or more of (i) GGAT; (ii) CACGTG; (iii) GCATGC, or a repeat of one or more of (i)-(iii). In some embodiments, the spacer comprises the nucleotide sequence of GATAGTTA, or a nucleotide sequence having at least one, two, or three modifications, e.g., substitutions (e.g., conservative substitutions), insertions, or deletions, but no more than four modifications, e.g., substitutions (e.g., conservative substitutions), insertions, or deletions, of GATAGTTA.

In some embodiments, the encoded miR binding site is complementary (e.g., fully or partially complementary) to a miR expressed in hematopoietic lineage, including immune cells (e.g., antigen presenting cells or APC, including dendritic cells (DCs), macrophages, and B-lymphocytes). In some embodiments, the encoded miR binding site complementary to a miR expressed in hematopoietic lineage comprises a nucleotide sequence disclosed, e.g., in US 2018/0066279, the contents of which are incorporated by reference herein in its entirety.

In embodiments, the encoded miR binding site or encoded miR binding site series comprises a miR-142-3p binding site sequence. In some embodiments, the encoded miR-142-3p binding site comprises the nucleotide sequence of TCCATAAAGTAGGAAACACTACA (SEQ ID NO: 3674), a nucleotide sequence having at least 50%, 55%, 60%, 65%, 70%, 75%, 80%, 85%, 90%, at least 95%, at least 99%, or 100% sequence identity, or having at least one, two, three, four, five, six, or seven modifications e.g., substitutions (e.g., conservative substitutions), insertions, or deletions, but no more than ten modifications, e.g., substitutions (e.g., conservative substitutions), insertions, or deletions, to SEQ ID NO: 3674, e.g., wherein the modification can result in a mismatch between the encoded miR binding site and the corresponding miRNA. In some embodiments, the viral genome comprises at least 2, 3, 4, or 5 copies of the encoded miR-142-3p binding site, e.g., an encoded miR-142-3p binding site series. In some embodiments, the at least 2, 3, 4, or 5 copies (e.g., 2 or 3 copies) of the encoded miR-142-3p binding site are continuous (e.g., not separated by a spacer) or separated by a spacer. In some embodiments, the spacer is about 1 to 6 nucleotides or about 5 to 10 nucleotides, e.g., about 7-8 nucleotides or about 8 nucleotides, in length. In some embodiments, the spacer sequence comprises one or more of (i) GGAT; (ii) CACGTG; (iii) GCATGC, or a repeat of one or more of (i)-(iii). In some embodiments, the spacer comprises the nucleotide sequence of, or a nucleotide sequence having at least one, two, or three modifications, e.g., substitutions (e.g., conservative substitutions), insertions, or deletions, but no more than four modifications, e.g., substitutions (e.g., conservative substitutions), insertions, or deletions, of GATAGTTA.

In some embodiments, the encoded miR binding site is complementary (e.g., fully complementary or partially complementary) to a miR expressed in a DRG (dorsal root ganglion) neuron, e.g., a miR183, a miR182, and/or miR96 binding site. In some embodiments, the encoded miR binding site is complementary to a miR expressed in expressed in a DRG neuron comprises a nucleotide sequence disclosed, e.g., in WO2020/132455, the contents of which are incorporated by reference herein in its entirety.

In some embodiments, the encoded miR binding site or encoded miR binding site series comprises a miR183 binding site sequence. In some embodiments, the encoded miR183 binding site comprises the nucleotide sequence of AGTGAATTCTACCAGTGCCATA (SEQ ID NO: 3675), or a nucleotide sequence having at least 50%, 55%, 60%, 65%, 70%, 75%, 80%, 85%, 90%, at least 95%, at least 99%, or 100% sequence identity, or having at least one, two, three, four, five, six, or seven modifications, e.g., substitutions (e.g., conservative substitutions), insertions, or deletions, but no more than ten modifications, e.g., substitutions (e.g., conservative substitutions), insertions, or deletions, to SEQ ID NO: 3675, e.g., wherein the modification can result in a mismatch between the encoded miR binding site and the corresponding miRNA. In some embodiments, the sequence complementary to the seed sequence corresponds to the double underlined of the encoded miR-183 binding site sequence. In some embodiments, the viral genome comprises at least comprises at least 2, 3, 4, or 5 copies (e.g., at least 2 or 3 copies) of the encoded miR183 binding site, e.g., an encoded miR183 binding site. In some embodiments, the at least 2, 3, 4, or 5 copies (e.g., 2 or 3 copies) of the encoded miR183 binding site are continuous (e.g., not separated by a spacer) or separated by a spacer. In some embodiments, the spacer is about 1 to 6 nucleotides or about 5 to 10 nucleotides, e.g., about 7-8 nucleotides or about 8 nucleotides, in length. In some embodiments, the spacer comprises the nucleotide sequence of GATAGTTA, or a nucleotide sequence having at least one, two, or three modifications, e.g., substitutions (e.g., conservative substitutions), insertions, or deletions, but no more than four modifications, e.g., substitutions (e.g., conservative substitutions), insertions, or deletions, of GATAGTTA. In some embodiments, the spacer sequence comprises one or more of (i) GGAT; (ii) CACGTG; (iii) GCATGC, or a repeat of one or more of (i)-(iii).

In some embodiments, the encoded miR binding site or the encoded miR binding site series comprises a miR182 binding site sequence. In some embodiments, the encoded miR182 binding site comprises, the nucleotide sequence of AGTGTGAGTTCTACCATTGCCAAA (SEQ ID NO: 3676), a sequence having at least 50%, 55%, 60%, 65%, 70%, 75%, 80%, 85%, 90%, at least 95%, at least 99%, or 100% sequence identity, or having at least one, two, three, four, five, six, or seven modifications, e.g., substitutions (e.g., conservative substitutions), insertions, or deletions, but no more than ten modifications, e.g., substitutions (e.g., conservative substitutions), insertions, or deletions, to SEQ ID NO: 3676, e.g., wherein the modification can result in a mismatch between the encoded miR binding site and the corresponding miRNA. In some embodiments, the viral genome comprises at least 2, 3, 4, or 5 copies of the encoded miR182 binding site, e.g., an encoded miR182 binding site series. In some embodiments, the at least 2, 3, 4, or 5 copies (e.g., 2 or 3 copies) of the encoded miR182 binding site are continuous (e.g., not separated by a spacer) or separated by a spacer. In some embodiments, the spacer is about 1 to 6 nucleotides or about 5 to 10 nucleotides, e.g., about 7-8 nucleotides or about 8 nucleotides, in length. In some embodiments, the spacer comprises the nucleotide sequence of GATAGTTA, or a nucleotide sequence having at least one, two, or three modifications, e.g., substitutions (e.g., conservative substitutions), insertions, or deletions, but no more than four modifications, e.g., substitutions (e.g., conservative substitutions), insertions, or deletions, of GATAGTTA. In some embodiments, the spacer sequence comprises one or more of (i) GGAT; (ii) CACGTG; (iii) GCATGC, or a repeat of one or more of (i)-(iii).

In certain embodiments, the encoded miR binding site or the encoded miR binding site series comprises a miR96 binding site sequence. In some embodiments, the encoded miR96 binding site comprises the nucleotide sequence of AGCAAAAATGTGCTAGTGCCAAA (SEQ ID NO: 3677), a sequence having at least 50%, 55%, 60%, 65%, 70%, 75%, 80%, 85%, 90%, at least 95%, at least 99%, or 100% sequence identity, or having at least one, two, three, four, five, six, or seven modifications, e.g., substitutions (e.g., conservative substitutions), insertions, or deletions, but no more than ten modifications, e.g., substitutions (e.g., conservative substitutions), insertions, or deletions, to SEQ ID NO: 3677, e.g., wherein the modification can result in a mismatch between the encoded miR binding site and the corresponding miRNA. In some embodiments, the viral genome comprises at least 2, 3, 4, or 5 copies of the encoded miR96 binding site, e.g., an encoded miR96 binding site series. In some embodiments, the at least 2, 3, 4, or 5 copies (e.g., 2 or 3 copies) of the encoded miR96 binding site are continuous (e.g., not separated by a spacer) or separated by a spacer. In some embodiments, the spacer is about 1 to 6 nucleotides or about 5 to 10 nucleotides, e.g., about 7-8 nucleotides or about 8 nucleotides, in length. In some embodiments, the spacer comprises the nucleotide sequence of GATAGTTA, or a nucleotide sequence having at least one, two, or three modifications, e.g., substitutions (e.g., conservative substitutions), insertions, or deletions, but no more than four modifications, e.g., substitutions (e.g., conservative substitutions), insertions, or deletions, of GATAGTTA. In some embodiments, the spacer sequence comprises one or more of (i) GGAT; (ii) CACGTG; (iii) GCATGC, or a repeat of one or more of (i)-(iii).

In some embodiments, the encoded miR binding site series comprises a miR122 binding site, a miR-1, a miR142 binding site, a miR183 binding site, a miR182 binding site, a miR 96 binding site, or a combination thereof. In some embodiments, the encoded miR binding site series comprises at least 2, 3, 4, or 5 copies of a miR122 binding site, a miR142 binding site, a miR183 binding site, a miR182 binding site, a miR 96 binding site, or a combination thereof. In some embodiments, at least two of the encoded miR binding sites are connected directly, e.g., without a spacer. In other embodiments, at least two of the encoded miR binding sites are separated by a spacer, e.g., 1, 2, 3, 4, 5, 6, 7, 8, 9, or 10 nucleotides in length, which is located between two or more consecutive encoded miR binding site sequences. In embodiments, the spacer is at least about 5 to 10 nucleotides, e.g., about 7-8 nucleotides or about 8 nucleotides, in length. In some embodiments, the spacer coding sequence or reverse complement thereof comprises one or more of (i) GGAT; (ii) CACGTG; (iii) GCATGC, or a repeat of one or more of (i)-(iii). In some embodiments, the spacer comprises the nucleotide sequence of GATAGTTA, or a nucleotide sequence having at least one, two, or three modifications, e.g., substitutions (e.g., conservative substitutions), insertions, or deletions, but no more than four modifications, e.g., substitutions (e.g., conservative substitutions), insertions, or deletions, of GATAGTTA.

In some embodiments, an encoded miR binding site series comprises at least 2-5 copies (e.g., 2 or 3 copies) of a combination of at least two, three, four, five, or all of a miR-1, miR122 binding site, a miR142 binding site, a miR183 binding site, a miR182 binding site, a miR96 binding site, wherein each of the miR binding sites within the series are continuous (e.g., not separated by a spacer) or are separated by a spacer. In some embodiments, the spacer is about 1 to 6 nucleotides or about 5 to 10 nucleotides, e.g., about 7-8 nucleotides or about 8 nucleotides, in length. In some embodiments, the spacer sequence comprises one or more of (i) GGAT; (ii) CACGTG; (iii) GCATGC, or a repeat of one or more of (i)-(iii). In some embodiments, the spacer comprises the nucleotide sequence of GATAGTTA, or a nucleotide sequence having at least one, two, or three modifications, e.g., substitutions (e.g., conservative substitutions), insertions, or deletions, but no more than four modifications, e.g., substitutions (e.g., conservative substitutions), insertions, or deletions, of GATAGTTA.

In some embodiments, an encoded miR binding site series comprises at least 2-5 copies (e.g., 2 or 3 copies) of a combination of a miR-122 binding site and a miR-1 binding site, wherein each of the miR binding sites within the series are continuous (e.g., not separated by a spacer) or are separated by a spacer. In some embodiments, the spacer is about 1 to 6 nucleotides or about 5 to 10 nucleotides, e.g., about 7-8 nucleotides or about 8 nucleotides, in length. In some embodiments, the spacer sequence comprises one or more of (i) GGAT; (ii) CACGTG; (iii) GCATGC, or a repeat of one or more of (i)-(iii). In some embodiments, the spacer comprises the nucleotide sequence of GATAGTTA, or a nucleotide sequence having at least one, two, or three modifications, e.g., substitutions (e.g., conservative substitutions), insertions, or deletions, but no more than four modifications, e.g., substitutions (e.g., conservative substitutions), insertions, or deletions, of GATAGTTA.

### Viral Genome Component: Payloads

In some embodiments, an AAV particle, e.g., an AAV particle for the vectorized delivery of a payload comprises a nucleic acid encoding an anti-tau antibody molecule described herein. In some embodiments, an AAV particle described herein comprises at least two, at least three, or at least 4 payloads. In some embodiments, an AAV particle, e.g., an AAV particle for the vectorized delivery of an antibody molecule described herein (e.g., an anti-tau antibody molecule), comprises a nucleic acid comprising a transgene encoding a payload. In some embodiments, the payload comprises an antibody molecule, e.g., an anti-tau antibody molecule. In some embodiments, the payload comprises a secreted protein, an intracellular protein, an extracellular protein, a membrane protein, a structural protein, a functional protein, or a protein, e.g., a mammalian protein, involved in immune system regulation. In some embodiments, a nucleic acid comprises a transgene encoding an antibody molecule that binds to tau.

In some embodiments, the nucleic acid molecule comprising the transgene encoding a payload further comprises a nucleotide sequence encoding a linker (e.g., a linker connecting a heavy chain variable region (VH) and a light chain variable region (VL) of an antibody molecule) and/or a cleavage site. In some embodiments, the nucleic acid molecule comprising the transgene encoding a payload further comprises a nucleotide sequence encoding a signal sequence.

In some embodiments, the nucleic acid encoding the payload may be constructed, e.g., organized, similar to, e.g., mirroring, the natural organization of an mRNA. In some embodiments, the nucleic acid encoding the payload may comprise coding and/or non-coding nucleotide sequences. In some embodiments, the nucleic acid encoding the payload may encode a coding and/or a non-coding RNA.

In such an embodiment, the nucleic acid comprising a transgene encoding a payload (e.g., an antibody molecule) is replicated and packaged into an AAV particle. In some embodiments, following transduction of a cell with an AAV particle comprising a payload (e.g., an antibody molecule), the cell expresses the payload. In some embodiments, the payload, e.g., antibody molecule, produced by a cell transduced by an AAV particle comprising the payload, is secreted from the cell.

In some embodiments, the payload region may comprise at least one inverted terminal repeat (ITR), a promoter region, an intron region, and a coding region. In some embodiments, the coding region comprises a heavy chain region and/or a light chain region of an antibody molecule, and any two components may be separated by a linker region.

In some embodiments, the coding region may comprise a payload region with a heavy chain and light chain sequence separated by a linker and/or a cleavage site. In some embodiments, the heavy and light chain sequence is separated by an IRES sequence. In some embodiments, the heavy and light chain sequence is separated by a foot and mouth virus sequence. In some embodiments, the heavy and light chain sequence is separated by a foot and mouth virus sequence and a furin cleavage site. In some embodiments, the heavy and light chain sequence is separated by a porcine teschovirus-1 virus sequence. In some embodiments, the heavy and light chain sequence is separated by a porcine teschovirus-1 virus and a furin cleavage site. In some embodiments, the heavy and light chain sequence is separated by a 5xG4S sequence (“5xG4S” disclosed as SEQ ID NO: 5140).

### Antibody Molecules

In some embodiments, the nucleic acid comprises a transgene encoding an antibody molecule. In some embodiments, the encoded antibody molecule binds to tau. For example, the encoded antibody molecule binds to an epitope, e.g. a linear or conformational epitope on tau.

In some embodiments, the encoded antibody molecule comprises at least one immunoglobulin variable domain sequence. An antibody molecule may include, for example, full-length, mature antibodies and antigen-binding fragments of an antibody. For example, an antibody molecule can include a heavy (H) chain variable domain sequence (abbreviated herein as VH), and a light (L) chain variable domain sequence (abbreviated herein as VL). In another example, an antibody molecule includes two heavy (H) chain variable domain sequences and two light (L) chain variable domain sequence, thereby forming two antigen binding sites, such as Fab, Fab′, F(ab′)2, Fc, Fd, Fd′, Fv, single chain antibodies (scFv for example), single variable domain antibodies, diabodies (Dab) (bivalent and bispecific), and chimeric (e.g., humanized) antibodies, which may be produced by the modification of whole antibodies or those synthesized de novo using recombinant DNA technologies. These functional antibody fragments retain the ability to selectively bind with their respective antigen or receptor. Antibodies and antibody fragments can be from any class of antibodies including, but not limited to, IgG, IgA, IgM, IgD, and IgE, and from any subclass (e.g., IgG1, IgG2, IgG3, and IgG4) of antibodies. The encoded antibodies of the present disclosure can be monoclonal or polyclonal. The encoded antibody can also be a human, humanized, CDR-grafted, or in vitro generated antibody. The encoded antibody can have a heavy chain constant region chosen from, e.g., IgG1, IgG2, IgG3, or IgG4. The encoded antibody can also have a light chain chosen from, e.g., kappa or lambda.

Examples of antigen-binding fragments include: (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a diabody (dAb) fragment, which consists of a VH domain; (vi) a camelid or camelized variable domain; (vii) a single chain Fv (scFv), see e.g., Bird et al. (1988) Science 242:423-426; and Huston et al. (1988) Proc. Natl. Acad. Sci. USA 85:5879-5883); and (viii) a single domain antibody. These antibody fragments are obtained using conventional techniques known to those with skill in the art, and the fragments are screened for utility in the same manner as are intact antibodies. An antibody fragment can also be incorporated into single domain antibodies, maxibodies, minibodies, nanobodies, intrabodies, diabodies, triabodies, tetrabodies, v-NAR and bis-scFv (see, for example, Hollinger and Hudson, Nature Biotechnology 23:1126-1136, 2005).

In some embodiments, an encoded antibody molecule of the present disclosure comprises a functional fragment or variant thereof. Constant regions of the antibodies can be altered, e.g., mutated, to modify the properties of the antibody (e.g., to increase or decrease one or more of: Fc receptor binding, antibody glycosylation, the number of cysteine residues, effector cell function, or complement function).

In some embodiments, the encoded antibody molecule can be single domain antibody. Single domain antibodies can include antibodies whose complementary determining regions are part of a single domain polypeptide. Examples include, but are not limited to, heavy chain antibodies, antibodies naturally devoid of light chains, single domain antibodies derived from conventional 4-chain antibodies, engineered antibodies and single domain scaffolds other than those derived from antibodies. Single domain antibodies may be any of the art, or any future single domain antibodies. Single domain antibodies may be derived from any species including, but not limited to mouse, human, camel, llama, fish, shark, goat, rabbit, and bovine. According to another aspect of the invention, a single domain antibody is a naturally occurring single domain antibody known as heavy chain antibody devoid of light chains. Such single domain antibodies are disclosed in WO 9404678, for example. For clarity reasons, this variable domain derived from a heavy chain antibody naturally devoid of light chain is known herein as a VHH or nanobody to distinguish it from the conventional VH of four chain immunoglobulins. Such a VHH molecule can be derived from antibodies raised in Camelidae species, for example in camel, llama, dromedary, alpaca and guanaco. Other species besides Camelidae may produce heavy chain antibodies naturally devoid of light chain; such VHHs are within the scope of the invention.

In some embodiments, the VH and VL regions of the encoded antibody molecule can be subdivided into regions of hypervariability, termed complementarity determining regions (CDR), interspersed with regions that are more conserved, termed framework regions (FR or FW).

The extent of the framework region and CDRs has been precisely defined by a number of methods (see, Kabat, E. A., et al. (1991) Sequences of Proteins of Immunological Interest, Fifth Edition, U.S. Department of Health and Human Services, NIH Publication No. 91-3242; Chothia, C. et al. (1987) J. Mol. Biol. 196:901-917; and the AbM definition used by Oxford Molecular's AbM antibody modeling software. See, generally, e.g., Protein Sequence and Structure Analysis of Antibody Variable Domains. In: Antibody Engineering Lab Manual (Ed.: Duebel, S. and Kontermann, R., Springer-Verlag, Heidelberg).

Complementarity determining region, and CDR, as used herein refer to the sequences of amino acids within antibody variable regions which confer antigen specificity and binding affinity. In general, there are three CDRs in each heavy chain variable region (HCDR1, HCDR2, HCDR3) and three CDRs in each light chain variable region (LCDR1, LCDR2, LCDR3).

The precise amino acid sequence boundaries of a given CDR can be determined using any of a number of well-known schemes, including those described by Kabat et al. (1991), “Sequences of Proteins of Immunological Interest,” 5th Ed. Public Health Service, National Institutes of Health, Bethesda, MD (Kabat numbering scheme), Al-Lazikani et al., (1997) JMB 273,927-948 (Chothia numbering scheme). In some embodiments, the CDRs defined according the Chothia number scheme are also sometimes referred to as hypervariable loops.

For example, under Kabat, the CDR amino acid residues in the heavy chain variable domain (VH) are numbered 31-35 (HCDR1), 50-65 (HCDR2), and 95-102 (HCDR3); and the CDR amino acid residues in the light chain variable domain (VL) are numbered 24-34 (LCDR1), 50-56 (LCDR2), and 89-97 (LCDR3). Under Chothia the CDR amino acids in the VH are numbered 26-32 (HCDR1), 52-56 (HCDR2), and 95-102 (HCDR3); and the amino acid residues in VL are numbered 26-32 (LCDR1), 50-52 (LCDR2), and 91-96 (LCDR3). By combining the CDR definitions of both Kabat and Chothia, the CDRs consist of amino acid residues 26-35 (HCDR1), 50-65 (HCDR2), and 95-102 (HCDR3) in human VH and amino acid residues 24-34 (LCDR1), 50-56 (LCDR2), and 89-97 (LCDR3) in human VL.

In some embodiments, the antigen binding domain of the encoded antibody molecules of the present disclosure is the part of the encoded antibody molecule that comprises determinants that form an interface that binds to the tau protein or an epitope thereof. With respect to proteins (or protein mimetics), the antigen-binding site typically includes one or more loops (of at least four amino acids or amino acid mimics) that form an interface that binds to the tau protein. Typically, the antigen-binding site of an antibody molecule includes at least one or two CDRs and/or hypervariable loops, or more typically at least three, four, five or six CDRs and/or hypervariable loops.

The encoded antibody molecule can be a monoclonal antibody molecule or a polyclonal antibody molecule. In some embodiments, a monoclonal antibody or a monoclonal antibody composition refer to a preparation of antibody molecules of single molecular composition. A monoclonal antibody composition displays a single binding specificity and affinity for a particular epitope. A monoclonal antibody can be made by hybridoma technology or by methods that do not use hybridoma technology (e.g., recombinant methods).

In some embodiments, the sequences of an antibody molecule to be included in an encoded payload described herein can be generated by recombinant libraries, e.g., generated by phage display or by combinatorial methods.

Phage display and combinatorial methods for generating antibodies are known in the art (as described in, e.g., Ladner et al. U.S. Pat. No. 5,223,409; Kang et al. International Publication No. WO 92/18619; Dower et al. International Publication No. WO 91/17271; Winter et al. International Publication WO 92/20791; Markland et al. International Publication No. WO 92/15679; Breitling et al. International Publication WO 93/01288; McCafferty et al. International Publication No. WO 92/01047; Garrard et al. International Publication No. WO 92/09690; Ladner et al. International Publication No. WO 90/02809; Fuchs et al. (1991) Bio/Technology 9:1370-1372; Hay et al. (1992) Hum Antibod Hybridomas 3:81-85; Huse et al. (1989) Science 246:1275-1281; Griffths et al. (1993) EMBO J 12:725-734; Hawkins et al. (1992) J Mol Biol 226:889-896; Clackson et al. (1991) Nature 352:624-628; Gram et al. (1992) PNAS 89:3576-3580; Garrad et al. (1991) Bio/Technology 9:1373-1377; Hoogenboom et al. (1991) Nuc Acid Res 19:4133-4137; and Barbas et al. (1991) PNAS 88:7978-7982, the contents of all of which are incorporated by reference herein).

In some embodiments, the sequences of an antibody molecule to be included in an encoded payload described herein can be generated from an antibody molecule that is designed using the VERSITOPE™ Antibody Generation or BIOATLA®, e.g., in US20130303399, US20130281303, WO2012009026, WO2016033331, WO2016036916, and U.S. Pat. No. 8,859,467, the contents of which are herein incorporated by reference in their entirety. In some embodiments, the sequences of an antibody molecule to be included in an encoded payload described herein can be derived from an antibody molecule that is designed and/or produced using the methods described, e.g., in WO2017189959 and WO2020223276, the contents of which are herein incorporated by reference in their entirety.

In some embodiments, the encoded antibody comprises an amino acid sequence of a fully human antibody (e.g., an antibody made in a mouse which has been genetically engineered to produce an antibody from a human immunoglobulin sequence), or a non-human antibody, e.g., a rodent (mouse or rat), goat, primate (e.g., monkey), camel antibody. Preferably, the non-human antibody is a rodent (mouse or rat antibody). Methods of producing rodent antibodies are known in the art.

Human monoclonal antibodies can be generated using transgenic mice carrying the human immunoglobulin genes rather than the mouse system. Splenocytes from these transgenic mice immunized with the antigen of interest are used to produce hybridomas that secrete human mAbs with specific affinities for epitopes from a human protein (see, e.g., Wood et al. International Application WO 91/00906, Kucherlapati et al. PCT publication WO 91/10741; Lonberg et al. International Application WO 92/03918; Kay et al. International Application 92/03917; Lonberg, N. et al. 1994 Nature 368:856-859; Green, L. L. et al. 1994 Nature Genet. 7:13-21; Morrison, S. L. et al. 1994 Proc. Natl. Acad. Sci. USA 81:6851-6855; Bruggeman et al. 1993 Year Immunol 7:33-40; Tuaillon et al. 1993 PNAS 90:3720-3724; Bruggeman et al. 1991 Eur J Immunol 21:1323-1326).

In some embodiments, the encoded antibody comprises an amino acid sequence of an antibody in which the variable region, or a portion thereof, e.g., the CDRs, are generated in a non-human organism, e.g., a rat or mouse. Encoded antibody molecules comprising chimeric, CDR-grafted, and humanized antibodies are within the invention. Encoded antibody molecules comprising the sequences of antibodies generated in a non-human organism, e.g., a rat or mouse, and then modified, e.g., in the variable framework or constant region, to decrease antigenicity in a human are within the invention.

An effectively human protein is a protein that does substantially not evoke a neutralizing antibody response, e.g., the human anti-murine antibody (HAMA) response. HAMA can be problematic in a number of circumstances, e.g., if the antibody molecule is administered repeatedly, e.g., in treatment of a chronic or recurrent disease condition. A HAMA response can make repeated antibody administration potentially ineffective because of an increased antibody clearance from the serum (see, e.g., Saleh et al., Cancer Immunol. Immunother., 32:180-190 (1990)) and also because of potential allergic reactions (see, e.g., LoBuglio et al., Hybridoma, 5:5117-5123 (1986)).

Chimeric antibodies can be produced by recombinant DNA techniques known in the art (see Robinson et al., International Patent Publication PCT/US86/02269; Akira, et al., European Patent Application 184,187; Taniguchi, M., European Patent Application 171,496; Morrison et al., European Patent Application 173,494; Neuberger et al., International Application WO 86/01533; Cabilly et al. U.S. Pat. No. 4,816,567; Cabilly et al., European Patent Application 125,023; Better et al. (1988 Science 240:1041-1043); Liu et al. (1987) PNAS 84:3439-3443; Liu et al., 1987, J. Immunol. 139:3521-3526; Sun et al. (1987) PNAS 84:214-218; Nishimura et al., 1987, Canc. Res. 47:999-1005; Wood et al. (1985) Nature 314:446-449; and Shaw et al., 1988, J. Natl Cancer Inst. 80:1553-1559).

A humanized or CDR-grafted antibody will have at least one or two but generally all three recipient CDRs (of heavy and or light immuoglobulin chains) replaced with a donor CDR. The antibody may be replaced with at least a portion of a non-human CDR or only some of the CDRs may be replaced with non-human CDRs. It is only necessary to replace the number of CDRs required for binding of the humanized antibody to the target, e.g., tau. Preferably, the donor will be a rodent antibody, e.g., a rat or mouse antibody, and the recipient will be a human framework or a human consensus framework. Typically, the immunoglobulin providing the CDRs is called the donor and the immunoglobulin providing the framework is called the acceptor. In some embodiments, the donor immunoglobulin is a non-human (e.g., rodent). The acceptor framework is a naturally-occurring (e.g., a human) framework or a consensus framework, or a sequence about 85% or higher, preferably 90%, 95%, 99% or higher identical thereto.

In some embodiments, the consensus sequence refers to the sequence formed from the most frequently occurring amino acids (or nucleotides) in a family of related sequences (See e.g., Winnaker, From Genes to Clones (Verlagsgesellschaft, Weinheim, Germany 1987). In a family of proteins, each position in the consensus sequence is occupied by the amino acid occurring most frequently at that position in the family. If two amino acids occur equally frequently, either can be included in the consensus sequence. In some embodiments, the consensus framework refers to the framework region in the consensus immunoglobulin sequence.

An antibody can be humanized by methods known in the art (see e.g., Morrison, S. L., 1985, Science 229:1202-1207, by Oi et al., 1986, BioTechniques 4:214, and by Queen et al. U.S. Pat. Nos. 5,585,089, 5,693,761 and 5,693,762, the contents of all of which are hereby incorporated by reference).

Humanized or CDR-grafted antibodies can be produced by CDR-grafting or CDR substitution, wherein one, two, or all CDRs of an immunoglobulin chain can be replaced. See e.g., U.S. Pat. No. 5,225,539; Jones et al. 1986 Nature 321:552-525; Verhoeyan et al. 1988 Science 239:1534; Beidler et al. 1988 J. Immunol. 141:4053-4060; Winter U.S. Pat. No. 5,225,539, the contents of all of which are hereby expressly incorporated by reference. Winter describes a CDR-grafting method which may be used to prepare the humanized antibodies of the present invention (UK Patent Application GB 2188638A, filed on Mar. 26, 1987; Winter U.S. Pat. No. 5,225,539), the contents of which is expressly incorporated by reference.

In some embodiments, the encoded antibodies comprise the sequences of humanized antibodies in which specific amino acids have been substituted, deleted or added. Criteria for selecting amino acids from the donor are described in U.S. Pat. No. 5,585,089, e.g., columns 12-16 of U.S. Pat. No. 5,585,089, e.g., columns 12-16 of U.S. Pat. No. 5,585,089, the contents of which are hereby incorporated by reference. Other techniques for humanizing antibodies are described in Padlan et al. EP 519596 A1, published on Dec. 23, 1992.

In some embodiments, the encoded antibody molecule can be a single chain antibody. A single-chain antibody (scFV) may be engineered (see, for example, Colcher, D. et al. (1999) Ann N Y Acad Sci 880:263-80; and Reiter, Y. (1996) Clin Cancer Res 2:245-52). The single chain antibody can be dimerized or multimerized to generate multivalent antibodies having specificities for different epitopes of the same target protein.

In yet other embodiments, the antibody molecule has a heavy chain constant region chosen from, e.g., the heavy chain constant regions of IgG1, IgG2, IgG3, IgG4, IgM, IgA1, IgA2, IgD, and IgE; particularly, chosen from, e.g., the (e.g., human) heavy chain constant regions of IgG1, IgG2, IgG3, and IgG4. In another embodiment, the antibody molecule has a light chain constant region chosen from, e.g., the (e.g., human) light chain constant regions of kappa or lambda. The constant region can be altered, e.g., mutated, to modify the properties of the antibody (e.g., to increase or decrease one or more of: Fc receptor binding, antibody glycosylation, the number of cysteine residues, effector cell function, and/or complement function). In some embodiments the antibody has: effector function; and can fix complement. In other embodiments the antibody does not recruit effector cells; or fix complement. In other embodiments, the antibody has reduced or no ability to bind an Fc receptor. For example, it is an isotype or subtype, fragment or other mutant, which does not support binding to an Fc receptor, e.g., it has a mutagenized or deleted Fc receptor binding region.

Methods for altering an antibody constant region are known in the art. Antibodies with altered function, e.g. altered affinity for an effector ligand, such as FcR on a cell, or the C1 component of complement can be produced by replacing at least one amino acid residue in the constant portion of the antibody with a different residue (see e.g., EP 388,151 A1, U.S. Pat. Nos. 5,624,821 and 5,648,260, the contents of all of which are hereby incorporated by reference). Similar type of alterations could be described which if applied to the murine, or other species immunoglobulin would reduce or eliminate these functions.

An antibody molecule can be derivatized or linked to another functional molecule (e.g., another peptide or protein). In some embodiments, a derivatized antibody molecule is one that has been modified. Methods of derivatization include but are not limited to the addition of a fluorescent moiety, a radionucleotide, a toxin, an enzyme or an affinity ligand such as biotin. In some embodiments, the nucleic acid sequence comprising the transgene encoding the antibody molecule further encodes a fluorescent moiety, a radionucleotide, a toxin, an enzyme or an affinity ligand such as biotin. Accordingly, the encoded antibody molecules of the invention are intended to include derivatized and otherwise modified forms of the antibodies described herein, including immunoadhesion molecules. For example, an encoded antibody molecule can be functionally linked (by genetic fusion or otherwise) to one or more other molecular entities, such as another antibody (e.g., a bispecific antibody or a diabody), a detectable agent, a cytotoxic agent, a pharmaceutical agent, and/or a protein or peptide that can mediate association of the antibody or antibody portion with another molecule (such as a streptavidin core region or a polyhistidine tag). In some embodiments, the nucleic acid sequence comprising the transgene encoding the antibody molecule further encodes a another antibody (e.g., a bispecific antibody or a diabody), a detectable agent, a cytotoxic agent, a pharmaceutical agent, and/or a protein or peptide that can mediate association of the antibody or antibody portion with another molecule (e.g., a tag, e.g., a tag described herein).

Payload regions may encode polypeptides that form or function as any antibody, including antibodies that are known in the art and/or antibodies that are commercially available. The encoded antibodies may be therapeutic, diagnostic, or for research purposes. Further, polypeptides may include fragments of such antibodies or antibodies that have been developed to comprise one or more of such fragments (e.g., variable domains or complementarity determining regions (CDRs))

### Multispecific Antibody Molecules

In some embodiments, the encoded antibody molecule is a multispecific antibody molecule, e.g., it comprises a plurality of immunoglobulin variable domains sequences, wherein a first immunoglobulin variable domain sequence of the plurality has binding specificity for a first epitope and a second immunoglobulin variable domain sequence of the plurality has binding specificity for a second epitope. In some embodiments, the first and second epitopes are on the same antigen, e.g., the same protein (or subunit of a multimeric protein). In some embodiments, the first and second epitopes overlap. In some embodiments, the first and second epitopes do not overlap. In some embodiments, the first and second epitopes are on different antigens, e.g., the different proteins (or different subunits of a multimeric protein). In some embodiments, a multispecific antibody molecule comprises a third, fourth or fifth immunoglobulin variable domain. In some embodiments, a multispecific antibody molecule is a bispecific antibody molecule, a trispecific antibody molecule, or tetraspecific antibody molecule. In some embodiments, the encoded anti-tau molecule is a multispecific antibody molecule.

In some embodiments, an encoded multispecific antibody molecule is an encoded bispecific antibody molecule. A bispecific antibody has specificity for no more than two antigens. A bispecific antibody molecule is characterized by a first immunoglobulin variable domain sequence which has binding specificity for a first epitope and a second immunoglobulin variable domain sequence that has binding specificity for a second epitope. In some embodiments, the first and second epitopes are on the same antigen, e.g., the same protein (or subunit of a multimeric protein). In some embodiments, the first and second epitopes overlap. In some embodiments, the first and second epitopes do not overlap. In some embodiments, the first and second epitopes are on different antigens, e.g., the different proteins (or different subunits of a multimeric protein). In some embodiments, a bispecific antibody molecule comprises a heavy chain variable domain sequence and a light chain variable domain sequence which have binding specificity for a first epitope and a heavy chain variable domain sequence and a light chain variable domain sequence which have binding specificity for a second epitope. In some embodiments, a bispecific antibody molecule comprises a half antibody having binding specificity for a first epitope and a half antibody having binding specificity for a second epitope. In some embodiments, a bispecific antibody molecule comprises a half antibody, or fragment thereof, having binding specificity for a first epitope and a half antibody, or fragment thereof, having binding specificity for a second epitope. In some embodiments, a bispecific antibody molecule comprises a scFv, or fragment thereof, have binding specificity for a first epitope and a scFv, or fragment thereof, have binding specificity for a second epitope. In some embodiment, the encoded anti-tau antibody is a bispecific antibody molecule.

In another aspect, the invention relates to a multispecific, e.g., bispecific (e.g., biparatopic), antibody molecule comprising one or more tau antigen binding domains, such as but not limited to an anti-tau antibody molecule or fragment comprising one or more CDRs, VH, heavy chain, VL, and/or light chain thereof. Non-limiting anti-tau antibody molecule sequences are provided in Tables 7-16.

In some embodiments, the antibody molecule encoded by a transgene described herein is an anti-tau bispecific antibody molecule. In some embodiments, the anti-tau bispecific antibody molecule is monovalent or bivalent. In some embodiments, the anti-tau bispecific antibody molecule is a biparatopic antibody molecule. In some embodiments, the anti-tau bispecific antibody molecule comprises an Fc region or a variant thereof, e.g., as provided in Table 17. In some embodiments, the Fc region has reduced, e.g., ablated, affinity for an Fc receptor, e.g., an Fc receptor described herein. In some embodiments, the reduced affinity is compared to an otherwise similar antibody with a wild type Fc region. In some embodiments, the Fc receptor comprises a mutation at one or more of (e.g., all of) positions I253 (e.g., 1235A), H310 (e.g., H310A or H310Q), and/or H435 (e.g., H435A or H435Q), numbered according to the EU index as in Kabat. In some embodiments, a bispecific antibody molecule described herein comprises a variant Fc region with reduced effector function (e.g., reduced ADCC). In some embodiments, the Fc region comprises a mutation at one or more of (e.g., all of) positions L235 (e.g., L235V), F243 (e.g., F243L), R292 (e.g., R292P), Y300 (e.g., Y300L), and P396 (e.g., P396L), numbered according to the EU index as in Kabat.

In some embodiments, the first and/or second antigen binding domain comprise an IgG antibody, single-chain Fv (scFv), a scFv fragment, a Fab, a single-chain Fab (scFabs), a single-chain antibody, a diabody, an antibody variable domain, a VHH, a single domain antibody, and/or a nanobody.

In some embodiments, the first antigen binding domain (e.g., an scFv) is located at the N-terminus of the VL of the second binding domain (e.g., a full antibody, e.g., an IgG antibody), e.g., linked via a peptide linker. In some embodiments, a polypeptide of the bispecific antibody molecule comprises the following sequences, from N-terminal to C-terminal: VH of the first binding domain, first peptide linker (e.g., a (G4S)3 linker), VL of first binding domain, second peptide linker (e.g., a (G4S) linker), VL of the second binding domain and CL. In some embodiments, a polypeptide of the bispecific antibody molecule comprises the following sequences: from N-terminal to C-terminal: VH of the second binding domain, CH1, CH2, and CH3. In some embodiments, the bispecific antibody molecule comprises an Fc region that is mutated to have reduced binding to Fc receptor or reduced ADCC, e.g., an Fc region having the mutations L235V, F243L, R292P, Y300L, and P396L, numbered according to the EU index as in Kabat.

In some embodiments, the first antigen binding domain (e.g., an antibody mimetic, e.g., a designed ankyrin repeat protein (DARPIN)) is located at the N-terminus of the VL of the second binding domain (e.g., a full antibody, e.g., an IgG antibody), e.g., linked via a peptide linker. In some embodiments, a polypeptide of the bispecific antibody molecule comprises the following sequences, from N-terminal to C-terminal: an antibody mimetic, e.g., a designed ankyrin repeat protein (DARPIN), a peptide linker (e.g., a (G4S)3 linker), VL of the second binding domain and CL. In some embodiments, a polypeptide of the bispecific antibody molecule comprises the following sequences: from N-terminal to C-terminal: VH of the second binding domain, CH1, CH2, and CH3. In some embodiments, the bispecific antibody molecule comprises an Fc region that is mutated to have reduced binding to Fc receptor or reduced ADCC, e.g., an Fc region having the mutations L235V, F243L, R292P, Y300L, and P396L, numbered according to the EU index as in Kabat.

In some embodiments, the first antigen binding domain (e.g., a Fyn SH3-derived binding polypeptide (e.g., a fynomer)) is located at the N-terminus of the VL of the second binding domain (e.g., a full antibody, e.g., an IgG antibody), e.g., linked via a peptide linker. In some embodiments, a polypeptide of the bispecific antibody molecule comprises the following sequences, from N-terminal to C-terminal: a Fyn SH3-derived binding polypeptide, a peptide linker (e.g., a (G4S)3 linker), VL of the second binding domain and CL. In some embodiments, a polypeptide of the bispecific antibody molecule comprises the following sequences: from N-terminal to C-terminal: VH of the second binding domain, CH1, CH2, and CH3. In some embodiments, the bispecific antibody molecule comprises an Fc region that is mutated to have reduced binding to Fc receptor or reduced ADCC, e.g., an Fc region having the mutations L235V, F243L, R292P, Y300L, and P396L, numbered according to the EU index as in Kabat.

In a further aspect, the invention relates to a bispecific molecule comprising a first antigen binding site from a tau antibody of the invention as described herein above and a second antigen binding site with a different binding specificity, such as a binding specificity for a human effector cell, a human Fc receptor, a T cell receptor, a B cell receptor or a binding specificity for a non-overlapping epitope of tau, i.e. a bispecific antibody wherein the first and second antigen binding sites do not cross-block each other for binding to tau.

Exemplary bispecific antibody molecules of the invention comprise (i) two antibodies, one with a specificity to tau and another to a second target that are conjugated together, (ii) a single antibody that has one chain or arm specific to tau and a second chain or arm specific to a second molecule, (iii) two antibodies, one with a specificity for a first epitope of tau and another to a second epitope of tau that are conjugated together, wherein the first and second epitopes are distinct epitopes, (iv) a single antibody that has one chain or arm specific to a first epitope of tau and a second chain or arm specific to a second epitope of tau, wherein the first and second epitopes are distinct epitopes, (v) a single chain antibody that has specificity to tau and a second molecule (or another epitope of tau), e.g., via two scFvs linked in tandem by an extra peptide linker; (vi) a dual-variable-domain antibody (DVD-Ig), where each light chain and heavy chain contains two variable domains in tandem through a short peptide linkage (Wu et al., Generation and Characterization of a Dual Variable Domain Immunoglobulin (DVD-Ig™) Molecule, In: Antibody Engineering, Springer Berlin Heidelberg (2010)); (vii) a chemically-linked bispecific (Fab′)2 fragment; (viii) a Tandab, which is a fusion of two single chain diabodies resulting in a tetravalent bispecific antibody that has two binding sites for each of the target antigens; (ix) a flexibody, which is a combination of scFvs with a diabody resulting in a multivalent molecule; (x) a so called “dock and lock” molecule, based on the “dimerization and docking domain” in Protein Kinase A, which, when applied to Fabs, can yield a trivalent bispecific binding protein consisting of two identical Fab fragments linked to a different Fab fragment; (xi) a so-called Scorpion molecule, comprising, e.g., two scFvs fused to both termini of a human Fc-region; and (xii) a diabody. In one embodiment, the bispecific antibody of the present invention is a diabody.

In some embodiments, the sequences of the encoded antibody molecules of the present disclosure can be generated from bispecific or heterodimeric antibody molecules produced using protocols known in the art; including but not limited to, for example, the “knob in a hole” approach described in, e.g., U.S. Pat. No. 5,731,168; the electrostatic steering Fc pairing as described in, e.g., WO 09/089004, WO 06/106905 and WO 2010/129304; Strand Exchange Engineered Domains (SEED) heterodimer formation as described in, e.g., WO 07/110205; Fab arm exchange as described in, e.g., WO 08/119353, WO 2011/131746, and WO 2013/060867; double antibody conjugate, e.g., by antibody cross-linking to generate a bi-specific structure using a heterobifunctional reagent having an amine-reactive group and a sulfhydryl reactive group as described in, e.g., U.S. Pat. No. 4,433,059; bispecific antibody determinants generated by recombining half antibodies (heavy-light chain pairs or Fabs) from different antibodies through cycle of reduction and oxidation of disulfide bonds between the two heavy chains, as described in, e.g., U.S. Pat. No. 4,444,878; trifunctional antibodies, e.g., three Fab′ fragments cross-linked through sulfhdryl reactive groups, as described in, e.g., U.S. Pat. No. 5,273,743; biosynthetic binding proteins, e.g., pair of scFvs cross-linked through C-terminal tails preferably through disulfide or amine-reactive chemical cross-linking, as described in, e.g., U.S. Pat. No. 5,534,254; bifunctional antibodies, e.g., Fab fragments with different binding specificities dimerized through leucine zippers (e.g., c-fos and c-jun) that have replaced the constant domain, as described in, e.g., U.S. Pat. No. 5,582,996; bispecific and oligospecific mono- and oligovalent receptors, e.g., VH-CH1 regions of two antibodies (two Fab fragments) linked through a polypeptide spacer between the CH1 region of one antibody and the VH region of the other antibody typically with associated light chains, as described in, e.g., U.S. Pat. No. 5,591,828; bispecific DNA-antibody conjugates, e.g., crosslinking of antibodies or Fab fragments through a double stranded piece of DNA, as described in, e.g., U.S. Pat. No. 5,635,602; bispecific fusion proteins, e.g., an expression construct containing two scFvs with a hydrophilic helical peptide linker between them and a full constant region, as described in, e.g., U.S. Pat. No. 5,637,481; multivalent and multispecific binding proteins, e.g., dimer of polypeptides having first domain with binding region of Ig heavy chain variable region, and second domain with binding region of Ig light chain variable region, generally termed diabodies (higher order structures are also disclosed creating bispecific, trispecific, or tetraspecific molecules, as described in, e.g., U.S. Pat. No. 5,837,242; minibody constructs with linked VL and VH chains further connected with peptide spacers to an antibody hinge region and CH3 region, which can be dimerized to form bispecific/multivalent molecules, as described in, e.g., U.S. Pat. No. 5,837,821; VH and VL domains linked with a short peptide linker (e.g., 5 or 10 amino acids) or no linker at all in either orientation, which can form dimers to form bispecific diabodies; trimers and tetramers, as described in, e.g., U.S. Pat. No. 5,844,094; String of VH domains (or VL domains in family members) connected by peptide linkages with crosslinkable groups at the C-terminus further associated with VL domains to form a series of FVs (or scFvs), as described in, e.g., U.S. Pat. No. 5,864,019; and single chain binding polypeptides with both a VH and a VL domain linked through a peptide linker are combined into multivalent structures through non-covalent or chemical crosslinking to form, e.g., homobivalent, heterobivalent, trivalent, and tetravalent structures using both scFV or diabody type format, as described in, e.g., U.S. Pat. No. 5,869,620. Additional exemplary multispecific and bispecific molecules and methods of making the same are found, for example, in U.S. Pat. Nos. 5,910,573, 5,932,448, 5,959,083, 5,989,830, 6,005,079, 6,239,259, 6,294,353, 6,333,396, 6,476,198, 6,511,663, 6,670,453, 6,743,896, 6,809,185, 6,833,441, 7,129,330, 7,183,076, 7,521,056, 7,527,787, 7,534,866, 7,612,181, US2002/004587A1, US2002/076406A1, US2002/103345A1, US2003/207346A1, US2003/211078A1, US2004/219643A1, US2004/220388A1, US2004/242847A1, US2005/003403A1, US2005/004352A1, US2005/069552A1, US2005/079170A1, US2005/100543A1, US2005/136049A1, US2005/136051A1, US2005/163782A1, US2005/266425A1, US2006/083747A1, US2006/120960A1, US2006/204493A1, US2006/263367A1, US2007/004909A1, US2007/087381A1, US2007/128150A1, US2007/141049A1, US2007/154901A1, US2007/274985A1, US2008/050370A1, US2008/069820A1, US2008/152645A1, US2008/171855A1, US2008/241884A1, US2008/254512A1, US2008/260738A1, US2009/130106A1, US2009/148905A1, US2009/155275A1, US2009/162359A1, US2009/162360A1, US2009/175851A1, US2009/175867A1, US2009/232811A1, US2009/234105A1, US2009/263392A1, US2009/274649A1, EP346087A2, WO00/06605A2, WO02/072635A2, WO04/081051A1, WO06/020258A2, WO2007/044887A2, WO2007/095338A2, WO2007/137760A2, WO2008/119353A1, WO2009/021754A2, WO2009/068630A1, WO91/03493A1, WO93/23537A1, WO94/09131A1, WO94/12625A2, WO95/09917A1, WO96/37621A2, WO99/64460A1. The contents of the above-referenced applications are incorporated herein by reference in their entireties.

In some embodiments, the encoded antibody molecule is a bispecific Fynomer-antibody fusion protein, e.g., it comprises a plurality of immunoglobulin variable domains sequences with one of more Fynomers fused to any of the light- or heavy-chain termini, wherein a first immunoglobulin variable domain sequence of the plurality has binding specificity for a first epitope and the Fynomer has binding specificity for a second epitope. In some embodiments, the first and second epitopes are on the same antigen, e.g., the same protein (or subunit of a multimeric protein). In some embodiments, the first and second epitopes overlap. In some embodiments, the first and second epitopes do not overlap. In some embodiments, the first and second epitopes are on different antigens, e.g., the different proteins (or different subunits of a multimeric protein). In some embodiments, the encoded anti-tau molecule is a multispecific Fynomer-antibody fusion molecule. Fynomers that bind a target of interest can routinely be selected using recombinant technology as described for example, in Grbulovski et al., J. Biol. Chem. 282(5) 3196-3204 (2007) and WO 2008/022759. Methods for forming Fynomer-antibody fusion proteins are known in the art including, for example, Brack et al. (2014), Mol Cancer Ther 13(8):2030-2039, US2017/0281768, US2015/0105285A1, WO2014/170063A1, WO2015/141862A1, WO2013/135588A1, U.S. Ser. No. 10/996,226B2, U.S. Pat. No. 9,989,536B2, U.S. Pat. No. 9,689,879B2, U.S. Pat. No. 9,513,296B2, U.S. Ser. No. 10/323,095B2, and U.S. Pat. No. 9,593,314B2.

This present disclosure provides in some embodiments, a nucleic acid (e.g., an isolated nucleic acid) comprising a transgene encoding a payload comprising any of the above antibody molecules and genetic elements, AAV vectors, and AAV particles comprising the same.

### Chimeric Antigen Receptors

In some embodiments, the nucleic acid comprises a transgene encoding a payload (e.g., an antibody molecule that can be used to generate a chimeric antigen receptor (CAR) or a cell expressing a CAR (e.g., a CAR-expressing cell). The CAR may comprise i) an extracellular antigen binding domain, ii) a transmembrane domain, and iii) an intracellular signaling domain (which may comprise one or both of a primary signaling domain and a costimulatory domain). The CAR may further comprise a leader sequence and/or a hinge sequence. In specific embodiments, the CAR construct comprises a scFv domain, wherein the scFv may be preceded by an optional leader sequence, and followed by an optional hinge sequence, a transmembrane region, and an intracellular signaling domain, e.g., wherein the domains are contiguous with and in the same reading frame to form a single fusion protein.

The antigen binding domain of the CAR can be any domain that binds to the antigen including but not limited to a monoclonal antibody, a polyclonal antibody, a recombinant antibody, a human antibody, a humanized antibody, and a functional fragment thereof, including but not limited to a single-domain antibody such as a heavy chain variable domain (VH), a light chain variable domain (VL) and a variable domain (VHH) of camelid derived nanobody, and to an alternative scaffold known in the art to function as antigen binding domain, such as a recombinant fibronectin domain, a T cell receptor (TCR), or a fragment there of, e.g., single chain TCR, and the like. In some instances, it is beneficial for the antigen binding domain to be derived from the same species in which the CAR will ultimately be used in. For example, for use in humans, it may be beneficial for the antigen binding domain of the CAR to comprise human or humanized residues for the antigen binding domain of an antibody or antibody fragment. In some embodiments, the antigen binding domain of the CAR is an scFv antibody fragment that is humanized compared to the murine sequence of the scFv from which it is derived.

In some embodiments, a CAR can be used in the treatment of disease categories which include for example, cancer, autoimmune disorders, B-cell mediated diseases, inflammatory diseases, neuronal disorders, cardiovascular disease and circulatory disorders, or infectious diseases.

### Miniaturized Antibody

In some embodiments, the antibody encoded by the payloads may be a “miniaturized” antibody. Among the best examples of mAb miniaturization are the small modular immunopharmaceuticals (SMIPs) from Trubion Pharmaceuticals. These molecules, which can be monovalent or bivalent, are recombinant single-chain molecules containing one VL, one VH antigen-binding domain, and one or two constant “effector” domains, all connected by linker domains. Presumably, such a molecule might offer the advantages of increased tissue or tumor penetration claimed by fragments while retaining the immune effector functions conferred by constant domains. At least three “miniaturized” SMIPs have entered clinical development. TRU-015, an anti-CD20 SMIP developed in collaboration with Wyeth, is the most advanced project, having progressed to Phase 2 for rheumatoid arthritis (RA). Earlier attempts in systemic lupus erythrematosus (SLE) and B cell lymphomas were ultimately discontinued. Trubion and Facet Biotechnology are collaborating in the development of TRU-016, an anti-CD37 SMIP, for the treatment of CLL and other lymphoid neoplasias, a project that has reached Phase 2. Wyeth has licensed the anti-CD20 SMIP SBI-087 for the treatment of autoimmune diseases, including RA, SLE, and possibly multiple sclerosis, although these projects remain in the earliest stages of clinical testing. (Nelson, A. L., MAbs. 2010. Jan-Feb; 2(1):77-83).

### Diabody

In some embodiments, payloads may encode diabodies. Diabodies are functional bispecific single-chain antibodies (bscAb). These bivalent antigen-binding molecules are composed of non-covalent dimers of scFvs, and can be produced in mammalian cells using recombinant methods. (See, e.g., Mack et al, Proc. Natl. Acad. Sci., 92: 7021-7025, 1995). Few diabodies have entered clinical development. An iodine-123-labeled diabody version of the anti-CEA chimeric antibody cT84.66 has been evaluated for pre-surgical immunoscintigraphic detection of colorectal cancer in a study sponsored by the Beckman Research Institute of the City of Hope (Clinicaltrials.gov NCT00647153) (Nelson, A. L., MAbs., 2010. Jan-Feb; 2(1):77-83).

### Unibody

In some embodiments, payloads may encode a “unibody,” in which the hinge region has been removed from IgG4 molecules. While IgG4 molecules are unstable and can exchange light-heavy chain heterodimers with one another, deletion of the hinge region prevents heavy chain-heavy chain pairing entirely, leaving highly specific monovalent light/heavy heterodimers, while retaining the Fc region to ensure stability and half-life in vivo. This configuration may minimize the risk of immune activation or oncogenic growth, as IgG4 interacts poorly with FcRs and monovalent unibodies fail to promote intracellular signaling complex formation. These contentions are, however, largely supported by laboratory, rather than clinical, evidence. Other antibodies may be “miniaturized” antibodies, which are compacted 100 kDa antibodies (see, e.g., Nelson, A. L., MAbs., 2010. Jan-Feb; 2(1):77-83).

### Intrabody

In some embodiments, payloads may encode intrabodies. Intrabodies are a form of antibody that is not secreted from a cell in which it is produced, but instead targets one or more intracellular proteins. Intrabodies are expressed and function intracellularly, and may be used to affect a multitude of cellular processes including, but not limited to intracellular trafficking, transcription, translation, metabolic processes, proliferative signaling and cell division. In some embodiments, methods described herein include intrabody-based therapies. In some such embodiments, variable domain sequences and/or CDR sequences disclosed herein are incorporated into one or more constructs for intrabody-based therapy. For example, intrabodies may target one or more glycated intracellular proteins or may modulate the interaction between one or more glycated intracellular proteins and an alternative protein.

More than two decades ago, intracellular antibodies against intracellular targets were first described (Biocca, Neuberger and Cattaneo EMBO J. 9: 101-108, 1990). The intracellular expression of intrabodies in different compartments of mammalian cells allows blocking or modulation of the function of endogenous molecules (Biocca, et al., EMBO J. 9: 101-108, 1990; Colby et al., Proc. Natl. Acad. Sci. U.S.A. 101: 17616-21, 2004). Intrabodies can alter protein folding, protein-protein, protein-DNA, protein-RNA interactions and protein modification. They can induce a phenotypic knockout and work as neutralizing agents by direct binding to the target antigen, by diverting its intracellular trafficking or by inhibiting its association with binding partners. They have been largely employed as research tools and are emerging as therapeutic molecules for the treatment of human diseases such as viral pathologies, cancer and misfolding diseases. The fast-growing bio-market of recombinant antibodies provides intrabodies with enhanced binding specificity, stability, and solubility, together with lower immunogenicity, for their use in therapy (Biocca, abstract in Antibody Expression and Production Cell Engineering Volume 7, 2011, pp. 179-195).

In some embodiments, intrabodies have advantages over interfering RNA (iRNA); for example, iRNA has been shown to exert multiple non-specific effects, whereas intrabodies have been shown to have high specificity and affinity to target antigens. Furthermore, as proteins, intrabodies possess a much longer active half-life than iRNA. Thus, when the active half-life of the intracellular target molecule is long, gene silencing through iRNA may be slow to yield an effect, whereas the effects of intrabody expression can be almost instantaneous. Lastly, it is possible to design intrabodies to block certain binding interactions of a particular target molecule, while sparing others.

Intrabodies are often single chain variable fragments (scFvs) expressed from a recombinant nucleic acid molecule and engineered to be retained intracellularly (e.g., retained in the cytoplasm, endoplasmic reticulum, or periplasm). Intrabodies may be used, for example, to ablate the function of a protein to which the intrabody binds. The expression of intrabodies may also be regulated through the use of inducible promoters in the nucleic acid expression vector comprising the intrabody. Intrabodies may be produced for use in the viral genomes using methods known in the art, such as those disclosed and reviewed in: (Marasco et al., 1993 Proc. Natl. Acad. Sci. USA, 90: 7889-7893; Chen et al., 1994, Hum. Gene Ther. 5:595-601; Chen et al., 1994, Proc. Natl. Acad. Sci. USA, 91: 5932-5936; Maciejewski et al., 1995, Nature Med., 1: 667-673; Marasco, 1995, Immunotech, 1: 1-19; Mhashilkar, et al., 1995, EMBO J. 14: 1542-51; Chen et al., 1996, Hum. Gene Therap., 7: 1515-1525; Marasco, Gene Ther. 4:11-15, 1997; Rondon and Marasco, 1997, Annu. Rev. Microbiol. 51:257-283; Cohen, et al., 1998, Oncogene 17:2445-56; Proba et al., 1998, J. Mol. Biol. 275:245-253; Cohen et al., 1998, Oncogene 17:2445-2456; Hassanzadeh, et al., 1998, FEBS Lett. 437:81-6; Richardson et al., 1998, Gene Ther. 5:635-44; Ohage and Steipe, 1999, J. Mol. Biol. 291:1119-1128; Ohage et al., 1999, J. Mol. Biol. 291:1129-1134; Wirtz and Steipe, 1999, Protein Sci. 8:2245-2250; Zhu et al., 1999, J. Immunol. Methods 231:207-222; Arafat et al., 2000, Cancer Gene Ther. 7:1250-6; der Maur et al., 2002, J. Biol. Chem. 277:45075-85; Mhashilkar et al., 2002, Gene Ther. 9:307-19; and Wheeler et al., 2003, FASEB J. 17: 1733-5; and references cited therein). In particular, a CCR5 intrabody has been produced by Steinberger et al., 2000, Proc. Natl. Acad. Sci. USA 97:805-810). See generally Marasco, WA, 1998, “Intrabodies: Basic Research and Clinical Gene Therapy Applications” Springer: New York; and for a review of scFvs, see Pluckthun in “The Pharmacology of Monoclonal Antibodies,” 1994, vol. 113, Rosenburg and Moore eds. Springer-Verlag, New York, pp. 269-315.

Sequences from donor antibodies may be used to develop intrabodies. Intrabodies are often recombinantly expressed as single domain fragments such as isolated VH and VL domains or as a single chain variable fragment (scFv) antibody within the cell. For example, intrabodies are often expressed as a single polypeptide to form a single chain antibody comprising the variable domains of the heavy and light chains joined by a flexible linker polypeptide. Intrabodies typically lack disulfide bonds and are capable of modulating the expression or activity of target genes through their specific binding activity. Single chain antibodies can also be expressed as a single chain variable region fragment joined to the light chain constant region.

As is known in the art, an intrabody can be engineered into recombinant polynucleotide vectors to encode sub-cellular trafficking signals at its N or C terminus to allow expression at high concentrations in the sub-cellular compartments where a target protein is located. For example, intrabodies targeted to the endoplasmic reticulum (ER) are engineered to incorporate a leader peptide and, optionally, a C-terminal ER retention signal, such as the KDEL amino acid motif (SEQ ID NO: 4545). Intrabodies intended to exert activity in the nucleus are engineered to include a nuclear localization signal. Lipid moieties are joined to intrabodies in order to tether the intrabody to the cytosolic side of the plasma membrane. Intrabodies can also be targeted to exert function in the cytosol. For example, cytosolic intrabodies are used to sequester factors within the cytosol, thereby preventing them from being transported to their natural cellular destination.

There are certain technical challenges with intrabody expression. In particular, protein conformational folding and structural stability of the newly synthesized intrabody within the cell is affected by reducing conditions of the intracellular environment.

Intrabodies may be promising therapeutic agents for the treatment of misfolding diseases, including tauopathies, prion diseases, Alzheimer's, Parkinson's, and Huntington's, because of their virtually infinite ability to specifically recognize the different conformations of a protein, including pathological isoforms, and because they can be targeted to the potential sites of aggregation (both intra- and extracellular sites). These molecules can work as neutralizing agents against amyloidogenic proteins by preventing their aggregation, and/or as molecular shunters of intracellular traffic by rerouting the protein from its potential aggregation site (Cardinale, and Biocca, Curr. Mol. Med. 2008, 8:2-11).

### Maxibody

In some embodiments, the payloads encode a maxibody (bivalent scFv fused to the amino terminus of the Fc (CH2-CH3 domains) of IgG.

### Exemplary Payload Antibodies

In some embodiments, the payload region of the AAV particle comprises one or more nucleic acid sequences encoding anti-tau antibody molecules, e.g., an anti-tau antibody molecule described herein. Human tau can have, for example, the following sequence:

(isoform 2; SEQ ID NO: 9200)
MAEPRQEFEVMEDHAGTYGLGDRKDQGGYTMHQDQEGDTDAGLKES
PLQTPTEDGSEEPGSETSDAKSTPTAEDVTAPLVDEGAPGKQAAA
QPHTEIPEGTTAEEAGIGDTPSLEDEAAGHVTQARMVSKSKDGTG
SDDKKAKGADGKIKIATPRGAAPPGQKGQANATRIPAKTPPAPKT
PPSSGEPPKSGDRSGYSSPGSPGTPGSRSRTPSLPTPPTREPKKV
AVVRTPPKSPSSAKSRLQTAPVPMPDLKNVKSKIGSTENLKHQPG
GGKVQIINKKLDLSNVQSKCGSKDNIKHVPGGGSVQIVYKPVDLS
KVTSKCGSLGNIHHKPGGGQVEVKSEKLDFKDRVQSKIGSLDNIT
HVPGGGNKKIETHKLTFRENAKAKTDHGAEIVYKSPVVSGDTSPR
HLSNVSSIGSIDMVDSPQLATLADEVSASLAKQGL

In some embodiments, the anti-tau antibody molecule is specific for one or more epitopes or phosphorylated Ser and/or Thr residues in tau (e.g., human tau), for example, in the N-terminal, mid-, and/or C-terminal domains of tau. In some embodiments, the anti-tau antibody molecule is specific for one or more epitopes or phosphorylated Ser and/or Thr residues in the N-terminal domain of tau (such as the N-terminal domain N-terminal to the 1N domain, or residues 15-25 or 1 to about 50 of the human tau protein). In some embodiments, the anti-tau antibody molecule is specific for one or more epitopes or phosphorylated Ser and/or Thr residues in the mid-domain of tau (such as the mid-domain comprising, consisting essentially, or consisting of residues between the 2N and R1 domains, or residues above 95 to about 250 of the human tau protein). In some embodiments, the anti-tau antibody molecule is specific for one or more epitopes or phosphorylated Ser and/or Thr residues in the C-terminal domain of tau (such as the C-terminal domain comprising, consisting essentially, or consisting of residues C-terminal to the R4 domain, or residues about 370 to 441 of the human tau protein).

Exemplary and non-limiting N-terminal domain epitopes include amino acid residues (a) 9-18, (b) 15-25, (c) 25-30, and (d) 15-30 of human tau (e.g., numbered according to SEQ ID NO: 9200). Accordingly, in some embodiments, the anti-tau antibody molecule described herein binds to all or a portion of (e.g., one or more residues within) amino acid residues (a) 9-18, (b) 15-25, (c) 25-30, and/or (d) 15-30 of human tau (e.g., numbered according to SEQ ID NO: 9200).

Exemplary and non-limiting mid-domain epitopes include amino acid residues (a) 125-131, (b) 204-222, (c) 234-246, (d) 234-259, and (e) 235-246 of human tau (e.g., numbered according to SEQ ID NO: 9200). Accordingly, in some embodiments, the anti-tau antibody molecule described herein binds to all or a portion of (e.g., one or more residues within) amino acid residues (a) 125-131, (b) 204-222, (c) 234-246, (d) 234-259, and/or (e) 235-246 of human tau (e.g., numbered according to SEQ ID NO: 9200).

Exemplary and non-limiting mid-domain epitopes include pT181, pS199, pS202, pT205, pT212, pS214, pT217, pT231, pS234, pS235, pS258, and pS259 of human tau (e.g., numbered according to SEQ ID NO: 9200).

Exemplary and non-limiting C-terminal domain epitopes include amino acid residues (a) 387-408 and (b) 409-436 of human tau (e.g., numbered according to SEQ ID NO: 9200). Accordingly, in some embodiments, the anti-tau antibody molecule described herein binds to all or a portion of (e.g., one or more residues within) amino acid residues (a) 387-408 and/or (b) 409-436 of human tau (e.g., numbered according to SEQ ID NO: 9200).

Exemplary and non-limiting C-terminal domain epitopes include pS396, pS404, and/or pS409 of human tau (e.g., numbered according to SEQ ID NO: 9200).

In certain embodiments, the antibody molecule recognizes a 3D conformation epitope with residues not contiguous on the primary sequence.

In some embodiments, the anti-tau antibody molecule binds to the N-terminal domain of human tau, e.g., an N-terminal domain epitope on human tau. In some embodiments, the N-terminal domain of human tau spans amino acid residues 1-103 of human tau. Exemplary and non-limiting N-terminal domain-binding antibodies include IPN002 and those disclosed in WO2018/106781, WO2015/200806, WO2014/165271, WO2017/191559, WO2014/028777, WO2015/197820, WO2019/110571, and WO2017/191561, the contents of all of which are herein incorporated by reference in their entirety, and antibodies that either bind to the same epitope as or compete for binding with the aforementioned antibodies.

In some embodiments, the anti-tau antibody molecule binds to the mid-domain of human tau, e.g., a mid-domain epitope on human tau. In some embodiments, the mid-domain of human tau spans amino acid residues 103-244 of human tau, and optionally may include the microtubule binding region, which spans amino acid residues 244-369 of human tau. Exemplary and non-limiting mid-domain-binding antibodies include AT8, PT3, UCB, PT76, and those disclosed in WO2017/191561, WO2014/028777, WO2015/120364, WO2015/122922, WO2004/016655, WO2014/152157, WO2015/197823, WO2018/170351, WO2019/171258, WO2019/171259, WO2021/205359, WO2017/005732, WO2017/005732, WO2019/094595, WO2022/144406, WO2012/149365, WO2017/005734, U.S. Pat. No. 7,176,290, WO2013/041962, WO2016/079597, WO2020/097561, WO2020/180819, WO2015/197820, WO2018/178077, WO2019/077500, and WO2012/149365, and antibodies that either bind to the same epitope as or compete for binding with the aforementioned antibodies.

In some embodiments, the anti-tau antibody molecule binds to the microtubule-binding region of human tau, e.g., a microtubule-binding region epitope on human tau. In some embodiments, the microtubule binding region spans amino acid residues 244-369 of human tau. Exemplary and non-limiting microtubule-binding region binding antibodies include WO2017/005734, U.S. Pat. No. 7,176,290, WO2013/041962, WO2016/079597, WO2020/097561, WO2020/180819, WO2015/197820, WO2018/178077, WO2019/077500, and WO2012/149365, and antibodies that either bind to the same epitope as or compete for binding with the aforementioned antibodies.

In some embodiments, the anti-tau antibody molecule binds to the C-terminal domain of human tau, e.g., a C-terminal domain epitope on human tau. In some embodiments, the N-terminal domain of human tau spans amino acid residues 1-103 of human tau. Exemplary and non-limiting C-terminal domain-binding antibodies include PHF1, C10.2, and those disclosed in US20070280935, WO2010/142423, WO2015/091656, WO2015/114538, WO2015/197735, WO2016/207245, WO2012/045882, WO2013/050567, WO2013/151762, WO2012/106363, WO2013/180238, WO2014/096321, WO2014/150877, WO2017/009308, WO2018/127519, WO2017/191560, U.S. Ser. No. 10/556,950, WO2020/201828, and WO2021/262791, and antibodies that either bind to the same epitope as or compete for binding with the aforementioned antibodies.

In some embodiments, the anti-tau antibody molecule binds to a region that overlaps with the N-terminal domain and the mid-domain of human tau. Exemplary and non-limiting antibodies which bind to a region that overlaps with the N-terminal domain and the mid-domain of human tau include those disclosed in WO2017/191561 and WO2014/028777, and antibodies that either bind to the same epitope as or compete for the aforementioned antibodies.

In some embodiments, the anti-tau antibody molecule binds to a region that overlaps with the mid-domain and the microtubule-binding region of human tau. Exemplary and non-limiting antibodies which bind to a region that overlaps with the mid-domain and the microtubule-binding region of human tau include those described in WO2017/005734, and antibodies that either bind to the same epitope as or compete for binding with the aforementioned antibodies.

In some embodiments, the anti-tau antibody molecule binds to a region that overlaps with the mid-domain and the C-terminal domain of human tau. Exemplary and non-limiting antibodies which bind to a region that overlaps with the mid-domain and the C-terminal domain of human tau include those described in WO2012/149365, and antibodies that either bind to the same epitope as or compete for binding with the aforementioned antibodies.

In some embodiments, the anti-tau antibody molecule binds to all or a portion of (e.g., one or more residues within) amino acid residues (a) 32-49, (b) 55-76, (c) 57-72, (d) 159-194, (e) 175-191, (f) 185-200, (g) 219-247, (h) 223-238, (i) 381-426, (j) 383-400, (k) 409-436, and/or (1) 413-430 of human tau (SEQ ID NO: 9200). In some embodiments, the anti-tau antibody molecule competes for binding with an anti-tau antibody molecule which binds to all or a portion of (e.g., one or more residues within) amino acid residues (a) 32-49, (b) 55-76, (c) 57-72, (d) 159-194, (e) 175-191, (f) 185-200, (g) 219-247, (h) 223-238, (i) 381-426, (j) 383-400, (k) 409-436, and/or (1) 413-430 of human tau (SEQ ID NO: 9200). In some embodiments, the anti-tau antibody molecule binds to the same epitope as, substantially the same epitope as, an epitope that overlaps with, or an epitope that substantially overlaps with, an anti-tau antibody molecule which binds to all or a portion of (e.g., one or more residues within) amino acid residues (a) 32-49, (b) 55-76, (c) 57-72, (d) 159-194, (e) 175-191, (f) 185-200, (g) 219-247, (h) 223-238, (i) 381-426, 0) 383-400, (k) 409-436, and/or (1) 413-430 of human tau (SEQ ID NO: 9200).

Non-limiting examples of anti-tau antibody molecules suitable for use as a payload in the AAV particles described herein include, for example, one or more of the antibody molecules listed in Tables 7-16. As another non-limiting example, the payload region of the AAV particle comprises a nucleotide sequence encoding an antibody molecule which may comprise one or more of the HCDRs, VH sequences, or heavy chain sequences listed in Tables 7-16. As a further non-limiting example, the payload region of the AAV particle comprises a nucleotide sequence encoding an antibody molecule which may comprise one or more of the LCDRs, VL sequences, or light chain sequences listed in Tables 7-16. In yet a further non-limiting example, the payload region of the AAV particle comprises a nucleotide sequence encoding an antibody molecule which may comprise one or more of the HCDRs, VH sequences, or heavy chain sequences listed in Tables 7-16, and one or more of the LCDRs, VL sequences, or light chain sequences listed in Tables 7-16.

In some embodiments, the payload region may also comprise a linker between the VH and VL sequences, or between the heavy and light chain sequences. The linker may be a sequence known in the art or a linker described in Table 19.

In some embodiments, the payload region of the AAV particle comprises a nucleic acid sequence encoding a polypeptide comprising a VH sequence and a VL sequence, or a heavy chain sequence and a light chain sequence, from Tables 7-16, where the VH sequence or heavy chain sequence is from a different antibody than the VL sequence or light chain sequence.

In some embodiments, the payload region comprises, in the 5′ to 3′ direction, an antibody light chain sequence, a linker region, and a heavy chain sequence, or a VL sequence, a linker region, and a VH sequence. In another embodiment, the linker is not used.

In some embodiments, the payload region comprises a nucleic acid sequence encoding, in the 5′ to 3′ direction, an antibody light chain sequence, a linker region, and a heavy chain sequence, or a VL sequence, a linker region, and a VH sequence.

In some embodiments, the payload region comprises, in the 5′ to 3′ direction, an antibody heavy chain sequence, a linker region, and a light chain sequence, or a VH sequence, a linker region, and a VL sequence. In another embodiment, the linker is not used.

In some embodiments, the payload region comprises a nucleic acid sequence encoding, in the 5′ to 3′ direction, an antibody heavy chain sequence, a linker region, and a light chain sequence, or a VH sequence, a linker region, and a VL sequence.

In some embodiments, the payload region comprises a nucleic acid sequence encoding a single heavy chain. As a non-limiting example, the heavy chain is an amino acid sequence described in Tables 7-16.

In some embodiments, the payload region of the AAV particle comprises one or more nucleic acid sequences encoding one or more of the antibody molecules described herein (e.g., an antibody molecule listed in any one of Tables 7-16).

Shown in Tables 7-16 is a listing of antibody molecules and their polynucleotides and/or polypeptides sequences. These sequences may be encoded by or included in the AAV particles of the present disclosure. Variants or fragments of the antibody sequences described in Tables 7-16 may be utilized in the AAV particles of the present disclosure.

In some embodiments, the payload region of the AAV particle comprises a nucleic acid sequence encoding an antibody molecule with at least 50% identity, for example, at least 60%, at least 70%, at least 80%, at least 85%, at least 90%, at least 91%, at least 92%, at least 93%, at least 94%, at least 95%, at least 96%, at least 97%, at least 98%, at least 99%, or 100% identity to the amino acid sequence of one or more of the antibody molecules described herein (e.g., an antibody molecule listed in Tables 7-16).

In some embodiments, the payload region of the AAV particle comprises a nucleic acid sequence with at least 50% identity, for example, at least 60%, at least 70%, at least 80%, at least 85%, at least 90%, at least 91%, at least 92%, at least 93%, at least 94%, at least 95%, at least 96%, at least 97%, at least 98%, at least 99%, or 100% identity to one or more of the nucleotide sequences listed in Tables 7-16.

In some embodiments, the CDR region of the encoded antibody molecule may have at least 50% identity, for example, at least 60%, at least 70%, at least 80%, at least 85%, at least 90%, at least 91%, at least 92%, at least 93%, at least 94%, at least 95%, at least 96%, at least 97%, at least 98%, at least 99%, or 100% identity to at least one or more of the VCDR1, VCDR2, and/or VCDR3, and/or LCDR1, LCDR2, and/or LCDR3 (e.g., one, two, three, four, five, or all six CDRs) sequences of one or more of the antibody molecules described herein (e.g., an antibody molecule listed in Tables 7-16).

In some embodiments, the VH sequence and/or VL sequence of the antibody molecule has at least 50% identity, for example, at least 60%, at least 70%, at least 80%, at least 85%, at least 90%, at least 91%, at least 92%, at least 93%, at least 94%, at least 95%, at least 96%, at least 97%, at least 98%, at least 99%, or 100% identity to the amino acid sequence of the VH and/or VL sequences of one or more of the antibody molecules described herein (e.g., an antibody molecule listed in Tables 7-16).

In some embodiments, the heavy chain and/or light chain of the encoded antibody molecule has at least 50% identity, for example, at least 60%, at least 70%, at least 80%, at least 85%, at least 90%, at least 91%, at least 92%, at least 93%, at least 94%, at least 95%, at least 96%, at least 97%, at least 98%, at least 99%, or 100% identity to the amino acid sequence of the heavy chain and/or light chain of one or more of the antibody molecules described herein (e.g., an antibody molecule listed in Tables 7-16).

TABLE 7
Tau-associated disease antibodies

| | | Type | | | | SEQ |
|---|---|---|---|---|---|---|
| Ab | Compo- | (PRT/ | | | | ID |
| ID | nent | DNA) | Description | Reference | Length | NO |
| Ab1 | VH | DNA | IPN002 | | 57 | 1740 |
| | Signal | | (VH | | | |
| | | | Signal) | | | |
| Ab2 | VH | DNA | PHF1 | | 57 | 1741 |
| | Signal | | (VH Signal) | | | |
| Ab3 | VH | DNA | VH | | 969 | 1742 |
| | Constant | | (constant- | | | |
| | | | 969) | | | |
| Ab4 | VH | DNA | VH | | 972 | 1743 |
| | Constant | | (constant- | | | |
| | | | 972) | | | |
| Ab5 | VH CDR | PRT | 7295-M6 HCDR | WO2015122922; | 10 | 1744 |
| | | | | SEQ ID NO: 12 | | |
| | | | | and 20 | | |
| Ab6 | VH CDR | PRT | 7295-M6 HCDR | WO2015122922; | 16 | 1745 |
| | | | | SEQ ID NO: 13 | | |
| | | | | and 21 | | |
| Ab7 | VH CDR | PRT | 7295-M6 HCDR | WO2015122922; | 4 | 1746 |
| | | | | SEQ ID NO: 14 | | |
| | | | | and 22 | | |
| Ab8 | VH CDR | PRT | 7297-2M1 HCDR | WO2015122922; | 16 | 1747 |
| | | | | SEQ ID NO: 69 | | |
| Ab9 | VH CDR | PRT | 7298-M1 HCDR | WO2015122922; | 11 | 1748 |
| | | | | SEQ ID NO: 28 | | |
| Ab10 | VH CDR | PRT | 7298-M1 HCDR | WO2015122922; | 16 | 1749 |
| | | | | SEQ ID NO: 29 | | |
| Ab11 | VH CDR | PRT | 7298-M1 HCDR | WO2015122922; | 4 | 1750 |
| | | | | SEQ ID NO: 30 | | |
| Ab12 | VH CDR | PRT | 7298-M2 HCDR | WO2015122922; | 10 | 1751 |
| | | | | SEQ ID NO: 36 | | |
| Ab13 | VH CDR | PRT | 7298-M2 HCDR | WO2015122922; | 14 | 1752 |
| | | | | SEQ ID NO: 37 | | |
| Ab14 | VH CDR | PRT | 7298-M2 HCDR | WO2015122922; | 8 | 1753 |
| | | | | SEQ ID NO: 38 | | |
| Ab15 | VH CDR | PRT | 7299-M2 HCDR | WO2015122922; | 10 | 1754 |
| | | | | SEQ ID NO: 44 | | |
| | | | | and 60 | | |
| Ab16 | VH CDR | PRT | 7299-M2 HCDR | WO2015122922; | 16 | 1755 |
| | | | | SEQ ID NO: 45 | | |
| Ab17 | VH CDR | PRT | 7299-M2 HCDR | WO2015122922; | 4 | 1756 |
| | | | | SEQ ID NO: 46 | | |
| | | | | and 62 | | |
| Ab18 | VH CDR | PRT | 7299-M5 HCDR | WO2015122922; | 10 | 1757 |
| | | | | SEQ ID NO: 52 | | |
| | | | | and 68 | | |
| Ab19 | VH CDR | PRT | 7299-M5 HCDR | WO2015122922; | 16 | 1758 |
| | | | | SEQ ID NO: 53 | | |
| Ab20 | VH CDR | PRT | 7299-M5 HCDR | WO2015122922; | 10 | 1759 |
| | | | | SEQ ID NO: 54 | | |
| | | | | and 70 | | |
| Ab21 | VH CDR | PRT | 7299-M9 HCDR | WO2015122922; | 15 | 1760 |
| | | | | SEQ ID NO: 61 | | |
| Ab22 | VH CDR | PRT | C10.2 HCDR1 | WO2017009308; | 13 | 1761 |
| | | | | SEQ ID NO: 12, 20 | | |
| | | | | and 28 | | |
| Ab23 | VH CDR | PRT | C10.2 HCDR2 | WO2017009308; | 16 | 1762 |
| | | | | SEQ ID NO: 13 | | |
| | | | | and 29 | | |
| Ab24 | VH CDR | PRT | C10.2 HCDR3 | WO2017009308; | 6 | 1763 |
| | | | | SEQ ID NO: 14 | | |
| | | | | and 30 | | |
| Ab25 | VH CDR | PRT | C5.2 HCDR2 | WO2017009308; | 16 | 1764 |
| | | | | SEQ ID NO: 21 | | |
| Ab26 | VH CDR | PRT | C5.2 HCDR3 | WO2017009308; | 6 | 1765 |
| | | | | SEQ ID NO: 22 | | |
| Ab27 | VH CDR | PRT | D1.2 HCDR1 | WO2017009308; | 13 | 1766 |
| | | | | SEQ ID NO: 4 | | |
| Ab28 | VH CDR | PRT | D1.2 HCDR2 | WO2017009308; | 16 | 1767 |
| | | | | SEQ ID NO: 5 | | |
| Ab29 | VH CDR | PRT | D1.2 HCDR3 | WO2017009308; | 6 | 1768 |
| | | | | SEQ ID NO: 6 | | |
| Ab30 | VH CDR | PRT | HCDR1 | U.S. Pat. No. 8,980,270; | 5 | 1769 |
| | | | | SEQ ID NO: 4 | | |
| Ab31 | VH CDR | PRT | HCDR1 | U.S. Pat. No. 8,980,270; | 5 | 1770 |
| | | | | SEQ ID NO: 10 | | |
| Ab32 | VH CDR | PRT | HCDR2 | U.S. Pat. No. 8,980,270; | 17 | 1771 |
| | | | | SEQ ID NO: 5 | | |
| Ab33 | VH CDR | PRT | HCDR2 | U.S. Pat. No. 8,980,270; | 17 | 1772 |
| | | | | SEQ ID NO: 11 | | |
| Ab34 | VH CDR | PRT | HCDR2 | WO2015081085; | 32 | 1773 |
| | | | | SEQ ID NO: 27 | | |
| Ab35 | VH CDR | PRT | HCDR3 | U.S. Pat. No. 8,980,270; | 8 | 1774 |
| | | | | SEQ ID NO: 6 | | |
| Ab36 | VH CDR | PRT | HCDR3 | U.S. Pat. No. 8,980,270; | 8 | 1775 |
| | | | | SEQ ID NO: 12 | | |
| Ab37 | VH CDR | PRT | HCDR3 | WO2015081085; | 11 | 1776 |
| | | | | SEQ ID NO: 44 | | |
| Ab38 | VH CDR | PRT | IPN002 HCDR1 | WO2014028777; | 14 | 1777 |
| | | | | SEQ ID NO: 84 | | |
| Ab39 | VH CDR | PRT | IPN002 HCDR2 | WO2014028777; | 32 | 1778 |
| | | | | SEQ ID NO: 85 | | |
| Ab40 | VH CDR | PRT | IPN002 HCDR3 | WO2014028777; | 11 | 1779 |
| | | | | SEQ ID NO: 86 | | |
| Ab41 | VH CDR | PRT | MC1 LCDR1 | WO2016137811; | 13 | 1780 |
| | | | | SEQ ID NO: 6 | | |
| Ab42 | VH CDR | PRT | MC1 LCDR2 | WO2016137811; | 18 | 1781 |
| | | | | SEQ ID NO: 7 | | |
| Ab43 | VH CDR | PRT | MC1 LCDR3 | WO2016137811; | 9 | 1782 |
| | | | | SEQ ID NO: 8 | | |
| Ab44 | VH CDR | PRT | PT1 HCDR1 | U.S. Pat. No. 9,371,376; | 7 | 1783 |
| | | | (Chothia) | SEQ ID NO: 19 | | |
| Ab45 | VH CDR | PRT | PT1 HCDR1 | U.S. Pat. No. 9,371,376; | 5 | 1784 |
| | | | (Kabat) | SEQ ID NO: 7 | | |
| Ab46 | VH CDR | PRT | PT1 HCDR2 | U.S. Pat. No. 9,371,376; | 6 | 1785 |
| | | | (Chothia) | SEQ ID NO: 20 | | |
| Ab47 | VH CDR | PRT | PT1 HCDR2 | U.S. Pat. No. 9,371,376; | 17 | 1786 |
| | | | (Kabat) | SEQ ID NO: 8 | | |
| Ab48 | VH CDR | PRT | PT1 HCDR3 | U.S. Pat. No. 9,371,376; | 9 | 1787 |
| | | | (Chothia) | SEQ ID NO: 21 | | |
| Ab49 | VH CDR | PRT | PT1 HCDR3 | U.S. Pat. No. 9,371,376; | 10 | 1788 |
| | | | (Kabat) | SEQ ID NO: 9 | | |
| Ab50 | VH CDR | PRT | PT3 HCDR1 | U.S. Pat. No. 9,371,376; | 7 | 1789 |
| | | | (Chothia) | SEQ ID NO: 25 | | |
| Ab51 | VH CDR | PRT | PT3 HCDR1 | U.S. Pat. No. 9,371,376; | 5 | 1790 |
| | | | (Kabat) | SEQ ID NO: 13 | | |
| Ab52 | VH CDR | PRT | PT3 HCDR2 | U.S. Pat. No. 9,371,376; | 5 | 1791 |
| | | | (Chothia) | SEQ ID NO: 26 | | |
| Ab53 | VH CDR | PRT | PT3 HCDR2 | U.S. Pat. No. 9,371,376; | 16 | 1792 |
| | | | (Kabat) | SEQ ID NO: 14 | | |
| Ab54 | VH CDR | PRT | PT3 HCDR3 | U.S. Pat. No. 9,371,376; | 9 | 1793 |
| | | | (Chothia) | SEQ ID NO: 27 | | |
| Ab55 | VH CDR | PRT | PT3 HCDR3 | U.S. Pat. No. 9,371,376; | 10 | 1794 |
| | | | (Kabat) | SEQ ID NO: 15 | | |
| Ab56 | VH | PRT | 7295-M6 VH | WO2015122922; | 125 | 1795 |
| | | | | SEQ ID NO: 16 | | |
| | | | | and 24 | | |
| Ab57 | VH | PRT | 7297-2M1 VH | WO2015122922; | 131 | 1796 |
| | | | | SEQ ID NO: 72 | | |
| Ab58 | VH | PRT | 7298-M1 VH | WO2015122922; | 127 | 1797 |
| | | | | SEQ ID NO: 32 | | |
| Ab59 | VH | PRT | 7298-M2 VH | WO2015122922; | 128 | 1798 |
| | | | | SEQ ID NO: 40 | | |
| Ab60 | VH | PRT | 7299-M2 VH | WO2015122922; | 125 | 1799 |
| | | | | SEQ ID NO: 48 | | |
| Ab61 | VH | PRT | 7299-M5 VH | WO2015122922; | 131 | 1800 |
| | | | | SEQ ID NO: 56 | | |
| Ab62 | VH | PRT | 7299-M9 VH | WO2015122922; | 125 | 1801 |
| | | | | SEQ ID NO: 64 | | |
| Ab63 | VH | PRT | C10.2 VH | WO2017009308; | 439 | 1802 |
| | | | | SEQ ID NO: 16 | | |
| Ab64 | VH | PRT | C10.2 VH − 115 | | 115 | 1803 |
| Ab65 | VH | DNA | C10.2 VH | | 345 | 1804 |
| | | | Bioinformatics | | | |
| Ab66 | VH | DNA | C10.2 VH | | 345 | 1805 |
| | | | EMBOSS | | | |
| Ab67 | VH | DNA | C10.2 VH | | 345 | 1806 |
| | | | GeneInfinity | | | |
| Ab68 | VH | DNA | C10.2 VH | | 345 | 1807 |
| | | | GregThatcher | | | |
| Ab69 | VH | DNA | C10.2 VH IDT | | 345 | 1808 |
| Ab70 | VH | DNA | C10.2 VH | | 345 | 1809 |
| | | | InSilico | | | |
| Ab71 | VH | DNA | C10.2 VH MolBio | | 345 | 1810 |
| Ab72 | VH | DNA | C10.2 VH N2P | | 345 | 1811 |
| Ab73 | VH | DNA | C10.2 VH | | 345 | 1812 |
| | | | Snapgene | | | |
| Ab74 | VH | DNA | C10.2 VH Vector | | 345 | 1813 |
| | | | NTI | | | |
| Ab75 | VH | PRT | C5.2 VH | WO2017009308; | 439 | 1814 |
| | | | | SEQ ID NO: 24 | | |
| Ab76 | VH | PRT | C8.3 VH | WO2017009308; | 439 | 1815 |
| | | | | SEQ ID NO: 32 | | |
| Ab77 | VH | PRT | D1.2 VH | WO2017009308; | 451 | 1816 |
| | | | | SEQ ID NO: 8 | | |
| Ab78 | VH | PRT | hC10.2 VH | WO2017009308; | 444 | 1817 |
| | | | | SEQ ID NO: 35 | | |
| Ab79 | VH | DNA | IPN001 VH − 351 | U.S. Pat. No. 8,980,270; | 351 | 1818 |
| | | | | SEQ ID NO: 18 | | |
| Ab80 | VH | PRT | IPN001 VH − 117 | U.S. Pat. No. 8,980,270; | 117 | 1819 |
| | | | | SEQ ID NO: 14 | | |
| Ab81 | VH | DNA | IPN002 VH − 1377 | | 1377 | 1820 |
| Ab82 | VH | DNA | IPN002 VH − 351 | U.S. Pat. No. 8,980,270; | 351 | 1821 |
| | | | | SEQ ID NO: 20 | | |
| Ab83 | VH | PRT | IPN002 VH − 117 | U.S. Pat. No. 8,980,270; | 117 | 1822 |
| | | | | SEQ ID NO: 16 | | |
| Ab84 | VH | PRT | IPN002 VH − 30 | WO2014028777; | 30 | 1823 |
| | | | | SEQ ID NO: 83 | | |
| Ab85 | VH | DNA | IPN002 VH | U.S. Pat. No. 8,980,270; | 351 | 1824 |
| | | | variant 1 | SEQ ID NO: 28 | | |
| Ab86 | VH | PRT | IPN002 VH | U.S. Pat. No. 8,980,270; | 117 | 1825 |
| | | | variant 1 | SEQ ID NO: 36 | | |
| Ab87 | VH | DNA | IPN002 VH | U.S. Pat. No. 8,980,270; | 351 | 1826 |
| | | | variant 2 | SEQ ID NO: 29 | | |
| Ab88 | VH | PRT | IPN002 VH | U.S. Pat. No. 8,980,270; | 117 | 1827 |
| | | | variant 2 | SEQ ID NO: 37 | | |
| Ab89 | VH | DNA | IPN002 VH | U.S. Pat. No. 8,980,270; | 351 | 1828 |
| | | | variant 3 | SEQ ID NO: 30 | | |
| Ab90 | VH | PRT | IPN002 VH | U.S. Pat. No. 8,980,270; | 117 | 1829 |
| | | | variant 3 | SEQ ID NO: 38 | | |
| Ab91 | VH | DNA | IPN002 VH | U.S. Pat. No. 8,980,270; | 351 | 1830 |
| | | | variant 4 | SEQ ID NO: 31 | | |
| Ab92 | VH | PRT | IPN002 VH | U.S. Pat. No. 8,980,270; | 117 | 1831 |
| | | | variant 4 | SEQ ID NO: 39 | | |
| Ab93 | VH | DNA | MC1 VH − 1374 | | 1374 | 1832 |
| Ab94 | VH | DNA | MC1 VH − 1326 | WO2016137811; | 1326 | 1833 |
| | | | | SEQ ID NO: 12 | | |
| Ab95 | VH | PRT | MC1 VH − 458 | | 458 | 1834 |
| Ab96 | VH | PRT | MC1 VH − 442 | WO2016137811; | 442 | 1835 |
| | | | | SEQ ID NO: 2 | | |
| Ab97 | VH | PRT | MC1 VH − 116 | WO2016137811; | 116 | 1836 |
| | | | | SEQ ID NO: 10 | | |
| Ab98 | VH | DNA | PHF1 VH − 1320 | | 1320 | 1837 |
| Ab99 | VH | DNA | PHF1 VH − 1377 | | 1377 | 1838 |
| Ab100 | VH | DNA | PHF1 VH − 351 | | 351 | 1839 |
| Ab101 | VH | DNA | PHF1 VH − 699 | | 699 | 1840 |
| Ab102 | VH | DNA | PHF1 VH − 717 | | 717 | 1841 |
| Ab103 | VH | DNA | PHF1 VH − 744 | | 744 | 1842 |
| Ab104 | VH | DNA | PHF1 VH − 753 | | 753 | 1843 |
| Ab105 | VH | PRT | PHF1 VH − 459 | | 459 | 1844 |
| Ab106 | VH | DNA | PHF1 VH | | 1383 | 1845 |
| Ab107 | VH | DNA | PT1 VH − 357 | U.S. Pat. No. 9,371,376; | 357 | 1846 |
| | | | | SEQ ID NO: 31 | | |
| Ab108 | VH | PRT | PT1 VH − 119 | U.S. Pat. No. 9,371,376; | 119 | 1847 |
| | | | | SEQ ID NO: 35 | | |
| Ab109 | VH | DNA | PT3 VH − 354 | U.S. Pat. No. 9,371,376; | 354 | 1848 |
| | | | | SEQ ID NO: 33 | | |
| Ab110 | VH | PRT | PT3 VH − 118 | U.S. Pat. No. 9,371,376; | 118 | 1849 |
| | | | | SEQ ID NO: 37 | | |
| Ab111 | VH | DNA | PT3 VH | | 354 | 1850 |
| | | | Bioinformatics | | | |
| Ab112 | VH | DNA | PT3 VH | | 354 | 1851 |
| | | | Bioinformatics 2 | | | |
| Ab113 | VH | DNA | PT3 VH EMBOSS | | 354 | 1852 |
| Ab114 | VH | DNA | PT3 VH | | 354 | 1853 |
| | | | Geneinfinity | | | |
| Ab115 | VH | DNA | PT3 VH | | 354 | 1854 |
| | | | Genescript | | | |
| Ab116 | VH | DNA | PT3 VH IDT | | 354 | 1855 |
| Ab117 | VH | DNA | PT3 VH NUS | | 354 | 1856 |
| Ab118 | VH | DNA | PT3 VH NUS2 | | 354 | 1857 |
| Ab119 | VH | DNA | PT3 VH NUS3 | | 354 | 1858 |
| Ab120 | VH | DNA | PT3 VH | | 354 | 1859 |
| | | | Snapgene | | | |
| Ab121 | VH | PRT | VH | WO2015081085; | 30 | 1860 |
| | | | | SEQ ID NO: 25 | | |
| Ab122 | VL | DNA | IPN002 (VL | | 57 | 1861 |
| | Signal | | Signal) | | | |
| Ab123 | VL | DNA | PHF1 (VL Signal) | | 57 | 1862 |
| | Signal | | | | | |
| Ab124 | VL | DNA | PHF1 (VL Signal | | 72 | 1863 |
| | Signal | | 72) | | | |
| Ab125 | VL | DNA | VL (constant) | | 321 | 1864 |
| | Constant | | | | | |
| Ab126 | VL CDR | PRT | 7295-M6 LCDR | WO2015122922; | 16 | 1865 |
| | | | | SEQ ID NO: 9 | | |
| | | | | and 17 | | |
| Ab127 | VL CDR | PRT | 7295-M6 LCDR | WO2015122922; | 9 | 1866 |
| | | | | SEQ ID NO: 11 | | |
| | | | | and 19 | | |
| Ab128 | VL CDR | PRT | 7297-2M1 LCDR | WO2015122922; | 11 | 1867 |
| | | | | SEQ ID NO: 65 | | |
| Ab129 | VL CDR | PRT | 7297-2M1 LCDR | WO2015122922; | 7 | 1868 |
| | | | | SEQ ID NO: 66 | | |
| Ab130 | VL CDR | PRT | 7297-2M1 LCDR | WO2015122922; | 8 | 1869 |
| | | | | SEQ ID NO: 67 | | |
| Ab131 | VL CDR | PRT | 7298-M1 LCDR | WO2015122922; | 15 | 1870 |
| | | | | SEQ ID NO: 25 | | |
| | | | | and 33 | | |
| Ab132 | VL CDR | PRT | 7298-M1 LCDR | WO2015122922; | 7 | 1871 |
| | | | | SEQ ID NO: 26 | | |
| | | | | and 34 | | |
| Ab133 | VL CDR | PRT | 7298-M1 LCDR | WO2015122922; | 9 | 1872 |
| | | | | SEQ ID NO: 27 | | |
| | | | | and 35 | | |
| Ab134 | VL CDR | PRT | 7299-M2 LCDR | WO2015122922; | 16 | 1873 |
| | | | | SEQ ID NO: 41 | | |
| Ab135 | VL CDR | PRT | 7299-M2 LCDR | WO2015122922; | 7 | 1874 |
| | | | | SEQ ID NO: 42 | | |
| Ab136 | VL CDR | PRT | 7299-M2 LCDR | WO2015122922; | 9 | 1875 |
| | | | | SEQ ID NO: 43 | | |
| Ab137 | VL CDR | PRT | 7299-M5 LCDR | WO2015122922; | 16 | 1876 |
| | | | | SEQ ID NO: 49 | | |
| | | | | and 57 | | |
| Ab138 | VL CDR | PRT | 7299-M5 LCDR | WO2015122922; | 7 | 1877 |
| | | | | SEQ ID NO: 50 | | |
| | | | | and 58 | | |
| Ab139 | VL CDR | PRT | 7299-M5 LCDR | WO2015122922; | 9 | 1878 |
| | | | | SEQ ID NO: 51 | | |
| Ab140 | VL CDR | PRT | 7299-M9 LCDR | WO2015122922; | 8 | 1879 |
| | | | | SEQ ID NO: 59 | | |
| Ab141 | VL CDR | PRT | C10.2 LCDR1 | WO2017009308; | 11 | 1880 |
| | | | | SEQ ID NO: 9 | | |
| | | | | and 25 | | |
| Ab142 | VL CDR | PRT | C10.2 LCDR2 | WO2017009308; | 7 | 1881 |
| | | | | SEQ ID NO: 10 | | |
| | | | | and 18 | | |
| Ab143 | VL CDR | PRT | C10.2 LCDR3 | WO2017009308; | 7 | 1882 |
| | | | | SEQ ID NO: 11 | | |
| | | | | and 19 | | |
| Ab144 | VL CDR | PRT | C5.2 LCDR1 | WO2017009308; | 11 | 1883 |
| | | | | SEQ ID NO: 17 | | |
| Ab145 | VL CDR | PRT | C8.3 LCDR2 | WO2017009308; | 7 | 1884 |
| | | | | SEQ ID NO: 26 | | |
| Ab146 | VL CDR | PRT | C8.3 LCDR3 | WO2017009308; | 7 | 1885 |
| | | | | SEQ ID NO: 27 | | |
| Ab147 | VL CDR | PRT | D1.2 LCDR1 | WO2017009308; | 16 | 1886 |
| | | | | SEQ ID NO: 1 | | |
| Ab148 | VL CDR | PRT | D1.2 LCDR2 | WO2017009308; | 7 | 1887 |
| | | | | SEQ ID NO: 2 | | |
| Ab149 | VL CDR | PRT | D1.2 LCDR3 | WO2017009308; | 7 | 1888 |
| | | | | SEQ ID NO: 3 | | |
| Ab150 | VL CDR | PRT | IPN002 LCDR1 | WO2014028777; | 15 | 1889 |
| | | | | SEQ ID NO: 55 | | |
| Ab151 | VL CDR | PRT | IPN002 LCDR2 | WO2014028777; | 32 | 1890 |
| | | | | SEQ ID NO: 56 | | |
| Ab152 | VL CDR | PRT | IPN002 LCDR3 | WO2014028777; | 10 | 1891 |
| | | | | SEQ ID NO: 57 | | |
| Ab153 | VL CDR | PRT | LCDR1 | U.S. Pat. No. 8,980,270; | 16 | 1892 |
| | | | | SEQ ID NO: 1 | | |
| Ab154 | VL CDR | PRT | LCDR1 | U.S. Pat. No. 8,980,270; | 16 | 1893 |
| | | | | SEQ ID NO: 7 | | |
| Ab155 | VL CDR | PRT | LCDR1 | WO2015081085; | 15 | 1894 |
| | | | | SEQ ID NO: 46 | | |
| Ab156 | VL CDR | PRT | LCDR2 | U.S. Pat. No. 8,980,270; | 7 | 1895 |
| | | | | SEQ ID NO: 2 | | |
| Ab157 | VL CDR | PRT | LCDR2 | WO2015081085; | 32 | 1896 |
| | | | | SEQ ID NO: 47 | | |
| Ab158 | VL CDR | PRT | LCDR3 | U.S. Pat. No. 8,980,270; | 9 | 1897 |
| | | | | SEQ ID NO: 3 | | |
| | | | | and 9 | | |
| Ab159 | VL CDR | PRT | MC1 LCDR1 | WO2016137811; | 16 | 1898 |
| | | | | SEQ ID NO: 3 | | |
| Ab160 | VL CDR | PRT | MC1 LCDR2 | WO2016137811; | 8 | 1899 |
| | | | | SEQ ID NO: 4 | | |
| Ab161 | VL CDR | PRT | MC1 LCDR3 | WO2016137811; | 9 | 1900 |
| | | | | SEQ ID NO: 5 | | |
| Ab162 | VL CDR | PRT | PT1 LCDR1 | U.S. Pat. No. 9,371,376; | 12 | 1901 |
| | | | (Chothia) | SEQ ID NO: 22 | | |
| Ab163 | VL CDR | PRT | PT1 LCDR1 | U.S. Pat. No. 9,371,376; | 16 | 1902 |
| | | | (Kabat) | SEQ ID NO: 10 | | |
| Ab164 | VL CDR | PRT | PT1 LCDR2 | U.S. Pat. No. 9,371,376; | 3 | 1903 |
| | | | (Chothia) | SEQ ID NO: 23 | | |
| Ab165 | VL CDR | PRT | PT1 LCDR2 | U.S. Pat. No. 9,371,376; | 7 | 1904 |
| | | | (Kabat) | SEQ ID NO: 11 | | |
| Ab166 | VL CDR | PRT | PT1 LCDR3 | U.S. Pat. No. 9,371,376; | 7 | 1905 |
| | | | (Chothia) | SEQ ID NO: 24 | | |
| Ab167 | VL CDR | PRT | PT1 LCDR3 | U.S. Pat. No. 9,371,376; | 9 | 1906 |
| | | | (Kabat) | SEQ ID NO: 12 | | |
| Ab168 | VL CDR | PRT | PT3 LCDR1 | U.S. Pat. No. 9,371,376; | 7 | 1907 |
| | | | (Chothia) | SEQ ID NO: 28 | | |
| Ab169 | VL CDR | PRT | PT3 LCDR1 | U.S. Pat. No. 9,371,376; | 11 | 1908 |
| | | | (Kabat) | SEQ ID NO: 16 | | |
| Ab170 | VL CDR | PRT | PT3 LCDR2 | U.S. Pat. No. 9,371,376; | 3 | 1909 |
| | | | (Chothia) | SEQ ID NO: 29 | | |
| Ab171 | VL CDR | PRT | PT3 LCDR2 | U.S. Pat. No. 9,371,376; | 7 | 1910 |
| | | | (Kabat) | SEQ ID NO: 17 | | |
| Ab172 | VL CDR | PRT | PT3 LCDR3 | U.S. Pat. No. 9,371,376; | 6 | 1911 |
| | | | (Chothia) | SEQ ID NO: 30 | | |
| Ab173 | VL CDR | PRT | PT3 LCDR3 | U.S. Pat. No. 9,371,376; | 10 | 1912 |
| | | | (Kabat) | SEQ ID NO: 18 | | |
| Ab174 | VL | PRT | 7295-M6 VL | WO2015122922; | 122 | 1913 |
| | | | | SEQ ID NO: 15 | | |
| | | | | and 23 | | |
| Ab175 | VL | PRT | 7297-2M1 VL | WO2015122922; | 116 | 1914 |
| | | | | SEQ ID NO: 71 | | |
| Ab176 | VL | PRT | 7298-M1 VL | WO2015122922; | 121 | 1915 |
| | | | | SEQ ID NO: 31 | | |
| | | | | and 39 | | |
| Ab177 | VL | PRT | 7299-M2 VL | WO2015122922; | 122 | 1916 |
| | | | | SEQ ID NO: 47 | | |
| Ab178 | VL | PRT | 7299-M5 VL | WO2015122922; | 122 | 1917 |
| | | | | SEQ ID NO: 55 | | |
| Ab179 | VL | PRT | 7299-M9 VL | WO2015122922; | 122 | 1918 |
| | | | | SEQ ID NO: 63 | | |
| Ab180 | VL | PRT | C10.2 VL − 107 | | 107 | 1919 |
| Ab181 | VL | PRT | C10.2 VL − 214 | WO2017009308; | 214 | 1920 |
| | | | | SEQ ID NO: 15 | | |
| Ab182 | VL | DNA | C10.2 VL | | 321 | 1921 |
| | | | Bioinformatics | | | |
| Ab183 | VL | DNA | C10.2 VL | | 32 | 1922 |
| | | | EMBOSS | | | |
| Ab184 | VL | DNA | C10.2 VL | | 321 | 1923 |
| | | | GeneInfinity | | | |
| Ab185 | VL | DNA | C10.2 VL | | 321 | 1924 |
| | | | GregThatcher | | | |
| Ab186 | VL | DNA | C10.2 VL IDT | | 321 | 1925 |
| Ab187 | VL | DNA | C10.2 VL | | 321 | 1926 |
| | | | InSilico | | | |
| Ab188 | VL | DNA | C10.2 VL MolBio | | 321 | 1927 |
| Ab189 | VL | DNA | C10.2 VL N2P | | 321 | 1928 |
| Ab190 | VL | DNA | C10.2 VL | | 321 | 1929 |
| | | | Snapgene | | | |
| Ab191 | VL | DNA | C10.2 VL Vector | | 321 | 1930 |
| | | | NTI | | | |
| Ab192 | VL | PRT | C5.2 VL | WO2017009308; | 214 | 1931 |
| | | | | SEQ ID NO: 23 | | |
| Ab193 | VL | PRT | C8.3 VL | WO2017009308; | 214 | 1932 |
| | | | | SEQ ID NO: 31 | | |
| Ab194 | VL | PRT | D1.2 VL | WO2017009308; | 219 | 1933 |
| | | | | SEQ ID NO: 7 | | |
| Ab195 | VL | PRT | D1.2* VL | WO2017009308; | 219 | 1934 |
| | | | | SEQ ID NO: 34 | | |
| Ab196 | VL | PRT | hC10.2 VL | WO2017009308; | 214 | 1935 |
| | | | | SEQ ID NO: 36 | | |
| Ab197 | VL | DNA | IPN001 VL − 336 | U.S. Pat. No. 8,980,270; | 336 | 1936 |
| | | | | SEQ ID NO: 17 | | |
| Ab198 | VL | PRT | IPN001 VL | U.S. Pat. No. 8,980,270; | 112 | 1937 |
| | | | | SEQ ID NO: 13 | | |
| Ab199 | VL | PRT | IPN002 VL | WO2014028777; | 23 | 1938 |
| | | | | SEQ ID NO: 54 | | |
| Ab200 | VL | DNA | IPN002 VL − 336 | U.S. Pat. No. 8,980,270; | 336 | 1939 |
| | | | | SEQ ID NO: 19 | | |
| Ab201 | VL | DNA | IPN002 VL − 714 | | 714 | 1940 |
| Ab202 | VL | PRT | IPN002 VL − 112 | U.S. Pat. No. 8,980,270; | 112 | 1941 |
| | | | | SEQ ID NO: 15 | | |
| Ab203 | VL | DNA | IPN002 VL | U.S. Pat. No. 8,980,270; | 336 | 1942 |
| | | | variant 1 | SEQ ID NO: 32 | | |
| Ab204 | VL | PRT | IPN002 VL | U.S. Pat. No. 8,980,270; | 112 | 1943 |
| | | | variant 1 | SEQ ID NO: 40 | | |
| Ab205 | VL | DNA | IPN002 VL | U.S. Pat. No. 8,980,270; | 336 | 1944 |
| | | | variant 2 | SEQ ID NO: 33 | | |
| Ab206 | VL | PRT | IPN002 VL | U.S. Pat. No. 8,980,270; | 112 | 1945 |
| | | | variant 2 | SEQ ID NO: 41 | | |
| Ab207 | VL | DNA | IPN002 VL | U.S. Pat. No. 8,980,270; | 336 | 1946 |
| | | | variant 3 | SEQ ID NO: 34 | | |
| Ab208 | VL | PRT | IPN002 VL | U.S. Pat. No. 8,980,270; | 112 | 1947 |
| | | | variant 3 | SEQ ID NO: 42 | | |
| Ab209 | VL | DNA | IPN002 VL | U.S. Pat. No. 8,980,270; | 336 | 1948 |
| | | | variant 4 | SEQ ID NO: 35 | | |
| Ab210 | VL | PRT | IPN002 VL | U.S. Pat. No. 8,980,270; | 112 | 1949 |
| | | | variant 4 | SEQ ID NO: 43 | | |
| Ab211 | VL | DNA | MC1 VL − 714 | | 714 | 1950 |
| Ab212 | VL | DNA | MC1 VL − 717 | | 717 | 1951 |
| Ab213 | VL | DNA | MC1 VL − 657 | WO2016137811; | 657 | 1952 |
| | | | | SEQ ID NO: 11 | | |
| Ab214 | VL | PRT | MC1 VL − 238 | | 238 | 1953 |
| Ab215 | VL | PRT | MC1 VL − 219 | WO2016137811; | 219 | 1954 |
| | | | | SEQ ID NO: 1 | | |
| Ab216 | VL | PRT | MC1 VL − 112 | WO2016137811; | 112 | 1955 |
| | | | | SEQ ID NO: 9 | | |
| Ab217 | VL | DNA | PHF1 VL − 339v1 | | 339 | 1956 |
| Ab218 | VL | DNA | PHF1 VL − 339v2 | | 339 | 1957 |
| Ab219 | VL | DNA | PHF1 VL − 396 | | 396 | 1958 |
| Ab220 | VL | DNA | PHF1 VL − 657 | | 657 | 1959 |
| Ab221 | VL | DNA | PHF1 VL − 660 | | 660 | 1960 |
| Ab222 | VL | DNA | PHF1 VL − 678 | | 678 | 1961 |
| Ab223 | VL | DNA | PHF1 VL − 714 | | 714 | 1962 |
| Ab224 | VL | DNA | PHF1 VL − 720 | | 720 | 1963 |
| Ab225 | VL | PRT | PHF1 VL − 239 | | 239 | 1964 |
| Ab226 | VL | DNA | PHF1 VL + (732) | | 732 | 1965 |
| Ab227 | VL | DNA | PHF1 VL + (747) | | 747 | 1966 |
| Ab228 | VL | DNA | PT1 VL − 336 | U.S. Pat. No. 9,371,376; | 336 | 1967 |
| | | | | SEQ ID NO: 32 | | |
| Ab229 | VL | PRT | PT1 VL − 112 | U.S. Pat. No. 9,371,376; | 112 | 1968 |
| | | | | SEQ ID NO: 36 | | |
| Ab230 | VL | DNA | PT3 VL − 321 | U.S. Pat. No. 9,371,376; | 321 | 1969 |
| | | | | SEQ ID NO: 34 | | |
| Ab231 | VL | PRT | PT3 VL − 107 | U.S. Pat. No. 9,371,376; | 107 | 1970 |
| | | | | SEQ ID NO: 38 | | |
| Ab232 | VL | DNA | PT3 VL | | 321 | 1971 |
| | | | Bioinformatics | | | |
| Ab233 | VL | DNA | PT3 VL | | 321 | 1972 |
| | | | Bioinformatics 2 | | | |
| Ab234 | VL | DNA | PT3 VL EMBOSS | | 321 | 1973 |
| Ab235 | VL | DNA | PT3 VL | | 321 | 1974 |
| | | | Geneinfinity | | | |
| Ab236 | VL | DNA | PT3 VL | | 321 | 1975 |
| | | | Genescript | | | |
| Ab237 | VL | DNA | PT3 VL IDT | | 321 | 1976 |
| Ab238 | VL | DNA | PT3 VL NUS | | 321 | 1977 |
| Ab239 | VL | DNA | PT3 VL NUS2 | | 321 | 1978 |
| Ab240 | VL | DNA | PT3 VL NUS3 | | 321 | 1979 |
| Ab241 | VL | DNA | PT3 VL | | 321 | 1980 |
| | | | Snapgene | | | |
| Ab242 | VL | PRT | VL | WO2015081085; | 23 | 1981 |
| | | | | SEQ ID NO: 45 | | |
| Ab243 | Fab | PRT | AT8 Fab1 | Malia, T.J. et al, | 18 | 1982 |
| | | | | “Epitope mapping | | |
| | | | | and structural | | |
| | | | | basis for the | | |
| | | | | recognition | | |
| | | | | of phosphorylated | | |
| | | | | tau by | | |
| | | | | the anti-tau | | |
| | | | | antibody | | |
| | | | | AT8”, | | |
| | | | | Proteins 84 (4), | | |
| | | | | 427-434 (2016), | | |
| | | | | Accession number | | |
| | | | | 5E2V_P | | |
| Ab244 | Fab | PRT | AT8 Fab2 | Malia,T.J. et al, | 18 | 1983 |
| | | | | “Epitope | | |
| | | | | mapping | | |
| | | | | and | | |
| | | | | structural | | |
| | | | | basis for the | | |
| | | | | recognition | | |
| | | | | of phosphorylated | | |
| | | | | tau by | | |
| | | | | the anti-tau | | |
| | | | | antibody | | |
| | | | | AT8”, | | |
| | | | | Proteins 84 (4), | | |
| | | | | 427-434 (2016), | | |
| | | | | Accession | | |
| | | | | number | | |
| | | | | 5E2W_P | | |
| Ab245 | Full | DNA | MC1 Furin 2A- | WO2015035190; | 2168 | 1984 |
| | antibody | | 2168 | SEQ ID NO: 2 | | |
| Ab246 | Full | PRT | MC1 Furin 2A- | WO2015035190; | 718 | 1985 |
| | antibody | | 718 | SEQ ID NO: 4 | | |
| Ab247 | Full | DNA | MC1 optimized | WO2015035190; | 2168 | 1986 |
| | antibody | | seq | SEQ ID NO: 6 | | |
| Ab248 | Full | DNA | PHF1 Furin 2A- | WO2015035190; | 2174 | 1987 |
| | antibody | | 2174 | SEQ ID NO: 1 | | |
| Ab249 | Full | PRT | PHF1 Furin 2A- | WO2015035190; | 720 | 1988 |
| | antibody | | 720 | SEQ ID NO: 3 | | |
| Ab250 | Full | DNA | PHF1 optimized | WO2015035190; | 2174 | 1989 |
| | antibody | | seq | SEQ ID NO: 5 | | |
| Ab251 | VH | DNA | PT3 | | 348 | 2241 |
| Ab252 | VH | DNA | VH (constant- | | 309 | 2242 |
| | Constant | | 309) | | | |
| Ab253 | VH | DNA | VH (constant- | | 291 | 2243 |
| | Constant | | 291) | | | |
| Ab254 | VH | DNA | PHF1 VH − 435 | | 435 | 2169 |
| Ab255 | VH | DNA | PHF1 VH − 408 | | 408 | 2170 |

TABLE 8
Sequences of tau associated disease antibodies

| Ab | | | SEQ |
|---|---|---|---|
| ID | Description | Sequence | ID |
| Ab1 | IPN002 | atgaacttcgggctcagcttgattttccttgtccttgttttaaaagg | 1740 |
| | (VH | tgtccagtgt | |
| | Signal) | | |
| Ab2 | PHF1 (VH | atgggatggagctggatctttctctttctcctgtcaggaactacagg | 1741 |
| | Signal) | tgtcctctct | |
| Ab3 | VH | gccaaaacgacacccccatctgtctatccactggcccctggatctgc | 1742 |
| | (constant- | tgcccaaactaactccatggtgaccctgggatgcctggtcaagggct | |
| | 969) | atttccctgagccagtgacagtgacctggaactctggatccctgtcc | |
| | | agcggtgtgcacaccttcccagctgtcctgcagtctgacctctacac | |
| | | tctgagcagctcagtgactgtcccctccagcacctggcccagcgaga | |
| | | ccgtcacctgcaacgttgcccacccggccagcagcaccaaggtggac | |
| | | aagaaaattgtgcccagggattgtggttgtaagccttgcatatgtac | |
| | | agtcccagaagtatcatctgtcttcatcttccccccaaagcccaagg | |
| | | atgtgctcaccattactctgactcctaaggtcacgtgtgttgtggta | |
| | | gacatcagcaaggatgatcccgaggtccagttcagctggtttgtaga | |
| | | tgatgtggaggtgcacacagctcagacgcaaccccgggaggagcagt | |
| | | tcaacagcactttccgctcagtcagtgaacttcccatcatgcaccag | |
| | | gactggctcaatggcaaggagttcaaatgcagggtcaacagtgcagc | |
| | | tttccctgcccccatcgagaaaaccatctccaaaaccaaaggcagac | |
| | | cgaaggctccacaggtgtacaccattccacctcccaaggagcagatg | |
| | | gccaaggataaagtcagtctgacctgcatgataacagacttcttccc | |
| | | tgaagacattactgtggagtggcagtggaatgggcagccagcggaga | |
| | | actacaagaacactcagcccatcatggacacagatggctcttacttc | |
| | | gtctacagcaagctcaatgtgcagaagagcaactgggaggcaggaaa | |
| | | tactttcacctgctctgtgttacatgagggcctgcacaaccaccata | |
| | | ctgagaagagcctctcccactctcctggt | |
| Ab4 | VH | gccaaaacgacacccccatctgtctatccactggcccctggatctgc | 1743 |
| | (constant- | tgcccaaactaactccatggtgaccctgggatgcctggtcaagggct | |
| | 972) | atttccctgagccagtgacagtgacctggaactctggatccctgtcc | |
| | | agcggtgtgcacaccttcccagctgtcctgcagtctgacctctacac | |
| | | tctgagcagctcagtgactgtcccctccagcacctggcccagcgaga | |
| | | ccgtcacctgcaacgttgcccacccggccagcagcaccaaggtggac | |
| | | aagaaaattgtgcccagggattgtggttgtaagccttgcatatgtac | |
| | | agtcccagaagtatcatctgtcttcatcttccccccaaagcccaagg | |
| | | atgtgctcaccattactctgactcctaaggtcacgtgtgttgtggta | |
| | | gacatcagcaaggatgatcccgaggtccagttcagctggtttgtaga | |
| | | tgatgtggaggtgcacacagctcagacgcaaccccgggaggagcagt | |
| | | tcaacagcactttccgctcagtcagtgaacttcccatcatgcaccag | |
| | | gactggctcaatggcaaggagttcaaatgcagggtcaacagtgcagc | |
| | | tttccctgcccccatcgagaaaaccatctccaaaaccaaaggcagac | |
| | | cgaaggctccacaggtgtacaccattccacctcccaaggagcagatg | |
| | | gccaaggataaagtcagtctgacctgcatgataacagacttcttccc | |
| | | tgaagacattactgtggagtggcagtggaatgggcagccagcggaga | |
| | | actacaagaacactcagcccatcatggacacagatggctcttacttc | |
| | | gtctacagcaagctcaatgtgcagaagagcaactgggaggcaggaaa | |
| | | tactttcacctgctctgtgttacatgagggcctgcacaaccaccata | |
| | | ctgagaagagcctctcccactctcctggtaag | |
| Ab5 | 7295-M6 | GFNIKDYYIH | 1744 |
| | HCDR | | |
| Ab6 | 7295-M6 | WIDPENGDTEYAPKFQ | 1745 |
| | HCDR | | |
| Ab7 | 7295-M6 | APGY | 1746 |
| | HCDR | | |
| Ab8 | 7297-2M1 | NIDPSDSETHYNQKFK | 1747 |
| | HCDR | | |
| Ab9 | 7298-M1 | SGFTFTDYYMS | 1748 |
| | HCDR | | |
| Ab10 | 7298-M1 | NRNKTKGYTTEYSASV | 1749 |
| | HCDR | | |
| Ab11 | 7298-M1 | GMDY | 1750 |
| | HCDR | | |
| Ab12 | 7298-M2 | GFTFSTYPMS | 1751 |
| | HCDR | | |
| Ab13 | 7298-M2 | SISNGGSTYYPDTV | 1752 |
| | HCDR | | |
| Ab14 | 7298-M2 | GRDYHFDF | 1753 |
| | HCDR | | |
| Ab15 | 7299-M2 | GYTFTNYWIN | 1754 |
| | HCDR | | |
| Ab16 | 7299-M2 | IYPGSTRANYNEKFKS | 1755 |
| | HCDR | | |
| Ab17 | 7299-M2 | THSI | 1756 |
| | HCDR | | |
| Ab18 | 7299-M5 | GYTFISNWMH | 1757 |
| | HCDR | | |
| Ab19 | 7299-M5 | IDPSDSETHYNQKFKD | 1758 |
| | HCDR | | |
| Ab20 | 7299-M5 | RDRDGYYFDY | 1759 |
| | HCDR | | |
| Ab21 | 7299-M9 | IYPGSTRANYNEKFK | 1760 |
| | HCDR | | |
| Ab22 | C10.2 | KASGYTFTDRTIH | 1761 |
| | HCDR1 | | |
| Ab23 | C10.2 | YIYPGDGSTKYNENFK | 1762 |
| | HCDR2 | | |
| Ab24 | C10.2 | RGAMDY | 1763 |
| | HCDR3 | | |
| Ab25 | C5.2 | YIYPGDDSTKYNDNFK | 1764 |
| | HCDR2 | | |
| Ab26 | C5.2 | RGTMDY | 1765 |
| | HCDR3 | | |
| Ab27 | D1.2 | KASGNTFTDYEIH | 1766 |
| | HCDR1 | | |
| Ab28 | D1.2 | AIDPETGNTAYNQKFK | 1767 |
| | HCDR2 | | |
| Ab29 | D1.2 | SRGFDY | 1768 |
| | HCDR3 | | |
| Ab30 | HCDR1 | SYGMS | 1769 |
| Ab31 | HCDR1 | KYGMS | 1770 |
| Ab32 | HCDR2 | TISSSGSRTYFPDSVKG | 1771 |
| Ab33 | HCDR2 | TISSSGSRTYYPDSVKG | 1772 |
| Ab34 | HCDR2 | RFTISRDNAKNTLYLQMSSSKSEDTAMYYCSI | 1773 |
| Ab35 | HCDR3 | TWDGAMDY | 1774 |
| Ab36 | HCDR3 | SWDGAMDY | 1775 |
| Ab37 | HCDR3 | WGQGTSVTVSS | 1776 |
| Ab38 | IPN002 | WVRQAPGKGLEWVA | 1777 |
| | HCDR1 | | |
| Ab39 | IPN002 | RFTISRDNAKNTLYLQMXSXXXEDTAMYYCXI | 1778 |
| | HCDR2 | | |
| Ab40 | IPN002 | WGQGTXVTVSS | 1779 |
| | HCDR3 | | |
| Ab41 | MC1 | KGSGYTFSNYWIE | 1780 |
| | LCDR1 | | |
| Ab42 | MC1 | EILPGSDSIKYEKNFKG | 1781 |
| | LCDR2 | | |
| Ab43 | MC1 | ARRGNYVDD | 1782 |
| | LCDR3 | | |
| Ab44 | PT1 | GYTFSSS | 1783 |
| | HCDR1 | | |
| | (Chothia) | | |
| Ab45 | PT1 | SSWMG | 1784 |
| | HCDR1 | | |
| | (Kabat) | | |
| Ab46 | PT1 | LPGSGG | 1785 |
| | HCDR2 | | |
| | (Chothia) | | |
| Ab47 | PT1 | DILPGSGGTNYNERFKG | 1786 |
| | HCDR2 | | |
| | (Kabat) | | |
| Ab48 | PT1 | SYYDYDRFA | 1787 |
| | HCDR3 | | |
| | (Chothia) | | |
| Ab49 | PT1 | SYYDYDRFAN | 1788 |
| | HCDR3 | | |
| | (Kabat) | | |
| Ab50 | PT3 | GFTFSSY | 1789 |
| | HCDR1 | | |
| | (Chothia) | | |
| Ab51 | PT3 | SYAMS | 1790 |
| | HCDR1 | | |
| | (Kabat) | | |
| Ab52 | PT3 | SKGGN | 1791 |
| | HCDR2 | | |
| | (Chothia) | | |
| Ab53 | PT3 | SISKGGNTYYPNSVKG | 1792 |
| | HCDR2 | | |
| | (Kabat) | | |
| Ab54 | PT3 | GWGDYGWFA | 1793 |
| | HCDR3 | | |
| | (Chothia) | | |
| Ab55 | PT3 | GWGDYGWFAY | 1794 |
| | HCDR3 | | |
| | (Kabat) | | |
| Ab56 | 7295-M6 | LPEVQLEESGAELVRSGASVTLSCTASGFNIKDYYIHWVKQRPEQGL | 1795 |
| | VH | EWIGWIDPENGDTEYAPKFQGKATMTADTSSNTAYLQLSSLTSEDTA | |
| | | VYYCNGAPGYWGPGTTLTVSSAKTIPPSVYS | |
| Ab57 | 7297-2M1 | LPEVQLEQSGAELVKPGASVKLSCKASGYTFISNWMHWVKQRPGQGL | 1796 |
| | VH | EWIGNIDPSDSETHYNQKFKDKATLTVDKSSSTAYMQLSSLTSEDSA | |
| | | VYYCARRDRDGYYFDYWGQGTTLTVSSAKTTPPSVYS | |
| Ab58 | 7298-M1 | LPEVQLEESGGGLVQPGGSLSLSCAASGFTFTDYYMSWVRQPPGKAL | 1797 |
| | VH | EWVALNRNKTKGYTTEYSASVKGRFTISRDNSQSILYLQMNALRAED | |
| | | SATYYCARGMDYWGQGTSVTVSSAKTTPPSVYS | |
| Ab59 | 7298-M2 | LPEVQLEESGGGLVKPGGSLKLSCAASGFTFSTYPMSWVRQTPEKRL | 1798 |
| | VH | EWVASISNGGSTYYPDTVKGRFTISRDNARNILYLQMSSLRSEDTAM | |
| | | YYCARGRDYHFDFWGQGTTLTVSSAKTIPPSVYS | |
| Ab60 | 7299-M2 | LPQVQLEESGAELVKPGASVKLSCKASGYTFTNYWINWVKQRPGQGL | 1799 |
| | VH | EWIGNIYPGSTRANYNEKFKSKATLTVDISSSTAYMQVSSLISDDSA | |
| | | VYYCTRTHSIWGQGTQVTVSAAKTTPPSVYS | |
| Ab61 | 7299-M5 | LPEVKLEESGAELVKPGASVKLSCKASGYTFISNWMHWVKQRPGQGL | 1800 |
| | VH | EWIGNIDPSDSETHYNQKFKDKATLTVDKSSSTAYMQLSSLISEDSA | |
| | | VYYCARRDRDGYYFDYWGQGTTLTVSSAKTTPPSVYS | |
| Ab62 | 7299-M9 | LPEVKLEQSGAELVKPGASVKLSCKASGYTFTNYWINWVKQRPGQGL | 1801 |
| | VH | EWIGNIYPGSTRANYNEKFKSKATLTVDISSSTAYMQVSSLTSDDSA | |
| | | VYYCTRTHSIWGQGTQVTVSAAKTIPPSVYS | |
| Ab63 | C10.2 VH | QVQLQQSDAELVKPGASVKISCKASGYTFTDRTIHWVKQRPEQGLEW | 1802 |
| | | IGYIYPGDGSTKYNENFKGKATLIADKSSSTAYMQLNSLTSEDSAVY | |
| | | FCARRGAMDYWGQGTSVTVSSAKTTPPSVYPLAPGSAAQTNSMVTLG | |
| | | CLVKGYFPEPVTVTWNSGSLSSGVHTFPAVLQSDLYTLSSSVTVPSS | |
| | | TWPSETVTCNVAHPASSTKVDKKIVPRDCGCKPCICTVPEVSSVFIF | |
| | | PPKPKDVLTITLTPKVTCVVVDISKDDPEVQFSWFVDDVEVHTAQTQ | |
| | | PREEQFNSIFRSVSELPIMHQDWLNGKEFKCRVNSAAFPAPIEKTIS | |
| | | KTKGRPKAPQVYTIPPPKEQMAKDKVSLTCMITDFFPEDITVEWQWN | |
| | | GQPAENYKNTQPIMDTDGSYFVYSKLNVQKSNWEAGNTFTCSVLHEG | |
| | | LHNHHTEKSLSHSPGK | |
| Ab64 | C10.2 VH- | QVQLQQSDAELVKPGASVKISCKASGYTFTDRTIHWVKQRPEQGLEW | 1803 |
| | 115 | IGYIYPGDGSTKYNENFKGKATLTADKSSSTAYMQLNSLTSEDSAVY | |
| | | FCARRGAMDYWGQGTSVTVSS | |
| Ab65 | C10.2 VH | caggtgcagctgcagcagagcgacgccgagctggtgaagcccggcgc | 1804 |
| | Bioinformatics | cagcgtgaagatcagctgcaaggccagcggctacaccttcaccgaca | |
| | | ggaccatccactgggtgaagcagaggcccgagcagggcctggagtgg | |
| | | atcggctacatctaccccggcgacggcagcaccaagtacaacgagaa | |
| | | cttcaagggcaaggccaccctgaccgccgacaagagcagcagcaccg | |
| | | cctacatgcagctgaacagcctgaccagcgaggacagcgccgtgtac | |
| | | ttctgcgccaggaggggcgcgatggactactggggccagggcaccag | |
| | | cgtgaccgtgagcagc | |
| Ab66 | C10.2 VH | caggtgcagctgcagcagagcgacgccgagctggtgaagcccggcgc | 1805 |
| | EMBOSS | cagcgtgaagatcagctgcaaggccagcggctacaccttcaccgacc | |
| | | ggaccatccactgggtgaagcagcggcccgagcagggcctggagtgg | |
| | | atcggctacatctaccccggcgacggcagcaccaagtacaacgagaa | |
| | | cttcaagggcaaggccaccctgaccgccgacaagagcagcagcaccg | |
| | | cctacatgcagctgaacagcctgaccagcgaggacagcgccgtgtac | |
| | | ttctgcgcccggcggggcgctatggactactggggccagggcaccag | |
| | | cgtgaccgtgagcagc | |
| Ab67 | C10.2 VH | caggtgcagctgcagcagagcgacgccgagctggtgaagcccggcgc | 1806 |
| | GeneInfinity | cagcgtgaagatcagctgcaaggccagcggctacaccttcaccgacc | |
| | | gaaccatccactgggtgaagcagcgacccgagcagggcctggagtgg | |
| | | atcggctacatctaccccggcgacggcagcaccaagtacaacgagaa | |
| | | cttcaagggcaaggccaccctgaccgccgacaagagcagcagcaccg | |
| | | cctacatgcagctgaacagcctgaccagcgaggacagcgccgtgtac | |
| | | ttctgcgcccgacgaggcgctatggactactggggccagggcaccag | |
| | | cgtgaccgtgagcagc | |
| Ab68 | C10.2 VH | caggtgcagctgcagcagagcgatgccgagctggtgaaacctggggc | 1807 |
| | GregThatcher | cagcgtcaagatcagctgcaaggcctccgggtacaccttcaccgacc | |
| | | gcaccatccactgggtgaagcagcggcccgagcaggggctggagtgg | |
| | | atcggctacatctaccccggggacggcagcaccaagtacaacgagaa | |
| | | cttcaagggcaaggccaccctgaccgccgacaagtccagcagcacgg | |
| | | cctacatgcagctcaacagcctcaccagcgaggacagcgccgtgtac | |
| | | ttctgcgcccggcggggcgcaatggactactggggccagggcacctc | |
| | | cgtgaccgtgtcctcc | |
| Ab69 | C10.2 VH | caggtgcagctgcagcagagcgatgccgagctggtgaaacccggggc | 1808 |
| | IDT | ctccgtcaagatcagctgcaaggccagcgggtacaccttcaccgacc | |
| | | gcaccatccactgggtgaagcagcggcccgagcagggcctggagtgg | |
| | | atcggctacatctaccccggggacggcagcaccaagtacaacgagaa | |
| | | cttcaagggcaaggccaccctgaccgccgacaagtccagcagcacag | |
| | | cctacatgcagctcaacagcctcacctctgaggacagcgccgtgtac | |
| | | ttctgcgcccggcggggcgcgatggactactggggccaggggaccag | |
| | | cgtgaccgtgtcctcc | |
| Ab70 | C10.2 VH | caggtgcagctgcagcagagcgacgccgagctggtgaaacctggggc | 1809 |
| | InSilico | cagcgtcaagatcagctgcaaggcctccggctacaccttcaccgacc | |
| | | gcaccatccactgggtgaagcagcggcccgagcaggggctggagtgg | |
| | | atcgggtacatctaccccggggacggctccaccaagtacaacgagaa | |
| | | cttcaagggcaaagccaccctgacggccgacaagtccagcagcaccg | |
| | | cctacatgcagctgaacagcctgaccagcgaggacagcgccgtgtac | |
| | | ttctgcgcccggggggggctatggactactggggccagggcacctc | |
| | | cgtgaccgtgtcctcc | |
| Ab71 | C10.2 VH | caggtgcagctgcagcagtccgacgccgagctggtgaagcccggcgc | 1810 |
| | MolBio | ctccgtgaagatctcctgcaaggcctccggctacaccttcaccgacc | |
| | | ggaccatccactgggtgaagcagcggcccgagcagggcctggagtgg | |
| | | atcggctacatctaccccggcgacggctccaccaagtacaacgagaa | |
| | | cttcaagggcaaggccaccctgaccgccgacaagtcctcctccaccg | |
| | | cctacatgcagctgaactccctgacctccgaggactccgccgtgtac | |
| | | ttctgcgcccggcggggcgctatggactactggggccagggcacctc | |
| | | cgtgaccgtgtcctcc | |
| Ab72 | C10.2 VH | caggtgcagctgcagcagtccgatgccgagctggtgaaaccgggggc | 1811 |
| | N2P | cagcgtcaagatctcatgcaaagcttccggctacaccttcaccgaca | |
| | | gaaccatccactgggtgaagcagcggcccgagcaggggctggagtgg | |
| | | atcgggtacatctaccccggggacggcagcaccaagtacaacgagaa | |
| | | cttcaagggcaaggccaccctgacagccgacaagtccagctccaccg | |
| | | cctacatgcagctcaacagcctgacctccgaggacagcgccgtgtac | |
| | | ttctgcgcccggcggggcgcaatggactactggggccagggcaccag | |
| | | cgtgaccgtgagcagc | |
| Ab73 | C10.2 VH | caggtgcagctgcagcagagcgacgccgagctggtgaagcctggcgc | 1812 |
| | Snapgene | cagcgtgaagatcagctgcaaggccagcggctacaccttcaccgacc | |
| | | gtaccatccactgggtgaagcagcgacctgagcagggcctggagtgg | |
| | | atcggctacatctaccctggcgacggcagcaccaagtacaacgagaa | |
| | | cttcaagggcaaggccaccctgaccgccgacaagagcagcagcaccg | |
| | | cctacatgcagctgaacagcctgaccagcgaggacagcgccgtgtac | |
| | | ttctgcgcccgtcgaggcgctatggactactggggccagggcaccag | |
| | | cgtgaccgtgagcagc | |
| Ab74 | C10.2 VH | caggtgcagctgcagcagagcgacgccgagctggtgaagcctggcgc | 1813 |
| | Vector NTI | cagcgtgaagatcagctgcaaggccagcggctacaccttcaccgacc | |
| | | gtaccatccactgggtgaagcagcgacctgagcagggcctggagtgg | |
| | | atcggctacatctaccccggcgacggcagcaccaagtacaacgagaa | |
| | | cttcaagggcaaggccaccctgaccgccgacaagagcagcagcaccg | |
| | | cctacatgcagctgaacagcctgaccagcgaggacagcgccgtgtac | |
| | | ttctgcgcccggcggggcgctatggactactggggccagggcaccag | |
| | | cgtgaccgtgagcagc | |
| Ab75 | C5.2 VH | QVQLQQSDAELVKPGASVKISCKASGYTFTDRTIHWVKQRPEQGLEW | 1814 |
| | | IGYIYPGDDSTKYNDMFKAKATLTADKSSNTAYMQLNSLTSDDSAVY | |
| | | FCARRGTMDYWGQGISVTVSSAKTTPPSVYPLAPGSAAQTNSMVTLG | |
| | | CLVKGYFPEPVTVTWNSGSLSSGVHTFPAVLQSDLYTLSSSVTVPSS | |
| | | TWPSETVTCNVAHPASSTKVDKKIVPRDCGCKPCICTVPEVSSVFIF | |
| | | PPKPKDVLTITLTPKVTCVVVDISKDDPEVQFSWFVDDVEVHTAQTQ | |
| | | PREEQFNSTFRSVSELPIMHQDWLNGKEFKCRVNSAAFPAPIEKTIS | |
| | | KTKGRPKAPQVYTIPPPKEQMAKDKVSLTCMITDFFPEDITVEWQWN | |
| | | GQPAENYKNTQPIMDTDGSYFVYSKLNVQKSNWEAGNTFTCSVLHEG | |
| | | LHNHHTEKSLSHSPGK | |
| Ab76 | C8.3 VH | QVQLQQSDAELVNPGASVKISCKASGYTFTDRTIHWVKQRPEQGLEW | 1815 |
| | | IGYIYPGDGSTKYNENFKGKATLTADKSSSTAYMQLNSLASEDSAVY | |
| | | FCARRGAMDYWGQGTSVTVSSAKTTPPSVYPLAPGSAAQTNSMVTLG | |
| | | CLVKGYFPEPVTVTWNSGSLSSGVHTFPAVLQSDLYTLSSSVTVPSS | |
| | | TWPSETVTCNVAHPASSTKVDKKIVPRDCGCKPCICTVPEVSSVFIF | |
| | | PPKPKDVLIITLTPKVTCVVVDISKDDPEVQFSWFVDDVEVHTAQTQ | |
| | | PREEQFNSTERSVSELPIMHQDWLNGKEFKCRVNSAAFPAPIEKTIS | |
| | | KTKGRPKAPQVYTIPPPKEQMAKDKVSLTCMITDFFPEDITVEWQWN | |
| | | GQPAENYKNTQPIMDTDGSYFVYSKLNVQKSNWEAGNTFTCSVLHEG | |
| | | LHNHHTEKSLSHSPGK | |
| Ab77 | D1.2 VH | QVQLQQSGAELVRPGASVILSCKASGNIFIDYEIHWVKQTPVHGLEW | 1816 |
| | | IGAIDPETGNTAYNQKFKGKARLTADKSSSTAYMELRSLTSEDSAVY | |
| | | YCTRSRGFDYWGQGTTLTVSSAKTTPPSVYPLAPGCGDTTGSSVTLG | |
| | | CLVKGYFPESVTVTWNSGSLSSSVHTFPALLQSGLYTMSSSVTVPSS | |
| | | TWPSQTVTCSVAHPASSTTVDKKLEPSGPISTINPCPPCKECHKCPA | |
| | | PNLEGGPSVFIFPPNIKDVLMISLTPKVTCVVVDVSEDDPDVRISWF | |
| | | VNNVEVHTAQTQTHREDYNSTIRVVSALPIQHQDWMSGKEFKCKVNN | |
| | | KDLPSPIERTISKIKGLVRAPQVYILPPPAEQLSRKDVSLTCLVVGF | |
| | | NPGDISVEWTSNGHTEENYKDTAPVLDSDGSYFIYSKLDIKTSKWEK | |
| | | TDSFSCNVRHEGLKNYYLKKTISRSPGK | |
| Ab78 | hC10.2 VH | QVQLVQSGAEVVKPGASVKISCKASGYTFTDRTIHWVRQAPGQGLEW | 1817 |
| | | IGYIYPGDGSTKYSQKFQGRATLTADTSASTAYMELSSLRSEDTAVY | |
| | | YCARRGAMDYWGQGTSVTVSSASTKGPSVFPLAPSSKSTSGGTAALG | |
| | | CLVKDYFPEPVTVSWNSGALISGVHTFPAVLQSSGLYSLSSVVTVPS | |
| | | SSLGTQTYICNVNHKPSNTKVDKRVEPKSCDKTHTCPPCPAPELLGG | |
| | | PSVFLFPPKPKDTLMISRTPEVTCVVVDVSHEDPEVKFNWYVDGVEV | |
| | | HNAKTKPREEQYNSTYRVVSVLTVLHQDWLNGKEYKCKVSNKALPAP | |
| | | IEKTISKAKGQPREPQVYTLPPSREEMTKNQVSLTCLVKGFYPSDIA | |
| | | VEWESNGQPENNYKTTPPVLDSDGSFFLYSKLTVDKSRWQQGNVFSC | |
| | | SVMHEALHNHYTQKSLSLSPG | |
| Ab79 | IPN001 | gaggtgcagttggtggagtctggggaagacttagtgaagcctggagg | 1818 |
| | VH-351 | gtccctgaaactctcctgtgtcgcttctggattcgctttcagtagct | |
| | | atggcatgtcttgggttcgccagactccagacatgaggctggagtgg | |
| | | gtcgcaacaattagtagcagtggtagtcgcacctactttccagacag | |
| | | tgtgaaggggcgactcaccatctccagagacaatgacaagaacatcc | |
| | | tatacctacaaatgagcagtctgaggtctgaggacacagccatgtac | |
| | | tattgtacgattacctgggacggtgctatggactactggggtcgtgg | |
| | | aatatcagtcaccgtctcctca | |
| Ab80 | IPN001 | EVQLVESGEDLVKPGGSLKLSCVASGFAFSSYGMSWVRQTPDMRLEW | 1819 |
| | VH-117 | VATISSSGSRTYFPDSVKGRLTISRDNDKNILYLQMSSLRSEDTAMY | |
| | | YCTITWDGAMDYWGRGISVTVSS | |
| Ab81 | IPN002 | atgaacttcgggctcagcttgattttccttgtccttgttttaaaagg | 1820 |
| | VH-1377 | tgtccagtgtgaggttcatctggtggagtctgggggagccttagtga | |
| | | agcctggagggtccctgaaactctcctgtgcagcctctggattcagt | |
| | | ttcagtaaatatggcatgtcttgggttcgccagactccagacaagag | |
| | | gctggagtgggtcgcaaccattagtagtagtgggagtcgcacctact | |
| | | atccagacagtgtgaagggccaattcaccatctccagagacaatgcc | |
| | | aagaacaccctgtacctgcaaatgagcagtctgaagtctgaggacac | |
| | | agccatgtattactgttcaattagctgggacggtgctatggactact | |
| | | ggggtcaagggacctcagtcaccgtctcctcagccaaaacgacaccc | |
| | | ccatctgtctatccactggcccctggatctgctgcccaaactaactc | |
| | | catggtgaccctgggatgcctggtcaagggctatttccctgagccag | |
| | | tgacagtgacctggaactctggatccctgtccagcggtgtgcacacc | |
| | | ttcccagctgtcctgcagtctgacctctacactctgagcagctcagt | |
| | | gactgtcccctccagcacctggcccagcgagaccgtcacctgcaacg | |
| | | ttgcccacccggccagcagcaccaaggtggacaagaaaattgtgccc | |
| | | agggattgtggttgtaagccttgcatatgtacagtcccagaagtatc | |
| | | atctgtcttcatcttccccccaaagcccaaggatgtgctcaccatta | |
| | | ctctgactcctaaggtcacgtgtgttgtggtagacatcagcaaggat | |
| | | gatcccgaggtccagttcagctggtttgtagatgatgtggaggtgca | |
| | | cacagctcagacgcaaccccgggaggagcagttcaacagcactttcc | |
| | | gctcagtcagtgaacttcccatcatgcaccaggactggctcaatggc | |
| | | aaggagttcaaatgcagggtcaacagtgcagctttccctgcccccat | |
| | | cgagaaaaccatctccaaaaccaaaggcagaccgaaggctccacagg | |
| | | tgtacaccattccacctcccaaggagcagatggccaaggataaagtc | |
| | | agtctgacctgcatgataacagacttcttccctgaagacattactgt | |
| | | ggagtggcagtggaatgggcagccagcggagaactacaagaacactc | |
| | | agcccatcatggacacagatggctcttacttcgtctacagcaagctc | |
| | | aatgtgcagaagagcaactgggaggcaggaaatactttcacctgctc | |
| | | tgtgttacatgagggcctgcacaaccaccatactgagaagagcctct | |
| | | cccactctcctggt | |
| Ab82 | IPN002 | gaggttcatctggtggagtctgggggagccttagtgaagcctggagg | 1821 |
| | VH-351 | gtccctgaaactctcctgtgcagcctctggattcagtttcagtaaat | |
| | | atggcatgtcttgggttcgccagactccagacaagaggctggagtgg | |
| | | gtcgcaaccattagtagtagtgggagtcgcacctactatccagacag | |
| | | tgtgaagggccaattcaccatctccagagacaatgccaagaacaccc | |
| | | tgtacctgcaaatgagcagtctgaagtctgaggacacagccatgtat | |
| | | tactgttcaattagctgggacggtgctatggactactggggtcaagg | |
| | | gacctcagtcaccgtctcctca | |
| Ab83 | IPN002 | EVHLVESGGALVKPGGSLKLSCAASGFSFSKYGMSWVRQTPDKRLEW | 1822 |
| | VH-117 | VATISSSGSRTYYPDSVKGQFTISRDNAKNTLYLQMSSLKSEDTAMY | |
| | | YCSISWDGAMDYWGQGTSVTVSS | |
| Ab84 | IPN002 | EVXLVESGGALVKPGGSLRLSCAASGFSFS | 1823 |
| | VH-30 | | |
| Ab85 | IPN002 | gaggttcatctggtggagtctgggggagccttagtgaagcctggagg | 1824 |
| | VH variant | gtccctgagactctcctgtgcagcctctggattcagtttcagtaaat | |
| | 1 | atggcatgtcttgggttcgccaggccccaggcaagggcctggagtgg | |
| | | gtcgcaaccattagtagtagtgggagtcgcacctactatccagacag | |
| | | tgtgaagggcagattcaccatctccagagacaatgccaagaacaccc | |
| | | tgtacctgcaaatgagcagtctgaagtctgaggacacagccatgtat | |
| | | tactgttcaattagctgggacggtgctatggactactggggtcaagg | |
| | | gacctcagtcaccgtctcctca | |
| Ab86 | IPN002 | EVHLVESGGALVKPGGSLRLSCAASGFSFSKYGMSWVRQAPGKGLEW | 1825 |
| | VH variant | VATISSSGSRTYYPDSVKGRFTISRDNAKNTLYLQMSSLKSEDTAMY | |
| | 1 | YCSISWDGAMDYWGQGTSVTVSS | |
| Ab87 | IPN002 | gaggttcatctggtggagtctgggggagccttagtgaagcctggagg | 1826 |
| | VH variant | gtccctgagactctcctgtgcagcctctggattcagtttcagtaaat | |
| | 2 | atggcatgtcttgggttcgccaggccccaggcaagggcctggagtgg | |
| | | gtcgcaaccattagtagtagtgggagtcgcacctactatccagacag | |
| | | tgtgaagggcagattcaccatctccagagacaatgccaagaacaccc | |
| | | tgtacctgcaaatgaacagtctgagagccgaggacacagccatgtat | |
| | | tactgttcaattagctgggacggtgctatggactactggggtcaagg | |
| | | gaccaccgtcaccgtctcctca | |
| Ab88 | IPN002 | EVHLVESGGALVKPGGSLRLSCAASGFSFSKYGMSWVRQAPGKGLEW | 1827 |
| | VH variant | VATISSSGSRTYYPDSVKGRFTISRDNAKNTLYLQMNSLRAEDTAMY | |
| | 2 | YCSISWDGAMDYWGQGTTVTVSS | |
| Ab89 | IPN002 | gaggttcagctggtggagtctgggggagccttagtgaagcctggagg | 1828 |
| | VH variant | gtccctgagactctcctgtgcagcctctggattcagtttcagtaaat | |
| | 3 | atggcatgtcttgggttcgccaggccccaggcaagggcctggagtgg | |
| | | gtcgcaaccattagtagtagtgggagtcgcacctactatccagacag | |
| | | tgtgaagggcagattcaccatctccagagacaatgccaagaacaccc | |
| | | tgtacctgcaaatgaacagtctgagagccgaggacacagccatgtat | |
| | | tactgttcaattagctgggacggtgctatggactactggggtcaagg | |
| | | gaccaccgtcaccgtctcctca | |
| Ab90 | IPN002 | EVQLVESGGALVKPGGSLRLSCAASGFSFSKYGMSWVRQAPGKGLEW | 1829 |
| | VH variant | VATISSSGSRTYYPDSVKGRFTISRDNAKNTLYLQMNSLRAEDTAMY | |
| | 3 | YCSISWDGAMDYWGQGTTVTVSS | |
| Ab91 | IPN002 | gaggttcagctggtggagtctgggggagccttagtgaagcctggagg | 1830 |
| | VH variant | gtccctgagactctcctgtgcagcctctggattcagtttcagtaaat | |
| | 4 | atggcatgtcttgggttcgccaggccccaggcaagggcctggagtgg | |
| | | gtcgcaaccattagtagtagtgggagtcgcacctactatccagacag | |
| | | tgtgaagggcagattcaccatctccagagacaatgccaagaacaccc | |
| | | tgtacctgcaaatgaacagtctgagagccgaggacacagccatgtat | |
| | | tactgtgccattagctgggacggtgctatggactactggggtcaagg | |
| | | gaccaccgtcaccgtctcctca | |
| Ab92 | IPN002 | EVQLVESGGALVKPGGSLRLSCAASGFSFSKYGMSWVRQAPGKGLEW | 1831 |
| | VH variant | VATISSSGSRTYYPDSVKGRFTISRDNAKNTLYLQMNSLRAEDTAMY | |
| | 4 | YCAISWDGAMDYWGQGTTVTVSS | |
| Ab93 | MC1 VH- | atggaatggacctgggtctttctcttcctcctgtcagtaactgcagg | 1832 |
| | 1374 | tgtccactcccaggttcagctgcagcagtctggagctgagctgatga | |
| | | agcctggggcctcagtgaagatatcctgcaaggctactggctacaca | |
| | | ttcagtaactactggatagagtgggtaaagcagaggcctggacatgg | |
| | | ccttgagtggattggagagattttacctggaagtgatagtattaagt | |
| | | acaatgagaatttcaagggcaaggccacattcactgcagatacatcc | |
| | | tccaacacagcctacatgcaactcagcagcctgacatctgaggactc | |
| | | tgccgtctattactgtgcaagaagggggaactacccagactactggg | |
| | | gccaaggcaccactctcacagtctcctcagccaaaacgacaccccca | |
| | | tctgtctatccactggcccctggatctgctgcccaaactaactccat | |
| | | ggtgaccctgggatgcctggtcaagggctatttccctgagccagtga | |
| | | cagtgacctggaactctggatccctgtccagcggtgtgcacaccttc | |
| | | ccagctgtcctgcagtctgacctctacactctgagcagctcagtgac | |
| | | tgtcccctccagcacctggcccagcgagaccgtcacctgcaacgttg | |
| | | cccacccggccagcagcaccaaggtggacaagaaaattgtgcccagg | |
| | | gattgtggttgtaagccttgcatatgtacagtcccagaagtatcatc | |
| | | tgtcttcatcttccccccaaagcccaaggatgtgctcaccattactc | |
| | | tgactcctaaggtcacgtgtgttgtggtagacatcagcaaggatgat | |
| | | cccgaggtccagttcagctggtttgtagatgatgtggaggtgcacac | |
| | | agctcagacgcaaccccgggaggagcagttcaacagcactttccgct | |
| | | cagtcagtgaacttcccatcatgcaccaggactggctcaatggcaag | |
| | | gagttcaaatgcagggtcaacagtgcagctttccctgcccccatcga | |
| | | gaaaaccatctccaaaaccaaaggcagaccgaaggctccacaggtgt | |
| | | acaccattccacctcccaaggagcagatggccaaggataaagtcagt | |
| | | ctgacctgcatgataacagacttcttccctgaagacattactgtgga | |
| | | gtggcagtggaatgggcagccagcggagaactacaagaacactcagc | |
| | | ccatcatggacacagatggctcttacttcgtctacagcaagctcaat | |
| | | gtgcagaagagcaactgggaggcaggaaatactttcacctgctctgt | |
| | | gttacatgagggcctgcacaaccaccatactgagaagagcctctccc | |
| | | actctcctggt | |
| Ab94 | MC1 VH- | gaggtgcagctggtgcagtctggagcagaggtgaaaaagcccgggga | 1833 |
| | 1326 | gtctctgaagatctcctgtaagggttctggctacacattcagtaact | |
| | | actggatagagtgggtgcgccagatgcccgggaaaggcctggagtgg | |
| | | atgggggagattttacctggaagtgatagtattaagtacgaaaagaa | |
| | | tttcaagggccaggtcaccatctcagccgacaagtccatcagcaccg | |
| | | cctacctgcagtggagcagcctgaaggcctcggacaccgccatgtat | |
| | | tactgtgcgagaagggggaactacgtggacgactggggccagggcac | |
| | | cctggtcaccgtctcctcagcttctaccaagggcccatcggtcttcc | |
| | | cgctagcgccctgctccaggagcacctccgagagcacagccgccctg | |
| | | ggctgcctggtcaaggactacttccccgaaccggtgacggtgtcgtg | |
| | | gaactcaggcgccctgaccagcggcgtgcacaccttcccggctgtcc | |
| | | tacagtcctcaggactctactccctcagcagcgtggtgaccgtgccc | |
| | | tccagcagcttgggcacgaagacctacacctgcaacgtagatcacaa | |
| | | gcccagcaacaccaaggtggacaagagagttgagtccaaatatggtc | |
| | | ccccatgcccaccctgcccagcacctgaggccgccgggggaccatca | |
| | | gtcttcctgttccccccaaaacccaaggacactctcatgatctcccg | |
| | | gacccctgaggtcacgtgcgtggtggtggacgtgagccaggaagacc | |
| | | ccgaggtccagttcaactggtacgtggatggcgtggaggtgcataat | |
| | | gccaagacaaagccgcgggaggagcagttcaacagcacgtaccgtgt | |
| | | ggtcagcgtcctcaccgtcctgcaccaggactggctgaacggcaagg | |
| | | agtacaagtgcaaggtctccaacaaaggcctcccgtcctccatcgag | |
| | | aaaaccatctccaaagccaaagggcagccccgagagccacaggtgta | |
| | | caccctgcccccatcccaggaggagatgaccaagaaccaggtcagcc | |
| | | tgacctgcctggtcaaaggcttctaccccagcgacatcgccgtggag | |
| | | tgggaaagcaatgggcagccggagaacaactacaagaccacgcctcc | |
| | | cgtgctggactccgacggctccttcttcctctacagcaggctaaccg | |
| | | tggacaagagcaggtggcaggaggggaatgtcttctcatgctccgtg | |
| | | atgcatgaggctctgcacaaccactacacacagaagagcctctccct | |
| | | gtctctgggt | |
| Ab95 | MC1 VH- | MEWTWVFLFLLSVTAGVHSQVQLQQSGAELMKPGASVKISCKATGYT | 1834 |
| | 458 | FSNYWIEWVKQRPGHGLEWIGEILPGSDSIKYNENFKGKATFTADTS | |
| | | SNTAYMQLSSLISEDSAVYYCARRGNYPDYWGQGTTLTVSSAKTTPP | |
| | | SVYPLAPGSAAQTNSMVTLGCLVKGYFPEPVTVTWNSGSLSSGVHTF | |
| | | PAVLQSDLYTLSSSVTVPSSTWPSETVICNVAHPASSTKVDKKIVPR | |
| | | DCGCKPCICTVPEVSSVFIFPPKPKDVLTITLTPKVTCVVVDISKDD | |
| | | PEVQFSWFVDDVEVHTAQTQPREEQFNSTFRSVSELPIMHQDWLNGK | |
| | | EFKCRVNSAAFPAPIEKTISKTKGRPKAPQVYTIPPPKEQMAKDKVS | |
| | | LTCMITDFFPEDITVEWQWNGQPAENYKNTQPIMDTDGSYFVYSKLN | |
| | | VQKSNWEAGNTFTCSVLHEGLHNHHTEKSLSHSPG | |
| Ab96 | MC1 VH- | EVQLVQSGAEVKKPGESLKISCKGSGYTFSNYWIEWVRQMPGKGLEW | 1835 |
| | 442 | MGEILPGSDSIKYEKNFKGQVTISADKSISTAYLQWSSLKASDTAMY | |
| | | YCARRGNYVDDWGQGTLVTVSSASTKGPSVFPLAPCSRSTSESTAAL | |
| | | GCLVKDYFPEPVTVSWNSGALTSGVHTFPAVLQSSGLYSLSSVVTVP | |
| | | SSSLGTKTYTCNVDHKPSNTKVDKRVESKYGPPCPPCPAPEAAGGPS | |
| | | VFLFPPKPKDTLMISRTPEVTCVVVDVSQEDPEVQFNWYVDGVEVHN | |
| | | AKTKPREEQFNSTYRVVSVLIVLHQDWLNGKEYKCKVSNKGLPSSIE | |
| | | KTISKAKGQPREPQVYTLPPSQEEMTKNQVSLTCLVKGFYPSDIAVE | |
| | | WESNGQPENNYKTTPPVLDSDGSFFLYSRLTVDKSRWQEGNVFSCSV | |
| | | MHEALHNHYTQKSLSLSLG | |
| Ab97 | MC1 VH- | EVQLVQSGAEVKKPGESLKISCKGSGYTFSNYWIEWVRQMPGKGLEW | 1836 |
| | 116 | MGEILPGSDSIKYEKNEKGQVTISADKSISTAYLQWSSLKASDTAMY | |
| | | YCARRGNYVDDWGQGTLVTVSS | |
| Ab98 | PHF1 VH- | gaggtccagctgcaacagtctggacctgagctggtgaagcctggggc | 1837 |
| | 1320 | ttcagtgaagatatcctgcaagacttctggatacacattcactgaat | |
| | | acaccatacactgggtgaagcagagccatggagagagccttgagtgg | |
| | | attggaggtattaatccaaacgatggtggtactatttacaaccagaa | |
| | | gttcaagggcaaggccacattgactgtagacaagtcctccaaaacag | |
| | | cctacatggagctccgcagcctgacatctgaggattctgcagtcttt | |
| | | tactgtgcaagagggccctccgccaggtttccttactggggccaagg | |
| | | gactctggtcactgtctctgcagccaaaacgacacccccatctgtct | |
| | | atccactggcccctggatctgctgcccaaactaactccatggtgacc | |
| | | ctgggatgcctggtcaagggctatttccctgagccagtgacagtgac | |
| | | ctggaactctggatccctgtccagcggtgtgcacaccttcccagctg | |
| | | tcctgcagtctgacctctacactctgagcagctcagtgactgtcccc | |
| | | tccagcacctggcccagcgagaccgtcacctgcaacgttgcccaccc | |
| | | ggccagcagcaccaaggtggacaagaaaattgtgcccagggattgtg | |
| | | gttgtaagccttgcatatgtacagtcccagaagtatcatctgtcttc | |
| | | atcttccccccaaagcccaaggatgtgctcaccattactctgactcc | |
| | | taaggtcacgtgtgttgtggtagacatcagcaaggatgatcccgagg | |
| | | tccagttcagctggtttgtagatgatgtggaggtgcacacagctcag | |
| | | acgcaaccccgggaggagcagttcaacagcactttccgctcagtcag | |
| | | tgaacttcccatcatgcaccaggactggctcaatggcaaggagttca | |
| | | aatgcagggtcaacagtgcagctttccctgcccccatcgagaaaacc | |
| | | atctccaaaaccaaaggcagaccgaaggctccacaggtgtacaccat | |
| | | tccacctcccaaggagcagatggccaaggataaagtcagtctgacct | |
| | | gcatgataacagacttcttccctgaagacattactgtggagtggcag | |
| | | tggaatgggcagccagcggagaactacaagaacactcagcccatcat | |
| | | ggacacagatggctcttacttcgtctacagcaagctcaatgtgcaga | |
| | | agagcaactgggaggcaggaaatactttcacctgctctgtgttacat | |
| | | gagggcctgcacaaccaccatactgagaagagcctctcccactctcc | |
| | | tggt | |
| Ab99 | PHF1 VH- | atgggatggagctggatctttctctttctcctgtcaggaactacagg | 1838 |
| | 1377 | tgtcctctctgaggtccagctgcaacagtctggacctgagctggtga | |
| | | agcctggggcttcagtgaagatatcctgcaagacttctggatacaca | |
| | | ttcactgaatacaccatacactgggtgaagcagagccatggagagag | |
| | | ccttgagtggattggaggtattaatccaaacgatggtggtactattt | |
| | | acaaccagaagttcaagggcaaggccacattgactgtagacaagtcc | |
| | | tccaaaacagcctacatggagctccgcagcctgacatctgaggattc | |
| | | tgcagtcttttactgtgcaagagggccctccgccaggtttccttact | |
| | | ggggccaagggactctggtcactgtctctgcagccaaaacgacaccc | |
| | | ccatctgtctatccactggcccctggatctgctgcccaaactaactc | |
| | | catggtgaccctgggatgcctggtcaagggctatttccctgagccag | |
| | | tgacagtgacctggaactctggatccctgtccagcggtgtgcacacc | |
| | | ttcccagctgtcctgcagtctgacctctacactctgagcagctcagt | |
| | | gactgtcccctccagcacctggcccagcgagaccgtcacctgcaacg | |
| | | ttgcccacccggccagcagcaccaaggtggacaagaaaattgtgccc | |
| | | agggattgtggttgtaagccttgcatatgtacagtcccagaagtatc | |
| | | atctgtcttcatcttccccccaaagcccaaggatgtgctcaccatta | |
| | | ctctgactcctaaggtcacgtgtgttgtggtagacatcagcaaggat | |
| | | gatcccgaggtccagttcagctggtttgtagatgatgtggaggtgca | |
| | | cacagctcagacgcaaccccgggaggagcagttcaacagcactttcc | |
| | | gctcagtcagtgaacttcccatcatgcaccaggactggctcaatggc | |
| | | aaggagttcaaatgcagggtcaacagtgcagctttccctgcccccat | |
| | | cgagaaaaccatctccaaaaccaaaggcagaccgaaggctccacagg | |
| | | tgtacaccattccacctcccaaggagcagatggccaaggataaagtc | |
| | | agtctgacctgcatgataacagacttcttccctgaagacattactgt | |
| | | ggagtggcagtggaatgggcagccagcggagaactacaagaacactc | |
| | | agcccatcatggacacagatggctcttacttcgtctacagcaagctc | |
| | | aatgtgcagaagagcaactgggaggcaggaaatactttcacctgctc | |
| | | tgtgttacatgagggcctgcacaaccaccatactgagaagagcctct | |
| | | cccactctcctggt | |
| Ab100 | PHF1 VH- | gaggtccagctgcaacagtctggacctgagctggtgaagcctggggc | 1839 |
| | 351 | ttcagtgaagatatcctgcaagacttctggatacacattcactgaat | |
| | | acaccatacactgggtgaagcagagccatggagagagccttgagtgg | |
| | | attggaggtattaatccaaacgatggtggtactatttacaaccagaa | |
| | | gttcaagggcaaggccacattgactgtagacaagtcctccaaaacag | |
| | | cctacatggagctccgcagcctgacatctgaggattctgcagtcttt | |
| | | tactgtgcaagagggccctccgccaggtttccttactggggccaagg | |
| | | gactctggtcactgtctctgca | |
| Ab101 | PHF1 VH- | atgggatggagctggatctttctctttctcctgtcaggaactacagg | 1840 |
| | 699 | tgtcctctctgaggtccagctgcaacagtctggacctgagctggtga | |
| | | agcctggggcttcagtgaagatatcctgcaagacttctggatacaca | |
| | | ttcactgaatacaccatacactgggtgaagcagagccatggagagag | |
| | | ccttgagtggattggaggtattaatccaaacgatggtggtactattt | |
| | | acaaccagaagttcaagggcaaggccacattgactgtagacaagtcc | |
| | | tccaaaacagcctacatggagctccgcagcctgacatctgaggattc | |
| | | tgcagtcttttactgtgcaagagggccctccgccaggtttccttact | |
| | | ggggccaagggactctggtcactgtctctgcagccaaaacgacaccc | |
| | | ccatctgtctatccactggcccctggatctgctgcccaaactaactc | |
| | | catggtgaccctgggatgcctggtcaagggctatttccctgagccag | |
| | | tgacagtgacctggaactctggatccctgtccagcggtgtgcacacc | |
| | | ttcccagctgtcctgcagtctgacctctacactctgagcagctcagt | |
| | | gactgtcccctccagcacctggcccagcgagaccgtcacctgcaacg | |
| | | ttgcccacccggccagcagcaccaaggggacaagaaaatt | |
| Ab102 | PHF1 VH- | atgggatggagctggatctttctctttctcctgtcaggaactacagg | 1841 |
| | 717 | tgtcctctctgaggtccagctgcaacagtctggacctgagctggtga | |
| | | agcctggggcttcagtgaagatatcctgcaagacttctggatacaca | |
| | | ttcactgaatacaccatacactgggtgaagcagagccatggagagag | |
| | | ccttgagtggattggaggtattaatccaaacgatggtggtactattt | |
| | | acaaccagaagttcaagggcaaggccacattgactgtagacaagtcc | |
| | | tccaaaacagcctacatggagctccgcagcctgacatctgaggattc | |
| | | tgcagtcttttactgtgcaagagggccctccgccaggtttccttact | |
| | | ggggccaagggactctggtcactgtctctgcagccaaaacgacaccc | |
| | | ccatctgtctatccactggcccctggatctgctgcccaaactaactc | |
| | | catggtgaccctgggatgcctggtcaagggctatttccctgagccag | |
| | | tgacagtgacctggaactctggatccctgtccagcggtgtgcacacc | |
| | | ttcccagctgtcctgcagtctgacctctacactctgagcagctcagt | |
| | | gactgtcccctccagcacctggcccagcgagaccgtcacctgcaacg | |
| | | ttgcccacccggccagcagcaccaaggtggacaagaaaattgtgccc | |
| | | agggattgtggt | |
| Ab103 | PHF1 VH- | atgggatggagctggatctttctctttctcctgtcaggaactacagg | 1842 |
| | 744 | tgtcctctctgaggtccagctgcaacagtctggacctgagctggtga | |
| | | agcctggggcttcagtgaagatatcctgcaagacttctggatacaca | |
| | | ttcactgaatacaccatacactgggtgaagcagagccatggagagag | |
| | | ccttgagtggattggaggtattaatccaaacgatggtggtactattt | |
| | | acaaccagaagttcaagggcaaggccacattgactgtagacaagtcc | |
| | | tccaaaacagcctacatggagctccgcagcctgacatctgaggattc | |
| | | tgcagtcttttactgtgcaagagggccctccgccaggtttccttact | |
| | | ggggccaagggactctggtcactgtctctgcagccaaaacgacaccc | |
| | | ccatctgtctatccactggcccctggatctgctgcccaaactaactc | |
| | | catggtgaccctgggatgcctggtcaagggctatttccctgagccag | |
| | | tgacagtgacctggaactctggatccctgtccagcggtgtgcacacc | |
| | | ttcccagctgtcctgcagtctgacctctacactctgagcagctcagt | |
| | | gactgtcccctccagcacctggcccagcgagaccgtcacctgcaacg | |
| | | ttgcccacccggccagcagcaccaaggtggacaagaaaattgtgccc | |
| | | agggattgtggttgtaagccttgcatatgtacagtccca | |
| Ab104 | PHF1 VH- | atgggatggagctggatctttctctttctcctgtcaggaactacagg | 1843 |
| | 753 | tgtcctctctgaggtccagctgcaacagtctggacctgagctggtga | |
| | | agcctggggcttcagtgaagatatcctgcaagacttctggatacaca | |
| | | ttcactgaatacaccatacactgggtgaagcagagccatggagagag | |
| | | ccttgagtggattggaggtattaatccaaacgatggtggtactattt | |
| | | acaaccagaagttcaagggcaaggccacattgactgtagacaagtcc | |
| | | tccaaaacagcctacatggagctccgcagcctgacatctgaggattc | |
| | | tgcagtcttttactgtgcaagagggccctccgccaggtttccttact | |
| | | ggggccaagggactctggtcactgtctctgcagccaaaacgacaccc | |
| | | ccatctgtctatccactggcccctggatctgctgcccaaactaactc | |
| | | catggtgaccctgggatgcctggtcaagggctatttccctgagccag | |
| | | tgacagtgacctggaactctggatccctgtccagcggtgtgcacacc | |
| | | ttcccagctgtcctgcagtctgacctctacactctgagcagctcagt | |
| | | gactgtcccctccagcacctggcccagcgagaccgtcacctgcaacg | |
| | | ttgcccacccggccagcagcaccaaggtggacaagaaaattgtgccc | |
| | | agggattgtggttgtaagccttgcatatgtacagtcccagaagtatc | |
| | | a | |
| Ab105 | PHF1 VH- | MGWSWIFLFLLSGTTGVLSEVQLQQSGPELVKPGASVKISCKTSGYT | 1844 |
| | 459 | FTEYTIHWVKQSHGESLEWIGGINPNDGGTIYNQKFKGKATLTVDKS | |
| | | SKTAYMELRSLISEDSAVFYCARGPSARFPYWGQGTLVTVSAAKTTP | |
| | | PSVYPLAPGSAAQTNSMVTLGCLVKGYFPEPVTVTWNSGSLSSGVHT | |
| | | FPAVLQSDLYTLSSSVTVPSSTWPSETVTCNVAHPASSTKVDKKIVP | |
| | | RDCGCKPCICTVPEVSSVFIFPPKPKDVLTITLTPKVTCVVVDISKD | |
| | | DPEVQFSWFVDDVEVHTAQTQPREEQFNSTFRSVSELPIMHQDWLNG | |
| | | KEFKCRVNSAAFPAPIEKTISKTKGRPKAPQVYTIPPPKEQMAKDKV | |
| | | SLTCMITDFFPEDITVEWQWNGQPAENYKNTQPIMDTDGSYFVYSKL | |
| | | NVQKSNWEAGNTFTCSVLHEGLHNHHTEKSLSHSPG | |
| Ab106 | PHF1 VH | atgggatggagctggatctttctctttctcctgtcaggaactacagg | 1845 |
| | + | tgtcctctctgctgccgaggtccagctgcaacagtctggacctgagc | |
| | | tggtgaagcctggggcttcagtgaagatatcctgcaagacttctgga | |
| | | tacacattcactgaatacaccatacactgggtgaagcagagccatgg | |
| | | agagagccttgagtggattggaggtattaatccaaacgatggtggta | |
| | | ctatttacaaccagaagttcaagggcaaggccacattgactgtagac | |
| | | aagtcctccaaaacagcctacatggagctccgcagcctgacatctga | |
| | | ggattctgcagtcttttactgtgcaagagggccctccgccaggtttc | |
| | | cttactggggccaagggactctggtcactgtctctgcagccaaaacg | |
| | | acacccccatctgtctatccactggcccctggatctgctgcccaaac | |
| | | taactccatggtgaccctgggatgcctggtcaagggctatttccctg | |
| | | agccagtgacagtgacctggaactctggatccctgtccagcggtgtg | |
| | | cacaccttcccagctgtcctgcagtctgacctctacactctgagcag | |
| | | ctcagtgactgtcccctccagcacctggcccagcgagaccgtcacct | |
| | | gcaacgttgcccacccggccagcagcaccaaggtggacaagaaaatt | |
| | | gtgcccagggattgtggttgtaagccttgcatatgtacagtcccaga | |
| | | agtatcatctgtcttcatcttccccccaaagcccaaggatgtgctca | |
| | | ccattactctgactcctaaggtcacgtgtgttgtggtagacatcagc | |
| | | aaggatgatcccgaggtccagttcagctggtttgtagatgatgtgga | |
| | | ggtgcacacagctcagacgcaaccccgggaggagcagttcaacagca | |
| | | ctttccgctcagtcagtgaacttcccatcatgcaccaggactggctc | |
| | | aatggcaaggagttcaaatgcagggtcaacagtgcagctttccctgc | |
| | | ccccatcgagaaaaccatctccaaaaccaaaggcagaccgaaggctc | |
| | | cacaggtgtacaccattccacctcccaaggagcagatggccaaggat | |
| | | aaagtcagtctgacctgcatgataacagacttcttccctgaagacat | |
| | | tactgtggagtggcagtggaatgggcagccagcggagaactacaaga | |
| | | acactcagcccatcatggacacagatggctcttacttcgtctacagc | |
| | | aagctcaatgtgcagaagagcaactgggaggcaggaaatactttcac | |
| | | ctgctctgtgttacatgagggcctgcacaaccaccatactgagaaga | |
| | | gcctctcccactctcctggt | |
| Ab107 | PT1 VH- | caggttcagctgcagcagtctggaactgagctgatgaagcctggggc | 1846 |
| | 357 | ctcagtgaagatatcctgcaaggctactggctacacattcagtagct | |
| | | cctggatggggtgggttaagcagaggcctggacatggccttgagtgg | |
| | | attggagacattttacctggaagtggtggtactaactacaatgagag | |
| | | gttcaagggcaaggcctcattcactgcagaaacatcctccaacacag | |
| | | cctacatgcaactcagcagcctgacatctgaggactctgccgtctat | |
| | | tactgtgtaagaagctactatgattacgaccgctttgctaactgggg | |
| | | ccaagggactctggtcactgtctctgca | |
| Ab108 | PT1 VH- | QVQLQQSGTELMKPGASVKISCKATGYTFSSSWMGWVKQRPGHGLEW | 1847 |
| | 119 | IGDILPGSGGTNYNERFKGKASFTAETSSNTAYMQLSSLTSEDSAVY | |
| | | YCVRSYYDYDRFANWGQGTLVTVSA | |
| Ab109 | PT3 VH- | gaagtgaagctggtggagtctgggggagacttagtgaagcctggagg | 1848 |
| | 354 | gtccctgaaactctcctgtgcagcctctggattcactttcagtagct | |
| | | atgccatgtcttgggttcgccagaatccagagaagaggctggagtgg | |
| | | gtcgcatccattagtaagggtggtaacacctactatccaaacagcgt | |
| | | gaagggccgattcaccatctccagagataatgccaggaacatcctgt | |
| | | acctgcaaatgagcagtctgaggtctgaggacacggccctttattac | |
| | | tgtgcaagaggctggggtgattacgggtggtttgcttactggggcca | |
| | | agtgactctggtcactgtctctgca | |
| Ab110 | PT3 VH- | EVKLVESGGDLVKPGGSLKLSCAASGFTFSSYAMSWVRQNPEKRLEW | 1849 |
| | 118 | VASISKGGNTYYPNSVKGRFTISRDNARNILYLQMSSLRSEDTALYY | |
| | | CARGWGDYGWFAYWGQVTLVTVSA | |
| Ab111 | PT3 VH | gaagtgaaactggtggaaagcggcggcgatctggtgaaaccgggcgg | 1850 |
| | Bioinformatics | cagcctgaaactgagctgcgcggcgagcggctttacctttagcagct | |
| | | atgcgatgagctgggtgcgccagaacccggaaaaacgcctggaatgg | |
| | | gtggcgagcattagcaaaggcggcaacacctattatccgaacagcgt | |
| | | gaaaggccgctttaccattagccgcgataacgcgcgcaacattctgt | |
| | | atctgcagatgagcagcctgcgcagcgaagataccgcgctgtattat | |
| | | tgcgcgcgcggctggggcgattatggctggtttgcgtattggggcca | |
| | | ggtgaccctggtgaccgtgagcgcg | |
| Ab112 | PT3 VH | gaggtgaagctggtggagagcggaggagacctggtcaagcccggggg | 1851 |
| | Bioinformatics | ctccctgaagctcagctgtgccgccagcggcttcaccttctcctcct | |
| | 2 | atgccatgtcctgggtccggcagaaccctgagaagaggctggagtgg | |
| | | gtggcctccatctccaagggcggcaacacctactatcccaacagcgt | |
| | | gaaaggaagatttaccatcagccgggacaatgccaggaacatcctct | |
| | | acctgcagatgagcagcctgcggtctgaggacacagccctgtactac | |
| | | tgcgcccggggctggggggactatggatggtttgcctactggggcca | |
| | | ggtgaccctggttactgtgtctgcc | |
| Ab113 | PT3 VH | gaggtgaagctggtggagagcggcggcgacctggtgaagcccggcgg | 1852 |
| | EMBOSS | cagcctgaagctgagctgcgccgccagcggcttcaccttcagcagct | |
| | | acgccatgagctgggtgaggcagaaccccgagaagaggctggagtgg | |
| | | gtggccagcatcagcaagggcggcaacacctactaccccaacagcgt | |
| | | gaagggcaggttcaccatcagcagggacaacgccaggaacatcctgt | |
| | | acctgcagatgagcagcctgaggagcgaggacaccgccctgtactac | |
| | | tgcgccaggggctggggcgactacggctggttcgcctactggggcca | |
| | | ggtgaccctggtgaccgtgagcgcc | |
| Ab114 | PT3 VH | gaggtgaaacttgtggagagcggcggggacctggtcaagccaggcgg | 1853 |
| | Geneinfinity | cagcctgaagctgagctgtgccgcctccggcttcaccttcagcagct | |
| | | atgccatgtcctgggtccggcagaaccctgagaagaggctggagtgg | |
| | | gtggccagcatcagcaaaggaggcaacacctactatcccaacagcgt | |
| | | gaagggccgcttcaccatctcccgggacaatgccaggaacatcctct | |
| | | acctgcagatgagctccctgcggtctgaggacacagccctgtactac | |
| | | tgcgcccggggctggggagactatggatggtttgcctactggggcca | |
| | | ggtgaccctggtgacagtgtctgcc | |
| Ab115 | PT3 VH | gaggtgaagctggtggagtctggcggagacctggtgaagccaggcgg | 1854 |
| | Genescript | aagcctgaagctgtcctgcgccgcttctggcttcacctttagctcct | |
| | | acgccatgagctgggtgcggcagaacccagagaagcgcctggagtgg | |
| | | gtggcttccatctctaagggcggaaacacctactaccctaacagcgt | |
| | | gaagggaaggttcacaatctccagggataacgccagaaacatcctgt | |
| | | acctgcagatgtctagcctgaggtccgaggacacagccctgtactac | |
| | | tgtgctcggggctggggagattacggctggtttgcctactggggaca | |
| | | ggtgaccctggtgacagtgtctgct | |
| Ab116 | PT3 VH | gaggtgaaacttgtggagagcggcggggacctggtgaagccaggcgg | 1855 |
| | IDT | cagcctgaagctgagctgtgccgcctccggcttcaccttcagcagct | |
| | | atgccatgtcctgggtccggcagaaccctgagaagaggctggagtgg | |
| | | gtggccagcatcagcaaaggaggcaacacctactaccccaacagcgt | |
| | | caagggccgcttcaccatctcccgggacaatgccaggaacatcctgt | |
| | | acctgcagatgagctccctgcggtctgaggacacagccctgtattac | |
| | | tgcgcccggggctggggagactatggatggtttgcctactggggcca | |
| | | ggtgaccctggtcaccgtgtctgcc | |
| Ab117 | PT3 VH | gaggtgaaacttgtggagtctggaggagacctggtgaagccaggagg | 1856 |
| | NUS | cagcctgaagctgagctgtgctgccagtggcttcaccttcagcagct | |
| | | atgccatgtcctgggtccggcagaaccctgagaagaggctggagtgg | |
| | | gtggccagcatctccaagggcggcaacacctactatcccaacagcgt | |
| | | caaaggaagatttaccatcagccgggacaatgccaggaacatcctct | |
| | | acctgcagatgagctccctgcggtctgaggacacagccctgtactac | |
| | | tgtgcccggggctggggagattatggatggtttgcctactggggcca | |
| | | ggtgaccctggtcactgtgtctgct | |
| Ab118 | PT3 VH | gaggtgaaattagtggagtctggaggagacctggtgaagccaggagg | 1857 |
| | NUS2 | cagcctgaagctgagctgtgctgcttctggcttcaccttcagcagct | |
| | | atgccatgtcctgggtcagacagaatcctgagaagagactggagtgg | |
| | | gtggccagcatctccaaaggtggcaacacctactatcccaacagtgt | |
| | | taaaggaagatttaccatcagcagagataatgcaagaaacatcctct | |
| | | acctgcagatgtcttctttaagaagtgaagatacagccctgtactac | |
| | | tgtgccagaggctggggagattatggatggtttgcctactggggcca | |
| | | ggtgacattagttactgtgtctgct | |
| Ab119 | PT3 VH | gaggtgaagctggtggagagcggcggggacctggtcaagcccggggg | 1858 |
| | NUS3 | cagcctgaagctcagctgtgccgcctccggcttcaccttcagcagct | |
| | | atgccatgtcctgggtccggcagaaccctgagaagaggctggagtgg | |
| | | gtggccagcatcagcaagggcggcaacacctactatcccaacagcgt | |
| | | gaaaggccgcttcaccatctcccgggacaatgccaggaacatcctgt | |
| | | acctgcagatgagctccctgcgcagcgaggacacagccctctactac | |
| | | tgcgcccggggctggggagactatggatggtttgcctactggggcca | |
| | | ggtgaccctggttactgtgtctgcc | |
| Ab120 | PT3 VH | gaggtgaagctggtggagagcggcggcgacctggtgaagcctggcgg | 1859 |
| | Snapgene | cagcctgaagctgagctgcgccgccagcggcttcaccttcagcagct | |
| | | acgccatgagctgggtgagacagaaccctgagaagagactggagtgg | |
| | | gtggccagcatcagcaagggcggcaacacctactaccctaacagcgt | |
| | | gaagggcagattcaccatcagcagagacaacgccagaaacatcctgt | |
| | | acctgcagatgagcagcctgagaagcgaggacaccgccctgtactac | |
| | | tgcgccagaggctggggcgactacggctggttcgcctactggggcca | |
| | | ggtgaccctggtgaccgtgagcgcc | |
| Ab121 | VH | EVHLVESGGALVKPGGSLRLSCAASGFSFS | 1860 |
| Ab122 | IPN002 | atgaagttgcctgttaggctgttggtgctgatgttctggattcctgc | 1861 |
| | (VL | ttccagcagt | |
| | Signal) | | |
| Ab123 | PHF1 (VL | atgagtcctgcccagttcctgtttctgttagtgctctggattcggga | 1862 |
| | Signal) | aaccaacggt | |
| Ab124 | PHF1 (VL | gccgccaccatgagtcctgcccagttcctgtttctgttagtgctctg | 1863 |
| | Signal 72) | gattcgggaaaccaacggtgctgcc | |
| Ab125 | VL | cgggctgatgctgcaccaactgtatccatcttcccaccatccagtga | 1864 |
| | (constant) | gcagttaacatctggaggtgcctcagtcgtgtgcttcttgaacaact | |
| | | tctaccccaaagacatcaatgtcaagtggaagattgatggcagtgaa | |
| | | cgacaaaatggcgtcctgaacagttggactgatcaggacagcaaaga | |
| | | cagcacctacagcatgagcagcaccctcacgttgaccaaggacgagt | |
| | | atgaacgacataacagctatacctgtgaggccactcacaagacatca | |
| | | acttcacccattgtcaagagcttcaacaggaatgagtgt | |
| Ab126 | 7295-M6 | RSSQTIVHSNGNTYLE | 1865 |
| | LCDR | | |
| Ab127 | 7295-M6 | FQGSHVPFT | 1866 |
| | LCDR | | |
| Ab128 | 7297-2M1 | KASQDINKYIA | 1867 |
| | LCDR | | |
| Ab129 | 7297-2M1 | YTSTLQS | 1868 |
| | LCDR | | |
| Ab130 | 7297-2M1 | LQYDHLLT | 1869 |
| | LCDR | | |
| Ab131 | 7298-M1 | RASKSVSTSDYSYMH | 1870 |
| | LCDR | | |
| Ab132 | 7298-M1 | LASNLES | 1871 |
| | LCDR | | |
| Ab133 | 7298-M1 | QHSRELPFT | 1872 |
| | LCDR | | |
| Ab134 | 7299-M2 | KSSQSLLYSDGKTFLN | 1873 |
| | LCDR | | |
| Ab135 | 7299-M2 | LVSKLQS | 1874 |
| | LCDR | | |
| Ab136 | 7299-M2 | VQGTHFPYT | 1875 |
| | LCDR | | |
| Ab137 | 7299-M5 | KSSQSLLYSDGKTYLN | 1876 |
| | LCDR | | |
| Ab138 | 7299-M5 | QVSKLDP | 1877 |
| | LCDR | | |
| Ab139 | 7299-M5 | LQGTYYPHT | 1878 |
| | LCDR | | |
| Ab140 | 7299-M9 | LQGTYYPH | 1879 |
| | LCDR | | |
| Ab141 | C10.2 | QASQGTSINLN | 1880 |
| | LCDR1 | | |
| Ab142 | C10.2 | GASNLED | 1881 |
| | LCDR2 | | |
| Ab143 | C10.2 | LQHTYLP | 1882 |
| | LCDR3 | | |
| Ab144 | C5.2 | QASQDTSINLN | 1883 |
| | LCDR1 | | |
| Ab145 | C8.3 | GSSNLED | 1884 |
| | LCDR2 | | |
| Ab146 | C8.3 | LQHSYLP | 1885 |
| | LCDR3 | | |
| Ab147 | D1.2 | RSSQSLVHSNGNTYLH | 1886 |
| | LCDR1 | | |
| Ab148 | D1.2 | KVSNRFS | 1887 |
| | LCDR2 | | |
| Ab149 | D1.2 | SQSTHVP | 1888 |
| | LCDR3 | | |
| Ab150 | IPN002 | WYLQKPGQSPQLLXY | 1889 |
| | LCDR1 | | |
| Ab151 | IPN002 | GVPDRFSGSGSGTDFTLKISRVEAEDVGXYYC | 1890 |
| | LCDR2 | | |
| Ab152 | IPN002 | FGGGTKVEIK | 1891 |
| | LCDR3 | | |
| Ab153 | LCDR1 | RSSQTILHSNGNTYLE | 1892 |
| Ab154 | LCDR1 | KSSQSIVHSNGNTYLE | 1893 |
| Ab155 | LCDR1 | WYLQKPGQSPQLLVY | 1894 |
| Ab156 | LCDR2 | KVSKRFS | 1895 |
| Ab157 | LCDR2 | GVPDRFSGSGSGTDFTLKISRVEAEDVGTYYC | 1896 |
| Ab158 | LCDR3 | FQGSLVPWA | 1897 |
| Ab159 | MC1 | RSSQSLVHSNQNTYLH | 1898 |
| | LCDR1 | | |
| Ab160 | MC1 | YKVDNRFS | 1899 |
| | LCDR2 | | |
| Ab161 | MC1 | SQSTLVPLT | 1900 |
| | LCDR3 | | |
| Ab162 | PT1 | SESLLHSNGNTY | 1901 |
| | LCDR1 | | |
| | (Chothia) | | |
| Ab163 | PT1 | RSSESLLHSNGNTYLY | 1902 |
| | LCDR1 | | |
| | (Kabat) | | |
| Ab164 | PT1 | RMS | 1903 |
| | LCDR2 | | |
| | (Chothia) | | |
| Ab165 | PT1 | RMSNLAS | 1904 |
| | LCDR2 | | |
| | (Kabat) | | |
| Ab166 | PT1 | YLEYPL | 1905 |
| | LCDR3 | | |
| | (Chothia) | | |
| Ab167 | PT1 | MQYLEYPLT | 1906 |
| | LCDR3 | | |
| | (Kabat) | | |
| Ab168 | PT3 | SQDINRY | 1907 |
| | LCDR1 | | |
| | (Chothia) | | |
| Ab169 | PT3 | KASQDINRYLN | 1908 |
| | LCDR1 | | |
| | (Kabat) | | |
| Ab170 | PT3 | RAN | 1909 |
| | LCDR2 | | |
| | (Chothia) | | |
| Ab171 | PT3 | RANRLLD | 1910 |
| | LCDR2 | | |
| | (Kabat) | | |
| Ab172 | PT3 | YDEFPL | 1911 |
| | LCDR3 | | |
| | (Chothia) | | |
| Ab173 | PT3 | LQYDEFPLT | 1912 |
| | LCDR3 | | |
| | (Kabat) | | |
| Ab174 | 7295-M6 | DIVLTQTPLSLPVSLGDPASISCRSSQTIVHSNGNTYLEWYLQKPGQ | 1913 |
| | VL | SPKLLIYKVSNRFSGVPDRFSGSGSGTDFTLKISRVEAEDLGVYYCF | |
| | | QGSHVPFTFGSGTKLEIKRADAAPTVST | |
| Ab175 | 7297-2M1 | DIVLTQTPSSLSASLGGKVTITCKASQDINKYIAWYQHKPGKGPRLL | 1914 |
| | VL | IHYTSTLQSGIPSRFSGSGSGRDYSFSISNLEPEDIATYYCLQYDHL | |
| | | LIFGAGTKLELKRADAAPTVST | |
| Ab176 | 7298-M1 | DIVLIQSPTSLAVSLGQRATISCRASKSVSTSDYSYMHWYQQKPGQP | 1915 |
| | VL | PKLLIYLASNLESGVPARFSGSGSGTDFTLNIHPVEEEDAATYYCQH | |
| | | SRELPFTFGGGTKLEIKRADAAPTVST | |
| Ab177 | 7299-M2 | DIVLTQTPLTLSVTIGQPASISCKSSQSLLYSDGKTFLNWLLQSPGQ | 1916 |
| | VL | SPRLLIYLVSKLQSGVPDRFSGSGSGTDFTLKISRVEAEDLGVYYCV | |
| | | QGTHEPYTFGGGTKLEIKRADAAPTVST | |
| Ab178 | 7299-M5 | DIVMTQSPLSLSVTIGQPASISCKSSQSLLYSDGKTYLNWLQQRPGQ | 1917 |
| | VL | SPKRLVYQVSKLDPGIPDRFSGSGSETDFTLKLSRVEAEDLGVYYCL | |
| | | QGTYYPHTFGGGTKLEIKRADAAPTVST | |
| Ab179 | 7299-M9 | DIVITQSPLSLSVTIGQPASISCKSSQSLLYSDGKTYLNWLQQRPGQ | 1918 |
| | VL | SPKRLVYQVSKLDPGIPDRESGSGSETDFTLKLSRVEAEDLGVYYCL | |
| | | QGTYYPHTFGGGTKLEIKRADAAPTVST | |
| Ab180 | C10.2 VL- | DVQMIQSPSSLSASLGDIVTMTCQASQGISINLNWFQQKPGKAPKLL | 1919 |
| | 107 | IYGASNLEDGVPSRFSGSRYGTDFTLTISSLEDEDMATYFCLQHTYL | |
| | | PFTFGSGTKLEIK | |
| Ab181 | C10.2 VL- | DVQMIQSPSSLSASLGDIVTMTCQASQGISINLNWFQQKPGKAPKLL | 1920 |
| | 214 | IYGASNLEDGVPSRFSGSRYGTDFTLTISSLEDEDMATYFCLQHTYL | |
| | | PFTFGSGTKLEIKRADAAPTVSIFPPSSEQLTSGGASVVCFLNNFYP | |
| | | KDINVKWKIDGSERQNGVLNSWTDQDSKDSTYSMSSTLTLTKDEYER | |
| | | HNSYTCEATHKTSTSPIVKSFNRNEC | |
| Ab182 | C10.2 VL | gacgtgcagatgatccagagccccagcagcctgagcgccagcctggg | 1921 |
| | Bioinformatics | cgacatcgtgaccatgacctgccaggccagccagggcaccagcatca | |
| | | acctgaactggttccagcagaagcccggcaaggcccccaagctgctg | |
| | | atctacggcgccagcaacctggaggacggcgtgcccagcaggttcag | |
| | | cggcagcaggtacggcaccgacttcaccctgaccatcagcagcctgg | |
| | | aggacgaggacatggccacctacttctgcctgcagcacacctacctg | |
| | | cccttcaccttcggcagcggcaccaagctggagatcaag | |
| Ab183 | C10.2 VL | gacgttcagatgatccagagccccagcagcctgagcgccagcctggg | 1922 |
| | EMBOSS | cgacatcgtgaccatgacctgccaggccagccagggcaccagcatca | |
| | | acctgaactggttccagcagaagcccggcaaggcccccaagctgctg | |
| | | atctacggcgccagcaacctggaggacggcgtgcccagccggttcag | |
| | | cggcagccggtacggcaccgacttcaccctgaccatcagcagcctgg | |
| | | aggacgaggacatggccacctacttctgcctgcagcacacctacctg | |
| | | cccttcaccttcggcagcggcaccaagctggagatcaag | |
| Ab184 | C10.2 VL | gatgtgcagatgatccagagcccttcttccctctccgcctccctggg | 1923 |
| | GeneInfinity | cgacatcgtgaccatgacctgccaggccagccagggcaccagcatca | |
| | | acctgaactggttccagcagaagcccgggaaggcccccaagctcctc | |
| | | atctacggcgcctcaaatttggaagatggagtgcccagccgcttctc | |
| | | cgggtcccgctatggaacagacttcacactgaccatcagcagcctgg | |
| | | aggatgaggacatggccacctacttctgcctgcagcacacatacctg | |
| | | cccttcaccttcggcagcgggaccaagctggagatcaag | |
| Ab185 | C10.2 VL | gacgtgcagatgatccagagcccctccagcctgagcgcctcgctggg | 1924 |
| | GregThatcher | ggacatcgtgaccatgacctgccaggcctcccagggcaccagcatca | |
| | | acctcaactggttccagcagaagcccggcaaggcccccaagctgctg | |
| | | atctacggggccagcaacctggaggacggcgtgcccagccgcttcag | |
| | | cggctcccgctacggcacggacttcacgctgaccatctcctccctgg | |
| | | aggacgaggacatggccacctacttctgcctgcagcacacctacctg | |
| | | cccttcaccttcggcagcgggaccaagctggagatcaag | |
| Ab186 | C10.2 VL | gacgtgcagatgatccagagccccagctccctcagcgcctccctggg | 1925 |
| | IDT | ggacatcgtgaccatgacctgccaggccagccagggcaccagcatca | |
| | | acctcaactggttccagcagaagcccggcaaggcccccaagctgctg | |
| | | atctacggggcctccaacctggaggacggcgtgccctcccgcttcag | |
| | | cggcagccgctacggcacggacttcacgctgaccatcagcagcctgg | |
| | | aggacgaggacatggccacctacttctgcctgcagcacacctacctg | |
| | | cccttcaccttcgggagcgggaccaagttggagatcaag | |
| Ab187 | C10.2 VL | gacgtgcagatgatccagagccccagcagcctgagcgcctcgctggg | 1926 |
| | InSilico | ggacatcgtgaccatgacctgccaggcctcccagggcaccagcatca | |
| | | acctcaactggttccagcagaagcccggcaaggcccccaagctcctc | |
| | | atctacggggccagcaacctggaggacggcgtgccctcccgcttcag | |
| | | cggctcccgctacggcacggacttcacgctgaccatctcctccctgg | |
| | | aggacgaggacatggccacctacttctgcctgcagcacacctacctg | |
| | | cccttcaccttcggcagcgggaccaagctggagatcaag | |
| Ab188 | C10.2 VL | gacgttcagatgatccagtccccctcctccctgtccgcctccctggg | 1927 |
| | MolBio | cgacatcgtgaccatgacctgccaggcctcccagggcacctccatca | |
| | | acctgaactggttccagcagaagcccggcaaggcccccaagctgctg | |
| | | atctacggcgcctccaacctggaggacggcgtgccctcccggttctc | |
| | | cggctcccggtacggcaccgacttcaccctgaccatctcctccctgg | |
| | | aggacgaggacatggccacctacttctgcctgcagcacacctacctg | |
| | | cccttcaccttcggctccggcaccaagctggagatcaag | |
| Ab189 | C10.2 VL | gacgtgcagatgatccagagcccctcctccctgagcgccagcctggg | 1928 |
| | N2P | ggacatcgtgaccatgacctgccaggcctcccagggcaccagcatca | |
| | | acctcaactggttccagcagaagcccggcaaggcccccaagctcctc | |
| | | atctacggggccagcaacctggaggacggcgtgcccagccgcttcag | |
| | | cggctcccgctacggcacggacttcacgctgaccatcagcagcctgg | |
| | | aggacgaggacatggccacctacttctgcctgcagcacacctacctg | |
| | | cccttcaccttcggcagcgggaccaagctggagatcaag | |
| Ab190 | C10.2 VL | gacgttcagatgatccagagccctagcagcctgagcgccagcctggg | 1929 |
| | Snapgene | cgacatcgtgaccatgacctgccaggccagccagggcaccagcatca | |
| | | acctgaactggttccagcagaagcctggcaaggcccctaagctgctg | |
| | | atctacggcgccagcaacctggaggacggcgtgcctagccgtttcag | |
| | | cggcagccgttacggcaccgacttcaccctgaccatcagcagcctgg | |
| | | aggacgaggacatggccacctacttctgcctgcagcacacctacctg | |
| | | cctttcaccttcggcagcggcaccaagctggagatcaag | |
| Ab191 | C10.2 VL | gacgttcagatgatccagagccctagcagcctgagcgccagcctggg | 1930 |
| | Vector NTI | cgacatcgtgaccatgacctgccaggccagccagggcaccagcatca | |
| | | acctgaactggttccagcagaagcctggcaaggcccctaagctgctg | |
| | | atctacggcgccagcaacctggaggacggcgtgcccagccggttcag | |
| | | cggcagccggtacggcaccgacttcaccctgaccatcagcagcctgg | |
| | | aggacgaggacatggccacctacttctgcctgcagcacacctacctg | |
| | | cccttcaccttcggcagcggcaccaagctggagatcaag | |
| Ab192 | C5.2 VL | DVQMIQSPSSLSASLGDIVTMTCQASQDTSINLNWFQQKPGKAPKLL | 1931 |
| | | IYGASNLEDGVPSRFSGSRYGTDFTLTISSLEDEDMATYFCLQHTYL | |
| | | PFTFGSGTKLEIKRADAAPTVSIFPPSSEQLTSGGASVVCFLNNFYP | |
| | | KDINVKWKIDGSERQNGVLNSWTDQDSKDSTYSMSSTLTLTKDEYER | |
| | | HNSYTCEATHKTSTSPIVKSFNRNEC | |
| Ab193 | C8.3 VL | DVQMIQSPSSLSASLGDIVTMTCQASQGISINLNWFQQKPGKAPKLL | 1932 |
| | | IYGSSNLEDGVPSRFSGSRYGTDFTLTISSLEDEDMATYFCLQHSYL | |
| | | PFTFGSGTKLEIKRADAAPTVSIFPPSSEQLTSGGASVVCFLNNFYP | |
| | | KDINVKWKIDGSERQNGVLNSWTDQDSKDSTYSMSSTLTLTKDEYER | |
| | | HNSYTCEATHKISTSPIVKSFNRNEC | |
| Ab194 | D1.2 VL | DVMMTQTPLSLPVSLGDQASISCRSSQSLVHSNGNTYLHWHLQKPGQ | 1933 |
| | | SPKFLIYKVSNRFSGVPDRFSGSGSGTDFTLKISRVEAEDLGVYFCS | |
| | | QSTHVPFTFGSGTKLEIKRADAAPTVSIFPPSSEQLTSGGASVVCFL | |
| | | NNFYPKDINVKWKIDGSERQNGVLNSWTDQDSKDSTYSMSSTLTLTK | |
| | | DEYERHNSYTCEATHKTSTSPIVKSFNRNEC | |
| Ab195 | D1.2* VL | DVVMTQTPLSLPVSLGDQASISCRSSQSLVHSNGNTYLHWHLQKPGQ | 1934 |
| | | SPKFLIYKVSNRFSGVPDRFSGSGSGTDFTLKISRVEAEDLGVYFCS | |
| | | QSTHVPFTFGSGTKLEIKRADAAPTVSIFPPSSEQLTSGGASVVCFL | |
| | | NNFYPKDINVKWKIDGSERQNGVLNSWTDQDSKDSTYSMSSTLTLTK | |
| | | DEYERHNSYTCEATHKTSTSPIVKSENRNEC | |
| Ab196 | hC10.2 VL | DVQMTQSPSSLSASVGDRVTMTCQASQDTSINLNWFQQKPGKAPKLL | 1935 |
| | | IYGASNLETGVPSRFSGSRSGTDFTLTISSLQPEDMATYYCLQHTYL | |
| | | PFTFGSGTKLEIKRTVAAPSVFIFPPSDEQLKSGTASVVCLLNNFYP | |
| | | REAKVQWKVDNALQSGNSQESVTEQDSKDSTYSLSSTLTLSKADYEK | |
| | | HKVYACEVTHQGLSSPVTKSFNRGEC | |
| Ab197 | IPN001 VL- | gatgttttgatgacccaaactccgctctccctggcagtcaatcttgg | 1936 |
| | 336 | agatcaagcctccctctcttgcagatcgagtcagactattttacata | |
| | | gtaatggaaatacctatttagaatggtatttgcagaaaccaggccag | |
| | | tctccaagactcctgatctacaaagtttctaaacgattttctggggt | |
| | | cccagacaggttcagtggcagtggatcagggacagatttcacactca | |
| | | agatcagcagagtggaggctgacgatctgggaatttattactgcttt | |
| | | caaggttcacttgttccttgggcgttcggtggaggcaccaagctgga | |
| | | aatcaaa | |
| Ab198 | IPN001 VL | DVLMTQTPLSLAVNLGDQASLSCRSSQTILHSNGNTYLEWYLQKPGQ | 1937 |
| | | SPRLLIYKVSKRFSGVPDRFSGSGSGTDFTLKISRVEADDIGIYYCF | |
| | | QGSLVPWAFGGGTKLEIK | |
| Ab199 | IPN002 VL | DVXMTQSPLSLPVTLGQPASISC | 1938 |
| Ab200 | IPN002 VL- | gatgttttgatgacccaaactccactctccctgcctgtcagtcttgg | 1939 |
| | 336 | agatcaagcctccatctcttgcaaatctagtcagagcattgtacata | |
| | | gtaatggaaacacctatttagaatggtacctgcagaaaccaggccag | |
| | | tctccaaagctcctggtctacaaagtttccaatcgattttctggggt | |
| | | cccagacaggttcagtggcagtggatcagggacagatttcacactca | |
| | | agatcagcagagtggaggctgaggatctgggaacttattactgcttt | |
| | | caaggttcacttgttccttgggcgttcggtggaggcaccaagctgga | |
| | | aatcaaa | |
| Ab201 | IPN002 VL- | atgaagttgcctgttaggctgttggtgctgatgttctggattcctgc | 1940 |
| | 714 | ttccagcagtgatgttttgatgacccaaactccactctccctgcctg | |
| | | tcagtcttggagatcaagcctccatctcttgcaaatctagtcagagc | |
| | | attgtacatagtaatggaaacacctatttagaatggtacctgcagaa | |
| | | accaggccagtctccaaagctcctggtctacaaagtttccaatcgat | |
| | | tttctggggtcccagacaggttcagtggcagtggatcagggacagat | |
| | | ttcacactcaagatcagcagagtggaggctgaggatctgggaactta | |
| | | ttactgctttcaaggttcacttgttccttgggcgttcggtggaggca | |
| | | ccaagctggaaatcaaacgggctgatgctgcaccaactgtatccatc | |
| | | ttcccaccatccagtgagcagttaacatctggaggtgcctcagtcgt | |
| | | gtgcttcttgaacaacttctaccccaaagacatcaatgtcaagtgga | |
| | | agattgatggcagtgaacgacaaaatggcgtcctgaacagttggact | |
| | | gatcaggacagcaaagacagcacctacagcatgagcagcaccctcac | |
| | | gttgaccaaggacgagtatgaacgacataacagctatacctgtgagg | |
| | | ccactcacaagacatcaacttcacccattgtcaagagcttcaacagg | |
| | | aatgagtgt | |
| Ab202 | IPN002 VL- | DVLMTQTPLSLPVSLGDQASISCKSSQSIVHSNGNTYLEWYLQKPGQ | 1941 |
| | 112 | SPKLLVYKVSNRFSGVPDRFSGSGSGTDFTLKISRVEAEDLGTYYCF | |
| | | QGSLVPWAFGGGTKLEIK | |
| Ab203 | IPN002 VL | gatgttttgatgacccaaagcccactctccctgcctgtcacccttgg | 1942 |
| | variant 1 | acagcccgcctccatctcttgcaaatctagtcagagcattgtacata | |
| | | gtaatggaaacacctatttagaatggtacctgcagaaaccaggccag | |
| | | tctccacagctcctggtctacaaagtttccaatcgattttctggggt | |
| | | cccagacagattcagtggcagtggatcagggacagatttcacactca | |
| | | agatcagcagagtggaggctgaggatgtgggaacttattactgcttt | |
| | | caaggctcacttgttccttgggcgttcggtggaggcaccaaggtgga | |
| | | aatcaaa | |
| Ab204 | IPN002 VL | DVLMTQSPLSLPVTLGQPASISCKSSQSIVHSNGNTYLEWYLQKPGQ | 1943 |
| | variant 1 | SPQLLVYKVSNRFSGVPDRFSGSGSGTDFTLKISRVEAEDVGTYYCF | |
| | | QGSLVPWAFGGGTKVEIK | |
| Ab205 | IPN002 VL | gatgttgtgatgacccaaagcccactctccctgcctgtcacccttgg | 1944 |
| | variant 2 | acagcccgcctccatctcttgcaaatctagtcagagcattgtacata | |
| | | gtaatggaaacacctatttagaatggtacctgcagaaaccaggccag | |
| | | tctccacagctcctggtctacaaagtttccaatcgattttctggggt | |
| | | cccagacagattcagtggcagtggatcagggacagatttcacactca | |
| | | agatcagcagagtggaggctgaggatgtgggaacttattactgcttt | |
| | | caaggctcacttgttccttgggcgttcggtggaggcaccaaggtgga | |
| | | aatcaaa | |
| Ab206 | IPN002 VL | DVVMTQSPLSLPVTLGQPASISCKSSQSIVHSNGNTYLEWYLQKPGQ | 1945 |
| | variant 2 | SPQLLVYKVSNRFSGVPDRFSGSGSGTDFTLKISRVEAEDVGTYYCF | |
| | | QGSLVPWAFGGGTKVEIK | |
| Ab207 | IPN002 VL | gatgttgtgatgacccaaagcccactctccctgcctgtcacccttgg | 1946 |
| | variant 3 | acagcccgcctccatctcttgcaaatctagtcagagcattgtacata | |
| | | gtaatggaaacacctatttagaatggtacctgcagaaaccaggccag | |
| | | tctccacagctcctggtctacaaagtttccaatcgattttctggggt | |
| | | cccagacagattcagtggcagtggatcagggacagatttcacactca | |
| | | agatcagcagagtggaggctgaggatgtgggagtgtattactgcttt | |
| | | caaggctcacttgttccttgggcgttcggtggaggcaccaaggtgga | |
| | | aatcaaa | |
| Ab208 | IPN002 VL | DVVMTQSPLSLPVTLGQPASISCKSSQSIVHSNGNTYLEWYLQKPGQ | 1947 |
| | variant 3 | SPQLLVYKVSNRFSGVPDRFSGSGSGTDFTLKISRVEAEDVGVYYCF | |
| | | QGSLVPWAFGGGTKVEIK | |
| Ab209 | IPN002 VL | gatgttgtgatgacccaaagcccactctccctgcctgtcacccttgg | 1948 |
| | variant 4 | acagcccgcctccatctcttgcaaatctagtcagagcattgtacata | |
| | | gtaatggaaacacctatttagaatggtacctgcagaaaccaggccag | |
| | | tctccacagctcctgatctacaaagtttccaatcgattttctggggt | |
| | | cccagacagattcagtggcagtggatcagggacagatttcacactca | |
| | | agatcagcagagtggaggctgaggatgtgggagtgtattactgcttt | |
| | | caaggctcacttgttccttgggcgttcggtggaggcaccaaggtgga | |
| | | aatcaaa | |
| Ab210 | IPN002 VL | DVVMTQSPLSLPVTLGQPASISCKSSQSIVHSNGNTYLEWYLQKPGQ | 1949 |
| | variant 4 | SPQLLIYKVSNRFSGVPDRFSGSGSGTDFTLKISRVEAEDVGVYYCF | |
| | | QGSLVPWAFGGGTKVEIK | |
| Ab211 | MC1 VL- | atgaagttgcctgttaggctgttggtgctgatgttctggattcctgc | 1950 |
| | 714 | ttccagcagtgatgttgtgatgacccaaactccactctccctgcctg | |
| | | tcagtcttggagatcaagcctccatctcttgcagatctagtcagagc | |
| | | cttgtacacagtaatggaaacacctatttacattggtacctgcagaa | |
| | | gccaggccagtctccaaagctcctgatctacaaagtttccaaccgat | |
| | | tttctggggtcccagacaggttcagtggcagtggatcagggacagat | |
| | | ttcacactcaagatcagcagagtggaggctgaggatctgggagttta | |
| | | tttctgctctcaaagtacacatgttccgctcacgttcggtgctggga | |
| | | ccaagctggagctgaaacgggctgatgctgcaccaactgtatccatc | |
| | | ttcccaccatccagtgagcagttaacatctggaggtgcctcagtcgt | |
| | | gtgcttcttgaacaacttctaccccaaagacatcaatgtcaagtgga | |
| | | agattgatggcagtgaacgacaaaatggcgtcctgaacagttggact | |
| | | gatcaggacagcaaagacagcacctacagcatgagcagcaccctcac | |
| | | gttgaccaaggacgagtatgaacgacataacagctatacctgtgagg | |
| | | ccactcacaagacatcaacttcacccattgtcaagagcttcaacagg | |
| | | aatgagtgt | |
| Ab212 | MC1 VL- | atgaagttgcctgttaggctgttggtgctgatgttctggattcctgc | 1951 |
| | 717 | ttccagcagtgatgttgtgatgacccaaactccactctccctgcctg | |
| | | tcagtcttggagatcaagcctccatctcttgcagatctagtcagagc | |
| | | cttgtacacagtaatggaaacacctatttacattggtacctgcagaa | |
| | | gccaggccagtctccaaagctcctgatctacaaagtttccaaccgat | |
| | | tttctggggtcccagacaggttcagtggcagtggatcagggacagat | |
| | | ttcacactcaagatcagcagagtggaggctgaggatctgggagttta | |
| | | tttctgctctcaaagtacacatgttccgctcacgttcggtgctggga | |
| | | ccaagctggagctgaaacgggctgatgctgcaccaactgtatccatc | |
| | | ttcccaccatccagtgagcagttaacatctggaggtgcctcagtcgt | |
| | | gtgcttcttgaacaacttctaccccaaagacatcaatgtcaagtgga | |
| | | agattgatggcagtgaacgacaaaatggcgtcctgaacagttggact | |
| | | gatcaggacagcaaagacagcacctacagcatgagcagcaccctcac | |
| | | gttgaccaaggacgagtatgaacgacataacagctatacctgtgagg | |
| | | ccactcacaagacatcaacttcacccattgtcaagagcttcaacagg | |
| | | aatgagtgttag | |
| Ab213 | MC1 VL- | gaaattgtgttgacgcagtctccaggcaccctgtctttgtctccagg | 1952 |
| | 657 | ggaaagagccaccctctcctgcagatctagtcagagccttgtacaca | |
| | | gtaatcagaacacctatttacattggtaccagcagaaacctggccag | |
| | | gctcccaggctcctcatctataaagttgacaaccgattttctggcat | |
| | | cccagacaggttcagtggcagtgggtctgggacagacttcactctca | |
| | | ccatcagcagactggagcctgaagattttgcagtgtattactgttct | |
| | | caaagtacactggttccgctcacgttcggcggagggaccaaggtgga | |
| | | gatcaaacggaccgtggctgcaccatctgtcttcatcttcccgccat | |
| | | ctgatgagcagttgaaatctggaactgcctctgttgtgtgcctgctg | |
| | | aataacttctatcccagagaggccaaagtacagtggaaggtggataa | |
| | | cgccctccaatcgggtaactcccaggagagtgtcacagagcaggaca | |
| | | gcaaggacagcacctacagcctcagcagcaccctgacgctgagcaaa | |
| | | gcagactacgagaaacacaaagtctacgcctgcgaagtcacccatca | |
| | | gggcctgagctcgcccgtcacaaagagcttcaacaggggagagtgc | |
| Ab214 | MC1 VL- | MKLPVRLLVLMFWIPASSSDVVMTQTPLSLPVSLGDQASISCRSSQS | 1953 |
| | 238 | LVHSNGNTYLHWYLQKPGQSPKLLIYKVSNRFSGVPDRFSGSGSGTD | |
| | | FTLKISRVEAEDLGVYFCSQSTHVPLTFGAGTKLELKRADAAPTVSI | |
| | | FPPSSEQLTSGGASVVCFLNNFYPKDINVKWKIDGSERQNGVLNSWT | |
| | | DQDSKDSTYSMSSTLTLTKDEYERHNSYTCEATHKTSTSPIVKSENR | |
| | | NEC | |
| Ab215 | MC1 VL- | EIVLTQSPGILSLSPGERATLSCRSSQSLVHSNQNTYLHWYQQKPGQ | 1954 |
| | 219 | APRLLIYKVDNRFSGIPDRFSGSGSGIDFTLTISRLEPEDFAVYYCS | |
| | | QSTLVPLTFGGGTKVEIKRTVAAPSVFIFPPSDEQLKSGTASVVCLL | |
| | | NNFYPREAKVQWKVDNALQSGNSQESVTEQDSKDSTYSLSSTLTLSK | |
| | | ADYEKHKVYACEVTHQGLSSPVIKSENRGEC | |
| Ab216 | MC1 VL- | EIVLTQSPGTLSLSPGERATLSCRSSQSLVHSNQNTYLHWYQQKPGQ | 1955 |
| | 112 | APRLLIYKVDNRFSGIPDRFSGSGSGTDFTLTISRLEPEDFAVYYCS | |
| | | QSTLVPLTFGGGTKVEIK | |
| Ab217 | PHF1 VL- | gatgttgtgatgacccagactccactcactttgtcggttaccattgg | 1956 |
| | 339v1 | acaaccagcctccatctcttgcaagtcaagtcagagcctcttagata | |
| | | gtgatggaaagacatatttgaattggttgttacagaggccaggccag | |
| | | tctccaaagcgcctaatctatctggtgtctaaactggactctggagt | |
| | | ccccgacagattcactggcagtggatcagggacagatttcacactga | |
| | | aaattagcagagtggaggctgaggatttgggagtttattattgctgg | |
| | | caaggtacacattttcctcggacgttcggtggaggcaccaagctgga | |
| | | aatcaaacgg | |
| Ab218 | PHF1 VL- | gatgttgtgatgacccagactccactcactttgtcggttaccattgg | 1957 |
| | 339v2 | acaaccagcctccatctcttgcaagtcaagtcagagcctcttagata | |
| | | gtgatggaaagacatatttgaattggttgttacagaggccaggccag | |
| | | tctccaaagcgcctaatctatctggtgtctaaactggactctggagt | |
| | | ccctgacagattcactggcagtggatcagggacagatttcacactga | |
| | | aaattagcagagtggaggctgaggatttgggagtttattattgctgg | |
| | | caaggtacacattttcctcggacgttcggtggaggcaccaagctgga | |
| | | aatcaaacgg | |
| Ab219 | PHF1 VL- | atgagtcctgcccagttcctgtttctgttagtgctctggattcggga | 1958 |
| | 396 | aaccaacggtgatgttgtgatgacccagactccactcactttgtcgg | |
| | | ttaccattggacaaccagcctccatctcttgcaagtcaagtcagagc | |
| | | ctcttagatagtgatggaaagacatatttgaattggttgttacagag | |
| | | gccaggccagtctccaaagcgcctaatctatctggtgtctaaactgg | |
| | | actctggagtccccgacagattcactggcagtggatcagggacagat | |
| | | ttcacactgaaaattagcagagtggaggctgaggatttgggagttta | |
| | | ttattgctggcaaggtacacattttcctcggacgttcggtggaggca | |
| | | ccaagctggaaatcaaacgg | |
| Ab220 | PHF1 VL- | gatgttgtgatgacccagactccactcactttgtcggttaccattgg | 1959 |
| | 657 | acaaccagcctccatctcttgcaagtcaagtcagagcctcttagata | |
| | | gtgatggaaagacatatttgaattggttgttacagaggccaggccag | |
| | | tctccaaagcgcctaatctatctggtgtctaaactggactctggagt | |
| | | ccctgacagattcactggcagtggatcagggacagatttcacactga | |
| | | aaattagcagagtggaggctgaggatttgggagtttattattgctgg | |
| | | caaggtacacattttcctcggacgttcggtggaggcaccaagctgga | |
| | | aatcaaacgggctgatgctgcaccaactgtatccatcttcccaccat | |
| | | ccagtgagcagttaacatctggaggtgcctcagtcgtgtgcttcttg | |
| | | aacaacttctaccccaaagacatcaatgtcaagtggaagattgatgg | |
| | | cagtgaacgacaaaatggcgtcctgaacagttggactgatcaggaca | |
| | | gcaaagacagcacctacagcatgagcagcaccctcacgttgaccaag | |
| | | gacgagtatgaacgacataacagctatacctgtgaggccactcacaa | |
| | | gacatcaacttcacccattgtcaagagcttcaacaggaatgagtgt | |
| Ab221 | PHF1 VL- | gatgttgtgatgacccagactccactcactttgtcggttaccattgg | 1960 |
| | 660 | acaaccagcctccatctcttgcaagtcaagtcagagcctcttagata | |
| | | gtgatggaaagacatatttgaattggttgttacagaggccaggccag | |
| | | tctccaaagcgcctaatctatctggtgtctaaactggactctggagt | |
| | | ccctgacagattcactggcagtggatcagggacagatttcacactga | |
| | | aaattagcagagtggaggctgaggatttgggagtttattattgctgg | |
| | | caaggtacacattttcctcggacgttcggtggaggcaccaagctgga | |
| | | aatcaaacgggctgatgctgcaccaactgtatccatcttcccaccat | |
| | | ccagtgagcagttaacatctggaggtgcctcagtcgtgtgcttcttg | |
| | | aacaacttctaccccaaagacatcaatgtcaagtggaagattgatgg | |
| | | cagtgaacgacaaaatggcgtcctgaacagttggactgatcaggaca | |
| | | gcaaagacagcacctacagcatgagcagcaccctcacgttgaccaag | |
| | | gacgagtatgaacgacataacagctatacctgtgaggccactcacaa | |
| | | gacatcaacttcacccattgtcaagagcttcaacaggaatgagtgtt | |
| | | ag | |
| Ab222 | PHF1 VL- | gatgttgtgatgacccagactccactcactttgtcggttaccattgg | 1961 |
| | 678 | acaaccagcctccatctcttgcaagtcaagtcagagcctcttagata | |
| | | gtgatggaaagacatatttgaattggttgttacagaggccaggccag | |
| | | tctccaaagcgcctaatctatctggtgtctaaactggactctggagt | |
| | | ccctgacagattcactggcagtggatcagggacagatttcacactga | |
| | | aaattagcagagtggaggctgaggatttgggagtttattattgctgg | |
| | | caaggtacacattttcctcggacgttcggtggaggcaccaagctgga | |
| | | aatcaaacgggctgatgctgcaccaactgtatccatcttcccaccat | |
| | | ccagtgagcagttaacatctggaggtgcctcagtcgtgtgcttcttg | |
| | | aacaacttctaccccaaagacatcaatgtcaagtggaagattgatgg | |
| | | cagtgaacgacaaaatggcgtcctgaacagttggactgatcaggaca | |
| | | gcaaagacagcacctacagcatgagcagcaccctcacgttgaccaag | |
| | | gacgagtatgaacgacataacagctatacctgtgaggccactcacaa | |
| | | gacatcaacttcacccattgtcaagagcttcaacaggaatgagtgta | |
| | | gcgagaaggacgagctgtag | |
| Ab223 | PHF1 VL- | atgagtcctgcccagttcctgtttctgttagtgctctggattcggga | 1962 |
| | 714 | aaccaacggtgatgttgtgatgacccagactccactcactttgtcgg | |
| | | ttaccattggacaaccagcctccatctcttgcaagtcaagtcagagc | |
| | | ctcttagatagtgatggaaagacatatttgaattggttgttacagag | |
| | | gccaggccagtctccaaagcgcctaatctatctggtgtctaaactgg | |
| | | actctggagtccctgacagattcactggcagtggatcagggacagat | |
| | | ttcacactgaaaattagcagagtggaggctgaggatttgggagttta | |
| | | ttattgctggcaaggtacacattttcctcggacgttcggtggaggca | |
| | | ccaagctggaaatcaaacgggctgatgctgcaccaactgtatccatc | |
| | | ttcccaccatccagtgagcagttaacatctggaggtgcctcagtcgt | |
| | | gtgcttcttgaacaacttctaccccaaagacatcaatgtcaagtgga | |
| | | agattgatggcagtgaacgacaaaatggcgtcctgaacagttggact | |
| | | gatcaggacagcaaagacagcacctacagcatgagcagcaccctcac | |
| | | gttgaccaaggacgagtatgaacgacataacagctatacctgtgagg | |
| | | ccactcacaagacatcaacttcacccattgtcaagagcttcaacagg | |
| | | aatgagtgt | |
| Ab224 | PHF1 VL- | atgatgagtcctgcccagttcctgtttctgttagtgctctggattcg | 1963 |
| | 720 | ggaaaccaacggtgatgttgtgatgacccagactccactcactttgt | |
| | | cggttaccattggacaaccagcctccatctcttgcaagtcaagtcag | |
| | | agcctcttagatagtgatggaaagacatatttgaattggttgttaca | |
| | | gaggccaggccagtctccaaagcgcctaatctatctggtgtctaaac | |
| | | tggactctggagtccctgacagattcactggcagtggatcagggaca | |
| | | gatttcacactgaaaattagcagagtggaggctgaggatttgggagt | |
| | | ttattattgctggcaaggtacacattttcctcggacgttcggtggag | |
| | | gcaccaagctggaaatcaaacgggctgatgctgcaccaactgtatcc | |
| | | atcttcccaccatccagtgagcagttaacatctggaggtgcctcagt | |
| | | cgtgtgcttcttgaacaacttctaccccaaagacatcaatgtcaagt | |
| | | ggaagattgatggcagtgaacgacaaaatggcgtcctgaacagttgg | |
| | | actgatcaggacagcaaagacagcacctacagcatgagcagcaccct | |
| | | cacgttgaccaaggacgagtatgaacgacataacagctatacctgtg | |
| | | aggccactcacaagacatcaacttcacccattgtcaagagcttcaac | |
| | | aggaatgagtgttag | |
| Ab225 | PHF1 VL- | MMSPAQFLFLLVLWIRETNGDVVMTQTPLTLSVTIGQPASISCKSSQ | 1964 |
| | 239 | SLLDSDGKTYLNWLLQRPGQSPKRLIYLVSKLDSGVPDRFTGSGSGT | |
| | | DFTLKISRVEAEDLGVYYCWQGTHFPRTFGGGTKLEIKRADAAPTVS | |
| | | IFPPSSEQLTSGGASVVCFLNNFYPKDINVKWKIDGSERQNGVLNSW | |
| | | TDQDSKDSTYSMSSTLTLTKDEYERHNSYTCEATHKISTSPIVKSEN | |
| | | RNEC | |
| Ab226 | PHF1 VL + | gccgccaccatgggatggagctggatctttctctttctcctgtcagg | 1965 |
| | (732) | aactacaggtgtcctctctgctgccgatgttgtgatgacccagactc | |
| | | cactcactttgtcggttaccattggacaaccagcctccatctcttgc | |
| | | aagtcaagtcagagcctcttagatagtgatggaaagacatatttgaa | |
| | | ttggttgttacagaggccaggccagtctccaaagcgcctaatctatc | |
| | | tggtgtctaaactggactctggagtccctgacagattcactggcagt | |
| | | ggatcagggacagatttcacactgaaaattagcagagtggaggctga | |
| | | ggatttgggagtttattattgctggcaaggtacacattttcctcgga | |
| | | cgttcggtggaggcaccaagctggaaatcaaacgggctgatgctgca | |
| | | ccaactgtatccatcttcccaccatccagtgagcagttaacatctgg | |
| | | aggtgcctcagtcgtgtgcttcttgaacaacttctaccccaaagaca | |
| | | tcaatgtcaagtggaagattgatggcagtgaacgacaaaatggcgtc | |
| | | ctgaacagttggactgatcaggacagcaaagacagcacctacagcat | |
| | | gagcagcaccctcacgttgaccaaggacgagtatgaacgacataaca | |
| | | gctatacctgtgaggccactcacaagacatcaacttcacccattgtc | |
| | | aagagcttcaacaggaatgagtgttag | |
| Ab227 | PHF1 VL + | atgatgagtcctgcccagttcctgtttctgttagtgctctggattcg | 1966 |
| | (747) | ggaaaccaacggtgatgttgtgatgacccagactccactcactttgt | |
| | | cggttaccattggacaaccagcctccatctcttgcaagtcaagtcag | |
| | | agcctcttagatagtgatggaaagacatatttgaattggttgttaca | |
| | | gaggccaggccagtctccaaagcgcctaatctatctggtgtctaaac | |
| | | tggactctggagtccctgacagattcactggcagtggatcagggaca | |
| | | gatttcacactgaaaattagcagagtggaggctgaggatttgggagt | |
| | | ttattattgctggcaaggtacacattttcctcggacgttcggtggag | |
| | | gcaccaagctggaaatcaaacgggctgatgctgcaccaactgtatcc | |
| | | atcttcccaccatccagtgagcagttaacatctggaggtgcctcagt | |
| | | cgtgtgcttcttgaacaacttctaccccaaagacatcaatgtcaagt | |
| | | ggaagattgatggcagtgaacgacaaaatggcgtcctgaacagttgg | |
| | | actgatcaggacagcaaagacagcacctacagcatgagcagcaccct | |
| | | cacgttgaccaaggacgagtatgaacgacataacagctatacctgtg | |
| | | aggccactcacaagacatcaacttcacccattgtcaagagcttcaac | |
| | | aggaatgagtgttacccatacgatgttccagattacgcttag | |
| Ab228 | PT1 VL- | gatattgtgatgactcaggctgcaccctctgtacctgtcactcctgg | 1967 |
| | 336 | agagtcagtttccatctcctgcaggtctagtgagagtctcctgcata | |
| | | gtaatggcaacacttacttgtattggttcctgcagaggccaggccag | |
| | | tctcctcagctcctgatatatcggatgtccaaccttgcctcaggagt | |
| | | cccagacaggttcagtggcagtgggtcaggaactgctttcacactga | |
| | | gaatcagtagagtggaggctgaggatgtgggtgtttattactgtatg | |
| | | caatatctagaatatccgctcacgttcggtgctgggaccaagctgga | |
| | | gctgaaa | |
| Ab229 | PT1 VL- | DIVMTQAAPSVPVTPGESVSISCRSSESLLHSNGNTYLYWFLQRPGQ | 1968 |
| | 112 | SPQLLIYRMSNLASGVPDRFSGSGSGTAFTLRISRVEAEDVGVYYCM | |
| | | QYLEYPLTFGAGTKLELK | |
| Ab230 | PT3 VL- | gacatcaagatgacccagtctccatcttccatgtatgcatctctagg | 1969 |
| | 321 | agagagagtcactatcacttgcaaggcgagtcaggacattaataggt | |
| | | atttaaactggttccagcagaaaccagggaaatctcctaagaccctg | |
| | | atctatcgtgcaaacagattgctagatggggtcccatcaaggttcag | |
| | | tggcagtggatctgggcaagattactctctcaccatcagcagcctgg | |
| | | attatgaagatatgggaatttattattgtctacagtatgatgagttt | |
| | | ccgctcacgttcggtgatgggaccaagctggagctgaaa | |
| Ab231 | PT3 VL- | DIKMTQSPSSMYASLGERVTITCKASQDINRYLNWFQQKPGKSPKTL | 1970 |
| | 107 | IYRANRLLDGVPSRFSGSGSGQDYSLTISSLDYEDMGIYYCLQYDEF | |
| | | PLTFGDGTKLELK | |
| Ab232 | PT3 VL | gatattaaaatgacccagagcccgagcagcatgtatgcgagcctggg | 1971 |
| | Bioinformatics | cgaacgcgtgaccattacctgcaaagcgagccaggatattaaccgct | |
| | | atctgaactggtttcagcagaaaccgggcaaaagcccgaaaaccctg | |
| | | atttatcgcgcgaaccgcctgctggatggcgtgccgagccgctttag | |
| | | cggcagcggcagcggccaggattatagcctgaccattagcagcctgg | |
| | | attatgaagatatgggcatttattattgcctgcagtatgatgaattt | |
| | | ccgctgacctttggcgatggcaccaaactggaactgaaa | |
| Ab233 | PT3 VL | gatatcaagatgacccagagccccagctccatgtatgccagcctggg | 1972 |
| | Bioinformatics | ggagcgggtgaccatcacctgcaaggcctcccaggacatcaaccgct | |
| | 2 | acctgaactggttccagcagaagccaggcaagtcccccaagaccctc | |
| | | atctatcgggccaaccggctgctggatggagtgccctcccgcttctc | |
| | | cggcagtggcagcggccaggattacagcctcaccatttcttccctgg | |
| | | actatgaggacatgggcatctactactgcctgcagtatgatgagttc | |
| | | cccctgacctttggagatggcaccaagctggagctgaag | |
| Ab234 | PT3 VL | gacatcaagatgacccagagccccagcagcatgtacgccagcctggg | 1973 |
| | EMBOSS | cgagagggtgaccatcacctgcaaggccagccaggacatcaacaggt | |
| | | acctgaactggttccagcagaagcccggcaagagccccaagaccctg | |
| | | atctacagggccaacaggctgctggacggcgtgcccagcaggttcag | |
| | | cggcagcggcagcggccaggactacagcctgaccatcagcagcctgg | |
| | | actacgaggacatgggcatctactactgcctgcagtacgacgagttc | |
| | | cccctgaccttcggcgacggcaccaagctggagctgaag | |
| Ab235 | PT3 VL | gacatcaagatgacccagagcccttcctccatgtatgcctccctggg | 1974 |
| | Geneinfinity | ggagcgggtgaccatcacctgcaaggccagccaggatatcaaccgct | |
| | | acctgaactggttccagcagaagccaggcaagagccccaagaccctc | |
| | | atctatcgggccaaccggctgctggatggagtgcccagccgcttctc | |
| | | cggcagcggcagtggccaggactacagcctcaccatttccagcctgg | |
| | | actatgaggacatgggcatctactactgcctgcagtatgatgagttc | |
| | | cccctgacctttggagatggcaccaagctggagctgaag | |
| Ab236 | PT3 VL | gacatcaagatgacccagtcccccagctccatgtacgcctctctggg | 1975 |
| | Genescript | agagcgggtgaccatcacatgcaaggcttctcaggatatcaaccgct | |
| | | acctgaactggttccagcagaagcccggaaagagccctaagaccctg | |
| | | atctacagggccaacagactgctggacggcgtgccaagcaggttttc | |
| | | cggctctggaagcggccaggattactccctgacaatctctagcctgg | |
| | | actacgaggatatgggcatctactactgtctgcagtacgacgagttc | |
| | | cccctgacctttggagatggcacaaagctggagctgaag | |
| Ab237 | PT3 VL | gacatcaagatgacccagagcccttcctccatgtatgcctccctggg | 1976 |
| | IDT | ggagcgggtgaccatcacctgcaaggccagccaggatatcaaccgct | |
| | | acctgaactggttccagcagaagcccggcaagagccccaagaccctc | |
| | | atctatcgggccaaccggctgctggatggagtgcccagccgcttctc | |
| | | cggcagcggcagtggccaggactacagcctgaccatttcttctctgg | |
| | | actatgaggacatgggcatctactactgcctgcagtatgatgagttc | |
| | | cccctgacatttggagatggcaccaagctggagctgaag | |
| Ab238 | PT3 VL | cgggctgatgctgcaccaactgtatccatcttcccaccatccagtga | 1977 |
| | NUS | gcagttaacatctggaggtgcctcagtcgtgtgcttcttgaacaact | |
| | | tctaccccaaagacatcaatgtcaagtggaagattgatggcagtgaa | |
| | | cgacaaaatgacatcaagatgacccagagcccttcctccatgtatgc | |
| | | ctccctgggggagcgggtgacaatcacctgcaaggccagccaggata | |
| | | tcaaccgctacctgaactggttccagcagaagccaggcaagagcccc | |
| | | aagaccctcatctatagagccaaccggctgctggacggc | |
| Ab239 | PT3 VL | gacatcaagatgacccagagcccttcttccatgtatgcttctttagg | 1978 |
| | NUS2 | agaaagagtgacaatcacctgcaaggccagccaggatatcaaccgct | |
| | | acctgaactggttccagcagaagccaggaaaatctcccaagaccctg | |
| | | atttatagagccaacagactgctggacggcgtgcccagccgcttcag | |
| | | tggatctggaagtggccaggactacagcctgaccatctcttctctgg | |
| | | attatgaagacatgggcatctactactgcctgcagtatgatgaattt | |
| | | cctttaacatttggagatggcaccaagctggagctgaag | |
| Ab240 | PT3 VL | gatatcaagatgacccagagcccttcctccatgtatgcctccctggg | 1979 |
| | NUS3 | ggagcgggtgaccatcacctgcaaggccagccaggacatcaaccgct | |
| | | acctgaactggttccagcagaagcccggcaagagccccaagaccctc | |
| | | atctatcgggccaaccggctgctggatggagtgcccagccgcttctc | |
| | | cggcagcggcagtggccaggattacagcctcaccatttccagcctgg | |
| | | actatgaggacatgggcatctactactgcctgcagtatgatgagttc | |
| | | cccctgacctttggagatggcaccaagctggagctgaag | |
| Ab241 | PT3 VL | gacatcaagatgacccagagccctagcagcatgtacgccagcctggg | 1980 |
| | Snapgene | cgagagagtgaccatcacctgcaaggccagccaggacatcaacagat | |
| | | acctgaactggttccagcagaagcctggcaagagccctaagaccctg | |
| | | atctacagagccaacagactgctggacggcgtgcctagcagattcag | |
| | | cggcagcggcagcggccaggactacagcctgaccatcagcagcctgg | |
| | | actacgaggacatgggcatctactactgcctgcagtacgacgagttc | |
| | | cctctgaccttcggcgacggcaccaagctggagctgaag | |
| Ab242 | VL | DVLMTQSPLSLPVTLGQPASISC | 1981 |
| Ab243 | AT8 Fab1 | RSGYSSPGXPGXPGSRSR | 1982 |
| Ab244 | AT8 Fab2 | RSGYSSPGXPGXPGXRSR | 1983 |
| Ab245 | MC-1 | gaggagccaccatggaatggacctgggtctttctcttcctcctgtca | 1984 |
| | Furin 2A- | gtaactgcaggtgtccactcccaggttcagctgcagcagtctggagc | |
| | 2168 | tgagctgatgaagcctggggcctcagtgaagatatcctgcaaggcta | |
| | | ctggctacacattcagtaactactggatagagtgggtaaagcagagg | |
| | | cctggacatggccttgagtggattggagagattttacctggaagtga | |
| | | tagtattaagtacaatgagaatttcaagggcaaggccacattcactg | |
| | | cagatacatcctccaacacagcctacatgcaactcagcagcctgaca | |
| | | tctgaggactctgccgtctattactgtgcaagaagggggaactaccc | |
| | | agactactggggccaaggcaccactctcacagtctcctcagccaaaa | |
| | | cgacacccccatctgtctatccactggcccctggatctgctgcccaa | |
| | | actaactccatggtgaccctgggatgcctggtcaagggctatttccc | |
| | | tgagccagtgacagtgacctggaactctggatccctgtccagcggtg | |
| | | tgcacaccttcccagctgtcctgcagtctgacctctacactctgagc | |
| | | agctcagtgactgtcccctccagcacctggcccagcgagaccgtcac | |
| | | ctgcaacgttgcccacccggccagcagcaccaaggtggacaagaaaa | |
| | | ttgtgcccagggattgtggttgtaagccttgcatatgtacagtccca | |
| | | gaagtatcatctgtcttcatcttccccccaaagcccaaggatgtgct | |
| | | caccattactctgactcctaaggtcacgtgtgttgtggtagacatca | |
| | | gcaaggatgatcccgaggtccagttcagctggtttgtagatgatgtg | |
| | | gaggtgcacacagctcagacgcaaccccgggaggagcagttcaacag | |
| | | cactttccgctcagtcagtgaacttcccatcatgcaccaggactggc | |
| | | tcaatggcaaggagttcaaatgcagggtcaacagtgcagctttccct | |
| | | gcccccatcgagaaaaccatctccaaaaccaaaggcagaccgaaggc | |
| | | tccacaggtgtacaccattccacctcccaaggagcagatggccaagg | |
| | | ataaagtcagtctgacctgcatgataacagacttcttccctgaagac | |
| | | attactgtggagtggcagtggaatgggcagccagcggagaactacaa | |
| | | gaacactcagcccatcatggacacagatggctcttacttcgtctaca | |
| | | gcaagctcaatgtgcagaagagcaactgggaggcaggaaatactttc | |
| | | acctgctctgtgttacatgagggcctgcacaaccaccatactgagaa | |
| | | gagcctctcccactctcctggtagaaagaggcgagagggcagaggaa | |
| | | gtcttctaacatgcggtgacgtggaggagaatcccggccctatgaag | |
| | | ttgcctgttaggctgttggtgctgatgttctggattcctgcttccag | |
| | | cagtgatgttgtgatgacccaaactccactctccctgcctgtcagtc | |
| | | ttggagatcaagcctccatctcttgcagatctagtcagagccttgta | |
| | | cacagtaatggaaacacctatttacattggtacctgcagaagccagg | |
| | | ccagtctccaaagctcctgatctacaaagtttccaaccgattttctg | |
| | | gggtcccagacaggttcagtggcagtggatcagggacagatttcaca | |
| | | ctcaagatcagcagagtggaggctgaggatctgggagtttatttctg | |
| | | ctctcaaagtacacatgttccgctcacgttcggtgctgggaccaagc | |
| | | tggagctgaaacgggctgatgctgcaccaactgtatccatcttccca | |
| | | ccatccagtgagcagttaacatctggaggtgcctcagtcgtgtgctt | |
| | | cttgaacaacttctaccccaaagacatcaatgtcaagtggaagattg | |
| | | atggcagtgaacgacaaaatggcgtcctgaacagttggactgatcag | |
| | | gacagcaaagacagcacctacagcatgagcagcaccctcacgttgac | |
| | | caaggacgagtatgaacgacataacagctatacctgtgaggccactc | |
| | | acaagacatcaacttcacccattgtcaagagcttcaacaggaatgag | |
| | | tgttag | |
| Ab246 | MC-1 | MEWTWVFLFLLSVTAGVHSQVQLQQSGAELMKPGASVKISCKATGYT | 1985 |
| | Furin 2A- | FSNYWIEWVKQRPGHGLEWIGEILPGSDSIKYNENFKGKATFTADTS | |
| | 718 | SNTAYMQLSSLTSEDSAVYYCARRGNYPDYWGQGTTLTVSSAKTTPP | |
| | | SVYPLAPGSAAQTNSMVTLGCLVKGYFPEPVTVTWNSGSLSSGVHIF | |
| | | PAVLQSDLYTLSSSVTVPSSTWPSETVTCNVAHPASSTKVDKKIVPR | |
| | | DCGCKPCICTVPEVSSVFIFPPKPKDVLTITLTPKVTCVVVDISKDD | |
| | | PEVQFSWFVDDVEVHTAQTQPREEQFNSTFRSVSELPIMHQDWLNGK | |
| | | EFKCRVNSAAFPAPIEKTISKTKGRPKAPQVYTIPPPKEQMAKDKVS | |
| | | LTCMITDFFPEDITVEWQWNGQPAENYKNTQPIMDTDGSYFVYSKLN | |
| | | VQKSNWEAGNTFTCSVLHEGLHNHHTEKSLSHSPGRKRREGRGSLLT | |
| | | CGDVEENPGPMKLPVRLLVLMFWIPASSSDVVMTQTPLSLPVSLGDQ | |
| | | ASISCRSSQSLVHSNGNTYLHWYLQKPGQSPKLLIYKVSNRFSGVPD | |
| | | RFSGSGSGTDFTLKISRVEAEDLGVYFCSQSTHVPLTFGAGTKLELK | |
| | | RADAAPTVSIFPPSSEQLISGGASVVCFLNNFYPKDINVKWKIDGSE | |
| | | RQNGVLNSWTDQDSKDSTYSMSSTLTLTKDEYERHNSYTCEATHKTS | |
| | | TSPIVKSFNRNEC | |
| Ab247 | MC-1 | gaggagccaccatggagtggacctgggtgttcctgttcctgctgagc | 1986 |
| | optimized | gtgaccgccggcgtgcactcccaagtccagctgcagcagagcggcgc | |
| | seq | cgagctgatgaagcccggcgccagcgtgaagatcagctgcaaggcca | |
| | | ccggctacaccttcagcaactactggatcgagtgggtgaagcagcgg | |
| | | cccggccacggcctggagtggatcggcgagatcctgcccggcagcga | |
| | | cagcatcaagtacaacgagaacttcaagggcaaggccaccttcaccg | |
| | | ccgacaccagcagcaacaccgcctacatgcagctgagcagcctgacc | |
| | | agcgaggacagcgccgtgtactactgcgcccggcggggcaactaccc | |
| | | cgactactggggccagggcaccaccctgaccgtctccagcgccaaga | |
| | | ccacaccacccagcgtgtacccactggctccaggcagcgctgcccag | |
| | | accaacagcatggtgaccctgggctgcctggtgaagggctacttccc | |
| | | cgagcccgtgaccgtgacctggaacagcggcagcctgagcagcggcg | |
| | | tgcacaccttcccagccgtgctccaaagcgacctgtacacactgagc | |
| | | agcagcgtgaccgtgcccagcagcacctggcccagcgagaccgtgac | |
| | | ctgcaacgtggcccaccccgccagcagcaccaaggtggacaagaaga | |
| | | tcgtgccccgggactgcggctgcaagccctgcatctgcaccgtgccc | |
| | | gaagtcagcagcgtgttcatcttcccacccaagcccaaggacgtgct | |
| | | gaccatcaccctgacacccaaagtcacctgcgtggtggtggacatca | |
| | | gcaaggacgaccccgaggtgcagttcagctggttcgtggacgacgtg | |
| | | gaggtgcacaccgcccagacccagccccgggaggagcagttcaacag | |
| | | caccttccggagcgtgagcgagctgcccatcatgcaccaggactggc | |
| | | tgaacggcaaggagttcaagtgccgggtgaacagcgccgccttcccc | |
| | | gcccccatcgagaagaccatcagcaagaccaagggacggcccaaggc | |
| | | tccccaggtgtacaccatcccaccacccaaggagcagatggccaagg | |
| | | acaaagtcagcctgacctgcatgatcaccgacttcttccccgaggac | |
| | | atcaccgtggagtggcagtggaacggccagcccgccgagaactacaa | |
| | | gaacacccagcccatcatggacaccgacggcagctacttcgtgtaca | |
| | | gcaagctgaacgtgcagaagagcaactgggaggccggcaacaccttc | |
| | | acctgcagcgtgctgcacgagggcctgcacaaccatcacaccgagaa | |
| | | gagcctgagccacagcccaggacggaagcgccgcgagggcaggggca | |
| | | gcctgctgacctgcggcgacgtggaggagaacccaggccccatgaag | |
| | | ctgcccgtgcggctgctggtgctgatgttctggatccccgccagcag | |
| | | cagcgacgtggtgatgacccagaccccactgagcctgcccgtgagcc | |
| | | tgggcgaccaggccagcatcagctgccggagcagccagagcctggtg | |
| | | cacagcaacggcaacacctacctgcactggtacctgcagaagcccgg | |
| | | ccagagccccaagctgctgatctacaaagtcagcaaccggttcagcg | |
| | | gcgtgcccgaccggttcagcggcagcggcagcggcaccgacttcacc | |
| | | ctgaagatcagccgggtggaggccgaggacctgggcgtgtacttctg | |
| | | cagccagagcacccacgtgcccctgaccttcggcgccggcaccaagc | |
| | | tggagctgaagcgggccgacgccgctcccaccgtcagcatcttccca | |
| | | ccctcctccgagcagctgaccagcggaggcgccagcgtggtgtgctt | |
| | | cctgaacaacttctaccccaaggacatcaacgtgaagtggaagatcg | |
| | | acggcagcgagcggcagaacggcgtgctgaacagctggaccgaccag | |
| | | gacagcaaggacagcacctacagcatgagcagcaccctgaccctgac | |
| | | caaggacgagtacgagcggcacaacagctacacctgcgaggccaccc | |
| | | acaagaccagcaccagccccatcgtgaagagcttcaaccggaacgag | |
| | | tgctga | |
| Ab248 | PHF-1 | gaggagccaccatgggatggagctggatctttctctttctcctgtca | 1987 |
| | Furin 2A- | ggaactacaggtgtcctctctgaggtccagctgcaacagtctggacc | |
| | 2174 | tgagctggtgaagcctggggcttcagtgaagatatcctgcaagactt | |
| | | ctggatacacattcactgaatacaccatacactgggtgaagcagagc | |
| | | catggagagagccttgagtggattggaggtattaatccaaacgatgg | |
| | | tggtactatttacaaccagaagttcaagggcaaggccacattgactg | |
| | | tagacaagtcctccaaaacagcctacatggagctccgcagcctgaca | |
| | | tctgaggattctgcagtcttttactgtgcaagagggccctccgccag | |
| | | gtttccttactggggccaagggactctggtcactgtctctgcagcca | |
| | | aaacgacacccccatctgtctatccactggcccctggatctgctgcc | |
| | | caaactaactccatggtgaccctgggatgcctggtcaagggctattt | |
| | | ccctgagccagtgacagtgacctggaactctggatccctgtccagcg | |
| | | gtgtgcacaccttcccagctgtcctgcagtctgacctctacactctg | |
| | | agcagctcagtgactgtcccctccagcacctggcccagcgagaccgt | |
| | | cacctgcaacgttgcccacccggccagcagcaccaaggtggacaaga | |
| | | aaattgtgcccagggattgtggttgtaagccttgcatatgtacagtc | |
| | | ccagaagtatcatctgtcttcatcttccccccaaagcccaaggatgt | |
| | | gctcaccattactctgactcctaaggtcacgtgtgttgtggtagaca | |
| | | tcagcaaggatgatcccgaggtccagttcagctggtttgtagatgat | |
| | | gtggaggtgcacacagctcagacgcaaccccgggaggagcagttcaa | |
| | | cagcactttccgctcagtcagtgaacttcccatcatgcaccaggact | |
| | | ggctcaatggcaaggagttcaaatgcagggtcaacagtgcagctttc | |
| | | cctgcccccatcgagaaaaccatctccaaaaccaaaggcagaccgaa | |
| | | ggctccacaggtgtacaccattccacctcccaaggagcagatggcca | |
| | | aggataaagtcagtctgacctgcatgataacagacttcttccctgaa | |
| | | gacattactgtggagtggcagtggaatgggcagccagcggagaacta | |
| | | caagaacactcagcccatcatggacacagatggctcttacttcgtct | |
| | | acagcaagctcaatgtgcagaagagcaactgggaggcaggaaatact | |
| | | ttcacctgctctgtgttacatgagggcctgcacaaccaccatactga | |
| | | gaagagcctctcccactctcctggtagaaagaggcgagagggcagag | |
| | | gaagtcttctaacatgcggtgacgtggaggagaatcccggccctatg | |
| | | atgagtcctgcccagttcctgtttctgttagtgctctggattcggga | |
| | | aaccaacggtgatgttgtgatgacccagactccactcactttgtcgg | |
| | | ttaccattggacaaccagcctccatctcttgcaagtcaagtcagagc | |
| | | ctcttagatagtgatggaaagacatatttgaattggttgttacagag | |
| | | gccaggccagtctccaaagcgcctaatctatctggtgtctaaactgg | |
| | | actctggagtccctgacagattcactggcagtggatcagggacagat | |
| | | ttcacactgaaaattagcagagtggaggctgaggatttgggagttta | |
| | | ttattgctggcaaggtacacattttcctcggacgttcggtggaggca | |
| | | ccaagctggaaatcaaacgggctgatgctgcaccaactgtatccatc | |
| | | ttcccaccatccagtgagcagttaacatctggaggtgcctcagtcgt | |
| | | gtgcttcttgaacaacttctaccccaaagacatcaatgtcaagtgga | |
| | | agattgatggcagtgaacgacaaaatggcgtcctgaacagttggact | |
| | | gatcaggacagcaaagacagcacctacagcatgagcagcaccctcac | |
| | | gttgaccaaggacgagtatgaacgacataacagctatacctgtgagg | |
| | | ccactcacaagacatcaacttcacccattgtcaagagcttcaacagg | |
| | | aatgagtgttag | |
| Ab249 | PHF-1 | MGWSWIFLFLLSGTTGVLSEVQLQQSGPELVKPGASVKISCKTSGYT | 1988 |
| | Furin 2A- | FTEYTIHWVKQSHGESLEWIGGINPNDGGTIYNQKFKGKATLTVDKS | |
| | 720 | SKTAYMELRSLTSEDSAVFYCARGPSARFPYWGQGTLVTVSAAKTTP | |
| | | PSVYPLAPGSAAQTNSMVTLGCLVKGYFPEPVTVTWNSGSLSSGVHT | |
| | | FPAVLQSDLYTLSSSVTVPSSTWPSETVTCNVAHPASSTKVDKKIVP | |
| | | RDCGCKPCICTVPEVSSVFIFPPKPKDVLTITLTPKVTCVVVDISKD | |
| | | DPEVQFSWFVDDVEVHTAQTQPREEQFNSTFRSVSELPIMHQDWLNG | |
| | | KEFKCRVNSAAFPAPIEKTISKTKGRPKAPQVYTIPPPKEQMAKDKV | |
| | | SLTCMITDFFPEDITVEWQWNGQPAENYKNTQPIMDTDGSYFVYSKL | |
| | | NVQKSNWEAGNTFTCSVLHEGLHNHHTEKSLSHSPGRKRREGRGSLL | |
| | | TCGDVEENPGPMMSPAQFLFLLVLWIRETNGDVVMTQTPLTLSVTIG | |
| | | QPASISCKSSQSLLDSDGKTYLNWLLQRPGQSPKRLIYLVSKLDSGV | |
| | | PDRFTGSGSGTDFTLKISRVEAEDLGVYYCWQGTHFPRTFGGGTKLE | |
| | | IKRADAAPTVSIFPPSSEQLTSGGASVVCFLNNFYPKDINVKWKIDG | |
| | | SERQNGVLNSWTDQDSKDSTYSMSSTLTLTKDEYERHNSYTCEATHK | |
| | | TSTSPIVKSFNRNEC | |
| Ab250 | PHF-1 | gaggagccaccatgggctggagctggatcttcctgttcctgctgagc | 1989 |
| | optimized | ggcaccaccggcgtgctgagcgaggtgcagctgcagcagagcggccc | |
| | seq | cgagctggtgaagcccggcgccagcgtgaagatcagctgcaagacca | |
| | | gcggctacaccttcaccgagtacaccatccactgggtgaagcagagc | |
| | | cacggcgagagcctggagtggatcggcggcatcaaccccaacgacgg | |
| | | cggcaccatctacaaccagaagttcaagggcaaggccaccctgaccg | |
| | | tggacaagagcagcaagaccgcctacatggagctgcggagcctgacc | |
| | | agcgaggacagcgccgtgttctactgcgctcggggccccagcgcccg | |
| | | gttcccctactggggccagggcaccctggtgaccgtgagcgccgcca | |
| | | agaccacaccacccagcgtgtacccactggctccaggcagcgctgcc | |
| | | caaaccaacagcatggtgaccctgggctgcctggtgaagggctactt | |
| | | ccccgagcccgtgaccgtgacctggaacagcggcagcctgagcagcg | |
| | | gcgtgcacaccttcccagctgtgctccagtccgacctgtacaccctg | |
| | | agcagcagcgtgaccgtgcccagcagcacctggcccagcgagaccgt | |
| | | gacctgcaacgtggcccaccccgccagcagcaccaaggtggacaaga | |
| | | agatcgtgccccgggactgcggctgcaagccctgcatctgcaccgtg | |
| | | cctgaggtctccagcgtgttcatcttcccccccaagcccaaggacgt | |
| | | gctgaccatcaccctgacccctaaagtcacctgcgtggtggtggaca | |
| | | tcagcaaggacgaccccgaggtgcagttcagctggttcgtggacgac | |
| | | gtggaggtgcacaccgcccagacccagccccgggaggagcagttcaa | |
| | | cagcaccttccggagcgtgagcgagctgcccatcatgcaccaggact | |
| | | ggctgaacggcaaggagttcaagtgccgggtgaacagcgccgccttc | |
| | | cccgcccccatcgagaagaccatcagcaagaccaagggccgacccaa | |
| | | ggccccacaggtgtacaccatcccaccacccaaggagcagatggcca | |
| | | aggacaaagtcagcctgacctgcatgatcaccgacttcttccccgag | |
| | | gacatcaccgtggagtggcagtggaacggccagccagccgagaacta | |
| | | caagaacacccagcccatcatggacaccgacggcagctacttcgtgt | |
| | | acagcaagctgaacgtgcagaagagcaactgggaggccggcaacacc | |
| | | ttcacctgcagcgtgctgcacgagggcctgcacaaccaccacaccga | |
| | | gaagagcctgagccacagccccggacggaagcggcgcgagggacggg | |
| | | gcagcctgctgacctgcggcgacgtggaggagaacccaggccccatg | |
| | | atgagcccagcccagttcctgttcctgctggtgctgtggatccggga | |
| | | gaccaacggcgacgtggtgatgacccagaccccactgaccctgagcg | |
| | | tgaccatcggccagcccgccagcatcagctgcaagagcagccagagc | |
| | | ctgctggacagcgacggcaagacctacctgaactggctgctgcagcg | |
| | | gccaggccagagccccaagcggctgatctatctcgtcagcaagctgg | |
| | | acagcggcgtgcccgaccggttcaccggcagcggcagcggcaccgac | |
| | | ttcaccctgaagatcagccgggtggaggccgaggacctgggcgtgta | |
| | | ctactgctggcagggcacccactttccccggaccttcggcggcggca | |
| | | ccaagctggagatcaagcgggccgacgccgccccaacagtcagcatc | |
| | | tttccaccatcctccgagcagctcaccagcggcggcgccagcgtggt | |
| | | gtgcttcctgaacaacttctaccccaaggacatcaacgtgaagtgga | |
| | | agatcgacggcagcgagcggcagaacggcgtgctgaacagctggacc | |
| | | gaccaggacagcaaggacagcacctacagcatgagcagcaccctgac | |
| | | cctgaccaaggacgagtacgagcggcacaacagctacacctgcgagg | |
| | | ccacccacaagaccagcaccagccccatcgtgaagagcttcaaccgg | |
| | | aacgagtgctga | |
| Ab251 | PT3 VH | aagctggtggagagcggcggcgacctggtgaagcctggcggcagcct | 2241 |
| | | gaagctgagctgcgccgccagcggcttcaccttcagcagctacgcca | |
| | | tgagctgggtgagacagaaccctgagaagagactggagtgggtggcc | |
| | | agcatcagcaagggggcaacacctactaccctaacagcgtgaaggg | |
| | | cagattcaccatcagcagagacaacgccagaaacatcctgtacctgc | |
| | | agatgagcagcctgagaagcgaggacaccgccctgtactactgcgcc | |
| | | agaggctggggcgactacggctggttcgcctactggggccaggtgac | |
| | | cctggtgaccgtgagcgcc | |
| Ab252 | VH | gccaaaacgacacccccatctgtctatccactggcccctggatctgc | 2242 |
| | (constant- | tgcccaaactaactccatggtgaccctgggatgcctggtcaagggct | |
| | 309) | atttccctgagccagtgacagtgacctggaactctggatccctgtcc | |
| | | agcggtgtgcacaccttcccagctgtcctgcagtctgacctctacac | |
| | | tctgagcagctcagtgactgtcccctccagcacctggcccagcgaga | |
| | | ccgtcacctgcaacgttgcccacccggccagcagcaccaaggtggac | |
| | | aagaaaattgtgcccagggattgtggt | |
| Ab253 | VH | gccaaaacgacacccccatctgtctatccactggcccctggatctgc | 2243 |
| | (constant- | tgcccaaactaactccatggtgaccctgggatgcctggtcaagggct | |
| | 291) | atttccctgagccagtgacagtgacctggaactctggatccctgtcc | |
| | | agcggtgtgcacaccttcccagctgtcctgcagtctgacctctacac | |
| | | tctgagcagctcagtgactgtcccctccagcacctggcccagcgaga | |
| | | ccgtcacctgcaacgttgcccacccggccagcagcaccaaggtggac | |
| | | aagaaaatt | |
| Ab254 | PHF1 VH- | atggcgacgggttcaagaacttccctacttcttgcatttggcctgct | 2169 |
| | 435 | ttgtttgccgtggttacaggagggctcggcagctgccgaggtccagc | |
| | | tgcaacagtctggacctgagctggtgaagcctggggcttcagtgaag | |
| | | atatcctgcaagacttctggatacacattcactgaatacaccataca | |
| | | ctgggtgaagcagagccatggagagagccttgagtggattggaggta | |
| | | ttaatccaaacgatggtggtactatttacaaccagaagttcaagggc | |
| | | aaggccacattgactgtagacaagtcctccaaaacagcctacatgga | |
| | | gctccgcagcctgacatctgaggattctgcagtcttttactgtgcaa | |
| | | gagggccctccgccaggtttccttactggggccaagggactctggtc | |
| | | actgtctctgca | |
| Ab255 | PHF1 VH- | atgggatggagctggatctttctctttctcctgtcaggaactacagg | 2170 |
| | 408 | tgtcctctctgaggtccagctgcaacagtctggacctgagctggtga | |
| | | agcctggggcttcagtgaagatatcctgcaagacttctggatacaca | |
| | | ttcactgaatacaccatacactgggtgaagcagagccatggagagag | |
| | | ccttgagtggattggaggtattaatccaaacgatggtggtactattt | |
| | | acaaccagaagttcaagggcaaggccacattgactgtagacaagtcc | |
| | | tccaaaacagcctacatggagctccgcagcctgacatctgaggattc | |
| | | tgcagtcttttactgtgcaagagggccctccgccaggtttccttact | |
| | | ggggccaagggactctggtcactgtctctgca | |

In some embodiments, the antibody molecules comprise the HCDR1, HCDR2, HCDR3, LCDR1, LCDR2, and/or LCDR3 sequences, the VH and/or VL sequences, or the heavy and/or light chain sequences, of an antibody molecule described in Tables 7 and 8, e.g., an art-recognized anti-tau antibody molecule selected from the group consisting of: 7295-M6, 7297-2M1, 7298-M1, 7299-M2, 7299-M5, 7299-M9, hC10.2, C10.2, C8.3, C5.2, D1.2, IPN001, IPN002, MC1, PT1, PT3, PHF1, and AT8.

In some embodiments, the ant-tau antibody molecule is IPN002, a fragment thereof, or a variant thereof. In some embodiments, the anti-tau antibody molecule comprises the HCDR1, HCDR2, and HCDR3 sequences, and/or the LCDR1, LCDR2, and LCDR3 sequences, of IPN002. In some embodiments, the antibody molecule comprises the HCDR1, HCDR2, and HCDR3 sequences set forth in SEQ ID NOs: 1777, 1778, and 1779, respectively, and LCDR1, LCDR2, and LCDR3 sequences set forth in SEQ ID NOs: 1889, 1890, and 1891, respectively. In some embodiments, one or more of the CDRs (or collectively all of the CDRs) have one, two, three, four, five or more changes, e.g., amino acid substitutions, insertions, or deletions, relative to the amino acid sequence of SEQ ID NOs: 1777, 1778, 1779, 1889, 1890, and/or 1891. In some embodiments, the antibody molecule comprises the VH and/or VL sequences of IPN002. In some embodiments, the antibody molecule comprises a VH comprising the amino acid sequence of SEQ ID NO: 1822 and/or a VL comprising the amino acid sequence of SEQ ID NO: 1941, or a sequence substantially identical, e.g., having at least about 70%, 75%, 80%, 85%, 90%, 92%, 95%, 97%, 98%, or 99% sequence identity thereto, or having at least one, two, or three modifications (e.g., substitutions, e.g., conservative substitutions), but not more than 30, 20, or 10 modifications (e.g., substitutions, e.g., conservative substitutions) relative to the VH and/or VL sequence. In some embodiments, the anti-tau antibody molecule is an antibody which competes for binding to tau (e.g., human tau) with IPN002. In some embodiments, the anti-tau antibody molecule is an antibody which binds to the same epitope as, substantially the same epitope as, an epitope that overlaps with, or an epitope that substantially overlaps with, IPN002.

In some embodiments, the ant-tau antibody molecule is AT8, a fragment thereof, or a variant thereof. In some embodiments, the anti-tau antibody molecule comprises the HCDR1, HCDR2, and HCDR3 sequences, and/or the LCDR1, LCDR2, and LCDR3 sequences, of AT8. In some embodiments, one or more of the CDRs (or collectively all of the CDRs) have one, two, three, four, five or more changes, e.g., amino acid substitutions, insertions, or deletions, relative to the amino acid sequence of one or more of the CDRs of AT8. In some embodiments, the antibody molecule comprises the VH and/or VL sequences of AT8, or a sequence substantially identical, e.g., having at least about 70%, 75%, 80%, 85%, 90%, 92%, 95%, 97%, 98%, or 99% sequence identity thereto, or having at least one, two, or three modifications (e.g., substitutions, e.g., conservative substitutions), but not more than 30, 20, or 10 modifications (e.g., substitutions, e.g., conservative substitutions) relative to the VH and/or VL sequence of AT8. In some embodiments, the anti-tau antibody molecule is an antibody which competes for binding to tau (e.g., human tau) with AT08. In some embodiments, the anti-tau antibody molecule is an antibody which binds to the same epitope as, substantially the same epitope as, an epitope that overlaps with, or an epitope that substantially overlaps with, AT08.

In some embodiments, the ant-tau antibody molecule is PT3, a fragment thereof, or a variant thereof. In some embodiments, the anti-tau antibody molecule comprises the HCDR1, HCDR2, and HCDR3 sequences, and/or the LCDR1, LCDR2, and LCDR3 sequences, of PT3. In some embodiments, the antibody molecule comprises the HCDR1, HCDR2, and HCDR3 sequences set forth in SEQ ID NOs: 1790, 1792, and 1794, respectively, and LCDR1, LCDR2, and LCDR3 sequences set forth in SEQ ID NOs: 1908, 1910, and 1912, respectively. In some embodiments, one or more of the CDRs (or collectively all of the CDRs) have one, two, three, four, five or more changes, e.g., amino acid substitutions, insertions, or deletions, relative to the amino acid sequence of SEQ ID NO: 1790, 1792, 1794, 1908, 1910, and/or 1912. In some embodiments, the antibody molecule comprises the VH and/or VL sequences of PT3. In some embodiments, the antibody molecule comprises a VH comprising the amino acid sequence of SEQ ID NO: 1849 and/or a VL comprising the amino acid sequence of SEQ ID NO: 1970, or a sequence substantially identical, e.g., having at least about 70%, 75%, 80%, 85%, 90%, 92%, 95%, 97%, 98%, or 99% sequence identity thereto, or having at least one, two, or three modifications (e.g., substitutions, e.g., conservative substitutions), but not more than 30, 20, or 10 modifications (e.g., substitutions, e.g., conservative substitutions) relative to the VH and/or VL sequence. In some embodiments, the anti-tau antibody molecule is an antibody which competes for binding to tau (e.g., human tau) with PT3. In some embodiments, the anti-tau antibody molecule is an antibody which binds to the same epitope as, substantially the same epitope as, an epitope that overlaps with, or an epitope that substantially overlaps with, PT3.

In some embodiments, the ant-tau antibody molecule is UCB, a fragment thereof, or a variant thereof. In some embodiments, the anti-tau antibody molecule comprises the HCDR1, HCDR2, and HCDR3 sequences, and/or the LCDR1, LCDR2, and LCDR3 sequences, of UCB. In some embodiments, one or more of the CDRs (or collectively all of the CDRs) have one, two, three, four, five or more changes, e.g., amino acid substitutions, insertions, or deletions, relative to the amino acid sequence of one or more of the CDRs of UCB. In some embodiments, the antibody molecule comprises the VH and/or VL sequences of UCB, or a sequence substantially identical, e.g., having at least about 70%, 75%, 80%, 85%, 90%, 92%, 95%, 97%, 98%, or 99% sequence identity thereto, or having at least one, two, or three modifications (e.g., substitutions, e.g., conservative substitutions), but not more than 30, 20, or 10 modifications (e.g., substitutions, e.g., conservative substitutions) relative to the VH and/or VL sequence. In some embodiments, the anti-tau antibody molecule is an antibody which competes for binding to tau (e.g., human tau) with UCB. In some embodiments, the anti-tau antibody molecule is an antibody which binds to the same epitope as, substantially the same epitope as, an epitope that overlaps with, or an epitope that substantially overlaps with, UCB.

In some embodiments, the ant-tau antibody molecule is PT76, a fragment thereof, or a variant thereof. In some embodiments, the anti-tau antibody molecule comprises the HCDR1, HCDR2, and HCDR3 sequences, and/or the LCDR1, LCDR2, and LCDR3 sequences, of PT76. In some embodiments, one or more of the CDRs (or collectively all of the CDRs) have one, two, three, four, five or more changes, e.g., amino acid substitutions, insertions, or deletions, relative to the amino acid sequence of one or more of the CDRs of PT76. In some embodiments, the antibody molecule comprises the VH and/or VL sequences of PT76, or a sequence substantially identical, e.g., having at least about 70%, 75%, 80%, 85%, 90%, 92%, 95%, 97%, 98%, or 99% sequence identity thereto, or having at least one, two, or three modifications (e.g., substitutions, e.g., conservative substitutions), but not more than 30, 20, or 10 modifications (e.g., substitutions, e.g., conservative substitutions) relative to the VH and/or VL sequence of PT76. In some embodiments, the anti-tau antibody molecule is an antibody which competes for binding to tau (e.g., human tau) with PT76. In some embodiments, the anti-tau antibody molecule is an antibody which binds to the same epitope as, substantially the same epitope as, an epitope that overlaps with, or an epitope that substantially overlaps with, PT76.

In some embodiments, the ant-tau antibody molecule is PHF1, a fragment thereof, or a variant thereof. In some embodiments, the anti-tau antibody molecule comprises the HCDR1, HCDR2, and HCDR3 sequences, and/or the LCDR1, LCDR2, and LCDR3 sequences, of PHF1. In some embodiments, one or more of the CDRs (or collectively all of the CDRs) have one, two, three, four, five or more changes, e.g., amino acid substitutions, insertions, or deletions, relative to the amino acid sequence of one or more of the CDRs of PHF1. In some embodiments, the antibody molecule comprises the VH and/or VL sequences of PHF1. In some embodiments, the antibody molecule comprises a VH comprising the amino acid sequence of SEQ ID NO: 1844 and/or a VL comprising the amino acid sequence of SEQ ID NO: 1964, or a sequence substantially identical, e.g., having at least about 70%, 75%, 80%, 85%, 90%, 92%, 95%, 97%, 98%, or 99% sequence identity thereto, or having at least one, two, or three modifications (e.g., substitutions, e.g., conservative substitutions), but not more than 30, 20, or 10 modifications (e.g., substitutions, e.g., conservative substitutions) relative to the VH and/or VL sequence. In some embodiments, the anti-tau antibody molecule is an antibody which competes for binding to tau (e.g., human tau) with PHF1. In some embodiments, the anti-tau antibody molecule is an antibody which binds to the same epitope as, substantially the same epitope as, an epitope that overlaps with, or an epitope that substantially overlaps with, PHF1.

In some embodiments, the ant-tau antibody molecule is C10.2, a fragment thereof, or a variant thereof. In some embodiments, the anti-tau antibody molecule comprises the HCDR1, HCDR2, and HCDR3 sequences, and/or the LCDR1, LCDR2, and LCDR3 sequences, of C10.2. In some embodiments, the antibody molecule comprises the HCDR1, HCDR2, and HCDR3 sequences set forth in SEQ ID NOs: 1761, 1762, and 1763, respectively, and LCDR1, LCDR2, and LCDR3 sequences set forth in SEQ ID NOs: 1880, 1881, and 1882, respectively. In some embodiments, one or more of the CDRs (or collectively all of the CDRs) have one, two, three, four, five or more changes, e.g., amino acid substitutions, insertions, or deletions, relative to the amino acid sequence of SEQ ID NO: 1761, 1762, 1763, 1880, 1881, and/or 1882. In some embodiments, the antibody molecule comprises the VH and/or VL sequences of C10.2. In some embodiments, the antibody molecule comprises a VH comprising the amino acid sequence of SEQ ID NO: 1803 and/or a VL comprising the amino acid sequence of SEQ ID NO: 1919, or a sequence substantially identical, e.g., having at least about 70%, 75%, 80%, 85%, 90%, 92%, 95%, 97%, 98%, or 99% sequence identity thereto, or having at least one, two, or three modifications (e.g., substitutions, e.g., conservative substitutions), but not more than 30, 20, or 10 modifications (e.g., substitutions, e.g., conservative substitutions) relative to the VH and/or VL sequence. In some embodiments, the anti-tau antibody molecule is an antibody which competes for binding to tau (e.g., human tau) with C10.2. In some embodiments, the anti-tau antibody molecule is an antibody which binds to the same epitope as, substantially the same epitope as, an epitope that overlaps with, or an epitope that substantially overlaps with, C10.2.

In some embodiments, the anti-tau antibody molecule may target any of the antigenic regions or epitopes described in International Publication WO2021/211753, the contents of which are herein incorporated by reference in their entirety. For instance, in some embodiments, the anti-tau antibody molecule comprises HCDR1, HCDR2, and/or HCDR3, and LCDR1, LCDR2, and/or LCDR3 sequences encompassed by the consensus CDR sequences described in Table 9, for example, the CDR sequences of V0022, V0023, and V0024 (as shown in Tables 10-13).

TABLE 9
Consensus CDRs for exemplary anti-tau antibodies
V0022, V0023 and V0024

| SEQ ID NO | Description | Sequence |
|---|---|---|
| SEQ ID NO: 1631 | HCDR1 | GX₁TFTX₂X₃, wherein |
| (Chothia) | | X₁ is For Y; |
| | | X₂ is R or I; and |
| | | X₃ is Y or F |
| SEQ ID NO: 1551 | HCDR2 | NPNNGG |
| (Chothia) | | |
| SEQ ID NO: 1552 | HCDR3 | GTGTGAMDY |
| (Chothia) | | |
| SEQ ID NO: 1632 | LCDR1 | RSSQSLVHX₁NGX₂TX₃LY, wherein |
| (Chothia) | | X₁ is N or S; |
| | | X₂ is I or N; and |
| | | X₃ is Y or H |
| SEQ ID NO: 1633 | LCDR2 | RVSX₁RFS, wherein |
| (Chothia) | | X₁ is N or S |
| SEQ ID NO: 1566 | LCDR3 | FQGTHVPRT |
| (Chothia) | | |
| SEQ ID NO: 1634 | HCDR1 | X₁X₂WMH, wherein |
| (Kabat) | | X₁ is R or I; and |
| | | X₂ is Y or F |
| SEQ ID NO: 1635 | HCDR2 | X₁INPNNGGX₂DX₃NEX₄FKX₅, wherein |
| (Kabat) | | X₁ is N or K; |
| | | X₂ is T or G; |
| | | X₃ is F, Y, or N; |
| | | X₄ is K or R; and |
| | | X₅ is N or S |
| SEQ ID NO: 1552 | HCDR3 | GTGTGAMDY |
| (Kabat) | | |
| SEQ ID NO: 1636 | LCDR1 | RSSQSLVHX₁NGX₂TX₃LY, wherein |
| (Kabat) | | X₁ is N or S; |
| | | X₂ is I or N; and |
| | | X₃ is Y or H |
| SEQ ID NO: 1633 | LCDR2 | RVSX₁RFS, wherein |
| (Kabat) | | X₁ is N or S |
| SEQ ID NO: 1566 | LCDR3 | FQGTHVPRT |
| (Kabat) | | |
| SEQ ID NO: 1637 | HCDR1 | GX₁TFTX₂X₃W, wherein |
| (IMGT) | | X₁ is For Y; |
| | | X₂ is R or I; and |
| | | X₃ is Y or F |
| SEQ ID NO: 1638 | HCDR2 | INPNNGG X₁, wherein |
| (IMGT) | | X₁ is T or G |
| SEQ ID NO: 1557 | HCDR3 | ARGTGTGAMDY |
| (IMGT) | | |
| SEQ ID NO: 1639 | LCDR1 | QSLVHX₁INGX₂TX₃, wherein |
| (IMGT) | | X₁ is N or S; |
| | | X₂ is I or N; and |
| | | X₃ is Y or H |
| SEQ ID NO: 1569 | LCDR2 | RVS |
| (IMGT) | | |
| SEQ ID NO: 1566 | LCDR3 | FQGTHVPRT |
| (IMGT) | | |

In some embodiments, the anti-tau antibody molecule comprises at least one, two, or three complementarity determining regions (CDRs) from a VH comprising an amino acid sequence in Tables 9-16, or is encoded by a nucleic acid sequence in Table 13 or 14; or a sequence substantially identical (e.g., having at least about 70%, 75%, 80%, 85%, 90%, 92%, 95%, 97%, 98%, or 99% sequence identity) to any of the aforesaid sequences. In some embodiments, one or more of the CDRs (or collectively all of the CDRs) have one, two, three, four, five or more changes, e.g., amino acid substitutions, insertions, or deletions, relative to the amino acid sequence shown in Table 9-16, or encoded by a nucleotide sequence shown in Table 13 or 14. In some embodiments, the encoded anti-tau antibody molecule includes a substitution in a HCDR, e.g., one or more substitutions in a HCDR1, HCDR2 and/or HCDR3.

In some embodiments, the anti-tau antibody molecule comprises at least one, two, or three complementarity determining regions (CDRs) from a VL comprising an amino acid sequence in Tables 9-16, or is encoded by a nucleic acid sequence in Table 13 or 14; or a sequence substantially identical (e.g., having at least about 80%, 85%, 90%, 92%, 95%, 97%, 98%, or 99% sequence identity) to any of the aforesaid sequences. In some embodiments, one or more of the CDRs (or collectively all of the CDRs) have one, two, three, four, five or more changes, e.g., amino acid substitutions, insertions, or deletions, relative to the amino acid sequence shown in Tables 9-16, or encoded by a nucleotide sequence shown in Table 13 or 14. In some embodiments, the anti-tau antibody molecule includes a substitution in a LCDR, e.g., one or more substitutions in a LCDR1, LCDR2, and/or LCDR3 of the light chain.

In some embodiments, the anti-tau antibody molecule comprises at least one, two, three, four, five or six CDRs (or collectively all of the CDRs) from a VH and VL comprising an amino acid sequence shown in Table 9-16, or is encoded by a nucleotide sequence shown in Table 13 or 14. In some embodiments, one or more of the CDRs (or collectively all of the CDRs) have one, two, three, four, five, six or more changes, e.g., amino acid substitutions, insertions, or deletions, relative to the CDRs shown in Table 9-16, or encoded by a nucleotide sequence shown in Table 13 or 14.

In some embodiments, the anti-tau antibody molecule comprises all three CDRs from a VH, all three CDRs from a VL, or both (e.g., all six CDRs from a VH and a VL) comprising an amino acid sequence shown in Table 9-16, or is encoded by a nucleotide sequence shown in Table 13 or 14.

In some embodiments, the anti-tau antibody molecule comprises a VH from an antibody chosen from V0001-V0065, V1001-V1005, or V2001-V2005, e.g., as described in Table 13 and WO2021/211753, the contents of which are hereby incorporated by reference, or VY011, VY007, VY004, VY006, VY018, VY003, VY016, VY017, VY012, VY009, VY010, VY022, VY001, VY019, VY020, VY005, VY002, VY014, VY008, or VY013, e.g., as described in Tables X1-X3 and WO2022/132923 (see, e.g., Examples 3, 4, and 6-9 of WO2022/132923), the contents of which are hereby incorporated by reference, or a sequence substantially identical (e.g., having at least about 70%, 75%, 80%, 85%, 90%, 92%, 95%, 97%, 98%, or 99% sequence identity) to any of the aforesaid sequences. In some embodiments, the VH comprises an amino acid sequence having at least one, two, or three modifications (e.g., substitutions, e.g., conservative substitutions), but not more than 30, 20, or 10 modifications (e.g., substitutions, e.g., conservative substitutions), relative to an amino acid sequence of a VH provided in Table 13 and WO2021/211753 or Tables 14-16 and WO2022/132923.

In some embodiments, the nucleotide sequence encoding the anti-tau antibody molecule comprises the nucleotide sequence of a VH from an antibody molecule chosen from V0001-V0065, V1001-V1005, or V2001-V2005, e.g., as described in Table 13 and WO2021/211753, or VY011, VY007, VY004, VY006, VY018, VY003, VY016, VY017, VY012, VY009, VY010, VY022, VY001, VY019, VY020, VY005, VY002, VY014, VY008, or VY013, e.g., as described in Table 14, or a nucleotide sequence substantially identical (e.g., having at least about 70%, 75%, 80%, 85%, 90%, 92%, 95%, 97%, 98%, or 99% sequence identity) to any of the aforesaid sequences.

In some embodiments, the anti-tau antibody molecule comprises a VL from an antibody molecule chosen from V0001-V0065, V1001-V1005, or V2001-V2005, e.g., as described in Table 13 and WO2021/211753, or VY011, VY007, VY004, VY006, VY018, VY003, VY016, VY017, VY012, VY009, VY010, VY022, VY001, VY019, VY020, VY005, VY002, VY014, VY008, or VY013, e.g., as described in Tables 14-16, or a sequence substantially identical (e.g., having at least about 70%, 75%, 80%, 85%, 90%, 92%, 95%, 97%, 98%, or 99% sequence identity) to any of the aforesaid sequences. In some embodiments, the VL comprises an amino acid sequence having at least one, two, or three modifications (e.g., substitutions, e.g., conservative substitutions), but not more than 30, 20, or 10 modifications (e.g., substitutions, e.g., conservative substitutions), relative to an amino acid sequence of a VL provided in Table 13 and WO2021/211753.

In some embodiments, the nucleotide sequence encoding the anti-tau antibody molecule comprises the nucleotide sequence of a VL from an antibody chosen from V0001-V0065, V1001-V1005, or V2001-V2005, e.g., as described in Table 13 and WO2021/211753, or VY011, VY007, VY004, VY006, VY018, VY003, VY016, VY017, VY012, VY009, VY010, VY022, VY001, VY019, VY020, VY005, VY002, VY014, VY008, or VY013, e.g., as described in Table 14 and WO2022/132923, or a nucleotide sequence substantially identical (e.g., having at least about 70%, 75%, 80%, 85%, 90%, 92%, 95%, 97%, 98%, or 99% sequence identity) to any of the aforesaid sequences.

In some embodiments, the anti-tau antibody molecule comprises a VH and a VL from an antibody chosen from V0001-V0065, V1001-V1005, or V2001-V2005, e.g., as described in Table 13 and WO2021/211753, or VY011, VY007, VY004, VY006, VY018, VY003, VY016, VY017, VY012, VY009, VY010, VY022, VY001, VY019, VY020, VY005, VY002, VY014, VY008, or VY013, e.g., as described in Tables 14-16 and WO2022/132923, or a sequence substantially identical (e.g., having at least about 70%, 75%, 80%, 85%, 90%, 92%, 95%, 97%, 98%, or 99% sequence identity) to any of the aforesaid sequences. In some embodiments, the anti-tau antibody molecule comprises a VH comprising an amino acid sequence having at least one, two, or three modifications (e.g., substitutions, e.g., conservative substitutions), but not more than 30, 20, or 10 modifications (e.g., substitutions, e.g., conservative substitutions), relative to an amino acid sequence of a VH provided in Table 13 and WO2021/211753 or Tables 14-16 and WO2022/132923; and a VL comprising an amino acid sequence having at least one, two, or three modifications (e.g., substitutions, e.g., conservative substitutions), but not more than 30, 20, or 10 modifications (e.g., substitutions, e.g., conservative substitutions), relative to an amino acid sequence of a VL provided in Table 13 and WO2021/211753 or Tables 14-16 and WO2022/132923.

In some embodiments, the anti-tau antibody molecule comprises HCDR1, HCDR2, HCDR3, LCDR1, LCDR2, and LCDR3 sequences set forth in (a) SEQ ID NOs: 1631, 1551, 1552, 1632, 1633, and 1566, respectively, (b) SEQ ID NOs: 1634, 1635, 1552, 1636, 1633, and 1566, respectively, (c) SEQ ID NOs: 1637, 1638, 1557, 1639, 1569, and 1566, respectively, (d) SEQ ID NOs: 1550, 1551, 1552, 1564, 1565, and 1566, respectively, (e) SEQ ID NO: 1553, 1554, 1552, 1567, 1565, and 1566, respectively, (f) SEQ ID NOs: 1555, 1556, 1557, 1568, 1569, and 1566, respectively, (g) SEQ ID NOs: 1575, 1551, 1552, 1585, 1565, and 1566, respectively, (h) SEQ ID NOs: 1576, 1577, 1552, 1585, 1565, and 1566, respectively, (i) SEQ ID NOs: 1578, 1579, 1557, 1586, 1569, and 1566, respectively, (j) SEQ ID NOs: 1593, 1551, 1552, 1601, 1602, and 1566, respectively, (k) SEQ ID NO: 1594, 1595, 1552, 1601, 1602, and 1566, respectively, (1) SEQ ID NOs: 1596, 1556, 1557, 1603, 1569, and 1566, respectively, (m) SEQ ID NOs: 1608, 1609, 1610, 1621, 1622, and 1623, respectively, (n) SEQ ID NO: 1611, 1612, 1610, 1621, 1622, and 1623, respectively, (o) SEQ ID NOs: 1613, 1614, 1615, 1624, 1625, and 1623, respectively, (p) SEQ ID NOs: 1525, 1526, 1527, 1539, 1540, and 1541, respectively, (q) SEQ ID NO: 1528, 1529, 1527, 1539, 1540, and 1541, respectively, (r) SEQ ID NOs: 1530, 1531, 1532, 1542, 1543, and 1541, respectively, (s) SEQ ID NOs: 1500, 1501, 1502, 1514, 1515, and 1516, respectively, (t) SEQ ID NO: 1503, 1504, 1502, 1514, 1515, and 1516, respectively, (u) SEQ ID NOs: 1505, 1506, 1507, 1517, 1518, and 1516, respectively, (v) SEQ ID NOs: 1777, 1778, 1779, 1889, 1890, and 1891, respectively, (w) SEQ ID NOs: 1790, 1792, 1794, 1908, 1910, and 1912, respectively, (x) SEQ ID NOs: 1761, 1762, 1763, 1880, 1881, and 1882, respectively, (y) SEQ ID NOs: 2200, 2201, 2202, 2207, 2208, and 2209, respectively, (z) SEQ ID NOs: 2232, 2233, 2234, 2238, 2239, and 2240, respectively, (za) SEQ ID NOs: 2248, 2219, 2249, 2207, 2208, and 2224, respectively, (zb) SEQ ID NOs: 2218, 2219, 2220, 2207, 2208, and 2224, respectively, (zc) SEQ ID NOs: 2200, 2257, 2258, 2262, 2263, and 2264, respectively, (zd) SEQ ID NOs: 2415, 2416, 2417, 2420, 2421, and 2422, respectively, (ze) SEQ ID NOs: 2273, 2274, 2275, 2207, 2208, and 2277, respectively, (zf) SEQ ID NOs: 2284, 2285, 2286, 2289, 2290, and 2291, respectively, (zg) SEQ ID NOs: 2284, 2299, 2300, 2302, 2303, and 2304, respectively, (zh) SEQ ID NOs: 2313, 2314, 2315, 2319, 2320, and 2321, respectively, (zi) SEQ ID NOs: 2330, 2331, 2332, 2262, 2336, and 2337, respectively, (zj) SEQ ID NOs: 2345, 2346, 2347, 2351, 2352, and 2353, respectively, (zk) SEQ ID NOs: 2345, 2346, 2347, 2361, 2362, and 2363, respectively, (zl) SEQ ID NOs: 2371, 2372, 2373, 2207, 2208, and 2224, respectively, (zm) SEQ ID NOs: 2381, 2382, 2383, 2262, 2263, and 2385, respectively, (zn) SEQ ID NOs: 2284, 2392, 2393, 2396, 2239, and 2397, respectively, (zo) SEQ ID NOs: 2284, 2392, 2393, 2404, 2405, and 2406, respectively, (zp) SEQ ID NOs: 2430, 2431, 2432, 2435, 2436, and 2437, respectively, (zq) SEQ ID NOs: 2445, 2446, 2447, 2452, 2421, and 2453, respectively, or (zr) SEQ ID NOs: 2459, 2460, 2461, 2465, 2466, and 2467, respectively.

In some embodiments, the anti-tau antibody molecule comprises VH and/or VL sequences set forth in: (a) SEQ ID NOs: 1562 and 1573, respectively; (b) SEQ ID NOs: 1512 and 1523, respectively; (c) SEQ ID NOs: 1537 and 1548, respectively; (d) SEQ ID NOs: 1583 and 1591, respectively; (e) SEQ ID NOs: 1599 and 1606, respectively; (f) SEQ ID NOs: 1619 and 1629, respectively; (g) SEQ ID NOs: 1822 and 1941, respectively; (h) SEQ ID NOs: 1849 and 1970, respectively; (i) SEQ ID NOs: 1844 and 1964, respectively; (j) SEQ ID NOs: 1803 and 1919, respectively; (k) SEQ ID NOs: 2214 and 2215, respectively; (1) SEQ ID NOs: 2244 and 2245, respectively; (m) SEQ ID NOs: 2253 and 2254, respectively; (n) SEQ ID NOs: 2228 and 2229, respectively; (o) SEQ ID NOs: 2269 and 2270, respectively; (p) SEQ ID NOs: 2426 and 2427, respectively; (q) SEQ ID NOs: 2280 and 2281, respectively; (r) SEQ ID NOs: 2295 and 2296, respectively; (s) SEQ ID NOs: 2309 and 2310, respectively; (t) SEQ ID NOs: 2326 and 2327, respectively; (u) SEQ ID NOs: 2341 and 2342, respectively; (v) SEQ ID NOs: 2357 and 2358, respectively; (w) SEQ ID NOs: 2367 and 2368, respectively; (x) SEQ ID NOs: 2377 and 2378, respectively; (y) SEQ ID NOs: 2388 and 2389, respectively; (z) SEQ ID NOs: 2400 and 2401, respectively; (za) SEQ ID NOs: 2411 and 2412, respectively; (zb) SEQ ID NOs: 2441 and 2442, respectively; (zc) SEQ ID NOs: 2455 and 2456, respectively; or (zd) SEQ ID NOs: 2470 and 2471, respectively.

In some embodiments, the anti-tau antibody molecule comprises a heavy chain constant region, e.g., a human IgG1, IgG2, IgG3, or IgG4 constant regions, or a murine IgG1, IgG2A, IgG2B, IgG2C, or IgG3 constant regions. In some embodiments, the heavy chain constant comprises an amino acid sequence set forth in Table 17, or a sequence substantially identical (e.g., having at least about 70%, 75%, 80%, 85%, 90%, 92%, 95%, 97%, 98%, or 99% sequence identity) to any of the aforesaid sequences. In some embodiments, a nucleic acid encoding the heavy chain constant region comprises a nucleotide sequence set forth in Table 17, or a nucleotide sequence substantially identical (e.g., having at least about 70%, 75%, 80%, 85%, 90%, 92%, 95%, 97%, 98%, or 99% sequence identity) to any of the aforesaid sequences.

In some embodiments, the anti-tau antibody molecule (e.g., the anti-tau antibody molecule described in any of Tables 9-16) comprises a light chain constant region, e.g., a kappa light chain constant region, e.g., a human kappa or lambda light chain constant region or a murine kappa or lambda light chain constant region. In some embodiments, the light chain constant comprises an amino acid sequence set forth in Table 17, or a sequence substantially identical (e.g., having at least about 70%, 75%, 80%, 85%, 90%, 92%, 95%, 97%, 98%, or 99% sequence identity) to any of the aforesaid sequences. In some embodiments, nucleic acid encoding the light chain constant region comprises a nucleotide sequence set forth in Table 17, or a nucleotide sequence substantially identical (e.g., having at least about 70%, 75%, 80%, 85%, 90%, 92%, 95%, 97%, 98%, or 99% sequence identity) to any of the aforesaid sequences.

In some embodiments, the anti-tau antibody molecule (e.g., the anti-tau antibody molecule described in any of Tables 9-16) comprises a heavy chain constant region and a light chain constant region. In some embodiments, the heavy chain constant region and the light chain constant region comprise an amino acid sequence set forth in Table 17, or a sequence substantially identical (e.g., having at least about 80%, 85%, 90%, 92%, 95%, 97%, 98%, or 99% sequence identity) thereto. In some embodiments, the nucleotide sequence encoding the anti-tau antibody molecule comprises the nucleotide sequence of a heavy chain constant region and the nucleotide sequence of a kappa or lambda light chain constant region. In some embodiments, the nucleotide sequence encoding the heavy chain constant region and light chain constant region comprise a nucleotide sequence set forth in Table 17, or a nucleotide sequence substantially identical (e.g., having at least about 80%, 85%, 90%, 92%, 95%, 97%, 98%, or 99% sequence identity) thereto.

TABLE 10
CDR sequences of selected antibodies based on Kabat numbering system

| Ab ID | HC CDR1 | HC CDR2 | HC CDR3 | LC CDR1 | LC CDR2 | LC CDR3 |
|---|---|---|---|---|---|---|
| V0004 | NYWMH | RIDPNSGGT | DFDV | RASGNIH | NAKTLPD | QHFWSTPLT |
| | (SEQ ID | RYNEKFKN | (SEQ ID | NYLA | (SEQ ID | (SEQ ID |
| | NO: 1503) | (SEQ ID | NO: | (SEQ ID | NO: 1515) | NO: 1516) |
| | | NO: 1504) | 1502) | NO: | | |
| | | | | 1514) | | |
| V0009 | DYYMS | LIRNKAKGF | DINY | KSSQSLL | WASSRES | QQYYSYPRT |
| | (SEQ ID | TTEYSASVK | (SEQ ID | YSTNQEN | (SEQ ID | (SEQ ID |
| | NO: 1528) | G | NO: | YLA | NO: 1540) | NO: 1541) |
| | | (SEQ ID | 1527) | (SEQ ID | | |
| | | NO: 1529) | | NO: | | |
| | | | | 1539) | | |
| V0022 | RYWMH | NINPNNGGT | GTGTGAM | RSSQSLV | RVSNRFS | FQGTHVPRT |
| | (SEQ ID | DFNEKFKN | DY | HNNGITY | (SEQ ID | (SEQ ID |
| | NO: 1553) | (SEQ ID | (SEQ ID | LY | NO: 1565) | NO: 1566) |
| | | NO: 1554) | NO: | (SEQ ID | | |
| | | | 1552) | NO: | | |
| | | | | 1567) | | |
| V0023 | IFWMH | KINPNNGGG | GTGTGAM | RSSQSLV | RVSNRFS | FQGTHVPRT |
| | (SEQ ID | DYNEKFKS | DY | HSNGITH | (SEQ ID | (SEQ ID |
| | NO: 1576) | (SEQ ID | (SEQ ID | LY | NO: 1565) | NO: 1566) |
| | | NO: 1577) | NO: | (SEQ ID | | |
| | | | 1552) | NO: | | |
| | | | | 1585) | | |
| V0024 | RFWMH | NINPNNGGT | GTGTGAM | RSSQSLV | RVSSRFS | FQGTHVPRT |
| | (SEQ ID | DNNERFKS | DY | HSNGNTH | (SEQ ID | (SEQ ID |
| | NO: 1594) | (SEQ ID | (SEQ ID | LY | NO: 1602) | NO: 1566) |
| | | NO: 1595) | NO: | (SEQ ID | | |
| | | | 1552) | NO: | | |
| | | | | 1601) | | |
| V0052 | TSAMGVS | HIYWDDDKR | RRRGYGM | KASQSVS | YASNRCT | QQDYRSPLT |
| | (SEQ ID | YNPSLKS | DY | NDVA | (SEQ ID | (SEQ ID |
| | NO: 1611) | (SEQ ID | (SEQ ID | (SEQ ID | NO: 1622) | NO: 1623) |
| | | NO: 1612) | NO: | NO: | | |
| | | | 1610) | 1621) | | |

TABLE 11
CDR sequences of selected antibodies based on Chothia numbering system

| Ab ID | HC CDR1 | HC CDR2 | HC CDR3 | LC CDR1 | LC CDR2 | LC CDR3 |
|---|---|---|---|---|---|---|
| V0004 | DYTFTNY | DPNSGG | DFDV | RASGNIH | NAKTLPD | QHFWSTPLT |
| | (SEQ ID | (SEQ ID | (SEQ ID | NYLA | (SEQ ID | (SEQ ID |
| | NO: 1500) | NO: 1501) | NO: | (SEQ ID | NO: 1515) | NO: 1516) |
| | | | 1502) | NO: | | |
| | | | | 1514) | | |
| V0009 | GFTFTDY | RNKAKGFT | DINY | KSSQSLL | WASSRES | QQYYSYPRT |
| | (SEQ ID | (SEQ ID | (SEQ ID | YSTNQEN | (SEQ ID | (SEQ ID |
| | NO: 1525) | NO: 1526) | NO: | YLA | NO: 1540) | NO: 1541) |
| | | | 1527) | (SEQ ID | | |
| | | | | NO: | | |
| | | | | 1539) | | |
| V0022 | GFTFTRY | NPNNGG | GTGTGAM | RSSQSLV | RVSNRFS | FQGTHVPRT |
| | (SEQ ID | (SEQ ID | DY | HNNGITY | (SEQ ID | (SEQ ID |
| | NO: 1550) | NO: 1551) | (SEQ ID | LY | NO: 1565) | NO: 1566) |
| | | | NO: | (SEQ ID | | |
| | | | 1552) | NO: | | |
| | | | | 1564) | | |
| V0023 | GYTFTIF | NPNNGG | GTGTGAM | RSSQSLV | RVSNRFS | FQGTHVPRT |
| | (SEQ ID | (SEQ ID | DY | HSNGITH | (SEQ ID | (SEQ ID |
| | NO: 1575) | NO: 1551) | (SEQ ID | LY | NO: 1565) | NO: 1566) |
| | | | NO: | (SEQ ID | | |
| | | | 1552) | NO: | | |
| | | | | 1585) | | |
| V0024 | GYTFTRF | NPNNGG | GTGTGAM | RSSQSLV | RVSSRFS | FQGTHVPRT |
| | (SEQ ID | (SEQ ID | DY | HSNGNTH | (SEQ ID | (SEQ ID |
| | NO: 1593) | NO: 1551) | (SEQ ID | LY | NO: 1602) | NO: 1566) |
| | | | NO: | (SEQ ID | | |
| | | | 1552) | NO: | | |
| | | | | 1601) | | |
| V0052 | GFSLSTSA | YWDDD | RRRGYGM | KASQSVS | YASNRCT | QQDYRSPLT |
| | M | (SEQ ID | DY | NDVA | (SEQ ID | (SEQ ID |
| | (SEQ ID | NO: 1609) | (SEQ ID | (SEQ ID | NO: 1622) | NO: 1623) |
| | NO: 1608) | | NO: | NO: | | |
| | | | 1610) | 1621) | | |

TABLE 12
CDR sequences of selected antibodies based on IMGT numbering system

| Ab ID | HC CDR1 | HC CDR2 | HC CDR3 | LC CDR1 | LC CDR2 | LC CDR3 |
|---|---|---|---|---|---|---|
| V0004 | DYTFTNYW | IDPNSGGT | AGDFDV | GNIHNY | NAK | QHFWSTPLT |
| | (SEQ ID | (SEQ ID | (SEQ ID | (SEQ ID | (SEQ ID | (SEQ ID |
| | NO: 1505) | NO: 1506) | NO: 1507) | NO: | NO: 1518) | NO: 1516) |
| | | | | 1517) | | |
| V0009 | GFTFTDYY | IRNKAKGFT | VRDINY | QSLLYST | WAS | QQYYSYPRT |
| | (SEQ ID | T | (SEQ ID | NQENY | (SEQ ID | (SEQ ID |
| | NO: 1530) | (SEQ ID | NO: 1532) | (SEQ ID | NO: 1543) | NO: 1541) |
| | | NO: 1531) | | NO: | | |
| | | | | 1542) | | |
| V0022 | GFTFTRYW | INPNNGGT | ARGTGTGA | QSLVHNN | RVS | FQGTHVPRT |
| | (SEQ ID | (SEQ ID | MDY | GITY | (SEQ ID | (SEQ ID |
| | NO: 1555) | NO: 1556) | (SEQ ID | (SEQ ID | NO: 1569) | NO: 1566) |
| | | | NO: 1557) | NO: | | |
| | | | | 1568) | | |
| V0023 | GYTFTIFW | INPNNGGG | ARGTGTGA | QSLVHSN | RVS | FQGTHVPRT |
| | (SEQ ID | (SEQ ID | MDY | | (SEQ ID | (SEQ ID |
| | NO: 1578) | NO: 1579) | (SEQ ID | GITH | NO: 1569) | NO: 1566) |
| | | | NO: 1557) | (SEQ ID | | |
| | | | | NO: | | |
| | | | | 1586) | | |
| V0024 | GYTFTRFW | INPNNGGT | ARGTGTGA | QSLVHSN | RVS | FQGTHVPRT |
| | (SEQ ID | (SEQ ID | MDY | GNTH | (SEQ ID | (SEQ ID |
| | NO: 1596) | NO: 1556) | (SEQ ID | (SEQ ID | NO: 1569) | NO: 1566) |
| | | | NO: 1557) | NO: | | |
| | | | | 1603) | | |
| V0052 | GFSLSTSA | IYWDDDK | ARRRRGYG | QSVSND | YAS | QQDYRSPLT |
| | MG | (SEQ ID | MDY | (SEQ ID | (SEQ ID | (SEQ ID |
| | (SEQ ID | NO: 1614) | (SEQ ID | NO: | NO: 1625) | NO: 1623) |
| | NO: 1613) | | NO: 1615) | 1624) | | |

TABLE 13
Exemplary anti-tau antibodies

| | SEQ | | |
|---|---|---|---|
| | ID | | |
| Ab ID | NO | Description | Sequence |
| V0004 | 1500 | HC CDR1 | DYTFTNY |
| | | (Chothia) | |
| | 1501 | HC CDR2 | DPNSGG |
| | | (Chothia) | |
| | 1502 | HC CDR3 | DFDV |
| | | (Chothia) | |
| | 1503 | HC CDR1 | NYWMH |
| | | (Kabat) | |
| | 1504 | HC CDR2 | RIDPNSGGTRYNEKFKN |
| | | (Kabat) | |
| | 1502 | HC CDR3 | DFDV |
| | | (Kabat) | |
| | 1505 | HC CDR1 | DYTFTNYW |
| | | (IMGT) | |
| | 1506 | HC CDR2 | IDPNSGGT |
| | | (IMGT) | |
| | 1507 | HC CDR3 | AGDFDV |
| | | (IMGT) | |
| | 1508 | FR H1 | QVQLQQPGAELVKPGASVKLSCKAS |
| | | (corresponding to the | |
| | | Chothia CDR | |
| | | definition) | |
| | 1509 | FR H2 | WMHWVKQRPGRGLEWIGRI |
| | | (corresponding to the | |
| | | Chothia CDR | |
| | | definition) | |
| | 1510 | FR H3 | TRYNEKFKNKATLTVDKPSSTAYMHLSSLTSEDSAVYYC |
| | | (corresponding to the | AG |
| | | Chothia CDR | |
| | | definition) | |
| | 1511 | FR H4 | WGTGTTVTVSS |
| | | (corresponding to the | |
| | | Chothia CDR | |
| | | definition) | |
| | 1512 | VH | QVQLQQPGAELVKPGASVKLSCKASDYTFTNYWMHWVKQ |
| | | | RPGRGLEWIGRIDPNSGGTRYNEKFKNKATLTVDKPSST |
| | | | AYMHLSSLTSEDSAVYYCAGDFDVWGTGTTVTVSS |
| | 1513 | DNA VH | CAGGTCCAACTGCAGCAGCCTGGGGCTGAGCTTGTGAAG |
| | | | CCTGGGGCTTCAGTGAAGCTGTCCTGCAAGGCTTCTGAC |
| | | | TACACCTTCACCAACTACTGGATGCACTGGGTGAAGCAG |
| | | | AGGCCTGGACGAGGCCTTGAGTGGATAGGAAGGATTGAT |
| | | | CCTAATAGTGGTGGTACTAGGTACAATGAGAAGTTCAAG |
| | | | AACAAGGCCACACTGACTGTTGACAAACCCTCCAGCACA |
| | | | GCCTACATGCATCTCAGCAGCCTGACATCTGAGGACTCT |
| | | | GCGGTCTATTATTGTGCAGGGGACTTCGATGTCTGGGGC |
| | | | ACAGGGACCACGGTCACCGTCTCCTCA |
| | 1514 | LC CDR1 | RASGNIHNYLA |
| | | (Chothia) | |
| | 1515 | LC CDR2 | NAKTLPD |
| | | (Chothia) | |
| | 1516 | LC CDR3 | QHFWSTPLT |
| | | (Chothia) | |
| | 1514 | LC CDR1 | RASGNIHNYLA |
| | | (Kabat) | |
| | 1515 | LC CDR2 | NAKTLPD |
| | | (Kabat) | |
| | 1516 | LC CDR3 | QHFWSTPLT |
| | | (Kabat) | |
| | 1517 | LC CDR1 | GNIHNY |
| | | (IMGT) | |
| | 1518 | LC CDR2 | NAK |
| | | (IMGT) | |
| | 1516 | LC CDR3 | QHFWSTPLT |
| | | (IMGT) | |
| | 1519 | FR L1 | DIQMTQSPASLSASVGETVTITC |
| | | (corresponding to the | |
| | | Chothia CDR | |
| | | definition) | |
| | 1520 | FR L2 | WYQQRQGKSPQLLVY |
| | | (corresponding to the | |
| | | Chothia CDR | |
| | | definition) | |
| | 1521 | FR L3 | GVPSRFSGSGSGTQYSLKINSLQPEDFGSYCC |
| | | (corresponding to the | |
| | | Chothia CDR | |
| | | definition) | |
| | 1522 | FR L4 | FGAGTKLELK |
| | | (corresponding to the | |
| | | Chothia CDR | |
| | | definition) | |
| | 1523 | VL | DIQMTQSPASLSASVGETVTITCRASGNIHNYLAWYQQR |
| | | | QGKSPQLLVYNAKTLPDGVPSRFSGSGSGTQYSLKINSL |
| | | | QPEDFGSYCCQHFWSTPLTFGAGTKLELK |
| | 1524 | DNA VL | GACATCCAGATGACTCAGTCTCCAGCCTCCCTATCTGCA |
| | | | TCTGTGGGAGAAACTGTCACCATCACATGTCGAGCAAGT |
| | | | GGGAATATTCACAATTATTTAGCATGGTATCAGCAGAGA |
| | | | CAGGGAAAATCTCCTCAGCTCCTGGTCTATAATGCAAAA |
| | | | ACCTTACCAGATGGTGTGCCATCAAGGTTCAGTGGCAGT |
| | | | GGATCAGGAACACAATATTCTCTCAAGATCAACAGCCTG |
| | | | CAGCCTGAAGATTTTGGGAGTTATTGCTGTCAACATTTT |
| | | | TGGAGTACTCCGCTCACGTTCGGTGCTGGGACCAAGTTG |
| | | | GAGCTGAAA |
| V0009 | 1525 | HC CDR1 | GFTFTDY |
| | | (Chothia) | |
| | 1526 | HC CDR2 | RNKAKGFT |
| | | (Chothia) | |
| | 1527 | HC CDR3 | DINY |
| | | (Chothia) | |
| | 1528 | HC CDR1 | DYYMS |
| | | (Kabat) | |
| | 1529 | HC CDR2 | LIRNKAKGFTTEYSASVKG |
| | | (Kabat) | |
| | 1527 | HC CDR3 | DINY |
| | | (Kabat) | |
| | 1530 | HC CDR1 | GFTFTDYY |
| | | (IMGT) | |
| | 1531 | HC CDR2 | IRNKAKGFTT |
| | | (IMGT) | |
| | 1532 | HC CDR3 | VRDINY |
| | | (IMGT) | |
| | 1533 | FR H1 | EVQLVESGGALVQPGGSLSLSCAAS |
| | | (corresponding to the | |
| | | Chothia CDR | |
| | | definition) | |
| | 1534 | FR H2 | YMSWVRQPPGKALEWLALI |
| | | (corresponding to the | |
| | | Chothia CDR | |
| | | definition) | |
| | 1535 | FR H3 | TEYSASVKGRFTISRDNSQSILLFQMNDLRADDSATYYC |
| | | (corresponding to the | VR |
| | | Chothia CDR | |
| | | definition) | |
| | 1536 | FR H4 | WGQGTTLTVSS |
| | | (corresponding to the | |
| | | Chothia CDR | |
| | | definition) | |
| | 1537 | VH | EVQLVESGGALVQPGGSLSLSCAASGFTFTDYYMSWVRQ |
| | | | PPGKALEWLALIRNKAKGFTTEYSASVKGRFTISRDNSQ |
| | | | SILLFQMNDLRADDSATYYCVRDINYWGQGTTLTVSS |
| | 1538 | DNA VH | GAGGTGCAGCTGGTGGAGTCTGGAGGAGCCTTGGTACAG |
| | | | CCTGGGGGTTCTCTGAGTCTCTCCTGTGCAGCTTCTGGA |
| | | | TTCACCTTCACTGATTACTACATGAGCTGGGTCCGCCAG |
| | | | CCTCCAGGGAAGGCACTTGAGTGGCTGGCTTTGATTAGA |
| | | | AACAAAGCTAAAGGTTTCACAACAGAATACAGTGCATCT |
| | | | GTGAAGGGTCGGTTCACCATCTCCAGAGATAATTCCCAA |
| | | | AGCATCCTCCTTTTTCAAATGAATGACCTGAGAGCTGAC |
| | | | GACAGTGCCACTTATTACTGTGTAAGAGATATAAACTAC |
| | | | TGGGGCCAAGGCACCACTCTCACAGTCTCCTCA |
| | 1539 | LC CDR1 | KSSQSLLYSTNQENYLA |
| | | (Chothia) | |
| | 1540 | LC CDR2 | WASSRES |
| | | (Chothia) | |
| | 1541 | LC CDR3 | QQYYSYPRT |
| | | (Chothia) | |
| | 1539 | LC CDR1 | KSSQSLLYSTNQENYLA |
| | | (Kabat) | |
| | 1540 | LC CDR2 | WASSRES |
| | | (Kabat) | |
| | 1541 | LC CDR3 | QQYYSYPRT |
| | | (Kabat) | |
| | 1542 | LC CDR1 | QSLLYSTNQENY |
| | | (IMGT) | |
| | 1543 | LC CDR2 | WAS |
| | | (IMGT) | |
| | 1541 | LC CDR3 | QQYYSYPRT |
| | | (IMGT) | |
| | 1544 | FR L1 | DIVMSQSPSSLAVSVGEKVTMSC |
| | | (corresponding to the | |
| | | Chothia CDR | |
| | | definition) | |
| | 1545 | FR L2 | WYQQKPGQSPKLLIY |
| | | (corresponding to the | |
| | | Chothia CDR | |
| | | definition) | |
| | 1546 | FR L3 | GVPDRFTGSGSGTDFTLTISSVKAEDLAVYYC |
| | | (corresponding to the | |
| | | Chothia CDR | |
| | | definition) | |
| | 1547 | FR L4 | FGGGTKLEIK |
| | | (corresponding to the | |
| | | Chothia CDR | |
| | | definition) | |
| | 1548 | VL | DIVMSQSPSSLAVSVGEKVTMSCKSSQSLLYSTNQENYL |
| | | | AWYQQKPGQSPKLLIYWASSRESGVPDRFTGSGSGTDFT |
| | | | LTISSVKAEDLAVYYCQQYYSYPRTFGGGTKLEIK |
| | 1549 | DNA VL | GACATTGTGATGTCACAGTCTCCATCCTCCCTAGCTGTG |
| | | | TCAGTTGGAGAGAAGGTTACTATGAGCTGCAAGTCCAGT |
| | | | CAGAGCCTTTTATATAGTACCAATCAAGAGAACTACTTG |
| | | | GCCTGGTACCAGCAGAAACCAGGGCAGTCTCCTAAACTG |
| | | | CTGATTTACTGGGCATCCTCTAGGGAATCTGGGGTCCCT |
| | | | GATCGCTTTACAGGCAGTGGATCTGGGACAGATTTCACT |
| | | | CTCACCATCAGCAGTGTGAAGGCTGAAGACCTGGCAGTT |
| | | | TATTACTGTCAGCAATATTATAGCTATCCTCGGACGTTC |
| | | | GGTGGAGGCACCAAGCTGGAAATCAAA |
| V0022 | 1550 | HC CDR1 | GFTFTRY |
| | | (Chothia) | |
| | 1551 | HC CDR2 | NPNNGG |
| | | (Chothia) | |
| | 1552 | HC CDR3 | GTGTGAMDY |
| | | (Chothia) | |
| | 1553 | HC CDR1 | RYWMH |
| | | (Kabat) | |
| | 1554 | HC CDR2 | NINPNNGGTDFNEKFKN |
| | | (Kabat) | |
| | 1552 | HC CDR3 | GTGTGAMDY |
| | | (Kabat) | |
| | 1555 | HC CDR1 | GFTFTRYW |
| | | (IMGT) | |
| | 1556 | HC CDR2 | INPNNGGT |
| | | (IMGT) | |
| | 1557 | HC CDR3 | ARGTGTGAMDY |
| | | (IMGT) | |
| | 1558 | FR H1 | QVQLQQPGTELVKPGSSVNLSCKAS |
| | | (corresponding to the | |
| | | Chothia CDR | |
| | | definition) | |
| | 1559 | FR H2 | WMHWVKERPGHGLEWIGNI |
| | | (corresponding to the | |
| | | Chothia CDR | |
| | | definition) | |
| | 1560 | FR H3 | TDFNEKFKNKATLTVHKSSTTVFIQLSSLISEDSAVYYC |
| | | (corresponding to the | AR |
| | | Chothia CDR | |
| | | definition) | |
| | 1561 | FR H4 | WGQGTSVTVSS |
| | | (corresponding to the | |
| | | Chothia CDR | |
| | | definition) | |
| | 1562 | VH | QVQLQQPGTELVKPGSSVNLSCKASGFTFTRYWMHWVKE |
| | | | RPGHGLEWIGNINPNNGGTDFNEKFKNKATLTVHKSSTT |
| | | | VFIQLSSLTSEDSAVYYCARGTGTGAMDYWGQGTSVTVS |
| | | | S |
| | 1563 | DNA VH | CAGGTCCAACTGCAGCAGCCTGGGACTGAACTGGTGAAG |
| | | | CCTGGGTCTTCAGTGAACCTGTCCTGCAAGGCTTCTGGC |
| | | | TTCACCTTCACCAGGTACTGGATGCACTGGGTGAAGGAG |
| | | | AGGCCTGGACATGGCCTTGAGTGGATTGGAAATATTAAT |
| | | | CCTAACAATGGTGGTACTGACTTCAATGAGAAGTTCAAG |
| | | | AACAAGGCCACACTGACTGTACACAAGTCCTCCACCACA |
| | | | GTCTTCATCCAACTCAGCAGCCTGACATCTGAGGACTCT |
| | | | GCGGTCTATTATTGTGCAAGAGGAACTGGGACGGGAGCT |
| | | | ATGGACTACTGGGGTCAAGGAACCTCAGTCACCGTCTCC |
| | | | TCA |
| | 1564 | LC CDR1 | RSSQSLVHNNGITYLY |
| | | (Chothia) | |
| | 1565 | LC CDR2 | RVSNRFS |
| | | (Chothia) | |
| | 1566 | LC CDR3 | FQGTHVPRT |
| | | (Chothia) | |
| | 1567 | LC CDR1 | RSSQSLVHNNGITYLY |
| | | (Kabat) | |
| | 1565 | LC CDR2 | RVSNRFS |
| | | (Kabat) | |
| | 1566 | LC CDR3 | FQGTHVPRT |
| | | (Kabat) | |
| | 1568 | LC CDR1 | QSLVHNNGITY |
| | | (IMGT) | |
| | 1569 | LC CDR2 | RVS |
| | | (IMGT) | |
| | 1566 | LC CDR3 | FQGTHVPRT |
| | | (IMGT) | |
| | 1570 | FR L1 | DVVMTQTPLSLPVSLGDQASISC |
| | | (corresponding to the | |
| | | Chothia CDR | |
| | | definition) | |
| | 1571 | FR L2 | WYLQKPGQSPKLLIY |
| | | (corresponding to the | |
| | | Chothia CDR | |
| | | definition) | |
| | 1572 | FR L3 | GVPDRFGGSGSGTDFTLKISRVEAEDLGVYFC |
| | | (corresponding to the | |
| | | Chothia CDR | |
| | | definition) | |
| | 1547 | FR L4 | FGGGTKLEIK |
| | | (corresponding to the | |
| | | Chothia CDR | |
| | | definition) | |
| | 1573 | VL | DVVMTQTPLSLPVSLGDQASISCRSSQSLVHNNGITYLY |
| | | | WYLQKPGQSPKLLIYRVSNRFSGVPDRFGGSGSGTDFTL |
| | | | KISRVEAEDLGVYFCFQGTHVPRTFGGGTKLEIK |
| | 1574 | DNA VL | GATGTTGTGATGACCCAAACTCCACTCTCCCTGCCTGTC |
| | | | AGTCTTGGAGATCAAGCTTCCATCTCTTGCAGATCTAGT |
| | | | CAGAGCCTTGTACACAACAATGGAATCACCTATTTATAT |
| | | | TGGTACCTGCAGAAGCCAGGCCAGTCTCCAAAGCTCCTG |
| | | | ATCTACAGGGTTTCCAACCGATTTTCTGGGGTCCCAGAC |
| | | | AGGTTCGGTGGCAGTGGATCAGGGACAGATTTCACACTC |
| | | | AAGATCAGCAGAGTGGAGGCTGAGGATCTGGGAGTTTAT |
| | | | TTCTGCTTTCAAGGTACACATGTTCCTCGGACGTTCGGT |
| | | | GGAGGCACCAAGCTGGAAATCAAA |
| V0023 | 1575 | HC CDR1 | GYTFTIF |
| | | (Chothia) | |
| | 1551 | HC CDR2 | NPNNGG |
| | | (Chothia) | |
| | 1552 | HC CDR3 | GTGTGAMDY |
| | | (Chothia) | |
| | 1576 | HC CDR1 | IFWMH |
| | | (Kabat) | |
| | 1577 | HC CDR2 | KINPNNGGGDYNEKFKS |
| | | (Kabat) | |
| | 1552 | HC CDR3 | GTGTGAMDY |
| | | (Kabat) | |
| | 1578 | HC CDR1 | GYTFTIFW |
| | | (IMGT) | |
| | 1579 | HC CDR2 | INPNNGGG |
| | | (IMGT) | |
| | 1557 | HC CDR3 | ARGTGTGAMDY |
| | | (IMGT) | |
| | 1580 | FR H1 | QVQLQQPGTELVKPGASVKLSCKAS |
| | | (corresponding to the | |
| | | Chothia CDR | |
| | | definition) | |
| | 1581 | FR H2 | WMHWVKQRPGHGLEWIGKI |
| | | (corresponding to the | |
| | | Chothia CDR | |
| | | definition) | |
| | 1582 | FR H3 | GDYNEKFKSKATLTVDKSSTTAYLQLSSLISEDSAVYYC |
| | | (corresponding to the | AR |
| | | Chothia CDR | |
| | | definition) | |
| | 1561 | FR H4 | WGQGTSVTVSS |
| | | (corresponding to the | |
| | | Chothia CDR | |
| | | definition) | |
| | 1583 | VH | QVQLQQPGTELVKPGASVKLSCKASGYTFTIFWMHWVKQ |
| | | | RPGHGLEWIGKINPNNGGGDYNEKFKSKATLTVDKSSTT |
| | | | AYLQLSSLTSEDSAVYYCARGTGTGAMDYWGQGTSVTVS |
| | | | S |
| | 1584 | DNA VH | CAGGTCCAACTGCAGCAGCCTGGGACTGAACTGGTGAAG |
| | | | CCTGGGGCTTCAGTGAAGCTGTCCTGCAAGGCTTCTGGC |
| | | | TACACCTTCACCATCTTCTGGATGCACTGGGTGAAGCAG |
| | | | AGGCCTGGACATGGCCTTGAGTGGATTGGAAAGATTAAT |
| | | | CCTAACAATGGAGGTGGTGACTACAATGAGAAATTCAAG |
| | | | AGTAAGGCCACATTGACTGTAGACAAATCCTCCACCACA |
| | | | GCCTACTTGCAGCTCAGCAGCCTGACATCTGAGGACTCT |
| | | | GCGGTCTATTATTGTGCAAGAGGAACTGGGACGGGAGCT |
| | | | ATGGACTACTGGGGTCAAGGAACCTCAGTCACCGTCTCC |
| | | | TCA |
| | 1585 | LC CDR 1 | RSSQSLVHSNGITHLY |
| | | (Chothia) | |
| | 1565 | LC CDR2 | RVSNRFS |
| | | (Chothia) | |
| | 1566 | LC CDR3 | FQGTHVPRT |
| | | (Chothia) | |
| | 1585 | LC CDR1 | RSSQSLVHSNGITHLY |
| | | (Kabat) | |
| | 1565 | LC CDR2 | RVSNRFS |
| | | (Kabat) | |
| | 1566 | LC CDR3 | FQGTHVPRT |
| | | (Kabat) | |
| | 1586 | LC CDR1 | QSLVHSNGITH |
| | | (IMGT) | |
| | 1569 | LC CDR2 | RVS |
| | | (IMGT) | |
| | 1566 | LC CDR3 | FQGTHVPRT |
| | | (IMGT) | |
| | 1587 | FR L1 | DVVMTQTPLSLPVSLGDHASISC |
| | | (corresponding to the | |
| | | Chothia CDR | |
| | | definition) | |
| | 1588 | FR L2 | WYLQRPGQTPKLLIY |
| | | (corresponding to the | |
| | | Chothia CDR | |
| | | definition) | |
| | 1589 | FR L3 | GVPDRFSGSGSGTDFTLKISSVEAEDLGVYFC |
| | | (corresponding to the | |
| | | Chothia CDR | |
| | | definition) | |
| | 1590 | FR L4 | FGGGTKLEIE |
| | | (corresponding to the | |
| | | Chothia CDR | |
| | | definition) | |
| | 1591 | VL | DVVMTQTPLSLPVSLGDHASISCRSSQSLVHSNGITHLY |
| | | | WYLQRPGQTPKLLIYRVSNRFSGVPDRFSGSGSGTDFTL |
| | | | KISSVEAEDLGVYFCFQGTHVPRTFGGGTKLEIE |
| | 1592 | DNA VL | GATGTTGTGATGACCCAAACTCCACTCTCCCTGCCTGTC |
| | | | AGTCTTGGAGATCACGCTTCCATCTCTTGCAGATCTAGT |
| | | | CAGAGCCTTGTACACAGCAATGGAATCACCCATTTATAT |
| | | | TGGTACCTGCAGAGGCCAGGCCAGACTCCAAAGCTCCTG |
| | | | ATCTACAGGGTTTCCAACCGATTTTCTGGGGTCCCAGAC |
| | | | AGGTTCAGTGGCAGTGGATCAGGGACAGATTTCACACTC |
| | | | AAGATCAGCAGCGTGGAGGCTGAGGATCTGGGAGTTTAT |
| | | | TTCTGCTTTCAAGGTACACATGTTCCTCGGACGTTCGGT |
| | | | GGAGGCACCAAGCTGGAAATCGAA |
| V0024 | 1593 | HC CDR1 | GYTFTRF |
| | | (Chothia) | |
| | 1551 | HC CDR2 | NPNNGG |
| | | (Chothia) | |
| | 1552 | HC CDR3 | GTGTGAMDY |
| | | (Chothia) | |
| | 1594 | HC CDR1 | RFWMH |
| | | (Kabat) | |
| | 1595 | HC CDR2 | NINPNNGGTDNNERFKS |
| | | (Kabat) | |
| | 1552 | HC CDR3 | GTGTGAMDY |
| | | (Kabat) | |
| | 1596 | HC CDR1 | GYTFTRFW |
| | | (IMGT) | |
| | 1556 | HC CDR2 | INPNNGGT |
| | | (IMGT) | |
| | 1557 | HC CDR3 | ARGTGTGAMDY |
| | | (IMGT) | |
| | 1580 | FR H1 | QVQLQQPGTELVKPGASVKLSCKAS |
| | | (corresponding to the | |
| | | Chothia CDR | |
| | | definition) | |
| | 1597 | FR H2 | WMHWVKQRPGQGLEWIGNI |
| | | (corresponding to the | |
| | | Chothia CDR | |
| | | definition) | |
| | 1598 | FR H3 | TDNNERFKSKATLTVDRSSSTAYMQLSSLTSEDSAVYYC |
| | | (corresponding to the | AR |
| | | Chothia CDR | |
| | | definition) | |
| | 1561 | FR H4 | WGQGTSVTVSS |
| | | (corresponding to the | |
| | | Chothia CDR | |
| | | definition) | |
| | 1599 | VH | QVQLQQPGTELVKPGASVKLSCKASGYTFTRFWMHWVKQ |
| | | | RPGQGLEWIGNINPNNGGTDNNERFKSKATLTVDRSSST |
| | | | AYMQLSSLTSEDSAVYYCARGTGTGAMDYWGQGTSVTVS |
| | | | S |
| | 1600 | DNA VH | CAGGTCCAACTGCAGCAGCCTGGGACTGAACTGGTGAAG |
| | | | CCTGGGGCTTCAGTGAAGCTGTCCTGCAAGGCTTCTGGC |
| | | | TACACCTTCACCAGGTTCTGGATGCACTGGGTGAAGCAG |
| | | | AGGCCTGGACAAGGCCTTGAGTGGATTGGAAATATTAAT |
| | | | CCTAACAATGGTGGTACTGACAATAATGAGAGGTTCAAG |
| | | | AGCAAGGCCACACTGACTGTAGACAGATCCTCCAGCACA |
| | | | GCCTACATGCAGCTCAGCAGCCTGACATCTGAGGACTCT |
| | | | GCGGTCTATTATTGTGCAAGAGGAACTGGGACGGGAGCT |
| | | | ATGGACTACTGGGGTCAAGGAACCTCAGTCACCGTCTCC |
| | | | TCA |
| | 1601 | LC CDR 1 | RSSQSLVHSNGNTHLY |
| | | (Chothia) | |
| | 1602 | LC CDR2 | RVSSRFS |
| | | (Chothia) | |
| | 1566 | LC CDR3 | FQGTHVPRT |
| | | (Chothia) | |
| | 1601 | LC CDR1 | RSSQSLVHSNGNTHLY |
| | | (Kabat) | |
| | 1602 | LC CDR2 | RVSSRFS |
| | | (Kabat) | |
| | 1566 | LC CDR3 | FQGTHVPRT |
| | | (Kabat) | |
| | 1603 | LC CDR1 | QSLVHSNGNTH |
| | | (IMGT) | |
| | 1569 | LC CDR2 | RVS |
| | | (IMGT) | |
| | 1566 | LC CDR3 | FQGTHVPRT |
| | | (IMGT) | |
| | 1604 | FR L1 | DVVMTQTPLSLPVSLGDQASISC |
| | | (corresponding to the | |
| | | Chothia CDR | |
| | | definition) | |
| | 1571 | FR L2 | WYLQKPGQSPKLLIY |
| | | (corresponding to the | |
| | | Chothia CDR | |
| | | definition) | |
| | 1605 | FR L3 | GVPDRFSGSGSGTDFTLKISRVEAEDLGVYFC |
| | | (corresponding to the | |
| | | Chothia CDR | |
| | | definition) | |
| | 1547 | FR L4 | FGGGTKLEIK |
| | | (corresponding to the | |
| | | Chothia CDR | |
| | | definition) | |
| | 1606 | VL | DVVMTQTPLSLPVSLGDQASISCRSSQSLVHSNGNTHLY |
| | | | WYLQKPGQSPKLLIYRVSSRFSGVPDRFSGSGSGTDFTL |
| | | | KISRVEAEDLGVYFCFQGTHVPRTFGGGTKLEIK |
| | 1607 | DNA VL | GATGTGGTGATGACCCAAACTCCACTCTCCCTGCCTGTC |
| | | | AGTCTTGGAGATCAGGCTTCCATCTCTTGCAGATCTAGT |
| | | | CAGAGCCTTGTACACAGCAATGGAAACACCCATTTATAT |
| | | | TGGTACCTGCAGAAGCCAGGCCAGTCTCCAAAGCTCCTG |
| | | | ATCTACAGGGTTTCCAGCCGATTTTCTGGGGTCCCAGAC |
| | | | AGGTTCAGTGGCAGTGGATCAGGGACAGATTTCACACTC |
| | | | AAGATCAGCAGAGTGGAGGCTGAGGATCTGGGAGTTTAT |
| | | | TTCTGCTTTCAGGGTACACATGTTCCTCGGACGTTCGGT |
| | | | GGAGGCACCAAGCTGGAAATCAAA |
| V0052 | 1608 | HC CDR1 | GFSLSTSAM |
| | | (Chothia) | |
| | 1609 | HC CDR2 | YWDDD |
| | | (Chothia) | |
| | 1610 | HC CDR3 | RRRGYGMDY |
| | | (Chothia) | |
| | 1611 | HC CDR1 | TSAMGVS |
| | | (Kabat) | |
| | 1612 | HC CDR2 | HIYWDDDKRYNPSLKS |
| | | (Kabat) | |
| | 1610 | HC CDR3 | RRRGYGMDY |
| | | (Kabat) | |
| | 1613 | HC CDR1 | GFSLSTSAMG |
| | | (IMGT) | |
| | 1614 | HC CDR2 | IYWDDDK |
| | | (IMGT) | |
| | 1615 | HC CDR3 | ARRRRGYGMDY |
| | | (IMGT) | |
| | 1616 | FR H1 | QITLKESGPGILQSSQTLSLTCSFS |
| | | (corresponding to the | |
| | | Chothia CDR | |
| | | definition) | |
| | 1617 | FR H2 | GVSWIRQPSGEGLEWLAHI |
| | | (corresponding to the | |
| | | Chothia CDR | |
| | | definition) | |
| | 1618 | FR H3 | KRYNPSLKSRLTISKDTSRNQVFLKITSVDTADTATYYC |
| | | (corresponding to the | AR |
| | | Chothia CDR | |
| | | definition) | |
| | 1561 | FR H4 | WGQGTSVTVSS |
| | | (corresponding to the | |
| | | Chothia CDR | |
| | | definition) | |
| | 1619 | VH | QITLKESGPGILQSSQTLSLTCSFSGFSLSTSAMGVSWI |
| | | | RQPSGEGLEWLAHIYWDDDKRYNPSLKSRLTISKDTSRN |
| | | | QVFLKITSVDTADTATYYCARRRRGYGMDYWGQGTSVTV |
| | | | SS |
| | 1620 | DNA VH | CAGATTACTCTGAAAGAGTCTGGCCCTGGGATATTGCAG |
| | | | TCCTCCCAGACCCTCAGTCTGACTTGTTCTTTCTCTGGG |
| | | | TTTTCACTGAGCACTTCTGCTATGGGTGTGAGTTGGATT |
| | | | CGTCAGCCTTCAGGAGAGGGTCTGGAGTGGCTGGCACAC |
| | | | ATTTACTGGGATGATGACAAGCGCTATAACCCATCCCTG |
| | | | AAGAGCCGGCTCACAATCTCCAAGGATACCTCCAGAAAC |
| | | | CAGGTATTCCTCAAGATCACCAGTGTGGACACTGCAGAT |
| | | | ACTGCCACATACTACTGTGCTCGAAGAAGGAGGGGGTAT |
| | | | GGTATGGACTACTGGGGTCAAGGAACCTCAGTCACCGTC |
| | | | TCCTCA |
| | 1621 | LC CDR 1 | KASQSVSNDVA |
| | | (Chothia) | |
| | 1622 | LC CDR2 | YASNRCT |
| | | (Chothia) | |
| | 1623 | LC CDR3 | QQDYRSPLT |
| | | (Chothia) | |
| | 1621 | LC CDR1 | KASQSVSNDVA |
| | | (Kabat) | |
| | 1622 | LC CDR2 | YASNRCT |
| | | (Kabat) | |
| | 1623 | LC CDR3 | QQDYRSPLT |
| | | (Kabat) | |
| | 1624 | LC CDR1 | QSVSND |
| | | (IMGT) | |
| | 1625 | LC CDR2 | YAS |
| | | (IMGT) | |
| | 1623 | LC CDR3 | QQDYRSPLT |
| | | (IMGT) | |
| | 1626 | FR L1 | SIVMTQTPKFLLVSAGDRVTITC |
| | | (corresponding to the | |
| | | Chothia CDR | |
| | | definition) | |
| | 1545 | FR L2 | WYQQKPGQSPKLLIY |
| | | (corresponding to the | |
| | | Chothia CDR | |
| | | definition) | |
| | 1627 | FR L3 | GVPDRFTGSGYGTDFTFTISTVQAEDLAVYFC |
| | | (corresponding to the | |
| | | Chothia CDR | |
| | | definition) | |
| | 1628 | FR L4 | FGAGTKLELK |
| | | (corresponding to the | |
| | | Chothia CDR | |
| | | definition) | |
| | 1629 | VL | SIVMTQTPKFLLVSAGDRVTITCKASQSVSNDVAWYQQK |
| | | | PGQSPKLLIYYASNRCTGVPDRFTGSGYGTDFTFTISTV |
| | | | QAEDLAVYFCQQDYRSPLIFGAGTKLELK |
| | 1630 | DNA VL | AGTATTGTGATGACCCAGACTCCCAAATTCCTGCTTGTA |
| | | | TCAGCAGGAGACAGGGTTACCATAACCTGCAAGGCCAGT |
| | | | CAGAGTGTGAGTAATGATGTAGCTTGGTACCAACAGAAG |
| | | | CCAGGGCAGTCTCCTAAACTGCTAATATACTATGCATCC |
| | | | AATCGCTGCACTGGAGTCCCTGATCGCTTCACTGGCAGT |
| | | | GGATATGGGACGGATTTCACTTTCACCATCAGCACTGTA |
| | | | CAGGCTGAAGACCTGGCAGTTTATTTCTGTCAGCAGGAT |
| | | | TATAGGTCTCCGCTCACGTTCGGTGCTGGGACCAAGCTG |
| | | | GAGCTGAAA |

TABLE 14
Exemplary anti-tau antibodies

| Ab ID | SEQ ID | Description | Sequence |
|---|---|---|---|
| VY011 | 2200 | HCDR1 | GGSISNYY |
| | 2201 | HCDR2 | VYTSGSA |
| | 2202 | HCDR3 | ARDRGLYY |
| | 2203 | FR H1 | QVQLQESGPGLVKPSETLSLTCTVS |
| | 2204 | FR H2 | WTWIRQPAGKGLEWIGR |
| | 2205 | FR H3 | NYNPSLKSRVTMSVDTSKNQFSLTLSSVTAADTAVYYC |
| | 2206 | FR H4 | WGQGTLVTVSS |
| | 2207 | LCDR1 | QSLVHSDGNTY |
| | 2208 | LCDR2 | KIS |
| | 2209 | LCDR3 | MQATQFPLT |
| | 2210 | FR L1 | DIVMTQTPLSSLVILGQPASISCRSS |
| | 2211 | FR L2 | LSWLQQRPGQPPRLLIY |
| | 2212 | FR L3 | KRFFGVPDRFSGSGAGTDFTLKINRVEAEDVGIYYC |
| | 2213 | FR L4 | FGGGTKVEIK |
| | 2214 | VH | QVQLQESGPGLVKPSETLSLTCTVSGGSISNYYWTWIRQPA |
| | | | GKGLEWIGRVYTSGSANYNPSLKSRVTMSVDTSKNQFSLTL |
| | | | SSVTAADTAVYYCARDRGLYYWGQGTLVTVSS |
| | 2215 | VL | DIVMTQTPLSSLVTLGQPASISCRSSQSLVHSDGNTYLSWL |
| | | | QQRPGQPPRLLIYKISKRFFGVPDRFSGSGAGTDFTLKINR |
| | | | VEAEDVGIYYCMQATQFPLTFGGGTKVEIK |
| | 2216 | VH (DNA) | CAGGTGCAGCTGCAGGAGTCGGGCCCAGGACTGGTGAAGCC |
| | | | TTCGGAGACCCTGTCCCTCACCTGCACTGTCTCTGGTGGCT |
| | | | CCATCAGTAATTACTACTGGACCTGGATCCGGCAGCCCGCC |
| | | | GGGAAGGGACTGGAGTGGATTGGGCGTGTCTATACCAGTGG |
| | | | GAGCGCCAACTACAACCCCTCCCTCAAGAGTCGAGTTACCA |
| | | | TGTCAGTAGACACGTCCAAGAACCAGTTCTCCCTGACGCTG |
| | | | AGCTCTGTGACCGCCGCGGACACGGCCGTGTATTACTGTGC |
| | | | GAGAGATAGAGGACTCTACTACTGGGGCCAGGGAACCCTGG |
| | | | TCACCGTCTCCTCA |
| | 2217 | VL (DNA) | GATATTGTGATGACCCAGACTCCACTCTCCTCACTTGTCAC |
| | | | CCTTGGACAGCCGGCCTCCATCTCCTGCAGGTCTAGTCAAA |
| | | | GCCTCGTACACAGTGATGGAAACACCTATTTGAGTTGGCTT |
| | | | CAGCAGAGGCCAGGCCAGCCTCCAAGACTCCTAATTTATAA |
| | | | GATTTCTAAGCGGTTCTTTGGGGTCCCAGACAGATTCAGTG |
| | | | GCAGTGGGGCAGGGACAGATTTCACACTGAAAATCAACAGG |
| | | | GTGGAAGCTGAGGATGTCGGGATTTATTACTGCATGCAAGC |
| | | | TACACAATTTCCGCTCACTTTCGGCGGAGGGACCAAGGTGG |
| | | | AGATCAAA |
| VY007 | 2218 | HCDR1 | RFTFSNYN |
| | 2219 | HCDR2 | ISSSSSTI |
| | 2220 | HCDR3 | ASLGRGY |
| | 2221 | FR H1 | EVQLVESGGGLVQPGGSLRLSCAAS |
| | 2222 | FR H2 | MNWVRQAPGKGLEWISY |
| | 2223 | FR H3 | YYADSVKGRFTISRDNAKNSLSLQMNSLRDEDTAVYYC |
| | 2206 | FR H4 | WGQGTLVTVSS |
| | 2207 | LCDR1 | QSLVHSDGNTY |
| | 2208 | LCDR2 | KIS |
| | 2224 | LCDR3 | MQATQFPRT |
| | 2225 | FR L1 | DIVMTQTPLSSPVTLGQPASISCRSS |
| | 2211 | FR L2 | LSWLQQRPGQPPRLLIY |
| | 2226 | FR L3 | NRFSGVPDRFSGSGAGTDFTLKISRVEAEDVGVYYC |
| | 2227 | FR L4 | FGQGSKLEIK |
| | 2228 | VH | EVQLVESGGGLVQPGGSLRLSCAASRFTFSNYNMNWVRQAP |
| | | | GKGLEWISYISSSSSTIYYADSVKGRFTISRDNAKNSLSLQ |
| | | | MNSLRDEDTAVYYCASLGRGYWGQGTLVTVSS |
| | 2229 | VL | DIVMTQTPLSSPVTLGQPASISCRSSQSLVHSDGNTYLSWL |
| | | | QQRPGQPPRLLIYKISNRFSGVPDRFSGSGAGTDFTLKISR |
| | | | VEAEDVGVYYCMQATQFPRTFGQGSKLEIK |
| | 2230 | VH (DNA) | GAGGTGCAGCTGGTGGAGTCTGGGGGAGGCTTGGTACAGCC |
| | | | TGGGGGGTCCCTGAGACTCTCCTGTGCAGCCTCTAGATTCA |
| | | | CCTTCAGTAACTATAACATGAACTGGGTCCGCCAGGCTCCA |
| | | | GGGAAGGGGCTGGAGTGGATTTCATACATTAGTAGTAGTAG |
| | | | TAGTACCATATACTACGCAGACTCTGTGAAGGGCCGATTCA |
| | | | CCATCTCCAGAGACAATGCCAAGAACTCACTGTCTCTGCAA |
| | | | ATGAACAGCCTGAGAGACGAGGACACGGCTGTGTATTACTG |
| | | | TGCGAGTCTGGGGAGGGGCTACTGGGGCCAGGGAACCCTGG |
| | | | TCACCGTCTCCTCA |
| | 2231 | VL (DNA) | GATATTGTGATGACCCAGACTCCACTCTCCTCACCTGTCAC |
| | | | CCTTGGACAGCCGGCCTCCATCTCCTGCAGGTCTAGTCAAA |
| | | | GCCTCGTACACAGTGATGGAAACACCTACTTGAGTTGGCTT |
| | | | CAGCAGAGGCCAGGCCAGCCTCCAAGACTCCTAATTTATAA |
| | | | GATTTCTAACCGGTTCTCTGGGGTCCCAGACAGATTCAGTG |
| | | | GCAGTGGGGCAGGGACAGATTTCACACTGAAAATCAGCAGG |
| | | | GTGGAAGCTGAGGATGTCGGGGTTTATTATTGTATGCAAGC |
| | | | TACTCAATTTCCTCGCACTTTTGGCCAGGGGTCCAAGCTGG |
| | | | AGATCAAA |
| VY004 | 2232 | HCDR1 | GFTFSDYS |
| | 2233 | HCDR2 | IRSSSSII |
| | 2234 | HCDR3 | ARRGHFDY |
| | 2235 | FR H1 | EVQLVESGGGLVQAGGSLRLSCAAS |
| | 2236 | FR H2 | MNWVRQAPGKGLEWVSY |
| | 2237 | FR H3 | YYADSVKGRFTISRDNAKNSLHLQMNSLRDEDTAVYYC |
| | 2206 | FR H4 | WGQGTLVTVSS |
| | 2238 | LCDR1 | QSVSDY |
| | 2239 | LCDR2 | DVS |
| | 2240 | LCDR3 | QQRSNWPLT |
| | 2241 | FR L1 | EIVLIQSPATLSLSPGERAILSCRAS |
| | 2242 | FR L2 | LAWYQQKPGQAPRLFIY |
| | 2243 | FR L3 | KRATGIPARFSGSGSGTDFTLTISSLEPEDFAVYYC |
| | 2213 | FR L4 | FGGGTKVEIK |
| | 2244 | VH | EVQLVESGGGLVQAGGSLRLSCAASGFTFSDYSMNWVRQAP |
| | | | GKGLEWVSYIRSSSSIIYYADSVKGRFTISRDNAKNSLHLQ |
| | | | MNSLRDEDTAVYYCARRGHFDYWGQGTLVTVSS |
| | 2245 | VL | EIVLTQSPATLSLSPGERATLSCRASQSVSDYLAWYQQKPG |
| | | | QAPRLFIYDVSKRATGIPARFSGSGSGTDFTLTISSLEPED |
| | | | FAVYYCQQRSNWPLTFGGGTKVEIK |
| | 2246 | VH (DNA) | GAGGTGCAGCTGGTGGAGTCTGGGGGAGGCTTGGTACAGGC |
| | | | TGGGGGGTCCCTGAGACTCTCCTGTGCAGCCTCTGGATTCA |
| | | | CCTTCAGTGACTATAGCATGAACTGGGTCCGCCAGGCTCCA |
| | | | GGGAAGGGGCTGGAGTGGGTTTCATATATTAGGAGTAGTAG |
| | | | TAGTATCATATACTACGCAGACTCTGTGAAGGGCCGATTCA |
| | | | CCATCTCCAGAGACAATGCCAAGAACTCACTGCATTTGCAA |
| | | | ATGAACAGCCTGAGAGACGAGGACACGGCTGTGTATTACTG |
| | | | TGCGAGACGGGGTCACTTTGACTACTGGGGCCAGGGAACCC |
| | | | TGGTCACCGTCTCCTCA |
| | 2247 | VL (DNA) | GAAATTGTGTTGACACAGTCTCCAGCCACCCTGTCTTTGTC |
| | | | TCCAGGGGAAAGAGCCACCCTCTCCTGCAGGGCCAGTCAGA |
| | | | GTGTTAGCGACTACTTAGCCTGGTACCAACAGAAACCTGGC |
| | | | CAGGCTCCCAGGCTCTTCATCTATGATGTATCCAAGAGGGC |
| | | | CACTGGCATCCCAGCCAGGTTCAGTGGCAGTGGGTCTGGGA |
| | | | CAGACTTCACTCTCACCATCAGCAGCCTAGAGCCTGAAGAT |
| | | | TTTGCAGTTTATTACTGTCAGCAGCGTAGCAACTGGCCGCT |
| | | | CACTTTCGGCGGAGGGACCAAGGTGGAGATCAAA |
| VY006 | 2248 | HCDR1 | GFTFSSYT |
| | 2219 | HCDR2 | ISSSSSTI |
| | 2249 | HCDR3 | ASLGRGDC |
| | 2221 | FR H1 | EVQLVESGGGLVQPGGSLRLSCAAS |
| | 2236 | FR H2 | MNWVRQAPGKGLEWVSY |
| | 2250 | FR H3 | YYADSVKGRFTISRDNAKNSLYLQMNSLRDEDTAVYYC |
| | 2206 | FR H4 | WGQGTLVTVSS |
| | 2207 | LCDR1 | QSLVHSDGNTY |
| | 2208 | LCDR2 | KIS |
| | 2224 | LCDR3 | MQATQFPRT |
| | 2225 | FR L1 | DIVMTQTPLSSPVTLGQPASISCRSS |
| | 2211 | FR L2 | LSWLQQRPGQPPRLLIY |
| | 2251 | FR L3 | KRFSGVPDRFSGSGAGTDFTLKISRVEAEDVGVYYC |
| | 2252 | FR L4 | FGQGTRLEIK |
| | 2253 | VH | EVQLVESGGGLVQPGGSLRLSCAASGFTFSSYTMNWVRQAP |
| | | | GKGLEWVSYISSSSSTIYYADSVKGRFTISRDNAKNSLYLQ |
| | | | MNSLRDEDTAVYYCASLGRGDCWGQGTLVTVSS |
| | 2254 | VL | DIVMTQTPLSSPVTLGQPASISCRSSQSLVHSDGNTYLSWL |
| | | | QQRPGQPPRLLIYKISKRFSGVPDRFSGSGAGTDFTLKISR |
| | | | VEAEDVGVYYCMQATQFPRTFGQGTRLEIK |
| | 2255 | VH (DNA) | GAGGTGCAGCTGGTGGAGTCTGGGGGAGGCTTGGTACAGCC |
| | | | TGGGGGGTCCCTGAGACTCTCCTGTGCAGCCTCTGGATTCA |
| | | | CCTTCAGTAGTTATACCATGAACTGGGTCCGCCAGGCTCCA |
| | | | GGGAAGGGGCTGGAGTGGGTTTCATACATTAGTAGTAGTAG |
| | | | TAGTACCATATACTACGCAGACTCTGTGAAGGGCCGATTCA |
| | | | CCATCTCCAGAGACAATGCCAAGAACTCACTGTATCTGCAA |
| | | | ATGAACAGCCTGAGAGACGAGGACACGGCTGTGTATTACTG |
| | | | TGCGAGTCTGGGGAGAGGGGACTGCTGGGGCCAGGGAACCC |
| | | | TGGTCACCGTCTCCTCA |
| | 2256 | VL (DNA) | GATATTGTGATGACCCAGACTCCACTCTCCTCACCTGTCAC |
| | | | CCTTGGACAGCCGGCCTCCATCTCCTGCAGGTCTAGTCAAA |
| | | | GCCTCGTACACAGTGATGGAAACACCTACTTGAGTTGGCTT |
| | | | CAGCAGAGGCCAGGCCAGCCTCCAAGACTCCTAATTTATAA |
| | | | GATTTCTAAACGGTTCTCTGGGGTCCCAGACAGATTCAGTG |
| | | | GCAGTGGGGCAGGGACAGATTTCACACTGAAAATCAGCAGG |
| | | | GTGGAAGCTGAGGATGTCGGGGTTTATTACTGCATGCAAGC |
| | | | TACACAATTTCCTCGGACCTTCGGCCAAGGGACACGACTGG |
| | | | AGATTAAA |
| VY018 | 2200 | HCDR1 | GGSISNYY |
| | 2257 | HCDR2 | IYTSGDT |
| | 2258 | HCDR3 | ARAGIVGTPGLGMDV |
| | 2203 | FR H1 | QVQLQESGPGLVKPSETLSLTCTVS |
| | 2259 | FR H2 | WSWIRQPAGKGLEWIGR |
| | 2260 | FR H3 | YYNPSLQSRVTMSVDTSKNQFSLKLSAVTAADTAVYYC |
| | 2261 | FR H4 | WGQGTTVTVSS |
| | 2262 | LCDR1 | ALPKQY |
| | 2263 | LCDR2 | KDS |
| | 2264 | LCDR3 | QSADSSGTYRV |
| | 2265 | FR L1 | SYELTQPPSVSVSPGQTARITCSGE |
| | 2266 | FR L2 | AYWYQQKPGQAPVLVIY |
| | 2267 | FR L3 | ERPSGIPERFSGSSSGTTVILTISGVQAEDEADYYC |
| | 2268 | FR L4 | FGGGTKLTVL |
| | 2269 | VH | QVQLQESGPGLVKPSETLSLTCTVSGGSISNYYWSWIRQPA |
| | | | GKGLEWIGRIYTSGDTYYNPSLQSRVTMSVDISKNQFSLKL |
| | | | SAVTAADTAVYYCARAGIVGTPGLGMDVWGQGTTVTVSS |
| | 2270 | VL | SYELTQPPSVSVSPGQTARITCSGEALPKQYAYWYQQKPGQ |
| | | | APVLVIYKDSERPSGIPERFSGSSSGTTVILTISGVQAEDE |
| | | | ADYYCQSADSSGTYRVFGGGTKLTVL |
| | 2271 | VH (DNA) | CAGGTGCAGCTGCAGGAGTCGGGCCCAGGACTGGTGAAGCC |
| | | | TTCGGAGACCCTGTCCCTCACCTGCACTGTCTCTGGTGGCT |
| | | | CCATCAGTAATTACTACTGGAGTTGGATCCGGCAGCCCGCC |
| | | | GGGAAGGGTCTGGAGTGGATTGGGCGTATCTATACCAGTGG |
| | | | GGACACCTACTACAACCCCTCCCTCCAGAGTCGAGTCACCA |
| | | | TGTCAGTAGACACGTCCAAGAACCAGTTCTCCCTGAAGCTG |
| | | | AGCGCTGTGACCGCCGCGGACACGGCCGTCTATTACTGTGC |
| | | | GAGAGCGGGTATAGTGGGAACTCCGGGACTCGGTATGGACG |
| | | | TCTGGGGCCAAGGGACCACGGTCACCGTCTCCTCA |
| | 2272 | VL (DNA) | TCCTATGAGCTGACACAGCCACCCTCGGTGTCAGTGTCCCC |
| | | | AGGACAGACGGCCAGGATCACCTGCTCTGGAGAAGCATTGC |
| | | | CAAAGCAATATGCTTATTGGTACCAGCAGAAGCCAGGCCAG |
| | | | GCCCCTGTGCTGGTGATATATAAAGACAGTGAGAGGCCCTC |
| | | | AGGGATCCCTGAGCGATTCTCTGGCTCCAGCTCAGGGACAA |
| | | | CAGTCACGTTGACCATCAGTGGAGTCCAGGCAGAAGATGAG |
| | | | GCTGACTATTACTGTCAATCAGCAGACAGCAGTGGTACTTA |
| | | | TCGGGTGTTCGGCGGAGGGACCAAGCTGACCGTCCTA |
| VY003 | 2273 | HCDR1 | GFTFSSYR |
| | 2274 | HCDR2 | ISSSRSAI |
| | 2275 | HCDR3 | ATLGIGY |
| | 2276 | FR H1 | EVQMVESGGGLVQPGGSLTLSCAAS |
| | 2236 | FR H2 | MNWVRQAPGKGLEWVSY |
| | 2250 | FR H3 | YYADSVKGRFTISRDNAKNSLYLQMNSLRDEDTAVYYC |
| | 2206 | FR H4 | WGQGTLVTVSS |
| | 2207 | LCDR1 | QSLVHSDGNTY |
| | 2208 | LCDR2 | KIS |
| | 2277 | LCDR3 | MQVTQFPRT |
| | 2225 | FR L1 | DIVMTQTPLSSPVTLGQPASISCRSS |
| | 2211 | FR L2 | LSWLQQRPGQPPRLLIY |
| | 2278 | FR L3 | NRFSGVPDRFSGSGAGTDFTLKITRVEAEDVGIYYC |
| | 2279 | FR L4 | FGQGTKVEIR |
| | 2280 | VH | EVQMVESGGGLVQPGGSLTLSCAASGFTFSSYRMNWVRQAP |
| | | | GKGLEWVSYISSSRSAIYYADSVKGRFTISRDNAKNSLYLQ |
| | | | MNSLRDEDTAVYYCAILGIGYWGQGTLVTVSS |
| | 2281 | VL | DIVMTQTPLSSPVTLGQPASISCRSSQSLVHSDGNTYLSWL |
| | | | QQRPGQPPRLLIYKISNRFSGVPDRFSGSGAGTDFTLKITR |
| | | | VEAEDVGIYYCMQVTQFPRTFGQGTKVEIR |
| | 2282 | VH (DNA) | GAGGIGCAGATGGTGGAGTCTGGGGGAGGCTTGGTACAGCC |
| | | | TGGGGGGTCCCTGACACTCTCCTGTGCAGCCTCTGGATTCA |
| | | | CCTTCAGTAGCTATAGAATGAACTGGGTCCGCCAGGCTCCA |
| | | | GGGAAGGGGCTGGAGTGGGTTTCATACATTAGCAGTAGTAG |
| | | | AAGTGCCATATACTACGCAGACTCTGTGAAGGGCCGATTCA |
| | | | CCATCTCCAGAGACAATGCCAAGAACTCACTGTATCTGCAA |
| | | | ATGAACAGCCTGAGAGACGAGGACACGGCTGTGTATTACTG |
| | | | TGCGACCCTGGGGATAGGCTACTGGGGCCAGGGAACCCTGG |
| | | | TCACCGTCTCCTCA |
| | 2283 | VL (DNA) | GATATTGTGATGACCCAGACTCCACTCTCCTCACCTGTCAC |
| | | | CCTTGGACAGCCGGCCTCCATCTCCTGCAGGTCTAGTCAAA |
| | | | GCCTCGTACACAGTGATGGAAACACCTACTTGAGTTGGCTT |
| | | | CAGCAGAGGCCAGGCCAGCCTCCAAGACTCCTAATTTATAA |
| | | | GATTTCTAACCGGTTCTCTGGGGTCCCAGACAGATTCAGTG |
| | | | GCAGTGGGGCAGGGACAGATTTCACACTGAAAATCACCAGG |
| | | | GTGGAGGCTGAGGATGTCGGGATTTATTACTGCATGCAAGT |
| | | | TACACAATTTCCTCGGACGTTCGGCCAAGGGACCAAGGGG |
| | | | AAATCAGA |
| VY016 | 2284 | HCDR1 | GFTFSSYS |
| | 2285 | HCDR2 | ISRSGSTI |
| | 2286 | HCDR3 | ARGNWAY |
| | 2221 | FR H1 | EVQLVESGGGLVQPGGSLRLSCAAS |
| | 2236 | FR H2 | MNWVRQAPGKGLEWVSY |
| | 2287 | FR H3 | YYADSVEGRFTISRDNAYNSLYLQMISLRDDDTAVYYC |
| | 2288 | FR H4 | WGQGILVTVSS |
| | 2289 | LCDR1 | SSNIGAGYD |
| | 2290 | LCDR2 | GNS |
| | 2291 | LCDR3 | QSYDSSLSGSV |
| | 2292 | FR L1 | QSVLTQPPSVSGAPGQRVTISCTGS |
| | 2293 | FR L2 | VHWYQQLPGTAPKLLIY |
| | 2294 | FR L3 | NRPSGVPDRFSGSKSGTSASLAITGLQAEDETDFYC |
| | 2268 | FR L4 | FGGGTKLTVL |
| | 2295 | VH | EVQLVESGGGLVQPGGSLRLSCAASGFTFSSYSMNWVRQAP |
| | | | GKGLEWVSYISRSGSTIYYADSVEGRFTISRDNAYNSLYLQ |
| | | | MISLRDDDTAVYYCARGNWAYWGQGILVTVSS |
| | 2296 | VL | QSVLTQPPSVSGAPGQRVTISCTGSSSNIGAGYDVHWYQQL |
| | | | PGTAPKLLIYGNSNRPSGVPDRFSGSKSGTSASLAITGLQA |
| | | | EDETDFYCQSYDSSLSGSVFGGGTKLIVL |
| | 2297 | VH (DNA) | GAGGTGCAGCTGGTGGAGTCTGGGGGAGGCTTGGTACAGCC |
| | | | TGGGGGGTCCCTGAGACTCTCCTGTGCAGCCTCTGGATTCA |
| | | | CCTTCAGTAGCTATAGCATGAACTGGGTCCGCCAGGCTCCA |
| | | | GGGAAGGGGCTGGAGTGGGTTTCATACATTAGTCGTAGTGG |
| | | | TAGTACCATATACTACGCAGACTCTGTGGAGGGCCGATTCA |
| | | | CCATCTCCAGAGACAATGCCTACAACTCACTGTATCTGCAA |
| | | | ATGATCAGCCTGAGAGACGATGACACGGCTGTGTATTACTG |
| | | | TGCGAGAGGGAACTGGGCCTACTGGGGCCAGGGAATCCTGG |
| | | | TCACCGTCTCCTCA |
| | 2298 | VL (DNA) | CAGTCTGTGCTGACGCAGCCGCCCTCAGTGTCTGGGGCCCC |
| | | | AGGGCAGAGGGTCACCATCTCCTGCACTGGGAGCAGCTCCA |
| | | | ACATCGGGGCAGGTTATGATGTACACTGGTACCAGCAGCTT |
| | | | CCAGGAACAGCCCCCAAACTCCTCATCTATGGTAACAGCAA |
| | | | TCGGCCCTCAGGGGTCCCTGACCGATTCTCTGGCTCCAAGT |
| | | | CTGGCACCTCAGCCTCCCTGGCCATCACTGGGCTCCAGGCT |
| | | | GAGGATGAGACTGATTTTTACTGCCAGTCCTATGACAGCAG |
| | | | CCTGAGTGGTTCGGTTTTCGGCGGAGGGACCAAGCTGACCG |
| | | | TCCTG |
| VY017 | 2284 | HCDR1 | GFTFSSYS |
| | 2299 | HCDR2 | IRSSSSTI |
| | 2300 | HCDR3 | ARRSIADY |
| | 2221 | FR H1 | EVQLVESGGGLVQPGGSLRLSCAAS |
| | 2236 | FR H2 | MNWVRQAPGKGLEWVSY |
| | 2301 | FR H3 | YYADSVKGRFTISRDNAKKSLYLQMNSLRDEDTAVYYC |
| | 2206 | FR H4 | WGQGTLVTVSS |
| | 2302 | LCDR1 | QSVTSY |
| | 2303 | LCDR2 | DAS |
| | 2304 | LCDR3 | QQRSNWPYT |
| | 2305 | FR L1 | EIVLTQSPATLSSSPGERATLSCRAS |
| | 2306 | FR L2 | LNWYQQKPGQAPRLLIY |
| | 2307 | FR L3 | NRATGIPARFSGSGSGTDFTLTISSLEPEDFALYYC |
| | 2308 | FR L4 | FGQGTKLEIK |
| | 2309 | VH | EVQLVESGGGLVQPGGSLRLSCAASGFTFSSYSMNWVRQAP |
| | | | GKGLEWVSYIRSSSSTIYYADSVKGRFTISRDNAKKSLYLQ |
| | | | MNSLRDEDTAVYYCARRSIADYWGQGTLVTVSS |
| | 2310 | VL | EIVLTQSPATLSSSPGERATLSCRASQSVTSYLNWYQQKPG |
| | | | QAPRLLIYDASNRATGIPARFSGSGSGTDFTLTISSLEPED |
| | | | FALYYCQQRSNWPYTFGQGTKLEIK |
| | 2311 | VH (DNA) | GAGGTGCAGTTGGTGGAGTCTGGGGGAGGCTTGGTACAGCC |
| | | | TGGGGGGTCCCTGAGACTCTCCTGTGCAGCCTCTGGATTCA |
| | | | CCTTCAGTAGCTATAGCATGAACTGGGTCCGCCAGGCTCCA |
| | | | GGGAAGGGGCTGGAGTGGGTTTCATACATTAGGAGTAGTAG |
| | | | TAGTACCATATACTACGCAGACTCTGTGAAGGGCCGATTCA |
| | | | CCATCTCCAGAGACAATGCCAAGAAATCATTGTATCTGCAA |
| | | | ATGAATAGTCTGAGAGACGAGGACACGGCTGTGTATTACTG |
| | | | TGCGAGAAGGAGTATAGCTGACTACTGGGGCCAGGGAACCC |
| | | | TGGTCACCGTCTCCTCA |
| | 2312 | VL (DNA) | GAAATTGTGTTGACACAGTCTCCAGCCACCCTGTCTTCGTC |
| | | | TCCAGGGGAAAGAGCCACCCTCTCCTGCAGGGCCAGTCAGA |
| | | | GTGTTACCAGCTACTTAAACTGGTACCAACAGAAACCTGGC |
| | | | CAGGCTCCCAGGCTCCTCATCTATGATGCATCCAACAGGGC |
| | | | CACTGGCATCCCAGCCAGGTTCAGTGGCAGTGGGTCTGGGA |
| | | | CAGACTTCACTCTCACCATCAGCAGCCTCGAGCCTGAAGAT |
| | | | TTTGCACTTTATTACTGTCAGCAGCGTAGCAACTGGCCGTA |
| | | | CACTTTTGGCCAGGGGACCAAACTGGAGATCAAA |
| VY012 | 2313 | HCDR1 | GFTYSSYA |
| | 2314 | HCDR2 | ISGSSSIT |
| | 2315 | HCDR3 | AKGGRYGYFQH |
| | 2316 | FR H1 | EVQLLESGGGLVQPGGSLRLSCAAS |
| | 2317 | FR H2 | MSWVRRAPGKGLEWISI |
| | 2318 | FR H3 | YYADSMKGRFTISRDNSKNTLFLQMNSLRAEDTAVYYC |
| | 2206 | FR H4 | WGQGTLVTVSS |
| | 2319 | LCDR1 | NIGSKS |
| | 2320 | LCDR2 | DDS |
| | 2321 | LCDR3 | QVWDSSSDPVV |
| | 2322 | FR L1 | SYVLTQPPSVSVAPGQTARITCGGN |
| | 2323 | FR L2 | VHWYQQKPGQAPVLVVY |
| | 2324 | FR L3 | DRPSGIPERFSGSNSGNTATLTISRVEAGDEADYYC |
| | 2325 | FR L4 | FGGGTMLTVL |
| | 2326 | VH | EVQLLESGGGLVQPGGSLRLSCAASGFTYSSYAMSWVRRAP |
| | | | GKGLEWISIISGSSSITYYADSMKGRFTISRDNSKNTLFLQ |
| | | | MNSLRAEDTAVYYCAKGGRYGYFQHWGQGTLVTVSS |
| | 2327 | VI | SYVLTQPPSVSVAPGQTARITCGGNNIGSKSVHWYQQKPGQ |
| | | | APVLVVYDDSDRPSGIPERFSGSNSGNTATLTISRVEAGDE |
| | | | ADYYCQVWDSSSDPVVFGGGTMLTVL |
| | 2328 | VH (DNA) | GAGGTGCAGCTGTTGGAGTCTGGGGGAGGCTTGGTACAGCC |
| | | | GGGGGGGTCCCTGAGACTCTCCTGTGCAGCCTCGGGATTCA |
| | | | CCTATAGCAGCTATGCCATGAGCTGGGTCCGCCGGGCTCCA |
| | | | GGGAAGGGGCTGGAGTGGATCTCAATTATTAGTGGTAGTAG |
| | | | TAGTATCACATACTACGCAGACTCCATGAAGGGCCGGTTCA |
| | | | CTATCTCTAGAGACAATTCCAAGAACACGCTTTTTCTGCAA |
| | | | ATGAATAGCCTGAGAGCCGAGGACACGGCCGTTTATTACTG |
| | | | TGCGAAGGGGGGGAGGTACGGGTACTTCCAACACTGGGGCC |
| | | | AGGGCACCCTGGTCACCGTCTCCTCA |
| | 2329 | VL (DNA) | TCCTATGTGCTGACTCAGCCACCCTCGGTGTCAGTGGCCCC |
| | | | AGGACAGACGGCCAGGATTACCTGTGGGGGAAACAACATTG |
| | | | GAAGTAAAAGTGTGCACTGGTACCAGCAGAAGCCAGGCCAG |
| | | | GCCCCTGTGCTGGTCGTCTATGATGATAGCGACCGGCCCTC |
| | | | AGGGATCCCTGAGCGATTCTCTGGCTCCAACTCTGGGAACA |
| | | | CGGCCACCCTGACCATCAGCAGGGTCGAAGCCGGGGATGAG |
| | | | GCCGACTATTACTGTCAGGTGTGGGATAGTAGTAGTGATCC |
| | | | TGTGGTATTCGGCGGAGGGACCATGCTGACCGTCCTA |
| VY009 | 2330 | HCDR1 | GFSLSTSEVG |
| | 2331 | HCDR2 | IYWNDHK |
| | 2332 | HCDR3 | ARRVTGEGFDP |
| | 2333 | FR H1 | QITLKESGPTLVKPTQTLTLTCTLS |
| | 2334 | FR H2 | VGWIRQPPGKALEWLAI |
| | 2335 | FR H3 | RYSPSLKNRLIFTKDTSKNQVVLTMINMDPVDTATYYC |
| | 2206 | FR H4 | WGQGTLVTVSS |
| | 2262 | LCDR1 | ALPKQY |
| | 2336 | LCDR2 | KDT |
| | 2337 | LCDR3 | QSADSSGSYV |
| | 2338 | FR L1 | SYELTQPPSVSVSPGQTARITCSGD |
| | 2339 | FR L2 | AYWYQQKPGQAPVLVIF |
| | 2267 | FR L3 | ERPSGIPERFSGSSSGTTVILTISGVQAEDEADYYC |
| | 2340 | FR L4 | FGTGTKVTVL |
| | 2341 | VH | QITLKESGPTLVKPTQTLTLTCTLSGFSLSTSEVGVGWIRQ |
| | | | PPGKALEWLAIIYWNDHKRYSPSLKNRLTFTKDTSKNQVVL |
| | | | TMTNMDPVDTATYYCARRVTGEGFDPWGQGTLVTVSS |
| | 2342 | VL | SYELTQPPSVSVSPGQTARITCSGDALPKQYAYWYQQKPGQ |
| | | | APVLVIFKDTERPSGIPERFSGSSSGITVILTISGVQAEDE |
| | | | ADYYCQSADSSGSYVFGTGTKVTVL |
| | 2343 | VH (DNA) | CAGATCACCTTAAAGGAGTCTGGTCCTACGCTGGTGAAACC |
| | | | CACACAGACCCTCACGCTGACCTGCACCCTCTCTGGGTTCT |
| | | | CACTCAGCACTAGTGAAGTGGGTGTGGGCTGGATCCGTCAG |
| | | | CCCCCAGGAAAGGCCCTGGAGTGGCTTGCAATCATTTATTG |
| | | | GAATGATCATAAGCGCTACAGCCCATCTCTGAAGAACAGGC |
| | | | TCACCTTCACCAAGGACACCTCCAAAAACCAGGTGGTCCTT |
| | | | ACAATGACCAACATGGACCCTGTGGACACAGCCACATATTA |
| | | | TTGTGCACGCAGAGTAACTGGGGAGGGGTTCGACCCCTGGG |
| | | | GCCAGGGAACCCTGGTCACCGTCTCCTCA |
| | 2344 | VL (DNA) | TCCTATGAGCTGACACAGCCACCCTCGGTGTCAGTGTCCCC |
| | | | AGGACAGACGGCCAGGATCACCTGCTCTGGAGATGCATTGC |
| | | | CAAAGCAATATGCTTATTGGTACCAGCAGAAGCCAGGCCAG |
| | | | GCCCCTGTGCTGGTGATATTTAAAGACACTGAGAGGCCCTC |
| | | | AGGGATCCCTGAGCGATTCTCTGGCTCCAGCTCAGGGACAA |
| | | | CAGTCACGTTGACCATCAGTGGAGTCCAGGCAGAAGATGAG |
| | | | GCTGACTATTACTGTCAATCAGCAGACAGCAGTGGTTCTTA |
| | | | TGTCTTCGGAACTGGGACCAAGGTCACCGTCCTA |
| VY010 | 2345 | HCDR1 | GFTFSTYS |
| | 2346 | HCDR2 | ISSGSSYI |
| | 2347 | HCDR3 | TVTTHFHH |
| | 2348 | FR H1 | EVQLVESGGGLVKPGGSLRLSCAAS |
| | 2349 | FR H2 | MNWVRQAPGKGLEWVSS |
| | 2350 | FR H3 | YYADSVKGRFTISRDNAKNSLYLQMNSLRAEDTAVYYC |
| | 2206 | FR H4 | WGQGTLVTVSS |
| | 2351 | LCDR1 | QSLVYSDGNTY |
| | 2352 | LCDR2 | KVS |
| | 2353 | LCDR3 | MQGTHWPLT |
| | 2354 | FR L1 | DVVMTQSPLSLPVTLGQPASISCRSS |
| | 2355 | FR L2 | LNWFQQRPGQSPRRLIY |
| | 2356 | FR L3 | NRDSGVPDRFSGSGSGTDFTLKISRVEAEDVGVYYC |
| | 2252 | FR L4 | FGQGTRLEIK |
| | 2357 | VH | EVQLVESGGGLVKPGGSLRLSCAASGFTFSTYSMNWVRQAP |
| | | | GKGLEWVSSISSGSSYIYYADSVKGRFTISRDNAKNSLYLQ |
| | | | MNSLRAEDTAVYYCTVTTHFHHWGQGTLVTVSS |
| | 2358 | VL | DVVMTQSPLSLPVTLGQPASISCRSSQSLVYSDGNTYLNWF |
| | | | QQRPGQSPRRLIYKVSNRDSGVPDRFSGSGSGTDFTLKISR |
| | | | VEAEDVGVYYCMQGTHWPLTFGQGTRLEIK |
| | 2359 | VH (DNA) | GAGGTGCAGCTGGTGGAGTCTGGGGGAGGCCTGGTCAAGCC |
| | | | TGGGGGGTCCCTGAGACTCTCCTGTGCAGCCTCTGGATTCA |
| | | | CCTTCAGTACCTATAGCATGAACTGGGTCCGCCAGGCTCCA |
| | | | GGGAAGGGGCTGGAGTGGGTCTCATCCATTAGTAGTGGTAG |
| | | | TAGTTACATATACTACGCAGACTCAGTGAAGGGCCGATTCA |
| | | | CCATCTCCAGAGACAACGCCAAGAACTCACTGTATCTGCAA |
| | | | ATGAACAGCCTGAGAGCCGAGGACACGGCTGTGTATTACTG |
| | | | TACAGTAACTACACACTTCCACCACTGGGGCCAGGGCACCC |
| | | | TGGTCACCGTCTCCTCA |
| | 2360 | VL (DNA) | GATGTTGTGATGACTCAGTCTCCACTCTCCCTGCCCGTCAC |
| | | | CCTTGGACAGCCGGCCTCCATCTCCTGCAGGTCTAGTCAAA |
| | | | GCCTCGTATACAGTGATGGAAACACCTACTTGAATTGGTTT |
| | | | CAGCAGAGGCCAGGCCAATCTCCAAGGCGCCTAATTTATAA |
| | | | GGTTTCTAACCGGGACTCTGGGGTCCCAGACAGATTCAGCG |
| | | | GCAGTGGGTCAGGCACTGATTTCACACTAAAAATCAGCAGG |
| | | | GTGGAGGCTGAGGATGTTGGGGTTTATTACTGCATGCAAGG |
| | | | TACACACTGGCCTCTCACCTTCGGCCAAGGGACACGACTGG |
| | | | AGATTAAA |
| VY022 | 2345 | HCDR1 | GFTFSTYS |
| | 2346 | HCDR2 | ISSGSSYI |
| | 2347 | HCDR3 | TVTTHFHH |
| | 2348 | FR H1 | EVQLVESGGGLVKPGGSLRLSCAAS |
| | 2349 | FR H2 | MNWVRQAPGKGLEWVSS |
| | 2350 | FR H3 | YYADSVKGRFTISRDNAKNSLYLQMNSLRAEDTAVYYC |
| | 2206 | FR H4 | WGQGTLVTVSS |
| | 2361 | LCDR1 | SGINVGAYR |
| | 2362 | LCDR2 | YKSDSDK |
| | 2363 | LCDR3 | MIWHSSAWV |
| | 2364 | FR L1 | QAVLTQPSSLSASPGASARLTCTLR |
| | 2365 | FR L2 | LYWYQQKPGSPPQYLLR |
| | 2366 | FR L3 | QQGSGVPSRFSGSKDASANAGILLISGLQSEDEADYYC |
| | 2268 | FR L4 | FGGGTKLTVL |
| | 2367 | VH | EVQLVESGGGLVKPGGSLRLSCAASGFTFSTYSMNWVRQAP |
| | | | GKGLEWVSSISSGSSYIYYADSVKGRFTISRDNAKNSLYLQ |
| | | | MNSLRAEDTAVYYCTVTTHFHHWGQGTLVTVSS |
| | 2368 | VL | QAVLTQPSSLSASPGASARLICTLRSGINVGAYRLYWYQQK |
| | | | PGSPPQYLLRYKSDSDKQQGSGVPSRFSGSKDASANAGILL |
| | | | ISGLQSEDEADYYCMIWHSSAWVFGGGTKLTVL |
| | 2369 | VH (DNA) | GAGGTGCAGCTGGTGGAGTCTGGGGGAGGCCTGGTCAAGCC |
| | | | TGGGGGGTCCCTGAGACTCTCCTGTGCAGCCTCTGGATTCA |
| | | | CCTTCAGTACCTATAGCATGAACTGGGTCCGCCAGGCTCCA |
| | | | GGGAAGGGGCTGGAGTGGGTCTCATCCATTAGTAGTGGTAG |
| | | | TAGTTACATATACTACGCAGACTCAGTGAAGGGCCGATTCA |
| | | | CCATCTCCAGAGACAACGCCAAGAACTCACTGTATCTGCAA |
| | | | ATGAACAGCCTGAGAGCCGAGGACACGGCTGTGTATTACTG |
| | | | TACAGTAACTACACACTTCCACCACTGGGGCCAGGGCACCC |
| | | | TGGTCACCGTCTCCTCA |
| | 2370 | VL (DNA) | CAGGCTGTGCTGACTCAGCCGTCTTCCCTCTCTGCATCTCC |
| | | | TGGAGCATCAGCCAGACTCACCTGCACCTTGCGCAGTGGCA |
| | | | TCAATGTTGGTGCCTACAGGCTATACTGGTACCAGCAGAAG |
| | | | CCAGGAAGTCCTCCCCAGTATCTCCTGAGGTACAAATCAGA |
| | | | CTCAGATAAGCAGCAGGGCTCTGGAGTCCCCAGCCGCTTCT |
| | | | CTGGATCCAAAGATGCTTCGGCCAATGCAGGGATTTTACTC |
| | | | ATCTCTGGGCTCCAGTCTGAGGATGAGGCTGACTATTACTG |
| | | | TATGATTTGGCACAGCAGCGCTTGGGTGTTCGGCGGAGGGA |
| | | | CCAAGCTGACCGTCCTA |
| VY001 | 2371 | HCDR1 | GFTFSSYN |
| | 2372 | HCDR2 | ISSSSNTI |
| | 2373 | HCDR3 | ATLGRGY |
| | 2221 | FR H1 | EVQLVESGGGLVQPGGSLRLSCAAS |
| | 2222 | FR H2 | MNWVRQAPGKGLEWISY |
| | 2374 | FR H3 | YYADSVKGRFTVSRDNAKNSLYLQMNSLRDEDTAVYYC |
| | 2375 | FR H4 | WGQGTLVIASS |
| | 2207 | LCDR1 | QSLVHSDGNTY |
| | 2208 | LCDR2 | KIS |
| | 2224 | LCDR3 | MQATQFPRT |
| | 2376 | FR L1 | DVVMTQTPLSSPVTLGQPASISCRSS |
| | 2211 | FR L2 | LSWLQQRPGQPPRLLIY |
| | 2226 | FR L3 | NRFSGVPDRFSGSGAGTDFTLKISRVEAEDVGVYYC |
| | 2308 | FR L4 | FGQGTKLEIK |
| | 2377 | VH | EVQLVESGGGLVQPGGSLRLSCAASGFTFSSYNMNWVRQAP |
| | | | GKGLEWISYISSSSNTIYYADSVKGRFTVSRDNAKNSLYLQ |
| | | | MNSLRDEDTAVYYCATLGRGYWGQGTLVIASS |
| | 2378 | VL | DVVMTQTPLSSPVTLGQPASISCRSSQSLVHSDGNTYLSWL |
| | | | QQRPGQPPRLLIYKISNRFSGVPDRFSGSGAGTDFTLKISR |
| | | | VEAEDVGVYYCMQATQFPRTFGQGTKLEIK |
| | 2379 | VH (DNA) | GAGGTGCAACTGGTGGAGTCTGGGGGAGGCTTGGTACAGCC |
| | | | TGGGGGGTCCCTGAGACTCTCCTGTGCAGCCTCTGGATTCA |
| | | | CCTTCAGTAGCTATAACATGAACTGGGTTCGCCAGGCTCCA |
| | | | GGGAAGGGGCTGGAGTGGATTTCATACATTAGTAGTAGTAG |
| | | | TAATACCATATACTACGCAGACTCTGTGAAGGGCCGATTCA |
| | | | CCGTCTCCAGGGACAATGCCAAGAACTCACTGTATCTGCAA |
| | | | ATGAACAGCCTGAGAGACGAGGACACGGCTGTGTATTACTG |
| | | | TGCGACTCTGGGGAGGGGCTACTGGGGCCAGGGAACCCTGG |
| | | | TCATCGCCTCCTCA |
| | 2380 | VL (DNA) | GATGTTGTGATGACCCAGACTCCACTCTCCTCACCTGTCAC |
| | | | CCTTGGACAGCCGGCCTCCATCTCCTGCAGGTCTAGTCAAA |
| | | | GCCTCGTACACAGTGATGGAAACACCTACTTGAGTTGGCTT |
| | | | CAGCAGAGGCCAGGCCAGCCTCCAAGACTCCTAATTTATAA |
| | | | GATTTCTAACCGGTTCTCTGGGGTCCCAGACAGATTCAGTG |
| | | | GCAGTGGGGCAGGGACAGATTTCACACTGAAAATCAGCAGG |
| | | | GTGGAAGCTGAGGATGTCGGGGTTTATTACTGCATGCAAGC |
| | | | TACACAATTTCCTCGCACTTTTGGCCAGGGGACCAAGCTGG |
| | | | AGATCAAA |
| VY019 | 2381 | HCDR1 | GGSISSYY |
| | 2382 | HCDR2 | IDTSGST |
| | 2383 | HCDR3 | ARGGDGYRY |
| | 2203 | FR H1 | QVQLQESGPGLVKPSETLSLTCTVS |
| | 2204 | FR H2 | WTWIRQPAGKGLEWIGR |
| | 2384 | FR H3 | NYNPSLKSRVTMSIDTSKKQFSLKLSSVTAADTAVYYC |
| | 2206 | FR H4 | WGQGTLVTVSS |
| | 2262 | LCDR1 | ALPKQY |
| | 2263 | LCDR2 | KDS |
| | 2385 | LCDR3 | QSTDSSGSWV |
| | 2338 | FR L1 | SYELTQPPSVSVSPGQTARITCSGD |
| | 2386 | FR L2 | ACWYQQKPGQAPVLVIY |
| | 2387 | FR L3 | ERPSGIPERFSGSSSGTTVTLTITGVQAEDEADYYC |
| | 2268 | FR L4 | FGGGTKLTVL |
| | 2388 | VH | QVQLQESGPGLVKPSETLSLTCTVSGGSISSYYWTWIRQPA |
| | | | GKGLEWIGRIDTSGSTNYNPSLKSRVTMSIDTSKKQFSLKL |
| | | | SSVTAADTAVYYCARGGDGYRYWGQGTLVTVSS |
| | 2389 | VL | SYELTQPPSVSVSPGQTARITCSGDALPKQYACWYQQKPGQ |
| | | | APVLVIYKDSERPSGIPERFSGSSSGTTVILTITGVQAEDE |
| | | | ADYYCQSTDSSGSWVFGGGTKLTVL |
| | 2390 | VH (DNA) | CAGGTGCAGCTGCAGGAGTCGGGCCCAGGACTGGTGAAGCC |
| | | | TTCGGAGACCCTGTCCCTCACCTGCACTGTCTCTGGTGGCT |
| | | | CCATCAGTAGTTACTACTGGACCTGGATCCGGCAGCCCGCC |
| | | | GGGAAGGGACTGGAGTGGATTGGGCGTATCGATACCAGTGG |
| | | | GAGCACCAACTACAACCCCTCCCTCAAGAGTCGAGTCACCA |
| | | | TGTCAATAGACACGTCCAAGAAACAGTTCTCCCTGAAGCTG |
| | | | AGCTCTGTGACCGCCGCGGACACGGCCGTGTATTACTGTGC |
| | | | GAGAGGGGGAGATGGCTACCGCTACTGGGGCCAGGGAACCC |
| | | | TGGTCACCGTCTCCTCA |
| | 2391 | VL (DNA) | TCCTATGAGCTGACACAGCCACCCTCGGTGTCAGTGTCCCC |
| | | | AGGACAGACGGCCAGGATCACCTGCTCTGGAGATGCATTGC |
| | | | CAAAGCAATATGCTTGTTGGTACCAGCAGAAGCCAGGCCAG |
| | | | GCCCCTGTGCTGGTGATATATAAAGACAGTGAGAGGCCCTC |
| | | | AGGGATCCCTGAGCGATTCTCTGGCTCCAGCTCAGGGACAA |
| | | | CAGTCACGTTGACCATCACTGGAGTCCAGGCAGAAGATGAG |
| | | | GCTGACTATTACTGTCAATCCACAGACAGCAGTGGTTCTTG |
| | | | GGTGTTCGGCGGAGGGACCAAGCTGACCGTCCTA |
| VY020 | 2284 | HCDR1 | GFTFSSYS |
| | 2392 | HCDR2 | ISSSNSTI |
| | 2393 | HCDR3 | ARRSLGDY |
| | 2221 | FR H1 | EVQLVESGGGLVQPGGSLRLSCAAS |
| | 2222 | FR H2 | MNWVRQAPGKGLEWISY |
| | 2394 | FR H3 | KYADSVKGRFTISRDNAKNSLYLQMNSLRDEDTAVYYC |
| | 2395 | FR H4 | WGQGTLVIVSS |
| | 2396 | LCDR1 | QSVSSY |
| | 2239 | LCDR2 | DVS |
| | 2397 | LCDR3 | QQRRNWPYT |
| | 2241 | FR L1 | EIVLIQSPATLSLSPGERATLSCRAS |
| | 2398 | FR L2 | LAWYLQKPGQAPRLLIY |
| | 2399 | FR L3 | NRATGIPARFSGSGSGTDFTLTISRLEPEDFAVYYC |
| | 2308 | FR L4 | FGQGTKLEIK |
| | 2400 | VH | EVQLVESGGGLVQPGGSLRLSCAASGFTFSSYSMNWVRQAP |
| | | | GKGLEWISYISSSNSTIKYADSVKGRFTISRDNAKNSLYLQ |
| | | | MNSLRDEDTAVYYCARRSLGDYWGQGTLVIVSS |
| | 2401 | VL | EIVLTQSPATLSLSPGERATLSCRASQSVSSYLAWYLQKPG |
| | | | QAPRLLIYDVSNRATGIPARFSGSGSGTDFTLIISRLEPED |
| | | | FAVYYCQQRRNWPYTFGQGTKLEIK |
| | 2402 | VH (DNA) | GAGGTGCAGCTGGTGGAGTCTGGGGGAGGCTTGGTACAGCC |
| | | | TGGGGGGTCCCTGAGACTCTCCTGTGCAGCCTCTGGATTCA |
| | | | CCTTCAGTAGCTATAGCATGAACTGGGTCCGCCAGGCTCCA |
| | | | GGGAAGGGGCTGGAGTGGATTTCATACATTAGTAGTAGTAA |
| | | | TAGTACCATAAAGTACGCAGACTCTGTGAAGGGCCGATTCA |
| | | | CCATCTCCAGAGACAATGCCAAGAACTCACTGTATCTGCAA |
| | | | ATGAACAGCCTGAGAGACGAGGACACGGCTGTGTATTACTG |
| | | | TGCGAGAAGGAGTCTAGGTGACTACTGGGGCCAGGGAACCC |
| | | | TGGTCATCGTCTCCTCA |
| | 2403 | VL (DNA) | GAAATTGTGTTGACACAGTCTCCAGCCACCCTGTCTTTGTC |
| | | | TCCAGGGGAAAGAGCCACCCTCTCCTGCAGGGCCAGTCAGA |
| | | | GTGTTAGCAGCTACTTAGCCTGGTACCTACAGAAACCTGGC |
| | | | CAGGCTCCCAGGCTCCTCATCTATGATGTGTCCAACAGGGC |
| | | | CACTGGCATCCCAGCCAGGTTCAGTGGCAGTGGGTCTGGGA |
| | | | CAGACTTCACTCTCACCATCAGCAGACTAGAGCCTGAAGAT |
| | | | TTTGCAGTTTATTACTGTCAGCAGCGTCGCAACTGGCCGTA |
| | | | CACTTTTGGCCAGGGGACCAAGCTGGAGATCAAA |
| VY005 | 2284 | HCDR1 | GFTFSSYS |
| | 2392 | HCDR2 | ISSSNSTI |
| | 2393 | HCDR3 | ARRSLGDY |
| | 2221 | FR H1 | EVQLVESGGGLVQPGGSLRLSCAAS |
| | 2222 | FR H2 | MNWVRQAPGKGLEWISY |
| | 2394 | FR H3 | KYADSVKGRFTISRDNAKNSLYLQMNSLRDEDTAVYYC |
| | 2395 | FR H4 | WGQGTLVIVSS |
| | 2404 | LCDR1 | QGISCS |
| | 2405 | LCDR2 | AAS |
| | 2406 | LCDR3 | QQFNSYPFT |
| | 2407 | FR L1 | DIQLTQSPSFLSASVGDRVTITCRAS |
| | 2408 | FR L2 | LAWYQQKPGKAPKLLIY |
| | 2409 | FR L3 | TLQSGVPSRFSGSGSGTEFTLTISSLQPEDFATYYC |
| | 2410 | FR L4 | FGPGTKVDIK |
| | 2411 | VH | EVQLVESGGGLVQPGGSLRLSCAASGFTFSSYSMNWVRQAP |
| | | | GKGLEWISYISSSNSTIKYADSVKGRFTISRDNAKNSLYLQ |
| | | | MNSLRDEDTAVYYCARRSLGDYWGQGTLVIVSS |
| | 2412 | VL | DIQLTQSPSFLSASVGDRVTITCRASQGISCSLAWYQQKPG |
| | | | KAPKLLIYAASTLQSGVPSRFSGSGSGTEFTLTISSLQPED |
| | | | FATYYCQQFNSYPFTFGPGTKVDIK |
| | 2413 | VH (DNA) | GAGGTGCAGCTGGTGGAGTCTGGGGGAGGCTTGGTACAGCC |
| | | | TGGGGGGTCCCTGAGACTCTCCTGTGCAGCCTCTGGATTCA |
| | | | CCTTCAGTAGCTATAGCATGAACTGGGTCCGCCAGGCTCCA |
| | | | GGGAAGGGGCTGGAGTGGATTTCATACATTAGTAGTAGTAA |
| | | | TAGTACCATAAAGTACGCAGACTCTGTGAAGGGCCGATTCA |
| | | | CCATCTCCAGAGACAATGCCAAGAACTCACTGTATCTGCAA |
| | | | ATGAACAGCCTGAGAGACGAGGACACGGCTGTGTATTACTG |
| | | | TGCGAGAAGGAGTCTAGGTGACTACTGGGGCCAGGGAACCC |
| | | | TGGTCATCGTCTCCTCA |
| | 2414 | VL (DNA) | GACATCCAGTTGACCCAGTCTCCATCCTTCCTGTCTGCATC |
| | | | TGTAGGAGACAGAGTCACCATCACTTGCCGGGCCAGTCAGG |
| | | | GCATTAGCTGTTCTTTAGCCTGGTATCAGCAAAAACCAGGG |
| | | | AAAGCCCCTAAGCTCCTGATCTATGCTGCATCCACTTTGCA |
| | | | AAGTGGGGTCCCATCAAGGTTCAGCGGCAGTGGATCTGGGA |
| | | | CAGAATTCACTCTCACAATCAGCAGCCTGCAGCCTGAAGAT |
| | | | TTTGCAACTTATTACTGTCAACAGTTTAATAGTTACCCATT |
| | | | CACTTTCGGCCCTGGGACCAAAGTGGATATCAAA |
| VY002 | 2415 | HCDR1 | GFTFSNAW |
| | 2416 | HCDR2 | IKSKTDGGTT |
| | 2417 | HCDR3 | VTDYPKDV |
| | 2348 | FR H1 | EVQLVESGGGLVKPGGSLRLSCAAS |
| | 2418 | FR H2 | MSWVRQAPGKGLEWFGR |
| | 2419 | FR H3 | DYAAPVKGRFTISRDDSKNTLYLQMNSLKTEDTAVYYC |
| | 2261 | FR H4 | WGQGTTVTVSS |
| | 2420 | LCDR1 | QSVLYSSKNKNY |
| | 2421 | LCDR2 | WAS |
| | 2422 | LCDR3 | QQYYSSPYT |
| | 2423 | FR L1 | DIVMTQSPDSLAVSLGERATINCKSS |
| | 2424 | FR L2 | LAWYQQKPGQPPKLLIY |
| | 2425 | FR L3 | TRESGVPDRFSGSGSGTDFTLAISSLQAEDVAVYYC |
| | 2308 | FR L4 | FGQGTKLEIK |
| | 2426 | VH | EVQLVESGGGLVKPGGSLRLSCAASGFTFSNAWMSWVRQAP |
| | | | GKGLEWFGRIKSKTDGGTTDYAAPVKGRFTISRDDSKNTLY |
| | | | LQMNSLKTEDTAVYYCVTDYPKDVWGQGTTVTVSS |
| | 2427 | VL | DIVMTQSPDSLAVSLGERATINCKSSQSVLYSSKNKNYLAW |
| | | | YQQKPGQPPKLLIYWASTRESGVPDRFSGSGSGTDFTLAIS |
| | | | SLQAEDVAVYYCQQYYSSPYTFGQGTKLEIK |
| | 2428 | VH (DNA) | GAGGTGCAGCTGGTGGAGTCTGGGGGAGGCTTGGTAAAGCC |
| | | | TGGGGGGTCCCTTAGACTCTCCTGTGCAGCCTCTGGATTCA |
| | | | CTTTCAGTAACGCCTGGATGAGCTGGGTCCGCCAGGCTCCA |
| | | | GGGAAGGGGCTGGAGTGGTTTGGCCGTATTAAAAGCAAAAC |
| | | | TGATGGTGGGACAACAGACTACGCTGCACCCGTGAAAGGCA |
| | | | GATTCACCATCTCAAGAGATGACTCAAAAAACACGCTGTAT |
| | | | CTGCAAATGAACAGCCTGAAAACCGAGGACACAGCCGTGTA |
| | | | TTACTGTGTCACAGATTACCCGAAGGACGTCTGGGGCCAAG |
| | | | GGACCACGGTCACCGTCTCCTCA |
| | 2429 | VL (DNA) | GACATCGTGATGACCCAGTCTCCAGACTCCCTGGCTGTGTC |
| | | | TCTGGGCGAGAGGGCCACCATCAACTGCAAGTCCAGCCAGA |
| | | | GTGTTTTATACAGCTCCAAAAATAAGAACTACTTAGCTTGG |
| | | | TACCAGCAGAAACCAGGACAGCCTCCTAAGCTGCTCATTTA |
| | | | CTGGGCATCTACCCGGGAATCCGGGGTCCCTGACCGATTCA |
| | | | GTGGCAGCGGGTCTGGGACAGATTTCACTCTCGCCATCAGC |
| | | | AGCCTGCAGGCTGAAGATGTGGCAGTTTATTACTGTCAGCA |
| | | | ATATTATAGTAGTCCGTACACTTTTGGCCAGGGGACCAAGC |
| | | | TGGAGATCAAA |
| VY014 | 2430 | HCDR1 | GFTFNNYD |
| | 2431 | HCDR2 | IGAAGDT |
| | 2432 | HCDR3 | ARAGETLEGATIGYYYGMDV |
| | 2221 | FR H1 | EVQLVESGGGLVQPGGSLRLSCAAS |
| | 2433 | FR H2 | MHWVRQAAGKGLEWVST |
| | 2434 | FR H3 | YYPGSVKGRFIISRENAKNSLYLQMNSLRAGDTAVYYC |
| | 2261 | FR H4 | WGQGTTVTVSS |
| | 2435 | LCDR1 | QSLLHSNGYNY |
| | 2436 | LCDR2 | LGS |
| | 2437 | LCDR3 | MQALQIPLT |
| | 2438 | FR L1 | DILMTQSPLSLPVTPGEPASISCRSS |
| | 2439 | FR L2 | LDWYLQKPGQSPQLLIY |
| | 2440 | FR L3 | NRASGVPDRFSGSGSGTDFTLKFSRVEAEDVGLYYC |
| | 2213 | FR L4 | FGGGTKVEIK |
| | 2441 | VH | EVQLVESGGGLVQPGGSLRLSCAASGFTENNYDMHWVRQAA |
| | | | GKGLEWVSTIGAAGDTYYPGSVKGRFIISRENAKNSLYLQM |
| | | | NSLRAGDTAVYYCARAGETLEGATIGYYYGMDVWGQGTTVT |
| | | | VSS |
| | 2442 | VL | DILMTQSPLSLPVTPGEPASISCRSSQSLLHSNGYNYLDWY |
| | | | LQKPGQSPQLLIYLGSNRASGVPDRFSGSGSGTDFTLKFSR |
| | | | VEAEDVGLYYCMQALQIPLTFGGGTKVEIK |
| | 2443 | VH (DNA) | GAGGTGCAGCTGGTGGAGTCTGGGGGAGGCTTGGTACAGCC |
| | | | TGGGGGGTCCCTGAGACTCTCCTGTGCAGCCTCTGGATTCA |
| | | | CCTTCAATAACTACGACATGCACTGGGTCCGCCAAGCTGCA |
| | | | GGAAAAGGTCTGGAGTGGGTCTCAACTATTGGTGCTGCTGG |
| | | | TGACACATATTATCCAGGCTCCGTGAAGGGCCGATTCATCA |
| | | | TCTCCAGAGAAAATGCCAAGAACTCCTTGTATCTTCAAATG |
| | | | AACAGCCTGAGAGCCGGGGACACGGCTGTGTATTACTGTGC |
| | | | AAGAGCCGGAGAGACCTTAGAGGGAGCTACTATCGGCTACT |
| | | | ACTACGGTATGGACGTCTGGGGCCAGGGGACCACGGTCACC |
| | | | GTCTCCTCA |
| | 2444 | VL (DNA) | GATATTCTGATGACTCAGTCTCCACTCTCCCTGCCCGTCAC |
| | | | CCCTGGAGAGCCGGCCTCCATCTCCTGCAGGTCTAGTCAGA |
| | | | GCCTCCTGCATAGTAATGGATACAACTATTTGGATTGGTAC |
| | | | CTGCAGAAGCCAGGGCAGTCTCCACAGCTCCTGATCTATTT |
| | | | GGGTTCTAATCGGGCCTCCGGGGTCCCTGACAGGTTCAGTG |
| | | | GCAGTGGATCAGGCACAGATTTTACACTGAAATTCAGCAGA |
| | | | GTGGAGGCTGAGGATGTTGGACTTTATTACTGCATGCAAGC |
| | | | TCTACAAATTCCGCTCACTTTCGGCGGAGGGACCAAGGTGG |
| | | | AGATCAAA |
| VY008 | 2445 | HCDR1 | GYTFTGYY |
| | 2446 | HCDR2 | INPNSGGT |
| | 2447 | HCDR3 | AGDAFDI |
| | 2448 | FR H1 | QVQLVQSGAEVKKPGASVKVSCKAS |
| | 2449 | FR H2 | MHWVRQAPGQGLEWMGW |
| | 2450 | FR H3 | NYAQKFQGRVTMTRDISISTAYMELSRLRSDDTAVYYC |
| | 2451 | FR H4 | WGQGTMVTVSS |
| | 2452 | LCDR1 | QSVLYSSNNKNY |
| | 2421 | LCDR2 | WAS |
| | 2453 | LCDR3 | QQYYSTPLT |
| | 2423 | FR L1 | DIVMTQSPDSLAVSLGERATINCKSS |
| | 2424 | FR L2 | LAWYQQKPGQPPKLLIY |
| | 2454 | FR L3 | TRESGVPDRFSGSGSGTDFTLTISSLQAEDVAVYYC |
| | 2213 | FR L4 | FGGGTKVEIK |
| | 2455 | VH | QVQLVQSGAEVKKPGASVKVSCKASGYTFTGYYMHWVRQAP |
| | | | GQGLEWMGWINPNSGGTNYAQKFQGRVTMIRDTSISTAYME |
| | | | LSRLRSDDTAVYYCAGDAFDIWGQGTMVTVSS |
| | 2456 | VL | DIVMTQSPDSLAVSLGERATINCKSSQSVLYSSNNKNYLAW |
| | | | YQQKPGQPPKLLIYWASTRESGVPDRFSGSGSGIDFTLTIS |
| | | | SLQAEDVAVYYCQQYYSTPLTFGGGTKVEIK |
| | 2457 | VH (DNA) | CAGGTGCAGCTGGTGCAGTCTGGGGCTGAGGTGAAGAAGCC |
| | | | TGGGGCCTCAGTGAAGGTCTCCTGCAAGGCTTCTGGATACA |
| | | | CCTTCACCGGCTACTATATGCACTGGGTGCGACAGGCCCCT |
| | | | GGACAAGGGCTTGAGTGGATGGGATGGATCAACCCTAACAG |
| | | | TGGTGGCACAAACTATGCACAGAAGTTTCAGGGCAGGGTCA |
| | | | CCATGACCAGGGACACGTCCATCAGCACAGCCTACATGGAG |
| | | | CTGAGCAGGCTGAGATCTGACGACACGGCCGTGTATTACTG |
| | | | TGCTGGGGATGCTTTTGATATCTGGGGCCAAGGGACAATGG |
| | | | TCACCGTCTCTTCA |
| | 2458 | VL (DNA) | GACATCGTGATGACCCAGTCTCCAGACTCCCTGGCTGTGTC |
| | | | TCTGGGCGAGAGGGCCACCATCAACTGCAAGTCCAGCCAGA |
| | | | GTGTTTTATACAGCTCCAACAATAAGAACTACTTAGCTTGG |
| | | | TACCAGCAGAAACCAGGACAGCCTCCTAAGCTGCTCATTTA |
| | | | CTGGGCATCTACCCGGGAATCCGGGGTCCCTGACCGATTCA |
| | | | GTGGCAGCGGGTCTGGGACAGATTTCACTCTCACCATCAGC |
| | | | AGCCTGCAGGCTGAAGATGTGGCAGTTTATTACTGTCAGCA |
| | | | ATATTATAGTACTCCGCTCACTTTCGGCGGAGGGACCAAGG |
| | | | TGGAGATCAAA |
| VY013 | 2459 | HCDR1 | GFSLINARMA |
| | 2460 | HCDR2 | IFSNDEK |
| | 2461 | HCDR3 | ARIRGYSYNYGMDV |
| | 2462 | FR H1 | QVTLKESGPVLVKPTETLTLTCTVS |
| | 2463 | FR H2 | VSWIRQPPGKALEWLAH |
| | 2464 | FR H3 | SYSTSLKSRLTISKDTSKSQVVLIMINMDPVDTATYYC |
| | 2261 | FR H4 | WGQGTTVTVSS |
| | 2465 | LCDR1 | QSLLHSNGYIY |
| | 2466 | LCDR2 | VGS |
| | 2467 | LCDR3 | MQTLQIPRT |
| | 2468 | FR L1 | DIVMTQSPLSLPVIPGEPASISCRSS |
| | 2439 | FR L2 | LDWYLQKPGQSPQLLIY |
| | 2469 | FR L3 | NRASGVPDRFSGSGSGTDFKLKISRVEAEDVGVYYC |
| | 2213 | FR L4 | FGGGTKVEIK |
| | 2470 | VH | QVTLKESGPVLVKPTETLTLTCTVSGFSLINARMAVSWIRQ |
| | | | PPGKALEWLAHIFSNDEKSYSTSLKSRLTISKDTSKSQVVL |
| | | | IMTNMDPVDTATYYCARIRGYSYNYGMDVWGQGTTVTVSS |
| | 2471 | VL | DIVMTQSPLSLPVIPGEPASISCRSSQSLLHSNGYIYLDWY |
| | | | LQKPGQSPQLLIYVGSNRASGVPDRFSGSGSGTDFKLKISR |
| | | | VEAEDVGVYYCMQTLQIPRTFGGGTKVEIK |
| | 2472 | VH (DNA) | CAGGTCACCTTGAAGGAGTCTGGTCCTGTGCTGGTGAAACC |
| | | | CACAGAGACCCTCACGCTGACCTGCACCGTCTCTGGGTTCT |
| | | | CACTCATCAATGCTAGAATGGCTGTGAGCTGGATCCGTCAG |
| | | | CCCCCAGGGAAGGCCCTGGAGTGGCTTGCACACATTTTTTC |
| | | | GAATGACGAAAAATCCTACAGCACATCTCTGAAGAGCAGAC |
| | | | TCACCATCTCCAAGGACACCTCCAAAAGCCAGGTGGTCCTT |
| | | | ATCATGACCAACATGGACCCTGTGGACACAGCCACATATTA |
| | | | CTGTGCACGGATCCGTGGATACAGCTATAACTACGGTATGG |
| | | | ACGTCTGGGGCCAAGGGACCACGGTCACCGTCTCCTCA |
| | 2473 | VL (DNA) | GATATTGTGATGACTCAGTCTCCACTCTCCCTGCCCGTCAT |
| | | | CCCTGGAGAGCCGGCCTCCATCTCCTGCAGGTCTAGTCAGA |
| | | | GCCTCCTGCATAGTAATGGATACATCTATTTGGATTGGTAC |
| | | | CTGCAGAAGCCAGGGCAGTCTCCACAGCTCCTGATCTATGT |
| | | | GGGTTCTAATCGGGCCTCCGGGGTCCCTGACAGGTTCAGTG |
| | | | GCAGTGGATCAGGCACAGATTTCAAACTGAAAATCAGCAGA |
| | | | GTGGAGGCTGAGGATGTTGGGGTTTATTACTGCATGCAAAC |
| | | | TCTACAAATTCCGCGCACTTTCGGCGGAGGGACCAAGGTGG |
| | | | AGATCAAA |

TABLE 15
Variable domain CDR amino acid sequence sets

| | Variable | CDR1 SEQ | CDR2 SEQ | CDR3 SEQ |
|---|---|---|---|---|
| ID# | domain | ID NO | ID NO | ID NO |
| VY003 | VH | 2273 | 2274 | 2275 |
| VY016 | VH | 2284 | 2285 | 2286 |
| VY007 | VH | 2218 | 2219 | 2220 |
| VY004 | VH | 2232 | 2233 | 2234 |
| VY017 | VH | 2284 | 2299 | 2300 |
| VY006 | VH | 2248 | 2219 | 2249 |
| VY011 | VH | 2200 | 2201 | 2202 |
| VY012 | VH | 2313 | 2314 | 2315 |
| VY009 | VH | 2330 | 2331 | 2332 |
| VY010 | VH | 2345 | 2346 | 2347 |
| VY022 | VH | 2345 | 2346 | 2347 |
| VY018 | VH | 2200 | 2257 | 2258 |
| VY001 | VH | 2371 | 2372 | 2373 |
| VY019 | VH | 2381 | 2382 | 2383 |
| VY020 | VH | 2284 | 2392 | 2393 |
| VY005 | VH | 2284 | 2392 | 2393 |
| VY002 | VH | 2415 | 2416 | 2417 |
| VY014 | VH | 2430 | 2431 | 2432 |
| VY008 | VH | 2445 | 2446 | 2447 |
| VY013 | VH | 2459 | 2460 | 2461 |
| VY003 | VL | 2207 | 2208 | 2277 |
| VY016 | VL | 2289 | 2290 | 2291 |
| VY007 | VL | 2207 | 2208 | 2224 |
| VY004 | VL | 2238 | 2239 | 2240 |
| VY017 | VL | 2302 | 2303 | 2304 |
| VY006 | VL | 2207 | 2208 | 2224 |
| VY011 | VL | 2207 | 2208 | 2209 |
| VY012 | VL | 2319 | 2320 | 2321 |
| VY009 | VL | 2262 | 2336 | 2337 |
| VY010 | VL | 2351 | 2352 | 2353 |
| VY022 | VL | 2361 | 2362 | 2363 |
| VY018 | VL | 2262 | 2263 | 2264 |
| VY001 | VL | 2207 | 2208 | 2224 |
| VY019 | VL | 2262 | 2263 | 2385 |
| VY020 | VL | 2396 | 2239 | 2397 |
| VY005 | VL | 2404 | 2405 | 2406 |
| VY002 | VL | 2420 | 2421 | 2422 |
| VY014 | VL | 2435 | 2436 | 2437 |
| VY008 | VL | 2452 | 2421 | 2453 |
| VY013 | VL | 2465 | 2466 | 2467 |

TABLE 16
Variable domain CDR amino acid sequence set pairs

| | HCDR1 SEQ | HCDR2 SEQ | HCDR3 SEQ | LCDR1 | LCDR2 | LCDR3 |
|---|---|---|---|---|---|---|
| ID# | ID NO | ID NO | ID NO | SEQ ID NO | SEQ ID NO | SEQ ID NO |
| VY003 | 2273 | 2274 | 2275 | 2207 | 2208 | 2277 |
| VY016 | 2284 | 2285 | 2286 | 2289 | 2290 | 2291 |
| VY007 | 2218 | 2219 | 2220 | 2207 | 2208 | 2224 |
| VY004 | 2232 | 2233 | 2234 | 2238 | 2239 | 2240 |
| VY017 | 2284 | 2299 | 2300 | 2302 | 2303 | 2304 |
| VY006 | 2248 | 2219 | 2249 | 2207 | 2208 | 2224 |
| VY011 | 2200 | 2201 | 2202 | 2207 | 2208 | 2209 |
| VY012 | 2313 | 2314 | 2315 | 2319 | 2320 | 2321 |
| VY009 | 2330 | 2331 | 2332 | 2262 | 2336 | 2337 |
| VY010 | 2345 | 2346 | 2347 | 2351 | 2352 | 2353 |
| VY022 | 2345 | 2346 | 2347 | 2361 | 2362 | 2363 |
| VY018 | 2200 | 2257 | 2258 | 2262 | 2263 | 2264 |
| VY001 | 2371 | 2372 | 2373 | 2207 | 2208 | 2224 |
| VY019 | 2381 | 2382 | 2383 | 2262 | 2263 | 2385 |
| VY020 | 2284 | 2392 | 2393 | 2396 | 2239 | 2397 |
| VY005 | 2284 | 2392 | 2393 | 2404 | 2405 | 2406 |
| VY002 | 2415 | 2416 | 2417 | 2420 | 2421 | 2422 |
| VY014 | 2430 | 2431 | 2432 | 2435 | 2436 | 2437 |
| VY008 | 2445 | 2446 | 2447 | 2452 | 2421 | 2453 |
| VY013 | 2459 | 2460 | 2461 | 2465 | 2466 | 2467 |

TABLE 17
Constant Regions of Heavy Chains and Light Chains

| SEQ | | |
|---|---|---|
| ID | | |
| NO | Description | Sequence |
| 2474 | Constant | AKITPPSVYPLAPGSAAQTNSMVTLGCLVKGYFPEPVTVTWNSGSLSSGVHT |
| | Region of | FPAVLQSDLYTLSSSVTVPSSPRPSETVTCNVAHPASSTKVDKKIVPRDCGC |
| | Heavy | KPCICTVPEVSSVFIFPPKPKDVLTITLTPKVTCVVVDISKDDPEVQFSWFV |
| | Chain, | DDVEVHTAQTQPREEQFNSTFRSVSELPIMHQDWLNGKEFKCRVNSAAFPAP |
| | secreted | IEKTISKTKGRPKAPQVYTIPPPKEQMAKDKVSLICMITDFFPEDITVEWQW |
| | form | NGQPAENYKNTQPIMNINGSYFVYSKLNVQKSNWEAGNTFTCSVLHEGLHNH |
| | (mIgG1) | HTEKSLSHSPGK |
| 2475 | Constant | AKTTPPSVYPLAPGSAAQTNSMVTLGCLVKGYFPEPVTVTWNSGSLSSGVHT |
| | Region of | FPAVLQSDLYTLSSSVTVPSSPRPSETVTCNVAHPASSTKVDKKIVPRDCGC |
| | Heavy | KPCICTVPEVSSVFIFPPKPKDVLTITLTPKVTCVVVDISKDDPEVQFSWFV |
| | Chain, | DDVEVHTAQTQPREEQFNSTFRSVSELPIMHQDWLNGKEFKCRVNSAAFPAP |
| | membrane- | IEKTISKTKGRPKAPQVYTIPPPKEQMAKDKVSLTCMITDFFPEDITVEWQW |
| | bound form | NGQPAENYKNTQPIMNINGSYFVYSKLNVQKSNWEAGNTFTCSVLHEGLHNH |
| | (mIgG1) | HTEKSLSHSPGLQLDETCAEAQDGELDGLWTTITIFISLFLLSVCYSAAVTL |
| | | FKVKWIFSSVVELKQTLVPEYKNMIGQAP |
| 2476 | Constant | AKTTAPSVYPLAPVCGDTTGSSVTLGCLVKGYFPEPVTLTWNSGSLSSGVHT |
| | Region of | FPAVLQSDLYTLSSSVTVISSTWPSQSITCNVAHPASSTKVDKKIEPRGPTI |
| | murine | KPCPPCKCPAPNLLGGPSVFIFPPKIKDVLMISLSPIVTCVVVDVSEDDPDV |
| | Heavy | QISWFVNNVEVHTAQTQTHREDYNSTLRVVSALPIQHQDWMSGKEFKCKVNN |
| | Chain, | KDLPAPIERTISKPKGSVRAPQVYVLPPPEEEMTKKQVTLTCMVTDEMPEDI |
| | (mIgG2A) | YVEWINNGKTELNYKNTEPVLDSDGSYFMYSKLRVEKKNWVERNSYSCSVVH |
| | | EGLHNHHTTKSFSRTPGK |
| 2477 | DNA | GCTAAAACAACAGCCCCATCGGTCTATCCACTGGCCCCTGTGTGTGGAGATA |
| | Constant | CAACTGGCTCCTCGGTGACTCTAGGATGCCTGGTCAAGGGTTATTTCCCTGA |
| | Region of | GCCAGTGACCTTGACCTGGAACTCTGGATCCCTGTCCAGTGGTGTGCACACC |
| | Heavy | TTCCCAGCTGTCCTGCAGTCTGACCTCTACACCCTCAGCAGCTCAGTGACTG |
| | Chain | TAACCTCGAGCACCTGGCCCAGCCAGTCCATCACCTGCAATGTGGCCCACCC |
| | (mIgG2A) | GGCAAGCAGCACCAAGGTGGACAAGAAAATTGAGCCCAGAGGGCCCACAATC |
| | | AAGCCCTGTCCTCCATGCAAATGCCCAGCACCTAACCTCTTGGGTGGACCAT |
| | | CCGTCTTCATCTTCCCTCCAAAGATCAAGGATGTACTCATGATCTCCCTGAG |
| | | CCCCATAGTCACATGTGTGGTGGTGGATGTGAGCGAGGATGACCCAGATGTC |
| | | CAGATCAGCTGGTTTGTGAACAACGTGGAAGTACACACAGCTCAGACACAAA |
| | | CCCATAGAGAGGATTACAACAGTACTCTCCGGGTGGTCAGTGCCCTCCCCAT |
| | | CCAGCACCAGGACTGGATGAGTGGCAAGGAGTTCAAATGCAAGGTCAACAAC |
| | | AAAGACCTCCCAGCGCCCATCGAGAGAACCATCTCAAAACCCAAAGGGTCAG |
| | | TAAGAGCTCCACAGGTATATGTCTTGCCTCCACCAGAAGAAGAGATGACTAA |
| | | GAAACAGGTCACTCTGACCTGCATGGTCACAGACTTCATGCCTGAAGACATT |
| | | TACGTGGAGTGGACCAACAACGGGAAAACAGAGCTAAACTACAAGAACACTG |
| | | AACCAGTCCTGGACTCTGATGGTTCTTACTTCATGTACAGCAAGCTGAGAGT |
| | | GGAAAAGAAGAACTGGGTGGAAAGAAATAGCTACTCCTGTTCAGTGGTCCAC |
| | | GAGGGTCTGCACAATCACCACACGACTAAGAGCTTCTCCCGGACTCCGGGTA |
| | | AA |
| 2478 | Constant | AKTTAPSVYPLVPVCGGTTGSSVTLGCLVKGYFPEPVTLTWNSGSLSSGVHT |
| | Region of | FPALLQSGLYTLSSSVTVTSNTWPSQTITCNVAHPASSTKVDKKIEPRVPIT |
| | Heavy | QNPCPPHQRVPPCAAPDLLGGPSVFIFPPKIKDVLMISLSPMVTCVVVDVSE |
| | Chain, | DDPDVQISWFVNNVEVHTAQTQTHREDYNSTLRVVSALPIQHQDWMSGKEFK |
| | secreted | CKVNNRALPSPIEKTISKPRGPVRAPQVYVLPPPAEEMTKKEFSLICMITGF |
| | form | LPAEIAVDWTSNGRTEQNYKNTATVLDSDGSYFMYSKLRVQKSTWERGSLFA |
| | (mIgG2A) | CSVVHEVLHNHLTTKTISRSLGK |
| 2479 | Constant | KTIPPSVYPLAPGCGDTTGSSVTLGCLVKGYFPESVTVTWNSGSLSSSVHTF |
| | Region of | PALLQSGLYTMSSSVTVPSSTWPSQTVTCSVAHPASSTTVDKKLEPSGPIST |
| | murine | INPCPPCKECHKCPAPNLEGGPSVFIFPPNIKDVLMISLIPKVTCVVVDVSE |
| | Heavy | DDPDVQISWFVNNVEVHTAQTQTHREDYNSTIRVVSTLPIQHQDWMSGKEFK |
| | Chain, | CKVNNKDLPSPIERTISKIKGLVRAPQVYILPPPAEQLSRKDVSLTCLVVGF |
| | secreted | NPGDISVEWISNGHTEENYKDTAPVLDSDGSYFIYSKLNMKTSKWEKTDSFS |
| | form | CNVRHEGLKNYYLKKTISRSPGLDLDDICAEAKDGELDGLWTTITIFISLFL |
| | (mIgG2B) | LSVCYSASVTLFKVKWIFSSVVELKQKISPDYRNMIGQGA |
| 2480 | Constant | KITAPSVYPLAPVCGGTTGSSVTLGCLVKGYFPEPVILIWNSGSLSSGVHTF |
| | Region of | PALLQSGLYTLSSSVTVTSNTWPSQTITCNVAHPASSTKVDKKIEPRVPITQ |
| | murine | NPCPPLKECPPCAAPDLLGGPSVFIFPPKIKDVLMISLSPMVTCVVVDVSED |
| | Heavy | DPDVQISWFVNNVEVHTAQTQTHREDYNSTLRVVSALPIQHQDWMSGKEFKC |
| | Chain, | KVNNRALPSPIEKTISKPRGPVRAPQVYVLPPPAEEMIKKEFSLTCMITGFL |
| | (mIgG2B) | PAEIAVDWTSNGRTEQNYKNTATVLDSDGSYFMYSKLRVQKSTWERGSLFAC |
| | | SVVHEGLHNHLTTKTISRSLGLDLDDVCTEAQDGELDGLWITITIFISLFLL |
| | | SVCYSASVTLFKVKWIFSSVVELKQKISPDYRNMIGQGA |
| 2481 | Constant | TTTAPSVYPLVPGCSDTSGSSVTLGCLVKGYFPEPVTVKWNYGALSSGVRTV |
| | Region of | SSVLQSGFYSLSSLVTVPSSTWPSQTVICNVAHPASKTELIKRIEPRIPKPS |
| | murine | TPPGSSCPPGNILGGPSVFIFPPKPKDALMISLTPKVTCVVVDVSEDDPDVH |
| | Heavy | VSWFVDNKEVHTAWTQPREAQYNSTFRVVSALPIQHQDWMRGKEFKCKVNNK |
| | Chain, | ALPAPIERTISKPKGRAQTPQVYTIPPPREQMSKKKVSLTCLVTNFFSEAIS |
| | secreted | VEWERNGELEQDYKNTPPILDSDGTYFLYSKLTVDTDSWLQGEIFTCSVVHE |
| | form | ALHNHHTQKNLSRSPELELNETCAEAQDGELDGLWTTITIFISLFLLSVCYS |
| | (mIgG3) | ASVTLFKVKWIFSSVVQVKQTAIPDYRNMIGQGA |
| 2482 | Constant | ADAAPTVSIFPPSSEQLTSGGASVVCFLNNFYPKDINVKWKIDGSERQNGVL |
| | Region of | NSWTDQDSKDSTYSMSSTLTLTKDEYERHNSYTCEATHKTSTSPIVKSENRN |
| | Murine | EC |
| | Kappa Light | |
| | Chain | |
| 2483 | DNA | GCAGATGCTGCACCAACTGTATCCATCTTCCCACCATCCAGTGAGCAGTTAA |
| | Constant | CATCTGGAGGTGCCTCAGTCGTGTGCTTCTTGAACAACTTCTACCCCAAAGA |
| | Region of | CATCAATGTCAAGTGGAAGATTGATGGCAGTGAACGACAAAATGGCGTCCTG |
| | Murine | AACAGTTGGACTGATCAGGACAGCAAAGACAGCACCTACAGCATGAGCAGCA |
| | Kappa Light | CCCTCACGTTGACCAAGGACGAGTATGAACGACATAACAGCTATACCTGTGA |
| | Chain | GGCCACTCACAAGACATCAACTTCACCCATTGTCAAGAGCTTCAACAGGAAT |
| | | GAGTGT |
| 2484 | Constant | QPKSSPSVTLFPPSSEELEINKATLVCTITDFYPGVVTVDWKVDGTPVTQGM |
| | Region of | ETTQPSKQSNNKYMASSYLILTARAWERHSSYSCQVTHEGHTVEKSLSRADC |
| | murine | S |
| | Lambda | |
| | Light Chain | |
| | (subclass 1) | |
| 2485 | Constant | QPKSTPTLTVFPPSSEELKENKATLVCLISNFSPSGVTVAWKANGTPITQGV |
| | Region of | DTSNPTKEGNKFMASSFLHLTSDQWRSHNSFTCQVTHEGDTVEKSLSPAECL |
| | murine | |
| | Lambda | |
| | Light Chain | |
| | (subclass 2) | |
| 2486 | Constant | QPKSTPTLTMFPPSPEELQENKATLVCLISNFSPSGVTVAWKANGTPITQGV |
| | Region of | DTSNPTKEDNKYMASSFLHLTSDQWRSHNSFTCQVTHEGDTVEKSLSPAECL |
| | murine | |
| | Lambda | |
| | Light Chain | |
| | (subclass 3) | |
| 2487 | Constant | ASTKGPSVFPLAPSSKSTSGGTAALGCLVKDYFPEPVTVSWNSGALTSGVHT |
| | Region of | FPAVLQSSGLYSLSSVVTVPSSSLGTQTYICNVNHKPSNTKVDKRVEPKSCD |
| | human | KTHTCPPCPAPELLGGPSVFLFPPKPKDTLMISRTPEVTCVVVDVSHEDPEV |
| | Heavy | KFNWYVDGVEVHNAKTKPREEQYNSTYRVVSVLIVLHQDWLNGKEYKCKVSN |
| | Chain | KALPAPIEKTISKAKGQPREPQVYTLPPSREEMTKNQVSLTCLVKGFYPSDI |
| | (hIgG1) | AVEWESNGQPENNYKTTPPVLDSDGSFFLYSKLTVDKSRWQQGNVFSCSVMH |
| | | EALHNHYTQKSLSLSPG |
| 2488 | DNA | GCCAGCACAAAAGGGCCCAGTGTGTTCCCGCTCGCACCAAGCAGCAAATCAA |
| | Constant | CGTCAGGCGGCACAGCCGCGTTGGGTTGCCTTGTAAAAGACTACTTCCCAGA |
| | Region of | ACCAGTTACGGTGTCATGGAACAGTGGTGCACTCACGAGCGGCGTTCATACC |
| | human | TTCCCCGCCGTACTTCAGAGTTCAGGTCTTTACTCACTTTCCAGCGTGGTCA |
| | Heavy | CAGTACCGTCAAGCTCTCTTGGAACACAGACATATATCTGTAACGTAAATCA |
| | Chain | TAAGCCTAGCAATACCAAAGTCGATAAACGAGTGGAACCCAAGAGTTGTGAT |
| | (hIgG1) | AAAACCCACACCTGTCCCCCTTGCCCGGCACCGGAACTGCTGGGTGGTCCAT |
| | | CAGTATTCTTGTTTCCGCCTAAGCCAAAGGACACACTGATGATATCCAGAAC |
| | | TCCAGAGGTTACGTGCGTAGTCGTGGACGTCAGTCATGAAGACCCCGAAGTT |
| | | AAGTTCAACTGGTACGTGGATGGTGTGGAAGTACATAATGCGAAGACGAAAC |
| | | CCAGGGAAGAACAATATAACTCAACTTATAGGGTAGTCAGCGTCTTGACTGT |
| | | ACTTCACCAAGATTGGTTGAATGGCAAAGAGTACAAATGCAAGGTAAGCAAC |
| | | AAAGCATTGCCTGCGCCAATCGAAAAGACTATCTCAAAAGCAAAGGGCCAGC |
| | | CACGCGAACCACAAGTGTATACATTGCCGCCCAGTCGGGAAGAAATGACGAA |
| | | AAATCAAGTCAGTCTCACATGCCTCGTGAAAGGATTTTATCCCTCTGACATA |
| | | GCTGTGGAGTGGGAAAGTAATGGCCAACCCGAAAATAATTACAAAACGACGC |
| | | CTCCCGTTTTGGACTCAGATGGGAGTTTTTTCCTTTACAGTAAGCTGACGGT |
| | | TGACAAAAGCAGGTGGCAACAAGGGAACGTCTTTTCTTGTAGTGTGATGCAT |
| | | GAGGCGCTCCACAATCATTACACTCAAAAATCCTTGAGCCTGTCTCCAGGC |
| 2489 | Constant | ASTKGPSVFPLAPCSRSTSESTAALGCLVKDYFPEPVTVSWNSGALISGVHT |
| | Region of | FPAVLQSSGLYSLSSVVTVPSSNFGTQTYTCNVDHKPSNTKVDKTVERKCCV |
| | Heavy | ECPPCPAPPVAGPSVFLFPPKPKDTLMISRTPEVTCVVVDVSHEDPEVQFNW |
| | Chain | YVDGVEVHNAKTKPREEQFNSTFRVVSVLTVVHQDWLNGKEYKCKVSNKGLP |
| | (hIgG2) | APIEKTISKTKGQPREPQVYTLPPSREEMTKNQVSLTCLVKGFYPSDISVEW |
| | | ESNGQPENNYKTTPPMLDSDGSFFLYSKLTVDKSRWQQGNVFSCSVMHEALH |
| | | NHYTQKSLSLSPGK |
| 2490 | Constant | ASTKGPSVFPLAPCSRSTSGGTAALGCLVKDYFPEPVTVSWNSGALTSGVHT |
| | Region of | FPAVLQSSGLYSLSSVVTVPSSSLGTQTYTCNVNHKPSNTKVDKRVELKTPL |
| | Heavy | GDTTHTCPRCPEPKSCDTPPPCPRCPEPKSCDTPPPCPRCPEPKSCDTPPPC |
| | Chain | PRCPAPELLGGPSVFLFPPKPKDTLMISRTPEVTCVVVDVSHEDPEVQFKWY |
| | (hIgG3) | VDGVEVHNAKTKPREEQYNSTFRVVSVLTVLHQDWLNGKEYKCKVSNKALPA |
| | | PIEKTISKTKGQPREPQVYTLPPSREEMTKNQVSLTCLVKGFYPSDIAVEWE |
| | | SSGQPENNYNTTPPMLDSDGSFFLYSKLTVDKSRWQQGNIFSCSVMHEALHN |
| | | RFTQKSLSLSPGK |
| 2491 | Constant | ASTKGPSVFPLAPCSRSTSESTAALGCLVKDYFPEPVTVSWNSGALTSGVHT |
| | Region of | FPAVLQSSGLYSLSSVVTVPSSSLGTKTYTCNVDHKPSNTKVDKRVESKYGP |
| | human | PCPSCPAPEFLGGPSVFLFPPKPKDTLMISRTPEVTCVVVDVSQEDPEVQFN |
| | Heavy | WYVDGVEVHNAKTKPREEQFNSTYRVVSVLTVLHQDWLNGKEYKCKVSNKGL |
| | Chain | PSSIEKTISKAKGQPREPQVYTLPPSQEEMTKNQVSLTCLVKGFYPSDIAVE |
| | (hIgG4) | WESNGQPENNYKTTPPVLDSDGSFFLYSRLTVDKSRWQEGNVFSCSVMHEAL |
| | | HNHYTQKSLSLSLGK |
| 2492 | Constant | GQPKANPTVTLFPPSSEELQANKATLVCLISDFYPGAVTVAWKADGSPVKAG |
| | Region of | VETTKPSKQSNNKYAASSYLSLTPEQWKSHRSYSCQVTHEGSTVEKTVAPTE |
| | human | CS |
| | Light Chain | |
| | (hIgG | |
| | Lambda, | |
| | subclass 1) | |
| 2493 | Constant | GQPKAAPSVTLFPPSSEELQANKATLVCLISDFYPGAVTVAWKADSSPVKAG |
| | Region of | VETTTPSKQSNNKYAASSYLSLTPEQWKSHRSYSCQVTHEGSTVEKTVAPTE |
| | human | CS |
| | Light Chain | |
| | (hIgG | |
| | Lambda, | |
| | subclass 2) | |
| 2494 | Constant | GQPKAAPSVTLFPPSSEELQANKATLVCLISDFYPGAVTVAWKADSSPVKAG |
| | Region of | VETTTPSKQSNNKYAASSYLSLTPEQWKSHKSYSCQVTHEGSTVEKTVAPTE |
| | human | CS |
| | Light Chain | |
| | (hIgG | |
| | Lambda, | |
| | subclass 3) | |
| 2495 | Constant | GQPKAAPSVTLFPPSSEELQANKATLVCLISDFYPGAVKVAWKADGSPVNTG |
| | Region of | VETTTPSKQSNNKYAASSYLSLTPEQWKSHRSYSCQVTHEGSTVEKTVAPAE |
| | human | CS |
| | Light Chain | |
| | (hIgG | |
| | Lambda, | |
| | subclass 6) | |
| 2496 | Constant | GQPKAAPSVTLFPPSSEELQANKATLVCLVSDENPGAVTVAWKADGSPVKVG |
| | Region of | VETTKPSKQSNNKYAASSYLSLTPEQWKSHRSYSCRVTHEGSTVEKTVAPAE |
| | human | CS |
| | Light Chain | |
| | (hIgG | |
| | Lambda, | |
| | subclass 7) | |
| 2497 | Constant | TVAAPSVFIFPPSDEQLKSGTASVVCLLNNFYPREAKVQWKVDNALQSGNSQ |
| | Region of | ESVTEQDSKDSTYSLSSTLILSKADYEKHKVYACEVTHQGLSSPVTKSENRG |
| | human | EC |
| | Light Chain | |
| | (hIgG | |
| | Kappa) | |
| 2498 | DNA | ACTGTCGCAGCACCTTCTGTCTTCATCTTTCCGCCAAGCGATGAACAGTTGA |
| | Constant | AATCTGGAACAGCGTCCGTGGTGTGCCTGCTCAACAACTTCTATCCTCGGGA |
| | Region of | AGCGAAGGTGCAATGGAAGGTAGATAATGCTCTTCAGAGTGGCAATTCCCAA |
| | human | GAGTCAGTTACGGAGCAAGATAGCAAGGACAGCACGTATTCCCTGTCTAGTA |
| | Light Chain | CGTTGACTCTTTCCAAGGCTGACTATGAAAAGCACAAGGTGTATGCCTGTGA |
| | (hIgG | AGTAACCCACCAAGGTCTCTCAAGTCCTGTAACTAAGAGCTTTAATCGAGGA |
| | kappa) | GAATGC |

In some embodiments, an anti-tau antibody molecule may include CDRs identified through CDR analysis of variable domain sequences presented herein via co-crystallography with bound antigen; by computational assessments based on comparisons with other antibodies (e.g., see Strohl, W. R. Therapeutic Antibody Engineering. Woodhead Publishing, Philadelphia PA. 2012. Ch. 3, p47-54); or Kabat, Chothia, Al-Lazikani, Lefranc, or Honegger numbering schemes, as described previously.

In some embodiments, the AAV particles may comprise a viral genome, wherein one or more components may be codon-optimized. Codon-optimization may be achieved by any method known to one with skill in the art such as, but not limited to, by a method according to Genescript, EMBOSS, Bioinformatics, NUS, NUS2, Geneinfinity, IDT, NUS3, GregThatcher, Insilico, Molbio, N2P, Snapgene, and/or VectorNTI. Antibody heavy and/or light chain sequences within the same viral genome may be codon-optimized according to the same or according to different methods.

In some cases, such variants may include bispecific antibodies. Bispecific antibodies encoded by payload regions may comprise variable domain pairs from two different antibodies.

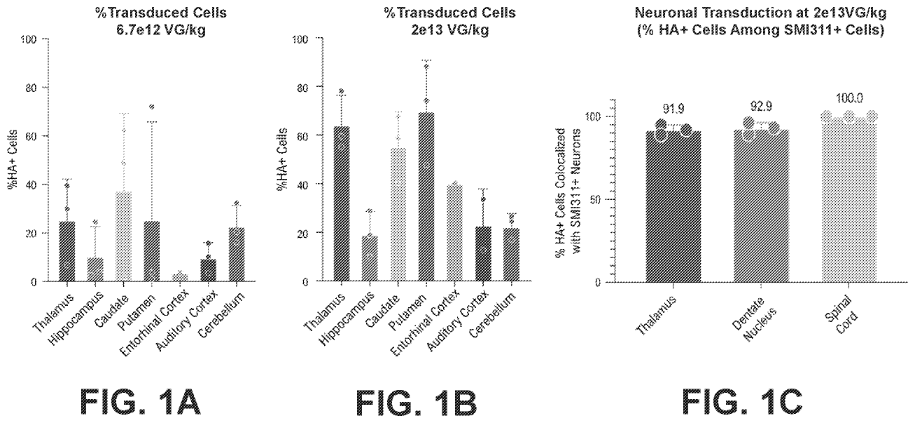

In some embodiments, the AAV particles may comprise a heavy and a light chain of an antibody described herein and two promoters. As a non-limiting example, the AAV particles may comprise a nucleic acid sequence of a genome as described in FIG. 1 or FIG. 2 of US Patent Publication No. US20030219733, the contents of which are herein incorporated by reference in their entirety. As another non-limiting example, the AAV particles may be a dual-promoter AAV for antibody expression as described by Lewis et al. (J. of. Virology, Sept 2002, Vol. 76(17), p 8769-8775; the contents of which are herein incorporated by reference in their entirety).

Payload regions of the viral genomes may encode any anti-tau antibody molecules, or tau-associated antibody molecules, not limited to those described in Tables 7-16, including antibodies that are known in the art and/or antibodies that are commercially available.

Anti-tau antibody molecules that may be encoded by payloads include, but are not limited to, AT8 (pSer²¹²/pThr²⁰⁵; ThermoFisher, Waltham, MA; described in International Publication No. WO1995017429, the contents of which are herein incorporated in their entirety), AT100 (pThr²¹²/pSer²¹⁴; ThermoFisher, Waltham, MA; described in U.S. Pat. No. 6,121,003, the contents of which are herein incorporated in their entirety), AT180 (pThr²³¹; ThermoFisher, Waltham, MA; described in International Publication No. WO1995017429, the contents of which are herein incorporated by reference in their entirety), MC-1 (or MC1) (Tau^{2-18/312-342} conformational antibody; as described in International Publication WO199620218, the contents of which are herein incorporated by reference in their entirety), MC-6 (pSer²³⁵; described in U.S. Pat. No. 5,811,310, the contents of which are herein incorporated in their entirety), TG-3 (pThr²³¹; described in Jicha, G A et al., 1997 J Neurochem 69(5):2087-95, the contents of which are herein incorporated by reference in their entirety), CP13 (pSer²⁰²), CP27 (human Tau¹³⁰⁻¹⁵⁰), Tau12 (human Tau⁹⁻¹⁸; Abcam, Cambridge, MA), TG5 (Tau²²⁰⁻²⁴²; described in U.S. Pat. No. 5,811,310), DA9 (Tau¹⁰²⁻¹⁴⁰; described in U.S. Pat. No. 5,811,310), PHF1 (or PHF-1) (pSer³⁹⁶/pSer⁴⁰⁴; described in International Publication WO199620218), Alz50 (Tau⁷⁻⁹ and Tau³¹²⁻³⁴² conformational epitope; described in U.S. Pat. No. 5,811,310 and Carmel, G et al 1996 J Biol Chem 271(51):32780-32795 and Jicha, G A et al, 1997 J Neurosci Res 48(2):128-132, the contents of each of which are herein incorporated by reference in their entirety), Tau-1 (de-phosphorylated Ser¹⁹⁵/Ser¹⁹⁸/Ser¹⁹⁹/Ser²⁰²; ThermoFisher, Waltham, MA), Tau46 (Tau⁴⁰⁴⁻⁴⁴¹; Abcam, Cambridge, MA), pS199 (ThermoFisher, Waltham, MA), pT205, pS396 (ThermoFisher, Waltham, MA; described in U.S. Pat. No. 8,647,631, the contents of which are herein incorporated by reference in their entirety), pS404 (ThermoFisher, Waltham, MA; described in U.S. Pat. No. 8,647,631, the contents of which are herein incorporated by reference in their entirety), pS422 (ThermoFisher, Waltham, MA), A0024 (hTau²⁴³⁻⁴⁴¹; Dako, Glostrup, Denmark), HT7 (hTau¹⁵⁹⁻¹⁶³; ThermoFisher, Waltham, MA), Tau2 (hTau⁵²⁻⁶⁸; Abcam, Cambridge, MA), AD2 (pSer³⁹⁶/pSer⁴° 4; Bio-Rad Laboratories, Hercules, CA), AT120 (hTau²¹⁶⁻²²⁴; described in U.S. Pat. No. 5,843,779, the contents of which are herein incorporated by reference in their entirety), AT270 (pThr¹⁸¹; ThermoFisher, Waltham, MA), 12E8 (pSer²⁶² and/or Ser³⁵⁶), K9JA (hTau²⁴³⁻⁴⁴¹; Dako, Caprinteria, CA), TauC3 (hTau Asp441; Santa Cruz Biotechnology, Dallas, TX; described in United States Patent Publication US20120244174 and Gamblin, T C et al 2003 PNAS 100(17):10032-7, the contents of each of which are herein incorporated by reference in their entirety), 4E6G7 (pSer³⁹⁶/pSer⁴⁰⁴; described in United States Patent Publication No. US2010316564 and Congdon, E. E. et al., 2016. Molecular Neurodegeneration Aug 30; 11(1):62, the contents of which are herein incorporated by reference in their entirety), 6B2 and variants thereof, described in International Patent Publication WO2016007414, the contents of which are herein incorporated by reference in their entirety, RZ3 (pThr²³¹), PG5 (pSer⁴⁰⁹), BT2 (pS^{199/202}), DA31 (Tau¹⁵⁰⁻¹⁹⁰), CP9 (pThr²³¹) Ta1505 (phospho site between Tau⁴¹⁰⁻⁴²¹, particularly pSer⁴¹³ as described in United States Patent Publication US20150183854 and Umeda, T. et al., 2015. Ann Clin Trans Neurol 2(3): 241-255, the contents of each of which are herein incorporated by reference in their entirety), PHF-6 (pThr²³¹, as described in Hoffman R et al., 1997. Biochemistry 36; 8114-8124, the contents of which are herein incorporated by reference in their entirety), PHF-13 (pSer³⁹⁶, as described in Hoffman R et al., 1997. Biochemistry 36; 8114-8124), 16B5 (Tau²⁵⁻⁴⁶, as described in United States Publication US20160031976, the contents of which are herein incorporated by reference in their entirety), DC8E8 (as described in United States Patent Publication US20150050215, the contents of which are herein incorporated by reference in their entirety), PT1 or PT3 (as described in U.S. Pat. No. 9,371,376, the contents of which are herein incorporated by reference in their entirety), 4G11 (Tau⁵⁷64, as described in International Publication WO2016137950, the contents of which are herein incorporated by reference in their entirety), 1A6 (Tau⁷⁻¹⁷ and Tau²¹⁵⁻²²⁰, as described in International Publication WO2016137950), Tau15 or Tau81 (as described in International Publication WO2016055941, the contents of which are herein incorporated by reference in their entirety), TOC-1 (dimerized or aggregated tau, as described in International Publication WO2012149365, the contents of which are herein incorporated by reference in their entirety), pS404IgG2a/k (Neotope Biosciences, South San Francisco, CA; as described in Ittner et al., 2015. Neurochemistry 132:135-145, the contents of which are herein incorporated by reference in their entirety), TOMA (tau oligomer monoclonal antibody; as described in United States Patent Nos. U.S. Pat. Nos. 8,778,343 and 9,125,846, International Publications WO2012051498 and WO2011026031, or United States Publication Nos. US20150004169 and US20150322143, and Castillo-Carranza, D L et al., 2014 J Neurosci 34(12)4260-72, the contents of each of which are herein incorporated by reference in their entirety), TTC-99 (oligomeric tau), BMS-986168 (as described in United States Patent Publication US2014294831, International Publication WO2015081085 and United States Patent U.S. Pat. No. 8,980,271, the contents of which are herein incorporated by reference in their entirety), 3H3 (pan-amyloid epitope; described in Levites, Y et al 2015 J Neurosci 35(16)6265-76, the contents of which are herein incorporated by reference in their entirety), cis-pT231 (described in International Publications WO2012149334 and WO2011056561, the contents of which are herein incorporated by reference in their entirety), CP-3 (pSer²¹⁴; described in Jicha et al 1999 J Neurosci 19(17):7486-94, the contents of which are herein incorporated by reference in their entirety), TNT1 (Tau²⁻¹⁸; as described in United States Patent Publication 20160031978, the contents of which are herein incorporated by reference in their entirety), Tau-nY29 (nTyr²⁹; described in Reynolds M R, et al., 2006 J Neurosci 26(42):10636-45, the contents of which are herein incorporated by reference in their entirety), Tau-nY197 (nTyr¹⁹⁷; described in Reyes, J F et al., 2012 Acta Neuropathol 123(1):119-32, the contents of which are herein incorporated by reference in their entirety), Tau-nY394 (nTyr³⁹⁴; described in Reyes, J F et al 2012), 4E4 (Tau³³⁷⁻³⁴³ Tau³⁸⁷⁻³⁹⁷; described in International Publication WO2012049570 and United States Patent Publication US20150252102, the contents of each of which are herein incorporated by reference in their entirety), ADx210 (described in United States Patent Publication US20140161875, the contents of which are herein incorporated by reference in their entirety), ADx215 (described in United States Patent Publication US20140161875), ADx202 (as described in International Publication WO2015004163, the contents of which are herein incorporated by reference in their entirety), AP422 (pSer⁴²²; described in Hasegawa, M et al 1996 FEBS Lett 384:25-30, the contents of which are herein incorporated by reference in their entirety), Tau5 (Tau²¹⁰⁻²⁴¹), RTA2 (Tau²⁷³⁻²⁸³), RTAC (Tau⁴²⁶⁻⁴⁴¹), RTA1 (Tau²⁵⁷⁻²⁷⁴), T46 (Tau³⁹⁵⁻⁴³²), T49, MIGT4, O.BG.15, 525, 3-39, 4F1, MapTau (Tau⁹⁵⁻¹⁰⁸; SMI Covance), T1, HYB33801 (Tau⁵⁻¹²), Tau13 (Tau²⁻¹⁸), B11E8, 5J20 (14-3-3 tau), DC25 (Tau³⁴⁷⁻³⁵³), DC39N1 (Tau⁴⁵⁻⁷³), DC-11 (Tau³²¹⁻³⁹¹; described in United States Patent U.S. Pat. No. 7,746,180, the contents of which are herein incorporated by reference in their entirety), DC39 (Tau⁴⁰¹⁻⁴¹¹), DC4R, n847 (nitrated tau), SPM452, TI4, 1E1/A6 (Tau²⁷⁵⁻²⁹¹), 5E2, 8E6/C11 (Tau²⁰⁹⁻²²⁴), 2E12 (pT231), NFT200, 248E5 (Tau³⁻²¹⁴), IG2 (Thr¹⁷⁵, Thr¹⁸¹, Thr²³¹; as described in International Publication WO2016041553, the contents of which are herein incorporated by reference in their entirety), YP3 (as described in WO2007019273, the contents of which are herein incorporated by reference in their entirety), YP4 (as described in WO2007019273), 14-3-3 Tau (pSer 14-3-3 binding motif; Abcam, Cambridge, MA), E2814, semorinemab (RG6100, R07105705, MTAU9937A), gosuranemab (BIIB092, BMS-986168, IPN007/IPN002), tilavonemab (ABBV-8E12, C2N-8E12, HJ8.5), zagotenemab (LY3303560, MC-1 IgG1), posdinemab (JNJ-63733657, B296, PT3), bepranemab (UCB0107), BIIB076 (N1-105.6C5 huIgG1λ), RG7345 (RO6926496), UCB0107 (D IgG4), NPT088, PNT001, Lu AF87908, PRX005, and APNmAb005. Further, anti-tau antibodies may be any of those listed in the antibody section of Alzforum.org or at the Antibody Resource Page.com, the contents of each of which are herein incorporated by reference in their entirety. Further, anti-tau antibodies may be any commercially available anti-tau antibody. Additional antibodies may include any of those taught in Petry, F. R. et al., 2014. PLoS One 9(5): e94251, the contents of which are herein incorporated by reference in their entirety. In one example, such antibodies may include any of those described in Jicha, G. A. et al., 1997. Journal of Neuroscience Research 48:128-132, the contents of which are herein incorporated by reference in their entirety. One such antibody, MC-1 (or MC1), recognizes distinct conformations of tau that are associated with neurological disease. In some embodiments, anti-tau antibodies may be any of those described in Plotkin et al. (Neurobiology of Disease 2020; 144:105010).

In some embodiments, the AAV particles may have a payload region comprising any of the anti-tau antibodies as described in International Publication WO2017189963, the contents of which are herein incorporated by reference in their entirety. As a non-limiting example, the payload region may comprise one or more of the anti-tau antibodies as described in Table 3 of International Publication WO2017189963. In some embodiments, the payload region encodes one or more anti-tau antibodies selected from SEQ ID NO: 2948-4269 as described in WO2017189963.

In some embodiments, payloads may encode anti-tau antibodies (or fragments thereof) taught in United States Publication No. US2014294831, the contents of which are herein incorporated by reference in their entirety. Such antibodies may include IPN001 and/or IPN002 antibodies or fragments of such antibodies. In some cases, variable domains of IPN002 as presented in FIGS. 2A and 2B of US2014294831 may be used (e.g., incorporated into another antibody). In some cases, CDR regions of IPN002 as underlined in FIGS. 2A and 2B may be used (e.g., incorporated into another antibody or used to prepare humanized versions of IPN002). In some cases, anti-tau antibodies may include any of the IPN001 or IPN002 antibody variants taught in US2014294831 (e.g., in FIGS. 9-16 of that publication). In some embodiments, this antibody is also referred to as BMS-986168.

In some cases, payloads may encode anti-tau antibodies (or fragments thereof) taught in Otvos, L. et al., 1994. J Neurosci. Res 39(6):669-73, the contents of which are herein incorporated by reference in their entirety. Such antibodies may include monoclonal antibody PHF-1 or fragments thereof. The PHF-1 antibody binds to tau paired helical filaments, a pathological conformation of tau, found in certain neurological disorders, including Alzheimer's disease. Further, antibody affinity is increased when either serine 396 or serine 404 of tau is phosphorylated and even further increased when both are phosphorylated.

In some embodiments, payloads may encode anti-tau antibodies (or fragments thereof) taught in U.S. Pat. No. 5,811,310, the contents of which are herein incorporated by reference in their entirety. Such embodiments may include monoclonal antibodies PHF-1 or MC1 or fragments thereof. MC1 is a conformational antibody binding to the epitopes presented in Jicha, G. A., et al., 1997. J Neurosci Res 48(128-132).

In some embodiments, payloads may encode anti-tau antibodies (or fragments thereof) taught in International Publication Number WO2015035190, the contents of which are herein incorporated by reference in their entirety. Such embodiments may include, but are not limited to, antibodies PHF-1 or MC1 or fragments thereof. Viral genomes of the AAV particles of the present disclosure may comprise or encode any of SEQ ID NO: 1-6 of WO2015035190.

In some embodiments, viral genomes may encode anti-tau antibody MC1 scFv as described in Vitale et al 2018, (Acta Neuropath Commun. 6:82), the contents of which are herein incorporated by reference in their entirety.

In some embodiments, viral genomes may encode anti-tau antibody MC1 as described in International Publication WO2016137811, the contents of which are herein incorporated by reference in their entirety.

Anti-tau antibody molecules encoded by viral genomes may include antibodies that bind to one or more of the epitopes presented in Otvos, L. et al., 1994. J Neurosci. Res 39(6):669-73 (e.g., any of those presented in Table 1 of that publication).

In some embodiments, payloads may encode anti-tau antibody molecules taught in U.S. Pat. No. 7,746,180, the contents of which are herein incorporated by reference in their entirety. Such embodiments may include antibody DC-11 or fragments thereof.

In some embodiments, the antibody molecules encoded by the viral genomes of the present disclosure may target any of the antigenic regions or epitopes described in United States Patent Publication No US2008050383 or US20100316564, the contents of which are herein incorporated by reference in their entirety. In some embodiments, the antibody targets pS396/pS404. Such embodiments may include antibody 4E6 and/or variants or fragments thereof. The affinity of antibody 4E6 for soluble PHF and its ability to reduce soluble phospho tau has been described in Congdon, E. E. et al., 2016. Molecular Neurodegeneration Aug 30; 11(1):62, the contents of which are herein incorporated by reference in their entirety.

In some embodiments, the antibody molecules encoded by the viral genomes of the present disclosure may target any of the antigenic regions or epitopes described in International Patent Publication WO1998022120, the contents of which are herein incorporated by reference in their entirety. In some embodiments, the antibody may be PHF-6 (pT231), or fragments or variants thereof. In another embodiment, the antibody may be PHF-13 (pS396), or a fragment of variant thereof. These antibodies are further described in Hoffman et al., 1997. Biochemistry 36: 8114-8124, the contents of which are herein incorporated by reference in their entirety.

In some embodiments, the antibodies encoded by the viral genomes of the present disclosure may target any of the antigenic regions or epitopes described in International Publication WO2016126993, the contents of which are herein incorporated by reference in their entirety. The antibodies may be derived from any of the tau epitopes described in Table A of WO2016126993. In some embodiments, the antibody of the present disclosure may comprise any of the sequences listed in Table B or Table 1 of WO2016126993.

In some embodiments, the antibody molecules encoded by the viral genomes of the present disclosure may target any of the antigenic regions or epitopes described in United States Patent Publication US20120244174, the contents of which are herein incorporated by reference in their entirety. In some embodiments, the antibody may bind to caspase-cleaved tau. In some embodiments, the epitope for antibodies targeting caspase cleaved tau is aspartic acid 421. In another embodiment, the epitope for antibodies targeting caspase cleaved tau may be the C-terminus after glutamic residue Glu391. In yet another embodiment, the epitope for antibodies targeting caspase cleaved tau may be at the N-terminus at aspartic acid residue 13. In another embodiment, the antibody may be TauC3.

In some embodiments, the antibody molecules encoded by the viral genomes of the present disclosure may target any of the antigenic regions or epitopes described in United States Patent Publication US20160031978, the contents of which are herein incorporated by reference in their entirety. In some embodiments, the antibody may bind to tau N-terminal residues associated with the PP1/GSK3 signaling cascade. In some embodiments, the antibody may be TNT1.

In some embodiments, the antibody molecules encoded by the viral genomes of the present disclosure may be any of those described in d'Abramo, C et al., 2015. PLOS One 10(8):e0135774, the contents of which are herein incorporated by reference in their entirety. In some embodiments, the antibody may be CP13 (pS202), or a fragment or variant thereof. In another embodiment, the antibody may be RZ3 (pT231), or a fragment or variant thereof. In another embodiment, the antibody may be PG5 (pS409), or a fragment or variant thereof.

In some embodiments, the antibody molecules encoded by the viral genomes of the present disclosure may be any of those described in WO2018/106781, WO2015/200806, WO2014/165271, WO2017/191559, WO2014/028777, WO2015/197820, WO2019110571, WO2017/191561, WO2015/120364, WO2015/122922, WO2004/016655, WO2014/152157, WO2015/197823, WO2018/170351, WO2019/171258, WO2019/171259, WO2021/205359, WO2017/005732, WO2019/094595, WO2022/144406, WO2012/149365, WO2017/005734, U.S. Pat. No. 7,176,290, WO2013/041962, WO2016/079597, WO2020/097561, WO2020/180819, WO2015/197820, WO2018/178077, WO2019/077500, US2007/0280935, WO2010/142423, U.S. Pat. Nos. 8,609,097, 9,290,567, WO2015/091656, U.S. Pat. Nos. 9,562,091, 10,465,000, WO2015/114538, U.S. Pat. Nos. 10,087,245, 10,538,582, 11,124,563, WO2015/197735, U.S. Pat. No. 10,251,952, EP3164152B1, WO2016/207245, U.S. Pat. Nos. 9,862,763, 10,822,402, EP3313877B1, WO2012/045882, U.S. Pat. Nos. 9,304,138, 10,100,104, EP2625198, WO2013/050567, U.S. Pat. Nos. 9,540,434, 10,066,010, EP2764022B9, EP3135689B1, WO2013/151762, U.S. Pat. No. 9,657,091, EP2834270B1, WO2012/106363, U.S. Pat. No. 8,703,137, U.S. Ser. No. 10/894,822, EP2670434B1, WO2013/180238, EP2857039B1, EP3662931, US2015/0183854, WO2014/096321, WO2014/150877, U.S. Pat. No. 9,598,485, WO2017/009308, U.S. Ser. No. 10/196,439, U.S. Ser. No. 10/562,962, U.S. Ser. No. 10/934,348, US2021/0206843, EP3322442, WO2018/127519, U.S. Ser. No. 10/995,137, EP3565836, WO2017/191560, U.S. Ser. No. 10/899,638, US2021/0130449, EP3452508, WO2020/201828, U.S. Ser. No. 11/155,609, EP3946605A1, WO2021/262791, US2021/0403541, WO2018/154390, U.S. Ser. No. 10/556,950, U.S. Ser. No. 10/894,829, US2021/0380677, EP3585810, WO2016/196726, WO2014/100600, the contents of all of which are herein incorporated by reference in their entirety.

In some embodiments, the antibody molecules encoded by the viral genomes of the present disclosure bind to tau protein antigens. Tau protein antigens may include human microtubule-associated protein tau, isoform 2 (SEQ ID NO: 9200) or fragments thereof. Tau protein antigens may include ePHF or fragments thereof. Tau protein antigens may include one or more phosphorylated residues. Such phosphorylated residues may correspond to those found with pathological tau. In some embodiments tau protein antigens include any of those listed in Table 18. In the Table, phosphorylated residues associated with each antigen are denoted as (pS) for a phosphorylated serine and (pT) for phosphorylated threonine. In some embodiments, tau proteins may include variants (e.g., phosphorylated or unphosphorylated variants) or fragments of the sequences listed.

TABLE 18
Exemplary tau protein antigen sequences

| | | SEQ |
|---|---|---|
| | | ID |
| Antigen | Sequence | NO |
| human microtubule- | MAEPRQEFEVMEDHAGTYGLGDRKDQGGYTMHQDQEGDTDAGLKESPLQ | 9200 |
| associated | TPTEDGSEEPGSETSDAKSTPTAEDVTAPLVDEGAPGKQAAAQPHTEIP | |
| protein tau, | EGTTAEEAGIGDTPSLEDEAAGHVTQARMVSKSKDGIGSDDKKAKGADG | |
| isoform 2 | KTKIATPRGAAPPGQKGQANATRIPAKTPPAPKTPPSSGEPPKSGDRSG | |
| | YSSPGSPGTPGSRSRTPSLPTPPTREPKKVAVVRTPPKSPSSAKSRLQT | |
| | APVPMPDLKNVKSKIGSTENLKHQPGGGKVQIINKKLDLSNVQSKCGSK | |
| | DNIKHVPGGGSVQIVYKPVDLSKVTSKCGSLGNIHHKPGGGQVEVKSEK | |
| | LDFKDRVQSKIGSLDNITHVPGGGNKKIETHKLIFRENAKAKTDHGAEI | |
| | VYKSPVVSGDISPRHLSNVSSTGSIDMVDSPQLATLADEVSASLAKQGL | |
| PT3 epitope peptide | TPGSRSR(pT)PSLP(pT)PPTREPK | 2137 |
| (pT212/pT217) | | |
| Peptide 5 (tPeptide 5) | GTPGSRSR(pT)P(pS)LP(pT)PPTRE | 2138 |
| (pT212/pS214/pT217) | | |
| Peptide 12 | RENAKAKTDHGAEIVYK(pS)PVVSGDT(pS)PRHL(pS)NVSSTG | 2139 |
| (pS396/pS404/pS409) | | |
| Peptide 1 (AT120 | PTREPKKV | 2140 |
| epitope) | | |
| 6C5 epitope peptide | ARMVSKS | 2141 |
| UCB D & PT76 | SPSSAKSRLQTAPVPMPDLKNVKS | 2142 |
| epitope peptide | | |
| Tau(pS404) | DHGAEIVYKSPVVSGDT(pS)PRHLSNVSSTG | 2143 |
| AC04 peptide | CSR(pT)PSLP(pT)PPTREPK | 2144 |
| PepScan 193-210 | DR(pS)GY(pS)(pS)PG(pS)PG(pT)PG(pS)R(pS) | 2145 |
| peptide | | |
| Tau 191-214 phospho- | SGDRSGYS(pS)PGSPGTPGSRSRTPS | 2146 |
| peptide (pS199) | | |
| Tau191-214 phospho- | SGDRSGYSSPG(pS)PGTPGSRSRTPS | 2147 |
| peptide (pS202) | | |
| Tau191-214 phospho- | SGDRSGYSSPGSPG(pT)PGSRSRTPS | 2148 |
| peptide (pT205) | | |
| Tau191-214 phospho- | SGDRSGYS(pS)PG(pS)PG(pT)PGSRSRTPS | 2149 |
| peptide | | |
| (pS199/pS202/pT205) | | |
| Tau191-214 phospho- | SGDRSGYS(pS)PG(pS)PGTPGSRSRTPS | 2150 |
| peptide (pS199/pS202) | | |
| Tau 191-214 phospho- | SGDRSGYSSPG(pS)PG(pT)PGSRSRTPS | 2151 |
| peptide (pS202/pT205) | | |
| Tau191-214 phospho- | SGDRSGYS(pS)PGSPG(pT)PGSRSRTPS | 2152 |
| peptide (pS199/pT205) | | |
| Tau204-222 phospho- | GTPGSRSR(pT)PSLPTPPTRE | 2153 |
| peptide (pT212) | | |
| Tau204-222 phospho- | GTPGSRSRTP(pS)LPTPPTRE | 2154 |
| peptide (pS214) | | |
| Tau204-222 phospho- | GTPGSRSRTPSLP(pT)PPTRE | 2155 |
| peptide (pT217) | | |
| Tau204-222 phospho- | GTPGSRSR(pT)P(pS)LPTPPTRE | 2156 |
| peptide (pT212/pS214) | | |
| Tau204-222 phospho- | GTPGSRSR(pT)PSLP(pT)PPTRE | 2157 |
| peptide (pT212/pT217) | | |
| Tau204-222 phospho- | GTPGSRSRTP(pS)LP(pT)PPTRE | 2158 |
| peptide (pS214/pT217) | | |
| Tau204-222 phospho- | GTPGSRSR(pT)P(pS)LP(pT)PPTRE | 2159 |
| peptide | | |
| (pT212/pS214/pT217) | | |
| Tau217-234 phospho- | (pT)PP(pT)REPKKVAVVR(pT)PPK | 2160 |
| peptide | | |
| (pT217/pT220/pT231) | | |
| Tau225-240 phospho- | KVAVVR(pT)PPKSPSSAK | 2161 |
| peptide (pT231) | | |
| Tau phospho-peptide | KVAVVRTPPK(pS)PSSAKPS | 2162 |
| (pS235) | | |
| Tau phospho-peptide | KVAVVR(pT)PPK(pS)PSSAKPS | 2163 |
| (pT231/pS235) | | |

In some embodiments, the antibody molecules encoded by the viral genomes of the present disclosure bind to tau protein epitopes on tau protein antigens described herein. Such tau protein epitopes may include or be included within a tau protein antigen amino acid sequence listed in Table 18. In some embodiments, anti-tau antibodies of the present disclosure bind to tau protein epitopes that include a region formed by a complex of at least two tau proteins.

In some embodiments, the antibody molecules encoded by the viral genomes of the present disclosure exhibit binding that overlaps with a region of tau recognized by art-recognized antibodies, such as AT8 and PT3, but exhibit binding patterns to phosphorylated tau that differs from the art-recognized antibodies.

In some embodiments, the antibody molecules encoded by the viral genomes of the present disclosure binds to human tau, wherein the antibody binds to all or a portion of amino acid residues of tau selected from: (a) 183-212, (b) 187-218, (c) 33-82, 159-182, 197-226, and 229-246; (d) 217-242, (e) 35-76 and 187-218, (f) 5-34, (g) 187-218, (h) 33-82, 159-188, and 191-230, (i) 35-62, 107-124, and 203-220, (j) 35-82, 159-188, and 197-224, or (k) 53-78, 329-348, and 381-408, wherein human tau is numbered according to SEQ ID NO: 9200. In some embodiments, one or more of the serines, threonines, and/or tyrosines in the stretch of amino acids selected from (a)-(k) are phosphorylated. In some embodiments, all of the serines, threonines, and/or tyrosines in the stretch of amino acids selected from (a)-(k) are phosphorylated. In some embodiments, the antibody comprises the HCDR1, HCDR2, HCDR3, LCDR1, LCDR2, and LCDR3 sequences, or the VH and VL sequences, of an antibody listed in Table 14-16.

In some embodiments, the antibody molecules encoded by the viral genomes of the present disclosure bind to all or a portion of amino acids 195-215 of human tau (e.g., phosphorylated at all serines, threonines, and/or tyrosines present in this stretch of amino acids) with a dissociation constant (K_{D}) of about 1 pM to about 50 pM, or about 1-25 pM, e.g., as assessed by bio-layer interferometry.

In some embodiments, the antibody molecules encoded by the viral genomes of the present disclosure bind to all or a portion of amino acids 191-214 of human tau phosphorylated at S199 (e.g., phosphorylated only at S199 in this stretch of amino acids or throughout the entire tau protein), e.g., with a dissociation constant (K_{D}) of about 0.1 nM to about 10 nM, or about 0.5-5 nM, e.g., as assessed by bio-layer interferometry.

In some embodiments, the antibody molecules encoded by the viral genomes of the present disclosure bind to all or a portion of amino acids 217-234 of human tau phosphorylated at T217, T220, and T231 (e.g., phosphorylated only at T217, T220, and T231 in this stretch of amino acids or throughout the entire tau protein), e.g., with a dissociation constant (K_{D}) of about 0.1 nM to about 10 nM, or about 0.1-5 nM, e.g., as assessed by bio-layer interferometry.

In some embodiments, the antibody molecules encoded by the viral genomes of the present disclosure bind to all or a portion of amino acids 225-240 of tau phosphorylated at T231 (e.g., phosphorylated only at T231 in this stretch of amino acids or throughout the entire tau protein), e.g., with a dissociation constant (K_{D}) of about 0.1 nM to about 25 nM, or about 0.1-15 nM, e.g., as assessed by bio-layer interferometry.

In some embodiments, the antibody molecules encoded by the viral genomes of the present disclosure bind to human tau phosphorylated at amino acid residue 5404, or a peptide comprising or consisting of the amino acid sequence DHGAEIVYKSPVVSGDT(pS)PRHLSNVSSTG (SEQ ID NO: 2143), wherein p(S) corresponds to a phosphorylated serine residue. In some embodiments, the antibody comprises a heavy chain variable region (VH) comprising CDR1, CDR2, and CDR3 sequences comprising SEQ ID NOs: 2430, 2431, and 2432, respectively, and a light chain variable region (VL) comprising CDR1, CDR2, and CDR3 sequences comprising SEQ ID NOs: 2435, 2436, and 2437, respectively. In some embodiments, the antibody comprises VH and VL sequences comprising SEQ ID NOs: 2441 and 2442, respectively.

In some embodiments, the antibody molecules encoded by the viral genomes of the present disclosure bind to: (a) human tau phosphorylated at amino acid residue S199, but not at amino acid residues S202 and T205; (b) human tau phosphorylated at amino acid residue S202, but not at amino acid residues S199 and T205; (c) human tau phosphorylated at amino acid residue T205, but not at amino acid residues S199 and S202; (d) human tau phosphorylated at a combination of amino acid residues S199 and T205, but not at amino acid residue S202 (e.g., wherein binding tau phosphorylated at a combination of S199 and T205 is at least 3-times stronger (e.g., at least 4-time stronger) than background (e.g., non-specific) level of binding, e.g., binding by hIgG1 isotype control); (e) human tau phosphorylated at a combination of amino acid residues S202 and T205, but not at amino acid residue S199, but not human tau phosphorylated at a combination of residues S199 and S202, but not T205; (f) human tau phosphorylated at a combination of amino acid residues: (i) S202 and T205, but not S119, and (ii) S199 and T205, but not S202, e.g., at least 2 times (e.g., at least 3 times, at least 4 times, at least 5 times, 2-6 times, 2-5 times, 2-4 times, 2-3 times, 3-5 times or 4-5 times) more strongly than background (e.g., non-specific) level of binding, e.g., binding by hIgG1 isotype control); (g) human tau phosphorylated at a combination of amino acid residues: (i) 5199 and 5202, but not T205, (ii) 5202 and T205, but not S199, (iii) S199 and T205, but not 5202, and (iv) S199, S202, and T205 (e.g., wherein binding to phosphorylated tau is at least 1.6-times stronger (e.g., at least 1.7 times, at least 1.8 times, at least 1.9 times, at least 2 times, at least 3 times, 1.6-3 times, 1.6-2 times stronger) than background (e.g., non-specific) level of binding, e.g., binding by hIgG1 isotype control); (h) a peptide comprising or consisting of the amino acid sequence SGDRSGYS(pS)PGSPGTPGSRSRTPS (SEQ ID NO: 2146); (i) a peptide comprising or consisting of the amino acid sequence SGDRSGYSSPG(pS)PGTPGSRSRTPS (SEQ ID NO: 2147); (j) a peptide comprising or consisting of the amino acid sequence SGDRSGYSSPGSPG(pT)PGSRSRTPS (SEQ ID NO: 2148); (k) a peptide comprising or consisting of the amino acid sequence SGDRSGYS(pS)PGSPG(pT)PGSRSRTPS (SEQ ID NO: 2152) (e.g., wherein binding to the peptide is at least 3 times stronger (e.g., at least 4 times stronger) than background (e.g., non-specific) level of binding, e.g., binding by hIgG1 isotype control); (1) a peptide comprising or consisting of the amino acid sequence SGDRSGYSSPG(pS)PG(pT)PGSRSRTPS (SEQ ID NO: 2151), but not a peptide comprising or consisting of the amino acid sequence SGDRSGYS(pS)PG(pS)PGTPGSRSRTPS (SEQ ID NO: 2150); (m) peptides comprising or consisting of the amino acid sequences SGDRSGYSSPG(pS)PG(pT)PGSRSRTPS (SEQ ID NO: 2151) and SGDRSGYS(pS)PGSPG(pT)PGSRSRTPS (SEQ ID NO: 2152), e.g., wherein binding to the latter peptide is at least 2 times (e.g., at least 3 times, at least 4 times, at least 5 times, 2-6 times, 2-5 times, 2-4 times, 2-3 times, 3-5 times or 4-5 times) stronger than background (e.g., non-specific) level of binding, e.g., binding by hIgG1 isotype control); or (n) peptides comprising or consisting of the amino acid sequences SGDRSGYS(pS)PG(pS)PGTPGSRSRTPS (SEQ ID NO: 2150), SGDRSGYSSPG(pS)PG(pT)PGSRSRTPS (SEQ ID NO: 2151), SGDRSGYS(pS)PGSPG(pT)PGSRSRTPS (SEQ ID NO: 2152), and SGDRSGYS(pS)PG(pS)PG(pT)PGSRSRTPS (SEQ ID NO: 2149) (e.g., wherein binding to the peptides is at least 1.6 times stronger (e.g., at least 1.7 times, at least 1.8 times, at least 1.9 times, at least 2 times, at least 3 times, 1.6-4 times, 1.6-3 times stronger) than background (e.g., non-specific) level of binding, e.g., binding by hIgG1 isotype control); wherein p(S) and p(T) correspond to a phosphorylated serine and phosphorylated threonine, respectively, optionally wherein binding is assessed, e.g., using one point ELISA. In some embodiments, the antibody comprises (a) a heavy chain variable region (VH) comprising CDR1, CDR2, and CDR3 sequences comprising SEQ ID NOs: 2200, 2201, and 2202, respectively, and a light chain variable region (VL) comprising CDR1, CDR2, and CDR3 sequences comprising SEQ ID NOs: 2207, 2208, and 2209, respectively; (b) a heavy chain variable region (VH) comprising CDR1, CDR2, and CDR3 sequences comprising SEQ ID NOs: 2218, 2219, and 2220, respectively, and a light chain variable region (VL) comprising CDR1, CDR2, and CDR3 sequences comprising SEQ ID NOs: 2207, 2208, and 2224, respectively; (c) a heavy chain variable region (VH) comprising CDR1, CDR2, and CDR3 sequences comprising SEQ ID NOs: 2248, 2219, and 2249, respectively, and a light chain variable region (VL) comprising CDR1, CDR2, and CDR3 sequences comprising SEQ ID NOs: 2207, 2208, and 2224, respectively; (d) a heavy chain variable region (VH) comprising CDR1, CDR2, and CDR3 sequences comprising SEQ ID NOs: 2273, 2274, and 2275, respectively, and a light chain variable region (VL) comprising CDR1, CDR2, and CDR3 sequences comprising SEQ ID NOs: 2207, 2208, and 2277, respectively; (e) a heavy chain variable region (VH) comprising CDR1, CDR2, and CDR3 sequences comprising SEQ ID NOs: 2371, 2372, and 2373, respectively, and a light chain variable region (VL) comprising CDR1, CDR2, and CDR3 sequences comprising SEQ ID NOs: 2207, 2208, and 2224, respectively; (f) VH and VL sequences comprising SEQ ID NOs: 2214 and 2215, respectively; (g) VH and VL sequences comprising SEQ ID NOs: 2326 and 2327, respectively; (h) VH and VL sequences comprising SEQ ID NOs: 2253 and 2254, respectively; (i) VH and VL sequences comprising SEQ ID NOs: 2280 and 2281, respectively; or (j) VH and VL sequences comprising SEQ ID NOs: 2377 and 2378, respectively.

In some embodiments, the antibody molecules encoded by the viral genomes of the present disclosure bind to: (a) tau phosphorylated at T217, but not at T212 or T214, or (b) peptides comprising or consisting of the sequences GTPGSRSRTPSLP(pT)PPTRE (SEQ ID NO: 2155) and GTPGSRSRTP(pS)LP(pT)PPTRE (SEQ ID NO: 2158), but not peptides comprising or consisting of the sequences GTPGSRSR(pT)PSLPTPPTRE (SEQ ID NO: 2153), GTPGSRSRTP(pS)LPTPPTRE (SEQ ID NO: 2154), and GTPGSRSR(pT)P(pS)LPTPPTRE (SEQ ID NO: 2156), wherein p(S) and p(T) correspond to a phosphorylated serine and phosphorylated threonine, respectively, optionally wherein binding of the antibody to tau or the peptide is at least 1.5 times stronger (e.g., at least 1.6 times, at least 1.7 times, at least 1.8 times, at least 1.9 times, at least 2 times, at least 3 times, at least 4 times, at least 5 times, at least 6 times, 1.5-4 times, 1.5-3, 4-6 times stronger) than background (non-specific) level of binding, e.g., binding by hIgG1 isotype control), and optionally wherein binding of the antibody to tau or the peptide is assessed, e.g., using one point ELISA as described, e.g., in Example 8. In some embodiments, the antibody comprises: (a) a heavy chain variable region (VH) comprising CDR1, CDR2, and CDR3 sequences comprising SEQ ID NOs: 2232, 2233, and 2234, respectively, and a light chain variable region (VL) comprising CDR1, CDR2, and CDR3 sequences comprising SEQ ID NOs: 2238, 2239, and 2240, respectively; (b) a heavy chain variable region (VH) comprising CDR1, CDR2, and CDR3 sequences comprising SEQ ID NOs: 2284, 2392, and 2393, respectively, and a light chain variable region (VL) comprising CDR1, CDR2, and CDR3 sequences comprising SEQ ID NOs: 2404, 2405, and 2406, respectively; (c) a heavy chain variable region (VH) comprising CDR1, CDR2, and CDR3 sequences comprising SEQ ID NOs: 2445, 2446, and 2447, respectively, and a light chain variable region (VL) comprising CDR1, CDR2, and CDR3 sequences comprising SEQ ID NOs: 2452, 2421, and 2453, respectively; (d) VH and VL sequences comprising SEQ ID NOs: 2244 and 2245, respectively; (e) VH and VL sequences comprising SEQ ID NOs: 2411 and 2412, respectively; or (f) VH and VL sequences comprising SEQ ID NOs: 2455 and 2456, respectively.

Specific binding to the aforementioned recited phosphorylated residues or peptides can be determined by comparison with the background level of binding of the assay (e.g., one point ELISA) or level of binding by a negative control, such as an isotype control antibody (e.g., a human IgG1 isotype control antibody).

Anti-tau antibodies encoded by the viral genomes of the present disclosure may target tau in any antigenic form. As non-limiting examples, antigenic tau may be an unphosphorylated or unmodified tau protein, a phosphorylated or otherwise post-translationally modified tau protein (O-GlnAcylated, or nitrosylated), an oligomeric species of tau protein, a soluble species of tau protein, an insoluble species of tau protein, a conformationally abnormal species of tau protein, a neuropathological form of tau protein and/or a neurofibrillary tangle or a precursor thereof.

Anti-tau antibody molecules encoded by the viral genomes, may target any antigenic region or epitope along the full length of any of the six human tau protein isoforms, such as, but not limited to, tau441 (NP_005901.2; MAEPRQEFEVMEDHAGTYGLGDRKDQGGYTMHQDQEGDTDAGLKESPLQTPTEDGSEEPGSET SDAKSTPTAEDVTAPLVDEGAPGKQAAAQPHTEIPEGTTAEEAGIGDTPSLEDEAAGHVTQARM VSKSKDGTGSDDKKAKGADGKTKIATPRGAAPPGQKGQANATRIPAKTPPAPKTPPSSGEPPKSG DRSGYSSPGSPGTPGSRSRTPSLPTPPTREPKKVAVVRTPPKSPSSAKSRLQTAPVPMPDLKNVKS KIGSTENLKHQPGGGKVQIINKKLDLSNVQSKCGSKDNIKHVPGGGSVQIVYKPVDLSKVTSKCG SLGNIHHKPGGGQVEVKSEKLDFKDRVQSKIGSLDNITHVPGGGNKKIETHKLTFRENAKAKTD HGAEIVYKSPVVSGDTSPRHLSNVSSTGSIDMVDSPQLATLADEVSASLAKQGL; SEQ ID NO: 2127). As non-limiting examples, the targeted antigenic peptides of the tau protein may be any of the following phosphorylated sites pT50, pS396, pS396-pS404, pS404, pS396-pS404-pS422, pS409, pS413, pS422, pS198, pS199, pS199-pS202, pS202, pT205, pT212, pS214, pT212-pS214, pT181, pT231, cis-pT231, pT231-pS235, pS235, pS238, pT245, pS262, pY310, pY394, pS324, pS356, pTau¹⁷⁷⁻¹⁸⁷, pS396-pS404, pY18, pS610, pS622, nitrosylated tau (nY18, nY29), methylated tau (di-meK281, dimeK311), 0-GlnAcylated tau at S400, any of the following acetylated sites acK174, acK274, acK280, acK281 and/or any combination thereof. Acetylated tau proteins and associated antigenic peptides are described in Min et al., 2010, Neuron., 67, 953-966, Min et al., 2015, Nature Medicine., 10, 1154-1162, Cohen et al., 2011, Nature Communications., 2, 252, Gorsky et al., 2016, Scientific Report., 6, 22685, Tracy et al., 2016, Neuron., 90, 245-260, the contents of each of which are herein incorporated by reference in their entirety. Phosphorylated tau proteins and associated antigenic peptides are described in Asuni et al., 2007, J Neurosci., 27, 9115-9129, Boutajangout et al., 2010, J Neurosci., 30, 16559-16566, Boutajangout et al., 2011, J Neurochem., 118, 658-667, Chai et al., 2011, J Biol Chem., 286, 34457-34467, Gu et al., 2011, J Biol Chem., 288, 33081-33095, Sankaranarayanan et al., 2015, PLoS One, 10, e0125614, Ittner et al., 2015, J Neurochem., 132, 135-145, D'Abramo et al., 2016, Neurobiol Aging., 37, 58-65, Collin et al., 2014, Brain., 137, 2834-2846, Kondo et al., 2015, Nature., 523, 431-436, the contents of each of which are herein incorporated by reference in their entirety.

In some embodiments, the targeted antigenic peptides of the tau protein may comprise a sequence selected from SPGTPGSRSRpTPSLPTPP (SEQ ID NO: 2128), SRTPSLPpTPPTREPK (SEQ ID NO: 2129), KKVAVVRpTPPKSPSS (SEQ ID NO: 2130), TPGSRSRpTPSLPpTPPTREPK (SEQ ID NO: 2131), SRTPSLPpTPPTREPKKVAVVRpTPPKSPSS (SEQ ID NO: 2132), TPGSRSRpTPSLPpTPPTREPKKVAVVRpTPPKSPSS (SEQ ID NO: 2133), TPGSRSRTPSLPTPPTREPKKVAVVRTPPKSPSS (SEQ ID NO: 2134), TPGSRSRpTPSLPTPPTREPKKVAVVRpTPPKSPSS (SEQ ID NO: 2135), and TPPSSGEPPKSGDRSGYSSPGSPGTPGSRSRTPSLPTPPTREPKKVAVV (SEQ ID NO: 2136).

In some embodiments, the antibody molecule encoded by the viral genomes of the present disclosure may be a pS409 targeting antibody as described in Lee et al., 2016, Cell Reports, 16, 1690-1700, or International Patent Publication WO2013151762, the contents of each of which are herein incorporated by reference in their entirety. In some embodiments, this antibody may be RG6100 or R071057 or variants or fragments thereof.

In some embodiments, the antibody molecule encoded by the viral genomes of the present disclosure may be a pS413 targeting antibody as described in Umeda et al., 2015, Ann Clin Trans Neurol., 2(3), 241-255 or International Patent Publication WO2013180238, the contents of each of which are herein incorporated by reference in their entirety. In some embodiments, the antibody is Ta1505 or variants or fragments thereof.

In some embodiments, the antibody molecule encoded by the viral genomes of the present disclosure may target a tau epitope with amino acid residues 210-275, more specifically pS238 and/or pT245, as described in International Publication WO2011053565, the contents of which are herein incorporated by reference in their entirety.

In some embodiments, the CDRs of an antibody molecule encoded by the viral genomes of the present disclosure may be any of those listed in or incorporated in the antibody sequences of Table 7-16. In some embodiments, the CDRs may be any of those described in International Publication WO2015122922, the contents of which are herein incorporated by reference in their entirety. In some embodiments, a CDR may be any of those chosen from the group of SEQ ID NO: 41, 49, or 57 of WO2015122922. Further a CDR of an antibody encoded by the viral genomes of the present disclosure may have 50%, 60%, 70%, 80%, 90%, or 95% identity to SEQ ID NO: 41, 49, or 57 of WO2015122922.

In some embodiments, the antibody molecules encoded by the viral genomes of the present disclosure may be any of those described in International Publication WO2016097315, the contents of which are herein incorporated by reference in their entirety. In some embodiments, the antibody may have an amino acid sequence as shown by SEQ ID NO: 2, 11, 20, 29, 38, 47, 56, 65, 74, 83, 92, 101, 110, 119, 128, 137, 146, 155, 164, 173, 182, 191, 209, 218, 226, or 227 of WO2016097315.

In some embodiments, the antibody molecules encoded by the viral genomes of the present disclosure may be a multispecific blood brain barrier receptor antibody that also targets tau, as described in International Publication WO2016094566, the contents of which are herein incorporated by reference in their entirety. In some embodiments, the antibody may have a sequence as shown by SEQ ID NO: 1, 2, 17, 18, 33, 34, 49, 50, 65, 66, 81, 82, 9-16, 25-32, 41-48, 57-64, 73-80, 89-96 of WO2016094566.

In some embodiments, the antibody molecules encoded by the viral genomes of the present disclosure may be any of those taught in United States Patent Nos. U.S. Pat. Nos. 8,778,343 and 9,125,846, International Publications WO2012051498 and WO2011026031, or United States Publication Nos. US20150004169 and US20150322143, the contents of each of which are herein incorporated by reference in their entirety. Such antibodies may include those that bind to oligomeric species of tau. Further, such an antibody may be referred to as TOMA (tau oligomer monoclonal antibody), as described in Castillo-Carranza et at (Castillo-Carranza, D L et al., 2014 J Neurosci 34(12)4260-72) the contents of which are herein incorporated by reference in their entirety. In some embodiments, the antibody that binds oligomeric tau may be TTC-99.

In some embodiments, the antibody molecules encoded by the viral genomes of the present disclosure may be any of those taught in International Publications WO2014059442, the contents of which are herein incorporated by reference in their entirety. Such antibodies may include those that bind to oligomeric species of tau.

In some embodiments, the antibody molecules encoded by the viral genomes of the present disclosure may be any of those taught in the International Publications WO2014008404 and WO2016126993, United States Patent Publication US20150183855, Yanamandra, K et al., 2013 Neuron 80(2):402-14 and Yanamandra, K et al 2015 Ann Clin Transl Neurol 2(3):278-88, the contents of each of which are herein incorporated by reference in their entirety. Such antibodies may block tau seeding. Non-limiting examples of antibodies described in these publications include HJ8.1.1, HJ8.1.2, HJ8.2, HJ8.3, HJ8.4, HJ8.5, HJ8.7, HJ8.8, HJ9.1, HJ9.2, HJ9.3, HJ9.4, HJ9.5, and variants thereof. Non-limiting examples of targeted epitopes of tau may include amino acids 22-34, 385-391, 405-411, 3-6, 118-122, 386-401, 7-13, and/or 272-281 of human tau.

In some embodiments, the antibody molecules encoded by the viral genomes of the present disclosure may be any of those taught in the International Publications WO2002062851, the contents of which are herein incorporated by reference in their entirety.

In some embodiments, the antibody molecules encoded by the viral genomes of the present disclosure may be as described in Bright, J et al., 2015 Neurobiol of Aging 36:693-709; Pedersen, JT and Sigurdsson E M, 2015 Trends Mol Med 21(6):394-402; Levites, Y et al 2015 J Neurosci 35(16)6265-76; Jicha et al 1999 J Neurosci 19(17):7486-94; Reyes J F et al., 2012 Acta Neuropathol 123(1):119-32; Reynolds M R, et al., 2006 J Neurosci 26(42):10636-45; Gamblin, T C et al 2003 PNAS 100(17):10032-7; Castillo-Carranza, D L et al., 2014 J Neurosci 34(12)4260-72; Walls, K C et al., 2014 Neurosci Lett 575:96-100; Yanamandra, K et al., 2013 Neuron 80(2):402-14; Yanamandra, K et al 2015 Ann Clin Transl Neurol 2(3):278-88; Allen B, et al., 2002 J Neurosci 22(21):9340-51; Gotz, J et al., 2010 Biochem Biophys Acta 1802(10):860-71; Hasegawa, M et al 1996 FEBS Lett 384:25-30; Carmel, G et al 1996 J Biol Chem 271(51):32780-32795; Jicha, G A et al, 1997 J Neurosci Res 48(2):128-132; Jicha, G A et al., 1997 J Neurochem 69(5):2087-95; the contents of each of which are herein incorporated by reference in their entirety.

Anti-tau antibody molecules encoded by the viral genomes of the present disclosure may be any commercially available anti-tau antibody known in the art or developed by a person with skill in the art. Non-limiting examples of commercially available anti-tau antibodies include EPR2396(2) (pThr⁵⁰; Abcam, Cambridge, MA), 5H911 (pThr¹⁸¹; ThermoFisher, Waltham, MA), M7004D06 (pThr¹⁸¹; BioLegend, San Diego, CA), 1E7 (pThr¹⁸¹; EMD Millipore, Billerica, MA), EPR2400 (pSer¹⁹⁸; Abcam, Cambridge, MA), EPR2401Y (pSer¹⁹⁹; Abcam, Cambridge, MA), 2H23L4 (pSer¹⁹⁹; ThermoFisher, Waltham, MA), EPR2402 (pSer²¹²; Abcam, Cambridge, MA), 10F8 (pSer²¹²; Abcam, Cambridge, MA), EPR2403(2) (pThr²⁰⁵; Abcam, Cambridge, MA), EPR1884(2) (pSer²¹⁴; Abcam, Cambridge, MA), EPR2488 (pThr²³¹; Abcam, Cambridge, MA), 1H6L6 (pThr²³¹; ThermoFisher, Waltham, MA), 3G3 (pThr²³¹, pSer²³⁵; Abcam, Cambridge, MA), EPR2452 (pSer²³⁵; Abcam, Cambridge, MA), 12G10 (pSer²³⁸; Abcam, Cambridge, MA), EPR2454 (pSer²⁶²; Abcam, Cambridge, MA), EPR2457(2) (pSer³²⁴; Abcam, Cambridge, MA), EPR2603 (pSer³⁵⁶; Abcam, Cambridge, MA), EPR2731 (pSer³⁹⁶; Abcam, Cambridge, MA), EPR2605 (pSer⁴⁰⁴; Abcam, Cambridge, MA), EPR2866 (pSer⁴²²; Abcam, Cambridge, MA), 1A4 (pTau¹⁷⁷⁻¹⁸⁷; Origene, Rockville, MD), 7G9 (pTau¹⁷⁷⁻¹⁸⁷; Origene, Rockville, MD), 9B4 (pTau¹⁷⁷⁻¹⁸⁷; Origene, Rockville, MD), 2A4 (pTau¹⁷⁷⁻¹⁸⁷; Origene, Rockville, MD), 9G3 (pTyr¹⁸; NovusBio, Littleton, CO), EPR2455(2) (pSer⁶¹⁰; Abcam, Cambridge, MA), EP2456Y (pSer⁶²²; Abcam, Cambridge, MA; EMD Millipore, Billerica, MA), SMI 51 (PHF Tau⁹⁵⁻¹⁰⁸; BioLegend, San Diego, CA), TOMA-1 (Oligomeric Tau; EMD Millipore, Billerica, MA), Tau-nY18 (nTyr¹⁸; Origene, Rockville, MD; BioLegend, San Diego, CA; EMD Millipore, Billerica, MA), Tau-nY29 (nTyr²⁹; BioLegend, San Diego, CA; EMD Millipore, Billerica, MA; Abcam, Cambridge, MA), 1C9.G6 (di-methyl-Lys²⁸¹; BioLegend, San Diego, CA), 7G5.F4 (di-methyl-Lys³¹¹; BioLegend, San Diego, CA), TNT-1 (Tau²¹⁸; EMD Millipore, Billerica, MA), TNT-2 (Tau²⁻¹⁸; EMD Millipore, Billerica, MA), 7B8 (Tau⁵⁻¹²; Abcam, Cambridge, MA), Tau-13 (Tau²⁰⁻³⁵; BioLegend, San Diego, CA), 1-100 (Tau¹⁻¹⁰⁰; BioLegend, San Diego, CA), 2G9.F10 (Tau¹⁵⁷⁻¹⁶⁸; BioLegend, San Diego, CA; Origene, Rockville, MD), 39E10 (Tau¹⁸⁹⁻¹⁹⁵; BioLegend, San Diego, CA; Origene, Rockville, MD), 77E9 (Tau¹¹⁵⁻¹⁹⁵; BioLegend, San Diego, CA; Origene, Rockville, MD), AT8 (pSer²⁰², pThr²⁰⁵; ThermoFisher, Waltham, MA), AT100 (pThr²¹², pSer²¹⁴; ThermoFisher, Waltham, MA), PHF-6 (pThr²³¹; NovusBio, Littleton, CO; EMD Millipore, Billerica, MA; BioLegend, San Diego, CA; ThermoFisher, Waltham, MA), AT180 (pThr²³¹; ThermoFisher, Waltham, MA), AT270 (pThr¹⁸¹; ThermoFisher, Waltham, MA), PHF-13 (pSer³⁹⁶; ThermoFisher, Waltham, MA; BioLegend, San Diego, CA), TauC3 (Asp⁴²¹; BioLegend, San Diego, CA; EMD Millipore, Billerica, MA; ThermoFisher, Waltham, MA), Tau12 (Tau⁶⁻¹⁸; BioLegend, San Diego, CA; EMD Millipore, Billerica, MA), Tau5 (Tau²¹⁰⁻²⁴¹; BioLegend, San Diego, CA; EMD Millipore, Billerica, MA; Abcam, Cambridge MA; ThermoFisher, Waltham, MA), HT7 (Tau¹⁵⁹⁻¹⁶³; ThermoFisher, Waltham, MA), 77G7 (Tau³¹⁶⁻³⁵⁵; BioLegend, San Diego, CA), Tau46 (Tau⁴⁰⁴⁻⁴⁴¹; BioLegend, San Diego, CA; NovusBio, Littleton, CO; Abcam, Cambridge, MA), UMAB239 (Tau⁶²³⁻⁷⁵⁸; Origene, Rockville, MD), OTI6G3 (Tau⁶²³⁻⁷⁵⁸; Origene, Rockville, MD), OTI13E11 (Tau⁶²³⁻⁷⁵⁸; Origene, Rockville, MD), OTI113B5 (Tau⁶²³⁻⁷⁵⁸; Origene, Rockville, MD), E178 (Tau⁷⁰⁰⁻⁸⁰⁰; Abcam, Cambridge, MA), SP70 (N-terminal Tau; Origene, Rockville, MD; NovusBio, Littleton, CO; ThermoFisher, Waltham, MA; Abcam, Cambridge, MA), C45 (N-terminal Tau; Origene, Rockville, MD), Tau7 (C-terminal Tau; EMD Millipore, Billerica, MA), S.125.0 (C-terminal Tau; ThermoFisher, Waltham, MA), 8E6/C11 (Three-repeat Tau²⁰⁹⁻²²⁴; EMD Millipore, Billerica, MA), 1E1/A6 (Four-repeat Tau²⁷⁵⁻²⁹¹; EMD Millipore, Billerica, MA), 7D12.1 (Four-repeat Tau²⁷⁵⁻²⁹¹; EMD Millipore, Billerica, MA), 5C7 (Four-repeat Tau²⁶⁷⁻²⁷⁸; BioLegend, San Diego, CA; Origene, Rockville, MD), 5F9 (Four-repeat Tau²⁷⁵⁻²⁹¹; BioLegend, San Diego, CA; Origene, Rockville, MD), 3H6.H7 (0N Tau³⁹⁻⁵⁰; BioLegend, San Diego, CA; Origene, Rockville, MD), 4H5.B9 (1N Tau⁶⁸⁻⁷⁹; BioLegend, San Diego, CA; Origene, Rockville, MD), 71C11 (2N Tau; BioLegend, San Diego, CA), PC1C6 (unphosphorylated tau; EMD Millipore, Billerica, MA), Tau2 (BioLegend, San Diego, CA; Origene, Rockville, MD; EMD Millipore, Billerica, MA), 2E9 (Origene, Rockville, MD; NovusBio, Littleton, CO), 4F1 (Origene, Rockville, MD; NovusBio, Littleton, CO), 5B10 (NovusBio, Littleton, CO); 5E2 (EMD Millipore, Billerica, MA), Tau-93 (Origene, Rockville, MD), T14 (ThermoFisher, Waltham, MA), T46 (ThermoFisher, Waltham, MA), BT2 (ThermoFisher, Waltham, MA) and/or variants or derivates thereof.

In some embodiments, the antibody molecule encoded by the viral genomes of the present disclosure may be multispecific antibodies for transferrin receptor and a brain antigen, wherein the brain antigen may be tau, as described in International Publication WO2016081643, the contents of which are herein incorporated by reference in their entirety. In some embodiments, the antibody may have a sequence as given by SEQ ID NO: 160 or 161 of WO2016081643.

In some embodiments, the antibody molecules encoded by the viral genomes of the present disclosure are any of those described in United States Patent Nos. U.S. Pat. Nos. 8,871,447, 8,420,613, International Publication No. WO2014193935, WO2010011999, or in United States Publication Nos. US20110250217, US20110020237, US20100316590, or US20120225864, the contents of each of which are herein incorporated by reference in their entirety. In some embodiments, the antibody molecule recognizes a misfolded, amyloidogenic or aggregating protein.

In some embodiments, the antibody molecules encoded by the viral genomes of the present disclosure bind to tau protein epitopes that are recognized by the anti-tau antibody molecules described herein. In some embodiments, anti-tau antibody molecules bind to tau protein epitopes that include a region formed by a complex of at least two tau proteins.

In some embodiments, the antibody molecules encoded by the viral genomes of the present disclosure competes for binding to tau with the aforesaid anti-tau antibody molecules. In some embodiments, the anti-tau antibody molecules encoded by the viral genomes of the present disclosure bind to the same epitope as (e.g., phospho-epitope), substantially the same epitope as, an epitope that overlaps with, or an epitope that substantially overlaps with, the epitope recognized by the aforesaid anti-tau antibody molecules.

In some embodiments, compete or cross-compete refers to the ability of an antibody molecule to interfere with binding of an anti-tau antibody molecule, e.g., an anti-tau antibody molecule described herein, to a target, e.g., tau protein. The interference with binding can be direct or indirect (e.g., through an allosteric modulation of the antibody molecule or the target). The extent to which an antibody molecule is able to interfere with the binding of another antibody molecule to the target, and therefore whether it can be said to compete, can be determined using a competition binding assay, for example, a FACS assay, an ELISA or BIACORE assay. In some embodiments, a competition binding assay is a quantitative competition assay. In some embodiments, a first anti-tau antibody molecule is said to compete for binding to the target with a second anti-tau antibody molecule when the binding of the first antibody molecule to the target is reduced by 10% or more, e.g., 20% or more, 30% or more, 40% or more, 50% or more, 55% or more, 60% or more, 65% or more, 70% or more, 75% or more, 80% or more, 85% or more, 90% or more, 95% or more, 98% or more, 99% or more in a competition binding assay.

In some embodiments, an epitope comprises the moieties of an antigen (e.g., a tau protein antigen) that specifically interact with an antibody molecule. Such moieties, referred to herein as epitopic determinants, typically comprise, or are part of, elements such as amino acid side chains or sugar side chains. An epitopic determinate can be defined by methods known in the art or disclosed herein, e.g., by crystallography or by hydrogen-deuterium exchange. At least one or some of the moieties on the antibody, that specifically interact with an epitopic determinant, are typically located in a CDR(s). Typically, an epitope has a specific three dimensional structural characteristics. Typically, an epitope has specific charge characteristics. Some epitopes are linear epitopes while others are conformational epitopes.

In an embodiment, an epitopic determinant is a moiety on the antigen, e.g., such as amino acid side chain or sugar side chain, or part thereof, which, when the antigen and antibody molecule are co-crystallized, is within a predetermined distance, e.g., within 5 Angstroms, of a moiety on the antibody molecule, referred to herein as a crystallographic epitopic determinant. In some embodiments, the crystallographic epitopic determinants of an epitope are collectively referred to as a crystallographic epitope.

A first antibody molecule binds the same epitope as a second antibody (e.g., a reference antibody molecule, e.g., an antibody molecule disclosed herein) if the first antibody molecule specifically interacts with the same epitopic determinants on the antigen as does the second or reference antibody molecule, e.g., when interaction is measured in the same way for both the antibody molecule and the second or reference antibody molecule. Epitopes that overlap share at least one epitopic determinant. A first antibody molecule binds an overlapping epitope with a second antibody molecule (e.g., a reference antibody molecule, e.g., an antibody molecule disclosed herein) when both antibody molecules specifically interact with a common epitopic determinant. A first and a second antibody molecule (e.g., a reference antibody molecule, e.g., an antibody molecule disclosed herein) bind substantially overlapping epitopes if at least half of the epitopic determinants of the second or reference antibody molecule are found as epitopic determinants in the epitope of the first antibody molecule. A first and a second antibody molecule (e.g., a reference antibody molecule, e.g., an antibody molecule disclosed herein) bind substantially the same epitope if the first antibody molecule binds at least half of the core epitopic determinants of the epitope of the second or reference antibody molecule, wherein the core epitopic determinants are defined by crystallography.

### Payload Component: SH3-Derived Binding Polypeptide

In some embodiments, the nucleic acid encoding the payload, e.g., antibody molecule, comprises a nucleotide sequence encoding a Fyn SH3-derived binding polypeptide (e.g., a Fynomer sequence or Fynomer sequence region herein). Fynomers are small 7-kDa globular proteins derived from the SH3 domain of the human Fyn kinase (Fyn SH3) that can be engineered to bind with antibody-like affinity and specificity to a target of choice through mutation of two loops (RT- and src-loop) on the surface of the Fyn SH3 domain (Brack et al. (2014), Mol Cancer Ther 13(8):2030-2039). Fynomers may be used to generate multispecific FynomAbs, which are obtained by fusion of Fynomers to any of the four antibody light- or heavy-chain termini. Id.

### Payload Component: Linker

In some embodiments, a viral genome described herein may be engineered with one or more spacer or linker regions to separate coding or non-coding regions.

In some embodiments, the payload region of the AAV particle may optionally encode one or more linker sequences. In some cases, the linker may be a peptide linker that may be used to connect the polypeptides encoded by the payload region (i.e., light and heavy antibody chains during expression). Some peptide linkers may be cleaved after expression to separate heavy and light chain domains, allowing assembly of mature antibodies or antibody fragments. Linker cleavage may be enzymatic. In some cases, linkers comprise an enzymatic cleavage site to facilitate intracellular or extracellular cleavage. Some payload regions encode linkers that interrupt polypeptide synthesis during translation of the linker sequence from an mRNA transcript. Such linkers may facilitate the translation of separate protein domains (e.g., heavy and light chain antibody domains) from a single transcript. In some cases, two or more linkers are encoded by a payload region of the viral genome. Non-limiting examples of linkers that may be encoded by the payload region of an AAV particle viral genome are given in Table 19. In some embodiments, the encoded linker comprises an amino acid sequence or nucleic acid sequence encoded by any one of the nucleotide sequences provided in Table 19, or an amino acid sequence or nucleic acid sequence with at least 70%, 75%, 80%, 85%, 90%, 95%, or 99% sequence identity thereto.

TABLE 19
Linkers

| | | | e | |
|---|---|---|---|---|
| Linker | | | ID | |
| ID | Description | Length | NO | Sequence |
| Linker1 | Furin | 12 | 3702 | agaaagaggcga |
| Linker2 | Furin | 12 | 3703 | cgggccaagcgg |
| Linker3 | T2A | 54 | 3704 | gagggcagaggaagtcttctaacatgcggtgacgtggagga |
| | | | | gaatcccggccct |
| Linker4 | F2A | 75 | 3705 | ggaagcggagtgaaacagactttgaattttgaccttctcaa |
| | | | | gttggcgggagacgtggagtccaaccctggacct |
| Linker5 | P2A | 66 | 3706 | ggaagcggagctactaacttcagcctgctgaagcaggctgg |
| | | | | agacgtggaggagaaccctggacct |
| Linker6 | G4S | 18 | 3707 | tccggaggcggcggcagc |
| Linker7 | G4S3 | 45 | 3708 | ggtggtggtggtagcggcggcggcggctctggtggtggtgg |
| | | | | atcc |
| Linker8 | G4S5 | 75 | 3709 | ggcggaggtggctccggaggcggaggcagcggcggaggtgg |
| | | | | gtctggcggaggcgggtgcggcggaggtggctcc |
| Linker9 | IRES | 609 | 3710 | aattccgcccctctcccccccccccctctccctcccccccc |
| | | | | cctaacgttactggccgaagccgcttggaataaggccggtg |
| | | | | tgcgtttgtctatatgttattttccaccatattgccgtctt |
| | | | | ttggcaatgtgagggcccggaaacctggccctgtcttcttg |
| | | | | acgagcattcctaggggtctttcccctctcgccaaaggaat |
| | | | | gcaaggtctgttgaatgtcgtgaaggaagcagttcctctgg |
| | | | | aagcttcttgaagacaaacaacgtctgtagcgaccctttgc |
| | | | | aggcagcggaaccccccacctggcgacaggtgcctctgcgg |
| | | | | ccaaaagccacgtgtataagatacacctgcaaaggcggcac |
| | | | | aaccccagtgccacgttgtgagttggatagttgtggaaaga |
| | | | | gtcaaatggctctcctcaagcgtattcaacaaggggctgaa |
| | | | | ggatgcccagaaggtaccccattgtatgggatctgatctgg |
| | | | | ggcctcggtgcacatgctttacatgtgtttagtcgaggtta |
| | | | | aaaaaacgtctaggccccccgaaccacggggacgtggtttt |
| | | | | cctttgaaaaacacgatgataatatggccacaacc |
| Linker10 | IRES-2 | 623 | 3711 | taacgaattccgcccctctcccccccccccctctccctccc |
| | | | | ccccccctaacgttactggccgaagccgcttggaataaggc |
| | | | | cggtgtgcgtttgtctatatgttattttccaccatattgcc |
| | | | | gtcttttggcaatgtgagggcccggaaacctggccctgtct |
| | | | | tcttgacgagcattcctaggggtctttcccctctcgccaaa |
| | | | | ggaatgcaaggtctgttgaatgtcgtgaaggaagcagttcc |
| | | | | tctggaagcttcttgaagacaaacaacgtctgtagcgaccc |
| | | | | tttgcaggcagcggaacccccacctggcgacaggtgcctc |
| | | | | tgcggccaaaagccacgtgtataagatacacctgcaaaggc |
| | | | | ggcacaaccccagtgccacgttgtgagttggatagttgtgg |
| | | | | aaagagtcaaatggctctcctcaagcgtattcaacaagggg |
| | | | | ctgaaggatgcccagaaggtaccccattgtatgggatctga |
| | | | | tctggggcctcggtgcacatgctttacatgtgtttagtcga |
| | | | | ggttaaaaaaacgtctaggccccccgaaccacggggacgtg |
| | | | | gttttcctttgaaaaacacgatgataatatggccacaaccg |
| | | | | ccgccacc |
| Linker11 | hIgG2 hinge | 54 | 3712 | gtggagaggaagtgctgcgtggagtgcccaccatgccctgc |
| | | | | ccctcctgtggcc |
| Linker12 | hIgG3 hinge | 108 | 3713 | gagctcaaaaccccacttggtgacacaactcacacatgccc |
| | | | | acggtgcccagagcccaaatcttgtgacacacctcccccgt |
| | | | | gcccacggtgcccagcacctgaactc |
| Linker13 | hIgG3-2 hinge | 153 | 3714 | gagctcaaaaccccacttggtgacacaactcacacatgccc |
| | | | | acggtgcccagagcccaaatcttgtgacacacctcccccgt |
| | | | | gcccacggtgcccagagcccaaatcttgtgacacacctccc |
| | | | | ccatgcccacggtgcccagcacctgaactc |
| Linker14 | hIgG3-3 hinge | 198 | 3715 | gagctcaaaaccccacttggtgacacaactcacacatgccc |
| | | | | acggtgcccagagcccaaatcttgtgacacacctcccccgt |
| | | | | gcccacggtgcccagagcccaaatcttgtgacacacctccc |
| | | | | ccatgcccacggtgcccagagcccaaatcttgtgacacacc |
| | | | | tcccccgtgcccaaggtgcccagcacctgaactc |
| Linker15 | msiGG-1 | 45 | 3716 | gtgcccagggattgtggttgtaagccttgcatatgtacagt |
| | hinge | | | ccca |
| Linker16 | msiGG1 hinge | 18 | 3717 | gtgcccagggattgtggt |
| Linker17 | HigG3 hinge | 198 | 3718 | gagctcaaaaccccacttggtgacacaactcacacatgccc |
| | | | | acggtgcccagagcccaaatcttgtgacacacctcccccgt |
| | | | | gcccacggtgcccagagcccaaatcttgtgacacaactcac |
| | | | | acatgcccacggtgcccagagcccaaatcttgtgacacacc |
| | | | | tcccccgtgcccaaggtgcccagcacctgaactc |
| Linker18 | G4S (SEQ ID | 15 | 3719 | ggtggtggtggatcc |
| | NO: 4535) | | | |
| Linker19 | (G4S)2 (SEQ | 30 | 3720 | ggtggtggtggatccggtggtggtggatcc |
| | ID NO: 4539) | | | |
| Linker20 | (G4S)3 (SEQ | 45 | 3721 | ggtggtggtggatccggtggtggtggatccggtggtggtgg |
| | ID NO: 4537) | | | atcc |
| Linker21 | (G4S)4 (SEQ | 60 | 3722 | ggtggtggtggatccggtggtggtggatccggtggtggtgg |
| | ID NO: 4540) | | | atccggtggtggtggatcc |
| Linker22 | (G4S)5 (SEQ | 75 | 3723 | ggtggtggtggtagcggcggcggcggctctggtggtggtgg |
| | ID NO: 4538) | | | atccggtggtggtggatccggtggtggtggatcc |
| Linker23 | (G4S)5 (SEQ | 75 | 3724 | ggtggtggtggatccggtggtggtggatccggtggtggtgg |
| | ID NO: 4538) | | | atccggtggtggtggatccggtggtggtggatcc |
| Linker24 | (G4S)6 | 90 | 3725 | ggtggtggtggtagcggcggcggcggctctggtggtggtgg |
| | | | | atccggtggtggtggatccggtggtggtggatccggtggtg |
| | | | | gtggatcc |
| Linker25 | (G4S)8 | 120 | 3726 | ggtggtggtggtagcggcggcggcggctctggtggtggtgg |
| | | | | atccggtggtggtggatccggtggtggtggatccggtggtg |
| | | | | gtggatccggtggtggtggatccggtggtggtggatcc |
| Linker26 | (G4S)8 | 120 | 3727 | ggtggtggtggatccggtggtggtggatccggtggtggtgg |
| | | | | atccggtggtggtggatccggtggtggtggatccggtggtg |
| | | | | gtggatccggtggtggtggatccggtggtggtggatcc |
| Linker27 | (G4S)4 | 60 | 3728 | ggtggtggtggtagcggcggcggcggctctggtggtggtgg |
| | | | | atccggtggtggtggatcc |
| Linker28 | (G4S)6 | 90 | 3729 | ggtggtggtggatccggtggtggtggatccggtggtggtgg |
| | | | | atccggtggtggtggatccggtggtggtggatccggtggtg |
| | | | | gtggatcc |
| Linker29 | | 15 | 3730 | ggaggaggaggaAGT |
| Linker30 | | | 3731 | ggaggt ggaggt TCT |
| Linker31 | amino acid | | 3732 | GGCTCT |
| Linker32 | | | 3733 | ggtggtggtggatccggaggaggaggaAGTggaggtggagg |
| | | | | tTCT |
| Linker33 | amino acid | | 3734 | GGGGSGGGGSGGGGS |
| | (G4S)3 | | | |

In some embodiments, any of the payloads described herein, can have a linker, e.g. a flexible polypeptide linker, of varying lengths. For example, a (Gly4Ser)n linker, wherein n is 0, 1, 2, 3, 4, 5, 6, 7, or 8 can be used. In some embodiments, the linker comprises a (Gly4Ser)3 (SEQ ID NO: 3734). In some embodiments, the nucleotide sequence encoding the linker comprises the nucleotide sequence of SEQ ID NO: 3708, or a nucleotide sequence having at least one, two, or three but no more than four modifications, e.g., substitutions, relative to SEQ ID NO: 3708. In some embodiments, the encoded linker comprises the amino acid sequence of SEQ ID NO: 3734, or an amino acid sequence having at least one, two, or three but no more than four modifications, e.g., substitutions, relative to SEQ ID NO: 3734.

In some embodiments, any of the antibody molecules described herein can have a linker, e.g. a flexible polypeptide linker, of varying lengths, connecting the variable domains (e.g., the VH and the VL) of the antigen binding domain of the antibody molecule. For example, a (Gly4-Ser)n linker, wherein n is 0, 1, 2, 3, 4, 5, 6, 7, or 8 can be used. In some embodiments, the antibody molecule binds to tau.

In some embodiments, the encoded linker comprises an enzymatic cleavage site, e.g., for intracellular and/or extracellular cleavage. In some embodiments, the linker is cleaved to separate the VH and the VL of the antigen binding domain and/or the heavy chain and light chain of the antibody molecule (e.g., an anti-tau antibody molecule). In some embodiments, the linker is cleaved. In some embodiments, the encoded linker comprises a furin linker or a functional variant. In some embodiments, the nucleotide sequence encoding the furin linker comprises the nucleotide sequence of SEQ ID NO: 3702, a nucleotide sequence with at least 70%, 75%, 80%, 85%, 90%, 95%, or 99% sequence identity to SEQ ID NO: 3702, or a nucleotide sequence having at least one, two, or three but no more than four modifications, e.g., substitutions, relative to SEQ ID NO: 3702. In some embodiments, furin cleaves proteins downstream of a basic amino acid target sequence (e.g., Arg-X-(Arg/Lys)-Arg) (e.g., as described in Thomas, G., 2002. Nature Reviews Molecular Cell Biology 3(10): 753-66; the contents of which are herein incorporated by reference in its entirety). In some embodiments, the encoded linker comprises a 2A self-cleaving peptide (e.g., a 2A peptide derived from foot-and-mouth disease virus (F2A), porcine teschovirus-1 (P2A), Thoseaasigna virus (T2A), or equine rhinitis A virus (E2A)). In some embodiments, the encoded linker comprises a T2A self-cleaving peptide linker. In some embodiments, the nucleotide sequence encoding the T2A linker comprises the nucleotide sequence of SEQ ID NO: 3704, a nucleotide sequence with at least 70%, 75%, 80%, 85%, 90%, 95%, or 99% sequence identity to SEQ ID NO: 3704, or a nucleotide sequence having at least one, two, or three but no more than four modifications, e.g., substitutions, relative to SEQ ID NO: 3704. In some embodiments, the T2A linker comprises an amino acid sequence encoded by SEQ ID NO: 3704, or an amino acid sequence having at least one, two, or three but no more than four modifications, e.g., substitutions, relative to an amino acid sequence encoded by SEQ ID NO: 3704. In some embodiments, the nucleic acid encoding the payload encodes a furin linker and a T2A linker.

In some embodiments, the encoded linker comprises an internal ribosomal entry site (IRES) is a nucleotide sequence (>500 nucleotides) for initiation of translation in the middle of a nucleotide sequence, e.g., an mRNA sequence (Kim, J. H. et al., 2011. PLoS One 6(4): e18556; the contents of which are herein incorporated by reference in its entirety), which can be used, for example, to modulate expression of one or more transgenes. In some embodiments, the encode linker comprises a small and unbranched serine-rich peptide linker, such as those described by Huston et al. in U.S. Pat. No. 5,525,491, the contents of which are herein incorporated in their entirety. In some embodiments, polypeptides comprising a serine-rich linker has increased solubility. In some embodiments, the encoded linker comprises an artificial linker, such as those described by Whitlow and Filpula in U.S. Pat. No. 5,856,456 and Ladner et al. in U.S. Pat. No. 4,946,778, the contents of each of which are herein incorporated by their entirety.

In some embodiments, the encoded linkers comprises a cathepsin, a matrix metalloproteinases or a legumain cleavage sites, such as those described e.g. by Cizeau and Macdonald in International Publication No. WO2008052322, the contents of which are herein incorporated in their entirety.

In some embodiments, the encoded linker comprises an internal ribosomal entry site (IRES) is a nucleotide sequence (>500 nucleotides) for initiation of translation in the middle of a nucleotide sequence, e.g., an mRNA sequence (Kim, J. H. et al., 2011. PLoS One 6(4): e18556; the contents of which are herein incorporated by reference in its entirety), which can be used, for example, to modulate expression of one or more transgenes. In some embodiments, the encode linker comprises a small and unbranched serine-rich peptide linker, such as those described by Huston et al. in U.S. Pat. No. 5,525,491, the contents of which are herein incorporated in their entirety. In some embodiments, polypeptides comprising a serine-rich linker has increased solubility. In some embodiments, the encoded linker comprises an artificial linker, such as those described by Whitlow and Filpula in U.S. Pat. No. 5,856,456 and Ladner et al. in U.S. Pat. No. 4,946,778, the contents of each of which are herein incorporated by their entirety.

In some embodiments, the nucleotide sequence encoding the linker comprises about 10 to about 700 nucleotides in length, e.g., about 10 to about 700 nucleotides, e.g. about 10 to about 100, e.g., about 50-200 nucleotides, about 150-300 nucleotides, about 250-400 nucleotides, about 350-500 nucleotides, about 450-600 nucleotides, about 550-700 nucleotides, about 650-700 nucleotides. In some embodiments, the nucleotide sequence encoding the linker comprises about 5 to about 20 nucleotides in length, e.g., about 12 nucleotides in length. In some embodiments, the nucleotide sequence encoding the linker comprises about 40 to about 60 nucleotides in length, e.g., about 54 nucleotides in length.

### Payload Component: Signal Sequence

In some embodiments, the nucleic acid sequence comprising the transgene encoding the payload, e.g., an antibody molecule, comprises a nucleic acid sequence encoding a signal sequence (e.g., a signal sequence region herein). In some embodiments, the nucleic acid sequence comprising the transgene encoding the payload comprises two signal sequence regions. In some embodiments, the nucleic acid sequence comprising the transgene encoding the payload comprises three or more signal sequence regions.

In some embodiments, the nucleotide sequence encoding the signal sequence is located 5′ relative to the nucleotide sequence encoding the VH and/or the heavy chain. In some embodiments, the nucleotide sequence encoding the signal sequence is located 5′ relative to the nucleotide sequence encoding the VL and/or the light chain. In some embodiments, the encoded VH, VL, heavy chain, and/or light chain of the encoded antibody molecule comprises a signal sequence at the N-terminus, wherein the signal sequence is optionally cleaved during cellular processing and/or localization of the antibody molecule.

### Self-Complementary and Single Strand Vectors

In some embodiments, the AAV vector used in the present disclosure is a single strand vector (ssAAV).

In some embodiments, the AAV vectors may be self-complementary AAV vectors (scAAVs). See, e.g., U.S. Pat. No. 7,465,583. scAAV vectors contain both DNA strands that anneal together to form double stranded DNA. By skipping second strand synthesis, scAAVs allow for rapid expression in the cell.

In some embodiments, the AAV vector used in the present disclosure is a scAAV.

Methods for producing and/or modifying AAV vectors are disclosed in the art such as pseudotyped AAV vectors (International Patent Publication Nos. WO200028004; WO200123001; WO2004112727; WO 2005005610 and WO 2005072364, the content of each of which are incorporated herein by reference in their entirety).

### Genome Size

In one embodiment, the AAV particle described herein (e.g., an AAV particle comprising an AAV capsid polypeptide, e.g., an AAV capsid variant), may comprise a single-stranded or double-stranded viral genome. The size of the viral genome may be small, medium, large or the maximum size. As described above, the viral genome may comprise a promoter and a polyA tail.

In one embodiment, the viral genome may be a small single stranded viral genome. A small single stranded viral genome may be 2.1 to 3.5 kb in size such as, but not limited to, about 2.1, 2.2, 2.3, 2.4, 2.5, 2.6, 2.7, 2.8, 2.9, 3.0, 3.1, 3.2, 3.3, 3.4, and 3.5 kb in size.

In one embodiment, the viral genome may be a small double stranded viral genome. A small double stranded viral genome may be 1.3 to 1.7 kb in size such as, but not limited to, about 1.3, 1.4, 1.5, 1.6, and 1.7 kb in size.

In one embodiment, the viral genome may be a medium single stranded viral genome. A medium single stranded viral genome may be 3.6 to 4.3 kb in size such as, but not limited to, about 3.6, 3.7, 3.8, 3.9, 4.0, 4.1, 4.2 and 4.3 kb in size.

In one embodiment, the viral genome may be a medium double stranded viral genome. A medium double stranded viral genome may be 1.8 to 2.1 kb in size such as, but not limited to, about 1.8, 1.9, 2.0, and 2.1 kb in size.

In one embodiment, the viral genome may be a large single stranded viral genome. A large single stranded viral genome may be 4.4 to 6.0 kb in size such as, but not limited to, about 4.4, 4.5, 4.6, 4.7, 4.8, 4.9, 5.0, 5.1, 5.2, 5.3, 5.4, 5.5, 5.6, 5.7, 5.8, 5.9 and 6.0 kb in size.

In one embodiment, the viral genome may be a large double stranded viral genome. A large double stranded viral genome may be 2.2 to 3.0 kb in size such as, but not limited to, about 2.2, 2.3, 2.4, 2.5, 2.6, 2.7, 2.8, 2.9 and 3.0 kb in size.

### Backbone

In certain embodiments, a cis-element such as a vector backbone is incorporated into the viral particle encoding, e.g., an anti-tau antibody molecule described herein. Without wishing to be bound by theory, it is believed, in some embodiments, the backbone sequence may contribute to the stability of an anti-tau antibody molecule expression, and/or the level of expression of the an anti-tau antibody molecule.

The present disclosure also provides in some embodiments, a nucleic acid encoding a viral genome, an a backbone region suitable for replication of the viral genome in a cell, e.g., a bacterial cell (e.g., wherein the backbone region comprises one or both of a bacterial origin of replication and a selectable marker).

### II. Viral Production

### General Viral Production Process

Cells for the production of AAV, e.g., rAAV, particles may comprise, in some embodiments, mammalian cells (such as HEK293 cells) and/or insect cells (such as Sf9 cells).

In various embodiments, AAV production includes processes and methods for producing AAV particles and vectors which can contact a target cell to deliver a payload, e.g. a recombinant viral construct, which includes a nucleotide encoding a payload molecule. In certain embodiments, the viral vectors are adeno-associated viral (AAV) vectors such as recombinant adeno-associated viral (rAAV) vectors. In certain embodiments, the AAV particles are adeno-associated viral (AAV) particles such as recombinant adeno-associated viral (rAAV) particles.

In some embodiments, disclosed herein is a vector comprising a viral genome of the present disclosure. In some embodiments, disclosed herein is a cell comprising a viral genome of the present disclosure. In some embodiments, the cell is a bacterial cell, a mammalian cell (e.g., a HEK293 cell), or an insect cell (e.g., an Sf9 cell).

In some embodiments, disclosed herein is a method of making a viral genome. The method comprising providing a nucleic acid encoding a viral genome described herein and a backbone region suitable for replication of the viral genome in a cell, e.g., a bacterial cell (e.g., wherein the backbone region comprises one or both of a bacterial origin of replication and a selectable marker), and excising the viral genome from the backbone region, e.g., by cleaving the nucleic acid molecule at upstream and downstream of the viral genome. In some embodiments, the viral genome comprising a promoter operably linked to nucleic acid comprising a transgene encoding an anti-tau antibody molecule (e.g., an anti-tau antibody molecule described herein), will be incorporated into an AAV particle produced in the cell. In some embodiments, the cell is a bacterial cell, a mammalian cell (e.g., a HEK293 cell), or an insect cell (e.g., an Sf9 cell).

In some embodiments, disclosed herein is a method of making a recombinant AAV particle of the present disclosure, the method comprising (i) providing a host cell comprising a viral genome described herein and incubating the host cell under conditions suitable to enclose the viral genome in a capsid protein, e.g., a capsid protein described herein (e.g., a capsid protein listed in Tables 1, 3, 4, and 5, or a capsid protein comprising a peptide listed in Tables 2, 20, 23, and 24), thereby making the recombinant AAV particle. In some embodiments, the method comprises prior to step (i), introducing a first nucleic acid comprising the viral genome into a cell. In some embodiments, the host cell comprises a second nucleic acid encoding the capsid protein. In some embodiments, the second nucleic acid is introduced into the host cell prior to, concurrently with, or after the first nucleic acid molecule. In some embodiments, the host cell is a bacterial cell, a mammalian cell (e.g., a HEK293 cell), or an insect cell (e.g., an Sf9 cell).

In various embodiments, methods are provided herein of producing AAV particles or vectors by (a) contacting a viral production cell with one or more viral expression constructs encoding at least one AAV capsid protein, and one or more payload constructs encoding a payload molecule, which can be selected from: a transgene, a polynucleotide encoding protein, and a modulatory nucleic acid; (b) culturing the viral production cell under conditions such that at least one AAV particle or vector is produced, and (c) isolating the AAV particle or vector from the production stream.

In these methods, a viral expression construct may encode at least one structural protein and/or at least one non-structural protein. The structural protein may include any of the native or wild type capsid proteins VP1, VP2, and/or VP3, or a chimeric protein thereof. The non-structural protein may include any of the native or wild type Rep78, Rep68, Rep52, and/or Rep40 proteins or a chimeric protein thereof.

In certain embodiments, contacting occurs via transient transfection, viral transduction, and/or electroporation.

In certain embodiments, the viral production cell is selected from a mammalian cell and an insect cell. In certain embodiments, the insect cell includes a Spodoptera frugiperda insect cell. In certain embodiments, the insect cell includes a Sf9 insect cell. In certain embodiments, the insect cell includes a Sf21 insect cell.

The payload construct vector of the present disclosure may include, in various embodiments, at least one inverted terminal repeat (ITR) and may include mammalian DNA.

Also provided are AAV particles and viral vectors produced according to the methods described herein.

In various embodiments, the AAV particles of the present disclosure may be formulated as a pharmaceutical composition with one or more acceptable excipients.

In certain embodiments, an AAV particle or viral vector may be produced by a method described herein.

In certain embodiments, the AAV particles may be produced by contacting a viral production cell (e.g., an insect cell or a mammalian cell) with at least one viral expression construct encoding at least one capsid protein and at least one payload construct vector. The viral production cell may be contacted by transient transfection, viral transduction, and/or electroporation. The payload construct vector may include a payload construct encoding a payload molecule such as, but not limited to, a transgene, a polynucleotide encoding protein, and a modulatory nucleic acid. The viral production cell can be cultured under conditions such that at least one AAV particle or vector is produced, isolated (e.g., using temperature-induced lysis, mechanical lysis and/or chemical lysis) and/or purified (e.g., using filtration, chromatography, and/or immunoaffinity purification). As a non-limiting example, the payload construct vector may include mammalian DNA.

In certain embodiments, the AAV particles are produced in an insect cell (e.g., Spodoptera frugiperda (Sf9) cell) using a method described herein. As a non-limiting example, the insect cell is contacted using viral transduction which may include baculoviral transduction.

In certain embodiments, the AAV particles are produced in an mammalian cell (e.g., HEK293 cell) using a method described herein. As a non-limiting example, the mammalian cell is contacted using viral transduction which may include multiplasmid transient transfection (such as triple plasmid transient transfection).

In certain embodiments, the AAV particle production method described herein produces greater than 10¹, greater than 10², greater than 10³, greater than 10⁴, or greater than 10⁵ AAV particles in a viral production cell.

In certain embodiments, a process of the present disclosure includes production of viral particles in a viral production cell using a viral production system which includes at least one viral expression construct and at least one payload construct. The at least one viral expression construct and at least one payload construct can be co-transfected (e.g. dual transfection, triple transfection) into a viral production cell. The transfection is completed using standard molecular biology techniques known and routinely performed by a person skilled in the art. The viral production cell provides the cellular machinery necessary for expression of the proteins and other biomaterials necessary for producing the AAV particles, including Rep proteins which replicate the payload construct and Cap proteins which assemble to form a capsid that encloses the replicated payload constructs. The resulting AAV particle is extracted from the viral production cells and processed into a pharmaceutical preparation for administration.

In various embodiments, once administered, an AAV particle disclosed herein may, without being bound by theory, contact a target cell and enter the cell, e.g., in an endosome. The AAV particles, e.g., those released from the endosome, may subsequently contact the nucleus of the target cell to deliver the payload construct. The payload construct, e.g. recombinant viral construct, may be delivered to the nucleus of the target cell wherein the payload molecule encoded by the payload construct may be expressed.

In certain embodiments, the process for production of viral particles utilizes seed cultures of viral production cells that include one or more baculoviruses (e.g., a Baculoviral Expression Vector (BEV) or a baculovirus infected insect cell (BIIC) that has been transfected with a viral expression construct and a payload construct vector). In certain embodiments, the seed cultures are harvested, divided into aliquots and frozen, and may be used at a later time point to initiate an infection of a naïve population of production cells.

In some embodiments, large scale production of AAV particles utilizes a bioreactor. Without being bound by theory, the use of a bioreactor may allow for the precise measurement and/or control of variables that support the growth and activity of viral production cells such as mass, temperature, mixing conditions (impellor RPM or wave oscillation), CO₂ concentration, O₂ concentration, gas sparge rates and volumes, gas overlay rates and volumes, pH, Viable Cell Density (VCD), cell viability, cell diameter, and/or optical density (OD). In certain embodiments, the bioreactor is used for batch production in which the entire culture is harvested at an experimentally determined time point and AAV particles are purified. In some embodiments, the bioreactor is used for continuous production in which a portion of the culture is harvested at an experimentally determined time point for purification of AAV particles, and the remaining culture in the bioreactor is refreshed with additional growth media components.

In various embodiments, AAV viral particles can be extracted from viral production cells in a process which includes cell lysis, clarification, sterilization and purification. Cell lysis includes any process that disrupts the structure of the viral production cell, thereby releasing AAV particles. In certain embodiments, cell lysis may include thermal shock, chemical, or mechanical lysis methods. Clarification can include the gross purification of the mixture of lysed cells, media components, and AAV particles. In certain embodiments, clarification includes centrifugation and/or filtration, including but not limited to depth end, tangential flow, and/or hollow fiber filtration.

In various embodiments, the end result of viral production is a purified collection of AAV particles which include two components: (1) a payload construct (e.g. a recombinant AAV vector genome construct) and (2) a viral capsid.

In certain embodiments, a viral production system or process of the present disclosure includes steps for producing baculovirus infected insect cells (BIICs) using Viral Production Cells (VPC) and plasmid constructs. Viral Production Cells (VPCs) from a Cell Bank (CB) are thawed and expanded to provide a target working volume and VPC concentration. The resulting pool of VPCs is split into a Rep/Cap VPC pool and a Payload VPC pool. One or more Rep/Cap plasmid constructs (viral expression constructs) are processed into Rep/Cap Bacmid polynucleotides and transfected into the Rep/Cap VPC pool. One or more Payload plasmid constructs (payload constructs) are processed into Payload Bacmid polynucleotides and transfected into the Payload VPC pool. The two VPC pools are incubated to produce P1 Rep/Cap Baculoviral Expression Vectors (BEVs) and P1 Payload BEVs. The two BEV pools are expanded into a collection of Plaques, with a single Plaque being selected for Clonal Plaque (CP) Purification (also referred to as Single Plaque Expansion). The process can include a single CP Purification step or can include multiple CP Purification steps either in series or separated by other processing steps. The one-or-more CP Purification steps provide a CP Rep/Cap BEV pool and a CP Payload BEV pool. These two BEV pools can then be stored and used for future production steps, or they can be then transfected into VPCs to produce a Rep/Cap BIIC pool and a Payload BIIC pool.

In certain embodiments, a viral production system or process of the present disclosure includes steps for producing AAV particles using Viral Production Cells (VPC) and baculovirus infected insect cells (BIICs). Viral Production Cells (VPCs) from a Cell Bank (CB) are thawed and expanded to provide a target working volume and VPC concentration. The working volume of Viral Production Cells is seeded into a Production Bioreactor and can be further expanded to a working volume of 200-2000 L with a target VPC concentration for BIIC infection. The working volume of VPCs in the Production Bioreactor is then co-infected with Rep/Cap BIICs and Payload BIICs, with a target VPC:BIIC ratio and a target BIIC:BIIC ratio. VCD infection can also utilize BEVs. The co-infected VPCs are incubated and expanded in the Production Bioreactor to produce a bulk harvest of AAV particles and VPCs.

### Viral Expression Constructs

In various embodiments, the viral production system of the present disclosure includes one or more viral expression constructs that can be transfected/transduced into a viral production cell. In certain embodiments, a viral expression construct or a payload construct of the present disclosure can be a bacmid, also known as a baculovirus plasmid or recombinant baculovirus genome. In certain embodiments, the viral expression includes a protein-coding nucleotide sequence and at least one expression control sequence for expression in a viral production cell. In certain embodiments, the viral expression includes a protein-coding nucleotide sequence operably linked to least one expression control sequence for expression in a viral production cell. In certain embodiments, the viral expression construct contains parvoviral genes under control of one or more promoters. Parvoviral genes can include nucleotide sequences encoding non-structural AAV replication proteins, such as Rep genes which encode Rep52, Rep40, Rep68, or Rep78 proteins. Parvoviral genes can include nucleotide sequences encoding structural AAV proteins, such as Cap genes which encode VP1, VP2, and VP3 proteins.

Viral expression constructs of the present disclosure may include any compound or formulation, biological or chemical, which facilitates transformation, transfection, or transduction of a cell with a nucleic acid. Exemplary biological viral expression constructs include plasmids, linear nucleic acid molecules, and recombinant viruses including baculovirus. Exemplary chemical vectors include lipid complexes. Viral expression constructs are used to incorporate nucleic acid sequences into virus replication cells in accordance with the present disclosure. (O'Reilly, David R., Lois K. Miller, and Verne A. Luckow. Baculovirus expression vectors: a laboratory manual. Oxford University Press, 1994.); Maniatis et al., eds. Molecular Cloning. CSH Laboratory, NY, N.Y. (1982); and, Philiport and Scluber, eds. Liposomes as tools in Basic Research and Industry. CRC Press, Ann Arbor, Mich. (1995), the contents of each of which are herein incorporated by reference in their entirety as related to viral expression constructs and uses thereof.

In certain embodiments, the viral expression construct is an AAV expression construct which includes one or more nucleotide sequences encoding non-structural AAV replication proteins, structural AAV capsid proteins, or a combination thereof.

In certain embodiments, the viral expression construct of the present disclosure may be a plasmid vector. In certain embodiments, the viral expression construct of the present disclosure may be a baculoviral construct.

The present disclosure is not limited by the number of viral expression constructs employed to produce AAV particles or viral vectors. In certain embodiments, one, two, three, four, five, six, or more viral expression constructs can be employed to produce AAV particles in viral production cells in accordance with the present disclosure. In certain embodiments of the present disclosure, a viral expression construct may be used for the production of an AAV particles in insect cells. In certain embodiments, modifications may be made to the wild type AAV sequences of the capsid and/or rep genes, for example to improve attributes of the viral particle, such as increased infectivity or specificity, or to enhance production yields.

In certain embodiments, the viral expression construct may contain a nucleotide sequence which includes start codon region, such as a sequence encoding AAV capsid proteins which include one or more start codon regions. In certain embodiments, the start codon region can be within an expression control sequence. The start codon can be ATG or a non-ATG codon (i.e., a suboptimal start codon where the start codon of the AAV VP1 capsid protein is a non-ATG).

In certain embodiments, the viral expression construct used for AAV production may contain a nucleotide sequence encoding the AAV capsid proteins where the initiation codon of the AAV VP1 capsid protein is a non-ATG, i.e., a suboptimal initiation codon, allowing the expression of a modified ratio of the viral capsid proteins in the production system, to provide improved infectivity of the host cell. In a non-limiting example, a viral construct vector may contain a nucleic acid construct comprising a nucleotide sequence encoding AAV VP1, VP2, and VP3 capsid proteins, wherein the initiation codon for translation of the AAV VP1 capsid protein is CTG, TTG, or GTG, as described in U.S. Pat. No. 8,163,543, the contents of which are herein incorporated by reference in their entirety as related to AAV capsid proteins and the production thereof.

In certain embodiments, the viral expression construct of the present disclosure may be a plasmid vector or a baculoviral construct that encodes the parvoviral rep proteins for expression in insect cells. In certain embodiments, a single coding sequence is used for the Rep78 and Rep52 proteins, wherein start codon for translation of the Rep78 protein is a suboptimal start codon, selected from the group consisting of ACG, TTG, CTG, and GTG, that effects partial exon skipping upon expression in insect cells, as described in U.S. Pat. No. 8,512,981, the contents of which are herein incorporated by reference in their entirety, for example to promote less abundant expression of Rep78 as compared to Rep52, which may promote high vector yields.

In certain embodiments, a VP-coding region encodes one or more AAV capsid proteins of a specific AAV serotype. The AAV serotypes for VP-coding regions can be the same or different. In certain embodiments, a VP-coding region can be codon optimized. In certain embodiments, a VP-coding region or nucleotide sequence can be codon optimized for a mammal cell. In certain embodiments, a VP-coding region or nucleotide sequence can be codon optimized for an insect cell. In certain embodiments, a VP-coding region or nucleotide sequence can be codon optimized for a Spodoptera frugiperda cell. In certain embodiments, a VP-coding region or nucleotide sequence can be codon optimized for Sf9 or Sf21 cell lines.

In certain embodiments, a nucleotide sequence encoding one or more VP capsid proteins can be codon optimized to have a nucleotide homology with the reference nucleotide sequence of less than 100%. In certain embodiments, the nucleotide homology between the codon-optimized VP nucleotide sequence and the reference VP nucleotide sequence is less than 100%, less than 99%, less than 98%, less than 97%, less than 96%, less than 95%, less than 94%, less than 93%, less than 92%, less than 91%, less than 90%, less than 89%, less than 88%, less than 87%, less than 86%, less than 85%, less than 84%, less than 83%, less than 82%, less than 81%, less than 80%, less than 78%, less than 76%, less than 74%, less than 72%, less than 70%, less than 68%, less than 66%, less than 64%, less than 62%, less than 60%, less than 55%, less than 50%, and less than 40%.

In certain embodiments, a viral expression construct or a payload construct of the present disclosure can be a bacmid, also known as a baculovirus plasmid or recombinant baculovirus genome. In certain embodiments, a viral expression construct or a payload construct of the present disclosure (e.g. bacmid) can include a polynucleotide incorporated by homologous recombination (transposon donor/acceptor system) into the bacmid by standard molecular biology techniques known and performed by a person skilled in the art.

In certain embodiments, the polynucleotide incorporated into the bacmid (i.e. polynucleotide insert) can include an expression control sequence operably linked to a protein-coding nucleotide sequence. In certain embodiments, the polynucleotide incorporated into the bacmid can include an expression control sequence which includes a promoter, such as p10 or polh, and which is operably linked to a nucleotide sequence which encodes a structural AAV capsid protein (e.g. VP1, VP2, VP3 or a combination thereof). In certain embodiments, the polynucleotide incorporated into the bacmid can include an expression control sequence which includes a promoter, such as p10 or polh, and which is operably linked to a nucleotide sequence which encodes a non-structural AAV capsid protein (e.g. Rep78, Rep52, or a combination thereof).

The method of the present disclosure is not limited by the use of specific expression control sequences. However, when a certain stoichiometry of VP products are achieved (close to 1:1:10 for VP1, VP2, and VP3, respectively) and also when the levels of Rep52 or Rep40 (also referred to as the p19 Reps) are significantly higher than Rep78 or Rep68 (also referred to as the p5 Reps), improved yields of AAV in production cells (such as insect cells) may be obtained. In certain embodiments, the p5/p19 ratio is below 0.6 more, below 0.4, or below 0.3, but always at least 0.03. These ratios can be measured at the level of the protein or can be implicated from the relative levels of specific mRNAs.

In certain embodiments, AAV particles are produced in viral production cells (such as mammalian or insect cells) wherein all three VP proteins are expressed at a stoichiometry approaching, about or which is: 1:1:10 (VP1:VP2:VP3); 2:2:10 (VP1:VP2:VP3); 2:0:10 (VP1:VP2:VP3); 1-2:0-2:10 (VP1:VP2:VP3); 1-2:1-2:10 (VP1:VP2:VP3); 2-3:0-3:10 (VP1:VP2:VP3); 2-3:2-3:10 (VP1:VP2:VP3); 3:3:10 (VP1:VP2:VP3); 3-5:0-5:10 (VP1:VP2:VP3); or 3-5:3-5:10 (VP1:VP2:VP3).

In certain embodiments, the expression control regions are engineered to produce a VP1:VP2:VP3 ratio selected from the group consisting of: about or exactly 1:0:10; about or exactly 1:1:10; about or exactly 2:1:10; about or exactly 2:1:10; about or exactly 2:2:10; about or exactly 3:0:10; about or exactly 3:1:10; about or exactly 3:2:10; about or exactly 3:3:10; about or exactly 4:0:10; about or exactly 4:1:10; about or exactly 4:2:10; about or exactly 4:3:10; about or exactly 4:4:10; about or exactly 5:5:10; about or exactly 1-2:0-2:10; about or exactly 1-2:1-2:10; about or exactly 1-3:0-3:10; about or exactly 1-3:1-3:10; about or exactly 1-4:0-4:10; about or exactly 1-4:1-4:10; about or exactly 1-5:1-5:10; about or exactly 2-3:0-3:10; about or exactly 2-3:2-3:10; about or exactly 2-4:2-4:10; about or exactly 2-5:2-5:10; about or exactly 3-4:3-4:10; about or exactly 3-5:3-5:10; and about or exactly 4-5:4-5:10.

In certain embodiments of the present disclosure, Rep52 or Rep78 is transcribed from the baculoviral derived polyhedron promoter (polh). Rep52 or Rep78 can also be transcribed from a weaker promoter, for example a deletion mutant of the ie-1 promoter, the Δie-1 promoter, has about 20% of the transcriptional activity of that ie-1 promoter. A promoter substantially homologous to the Δie-1 promoter may be used. In respect to promoters, a homology of at least 50%, 60%, 70%, 80%, 90% or more, is considered to be a substantially homologous promoter.

### Mammalian Cells

Viral production of the present disclosure disclosed herein describes processes and methods for producing AAV particles or viral vector that contacts a target cell to deliver a payload construct, e.g. a recombinant AAV particle or viral construct, which includes a nucleotide encoding a payload molecule. The viral production cell may be selected from any biological organism, including prokaryotic (e.g., bacterial) cells, and eukaryotic cells, including, insect cells, yeast cells and mammalian cells.

In certain embodiments, the AAV particles of the present disclosure may be produced in a viral production cell that includes a mammalian cell. Viral production cells may comprise mammalian cells such as A549, WEH1, 3T3, 10T1/2, BHK, MDCK, COS 1, COS 7, BSC 1, BSC 40, BMT 10, VERO, W138, HeLa, HEK293, HEK293T (293T), Saos, C2C12, L cells, HT1080, Huh7, HepG2, C127, 3T3, CHO, HeLa cells, KB cells, BHK and primary fibroblast, hepatocyte, and myoblast cells derived from mammals. Viral production cells can include cells derived from any mammalian species including, but not limited to, human, monkey, mouse, rat, rabbit, and hamster or cell type, including but not limited to fibroblast, hepatocyte, tumor cell, cell line transformed cell, etc.

AAV viral production cells commonly used for production of recombinant AAV particles include, but is not limited to other mammalian cell lines as described in U.S. Pat. Nos. 6,156,303, 5,387,484, 5,741,683, 5,691,176, 6,428,988 and 5,688,676; U.S. patent application 2002/0081721, and International Patent Publication Nos. WO 00/47757, WO 00/24916, and WO 96/17947, the contents of each of which are herein incorporated by reference in their entireties insofar as they do no conflict with the present disclosure. In certain embodiments, the AAV viral production cells are trans-complementing packaging cell lines that provide functions deleted from a replication-defective helper virus, e.g., HEK293 cells or other Ea trans-complementing cells.

In certain embodiments, the packaging cell line 293-10-3 (ATCC Accession No. PTA-2361) may be used to produce the AAV particles, as described in U.S. Pat. No. 6,281,010, the contents of which are herein incorporated by reference in their entirety as related to the 293-10-3 packaging cell line and uses thereof.

In certain embodiments, of the present disclosure a cell line, such as a HeLA cell line, for trans-complementing E1 deleted adenoviral vectors, which encoding adenovirus E1a and adenovirus E1b under the control of a phosphoglycerate kinase (PGK) promoter can be used for AAV particle production as described in U.S. Pat. No. 6,365,394, the contents of which are incorporated herein by reference in their entirety as related to the HeLa cell line and uses thereof.

In certain embodiments, AAV particles are produced in mammalian cells using a multiplasmid transient transfection method (such as triple plasmid transient transfection). In certain embodiments, the multiplasmid transient transfection method includes transfection of the following three different constructs: (i) a payload construct, (ii) a Rep/Cap construct (parvoviral Rep and parvoviral Cap), and (iii) a helper construct. In certain embodiments, the triple transfection method of the three components of AAV particle production may be utilized to produce small lots of virus for assays including transduction efficiency, target tissue (tropism) evaluation, and stability. In certain embodiments, the triple transfection method of the three components of AAV particle production may be utilized to produce large lots of materials for clinical or commercial applications.

AAV particles to be formulated may be produced by triple transfection or baculovirus mediated virus production, or any other method known in the art. Any suitable permissive or packaging cell known in the art may be employed to produce the vectors. In certain embodiments, trans-complementing packaging cell lines are used that provide functions deleted from a replication-defective helper virus, e.g., 293 cells or other E1a trans-complementing cells.

The gene cassette may contain some or all of the parvovirus (e.g., AAV) cap and rep genes. In certain embodiments, some or all of the cap and rep functions are provided in trans by introducing a packaging vector(s) encoding the capsid and/or Rep proteins into the cell. In certain embodiments, the gene cassette does not encode the capsid or Rep proteins. Alternatively, a packaging cell line is used that is stably transformed to express the cap and/or rep genes.

Recombinant AAV virus particles are, in certain embodiments, produced and purified from culture supernatants according to the procedure as described in US2016/0032254, the contents of which are incorporated by reference in their entirety as related to the production and processing of recombinant AAV virus particles. Production may also involve methods known in the art including those using 293T cells, triple transfection or any suitable production method.

In certain embodiments, mammalian viral production cells (e.g. 293T cells) can be in an adhesion/adherent state (e.g. with calcium phosphate) or a suspension state (e.g. with polyethyleneimine (PEI)). The mammalian viral production cell is transfected with plasmids required for production of AAV, (i.e., AAV rep/cap construct, an adenoviral helper construct, and/or ITR flanked payload construct). In certain embodiments, the transfection process can include optional medium changes (e.g. medium changes for cells in adhesion form, no medium changes for cells in suspension form, medium changes for cells in suspension form if desired). In certain embodiments, the transfection process can include transfection mediums such as DMEM or F17. In certain embodiments, the transfection medium can include serum or can be serum-free (e.g. cells in adhesion state with calcium phosphate and with serum, cells in suspension state with PEI and without serum).

Cells can subsequently be collected by scraping (adherent form) and/or pelleting (suspension form and scraped adherent form) and transferred into a receptacle. Collection steps can be repeated as necessary for full collection of produced cells. Next, cell lysis can be achieved by consecutive freeze-thaw cycles (−80 C to 37 C), chemical lysis (such as adding detergent triton), mechanical lysis, or by allowing the cell culture to degrade after reaching ˜0% viability. Cellular debris is removed by centrifugation and/or depth filtration. The samples are quantified for AAV particles by DNase resistant genome titration by DNA qPCR.

AAV particle titers are measured according to genome copy number (genome particles per milliliter). Genome particle concentrations are based on DNA qPCR of the vector DNA as previously reported (Clark et al. (1999) Hum. Gene Ther., 10:1031-1039; Veldwijk et al. (2002) Mol. Ther., 6:272-278, the contents of which are each incorporated by reference in their entireties as related to the measurement of particle concentrations).

### Insect Cells

Viral production of the present disclosure includes processes and methods for producing AAV particles or viral vectors that contact a target cell to deliver a payload construct, e.g., a recombinant viral construct, which includes a nucleotide encoding a payload molecule. In certain embodiments, the AAV particles or viral vectors of the present disclosure may be produced in a viral production cell that includes an insect cell.

Growing conditions for insect cells in culture, and production of heterologous products in insect cells in culture are well-known in the art, see U.S. Pat. No. 6,204,059, the contents of which are herein incorporated by reference in their entirety as related to the growth and use of insect cells in viral production.

Any insect cell which allows for replication of parvovirus and which can be maintained in culture can be used in accordance with the present disclosure. AAV viral production cells commonly used for production of recombinant AAV particles include, but is not limited to, Spodoptera frugiperda, including, but not limited to the Sf9 or Sf21 cell lines, Drosophila cell lines, or mosquito cell lines, such as Aedes albopictus derived cell lines. Use of insect cells for expression of heterologous proteins is well documented, as are methods of introducing nucleic acids, such as vectors, e.g., insect-cell compatible vectors, into such cells and methods of maintaining such cells in culture. See, for example, Methods in Molecular Biology, ed. Richard, Humana Press, NJ (1995); O'Reilly et al., Baculovirus Expression Vectors, A Laboratory Manual, Oxford Univ. Press (1994); Samulski et al., J. Vir. 63:3822-8 (1989); Kajigaya et al., Proc. Nat'l. Acad. Sci. USA 88: 4646-50 (1991); Ruffing et al., J. Vir. 66:6922-30 (1992); Kimbauer et al., Vir. 219:37-44 (1996); Zhao et al., Vir. 272:382-93 (2000); and Samulski et al., U.S. Pat. No. 6,204,059, the contents of each of which are herein incorporated by reference in their entirety as related to the use of insect cells in viral production.

In some embodiments, the AAV particles are made using the methods described in WO2015/191508, the contents of which are herein incorporated by reference in their entirety insofar as they do not conflict with the present disclosure.

In certain embodiments, insect host cell systems, in combination with baculoviral systems (e.g., as described by Luckow et al., Bio/Technology 6: 47 (1988)) may be used. In certain embodiments, an expression system for preparing chimeric peptide is Trichoplusia ni, Tn 5B1-4 insect cells/baculoviral system, which can be used for high levels of proteins, as described in U.S. Pat. No. 6,660,521, the contents of which are herein incorporated by reference in their entirety as related to the production of viral particles.

Expansion, culturing, transfection, infection and storage of insect cells can be carried out in any cell culture media, cell transfection media or storage media known in the art, including Hyclone™ SFX-Insect™ Cell Culture Media, Expression System ESF AF™ Insect Cell Culture Medium, ThermoFisher Sf-900II™ media, ThermoFisher Sf-900III™ media, or ThermoFisher Grace's Insect Media. Insect cell mixtures of the present disclosure can also include any of the formulation additives or elements described in the present disclosure, including (but not limited to) salts, acids, bases, buffers, surfactants (such as Poloxamer 188/Pluronic F-68), and other known culture media elements. Formulation additives can be incorporated gradually or as “spikes” (incorporation of large volumes in a short time).

### Baculovirus-Production Systems

In certain embodiments, processes of the present disclosure can include production of AAV particles or viral vectors in a baculoviral system using a viral expression construct and a payload construct vector. In certain embodiments, the baculoviral system includes Baculovirus expression vectors (BEVs) and/or baculovirus infected insect cells (BIICs). In certain embodiments, a viral expression construct or a payload construct of the present disclosure can be a bacmid, also known as a baculovirus plasmid or recombinant baculovirus genome. In certain embodiments, a viral expression construct or a payload construct of the present disclosure can be polynucleotide incorporated by homologous recombination (transposon donor/acceptor system) into a bacmid by standard molecular biology techniques known and performed by a person skilled in the art. Transfection of separate viral replication cell populations produces two or more groups (e.g. two, three) of baculoviruses (BEVs), one or more group which can include the viral expression construct (Expression BEV), and one or more group which can include the payload construct (Payload BEV). The baculoviruses may be used to infect a viral production cell for production of AAV particles or viral vector.

In certain embodiments, the process includes transfection of a single viral replication cell population to produce a single baculovirus (BEV) group which includes both the viral expression construct and the payload construct. These baculoviruses may be used to infect a viral production cell for production of AAV particles or viral vector.

In certain embodiments, BEVs are produced using a Bacmid Transfection agent, such as Promega FuGENE® HD, WFI water, or ThermoFisher Cellfectin® II Reagent. In certain embodiments, BEVs are produced and expanded in viral production cells, such as an insect cell.

In certain embodiments, the method utilizes seed cultures of viral production cells that include one or more BEVs, including baculovirus infected insect cells (BIICs). The seed BIICs have been transfected/transduced/infected with an Expression BEV which includes a viral expression construct, and also a Payload BEV which includes a payload construct. In certain embodiments, the seed cultures are harvested, divided into aliquots and frozen, and may be used at a later time to initiate transfection/transduction/infection of a naïve population of production cells. In certain embodiments, a bank of seed BIICs is stored at −80° C. or in LN2 vapor.

Baculoviruses are made of several essential proteins which are essential for the function and replication of the Baculovirus, such as replication proteins, envelope proteins and capsid proteins. The Baculovirus genome thus includes several essential-gene nucleotide sequences encoding the essential proteins. As a non-limiting example, the genome can include an essential-gene region which includes an essential-gene nucleotide sequence encoding an essential protein for the Baculovirus construct. The essential protein can include: GP64 baculovirus envelope protein, VP39 baculovirus capsid protein, or other similar essential proteins for the Baculovirus construct.

Baculovirus expression vectors (BEV) for producing AAV particles in insect cells, including but not limited to Spodoptera frugiperda (Sf9) cells, provide high titers of viral vector product. Recombinant baculovirus encoding the viral expression construct and payload construct initiates a productive infection of viral vector replicating cells. Infectious baculovirus particles released from the primary infection secondarily infect additional cells in the culture, exponentially infecting the entire cell culture population in a number of infection cycles that is a function of the initial multiplicity of infection, see Urabe, M. et al. J Virol. 2006 February; 80(4):1874-85, the contents of which are herein incorporated by reference in their entirety as related to the production and use of BEVs and viral particles.

Production of AAV particles with baculovirus in an insect cell system may address known baculovirus genetic and physical instability.

In certain embodiments, the production system of the present disclosure addresses baculovirus instability over multiple passages by utilizing a titerless infected-cells preservation and scale-up system. Small scale seed cultures of viral producing cells are transfected with viral expression constructs encoding the structural and/or non-structural components of the AAV particles. Baculovirus-infected viral producing cells are harvested into aliquots that may be cryopreserved in liquid nitrogen; the aliquots retain viability and infectivity for infection of large scale viral producing cell culture. Wasilko D J et al. Protein Expr Purif. 2009 June; 65(2):122-32, the contents of which are herein incorporated by reference in their entirety as related to the production and use of BEVs and viral particles.

A genetically stable baculovirus may be used to produce a source of the one or more of the components for producing AAV particles in invertebrate cells. In certain embodiments, defective baculovirus expression vectors may be maintained episomally in insect cells. In such embodiments, the corresponding bacmid vector is engineered with replication control elements, including but not limited to promoters, enhancers, and/or cell-cycle regulated replication elements.

In certain embodiments, stable viral producing cells permissive for baculovirus infection are engineered with at least one stable integrated copy of any of the elements necessary for AAV replication and vector production including, but not limited to, the entire AAV genome, Rep and Cap genes, Rep genes, Cap genes, each Rep protein as a separate transcription cassette, each VP protein as a separate transcription cassette, the AAP (assembly activation protein), or at least one of the baculovirus helper genes with native or non-native promoters.

In some embodiments, the AAV particle of the present disclosure may be produced in insect cells (e.g., Sf9 cells).

In some embodiments, the AAV particle of the present disclosure may be produced using triple transfection.

In some embodiments, the AAV particle of the present disclosure may be produced in mammalian cells.

In some embodiments, the AAV particle of the present disclosure may be produced by triple transfection in mammalian cells.

In some embodiments, the AAV particle of the present disclosure may be produced by triple transfection in HEK293 cells.

The AAV viral genomes encoding an anti-tau antibody molecule described herein may be useful in the fields of human disease, veterinary applications and a variety of in vivo and in vitro settings. The AAV particles of the present disclosure may be useful in the field of medicine for the treatment, prophylaxis, palliation, or amelioration of neurological or neuromuscular diseases and/or disorders. In some embodiments, the AAV particles of the disclosure are used for the prevention and/or treatment of tau-related disorders.

Various embodiments of the disclosure herein provide a pharmaceutical composition comprising the AAV particle described herein and a pharmaceutically acceptable excipient.

Various embodiments of the disclosure herein provide a method of treating a subject in need thereof comprising administering to the subject a therapeutically effective amount of the pharmaceutical composition described herein.

Certain embodiments of the method provide that the subject is treated by a route of administration of the pharmaceutical composition selected from the group consisting of: intravenous, intracerebroventricular, intraparenchymal, intrathecal, subpial, and intramuscular, or a combination thereof. Certain embodiments of the method provide that the subject is treated for tau-related disorders and/or other neurological disorder arising from a deficiency in the quantity or function of tau gene products. In one aspect of the method, a pathological feature of the tau-related disorders or the other neurological disorder is alleviated and/or the progression of the tau-related disorders or the other neurological disorder is halted, slowed, ameliorated, or reversed.

Also described herein are compositions, methods, processes, kits and devices for the design, preparation, manufacture and/or formulation of AAV particles. In some embodiments, payloads, such as but not limited to payloads comprising an anti-tau antibody molecule, may be encoded by payload constructs or contained within plasmids or vectors or recombinant adeno-associated viruses (AAVs).

The present disclosure also provides administration and/or delivery methods for vectors and viral particles, e.g., AAV particles, for the treatment or amelioration of tau-related disorders. Such methods may involve gene replacement or gene activation. Such outcomes are achieved by utilizing the methods and compositions taught herein.

### III. Pharmaceutical Compositions

The present disclosure additionally provides a method for treating tau-related disorders and disorders related to the abnormal function or expression of tau in a mammalian subject, including a human subject, comprising administering to the subject any of the AAV polynucleotides or AAV genomes described herein (i.e., “vector genomes,” “viral genomes,” or “VGs”) or administering to the subject a particle comprising said AAV polynucleotide or AAV genome, or administering to the subject any of the described compositions, including pharmaceutical compositions.

The AAV particle described herein may be prepared as a pharmaceutical composition. In some embodiments, the pharmaceutical composition comprises at least one active ingredients. In some embodiments, the pharmaceutical composition comprises a pharmaceutically acceptable excipient.

Although the descriptions of pharmaceutical compositions, e.g., AAV comprising a payload encoding an anti-tau antibody molecule to be delivered, provided herein are principally directed to pharmaceutical compositions which are suitable for administration to humans, it will be understood by the skilled artisan that such compositions are generally suitable for administration to any other animal, e.g., to non-human animals, e.g. non-human mammals. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals such as cattle, pigs, horses, sheep, cats, dogs, mice, and/or rats; and/or birds, including commercially relevant birds such as poultry, chickens, ducks, geese, and/or turkeys.

In some embodiments, compositions are administered to humans, human patients, or subjects.

In some embodiments, the AAV particle formulations described herein may contain a nucleic acid encoding at least one payload. In some embodiments, the formulations may contain a nucleic acid encoding 1, 2, 3, 4, or 5 payloads. In some embodiments, the formulation may contain a nucleic acid encoding a payload construct encoding proteins selected from categories such as, but not limited to, human proteins, veterinary proteins, bacterial proteins, biological proteins, antibodies, immunogenic proteins, therapeutic peptides and proteins, secreted proteins, plasma membrane proteins, cytoplasmic proteins, cytoskeletal proteins, intracellular membrane bound proteins, nuclear proteins, proteins associated with human disease, and/or proteins associated with non-human diseases. In some embodiments, the formulation contains at least three payload constructs encoding proteins. Certain embodiments provide that at least one of the payloads is an anti-tau antibody molecule.

A pharmaceutical composition in accordance with the present disclosure may be prepared, packaged, and/or sold in bulk, as a single unit dose, and/or as a plurality of single unit doses. As used herein, a “unit dose” refers to a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

### IV. Formulations

Formulations of the AAV pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product into a desired single- or multi-dose unit.

Relative amounts of the active ingredient, the pharmaceutically acceptable excipient, and/or any additional ingredients in a pharmaceutical composition in accordance with the disclosure will vary, depending upon the identity, size, and/or condition of the subject treated and further depending upon the route by which the composition is to be administered.

For example, the composition may comprise between 0.1% and 99% (w/w) of the active ingredient. By way of example, the composition may comprise between 0.1% and 100%, e.g., between 0.5% and 50%, between 1-30%, between 5-80%, or at least 80% (w/w) active ingredient.

The AAV particles of the disclosure can be formulated using one or more excipients to: (1) increase stability; (2) increase cell transfection or transduction; (3) permit the sustained or delayed release; (4) alter the biodistribution (e.g., target the viral particle to specific tissues or cell types); (5) increase the translation of encoded protein in vivo; (6) alter the release profile of encoded protein in vivo and/or (7) allow for regulatable expression of the payload.

Formulations of the present disclosure can include, without limitation, saline, lipidoids, liposomes, lipid nanoparticles, polymers, lipoplexes, core-shell nanoparticles, peptides, proteins, cells transfected with viral vectors (e.g., for transplantation into a subject), nanoparticle mimics and combinations thereof. Further, the viral vectors of the present disclosure may be formulated using self-assembled nucleic acid nanoparticles.

In some embodiments, the viral vectors encoding an anti-tau antibody molecule may be formulated to optimize baricity and/or osmolality. In some embodiments, the baricity and/or osmolality of the formulation may be optimized to ensure optimal drug distribution in the central nervous system or a region or component of the central nervous system.

In some embodiments, the AAV particles of the disclosure may be formulated in PBS with 0.001% of pluronic acid (F-68) at a pH of about 7.0.

In some embodiments, the AAV particles of the disclosure may be formulated in PBS, in combination with an ethylene oxide/propylene oxide copolymer (also known as pluronic or poloxamer).

In some embodiments, the AAV particles of the disclosure may be formulated in PBS with 0.001% pluronic acid (F-68) (poloxamer 188) at a pH of about 7.0.

In some embodiments, the AAV particles of the disclosure may be formulated in PBS with 0.001% pluronic acid (F-68) (poloxamer 188) at a pH of about 7.3.

In some embodiments, the AAV particles of the disclosure may be formulated in PBS with 0.001% pluronic acid (F-68) (poloxamer 188) at a pH of about 7.4.

In some embodiments, the AAV particles of the disclosure may be formulated in a solution comprising sodium chloride, sodium phosphate and an ethylene oxide/propylene oxide copolymer.

In some embodiments, the AAV particles of the disclosure may be formulated in a solution comprising sodium chloride, sodium phosphate dibasic, potassium chloride, potassium phosphate monobasic, and poloxamer 188/pluronic acid (F-68).

In some embodiments, the AAV particles of the disclosure may be formulated in a solution comprising 192 mM sodium chloride, 10 mM sodium phosphate (dibasic), 2.7 mM potassium chloride, 2 mM potassium phosphate (monobasic) and 0.001% pluronic F-68 (v/v), at pH 7.4. This formulation is referred to as Formulation 1 in the present disclosure.

In some embodiments, the AAV particles of the disclosure may be formulated in a solution comprising about 192 mM sodium chloride, about 10 mM sodium phosphate dibasic and about 0.001% poloxamer 188, at a pH of about 7.3. The concentration of sodium chloride in the final solution may be 150 mM-200 mM. As non-limiting examples, the concentration of sodium chloride in the final solution may be 150 mM, 160 mM, 170 mM, 180 mM, 190 mM or 200 mM. The concentration of sodium phosphate dibasic in the final solution may be 1 mM-50 mM. As non-limiting examples, the concentration of sodium phosphate dibasic in the final solution may be 1 mM, 2 mM, 3 mM, 4 mM, 5 mM, 6 mM, 7 mM, 8 mM, 9 mM, 10 mM, 15 mM, 20 mM, 25 mM, 30 mM, 40 mM, or 50 mM. The concentration of poloxamer 188 (pluronic acid (F-68)) may be 0.0001%-1%. As non-limiting examples, the concentration of poloxamer 188 (pluronic acid (F-68)) may be 0.0001%, 0.0005%, 0.001%, 0.005%, 0.01%, 0.05%, 0.1%, 0.5%, or 1%. The final solution may have a pH of 6.8-7.7. Non-limiting examples for the pH of the final solution include a pH of 6.8, 6.9, 7.0, 7.1, 7.2, 7.3, 7.4, 7.5, 7.6, or 7.7.

In some embodiments, the AAV particles of the disclosure may be formulated in a solution comprising about 1.05% sodium chloride, about 0.212% sodium phosphate dibasic, heptahydrate, about 0.025% sodium phosphate monobasic, monohydrate, and 0.001% poloxamer 188, at a pH of about 7.4. As a non-limiting example, the concentration of AAV particle in this formulated solution may be about 0.001%. The concentration of sodium chloride in the final solution may be 0.1-2.0%, with non-limiting examples of 0.1%, 0.25%, 0.5%, 0.75%, 0.95%, 0.96%, 0.97%, 0.98%, 0.99%, 1.00%, 1.01%, 1.02%, 1.03%, 1.04%, 1.05%, 1.06%, 1.07%, 1.08%, 1.09%, 1.10%, 1.25%, 1.5%, 1.75%, or 2%. The concentration of sodium phosphate dibasic in the final solution may be 0.100-0.300% with non-limiting examples including 0.100%, 0.125%, 0.150%, 0.175%, 0.200%, 0.210%, 0.211%, 0.212%, 0.213%, 0.214%, 0.215%, 0.225%, 0.250%, 0.275%, 0.300%. The concentration of sodium phosphate monobasic in the final solution may be 0.010-0.050%, with non-limiting examples of 0.010%, 0.015%, 0.020%, 0.021%, 0.022%, 0.023%, 0.024%, 0.025%, 0.026%, 0.027%, 0.028%, 0.029%, 0.030%, 0.035%, 0.040%, 0.045%, or 0.050%. The concentration of poloxamer 188 (pluronic acid (F-68)) may be 0.0001%-1%. As non-limiting examples, the concentration of poloxamer 188 (pluronic acid (F-68)) may be 0.0001%, 0.0005%, 0.001%, 0.005%, 0.01%, 0.05%, 0.1%, 0.5%, or 1%. The final solution may have a pH of 6.8-7.7. Non-limiting examples for the pH of the final solution include a pH of 6.8, 6.9, 7.0, 7.1, 7.2, 7.3, 7.4, 7.5, 7.6, or 7.7.

In some embodiments, a formulation comprising an AAV particle described herein comprises a sufficient, e.g., effective amount, of AAV particles for expression of an antibody molecule, e.g., an antibody molecule that binds to tau. In some embodiments, an AAV particle comprises a genetic element encoding at least 1, 2, 3, 4, or 5 antibody molecules.

The present disclosure provides, in some embodiments, an AAV particle formulated for delivery to the central nervous system (CNS). In some embodiments, an agent that crosses the brain blood barrier may be used. For example, some cell penetrating peptides that can target molecules to the brain blood barrier endothelium may be used for formulation (e.g., Mathupala, Expert Opin Ther Pat., 2009, 19, 137-140; the content of which is incorporated herein by reference in its entirety).

### Excipients

The formulations of the disclosure can include one or more excipients, each in an amount that together increases the stability of the AAV particle, increases cell transfection or transduction by the viral particle, increases the expression of viral particle encoded protein, and/or alters the release profile of AAV particle encoded proteins. In some embodiments, a pharmaceutically acceptable excipient may be at least 95%, at least 96%, at least 97%, at least 98%, at least 99%, or 100% pure. In some embodiments, an excipient is approved for use for humans and for veterinary use. In some embodiments, an excipient may be approved by United States Food and Drug Administration. In some embodiments, an excipient may be of pharmaceutical grade. In some embodiments, an excipient may meet the standards of the United States Pharmacopoeia (USP), the European Pharmacopoeia (EP), the British Pharmacopoeia, and/or the International Pharmacopoeia.

Excipients, which, as used herein, include, but are not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired. Various excipients for formulating pharmaceutical compositions and techniques for preparing the composition are known in the art (see Remington: The Science and Practice of Pharmacy, 21st Edition, A. R. Gennaro, Lippincott, Williams & Wilkins, Baltimore, MD, 2006; the contents of which are herein incorporated by reference in their entirety). The use of a conventional excipient medium may be contemplated within the scope of the present disclosure, except insofar as any conventional excipient medium may be incompatible with a substance or its derivatives, such as by producing any undesirable biological effect or otherwise interacting in a deleterious manner with any other component(s) of the pharmaceutical composition.

### Inactive Ingredients

In some embodiments, AAV formulations may comprise at least one excipient which is an inactive ingredient. As used herein, the term “inactive ingredient” refers to one or more agents that do not contribute to the activity of the pharmaceutical composition included in formulations. In some embodiments, all, none, or some of the inactive ingredients which may be used in the formulations of the present disclosure may be approved by the US Food and Drug Administration (FDA).

Formulations of AAV particles disclosed herein may include cations or anions. In some embodiments, the formulations include metal cations such as, but not limited to, Zn²⁺, Ca²⁺, Cu²⁺, Mg⁺, or combinations thereof. In some embodiments, formulations may include polymers or polynucleotides complexed with a metal cation (see, e.g., U.S. Pat. Nos. 6,265,389 and 6,555,525, the contents of each of which are herein incorporated by reference in their entirety).

### IV. Methods and Uses of the Compositions

The present disclosure provides a method for treating a disease, disorder and/or condition in a mammalian subject, including a human subject, comprising administering to the subject any of the AAV particles described herein or administering to the subject any of the described compositions, including pharmaceutical compositions, described herein.

In some embodiments, the AAV particles of the present disclosure are administered to a subject prophylactically.

In some embodiments, the AAV particles of the present disclosure are administered to a subject having at least one of the diseases described herein.

In some embodiments, the AAV particles of the present disclosure are administered to a subject to treat a disease or disorder described herein. The subject may have the disease or disorder or may be at-risk to developing the disease or disorder.

In some embodiments, the AAV particles of the present disclosure are part of an active immunization strategy to protect against diseases and disorders. In an active immunization strategy, a vaccine or AAV particles are administered to a subject to prevent an infectious disease by activating the subject's production of antibodies that can fight off invading bacteria or viruses.

In some embodiments, the AAV particles of the present disclosure are part of a passive immunization strategy. In a passive immunization strategy, antibodies against a particular infectious agent are given directly to the subject.

In some embodiments, the AAV particles of the present disclosure may be used for passive immunotherapy of tauopathy, e.g., Alzheimer Disease or Frontotemporal Dementia, as described in Liu et al, the contents of which are herein incorporated by reference in their entirety (Liu, W et al., 2016 J Neurosci 36(49):12425-12435).

In some embodiments, the AAV particles of the present disclosure may be used to treat tauopathy as described in Ising et al, the contents of which are herein incorporated by reference in their entirety (Ising, C et al., 2017 J Exp Med April 17, Epub ahead of print). Treatment with the AAV particles may result in a significant reduction in neuropathological tau species in the hippocampus.

Passive immunization by intravenous (or intraperitoneal in mice) delivery of antibody has been shown to result in substantial serum levels of antibody (Chai et al., 2011, J Biol Chem., 286, 34457-34467, the contents of which are herein incorporated by reference in their entirety) and reduced tau pathology in mouse models of tauopathy (e.g., P301L or P301S mice). However, these reductions in tau pathology are considered modest (e.g., about 34%) and require high and repeated dosing to achieve. Passive immunization strategies are thought to be limited in their ability to deliver large quantity of antibody to the brain, which may limit efficacy, and are also challenged in addressing intracellular aggregates. In some embodiments, delivery of an AAV particle comprising a viral genome encoding an anti-tau antibody molecule can be used to overcome the limitations seen with passive immunization strategies.

In some embodiments, the administration of AAV particles of the present disclosure may result in substantially higher antibody levels in the target tissue (e.g., CNS) of the subject than if anti-tau antibodies were administered by passive immunization. In some embodiments, AAV mediated delivery can result in 1.5-16 fold higher antibody levels in the brain than if delivered by passive immunization. Whilst not wishing to be bound by theory, passive immunization is thought to generate 20-40 ng of antibody per mg of protein in the brain of the subject. In some embodiments, AAV-mediated delivery results in antibody levels 2-5× above the levels seen with passive immunization. In some embodiments, AAV-mediated delivery results in antibody levels 1.5-3× above the levels seen with passive immunization. In some embodiments, AAV-mediated delivery results in antibody levels 5-10× above the levels seen with passive immunization. In some embodiments, AAV-mediated delivery results in antibody levels 8-16× above the levels seen with passive immunization. In some embodiments, the AAV particles are administered intravenously.

In some embodiments, AAV mediated delivery of anti-tau antibody molecule may be used to reduce tau seeding, prevent tau seeding and/or prevent the propagation of tau seeds in a subject. Tau may exist in both a monomeric form and in different aggregated forms. As used herein, the term “tau aggregate” refers to a molecular complex that comprises two or more tau monomers. A tau aggregate may include a nearly unlimited number of monomers bound together. For example, a tau aggregate may comprise 2, 3, 4, 5, 6, 7, 8, 9, 10 or more tau monomers. Alternatively, a tau aggregate may include 20, 30, 40, 50, 60, 70, 80, 90, 100 or more tau monomers. A tau aggregate may also include 500, 1000, 1500, 2000, 2500, 3000, 3500, 4000, 4500, 5000 or more tau monomers. The terms “fibrillar tau aggregate” and “tau fibril” may refer to forms of tau aggregates, and these terms are used interchangeably herein. A fibrillar tau aggregate is a polymeric, ordered fiber comprising tau. Tau fibrils are generally not soluble, but shorter assemblies, or oligomers, can be soluble. Tau aggregate also refers to soluble tau oligomers and protofibrils, which may act as intermediates during tau aggregation. Also included in the definition of tau aggregate is the term “tau seed”, which refers to a tau aggregate that is capable of nucleating or “seeding” intracellular tau aggregation when internalized by a cell, or when exposed to monomeric tau in vitro.

Tau seeding activity may be assessed in vitro in cellular tau models or in vivo in mouse models. Tau seeds for efficacy studies may be variant Tau fibrils such as, but not limited to, htau40, P301S, P301L, and K18. Tau seeds may also be prepared from brain lysates of subjects with Alzheimer's disease and/or with a P301S mutation. Tau seeds may also include enriched PHFs (ePHF) or immuno purified PHF (iPHF).

In vivo, the accumulation of intracellular tau amyloids defines tauopathies. In early disease stages, the disease pathology is generally localized to discrete regions of the brain, but with disease progression, pathology invariably spreads along distinct neural networks. Accumulating evidence suggests transcellular propagation of tau seeds underlies this pathology. In this model, tau are released from donor cells and enter neighboring cells, transforming native tau protein into the misfolded form via templated conformational change. AAV particles or pharmaceutical formulations containing the AAV particles of the disclosure may be administered to a subject in order to prevent, delay or reduce the progression of tau seeds through neural networks.

### Diagnostic Applications

The AAV particles of the present disclosure may be used for diagnostic purposes or as diagnostic tools for any disease or disorder. As non-limiting examples, the AAV particles of the present disclosure or the antibody molecules encoded within the viral genome therein may be used as a biomarker for disease diagnosis. As a second non-limiting example, the AAV particles of the present disclosure or the antibody molecules encoded within the viral genome therein may be used for diagnostic imaging purposes, e.g., MRI, PET, CT or ultrasound.

### Preventative Applications

The AAV particles of the present disclosure or the antibody molecules encoded by the viral genome therein may be used to prevent disease or stabilize the progression of disease. In some embodiments, the AAV particles of the present disclosure are used to as a prophylactic to prevent a disease or disorder in the future. In some embodiments, the AAV particles of the present disclosure are used to halt further progression of a disease or disorder. As a non-limiting example, the AAV particles may be used in a manner similar to that of a vaccine.

### Research Applications

The AAV particles of the present disclosure or the antibody molecules encoded by the viral genome therein may also be used as research tools. The AAV particles may be used as in any research experiment, e.g., in vivo or in vitro experiments. In a non-limiting example, the AAV particles may be used in cultured cells. The cultured cells may be derived from any origin known to one with skill in the art, and may be as non-limiting examples, derived from a stable cell line, an animal model or a human patient or control subject. In a non-limiting example, the AAV particles may be used in in vivo experiments in animal models (i.e., mouse, rat, rabbit, dog, cat, non-human primate, guinea pig, ferret, c-elegans, drosophila, zebrafish, or any other animal used for research purposes, known in the art). In another non-limiting example, the AAV particles may be used in human research experiments or human clinical trials.

### Combination Applications

The AAV particles may be used as a combination therapy with any other therapeutic molecule known in the art. The therapeutic molecule may be approved by the US Food and Drug Administration or may be in clinical trial or at the preclinical research stage. The therapeutic molecule may utilize any therapeutic modality known in the art, with non-limiting examples including gene silencing or interference (i.e., miRNA, siRNA, RNAi, shRNA), gene editing (i.e., TALEN, CRISPR/Cas9 systems, zinc finger nucleases), and gene, protein or enzyme replacement.

### Therapeutic Applications

The present disclosure additionally provides a method for treating neurological diseases and/or disorders in a mammalian subject, including a human subject, comprising administering to the subject any of the AAV particles described herein. In some cases, neurological diseases and/or disorders treated according to methods described herein include indications involving irregular expression or aggregation of tau. Such indications may include, but are not limited to Alzheimer's disease (AD), frontotemporal dementia and parkinsonism linked to chromosome 17 (FTDP-17), Frontotemporal lobar degeneration (FTLD), Frontotemporal dementia (FTD), chronic traumatic encephalopathy (CTE), Progressive Supranuclear Palsy (PSP), Down's syndrome, Pick's disease, Corticobasal degeneration (CBD), Corticobasal syndrome, Amyotrophic lateral sclerosis (ALS), Prion diseases, Creutzfeldt-Jakob disease (CJD), Multiple system atrophy, Tangle-only dementia, and Progressive subcortical gliosis.

In some embodiments, methods of treating neurological diseases and/or disorders in a subject in need thereof may comprise the steps of: (1) deriving, generating and/or selecting an anti-tau antibody molecule, or antibody-based composition; (2) producing an AAV particle with a viral genome that includes a payload region encoding the selected antibody molecule of (1); and (3) administering the AAV particle (or pharmaceutical composition thereof) to the subject.

The present disclosure provides a method for administering to a subject in need thereof, including a human subject, a therapeutically effective amount of the AAV particles to slow, stop or reverse disease progression. As a non-limiting example, disease progression may be measured by cognitive tests such as, but not limited to, the Mini-Mental State Exam (MMSE) or other similar diagnostic tool(s), known to those skilled in the art. As another non-limiting example, disease progression may be measured by change in the pathological features of the brain, CSF or other tissues of the subject, such as, but not limited to a decrease in levels of tau (either soluble or insoluble). In some embodiments, the levels of insoluble hyperphosphorylated tau are decreased. In some embodiments levels of soluble tau are decreased. In some embodiments both soluble and insoluble tau are decreased. In some embodiments, levels of insoluble hyperphosphorylated tau are increased. In some embodiments levels of soluble tau are increased. In some embodiments both insoluble and soluble tau levels are increased. In some embodiments, neurofibrillary tangles are decreased in size, number, density, or combination thereof. In another embodiment, neurofibrillary tangles are increased in size, number, density or combination thereof.

### Alzheimer's Disease

Alzheimer Disease (AD) is a debilitating neurodegenerative disease currently afflicting more than 35 million people worldwide, with that number expected to double in coming decades. Symptomatic treatments have been available for many years but these treatments do not address the underlying pathophysiology. Recent clinical trials using these and other treatments have largely failed and, to date, no known cure has been identified.

The AD brain is characterized by the presence of two forms of pathological aggregates, the extracellular plaques composed of β-amyloid (AD) and the intracellular neurofibrillary tangles (NFT) comprised of hyperphosphorylated microtubule associated protein tau. Based on early genetic findings, β-amyloid alterations were thought to initiate disease, with changes in tau considered downstream. Thus, most clinical trials have been AD-centric. Although no mutations of the tau gene have been linked to AD, such alterations have been shown to result in a family of dementias known as tauopathies, demonstrating that changes in tau can contribute to neurodegenerative processes. Tau is normally a very soluble protein known to associate with microtubules based on the extent of its phosphorylation. Hyperphosphorylation of tau depresses its binding to microtubules and microtubule assembly activity. In tauopathies, the tau becomes hyperphosphorylated, misfolds and aggregates as NFT of paired helical filaments (PHF), twisted ribbons or straight filaments. In AD, NFT pathology, rather than plaque pathology, correlates more closely with neuropathological markers such as neuronal loss, synaptic deficits, severity of disease and cognitive decline. NFT pathology marches through the brain in a stereotyped manner and animal studies suggest a trans-cellular propagation mechanism along neuronal connections.

Several approaches have been proposed for therapeutically interfering with progression of tau pathology and preventing the subsequent molecular and cellular consequences. Given that NFT are composed of a hyperphosphorylated, misfolded and aggregated form of tau, interference at each of these stages has yielded the most avidly pursued set of targets. Introducing agents that limit phosphorylation, block misfolding or prevent aggregation have all generated promising results. Passive and active immunization with late stage anti-phospho-tau antibodies in mouse models has led to dramatic decreases in tau aggregation and improvements in cognitive parameters. It has also been suggested that introduction of anti-tau antibodies can prevent the trans-neuronal spread of tau pathology.

In some embodiments, administration of AAV particles which encode an anti-tau antibody molecule described herein may be used to treat subjects suffering from AD and other tauopathies. In some cases, methods of the present disclosure may be used to treat subjects suspected of developing AD or other tauopathies.

### Frontotemporal Dementia and Parkinsonism Linked to Chromosome 17 (FTDP-17)

Although Alzheimer's disease is, in part, characterized by the presence of tau pathology, no known mutations in the tau gene have been causally linked to the disease. Mutations in the tau gene have been shown to lead to an autosomal dominantly inherited tauopathy known as frontotemporal dementia and parkinsonism linked to chromosome 17 (FTDP-17) and demonstrate that alterations in tau can lead to neurodegenerative changes in the brain. Mutations in the tau gene that lead to FTDP-17 are thought to influence splicing patterns, thereby leading to an elevated proportion of tau with four microtubule binding domains (rather than three). These molecules are considered to be more amyloidogenic, meaning they are more likely to become hyperphosphorylated and more likely to aggregate into NFT (Hutton, M. et al., 1998, Nature 393(6686):702-5). Although physically and behaviorally, FTDP-17 patients can appear quite similar to Alzheimer's disease patients, at autopsy FTDP-17 brains lack the prominent Aβ plaque pathology of an AD brain (Gotz, J. et al., 2012, British Journal of Pharmacology 165(5):1246-59). Therapeutically targeting the aggregates of tau protein may ameliorate and prevent degenerative changes in the brain and potentially lead to improved cognitive ability.

As of today, there is no treatment to prevent, slow the progression, or cure FTDP-17. Medication may be prescribed to reduce aggressive, agitated or dangerous behavior. There remains a need for therapy affecting the underlying pathophysiology, such as antibody therapies targeting tau protein.

In some embodiments, administration of AAV particles which encode an anti-tau antibody molecule described herein may be used to treat subjects suffering from FTDP-17. In some cases, methods of the present disclosure may be used to treat subjects suspected of developing FTDP-17.

### Chronic Traumatic Encephalopathy

Unlike the genetically linked tauopathies, chronic traumatic encephalopathy is a degenerative tauopathy linked to repeated head injuries. The disease was first described in boxers who behaved “punch drunk” and has since been identified primarily in athletes that play American football, ice hockey, wrestling and other contact sports. The brains of those suffering from CTE are characterized by distinctive patterns of brain atrophy accompanied by accumulation of hyperphosphorylated species of aggregated tau in NFT. In CTE, pathological changes in tau are accompanied by a number of other pathobiological processes, such as inflammation (Daneshvar, D. H. et al., 2015 Mol Cell Neurosci 66 (Pt B): 81-90). Targeting the tau aggregates may provide reprieve from the progression of the disease and may allow cognitive improvement.

As of today, there is no medical therapy to treat or cure CTE. The condition is only diagnosed after death, due to lack of in vivo techniques to identify CTE specific biomarkers. There remains a need for therapy affecting the underlying pathophysiology, such as antibody therapies targeting tau protein.

In some embodiments, administration of AAV particles which encode an anti-tau antibody molecule described herein may be used to treat subjects suffering from CTE. In some cases, methods of the present disclosure may be used to treat subjects suspected of developing CTE.

### Prion Diseases

Prion diseases, also known as transmissible spongiform encephalopathies (TSEs), are a group of rare progressive conditions affecting the nervous system. The related conditions are rare and are typically caused by mutations in the PRNP gene which enables production of the prion protein. Gene mutations lead to an abnormally structured prion protein. Alternatively, the abnormal prion may be acquired by exposure from an outside source, e.g. by consumption of beef products containing the abnormal prion protein. Abnormal prions are misfolded, causing the brain tissue to degenerate rapidly. Prion diseases include, but are not limited to, Creutzfeldt-Jakob disease (CJD), Gerstmann-Straussler-Scheinker syndrome (GSS), fatal insomnia (FFI), variably protease-sensitive prionopathy (VPSPr), and kuru. Prion diseases are rare. Approximately 350 cases of prion diseases are diagnosed in the US annually.

CJD is a degenerative brain disorder characterized by problems with muscular coordination, personality changes including mental impairment, impaired vision, involuntary muscle jerks, weakness and eventually coma. The most common categories of CJD are sporadic, hereditary due to a genetic mutation, and acquired. Sporadic CJD is the most common form affecting people with no known risk factors for the disease. The acquired form of CJD is transmitted by exposure of the brain and nervous system tissue to the prion. As an example, variant CJD (vCDJ) is linked to a bovine spongiform encephalopathy (BSE), also known as a ‘mad cow’ disease. CJD is fatal and patients typically die within one year of diagnosis.

Prion diseases are associated with an infectious agent consisting of an alternative conformational isoform of the prion protein, PrPSc. PrPSc replication is considered to occur through an induction of the infectious prion in the normal prion protein (PrPC). The replication occurs without a nucleic acid.

As of today, there is no therapy to manage or cure CJD, or other prion diseases. Typically, treatment is aimed at alleviating symptoms and increasing comfortability of the patient, e.g. with pain relievers. There remains a need for therapy affecting the underlying pathophysiology, such as antibody therapies targeting the prion protein.

In some embodiments, administration of AAV particles which encode an anti-tau antibody molecule described herein may be used to treat subjects suffering from a prion disease. In some cases, methods of the present disclosure may be used to treat subjects suspected of developing a prion disease.

### Neurodegeneration and Stroke

Neurodegenerative diseases and other diseases of the nervous system share many common features. Neurodegenerative diseases, in particular, are a group of conditions characterized by progressive loss of neuronal structure and function, ultimately leading to neuronal cell death. Neurons are the building blocks of the nervous system(s) and are generally not able to reproduce and/or be replaced, and therefore neuron damage and/or death is especially devastating. Other, non-degenerating diseases that lead to neuronal cell loss, such as stroke, have similarly debilitating outcomes. Targeting molecules that contribute to the deteriorating cell structure or function may prove beneficial generally for treatment of nervous system diseases, neurodegenerative disease and/or stroke.

Certain molecules are believed to have inhibitory effects on neurite outgrowth, contributing to the limited ability of the central nervous system to repair. Such molecules include, but are not limited to, myelin associated proteins, such as, but not limited to, RGM (Repulsive guidance molecule), NOGO (Neurite outgrowth inhibitor), NOGO receptor, MAG (myelin associated glycoprotein), and MAI (myelin associated inhibitor). In some embodiments, the vectored antibody delivery of the present disclosure is utilized to target the aforementioned antigens (e.g., neurite outgrowth inhibitors).

Many neurodegenerative diseases are associated with aggregation of misfolded proteins, including, but not limited to, alpha synuclein, tau, amyloid β, prion proteins, TDP-43, and huntingtin (see, e.g. De Genst et al., 2014, Biochim Biophys Acta; 1844(11):1907-1919, and Yu et al., 2013, Neurotherapeutics; 10(3): 459-472, references therein). The aggregation results from disease-specific conversion of soluble proteins to an insoluble, highly ordered fibrillary deposit. This conversion is thought to prevent the proper disposal or degradation of the misfolded protein, thereby leading to further aggregation. Conditions associated with alpha synuclein and tau may be referred to as “synucleinopathies” and “tauopathies”, respectively. In some embodiments, the vectored antibody delivery of the present disclosure is utilized to target the aforementioned antigens (e.g., misfolded or aggregated proteins).

In some embodiments, administration of AAV particles which encode an anti-tau antibody molecule described herein may be used to prevent, manage and/or treat tauopathies or tau associated disease.

### V. Dosing and Administration

### Administration

In some aspects, the present disclosure provides administration and/or delivery methods for vectors and viral particles, e.g., AAV particles, encoding an anti-tau antibody molecule for the prevention, treatment, or amelioration of diseases or disorders of the CNS. For example, administration of the AAV particles prevents, treats, or ameliorates tau-related disorders. Thus, robust widespread distribution of the anti-tau antibody molecule throughout the CNS and periphery is desired for maximal efficacy. Particular target tissues for administration or delivery include CNS tissues, brain tissue, and, more specifically, caudate-putamen, thalamus, superior colliculus, cortex, and corpus collosum. Particular embodiments provide administration and/or delivery of the AAV particles and AAV vector genomes described herein to caudate-putamen and/or substantia nigra. Other particular embodiments provide administration and/or delivery of the AAV particles and AAV vector genomes described herein to thalamus.

The AAV particles of the present disclosure may be administered by any route which results in a therapeutically effective outcome. These include, but are not limited to, enteral (into the intestine), gastroenteral, epidural (into the dura matter), oral (by way of the mouth), transdermal, peridural, intracerebral (into the cerebrum), intracerebroventricular (into the cerebral ventricles), intracranial (into the skull), picutaneous (application onto the skin), intradermal, (into the skin itself), subcutaneous (under the skin), nasal administration (through the nose), intravenous (into a vein), intravenous bolus, intravenous drip, intraarterial (into an artery), intramuscular (into a muscle), intracardiac (into the heart), intraosseous infusion (into the bone marrow), intraparenchymal (into the substance of), intrathecal (into the spinal canal), intraperitoneal, (infusion or injection into the peritoneum), intravesicular infusion, intravitreal, (through the eye), intracavernous injection (into a pathologic cavity) intracavitary (into the base of the penis), intravaginal administration, intrauterine, extra-amniotic administration, transdermal (diffusion through the intact skin for systemic distribution), transmucosal (diffusion through a mucous membrane), transvaginal, insufflation (snorting), sublingual, sublabial, enema, eye drops (onto the conjunctiva), in ear drops, auricular (in or by way of the ear), buccal (directed toward the cheek), conjunctival, cutaneous, dental (to a tooth or teeth), electro-osmosis, endocervical, endosinusial, endotracheal, extracorporeal, hemodialysis, infiltration, interstitial, intra-abdominal, intra-amniotic, intra-articular, intrabiliary, intrabronchial, intrabursal, intracartilaginous (within a cartilage), intracaudal (within the cauda equine), intracisternal (within the cisterna magna cerebellomedularis), intracorneal (within the cornea), dental intracoronal, intracoronary (within the coronary arteries), intracorporus cavernosum (within the dilatable spaces of the corporus cavernosa of the penis), intradiscal (within a disc), intraductal (within a duct of a gland), intraduodenal (within the duodenum), intradural (within or beneath the dura), intraepidermal (to the epidermis), intraesophageal (to the esophagus), intragastric (within the stomach), intragingival (within the gingivae), intraileal (within the distal portion of the small intestine), intralesional (within or introduced directly to a localized lesion), intraluminal (within a lumen of a tube), intralymphatic (within the lymph), intramedullary (within the marrow cavity of a bone), intrameningeal (within the meninges), intraocular (within the eye), intraovarian (within the ovary), intrapericardial (within the pericardium), intrapleural (within the pleura), intraprostatic (within the prostate gland), intrapulmonary (within the lungs or its bronchi), intrasinal (within the nasal or periorbital sinuses), intraspinal (within the vertebral column), intrasynovial (within the synovial cavity of a joint), intratendinous (within a tendon), intratesticular (within the testicle), intrathecal (within the cerebrospinal fluid at any level of the cerebrospinal axis), intrathoracic (within the thorax), intratubular (within the tubules of an organ), intratumor (within a tumor), intratympanic (within the aurus media), intravascular (within a vessel or vessels), intraventricular (within a ventricle), iontophoresis (by means of electric current where ions of soluble salts migrate into the tissues of the body), irrigation (to bathe or flush open wounds or body cavities), laryngeal (directly upon the larynx), nasogastric (through the nose and into the stomach), occlusive dressing technique (topical route administration which is then covered by a dressing which occludes the area), ophthalmic (to the external eye), oropharyngeal (directly to the mouth and pharynx), parenteral, percutaneous, periarticular, peridural, perineural, periodontal, rectal, respiratory (within the respiratory tract by inhaling orally or nasally for local or systemic effect), retrobulbar (behind the pons or behind the eyeball), soft tissue, subarachnoid, subconjunctival, submucosal, subpial, topical, transplacental (through or across the placenta), transtracheal (through the wall of the trachea), transtympanic (across or through the tympanic cavity), ureteral (to the ureter), urethral (to the urethra), vaginal, caudal block, diagnostic, nerve block, biliary perfusion, cardiac perfusion, photopheresis or spinal.

In some embodiments, AAV particles of the present disclosure are administered so as to be delivered to a target cell or tissue. Delivery to a target cell results in anti-tau antibody molecule expression. A target cell may be a CNS cell. Non-limiting examples of such cells and/or tissues include, dorsal root ganglia and dorsal columns, proprioceptive sensory neurons, Clark's column, gracile and cuneate nuclei, cerebellar dentate nucleus, corticospinal tracts and the cells comprising the same, Betz cells, and cells of the heart.

In some embodiments, compositions may be administered in a way that allows them to cross the blood-brain barrier, vascular barrier, or other epithelial barrier.

In some embodiments, delivery of an anti-tau antibody molecule by adeno-associated virus (AAV) particles to cells of the central nervous system (e.g., parenchyma) comprises infusion into cerebrospinal fluid (CSF). CSF is produced by specialized ependymal cells that comprise the choroid plexus located in the ventricles of the brain. CSF produced within the brain then circulates and surrounds the central nervous system including the brain and spinal cord. CSF continually circulates around the central nervous system, including the ventricles of the brain and subarachnoid space that surrounds both the brain and spinal cord, while maintaining a homeostatic balance of production and reabsorption into the vascular system. The entire volume of CSF is replaced approximately four to six times per day or approximately once every four hours, though values for individuals may vary.

In some embodiments, the AAV particles may be delivered by systemic delivery. In some embodiments, the systemic delivery may be by intravascular administration. In some embodiments, the systemic delivery may be by intravenous (IV) administration.

In some embodiments, the AAV particles may be delivered by intravenous delivery.

In some embodiments, the AAV particle is administered to the subject via focused ultrasound (FUS), e.g., coupled with the intravenous administration of microbubbles (FUS-MB), or MRI-guided FUS coupled with intravenous administration, e.g., as described in Terstappen et al. (Nat Rev Drug Discovery, https://doi.org/10.1038/s41573-021-00139-y (2021)), Burgess et al. (Expert Rev Neurother. 15(5): 477-491 (2015)), and/or Hsu et al. (PLOS One 8(2): 1-8), the contents of which are incorporated herein by reference in its entirety.

In some embodiments, the AAV particles may be delivered by injection into the CSF pathway. Non-limiting examples of delivery to the CSF pathway include intrathecal and intracerebroventricular administration.

In some embodiments, the AAV particles may be delivered by thalamic delivery.

In some embodiments, the AAV particles may be delivered by intracerebral delivery.

In some embodiments, the AAV particles may be delivered by intracardiac delivery.

In some embodiments, the AAV particles may be delivered by intracranial delivery.

In some embodiments, the AAV particles may be delivered by intra cisterna magna (ICM) delivery.

In some embodiments, the AAV particles may be delivered by direct (intraparenchymal) injection into an organ (e.g., CNS (brain or spinal cord)). In some embodiments, the intraparenchymal delivery may be to any region of the brain or CNS.

In some embodiments, the AAV particles may be delivered by intrastriatal injection.

In some embodiments, the AAV particles may be delivered into the putamen.

In some embodiments, the AAV particles may be delivered into the spinal cord.

In some embodiments, the AAV particles of the present disclosure may be administered to the ventricles of the brain.

In some embodiments, the AAV particles of the present disclosure may be administered to the ventricles of the brain by intracerebroventricular delivery.

In some embodiments, the AAV particles of the present disclosure may be administered by intramuscular delivery.

In some embodiments, the AAV particles of the present disclosure are administered by more than one route described above. As a non-limiting example, the AAV particles may be administered by intravenous delivery and thalamic delivery.

In some embodiments, the AAV particles of the present disclosure are administered by more than one route described above. As a non-limiting example, the AAV particles may be administered by intravenous delivery and intracerebral delivery.

In some embodiments, the AAV particles of the present disclosure are administered by more than one route described above. As a non-limiting example, the AAV particles may be administered by intravenous delivery and intracranial delivery.

In some embodiments, the AAV particles of the present disclosure are administered by more than one route described above. In some embodiments, the AAV particles of the present disclosure may be delivered by intrathecal and intracerebroventricular administration.

In some embodiments, the AAV particles may be delivered to a subject to improve and/or correct mitochondrial dysfunction.

In some embodiments, the AAV particles may be delivered to a subject to preserve neurons. The neurons may be primary and/or secondary sensory neurons. In some embodiments, AAV particles are delivered to dorsal root ganglia and/or neurons thereof.

In some embodiments, administration of the AAV particles may preserve and/or correct function in the sensory pathways.

In some embodiments, the AAV particles may be delivered via intravenous (IV), intracerebroventricular (ICV), intraparenchymal, and/or intrathecal (IT) infusion and the therapeutic agent may also be delivered to a subject via intramuscular (IM) limb infusion in order to deliver the therapeutic agent to the skeletal muscle. Delivery of AAVs by intravascular limb infusion is described by Gruntman and Flotte, Human Gene Therapy Clinical Development, 2015, 26(3), 159-164, the contents of which are herein incorporated by reference in their entirety.

In some embodiments, delivery of viral vector pharmaceutical compositions in accordance with the present disclosure to cells of the central nervous system (e.g., parenchyma) comprises a rate of delivery defined by VG/hour=mL/hour*VG/mL, wherein VG is viral genomes, VG/mL is composition concentration, and mL/hour is rate of infusion.

In some embodiments, delivery of AAV particle pharmaceutical compositions in accordance with the present disclosure to cells of the central nervous system (e.g., parenchyma) comprises infusion of up to 1 mL. In some embodiments, delivery of viral vector pharmaceutical compositions in accordance with the present disclosure to cells of the central nervous system (e.g., parenchyma) may comprise infusion of 0.0001, 0.0002, 0.001, 0.002, 0.003, 0.004, 0.005, 0.008, 0.010, 0.015, 0.020, 0.025, 0.030, 0.040, 0.050, 0.060, 0.070, 0.080, 0.090, 0.1, 0.2, 0.3, 0.4, 0.5, 0.6, 0.7, 0.8, or 0.9 mL.

In some embodiments, delivery of AAV particle pharmaceutical compositions in accordance with the present disclosure to cells of the central nervous system (e.g., parenchyma) comprises infusion of between about 1 mL to about 120 mL. In some embodiments, delivery of viral vector pharmaceutical compositions in accordance with the present disclosure to cells of the central nervous system (e.g., parenchyma) may comprise an infusion of 0.1, 1, 1.1, 1.2, 1.3, 1.4, 1.5, 1.6, 1.7, 1.8, 1.9, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, or 120 mL. In some embodiments delivery of AAV particles to cells of the central nervous system (e.g., parenchyma) comprises infusion of at least 3 mL. In some embodiments, delivery of AAV particles to cells of the central nervous system (e.g., parenchyma) consists of infusion of 3 mL. In some embodiments, delivery of AAV particles to cells of the central nervous system (e.g., parenchyma) comprises infusion of at least 10 mL. In some embodiments, delivery of AAV particles to cells of the central nervous system (e.g., parenchyma) consists of infusion of 10 mL.

In some embodiments, the volume of the AAV particle pharmaceutical composition delivered to the cells of the central nervous system (e.g., parenchyma) of a subject is 2 μl, 20 μl, 50 μl, 80 μl, 100 μl, 200 μl, 300 μl, 400 μl, 500 μl, 600 μl, 700 μl, 800 μl, 900 μl, 1000 μl, 1100 μl, 1200 μl, 1300 μl, 1400 μl, 1500 μl, 1600 μl, 1700 μl, 1800 μl, 1900 μl, 2000 μl, or more than 2000 μl.

In some embodiments, the volume of the AAV particle pharmaceutical composition delivered to a region in both hemispheres of a subject brain is 2 μl, 20 μl, 50 μl, 80 μl, 100 μl, 200 μl, 300 μl, 400 μl, 500 μl, 600 μl, 700 μl, 800 μl, 900 μl, 1000 μl, 1100 μl, 1200 μl, 1300 μl, 1400 μl, 1500 μl, 1600 μl, 1700 μl, 1800 μl, 1900 μl, 2000 μl, or more than 2000 μl. In some embodiments, the volume delivered to a region in both hemispheres is 200 μl. As another non-limiting example, the volume delivered to a region in both hemispheres is 900 μl. As yet another non-limiting example, the volume delivered to a region in both hemispheres is 1800 μl.

In certain embodiments, AAV particle or viral vector pharmaceutical compositions in accordance with the present disclosure may be administered at about 10 to about 600 μl/site, about 50 to about 500 μl/site, about 100 to about 400 μl/site, about 120 to about 300 μl/site, about 140 to about 200 μl/site, or about 160 μl/site.

In some embodiments, the total volume delivered to a subject may be split between one or more administration sites e.g., 1, 2, 3, 4, 5, or more than 5 sites. In some embodiments, the total volume is split between administration to the left and right hemisphere.

### Delivery of AAV Particles

In some embodiments, the AAV particles or pharmaceutical compositions of the present disclosure may be administered or delivered using the methods for treatment of disease described in U.S. Pat. No. 8,999,948, or International Publication No. WO2014178863, the contents of which are herein incorporated by reference in their entirety.

In some embodiments, the AAV particles or pharmaceutical compositions of the present disclosure may be administered or delivered using the methods for delivering gene therapy in Alzheimer's Disease or other neurodegenerative conditions as described in US Application No. 20150126590, the contents of which are herein incorporated by reference in their entirety.

In some embodiments, the AAV particles or pharmaceutical compositions of the present disclosure may be administered or delivered using the methods for delivery of a CNS gene therapy as described in U.S. Pat. Nos. 6,436,708, and 8,946,152, and International Publication No. WO2015168666, the contents of which are herein incorporated by reference in their entirety.

In some embodiments, the AAV particles of the present disclosure may be administered or delivered using the methods for the delivery of AAV virions described in European Patent Application No. EP1857552, the contents of which are herein incorporated by reference in their entirety.

In some embodiments, the AAV particle or pharmaceutical compositions of the present disclosure may be administered or delivered using the methods for delivering proteins using AAV vectors described in European Patent Application No. EP2678433, the contents of which are herein incorporated by reference in their entirety.

In some embodiments, the viral vector encoding an anti-tau antibody molecule may be administered or delivered using the methods for delivering DNA molecules using AAV vectors described in U.S. Pat. No. 5,858,351, the contents of which are herein incorporated by reference in their entirety.

In some embodiments, the AAV particle or pharmaceutical compositions of the present disclosure may be administered or delivered using the methods for delivering DNA to the bloodstream described in U.S. Pat. No. 6,211,163, the contents of which are herein incorporated by reference in their entirety.

In some embodiments, the viral vector encoding an anti-tau antibody molecule may be administered or delivered using the methods for delivering AAV virions described in U.S. Pat. No. 6,325,998, the contents of which are herein incorporated by reference in their entirety.

In some embodiments, the viral vector encoding an anti-tau antibody molecule may be administered or delivered using the methods for delivering DNA to muscle cells described in U.S. Pat. No. 6,335,011, the contents of which are herein incorporated by reference in their entirety.

In some embodiments, the viral vector encoding an anti-tau antibody molecule may be administered or delivered using the methods for delivering DNA to muscle cells and tissues described in U.S. Pat. No. 6,610,290, the contents of which are herein incorporated by reference in their entirety.

In some embodiments, the viral vector encoding an anti-tau antibody molecule may be administered or delivered using the methods for delivering DNA to muscle cells described in U.S. Pat. No. 7,704,492, the contents of which are herein incorporated by reference in their entirety.

In some embodiments, the viral vector encoding an anti-tau antibody molecule may be administered or delivered using the methods for delivering a payload to skeletal muscles described in U.S. Pat. No. 7,112,321, the contents of which are herein incorporated by reference in their entirety.

In some embodiments, the AAV particle or pharmaceutical compositions of the present disclosure may be administered or delivered using the methods for delivering a payload to the central nervous system described in U.S. Pat. No. 7,588,757, the contents of which are herein incorporated by reference in their entirety.

In some embodiments, the AAV particle or pharmaceutical compositions of the present disclosure may be administered or delivered using the methods for delivering a payload described in U.S. Pat. No. 8,283,151, the contents of which are herein incorporated by reference in their entirety.

In some embodiments, the AAV particle or pharmaceutical compositions of the present disclosure may be administered or delivered using the methods for delivering a payload for the treatment of Alzheimer disease described in U.S. Pat. No. 8,318,687, the contents of which are herein incorporated by reference in their entirety.

In some embodiments, the AAV particle or pharmaceutical compositions of the present disclosure may be administered or delivered using the methods for delivering a payload described in International Patent Publication No. WO2012144446, the contents of which are herein incorporated by reference in their entirety.

In some embodiments, the AAV particle or pharmaceutical compositions of the present disclosure may be administered or delivered using the methods for delivering a payload using a glutamic acid decarboxylase (GAD) delivery vector described in International Patent Publication No. WO2001089583, the contents of which are herein incorporated by reference in their entirety.

In some embodiments, the AAV particle or pharmaceutical compositions of the present disclosure may be administered or delivered using the methods for delivering a payload to neural cells described in International Patent Publication No. WO2012057363, the contents of which are herein incorporated by reference in their entirety.

In some embodiments, the AAV particle or pharmaceutical compositions of the present disclosure may be administered or delivered using the methods for delivering a payload described in International Patent Publication No. WO2001096587, the contents of which are herein incorporated by reference in their entirety.

In some embodiments, the AAV particle or pharmaceutical compositions of the present disclosure may be administered or delivered using the methods for delivering a payload to muscle tissue described in International Patent Publication No. WO2002014487, the contents of which are herein incorporated by reference in their entirety.

In some embodiments, a catheter may be used to administer the AAV particles. In certain embodiments, the catheter or cannula may be located at more than one site in the spine for multi-site delivery. The viral particles encoding may be delivered in a continuous and/or bolus infusion. Each site of delivery may be a different dosing regimen or the same dosing regimen may be used for each site of delivery. In some embodiments, the sites of delivery may be in the cervical and the lumbar region. In some embodiments, the sites of delivery may be in the cervical region. In some embodiments, the sites of delivery may be in the lumbar region.

In some embodiments, a subject may be analyzed for spinal anatomy and pathology prior to delivery of the AAV particles described herein. As a non-limiting example, a subject with scoliosis may have a different dosing regimen and/or catheter location compared to a subject without scoliosis.

In some embodiments, the delivery method and duration is chosen to provide broad transduction in the spinal cord. In some embodiments, intrathecal delivery is used to provide broad transduction along the rostral-caudal length of the spinal cord. In some embodiments, multi-site infusions provide a more uniform transduction along the rostral-caudal length of the spinal cord.

### Delivery to Cells

In some aspects, the present disclosure provides a method of delivering to a cell or tissue any of the above-described AAV particles, comprising contacting the cell or tissue with said AAV particle or contacting the cell or tissue with a formulation comprising said AAV particle, or contacting the cell or tissue with any of the described compositions, including pharmaceutical compositions. The method of delivering the AAV particle to a cell or tissue can be accomplished in vitro, ex vivo, or in vivo.

### Delivery to Subjects

In some aspects, the present disclosure additionally provides a method of delivering to a subject, including a mammalian subject, any of the above-described AAV particles comprising administering to the subject said AAV particle, or administering to the subject a formulation comprising said AAV particle, or administering to the subject any of the described compositions, including pharmaceutical compositions.

In some embodiments, the AAV particles may be delivered to bypass anatomical blockages such as, but not limited to the blood brain barrier.

In some embodiments, the AAV particles may be formulated and delivered to a subject by a route which increases the speed of drug effect as compared to oral delivery.

In some embodiments, the AAV particles may be delivered by a method to provide uniform transduction of the spinal cord and dorsal root ganglion (DRG). In some embodiments, the AAV particles may be delivered using intrathecal infusion.

In some embodiments, a subject may be administered the AAV particles described herein using a bolus infusion. As used herein, a “bolus infusion” means a single and rapid infusion of a substance or composition.

In some embodiments, the AAV particles encoding an anti-tau antibody molecule may be delivered in a continuous and/or bolus infusion. Each site of delivery may be a different dosing regimen or the same dosing regimen may be used for each site of delivery. As a non-limiting example, the sites of delivery may be in the cervical and the lumbar region. As another non-limiting example, the sites of delivery may be in the cervical region. As another non-limiting example, the sites of delivery may be in the lumbar region.

In some embodiments, the AAV particles may be delivered to a subject via a single route administration.

In some embodiments, the AAV particles may be delivered to a subject via a multi-site route of administration. For example, a subject may be administered the AAV particles at 2, 3, 4, 5, or more than 5 sites.

In some embodiments, a subject may be administered the AAV particles described herein using sustained delivery over a period of minutes, hours or days. The infusion rate may be changed depending on the subject, distribution, formulation or another delivery parameter known to those in the art.

In some embodiments, if continuous delivery (continuous infusion) of the AAV particles is used, the continuous infusion may be for 1 hour, 2, hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, or more than 24 hours.

In some embodiments, the intracranial pressure may be evaluated prior to administration. The route, volume, AAV particle concentration, infusion duration and/or vector titer may be optimized based on the intracranial pressure of a subject.

In some embodiments, the AAV particles may be delivered by systemic delivery. In some embodiments, the systemic delivery may be by intravascular administration.

In some embodiments, the AAV particles may be delivered by injection into the CSF pathway. Non-limiting examples of delivery to the CSF pathway include intrathecal and intracerebroventricular administration.

In some embodiments, the AAV particles may be delivered by direct (intraparenchymal) injection into the substance of an organ, e.g., one or more regions of the brain.

In some embodiments, the AAV particles may be delivered by subpial injection into the spinal cord. For example, subjects may be placed into a spinal immobilization apparatus. A dorsal laminectomy may be performed to expose the spinal cord. Guiding tubes and XYZ manipulators may be used to assist catheter placement. Subpial catheters may be placed into the subpial space by advancing the catheter from the guiding tube and AAV particles may be injected through the catheter (Miyanohara et al., Mol Ther Methods Clin Dev. 2016; 3: 16046). In some cases, the AAV particles may be injected into the cervical subpial space. In some cases, the AAV particles may be injected into the thoracic subpial space.

In some embodiments, the AAV particles may be delivered by direct injection to the CNS of a subject. In some embodiments, direct injection is intracerebral injection, intraparenchymal injection, intrathecal injection, intra-cisterna magna injection, or any combination thereof. In some embodiments, direct injection to the CNS of a subject comprises convection enhanced delivery (CED). In some embodiments, administration comprises peripheral injection. In some embodiments, peripheral injection is intravenous injection.

In some embodiments, the AAV particles may be delivered to a subject in order to increase anti-tau antibody molecule levels in the caudate-putamen, thalamus, superior colliculus, cortex, and/or corpus callosum.

In some embodiments, the AAV particles may be delivered to a subject in order to increase the anti-tau antibody molecule levels in the caudate, putamen, thalamus, superior colliculus, cortex, and/or corpus callosum by transducing cells in these CNS regions.

In some embodiments, delivery of AAV particles comprising a viral genome encoding an anti-tau antibody molecule described herein to neurons in the caudate-putamen, thalamus, superior colliculus, cortex, and/or corpus callosum will lead to an increased expression of an anti-tau antibody molecule. The increased expression may lead to improved survival and function of various cell types in these CNS regions and subsequent improvement of tau-related disorder symptoms.

In particular embodiments, the AAV particles may be delivered to a subject in order to establish widespread distribution of an anti-tau antibody molecule throughout the nervous system by administering the AAV particles to the thalamus of the subject.

### Dosing

In some aspects, the present disclosure provides methods comprising administering viral vectors and their payloads in accordance with the disclosure to a subject in need thereof. Viral vector pharmaceutical, imaging, diagnostic, or prophylactic compositions thereof, may be administered to a subject using any amount and any route of administration effective for preventing, treating, diagnosing, or imaging a disease, disorder, and/or condition. In some embodiments, the disease, disorder, and/or condition is a tau-related disorder. The exact amount required will vary from subject to subject, depending on the species, age, and general condition of the subject, the severity of the disease, the particular composition, its mode of administration, its mode of activity, and the like. Compositions in accordance with the disclosure are typically formulated in unit dosage form for ease of administration and uniformity of dosage. It will be understood, however, that the total daily usage of the compositions of the present disclosure may be decided by the attending physician within the scope of sound medical judgment. The specific therapeutically effective, prophylactically effective, or appropriate imaging dose level for any particular patient will depend upon a variety of factors including the disorder being treated and the severity of the disorder; the activity of the specific compound employed; the specific composition employed; the age, body weight, general health, sex, and diet of the patient; the time of administration, route of administration, and rate of excretion of the specific peptide(s) employed; the duration of the treatment; drugs used in combination or coincidental with the specific compound employed; and like factors well known in the medical arts.

In certain embodiments, AAV particle pharmaceutical compositions in accordance with the present disclosure may be administered at dosage levels sufficient to deliver an anti-tau antibody molecule from about 0.0001 mg/kg to about 100 mg/kg, from about 0.001 mg/kg to about 0.05 mg/kg, from about 0.005 mg/kg to about 0.05 mg/kg, from about 0.001 mg/kg to about 0.005 mg/kg, from about 0.05 mg/kg to about 0.5 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day, to obtain the desired therapeutic, diagnostic, prophylactic, or imaging effect. It will be understood that the above dosing concentrations may be converted to VG or viral genomes per kg or into total viral genomes administered by one of skill in the art.

In certain embodiments, the desired dosage may be delivered using multiple administrations (e.g., two, three, four, five, six, seven, eight, nine, ten, eleven, twelve, thirteen, fourteen, or more administrations). When multiple administrations are employed, split dosing regimens such as those described herein may be used. As used herein, a “split dose” is the division of single unit dose or total daily dose into two or more doses, e.g., two or more administrations of the single unit dose. As used herein, a “single unit dose” is a dose of any therapeutic composition administered in one dose/at one time/single route/single point of contact, i.e., single administration event. In some embodiments, a single unit dose is provided as a discrete dosage form (e.g., a tablet, capsule, patch, loaded syringe, vial, etc.). As used herein, a “total daily dose” is an amount given or prescribed in 24-hour period. It may be administered as a single unit dose. The viral particles may be formulated in buffer only or in a formulation described herein.

A pharmaceutical composition described herein can be formulated into a dosage form described herein, such as a topical, intranasal, pulmonary, intratracheal, or injectable (e.g., intravenous, intraocular, intravitreal, intramuscular, intracardiac, intraperitoneal, and/or subcutaneous).

In some embodiments, delivery of the AAV particles described herein results in minimal serious adverse events (SAEs) as a result of the delivery of the AAV particles.

In some embodiments, delivery of AAV particle pharmaceutical compositions in accordance with the present disclosure to cells of the central nervous system (e.g., parenchyma) may comprise a total concentration between about 1×10⁶ VG/mL and about 1×10¹⁶ VG/mL. In some embodiments, delivery may comprise a composition concentration of about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 1.6×10¹¹, 1.8×10¹¹, 2×10¹¹, 3×10¹¹, 4×10¹¹, 5×10¹¹, 5.5×10¹¹, 6×10¹¹, 7×10¹¹, 8×10¹¹, 9×10¹¹, 0.8×10¹², 0.83×10¹², 1×10¹², 1.1×10¹², 1.2×10¹², 1.3×10¹², 1.4×10¹², 1.5×10¹², 1.6×10¹², 1.7×10¹², 1.8×10¹², 1.9×10¹², 2×10¹², 2.1×10², 2.2×10¹², 2.3×10¹², 2.4×10¹², 2.5×10¹², 2.6×10¹², 2.7×10¹², 2.8×10¹², 2.9×10¹², 3×10¹², 3.1×10², 3.2×10¹², 3.3×10¹², 3.4×10¹², 3.5×10¹², 3.6×10¹², 3.7×10¹², 3.8×10¹², 3.9×10¹², 4×10¹², 4.1×10², 4.2×10¹², 4.3×10¹², 4.4×10¹², 4.5×10¹², 4.6×10¹², 4.7×10¹², 4.8×10¹², 4.9×10¹², 5×10², 6×10¹², 7×10¹², 8×10¹², 9×10¹², 1×10¹³, 2×10¹³, 2.3×10¹³, 3×10¹³, 4×10¹³, 5×10¹³, 6×10¹³, 7×10¹³, 8×10¹³, 9×10¹³, 1×10¹⁴, 1.9×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, or 1×10¹⁶ VG/mL.

In some embodiments, delivery of AAV particle pharmaceutical compositions in accordance with the present disclosure to cells of the central nervous system (e.g., parenchyma) may comprise a total concentration per subject between about 1×10⁶ VG and about 1×10¹⁶ VG. In some embodiments, delivery may comprise a composition concentration of about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 1.6×10¹¹, 2×10¹¹, 2.1×10¹¹, 2.2×10¹¹, 2.3×10¹¹, 2.4×10¹¹, 2.5×10¹¹, 2.6×10¹¹, 2.7×10¹¹, 2.8×10¹¹, 2.9×10¹¹, 3×10¹¹, 4×10¹¹, 4.6×10¹¹, 5×10¹¹, 6×10¹¹, 7×10¹¹, 7.1×10¹¹, 7.2×10¹¹, 7.3×10¹¹, 7.4×10¹¹, 7.5×10¹¹, 7.6×10¹¹, 7.7×10¹¹, 7.8×10¹¹, 7.9×10¹¹, 8×10¹¹, 9×10¹¹, 1×10¹², 1.1×10¹², 1.2×10¹², 1.3×10¹², 1.4×10¹², 1.5×10¹², 1.6×10¹², 1.7×10¹², 1.8×10¹², 1.9×10¹², 2×10¹², 2.3×10¹², 3×10¹², 4×10¹², 4.1×10², 4.2×10¹², 4.3×10¹², 4.4×10¹², 4.5×10¹², 4.6×10¹², 4.7×10¹², 4.8×10¹², 4.9×10¹², 5×10¹², 6×10¹², 7×10¹², 8×10¹², 8.1×10², 8.2×10¹², 8.3×10¹², 8.4×10¹², 8.5×10¹², 8.6×10¹², 8.7×10¹², 8.8×10¹², 8.9×10¹², 9×10¹², 1×10¹³, 2×10¹³, 3×10¹³, 4×10¹³, 5×10¹³, 6×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, or 1×10¹⁶ VG/subject.

In some embodiments, delivery of AAV particles to cells of the central nervous system (e.g., parenchyma) may comprise a total dose between about 1×10⁶ VG and about 1×10¹⁶ VG. In some embodiments, delivery may comprise a total dose of about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 1.9×10¹⁰, 2×10¹⁰, 3×10¹⁰, 3.73×10¹⁰, 4×10¹⁰, 5×10¹⁰, 6×10¹⁰, 7×10¹⁰, 8×10¹⁰, 9×10¹⁰, 1×10¹¹, 2×10¹¹, 2.5×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, 2×10¹³, 3×10¹³, 4×10¹³, 5×10¹³, 6×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, or 1×10¹⁶ VG.

### Combinations

The AAV particles may be used in combination with one or more other therapeutic, prophylactic, diagnostic, or imaging agents. The phrase “in combination with,” is not intended to require that the agents must be administered at the same time and/or formulated for delivery together, although these methods of delivery are within the scope of the present disclosure. Compositions can be administered concurrently with, prior to, or subsequent to, one or more other desired therapeutics or medical procedures. In general, each agent will be administered at a dose and/or on a time schedule determined for that agent. In some embodiments, the present disclosure encompasses the delivery of pharmaceutical, prophylactic, diagnostic, or imaging compositions in combination with agents that may improve their bioavailability, reduce and/or modify their metabolism, and/or modify their distribution within the body.

The therapeutic agents may be approved by the US Food and Drug Administration or may be in clinical trial or at the preclinical research stage. The therapeutic agents may utilize any therapeutic modality known in the art, with non-limiting examples including gene silencing or interference (i.e., miRNA, siRNA, RNAi, shRNA), gene editing (i.e., TALEN, CRISPR/Cas9 systems, zinc finger nucleases), and gene, protein or enzyme replacement.

### Measurement of Expression

Expression of an anti-tau antibody molecule from viral genomes may be determined using various methods known in the art such as, but not limited to immunochemistry (e.g., IHC), enzyme-linked immunosorbent assay (ELISA), affinity ELISA, ELISPOT, flow cytometry, immunocytology, surface plasmon resonance analysis, kinetic exclusion assay, liquid chromatography-mass spectrometry (LCMS), high-performance liquid chromatography (HPLC), BCA assay, immunoelectrophoresis, Western blot, SDS-PAGE, protein immunoprecipitation, PCR, and/or in situ hybridization (ISH). In some embodiments, transgenes encoding an anti-tau antibody molecule delivered in different AAV capsids may have different expression levels in different CNS tissues.

### VI. Kits and Devices

### Kits

In some aspects, the present disclosure provides a variety of kits for conveniently and/or effectively carrying out methods of the present disclosure. Typically, kits will comprise sufficient amounts and/or numbers of components to allow a user to perform multiple treatments of a subject(s) and/or to perform multiple experiments.

Any of the vectors, constructs, or anti-tau antibody molecules of the present disclosure may be comprised in a kit. In some embodiments, kits may further include reagents and/or instructions for creating and/or synthesizing compounds and/or compositions of the present disclosure. In some embodiments, kits may also include one or more buffers. In some embodiments, kits of the disclosure may include components for making protein or nucleic acid arrays or libraries and thus, may include, for example, solid supports.

In some embodiments, kit components may be packaged either in aqueous media or in lyophilized form. The container means of the kits will generally include at least one vial, test tube, flask, bottle, syringe or other container means, into which a component may be placed, and suitably aliquoted. Where there is more than one kit component, (labeling reagent and label may be packaged together), kits may also generally contain second, third or other additional containers into which additional components may be separately placed. In some embodiments, kits may also comprise second container means for containing sterile, pharmaceutically acceptable buffers and/or other diluents. In some embodiments, various combinations of components may be comprised in one or more vial. Kits of the present disclosure may also typically include means for containing compounds and/or compositions of the present disclosure, e.g., proteins, nucleic acids, and any other reagent containers in close confinement for commercial sale. Such containers may include injection or blow-molded plastic containers into which desired vials are retained.

In some embodiments, kit components are provided in one and/or more liquid solutions. In some embodiments, liquid solutions are aqueous solutions, with sterile aqueous solutions being particularly used. In some embodiments, kit components may be provided as dried powder(s). When reagents and/or components are provided as dry powders, such powders may be reconstituted by the addition of suitable volumes of solvent. In some embodiments, it is envisioned that solvents may also be provided in another container means. In some embodiments, labeling dyes are provided as dried powders. In some embodiments, it is contemplated that 10, 20, 30, 40, 50, 60, 70, 80, 90, 100, 120, 120, 130, 140, 150, 160, 170, 180, 190, 200, 300, 400, 500, 600, 700, 800, 900, 1000 micrograms or at least or at most those amounts of dried dye are provided in kits of the disclosure. In such embodiments, dye may then be resuspended in any suitable solvent, such as DMSO.

In some embodiments, kits may include instructions for employing kit components as well the use of any other reagent not included in the kit. Instructions may include variations that may be implemented.

### Devices

In some embodiments, compounds and/or compositions of the present disclosure may be combined with, coated onto or embedded in a device. Devices may include, but are not limited to, dental implants, stents, bone replacements, artificial joints, valves, pacemakers and/or other implantable therapeutic device.

The present disclosure provides for devices which may incorporate viral vectors that encode one or more anti-tau antibody molecules. These devices contain in a stable formulation the viral vectors which may be immediately delivered to a subject in need thereof, such as a human patient.

Devices for administration may be employed to deliver the viral vectors encoding anti-tau antibody molecules of the present disclosure according to single, multi- or split-dosing regimens taught herein.

Method and devices known in the art for multi-administration to cells, organs and tissues are contemplated for use in conjunction with the methods and compositions disclosed herein as embodiments of the present disclosure.

### VII. Definitions

At various places in the present specification, substituents of compounds of the present disclosure are disclosed in groups or in ranges. It is specifically intended that the present disclosure include each and every individual sub-combination of the members of such groups and ranges. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. As another example, a range such as 95-99% identity, includes something with 95%, 96%, 97%, 98%, or 99% identity, and includes subranges such as 96-99%, 96-98%, 96-97%, 97-99%, 97-98%, and 98-99% identity. This applies regardless of the breadth of the range.

The following is a non-limiting list of term definitions.

A and an: As used herein, the terms “a” and “an” refers to one or to more than one (i.e., to at least one) of the grammatical object of the article. By way of example, “an element” means one element or more than one element.

About and approximately: As used herein, the terms “about” and “approximately” when referring to a measurable value such as an amount, a temporal duration, and the like, are meant to encompass variations of ±20% or in some instances ±10%, or in some instances ±5%, or in some instances ±1%, or in some instances ±0.1% from the specified value, as such variations are appropriate to perform the disclosed methods.

Adeno-associated virus: As used herein, the term “adeno-associated virus” or “AAV” refers to members of the dependovirus genus or a variant, e.g., a functional variant, thereof. In some embodiments, the AAV is wildtype, or naturally occurring. In some embodiments, the AAV is recombinant.

AAVParticle: As used herein, an “AAV particle” refers to a particle or a virion comprising an AAV capsid, e.g., an AAV capsid variant, and a polynucleotide, e.g., a viral genome or a vector genome. In some embodiments, the viral genome of the AAV particle comprises at least one payload region and at least one ITR. In some embodiments, an AAV particle of the disclosure is an AAV particle comprising an AAV capsid polypeptide, e.g., a parent capsid sequence with at least one peptide insert. In some embodiments, the AAV particle is capable of delivering a nucleic acid, e.g., a payload region, encoding a payload to cells, typically, mammalian, e.g., human, cells. In some embodiments, an AAV particle of the present disclosure may be produced recombinantly. In some embodiments, an AAV particle may be derived from any serotype, described herein or known in the art, including combinations of serotypes (e.g., “pseudotyped” AAV) or from various genomes (e.g., single stranded or self-complementary). In some embodiments, the AAV particle may be replication defective and/or targeted. In some embodiments, the AAV particle may comprises a peptide present, e.g., inserted into, the capsid to enhance tropism for a desired target tissue. It is to be understood that reference to the AAV particle of the disclosure also includes pharmaceutical compositions thereof, even if not explicitly recited.

Administered in combination: As used herein, the term “administered in combination” or “delivered in combination” or “combined administration” refers to exposure of two or more agents (e.g., AAV) administered at the same time or within an interval such that the subject is at some point in time exposed to both agents and/or such that there is an overlap in the effect of each agent on the patient. In some embodiments, at least one dose of one or more agents is administered within about 24 hours, 12 hours, 6 hours, 3 hours, 1 hour, 30 minutes, 15 minutes, 10 minutes, 5 minutes, or 1 minute of at least one dose of one or more other agents. In some embodiments, administration occurs in overlapping dosage regimens. As used herein, the term “dosage regimen” refers to a plurality of doses spaced apart in time. Such doses may occur at regular intervals or may include one or more hiatuses in administration. In some embodiments, the administration of individual doses of one or more compounds and/or compositions of the present disclosure, as described herein, are spaced sufficiently closely together such that a combinatorial (e.g., a synergistic) effect is achieved.

Amelioration: As used herein, the term “amelioration” or “ameliorating” refers to a lessening of severity of at least one indicator of a condition or disease. For example, in the context of a neurodegenerative disorder, amelioration includes the reduction or stabilization of neuron loss.

Antibody or antibody molecule: As used herein, the term “antibody” or “antibody molecule” refers to a protein comprising at least one immunoglobulin variable domain sequence. Antibodies, for example, can be polyclonal or monoclonal, multiple or single chain, or intact immunoglobulins, and may be derived from natural sources or from recombinant sources. Antibodies can be tetramers of immunoglobulin molecules. In some embodiments, an antibody molecule comprises a full length antibody, or a full length immunoglobulin chain. In some embodiments, an antibody molecule comprises an antigen binding or functional fragment of a full length antibody, or a full length immunoglobulin chain. In some embodiments, the term antibody includes functional fragments thereof. In some embodiments, constant regions of the antibodies can be altered, e.g., mutated, to modify the properties of the antibody (e.g., to increase or decrease one or more of: Fc receptor binding, antibody glycosylation, the number of cysteine residues, effector cell function, or complement function).

Antibody fragment: As used herein, the term “antibody fragment” or “fragment” (when used in the context of an antibody molecule, e.g., “antibody molecule or a fragment thereof”) refers to at least one portion of an intact antibody, or recombinant variants thereof, and refers to the antigen binding domain, for example, an antigenic determining variable region of an intact antibody, that is sufficient to confer recognition and specific binding of the antibody fragment to a target, such as an antigen. Examples of antibody fragments include, but are not limited to, Fab, Fab′, F(ab′)2, and Fv fragments, scFv antibody fragments, linear antibodies, single domain antibodies such as sdAb (either VL or VH), camelid VHH domains, and multi-specific molecules formed from antibody fragments such as a bivalent fragment comprising two or more, for example, two, Fab fragments linked by a disulfide bridge at the hinge region, or two or more, for example, two isolated CDR or other epitope binding fragments of an antibody linked. An antibody fragment can also be incorporated into single domain antibodies, maxibodies, minibodies, nanobodies, intrabodies, diabodies, triabodies, tetrabodies, v-NAR and bis-scFv (see, for example, Hollinger and Hudson, Nature Biotechnology 23:1126-1136, 2005). Antibody fragments can also be grafted into scaffolds based on polypeptides such as a fibronectin type III (Fn3) (see U.S. Pat. No. 6,703,199, which describes fibronectin polypeptide minibodies).

Antibody heavy chain or heavy chain: As used herein, the term “antibody heavy chain,” or “heavy chain” refers to the larger of the two types of polypeptide chains present in antibody molecules in their naturally occurring conformations, and which normally determines the class to which the antibody belongs.

Antibody light chain or light chain: As used herein, the term “antibody light chain,” or “light chain” refers to the smaller of the two types of polypeptide chains present in antibody molecules in their naturally occurring conformations. Kappa (κ) and lambda (λ) light chains refer to the two major antibody light chain isotypes.

Antigen binding site: As used herein, the term “antigen-binding site” refers to the part of an antibody molecule that comprises determinants that form an interface that binds to a polypeptide, or an epitope thereof. With respect to proteins (or protein mimetics), the antigen-binding site typically includes one or more loops (of at least, e.g., four amino acids or amino acid mimics) that form an interface that binds to a polypeptide. Typically, the antigen-binding site of an antibody molecule includes at least one or two CDRs and/or hypervariable loops, or more typically at least three, four, five or six CDRs and/or hypervariable loops.

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.25%, 0.1%, or less in either direction (greater than or less than) of the stated reference value unless otherwise stated or otherwise evident from the context (except where such number would exceed 100% of a possible value).

Bispecific antibody: As used herein, the term “bispecific antibody” refers to an antibody that has specificity for no more than two antigens. A bispecific antibody molecule is characterized by a first immunoglobulin variable domain sequence which has binding specificity for a first epitope and a second immunoglobulin variable domain sequence that has binding specificity for a second epitope. In some embodiments, the first and second epitopes are on the same antigen, e.g., the same protein (or subunit of a multimeric protein). In some embodiments, the first and second epitopes overlap or substantially overlap. In some embodiments, the first and second epitopes do not overlap or do not substantially overlap (e.g., a biparatopic antibody). In some embodiments, the first and second epitopes are on different antigens, e.g., different proteins (or different subunits of a multimeric protein). In some embodiments, a bispecific antibody is able to bind two different antigens simultaneously or sequentially. Methods for making bispecific antibodies are well known in the art. Various formats for combining two antibodies are also known in the art. Forms of bispecific antibodies of the invention include, but are not limited to, a diabody, a single-chain diabody, Fab dimerization (Fab-Fab), Fab-scFv, and a tandem antibody, as known to those of skill in the art.

Capsid: As used herein, the term “capsid” refers to the exterior, e.g., a protein shell, of a virus particle, e.g., an AAV particle, that is substantially (e.g., >50%, >60%, >70%, >80%, >90%, >95%, >99%, or 100%) protein. In some embodiments, the capsid is an AAV capsid comprising an AAV capsid protein described herein, e.g., a VP1, VP2, and/or VP3 polypeptide. The AAV capsid protein can be a wild-type AAV capsid protein or a variant, e.g., a structural and/or functional variant from a wild-type or a reference capsid protein, referred to herein as an “AAV capsid variant.” In some embodiments, the AAV capsid variant described herein has the ability to enclose, e.g., encapsulate, a viral genome and/or is capable of entry into a cell, e.g., a mammalian cell. In some embodiments, the AAV capsid variant described herein may have modified tropism compared to that of a wild-type AAV capsid, e.g., the corresponding wild-type capsid.

Complementarity determining region or CDR: As used herein, the terms “complementarity determining region” or “CDR,” as used herein, refer to the sequences of amino acids within antibody variable regions which confer antigen specificity and binding affinity. For example, in general, there are three CDRs in each heavy chain variable region (for example, HCDR1, HCDR2, and HCDR3) and three CDRs in each light chain variable region (LCDR1, LCDR2, and LCDR3). The precise amino acid sequence boundaries of a given CDR can be determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme), Al-Lazikani et al., (1997) JMB 273,927-948 (“Chothia” numbering scheme), or a combination thereof. In a combined Kabat and Chothia numbering scheme, in some embodiments, the CDRs correspond to the amino acid residues that are part of a Kabat CDR, a Chothia CDR, or both.

Encapsulate: As used herein, the term “encapsulate” means to enclose, surround or encase. As an example, a capsid protein, e.g., an AAV capsid variant, often encapsulates a viral genome. In some embodiments, encapsulate within a capsid, e.g., an AAV capsid variant, encompasses 100% coverage by a capsid, as well as less than 100% coverage, e.g., 95%, 90%, 85%, 80%, 70%, 60% or less. For example, gaps or discontinuities may be present in the capsid so long as the viral genome is retained in the capsid, e.g., prior to entry into a cell.

Central Nervous System or CNS: As used herein, “central nervous system” or “CNS” refers to one of the two major subdivisions of the nervous system, which in vertebrates includes the brain and spinal cord. The central nervous system coordinates the activity of the entire nervous system.

Cervical Region: As used herein, “cervical region” refers to the region of the spinal cord comprising the cervical vertebrae C1, C2, C3, C4, C5, C6, C7, and C8.

Chimeric Receptor Antigen: The term “Chimeric Antigen Receptor” or alternatively a “CAR” refers to a recombinant polypeptide construct comprising at least an extracellular antigen binding domain, a transmembrane domain and a cytoplasmic signaling domain (also referred to herein as “an intracellular signaling domain”) comprising a functional signaling domain derived from a stimulatory molecule. In some embodiments, the domains in the CAR polypeptide construct are in the same polypeptide chain, for example, comprise a chimeric fusion protein. In some embodiments, the domains in the CAR polypeptide construct are not contiguous with each other, for example, are in different polypeptide chains.

CNS tissue: As used herein, “CNS tissue” or “CNS tissues” refers to the tissues of the central nervous system, which in vertebrates, include the brain and spinal cord and sub-structures thereof.

CNS structures: As used herein, “CNS structures” refers to structures of the central nervous system and sub-structures thereof. Non-limiting examples of structures in the spinal cord may include, ventral horn, dorsal horn, white matter, and nervous system pathways or nuclei within. Non-limiting examples of structures in the brain include, forebrain, midbrain, hindbrain, diencephalon, telencephalon, myelencephalon, metencephalon, mesencephalon, prosencephalon, rhombencephalon, cortices, frontal lobe, parietal lobe, temporal lobe, occipital lobe, cerebrum, thalamus, hypothalamus, tectum, tegmentum, cerebellum, pons, medulla, amygdala, hippocampus, basal ganglia, corpus callosum, pituitary gland, putamen, striatum, ventricles and sub-structures thereof.

CNS Cells: As used herein, “CNS cells” refers to cells of the central nervous system and sub-structures thereof. Non-limiting examples of CNS cells include, neurons and sub-types thereof, glia, microglia, oligodendrocytes, ependymal cells and astrocytes. Non-limiting examples of neurons include sensory neurons, motor neurons, interneurons, unipolar cells, bipolar cells, multipolar cells, pseudounipolar cells, pyramidal cells, basket cells, stellate cells, Purkinje cells, Betz cells, amacrine cells, granule cell, ovoid cell, medium aspiny neurons and large aspiny neurons.

Codon optimization: As used herein, the term “codon optimization” refers to a process of changing codons of a given gene in such a manner that the polypeptide sequence encoded by the gene remains the same while the changed codons improve the process of expression of the polypeptide sequence. For example, if the polypeptide is of a human protein sequence and expressed in E. coli, expression will often be improved if codon optimization is performed on the DNA sequence to change the human codons to codons that are more effective for expression in E. coli.

Conservative amino acid substitution: As used herein, a “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

Conserved: As used herein, the term “conserved” refers to nucleotides or amino acid residues of polynucleotide or polypeptide sequences, respectively, that are those that occur unaltered in the same position of two or more sequences being compared. Nucleotides or amino acids that are relatively conserved are those that are conserved among more related sequences than nucleotides or amino acids appearing elsewhere in the sequences.

In some embodiments, two or more sequences are said to be “completely conserved” if they are 100% identical to one another. In some embodiments, two or more sequences are said to be “highly conserved” if they are at least 70% identical, at least 80% identical, at least 90% identical, or at least 95% identical to one another. In some embodiments, two or more sequences are said to be “highly conserved” if they are about 70% identical, about 80% identical, about 90% identical, about 95%, about 98%, or about 99% identical to one another. In some embodiments, two or more sequences are said to be “conserved” if they are at least 30% identical, at least 40% identical, at least 50% identical, at least 60% identical, at least 70% identical, at least 80% identical, at least 90% identical, or at least 95% identical to one another. In some embodiments, two or more sequences are said to be “conserved” if they are about 30% identical, about 40% identical, about 50% identical, about 60% identical, about 70% identical, about 80% identical, about 90% identical, about 95% identical, about 98% identical, or about 99% identical to one another. Conservation of sequence may apply to the entire length of an oligonucleotide or polypeptide or may apply to a portion, region or feature thereof.

In some embodiments, conserved sequences are not contiguous. Those skilled in the art are able to appreciate how to achieve alignment when gaps in contiguous alignment are present between sequences, and to align corresponding residues not withstanding insertions or deletions present.

Delivery: As used herein, “delivery” refers to the act or manner of delivering a parvovirus e.g., AAV compound, substance, entity, moiety, cargo or payload to a target. Such target may be a cell, tissue, organ, organism, or system (whether biological or production).

Delivery Agent: As used herein, “delivery agent” refers to any agent which facilitates, at least in part, the delivery of one or more substances (including, but not limited to a compounds and/or compositions of the present disclosure, e.g., viral particles or AAV vectors) to targeted cells.

Delivery route: As used herein, the term “delivery route” and the synonymous term “administration route” refers to any of the different methods for providing a therapeutic agent to a subject. Routes of administration are generally classified by the location at which the substance is applied and may also be classified based on where the target of action is. Examples include, but are not limited to: intravenous administration, subcutaneous administration, oral administration, parenteral administration, enteral administration, topical administration, sublingual administration, inhalation administration, and injection administration, or other routes of administration described herein.

Effective amount: As used herein, the term “effective amount” of an agent is that amount sufficient to effect beneficial or desired results, for example, upon single or multiple dose administration to a subject or a cell, in curing, alleviating, relieving or improving one or more symptoms of a disorder and, as such, an “effective amount” depends upon the context in which it is being applied. For example, in the context of administering an agent that treats a tau-related disorder, an effective amount of an agent is, for example, an amount sufficient to achieve treatment, as defined herein, of a tau-related disorder as compared to the response obtained without administration of the agent.

Epitope: As used herein, the term “epitope” refers to the moieties of an antigen that specifically interact with an antibody molecule. Such moieties, also referred to herein as epitopic determinants, typically comprise, or are part of, elements such as amino acid side chains or sugar side chains. An epitopic determinant can be defined by methods known in the art or disclosed herein, e.g., by crystallography or by hydrogen-deuterium exchange. At least one or some of the moieties on the antibody molecule that specifically interact with an epitopic determinant are typically located in a CDR(s). Typically, an epitope has a specific three dimensional structural characteristics. Typically, an epitope has specific charge characteristics. Some epitopes are linear epitopes while others are conformational epitopes.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; (4) folding of a polypeptide or protein; and/or (5) post-translational modification of a polypeptide or protein.

Excipient: As used herein, the term “excipient” refers to an inactive substance that serves as the vehicle or medium for an active pharmaceutical agent or other active substance.

Formulation: As used herein, a “formulation” includes at least a compound and/or composition of the present disclosure (e.g., a vector, AAV particle, etc.) and a delivery agent.

Fragment: A “fragment,” as used herein, refers to a contiguous portion of a whole. For example, fragments of proteins may comprise polypeptides obtained by digesting full-length protein isolated from cultured cells. In some embodiments, a fragment of a protein includes at least 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, 250 or more amino acids. A fragment may also refer to a truncation (e.g., an N-terminal and/or C-terminal truncation) of a protein or a truncation (e.g., at the 5′ and/or 3′ end) of a nucleic acid. A protein fragment may be obtained by expression of a truncated nucleic acid, such that the nucleic acid encodes a portion of the full-length protein.

Fully human: As used herein, the term “fully human” refers to an immunoglobulin, such as an antibody or antibody fragment, where the whole molecule is of human origin or consists of an amino acid sequence identical to a human form of the antibody or immunoglobulin.

Homology: As used herein, the term “homology” refers to the overall relatedness between polymeric molecules, e.g. between nucleic acid molecules (e.g. DNA molecules and/or RNA molecules) and/or between polypeptide molecules. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical or similar. The term “homologous” necessarily refers to a comparison between at least two sequences (polynucleotide or polypeptide sequences). In accordance with the disclosure, two polynucleotide sequences are considered to be homologous if the polypeptides they encode are at least about 50%, 60%, 70%, 80%, 90%, 95%, or even 99% identical for at least one stretch of at least about 20 amino acids. In some embodiments, homologous polynucleotide sequences are characterized by the ability to encode a stretch of at least 4-5 uniquely specified amino acids. For polynucleotide sequences less than 60 nucleotides in length, homology is typically determined by the ability to encode a stretch of at least 4-5 uniquely specified amino acids. In accordance with the disclosure, two protein sequences are considered to be homologous if the proteins are at least about 50%, 60%, 70%, 80%, or 90% identical for at least one stretch of at least about 20 amino acids. In many embodiments, homologous protein may show a large overall degree of homology and a high degree of homology over at least one short stretch of at least 3, 4, 5, 6, 7, 8, 9, 10, 11, 12, 13, 14, 15, 16, 17, 18, 19, 20, 25, 30, 35, 40, 45, 50, or more amino acids. In many embodiments, homologous proteins share one or more characteristic sequence elements. As used herein, the term “characteristic sequence element” refers to a motif present in related proteins. In some embodiments, the presence of such motifs correlates with a particular activity (such as biological activity).

Humanized: As used herein, the term “humanized” refers to a non-human sequence of a polynucleotide or a polypeptide which has been altered to increase its similarity to a corresponding human sequence.

Identity: As used herein, the term “identity” refers to the overall relatedness between polymeric molecules, e.g., between oligonucleotide molecules (e.g. DNA molecules and/or RNA molecules) and/or between polypeptide molecules. Calculation of the percent identity of two polynucleotide sequences, for example, may be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference in its entirety. For example, the percent identity between two nucleotide sequences can be determined, for example using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package using an NWSgapdna.CMP matrix. Methods commonly employed to determine percent identity between sequences include, but are not limited to those disclosed in Carillo, H., and Lipman, D., SIAM J Applied Math., 48:1073 (1988); incorporated herein by reference in its entirety. Techniques for determining identity are codified in publicly available computer programs. Computer software to determine homology between two sequences include, but are not limited to, GCG program package, Devereux, J., et al., Nucleic Acids Research, 12(1), 387 (1984)), BLASTP, BLASTN, and FASTA Altschul, S. F. et al., J. Molecular Biol., 215, 403 (1990)).

Immunoglobulin variable domain sequence or variable domain: As used herein, an “immunoglobulin variable domain sequence” or “variable domain” refers to an amino acid sequence which can form the structure of an immunoglobulin variable domain. For example, the sequence may include all or part of the amino acid sequence of a naturally-occurring variable domain. For example, the sequence may or may not include one, two, or more N- or C-terminal amino acids, or may include other alterations that are compatible with formation of the protein structure.

Intrabody: As used herein, the term “intrabody” refers to a form of antibody that is not secreted from a cell in which it is produced, but instead targets one or more intracellular proteins. Intrabodies may be used to affect a multitude of cellular processes including, but not limited to intracellular trafficking, transcription, translation, metabolic processes, proliferative signaling, and cell division. In some embodiments, methods of the present disclosure may include intrabody-based therapies. In some such embodiments, variable domain sequences and/or CDR sequences disclosed herein may be incorporated into one or more constructs for intrabody-based therapy.

Isolated: As used herein, the term “isolated” refers to a substance or entity that is altered or removed from the natural state, e.g., altered or removed from at least some of component with which it is associated in the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell. Such polynucleotides could be part of a vector and/or such polynucleotides or polypeptides could be part of a composition, and still be isolated in that such vector or composition is not part of the environment in which it is found in nature. In some embodiments, an isolated nucleic acid is recombinant, e.g., incorporated into a vector.

miR binding site series: As used herein, the “miR binding site series” or the “miR binding site” includes an RNA sequence on the RNA transcript produced by transcribing the AAV vector genome. The “miR binding site series” or the “miR binding site” also includes the DNA sequence corresponding to the RNA sequence, in that they differ only by the T in DNA and the U in RNA. The reverse complement of such DNA is the coding sequence for the RNA sequence. That is, in some embodiments, in an expression cassette containing a DNA positive strand, the miR binding site sequence is the reverse complement of the miRNA to which it binds.

Modified: As used herein, the term “modified” refers to a changed state or structure of a molecule or entity as compared with a parent or reference molecule or entity. Molecules may be modified in many ways including chemically, structurally, and functionally. In some embodiments, compounds and/or compositions of the present disclosure are modified by the introduction of non-natural amino acids, or non-natural nucleotides.

Monoclonal antibody: As used herein, the term “monoclonal antibody” or “monoclonal antibody composition” as used herein refer to a preparation of antibody molecules of single molecular composition. A monoclonal antibody composition displays a single binding specificity and affinity for a particular epitope. A monoclonal antibody can be made by hybridoma technology or by methods that do not use hybridoma technology (e.g., recombinant methods). “Humanized” forms of non-human (for example, murine) antibodies are chimeric immunoglobulins, immunoglobulin chains or fragments thereof (such as Fv, Fab, Fab′, F(ab′)2 or other antigen-binding subsequences of antibodies) which contain minimal sequence derived from non-human immunoglobulin. For the most part, humanized antibodies and antibody fragments thereof are human immunoglobulins (recipient antibody or antibody fragment) in which residues from a complementary-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, a humanized antibody/antibody fragment can comprise residues which are found neither in the recipient antibody nor in the imported CDR or framework sequences. These modifications can further refine and optimize antibody or antibody fragment performance. In general, the humanized antibody or antibody fragment thereof will comprise substantially all of at least one, and typically two, variable domains, in which all or substantially all of the CDR regions correspond to those of a non-human immunoglobulin and all or a significant portion of the FR regions are those of a human immunoglobulin sequence. The humanized antibody or antibody fragment can also comprise at least a portion of an immunoglobulin constant region (Fc), typically that of a human immunoglobulin. For further details, see Jones et al., Nature, 321: 522-525, 1986; Reichmann et al., Nature, 332: 323-329, 1988; Presta, Curr. Op. Struct. Biol., 2: 593-596, 1992.

Mutation: As used herein, the term “mutation” refers to a change and/or alteration. In some embodiments, mutations may be changes and/or alterations to proteins (including peptides and polypeptides) and/or nucleic acids (including polynucleic acids). In some embodiments, mutations comprise changes and/or alterations to a protein and/or nucleic acid sequence. Such changes and/or alterations may comprise the addition, substitution and or deletion of one or more amino acids (in the case of proteins and/or peptides) and/or nucleotides (in the case of nucleic acids and or polynucleic acids). In embodiments wherein mutations comprise the addition and/or substitution of amino acids and/or nucleotides, such additions and/or substitutions may comprise 1 or more amino acid and/or nucleotide residues and may include modified amino acids and/or nucleotides. One or more mutations may result in a “mutant,” “derivative,” or “variant,” e.g., of a nucleic acid sequence or polypeptide or protein sequence.

Variant: The term “variant” refers to a polypeptide or polynucleotide that has an amino acid or a nucleotide sequence that is substantially identical, e.g., having at least 70%, 75%, 80%, 85%, 90%, 95% or 99% sequence identity to a reference sequence. In some embodiments, the variant is a functional variant.

Functional Variant: The term “functional variant” refers to a polypeptide variant or a polynucleotide variant that has at least one activity of the reference sequence.

Insertional Variant: “Insertional variants” when referring to polypeptides are those with one or more amino acids inserted, e.g., immediately adjacent or subsequent, to a position in an amino acid sequence. “Immediately adjacent” or “immediately subsequent” to an amino acid means connected to either the alpha-carboxy or alpha-amino functional group of the amino acid.

Monoclonal Antibody or Monoclonal Antibody Composition: The terms “monoclonal antibody” or “monoclonal antibody composition” as used herein refer to a preparation of antibody molecules of single molecular composition. A monoclonal antibody composition displays a single binding specificity and affinity for a particular epitope. A monoclonal antibody can be made by hybridoma technology or by methods that do not use hybridoma technology (e.g., recombinant methods). “Humanized” forms of non-human (for example, murine) antibodies are chimeric immunoglobulins, immunoglobulin chains or fragments thereof (such as Fv, Fab, Fab′, F(ab′)2 or other antigen-binding subsequences of antibodies) which contain minimal sequence derived from non-human immunoglobulin. For the most part, humanized antibodies and antibody fragments thereof are human immunoglobulins (recipient antibody or antibody fragment) in which residues from a complementary-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, a humanized antibody/antibody fragment can comprise residues which are found neither in the recipient antibody nor in the imported CDR or framework sequences. These modifications can further refine and optimize antibody or antibody fragment performance. In general, the humanized antibody or antibody fragment thereof will comprise substantially all of at least one, and typically two, variable domains, in which all or substantially all of the CDR regions correspond to those of a non-human immunoglobulin and all or a significant portion of the FR regions are those of a human immunoglobulin sequence. The humanized antibody or antibody fragment can also comprise at least a portion of an immunoglobulin constant region (Fc), typically that of a human immunoglobulin. For further details, see Jones et al., Nature, 321: 522-525, 1986; Reichmann et al., Nature, 332: 323-329, 1988; Presta, Curr. Op. Struct. Biol., 2: 593-596, 1992.

Multibody or Multispecific Antibody: As used herein, the term ““multibody” or “multispecific antibody” refer to an antibody comprising a plurality of immunoglobulin variable domain sequences, wherein a first immunoglobulin variable domain sequence of the plurality has binding specificity for a first epitope and a second immunoglobulin variable domain sequence of the plurality has binding specificity for a second epitope. In some embodiments, the first and second epitopes are on the same antigen, e.g., the same protein (or subunit of a multimeric protein). In some embodiments, the first and second epitopes overlap or substantially overlap. In some embodiments, the first and second epitopes do not overlap or do not substantially overlap. In some embodiments, the first and second epitopes are on different antigens, e.g., different proteins (or different subunits of a multimeric protein). In some embodiments, a multispecific antibody molecule comprises a third, fourth or fifth immunoglobulin variable domain. In some embodiments, a multispecific antibody molecule is a bispecific antibody molecule, a trispecific antibody molecule, or tetraspecific antibody molecule.

Nucleic acid: As used herein, the terms “nucleic acid,” “polynucleotide,” and “oligonucleotide” refer to any nucleic acid polymers composed of either polydeoxyribonucleotides (containing 2-deoxy-D-ribose), or polyribonucleotides (containing D-ribose), or any other type of polynucleotide that is an N glycoside of a purine or pyrimidine base, or modified purine or pyrimidine bases. There is no intended distinction in length between the term “nucleic acid,” “polynucleotide,” and “oligonucleotide,” and these terms will be used interchangeably. These terms refer only to the primary structure of the molecule. Thus, these terms include double- and single-stranded DNA, as well as double- and single-stranded RNA.

Operably linked: As used herein, the phrase “operably linked” refers to a functional connection between two or more molecules, constructs, transcripts, entities, moieties or the like.

Particle: As used herein, a “particle” is a virus comprised of at least two components, a protein capsid and a polynucleotide sequence enclosed within the capsid.

Payload: As used herein, “payload” or “payload region” refers to one or more polynucleotides or polynucleotide regions encoded by or within a viral genome or an expression product of such polynucleotide or polynucleotide region, e.g., a transgene, a polynucleotide encoding a polypeptide.

Payload construct: As used herein, “payload construct” is one or more polynucleotide regions encoding or comprising a payload that is flanked on one or both sides by an inverted terminal repeat (ITR) sequence. The payload construct is a template that is replicated in a viral production cell to produce a viral genome.

Payload construct vector: As used herein, “payload construct vector” is a vector encoding or comprising a payload construct, and regulatory regions for replication and expression in bacterial cells. The payload construct vector may also comprise a component for viral expression in a viral replication cell.

Pharmaceutically acceptable: The phrase “pharmaceutically acceptable” is employed herein to refer to those compounds, materials, compositions, and/or dosage forms which are suitable for use in contact with the tissues of human beings and animals.

Pharmaceutically acceptable excipients: As used herein, the term “pharmaceutically acceptable excipient,” as used herein, refers to any ingredient other than active agents (e.g., as described herein) present in pharmaceutical compositions that can function as vehicles for suspending and/or dissolving active agents.

Pharmaceutically acceptable salts: Pharmaceutically acceptable salts of the compounds described herein are forms of the disclosed compounds wherein the acid or base moiety is in its salt form (e.g., as generated by reacting a free base group with a suitable organic acid). Examples of pharmaceutically acceptable salts include, but are not limited to, mineral or organic acid salts of basic residues such as amines; alkali or organic salts of acidic residues such as carboxylic acids; and the like. Lists of suitable salts are found in Remington's Pharmaceutical Sciences, 17^{th} ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977), the contents of each of which are incorporated herein by reference in their entirety.

Pharmaceutical Composition: As used herein, the term “pharmaceutical composition” or pharmaceutically acceptable composition” comprises AAV polynucleotides, AAV genomes, or AAV particle and one or more pharmaceutically acceptable excipients, solvents, adjuvants, and/or the like.

Polypeptide: As used herein, “polypeptide” means a polymer of amino acid residues (natural or unnatural) linked together most often by peptide bonds. The term, as used herein, refers to proteins, polypeptides, and peptides of any size, structure, or function. In some instances, the polypeptide encoded is smaller than about 50 amino acids and the polypeptide is then termed a peptide. If the polypeptide is a peptide, it will be at least about 2, 3, 4, or at least 5 amino acid residues long. Thus, polypeptides include gene products, naturally occurring polypeptides, synthetic polypeptides, homologs, orthologs, paralogs, fragments and other equivalents, variants, and analogs of the foregoing. A polypeptide may be a single molecule or may be a multi-molecular complex such as a dimer, trimer or tetramer. They may also comprise single chain or multichain polypeptides and may be associated or linked. The term polypeptide may also apply to amino acid polymers in which one or more amino acid residues are an artificial chemical analogue of a corresponding naturally occurring amino acid.

Polypeptide variant: The term “polypeptide variant” refers to molecules which differ in their amino acid sequence from a native or reference sequence. The amino acid sequence variants may possess substitutions, deletions, and/or insertions at certain positions within the amino acid sequence, as compared to a native or reference sequence. In some embodiments, a variant comprises a sequence having at least about 50%, at least about 80%, or at least about 90%, identical (homologous) to a native or a reference sequence.

Peptide: As used herein, “peptide” is less than or equal to 50 amino acids long, e.g., about 5, 10, 15, 20, 25, 30, 35, 40, 45, or 50 amino acids long.

Preventing: As used herein, the term “preventing” refers to partially or completely delaying onset of an infection, disease, disorder and/or condition; partially or completely delaying onset of one or more symptoms, features, or clinical manifestations of a particular infection, disease, disorder, and/or condition; partially or completely delaying onset of one or more symptoms, features, or manifestations of a particular infection, disease, disorder, and/or condition; partially or completely delaying progression from an infection, a particular disease, disorder and/or condition; and/or decreasing the risk of developing pathology associated with the infection, the disease, disorder, and/or condition.

Promoter: As used herein, the term “promoter” refers to a nucleic acid site to which a polymerase enzyme will bind to initiate transcription (DNA to RNA) or reverse transcription (RNA to DNA).

Purified: As used herein, the term “purify” means to make substantially pure or clear from one or more unwanted components, material defilement, admixture or imperfection. “Purified” refers to the state of being pure. “Purification” refers to the process of making pure. As used herein, a substance is “pure” if it is substantially free of (substantially isolated from) one or more components, e.g., one or more components found in a native context.

Region: As used herein, the term “region” refers to a zone or general area. In some embodiments, when referring to a protein or protein module, a region may comprise a linear sequence of amino acids along the protein or protein module or may comprise a three dimensional area, an epitope and/or a cluster of epitopes. In some embodiments, regions comprise terminal regions. As used herein, the term “terminal region” refers to regions located at the ends or termini of a given agent. When referring to proteins, terminal regions may comprise N- and/or C-termini. N-termini refer to the end of a protein comprising an amino acid with a free amino group. C-termini refer to the end of a protein comprising an amino acid with a free carboxyl group. N- and/or C-terminal regions may comprise the N- and/or C-termini as well as surrounding amino acids. In some embodiments, N- and/or C-terminal regions comprise from about 3 amino acids to about 30 amino acids, from about 5 amino acids to about 40 amino acids, from about 10 amino acids to about 50 amino acids, from about 20 amino acids to about 100 amino acids and/or at least 100 amino acids. In some embodiments, N-terminal regions may comprise any length of amino acids that includes the N-terminus, but does not include the C-terminus. In some embodiments, C-terminal regions may comprise any length of amino acids, which include the C-terminus, but do not comprise the N-terminus.

In some embodiments, when referring to a polynucleotide, a region may comprise a linear sequence of nucleic acids along the polynucleotide or may comprise a three dimensional area, secondary structure, or tertiary structure. In some embodiments, regions comprise terminal regions. As used herein, the term “terminal region” refers to regions located at the ends or termini of a given agent. When referring to polynucleotides, terminal regions may comprise 5′ and 3′ termini. 5′ termini refer to the end of a polynucleotide comprising a nucleic acid with a free phosphate group. 3′ termini refer to the end of a polynucleotide comprising a nucleic acid with a free hydroxyl group. 5′ and 3′ regions may there for comprise the 5′ and 3′ termini as well as surrounding nucleic acids. In some embodiments, 5′ and 3′ terminal regions comprise from about 9 nucleic acids to about 90 nucleic acids, from about 15 nucleic acids to about 120 nucleic acids, from about 30 nucleic acids to about 150 nucleic acids, from about 60 nucleic acids to about 300 nucleic acids and/or at least 300 nucleic acids. In some embodiments, 5′ regions may comprise any length of nucleic acids that includes the 5′ terminus, but does not include the 3′ terminus. In some embodiments, 3′ regions may comprise any length of nucleic acids, which include the 3′ terminus, but does not comprise the 5′ terminus.

RNA or RNA molecule: As used herein, the term “RNA” or “RNA molecule” or “ribonucleic acid molecule” refers to a polymer of ribonucleotides; the term “DNA” or “DNA molecule” or “deoxyribonucleic acid molecule” refers to a polymer of deoxyribonucleotides. DNA and RNA can be synthesized naturally, e.g., by DNA replication and transcription of DNA, respectively; or be chemically synthesized. DNA and RNA can be single-stranded (i.e., ssRNA or ssDNA, respectively) or multi-stranded (e.g., double stranded, i.e., dsRNA and dsDNA, respectively). The term “mRNA” or “messenger RNA”, as used herein, refers to a single stranded RNA that encodes the amino acid sequence of one or more polypeptide chains.

Sample: As used herein, the term “sample” refers to an aliquot or portion taken from a source and/or provided for analysis or processing. In some embodiments, a sample is from a biological source such as a tissue, cell or component part (e.g. a body fluid, including but not limited to blood, mucus, lymphatic fluid, synovial fluid, cerebrospinal fluid, saliva, amniotic fluid, amniotic cord blood, urine, vaginal fluid and semen).

ScFv: As used herein, the term “scFv” refers to a fusion protein comprising at least one antibody fragment comprising a variable region of a light chain and at least one antibody fragment comprising a variable region of a heavy chain, wherein the light and heavy chain variable regions are contiguously linked via a short flexible polypeptide linker, and capable of being expressed as a single chain polypeptide, and wherein the scFv retains the specificity of the intact antibody from which it is derived. Unless specified, as used herein an scFv may have the VL and VH variable regions in either order, for example, with respect to the N-terminal and C-terminal ends of the polypeptide, the scFv may comprise VL-linker-VH or may comprise VH-linker-VL. In some embodiments, the scFv may comprise the structure of NH2-VL-linker-VH—COOH or NH2-VH-linker-VL-COOH.

Serotype: As used herein, the term “serotype” refers to distinct variations in a capsid of an AAV based on surface antigens which allow epidemiologic classifications of the AAVs at the sub-species level.

Signal Sequences: As used herein, the phrase “signal sequences” refers to a sequence which can direct the transport or localization.

Similarity: As used herein, the term “similarity” refers to the overall relatedness between polymeric molecules, e.g. between polynucleotide molecules (e.g. DNA molecules and/or RNA molecules) and/or between polypeptide molecules. Calculation of percent similarity of polymeric molecules to one another can be performed in the same manner as a calculation of percent identity, except that calculation of percent similarity takes into account conservative substitutions as is understood in the art.

Spacer: As used herein, a “spacer” is generally any selected nucleic acid sequence of, e.g., 1, 2, 3, 4, 5, 6, 7, 8, 9, or 10 nucleotides in length, which is located between two or more consecutive miR binding site sequences.

Subject: As used herein, the term “subject” or “patient” refers to any organism to which a composition in accordance with the disclosure may be administered, e.g., for experimental, diagnostic, prophylactic, and/or therapeutic purposes. Similarly, “subject” or “patient” refers to an organism who may seek, who may require, who is receiving, or who will receive treatment or who is under care by a trained professional for a particular disease or condition. Typical subjects include animals (e.g., mammals such as mice, rats, rabbits, non-human primates, and humans). In certain embodiments, a subject or patient may be susceptible to or suspected of having a tau-related disorder. In certain embodiments, a subject or patient may be diagnosed with Alzheimer's disease (AD), frontotemporal dementia and parkinsonism linked to chromosome 17 (FTDP-17), frontotemporal lobar degeneration (FTLD), frontotemporal dementia (FTD), chronic traumatic encephalopathy (CTE), progressive Supranuclear Palsy (PSP), Down's syndrome, Pick's disease, corticobasal degeneration (CBD), corticobasal syndrome, amyotrophic lateral sclerosis (ALS), prion diseases, Creutzfeldt-Jakob disease (CJD), multiple system atrophy, tangle-only dementia, or progressive subcortical gliosis.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Suffering from: An individual who is “suffering from” a disease, disorder, and/or condition has been diagnosed with or displays one or more symptoms of a disease, disorder, and/or condition.

Susceptible to: An individual who is “susceptible to” a disease, disorder, and/or condition has not been diagnosed with and/or may not exhibit symptoms of the disease, disorder, and/or condition but harbors a propensity to develop a disease or its symptoms. In some embodiments, an individual who is susceptible to a disease, disorder, and/or condition (for example, cancer) may be characterized by one or more of the following: (1) a genetic mutation associated with development of the disease, disorder, and/or condition; (2) a genetic polymorphism associated with development of the disease, disorder, and/or condition; (3) increased and/or decreased expression and/or activity of a protein and/or nucleic acid associated with the disease, disorder, and/or condition; (4) habits and/or lifestyles associated with development of the disease, disorder, and/or condition; (5) a family history of the disease, disorder, and/or condition; and (6) exposure to and/or infection with a microbe associated with development of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Synthetic: The term “synthetic” means produced, prepared, and/or manufactured by the hand of man. Synthesis of polynucleotides or polypeptides or other molecules of the present disclosure may be chemical or enzymatic.

Targeting: As used herein, “targeting” means the process of design and selection of nucleic acid sequence that will hybridize to a target nucleic acid and induce a desired effect.

Targeted Cells: As used herein, “target cells” or “targeted cells” refers to any one or more cells of interest. The cells may be found in vitro, in vivo, in situ or in the tissue or organ of an organism. The organism may be an animal, a mammal, a human and/or a patient. The target cells may be CNS cells or cells in CNS tissue.

Therapeutic Agent: The term “therapeutic agent” refers to any agent that, when administered to a subject has a therapeutic, diagnostic, and/or prophylactic effect and/or elicits a desired biological and/or pharmacological effect.

Therapeutically effective amount: As used herein, the term “therapeutically effective amount” means an amount of an agent to be delivered (e.g., nucleic acid, drug, therapeutic agent, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition. In some embodiments, a therapeutically effective amount is provided in a single dose. In some embodiments, a therapeutically effective amount is administered in a dosage regimen comprising a plurality of doses. Those skilled in the art will appreciate that in some embodiments, a unit dosage form may be considered to comprise a therapeutically effective amount of a particular agent or entity if it comprises an amount that is effective when administered as part of such a dosage regimen.

Thoracic Region: As used herein, a “thoracic region” refers to a region of the spinal cord comprising the thoracic vertebrae T1, T2, T3, T4, T5, T6, T7, T8, T9, T10, T11, and T12.

Treating: As used herein, the term “treating” refers to partially or completely alleviating, ameliorating, improving, relieving, reversing, delaying onset of, inhibiting progression of, reducing severity of, and/or reducing incidence of one or more symptoms or features of a particular infection, disease, disorder, and/or condition. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

Unmodified: As used herein, “unmodified” refers to any substance, compound or molecule prior to being changed in any way. Unmodified may, but does not always, refer to the wild-type or native form of a biomolecule or entity. Molecules or entities may undergo a series of modifications whereby each modified product may serve as the “unmodified” starting molecule or entity for a subsequent modification.

Vector: As used herein, a “vector” is any molecule or moiety which transports, transduces or otherwise acts as a carrier of a heterologous molecule. Vectors of the present disclosure may be produced recombinantly and may be based on and/or may comprise adeno-associated virus (AAV) parent or reference sequence(s). Such parent or reference AAV sequences may serve as an original, second, third or subsequent sequence for engineering vectors. In non-limiting examples, such parent or reference AAV sequences may comprise any one or more of the following sequences: a polynucleotide sequence encoding a tau binding protein (e.g., an anti-tau antibody), having a sequence that may be wild-type or modified from wild-type and which sequence may encode full-length or partial sequence of a protein, protein domain; a polynucleotide encoding an antibody, or antigen-binding portion thereof, which binds to tau and variants thereof, having a sequence that may be wild-type or modified from wild-type; and a transgene encoding an antibody, or antigen-binding portion thereof, which binds to tau and variants thereof that may or may not be modified from a wild-type sequence.

Viral construct vector. As used herein, a “viral construct vector” is a vector which comprises one or more polynucleotide regions encoding or comprising Rep and or Cap protein. A viral construct vector may also comprise one or more polynucleotide region encoding or comprising components for viral expression in a viral replication cell.

Viral genome: As used herein, a “viral genome” or “vector genome” is a polynucleotide comprising at least one inverted terminal repeat (ITR) and at least one encoded payload. A viral genome encodes at least one copy of the payload.

### EXAMPLES

### Example 1. NHP High-Throughput Screen of TRACER AAV Libraries

A TRACER based method as described in WO2020072683, the contents of which are herein incorporated by reference in their entirety, was adapted for use in non-human primates (NHP), as described in WO 2021/202651 and WO2021/230987, the contents of which are herein incorporated by reference in their entirety. An orthogonal evolution approach was combined with a high throughput screening by NGS in NHP as described in WO 2021/202651 and WO2021/230987, the contents of which are herein incorporated by reference in their entirety. Briefly, AAV9/AAV5 starting libraries, driven by synapsin or GFAP promoters were administered to non-human primate (NHP) intravenously for in vivo AAV selection (biopanning), performed iteratively. All libraries were injected intravenously at a dose of 1e14VG per animal (approximately 3e13 VG/kg). Orthogonally, biopanning was conducted in hBMVEC cells using the same starting libraries. In the second round of biopanning in NHP, only libraries driven by the synapsin promoter were used. After a period, (e.g., 1 month) RNA was extracted from nervous tissue, e.g., brain and spinal cord. Following RNA recovery and RT-PCR amplification, a systematic NGS enrichment analysis was performed, and the peptides shown in Table 20 identified. Capsid enrichment ratio, including calculating the ratio of, e.g., P2/P1 reads and comparison to a benchmark (e.g., AAV9) was evaluated.

Candidate library enrichment data in P3 NHP brain for the peptides identified, over benchmark AAV9, are shown in Table 20. Data are provided as fold enrichment. Fifty-one variants showed greater than 10-fold enrichment over AAV9. Variants with 0.0 enrichment over AAV9 are not included in Table 20.

TABLE 20
NHP NGS AAV9 Enrichment

| | | Fold | | | Fold |
|---|---|---|---|---|---|
| | | enrich- | | | enrich- |
| | | ment | | | ment |
| Peptide | SEQ | over | Peptide | SEQ | over |
| Sequence | ID | AAV9 | Sequence | ID | AAV9 |
| PLNGAVHLYA | 1139 | 473.7 | AQAGEQSTRL | 1166 | 16.1 |
| AQARDSPKGW | 1140 | 214 | AQASNDVGRA | 1167 | 15.4 |
| LTNGAVRDRP | 1141 | 134.4 | AQATFTASEY | 1168 | 15.3 |
| VQAFTHDSRG | 1142 | 88.6 | AKAHAGTIYS | 1169 | 14.9 |
| AQAYSTDVRM | 1143 | 84.8 | AQARTIDQCC | 1170 | 14.8 |
| AQAYSTDVRI | 1144 | 83.8 | AQEYNSNPKA | 1171 | 14.5 |
| AQAFTAAERM | 1145 | 74.9 | AQVVDNSTHA | 1172 | 14.5 |
| AQTHLQIGVA | 1146 | 54.6 | AQATLSVPLK | 1173 | 14.4 |
| AQSNAVLSLA | 1147 | 51.6 | AQIVMNSLKA | 1174 | 12.5 |
| AQAYSTDERM | 1148 | 41.4 | AQATMSQTMA | 1175 | 12.5 |
| AQAYSTDVRL | 1149 | 31.7 | AQALTQDERW | 1176 | 12 |
| AQATVSTLRM | 1150 | 31.5 | AQAQLSTLRP | 1177 | 11.6 |
| AQAYSTDERK | 1151 | 31.2 | AQVVMGISVA | 1178 | 11.4 |
| AQAYSTDMRM | 1152 | 30.4 | AQAYTTDVRM | 1179 | 11.4 |
| VVNGAVLHVA | 1153 | 29.8 | AQHIDSMRPP | 1180 | 11.3 |
| AQAYSTDVTM | 1154 | 29.7 | AQASTGTLRL | 1181 | 11.1 |
| AQAHLQIGVA | 1155 | 23 | AQHRALDYYA | 1182 | 11 |
| FLDPAVSSKA | 1156 | 22.6 | AQARESPRGL | 1183 | 10.9 |
| AQAYVSTLRM | 1157 | 21.9 | AQALLAGTRV | 1184 | 10.7 |
| AQAQTGPPLK | 1158 | 20.1 | TKIQAVPWNA | 1185 | 10.7 |
| EQASRLPTPG | 1159 | 20 | AQASLSSTRP | 1186 | 10.6 |
| AQASVSTMRM | 1160 | 19.7 | AQAMGSRSDQ | 1187 | 10.4 |
| TDYSAVRLGA | 1161 | 18 | AQAAQGTYRG | 1188 | 10.3 |
| TQAYSTDVRM | 1162 | 17.9 | SQENAVFSKA | 1189 | 10.3 |
| AQALPSNERL | 1163 | 17.4 | AQAYGLPKGP | 1190 | 8.4 |
| AQAYSTDVRT | 1164 | 16.4 | GGTLAVVSLA | 1191 | 6.9 |
| AQSSLPEMVA | 1165 | 16.2 | AQAYVSSVKM | 1192 | 5.2 |

A subset of the peptide variants from the NHP biopanning showed a very strong and consistent enrichment over AAV9 and PHP.B controls. Further, the peptide of SEQ ID NO: 3648 not only showed a strong enrichment over AAV9 in the brain, but also in the spinal cord, as it led to a 125.6 fold enrichment over AAV9 in the spinal cord. Following the removal of the least reliable variants, a set of 22 variants with enrichment factors ranging from 7-fold to >400-fold over AAV9 was identified. These were cross-referenced to a non-synthetic PCR-amplified library screened in parallel and 12 candidates showed reliable enrichment and high consistency in both assays. Of these, 5 candidates with the highest enrichment scores in both assays and the highest consistency across animals and tissues were retained for individual evaluation. Candidate capsids were labeled TTD-001, TTD-002, TTD-003, TTD-004 and TTD-005 as shown in Table 3.

After 3 rounds of screening of AAV9 peptide insertion library in NHP, many capsids outperformed their parental capsid AAV9 in penetration of the blood brain barrier (BBB). Some of the capsids comprising a peptide showed high enrichment scores and high consistency both across different brain tissue samples from the same animal and across different animals. Consistency in both NNK and NNM codons was also observed. 22 capsid variants exhibited enrichment factors ranging from 7-fold to >400-fold over AAV9 in the brain tissues. A majority of these variants also demonstrated high enrichment factors up to 125-fold over AAV9 in the spinal cord. Of these, 5 candidates with diverse inserted sequences were selected for further evaluation as individual capsids.

### Example 2. Individual Capsid Characterization

The goal of these experiments was to determine the transduction level and the spatial distribution of each of the 5 capsid candidates selected from the study described in Example 1 relative to AAV9 following intravascular infusion in NHPs (cynomolgus macaque). The 5 selected capsid candidates were TTD-001 (SEQ ID NO: 3623 and 3636, comprising SEQ ID NO: 3648), TTD-002 (SEQ ID NO: 3624, 3625, and 3637, comprising SEQ ID NO: 3649), TTD-003 (SEQ ID NO: 3626 and 3638, comprising SEQ ID NO: 3650), TTD-004 (SEQ ID NO: 3627 and 3639, comprising SEQ ID NO: 3651) and TTD-005 (SEQ ID NO: 3628 and 3640, comprising SEQ ID NO: 3652) as outlined in Table 3.

AAV particles were generated with each of these 5 capsids encapsulating a transgene encoding a payload fused to an HA tag (payload-HA) and driven by a full-length CMV/chicken beta actin promoter by triple transfection in HEK293T cells and formulated in a pharmaceutically acceptable solution. Each test capsid and AAV9 control were tested by intravenously providing two (2) NHP females the AAV particle formulation at a dose of 2e13 VG/kg. The in-life period was 14 days and then a battery of CNS and peripheral tissues were collected for quantification of transgene mRNA, transgene protein and viral DNA (biodistribution). Samples were also collected, fixed and paraffin embedded for immunohistochemical staining.

In a first pass screening of RNA quantification by qRT-PCR and RT-ddPCR, total RNA was extracted from 3-mm punches from various areas of the brain (cortex, striatum, hippocampus, cerebellum), spinal cord sections, liver and heart, and analyzed by qRT-PCR using a proprietary Taqman set specific for the synthetic CAG exon-exon junction. Cynomolgus TBP (TATA box-binding protein) was used as a housekeeping gene.

TRACER capsids showed an increase in RNA expression in all brain regions relative to AAV9 in at least one animal. The highest and most consistent increase in brain transduction was observed with capsids TTD-003 and TTD-004 (8- to 200-fold depending in various anatomical locations). In this initial screening TTD-001 was not assessed due to staggered animal dosing. An approximate 10- to 12-fold increase was consistently observed in whole brain slices (equivalent to an average of multiple regions), which was consistent with the values indicated in a next-generation sequencing (NGS) assay. In order to increase data robustness, droplet digital RT-PCR (ddPCR) was performed in parallel to qRT-PCR and confirmed the trends indicated by the qPCR data.

Interestingly, RNA quantification performed in the spinal cord and dorsal root ganglia indicated important differences between the capsid variants. The spinal cord transduction profile was consistent with the brain, with a strong and consistent increase with TTD-003 and TTD-004 capsids, but interestingly the DRG transduction suggested a substantial detargeting of the TTD-004 capsid, whereas the TTD-003 capsid showed a strongly increased RNA expression.

Total DNA was extracted from the same brain tissues as RNA, and biodistribution was measured by ddPCR using a Tagman set specific for the CMV promoter sequence. The RNAseP gene was used as a copy number reference. Vector genome (VG) per cell values were determined both by qPCR and ddPCR. Increased biodistribution was observed for the TTD-004 capsid in most brain regions, but surprisingly none of the other candidates showed a significant increase by comparison with AAV9. This apparent contradiction with the RNA quantification data could suggest that some capsids may present improved properties over AAV9 in post-attachment mechanisms rather than strict vector translocation in CNS parenchyma. Interestingly, DNA analysis confirmed the substantial detargeting of TTD-004 capsid from the DRG.

To further explore the behavior of capsid variant TTD-004, viral genome (VG) quantification was completed from tissues collected from heart atrium, heart ventricle, quadriceps muscle, liver (left and right) and diaphragm and compared to vector genome presence as delivered by AAV9 in the same tissues.

For TTD-003 and TTD-004 initial immunohistochemical analyses demonstrated the presence of payload-HA to a greater extent than seen with AAV9 delivery in cerebellar tissue, including in the dentate nucleus. Immunohistochemistry confirmed the de-targeting of the dorsal root ganglia for capsid variant TTD-004 as compared to TTD-003 and AAV9.

Data for each of the variants as described above were compiled as an average mRNA (fold over TBP) or DNA (VG per cell) quantification per capsid variant per tissue as shown in Table 21 below.

TABLE 21
Characterization of exemplary capsid variants

| Measure | Tissue | AAV9 | TTD-001 | TTD-002 | TTD-003 | TTD-004 | TTD-005 |
|---|---|---|---|---|---|---|---|
| mRNA | Frontal | 0.000325065 | 2.7232575 | 0.000768179 | 0.006268831 | 0.007076252 | 0.002204024 |
| | Cortex | | | | | | |
| mRNA | Sensory | 0.001486245 | 3.400055 | 0.00417739 | 0.006788644 | 0.010976612 | 0.004139604 |
| | Cortex | | | | | | |
| mRNA | Motor | 0.00063318 | 9.00819 | 0.001050247 | 0.009954825 | 0.010522399 | 0.002942249 |
| | Cortex | | | | | | |
| mRNA | Putamen | 0.000612759 | 3.557205 | 0.001395549 | 0.011832671 | 0.011476176 | 0.001150153 |
| mRNA | Thalamus | 0.002610992 | 2.863635 | 0.013937891 | 0.101411445 | 0.07565653 | 0.01100289 |
| mRNA | Cerebellar | 0.00133497 | 1.3439 | 0.008517779 | 0.006396677 | 0.012964181 | 0.004382119 |
| | Cortex | | | | | | |
| mRNA | Dentate | 0.001364954 | 0.963955 | — | — | — | — |
| | Nucleus | | | | | | |
| mRNA | Caudate | 0.000352281 | 1.3026 | — | 0.003259804 | 0.00634117 | — |
| mRNA | Hippocampus | 0.000311824 | 0.407015 | — | — | — | — |
| mRNA | SC-cervical | 0.012205449 | 11.877762 | 0.022004264 | 0.026994764 | 0.088316491 | 0.005773054 |
| mRNA | SC-Thoracic | 0.048833465 | 2.9974295 | 0.004360318 | 0.035118928 | 0.020543776 | 0.005629959 |
| mRNA | SC-Lumbar | 0.029887407 | 7.969603 | 0.056231995 | 0.016033388 | 0.047713563 | 0.026324154 |
| mRNA | DRG- | 0.74570895 | 9.274951 | 0.007897714 | 2.47872652 | 0.280868887 | 0.008122233 |
| | cervical | | | | | | |
| mRNA | DRG- | 0.5559061 | 5.22606 | 0.006456564 | 8.721845271 | 0.104701895 | — |
| | Thoracic | | | | | | |
| mRNA | DRG- | 1.089758 | 17.308436 | 0.008247771 | 2.271300217 | 0.426704698 | 0.119974244 |
| | Lumbar | | | | | | |
| mRNA | Lung | 0.004807149 | 0.000546842 | — | — | 0.013744781 | — |
| mRNA | Pancreas | 1 | — | — | — | — | — |
| mRNA | Colon | 0.017962678 | 0.005041385 | — | — | 0.183862903 | — |
| mRNA | Kidney | 0.043825993 | 0.006649157 | — | — | 0.041234576 | — |
| mRNA | Liver | 0.674478605 | 0.253188648 | — | — | 2.578654807 | — |
| mRNA | Adrenal | — | — | — | — | — | — |
| mRNA | Spleen | 0.014066875 | 0.000955981 | — | — | 0.013435626 | — |
| mRNA | Heart | 1.323389668 | 0.132477314 | — | — | 5.587929805 | — |
| mRNA | Quadriceps | 0.116623509 | — | — | — | 4.527799743 | — |
| mRNA | Diaphragm | 0.250001109 | — | — | — | 1.936435215 | — |
| DNA | Frontal | 0.07713 | 2.104843 | 0.10252 | 0.068367 | 0.380429 | 0.1257545 |
| | Cortex | | | | | | |
| DNA | Sensory | 0.093003 | 2.679886 | 0.07443 | 0.034016 | 0.2670975 | 0.132503 |
| | Cortex | | | | | | |
| DNA | Motor | 0.08796 | 4.3437625 | 0.0913085 | 0.094401 | 0.318999 | 0.1110695 |
| | Cortex | | | | | | |
| DNA | Putamen | 0.0581365 | 3.07904 | 0.12326 | 0.1497635 | 0.2731175 | 0.0715295 |
| DNA | Thalamus | 0.0524055 | 2.076863 | 0.0664225 | 0.090511 | 0.214999 | 0.086863 |
| DNA | Cerebellar | 0.014238 | 0.186361 | 0.0092915 | 0.009578 | 0.0356345 | 0.0128655 |
| | Cortex | | | | | | |
| DNA | Dentate | 0.025042 | 0.1861975 | 0.210238 | 0.041906 | 0.106107 | 0.055287 |
| | Nucleus | | | | | | |
| DNA | Caudate | 0.079294 | 3.9433175 | — | 0.0529005 | 0.2451035 | — |
| DNA | Hippocampus | 0.095436 | 1.760891 | 0.205433 | 0.368645 | 1.335324 | 0.432829 |
| DNA | SC-cervical | 0.0376 | 1.143863 | 0.061085 | 0.061535 | 0.07573 | 0.05885 |
| DNA | SC-Thoracic | 0.02692 | 0.933734 | 0.025955 | 0.05011 | 0.064915 | 0.0355 |
| DNA | SC-Lumbar | 0.03615 | 0.992728 | 0.019125 | 0.034175 | 0.085165 | 0.051475 |
| DNA | DRG- | 0.0765 | 0.14319 | 0.08196 | 0.13722 | 0.04115 | 0.071625 |
| | cervical | | | | | | |
| DNA | DRG- | 0.165865 | 0.172363 | 0.07202 | 0.133455 | 0.04444 | 0.03139 |
| | Thoracic | | | | | | |
| DNA | DRG- | 0.218725 | 0.385712 | 0.146115 | 0.153205 | 0.032875 | 0.12034 |
| | Lumbar | | | | | | |
| DNA | Lung | 1.085639916 | 3.72 | 0.958576278 | 0.700015423 | 1.22442329 | 0.919823152 |
| DNA | Pancreas | 0.256670617 | 20.535 | 0.320558325 | 0.240633195 | 0.067860607 | 0.004802583 |
| DNA | Colon | 0.053867646 | 3.405 | 1.179065405 | 0.348969617 | 0.116867365 | 0.015288464 |
| DNA | Kidney | 0.896656371 | 26.635 | 4.861362029 | 0.532746958 | 0.386522209 | 7.973793288 |
| DNA | Liver | 207.332334 | 217.64 | 111.910319 | 193.8349405 | 448.5980021 | 213.0317219 |
| DNA | Adrenal | 1.647725996 | 0.69 | 1.561129869 | 1.871878 | 1.269473156 | 0.847293047 |
| DNA | Spleen | 14.93815481 | 20.43565 | 51.70294001 | 22.79095714 | 6.514778227 | 45.91987284 |
| DNA | Heart | 2.012377817 | 14.49 | 0.757528914 | 1.780956673 | 3.814571986 | 0.44694144 |
| DNA | Quadriceps | 0.724278943 | 1.285 | 0.476250457 | 1.366015493 | 5.611203726 | 0.646197937 |
| DNA | Diaphragm | — | 1.06 | — | — | — | — |

When calculated as fold over AAV9 the data were as shown in Table 22 below.

TABLE 22
Characterization of exemplary capsid variants

| Measure | Tissue | AAV9 | TTD-001 | TTD-002 | TTD-003 | TTD-004 | TTD-005 |
|---|---|---|---|---|---|---|---|
| mRNA | Frontal | 1.0 | 8378 | 2.4 | 19.3 | 21.8 | 6.8 |
| | Cortex | | | | | | |
| mRNA | Sensory | 1.0 | 2288 | 2.8 | 4.6 | 7.4 | 2.8 |
| | Cortex | | | | | | |
| mRNA | Motor Cortex | 1.0 | 14227 | 1.7 | 15.7 | 16.6 | 4.6 |
| mRNA | Putamen | 1.0 | 5805 | 2.3 | 19.3 | 18.7 | 1.9 |
| mRNA | Thalamus | 1.0 | 1097 | 5.3 | 38.8 | 29.0 | 4.2 |
| mRNA | Cerebellar | 1.0 | 1007 | 6.4 | 4.8 | 9.7 | 3.3 |
| | Cortex | | | | | | |
| mRNA | Dentate | 1.0 | 706 | — | — | — | — |
| | Nucleus | | | | | | |
| mRNA | Caudate | 1.0 | 3698 | — | — | — | — |
| mRNA | Hippocampus | 1.0 | 1305 | — | — | — | — |
| mRNA | SC-cervical | 1.0 | 973 | 1.8 | 2.2 | 7.2 | 0.5 |
| mRNA | SC-Thoracic | 1.0 | 61 | 0.1 | 0.7 | 0.4 | 0.1 |
| mRNA | SC-Lumbar | 1.0 | 267 | 1.9 | 0.5 | 1.6 | 0.9 |
| mRNA | DRG-cervical | 1.0 | 12 | 0.0 | 3.3 | 0.4 | 0.0 |
| mRNA | DRG-Thoracic | 1.0 | 9 | 0.0 | 15.7 | 0.2 | — |
| mRNA | DRG-Lumbar | 1.0 | 16 | 0.0 | 2.1 | 0.4 | 0.1 |
| mRNA | Lung | 1.0 | 0.11 | — | — | 2.9 | — |
| mRNA | Pancreas | — | — | — | — | — | — |
| mRNA | Colon | 1.0 | 0.28 | — | — | 10.2 | — |
| mRNA | Kidney | 1.0 | 0.15 | — | — | 0.9 | — |
| mRNA | Liver | 1.0 | 0.38 | — | — | 3.8 | — |
| mRNA | Adrenal | — | — | — | — | — | — |
| mRNA | Spleen | 1.0 | 0.07 | — | — | 1.0 | — |
| mRNA | Heart | 1.0 | 0.10 | — | — | 4.2 | — |
| mRNA | Quadriceps | 1.0 | — | — | — | 38.8 | — |
| mRNA | Diaphragm | 1.0 | — | — | — | 7.7 | — |
| DNA | Frontal | 1.0 | 27.29 | 1.3 | 0.9 | 4.9 | 1.6 |
| | Cortex | | | | | | |
| DNA | Sensory | 1.0 | 28.82 | 0.8 | 0.4 | 2.9 | 1.4 |
| | Cortex | | | | | | |
| DNA | Motor Cortex | 1.0 | 49.38 | 1.0 | 1.1 | 3.6 | 1.3 |
| DNA | Putamen | 1.0 | 52.96 | 2.1 | 2.6 | 4.7 | 1.2 |
| DNA | Thalamus | 1.0 | 39.63 | 1.3 | 1.7 | 4.1 | 1.7 |
| DNA | Cerebellar | 1.0 | 13.09 | 0.7 | 0.7 | 2.5 | 0.9 |
| | Cortex | | | | | | |
| DNA | Dentate | 1.0 | 7.44 | 8.4 | 1.7 | 4.2 | 2.2 |
| | Nucleus | | | | | | |
| DNA | Caudate | 1.0 | 49.73 | — | 0.7 | 3.1 | — |
| DNA | Hippocampus | 1.0 | 18.45 | 2.2 | 3.9 | 14.0 | 4.5 |
| DNA | SC-cervical | 1.0 | 30.42 | 1.6 | 1.6 | 2.0 | 1.6 |
| DNA | SC-Thoracic | 1.0 | 34.69 | 1.0 | 1.9 | 2.4 | 1.3 |
| DNA | SC-Lumbar | 1.0 | 27.46 | 0.5 | 0.9 | 2.4 | 1.4 |
| DNA | DRG-cervical | 1.0 | 1.87 | 1.1 | 1.8 | 0.5 | 0.9 |
| DNA | DRG-Thoracic | 1.0 | 1.04 | 0.4 | 0.8 | 0.3 | 0.2 |
| DNA | DRG-Lumbar | 1.0 | 1.76 | 0.7 | 0.7 | 0.2 | 0.6 |
| DNA | Lung | 1.0 | 3.43 | 0.9 | 0.6 | 1.1 | 0.8 |
| DNA | Pancreas | 1.0 | 80.01 | 1.2 | 0.9 | 0.3 | 0.0 |
| DNA | Colon | 1.0 | 63.21 | 21.9 | 6.5 | 2.2 | 0.3 |
| DNA | Kidney | 1.0 | 29.70 | 5.4 | 0.6 | 0.4 | 8.9 |
| DNA | Liver | 1.0 | 1.05 | 0.5 | 0.9 | 2.2 | 1.0 |
| DNA | Adrenal | 1.0 | 0.42 | 0.9 | 1.1 | 0.8 | 0.5 |
| DNA | Spleen | 1.0 | 1.37 | 3.5 | 1.5 | 0.4 | 3.1 |
| DNA | Heart | 1.0 | 7.20 | 0.4 | 0.9 | 1.9 | 0.2 |
| DNA | Quadriceps | 1.0 | 1.77 | 0.7 | 1.9 | 7.7 | 0.9 |
| DNA | Diaphragm | — | — | — | — | — | — |

Capsid variant TTD-001 showed greater than 5,000 fold increase in payload-HA levels delivered to the brain as compared to AAV9 and measured by qRT-PCR and normalized to TBP. In all CNS tissues measured, TTD-001 showed dramatically enhanced delivery of payload-HA as compared to AAV9. Immunohistochemistry of fixed brain tissues revealed dramatic transduction in both NHP tested by TTD-001 of the dentate nucleus, cerebellar cortex, cerebral cortex, brain stem, hippocampus, thalamus and putamen. AAV9 transduction of the dentate nucleus, cerebellar cortex, cerebral cortex, hippocampus, thalamus and putamen appeared negligible in comparison. TTD-001 therefore demonstrated broad and robust expression and distribution in the brain following intravenous administration in NHPs. In the dorsal root ganglia, both TTD-001 and AAV9 showed similar IHC patterns.

### Example 3. Maturation of TTD-001 Capsid in NHPs

This Example describes maturation of the TTD-001 (SEQ ID NO: 3623 (DNA) and 3636 (amino acid), comprising SEQ ID NO: 3648) capsid variant to further enhance its transduction and biodistribution in the central nervous system and evolve the AAV capsid variants further. Two approaches were used to mature the TTD-001 capsid sequence in order to randomize and mutate within and around the peptide insert comprised within loop VIII of the capsid variant. In the first maturation approach, sets of three contiguous amino acids were randomized across the mutagenesis region in the TTD-001 sequence, which spanned from position 587 to position 602, numbered according to SEQ ID NO: 3636. In the second maturation approach, mutagenic primers were used to introduce point mutations at a low frequency, scattered across the mutagenesis region in the TTD-001 sequences ranging from position 587 to position 602, numbered according to SEQ ID NO: 3636. AAV capsid variants arising from each maturation approach for TTD-001 were pooled together, for subsequent testing and characterization in NHPs (Macaca fascicularis and Callithrix jacchus).

The library of pooled matured AAV capsid variants generated from TTD-001 matured AAV capsid variant were injected into two cynomolgus macaques (Macaca fascicularis), two marmosets (Callithrix jacchus). After a period in life, the brains of the NHPs were isolated and RNA was extracted from three samples per NHP. Following RNA recovery and RT-PCR amplification, a systematic NGS enrichment analysis was performed to calculate the fold enrichment ratio relative to the corresponding TTD-001 control, and the peptides comprised within the variants were identified. The coefficient of variance (CV) was calculated for each peptide across the six samples, and those that had a CV value<1 were identified, as these were the peptides that were reliably detected in 5/6 or 6/6 of the brain samples isolated from the two NHPs. The average number of reads for each peptide across the samples investigated was also quantified. These TTD-001 matured capsid variants and their peptide sequences are provided in Table 23 (cynomolgus macaques (Macaca fascicularis)) and Table 24 (marmosets (Callithrix jacchus)).

As shown in Table 23, approximately 338 TTD-001 matured capsid variants demonstrated increased expression relative to the non-matured TTD-001 control, and several variants demonstrated greater than a two-fold enrichment relative to the non-matured TTD-001 control, in cynomolgus macaques (Macaca fascicularis). Also, across the peptides comprised within the TTD-001 matured capsid variants with the greatest fold-enrichment relative to the non-matured TTD-001 capsid in the brains of cynomolgus macaques, it was observed that the modifications in the variant sequences appeared in the C-terminal portion, specifically at residues corresponding to positions 593-595 of a reference sequence numbered according to the amino acid sequence of SEQ ID NO: 138. Additionally, 378 of the top peptides in Table 23 had an average read value of 1 or greater per sample, demonstrating that more functional capsid material was recovered, which could be indicative of less aggregation.

As shown in Table 24, many TTD-001 matured capsid variants demonstrated increased expression relative to both the AAV9 and the non-matured TTD-001 controls in the brains of marmosets. Approximately, 967 TTD-001 matured variants demonstrated increased expression relative to the non-matured TTD-001 control in the brain of marmosets, with 296 variants showing at least a 10-fold enrichment or greater relative to the non-matured TTD-001 control. Approximately, 850 TTD-001 matured variants demonstrated increased expression relative to AAV9 in the brain of marmosets, with 222 variants showing at least a 10-fold enrichment or greater relative to AAV9. With respect to those TTD-001 matured variants that demonstrated an increased expression in marmosets, it was observed that the majority comprised an amino acid other than Q at position 604 (e.g., Q604) numbered according to SEQ ID NO: 5, 8, or 3636 or at position 597 (Q597) numbered according to SEQ ID NO: 138 (e.g., an E, H, K, or P), such that they comprised the triplet “VEN,” “VHN,” “VKN,” or “VPN” at their C-terminus (corresponding to positions 596-598 of SEQ ID: 138 or positions 603-605 of SEQ ID NO: 5, 8, and 3636). Many of these TTD-001 matured variants also demonstrated an increased expression in the brain of cynomolgus macaques relative to AAV9 (Table 24), including the TTD-001 matured capsid variant comprising the sequence PLNGAVHLYAQAQLSPVKN (SEQ ID NO: 566) and the TTD-001 matured capsid variant comprising the sequence PLNGAVHLYAQAQTGWVPN (SEQ ID NO: 314).

The fold-change in expression relative to AAV9 and TTD-001 was also calculated in the DRG, heart, muscle (quadriceps), and liver for the TTD-001 matured variants in cynomolgus macaques. The fold-change in the DRG is shown in Table 24, with several variants showing decreased or comparable expression in the DRG relative to AAV9. These variants also demonstrated comparable or lower expression relative to AAV9 in the heart, muscle, and liver.

Taken together, these data demonstrate that following two maturation approaches, matured TTD-001 capsid variants with loop VIII modifications were generated with significantly enhanced CNS tropism in NHPs (cynomolgus macaques (Macaca fascicularis) and marmosets (Callithrix jacchus)), compared to the corresponding non-matured TTD-001 capsid variant, which already exhibited a significant fold enrichment over AAV9 in the NHP brain.

TABLE 23
NGS fold-enrichment of TTD-001 matured AAV capsid variants in the brain of NHPs

| | | | Fold | | | | | Fold | |
|---|---|---|---|---|---|---|---|---|---|
| | | | enrich- | | | | | enrich- | |
| Peptide | SEQ | | ment | | Peptide | SEQ | | ment | |
| Se- | ID | | over | Avg. | Se- | ID | | over | Avg. |
| quence | NO: | CV | TTD-001 | Reads | quence | NO: | CV | TTD-001 | Reads |
| QLNGAV | 139 | 0.206 | 5.617 | 0.09 | QLNGYV | 1138 | 0.513 | 0.626 | 0.09 |
| HLYAQA | | | | | HLYAQA | | | | |
| QLSPVQ | | | | | QTGWVQ | | | | |
| N | | | | | N | | | | |
| PLDGAV | 140 | 0.829 | 5.133 | 4.02 | QLNGAV | 639 | 0.845 | 0.626 | 0.09 |
| HLYAQP | | | | | HLYAQA | | | | |
| QTGWVQ | | | | | QTTGVQ | | | | |
| N | | | | | N | | | | |
| QLNGAV | 141 | 0.770 | 4.088 | 0.09 | PLNGAV | 640 | 0.602 | 0.625 | 39.96 |
| HLYAQA | | | | | HLYAQA | | | | |
| QTMSVQ | | | | | QTGFVQ | | | | |
| N | | | | | N | | | | |
| PLDSSV | 142 | 0.882 | 3.891 | 1.4 | PLNGAV | 641 | 0.536 | 0.625 | 42.62 |
| HLYAQA | | | | | HLYAQA | | | | |
| QTGWVQ | | | | | QSALVQ | | | | |
| N | | | | | N | | | | |
| PLNGAV | 143 | 0.879 | 3.879 | 5.47 | ALNGAV | 642 | 0.278 | 0.624 | 0.75 |
| HLYAQA | | | | | HLYAQA | | | | |
| QTTKVQ | | | | | QTGWDQ | | | | |
| N | | | | | N | | | | |
| PLDGAV | 144 | 0.586 | 3.835 | 11.08 | QLNGAV | 643 | 0.636 | 0.623 | 0.09 |
| HLYAQA | | | | | HLYAQA | | | | |
| QTGSVQ | | | | | QISGVQ | | | | |
| N | | | | | N | | | | |
| ALNGAV | 145 | 0.865 | 3.765 | 0.09 | QLNGAV | 644 | 0.520 | 0.623 | 0.14 |
| HLYAQA | | | | | HLYAQA | | | | |
| QTTSVQ | | | | | QRGSVQ | | | | |
| N | | | | | N | | | | |
| PLNGSV | 146 | 0.509 | 3.708 | 0.09 | PLNGAV | 645 | 0.270 | 0.622 | 0.19 |
| HLYAQA | | | | | HLYAQA | | | | |
| QTMSVQ | | | | | QTGWVK | | | | |
| N | | | | | T | | | | |
| QLNGAV | 147 | 0.310 | 3.638 | 0.14 | PLNGAV | 646 | 0.554 | 0.619 | 27.62 |
| HLYAQA | | | | | HLYAQA | | | | |
| QTSPVQ | | | | | QSQLVQ | | | | |
| N | | | | | N | | | | |
| PLNGAV | 148 | 0.547 | 3.634 | 5.61 | PLNGSV | 647 | 0.202 | 0.617 | 0.33 |
| HLYAQA | | | | | HLYAQA | | | | |
| QTMKVQ | | | | | QTGWGQ | | | | |
| N | | | | | N | | | | |
| PLNGAV | 149 | 0.397 | 3.550 | 21.87 | QLNGAV | 648 | 0.632 | 0.616 | 0.14 |
| HLYAQA | | | | | HLYAQA | | | | |
| QVAQVQ | | | | | QTGWVP | | | | |
| N | | | | | N | | | | |
| PLDGAV | 150 | 0.771 | 3.509 | 2.29 | PLNGAV | 649 | 0.305 | 0.615 | 0.33 |
| HLYAQA | | | | | HLYAKA | | | | |
| QTGGVQ | | | | | QTGWVK | | | | |
| Z | | | | | N | | | | |
| PLNGAV | 151 | 0.344 | 3.373 | 0.09 | PLNGAV | 650 | 0.164 | 0.615 | 0.09 |
| HLYAQA | | | | | HLYAHA | | | | |
| QTAWDQ | | | | | QTGWGQ | | | | |
| N | | | | | N | | | | |
| PLNGSV | 152 | 0.190 | 3.335 | 0.23 | ALNGAV | 651 | 0.330 | 0.613 | 0.61 |
| HLYAQA | | | | | HLYAQA | | | | |
| QTGWDQ | | | | | QTGWVQ | | | | |
| N | | | | | H | | | | |
| PLNGAV | 153 | 0.235 | 3.287 | 0.14 | PLNGSV | 652 | 0.585 | 0.612 | 0.14 |
| HLYAQA | | | | | HLYAQA | | | | |
| QTGSVQ | | | | | QIASVQ | | | | |
| H | | | | | N | | | | |
| PLNGAV | 154 | 0.564 | 3.259 | 4.25 | ALNGAV | 653 | 0.416 | 0.611 | 0.14 |
| HLYAQA | | | | | HLYAQA | | | | |
| QVKQVQ | | | | | QTAMVQ | | | | |
| N | | | | | N | | | | |
| PLNGAV | 155 | 0.763 | 3.239 | 24.54 | TLNGAV | 654 | 0.316 | 0.610 | 0.09 |
| HLYAQA | | | | | HLYAQA | | | | |
| QSAPVQ | | | | | QTMAVQ | | | | |
| N | | | | | N | | | | |
| PLNGAV | 156 | 0.591 | 3.156 | 6.73 | PHNGAV | 655 | 0.476 | 0.609 | 0.09 |
| HLYAQA | | | | | HLYAQA | | | | |
| QLSKVQ | | | | | QVSSVQ | | | | |
| N | | | | | N | | | | |
| PLNGAV | 157 | 0.587 | 3.107 | 31.03 | PLNGAV | 656 | 0.693 | 0.606 | 0.09 |
| HLYAQA | | | | | QLYAQA | | | | |
| QLAPVQ | | | | | QTAPVQ | | | | |
| N | | | | | N | | | | |
| PLNGAV | 158 | 0.416 | 3.061 | 15.33 | PLNGSV | 657 | 0.282 | 0.605 | 0.09 |
| HLYAQA | | | | | HLYAQA | | | | |
| QLAQVQ | | | | | QKGSVQ | | | | |
| N | | | | | N | | | | |
| QLNGAV | 159 | 0.295 | 3.051 | 0.09 | PLNGAV | 658 | 0.574 | 0.605 | 39.3 |
| HLYAQA | | | | | HLYAQA | | | | |
| QVASVQ | | | | | QLSAVQ | | | | |
| N | | | | | N | | | | |
| PLNGAV | 160 | 0.877 | 2.998 | 8.97 | PINGAV | 659 | 0.489 | 0.605 | 0.09 |
| HLYAQA | | | | | HLYAQA | | | | |
| QTAKVQ | | | | | QVSAVQ | | | | |
| N | | | | | N | | | | |
| PLNGAV | 161 | 0.678 | 2.971 | 3.27 | PLNGSV | 660 | 0.514 | 0.604 | 43.37 |
| HLYAQA | | | | | HLYAQA | | | | |
| QSAKVQ | | | | | QTGLVQ | | | | |
| N | | | | | N | | | | |
| PLNGAV | 162 | 0.095 | 2.962 | 0.09 | PLNGAV | 661 | 0.223 | 0.604 | 0.14 |
| HLYAQA | | | | | HIYAQA | | | | |
| QTGCFQ | | | | | QTGWFQ | | | | |
| N | | | | | N | | | | |
| PLNGAV | 163 | 0.705 | 2.958 | 5.51 | SLNGAV | 662 | 0.293 | 0.603 | 0.09 |
| HLYAQA | | | | | HLYAQA | | | | |
| QTQKVQ | | | | | QIASVQ | | | | |
| N | | | | | N | | | | |
| PLNGSV | 164 | 0.529 | 2.873 | 0.09 | PLNGAV | 663 | 0.220 | 0.601 | 0.14 |
| HLYAQA | | | | | HLYAQE | | | | |
| QTTSVQ | | | | | QTVSVQ | | | | |
| N | | | | | N | | | | |
| PLNGGV | 165 | 0.820 | 2.858 | 4.25 | PLNGAV | 664 | 0.737 | 0.601 | 0.09 |
| HLYAQA | | | | | HLYAQA | | | | |
| QTGRVQ | | | | | QTMQVQ | | | | |
| N | | | | | T | | | | |
| PLNGAV | 166 | 0.410 | 2.854 | 30.94 | HLNGAV | 665 | 0.338 | 0.600 | 56.78 |
| HLYAQA | | | | | HLYAQA | | | | |
| QTVAVQ | | | | | QTGWVQ | | | | |
| N | | | | | N | | | | |
| ALNGAV | 167 | 0.542 | 2.851 | 0.09 | PLNGAV | 666 | 0.400 | 0.600 | 24.02 |
| HLYAQA | | | | | HLYAQA | | | | |
| QSSPVQ | | | | | QTGQVQ | | | | |
| N | | | | | N | | | | |
| PLNGAV | 168 | 0.313 | 2.820 | 63.28 | QLNGAV | 667 | 0.596 | 0.599 | 0.19 |
| HLYAQA | | | | | HLYAQA | | | | |
| QLSPVQ | | | | | QTGWVQ | | | | |
| N | | | | | D | | | | |
| QLNGAV | 169 | 0.846 | 2.769 | 0.14 | PLNGAV | 668 | 0.526 | 0.599 | 0.09 |
| HLYAQA | | | | | HLYDQA | | | | |
| QTTSVQ | | | | | QVAQVQ | | | | |
| N | | | | | N | | | | |
| PLNGAV | 170 | 0.334 | 2.719 | 18.37 | PLNGAV | 669 | 0.515 | 0.598 | 0.19 |
| HLYAQA | | | | | HLYAQA | | | | |
| QTTQVQ | | | | | QTSPVK | | | | |
| N | | | | | N | | | | |
| PLNGAV | 171 | 0.523 | 2.707 | 25.84 | PLNGAI | 670 | 0.383 | 0.595 | 0.09 |
| HLYAQA | | | | | HLYAQA | | | | |
| QTAQVQ | | | | | QTSPVQ | | | | |
| N | | | | | N | | | | |
| QLNGAV | 172 | 0.195 | 2.682 | 0.09 | PLNGSV | 671 | 0.578 | 0.595 | 0.19 |
| HLYAQA | | | | | HLYAQA | | | | |
| QTVAVQ | | | | | QTAMVQ | | | | |
| N | | | | | N | | | | |
| PLNGAV | 173 | 0.682 | 2.652 | 3.27 | PLNGAV | 672 | 0.370 | 0.594 | 0.09 |
| HLYAQA | | | | | HLYAQA | | | | |
| QRIAVQ | | | | | QLSQVQ | | | | |
| N | | | | | T | | | | |
| PLNGAV | 174 | 0.340 | 2.634 | 17.34 | PLNGAV | 673 | 0.310 | 0.594 | 0.19 |
| HLYAQA | | | | | HLYAQA | | | | |
| QRASVQ | | | | | QTGSVL | | | | |
| N | | | | | N | | | | |
| PLNGAV | 175 | 0.400 | 2.620 | 45.19 | PLNGAI | 674 | 0.428 | 0.593 | 0.09 |
| HLYAQA | | | | | HLYAQA | | | | |
| QTTPVQ | | | | | QTASVQ | | | | |
| N | | | | | N | | | | |
| QLNGAV | 176 | 0.474 | 2.613 | 0.09 | PLNGAV | 675 | 0.785 | 0.592 | 6.68 |
| HLYAQA | | | | | HLYAQA | | | | |
| QLASVQ | | | | | QTNGVQ | | | | |
| N | | | | | N | | | | |
| PLNGAV | 177 | 0.528 | 2.574 | 39.86 | PLNGSV | 676 | 0.334 | 0.591 | 0.14 |
| HLYAQA | | | | | HLYAQA | | | | |
| QLTPVQ | | | | | QTGWVQ | | | | |
| N | | | | | Y | | | | |
| PLNGAV | 178 | 0.519 | 2.572 | 26.5 | PLNGAL | 677 | 0.463 | 0.590 | 0.09 |
| HLYAQA | | | | | HLYAQA | | | | |
| QSTPVQ | | | | | QTSPVQ | | | | |
| N | | | | | N | | | | |
| PLNGAV | 179 | 0.335 | 2.562 | 72.81 | ALNGAV | 678 | 0.399 | 0.589 | 1.59 |
| HLYAQA | | | | | HLYAQA | | | | |
| QTSPVQ | | | | | QTGWVQ | | | | |
| N | | | | | T | | | | |
| QLNGAV | 180 | 0.428 | 2.528 | 0.09 | PLNGAV | 679 | 0.193 | 0.586 | 0.47 |
| HLYAQA | | | | | HLYAQA | | | | |
| QVSQVQ | | | | | QTGWVK | | | | |
| N | | | | | K | | | | |
| PLNGAV | 181 | 0.625 | 2.523 | 19.96 | QLNGAV | 680 | 0.630 | 0.584 | 1450 |
| HLYAQA | | | | | HLYAQA | | | | |
| QTMQVQ | | | | | QTGWVQ | | | | |
| N | | | | | N | | | | |
| PLNGAV | 182 | 0.458 | 2.476 | 8.32 | PLNGDV | 681 | 0.630 | 0.580 | 0.09 |
| HLYAQA | | | | | HLYAQA | | | | |
| QTSKVQ | | | | | QTVAVQ | | | | |
| N | | | | | N | | | | |
| SLNGAV | 183 | 0.282 | 2.417 | 0.09 | PLNGGV | 682 | 0.501 | 0.580 | 0.47 |
| HLYAQA | | | | | HLYAQA | | | | |
| QVSPVQ | | | | | QPGWVQ | | | | |
| N | | | | | N | | | | |
| PLNGAV | 184 | 0.259 | 2.411 | 34.72 | PLNGAV | 683 | 0.897 | 0.579 | 7.66 |
| HLYAQA | | | | | HLYAQA | | | | |
| QVSQVQ | | | | | QAAAVQ | | | | |
| N | | | | | N | | | | |
| PLNGAV | 185 | 0.721 | 2.404 | 63.42 | PLNGAV | 684 | 0.618 | 0.579 | 19.72 |
| HLYAQA | | | | | HLYAQA | | | | |
| QVSPVQ | | | | | QSAVVQ | | | | |
| N | | | | | N | | | | |
| PLNGAV | 186 | 0.586 | 2.389 | 15.56 | TLNGAV | 685 | 0.646 | 0.578 | 0.09 |
| HLYAQA | | | | | HLYAQA | | | | |
| QTVQVQ | | | | | QRSSVQ | | | | |
| N | | | | | N | | | | |
| PLNGAV | 187 | 0.476 | 2.367 | 13.65 | PLNGAV | 686 | 0.214 | 0.578 | 0.14 |
| HLYAQA | | | | | HLYAQA | | | | |
| QVTAVQ | | | | | QPGWVQ | | | | |
| N | | | | | H | | | | |
| PLNGAV | 188 | 0.715 | 2.353 | 8.74 | PLDGAV | 687 | 0.214 | 0.577 | 0.09 |
| HLYAQA | | | | | HLYAQA | | | | |
| QRQPVQ | | | | | QTLSVQ | | | | |
| N | | | | | N | | | | |
| PLNGNV | 189 | 0.732 | 2.346 | 2.38 | PLNGAV | 688 | 0.310 | 0.575 | 0.19 |
| HLYAQA | | | | | HLYAQA | | | | |
| QTGGVQ | | | | | QTASDQ | | | | |
| N | | | | | N | | | | |
| PLDGAV | 190 | 0.697 | 2.339 | 7.06 | PLNGAV | 689 | 0.633 | 0.575 | 53.51 |
| HLYAQA | | | | | HLYAQA | | | | |
| QTAWVQ | | | | | QLSGVQ | | | | |
| N | | | | | N | | | | |
| PLNGAV | 191 | 0.785 | 2.331 | 9.91 | TLNGAV | 690 | 0.463 | 0.572 | 0.14 |
| HLYAQA | | | | | HLYAQA | | | | |
| QISGVQ | | | | | QTVAVQ | | | | |
| N | | | | | N | | | | |
| PLNGAV | 192 | 0.794 | 2.324 | 6.22 | PLNGAV | 691 | 0.293 | 0.572 | 0.09 |
| HLYAQA | | | | | HLYAQE | | | | |
| QVRPVQ | | | | | QVASVQ | | | | |
| N | | | | | N | | | | |
| PLNGAV | 193 | 0.722 | 2.299 | 20.47 | PVNGAV | 692 | 0.873 | 0.572 | 0.09 |
| HLYAQA | | | | | HLYAQA | | | | |
| QLGPVQ | | | | | QTGWAQ | | | | |
| N | | | | | N | | | | |
| PLNGAV | 194 | 0.404 | 2.290 | 4.91 | PLNGAV | 693 | 0.567 | 0.570 | 14.07 |
| HLYAQA | | | | | HLYAQA | | | | |
| QTNQVQ | | | | | QSSRVQ | | | | |
| N | | | | | N | | | | |
| PLNGAV | 195 | 0.291 | 2.240 | 14.39 | PLNGSV | 694 | 0.551 | 0.569 | 0.19 |
| HLYAQA | | | | | HLYAQA | | | | |
| QVQQVQ | | | | | QLGSVQ | | | | |
| N | | | | | N | | | | |
| PLNGAV | 196 | 0.520 | 2.238 | 9.21 | PLNGAV | 695 | 0.565 | 0.568 | 1.22 |
| HLYAQA | | | | | HLYAQA | | | | |
| QVANVQ | | | | | QSPPVQ | | | | |
| N | | | | | N | | | | |
| PLNGAV | 197 | 0.664 | 2.200 | 10.75 | PLNGAV | 696 | 0.605 | 0.567 | 0.14 |
| HLYAQA | | | | | HHYAQA | | | | |
| QAAPVQ | | | | | QTTSVQ | | | | |
| N | | | | | N | | | | |
| QLNGAV | 198 | 0.388 | 2.199 | 0.09 | PLNGAV | 697 | 0.832 | 0.567 | 0.09 |
| HLYAQA | | | | | HLYDQA | | | | |
| QVAQVQ | | | | | QSTPVQ | | | | |
| N | | | | | N | | | | |
| PLNGAV | 199 | 0.487 | 2.195 | 21.59 | QLNGAV | 698 | 0.529 | 0.567 | 0.37 |
| HLYAQA | | | | | HLYAQA | | | | |
| QRSTVQ | | | | | QTGWVR | | | | |
| N | | | | | N | | | | |
| PLNGAV | 200 | 0.334 | 2.190 | 29.72 | PLNGAV | 699 | 0.597 | 0.563 | 0.09 |
| HLYAQA | | | | | HLYAQA | | | | |
| QTMAVQ | | | | | QVQPVQ | | | | |
| N | | | | | T | | | | |
| PLNGAV | 201 | 0.838 | 2.182 | 7.1 | PLNGAV | 700 | 0.416 | 0.562 | 0.14 |
| HLYAQA | | | | | HLYAQA | | | | |
| QIQPVQ | | | | | QTSSVK | | | | |
| N | | | | | N | | | | |
| PLNGAV | 202 | 0.567 | 2.181 | 15.52 | PLNGAL | 701 | 0.301 | 0.560 | 0.09 |
| HLYAQA | | | | | HLYAQA | | | | |
| QIASVQ | | | | | QTASVQ | | | | |
| N | | | | | N | | | | |
| PLNGAV | 203 | 0.172 | 2.176 | 76.22 | PLNGAV | 702 | 0.731 | 0.558 | 0.09 |
| HLYAQA | | | | | HHYAQA | | | | |
| QTVSVQ | | | | | QSAPVQ | | | | |
| N | | | | | N | | | | |
| PLNGAV | 204 | 0.585 | 2.156 | 12.43 | PLNGSV | 703 | 0.169 | 0.556 | 0.47 |
| HLYAQA | | | | | HLYAQA | | | | |
| QRGSVQ | | | | | QTGWVH | | | | |
| N | | | | | N | | | | |
| PLNGAV | 205 | 0.376 | 2.143 | 12.85 | PLNGAV | 704 | 0.519 | 0.556 | 0.09 |
| HLYAQA | | | | | HLYAQA | | | | |
| QNSPVQ | | | | | QLSPLQ | | | | |
| N | | | | | N | | | | |
| PLNGAV | 206 | 0.363 | 2.131 | 24.3 | PLNGAV | 705 | 0.841 | 0.554 | 0.09 |
| HLYAQA | | | | | HLYAKA | | | | |
| QLQPVQ | | | | | QVTPVQ | | | | |
| N | | | | | N | | | | |
| PLNGAV | 207 | 0.550 | 2.125 | 11.68 | PLNGAV | 706 | 0.278 | 0.554 | 0.09 |
| HLYAQA | | | | | NLYAQA | | | | |
| QVTGVQ | | | | | QTQPVQ | | | | |
| N | | | | | N | | | | |
| PLNGAV | 208 | 0.810 | 2.121 | 11.26 | SLNGAV | 707 | 0.518 | 0.553 | 92.07 |
| HLYAQA | | | | | HLYAQA | | | | |
| QVMQVQ | | | | | QTGLVQ | | | | |
| N | | | | | N | | | | |
| PLNGAV | 209 | 0.697 | 2.104 | 14.86 | PLDGSV | 708 | 0.366 | 0.552 | 0.09 |
| HLYAQA | | | | | HLYAQA | | | | |
| QSMAVQ | | | | | QTGGVQ | | | | |
| N | | | | | N | | | | |
| PLNGAV | 210 | 0.611 | 2.104 | 4.49 | PLNGAV | 709 | 0.528 | 0.552 | 0.09 |
| HLYAQA | | | | | HLYDQA | | | | |
| QVGKVQ | | | | | QTLPVQ | | | | |
| N | | | | | N | | | | |
| ALNGAV | 211 | 0.652 | 2.101 | 0.09 | PLNGAV | 710 | 0.441 | 0.551 | 0.09 |
| HLYAQA | | | | | HLYAQA | | | | |
| QSAPVQ | | | | | QVSQVQ | | | | |
| N | | | | | K | | | | |
| PLNGAV | 212 | 0.498 | 2.101 | 3.97 | PLNGAV | 711 | 0.425 | 0.551 | 13.97 |
| HLYAQA | | | | | HLYAQA | | | | |
| QIQSVQ | | | | | QLVGVQ | | | | |
| N | | | | | N | | | | |
| PLNGAV | 213 | 0.537 | 2.097 | 17.15 | PLNGAV | 712 | 0.390 | 0.549 | 0.09 |
| HLYAQA | | | | | HLYAQE | | | | |
| QCSPVQ | | | | | QTSSVQ | | | | |
| N | | | | | N | | | | |
| PLNGAV | 214 | 0.821 | 2.079 | 3.97 | PLNGAV | 713 | 0.435 | 0.549 | 0.09 |
| HLYAQA | | | | | HIYAQA | | | | |
| QLQRVQ | | | | | QTVAVQ | | | | |
| N | | | | | N | | | | |
| PLNGAV | 215 | 0.330 | 2.063 | 0.09 | PLNGAV | 714 | 0.831 | 0.548 | 0.14 |
| HLYAQA | | | | | NLYAQA | | | | |
| QTAWVQ | | | | | QVTPVQ | | | | |
| H | | | | | N | | | | |
| PLDGAV | 216 | 0.650 | 2.062 | 0.09 | TLNGAV | 715 | 0.252 | 0.547 | 0.19 |
| HLYAQA | | | | | HLYAQA | | | | |
| QTGWDQ | | | | | QTMSVQ | | | | |
| N | | | | | N | | | | |
| PLNGAV | 217 | 0.411 | 2.062 | 0.89 | TLNGAV | 716 | 0.341 | 0.541 | 0.09 |
| HLYAQA | | | | | HLYAQA | | | | |
| QTPPVQ | | | | | QTLAVQ | | | | |
| N | | | | | N | | | | |
| PLNGAV | 218 | 0.635 | 2.054 | 1.68 | PINGAV | 717 | 0.341 | 0.540 | 0.09 |
| HLYAQA | | | | | HLYAQA | | | | |
| QVTKVQ | | | | | QVAQVQ | | | | |
| N | | | | | N | | | | |
| PLNGAV | 219 | 0.556 | 2.033 | 50.94 | PLNGQV | 718 | 0.885 | 0.540 | 12.57 |
| HLYAQA | | | | | HLYAQA | | | | |
| QSSPVQ | | | | | QTGWVQ | | | | |
| N | | | | | N | | | | |
| PLNGAV | 220 | 0.793 | 2.015 | 9.07 | QLNGAV | 719 | 0.892 | 0.540 | 39.63 |
| HLYAQA | | | | | HLYAQA | | | | |
| QAGPVQ | | | | | QTGGVQ | | | | |
| N | | | | | N | | | | |
| PLNGAV | 221 | 0.773 | 2.014 | 5.89 | PLNGAV | 720 | 0.542 | 0.539 | 0.09 |
| HLYAQA | | | | | HLYAQA | | | | |
| QLARVQ | | | | | QTLSVK | | | | |
| N | | | | | N | | | | |
| PLNGAV | 222 | 0.789 | 2.013 | 16.36 | PLNGTV | 721 | 0.331 | 0.538 | 0.37 |
| HLYAQA | | | | | HLYAQA | | | | |
| QTTTVQ | | | | | QTGWDQ | | | | |
| N | | | | | N | | | | |
| PLNGAV | 223 | 0.254 | 2.011 | 0.09 | PLNGAV | 722 | 0.476 | 0.537 | 0.14 |
| HLYAQA | | | | | HLYAQA | | | | |
| QTGGFQ | | | | | QTGWFQ | | | | |
| N | | | | | K | | | | |
| PLNGAV | 224 | 0.657 | 2.010 | 24.44 | ALNGAV | 723 | 0.447 | 0.536 | 0.09 |
| HLYAQA | | | | | HLYAQA | | | | |
| QTLQVQ | | | | | QTSKVQ | | | | |
| N | | | | | N | | | | |
| PLNGAV | 225 | 0.359 | 2.005 | 63.42 | ALNGTV | 724 | 0.712 | 0.535 | 49.16 |
| HLYAQA | | | | | HLYAQA | | | | |
| QTMSVQ | | | | | QTGWVQ | | | | |
| N | | | | | N | | | | |
| QLNGAV | 226 | 0.675 | 2.004 | 0.09 | PLNGAV | 725 | 0.256 | 0.534 | 0.14 |
| HLYAQA | | | | | PLYAQA | | | | |
| QLQPVQ | | | | | QTGWVQ | | | | |
| N | | | | | H | | | | |
| PLNGAV | 227 | 0.894 | 1.994 | 10.47 | PLNGAV | 726 | 0.355 | 0.533 | 0.23 |
| HLYAQA | | | | | HLYAQA | | | | |
| QVAKVQ | | | | | KTGWVQ | | | | |
| N | | | | | K | | | | |
| PLNGAV | 228 | 0.217 | 1.989 | 7.24 | PLNGAV | 727 | 0.492 | 0.531 | 1.12 |
| HLYAQA | | | | | HLYAQA | | | | |
| QRAAVQ | | | | | QTPAVQ | | | | |
| N | | | | | N | | | | |
| PLNGAV | 229 | 0.413 | 1.988 | 38.79 | PLNGAV | 728 | 0.538 | 0.531 | 0.14 |
| HLYAQA | | | | | HLYAQA | | | | |
| QTVGVQ | | | | | QTVGVK | | | | |
| N | | | | | N | | | | |
| PLNGAV | 230 | 0.381 | 1.972 | 15.84 | PLNGAV | 729 | 0.307 | 0.530 | 0.14 |
| HLYAQA | | | | | HLYAQE | | | | |
| QLNPVQ | | | | | QTGWFQ | | | | |
| N | | | | | N | | | | |
| PLNGAV | 231 | 0.379 | 1.971 | 26.17 | PLNGAV | 730 | 0.569 | 0.530 | 0.09 |
| HLYAQA | | | | | HLYAQA | | | | |
| QLSQVQ | | | | | QKGWDQ | | | | |
| N | | | | | N | | | | |
| PLNGTV | 232 | 0.421 | 1.967 | 23.37 | PLNGAV | 731 | 0.605 | 0.529 | 0.09 |
| HLYAQA | | | | | HLYDQA | | | | |
| QTGSVQ | | | | | QTSAVQ | | | | |
| N | | | | | N | | | | |
| PLNGAV | 233 | 0.543 | 1.960 | 12.95 | PLDGAV | 732 | 0.657 | 0.527 | 0.37 |
| HLYAQA | | | | | HLYAQA | | | | |
| QTKPVQ | | | | | QTGWVQ | | | | |
| N | | | | | K | | | | |
| PLNGAV | 234 | 0.603 | 1.957 | 7.71 | PLNGAV | 733 | 0.290 | 0.524 | 0.09 |
| HLYAQA | | | | | HLYAQA | | | | |
| QTNAVQ | | | | | QTAWVL | | | | |
| N | | | | | N | | | | |
| ALDGAV | 235 | 0.660 | 1.955 | 21.4 | PLNGSV | 734 | 0.172 | 0.524 | 0.28 |
| HLYAQA | | | | | HLYAQA | | | | |
| QTGWVQ | | | | | QTGWVL | | | | |
| N | | | | | N | | | | |
| PLNGAV | 236 | 0.303 | 1.955 | 22.06 | QLNGAV | 735 | 0.605 | 0.524 | 1.68 |
| HLYAQA | | | | | HLYAQA | | | | |
| QLATVQ | | | | | QTGWVQ | | | | |
| N | | | | | T | | | | |
| PLNGAV | 237 | 0.794 | 1.947 | 40.85 | PLNGAV | 736 | 0.699 | 0.524 | 0.98 |
| HLYAQA | | | | | HLYAQA | | | | |
| QVTPVQ | | | | | QVPPVQ | | | | |
| N | | | | | N | | | | |
| PLNGAV | 238 | 0.339 | 1.946 | 22.2 | PVNGAV | 737 | 0.734 | 0.522 | 0.09 |
| HLYAQA | | | | | HLYAQA | | | | |
| QVQAVQ | | | | | QVQSVQ | | | | |
| N | | | | | N | | | | |
| PLNGAV | 239 | 0.645 | 1.926 | 58.32 | QLNGAV | 738 | 0.473 | 0.522 | 0.09 |
| HLYAQA | | | | | HLYAQA | | | | |
| QTTSVQ | | | | | QTKSVQ | | | | |
| N | | | | | N | | | | |
| PLNGAV | 240 | 0.702 | 1.923 | 9.21 | PVNGAV | 739 | 0.825 | 0.522 | 0.23 |
| HLYAQA | | | | | HLYAQA | | | | |
| QCTPVQ | | | | | QTSSVQ | | | | |
| N | | | | | N | | | | |
| ALNGAV | 241 | 0.621 | 1.916 | 9.58 | PLNGAV | 740 | 0.797 | 0.520 | 11.45 |
| HLYAQA | | | | | HLYAQA | | | | |
| QTGRVQ | | | | | QATGVQ | | | | |
| N | | | | | N | | | | |
| PLNGSV | 242 | 0.173 | 1.908 | 0.09 | TLNGAV | 741 | 0.553 | 0.518 | 0.09 |
| HLYAQA | | | | | HLYAQA | | | | |
| QTGWVQ | | | | | QLSQVQ | | | | |
| D | | | | | N | | | | |
| PLNGAV | 243 | 0.555 | 1.903 | 30.47 | PLDGAV | 742 | 0.486 | 0.517 | 0.37 |
| HLYAQA | | | | | HLYAQA | | | | |
| QTAGVQ | | | | | QTVSVQ | | | | |
| N | | | | | N | | | | |
| PLNGAV | 244 | 0.431 | 1.902 | 29.82 | SLNGAV | 743 | 0.421 | 0.514 | 0.09 |
| HLYAQA | | | | | HLYAQA | | | | |
| QTSQVQ | | | | | QTKPVQ | | | | |
| N | | | | | N | | | | |
| PLNGAV | 245 | 0.443 | 1.895 | 6.4 | PLKGAV | 744 | 0.705 | 0.514 | 0.09 |
| HLYAQA | | | | | HLYAQA | | | | |
| QTMNVQ | | | | | QSAPVQ | | | | |
| N | | | | | N | | | | |
| PLNGAV | 246 | 0.323 | 1.885 | 34.16 | PLNGAV | 745 | 0.275 | 0.514 | 0.19 |
| HLYAQA | | | | | HLYAQA | | | | |
| QTSTVQ | | | | | KTGWVK | | | | |
| N | | | | | N | | | | |
| PLNGAV | 247 | 0.712 | 1.882 | 12.43 | PLNGSV | 746 | 0.680 | 0.513 | 0.09 |
| HLYAQA | | | | | HLYAQA | | | | |
| QVKPVQ | | | | | QTLGVQ | | | | |
| N | | | | | N | | | | |
| PLNGAV | 248 | 0.627 | 1.878 | 24.91 | PLKGAV | 747 | 0.626 | 0.512 | 0.09 |
| HLYAQA | | | | | HLYAQA | | | | |
| QASPVQ | | | | | QLAPVQ | | | | |
| N | | | | | N | | | | |
| PLNGAV | 249 | 0.447 | 1.873 | 23.27 | PLNGSV | 748 | 0.207 | 0.512 | 1.17 |
| HLYAQA | | | | | HLYAQA | | | | |
| QVAAVQ | | | | | QTGWVQ | | | | |
| N | | | | | K | | | | |
| PLNGAV | 250 | 0.692 | 1.869 | 8.74 | PLNGAV | 749 | 0.295 | 0.511 | 0.28 |
| HLYAQA | | | | | HLYAQA | | | | |
| QLKSVQ | | | | | QTGGVQ | | | | |
| N | | | | | I | | | | |
| PLNGAV | 251 | 0.834 | 1.855 | 8.09 | PLNGAV | 750 | 0.440 | 0.510 | 0.37 |
| HLYAQA | | | | | HLYAQP | | | | |
| QIAAVQ | | | | | QTGWVQ | | | | |
| N | | | | | T | | | | |
| PLNGAV | 252 | 0.115 | 1.850 | 26.78 | PLNGAV | 751 | 0.196 | 0.508 | 4.02 |
| HLYAQA | | | | | HLYAQA | | | | |
| QTAAVQ | | | | | QTGWVL | | | | |
| N | | | | | N | | | | |
| PLNGAV | 253 | 0.742 | 1.850 | 10.05 | PLNGAV | 752 | 0.611 | 0.507 | 0.14 |
| HLYAQA | | | | | HLYDQA | | | | |
| QTKAVQ | | | | | QLTPVQ | | | | |
| N | | | | | N | | | | |
| PLNGAV | 254 | 0.223 | 1.848 | 0.09 | QLNGAV | 753 | 0.558 | 0.507 | 0.28 |
| HLYAQA | | | | | HLYAQA | | | | |
| QTGSVQ | | | | | QTATVQ | | | | |
| S | | | | | N | | | | |
| PLNGAV | 255 | 0.518 | 1.845 | 7.66 | PLNGAV | 754 | 0.359 | 0.505 | 0.19 |
| HLYAQA | | | | | HLYAQA | | | | |
| QVSNVQ | | | | | QTAWGQ | | | | |
| N | | | | | N | | | | |
| PLNGAV | 25€ | 0.690 | 1.834 | 38.65 | PLNGAV | 755 | 0.273 | 0.505 | 6.78 |
| HLYAQA | | | | | HLYAQA | | | | |
| QTAPVQ | | | | | QTGWVH | | | | |
| N | | | | | N | | | | |
| PLNGAV | 257 | 0.740 | 1.833 | 28.13 | PLKGAV | 756 | 0.556 | 0.505 | 0.09 |
| HLYAQA | | | | | HLYAQA | | | | |
| QLMPVQ | | | | | QSSPVQ | | | | |
| N | | | | | N | | | | |
| PLNGAV | 258 | 0.798 | 1.824 | 8.88 | PLNGAV | 757 | 0.374 | 0.505 | 0.09 |
| HLYAQA | | | | | HLYAQA | | | | |
| QLHPVQ | | | | | QLGSVQ | | | | |
| N | | | | | T | | | | |
| PLNGAV | 259 | 0.793 | 1.823 | 5.09 | PLNGAV | 758 | 0.656 | 0.505 | 7.62 |
| HLYAQA | | | | | HLYAQA | | | | |
| QRAQVQ | | | | | QSANVQ | | | | |
| N | | | | | N | | | | |
| PLNGAV | 260 | 0.871 | 1.823 | 1.92 | PLNGGV | 759 | 0.866 | 0.503 | 23.27 |
| HLYAQA | | | | | HLYAQA | | | | |
| QLTNVQ | | | | | QTGSVQ | | | | |
| N | | | | | N | | | | |
| PLNGAV | 261 | 0.766 | 1.822 | 13.18 | PLNGAV | 760 | 0.433 | 0.503 | 0.09 |
| HLYAQA | | | | | HLYDQA | | | | |
| QRTTVQ | | | | | QRSSVQ | | | | |
| N | | | | | N | | | | |
| PLNGAV | 262 | 0.700 | 1.821 | 17.9 | PLNGAV | 761 | 0.164 | 0.502 | 0.19 |
| HLYAQA | | | | | HLYAQA | | | | |
| QTSVVQ | | | | | QTGGVQ | | | | |
| N | | | | | D | | | | |
| SLNGAV | 263 | 0.867 | 1.819 | 0.14 | PLNGAV | 762 | 0.271 | 0.502 | 0.09 |
| HLYAQA | | | | | HLYAQA | | | | |
| QTMSVQ | | | | | QTMAVK | | | | |
| N | | | | | N | | | | |
| ALNGAV | 264 | 0.672 | 1.818 | 0.09 | PLNGAV | 763 | 0.325 | 0.501 | 0.23 |
| HLYAQA | | | | | HLYAQA | | | | |
| QTLAVQ | | | | | QTASVK | | | | |
| N | | | | | N | | | | |
| PLNGAV | 265 | 0.736 | 1.816 | 12.2 | PLNGAV | 764 | 0.531 | 0.501 | 0.14 |
| HLYAQA | | | | | HLYAQA | | | | |
| QRMSVQ | | | | | QSSPVQ | | | | |
| N | | | | | K | | | | |
| ALNGAV | 266 | 0.342 | 1.813 | 0.14 | PLNGAV | 765 | 0.275 | 0.499 | 21.68 |
| HLYAQA | | | | | HLYAQA | | | | |
| QLTPVQ | | | | | QTGWVK | | | | |
| N | | | | | N | | | | |
| PLNGAV | 267 | 0.785 | 1.807 | 3.65 | ALNGAV | 766 | 0.735 | 0.499 | 25.05 |
| HLYAQA | | | | | HLYAQA | | | | |
| QVGNVQ | | | | | QTGGVQ | | | | |
| N | | | | | N | | | | |
| PLNGAV | 268 | 0.615 | 1.805 | 4.91 | PLNGAV | 767 | 0.676 | 0.498 | 0.09 |
| HLYAQA | | | | | HLYAQA | | | | |
| QLMQVQ | | | | | QTGTVQ | | | | |
| N | | | | | T | | | | |
| PLNGSV | 269 | 0.550 | 1.795 | 0.09 | PLNGAV | 768 | 0.476 | 0.495 | 7.43 |
| HLYAQA | | | | | HLYAQA | | | | |
| QTAQVQ | | | | | QTGWVQ | | | | |
| N | | | | | I | | | | |
| PLNGAV | 270 | 0.544 | 1.793 | 24.12 | PINGAV | 769 | 0.382 | 0.494 | 0.09 |
| HLYAQA | | | | | HLYAQA | | | | |
| QTATVQ | | | | | QTSLVQ | | | | |
| N | | | | | N | | | | |
| PLNGAV | 271 | 0.727 | 1.790 | 7.8 | PLNGAV | 770 | 0.502 | 0.492 | 0.09 |
| HLYAQA | | | | | HIYAQA | | | | |
| QVHPVQ | | | | | QVAQVQ | | | | |
| N | | | | | N | | | | |
| PLNGAV | 272 | 0.836 | 1.771 | 0.09 | PLNGAV | 771 | 0.279 | 0.491 | 0.19 |
| HLYAQA | | | | | HLYAQA | | | | |
| QVSPVQ | | | | | QTVWVK | | | | |
| T | | | | | N | | | | |
| PLNGAV | 273 | 0.638 | 1.766 | 9.11 | TLNGAV | 772 | 0.827 | 0.491 | 0.09 |
| HLYAQA | | | | | HLYAQA | | | | |
| QISSVQ | | | | | QLMPVQ | | | | |
| N | | | | | N | | | | |
| PLNGAV | 274 | 0.333 | 1.765 | 62.16 | PLNGAV | 773 | 0.672 | 0.490 | 0.09 |
| HLYAQA | | | | | HIYAQA | | | | |
| QVASVQ | | | | | QTAPVQ | | | | |
| N | | | | | N | | | | |
| PLNGAV | 275 | 0.347 | 1.757 | 0.09 | PLYGAV | 774 | 0.240 | 0.488 | 0.09 |
| HLYAQA | | | | | HLYAQA | | | | |
| QTRWDQ | | | | | QTASVQ | | | | |
| N | | | | | N | | | | |
| PLNGAV | 276 | 0.508 | 1.750 | 8.88 | TLNGAV | 775 | 0.643 | 0.488 | 0.09 |
| HLYAQA | | | | | HLYAQA | | | | |
| QTMTVQ | | | | | QLGPVQ | | | | |
| N | | | | | N | | | | |
| PLNGAV | 277 | 0.551 | 1.747 | 34.21 | PLDGAV | 776 | 0.637 | 0.487 | 0.42 |
| HLYAQA | | | | | HLYAQA | | | | |
| QRSSVQ | | | | | QTGWVK | | | | |
| N | | | | | N | | | | |
| PLNGGV | 278 | 0.547 | 1.743 | 0.09 | PLKGAV | 777 | 0.669 | 0.487 | 0.14 |
| HLYAQA | | | | | HLYAQA | | | | |
| QTGWVR | | | | | QVSPVQ | | | | |
| N | | | | | N | | | | |
| PLNGAV | 279 | 0.281 | 1.741 | 213 | PLNGAV | 778 | 0.563 | 0.486 | 9.95 |
| HLYAQA | | | | | HLYAQA | | | | |
| QTAWVQ | | | | | QSQQVQ | | | | |
| N | | | | | N | | | | |
| PLNGSV | 280 | 0.130 | 1.734 | 0.28 | PLNGAV | 779 | 0.497 | 0.484 | 0.09 |
| HLYAQA | | | | | DLYAQA | | | | |
| QPGWVQ | | | | | QLSPVQ | | | | |
| Z | | | | | N | | | | |
| ALNGAV | 281 | 0.238 | 1.724 | 0.09 | PLNGAV | 780 | 0.506 | 0.484 | 0.09 |
| HLYAQA | | | | | HLYAQA | | | | |
| QTGWAQ | | | | | QVGSVQ | | | | |
| N | | | | | T | | | | |
| PLNGAV | 282 | 0.791 | 1.724 | 8.93 | PLNGTV | 781 | 0.405 | 0.483 | 0.14 |
| HLYAQA | | | | | HLYAQA | | | | |
| QRTGVQ | | | | | QTGWAQ | | | | |
| N | | | | | N | | | | |
| ALNGAV | 283 | 0.370 | 1.720 | 0.09 | ALNGAV | 782 | 0.283 | 0.482 | 0.09 |
| HLYAQA | | | | | HLYAQA | | | | |
| QTGWVQ | | | | | QTGLVQ | | | | |
| S | | | | | T | | | | |
| PLNGAV | 284 | 0.561 | 1.701 | 22.34 | PLNGAV | 783 | 0.562 | 0.482 | 35.42 |
| HLYAQA | | | | | HLYAQA | | | | |
| QVATVQ | | | | | QSSMVQ | | | | |
| N | | | | | N | | | | |
| PLNGAV | 285 | 0.480 | 1.700 | 16.68 | PLNGAV | 784 | 0.484 | 0.481 | 0.09 |
| HLYAQA | | | | | HLYAKA | | | | |
| QVTSVQ | | | | | QTAGVQ | | | | |
| N | | | | | N | | | | |
| PLNGAV | 286 | 0.313 | 1.699 | 74.78 | PLNGAV | 785 | 0.516 | 0.480 | 0.09 |
| HLYAQA | | | | | HLYAHA | | | | |
| QVSSVQ | | | | | QTGWVQ | | | | |
| N | | | | | K | | | | |
| PLNGSV | 287 | 0.353 | 1.683 | 0.14 | PLNGAV | 786 | 0.502 | 0.479 | 0.23 |
| HLYAQA | | | | | HLYAQA | | | | |
| QTSSVQ | | | | | QTSPVQ | | | | |
| N | | | | | K | | | | |
| PLNGAV | 288 | 0.348 | 1.682 | 18.83 | PLNGAV | 787 | 0.841 | 0.477 | 7.66 |
| HLYAQA | | | | | HLYAQA | | | | |
| QTNSVQ | | | | | QAVGVQ | | | | |
| N | | | | | N | | | | |
| PLNGAV | 289 | 0.815 | 1.681 | 6.03 | PLNGAV | 788 | 0.438 | 0.477 | 0.09 |
| HLYAQA | | | | | PLYAQA | | | | |
| QVKAVQ | | | | | QTGWGQ | | | | |
| N | | | | | N | | | | |
| PLNGAV | 290 | 0.689 | 1.666 | 19.02 | PLNGTV | 789 | 0.495 | 0.477 | 0.09 |
| HLYAQA | | | | | HLYAQA | | | | |
| QSGPVQ | | | | | QLGSVQ | | | | |
| N | | | | | N | | | | |
| PLNGAV | 291 | 0.660 | 1.663 | 25.61 | QLNGAV | 790 | 0.645 | 0.476 | 0.75 |
| HLYAQA | | | | | HLYAQA | | | | |
| QTGPVQ | | | | | QTGWVQ | | | | |
| N | | | | | H | | | | |
| PLNGAV | 292 | 0.537 | 1.660 | 22.9 | PLNGSV | 791 | 0.175 | 0.475 | 1.4 |
| HLYAQA | | | | | HLYAQA | | | | |
| QTAMVQ | | | | | QTGWVK | | | | |
| N | | | | | N | | | | |
| PLNGAV | 293 | 0.358 | 1.628 | 32.11 | PLNGAV | 792 | 0.812 | 0.475 | 0.09 |
| HLYAQA | | | | | HLYDQA | | | | |
| QTQPVQ | | | | | QVAPVQ | | | | |
| N | | | | | N | | | | |
| PLNGSV | 294 | 0.533 | 1.625 | 32.76 | PLNGAV | 793 | 0.460 | 0.473 | 0.19 |
| HLYAQA | | | | | NLYAQA | | | | |
| QTGSVQ | | | | | QVSSVQ | | | | |
| N | | | | | N | | | | |
| PLNGAV | 295 | 0.519 | 1.625 | 14.44 | PLNGAV | 794 | 0.646 | 0.472 | 0.09 |
| HLYAQA | | | | | HLYAQA | | | | |
| QTQQVQ | | | | | QTLPVQ | | | | |
| N | | | | | K | | | | |
| PLNGAV | 296 | 0.824 | 1.620 | 9.86 | PLNGSV | 795 | 0.337 | 0.469 | 0.93 |
| HLYAQA | | | | | HLYAQA | | | | |
| QVSRVQ | | | | | QTASVQ | | | | |
| N | | | | | N | | | | |
| QLNGAV | 297 | 0.436 | 1.617 | 0.09 | ALNGAV | 796 | 0.294 | 0.468 | 0.37 |
| HLYAQA | | | | | HLYAQA | | | | |
| QVLPVQ | | | | | QTGWGQ | | | | |
| N | | | | | N | | | | |
| PLNGAV | 298 | 0.266 | 1.610 | 0.14 | PLNGAV | 797 | 0.286 | 0.468 | 7.48 |
| HLYAQA | | | | | HLYAQA | | | | |
| QTGWVQ | | | | | QTGWGQ | | | | |
| P | | | | | N | | | | |
| QLNGAV | 299 | 0.776 | 1.609 | 0.09 | PLNGAA | 798 | 0.451 | 0.467 | 0.09 |
| HLYAQA | | | | | HLYAQA | | | | |
| QLGSVQ | | | | | QTSPVQ | | | | |
| N | | | | | N | | | | |
| PLNGAV | 300 | 0.360 | 1.604 | 40.29 | PLNGAV | 799 | 0.646 | 0.465 | 0.14 |
| HLYAQA | | | | | HLYAQA | | | | |
| QVSAVQ | | | | | QTGRVQ | | | | |
| N | | | | | H | | | | |
| PLNGAV | 301 | 0.593 | 1.604 | 7.8 | PINGAV | 800 | 0.768 | 0.464 | 0.09 |
| HLYAQA | | | | | HLYAQA | | | | |
| QVLSVQ | | | | | QTGWVQ | | | | |
| N | | | | | S | | | | |
| PLNGAV | 302 | 0.861 | 1.601 | 5.05 | PLNGAV | 801 | 0.454 | 0.464 | 2.24 |
| HLYAQA | | | | | HLYAQA | | | | |
| QTQHVQ | | | | | QTP SV | | | | |
| N | | | | | QN | | | | |
| PLNGAV | 303 | 0.113 | 1.590 | 52.72 | PLNGAV | 802 | 0.450 | 0.464 | 0.09 |
| HLYAQA | | | | | HLYDQA | | | | |
| QLASVQ | | | | | QTTAVQ | | | | |
| N | | | | | N | | | | |
| PLNGAV | 304 | 0.649 | 1.581 | 8.83 | PLNGNV | 803 | 0.415 | 0.462 | 0.09 |
| HLYAQA | | | | | HLYAQA | | | | |
| QQAPVQ | | | | | QTGWVQ | | | | |
| N | | | | | K | | | | |
| PLNGAV | 305 | 0.577 | 1.574 | 3.08 | PLNGAV | 804 | 0.167 | 0.462 | 2.62 |
| HLYAQA | | | | | HLYAQA | | | | |
| QNAQVQ | | | | | QTGWVE | | | | |
| N | | | | | N | | | | |
| PLNGAV | 306 | 0.760 | 1.567 | 12.81 | PLNGAV | 805 | 0.371 | 0.461 | 0.19 |
| HLYAQA | | | | | NLYAQA | | | | |
| QATPVQ | | | | | QTTPVQ | | | | |
| N | | | | | N | | | | |
| PLNGAV | 307 | 0.544 | 1.567 | 28.13 | PLNGAV | 806 | 0.489 | 0.461 | 0.09 |
| HLYAQA | | | | | NLYAQA | | | | |
| QVQPVQ | | | | | QLSQVQ | | | | |
| N | | | | | N | | | | |
| PLNGAV | 308 | 0.537 | 1.563 | 33.13 | SLNGAV | 807 | 0.263 | 0.460 | 0.14 |
| HLYAQA | | | | | HLYAQA | | | | |
| QTTAVQ | | | | | QVMSVQ | | | | |
| N | | | | | N | | | | |
| PLNGAV | 309 | 0.455 | 1.561 | 3.46 | QLNGAV | 808 | 0.622 | 0.458 | 0.33 |
| HLYAQA | | | | | HLYAQA | | | | |
| QTGWVR | | | | | QTGWVQ | | | | |
| N | | | | | S | | | | |
| PLNGAV | 310 | 0.357 | 1.561 | 21.83 | PINGAV | 809 | 0.534 | 0.457 | 0.19 |
| HLYAQA | | | | | HLYAQA | | | | |
| QLAAVQ | | | | | QTGWFQ | | | | |
| N | | | | | N | | | | |
| PLNGAV | 311 | 0.418 | 1.555 | 0.09 | PLNGDV | 810 | 0.419 | 0.456 | 0.14 |
| HLYAQA | | | | | HLYAQA | | | | |
| QTSPDQ | | | | | QVSSVQ | | | | |
| N | | | | | N | | | | |
| PLNGAV | 312 | 0.393 | 1.553 | 16.36 | PLNGGV | 811 | 0.482 | 0.456 | 0.28 |
| HLYAQA | | | | | HLYAQA | | | | |
| QRSGVQ | | | | | QTGWVQ | | | | |
| N | | | | | S | | | | |
| PLNGAV | 313 | 0.218 | 1.551 | 0.65 | PLNGAV | 812 | 0.559 | 0.456 | 0.09 |
| HLYAQA | | | | | NLYAQA | | | | |
| QTGGVQ | | | | | QRSTVQ | | | | |
| T | | | | | N | | | | |
| PLNGAV | 314 | 0.274 | 1.546 | 0.93 | ALNGAV | 813 | 0.298 | 0.455 | 0.14 |
| HLYAQA | | | | | HLYAQA | | | | |
| QTGWVP | | | | | QTGWVQ | | | | |
| N | | | | | Y | | | | |
| PLDSAV | 315 | 0.833 | 1.539 | 10.89 | PLNGAV | 814 | 0.170 | 0.455 | 0.09 |
| HLYAQA | | | | | HHYAQA | | | | |
| QTGWVQ | | | | | QTAAVQ | | | | |
| N | | | | | N | | | | |
| PLNGAV | 316 | 0.496 | 1.537 | 0.09 | PLNGAV | 815 | 0.346 | 0.454 | 0.09 |
| HLYAQA | | | | | HRYAQA | | | | |
| QTTPVQ | | | | | QTGWVQ | | | | |
| T | | | | | H | | | | |
| PLNGAV | 317 | 0.540 | 1.535 | 7.94 | QLNGAV | 816 | 0.627 | 0.453 | 0.37 |
| HLYAQA | | | | | HLYAQA | | | | |
| QLMAVQ | | | | | QTGWVH | | | | |
| N | | | | | N | | | | |
| PLNGAV | 318 | 0.596 | 1.532 | 36.69 | PLNGAV | 817 | 0.861 | 0.452 | 56.6 |
| HLYAQA | | | | | HLYAQA | | | | |
| QTMPVQ | | | | | QRGWVQ | | | | |
| N | | | | | N | | | | |
| PLNGAV | 319 | 0.391 | 1.529 | 0.09 | PLNGAV | 818 | 0.233 | 0.450 | 0.14 |
| HHYAQA | | | | | HLYAQA | | | | |
| QTSPVQ | | | | | QTGSVE | | | | |
| N | | | | | N | | | | |
| PLNGAV | 320 | 0.756 | 1.527 | 7.8 | ALNGAV | 819 | 0.249 | 0.450 | 0.37 |
| HLYAQA | | | | | HLYAQA | | | | |
| QLANVQ | | | | | QTGWVH | | | | |
| N | | | | | N | | | | |
| PLNGAV | 321 | 0.606 | 1.520 | 11.68 | PLNGAV | 820 | 0.494 | 0.449 | 0.09 |
| HLYAQA | | | | | NLYAQA | | | | |
| QVSTVQ | | | | | QTLAVQ | | | | |
| N | | | | | N | | | | |
| PLNGAV | 322 | 0.602 | 1.518 | 12.9 | PLNGAV | 821 | 0.772 | 0.448 | 91.93 |
| HLYAQA | | | | | HLYAQA | | | | |
| QSAQVQ | | | | | QSSLVQ | | | | |
| N | | | | | N | | | | |
| PLNGAV | 323 | 0.838 | 1.508 | 8.13 | ALNGAV | 822 | 0.203 | 0.448 | 0.37 |
| HLYAQA | | | | | HLYAQA | | | | |
| QNTPVQ | | | | | QTGWLQ | | | | |
| N | | | | | N | | | | |
| PLNGAV | 324 | 0.381 | 1.495 | 0.09 | TLNGAV | 823 | 0.736 | 0.448 | 0.09 |
| HLYAQA | | | | | HLYAQA | | | | |
| QVSSVQ | | | | | QTLQVQ | | | | |
| T | | | | | N | | | | |
| PLNGAV | 325 | 0.284 | 1.492 | 0.09 | PLNGAV | 824 | 0.439 | 0.448 | 0.09 |
| HLYAQA | | | | | HLYDQA | | | | |
| QTVSVK | | | | | QVQSVQ | | | | |
| N | | | | | N | | | | |
| PLNGAV | 326 | 0.710 | 1.488 | 9.21 | SLNGAV | 825 | 0.442 | 0.447 | 0.09 |
| HLYAQP | | | | | HLYAQA | | | | |
| QTGLVQ | | | | | QVTGVQ | | | | |
| N | | | | | N | | | | |
| PLNGAV | 327 | 0.287 | 1.487 | 621.2 | QLNGAV | 826 | 0.849 | 0.444 | 0.09 |
| HLYAQA | | | | | HLYAQA | | | | |
| QTGSVQ | | | | | QSAVVQ | | | | |
| N | | | | | N | | | | |
| SLNGAV | 328 | 0.437 | 1.485 | 0.09 | PLNGVV | 827 | 0.410 | 0.444 | 0.09 |
| HLYAQA | | | | | HLYAQA | | | | |
| QTLPVQ | | | | | QTGWVQ | | | | |
| N | | | | | T | | | | |
| PLNGAV | 329 | 0.440 | 1.478 | 0.09 | PLNGAV | 828 | 0.373 | 0.444 | 0.61 |
| HLYAQA | | | | | HLYAQA | | | | |
| QTGGAQ | | | | | QTAWVK | | | | |
| N | | | | | N | | | | |
| SLNGAV | 330 | 0.618 | 1.477 | 0.09 | TLNGAV | 829 | 0.557 | 0.443 | 0.14 |
| HLYAQA | | | | | HLYAQA | | | | |
| QTAQVQ | | | | | QTLPVQ | | | | |
| N | | | | | N | | | | |
| PLNGAV | 331 | 0.684 | 1.466 | 14.21 | ALNGAV | 830 | 0.683 | 0.442 | 0.09 |
| HLYAQA | | | | | HLYAQA | | | | |
| QTAVVQ | | | | | QTTGVQ | | | | |
| N | | | | | N | | | | |
| PLNGAV | 332 | 0.533 | 1.456 | 17.71 | PLNGAV | 831 | 0.466 | 0.442 | 0.09 |
| HLYAQA | | | | | HLYAKA | | | | |
| QRLGVQ | | | | | QTMAVQ | | | | |
| N | | | | | N | | | | |
| PLNGSV | 333 | 0.207 | 1.453 | 0.37 | PLNGAV | 832 | 0.636 | 0.442 | 10.61 |
| HLYAQA | | | | | HLYAQA | | | | |
| QTGWVQ | | | | | QTYSVQ | | | | |
| H | | | | | N | | | | |
| PLNGAV | 334 | 0.489 | 1.452 | 21.26 | PLNGCV | 833 | 0.285 | 0.441 | 0.75 |
| HLYAQA | | | | | HLYAQA | | | | |
| QRTLVQ | | | | | QTGWVQ | | | | |
| N | | | | | N | | | | |
| SLNGAV | 335 | 0.559 | 1.451 | 0.09 | PLNGAV | 834 | 0.491 | 0.440 | 0.09 |
| HLYAQA | | | | | HLYAQA | | | | |
| QTMAVQ | | | | | QLAAVQ | | | | |
| Z | | | | | T | | | | |
| PLNGAV | 336 | 0.707 | 1.447 | 10.23 | PLNGAV | 835 | 0.734 | 0.440 | 0.14 |
| HLYAQA | | | | | HLYAQA | | | | |
| QTQMVQ | | | | | QTALVK | | | | |
| N | | | | | N | | | | |
| PLNGAV | 337 | 0.663 | 1.442 | 10.52 | PLNGAV | 836 | 0.392 | 0.439 | 0.14 |
| HLYAQA | | | | | HLYAKA | | | | |
| QITPVQ | | | | | QTVAVQ | | | | |
| N | | | | | N | | | | |
| PLNGAV | 338 | 0.199 | 1.434 | 0.09 | PLNGAV | 837 | 0.225 | 0.439 | 0.37 |
| HLYAQA | | | | | HLYAHA | | | | |
| QTVWVQ | | | | | QTGWVQ | | | | |
| K | | | | | T | | | | |
| PLNGAV | 339 | 0.584 | 1.423 | 13.97 | PLNGGV | 838 | 0.649 | 0.439 | 0.09 |
| HLYAQA | | | | | HLYAQA | | | | |
| QRSAVQ | | | | | QRGSVQ | | | | |
| N | | | | | N | | | | |
| PLNGAV | 340 | 0.332 | 1.420 | 99.87 | PLNGAF | 839 | 0.462 | 0.436 | 0.14 |
| HLYAQA | | | | | HLYAQA | | | | |
| QTASVQ | | | | | QTGGVQ | | | | |
| N | | | | | N | | | | |
| PLNGAV | 341 | 0.536 | 1.417 | 11.22 | PLNGAV | 840 | 0.496 | 0.436 | 0.09 |
| HLYAQA | | | | | HLYALA | | | | |
| QTMGVQ | | | | | QTSPVQ | | | | |
| N | | | | | N | | | | |
| PLNGAV | 342 | 0.256 | 1.414 | 0.28 | PLNGAV | 841 | 0.225 | 0.435 | 2.9 |
| HLYAQA | | | | | HLYAQA | | | | |
| QTGGVQ | | | | | QTGWVQ | | | | |
| H | | | | | Y | | | | |
| PLNGAV | 343 | 0.415 | 1.412 | 36.36 | PLNGAV | 842 | 0.538 | 0.432 | 0.19 |
| HLYAQA | | | | | HLYAQA | | | | |
| QVQSVQ | | | | | QTLPVQ | | | | |
| N | | | | | T | | | | |
| PLNGAV | 344 | 0.324 | 1.411 | 500.1 | PLNGAV | 843 | 0.499 | 0.432 | 0.23 |
| HLYAQA | | | | | HLYSQA | | | | |
| QTGGVQ | | | | | QTGWFQ | | | | |
| N | | | | | N | | | | |
| SLNGAV | 345 | 0.471 | 1.411 | 0.09 | PLNGAV | 844 | 0.696 | 0.432 | 0.33 |
| HLYAQA | | | | | HLYAQA | | | | |
| QTAPVQ | | | | | QTGLVQ | | | | |
| N | | | | | H | | | | |
| PLNGAV | 346 | 0.680 | 1.410 | 16.68 | PLNGAV | 845 | 0.628 | 0.432 | 0.98 |
| HLYAQA | | | | | HLYAQA | | | | |
| QISPVQ | | | | | QTP TV | | | | |
| N | | | | | QN | | | | |
| PLNGAV | 347 | 0.345 | 1.409 | 0.09 | PLYGAV | 846 | 0.274 | 0.429 | 0.28 |
| HLYAQP | | | | | HLYAQA | | | | |
| QTGWVK | | | | | QTAWVQ | | | | |
| N | | | | | N | | | | |
| PLNGAV | 348 | 0.242 | 1.409 | 0.14 | PLNGAV | 847 | 0.275 | 0.428 | 0.23 |
| HLYAQA | | | | | HLYAKA | | | | |
| QTGSAQ | | | | | QTASVQ | | | | |
| N | | | | | N | | | | |
| PLNGAV | 349 | 0.474 | 1.393 | 2.62 | PLNGAV | 848 | 0.285 | 0.428 | 0.19 |
| HLYAQA | | | | | HLYAQA | | | | |
| QTGWAQ | | | | | QTASVQ | | | | |
| N | | | | | K | | | | |
| PLNGAV | 350 | 0.383 | 1.392 | 0.09 | PLDGSV | 849 | 0.641 | 0.424 | 0.09 |
| HLYAQA | | | | | HLYAQA | | | | |
| QTMSVQ | | | | | QTAWVQ | | | | |
| T | | | | | N | | | | |
| PLNGSV | 351 | 0.649 | 1.391 | 12.29 | PLNGAD | 850 | 0.540 | 0.423 | 0.09 |
| HLYAQA | | | | | HLYAQA | | | | |
| QTAWVQ | | | | | QTSSVQ | | | | |
| N | | | | | N | | | | |
| PLNGAV | 352 | 0.410 | 1.387 | 8.13 | PLNGAV | 851 | 0.594 | 0.422 | 0.09 |
| HLYAQA | | | | | HLYAQA | | | | |
| QVGGVQ | | | | | QTSPVQ | | | | |
| N | | | | | I | | | | |
| PLNGAV | 353 | 0.496 | 1.387 | 0.09 | PLNGAV | 852 | 0.661 | 0.420 | 40 |
| HLYAKA | | | | | HLYAQA | | | | |
| QTSPVQ | | | | | QIGWVQ | | | | |
| N | | | | | N | | | | |
| PLNGAV | 354 | 0.768 | 1.381 | 0.09 | PLNGAV | 853 | 0.209 | 0.419 | 0.09 |
| HLYAQA | | | | | HLYAKA | | | | |
| QLAPVQ | | | | | QTSTVQ | | | | |
| T | | | | | N | | | | |
| PLDGAV | 355 | 0.901 | 1.375 | 0.09 | PLNGAV | 854 | 0.237 | 0.419 | 0.19 |
| HLYAQA | | | | | HRYAQA | | | | |
| QTALVQ | | | | | QTGWVQ | | | | |
| N | | | | | T | | | | |
| PLNGAV | 356 | 0.594 | 1.368 | 58.98 | PLNGAV | 855 | 0.268 | 0.419 | 0.14 |
| HLYAQA | | | | | HLYDQA | | | | |
| QTALVQ | | | | | QTSTVQ | | | | |
| N | | | | | N | | | | |
| PLNGAV | 357 | 0.470 | 1.366 | 19.63 | QLNGAV | 856 | 0.568 | 0.418 | 69.31 |
| HLYAQA | | | | | HLYAQA | | | | |
| QLAGVQ | | | | | QTGLVQ | | | | |
| N | | | | | N | | | | |
| PLNGAV | 358 | 0.613 | 1.365 | 9.53 | PLNGAV | 857 | 0.864 | 0.417 | 0.09 |
| HLYAQA | | | | | NLYAQA | | | | |
| QRTAVQ | | | | | QLGPVQ | | | | |
| N | | | | | N | | | | |
| PLNGAV | 359 | 0.540 | 1.363 | 22.15 | PLNGAV | 858 | 0.400 | 0.417 | 0.23 |
| HLYAQA | | | | | HLYAQA | | | | |
| QRSPVQ | | | | | QTGWDK | | | | |
| N | | | | | N | | | | |
| PLNGAV | 360 | 0.513 | 1.360 | 39.82 | PLNGAL | 859 | 0.432 | 0.416 | 0.14 |
| HLYAQA | | | | | HLYAQA | | | | |
| QTLAVQ | | | | | QTGWVK | | | | |
| N | | | | | N | | | | |
| PLNGAV | 361 | 0.322 | 1.351 | 5.42 | PLNGNV | 860 | 0.698 | 0.414 | 0.09 |
| HLYAQA | | | | | HLYAHA | | | | |
| QLAHVQ | | | | | QTGWVQ | | | | |
| N | | | | | N | | | | |
| PLNGAV | 362 | 0.352 | 1.348 | 36.69 | PLNGAV | 861 | 0.861 | 0.414 | 8.23 |
| HLYAQA | | | | | HLYAQA | | | | |
| QTSLVQ | | | | | QKSSVQ | | | | |
| N | | | | | N | | | | |
| PLNGAV | 363 | 0.548 | 1.347 | 30.75 | PLNGAV | 862 | 0.691 | 0.412 | 30.7 |
| HLYAQA | | | | | HLYAQA | | | | |
| QRLSVQ | | | | | QTGYVQ | | | | |
| N | | | | | N | | | | |
| PLNGAV | 364 | 0.406 | 1.344 | 0.09 | PVNGAV | 863 | 0.456 | 0.412 | 0.09 |
| HLYDQA | | | | | HLYAQA | | | | |
| QLSPVQ | | | | | QVGSVQ | | | | |
| N | | | | | N | | | | |
| PLNGAV | 365 | 0.424 | 1.339 | 13.97 | PLNGGV | 864 | 0.536 | 0.411 | 1.17 |
| HLYAQA | | | | | HLYAQA | | | | |
| QLMGVQ | | | | | QTGWVK | | | | |
| N | | | | | N | | | | |
| PLNGSV | 366 | 0.147 | 1.336 | 1.03 | PLNGAV | 865 | 0.627 | 0.410 | 0.09 |
| HLYAQA | | | | | HLYAQA | | | | |
| QTGWVQ | | | | | QRGWVQ | | | | |
| T | | | | | T | | | | |
| PLNGAV | 367 | 0.588 | 1.335 | 11.26 | PLNGAV | 866 | 0.729 | 0.410 | 0.09 |
| HLYAQA | | | | | NLYAQA | | | | |
| QSMQVQ | | | | | QRSLVQ | | | | |
| N | | | | | N | | | | |
| PLNGGV | 368 | 0.483 | 1.334 | 0.14 | PINGAV | 867 | 0.358 | 0.410 | 0.51 |
| HLYAQA | | | | | HLYAQA | | | | |
| QTGWFQ | | | | | QTGWDQ | | | | |
| N | | | | | N | | | | |
| PLNGAV | 369 | 0.834 | 1.326 | 14.02 | PLNGNV | 868 | 0.536 | 0.410 | 0.23 |
| HLYAQA | | | | | HLYAQA | | | | |
| QTQTVQ | | | | | QTGWVQ | | | | |
| N | | | | | T | | | | |
| PLDGAV | 370 | 0.860 | 1.323 | 9.44 | PLNGAV | 869 | 0.265 | 0.409 | 0.23 |
| HLYSQA | | | | | HLYAKA | | | | |
| QTGWVQ | | | | | QTGWFQ | | | | |
| N | | | | | N | | | | |
| PLNGAV | 371 | 0.325 | 1.319 | 0.14 | PLNGAV | 870 | 0.421 | 0.408 | 0.09 |
| HLYAQA | | | | | HLYAQE | | | | |
| QTGWEQ | | | | | QTSTVQ | | | | |
| N | | | | | N | | | | |
| PLNGAV | 372 | 0.487 | 1.317 | 29.44 | PLNGAV | 871 | 0.281 | 0.406 | 0.14 |
| HLYAQA | | | | | HLYAQA | | | | |
| QVGSVQ | | | | | QTGGVE | | | | |
| N | | | | | N | | | | |
| PLNGAV | 373 | 0.339 | 1.317 | 24.86 | PLNGAV | 872 | 0.831 | 0.406 | 0.09 |
| HLYAQA | | | | | HLYSQA | | | | |
| QVSGVQ | | | | | QTGWVQ | | | | |
| N | | | | | I | | | | |
| PLNGAV | 374 | 0.767 | 1.312 | 13.04 | SLNGAV | 873 | 0.480 | 0.406 | 0.09 |
| HLYAQA | | | | | HLYAQA | | | | |
| QVMAVQ | | | | | QTGLVQ | | | | |
| N | | | | | K | | | | |
| PLNGAV | 375 | 0.814 | 1.309 | 23.37 | PLNGAV | 874 | 0.605 | 0.403 | 0.09 |
| HLYAQA | | | | | HLYAQA | | | | |
| QIGGVQ | | | | | QTGCVR | | | | |
| N | | | | | N | | | | |
| PLNGAV | 376 | 0.841 | 1.301 | 5.47 | PLNGAD | 875 | 0.758 | 0.402 | 0.09 |
| HLYAQA | | | | | HLYAQA | | | | |
| QIAGVQ | | | | | QVTPVQ | | | | |
| N | | | | | N | | | | |
| PLNGAV | 377 | 0.200 | 1.297 | 0.09 | PLNGAV | 876 | 0.129 | 0.400 | 0.28 |
| HLYAKA | | | | | HLYAPA | | | | |
| QTVSVQ | | | | | QTGWVQ | | | | |
| N | | | | | T | | | | |
| PLNGAV | 378 | 0.838 | 1.297 | 4.3 | PLNGDV | 877 | 0.900 | 0.400 | 0.09 |
| HLYSQA | | | | | HLYAQA | | | | |
| QTGRVQ | | | | | QTSAVQ | | | | |
| N | | | | | N | | | | |
| PLNGAV | 379 | 0.826 | 1.296 | 7.34 | TLNGAV | 878 | 0.635 | 0.399 | 0.09 |
| HLYAQA | | | | | HLYAQA | | | | |
| QLSHVQ | | | | | QSSLVQ | | | | |
| N | | | | | N | | | | |
| PLNGAV | 380 | 0.865 | 1.288 | 4.77 | SLNGAV | 879 | 0.645 | 0.399 | 0.14 |
| HLYAQA | | | | | HLYAQA | | | | |
| QVQTVQ | | | | | QRSVVQ | | | | |
| N | | | | | N | | | | |
| SLDGAV | 381 | 0.875 | 1.287 | 61.08 | SLNGAV | 880 | 0.817 | 0.397 | 0.33 |
| HLYAQA | | | | | HLYAQA | | | | |
| QTGWVQ | | | | | QTGWVQ | | | | |
| N | | | | | S | | | | |
| PLNGAV | 382 | 0.326 | 1.287 | 0.09 | PVNGAV | 881 | 0.800 | 0.397 | 0.14 |
| HLYAQP | | | | | HLYAQA | | | | |
| QTGWDQ | | | | | QTGWVQ | | | | |
| N | | | | | S | | | | |
| PLNGAV | 383 | 0.564 | 1.284 | 11.36 | PLNGAV | 882 | 0.619 | 0.396 | 0.09 |
| HLYAQA | | | | | HLYAQA | | | | |
| QRNSVQ | | | | | QLSPVQ | | | | |
| N | | | | | S | | | | |
| PLNGAV | 384 | 0.533 | 1.283 | 57.16 | PLNGAV | 883 | 0.285 | 0.395 | 0.09 |
| HLYAQA | | | | | HLYAQP | | | | |
| QTLPVQ | | | | | QTGWLQ | | | | |
| N | | | | | N | | | | |
| PLNGAV | 385 | 0.569 | 1.282 | 5.09 | PLNGAV | 884 | 0.719 | 0.394 | 0.14 |
| HLYAQA | | | | | NLYAQA | | | | |
| QTKQVQ | | | | | QSAPVQ | | | | |
| N | | | | | N | | | | |
| PLNGSV | 386 | 0.144 | 1.279 | 972 | PLNGTV | 885 | 0.384 | 0.394 | 0.09 |
| HLYAQA | | | | | HLYAQA | | | | |
| QTGWVQ | | | | | QTGWVP | | | | |
| Z | | | | | N | | | | |
| PLNGAV | 387 | 0.593 | 1.278 | 11.17 | PVDGAV | 886 | 0.792 | 0.394 | 0.23 |
| HLYAQA | | | | | HLYAQA | | | | |
| QLGQVQ | | | | | QTGSVQ | | | | |
| N | | | | | N | | | | |
| SLNGAV | 388 | 0.684 | 1.277 | 19.35 | PLNGAV | 887 | 0.261 | 0.393 | 0.09 |
| HLYAQA | | | | | HLYDQA | | | | |
| QTGRVQ | | | | | QTAAVQ | | | | |
| Z | | | | | N | | | | |
| PLDGAV | 389 | 0.647 | 1.275 | 263.1 | PLKGAV | 888 | 0.352 | 0.392 | 0.09 |
| HLYAQA | | | | | HLYAQA | | | | |
| QTGWVQ | | | | | QTSTVQ | | | | |
| N | | | | | N | | | | |
| PLNGSV | 390 | 0.181 | 1.273 | 0.19 | PLNGAV | 889 | 0.730 | 0.392 | 0.14 |
| HLYAQA | | | | | HLYAQA | | | | |
| QTGWAQ | | | | | QTGPVQ | | | | |
| N | | | | | T | | | | |
| TLNGAV | 391 | 0.405 | 1.266 | 0.09 | PLNGAV | 890 | 0.432 | 0.392 | 0.14 |
| HLYAQA | | | | | HLYAQA | | | | |
| QVASVQ | | | | | QTVGVQ | | | | |
| N | | | | | K | | | | |
| PLNGAV | 392 | 0.733 | 1.261 | 2.85 | PLNGAV | 891 | 0.451 | 0.391 | 0.14 |
| HLYAQA | | | | | NLYAQA | | | | |
| QNMQVQ | | | | | QTSTVQ | | | | |
| N | | | | | N | | | | |
| PLNGAV | 393 | 0.863 | 1.258 | 3.18 | PLNGAV | 892 | 0.414 | 0.390 | 0.09 |
| HLYAQA | | | | | HLYARA | | | | |
| QNVQVQ | | | | | QTVSVQ | | | | |
| N | | | | | N | | | | |
| PLNGAV | 394 | 0.299 | 1.250 | 0.14 | PLNGAV | 893 | 0.431 | 0.390 | 0.09 |
| HLYAQA | | | | | HLYAQA | | | | |
| QTGSVQ | | | | | QTASGQ | | | | |
| I | | | | | N | | | | |
| PLNGTV | 395 | 0.840 | 1.249 | 15.75 | PLNGAV | 894 | 0.631 | 0.390 | 20.28 |
| HLYAQA | | | | | HLYAQA | | | | |
| QTGGVQ | | | | | QSVSVQ | | | | |
| N | | | | | N | | | | |
| PLNGAV | 396 | 0.805 | 1.248 | 0.09 | PLNGAV | 895 | 0.724 | 0.389 | 0.09 |
| HLYAKA | | | | | HLYAQA | | | | |
| QVSPVQ | | | | | QSGPVQ | | | | |
| N | | | | | T | | | | |
| PLNGAV | 397 | 0.765 | 1.241 | 6.4 | QLNGAV | 896 | 0.578 | 0.389 | 0.47 |
| HLYAQA | | | | | HLYAQA | | | | |
| QRGGVQ | | | | | QTGWGQ | | | | |
| N | | | | | N | | | | |
| PLNGGV | 398 | 0.335 | 1.240 | 25.94 | PLNGAV | 897 | 0.670 | 0.386 | 0.09 |
| HLYAQA | | | | | HLYAQA | | | | |
| QTGLVQ | | | | | QVMSVK | | | | |
| N | | | | | N | | | | |
| PLNGAV | 399 | 0.542 | 1.237 | 33.32 | PLNGTV | 898 | 0.557 | 0.386 | 0.09 |
| HLYAQA | | | | | HLYAKA | | | | |
| QVMSVQ | | | | | QTGSVQ | | | | |
| N | | | | | N | | | | |
| PLNGAV | 400 | 0.401 | 1.235 | 15.05 | PLNGAV | 899 | 0.527 | 0.385 | 0.09 |
| HLYAQA | | | | | NLYAQA | | | | |
| QTTGVQ | | | | | QTSLVQ | | | | |
| N | | | | | N | | | | |
| PLNGAV | 401 | 0.375 | 1.229 | 0.09 | PINGAV | 900 | 0.867 | 0.385 | 0.14 |
| HLYAQA | | | | | HLYAQA | | | | |
| QLSPVQ | | | | | QTLSVQ | | | | |
| K | | | | | N | | | | |
| SLNGAV | 402 | 0.677 | 1.226 | 0.09 | PLNGAV | 901 | 0.458 | 0.384 | 0.09 |
| HLYAQA | | | | | HLYAQA | | | | |
| QTAAVQ | | | | | QLGSVQ | | | | |
| N | | | | | K | | | | |
| PINGAV | 403 | 0.351 | 1.223 | 0.14 | TLNGAV | 902 | 0.728 | 0.383 | 0.09 |
| HLYAQA | | | | | HLYAQA | | | | |
| QTSPVQ | | | | | QTAMVQ | | | | |
| N | | | | | N | | | | |
| PLNGAV | 404 | 0.691 | 1.220 | 15.28 | PLNGAV | 903 | 0.775 | 0.383 | 0.09 |
| HLYAQA | | | | | HLYAQA | | | | |
| QLSRVQ | | | | | HLSSVQ | | | | |
| N | | | | | N | | | | |
| PLNGAV | 405 | 0.259 | 1.213 | 0.09 | PLNGAV | 904 | 0.552 | 0.380 | 0.09 |
| HLYAQA | | | | | HLYAKA | | | | |
| QVSSVQ | | | | | QVMSVQ | | | | |
| K | | | | | N | | | | |
| PLNGAV | 406 | 0.768 | 1.212 | 0.09 | PLNGAV | 905 | 0.569 | 0.380 | 0.09 |
| HLYAQA | | | | | HLYAQA | | | | |
| QTAPVQ | | | | | QTGLVL | | | | |
| T | | | | | N | | | | |
| PLNGAV | 407 | 0.740 | 1.206 | 7.9 | PLNGGV | 906 | 0.491 | 0.378 | 1.03 |
| HLYAQA | | | | | HLYAQA | | | | |
| QMAPVQ | | | | | QTGWVQ | | | | |
| N | | | | | K | | | | |
| PLNGAV | 408 | 0.499 | 1.202 | 7.06 | PLNGAV | 907 | 0.250 | 0.378 | 0.61 |
| HLYAQA | | | | | HLYAKA | | | | |
| QILGVQ | | | | | QTGWVQ | | | | |
| N | | | | | K | | | | |
| PLNGAV | 409 | 0.216 | 1.198 | 0.14 | PLNGAV | 908 | 0.482 | 0.378 | 0.09 |
| HLYAQA | | | | | LLYAQA | | | | |
| QTASVQ | | | | | QTASVQ | | | | |
| T | | | | | N | | | | |
| PLNGAV | 410 | 0.267 | 1.196 | 0.23 | PLNGDV | 909 | 0.507 | 0.378 | 0.09 |
| HLYAQA | | | | | HLYAQA | | | | |
| QTGSLQ | | | | | QTMAVQ | | | | |
| N | | | | | N | | | | |
| PLNGAV | 411 | 0.638 | 1.196 | 28.79 | PLNGAV | 910 | 0.258 | 0.377 | 0.09 |
| HLYAQA | | | | | HLYAKA | | | | |
| QTGTVQ | | | | | QTAAVQ | | | | |
| N | | | | | N | | | | |
| PLNGGV | 412 | 0.578 | 1.193 | 565.1 | PLNGAV | 911 | 0.549 | 0.377 | 763.5 |
| HLYAQA | | | | | HLYAQA | | | | |
| QTGWVQ | | | | | QTGLVQ | | | | |
| N | | | | | N | | | | |
| PLNGAV | 413 | 0.237 | 1.183 | 0.89 | PLNGDV | 912 | 0.699 | 0.376 | 0.14 |
| HLYAQA | | | | | HLYAQA | | | | |
| QTGSVQ | | | | | QSSPVQ | | | | |
| T | | | | | N | | | | |
| PLNGAV | 414 | 0.353 | 1.171 | 0.09 | PLNGAV | 913 | 0.832 | 0.376 | 14.96 |
| HLYAQA | | | | | HLYAQA | | | | |
| QTSSVQ | | | | | QTSNVQ | | | | |
| T | | | | | N | | | | |
| PLNGAV | 415 | 0.414 | 1.167 | 10.84 | ALDGAV | 914 | 0.702 | 0.376 | 0.47 |
| HLYAQA | | | | | HLYAQA | | | | |
| QTSHVQ | | | | | QTGSVQ | | | | |
| N | | | | | N | | | | |
| PLNGGV | 416 | 0.561 | 1.166 | 0.28 | PLNGAV | 915 | 0.848 | 0.375 | 0.09 |
| HLYAQA | | | | | HLYSQA | | | | |
| QTGWDQ | | | | | QTSSVQ | | | | |
| N | | | | | N | | | | |
| PLNGAV | 417 | 0.753 | 1.165 | 3.55 | PLKGAV | 916 | 0.390 | 0.373 | 0.19 |
| HLYAQA | | | | | HLYAQA | | | | |
| QRIGVQ | | | | | QLSPVQ | | | | |
| N | | | | | N | | | | |
| TLNGAV | 418 | 0.771 | 1.164 | 0.09 | LLNGAV | 917 | 0.349 | 0.373 | 0.09 |
| HLYAQA | | | | | HLYAQA | | | | |
| QTTSVQ | | | | | QTVSVQ | | | | |
| N | | | | | N | | | | |
| PLNGAV | 419 | 0.347 | 1.158 | 0.14 | PLNGGV | 918 | 0.571 | 0.373 | 0.37 |
| HLYAQA | | | | | HLYAQA | | | | |
| QTGWGQ | | | | | QTGWVQ | | | | |
| T | | | | | I | | | | |
| PLNGAV | 420 | 0.549 | 1.158 | 14.3 | PLNGAV | 919 | 0.863 | 0.372 | 0.14 |
| HLYAQA | | | | | HLYSQA | | | | |
| QSAMVQ | | | | | QTGWVR | | | | |
| N | | | | | N | | | | |
| PLNGAV | 421 | 0.510 | 1.156 | 23.23 | PLNGAV | 920 | 0.426 | 0.371 | 0.09 |
| HLYAQA | | | | | HLYAQA | | | | |
| QTSMVQ | | | | | QTGWGH | | | | |
| N | | | | | N | | | | |
| PLNGAV | 422 | 0.781 | 1.148 | 13.09 | PHNGAV | 921 | 0.849 | 0.371 | 0.09 |
| HLYAQA | | | | | HLYAQA | | | | |
| QSMGVQ | | | | | QTTSVQ | | | | |
| N | | | | | N | | | | |
| PLNGAV | 423 | 0.568 | 1.145 | 27.62 | PLNRAV | 922 | 0.436 | 0.371 | 0.09 |
| HLYAQA | | | | | HLYAQA | | | | |
| QSMSVQ | | | | | QTGWVQ | | | | |
| N | | | | | K | | | | |
| PLNGAV | 424 | 0.442 | 1.145 | 94.03 | PLNGAV | 923 | 0.509 | 0.371 | 0.09 |
| HLYAQA | | | | | HLYDQA | | | | |
| QTSSVQ | | | | | QRASVQ | | | | |
| N | | | | | N | | | | |
| PLNGAV | 425 | 0.620 | 1.143 | 30.52 | PLNGAV | 924 | 0.800 | 0.370 | 584.7 |
| HLYAQA | | | | | HLYSQA | | | | |
| QTSGVQ | | | | | QTGWVQ | | | | |
| N | | | | | N | | | | |
| PLNGAV | 426 | 0.557 | 1.141 | 22.9 | PLNGSV | 925 | 0.401 | 0.370 | 0.42 |
| HLYAQA | | | | | HLYAQA | | | | |
| QTGAVQ | | | | | QTGTVQ | | | | |
| N | | | | | N | | | | |
| PLNGAV | 427 | 0.627 | 1.140 | 14.02 | PLNGAV | 926 | 0.752 | 0.370 | 0.09 |
| HLYAQA | | | | | HLYDQA | | | | |
| QVNSVQ | | | | | QTSGVQ | | | | |
| N | | | | | N | | | | |
| PLNGAV | 428 | 0.804 | 1.139 | 12.48 | PLNGAV | 927 | 0.389 | 0.368 | 0.19 |
| HLYAQA | | | | | HLYAQA | | | | |
| QVAGVQ | | | | | KTGWFQ | | | | |
| N | | | | | N | | | | |
| PLNGAV | 429 | 0.710 | 1.139 | 27.01 | KLDGAV | 928 | 0.763 | 0.367 | 0.14 |
| HLYAQA | | | | | HLYAQA | | | | |
| QIGSVQ | | | | | QTGLVQ | | | | |
| N | | | | | N | | | | |
| PLDGAV | 430 | 0.818 | 1.132 | 9.49 | PLNGAV | 929 | 0.581 | 0.363 | 9.39 |
| HVYAQA | | | | | HLYAQA | | | | |
| QTGWVQ | | | | | QSQMVQ | | | | |
| N | | | | | N | | | | |
| TLNGAV | 431 | 0.474 | 1.131 | 0.09 | TLNGAV | 930 | 0.706 | 0.363 | 0.09 |
| HLYAQA | | | | | HLYAQA | | | | |
| QTTPVQ | | | | | QTSVVQ | | | | |
| N | | | | | N | | | | |
| PLNGAV | 432 | 0.442 | 1.131 | 41.64 | PLNGAV | 931 | 0.449 | 0.361 | 0.09 |
| HLYAQA | | | | | HLYAKA | | | | |
| QLGSVQ | | | | | QTGTVQ | | | | |
| N | | | | | N | | | | |
| PLNGAV | 433 | 0.807 | 1.130 | 7.06 | PLNGAV | 932 | 0.440 | 0.361 | 0.09 |
| HLYAQA | | | | | HLYAQA | | | | |
| QVNGVQ | | | | | QTVSVH | | | | |
| N | | | | | N | | | | |
| QLNGAV | 434 | 0.857 | 1.127 | 0.19 | PLNGAV | 933 | 0.559 | 0.361 | 0.19 |
| HLYAQA | | | | | HLYAQA | | | | |
| QLSSVQ | | | | | QLSSVQ | | | | |
| N | | | | | T | | | | |
| PLNGAV | 435 | 0.652 | 1.127 | 7.62 | PVNGAV | 934 | 0.791 | 0.359 | 0.28 |
| HLYAQA | | | | | HLYAQA | | | | |
| QLTAVQ | | | | | QTGWFQ | | | | |
| N | | | | | N | | | | |
| PLNGAV | 436 | 0.892 | 1.125 | 3.22 | PLNGAV | 935 | 0.266 | 0.356 | 0.09 |
| HLYAQA | | | | | HLYAQA | | | | |
| QVQNVQ | | | | | QTASVR | | | | |
| N | | | | | N | | | | |
| PLNGAV | 437 | 0.737 | 1.117 | 14.63 | PLNGDV | 936 | 0.410 | 0.355 | 0.09 |
| HLYAQA | | | | | HLYAQA | | | | |
| QTKSVQ | | | | | QTSTVQ | | | | |
| N | | | | | N | | | | |
| PLNGAV | 438 | 0.847 | 1.117 | 16.64 | PLNGAV | 937 | 0.477 | 0.354 | 0.09 |
| HLYAQA | | | | | HLYAKA | | | | |
| QSVGVQ | | | | | QLGSVQ | | | | |
| N | | | | | N | | | | |
| QLNGAV | 439 | 0.413 | 1.116 | 0.09 | PINGAV | 938 | 0.774 | 0.354 | 0.09 |
| HLYAQA | | | | | HLYAQA | | | | |
| QLGPVQ | | | | | QLVPVQ | | | | |
| N | | | | | N | | | | |
| PLNGAV | 440 | 0.274 | 1.107 | 0.47 | PLNGAV | 939 | 0.833 | 0.354 | 0.09 |
| HLYAQA | | | | | HHYAQA | | | | |
| QTAWVQ | | | | | QLGPVQ | | | | |
| T | | | | | N | | | | |
| PLNGAV | 441 | 0.651 | 1.099 | 11.54 | PLNGAV | 940 | 0.387 | 0.353 | 0.19 |
| HLYAQA | | | | | HLYAPA | | | | |
| QNASVQ | | | | | QTGWVQ | | | | |
| N | | | | | H | | | | |
| PLNGAV | 442 | 0.661 | 1.098 | 45.57 | PLNGAV | 941 | 0.696 | 0.353 | 0.56 |
| HLYAQA | | | | | HLYAQA | | | | |
| QTSAVQ | | | | | QVPAVQ | | | | |
| N | | | | | N | | | | |
| ALNGAV | 443 | 0.530 | 1.097 | 0.09 | PLNGAV | 942 | 0.774 | 0.353 | 0.09 |
| HLYAQA | | | | | NLYAQA | | | | |
| QLAAVQ | | | | | QRLSVQ | | | | |
| N | | | | | N | | | | |
| PLNGAV | 444 | 0.721 | 1.097 | 0.09 | PLNGAV | 943 | 0.534 | 0.352 | 0.23 |
| HLYDQA | | | | | HLYAQA | | | | |
| QVSPVQ | | | | | QTGRVQ | | | | |
| N | | | | | K | | | | |
| PLNGAV | 445 | 0.442 | 1.095 | 0.09 | PLNGAV | 944 | 0.370 | 0.352 | 0.09 |
| HLYAQA | | | | | LLYAQA | | | | |
| QTMSVK | | | | | QTVSVQ | | | | |
| N | | | | | N | | | | |
| PLNGAV | 446 | 0.650 | 1.092 | 7.71 | PLNGAV | 945 | 0.510 | 0.351 | 11.4 |
| HLYAQA | | | | | HLYAQA | | | | |
| QTANVQ | | | | | QAMSVQ | | | | |
| N | | | | | N | | | | |
| PLNGAV | 447 | 0.358 | 1.092 | 5.19 | PLNGAV | 946 | 0.451 | 0.351 | 0.23 |
| HLYAQA | | | | | HLYAQA | | | | |
| QTGWFQ | | | | | QTAWVH | | | | |
| N | | | | | N | | | | |
| PLNGNV | 448 | 0.392 | 1.090 | 74.03 | PLNGAV | 947 | 0.615 | 0.350 | 0.09 |
| HLYAQA | | | | | HLYAQP | | | | |
| QTGWVQ | | | | | QTGWVR | | | | |
| N | | | | | N | | | | |
| PLNGSV | 449 | 0.195 | 1.087 | 0.23 | PLNGAV | 948 | 0.548 | 0.350 | 0.09 |
| HLYAQA | | | | | HLYAQE | | | | |
| QTGWVQ | | | | | QTGWLQ | | | | |
| S | | | | | N | | | | |
| PLNGAV | 450 | 0.279 | 1.081 | 0.09 | PLNGAV | 949 | 0.535 | 0.348 | 0.09 |
| HLYAQA | | | | | HLYAKA | | | | |
| QTGGVL | | | | | QTSAVQ | | | | |
| N | | | | | N | | | | |
| PLNGAV | 451 | 0.888 | 1.081 | 16.45 | PLNGSV | 950 | 0.126 | 0.348 | 0.98 |
| HLYAQA | | | | | HLYAQA | | | | |
| QLTSVQ | | | | | QTVWVQ | | | | |
| N | | | | | N | | | | |
| PLNGAV | 452 | 0.176 | 1.079 | 19.3 | PLNGTV | 951 | 0.330 | 0.347 | 0.33 |
| HLYAQA | | | | | HLYAQA | | | | |
| QTGWVQ | | | | | QTGWFQ | | | | |
| T | | | | | N | | | | |
| PLNGAV | 453 | 0.804 | 1.078 | 10.7 | PLNGAV | 952 | 0.375 | 0.347 | 0.23 |
| HLYAQA | | | | | NLYAQA | | | | |
| QRSQVQ | | | | | QVASVQ | | | | |
| N | | | | | N | | | | |
| PLNGAV | 454 | 0.840 | 1.074 | 0.09 | PLNGAV | 953 | 0.674 | 0.347 | 0.19 |
| HIYAQA | | | | | HLYAQA | | | | |
| QVSPVQ | | | | | QTGLVR | | | | |
| N | | | | | N | | | | |
| PLNGSV | 455 | 0.130 | 1.062 | 0.37 | PLNGAV | 954 | 0.587 | 0.346 | 0.09 |
| HLYAQA | | | | | NLYAQA | | | | |
| QTGWFQ | | | | | QLSTVQ | | | | |
| N | | | | | N | | | | |
| PLNGAV | 456 | 0.863 | 1.060 | 3.13 | PLNGAV | 955 | 0.721 | 0.346 | 0.09 |
| HLYAQA | | | | | HLYAKA | | | | |
| QRIPVQ | | | | | QTQPVQ | | | | |
| N | | | | | N | | | | |
| QLNGAV | 457 | 0.718 | 1.054 | 0.09 | PLNGTV | 956 | 0.382 | 0.346 | 0.33 |
| HLYAQA | | | | | HLYAQA | | | | |
| QLVPVQ | | | | | QTGWGQ | | | | |
| N | | | | | N | | | | |
| PLNGAV | 458 | 0.198 | 1.052 | 2.06 | PLNGAV | 957 | 0.453 | 0.345 | 0.09 |
| HLYAQA | | | | | HLYAQA | | | | |
| QTGWVQ | | | | | QRTLVQ | | | | |
| D | | | | | T | | | | |
| PLNGAV | 459 | 0.357 | 1.051 | 0.09 | PLNGAV | 958 | 0.157 | 0.345 | 0.61 |
| HLYAKA | | | | | HLYAQA | | | | |
| QVSSVQ | | | | | QTGSIQ | | | | |
| N | | | | | N | | | | |
| PLNGAV | 460 | 0.721 | 1.051 | 11.54 | QLNGAV | 959 | 0.574 | 0.344 | 1.4 |
| HLYAQA | | | | | HLYAQA | | | | |
| QRVEVQ | | | | | QTGWDQ | | | | |
| N | | | | | N | | | | |
| PLNGAV | 461 | 0.553 | 1.048 | 56.6 | PLNGAV | 960 | 0.599 | 0.344 | 0.14 |
| HLYAQA | | | | | HLYAQA | | | | |
| QVLPVQ | | | | | QLSSVK | | | | |
| N | | | | | N | | | | |
| PLNGSV | 462 | 0.194 | 1.044 | 0.28 | QLNGAV | 961 | 0.451 | 0.343 | 1.45 |
| HLYAQA | | | | | HLYAQA | | | | |
| QTGWVR | | | | | QTVSVQ | | | | |
| N | | | | | N | | | | |
| PLNGTV | 463 | 0.648 | 1.040 | 0.09 | PLNGAV | 962 | 0.726 | 0.343 | 0.09 |
| HLYAQA | | | | | HLYAQA | | | | |
| QSSPVQ | | | | | QTLQVQ | | | | |
| N | | | | | K | | | | |
| PLNGAV | 464 | 0.301 | 1.039 | 0.42 | TLNGAV | 963 | 0.573 | 0.343 | 0.14 |
| HLYAQA | | | | | HLYAQA | | | | |
| QTGGVQ | | | | | QVLPVQ | | | | |
| K | | | | | N | | | | |
| PLNGAV | 465 | 0.727 | 1.039 | 14.63 | PLNGAV | 964 | 0.756 | 0.342 | 0.09 |
| HLYAQA | | | | | HLYAQA | | | | |
| QSVAVQ | | | | | QVGSVK | | | | |
| N | | | | | N | | | | |
| QLNGAV | 466 | 0.815 | 1.030 | 0.09 | PLNGAV | 965 | 0.744 | 0.341 | 0.09 |
| HLYAQA | | | | | HLYAQA | | | | |
| QLSGVQ | | | | | QLAPLQ | | | | |
| N | | | | | N | | | | |
| PLNGAV | 467 | 0.384 | 1.026 | 11.26 | QLNGAV | 966 | 0.716 | 0.341 | 0.09 |
| HLYAQA | | | | | HLYAQA | | | | |
| QLQQVQ | | | | | QTGSVK | | | | |
| N | | | | | N | | | | |
| PLNGAV | 468 | 0.547 | 1.025 | 32.06 | PLNGAV | 967 | 0.620 | 0.341 | 0.09 |
| HLYAQA | | | | | HHYAQA | | | | |
| QLSTVQ | | | | | QRSTVQ | | | | |
| N | | | | | N | | | | |
| PLNGAV | 469 | 0.813 | 1.025 | 18.09 | QLNGSV | 968 | 0.266 | 0.341 | 0.23 |
| HLYAQA | | | | | HLYAQA | | | | |
| QSAAVQ | | | | | QTGRVQ | | | | |
| N | | | | | N | | | | |
| SLNGAV | 470 | 0.774 | 1.014 | 0.14 | TLNGAV | 969 | 0.818 | 0.340 | 0.09 |
| HLYAQA | | | | | HLYAQA | | | | |
| QVSAVQ | | | | | QVKPVQ | | | | |
| N | | | | | N | | | | |
| PLNGAV | 471 | 0.432 | 1.013 | 20.47 | PLNGTV | 970 | 0.784 | 0.339 | 24.58 |
| HLYAQA | | | | | HLYAQA | | | | |
| QRTSVQ | | | | | QTGLVQ | | | | |
| N | | | | | N | | | | |
| PLNGAV | 472 | 0.877 | 1.012 | 5.47 | PLNGAV | 971 | 0.396 | 0.338 | 0.65 |
| HLYAQA | | | | | HLYAQA | | | | |
| QTQNVQ | | | | | QTPGVQ | | | | |
| N | | | | | N | | | | |
| PLNGAV | 473 | 0.877 | 1.007 | 4.81 | PINGAV | 972 | 0.713 | 0.338 | 0.28 |
| HLYAQA | | | | | HLYAQA | | | | |
| QVNAVQ | | | | | QTSSVQ | | | | |
| N | | | | | N | | | | |
| PLNGAV | 474 | 0.585 | 1.006 | 0.09 | PLNGAV | 973 | 0.428 | 0.337 | 0.09 |
| HLYAQA | | | | | HLYAQA | | | | |
| QTVAVQ | | | | | QLSAVQ | | | | |
| T | | | | | T | | | | |
| PLNGAV | 475 | 0.487 | 1.005 | 20.75 | PLNGAV | 974 | 0.446 | 0.337 | 0.47 |
| HLYAQA | | | | | HLYSQA | | | | |
| QLMSVQ | | | | | QTGWDQ | | | | |
| N | | | | | N | | | | |
| PLNGAV | 476 | 0.179 | 1.000 | 15655 | PLNCAV | 975 | 0.856 | 0.336 | 14.39 |
| HLYAQA | | | | | HLYAQA | | | | |
| QTGWVQ | | | | | QTGWVQ | | | | |
| N | | | | | N | | | | |
| PLNGAV | 477 | 0.354 | 0.999 | 0.09 | PLNGAV | 976 | 0.624 | 0.336 | 0.14 |
| HLYAHA | | | | | NLYAQA | | | | |
| QTGWVQ | | | | | QTMPVQ | | | | |
| H | | | | | N | | | | |
| PLNGAV | 478 | 0.283 | 0.997 | 7.48 | PLNGAV | 977 | 0.283 | 0.335 | 0.09 |
| HLYAQA | | | | | HLYSQA | | | | |
| QTGWVQ | | | | | QTVAVQ | | | | |
| H | | | | | N | | | | |
| PLNGAV | 479 | 0.408 | 0.993 | 0.09 | PLNGTV | 978 | 0.366 | 0.334 | 0.79 |
| HHYAQA | | | | | HLYAQA | | | | |
| QTMSVQ | | | | | QTGWVK | | | | |
| N | | | | | N | | | | |
| PLNGAV | 480 | 0.228 | 0.978 | 3.08 | PLNGGV | 979 | 0.465 | 0.333 | 0.37 |
| HLYAQA | | | | | HLYAQA | | | | |
| QTGWVQ | | | | | QTGWVH | | | | |
| S | | | | | N | | | | |
| PLNGAV | 481 | 0.409 | 0.976 | 0.09 | QLNGAV | 980 | 0.558 | 0.332 | 1.59 |
| HLYDQA | | | | | HLYAQA | | | | |
| QTTPVQ | | | | | QTGWVQ | | | | |
| N | | | | | K | | | | |
| PLNGAV | 482 | 0.837 | 0.973 | 3.69 | PLNGAV | 981 | 0.524 | 0.331 | 0.09 |
| HLYAQA | | | | | QLYAQA | | | | |
| QTMMVQ | | | | | QTLAVQ | | | | |
| N | | | | | N | | | | |
| PLNGAV | 483 | 0.293 | 0.971 | 0.14 | PLNGTV | 982 | 0.695 | 0.331 | 0.09 |
| HLYAQA | | | | | HLYAQA | | | | |
| QTGGVQ | | | | | QVGSVQ | | | | |
| S | | | | | N | | | | |
| PLNGAV | 484 | 0.499 | 0.969 | 0.14 | PLNGGV | 983 | 0.764 | 0.330 | 17.67 |
| NLYAQA | | | | | HLYAQA | | | | |
| QTSPVQ | | | | | QTGGVQ | | | | |
| N | | | | | N | | | | |
| ALNGAV | 485 | 0.322 | 0.968 | 47.95 | PLKGAV | 984 | 0.474 | 0.330 | 0.09 |
| HLYAQA | | | | | HLYAQA | | | | |
| QTGLVQ | | | | | QVSQVQ | | | | |
| N | | | | | N | | | | |
| ALNGAV | 486 | 0.247 | 0.964 | 0.09 | PLNGAV | 985 | 0.526 | 0.330 | 0.09 |
| HLYAQA | | | | | HHYAQA | | | | |
| QVATVQ | | | | | QTQPVQ | | | | |
| N | | | | | N | | | | |
| PLNGAV | 487 | 0.258 | 0.959 | 0.23 | PLNGAV | 986 | 0.156 | 0.329 | 0.33 |
| HLYAQA | | | | | HLYAQA | | | | |
| QTGSFQ | | | | | PTGWVQ | | | | |
| N | | | | | T | | | | |
| PLNGAV | 488 | 0.422 | 0.958 | 27.01 | PLNGAV | 987 | 0.343 | 0.329 | 0.33 |
| HLYAQA | | | | | NLYAQA | | | | |
| QRSVVQ | | | | | QTMSVQ | | | | |
| N | | | | | N | | | | |
| PLNGAV | 489 | 0.224 | 0.957 | 0.65 | PLNGAV | 988 | 0.681 | 0.329 | 29.16 |
| HLYAQA | | | | | HLYAQA | | | | |
| QTGSVQ | | | | | QTGVVQ | | | | |
| K | | | | | N | | | | |
| PLNGAV | 490 | 0.503 | 0.954 | 16.31 | PLNGAV | 989 | 0.622 | 0.328 | 0.42 |
| HLYAQA | | | | | HLYAQA | | | | |
| QTQLVQ | | | | | QVPQVQ | | | | |
| N | | | | | N | | | | |
| PLNGAV | 491 | 0.252 | 0.954 | 0.19 | PHNGAV | 990 | 0.295 | 0.327 | 0.14 |
| HLYAQA | | | | | HLYAQA | | | | |
| QTGGVH | | | | | QTMSVQ | | | | |
| N | | | | | N | | | | |
| PLNGAV | 492 | 0.328 | 0.953 | 11.92 | QLNGAV | 991 | 0.493 | 0.327 | 0.56 |
| HLYAQA | | | | | HLYAQA | | | | |
| QRTPVQ | | | | | QTGWLQ | | | | |
| N | | | | | N | | | | |
| PLNGAV | 493 | 0.852 | 0.952 | 5.33 | PLNGAV | 992 | 0.118 | 0.327 | 1.03 |
| HLYAQA | | | | | HLYAQA | | | | |
| QRQQVQ | | | | | QTGCVQ | | | | |
| N | | | | | K | | | | |
| PLNGAV | 494 | 0.321 | 0.952 | 0.19 | SLNGAV | 993 | 0.712 | 0.327 | 0.28 |
| HLYAQA | | | | | HLYAQA | | | | |
| QTGSVR | | | | | QTSLVQ | | | | |
| N | | | | | N | | | | |
| PLNGAV | 495 | 0.758 | 0.946 | 11.87 | PLNGAV | 994 | 0.422 | 0.326 | 0.23 |
| HLYAQA | | | | | HLYAQA | | | | |
| QVQGVQ | | | | | QTRWVQ | | | | |
| N | | | | | T | | | | |
| TLNGAV | 496 | 0.854 | 0.942 | 0.14 | ILNGAV | 995 | 0.743 | 0.325 | 0.09 |
| HLYAQA | | | | | HLYAQA | | | | |
| QVSPVQ | | | | | QTVQVQ | | | | |
| N | | | | | N | | | | |
| PLNGAV | 497 | 0.205 | 0.942 | 0.23 | ALNGAV | 996 | 0.762 | 0.324 | 0.09 |
| HLYAQA | | | | | HLYAQA | | | | |
| QPGWVQ | | | | | QKGGVQ | | | | |
| T | | | | | N | | | | |
| PLNGAV | 498 | 0.407 | 0.941 | 17.25 | PLNGAV | 997 | 0.143 | 0.323 | 0.09 |
| HLYAQA | | | | | HLYDQA | | | | |
| QSTQVQ | | | | | QVSGVQ | | | | |
| N | | | | | N | | | | |
| PLNGSV | 499 | 0.247 | 0.939 | 0.09 | PLNGAV | 998 | 0.554 | 0.323 | 0.65 |
| HLYAQA | | | | | HLYAQA | | | | |
| QTGWVE | | | | | QTGLDQ | | | | |
| N | | | | | N | | | | |
| PLNGAV | 500 | 0.267 | 0.934 | 22.29 | PLNGAV | 999 | 0.387 | 0.323 | 0.42 |
| HLYAQA | | | | | PLYAQA | | | | |
| QQSPVQ | | | | | QTGWVQ | | | | |
| N | | | | | T | | | | |
| PLNGAV | 501 | 0.517 | 0.934 | 8.23 | PLNGAV | 1000 | 0.414 | 0.323 | 0.09 |
| HLYAQA | | | | | HLYAQA | | | | |
| QRYSVQ | | | | | QVSSVH | | | | |
| N | | | | | N | | | | |
| PLNGAV | 502 | 0.267 | 0.930 | 9.77 | TLNGAV | 1001 | 0.264 | 0.322 | 0.09 |
| HLYAQA | | | | | HLYAQA | | | | |
| QTGWDQ | | | | | QTNSVQ | | | | |
| N | | | | | N | | | | |
| PLNGAV | 503 | 0.774 | 0.930 | 4.67 | PLNGAG | 1002 | 0.479 | 0.321 | 0.09 |
| HLYAQA | | | | | HLYAQA | | | | |
| QTQRVQ | | | | | QLSPVQ | | | | |
| N | | | | | N | | | | |
| ALNGAV | 504 | 0.330 | 0.926 | 0.23 | HLNGAV | 1003 | 0.532 | 0.321 | 0.19 |
| HLYAQA | | | | | HLYAQA | | | | |
| QTGWVR | | | | | QTGRVQ | | | | |
| N | | | | | N | | | | |
| PLNGAV | 505 | 0.370 | 0.923 | 171 | PLNGAV | 1004 | 0.872 | 0.320 | 0.09 |
| HLYAQP | | | | | HLYDQA | | | | |
| QTGWVQ | | | | | QTAMVQ | | | | |
| N | | | | | N | | | | |
| ALNGAV | 506 | 0.516 | 0.920 | 0.09 | PINGAV | 1005 | 0.646 | 0.320 | 0.33 |
| HLYAQA | | | | | HLYAQA | | | | |
| QVSGVQ | | | | | QTGWVQ | | | | |
| N | | | | | H | | | | |
| PLNGAV | 507 | 0.813 | 0.920 | 17.01 | PLNGAV | 1006 | 0.479 | 0.319 | 0.09 |
| HLYAQA | | | | | QLYAQA | | | | |
| QSAGVQ | | | | | QTLQVQ | | | | |
| N | | | | | N | | | | |
| PLNGAV | 508 | 0.734 | 0.918 | 0.09 | PLNGAV | 1007 | 0.323 | 0.318 | 0.28 |
| HLYAQA | | | | | HLYAQA | | | | |
| QSAPVQ | | | | | QKGWVQ | | | | |
| T | | | | | T | | | | |
| PLNGAV | 509 | 0.499 | 0.914 | 13.97 | PLNGAV | 1008 | 0.592 | 0.318 | 0.09 |
| HLYAQA | | | | | HHYAQA | | | | |
| QRQSVQ | | | | | QSTSVQ | | | | |
| N | | | | | N | | | | |
| PLNGAV | 510 | 0.591 | 0.906 | 20.42 | PLNGYV | 1009 | 0.187 | 0.317 | 2.99 |
| HLYAQA | | | | | HLYAQA | | | | |
| QSQPVQ | | | | | QTGWVQ | | | | |
| N | | | | | N | | | | |
| PLNGAV | 511 | 0.570 | 0.906 | 0.09 | PLNGAV | 1010 | 0.599 | 0.316 | 0.09 |
| HLYAQA | | | | | HIYAQA | | | | |
| QVASVK | | | | | QVQSVQ | | | | |
| N | | | | | N | | | | |
| PLNGAV | 512 | 0.403 | 0.904 | 0.09 | PLIGAV | 1011 | 0.535 | 0.315 | 0.09 |
| HLYAQA | | | | | HLYAQA | | | | |
| QTAWVR | | | | | QLSPVQ | | | | |
| N | | | | | N | | | | |
| SLNGAV | 513 | 0.491 | 0.904 | 0.09 | TLNGAV | 1012 | 0.774 | 0.315 | 0.09 |
| HLYAQA | | | | | HLYAQA | | | | |
| QTMQVQ | | | | | QLLPVQ | | | | |
| N | | | | | N | | | | |
| PLNGAV | 514 | 0.664 | 0.902 | 0.09 | PLNGAV | 1013 | 0.747 | 0.315 | 0.84 |
| NLYAQA | | | | | HLYSQA | | | | |
| QSSPVQ | | | | | QTGWVQ | | | | |
| N | | | | | T | | | | |
| PLNGAV | 515 | 0.318 | 0.901 | 0.23 | ALNGAV | 1014 | 0.444 | 0.315 | 0.14 |
| HLYAQA | | | | | HLYAQA | | | | |
| QTGGGQ | | | | | QTQLVQ | | | | |
| N | | | | | N | | | | |
| PLNGAV | 516 | 0.515 | 0.898 | 0.09 | PLNGAV | 1015 | 0.810 | 0.315 | 34.86 |
| HLYDQA | | | | | HLYAQA | | | | |
| QVASVQ | | | | | QSLPVQ | | | | |
| N | | | | | N | | | | |
| PLNGAV | 517 | 0.709 | 0.897 | 45.94 | PVNGAV | 1016 | 0.890 | 0.314 | 0.09 |
| HLYAQA | | | | | HLYAQA | | | | |
| QSTSVQ | | | | | QTGWVP | | | | |
| N | | | | | N | | | | |
| PLNGTV | 518 | 0.403 | 0.894 | 0.23 | PLNGAV | 1017 | 0.247 | 0.313 | 0.19 |
| HLYAQA | | | | | HLYAQA | | | | |
| QTGWVQ | | | | | KTVSVQ | | | | |
| H | | | | | N | | | | |
| QLNGAV | 519 | 0.718 | 0.892 | 0.42 | PLNGAV | 1018 | 0.645 | 0.313 | 0.09 |
| HLYAQA | | | | | HLYAQA | | | | |
| QTSSVQ | | | | | QTTSVH | | | | |
| N | | | | | N | | | | |
| PLDGAV | 520 | 0.613 | 0.892 | 0.09 | PVNGAV | 1019 | 0.765 | 0.313 | 939 |
| HLYAQA | | | | | HLYAQA | | | | |
| QTGWVH | | | | | QTGWVQ | | | | |
| N | | | | | N | | | | |
| PLNGTV | 521 | 0.397 | 0.888 | 0.33 | PLNGAV | 1020 | 0.476 | 0.313 | 0.09 |
| HLYAQA | | | | | HLYAQA | | | | |
| QTGWVQ | | | | | HTGWVQ | | | | |
| K | | | | | I | | | | |
| PLNGAV | 522 | 0.369 | 0.888 | 20.28 | PLNGAV | 1021 | 0.611 | 0.313 | 0.09 |
| HLYAQA | | | | | RLYAQA | | | | |
| QVLGVQ | | | | | QTASVQ | | | | |
| N | | | | | N | | | | |
| PLNGAV | 523 | 0.640 | 0.887 | 5.7 | PLNGGV | 1022 | 0.646 | 0.313 | 0.09 |
| HLYAQA | | | | | HLYAQA | | | | |
| QNQPVQ | | | | | QTGLVQ | | | | |
| N | | | | | K | | | | |
| PLNGAV | 524 | 0.605 | 0.885 | 21.08 | PLNGAV | 1023 | 0.652 | 0.313 | 0.14 |
| HLYAQA | | | | | NLYAQA | | | | |
| QLGTVQ | | | | | QVMSVQ | | | | |
| N | | | | | N | | | | |
| PLNGAV | 525 | 0.637 | 0.882 | 7.57 | PLNGAV | 1024 | 0.682 | 0.312 | 0.09 |
| HLYAQA | | | | | NLYAQA | | | | |
| QRAGVQ | | | | | QSMSVQ | | | | |
| N | | | | | N | | | | |
| PVNGAV | 526 | 0.837 | 0.882 | 42.25 | PLNGAV | 1025 | 0.513 | 0.312 | 0.09 |
| HLYAQA | | | | | HLYAPA | | | | |
| QTGLVQ | | | | | QTGWGQ | | | | |
| N | | | | | N | | | | |
| PLNGGV | 527 | 0.529 | 0.881 | 0.33 | QLNGAV | 1026 | 0.595 | 0.311 | 0.47 |
| HLYAQA | | | | | HLYAQA | | | | |
| QTGWVQ | | | | | QTGWAQ | | | | |
| H | | | | | N | | | | |
| PLNGAV | 528 | 0.677 | 0.879 | 116.4 | PLNGAV | 1027 | 0.451 | 0.311 | 23.13 |
| HLYAQA | | | | | HLYAQA | | | | |
| QTGRVQ | | | | | QTVWVQ | | | | |
| N | | | | | N | | | | |
| PLNGAV | 529 | 0.266 | 0.876 | 0.56 | QLNGAV | 1028 | 0.742 | 0.311 | 0.09 |
| HLYAQA | | | | | HLYAQA | | | | |
| QTGGVK | | | | | QSVSVQ | | | | |
| N | | | | | N | | | | |
| PLNGAV | 530 | 0.251 | 0.874 | 0.14 | PLNGAV | 1029 | 0.426 | 0.311 | 0.09 |
| HLYAQA | | | | | HLYAQA | | | | |
| QTAWLQ | | | | | QTAQLQ | | | | |
| N | | | | | N | | | | |
| PLNGAV | 531 | 0.269 | 0.870 | 0.14 | PLNGAV | 1030 | 0.453 | 0.311 | 0.09 |
| HLYAQA | | | | | HLYAQA | | | | |
| QTRWVQ | | | | | HVASVQ | | | | |
| K | | | | | N | | | | |
| PLNGAV | 532 | 0.699 | 0.858 | 0.09 | PLNGAV | 1031 | 0.440 | 0.310 | 0.09 |
| HLYAQA | | | | | HLYAKA | | | | |
| QLAPVK | | | | | QRSTVQ | | | | |
| N | | | | | N | | | | |
| PLNGAV | 533 | 0.390 | 0.855 | 0.09 | QLNGAV | 1032 | 0.509 | 0.310 | 1.92 |
| HHYAQA | | | | | HLYAQA | | | | |
| QTSSVQ | | | | | QTGWVK | | | | |
| N | | | | | N | | | | |
| PLNGAV | 534 | 0.402 | 0.851 | 0.14 | PLNGAV | 1033 | 0.281 | 0.310 | 0.28 |
| HLYAQA | | | | | HLYAQA | | | | |
| QTGSVQ | | | | | QTRWVK | | | | |
| D | | | | | N | | | | |
| PLNGAV | 535 | 0.557 | 0.849 | 27.67 | PLNGAV | 1034 | 0.381 | 0.310 | 0.19 |
| HLYAQA | | | | | HLYAQA | | | | |
| QTGMVQ | | | | | QTAWIQ | | | | |
| N | | | | | N | | | | |
| PLNGAV | 536 | 0.647 | 0.848 | 15.14 | PLNGAV | 1035 | 0.393 | 0.310 | 0.09 |
| HLYAQA | | | | | HLYAQA | | | | |
| QLSNVQ | | | | | QLSQVK | | | | |
| N | | | | | N | | | | |
| PLNGAV | 537 | 0.502 | 0.844 | 14.39 | SLNGAV | 1036 | 0.816 | 0.310 | 2284 |
| HLYAQA | | | | | HLYAQA | | | | |
| QRLPVQ | | | | | QTGWVQ | | | | |
| N | | | | | N | | | | |
| PLNGAV | 538 | 0.782 | 0.842 | 7.52 | PLNGAV | 1037 | 0.390 | 0.309 | 0.14 |
| HLYAQA | | | | | HLYAQA | | | | |
| QRQGVQ | | | | | QTSTVK | | | | |
| N | | | | | N | | | | |
| PLNGAV | 539 | 0.563 | 0.839 | 0.09 | PLNGAV | 1038 | 0.654 | 0.309 | 16.82 |
| HLYAQA | | | | | HLYAQA | | | | |
| QSTPVQ | | | | | QALPVQ | | | | |
| T | | | | | N | | | | |
| QLNGNV | 540 | 0.844 | 0.838 | 5.56 | PLNGAV | 1039 | 0.158 | 0.309 | 0.33 |
| HLYAQA | | | | | HLYDQA | | | | |
| QTGWVQ | | | | | QTGWFQ | | | | |
| N | | | | | N | | | | |
| PLNGGV | 541 | 0.559 | 0.835 | 0.84 | PLNGAV | 1040 | 0.527 | 0.309 | 0.09 |
| HLYAQA | | | | | HVYAQA | | | | |
| QTGWVQ | | | | | QTGWFQ | | | | |
| T | | | | | N | | | | |
| PLNGAV | 542 | 0.470 | 0.833 | 0.09 | PINGAV | 1041 | 0.386 | 0.308 | 0.09 |
| HLYAQA | | | | | HLYAQA | | | | |
| QTTPVK | | | | | QVGSVQ | | | | |
| N | | | | | N | | | | |
| PLNGAV | 543 | 0.700 | 0.832 | 18.65 | PLNGSV | 1042 | 0.418 | 0.308 | 0.14 |
| HLYAQA | | | | | HLYAQA | | | | |
| QRLTVQ | | | | | QTTGVQ | | | | |
| N | | | | | N | | | | |
| PLNGAV | 544 | 0.373 | 0.830 | 7.52 | PLNGAV | 1043 | 0.874 | 0.307 | 0.09 |
| HLYAQA | | | | | HLYAKA | | | | |
| QTLRVQ | | | | | QASPVQ | | | | |
| N | | | | | N | | | | |
| PLNGAV | 545 | 0.742 | 0.830 | 7.34 | PLNGSV | 1044 | 0.192 | 0.307 | 0.09 |
| HLYAQA | | | | | HLYAQA | | | | |
| QSAFVQ | | | | | QKAWVQ | | | | |
| N | | | | | N | | | | |
| SLNGAV | 546 | 0.586 | 0.824 | 0.09 | SLNGAV | 1045 | 0.270 | 0.307 | 0.09 |
| HLYAQA | | | | | HLYAQA | | | | |
| QTNSVQ | | | | | QVLGVQ | | | | |
| N | | | | | N | | | | |
| PLNGAV | 547 | 0.658 | 0.824 | 6.92 | PLNGAV | 1046 | 0.827 | 0.306 | 0.09 |
| HLYAQA | | | | | HLYAQE | | | | |
| QSVQVQ | | | | | QLMPVQ | | | | |
| N | | | | | N | | | | |
| PLDGAV | 548 | 0.568 | 0.823 | 0.09 | ALNGAV | 1047 | 0.872 | 0.306 | 18.51 |
| HLYAQA | | | | | HLYAQS | | | | |
| QTAAVQ | | | | | QTGWVQ | | | | |
| N | | | | | N | | | | |
| SLNGAV | 549 | 0.532 | 0.821 | 0.09 | PLNGYV | 1048 | 0.508 | 0.305 | 0.09 |
| HLYAQA | | | | | HLYDQA | | | | |
| QLMPVQ | | | | | QTGWVQ | | | | |
| N | | | | | N | | | | |
| PLDGAV | 550 | 0.621 | 0.820 | 0.47 | PLNGAV | 1049 | 0.505 | 0.305 | 0.09 |
| HLYAQA | | | | | HLYAQA | | | | |
| QTGWVQ | | | | | QTSMVQ | | | | |
| T | | | | | T | | | | |
| PLNGAV | 551 | 0.672 | 0.819 | 49.77 | QLNGAV | 1050 | 0.588 | 0.304 | 0.14 |
| HLYAQA | | | | | HLYAQA | | | | |
| QLLPVQ | | | | | QISSVQ | | | | |
| N | | | | | N | | | | |
| PLNGAV | 552 | 0.738 | 0.815 | 6.64 | PVNGAV | 1051 | 0.723 | 0.304 | 1.12 |
| HLYAQA | | | | | HLYAQA | | | | |
| QRTQVQ | | | | | QTGWVQ | | | | |
| N | | | | | T | | | | |
| PLNGAV | 553 | 0.254 | 0.813 | 0.79 | PLNGAV | 1052 | 0.713 | 0.303 | 0.09 |
| HLYAQA | | | | | HLYAQE | | | | |
| QTGSDQ | | | | | QTATVQ | | | | |
| N | | | | | N | | | | |
| PLNGAV | 554 | 0.430 | 0.808 | 0.09 | PLNGAV | 1053 | 0.352 | 0.302 | 0.09 |
| HLYAQA | | | | | YLYAQA | | | | |
| QVASDQ | | | | | QTVSVQ | | | | |
| N | | | | | N | | | | |
| PLNGAV | 555 | 0.744 | 0.805 | 0.09 | PLNGNV | 1054 | 0.521 | 0.302 | 0.09 |
| NLYAQA | | | | | HLYAQA | | | | |
| QLAPVQ | | | | | QTASVQ | | | | |
| N | | | | | N | | | | |
| PLNGSV | 556 | 0.160 | 0.804 | 0.37 | PLNGYV | 1055 | 0.556 | 0.301 | 0.09 |
| HLYAQA | | | | | HLYAQA | | | | |
| QTGWLQ | | | | | QTGLVQ | | | | |
| N | | | | | N | | | | |
| PLNGTV | 557 | 0.253 | 0.801 | 500.6 | PLNGAV | 1056 | 0.402 | 0.301 | 0.14 |
| HLYAQA | | | | | HLYDQA | | | | |
| QTGWVQ | | | | | QTLSVQ | | | | |
| N | | | | | N | | | | |
| PLNGAV | 558 | 0.857 | 0.801 | 0.14 | PLNGAV | 1057 | 0.499 | 0.300 | 0.14 |
| HLYAQA | | | | | HLYAQA | | | | |
| QVSPVK | | | | | QTSSVQ | | | | |
| N | | | | | H | | | | |
| PLNGAV | 559 | 0.480 | 0.801 | 1.22 | SLNGAV | 1058 | 0.737 | 0.300 | 0.19 |
| HLYAQA | | | | | HLYAQA | | | | |
| QLPPVQ | | | | | QTGWVP | | | | |
| N | | | | | N | | | | |
| PLNGAV | 560 | 0.521 | 0.794 | 0.14 | PLNGAV | 1059 | 0.839 | 0.300 | 0.09 |
| HLYAQA | | | | | HLYAQA | | | | |
| QSSPVQ | | | | | QTAMVK | | | | |
| T | | | | | N | | | | |
| PLNGAV | 561 | 0.585 | 0.790 | 8.88 | TLNGAV | 1060 | 0.700 | 0.299 | 0.14 |
| HLYAQA | | | | | HLYAQA | | | | |
| QAQPVQ | | | | | QRLSVQ | | | | |
| N | | | | | N | | | | |
| PLNGDV | 562 | 0.434 | 0.789 | 0.09 | ALNGAV | 1061 | 0.341 | 0.297 | 1.64 |
| HLYAQA | | | | | HLYAQA | | | | |
| QTTSVQ | | | | | QTGWVQ | | | | |
| N | | | | | K | | | | |
| PLNGAV | 563 | 0.392 | 0.788 | 0.75 | PLNGAV | 1062 | 0.722 | 0.297 | 0.19 |
| HLYAQA | | | | | HLYAKA | | | | |
| QTPQVQ | | | | | QTALVQ | | | | |
| N | | | | | N | | | | |
| PLNGAV | 564 | 0.571 | 0.787 | 29.35 | QLNGAV | 1063 | 0.743 | 0.297 | 0.23 |
| HLYAQA | | | | | HLYAQA | | | | |
| QTSWVQ | | | | | QTAKVQ | | | | |
| N | | | | | N | | | | |
| PLNGAV | 565 | 0.254 | 0.784 | 0.51 | PLNGAD | 1064 | 0.744 | 0.297 | 0.14 |
| HLYAQA | | | | | HLYAQA | | | | |
| QTGGDQ | | | | | QTTSVQ | | | | |
| N | | | | | N | | | | |
| PLNGAV | 566 | 0.547 | 0.782 | 0.14 | PLNGAV | 1065 | 0.259 | 0.297 | 0.93 |
| HLYAQA | | | | | HLYAQA | | | | |
| QLSPVK | | | | | QTPWVQ | | | | |
| N | | | | | N | | | | |
| PLNGAV | 567 | 0.603 | 0.782 | 0.09 | PLNGTV | 1066 | 0.324 | 0.296 | 0.28 |
| HLYAQA | | | | | HLYAQA | | | | |
| QSSPVK | | | | | QTGWIQ | | | | |
| N | | | | | N | | | | |
| PLNGAV | 568 | 0.727 | 0.781 | 8.41 | PLNGAV | 1067 | 0.286 | 0.296 | 0.23 |
| HLYAQA | | | | | HLYAQA | | | | |
| QNTTVQ | | | | | HTGWVQ | | | | |
| N | | | | | H | | | | |
| PLDGAV | 569 | 0.716 | 0.780 | 0.19 | PLNGAV | 1068 | 0.622 | 0.296 | 0.09 |
| HLYAQA | | | | | HLYARA | | | | |
| QTGWVQ | | | | | QTASVQ | | | | |
| H | | | | | N | | | | |
| PLNGAV | 570 | 0.823 | 0.773 | 6.31 | TLNGAV | 1069 | 0.876 | 0.295 | 0.14 |
| HLYAQA | | | | | HLYAQA | | | | |
| QTTRVQ | | | | | QRSLVQ | | | | |
| N | | | | | N | | | | |
| PLDGAV | 571 | 0.722 | 0.767 | 0.09 | PLNGAV | 1070 | 0.481 | 0.294 | 0.14 |
| HLYAQA | | | | | HLYAQA | | | | |
| QTGWLQ | | | | | HTGWVQ | | | | |
| N | | | | | K | | | | |
| PLNGSV | 572 | 0.681 | 0.757 | 8.32 | PLNGDV | 1071 | 0.859 | 0.294 | 0.14 |
| HLYAQP | | | | | HLYAQA | | | | |
| QTGWVQ | | | | | QTAQVQ | | | | |
| N | | | | | N | | | | |
| PLNGAV | 573 | 0.794 | 0.755 | 25.1 | TLNGAV | 1072 | 0.407 | 0.294 | 0.14 |
| HLYSQA | | | | | HLYAQA | | | | |
| QTGLVQ | | | | | QTQPVQ | | | | |
| N | | | | | N | | | | |
| PLNGAV | 574 | 0.654 | 0.755 | 14.16 | PLNGAV | 1073 | 0.505 | 0.294 | 0.7 |
| HLYAQA | | | | | HLYAQA | | | | |
| QTQWVQ | | | | | QTPRVQ | | | | |
| N | | | | | N | | | | |
| PLNGSV | 575 | 0.187 | 0.754 | 0.33 | PRNGAV | 1074 | 0.200 | 0.293 | 0.09 |
| HLYAQA | | | | | HLYAQA | | | | |
| QTGWVQ | | | | | QTMSVQ | | | | |
| I | | | | | N | | | | |
| PLNGAV | 576 | 0.259 | 0.753 | 0.28 | PLNGAV | 1075 | 0.118 | 0.293 | 0.23 |
| HLYAQA | | | | | HLYAQA | | | | |
| QTGSVH | | | | | HTVSVQ | | | | |
| N | | | | | N | | | | |
| ALNGAV | 577 | 0.367 | 0.751 | 1129 | PVNGAV | 1076 | 0.823 | 0.293 | 0.14 |
| HLYAQA | | | | | HLYAQA | | | | |
| QTGWVQ | | | | | QTGLVQ | | | | |
| N | | | | | T | | | | |
| PLNGTV | 578 | 0.224 | 0.749 | 0.14 | PLNGAV | 1077 | 0.899 | 0.292 | 87.25 |
| HLYAQA | | | | | HLYAQA | | | | |
| QTGWVR | | | | | QSSSVQ | | | | |
| N | | | | | N | | | | |
| PLNGAV | 579 | 0.240 | 0.749 | 0.23 | PLNGAV | 1078 | 0.497 | 0.292 | 0.84 |
| HLYAQA | | | | | HLYAQA | | | | |
| QTGGLQ | | | | | QRPPVQ | | | | |
| N | | | | | N | | | | |
| SLNGAV | 580 | 0.712 | 0.748 | 0.09 | PLNGAV | 1079 | 0.779 | 0.291 | 0.09 |
| HLYAQA | | | | | HLYTQA | | | | |
| QVSGVQ | | | | | QVSPVQ | | | | |
| N | | | | | N | | | | |
| PLNGAV | 581 | 0.404 | 0.747 | 0.37 | PLNGAV | 1080 | 0.661 | 0.291 | 0.09 |
| HLYAQA | | | | | PLYAQA | | | | |
| QTAWVQ | | | | | QTTPVQ | | | | |
| K | | | | | N | | | | |
| PLNGAV | 582 | 0.660 | 0.747 | 0.19 | ALNGAV | 1081 | 0.293 | 0.290 | 1.59 |
| HLYAQA | | | | | HLYAQA | | | | |
| QTGRVQ | | | | | QTGWVK | | | | |
| T | | | | | N | | | | |
| TLNGAV | 583 | 0.754 | 0.739 | 0.09 | SLNGAV | 1082 | 0.871 | 0.290 | 0.56 |
| HLYAQA | | | | | HLYAQA | | | | |
| QSAPVQ | | | | | QTGWVR | | | | |
| N | | | | | N | | | | |
| PLNGAV | 584 | 0.449 | 0.738 | 0.09 | PLNGAV | 1083 | 0.316 | 0.288 | 0.09 |
| HLYDQA | | | | | HLYAQA | | | | |
| QTVAVQ | | | | | QLAGVK | | | | |
| N | | | | | N | | | | |
| QLNGAV | 585 | 0.892 | 0.735 | 48.7 | PLNGAV | 1084 | 0.427 | 0.288 | 0.09 |
| HLYAQA | | | | | HLYAQA | | | | |
| QTGSVQ | | | | | QTSPAQ | | | | |
| N | | | | | N | | | | |
| PLNGAV | 586 | 0.858 | 0.733 | 5.37 | PLNGAV | 1085 | 0.539 | 0.288 | 0.19 |
| HLYAQA | | | | | HLYAKA | | | | |
| QNTQVQ | | | | | QLSSVQ | | | | |
| N | | | | | N | | | | |
| PLNGAV | 587 | 0.512 | 0.729 | 5.05 | PLNGAV | 1086 | 0.526 | 0.285 | 0.14 |
| HLYAQA | | | | | HLYAQA | | | | |
| QTGWLQ | | | | | QRSPVQ | | | | |
| N | | | | | T | | | | |
| PINGAV | 588 | 0.684 | 0.721 | 45.57 | PLNGAV | 1087 | 0.826 | 0.285 | 0.14 |
| HLYAQA | | | | | HLYAHA | | | | |
| QTGLVQ | | | | | QTMSVQ | | | | |
| N | | | | | N | | | | |
| PLDGAV | 589 | 0.600 | 0.719 | 0.09 | PLNGNV | 1088 | 0.429 | 0.284 | 0.14 |
| HLYAQA | | | | | HLYAQA | | | | |
| QTGWVQ | | | | | QTGWVK | | | | |
| I | | | | | N | | | | |
| PLNGSV | 590 | 0.369 | 0.713 | 0.09 | PLNGAV | 1089 | 0.409 | 0.284 | 0.09 |
| HLYAQA | | | | | HLYAQA | | | | |
| QTGSVQ | | | | | QTGWVP | | | | |
| T | | | | | H | | | | |
| PLNGAV | 591 | 0.460 | 0.711 | 0.09 | PLNGAV | 1090 | 0.442 | 0.284 | 0.09 |
| QLYAQA | | | | | HLYAQA | | | | |
| QTSSVQ | | | | | QPGWGQ | | | | |
| N | | | | | N | | | | |
| PLNGAV | 592 | 0.658 | 0.707 | 5.61 | PLNGVV | 1091 | 0.291 | 0.284 | 0.14 |
| HLYAQA | | | | | HLYAQA | | | | |
| QTYAVQ | | | | | QTVSVQ | | | | |
| N | | | | | N | | | | |
| PLNGAV | 593 | 0.575 | 0.703 | 81.37 | PLNGDV | 1092 | 0.499 | 0.283 | 0.19 |
| HLYAQA | | | | | HLYAQA | | | | |
| QTLSVQ | | | | | QTSSVQ | | | | |
| N | | | | | N | | | | |
| PLNGAV | 594 | 0.874 | 0.703 | 12.15 | PLNGAV | 1093 | 0.570 | 0.283 | 0.14 |
| HLYAQA | | | | | HIYAQA | | | | |
| QNLPVQ | | | | | QTLSVQ | | | | |
| N | | | | | N | | | | |
| PLNGAV | 595 | 0.328 | 0.702 | 0.09 | PLNGAV | 1094 | 0.597 | 0.283 | 0.19 |
| HLYAQA | | | | | HLYAQA | | | | |
| QTSSDQ | | | | | QIPPVQ | | | | |
| N | | | | | N | | | | |
| PLNGAV | 596 | 0.736 | 0.702 | 23.93 | SLNGAV | 1095 | 0.856 | 0.282 | 1.03 |
| HLYAQA | | | | | HLYAQA | | | | |
| QATSVQ | | | | | QTGWVQ | | | | |
| N | | | | | H | | | | |
| TLNGAV | 597 | 0.353 | 0.701 | 0.23 | PLNAAV | 1096 | 0.466 | 0.282 | 0.14 |
| HLYAQA | | | | | HLYAQA | | | | |
| QLSPVQ | | | | | QTGWDQ | | | | |
| N | | | | | N | | | | |
| PLNGAV | 598 | 0.271 | 0.700 | 0.14 | PLNGAV | 1097 | 0.823 | 0.282 | 0.09 |
| HLYAQA | | | | | HRYAQA | | | | |
| QTAWFQ | | | | | QTTSVQ | | | | |
| N | | | | | N | | | | |
| TLNGAV | 599 | 0.801 | 0.700 | 0.09 | PLNGSV | 1098 | 0.394 | 0.281 | 0.09 |
| HLYAQA | | | | | HLYAQA | | | | |
| QVTPVQ | | | | | QPGSVQ | | | | |
| N | | | | | N | | | | |
| TLNGAV | 600 | 0.202 | 0.696 | 0.09 | PLDGAV | 1099 | 0.838 | 0.281 | 0.09 |
| HLYAQA | | | | | HLYAQA | | | | |
| QTAAVQ | | | | | QTGWVE | | | | |
| N | | | | | N | | | | |
| PLNGAV | 601 | 0.801 | 0.693 | 7.76 | PLNGAV | 1100 | 0.524 | 0.281 | 0.33 |
| HLYAQA | | | | | HLYAQA | | | | |
| QATQVQ | | | | | QTGRVK | | | | |
| N | | | | | N | | | | |
| RLDGAV | 602 | 0.861 | 0.693 | 8.37 | PLNGAV | 1101 | 0.435 | 0.281 | 0.33 |
| HLYAQA | | | | | HLYAQA | | | | |
| QTGWVQ | | | | | QTGRLQ | | | | |
| N | | | | | N | | | | |
| QLNGAV | 603 | 0.840 | 0.691 | 0.09 | PLNGSV | 1102 | 0.584 | 0.281 | 0.28 |
| HLYAQA | | | | | HLYAQA | | | | |
| QTGWVE | | | | | QTARVQ | | | | |
| N | | | | | N | | | | |
| PLNGAV | 604 | 0.278 | 0.691 | 0.19 | PLNGAV | 1103 | 0.378 | 0.280 | 0.19 |
| HLYAQA | | | | | HLYDQA | | | | |
| QVSSVK | | | | | QLSSVQ | | | | |
| N | | | | | N | | | | |
| PLNGAV | 605 | 0.582 | 0.690 | 94.87 | ALNGAV | 1104 | 0.550 | 0.280 | 0.09 |
| HLYAQA | | | | | HLYAQA | | | | |
| QLSSVQ | | | | | QTRLVQ | | | | |
| N | | | | | N | | | | |
| PLNGAV | 606 | 0.209 | 0.689 | 0.33 | PLNGAV | 1105 | 0.780 | 0.280 | 0.19 |
| HLYAQA | | | | | NLYAQA | | | | |
| QTGSGQ | | | | | QTAQVQ | | | | |
| N | | | | | N | | | | |
| PLNGAV | 607 | 0.441 | 0.686 | 0.09 | PLNGAV | 1106 | 0.461 | 0.279 | 0.09 |
| HLYAQA | | | | | HLYAQA | | | | |
| QLQPVQ | | | | | QLSSVQ | | | | |
| T | | | | | H | | | | |
| PINGAV | 608 | 0.805 | 0.686 | 0.09 | PLNGAV | 1107 | 0.799 | 0.279 | 0.09 |
| HLYAQA | | | | | HLYAQA | | | | |
| QLSSVQ | | | | | HTGPVQ | | | | |
| N | | | | | N | | | | |
| PINGAV | 609 | 0.313 | 0.684 | 0.09 | PLKGAV | 1108 | 0.872 | 0.279 | 0.09 |
| HLYAQA | | | | | HLYAQA | | | | |
| QTVAVQ | | | | | QTLSVQ | | | | |
| N | | | | | N | | | | |
| PLNGAV | 610 | 0.546 | 0.680 | 0.09 | ALNGAV | 1109 | 0.499 | 0.279 | 0.09 |
| HLYAQA | | | | | HLYAQA | | | | |
| QVSAVK | | | | | QKAWVQ | | | | |
| N | | | | | N | | | | |
| SLNGAV | 611 | 0.461 | 0.680 | 0.23 | PLNGAV | 1110 | 0.386 | 0.278 | 0.09 |
| HLYAQA | | | | | HLYAQA | | | | |
| QTVAVQ | | | | | QAGWVQ | | | | |
| N | | | | | T | | | | |
| PLNGAV | 612 | 0.882 | 0.680 | 22.29 | PLNGAV | 1111 | 0.709 | 0.278 | 0.19 |
| HLYAQA | | | | | HLYAQA | | | | |
| QTQAVQ | | | | | QTGLVQ | | | | |
| N | | | | | S | | | | |
| QLNGAV | 613 | 0.398 | 0.679 | 0.28 | PLNGAV | 1112 | 0.436 | 0.277 | 0.14 |
| HLYAQA | | | | | HLYAQA | | | | |
| QTAQVQ | | | | | QTGCVQ | | | | |
| N | | | | | I | | | | |
| PLNGAV | 614 | 0.309 | 0.676 | 0.14 | QLNGAV | 1113 | 0.529 | 0.276 | 1.26 |
| HLYDQA | | | | | HLYAQA | | | | |
| QTMSVQ | | | | | QPGWVQ | | | | |
| N | | | | | N | | | | |
| PLNGAV | 615 | 0.341 | 0.675 | 0.19 | PLNGAV | 1114 | 0.678 | 0.276 | 0.37 |
| HLYAQP | | | | | HLYAQA | | | | |
| QTGWVQ | | | | | QRPGVQ | | | | |
| K | | | | | N | | | | |
| PLNGAV | 616 | 0.855 | 0.672 | 30.47 | PLNGAV | 1115 | 0.265 | 0.276 | 0.09 |
| HLYAQA | | | | | HLYAQA | | | | |
| QVMPVQ | | | | | QTAAVQ | | | | |
| N | | | | | H | | | | |
| PLNGAV | 617 | 0.208 | 0.669 | 0.19 | PLNGAD | 1116 | 0.728 | 0.276 | 0.09 |
| HLYAQA | | | | | HLYAQA | | | | |
| QTVSVQ | | | | | QTMPVQ | | | | |
| K | | | | | N | | | | |
| PLNGAV | 618 | 0.410 | 0.664 | 0.09 | PLNGAV | 1117 | 0.198 | 0.276 | 0.33 |
| HLYAQA | | | | | HLYAKA | | | | |
| QTAWAQ | | | | | QTGWVQ | | | | |
| N | | | | | T | | | | |
| PLNGAV | 619 | 0.764 | 0.664 | 12.06 | QLNGAV | 1118 | 0.819 | 0.275 | 0.09 |
| HLYAQA | | | | | HLYAQA | | | | |
| QNALVQ | | | | | QKAWVQ | | | | |
| N | | | | | N | | | | |
| TLNGAV | 620 | 0.217 | 0.663 | 0.09 | PLNGAV | 1119 | 0.116 | 0.274 | 0.98 |
| HLYAQA | | | | | HLYAQA | | | | |
| QVSAVQ | | | | | QTGCDQ | | | | |
| N | | | | | N | | | | |
| PLKGAV | 621 | 0.611 | 0.663 | 0.09 | PLNGTV | 1120 | 0.513 | 0.274 | 0.09 |
| HLYAQA | | | | | HLYAQE | | | | |
| QTTPVQ | | | | | QTGSVQ | | | | |
| N | | | | | N | | | | |
| QLNGSV | 622 | 0.776 | 0.657 | 96.83 | PLNGAV | 1121 | 0.671 | 0.274 | 0.09 |
| HLYAQA | | | | | NLYAQA | | | | |
| QTGWVQ | | | | | QSSLVQ | | | | |
| N | | | | | N | | | | |
| SLNGAV | 623 | 0.307 | 0.650 | 0.09 | ALNGAV | 1122 | 0.296 | 0.274 | 0.23 |
| HLYAQA | | | | | HLYAQA | | | | |
| QVATVQ | | | | | QTGWVE | | | | |
| N | | | | | N | | | | |
| PLNGAV | 624 | 0.876 | 0.650 | 9.39 | PLNGTV | 1123 | 0.405 | 0.274 | 0.14 |
| HLYAQA | | | | | HLYAQA | | | | |
| QRMLVQ | | | | | QTGWVQ | | | | |
| N | | | | | Y | | | | |
| PLNGAV | 625 | 0.589 | 0.649 | 0.09 | PINGAV | 1124 | 0.373 | 0.273 | 0.75 |
| HLYAQA | | | | | HLYAQA | | | | |
| QTVAVK | | | | | QTGWVQ | | | | |
| N | | | | | K | | | | |
| PLNGAV | 626 | 0.523 | 0.649 | 0.14 | PLNGAV | 1125 | 0.820 | 0.273 | 0.09 |
| HLYDQA | | | | | HLYAQA | | | | |
| QTTSVQ | | | | | QTGRVR | | | | |
| N | | | | | N | | | | |
| PLNGAV | 627 | 0.725 | 0.647 | 11.92 | PLNGAV | 1126 | 0.590 | 0.272 | 0.37 |
| HLYAQA | | | | | HLYAQA | | | | |
| QRQLVQ | | | | | QTGRDQ | | | | |
| N | | | | | N | | | | |
| PLNGAV | 628 | 0.754 | 0.645 | 28.79 | QLNGAV | 1127 | 0.409 | 0.272 | 0.23 |
| HLYAQA | | | | | HLYAQA | | | | |
| QTLGVQ | | | | | QSALVQ | | | | |
| N | | | | | N | | | | |
| PLNGAV | 629 | 0.889 | 0.645 | 0.09 | PLNGVV | 1128 | 0.422 | 0.272 | 0.09 |
| HLYAQA | | | | | HLYAQA | | | | |
| QLGPVQ | | | | | QTGWVQ | | | | |
| T | | | | | K | | | | |
| PLNGGV | 630 | 0.554 | 0.644 | 0.14 | PLNGAV | 1129 | 0.534 | 0.272 | 0.14 |
| HLYAQA | | | | | HLYAQA | | | | |
| QTGWVQ | | | | | HTGWGQ | | | | |
| D | | | | | N | | | | |
| PLNGAV | 631 | 0.205 | 0.640 | 0.98 | PLNGDV | 1130 | 0.638 | 0.272 | 0.09 |
| HLYAQA | | | | | HLYAQA | | | | |
| QTGSVK | | | | | QRSLVQ | | | | |
| N | | | | | N | | | | |
| PLNGAV | 632 | 0.332 | 0.635 | 0.09 | PLNGNV | 1131 | 0.711 | 0.272 | 0.09 |
| NLYAQA | | | | | HLYAQA | | | | |
| QLASVQ | | | | | QTVWVQ | | | | |
| N | | | | | N | | | | |
| PLNGAV | 633 | 0.307 | 0.634 | 0.14 | PLNGAV | 1132 | 0.332 | 0.272 | 0.23 |
| HLYDQA | | | | | HLYAQA | | | | |
| QVSSVQ | | | | | PTGWVQ | | | | |
| N | | | | | H | | | | |
| PLNGAV | 634 | 0.355 | 0.632 | 0.14 | PLNGAV | 1133 | 0.725 | 0.271 | 0.23 |
| HLYAQA | | | | | HLYSQA | | | | |
| QTMSDQ | | | | | QTGWGQ | | | | |
| N | | | | | N | | | | |
| PLNGAV | 635 | 0.337 | 0.630 | 17.48 | ALNGSV | 1134 | 0.729 | 0.271 | 0.09 |
| HLYAQA | | | | | HLYAQA | | | | |
| QTGWVQ | | | | | QTGWVQ | | | | |
| K | | | | | T | | | | |
| PLNGAV | 636 | 0.203 | 0.630 | 0.09 | PLNGAV | 1135 | 0.347 | 0.271 | 0.09 |
| HLYAQA | | | | | QLYAQA | | | | |
| QLASVK | | | | | QRSTVQ | | | | |
| N | | | | | N | | | | |
| PLNGGV | 637 | 0.490 | 0.628 | 0.23 | PLNGTV | 1136 | 0.434 | 0.271 | 0.28 |
| HLYAQA | | | | | HLYAQA | | | | |
| QTGWGQ | | | | | QTGTVQ | | | | |
| N | | | | | N | | | | |
| QLNGSV | 638 | 0.844 | 0.627 | 0.09 | PLNGTV | 1137 | 0.549 | 0.271 | 0.37 |
| HLYAQA | | | | | HLYAQA | | | | |
| QTGWVQ | | | | | QTGWVQ | | | | |
| T | | | | | I | | | | |

+0 TABLE 24
NGS fold-enrichment of TTD-001 matured AAV capsid variants brain
and DRG of cynomolgus macaques and the brain of marmosets

| | | Brain of | | DRG of | | | |
|---|---|---|---|---|---|---|---|
| | | cynomolgus | | cynomolgus | | Brain | |
| | | macaques | | macaques | | of marmosets | |
| | | Fold | Fold | Fold | Fold | Fold | Fold |
| | | change | change | change | change | change | change |
| | SEQ | relative | relative | relative | relative | relative | relative |
| | ID | to | to | to | to | to | to |
| Sequence | NO | AAV9 | TTD-001 | AAV9 | TTD-001 | AAV9 | TTD-001 |
| ALNGAVHLYAQAQTG | 1122 | 12.707 | 0.274 | 0.000 | 0.000 | 420.579 | 571.155 |
| WVEN | | | | | | | |
| PINGAVHLYAQAQTG | 40 | 8.716 | 0.188 | 2.100 | 13.718 | 573.779 | 779.203 |
| WVEN | | | | | | | |
| PLNGAVHLNAQAQTG | 41 | 0.919 | 0.020 | 2.175 | 14.212 | 677.878 | 920.572 |
| WVEN | | | | | | | |
| PLNGAVHLYAQAQSG | 42 | 5.505 | 0.119 | 0.000 | 0.000 | 263.098 | 357.292 |
| WVEN | | | | | | | |
| PLNGAVHLYAQAQTA | 43 | 8.721 | 0.188 | 0.827 | 5.405 | 236.026 | 320.528 |
| WVEN | | | | | | | |
| PLNGAVHLYAQAQTG | 44 | 4.126 | 0.089 | 0.245 | 1.603 | 475.164 | 645.282 |
| CVEN | | | | | | | |
| PLNGAVHLYAQAQTG | 871 | 18.851 | 0.406 | 0.491 | 3.206 | 288.900 | 392.332 |
| GVEN | | | | | | | |
| PLNGAVHLYAQAQTG | 45 | 5.050 | 0.109 | 0.000 | 0.000 | 541.666 | 735.593 |
| LVEN | | | | | | | |
| PLNGAVHLYAQAQTG | 818 | 20.878 | 0.450 | 2.856 | 18.662 | 571.096 | 775.559 |
| SVEN | | | | | | | |
| PLNGAVHLYAQAQTG | 804 | 21.437 | 0.462 | 1.362 | 8.901 | 381.679 | 518.327 |
| WVEN | | | | | | | |
| PLNGSVHLYAQAQTG | 499 | 43.595 | 0.939 | 2.334 | 15.248 | 490.137 | 665.615 |
| WVEN | | | | | | | |
| PVNGAVHLYAQAQTG | 46 | 6.311 | 0.136 | 0.000 | 0.000 | 506.376 | 687.669 |
| WVEN | | | | | | | |
| QLNGAVHLYAQAQTG | 603 | 32.086 | 0.691 | 0.000 | 0.000 | 290.854 | 394.985 |
| WVEN | | | | | | | |
| SLNGAVHLYAQAQTG | 47 | 8.707 | 0.188 | 2.111 | 13.795 | 302.059 | 410.202 |
| WVEN | | | | | | | |
| TLNGAVHLYAQAQTG | 48 | 0.707 | 0.015 | 0.624 | 4.074 | 254.330 | 345.385 |
| WVEN | | | | | | | |
| ALNGAVHLYAQAQTG | 819 | 20.878 | 0.450 | 0.667 | 4.356 | 232.682 | 315.986 |
| WVHN | | | | | | | |
| PHNGAVHLYAQAQTG | 49 | 3.604 | 0.078 | 4.410 | 28.816 | 177.855 | 241.530 |
| WVHN | | | | | | | |
| PINGAVHLYAQAQTG | 50 | 6.387 | 0.138 | 1.083 | 7.076 | 124.477 | 169.043 |
| WVHN | | | | | | | |
| PLKGAVHLYAQAQTG | 51 | 0.986 | 0.021 | 0.516 | 3.368 | 118.166 | 160.472 |
| WVHN | | | | | | | |
| PLNGAAHLYAQAQTG | 52 | 0.757 | 0.016 | 1.024 | 6.693 | 91.230 | 123.892 |
| WVHN | | | | | | | |
| PLNGADHLYAQAQTG | 53 | 0.842 | 0.018 | 0.540 | 3.529 | 15.514 | 21.069 |
| WVHN | | | | | | | |
| PLNGAGHLYAQAQTG | 54 | 1.198 | 0.026 | 1.451 | 9.479 | 106.338 | 144.409 |
| WVHN | | | | | | | |
| PLNGALHLYAQAQTG | 55 | 2.023 | 0.044 | 0.000 | 0.000 | 148.895 | 202.202 |
| WVHN | | | | | | | |
| PLNGAVDLYAQAQTG | 56 | 3.104 | 0.067 | 0.000 | 0.000 | 149.296 | 202.747 |
| WVHN | | | | | | | |
| PLNGAVHHYAQAQTG | 57 | 2.959 | 0.064 | 1.184 | 7.735 | 162.209 | 220.283 |
| WVHN | | | | | | | |
| PLNGAVHIYAQAQTG | 58 | 2.284 | 0.049 | 0.000 | 0.000 | 160.204 | 217.560 |
| WVHN | | | | | | | |
| PLNGAVHLDAQAQTG | 59 | 0.667 | 0.014 | 0.333 | 2.175 | 298.308 | 405.108 |
| WVHN | | | | | | | |
| PLNGAVHLNAQAQTG | 60 | 2.297 | 0.050 | 2.821 | 18.432 | 417.906 | 567.525 |
| WVHN | | | | | | | |
| PLNGAVHLSAQAQTG | 61 | 1.946 | 0.042 | 1.987 | 12.982 | 427.101 | 580.011 |
| WVHN | | | | | | | |
| PLNGAVHLYAKAQTG | 62 | 7.514 | 0.162 | 0.000 | 0.000 | 75.629 | 102.705 |
| WVHN | | | | | | | |
| PLNGAVHLYAQAKTG | 63 | 2.441 | 0.053 | 0.998 | 6.522 | 78.923 | 107.179 |
| WVHN | | | | | | | |
| PLNGAVHLYAQAQAG | 64 | 1.527 | 0.033 | 0.000 | 0.000 | 132.644 | 180.133 |
| WVHN | | | | | | | |
| PLNGAVHLYAQAQRG | 65 | 4.707 | 0.101 | 0.000 | 0.000 | 24.765 | 33.632 |
| WVHN | | | | | | | |
| PLNGAVHLYAQAQSG | 66 | 3.622 | 0.078 | 0.000 | 0.000 | 117.858 | 160.053 |
| WVHN | | | | | | | |
| PLNGAVHLYAQAQTA | 946 | 16.279 | 0.351 | 0.452 | 2.953 | 234.444 | 318.380 |
| WVHN | | | | | | | |
| PLNGAVHLYAQAQTG | 67 | 4.347 | 0.094 | 0.356 | 2.328 | 255.533 | 347.018 |
| CVHN | | | | | | | |
| PLNGAVHLYAQAQTG | 491 | 44.261 | 0.954 | 1.261 | 8.238 | 175.856 | 238.815 |
| GVHN | | | | | | | |
| PLNGAVHLYAQAQTG | 68 | 6.977 | 0.150 | 0.000 | 0.000 | 140.966 | 191.435 |
| LVHN | | | | | | | |
| PLNGAVHLYAQAQTG | 576 | 34.946 | 0.753 | 2.147 | 14.025 | 187.304 | 254.363 |
| SVHN | | | | | | | |
| PLNGAVHLYAQAQTG | 755 | 23.437 | 0.505 | 0.865 | 5.655 | 192.126 | 260.911 |
| WVHN | | | | | | | |
| PLNGAVHLYAQAQVS | 69 | 8.212 | 0.177 | 0.000 | 0.000 | 319.448 | 433.817 |
| AVHN | | | | | | | |
| PLNGAVHLYAQEQTG | 70 | 2.986 | 0.064 | 0.000 | 0.000 | 93.114 | 126.451 |
| WVHN | | | | | | | |
| PLNGAVHLYAQGQTG | 71 | 1.725 | 0.037 | 0.000 | 0.000 | 26.926 | 36.566 |
| WVHN | | | | | | | |
| PLNGAVHLYDQAQTG | 72 | 1.514 | 0.033 | 0.279 | 1.822 | 142.794 | 193.917 |
| WVHN | | | | | | | |
| PLNGAVHLYGQAQTG | 73 | 1.401 | 0.030 | 0.000 | 0.000 | 116.707 | 158.491 |
| WVHN | | | | | | | |
| PLNGAVHLYSQAQTG | 74 | 9.806 | 0.211 | 0.000 | 0.000 | 100.716 | 136.775 |
| WVHN | | | | | | | |
| PLNGAVHRYAQAQTG | 75 | 5.991 | 0.129 | 0.000 | 0.000 | 13.869 | 18.834 |
| WVHN | | | | | | | |
| PLNGAVHVYAQAQTG | 76 | 2.959 | 0.064 | 0.000 | 0.000 | 159.742 | 216.933 |
| WVHN | | | | | | | |
| PLNGAVNLYAQAQTG | 77 | 2.387 | 0.051 | 0.631 | 4.121 | 78.989 | 107.268 |
| WVHN | | | | | | | |
| PLNGAVPLYAQAQTG | 78 | 2.946 | 0.063 | 0.774 | 5.060 | 124.967 | 169.708 |
| WVHN | | | | | | | |
| PLNGAVQLYAQAQTG | 79 | 2.338 | 0.050 | 1.085 | 7.091 | 105.426 | 143.170 |
| WVHN | | | | | | | |
| PLNGGVHLYAQAQTG | 979 | 15.473 | 0.333 | 0.626 | 4.090 | 185.552 | 251.984 |
| WVHN | | | | | | | |
| PLNGSVHLYAQAQTG | 703 | 25.824 | 0.556 | 1.283 | 8.381 | 360.426 | 489.465 |
| WVHN | | | | | | | |
| PLNGTVHLYAQAQTG | 80 | 10.315 | 0.222 | 0.000 | 0.000 | 159.224 | 216.229 |
| WVHN | | | | | | | |
| PRNGAVHLYAQAQTG | 81 | 2.842 | 0.061 | 1.016 | 6.636 | 163.614 | 222.192 |
| WVHN | | | | | | | |
| PVNGAVHLYAQAQTG | 82 | 8.023 | 0.173 | 0.761 | 4.971 | 402.937 | 547.197 |
| WVHN | | | | | | | |
| QLNGAVHLYAQAQTG | 816 | 21.018 | 0.453 | 0.794 | 5.185 | 344.539 | 467.890 |
| WVHN | | | | | | | |
| SLNGAVHLYAQAQTG | 83 | 8.378 | 0.181 | 1.286 | 8.399 | 211.470 | 287.181 |
| WVHN | | | | | | | |
| TLNGAVHLYAQAQTG | 84 | 1.270 | 0.027 | 0.252 | 1.648 | 136.758 | 185.720 |
| WVHN | | | | | | | |
| ALNGAVHLYAQAQTG | 1081 | 13.477 | 0.290 | 0.084 | 0.546 | 9.629 | 13.077 |
| WVKN | | | | | | | |
| ALNGAVNLYAQAQTG | 85 | 4.203 | 0.091 | 0.000 | 0.000 | 3.919 | 5.323 |
| WVKN | | | | | | | |
| KLNGAVHLYAQAQTG | 86 | 7.153 | 0.154 | 0.000 | 0.000 | 9.576 | 13.004 |
| WVKN | | | | | | | |
| PHNGAVHLYAQAQTG | 87 | 1.248 | 0.027 | 0.378 | 2.467 | 5.661 | 7.688 |
| WVKN | | | | | | | |
| PINGAVHLYAQAQTG | 88 | 7.644 | 0.165 | 0.143 | 0.933 | 7.560 | 10.267 |
| WVKN | | | | | | | |
| PLKGAVHLYAQAQTG | 89 | 0.748 | 0.016 | 0.103 | 0.675 | 7.088 | 9.626 |
| WVKN | | | | | | | |
| PLNAAVHLYAQAQTG | 90 | 2.473 | 0.053 | 0.000 | 0.000 | 18.662 | 25.343 |
| WVKN | | | | | | | |
| PLNGAAHLYAQAQTG | 91 | 0.901 | 0.019 | 0.390 | 2.547 | 5.894 | 8.004 |
| WVKN | | | | | | | |
| PLNGADHLYAQAQTG | 92 | 3.270 | 0.070 | 0.316 | 2.064 | 0.970 | 1.317 |
| WVKN | | | | | | | |
| PLNGAGHLYAQAQTG | 93 | 0.378 | 0.008 | 0.209 | 1.367 | 11.306 | 15.354 |
| WVKN | | | | | | | |
| PLNGALHLYAQAQTG | 859 | 19.306 | 0.416 | 0.059 | 0.387 | 19.870 | 26.984 |
| WVKN | | | | | | | |
| PLNGAVDLYAQAQTG | 94 | 0.937 | 0.020 | 0.000 | 0.000 | 5.016 | 6.812 |
| WVKN | | | | | | | |
| PLNGAVHHYAQAQTG | 95 | 1.572 | 0.034 | 0.122 | 0.800 | 8.227 | 11.173 |
| WVKN | | | | | | | |
| PLNGAVHIYAQAQTG | 96 | 11.752 | 0.253 | 0.310 | 2.024 | 10.015 | 13.600 |
| WVKN | | | | | | | |
| PLNGAVHLDAQAQTG | 97 | 0.455 | 0.010 | 0.132 | 0.864 | 14.604 | 19.832 |
| WVKN | | | | | | | |
| PLNGAVHLNAQAQTG | 98 | 1.725 | 0.037 | 0.739 | 4.830 | 21.040 | 28.572 |
| WVKN | | | | | | | |
| PLNGAVHLSAQAQTG | 99 | 0.653 | 0.014 | 0.523 | 3.415 | 16.268 | 22.092 |
| WVKN | | | | | | | |
| PLNGAVHLYAEAQTG | 100 | 0.752 | 0.016 | 0.000 | 0.000 | 0.825 | 1.120 |
| WVKN | | | | | | | |
| PLNGAVHLYAHAQTG | 101 | 8.658 | 0.187 | 0.345 | 2.253 | 1.734 | 2.355 |
| WVKN | | | | | | | |
| PLNGAVHLYAKAQTG | 649 | 28.559 | 0.615 | 0.278 | 1.817 | 1.532 | 2.081 |
| WVKN | | | | | | | |
| PLNGAVHLYALAQTG | 102 | 2.311 | 0.050 | 0.000 | 0.000 | 9.892 | 13.433 |
| WVKN | | | | | | | |
| PLNGAVHLYAPAQTG | 103 | 3.766 | 0.081 | 0.000 | 0.000 | 5.697 | 7.737 |
| WVKN | | | | | | | |
| PLNGAVHLYAQAETG | 104 | 0.360 | 0.008 | 0.213 | 1.394 | 2.738 | 3.718 |
| WVKN | | | | | | | |
| PLNGAVHLYAQAQKG | 105 | 7.865 | 0.169 | 0.099 | 0.648 | 1.486 | 2.018 |
| WVKN | | | | | | | |
| PLNGAVHLYAQAQLS | 566 | 36.293 | 0.782 | 0.000 | 0.000 | 134.753 | 182.997 |
| PVKN | | | | | | | |
| PLNGAVHLYAQAQLS | 960 | 15.955 | 0.344 | 0.000 | 0.000 | 51.408 | 69.813 |
| SVKN | | | | | | | |
| PLNGAVHLYAQAQPG | 106 | 10.171 | 0.219 | 0.000 | 0.000 | 13.928 | 18.914 |
| WVKN | | | | | | | |
| PLNGAVHLYAQAQSG | 107 | 3.450 | 0.074 | 0.000 | 0.000 | 7.922 | 10.758 |
| WVKN | | | | | | | |
| PLNGAVHLYAQAQSS | 108 | 11.748 | 0.253 | 0.000 | 0.000 | 8.691 | 11.802 |
| LVKN | | | | | | | |
| PLNGAVHLYAQAQSS | 109 | 5.856 | 0.126 | 0.169 | 1.107 | 10.452 | 14.194 |
| SVKN | | | | | | | |
| PLNGAVHLYAQAQTA | 110 | 4.473 | 0.096 | 0.000 | 0.000 | 14.173 | 19.247 |
| CVKN | | | | | | | |
| PLNGAVHLYAQAQTA | 763 | 23.248 | 0.501 | 0.000 | 0.000 | 11.433 | 15.526 |
| SVKN | | | | | | | |
| PLNGAVHLYAQAQTA | 828 | 20.599 | 0.444 | 0.333 | 2.174 | 6.667 | 9.054 |
| WVKN | | | | | | | |
| PLNGAVHLYAQAQTE | 111 | 0.721 | 0.016 | 0.000 | 0.000 | 14.566 | 19.781 |
| WVKN | | | | | | | |
| PLNGAVHLYAQAQTG | 112 | 8.239 | 0.178 | 0.000 | 0.000 | 10.794 | 14.659 |
| AVKN | | | | | | | |
| PLNGAVHLYAQAQTG | 113 | 8.144 | 0.175 | 0.222 | 1.452 | 14.865 | 20.187 |
| CVKN | | | | | | | |
| PLNGAVHLYAQAQTG | 529 | 40.644 | 0.876 | 0.167 | 1.092 | 8.407 | 11.417 |
| GVKN | | | | | | | |
| PLNGAVHLYAQAQTG | 114 | 10.090 | 0.217 | 0.145 | 0.945 | 10.946 | 14.864 |
| LVKN | | | | | | | |
| PLNGAVHLYAQAQTG | 1100 | 13.059 | 0.281 | 0.000 | 0.000 | 2.607 | 3.541 |
| RVKN | | | | | | | |
| PLNGAVHLYAQAQTG | 631 | 29.707 | 0.640 | 0.516 | 3.369 | 7.169 | 9.735 |
| SVKN | | | | | | | |
| PLNGAVHLYAQAQTG | 765 | 23.167 | 0.499 | 0.260 | 1.700 | 10.937 | 14.852 |
| WVKN | | | | | | | |
| PLNGAVHLYAQAQTL | 720 | 25.032 | 0.539 | 1.771 | 11.569 | 10.981 | 14.913 |
| SVKN | | | | | | | |
| PLNGAVHLYAQAQTR | 1033 | 14.369 | 0.310 | 1.308 | 8.546 | 1.210 | 1.643 |
| WVKN | | | | | | | |
| PLNGAVHLYAQAQTS | 700 | 26.099 | 0.562 | 0.000 | 0.000 | 29.844 | 40.528 |
| SVKN | | | | | | | |
| PLNGAVHLYAQAQTS | 1037 | 14.347 | 0.309 | 0.000 | 0.000 | 12.484 | 16.953 |
| TVKN | | | | | | | |
| PLNGAVHLYAQAQTS | 115 | 4.212 | 0.091 | 0.000 | 0.000 | 34.651 | 47.056 |
| WVKN | | | | | | | |
| PLNGAVHLYAQAQTV | 625 | 30.131 | 0.649 | 0.000 | 0.000 | 22.666 | 30.781 |
| AVKN | | | | | | | |
| PLNGAVHLYAQAQTV | 325 | 69.266 | 1.492 | 0.231 | 1.508 | 22.335 | 30.332 |
| SVKN | | | | | | | |
| PLNGAVHLYAQAQTV | 771 | 22.784 | 0.491 | 0.000 | 0.000 | 1.612 | 2.189 |
| WVKN | | | | | | | |
| PLNGAVHLYAQAQVS | 610 | 31.581 | 0.680 | 0.354 | 2.310 | 21.595 | 29.326 |
| AVKN | | | | | | | |
| PLNGAVHLYAQAQVS | 558 | 37.158 | 0.801 | 0.000 | 0.000 | 40.525 | 55.033 |
| PVKN | | | | | | | |
| PLNGAVHLYAQEQTG | 116 | 7.694 | 0.166 | 0.000 | 0.000 | 1.548 | 2.102 |
| WVKN | | | | | | | |
| PLNGAVHLYAQSQTG | 117 | 7.014 | 0.151 | 0.000 | 0.000 | 5.931 | 8.054 |
| WVKN | | | | | | | |
| PLNGAVHLYDQAQTG | 118 | 2.761 | 0.059 | 0.146 | 0.956 | 7.745 | 10.518 |
| WVKN | | | | | | | |
| PLNGAVHLYEQAQTG | 119 | 7.658 | 0.165 | 0.000 | 0.000 | 7.124 | 9.675 |
| WVKN | | | | | | | |
| PLNGAVHLYGQAQTG | 120 | 0.874 | 0.019 | 0.000 | 0.000 | 5.745 | 7.802 |
| WVKN | | | | | | | |
| PLNGAVHLYSQAQTG | 121 | 6.910 | 0.149 | 0.000 | 0.000 | 11.682 | 15.865 |
| LVKN | | | | | | | |
| PLNGAVHLYSQAQTG | 122 | 7.536 | 0.162 | 0.245 | 1.599 | 4.415 | 5.996 |
| WVKN | | | | | | | |
| PLNGAVHLYTQAQTG | 123 | 0.595 | 0.013 | 0.166 | 1.086 | 10.157 | 13.793 |
| WVKN | | | | | | | |
| PLNGAVHPYAQAQTG | 124 | 0.788 | 0.017 | 0.178 | 1.162 | 3.527 | 4.790 |
| WVKN | | | | | | | |
| PLNGAVHRSLQAQTG | 125 | 0.153 | 0.003 | 0.449 | 2.931 | 7.938 | 10.780 |
| WVKN | | | | | | | |
| PLNGAVHRYAQAQTG | 126 | 1.878 | 0.040 | 0.000 | 0.000 | 2.446 | 3.322 |
| WVKN | | | | | | | |
| PLNGAVHVYAQAQTG | 127 | 1.937 | 0.042 | 0.123 | 0.802 | 7.871 | 10.689 |
| WVKN | | | | | | | |
| PLNGAVLLYAQAQTG | 128 | 4.149 | 0.089 | 0.211 | 1.379 | 3.878 | 5.266 |
| WVKN | | | | | | | |
| PLNGAVNLYAQAQTG | 129 | 3.505 | 0.076 | 0.000 | 0.000 | 1.145 | 1.555 |
| CVKN | | | | | | | |
| PLNGAVNLYAQAQTG | 130 | 4.586 | 0.099 | 0.262 | 1.710 | 2.124 | 2.884 |
| WVKN | | | | | | | |
| PLNGAVNLYDQAQTG | 131 | 2.486 | 0.054 | 0.000 | 0.000 | 9.468 | 12.858 |
| WVKN | | | | | | | |
| PLNGAVPLYAQAQTG | 132 | 1.622 | 0.035 | 0.000 | 0.000 | 7.002 | 9.509 |
| WVKN | | | | | | | |
| PLNGAVQLYAQAQTG | 133 | 2.014 | 0.043 | 0.381 | 2.487 | 11.888 | 16.145 |
| WVKN | | | | | | | |
| PLNGAVRSTAQAQTG | 134 | 0.505 | 0.011 | 0.662 | 4.326 | 24.950 | 33.883 |
| WVKN | | | | | | | |
| PLNGAVSLRAQAQTG | 135 | 0.725 | 0.016 | 1.267 | 8.277 | 6.291 | 8.543 |
| WVKN | | | | | | | |
| PLNGAVSSRAQAQTG | 136 | 0.739 | 0.016 | 1.091 | 7.130 | 19.478 | 26.452 |
| WVKN | | | | | | | |
| PLNGDVHLYAQAQTG | 1200 | 2.905 | 0.063 | 0.000 | 0.000 | 10.919 | 14.829 |
| CVKN | | | | | | | |
| PLNGDVHLYAQAQTG | 1201 | 2.559 | 0.055 | 0.235 | 1.539 | 9.737 | 13.224 |
| WVKN | | | | | | | |
| PLNGGVHLYAQAQTG | 864 | 19.077 | 0.411 | 0.292 | 1.905 | 4.576 | 6.214 |
| WVKN | | | | | | | |
| PLNGPVHLYAQAQTG | 1202 | 2.216 | 0.048 | 0.000 | 0.000 | 5.169 | 7.019 |
| WVKN | | | | | | | |
| PLNGSVHLYAQAQTG | 1203 | 5.707 | 0.123 | 0.000 | 0.000 | 21.281 | 28.900 |
| CVKN | | | | | | | |
| PLNGSVHLYAQAQTG | 791 | 22.050 | 0.475 | 0.184 | 1.202 | 13.110 | 17.803 |
| WVKN | | | | | | | |
| PLNGTVHLYAQAQTG | 978 | 15.482 | 0.334 | 0.000 | 0.000 | 17.576 | 23.868 |
| WVKN | | | | | | | |
| PLNSAVHLYAQAQTG | 1204 | 1.468 | 0.032 | 0.000 | 0.000 | 3.674 | 4.989 |
| WVKN | | | | | | | |
| PLSGAVHLYAQAQTG | 1205 | 0.680 | 0.015 | 0.000 | 0.000 | 8.111 | 11.015 |
| WVKN | | | | | | | |
| PPNGAVHLYAQAQTG | 1206 | 0.829 | 0.018 | 0.546 | 3.567 | 9.008 | 12.233 |
| WVKN | | | | | | | |
| PRNGAVHLYAQAQTG | 1207 | 1.194 | 0.026 | 0.421 | 2.751 | 9.143 | 12.417 |
| WVKN | | | | | | | |
| PVNGAVHLYAQAQTG | 1208 | 8.167 | 0.176 | 0.145 | 0.946 | 11.979 | 16.268 |
| WVKN | | | | | | | |
| QLNGAVHIYAQAQTG | 1209 | 6.982 | 0.150 | 0.000 | 0.000 | 14.405 | 19.563 |
| WVKN | | | | | | | |
| QLNGAVHLYAQAQTG | 1210 | 2.446 | 0.053 | 0.000 | 0.000 | 14.453 | 19.627 |
| CVKN | | | | | | | |
| QLNGAVHLYAQAQTG | 1211 | 7.518 | 0.162 | 0.000 | 0.000 | 1.613 | 2.190 |
| LVKN | | | | | | | |
| QLNGAVHLYAQAQTG | 966 | 15.815 | 0.341 | 1.130 | 7.380 | 3.272 | 4.443 |
| SVKN | | | | | | | |
| QLNGAVHLYAQAQTG | 1032 | 14.378 | 0.310 | 0.137 | 0.893 | 10.495 | 14.253 |
| WVKN | | | | | | | |
| QLNGAVHLYDQAQTG | 1212 | 2.378 | 0.051 | 0.000 | 0.000 | 1.224 | 1.663 |
| WVKN | | | | | | | |
| RLNGAVHLYAQAQTG | 1213 | 3.131 | 0.067 | 0.000 | 0.000 | 4.894 | 6.646 |
| WVKN | | | | | | | |
| SINGAVHLYAQAQTG | 1214 | 1.234 | 0.027 | 0.409 | 2.671 | 4.660 | 6.328 |
| WVKN | | | | | | | |
| SLNGAVHLYAQAQTG | 1215 | 6.104 | 0.132 | 0.328 | 2.143 | 11.122 | 15.104 |
| WVKN | | | | | | | |
| SLNGAVNLYAQAQTG | 1216 | 1.387 | 0.030 | 0.833 | 5.444 | 5.215 | 7.083 |
| WVKN | | | | | | | |
| TLNGAVHLYAQAQTG | 1217 | 2.176 | 0.047 | 0.108 | 0.707 | 5.355 | 7.272 |
| WVKN | | | | | | | |
| TLNGAVNLYAQAQTG | 1218 | 1.333 | 0.029 | 0.000 | 0.000 | 1.292 | 1.754 |
| WVKN | | | | | | | |
| PLNGAVHHYAQAQTG | 1219 | 0.937 | 0.020 | 0.000 | 0.000 | 982.466 | 1334.208 |
| WVPN | | | | | | | |
| PLNGAVHLNAQAQTG | 1220 | 3.698 | 0.080 | 3.072 | 20.072 | 5448.821 | 7399.606 |
| WVPN | | | | | | | |
| PLNGAVHLSAQAQTG | 1221 | 3.356 | 0.072 | 3.362 | 21.966 | 5527.158 | 7505.989 |
| WVPN | | | | | | | |
| PLNGAVHLYAQAQTG | 1222 | 8.959 | 0.193 | 0.969 | 6.329 | 3019.591 | 4100.664 |
| CVPN | | | | | | | |
| PLNGAVHLYAQAQTG | 314 | 71.770 | 1.546 | 2.071 | 13.534 | 2408.300 | 3270.518 |
| WVPN | | | | | | | |
| PLNGAVNLYAQAQTG | 1223 | 5.356 | 0.115 | 1.809 | 11.822 | 2103.432 | 2856.501 |
| WVPN | | | | | | | |
| PLNGTVHLYAQAQTG | 885 | 18.270 | 0.394 | 0.000 | 0.000 | 4688.758 | 6367.425 |
| WVPN | | | | | | | |
| PVNGAVHLYAQAQTG | 1016 | 14.568 | 0.314 | 0.000 | 0.000 | 1269.496 | 1724.000 |
| WVPN | | | | | | | |
| QLNGAVHLYAQAQTG | 648 | 28.572 | 0.616 | 0.000 | 0.000 | 14152.524 | 19219.405 |
| WVPN | | | | | | | |
| SLNGAVHLYAQAQTG | 1058 | 13.914 | 0.300 | 1.876 | 12.256 | 1800.081 | 2444.545 |
| WVPN | | | | | | | |
| PLNGAVHLYAQAQTG | 476 | 46.410 | 1 | 0.153 | 1 | 0.736 | 1 |
| WVQN | | | | | | | |
| AQAQAQTGWVQN | 1224 | 1 | 0.0215 | 1 | 6.533 | 1 | 1.358 |

### Example 4. Dose-Response Evaluation of TTD-001 in Non-Human Primates (NHPs)

This Example investigates the minimal dose of an AAV particle comprising a TTD-001 capsid variant (SEQ ID NO: 3623 (DNA) and 3636 (amino acid), comprising SEQ ID NO: 3648)) that is sufficient to achieve near-physiological expression of a payload, e.g., a single stranded payload, in the central nervous system of adult cynomolgus macaques (Macaca fascicularis) via intravenous systemic delivery.

AAV particles comprising the TTD-001 capsid variant comprising a single stranded viral genome encoding a hemagglutinin (HA)-tagged NHP protein under the control of a ubiquitous CBA promoter were injected intravenously into adult male NHPs (cynomolgus macaque) (n=3, 5-7 years of age) at various doses spanning a 30-fold range, which included 6.7e11 VG/kg, 2e12 VG/kg, 6.7e12 VG/kg, and 2e13 VG·kg. The in-life period was 28 days and then various CNS and peripheral tissues were collected for measuring transgene mRNA expression by RT-qPCR, viral DNA levels by ddPCR, transgene protein expression by ELISA, and biodistribution by immunohistochemistry (staining with an anti-HA antibody).

Widespread transgene expression was detected in the spinal cord and the brain the NHPs at doses of 2e12 VG/kg and above, especially in the putamen, thalamus, globus pallidus and brainstem (Tables 25-27). Viral DNA and mRNA were readily detectable in all NHPs and showed a consistent dose response (Table 25, Table 26).

More specifically, in the brain, dose-dependent distribution of the AAV particles comprising the TTD-001 capsid was observed in the cortical regions (frontal, motor, and somatosensory), caudate, putamen, thalamus, substantia nigra, globus pallidus, hippocampus, amygdala, hypothalamus, cerebellar cortex, and dentate nucleus. Additionally, for each dose administered, there was comparable distribution of the AAV particles comprising the TTD-001 capsid in each brain region, including the cortex as well as the deeper brain regions such as the caudate, putamen, thalamus, substantia nigra, globus pallidus, hippocampus, amygdala, hypothalamus, and dentate nucleus (Table 25).

With respect to the spinal cord, dose-dependent distribution of the AAV particles comprising the TTD-001 capsid was observed in the cervical, thoracic, and lumbar spinal cord regions and the relative distribution across all these regions was similar for each dosing group. As shown in Table 25, low biodistribution was measured in the DRG, but a dose-dependent distribution of the AAV particles comprising the TTD-001 capsid was observed in the cervical, thoracic, and lumbar DRG regions and the relative distribution across all DRG regions was similar for each dosing group.

With respect to the peripheral tissues, a dose-dependent distribution of the AAV particles comprising the TTD-001 capsid was observed in the liver, hear, and the vastus lateralis (muscle) (Table 25).

TABLE 25
Quantification of viral genomes (biodistribution) by ddPCR following intravenous
administration of various doses of AAV particles comprising a TTD-001 capsid

| | Quantification of viral genomes in the Brain (VG/diploid cell) | | | | | | |
|---|---|---|---|---|---|---|---|
| Dose | Frontal | Motor | Somatosensory | | | | Dentate |
| (VG/kg) | Cortex | Cortex | Cortex | Caudate | Putamen | Thalamus | Nucleus |
| 6.7e11 | 0.01 | 0.11 | 0.07 | 0.16 | 0.13 | 0.17 | 0.03 |
| 2e12 | 2.0 | 1.72 | 0.92 | 1.27 | 0.89 | 1.16 | 0.42 |
| 6.7e12 | 4.5 | 4.20 | 3.44 | 3.44 | 2.74 | 3.04 | 2.27 |
| 2e13 | 6.7 | 7.50 | 3.04 | 6.32 | 4.94 | 6.34 | 4.14 |

| Dose | Substantia | Globus | | | | Cerebellar |
|---|---|---|---|---|---|---|
| (VG/kg) | Nigra | Pallidus | Hippocampus | Amygdala | Hypothalamus | Cortex |
| 6.7e11 | 0.12 | 0.10 | 0.1 | 0.16 | 0.14 | 0.02 |
| 2e12 | 1.12 | 1.28 | 2.1 | 2.83 | 1.77 | 0.13 |
| 6.7e12 | 2.33 | 2.47 | 2.6 | 4.38 | 2.37 | 0.53 |
| 2e13 | 5.81 | 4.43 | 5.5 | 6.13 | 4.63 | 4.18 |

| | Quantification of viral genomes in the Spinal Cord and DRG (VG/diploid cell) | | | | | |
|---|---|---|---|---|---|---|
| | Cervical | Thoracic | Lumbar | | | |
| Dose | Spinal | Spinal | Spinal | Cervical | Thoracic | Lumbar |
| (VG/kg) | Cord | Cord | Cord | DRG | DRG | DRG |
| 6.7e11 | 0.06 | 0.03 | 0.05 | 0.002 | 0.003 | 0.007 |
| 2e12 | 0.74 | 0.42 | 0.64 | 0.007 | 0.006 | 0.019 |
| 6.7e12 | 1.35 | 0.94 | 1.58 | 0.015 | 0.022 | 0.033 |
| 2e13 | 5.35 | 3.48 | 6.48 | 0.060 | 0.079 | 0.070 |

| | Quantification of viral genomes in the | | | |
|---|---|---|---|---|
| | peripheral tissues (VG/diploid cell) | | | |
| Dose | | | | Vastus lateralis |
| (VG/kg) | Heart | Liver | Kidney | (muscle) |
| 6.7e11 | 0.11 | 1.2 | 0.04 | 0.002 |
| 2e12 | 0.10 | 12.4 | 0.71 | 0.02 |
| 6.7e12 | 0.30 | 68.5 | 1.18 | 0.06 |
| 2e13 | 0.56 | 132.3 | 0.84 | 0.15 |

Additionally, dose-dependent transgene mRNA expression by the AAV particles comprising the TTD-001 capsid was observed in the brain, spinal cord, DRG, and peripheral tissues (Table 26). The lowest dose of the AAV particles comprising the TTD-001 capsid protein resulted in higher transgene mRNA and protein expression than a 30-fold higher dose of wild-type AAV9. Comparison of the transgene mRNA with the matching endogenous transcript indicated that a dose of 2e12VG/kg was sufficient to achieve supra-physiological levels in the central nervous system (CNS), while showing low transduction in the liver and the dorsal root ganglia (DRG) (Table 26).

TABLE 26
Quantification of transgene mRNA by RT-qPCR following
intravenous administration of various doses of
AAV particles comprising a TTD-001 capsid
Transgene mRNA relative to housekeeping gene

| | Dose (VG/kg) | | | |
|---|---|---|---|---|
| | 6.7e11 | 2e12 | 6.7e12 | 2e13 |
| Tissue | VG/kg | VG/kg | VG/kg | VG/kg |
| Frontal Cortex | 0.02 | 0.29 | 3.42 | 4.51 |
| Motor Cortex | 0.2 | 1.65 | 9.42 | 26.5 |
| Putamen | 0.11 | 0.38 | 1.76 | 2.52 |
| Dentate Nucleus | 0.01 | 0.26 | 4.38 | 15.8 |
| Cervical Spinal Cord | 0.07 | 0.63 | 2.25 | 8.26 |
| Thoracic Spinal Cord | 0.06 | 0.72 | 2.00 | 5.37 |
| Lumbar Spinal Cord | 0.10 | 0.83 | 6.71 | 27.22 |
| Cervical DRG | 0.02 | 0.29 | 0.99 | 3.58 |
| Thoracic DRG | 0.01 | 0.28 | 1.08 | 4.36 |
| Lumbar DRG | 0.02 | 0.56 | 2.21 | 4.44 |
| Heart | 0.002 | 0.33 | 5.37 | 11.45 |
| Liver | 0.04 | 0.3 | 1.43 | 2.22 |

Transgene mRNA vs. endogenous transcript
(fold change relative to vehicle control)

| | Dose (VG/kg) | | | | |
|---|---|---|---|---|---|
| | | 6.7e11 | 2e12 | 6.7e12 | 2e13 |
| Tissue | Vehicle | VG/kg | VG/kg | VG/kg | VG/kg |
| Frontal Cortex | 1.0 | 0.97 | 2.04 | 12.79 | 17.04 |
| Motor Cortex | 1.0 | 2.3 | 9.4 | 42.0 | 118.3 |
| Putamen | 1.0 | 1.59 | 3.00 | 11.25 | 16.57 |
| Dentate Nucleus | 1.0 | 0.7 | 1.7 | 17.0 | 59.9 |
| Cervical Spinal Cord | 1.0 | 1.18 | 2.65 | 12.57 | 43.16 |
| Thoracic Spinal Cord | 1.0 | 1.07 | 2.26 | 10.61 | 27.96 |
| Lumbar Spinal Cord | 1.0 | 1.16 | 3.06 | 31.30 | 122.75 |
| Cervical DRG | 1.0 | 1.0 | 1.63 | 3.24 | 9.09 |
| Thoracic DRG | 1.0 | 0.98 | 1.4 | 3.02 | 7.72 |
| Lumbar DRG | 1.0 | 1.05 | 2.3 | 4.78 | 11.72 |
| Heart | 1.0 | 1.34 | 1.67 | 6.55 | 13.57 |
| Liver | 1.0 | 0.9 | 1.2 | 2.8 | 3.2 |

TABLE 27
Quantification of total transgene protein expression in the peripheral
tissues by ELISA following intravenous administration of various
doses of AAV particles comprising a TTD-001 capsid

| | Cervical DRG | Heart | Liver |
|---|---|---|---|
| | (Transgene | (Transgene | (Transgene |
| Dose | ng/mL relative | ng/mL relative | ng/mL relative |
| (VG/kg) | to vehicle) | to vehicle) | to vehicle) |
| Vehicle | 1.0 | 1.0 | 1.0 |
| 6.7e11 | 1.1 | 1.0 | 0.87 |
| 2e12 | 1.5 | 1.04 | 1.00 |
| 6.7e12 | 1.7 | 2.23 | 0.93 |
| 2e13 | 7.4 | 3.69 | 1.24 |

By immunohistochemistry (IHC), widespread transduction by AAV particles comprising the TTD-001 capsid variant was observed in multiple brain regions of the NHPs as compared to AAV9 at all doses administered, particularly at the medium to high doses (2e12 VG/kg, 6.7e12 VG/kg, and 2e13 VG/kg). By IHC, dose dependent expression of AAV particles comprising the TTD-001 capsid variant was observed in the brain, specifically in the temporal cortex, caudate, putamen, thalamus, substantia nigra, hippocampus, and cerebellar. Morphologically, transgene expression was observed in the neuronal cell body and the neuropil from neurons in these brain regions, including the Purkinje neurons in the cerebellar cortex and the neurons deep in the cerebellar nuclei. In the brain stem, the transgene expression was observed in various structures including the gracile-nuclei, cuneate-nuclei, and the Inferior Olivary complex.

In the spinal cord of the NHPs, dose dependent transduction was also observed in the cervical, lumbar, and thoracic regions when measured by IHC, with the most intense and widespread staining occurring at the 6.7e12 VG/kg and 2e13 VG/kg doses. Substantial staining of the motor neurons in the spinal cord was also observed at the lower dose of 2e12 VG/kg. Furthermore, the cellular tropism of the TTD-001 capsid in the spinal cord appeared to be largely neuronal and neuropil at all doses in all regions (e.g., cervical, thoracic, and lumbar) investigated.

In the DRG of the NHPs, dose dependent transduction was also observed in the cervical, lumbar, and thoracic regions, with the most staining occurring at the 6.7e12 VG/kg and 2e13 VG/kg doses. The lower dose of 2e12 VG/kg showed significantly less staining and was comparable to particles comprising an AAV9 capsid that were administered at a higher dose of 2e13 VG/kg. The cellular tropism of the TTD-001 capsid in the DRG appeared to be largely neuronal at all doses in all regions investigated.

Transduction of AAV particles comprising the TTD-001 capsid variant was also measured by IHC in various peripheral tissues of the NHPs. In the liver, the transduction observed was more variable but appeared to follow a dose-dependent trend and appeared to be lower than by particles comprising an AAV9 capsid that were administered at a dose of 2e13 VG/kg. Minimal staining was observed in the quadriceps at all doses tested. In the heart, a dose-dependent trend in transduction was also observed.

Additionally, the staining of various cells in the brain and/or spinal cord following transduction with the AAV particles comprising the TTD-001 capsid at the doses investigated was quantified. As shown in FIG. 1B, a dose of 2e13 VG/kg was sufficient to transduce >40% of total cells in highly permissive brain regions (thalamus, caudate, putamen) and >20% total cells in less permissive regions (entorhinal cortex, auditory cortex, hippocampus). Even at a lower dose of 6.7e12, this was sufficient to transduce >20% of cells in the thalamus, caudate, putamen, and cerebellum (FIG. 1A). As shown in FIG. 1C, the dose of 2e13 VG/kg also resulted in transduction of >90% SMI311-positive neurons in the thalamus, dentate and spinal cord.

Together, these data demonstrate that variant AAV capsids, including TTD-001, can achieve a large improvement of their therapeutic index by retaining strong efficacy at low dose.

### Example 5. Engineering Viral Genomes for the Expression of Anti-Tau Antibodies

A viral genomes was designed for AAV delivery of the anti-tau antibody V0022 (Table 28). The nucleotide sequence from 5′ ITR to 3′ ITR is provided below, as SEQ ID NO: 2025.

TABLE 28
V0022 sequences

| SEQ | |
|---|---|
| ID | |
| NO: | Sequence |
| 2025 | ctgcgcgctcgctcgctcactgaggccgcccgggcaaagcccgggcgtcgggcgacctttggtcgcccg |
| | gcctcagtgagcgagcgagcgcgcagagagggagtggccaactccatcactaggggttccttgtagtta |
| | atgattaacccgccatgctacttatctaccagggtaatggggatcctctagaactatagctagtcGACA |
| | TTGATTATTGACTAGTTATTAATAGTAATCAATTACGGGGTCATTAGTTCATAGCCCATATATGGAGTT |
| | CCGCGTTACATAACTTACGGTAAATGGCCCGCCTGGCTGACCGCCCAACGACCCCCGCCCATTGACGTC |
| | AATAATGACGTATGTTCCCATAGTAACGCCAATAGGGACTTTCCATTGACGTCAATGGGIGGAGTATTT |
| | ACGGTAAACTGCCCACTTGGCAGTACATCAAGTGTATCATATGCCAAGTACGCCCCCTATTGACGTCAA |
| | TGACGGTAAATGGCCCGCCTGGCATTATGCCCAGTACATGACCTTATGGGACTTTCCTACTTGGCAGTA |
| | CATCTACGTATTAGTCATCGCTATTACCATGtcgaggccacgttctgcttcactctccccatctccccc |
| | ccctccccacccccaattttgtatttatttattttttaattattttgtgcagcgatgggggcggggggg |
| | gggggcgcgcgccaggcggggcggggcggggcgaggggcggggcggggcgaggcggagaggtgcggcgg |
| | cagccaatcagagcggcgcgctccgaaagtttccttttatggcgaggcgaggcggcgggcggccctata |
| | aaaagcgaagcgcgcggcgggcgggagcaagcttcgtttagtgaaccgtcagatcgcctggagacgcca |
| | tccacgctgttttgacctccatagaagacaccgggaccgatccagcctccgcggattcgaatcccggcc |
| | gggaacggtgcattggaacgcggattccccgtgccaagagtgacgtaagtaccgcctatagagtctata |
| | ggcccacaaaaaatgctttcttcttttaatatacttttttgtttatcttatttctaatactttccctaa |
| | tctctttctttcagggcaataatgatacaatgtatcatgcctctttgcaccattctaaagaataacagt |
| | gataatttctgggttaaggcaatagcaatatttctgcatataaatatttctgcatataaattgtaactg |
| | atgtaagaggtttcatattgctaatagcagctacaatccagctaccattctgcttttattttatggttg |
| | ggataaggctggattattctgagtccaagctaggcccttttgctaatcatgttcatacctcttatcttc |
| | ctcccacagctcctgggcaacgtgctggtctgtgtgctggcccatcactttggcaaagaattgggattc |
| | gaaccggtgccgccaccatgaacttcgggctcagcttgattttccttgtccttgttttaaaaggtgtcc |
| | agtgtcaagtgcagctgcagcagcctggcaccgagctggtgaaacctggatctagcgtgaatctgagct |
| | gcaaggccagcggctttaccttcaccagatactggatgcactgggtcaaggaacggccaggccacggcc |
| | tggaatggatcggcaatatcaaccccaacaacggcggaacagatttcaacgagaagttcaagaacaagg |
| | ctacactgaccgtgcacaaaagctccaccaccgtgttcatccagctgagctctctgacaagcgaggaca |
| | gcgccgtgtactattgtgccagaggcaccggcaccggcgccatggactactggggccagggaacatctg |
| | tgacagtgtccagcgccaaaacgacacccccatctgtctatccactggcccctggatctgctgcccaaa |
| | ctaactccatggtgaccctgggatgcctggtcaagggctatttccctgagccagtgacagtgacctgga |
| | actctggatccctgtccagcggtgtgcacaccttcccagctgtcctgcagtctgacctctacactctga |
| | gcagctcagtgactgtcccctccagcacctggcccagcgagaccgtcacctgcaacgttgcccacccgg |
| | ccagcagcaccaaggtggacaagaaaattgtgcccagggattgtggttgtaagccttgcatatgtacag |
| | tcccagaagtatcatctgtcttcatcttccccccaaagcccaaggatgtgctcaccattactctgactc |
| | ctaaggtcacgtgtgttgtggtagacatcagcaaggatgatcccgaggtccagttcagctggtttgtag |
| | atgatgtggaggtgcacacagctcagacgcaaccccgggaggagcagttcaacagcactttccgctcag |
| | tcagtgaacttcccatcatgcaccaggactggctcaatggcaaggagttcaaatgcagggtcaacagtg |
| | cagctttccctgcccccatcgagaaaaccatctccaaaaccaaaggcagaccgaaggctccacaggtgt |
| | acaccattccacctcccaaggagcagatggccaaggataaagtcagtctgacctgcatgataacagact |
| | tcttccctgaagacattactgtggagtggcagtggaatgggcagccagcggagaactacaagaacactc |
| | agcccatcatggacacagatggctcttacttcgtctacagcaagctcaatgtgcagaagagcaactggg |
| | aggcaggaaatactttcacctgctctgtgttacatgagggcctgcacaaccaccatactgagaagagcc |
| | tctcccactctcctggtAgaaagaggcgagagggcagaggaagtcttctaacatgcggtgacgtggagg |
| | agaatcccggccctatgaagttgcctgttaggctgttggtgctgatgttctggattcctgcttccagca |
| | gtgatgtggtgatgacacagacccctctgagcctgcctgtgtccctcggcgaccaggccagcatcagct |
| | gtagaagcagccaatctctggtgcacaacaatggcatcacctacctgtactggtatctgcagaaacctg |
| | gccagagccccaagctgctgatctaccgggtgtccaatcggttcagcggagtgccagatagatttggcg |
| | gatctggcagcggcaccgacttcaccctgaagatctctagagtcgaggccgaggacctgggcgtgtact |
| | tctgcttccagggcacacacgtgcccagaaccttcggcggcggaacaaagctggaaatcaagcgggctg |
| | atgctgcaccaactgtatccatcttcccaccatccagtgagcagttaacatctggaggtgcctcagtcg |
| | tgtgcttcttgaacaacttctaccccaaagacatcaatgtcaagtggaagattgatggcagtgaacgac |
| | aaaatggcgtcctgaacagttggactgatcaggacagcaaagacagcacctacagcatgagcagcaccc |
| | tcacgttgaccaaggacgagtatgaacgacataacagctatacctgtgaggccactcacaagacatcaa |
| | cttcacccattgtcaagagcttcaacaggaatgagtgttaactcgaggacggggtgaactacgcctgag |
| | gatccgatctttttccctctgccaaaaattatggggacatcatgaagccccttgagcatctgacttctg |
| | gctaataaaggaaatttattttcattgcaatagtgtgttggaattttttgtgtctctcactcggcctag |
| | gtagataagtagcatgggggttaatcattaactacaaggaacccctagtgatggagttggccactccc |
| | tctctgcgcgctcgctcgctcactgaggccgggcgaccaaaggtcgcccgacgcccgggctttgcccgg |
| | gcggcctcagtgagcgagcgagcgcgcag |

Description of SEQ ID NO: 2025

| | | |
|---|---|---|
| 2026 | ITR | gtcgcccggcctcagtgagcgagcgagcgcgcagagagggagtggccaactccatcactag |
| | | gggttcct |
| 2027 | CMVie | GACATTGATTATTGACTAGTTATTAATAGTAATCAATTACGGGGTCATTAGTTC |
| | enhancer | ATAGCCCATATATGGAGTTCCGCGTTACATAACTTACGGTAAATGGCCCGCCTG |
| | | GCTGACCGCCCAACGACCCCCGCCCATTGACGTCAATAATGACGTATGTTCCC |
| | | ATAGTAACGCCAATAGGGACTTTCCATTGACGTCAATGGGTGGAGTATTTACG |
| | | GTAAACTGCCCACTTGGCAGTACATCAAGTGTATCATATGCCAAGTACGCCCC |
| | | CTATTGACGTCAATGACGGTAAATGGCCCGCCTGGCATTATGCCCAGTACATG |
| | | ACCTTATGGGACTTTCCTACTTGGCAGTACATCTACGTATTAGTCATCGCTATT |
| | | ACCATG |
| 2028 | CBA | ccacgttctgcttcactctccccatctcccccccctccccacccccaattttgtatttatttattttttaat |
| | promoter | tattttgtgcagcgatgggggcggggggggggggcgcgccaggcggggggggcggggcgaggggggggcggg |
| | | gcgaggcggagaggtgcggcggcagccaatcagagcggcgcgctccgaaagtttccttttatggcgaggcgg |
| | | cggcggcggcggccctataaaaagcgaagcgcgcggcggg |
| 2029 | intron | tcagatcgcctggagacgccatccacgctgttttgacctccatagaagacaccgggaccgatccagcctccg |
| | | cggattcgaatcccggccgggaacggtgcattggaacgcggattccccgtgccaagagtgacgtaagtaccg |
| | | cctatagagtctataggcccacaaaaaatgctttcttcttttaatatacttttttgtttatcttatttctaa |
| | | tactttccctaatctctttctttcagggcaataatgatacaatgtatcatgcctctttgcaccattctaaag |
| | | aataacagtgataatttctgggttaaggcaatagcaatatttctgcatataaatatttctgcatataaattg |
| | | taactgatgtaagaggtttcatattgctaatagcagctacaatccagctaccattctgcttttattttatgg |
| | | ttgggataaggctggattattctgagtccaagctaggcccttttgctaatcatgttcatacctcttatcttc |
| | | ctcccacagctcctgggcaacgtgctggtctgtgtgctggcccatcactttggcaaagaatt |
| 2030 | signal | atgaacttcgggctcagcttgattttccttgtccttgttttaaaaggtgtccagtgt |
| | sequence | |
| | (HC) | |
| 2031 | VH | caagtgcagctgcagcagcctggcaccgagctggtgaaacctggatctagcgtgaatctga |
| | | gctgcaaggccagcggctttaccttcaccagatactggatgcactgggtcaaggaacggcc |
| | | aggccacggcctggaatggatcggcaatatcaaccccaacaacggcggaacagatttcaac |
| | | gagaagttcaagaacaaggctacactgaccgtgcacaaaagctccaccaccgtgttcatcc |
| | | agctgagctctctgacaagcgaggacagcgccgtgtactattgtgccagaggcaccggcac |
| | | cggcgccatggactactggggccagggaacatctgtgacagtgtccagc |
| 2032 | IgG1 | GCCAAAACGACACCCCCATCTGTCTATCCACTGGCCCCTGGATCTGCTGCCCAAACTAACT |
| | heavy | CCATGGTGACCCTGGGATGCCTGGTCAAGGGCTATTTCCCTGAGCCAGTGACAGTGACCTG |
| | chain | GAACTCTGGATCCCTGTCCAGCGGTGTGCACACCTTCCCAGCTGTCCTGCAGTCTGACCTC |
| | constant | TACACTCTGAGCAGCTCAGTGACTGTCCCCTCCAGCACCTGGCCCAGCGAGACCGTCACCT |
| | region | GCAACGTTGCCCACCCGGCCAGCAGCACCAAGGIGGACAAGAAAATTGTGCCCAGGGATTG |
| | | TGGTTGTAAGCCTTGCATATGTACAGTCCCAGAAGTATCATCTGTCTTCATCTTCCCCCCA |
| | | AAGCCCAAGGATGTGCTCACCATTACTCTGACTCCTAAGGTCACGTGTGTTGTGGTAGACA |
| | | TCAGCAAGGATGATCCCGAGGTCCAGTTCAGCTGGTTTGTAGATGATGTGGAGGTGCACAC |
| | | AGCTCAGACGCAACCCCGGGAGGAGCAGTTCAACAGCACTTTCCGCTCAGTCAGTGAACTT |
| | | CCCATCATGCACCAGGACTGGCTCAATGGCAAGGAGTTCAAATGCAGGGTCAACAGTGCAG |
| | | CTTTCCCTGCCCCCATCGAGAAAACCATCTCCAAAACCAAAGGCAGACCGAAGGCTCCACA |
| | | GGTGTACACCATTCCACCTCCCAAGGAGCAGATGGCCAAGGATAAAGTCAGTCTGACCTGC |
| | | ATGATAACAGACTTCTTCCCTGAAGACATTACTGTGGAGTGGCAGTGGAATGGGCAGCCAG |
| | | CGGAGAACTACAAGAACACTCAGCCCATCATGGACACAGATGGCTCTTACTTCGTCTACAG |
| | | CAAGCTCAATGTGCAGAAGAGCAACTGGGAGGCAGGAAATACTTTCACCTGCTCTGTGTTA |
| | | CATGAGGGCCTGCACAACCACCATACTGAGAAGAGCCTCTCCCACTCTCCTGGT |
| 2033 | furin | agaaagaggcga |
| | cleavage | |
| | site | |
| 2034 | T2A | gagggcagaggaagtcttctaacatgcggtgacgtggaggagaatcccggccct |
| | linker | |
| 2035 | signal | atgaagttgcctgttaggctgttggtgctgatgttctggattcctgcttccagcagt |
| | sequence | |
| | (LC) | |
| 2036 | VL | gatgtggtgatgacacagacccctctgagcctgcctgtgtccctcggcgaccaggccagca |
| | | tcagctgtagaagcagccaatctctggtgcacaacaatggcatcacctacctgtactggta |
| | | tctgcagaaacctggccagagccccaagctgctgatctaccgggtgtccaatcggttcagc |
| | | ggagtgccagatagatttggcggatctggcagcggcaccgacttcaccctgaagatctcta |
| | | gagtcgaggccgaggacctgggcgtgtacttctgcttccagggcacacacgtgcccagaac |
| | | cttcggcggcggaacaaagctggaaatcaag |
| 2037 | IgG | CGGGCTGATGCTGCACCAACTGTATCCATCTTCCCACCATCCAGTGAGCAGTTAACATCTG |
| | kappa | GAGGTGCCTCAGTCGTGTGCTTCTTGAACAACTTCTACCCCAAAGACATCAATGTCAAGTG |
| | light | GAAGATTGATGGCAGTGAACGACAAAATGGCGTCCTGAACAGTTGGACTGATCAGGACAGC |
| | chain | AAAGACAGCACCIACAGCATGAGCAGCACCCTCACGTTGACCAAGGACGAGTATGAACGAC |
| | constant | ATAACAGCTATACCTGTGAGGCCACTCACAAGACATCAACTTCACCCATTGTCAAGAGCTT |
| | region | CAACAGGAATGAGTGT |
| 2038 | polyA | gatctttttccctctgccaaaaattatggggacatcatgaagccccttgagcatctgacttctg |
| | sequence | gctaataaaggaaatttattttcattgcaatagtgtgttggaattttttgtgtctctcactcg |
| 2039 | ITR | aggaacccctagtgatggagttggccactccctctctgcgcgctcgctcgctcactgaggc |
| | | cgggcgaccaaaggtcgcccgacgcccgggctttgcccgggggcctcagtgagcgagcga |
| | | gcgcgcag |
| 2040 | signal | MNFGLSLIFLVLVLKGVQC |
| | sequence | |
| | (HC) | |
| 2041 | VH | QVQLQQPGTELVKPGSSVNLSCKASGFTFTRYWMHWVKERPGHGLEWIGNINPNNGGTDEN |
| | | EKFKNKATLTVHKSSTTVFIQLSSLTSEDSAVYYCARGTGTGAMDYWGQGTSVTVSS |
| 2042 | IgG1 | AKTTPPSVYPLAPGSAAQTNSMVTLGCLVKGYFPEPVTVTWNSGSLSSGVHTFPAVLQSDL |
| | heavy | YTLSSSVTVPSSTWPSETVTCNVAHPASSTKVDKKIVPRDCGCKPCICTVPEVSSVFIFPP |
| | chain | KPKDVLTITLTPKVTCVVVDISKDDPEVQFSWFVDDVEVHTAQTQPREEQFNSTFRSVSEL |
| | constant | PIMHQDWLNGKEFKCRVNSAAFPAPIEKTISKTKGRPKAPQVYTIPPPKEQMAKDKVSLTC |
| | region | MITDFFPEDITVEWQWNGQPAENYKNTQPIMDTDGSYFVYSKLNVOKSNWEAGNTFTCSVL |
| | | HEGLHNHHTEKSLSHSPG |
| 2043 | signal | MKLPVRLLVLMFWIPASSS |
| | sequence | |
| | (LC) | |
| 2044 | VL | DVVMTQTPLSLPVSLGDQASISCRSSQSLVHNNGITYLYWYLQKPGQSPKLLIYRVSNRFS |
| | | GVPDRFGGSGSGTDFTLKISRVEAEDLGVYFCFQGTHVPRTFGGGTKLEIK |
| 2045 | IgG | RADAAPTVSIFPPSSEQLTSGGASVVCFLNNFYPKDINVKWKIDGSERQNGVLNSWTDQDS |
| | kappa | KDSTYSMSSTLTLTKDEYERHNSYTCEATHKTSTSPIVKSFNRNEC |
| | light | |
| | chain | |
| | constant | |
| | region | |

Description of SEQ ID NO: 2025
ITR (SEQ ID NO: 2026); CMVie enhancer (SEQ ID NO: 2027); CBA promoter (SEQ ID NO: 2028);
intron (SEQ ID NO: 2029); nucleotide sequence encoding a signal sequence (SEQ ID NO: 2030);
nucleotide sequence encoding VH (SEQ ID NO: 2031); a nucleotide sequence encoding an IgG1
heavy chain constant region (SEQ ID NO: 2032); a furin cleavage site (SEQ ID NO: 2033); a T2A
linker (SEQ ID NO: 2034); a nucleotide sequence encoding a signal sequence (SEQ ID NO: 2035); a
nucleotide sequence encoding a VL (SEQ ID NO: 2036); a nucleotide sequence encoding a IgG kappa
light chain constant region (SEQ ID NO: 2037); polyA sequence (SEQ ID NO: 2038); ITR (SEQ ID
NO: 2039)

The viral genome construct comprises a nucleotide sequence encoding an antibody that binds to tau. The nucleotide sequence was designed to encode an antibody heavy chain signal sequence (SEQ ID NO: 2040 (amino acid) and SEQ ID NO: 2030 (DNA)); a nucleotide sequence encoding a heavy chain variable region (SEQ ID NO: 2041 (amino acid) and SEQ ID NO: 2031 (DNA); a nucleotide sequence encoding a heavy chain constant region (SEQ ID NO: 2042 (amino acid) and SEQ ID NO: 2032 (DNA)); a nucleotide sequence encoding a first linker (SEQ ID NO: 2033); a nucleotide sequence encoding a second linker (SEQ ID NO: 2034); a nucleotide sequence encoding a light chain signal sequence (SEQ ID NO: 2043 (amino acid) and SEQ ID NO: 2035 (DNA)); a nucleotide sequence encoding a light chain variable region (SEQ ID NO: 2044 (amino acid) and SEQ ID NO: 2036 (DNA)); and a nucleotide sequence encoding a light chain constant region (SEQ ID NO: 2045 (amino acid) and SEQ ID NO: 2037 (DNA)). The nucleotide sequence encoding the anti-tau antibody was operably linked to a CMVie enhance (SEQ ID NO: 2027) and a CBA promoter variant (SEQ ID NO: 2028).

The viral genome construct also comprised a 5′ ITR of SEQ ID NO: 2026, an intron of SEQ ID NO: 2029, a polyA signal region of SEQ ID NO:2038, and a 3′ ITR of SEQ ID NO: 2039.

### Example 6. Inhibition of Tau Pathology by Anti-Tau Antibodies in Hippocampal P301A Seeding Model

In this Example, the AD-PHF seeded hippocampal P301S model was used for in vivo efficacy studies of anti-tau antibodies and vectorized forms thereof. To establish the hippocampal seeding model, AD brain-derived PHFs was injected into the left hippocampus of 8-weeks-old P301S mice. Efficacy of the V0022, V0009, V0023, V0024, V0052 antibodies alone (passive immunization) and the vectorized V0022 antibody were then investigated.

Briefly, for the passive immunization, the V0022, V009, V0023, V0024, and V0052 antibodies were administered intraperitoneally at 40 mg/kg two times in the week prior to the hippocampal seeding of the PHFs (2 doses), and five additional doses (40 mg/kg per dose) were administered intraperitoneally each week after seeding. For the vectorized administration, AAV particles comprising a VOY101 capsid protein (SEQ ID NO: 1), and a viral genome of SEQ ID NO: 2025 (see, e.g., Example 4), which encoded the V0022 antibody under the control of a CMVie enhancer/CBA promoter variant (VOY101_V0022) were administered by intravenous injection two weeks prior to seeding at a dose of either 1e13 vg/kg or 3e13 vg/kg. An IgG or a PBS vehicle was administered as a control. At 6 weeks post-seeding and 8-weeks post the initial dose of either the antibodies alone or the vectorized anti-tau antibody, the brains and hippocampi of each animal were isolated for AT8 ELISA to assess tau pathology.

First, the ability of injected PHFs to induce tau pathology was determined in P301S mice without the injection of anti-tau antibodies. A significant increase of AT8 immunoreactivity (IR) was detected in the PHF injected ipsilateral site but not in vehicle (PBS) injected site. In the tau seed injected mice, tau pathology was detected in the contralateral site to a lesser extent, indicating tau pathology induced by injected PHF can spread across hippocampus. Tau pathology was also detected by IHC staining with AT100 anti-tau antibody. A significant number of CA neurons on the ipsilateral site exhibited tau pathology (AT100 positives).

Table 29 provides the reduction in AT8 IR pathology in the mice following passive administration of the V0009, V0022, V0023, V0024, and V0052 antibodies normalized to the PBS vehicle control. As shown in Table 29, a significant reduction of tau pathology was observed in both the ipsilateral and contralateral hippocampus in mice passively treated with V0022, V0023, and V0024. Additionally, all antibodies investigated demonstrated a significant reduction in tau pathology in the contralateral hippocampus. Comparing the AT8 IR in the ipsilateral hippocampus versus the contralateral hippocampus also demonstrated a robust reduction of tau pathology spreading across the hippocampus in the mice treated by passive immunization of the V0022, V0023, and V0024 anti-tau antibodies.

TABLE 29
Efficacy of anti-tau antibodies administered by
passive in vivo compared to PBS vehicle control

Reduction of AT8 IR vs vehicle (PBS)

| Antibody | Ipsilateral Efficacy | Contralateral Efficacy |
|---|---|---|
| V0009 | None | 43% |
| V0022 | 74% | 71% |
| V0023 | 52% | 55% |
| V0024 | 67% | 72% |
| V0052 | none | 55% |

Table 30 provides the reduction in AT8 IR pathology normalized to an IgG control (efficacy) as well as the viral genome and/or antibody levels in the hippocampus and cerebral spinal fluid (CSF) (biodistribution) in the mice following passive administration (V0022) or vectorized administration (VOY101_V0022) of the V0022 antibody. As shown in Table 30, a dose dependent increase in viral genome or antibody levels was observed in the central nervous system and CSF following vectorized delivery of the V0022 antibody. Additionally, significant reduction in tau pathology was observed in mice treated with the high and low doses of the vectorized V0022 antibody as well as those treated with passive delivery of the V0022 antibody. In fact, passive administration of V0022 resulted in comparable levels of reduction in tau pathology when compared to vectorized delivery at both the high and low dose. These data demonstrate that V0022 administered passively or in a vectorized form (e.g., vectorized in an AAV particle) can significantly reduce tau pathology.

TABLE 30
Efficacy and biodistribution following passive and
vectorized administration of the V0022 antibody

| | | | Hippo- | CSF | Reduction |
|---|---|---|---|---|---|
| | | | campus | Ab | of AT8 |
| | | Viral | Ab | levels | pathology |
| Antibody | | genomes | level | (ng/ | normalized to |
| (Ab) | Dose | per cell | (ng/g) | mL) | IgG control |
| VOY101_{—} | 1e13 vg/kg | 8 vg/cell | 2,878 | 358 | 71% |
| V002 2 | 3e13 vg/kg | 25 vg/cell | 7,310 | 728 | 78% |
| (vectorized) | | | | | |
| V0022 | 40 mg/kg | N/A | 887 | 194 | 68.4% |
| (passive) | | | | | |

### Example 7. Inhibition of Tau Pathology by the V0022 Antibody in a P301S Intrinsic Model

In this Example, the P301S intrinsic model was used for in vivo efficacy studies of the V0022 anti-tau antibody and a vectorized form thereof.

Eight-week-old P301S mice were dosed with AAV particles comprising a VOY101 capsid protein (SEQ ID NO: 1), and a viral genome of SEQ ID NO: 2025 (see, e.g., Example 4), which encoded the V0022 antibody under the control of a CMVie enhancer/CBA promoter variant (VOY101_V0022) by intravenous administration or the V0022 antibody by intraperitoneal injection, weekly for 13 weeks at a dose of either 1e13 vg/kg or 3e13 vg/kg for VOY101_V0022 or a dose of 40 mg/kg for the V0022 antibody. Control mice were administered with an IgG control. Tau pathology was not yet developed in these mice at 8 weeks but in untreated mice, continues to progress throughout their lifespan. At 20 weeks of age (e.g., 12 weeks post initial dose of VOY101_V0022 or V0022), mice were sacrificed and the hippocampus and cortex were isolated for biochemical analyses.

Table 31 provides the reduction in AT8 IR pathology normalized to an IgG control (efficacy) as well as the viral genome and/or antibody levels in the hippocampus and CSF (biodistribution) in the mice following passive administration (V0022) or vectorized administration (VOY101_V0022) of the V0022 antibody. As shown in Table 31, a dose dependent increase in viral genome was observed in the central nervous system regions and CSF following vectorized delivery of the V0022 antibody. V0022 antibody expression was observed in the CNS regions as well as the CSF in mice treated by both the vectorized or passive delivery of the V0022 antibody. Additionally, a reduction in tau pathology was observed in mice treated with the high and low doses of the vectorized V0022 antibody as well as those treated with passive delivery of the V0022 antibody. In fact, passive administration of V0022 resulted in comparable levels of reduction in tau pathology when compared to vectorized delivery at both the high and low dose. These data demonstrate that V0022 administered passively or in a vectorized form (e.g., vectorized in an AAV particle) can significantly reduce tau pathology.

TABLE 31
Efficacy and biodistribution in the cortex and hippocampus following
passive and vectorized administration of the V0022 antibody

| | | | | | | Reduction of AT8 | |
|---|---|---|---|---|---|---|---|
| | | | Antibody Levels | | | pathology normalized to | |
| Antibody and | Viral genomes per cell | | Cortex | Hippocampus | CSF | IgG control | |
| Dose | Cortex | Hippocampus | (ng/g) | (ng/g) | (ng/mL) | Cortex | Hippocampus |
| VOY101_V00221e13 vg/kg | 10.3 | 4.7 | 2,970 | 3405 | 330 | 42% | 53% |
| VOY101_V00223e13 vg/kg | 19.2 | 8.9 | 5,451 | 6277 | 564 | 61% | 66% |
| V002240 mg/kg | N/A | N/A | 98 | 628 | 396 | 30% | 40% |

### IX. Equivalents and Scope

Those skilled in the art will recognize, or be able to ascertain using no more than routine experimentation, many equivalents to the specific embodiments in accordance with the Detailed Description provided herein. The scope of the present disclosure is not intended to be limited to the above Detailed Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

In addition, it is to be understood that any particular embodiment of the present disclosure that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the disclosure (e.g., any, composition, therapeutic or active ingredient; any method of production; any method of use; etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit of the disclosure in its broader aspects.

While the present disclosure has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the disclosure.

All publications, patent applications, patents, and other references mentioned herein are incorporated by reference in their entirety. In case of conflict, the present specification, including definitions, will control. In addition, section headings, the materials, methods, and examples are illustrative only and not intended to be limiting.

We claim:

1. An isolated, e.g., recombinant, AAV particle comprising an AAV capsid variant and a nucleic acid comprising a transgene encoding an antibody molecule which binds to human tau (e.g., human tau, e.g., SEQ ID NO: 9200), wherein the AAV capsid variant comprises an amino sequence comprising the following formula: [N1]-[N2], wherein:
(i) [N1] comprises X1, X2, X3, X4, and X5, wherein:
(a) position X1 is: P, Q, A, H, K, L, R, S, or T;
(b) position X2 is: L, I, V, H, or R;
(c) position X3 is: N, D, I, K, or Y;
(d) position X4 is: G, A, C, R, or S; and
(e) position X5 is: A, S, T, G, C, D, N, Q, V, or Y; and
(ii) [N2] comprises the amino acid sequence of VHLY (SEQ ID NO: 4680), VHIY (SEQ ID NO: 4681), VHVY (SEQ ID NO: 4682), or VHHY (SEQ ID NO: 4683); and/or a substitution, e.g., a conservative substitution, of any of the aforesaid amino acids in (i) and/or (ii);
wherein [N1]-[N2] is present from N-terminus to C-terminus, immediately subsequent to position 586, numbered according to any one of SEQ ID NO: 5, 8, 138, or 3636;
optionally, wherein the AAV capsid variant comprises:
(a) an amino acid sequence at least 95% identical to the amino acid sequence of positions 203-743, e.g., a VP3, of any one of SEQ ID NO: 5, 8, or 3636; or
(b) an amino acid sequence at least 95% identical to the amino acid sequence of positions 203-736, e.g., a VP3, of SEQ ID NO: 138.

2. The isolated AAV particle of claim 1, wherein [N1] comprises PLNGA (SEQ ID NO: 3679), SLNGA (SEQ ID NO: 4684), QLNGA (SEQ ID NO: 4685), ALNGA (SEQ ID NO: 4686), PLNGS (SEQ ID NO: 4687), PVNGA (SEQ ID NO: 4688), PLNGG (SEQ ID NO: 4689), PLNGT (SEQ ID NO: 4690), PLDGA (SEQ ID NO: 4691), QLNGS (SEQ ID NO: 4692), PLNGN (SEQ ID NO: 4693), SLDGA (SEQ ID NO: 4694), HLNGA (SEQ ID NO: 4695), ALNGT (SEQ ID NO: 4696), PINGA (SEQ ID NO: 4697), ALDGA (SEQ ID NO: 4698), PLNCA (SEQ ID NO: 4699), PLNGQ (SEQ ID NO: 4700), PLDSA (SEQ ID NO: 4701), RLDGA (SEQ ID NO: 4702), QLNGN (SEQ ID NO: 4703), PLNGY (SEQ ID NO: 4704), PLDSS (SEQ ID NO: 4705), PLNGC (SEQ ID NO: 4706), PLYGA (SEQ ID NO: 4707), TLNGA (SEQ ID NO: 4708), PVDGA (SEQ ID NO: 4709), PLKGA (SEQ ID NO: 4710), PLNGD (SEQ ID NO: 4711), KLDGA (SEQ ID NO: 4712), PHNGA (SEQ ID NO: 4713), PLNGV (SEQ ID NO: 4714), PLNAA (SEQ ID NO: 4715), QLNGY (SEQ ID NO: 4716), PLDGS (SEQ ID NO: 4717), LLNGA (SEQ ID NO: 4718), PLNRA (SEQ ID NO: 4719), PLIGA (SEQ ID NO: 4720), PRNGA (SEQ ID NO: 4721), or ALNGS (SEQ ID NO: 4722), or a substitution, e.g., a conservative substitution, of any of the aforesaid amino acid sequences.

3. The isolated AAV particle of claim 1 or 2, wherein [N1]-[N2] comprises:
(SEQ ID NO: 3648)
(i) PLNGAVHLY,
(SEQ ID NO: 4780)
ALDGAVHLY,
(SEQ ID NO: 4781)
ALNGAVHLY,
(SEQ ID NO: 4782)
PINGAVHLY,
(SEQ ID NO: 4783)
PLDGAVHLY,
(SEQ ID NO: 4784)
PLDSAVHLY,
(SEQ ID NO: 4785)
PLDSSVHLY,
(SEQ ID NO: 4786)
PLNGGVHLY,
(SEQ ID NO: 4787)
PLNGNVHLY,
(SEQ ID NO: 4788)
PLNGSVHLY,
(SEQ ID NO: 4789)
PLNGTVHLY,
(SEQ ID NO: 4790)
QLNGAVHLY,
(SEQ ID NO: 4791)
SLDGAVHLY,
(SEQ ID NO: 4792)
SLNGAVHLY,
(SEQ ID NO: 4793)
TLNGAVHLY,
(SEQ ID NO: 4794)
PLNGAVHIY,
(SEQ ID NO: 4795)
PLDGAVHVY,
or
(SEQ ID NO: 4796)
PLNGAVHHY;
(ii) an amino acid sequence comprising any portion of an amino acid sequence in (i), e.g., any 2, 3, 4, 5, 6, 7, or 8 amino acids, e.g., consecutive amino acids, thereof;
(iii) an amino acid sequence comprising at least one, two, or three but no more than four substitutions (e.g., conservative substitutions), relative to any of the amino acid sequences in (i); or
(iv) an amino acid sequence comprising at least one, two, or three but no more than four different amino acids, relative to any one of the amino acid sequences in (i).

4. The isolated AAV particle of any one of claims 1-3, wherein the AAV capsid variant further comprises [N3], wherein [N3] comprises X6, X7, X8, and X9, wherein:
(a) position X6 is: A, D, S, or T;
(b) position X7 is: Q, K, H, L, P, or R;
(c) position X8 is: A, P, E, or R; and
(d) position X9 is: Q, H, K, or P; or a substitution, e.g., a conservative substitution, of any of the aforesaid amino acids in (a)-(d).

5. The isolated AAV particle of claim 4, wherein [N3] comprises AQAQ (SEQ ID NO: 4737), SQAQ (SEQ ID NO: 4738), AQPQ (SEQ ID NO: 4739), AQSQ (SEQ ID NO: 4740), AKAQ (SEQ ID NO: 4741), AHAQ (SEQ ID NO: 4742), AQAP (SEQ ID NO: 4743), DQAQ (SEQ ID NO: 4744), APAQ (SEQ ID NO: 4745), AQAK (SEQ ID NO: 4746), AQAH (SEQ ID NO: 4747), AQEQ (SEQ ID NO: 4748), ALAQ (SEQ ID NO: 4749), ARAQ (SEQ ID NO: 4750), or TQAQ (SEQ ID NO: 4751), or a substitution, e.g., a conservative substitution, of any of the aforesaid amino acid sequences.

6. The isolated AAV particle of any one of any one of claims 1-5, wherein the AAV capsid variant further comprises [N4], wherein [N4] comprises X10, X11, and X12, wherein:
(a) position X10 is: L, T, V, R, S, A, C, I, K, M, N, P, or Q;
(b) position X11 is: S, G, A, T, M, V, Q, L, H, I, K, N, P, R, or Y; and
(c) position X12 is: P, W, S, G, A, Q, L, M, K, C, E, F, H, R, T, V, or Y; or a substitution, e.g., a conservative substitution, of any of the aforesaid amino acids in (a)-(c).

7. The isolated AAV particle of claim 6, wherein [N4] comprises:
(i) LSP, TGW, TGL, TGS, TGG, TAW, TGR, TAS, LSS, TSS, SSL, SSS, TLS, TVS, VSS, TSP, VSP, TMS, VAS, TAL, TTS, TLP, VLP, RGW, LSG, LAS, SSP, LLP, STS, TSA, TTP, SAL, LGS, VTP, VSA, IGW, TGF, LTP, TLA, LSA, TVG, TAP, TMP, TSL, VQS, SSM, SLP, VSQ, RSS, TST, VMS, TTA, TQP, LST, LAP, TVA, RLS, TGY, TSG, TAG, VMP, TSQ, TMA, VGS, TSW, TGV, TGT, TLG, LMP, VQP, TGM, SMS, SQL, IGS, RSV, TAA, STP, LSQ, TAQ, TGP, ASP, VSG, SAP, TLQ, LQP, TAT, TGQ, ATS, IGG, VAA, TSM, TVW, TAM, TGA, VAT, QSP, TQA, VQA, RSP, LAT, VAQ, LAA, RST, RTL, LGT, LMS, LGP, RTS, SQP, VLG, SVS, TMQ, SAV, LAG, SGP, TNS, RLT, TTQ, SAA, TSV, RLG, RAS, STQ, CSP, SAG, ALP, VTS, ISP, SVG, LTS, TTT, RSG, TQL, LNP, TVQ, IAS, LAQ, LSR, LSN, TTG, TSN, SMA, TKS, SVA, TQQ, VQQ, RLP, SAM, TAV, TQW, SSR, TQT, VNS, RSA, LMG, RQS, LVG, VTA, RTT, SMG, VMA, TKP, SAQ, NSP, ATP, VAG, RGS, VKP, RMS, NLP, NAL, RTP, RQL, VQG, VTG, VST, NAS, RVE, ATG, AMS, RNS, VMQ, SMQ, LQQ, TMG, LGQ, TSH, AAP, RSQ, TYS, ITP, VAK, TQM, TKA, SQQ, ISG, VSR, RTA, RML, SQM, VAN, CTP, ISS, AGP, TAK, RTG, LHP, TMT, AQP, QAP, RQP, LKS, NTT, TSK, RYS, KSS, NTP, VGG, IAA, LMA, MAP, VHP, VLS, LAN, ATQ, TNA, TAN, VSN, AAA, AVG, LTA, SAN, RAG, RQG, TLR, LSH, SAF, RAA, IQP, ILG, VNG, SVQ, LSK, TNG, RTQ, TMN, RGG, TTR, VRP, VKA, LAR, NQP, TMK, TYA, TQK, TTK, IAG, TQN, LAH, NTQ, RQQ, RAQ, TKQ, TQH, TNQ, LMQ, VNA, VQT, TQR, VGK, VKQ, IQS, LQR, TMM, VGN, RIG, SAK, RIA, VQN, NVQ, RIP, NAQ, NMQ, TPS, LTN, VTK, PGW, LPP, SPP, TPA, TGC, VPP, TPT, TPW, TPP, RPP, TPQ, TPR, TPG, VPA, VPQ, RPG, KGW, TRW, TAR, IPP, RSL, LVP, KGS, VAP, KGG, KAW, PGS, TRL, or AGW, or a substitution, e.g., a conservative substitution, of any of the aforesaid amino acid sequences;
(ii) LSP; or
(iii) TGW.

8. The isolated AAV particle of any one of claims 1-7, wherein the AAV capsid variant further comprises [N5], wherein [N5] comprises X13, X14, and X15, wherein:
(a) position X13 is: V, D, F, G, L, A, E, or I;
(b) position X14 is: K, P Q, R, H, E, or L; and
(c) position X15 is: N, T, K, H, D, Y, S, I, or P; or a substitution, e.g., a conservative substitution, of any of the aforesaid amino acids in (a)-(c).

9. The isolated AAV particle of claim 8, wherein [N5] comprises:
(i) VQN, VPN, VKN, VQT, VQK, DQN, VQH, GQN, VQI, VHN, FQN, LQN, VLN, VRN, VQS, VQY, AQN, VEN, VQD, IQN, VKK, DKN, VKT, VQP, EQN, GQT, FQK, GHN, or VPH, or a substitution, e.g., a conservative substitution, of any of the aforesaid amino acid sequences;
(ii) VKN, VPN, VEN, or VHN; or
(iii) VQN.

10. The isolated AAV particle of claim 8 or 9, wherein [N1]-[N2]-[N3]-[N4]-[N5] comprises:
(i) the amino acid sequence of any of SEQ ID NOs: 139-1138;
(ii) the amino acid sequence of PLNGAVHLYAQAQLSPVKN (SEQ ID NO: 566);
(iii) the amino acid sequence of PLNGAVHLYAQAQTGWVPN (SEQ ID NO: 314);
(iv) the amino acid sequence of PLNGAVHLY (SEQ ID NO: 3648);
(v) the amino acid sequence of any of SEQ ID NOs: 1200-1203, 40-136, 314, 325, 491, 499, 529, 558, 566, 576, 603, 610, 625, 631, 648, 649, 700, 703, 720, 755, 763, 765, 771, 791, 804, 816, 818, 819, 828, 859, 864, 871, 885, 946, 960, 966, 978, 979, 1016, 1033, 1032, 1037, 1058, 1081, 1100, 1122, or 1204-1223;
(vi) an amino acid sequence comprising any portion of an amino acid sequence in (i)-(v), e.g., any 2, 3, 4, 5, 6, 7, 8, 9, 10, 11, 12, 13, 14, 15, 16, 17, or 18 amino acids, e.g., consecutive amino acids, thereof;
(vii) an amino acid sequence comprising at least one, two, or three but no more than four substitutions (e.g., conservative substitutions), insertions, or deletions, relative to any of the amino acid sequences in (i)-(v); or
(viii) an amino acid sequence comprising at least one, two, or three but no more than four different amino acids, relative to any one of the amino acid sequences in (i)-(v).

11. The isolated AAV particle of any one of claims 8-10, wherein the AAV capsid variant comprises from N-terminus to C-terminus [N1]-[N2]-[N3]-[N4]-[N5], wherein:
(i) [N1] is present immediately subsequent to position 586, and replaces positions 587 and 588 (e.g., A587 and Q588), numbered according to SEQ ID NO: 138;
(ii) [N2] is present immediately subsequent to [N1];
(iii) [N3] is present immediately subsequent to position 588, and replaces positions 589-592 (e.g., A589, Q590, A591, Q592), numbered according to the amino acid sequence of SEQ ID NO: 138;
(iv) [N4] is present immediately subsequent to position 592, and replaces positions 593-595 (e.g., T593, G594, W595), numbered according to the amino acid sequence of SEQ ID NO: 138;
(v) [N5] is present immediately subsequent to position 595, and replaces positions 596-598 (e.g., V596, Q597, N598), numbered according to the amino acid sequence of SEQ ID NO: 138; and/or
(vi) [N1]-[N2]-[N3]-[N4]-[N5] is present immediately subsequent to position 586 and replaces positions 587-598 (e.g., A587, Q588, A589, Q590, A591, Q592, T593, G594, W595, V596, Q597, N598), numbered according to SEQ ID NO: 138.

12. The isolated AAV particle of any one of claims 8-11, wherein:
(i) the AAV capsid variant comprises an amino acid other than A at position 587 and/or an amino acid other than Q at position 588, numbered according to SEQ ID NO: 138;
(ii) [N1] corresponds to positions 587-591 of SEQ ID NO: 5, 8, or 3636;
(iii) [N2] corresponds to positions 592 to 595 of SEQ ID NO: 5, 8, or 3636;
(iv) [N3] corresponds to positions 596-599 of SEQ ID NO: 5, 8, or 3636;
(v) [N4] corresponds to positions 600-602 of SEQ ID NO: 5, 8, or 3636;
(vi) [N5] corresponds to positions 603-605 of SEQ ID NO: 5, 8, or 3636; and/or
(vii) [N1]-[N2]-[N3]-[N4]-[N5] corresponds to positions 587-605 of SEQ ID NO: 5, 8, or 3636.

13. An isolated, e.g., recombinant, AAV particle comprising an AAV capsid variant and a nucleic acid comprising a transgene encoding an antibody molecule which binds to tau (e.g., human tau), wherein the AAV capsid variant comprises [A][B], wherein [A] comprises the amino acid sequence of PLNGA, and [B] comprises X1, X2, X3, X4, wherein:
(i) X1 is: V, I, L, A, F, D, or G;
(ii) X2 is: H, N, Q, P, D, L, R, or Y;
(iii) X3 is: L, H, I, R, or V; and
(iv) X4 is Y; and/or
wherein the AAV capsid variant comprises an amino acid modification, e.g., a conservative substitution, of any of the aforesaid amino acids in (i)-(iv);
wherein [A][B] is present from N-terminus to C-terminus, immediately subsequent to position 586, numbered according to any one of SEQ ID NO: 5, 8, 138, or 3636;
optionally wherein the AAV capsid variant comprises:
(a) an amino acid sequence at least 95% identical to the amino acid sequence of positions 203-743, e.g., a VP3, of any one of SEQ ID NO: 5, 8, or 3636; or
(b) an amino acid sequence at least 95% identical to the amino acid sequence of positions 203-736, e.g., a VP3, of SEQ ID NO: 138.

14. The isolated AAV particle of claim 13, wherein [B] is or comprises:
(i) VHLY (SEQ ID NO: 4680), VHHY (SEQ ID NO: 4683), VHIY (SEQ ID NO: 4681), VNLY (SEQ ID NO: 4724), VQLY (SEQ ID NO: 4729), IHLY (SEQ ID NO: 4730), LHLY (SEQ ID NO: 4727), VPLY (SEQ ID NO: 4723), VDLY (SEQ ID NO: 4731), AHLY (SEQ ID NO: 4732), VHRY (SEQ ID NO: 4725), FHLY (SEQ ID NO: 4726), DHLY (SEQ ID NO: 4728), VLLY (SEQ ID NO: 4733), GHLY (SEQ ID NO: 4734), VRLY (SEQ ID NO: 4735), VHVY (SEQ ID NO: 4682), or VYLY (SEQ ID NO: 4736), or a substitution, e.g., a conservative substitution, of any of the aforesaid amino acid sequences; or
(ii) VHLY (SEQ ID NO: 4680).

15. The isolated AAV particle of claim 13 or 14, wherein [A][B] comprises:
(SEQ ID NO: 3648)
(i) PLNGAVHLY,
(SEQ ID NO: 4796)
PLNGAVHHY,
(SEQ ID NO: 4794)
PLNGAVHIY,
(SEQ ID NO: 5123)
PLNGAVNLY,
(SEQ ID NO: 5124)
PLNGAVQLY,
(SEQ ID NO: 5125)
PLNGAIHLY,
(SEQ ID NO: 5126)
PLNGALHLY,
(SEQ ID NO: 5127)
PLNGAVPLY,
(SEQ ID NO: 5128)
PLNGAVDLY,
(SEQ ID NO: 5129)
PLNGAAHLY,
(SEQ ID NO: 5130)
PLNGAVHRY,
(SEQ ID NO: 5131)
PLNGAFHLY,
(SEQ ID NO: 5132)
PLNGADHLY,
(SEQ ID NO: 5133)
PLNGAVLLY,
(SEQ ID NO: 5134)
PLNGAGHLY,
(SEQ ID NO: 5135)
PLNGAVRLY,
(SEQ ID NO: 5136)
PLNGAVHVY,
or
(SEQ ID NO: 5137)
PLNGAVYLY;
(SEQ ID NO: 3648)
(ii) PLNGAVHLY;
(iii) an amino acid sequence comprising any portion of an amino acid sequence in (i) or (ii), e.g., any 2, 3, 4, 5, 6, 7, or 8 amino acids, e.g., consecutive amino acids, thereof;
(iv) an amino acid sequence comprising at least one, two, or three but no more than four substitutions (e.g., conservative substitutions), insertions, or deletions, relative to any of the amino acid sequences in (i) or (ii); or
(v) an amino acid sequence comprising at least one, two, or three but no more than four different amino acids, relative to any one of the amino acid sequences in (i) or (ii).

16. The isolated AAV particle of any one of claims 13-15, wherein the AAV capsid variant further comprises [C], wherein [C] comprises X4, X5, X6, and X7, wherein:
(a) position X4 is: A, D, S, or T;
(b) position X5 is: Q, K, H, L, P, or R;
(c) position X6 is: A, P, or E; and
(d) position X7 is: Q, H, K, or P;
or a substitution, e.g., a conservative substitution, of any of the aforesaid amino acids in (a)-(d).

17. The isolated AAV particle of claim 16, wherein [C] is or comprises:
(i) AQAQ (SEQ ID NO: 4737), AQPQ (SEQ ID NO: 4739), AKAQ (SEQ ID NO: 4741), DQAQ (SEQ ID NO: 4744), SQAQ (SEQ ID NO: 4738), AHAQ (SEQ ID NO: 4742), AQEQ (SEQ ID NO: 4748), AQAK (SEQ ID NO: 4746), ALAQ (SEQ ID NO: 4749), APAQ (SEQ ID NO: 4745), ARAQ (SEQ ID NO: 4750), AQAH (SEQ ID NO: 4747), AQAP (SEQ ID NO: 4743), or TQAQ (SEQ ID NO: 4751), or a substitution, e.g., a conservative substitution, of any of the aforesaid amino acid sequences; or
(ii) AQAQ (SEQ ID NO: 4737).

18. The isolated AAV particle of any one of claims 1-17, wherein the AAV capsid variant further comprises:
(i) the amino acid L at position 593 numbered according to SEQ ID NO: 138 or at position 600 numbered according to SEQ ID NO: 5, 8, or 3636; the amino acid S at position 594 numbered according to SEQ ID NO: 138, or at position 601 numbered according to SEQ ID NO: 5, 8, or 3636; and the amino acid P at position 595 numbered according to SEQ ID NO: 138 or at position 602 numbered according to SEQ ID NO: 5, 8, or 3636; or
(ii) the amino acid T at position 593 numbered according to SEQ ID NO: 138 or at position 600 numbered according to SEQ ID NO: 5, 8, or 3636; the amino acid G at position 594 numbered according to SEQ ID NO: 138, or at position 601 numbered according to SEQ ID NO: 5, 8, or 3636; and the amino acid W at position 595 numbered according to SEQ ID NO: 138 or at position 602 numbered according to SEQ ID NO: 5, 8, or 3636.

19. The isolated AAV particle of any one of claims 13-18, wherein the AAV capsid variant further comprises [D], wherein [D] comprises X8, X9, and X10, wherein:
(a) position X8 is: L, T, V, S, R, I, A, N, C, Q, M, P, or K;
(b) position X9 is: S, G, T, M, A, G, K, Q, V, I, R, N, P, L, H, or Y; and
(c) position X10 is: P, W, K, Q, S, C, A, G, N, T, R, V, M, H, L, E, F, or Y; or a substitution, e.g., a conservative substitution, of any of the aforesaid amino acids in (a)-(c).

20. The isolated AAV particle of any one of claims 13-19, wherein [D] is or comprises:
(i) LSP, TGW, TTK, TMK, VAQ, TAW, TGS, VKQ, SAP, LSK, LAP, LAQ, TAK, SAK, TGC, TQK, TVA, TTQ, TAQ, RIA, RAS, TTP, LTP, STP, TSP, TMQ, TSK, VSQ, VSP, TVQ, VTA, RQP, ISG, VRP, LGP, TNQ, VQQ, VAN, AAP, RST, TMA, IQP, IAS, TVS, RGS, NSP, LQP, VTG, VMQ, SMA, VGK, IQS, CSP, LQR, TPP, VTK, SSP, AGP, LAR, TTT, TGG, TLQ, TMS, VAK, RAA, TVG, LNP, LSQ, TKP, TNA, LAT, VTP, VQA, TTS, CTP, TAG, TSQ, TMN, TST, VKP, ASP, VAA, LKS, IAA, TAA, TKA, VSN, TAP, LMP, LHP, RAQ, LTN, RTT, TSV, RMS, VGN, LMQ, TAT, VHP, ISS, VAS, TRW, TMT, RSS, RTG, VAT, VTS, VSS, TNS, VKA, SGP, TGP, TAM, TQP, TQQ, VSR, VSA, VLS, TQH, LAS, QAP, NAQ, ATP, VQP, TTA, LAA, RSG, LMA, TMP, LAN, VST, SAQ, NTP, TGL, TAV, RLG, RTL, TQM, ITP, TVW, RSA, TAS, TMG, VQS, ISP, VGG, TAL, LAG, RTA, RSP, TLA, LAH, TSL, RLS, LMG, SMQ, TQT, VGS, VSG, VMA, IGG, IAG, TGR, LSH, VQT, RNS, TLP, TKQ, LGQ, NMQ, NVQ, RGG, VMS, TTG, LSR, MAP, ILG, TGT, TSS, TSH, RIG, SAM, TSM, SMG, SMS, TSG, TGA, VNS, VAG, IGS, LGS, VNG, LTA, VQN, TKS, SVG, NAS, TSA, TAN, LTS, RSQ, RIP, RVE, VLP, SVA, LQQ, LST, SAA, RTS, TQN, VNA, LMS, TMM, RSV, TQL, RTP, RQQ, VQG, PGW, STQ, QSP, RYS, TQR, SAG, RQS, SQP, STS, VLG, NQP, LGT, RAG, TGM, LSN, RLP, RQG, RLT, TLR, SAF, SVQ, LLP, RTQ, LPP, AQP, TPQ, TSW, NTT, TTR, TQW, NTQ, TYA, TLS, NLP, ATS, ATQ, LSS, TQA, VMP, NAL, RML, RQL, TLG, TGF, SAL, SQL, LSA, TGQ, TNG, AAA, SAV, LSG, SSR, SPP, LVG, TPA, KGW, VPP, ATG, SAN, SQQ, SSM, AVG, VAP, TPS, RGW, SSL, TYS, TPT, IGW, KSS, TGY, RSL, SVS, TSN, SQM, VPA, AMS, TPG, TGV, VPQ, SLP, ALP, TPW, TPR, SSS, RPP, IPP, AGW, or RPG, or a substitution, e.g., a conservative substitution, of any of the aforesaid amino acid sequences;
(ii) LSP; or
(iii) TGW.

21. The isolated AAV particle of any one of claims 1-20, wherein the AAV capsid variant further comprises:
(i) the amino acid V at position 596 numbered according to SEQ ID NO: 138 or at position 603 numbered according to SEQ ID NO: 5, 8, or 3636; the amino acid K, P, E or H at position 597 numbered according to SEQ ID NO: 138 or at position 604 numbered according to SEQ ID NO: 5, 8, or 3636; and the amino acid N at position 598 numbered according to the amino acid sequence of SEQ ID NO: 138 or at position 605 numbered according to SEQ ID NO: 5, 8, or 3636;
(ii) the amino acid V at position 596 numbered according to SEQ ID NO: 138 or at position 603 numbered according to SEQ ID NO: 5, 8, or 3636; the amino acid K at position 597 numbered according to SEQ ID NO: 138 or at position 604 numbered according to SEQ ID NO: 5, 8, or 3636; and the amino acid N at position 598 numbered according to the amino acid sequence of SEQ ID NO: 138 or at position 605 numbered according to SEQ ID NO: 5, 8, or 3636;
(iii) the amino acid V at position 596 numbered according to SEQ ID NO: 138 or at position 603 numbered according to SEQ ID NO: 5, 8, or 3636; the amino acid P at position 597 numbered according to SEQ ID NO: 138 or at position 604 numbered according to SEQ ID NO: 5, 8, or 3636; and the amino acid N at position 598 numbered according to the amino acid sequence of SEQ ID NO: 138 or at position 605 numbered according to SEQ ID NO: 5, 8, or 3636; or
(iv) the amino acid V at position 596 numbered according to SEQ ID NO: 138 or at position 603 numbered according to SEQ ID NO: 5, 8, or 3636; the amino acid Q at position 597 numbered according to SEQ ID NO: 138 or at position 604 numbered according to SEQ ID NO: 5, 8, or 3636; and the amino acid N at position 598 numbered according to the amino acid sequence of SEQ ID NO: 138 or at position 605 numbered according to SEQ ID NO: 5, 8, or 3636.

22. The isolated AAV particle of any one of claims 13-21, wherein the AAV capsid variant further comprises [E], wherein [E] comprises X11, X12, and X13, wherein:
(a) position X11 is: V, D, F, A, E, L, G, or I;
(b) position X12 is: Q, R, P, K, L, H, or E; and
(c) position X13 is: N, H, S, T, P, K, I, D, or Y; or a substitution, e.g., a conservative substitution, of any of the aforesaid amino acids in (a)-(c).

23. The isolated AAV particle of claim 22, wherein [E] comprises:
(i) VQN, DQN, VQH, FQN, VQS, VQT, VQP, VRN, VPN, VKN, AQN, VQK, EQN, VQI, LQN, GQT, VLN, VQD, VHN, GQN, VKT, VKK, FQK, VEN, VQY, DKN, GHN, IQN, or VPH, or a substitution, e.g., a conservative substitution, of any of the aforesaid amino acid sequences; or
(ii) VKN, VPN, or VQN.

24. The isolated AAV particle of claim 22 or 23, wherein [A][B][C][D][E] comprises:
(i) the amino acid sequence of any of SEQ ID NOs: 143, 148, 149, 151, 153, 154-158, 160-163, 166, 168, 170, 171, 173-175, 177-179, 181, 182, 184-188, 191-197, 199-210,212-215, 217-225, 227-231, 233, 234, 236-240, 243-262, 265, 267, 268, 270-277, 279, 282, 284-286, 288-293, 295, 296, 298, 300-314, 316-327, 329, 331, 332, 334, 336, 337-344, 346-350, 352-354, 356-365, 367, 369, 371-380, 382-385, 387, 392-394, 396, 397, 399-401, 404-411, 413-415, 417, 419-429, 432, 433, 435-437, 438, 440-442, 444-447, 450-454, 456, 458-461, 464, 465, 467-469, 471-484, 487-495, 497, 498, 500-503, 505, 507-512, 514-517, 522-525, 528-539, 542-545, 547, 551-555, 558-561, 563-568, 570, 573, 574, 576, 579, 581, 582, 584, 586, 587, 591-596, 598, 601, 604, 605, 606, 607, 610, 612, 614-619, 624-629, 631-636, 640, 641, 645, 646, 649, 650, 656, 658, 661, 663, 664, 666, 668, 669, 670, 672, 673, 674, 675, 677, 679, 683, 684, 686, 688, 689, 691, 693, 695, 696, 697, 699, 700, 701, 702, 704-706, 709-714, 720, 722, 725-731, 733, 736, 740, 745, 749-752, 754, 755, 757, 758, 760-765, 767, 768, 770, 771, 773, 778-780, 783-788, 792-794, 797-799, 801, 802, 804-806, 812, 814, 815, 817, 818, 820, 821, 824, 828, 831, 832, 834-837, 839, 840-845, 847, 848, 850-855, 857-859, 861, 862, 865, 866, 869-872, 874-876, 882-884, 887, 889-895, 897, 899, 901, 903-905, 907, 908, 910, 911, 913, 915, 919, 920, 923, 924, 926, 927, 929, 931-933, 935, 937, 939-949, 952-955, 957, 958, 960, 962, 964, 965, 967, 971, 973, 974, 976, 977, 981, 985-989, 992, 994, 997-1000, 1002, 1004, 1006-1008, 1010, 1013, 1015, 1017, 1018, 1020, 1021, 1023-1025, 1027, 1029-1031, 1033-1035, 1037-1040, 1043, 1046, 1049, 1052, 1053, 1056, 1057, 1059, 1062, 1064, 1065, 1067, 1068, 1070, 1073, 1075, 1077-1080, 1083-1087, 1089, 1090, 1093, 1094, 1097, 1100, 1101, 1103, 1105-1107, 1110-1112, 1114-1117, 1119, 1121, 1125, 1126, 1129, 1132, 1133, 1135;
(SEQ ID NO: 566)
(ii) PLNGAVHLYAQAQLSPVKN;
(SEQ ID NO: 314)
(iii) PLNGAVHLYAQAQTGWVPN;
(SEQ ID NO: 3648)
(iv) PLNGAVHLY;
(v) an amino acid sequence comprising any portion of an amino acid sequence in (i)-(iv), e.g., any 2, 3, 4, 5, 6, 7, 8, 9, 10, 11, 12, 13, 14, 15, 16, 17, or 18 amino acids, e.g., consecutive amino acids, thereof;
(vi) an amino acid sequence comprising at least one, two, or three but no more than four substitutions (e.g., conservative substitutions), insertions, or deletions, relative to any of the amino acid sequences in (i)-(iv); or
(vii) an amino acid sequence comprising at least one, two, or three but no more than four different amino acids, relative to any one of the amino acid sequences in (i)-(iv).

25. The isolated AAV particle of any one of claims 22-24, which comprises from N-terminus to C-terminus [A][B][C][D][E], wherein:
(i) [A] is present immediately subsequent to position 586, and replaces positions 587 and 588 (e.g., A587 and Q588), numbered according to SEQ ID NO: 138;
(ii) [B] is present immediately subsequent to [A];
(iii) [C] is present immediately subsequent to position 588, and replaces positions 589-592 (e.g., A589, Q590, A591, Q592), numbered according to the amino acid sequence of SEQ ID NO: 138;
(iv) [D] is present immediately subsequent to position 592, and replaces positions 593-595 (e.g., T593, G594, W595), numbered according to the amino acid sequence of SEQ ID NO: 138;
(v) [E] is present immediately subsequent to position 595, and replaces positions 596-598 (e.g., V596, Q597, N598), numbered according to the amino acid sequence of SEQ ID NO: 138; and/or
(vi) [A][B][C][D][E] is present immediately subsequent to position 586 and replaces positions 587-598 (e.g., A587, Q588, A589, Q590, A591, Q592, T593, G594, W595, V596, Q597, N598), numbered according to SEQ ID NO: 138.

26. The isolated AAV particle of any one of claims 22-25, wherein:
(i) the AAV capsid variant comprises an amino acid other than A at position 587 and/or an amino acid other than Q at position 588, numbered according to SEQ ID NO: 138;
(ii) [A] corresponds to positions 587-591 of SEQ ID NO: 5, 8, or 3636;
(iii) [B] corresponds to positions 592 to 595 of SEQ ID NO: 5, 8, or 3636;
(iv) [C] corresponds to positions 596-599 of SEQ ID NO: 5, 8, or 3636;
(v) [D] corresponds to positions 600-602 of SEQ ID NO: 5, 8, or 3636;
(vi) [E] corresponds to positions 603-605 of SEQ ID NO: 5, 8, or 3636; and/or
(vii) [A][B][C][D][E] corresponds to positions 587-605 of SEQ ID NO: 5, 8, or 3636.

27. The isolated AAV particle of any one of claims 1-26, which comprises:
(i) the amino acid sequence corresponding to positions 203-743, e.g., a VP3, of any one of SEQ ID NOs: 5, 8, or 3636, or a sequence with at least 90%, 95%, 96%, 97%, 98%, or 99% sequence identity thereto;
(ii) the amino acid sequence corresponding to positions 138-743, e.g., a VP2, of any one of SEQ ID NOs: 5, 8, or 3636, or a sequence with at least 90%, 95%, 96%, 97%, 98%, or 99% sequence identity thereto;
(iii) the amino acid sequence of any one of SEQ ID NOs: 5, 8, or 3636 (e.g., a VP1), or an amino acid sequence with at least 90%, 95%, 96%, 97%, 98%, or 99% sequence identity thereto;
(iv) an amino acid sequence comprising at least one, two or three modifications, e.g., substitutions (e.g., conservative substitutions), but not more than 30, 20 or 10 modifications, e.g., substitutions (e.g., conservative substitutions), relative to the amino acid sequence of any one of SEQ ID NO: 5, 8, or 3636; and/or
(v) an amino acid sequence comprising at least one, two or three, but not more than 30, 20 or 10 different amino acids, relative to the amino acid sequence of any one of SEQ ID NO: 5, 8, or 3636.

28. An isolated, e.g., recombinant, AAV particle comprising an AAV capsid variant and a nucleic acid comprising a transgene encoding an antibody molecule which binds to tau (e.g., human tau), wherein the AAV capsid variant comprises:
(i) the amino acid sequence of PLNGAVHLY (SEQ ID NO: 3648);
(ii) an amino acid sequence comprising at least 5, 6, 7, 8, or 9 consecutive amino acids from the amino acid sequence of PLNGAVHLY (SEQ ID NO: 3648);
(iii) an amino acid sequence comprising at least one, two, or three but no more than four substitutions (e.g., conservative substitutions), relative to the amino acid sequence of PLNGAVHLY (SEQ ID NO: 3648); or
(iv) an amino acid sequence comprising at least one, two, or three but no more than four different amino acids relative to the amino acid sequence of PLNGAVHLY (SEQ ID NO: 3648)
wherein the amino acid sequence of (i)-(iv) is present immediately subsequent to position 586, numbered according to any one of SEQ ID NOs: 5, 8, 138, or 3636;
optionally wherein the AAV capsid variant further comprises:
(a) a VP1 protein comprising the amino acid sequence of SEQ ID NO: 5, 8, 3636;
(b) a VP2 protein comprising the amino acid sequence of positions 138-743 of SEQ ID NO: 5, 8, or 3636;
(c) a VP3 protein comprising the amino acid sequence of positions 203-743 of SEQ ID NO: 5, 8, or 3636; or
(d) an amino acid sequence at least 90% (e.g., at least 92%, 95%, 96%, 97%, 98%, or 99%) identical to any one the amino acid sequences of (a)-(b).

29. The isolated AAV particle of any one of claims 1-28, wherein the antibody molecule:
(i) binds to the N-terminal region or an epitope within the N-terminal region of a tau protein (e.g., human tau),
(ii) binds to the mid-domain region or an epitope within the mid-domain region of a tau protein (e.g., human tau),
(iii) binds to the C-terminal region or an epitope within the C-terminal region of a tau protein (e.g., human tau),
(iv) binds to the microtubule-binding domain or an epitope within the microtubule-binding domain of a tau protein (e.g., human tau),
(v) binds to a region that overlaps with the N-terminal domain and the mid-domain of a tau protein (e.g., human tau),
(vi) binds to a region that overlaps with the mid-domain and the microtubule-binding domain of a tau protein (e.g., human tau), or
(vii) binds to a region that overlaps with the mid-domain and the C-terminal domain of a tau protein (e.g., human tau).

30. The isolated AAV particle of any one of claims 1-29, wherein the antibody molecule comprises a heavy chain variable region (VH) comprising an HC CDR1, an HC CDR2, and an HC CDR3, and a light chain variable region (VL) comprising an LC CDR1, an LC CDR2, and an LC CDR3, wherein:
(i) the HCDR1, HCDR2, and HCDR3 comprise the amino acid sequences of SEQ ID NOs: 1553, 1554, and 1552, respectively; and the LCDR1, LCDR2, and LCDR3 comprise the amino acid sequences of SEQ ID NOs: 1567, 1565, and 1566, respectively;
(ii) the HCDR1, HCDR2, and HCDR3 comprise the amino acid sequences of SEQ ID NOs: 1576, 1577, and 1552, respectively; and the LCDR1, LCDR2, and LCDR3 comprise the amino acid sequences of SEQ ID NOs: 1585, 1565, and 1566, respectively;
(iii) the HCDR1, HCDR2, and HCDR3 comprise the amino acid sequences of SEQ ID NOs: 1594, 1595, and 1552, respectively; and the LCDR1, LCDR2, and LCDR3 comprise the amino acid sequences of SEQ ID NOs: 1601, 1602, and 1566, respectively;
(iv) the HCDR1, HCDR2, and HCDR3 comprise the amino acid sequences of SEQ ID NOs: 1503, 1504, and 1502, respectively; and LCDR1, LCDR2, and LCDR3 comprise the amino acid sequences of SEQ ID NOs: 1514, 1515, and 1516, respectively;
(v) the HCDR1, HCDR2, and HCDR3 comprise the amino acid sequences of SEQ ID NOs: 1528, 1529, and 1527, respectively; and the LCDR1, LCDR2, and LCDR3 comprise the amino acid sequences of SEQ ID NOs: 1539, 1540, and 1541, respectively;
(vi) the HCDR1, HCDR2, and HCDR3 comprise the amino acid sequences of SEQ ID NOs: 1611, 1612, and 1610, respectively; and the LCDR1, LCDR2, and LCDR3 comprise the amino acid sequences of SEQ ID NOs: 1621, 1622, and 1623, respectively;
(vii) the HC CDR1, HC CDR2, and HC CDR3 comprise the amino acid sequence of any of the HC CDR sequences of Tables 7-13; and the LC CDR1, LC CDR2, LC CDR3 comprise the amino acid sequence of any of the LC CDR sequences of Tables 7-13, or
(viii) the HCDR1, HCDR2, and/or HCDR3 sequences, and/or LCDR1, LCDR2, and/or LCDR3 sequences of any one of (i)-(vii), with at most one, two, or three substitutions (e.g., conservative amino acid substitutions) in each CDR.

31. The isolated AAV particle of any one of claims 1-30, wherein the antibody molecule comprises a heavy chain variable region (VH) comprising an HC CDR1, an HC CDR2, and an HC CDR3, and a light chain variable region (VL) comprising an LC CDR1, an LC CDR2, and an LC CDR3, wherein:
(i) the HCDR1, HCDR2, and HCDR3 comprise the amino acid sequences of SEQ ID NOs: 1631, 1551, and 1552, respectively; and the LCDR1, LCDR2, and LCDR3 comprise the amino acid sequences of SEQ ID NOs: 1632, 1633, and 1566, respectively;
(ii) the HCDR1, HCDR2, and HCDR3 comprise the amino acid sequences of SEQ ID NOs: 1634, 1635, and 1552, respectively; and the LCDR1, LCDR2, and LCDR3 comprise the amino acid sequences of SEQ ID NOs: 1636, 1633, and 1566, respectively; or
(iii) the HCDR1, HCDR2, and HCDR3 comprise the amino acid sequences of SEQ ID NOs: 1637, 1638, and 1557, respectively; and the LCDR1, LCDR2, and LCDR3 comprise the amino acid sequences of SEQ ID NOs: 1639, 1569, and 1566, respectively,
optionally wherein the antibody binds to a region of a human tau protein comprising residues 409-436, numbered according to SEQ ID NO: 9200.

32. The isolated AAV particle of any one of claims 1-31, wherein:
(i) the VH of the antibody molecule comprises the amino acid sequence of SEQ ID NO: 1562, 1512, 1537, 1583, 1599, or 1619 or an amino acid sequence having at least 90%, 92%, 95%, 96%, 97%, 98%, or 99% sequence identity thereto;
(ii) the VH of the antibody molecule comprises an amino acid sequence having at least one, two or three modifications, but not more than 30, 20 or 10 modifications of the amino acid sequence of SEQ ID NO: 1562, 1512, 1537, 1583, 1599, or 1619;
(iii) the VH of the antibody molecule comprises an amino acid sequence encoded by the nucleotide sequence of SEQ ID NO: 1563, 1513, 1538, 1584, 1600, or 1620, or a nucleotide sequence having at least 90%, 92%, 95%, 96%, 97%, 98%, or 99% sequence identity thereto; or
(iv) the VH of the antibody molecule comprises an amino acid sequence of any VH provided in Tables 7-13, or an amino acid sequence having at least 90%, 92%, 95%, 96%, 97%, 98%, or 99% sequence identity thereto; or an amino acid sequence having at least one, two or three modifications, but not more than 30, 20 or 10 modifications of the amino acid sequence of any VH provided in Tables 7-13.

33. The isolated AAV particle of any one of claims 1-32, wherein:
(i) the VL of the antibody molecule comprises the amino acid sequence of SEQ ID NO: 1573, 1523, 1548, 1591, 1606, or 1629, or an amino acid sequence having at least 90%, 92%, 95%, 96%, 97%, 98%, or 99% sequence identity thereto;
(ii) the VL of the antibody molecule comprises an amino acid sequence having at least one, two or three modifications, but not more than 30, 20 or 10 modifications of the amino acid sequence of SEQ ID NO: 1573, 1523, 1548, 1591, 1606, or 1629;
(iii) the VL of the antibody molecule comprises an amino acid sequence encoded by the nucleotide sequence of SEQ ID NO: 1574, 1524, 1549, 1592, 1607, or 1630, or a nucleotide sequence having at least 90%, 92%, 95%, 96%, 97%, 98%, or 99% sequence identity thereto; or
(iv) the VL of the antibody molecule comprises the amino acid sequence of any VL provided in Tables 7-13, or an amino acid sequence having at least 90%, 92%, 95%, 96%, 97%, 98%, or 99% sequence identity thereto; or an amino acid sequence having at least one, two or three modifications, but not more than 30, 20 or 10 modifications of the amino acid sequence of any VL provided in Tables 7-13.

34. The isolated AAV particle of any one of claims 1-33, wherein:
(i) the VH of the antibody molecule comprises the amino acid sequence of SEQ ID NO: 1562 or an amino acid sequence either having at least 80%, 85%, 90%, 92%, 95%, 96%, 97%, 98%, or 99% sequence identity thereto or having at least one, two or three modifications, but not more than 10 modifications thereof, and the VL of the antibody molecule comprises the amino acid sequence of SEQ ID NO: 1573 or an amino acid sequence either having at least 80%, 85%, 90%, 92%, 95%, 96%, 97%, 98%, or 99% sequence identity thereto or having at least one, two or three modifications, but not more than 10 modifications thereof;
(ii) the VH of the antibody molecule comprises the amino acid sequence of SEQ ID NO: 1512 or an amino acid sequence either having at least 80%, 85%, 90%, 92%, 95%, 96%, 97%, 98%, or 99% sequence identity thereto or having at least one, two or three modifications, but not more than 10 modifications thereof, and the VL of the antibody molecule comprises the amino acid sequence of SEQ ID NO: 1523 or an amino acid sequence either having at least 80%, 85%, 90%, 92%, 95%, 96%, 97%, 98%, or 99% sequence identity thereto or having at least one, two or three modifications, but not more than 10 modifications thereof;
(iii) the VH of the antibody molecule comprises the amino acid sequence of SEQ ID NO: 1537 or an amino acid sequence either having at least 80%, 85%, 90%, 92%, 95%, 96%, 97%, 98%, or 99% sequence identity thereto or having at least one, two or three modifications, but not more than 10 modifications thereof, and the VL of the antibody molecule comprises the amino acid sequence of SEQ ID NO: 1548 or an amino acid sequence either having at least 80%, 85%, 90%, 92%, 95%, 96%, 97%, 98%, or 99% sequence identity thereto or having at least one, two or three modifications, but not more than 10 modifications thereof;
(iv) the VH of the antibody molecule comprises the amino acid sequence of SEQ ID NO: 1583 or an amino acid sequence either having at least 80%, 85%, 90%, 92%, 95%, 96%, 97%, 98%, or 99% sequence identity thereto or having at least one, two or three modifications, but not more than 10 modifications thereof, and the VL of the antibody molecule comprises the amino acid sequence of SEQ ID NO: 1591 or an amino acid sequence either having at least 80%, 85%, 90%, 92%, 95%, 96%, 97%, 98%, or 99% sequence identity thereto or having at least one, two or three modifications, but not more than 10 modifications thereof;
(v) the VH of the antibody molecule comprises the amino acid sequence of SEQ ID NO: 1559 or an amino acid sequence either having at least 80%, 85%, 90%, 92%, 95%, 96%, 97%, 98%, or 99% sequence identity thereto or having at least one, two or three modifications, but not more than 10 modifications thereof, and the VL of the antibody molecule comprises the amino acid sequence of SEQ ID NO: 1606 or an amino acid sequence either having at least 80%, 85%, 90%, 92%, 95%, 96%, 97%, 98%, or 99% sequence identity thereto or having at least one, two or three modifications, but not more than 10 modifications thereof; or
(vi) the VH of the antibody molecule comprises the amino acid sequence of SEQ ID NO: 1619 or an amino acid sequence either having at least 80%, 85%, 90%, 92%, 95%, 96%, 97%, 98%, or 99% sequence identity thereto or having at least one, two or three modifications, but not more than 10 modifications thereof, and the VL of the antibody molecule comprises the amino acid sequence of SEQ ID NO: 1629 or an amino acid sequence either having at least 80%, 85%, 90%, 92%, 95%, 96%, 97%, 98%, or 99% sequence identity thereto or having at least one, two or three modifications, but not more than 10 modifications thereof.

35. The isolated AAV particle of any one of claims 1-34, wherein the antibody molecule comprises:
(i) a VH encoded by the nucleotide sequence of SEQ ID NO: 1563, or a nucleotide sequence having at least 80% (e.g., 85, 90, 95, 96, 97, 98, or 99%) sequence identity to SEQ ID NO: 1563; and a VL encoded by the nucleotide sequence of SEQ ID NO: 1574, or a nucleotide sequence having at least 80% (e.g., 85, 90, 95, 96, 97, 98, or 99%) sequence identity to SEQ ID NO: 1574;
(ii) a VH encoded by the nucleotide sequence of SEQ ID NO: 1513, or a nucleotide sequence having at least 80% (e.g., 85, 90, 95, 96, 97, 98, or 99%) sequence identity to SEQ ID NO: 1513; and a VL encoded by the nucleotide sequence of SEQ ID NO: 1524, or a nucleotide sequence having at least 80% (e.g., 85, 90, 95, 96, 97, 98, or 99%) sequence identity to SEQ ID NO: 1524;
(iii) a VH encoded by the nucleotide sequence of SEQ ID NO: 1538, or a nucleotide sequence having at least 80% (e.g., 85, 90, 95, 96, 97, 98, or 99%) sequence identity to SEQ ID NO: 1538; and a VL encoded by the nucleotide sequence of SEQ ID NO: 1549, or a nucleotide sequence having at least 80% (e.g., 85, 90, 95, 96, 97, 98, or 99%) sequence identity to SEQ ID NO: 1549;
(iv) a VH encoded by the nucleotide sequence of SEQ ID NO: 1584, or a nucleotide sequence having at least 80% (e.g., 85, 90, 95, 96, 97, 98, or 99%) sequence identity to SEQ ID NO: 1584; and a VL encoded by the nucleotide sequence of SEQ ID NO: 1592, or a nucleotide sequence having at least 80% (e.g., 85, 90, 95, 96, 97, 98, or 99%) sequence identity to SEQ ID NO: 1592;
(v) a VH encoded by the nucleotide sequence of SEQ ID NO: 1600, or a nucleotide sequence having at least 80% (e.g., 85, 90, 95, 96, 97, 98, or 99%) sequence identity to SEQ ID NO: 1600; and a VL encoded by the nucleotide sequence of SEQ ID NO: 1607, or a nucleotide sequence having at least 80% (e.g., 85, 90, 95, 96, 97, 98, or 99%) sequence identity to SEQ ID NO: 1607; or
(vi) a VH encoded by the nucleotide sequence of SEQ ID NO: 1620, or a nucleotide sequence having at least 80% (e.g., 85, 90, 95, 96, 97, 98, or 99%) sequence identity to SEQ ID NO: 1620; and a VL encoded by the nucleotide sequence of SEQ ID NO: 1630, or a nucleotide sequence having at least 80% (e.g., 85, 90, 95, 96, 97, 98, or 99%) sequence identity to SEQ ID NO: 1630.

36. The isolated AAV particle of any one of claims 1-29, wherein the antibody molecule comprises a heavy chain variable region (VH) comprising an HC CDR1, an HC CDR2, and an HC CDR3, and a light chain variable region (VL) comprising an LC CDR1, an LC CDR2, and an LC CDR3, wherein:
(i) the HCDR1, HCDR2, and HCDR3 comprise the amino acid sequences of SEQ ID NOs: 2200, 2201, and 2202, respectively; and the LCDR1, LCDR2, and LCDR3 comprise the amino acid sequences of SEQ ID NOs: 2207, 2208, and 2209, respectively;
(ii) the HCDR1, HCDR2, and HCDR3 comprise the amino acid sequences of SEQ ID NOs: 2232, 2233, and 2234, respectively; and the LCDR1, LCDR2, and LCDR3 comprise the amino acid sequences of SEQ ID NOs: 2238, 2239, and 2240, respectively;
(iii) the HCDR1, HCDR2, and HCDR3 comprise the amino acid sequences of SEQ ID NOs: 2248, 2219, and 2249, respectively; and the LCDR1, LCDR2, and LCDR3 comprise the amino acid sequences of SEQ ID NOs: 2207, 2208, and 2224, respectively;
(iv) the HCDR1, HCDR2, and HCDR3 comprise the amino acid sequences of SEQ ID NOs: 2218, 2219, and 2220, respectively; and the LCDR1, LCDR2, and LCDR3 comprise the amino acid sequences of SEQ ID NOs: 2207, 2208, and 2224, respectively;
(v) the HCDR1, HCDR2, and HCDR3 comprise the amino acid sequences of SEQ ID NOs: 2200, 2257, and 2258, respectively; and the LCDR1, LCDR2, and LCDR3 comprise the amino acid sequences of SEQ ID NOs: 2262, 2263, and 2264, respectively;
(vi) the HCDR1, HCDR2, and HCDR3 comprise the amino acid sequences of SEQ ID NOs: 2415, 2416, and 2417, respectively; and the LCDR1, LCDR2, and LCDR3 comprise the amino acid sequences of SEQ ID NOs: 2420, 2421, and 2422, respectively; or
(vii) the HCDR1, HCDR2, and/or HCDR3 sequences, and/or LCDR1, LCDR2, and/or LCDR3 sequences of any one of (i)-(vi), with at most one, two, or three substitutions (e.g., conservative amino acid substitutions) in each CDR.

37. The isolated AAV particle of any one of claims 1-29 and 36, wherein:
(i) the VH of the antibody molecule comprises the amino acid sequence of SEQ ID NO: 2214, 2244, 2253, 2228, 2269, or 2426, or an amino acid sequence having at least 90%, 92%, 95%, 96%, 97%, 98%, or 99% sequence identity thereto;
(ii) the VH of the antibody molecule comprises an amino acid sequence having at least one, two or three modifications, but not more than 30, 20 or 10 modifications of the amino acid sequence of SEQ ID NO: 2214, 2244, 2253, 2228, 2269, or 2426;
(iii) the VH of the antibody molecule comprises an amino acid sequence encoded by the nucleotide sequence of SEQ ID NO: 2216, 2246, 2255, 2230, 2271, or 2428, or a nucleotide sequence having at least 90%, 92%, 95%, 96%, 97%, 98%, or 99% sequence identity thereto; or
(iv) the VH of the antibody molecule comprises an amino acid sequence of any VH provided in Tables 14-16, or an amino acid sequence having at least 90%, 92%, 95%, 96%, 97%, 98%, or 99% sequence identity thereto; or an amino acid sequence having at least one, two or three modifications, but not more than 30, 20 or 10 modifications of the amino acid sequence of any VH provided in Tables 14-16.

38. The isolated AAV particle of any one of claims 1-29, 36, and 37, wherein:
(i) the VL of the antibody molecule comprises the amino acid sequence of SEQ ID NO: 2215, 2245, 2254, 2229, 2270, or 2427, or an amino acid sequence having at least 90%, 92%, 95%, 96%, 97%, 98%, or 99% sequence identity thereto;
(ii) the VL of the antibody molecule comprises an amino acid sequence having at least one, two or three modifications, but not more than 30, 20 or 10 modifications of the amino acid sequence of SEQ ID NO: 2215, 2245, 2254, 2229, 2270, or 2427;
(iii) the VL of the antibody molecule comprises an amino acid sequence encoded by the nucleotide sequence of SEQ ID NO: 2217, 2247, 2256, 2231, 2272, or 2429, or a nucleotide sequence having at least 90%, 92%, 95%, 96%, 97%, 98%, or 99% sequence identity thereto; or
(iv) the VL of the antibody molecule comprises the amino acid sequence of any VL provided in Tables 14-16, or an amino acid sequence having at least 90%, 92%, 95%, 96%, 97%, 98%, or 99% sequence identity thereto; or an amino acid sequence having at least one, two or three modifications, but not more than 30, 20 or 10 modifications of the amino acid sequence of any VL provided in Tables 14-16.

39. The isolated AAV particle of any one of claims 1-29 or 36-38, wherein:
(i) the VH of the antibody molecule comprises the amino acid sequence of SEQ ID NO: 2214 or an amino acid sequence either having at least 80%, 85%, 90%, 92%, 95%, 96%, 97%, 98%, or 99% sequence identity thereto or having at least one, two or three modifications, but not more than 10 modifications thereof, and the VL of the antibody molecule comprises the amino acid sequence of SEQ ID NO: 2215 or an amino acid sequence either having at least 80%, 85%, 90%, 92%, 95%, 96%, 97%, 98%, or 99% sequence identity thereto or having at least one, two or three modifications, but not more than 10 modifications thereof;
(ii) the VH of the antibody molecule comprises the amino acid sequence of SEQ ID NO: 2244 or an amino acid sequence either having at least 80%, 85%, 90%, 92%, 95%, 96%, 97%, 98%, or 99% sequence identity thereto or having at least one, two or three modifications, but not more than 10 modifications thereof, and the VL of the antibody molecule comprises the amino acid sequence of SEQ ID NO: 2245 or an amino acid sequence either having at least 80%, 85%, 90%, 92%, 95%, 96%, 97%, 98%, or 99% sequence identity thereto or having at least one, two or three modifications, but not more than 10 modifications thereof;
(iii) the VH of the antibody molecule comprises the amino acid sequence of SEQ ID NO: 2253 or an amino acid sequence either having at least 80%, 85%, 90%, 92%, 95%, 96%, 97%, 98%, or 99% sequence identity thereto or having at least one, two or three modifications, but not more than 10 modifications thereof, and the VL of the antibody molecule comprises the amino acid sequence of SEQ ID NO: 2254 or an amino acid sequence either having at least 80%, 85%, 90%, 92%, 95%, 96%, 97%, 98%, or 99% sequence identity thereto or having at least one, two or three modifications, but not more than 10 modifications thereof;
(iv) the VH of the antibody molecule comprises the amino acid sequence of SEQ ID NO: 2228 or an amino acid sequence either having at least 80%, 85%, 90%, 92%, 95%, 96%, 97%, 98%, or 99% sequence identity thereto or having at least one, two or three modifications, but not more than 10 modifications thereof, and the VL of the antibody molecule comprises the amino acid sequence of SEQ ID NO: 2229 or an amino acid sequence either having at least 80%, 85%, 90%, 92%, 95%, 96%, 97%, 98%, or 99% sequence identity thereto or having at least one, two or three modifications, but not more than 10 modifications thereof;
(v) the VH of the antibody molecule comprises the amino acid sequence of SEQ ID NO: 2269 or an amino acid sequence either having at least 80%, 85%, 90%, 92%, 95%, 96%, 97%, 98%, or 99% sequence identity thereto or having at least one, two or three modifications, but not more than 10 modifications thereof, and the VL of the antibody molecule comprises the amino acid sequence of SEQ ID NO: 2270 or an amino acid sequence either having at least 80%, 85%, 90%, 92%, 95%, 96%, 97%, 98%, or 99% sequence identity thereto or having at least one, two or three modifications, but not more than 10 modifications thereof; or
(vi) the VH of the antibody molecule comprises the amino acid sequence of SEQ ID NO: 2426 or an amino acid sequence either having at least 80%, 85%, 90%, 92%, 95%, 96%, 97%, 98%, or 99% sequence identity thereto or having at least one, two or three modifications, but not more than 10 modifications thereof, and the VL of the antibody molecule comprises the amino acid sequence of SEQ ID NO: 2427 or an amino acid sequence either having at least 80%, 85%, 90%, 92%, 95%, 96%, 97%, 98%, or 99% sequence identity thereto or having at least one, two or three modifications, but not more than 10 modifications thereof.

40. The isolated AAV particle of any one of claims 1-29 or 36-39, wherein the antibody molecule comprises:
(i) a VH encoded by the nucleotide sequence of SEQ ID NO: 2216, or a nucleotide sequence having at least 80% (e.g., 85, 90, 95, 96, 97, 98, or 99%) sequence identity to SEQ ID NO: 2216; and a VL encoded by the nucleotide sequence of SEQ ID NO: 2217, or a nucleotide sequence having at least 80% (e.g., 85, 90, 95, 96, 97, 98, or 99%) sequence identity to SEQ ID NO: 2217;
(ii) a VH encoded by the nucleotide sequence of SEQ ID NO: 2246, or a nucleotide sequence having at least 80% (e.g., 85, 90, 95, 96, 97, 98, or 99%) sequence identity to SEQ ID NO: 2246; and a VL encoded by the nucleotide sequence of SEQ ID NO: 2247, or a nucleotide sequence having at least 80% (e.g., 85, 90, 95, 96, 97, 98, or 99%) sequence identity to SEQ ID NO: 2247;
(iii) a VH encoded by the nucleotide sequence of SEQ ID NO: 2255, or a nucleotide sequence having at least 80% (e.g., 85, 90, 95, 96, 97, 98, or 99%) sequence identity to SEQ ID NO: 2255; and a VL encoded by the nucleotide sequence of SEQ ID NO: 2256, or a nucleotide sequence having at least 80% (e.g., 85, 90, 95, 96, 97, 98, or 99%) sequence identity to SEQ ID NO: 2256;
(iv) a VH encoded by the nucleotide sequence of SEQ ID NO: 2230, or a nucleotide sequence having at least 80% (e.g., 85, 90, 95, 96, 97, 98, or 99%) sequence identity to SEQ ID NO: 2230; and a VL encoded by the nucleotide sequence of SEQ ID NO: 2231, or a nucleotide sequence having at least 80% (e.g., 85, 90, 95, 96, 97, 98, or 99%) sequence identity to SEQ ID NO: 2231;
(v) a VH encoded by the nucleotide sequence of SEQ ID NO: 2271, or a nucleotide sequence having at least 80% (e.g., 85, 90, 95, 96, 97, 98, or 99%) sequence identity to SEQ ID NO: 2271; and a VL encoded by the nucleotide sequence of SEQ ID NO: 2272, or a nucleotide sequence having at least 80% (e.g., 85, 90, 95, 96, 97, 98, or 99%) sequence identity to SEQ ID NO: 2272; or
(vi) a VH encoded by the nucleotide sequence of SEQ ID NO: 2428, or a nucleotide sequence having at least 80% (e.g., 85, 90, 95, 96, 97, 98, or 99%) sequence identity to SEQ ID NO: 2428; and a VL encoded by the nucleotide sequence of SEQ ID NO: 2429, or a nucleotide sequence having at least 80% (e.g., 85, 90, 95, 96, 97, 98, or 99%) sequence identity to SEQ ID NO: 2429.

41. The isolated AAV particle of any one of claims 1-40, wherein the antibody molecule is a full length antibody, a bispecific antibody, an Fab, an F(ab′)₂, an Fv, a single chain Fv fragment (scFv), a diabody, a single chain antibody, an intrabody, a nanobody, or a camelid antibody.

42. The isolated AAV particle of any one of claims 1-41, wherein the antibody molecule comprises a heavy chain constant region selected from human IgG1, human IgG2, human IgG3, human IgG4, murine IgG1, murine IgG2a, murine IgG2b, murine IgG2c, and murine IgG3; and/or a light chain constant region selected from the light chain constant regions of kappa or lambda.

43. The isolated AAV particle of any one of claims 1-42, wherein the antibody molecule comprises:
(i) a heavy chain constant region comprising the amino acid sequence of any one of SEQ ID NOs: 2474-2476 or 2478-2481, or a sequence having at least 80% (e.g., 85, 90, 95, 96, 97, 98, or 99%) sequence identity to any one of SEQ ID NOs: 2474-2476 or 2478-2481, an amino acid sequence having at least one, two or three modifications, but not more than 30, 20 or 10 modifications, to the amino acid sequence of any one of SEQ ID NOs: 2474-2476 or 2478-2481, or an amino acid sequence of a heavy chain constant region provided in Table 17; and/or
(ii) a light chain constant region (CL) comprising the amino acid sequence of any one of SEQ ID NOs: 2482, 2484-2487, or 2489-2497, or a sequence having at least 80% (e.g., 85, 90, 95, 96, 97, 98, or 99%) sequence identity to any one of SEQ ID NOs: 2482, 2484-2487, or 2489-2497, or an amino acid sequence having at least one, two or three modifications, but not more than 30, 20 or 10 modifications, to the amino acid sequence of any one of SEQ ID NOs: 2482, 2484-2487, or 2489-2497, or an amino acid sequence of a light chain constant region provided in Table 17.

44. The isolated AAV particle of any one of claims 1-43, further comprising a viral genome comprising a promoter operably linked to the nucleic acid comprising the transgene encoding the antibody molecule, wherein the promoter comprises a tissue specific promoter or a ubiquitous promoter.

45. The isolated AAV particle of claim 44, wherein the promoter comprises:
(i) a chicken j-actin (CBA) promoter and/or its derivative CAG, an EF-1α promoter, a CMV immediate-early enhancer and/or promoter, a 3 glucuronidase (GUSB) promoter, a ubiquitin C (UBC) promoter, a neuron-specific enolase (NSE), a platelet-derived growth factor (PDGF) promoter, a platelet-derived growth factor B-chain (PDGF-β) promoter, an intercellular adhesion molecule 2 (ICAM-2) promoter, a synapsin (Syn) promoter, a methyl-CpG binding protein 2 (MeCP2) promoter, a Ca2+/calmodulin-dependent protein kinase II (CaMKII) promoter, a metabotropic glutamate receptor 2 (mGluR2) promoter, a neurofilament light (NFL) or heavy (NFH) promoter, a β-globin minigene nβ2 promoter, a preproenkephalin (PPE) promoter, an enkephalin (Enk) and excitatory amino acid transporter 2 (EAAT2), a glial fibrillary acidic protein (GFAP) promoter, a myelin basic protein (MBP) promoter, a cardiovascular promoter (e.g., αMHC, cTnT, and CMV-MLC2k), a liver promoter (e.g., hAAT, TBG), a skeletal muscle promoter (e.g., desmin, MCK, C512) or a fragment, e.g., a truncation, or a functional variant thereof; or
(ii) the nucleotide sequence of any one of SEQ ID NOs: 9000, 9001, 9003, 9004, 9008, 9009, 9011-9020, GCATG, CGTGAG, GT, GCTCCGGT, or GTAAG, or a nucleotide sequence at least 80% (e.g., 85%, 90%, 95%, 96%, 97%, 98%, or 99%) identical thereto or any of the sequences provided in Table 6.

46. The isolated AAV particle of claim 44 or 45, wherein the viral genome further comprises an enhancer, optionally wherein the enhancer is a CMV immediate-early (CMVie) enhancer.

47. The isolated AAV particle of any one of claims 44-46, wherein the viral genome further comprises:
(i) a polyadenylation (polyA) signal region, optionally wherein the polyA signal region comprises the nucleotide sequence of any of SEQ ID NO: 2038, or a nucleotide sequence with at least 95% identity thereto;
(ii) an ITR sequence, optionally wherein:
(a) the ITR sequence is positioned 5′ relative to the encoded payload and/or the ITR sequence is positioned 3′ relative to the encoded; and/or
(b) the ITR sequence comprises a nucleotide sequence of SEQ ID NO: 2026 or 2029, or a nucleotide sequence with at least 80%, 85%, 90%, or 95% sequence identity thereto;
(iii) at least 1, 2, or 3 intron regions, optionally wherein the intron region comprises the nucleotide sequence of SEQ ID NO: 2029, or a nucleotide sequence with at least 95% identity thereto;
(iv) at least 1, 2, or 3 exon regions; optionally wherein each exon region independently encodes a VH, VL, heavy chain constant region, light chain constant region, heavy chain, or light chain of the antibody molecule, e.g., an antibody molecule or constant region provided in any one of Tables 7-17, or a nucleotide sequence with at least 95% identity thereto; and/or
(v) a Kozak sequence, optionally wherein the Kozak sequence comprises the nucleotide sequence of GCCGCCACCATG (SEQ ID NO: 9021) or GAGGAGCCACC (SEQ ID NO: 9022).

48. The isolated AAV particle of any one of claims 44-47, wherein the viral genome further comprises a nucleotide sequence encoding at least 1-5 copies of an miR binding site, e.g., at least 1, 2, 3, 4, or 5 copies, optionally wherein the at least 1-5 copies of the encoded miR binding sites are continuous, e.g., not separated by a spacer; or are separated by a spacer (optionally wherein the spacer comprises the nucleotide sequence of GATAGTTA).

49. The isolated AAV particle of claim 48, wherein the encoded miR binding site comprises a miR183 binding site, a miR122 binding site, a miR-1 binding site, a miR-142-3p, or a combination thereof, optionally wherein:
(i) the encoded miR183 binding site comprises the nucleotide sequence of SEQ ID NO: 3675, or a nucleotide sequence having at least 85%, 90%, 92%, 95%, 97%, 98%, or 99% sequence identity thereto; or a nucleotide sequence having at least one, two, three, four, five, six, or seven modifications, but no more than ten modifications of SEQ ID NO: 3675;
(ii) the encoded miR122 binding site comprises the nucleotide sequence of SEQ ID NO: 3672, or a nucleotide sequence having at least 85%, 90%, 92%, 95%, 97%, 98%, or 99% sequence identity thereto; or a nucleotide sequence having at least one, two, three, four, five, six, or seven modifications, but no more than ten modifications of SEQ ID NO: 3672;
(iii) the encoded miR-1 binding site comprises the nucleotide sequence of SEQ ID NO: 4679, or a nucleotide sequence having at least 85%, 90%, 92%, 95%, 97%, 98%, or 99% sequence identity thereto; or a nucleotide sequence having at least one, two, three, four, five, six, or seven modifications, but no more than ten modifications of SEQ ID NO: 4679; and/or
(iv) the encoded miR-142-3p binding site comprises the nucleotide sequence of SEQ ID NO: 3674, or a nucleotide sequence having at least 70%, 75%, 80%, 85%, 90%, 92%, 95%, 97%, 98%, or 99% sequence identity thereto; or a nucleotide sequence having at least one, two, three, four, five, six, or seven modifications, but no more than ten modifications of SEQ ID NO: 3674.

50. The isolated AAV particle of any one of claims 44-49, wherein the viral genome comprises in 5′ to 3′ order:
(i) a 5′ adeno-associated (AAV) ITR, optionally wherein the 5′ AAV ITR comprises the nucleotide sequence of SEQ ID NO: 2026, or a nucleotide sequence at least 95% identical thereto;
(ii) a CMVie enhancer, optionally wherein the CMVie enhancer comprises the nucleotide sequence of SEQ ID NO: 2027, or a nucleotide sequence at least 95% identical thereto;
(iii) a CBA promoter or functional variant thereof, optionally wherein the CBA promoter or functional variant thereof comprises the nucleotide sequence of SEQ ID NO: 2028, or a nucleotide sequence at least 95% identical thereto;
(iv) an intron, optionally wherein the intron comprises the nucleotide sequence of SEQ ID NO: 2029, or a nucleotide sequence at least 95% identical thereto;
(v) a nucleotide sequence encoding a signal sequence, optionally wherein the nucleotide sequence encoding the signal sequence comprises the nucleotide sequence of SEQ ID NO: 2030, or a nucleotide sequence at least 95% identical thereto;
(vi) a transgene encoding a heavy chain variable region (VH) of the antibody molecule, wherein the nucleotide sequence encoding the VH comprises the nucleotide sequence of SEQ ID NO: 2031 or a nucleotide sequence at least 80% (e.g., at least 85, 90, 95, 96, 97, 98, or 99%) identical thereto;
(vii) a nucleotide sequence encoding an IgG1 heavy chain constant region, optionally wherein the nucleotide sequence encoding the IgG1 heavy chain constant region comprises the nucleotide sequence of SEQ ID NO: 2032, or a nucleotide sequence at least 80% (e.g., at least 85, 90, 95, 96, 97, 98, or 99%) identical thereto;
(viii) a furin cleavage site, optionally wherein the furin cleavage site comprises the nucleotide sequence of SEQ ID NO: 2033, or a nucleotide sequence at least 95% identical thereto;
(ix) a T2A linker, optionally wherein the T2A linker comprises the nucleotide sequence of SEQ ID NO: 2034, or a nucleotide sequence at least 95% identical thereto;
(x) a nucleotide sequence encoding a signal sequence, optionally wherein the nucleotide sequence encoding the signal sequence comprises the nucleotide sequence of SEQ ID NO: 2035, or a nucleotide sequence at least 95% identical thereto;
(xi) a transgene encoding a light chain variable region (VL) of the antibody molecule, wherein the nucleotide sequence encoding the VL comprises the nucleotide sequence of SEQ ID NO: 2036 or a nucleotide sequence at least 80% (e.g., at least 85, 90, 95, 96, 97, 98, or 99%) identical thereto;
(xii) a nucleotide sequence encoding an IgG kappa light chain constant region, optionally wherein the nucleotide sequence encoding the IgG kappa light chain constant region comprises the nucleotide sequence of SEQ ID NO: 2037, or a nucleotide sequence at least 80% (e.g., at least 85, 90, 95, 96, 97, 98, or 99%) identical thereto;
(xiii) a polyA signal region, optionally wherein the polyA signal region comprises the nucleotide sequence of SEQ ID NO: 2038, or a nucleotide sequence at least 95% identical thereto; and
(xiv) a 3′ AAV ITR, optionally wherein the 3′ AAV ITR comprises the nucleotide sequence of SEQ ID NO: 2039, or a nucleotide sequence at least 95% identical thereto.

51. The isolated AAV particle of any one of claims 44-50, wherein the viral genome comprises the nucleotide sequence of SEQ ID NO: 2025, or a nucleotide sequence at least 80% (e.g., at least 85, 90, 95, 96, 97, 98, or 99%) identical thereto.

52. The isolated AAV particle of any one of claims 44-51, wherein the viral genome:
(i) is single stranded;
(ii) further comprises a nucleic acid encoding a capsid protein, e.g., a structural protein, wherein the capsid protein comprises a VP1 polypeptide, a VP2 polypeptide, and/or a VP3 polypeptide, optionally wherein the VP1 polypeptide, the VP2 polypeptide, and/or the VP3 polypeptide are encoded by at least one Cap gene; and/or
(iii) further comprises a nucleic acid encoding a Rep protein, e.g., a non-structural protein, wherein the Rep protein comprises a Rep78 protein, a Rep68, Rep52 protein, and/or a Rep40 protein. optionally wherein the Rep78 protein, the Rep68 protein, the Rep52 protein, and/or the Rep40 protein are encoded by at least one Rep gene.

53. A cell comprising the isolated AAV particle of any one of claims 1-52, optionally wherein the cell is a mammalian cell, e.g., an HEK293 cell, an insect cell, e.g., an Sf9 cell, or a bacterial cell.

54. A method of making the isolated AAV particle of any one of claims 1-52, the method comprising
(i) providing a host cell comprising the viral genome; and
(ii) incubating the host cell under conditions suitable to enclose the viral genome in the AAV capsid variant;
thereby making the isolated AAV particle.

55. A pharmaceutical composition comprising the isolated AAV particle of any one of claims 1-52, and a pharmaceutically acceptable excipient.

56. A method of delivering an antibody molecule which binds to tau (e.g., human tau) to a subject, comprising administering an effective amount of the pharmaceutical composition of claim 55 or the isolated AAV particle of any one of claims 1-52, thereby delivering the antibody molecule to the subject.

57. The method of claim 56, wherein the subject has, has been diagnosed with having, or is at risk of having:
(i) a disease associated with tau expression or activity;
(ii) a neurological disorder (e.g., a neurodegenerative disorder);
(iii) a tau-related disease (e.g., a tauopathy).

58. A method of treating a subject having or diagnosed with having a disease associated with tau expression or activity comprising administering an effective amount of the pharmaceutical composition of claim 55 or the isolated AAV particle of any one of claims 1-52, thereby treating the disease associated with tau expression in the subject.

59. A method of treating a subject having or diagnosed with having a neurological disorder (e.g., a neurodegenerative disorder), comprising administering an effective amount of the pharmaceutical composition of claim 55 or the isolated AAV particle of any one of claims 1-52, thereby treating the neurodegenerative disorder in the subject.

60. A method of treating a subject having or diagnosed with having a tauopathy, comprising administering an effective amount of the pharmaceutical composition of claim 55 or the isolated AAV particle of any one of claims 1-52, thereby treating the tauopathy in the subject.

61. The method of any one of claims 57-60, wherein the disease associated with tau expression or activity, the neurological disorder (e.g., neurodegenerative disorder), or tauopathy comprises Alzheimer's disease (AD), frontotemporal dementia and parkinsonism linked to chromosome 17 (FTDP-17), frontotemporal lobar degeneration (FTLD), frontotemporal dementia (FTD), chronic traumatic encephalopathy (CTE), progressive Supranuclear Palsy (PSP), Down's syndrome, Pick's disease, corticobasal degeneration (CBD), corticobasal syndrome, amyotrophic lateral sclerosis (ALS), prion diseases, Creutzfeldt-Jakob disease (CJD), multiple system atrophy, tangle-only dementia, or progressive subcortical gliosis.

62. The method of any one of claims 56-61, wherein the AAV particle is administered to the subject:
(i) intravenously, intracerebrally, via intrathalamic (ITH) administration, intramuscularly, intrathecally, intracerebroventricularly, via intraparenchymal administration, via focused ultrasound (FUS), e.g., coupled with the intravenous administration of microbubbles (FUS-MB), or MRI-guided FUS coupled with intravenous administration, via intra-cisterna magna injection (ICM), or via dual ITH and ICM administration; or
(ii) via intravenous administration, optionally wherein the intravenous administration is via focused ultrasound (FUS), e.g., coupled with the intravenous administration of microbubbles (FUS-MB), or MRI-guided FUS coupled with intravenous administration.

63. The method of any one of claims 56-62, further comprising administration of an additional therapeutic agent and/or therapy suitable for treatment or prevention of the disease associated with tau expression or activity, a neurological disorder (e.g., a neurodegenerative disorder), or a tauopathy, optionally wherein the additional therapeutic agent comprises enzyme replacement therapy (ERT) (e.g., imiglucerase, velaglucerase alfa, or taliglucerase alfa); substrate reduction therapy (SRT) (e.g., eliglustat or miglustat), blood transfusion, levodopa, carbidopa, Safinamide, dopamine agonists (e.g., pramipexole, rotigotine, or ropinirole), anticholinergics (e.g., benztropine or trihexyphenidyl), cholinesterase inhibitors (e.g., rivastigmine, donepezil, or galantamine), an N-methyl-d-aspartate (NMDA) receptor antagonist (e.g., memantine), or a combination thereof.

64. The method of any one of claims 56-63, wherein the AAV particle is administered to the subject at a dose of:
(a) about 6.7e11 VG/kg to 2e13 VG/kg (e.g., 6.7e11 VG/kg, 2e12 VG/kg, 6.7e12 VG/kg, or 2e13 VG/kg) or about 5e11 VG/kg to 3e13 VG/kg;
(b) about 6.7e10 VG/kg to 6.7e12 VG/kg, about 1.3e11 VG/kg to 3.4e12 VG/kg, or about 2.2e11 VG/kg to 2e12 VG/kg;
(c) about 4e11 VG/kg to 8e11 VG/kg (e.g., about 6.7e11 VG/kg);
(d) about 2e11 VG/kg to 2e13 VG/kg, about 4e11 VG/kg to 1e13 VG/kg, about 6.7e11 VG/kg to about 6e12 VG/kg;
(e) about 1e12 VG/kg to 5e12 VG/kg (e.g., about 2e12 VG/kg);
(f) about 6.7e11 VG/kg to 6.7e13 VG/kg, about 1.3e12 VG/kg to 3.4e13 VG/kg, or about 2.2e12 VG/kg to 2e13 VG/kg;
(g) about 4e12 VG/kg to 8e12 VG/kg (e.g., about 6.7e12 VG/kg);
(h) about 2e12 VG/kg to 2e14 VG/kg, about 4e12 VG/kg to 1e14 VG/kg, about 6.7e12 VG/kg to about 6e13 VG/kg; or
(i) about 1e13 VG/kg to 5e13 VG/kg (e.g., about 2e13 VG/kg).

65. The pharmaceutical composition of claim 55 or the isolated AAV particle of any one of claims 1-52, for use in the manufacture of a medicament.

66. The pharmaceutical composition of claim 55 or the isolated AAV particle of any one of claims 1-52, for use in the treatment of a disease associated with tau expression or activity, a neurological disorder (e.g., a neurodegenerative disorder), or a tauopathy.

67. Use of the pharmaceutical composition of claim 55 or the isolated AAV particle of any one of claims 1-52, in the manufacture of a medicament for the treatment of a disease associated with tau expression or activity, a neurological disorder (e.g., a neurodegenerative disorder), or a tauopathy.
